(19) United States
(12) Patent Application Publication
(10) Pub. No.: US 2004/0086875 A1
(43) Pub. Date: May 6, 2004

(54) Novel proteins and nucleic acids encoding same

(76) Inventors: Michele L. Agee, Wallingford, CT (US); John P. Alsobrook II, Madison, CT (US); Constance Berghs, New Haven, CT (US); Ferenc L. Boldog, North Haven, CT (US); Catherine E. Burgess, Wethersfield, CT (US); John S. Chant, Branford, CT (US); Amitabha Chaudhuri, Madison, CT (US); Vincent A. DiPippo, East Haven, CT (US); Shlomit R. Edinger, New Haven, CT (US); Andrew Eisen, Rockville, MD (US); Karen Ellerman, Branford, CT (US); Esha A. Gangolli, Madison, CT (US); Linda Gorman, Branford, CT (US); Valerie Gerlach, Branford, CT (US); Weizhen Ji, Branford, CT (US); Ramesh Kekuda, Norwalk, CT (US); Nikolai V. Khramtsov, Branford, CT (US); Li Li, Branford, CT (US); Uriel M. Malyankar, Branford, CT (US); John R. MacDougall, Hamden, CT (US); Peter S. Mezes, Old Lyme, CT (US); Charles E. Miller, Guilford, CT (US); Isabelle Millet, Milford, CT (US); Chean Eng. Ooi, Branford, CT (US); Tatiana Ort, Milford, CT (US); Muralidhara Padigaru, Branford, CT (US); Meera Patturajan, Branford, CT (US); Luca Rastelli, Guilford, CT (US); Daniel K. Rieger, Branford, CT (US); Mark E. Rothenberg, Clinton, CT (US); Suresh G. Shenoy, Branford, CT (US); Steven K. Spaderna, Berlin, CT (US); Kimberly A. Spytek, New Haven, CT (US); Raymond J. Taupier JR., East Haven, CT (US); Corine A.M. Vernet, Branford, CT (US); Bryan D. Zerhusen, Branford, CT (US); Mei Zhong, Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/287,226

(22) Filed: Nov. 4, 2002

Related U.S. Application Data
(60) Provisional application No. 60/334,421, filed on Nov. 30, 2001.
(60) Provisional application No. 60/354,392, filed on Feb. 4, 2002.
(60) Provisional application No. 60/360,148, filed on Feb. 27, 2002.
(60) Provisional application No. 60/364,000, filed on Mar. 13, 2002.
(60) Provisional application No. 60/404,821, filed on Aug. 20, 2002.
(60) Provisional application No. 60/334,526, filed on Nov. 30, 2001.
(60) Provisional application No. 60/354,409, filed on Feb. 4, 2002.
(60) Provisional application No. 60/364,227, filed on Mar. 13, 2002.
(60) Provisional application No. 60/334,027, filed on Nov. 28, 2001.
(60) Provisional application No. 60/331,641, filed on Nov. 20, 2001.
(60) Provisional application No. 60/335,610, filed on Nov. 15, 2001.
(60) Provisional application No. 60/333,461, filed on Nov. 27, 2001.
(60) Provisional application No. 60/403,619, filed on Aug. 15, 2002.
(60) Provisional application No. 60/336,664, filed on Dec. 4, 2001.
(60) Provisional application No. 60/361,925, filed on Mar. 5, 2002.
(60) Provisional application No. 60/405,631, filed on Aug. 23, 2002.
(60) Provisional application No. 60/333,072, filed on Nov. 6, 2001.
(60) Provisional application No. 60/338,314, filed on Dec. 7, 2001.
(60) Provisional application No. 60/354,393, filed on Feb. 4, 2002.
(60) Provisional application No. 60/361,790, filed on Mar. 5, 2002.
(60) Provisional application No. 60/364,182, filed on Mar. 13, 2002.
(60) Provisional application No. 60/353,288, filed on Feb. 1, 2002.
(60) Provisional application No. 60/362,230, filed on Mar. 5, 2002.
(60) Provisional application No. 60/364,181, filed on Mar. 13, 2002.
(60) Provisional application No. 60/338,390, filed on Dec. 7, 2001.
(60) Provisional application No. 60/361,833, filed on Mar. 5, 2002.
(60) Provisional application No. 60/405,368, filed on Aug. 23, 2002.
(60) Provisional application No. 60/339,008, filed on Dec. 10, 2001.
(60) Provisional application No. 60/362,625, filed on Mar. 5, 2002.
(60) Provisional application No. 60/364,197, filed on Mar. 13, 2002.
(60) Provisional application No. 60/401,594, filed on Aug. 7, 2002.
(60) Provisional application No. 60/405,402, filed on Aug. 23, 2002.
(60) Provisional application No. 60/339,006, filed on Dec. 10, 2001.
(60) Provisional application No. 60/353,280, filed on Feb. 1, 2002.
(60) Provisional application No. 60/359,944, filed on Feb. 27, 2002.
(60) Provisional application No. 60/405,496, filed on Aug. 23, 2002.
(60) Provisional application No. 60/333,072, filed on Nov. 6, 2001.
(60) Provisional application No. 60/338,626, filed on Nov. 5, 2001.
(60) Provisional application No. 60/348,283, filed on Nov. 9, 2001.
(60) Provisional application No. 60/335,610, filed on Nov. 15, 2001.
(60) Provisional application No. 60/331,641, filed on Nov. 20, 2001.
(60) Provisional application No. 60/331,630, filed on Nov. 20, 2001.
(60) Provisional application No. 60/332,152, filed on Nov. 21, 2001.
(60) Provisional application No. 60/334,300, filed on Nov. 29, 2001.
(60) Provisional application No. 60/401,787, filed on Aug. 7, 2002.
(60) Provisional application No. 60/396,703, filed on Jul. 17, 2002.
(60) Provisional application No. 60/401,552, filed on Aug. 6, 2002.
(60) Provisional application No. 60/336,576, filed on Dec. 4, 2001.
(60) Provisional application No. 60/335,610, filed on Nov. 15, 2001.
(60) Provisional application No. 60/381,621, filed on May 17, 2002.
(60) Provisional application No. 60/383,675, filed on May 28, 2002.
(60) Provisional application No. 60/406,125, filed on Aug. 26, 2002.
(60) Provisional application No. 60/338,543, filed on Nov. 16, 2001.
(60) Provisional application No. 60/339,286, filed on Dec. 11, 2001.
(60) Provisional application No. 60/336,576, filed on Dec. 4, 2001.
(60) Provisional application No. 60/333,912, filed on Nov. 28, 2001.

(51) Int. Cl. C12Q 1/68; C07H 21/04; C12N 9/00; C12P 21/02; C12N 5/06; C07K 14/47; A61K 38/17

(52) U.S. Cl. 435/6; 435/69.1; 435/183; 435/320.1; 435/325; 514/12; 530/350; 536/23.2

(57) ABSTRACT

The present invention provides novel isolated polynucleotides and small molecule target polypeptides encoded by the polynucleotides. Antibodies that immunospecifically bind to a novel small molecule target polypeptide or any derivative, variant, mutant or fragment of that polypeptide, polynucleotide or antibody are disclosed, as are methods in which the small molecule target polypeptide, polynucleotide and antibody are utilized in the detection and treatment of a broad range of pathological states. More specifically, the present invention discloses methods of using recombinantly expressed and/or endogenously expressed proteins in various screening procedures for the purpose of identifying therapeutic antibodies and therapeutic small molecules associated with diseases. The invention further discloses therapeutic, diagnostic and research methods for diagnosis, treatment, and prevention of disorders involving any one of these novel human nucleic acids and proteins.

### RELATED APPLICATIONS

This application claims priority to provisional patent applications U.S. S. No. 60/334421, filed Nov. 30, 2001; U.S. S. No. 60/354392, filed Feb. 4, 2002; U.S. S. No. 60/360148, filed Feb. 27, 2002; U.S. S. No. 60/364000, filed Mar. 13, 2002; U.S. S. No. 60/404821, filed Aug. 20, 2002; U.S. S. No. 60/334526, filed Nov. 30, 2001; U.S. S. No. 60/354409, filed Feb. 4, 2002; U.S. S. No. 60/364227, filed Mar. 13, 2002; U.S. S. No. 60/334027, filed Nov. 28, 2001; U.S. S. No. 60/331641, filed Nov. 20, 2001; U.S. S. No. 60/335610, filed Nov. 15, 2001; U.S. S. No. 60/333461, filed Nov. 27, 2001; U.S. S. No. 60/403619, filed Aug. 15, 2002; U.S. S. No. 60/336664, filed Dec. 4, 2001; U.S. S. No. 60/361925, filed Mar. 5, 2002; U.S. S. No. 60/405631, filed Aug. 23, 2002; U.S. S. No. 60/333072, filed Nov. 6, 2001; U.S. S. No. 60/338314, filed Dec. 7, 2001; U.S. S. No. 60/354393, filed Feb. 4, 2002; U.S. S. No. 60/361790, filed Mar. 5, 2002; U.S. S. No. 60/364182, filed Mar. 13, 2002; U.S. S. No. 60/353288, filed Feb. 1, 2002; U.S. S. No. 60/362230, filed Mar. 5, 2002; U.S. S. No. 60/364181, filed Mar. 13, 2002; U.S. S. No. 60/338390, filed Dec. 7, 2001; U.S. S. No. 60/361833, filed Mar. 5, 2002; U.S. S. No. 60/405,368, filed Aug. 23, 2002; U.S. S. No. 60/339008, filed Dec. 10, 2001; U.S. S. No. 60/362625, filed Mar. 5, 2002; U.S. S. No. 60/364197, filed Mar. 13, 2002; U.S. S. No. 60/401594, filed Aug. 7, 2002; U.S. S. No. 60/405402, filed Aug. 23, 2002; U.S. S. No. 60/339006, filed Dec. 10, 2001; U.S. S. No. 60/353280, filed Feb. 1, 2002; U.S. S. No. 60/359944, filed Feb. 27, 2002; U.S. S. No. 60/405,496, filed Aug. 23, 2002; U.S. S. No. 60/333072, filed Nov. 6, 2001; U.S. S. No. 60/338626, filed Nov. 5, 2001; U.S. S. No. 60/348283, filed Nov. 9, 2001; U.S. S. No. 60/335610, filed Nov. 15,2001; U.S. S. No. 60/331641, filed Nov. 20,2001; U.S. S. No. 60/331630, filed Nov. 20, 2001; U.S. S. No. 60/332152, filed Nov. 21, 2001; U.S. S. No. 60/401787, filed Aug. 7, 2002; U.S. S. No. 60/396703, filed Jul. 17, 2002; U.S. S. No. 60/401552, filed Aug. 6, 2002; U.S. S. No. 60/336576, filed Dec. 4, 2001; U.S. S. No. 60/335610, filed Nov. 15, 2001; U.S. S. No. 60/381621, filed May 17, 2002; U.S. S. No. 60/383675, filed May 28, 2002; U.S. S. No. 60/406125, filed Aug. 26, 2002; U.S. S. No. 60/338543, filed Nov. 16, 2001; U.S. S. No. 60/339286, filed Dec. 11, 2001; U.S. S. No. 60/336576, filed Dec. 4, 2001; U.S. S. No. 60/333912, filed Nov. 28, 2001; each of which is incorporated herein by reference in its entirety.

### FIELD OF THE INVENTION

The present invention relates to novel polypeptides that are targets of small molecule drugs and that have properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Small molecule targets have been implicated in various disease states or pathologies. These targets may be proteins, and particularly enzymatic proteins, which are acted upon by small molecule drugs for the purpose of altering target function and achieving a desired result. Cellular, animal and clinical studies can be performed to elucidate the genetic contribution to the etiology and pathogenesis of conditions in which small molecule targets are implicated in a variety of physiologic, pharmacologic or native states. These studies utilize the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify potential avenues for therapeutic intervention in order to prevent, treat the consequences or cure the conditions.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

In many cases the objective of such screening assays is to identify small molecule candidates; this is commonly approached by the use of combinatorial methodologies to develop the population of substances to be tested. The implementation of high throughput screening methodologies is advantageous when working with large, combinatorial libraries of compounds.

### SUMMARY OF THE INVENTION

The invention includes nucleic acid sequences and the novel polypeptides they encode. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid, which represents the nucleotide sequence selected from the group consisting of SEQ ID NO: 2n−1, wherein n is an integer between 1 and 226, or polypeptide sequences, which represents the group consisting of SEQ ID NO: 2n, wherein n is an integer between 1 and 226.

In one aspect, the invention provides an isolated polypeptide comprising a mature form of a NOVX amino acid. One example is a variant of a mature form of a NOVX amino acid sequence, wherein any amino acid in the mature form is changed to a different amino acid, provided that no more than 15% of the amino acid residues in the sequence of the mature form are so changed. The amino acid can be, for example, a NOVX amino acid sequence or a variant of a NOVX amino acid sequence, wherein any amino acid specified in the chosen sequence is changed to a different amino acid, provided that no more than 15% of the amino acid residues in the sequence are so changed. The invention also includes fragments of any of these. In another aspect, the invention also includes an isolated nucleic acid that encodes a NOVX polypeptide, or a fragment, homolog, analog or derivative thereof.

Also included in the invention is a NOVX polypeptide that is a naturally occurring allelic variant of a NOVX sequence. In one embodiment, the allelic variant includes an amino acid sequence that is the translation of a nucleic acid sequence differing by a single nucleotide from a NOVX nucleic acid sequence. In another embodiment, the NOVX polypeptide is a variant polypeptide described therein, wherein any amino acid specified in the chosen sequence is changed to provide a conservative substitution. In one embodiment, the invention discloses a method for determining the presence or amount of the NOVX polypeptide in a sample. The method involves the steps of: providing a sample; introducing the sample to an antibody that binds immunospecifically to the polypeptide; and determining the presence or amount of antibody bound to the NOVX polypeptide, thereby determining the presence or amount of the NOVX polypeptide in the sample. In another embodiment, the invention provides a method for determining the presence of or predisposition to a disease associated with altered levels of a NOVX polypeptide in a mammalian subject. This method involves the steps of: measuring the level of expression of the polypeptide in a sample from the first mammalian subject; and comparing the amount of the polypeptide in the sample of the first step to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In a further embodiment, the invention includes a method of identifying an agent that binds to a NOVX polypeptide. This method involves the steps of: introducing the polypeptide to the agent; and determining whether the agent binds to the polypeptide. In various embodiments, the agent is a cellular receptor or a downstream effector.

In another aspect, the invention provides a method for identifying a potential therapeutic agent for use in treatment of a pathology, wherein the pathology is related to aberrant expression or aberrant physiological interactions of a NOVX polypeptide. The method involves the steps of: providing a cell expressing the NOVX polypeptide and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent. In another aspect, the invention describes a method for screening for a modulator of activity or of latency or predisposition to a pathology associated with the NOVX polypeptide. This method involves the following steps: administering a test compound to a test animal at increased risk for a pathology associated with the NOVX polypeptide, wherein the test animal recombinantly expresses the NOVX polypeptide. This method involves the steps of measuring the activity of the NOVX polypeptide in the test animal after administering the compound of step; and comparing the activity of the protein in the test animal with the activity of the NOVX polypeptide in a control animal not administered the polypeptide, wherein a change in the activity of the NOVX polypeptide in the test animal relative to the control animal indicates the test compound is a modulator of latency of, or predisposition to, a pathology associated with the NOVX polypeptide. In one embodiment, the test animal is a recombinant test animal that expresses a test protein transgene or expresses the transgene under the control of a promoter at an increased level relative to a wild-type test animal, and wherein the promoter is not the native gene promoter of the transgene. In another aspect, the invention includes a method for modulating the activity of the NOVX polypeptide, the method comprising introducing a cell sample expressing the NOVX polypeptide with a compound that binds to the polypeptide in an amount sufficient to modulate the activity of the polypeptide.

The invention also includes an isolated nucleic acid that encodes a NOVX polypeptide, or a fragment, homolog, analog or derivative thereof. In a preferred embodiment, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In another embodiment, the nucleic acid molecule differs by a single nucleotide from a NOVX nucleic acid sequence. In one embodiment, the NOVX nucleic acid molecule hybridizes under stringent conditions to the nucleotide sequence selected from the group consisting of SEQ ID NO: 2n−1, wherein n is an integer between 1 and 226, or a complement of the nucleotide sequence. In another aspect, the invention provides a vector or a cell expressing a NOVX nucleotide sequence.

In one embodiment, the invention discloses a method for modulating the activity of a NOVX polypeptide. The method includes the steps of: introducing a cell sample expressing the NOVX polypeptide with a compound that binds to the polypeptide in an amount sufficient to modulate the activity of the polypeptide. In another embodiment, the invention includes an isolated NOVX nucleic acid molecule comprising a nucleic acid sequence encoding a polypeptide comprising a NOVX amino acid sequence or a variant of a mature form of the NOVX amino acid sequence, wherein any amino acid in the mature form of the chosen sequence is changed to a different amino acid, provided that no more than 15% of the amino acid residues in the sequence of the mature form are so changed. In another embodiment, the invention includes an amino acid sequence that is a variant of the NOVX amino acid sequence, in which any amino acid specified in the chosen sequence is changed to a different amino acid, provided that no more than 15% of the amino acid residues in the sequence are so changed.

In one embodiment, the invention discloses a NOVX nucleic acid fragment encoding at least a portion of a NOVX polypeptide or any variant of the polypeptide, wherein any amino acid of the chosen sequence is changed to a different amino acid, provided that no more than 10% of the amino acid residues in the sequence are so changed.

In another embodiment, the invention includes the complement of any of the NOVX nucleic acid molecules or a naturally occurring allelic nucleic acid variant. In another embodiment, the invention discloses a NOVX nucleic acid molecule that encodes a variant polypeptide, wherein the variant polypeptide has the polypeptide sequence of a naturally occurring polypeptide variant. In another embodiment, the invention discloses a NOVX nucleic acid, wherein the nucleic acid molecule differs by a single nucleotide from a NOVX nucleic acid sequence.

In another aspect, the invention includes a NOVX nucleic acid, wherein one or more nucleotides in the NOVX nucleotide sequence is changed to a different nucleotide provided that no more than 15% of the nucleotides are so changed. In one embodiment, the invention discloses a nucleic acid fragment of the NOVX nucleotide sequence and a nucleic acid fragment wherein one or more nucleotides in the NOVX nucleotide sequence is changed from that selected from the group consisting of the chosen sequence to a different nucleotide provided that no more than 15% of the nucleotides are so changed. In another embodiment, the invention includes a nucleic acid molecule wherein the nucleic acid molecule hybridizes under stringent conditions to a NOVX nucleotide sequence or a complement of the NOVX nucleotide sequence. In one embodiment, the invention includes a nucleic acid molecule, wherein the sequence is changed such that no more than 15% of the nucleotides in the coding sequence differ from the NOVX nucleotide sequence or a fragment thereof.

In a further aspect, the invention includes a method for determining the presence or amount of the NOVX nucleic acid in a sample. The method involves the steps of: providing the sample; introducing the sample to a probe that binds to the nucleic acid molecule; and determining the presence or amount of the probe bound to the NOVX nucleic acid molecule, thereby determining the presence or amount of the NOVX nucleic acid molecule in the sample. In one embodiment, the presence or amount of the nucleic acid molecule is used as a marker for cell or tissue type.

In another aspect, the invention discloses a method for determining the presence of or predisposition to a disease associated with altered levels of the NOVX nucleic acid molecule of in a first mammalian subject. The method involves the steps of: measuring the amount of NOVX nucleic acid in a sample from the first mammalian subject; and comparing the amount of the nucleic acid in the sample of step (a) to the amount of NOVX nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| NOVX | Internal | SEQ ID NO | SEQ ID NO | |
|---|---|---|---|---|
| Assignment | Identification | (nucleic acid) | (amino acid) | Homology |
| 1a | CG101683-01 | 1 | 2 | Mitogen-activated protein |
| | | | | kinase kinase kinase 8 |
| 1b | 248490507 | 3 | 4 | Mitogen-activated protein |
| | | | | kinase kinase kinase 8 |
| 1c | 253174293 | 5 | 6 | Mitogen-activated protein |
| | | | | kinase kinase kinase 8 |
| 1d | 248490584 | 7 | 8 | Mitogen-activated protein |
| | | | | kinase kinase kinase 8 |
| 1e | 258054391 | 9 | 10 | Mitogen-activated protein |
| | | | | kinase kinase kinase 8 |
| 1f | 248494549 | 11 | 12 | Mitogen-activated protein |
| | | | | kinase kinase kinase 8 |
| 1g | 259741837 | 13 | 14 | Mitogen-activated protein |
| | | | | kinase kinase kinase 8 |
| 1h | 260480803 | 15 | 16 | Mitogen-activated protein |
| | | | | kinase kinase kinase 8 |
| 1i | 209983329 | 17 | 18 | Mitogen-activated protein |
| | | | | kinase kinase kinase 8 |
| 1j | 212779055 | 19 | 20 | Mitogen-activated protein |
| | | | | kinase kinase kinase 8 |
| 1k | 212779063 | 21 | 22 | Mitogen-activated protein |
| | | | | kinase kinase kinase 8 |
| 1l | CG101683-02 | 23 | 24 | Mitogen-activated protein |
| | | | | kinase kinase kinase 8 |
| 1m | CG101683-03 | 25 | 26 | Mitogen-activated protein |
| | | | | kinase kinase kinase 8 |
| 1n | CG101683-04 | 27 | 28 | Mitogen-activated protein |
| | | | | kinase kinase kinase 8 |
| 1o | CG101683-05 | 29 | 30 | Mitogen-activated protein |
| | | | | kinase kinase kinase 8 |
| 1p | CG101683-06 | 31 | 32 | Mitogen-activated protein |
| | | | | kinase kinase kinase 8 |
| 1q | CG101683-07 | 33 | 34 | Mitogen-activated protein |
| | | | | kinase kinase kinase 8 |
| 1r | CG101683-08 | 35 | 36 | Mitogen-activated protein |
| | | | | kinase kinase kinase 8 |
| 2a | CG101996-01 | 37 | 38 | Phosphorylase B kinase |
| | | | | gamma catalytic chain, |
| | | | | skeletal muscle isoform |
| 2b | CG101996-04 | 39 | 40 | Phosphorylase B kinase |
| | | | | gamma catalytic chain, |
| | | | | skeletal muscle isoform |
| 2c | CG101996-02 | 41 | 42 | Phosphorylase B kinase |
| | | | | gamma catalytic chain, |
| | | | | skeletal muscle isoform |
| 2d | 245245680 | 43 | 44 | Phosphorylase B kinase |
| | | | | gamma catalytic chain, |
| | | | | skeletal muscle isoform |
| 2e | 245245707 | 45 | 46 | Phosphorylase B kinase |
| | | | | gamma catalytic chain, |
| | | | | skeletal muscle isoform |
| 2f | 248494552 | 47 | 48 | Phosphorylase B kinase |
| | | | | gamma catalytic chain, |
| | | | | skeletal muscle isoform |
| 2g | 242435676 | 49 | 50 | Phosphorylase B kinase |
| | | | | gamma catalytic chain, |
| | | | | skeletal muscle isoform |
| 2h | 254868664 | 51 | 52 | Phosphorylase B kinase |
| | | | | gamma catalytic chain, |
| | | | | skeletal muscle isoform |
| 2i | 249122191 | 53 | 54 | Phosphorylase B kinase |
| | | | | gamma catalytic chain, |
| | | | | skeletal muscle isoform |
| 2j | 249122234 | 55 | 56 | Phosphorylase B kinase |
| | | | | gamma catalytic chain, |
| | | | | skeletal muscle isoform |
| 2k | CG101996-03 | 57 | 58 | Phosphorylase B kinase |
| | | | | gamma catalytic chain, |
| | | | | skeletal muscle isoform |
| 2l | CG101996-05 | 59 | 60 | Phosphorylase B kinase |
| | | | | gamma catalytic chain, |
| | | | | skeletal muscle isoform |
| 2m | CG101996-06 | 61 | 62 | Phosphorylase B kinase |
| | | | | gamma catalytic chain, |
| | | | | skeletal muscle isoform |
| 2n | CG101996-07 | 63 | 64 | Phosphorylase B kinase |
| | | | | gamma catalytic chain, |
| | | | | skeletal muscle isoform |
| 2o | CG101996-08 | 65 | 66 | Phosphorylase B kinase |
| | | | | gamma catalytic chain, |
| | | | | skeletal muscle isoform |
| 2p | CG101996-09 | 67 | 68 | Phosphorylase B kinase |
| | | | | gamma catalytic chain, |
| | | | | skeletal muscle isoform |
| 3a | CG102822-01 | 69 | 70 | glutamate-ammonia |
| | | | | ligase |
| 3b | CG102822-03 | 71 | 72 | glutamate-ammonia |
| | | | | ligase |
| 3c | CG102822-03 | 73 | 74 | glutamate-5ammonia |
| | | | | ligase |
| 3d | CG102822-04 | 75 | 76 | glutamate-ammonia |
| | | | | ligase |
| 4a | CG103241-01 | 77 | 78 | Beta-1,4- |
| | | | | galactosyltransferase 2 |
| 4b | CG103241-02 | 79 | 80 | Beta-1,4- |
| | | | | galactosyltransferase 2 |
| 4c | CG103241-03 | 81 | 82 | Beta-1,4- |
| | | | | galactosyltransferase 2 |
| 5a | CG106249-01 | 83 | 84 | KIAA 1590 protein |
| 5b | CG106249-02 | 85 | 86 | KIAA 1590 protein |
| 6a | CG106824-01 | 87 | 88 | Tryptase beta-1 precursor |
| 6b | CG106824-04 | 89 | 90 | Tryptase beta-1 precursor |
| 6c | CG106824-02 | 91 | 92 | Tryptase beta-1 precursor |
| 6d | CG106824-03 | 93 | 94 | Tryptase beta-1 precursor |
| 7a | CG114327-01 | 95 | 96 | Similar to hypothetical |
| | | | | protein FLJ23469 |
| 7b | CG114327-02 | 97 | 98 | Similar to hypothetical |
| | | | | protein FLJ23469 |
| 8a | CG119418-01 | 99 | 100 | Farnesyl-diphosphate |
| | | | | farnesyltransferase |
| 9a | CG120359-01 | 101 | 102 | Acetyl-coenzyme A |
| | | | | synthetase, cytoplasmic |
| 9b | 277685717 | 103 | 104 | Acetyl-coenzyme A |
| | | | | synthetase, cytoplasmic |
| 9c | 277686882 | 105 | 106 | Acetyl-coenzyme A |
| | | | | synthetase, cytoplasmic |
| 9d | CG120359-02 | 107 | 108 | Acetyl-coenzyme A |
| | | | | synthetase, cytoplasmic |
| 10a | CG124907-01 | 109 | 110 | Ornithine decarboxylase |
| 10b | CG124907-01 | 111 | 112 | Ornithine decarboxylase |
| 10c | 254048022 | 113 | 114 | Ornithine decarboxylase |
| 10d | 258252457 | 115 | 116 | Ornithine decarboxylase |
| 10e | 258280014 | 117 | 118 | Ornithine decarboxylase |
| 10f | 258330318 | 119 | 120 | Ornithine decarboxylase |
| 10g | 258330346 | 121 | 122 | Ornithine decarboxylase |
| 10h | 258330472 | 123 | 124 | Ornithine decarboxylase |
| 10i | 258330611 | 125 | 126 | Ornithine decarboxylase |
| 10j | 260481330 | 127 | 128 | Ornithine decarboxylase |
| 10k | CG124907-02 | 129 | 130 | Ornithine decarboxylase |
| 10l | CG124907-03 | 131 | 132 | Ornithine decarboxylase |
| 10m | CG124907-04 | 133 | 134 | Ornithine decarboxylase |
| 10n | CG124907-05 | 135 | 136 | Ornithine decarboxylase |
| 10o | CG124907-06 | 137 | 138 | Ornithine decarboxylase |
| 11a | CG128347-01 | 139 | 140 | Hypothetical 96.7 kDa |
| | | | | protein |
| 11b | CG128347-02 | 141 | 142 | Hypothetical 96.7 kDa |
| | | | | protein |
| 12a | CG135823-01 | 143 | 144 | Tyrosine |
| | | | | aminotransferase |
| 12b | CG135823-02 | 145 | 146 | Tyrosine |
| | | | | aminotransferase |
| 12c | 233048273 | 147 | 148 | Tyrosine |
| | | | | aminotransferase |
| 12d | 233048286 | 149 | 150 | Tyrosine |
| | | | | aminotransferase |
| 12e | 248490358 | 151 | 152 | Tyrosine |
| | | | | aminotransferase |
| 12f | 254868693 | 153 | 154 | Tyrosine |
| | | | | aminotransferase |
| 12g | 255667122 | 155 | 156 | Tyrosine |
| | | | | aminotransferase |
| 12h | 258252417 | 157 | 158 | Tyrosine |
| | | | | aminotransferase |
| 12i | 259741773 | 159 | 160 | Tyrosine |
| | | | | aminotransferase |
| 12j | 260480043 | 161 | 162 | Tyrosine |
| | | | | aminotransferase |
| 12k | CG135823-03 | 163 | 164 | Tyrosine |
| | | | | aminotransferase |
| 12l | CG135823-04 | 165 | 166 | Tyrosine |
| | | | | aminotransferase |
| 13a | CG140122-01 | 167 | 168 | Polyamine oxidase |
| | | | | isoform-1 - Homo sapiens |
| 13b | 246864043 | 169 | 170 | Polyamine oxidase |
| | | | | isoform-1 - Homo sapiens |
| 13c | 246864086 | 171 | 172 | Polyamine oxidase |
| | | | | isoform-1 - Homo sapiens |
| 13d | 258280083 | 173 | 174 | Polyamine oxidase |
| | | | | isoform-1 - Homo sapiens |
| 13e | 258280066 | 175 | 176 | Polyamine oxidase |
| | | | | isoform-1 - Homo sapiens |
| 13f | 258329988 | 177 | 178 | Polyamine oxidase |
| | | | | isoform-1 - Homo sapiens |
| 13g | 254047897 | 179 | 180 | Polyamine oxidase |
| | | | | isoform-1 - Homo sapiens |
| 13h | 258329988 | 181 | 182 | Polyamine oxidase |
| | | | | isoform-1 - Homo sapiens |
| 13i | 258280066 | 183 | 184 | Polyamine oxidase |
| | | | | isoform-1 - Homosapiens |
| 13j | 258280083 | 185 | 186 | Polyamine oxidase |
| | | | | isoform-1 - Homo sapiens |
| 13k | CG140122-02 | 187 | 188 | Polyamine oxidase |
| | | | | isoform-1 - Homo sapiens |
| 13l | CG140122-03 | 189 | 190 | Polyamine oxidase |
| | | | | isoform-1 - Homo sapiens |
| 13m | CG140122-04 | 191 | 192 | Polyamine oxidase |
| | | | | isoform-1 - Homo sapiens |
| 13n | CG140122-05 | 193 | 194 | Polyamine oxidase |
| | | | | isoform-1 - Homo sapiens |
| 13o | CG140122-06 | 195 | 196 | Polyamine oxidase |
| | | | | isoform-1 - Homo sapiens |
| 13p | CG140122-07 | 197 | 198 | Polyamine oxidase |
| | | | | isoform-1 - Homo sapiens |
| 13q | CG140122-08 | 199 | 200 | Polyamine oxidase |
| | | | | isoform-1 - Homo sapiens |
| 14a | CG140316-01 | 201 | 202 | NADP-dependent malic |
| | | | | enzyme |
| 14b | CG140316-01 | 203 | 204 | NADP-dependent malic |
| | | | | enzyme |
| 14c | 254047949 | 205 | 206 | NADP-dependent malic |
| | | | | enzyme |
| 14d | 258280122 | 207 | 208 | NADP-dependent malic |
| | | | | enzyme |
| 14e | 258330149 | 209 | 210 | NADP-dependent malic |
| | | | | enzyme |
| 14f | 258330422 | 211 | 212 | NADP-dependent malic |
| | | | | enzyme |
| 14g | 258330562 | 213 | 214 | NADP-dependent malic |
| | | | | enzyme |
| 14h | 258330639 | 215 | 216 | NADP-dependent malic |
| | | | | enzyme |
| 14i | 259357792 | 217 | 218 | NADP-dependent malic |
| | | | | enzyme |
| 14j | CG140316-02 | 219 | 220 | NADP-dependent malic |
| | | | | enzyme |
| 14k | CG140316-03 | 221 | 222 | NADP-dependent malic |
| | | | | enzyme |
| 14l | CG140316-04 | 223 | 224 | NADP-dependent malic |
| | | | | enzyme |
| 15a | CG142427-01 | 225 | 226 | ATP-citrate (pro-S—)-lyase |
| 15b | CG142427-01 | 227 | 228 | ATP-citrate (pro-S—)-lyase |
| 15c | CG142427-04 | 229 | 230 | ATP-citrate (pro-S—)-lyase |
| 15d | CG142427-02 | 231 | 232 | ATP-citrate (pro-S—)-lyase |
| 15e | CG142427-03 | 233 | 234 | ATP-citrate (pro-S—)-lyase |
| 15f | 256388552 | 235 | 236 | ATP-citrate (pro-S—)-lyase |
| 15g | 256420210 | 237 | 238 | ATP-citrate (pro-S—)-lyase |
| 15h | 256202925 | 239 | 240 | ATP-citrate (pro-S—)-lyase |
| 15i | 259856081 | 241 | 242 | ATP-citrate (pro-S—)-lyase |
| 15j | 256388552 | 243 | 244 | ATP-citrate (pro-S—)-lyase |
| 15k | 256420210 | 245 | 246 | ATP-citrate (pro-S—)-lyase |
| 15l | 256202925 | 247 | 248 | ATP-citrate (pro-S—)-lyase |
| 15m | 296463359 | 249 | 250 | ATP-citrate (pro-S—)-lyase |
| 15n | 263470992 | 251 | 252 | ATP-citrate (pro-S—)-lyase |
| 15o | CG142427-05 | 253 | 254 | ATP-citrate (pro-S—)-lyase |
| 16a | CG142631-01 | 255 | 256 | L-serine dehydratase |
| 16b | CG142631-01 | 257 | 258 | L-serine dehydratase |
| 16c | 248494617 | 259 | 260 | L-serine dehydratase |
| 16d | 228832711 | 261 | 262 | L-serine dehydratase |
| 16e | 256420310 | 263 | 264 | L-serine dehydratase |
| 16f | 249117058 | 265 | 266 | L-serine dehydratase |
| 16g | 252790334 | 267 | 268 | L-serine dehydratase |
| 16h | 254869149 | 269 | 270 | L-serine dehydratase |
| 16i | CG142631-02 | 271 | 272 | L-serine dehydratase |
| 16j | CG142631-03 | 273 | 274 | L-serine dehydratase |
| 16k | CG142631-04 | 275 | 276 | L-serine dehydratase |
| 17a | CG151359-01 | 277 | 278 | L-lactate dehydrogenase |
| | | | | A-like |
| 18a | CG152227-01 | 279 | 280 | Similar to 3- |
| | | | | hydroxyisobutyryl- |
| | | | | coenzyme A hydrolase |
| 18b | CG152227-02 | 281 | 282 | Similar to 3- |
| | | | | hydroxyisobutyryl- |
| | | | | coenzyme A hydrolase |
| 19a | CG152392-01 | 283 | 284 | Hypothetical 68.5 kDa |
| | | | | protein |
| 20a | CG152453-01 | 285 | 286 | Beta-1,4- |
| | | | | galactosyltransferase 6 |
| 20b | CG152453-03 | 287 | 288 | Beta-1,4- |
| | | | | galactosyltransferase 6 |
| 20c | CG152453-02 | 289 | 290 | Beta-1,4- |
| | | | | galactosyltransferase 6 |
| 21a | CG152547-01 | 291 | 292 | Hypothetical 26.3 kDa |
| | | | | protein |
| 22a | CG152646-01 | 293 | 294 | Hypothetical 57.5 kDa |
| | | | | protein |
| 23a | CG152959-01 | 295 | 296 | CAAX prenyl protease 2 |
| 23b | CG152959-02 | 297 | 298 | CAAX prenyl protease 2 |
| 24a | CG153033-01 | 299 | 300 | Vesicular glutamate |
| | | | | transporter 3 - Homo |
| | | | | sapiens |
| 25a | CG153818-01 | 301 | 302 | CDNA FLJ37300 fis, |
| | | | | clone BRAMY2015782, |
| | | | | moderately similar to |
| | | | | KINESIN-LIKE |
| | | | | PROTEIN |
| 26a | CG154435-01 | 303 | 304 | Dynein beta chain, ciliary |
| 27a | CG154465-01 | 305 | 306 | Similar to hypothetical |
| | | | | protein DKFZp434G2226 - |
| 28a | CG154492-01 | 307 | 308 | High-affinity cGMP- |
| | | | | specific 3′,5′-cyclic |
| | | | | phosphodiesterase 9A |
| 28b | CG154492-02 | 309 | 310 | High-affinity cGMP- |
| | | | | specific 3′,5′-cyclic |
| | | | | phosphodiesterase 9A |
| 29a | CG154509-01 | 311 | 312 | Cytoplasmic dynein |
| | | | | heavy chain |
| 30a | CG155595-01 | 313 | 314 | Hypothetical 98.5 kDa |
| | | | | protein |
| 31a | CG155962-01 | 315 | 316 | Kinesin-like protein |
| | | | | KIF1B (Klp) |
| 32a | CG157477-01 | 317 | 318 | Myosin I |
| 33a | CG157486-01 | 319 | 320 | EphA2 |
| 34a | CG157505-01 | 321 | 322 | KIAA 1300 protein |
| 35a | CG157629-01 | 323 | 324 | Serine/threonine protein |
| | | | | phosphatase with EF- |
| | | | | hands-1 |
| 35b | CG157629-01 | 325 | 326 | Serine/threonine protein |
| | | | | phosphatase with EF- |
| | | | | hands-1 |
| 36a | CG157704-01 | 327 | 328 | Probable mitotic |
| | | | | centromere associated |
| | | | | kinesin - Leishmania |
| | | | | major |
| 37a | CG158218-01 | 329 | 330 | Kinesin-related protein |
| | | | | 3A |
| 38a | CG158513-01 | 331 | 332 | Prostatic acid phosphatase |
| | | | | precursor |
| 38b | CG158513-02 | 333 | 334 | Prostatic acid phosphatase |
| | | | | precursor |
| 39a | CG158583-01 | 335 | 336 | Synaptic vesicle amine |
| | | | | transporter (Monoamine |
| | | | | transporter) (Vesicular |
| | | | | amine transporter 2) |
| | | | | (VAT2) |
| 39b | CG158583-02 | 337 | 338 | Synaptic vesicle amine |
| | | | | transporter (Monoamine |
| | | | | transporter) (Vesicular |
| | | | | amine transporter 2) |
| | | | | (VAT2) |
| 39c | CG158583-04 | 339 | 340 | Synaptic vesicle amine |
| | | | | transporter (Monoamine |
| | | | | transporter) (Vesicular |
| | | | | amine transporter 2) |
| | | | | (VAT2) |
| 39d | CG158583-05 | 341 | 342 | Synaptic vesicle amine |
| | | | | transporter (Monoamine |
| | | | | transporter) (Vesicular |
| | | | | amine transporter 2) |
| | | | | (VAT2) |
| 39e | CG158583-03 | 343 | 345 | Synaptic vesicle amine |
| | | | | transporter (Monoamine |
| | | | | transporter) (Vesicular |
| | | | | amine transporter 2) |
| | | | | (VAT2) |
| 40a | CG158964-01 | 346 | 347 | PHOSPHATIDIC acid |
| | | | | phosphatase 2A |
| 40b | CG158964-02 | 348 | 349 | PHOSPHATIDIC acid |
| | | | | phosphatase 2A |
| 41a | CG159084-01 | 349 | 350 | Glutamate decarboxylase |
| | | | | 67 |
| 42a | CG159130-01 | 351 | 352 | Hyperpolarization- |
| | | | | activated cation channel, |
| | | | | HAC2 |
| 43a | CG159178-01 | 353 | 354 | Carbonic anhydrase VI |
| | | | | precursor (EC 4.2.1.1) |
| | | | | (Carbonate dehydratase |
| | | | | VI) (CA-VI) (Secreted |
| | | | | carbonic anhydrase) |
| | | | | (Salivary carbonic |
| | | | | anhydrase) |
| 43b | CG159178-02 | 355 | 356 | Carbonic anhydrase VI |
| | | | | precursor (EC 4.2.1.1) |
| | | | | (Carbonate dehydratase |
| | | | | VI) (CA-VI) (Secreted |
| | | | | carbonic anhydrase) |
| | | | | (Salivary carbonic |
| | | | | anhydrase) |
| 44a | CG160131-01 | 357 | 358 | Glycerol kinase (EC |
| | | | | 2.7.1.30) (ATP: glycerol |
| | | | | 3-phosphotransferase) |
| | | | | (Glycerokinase) (GK) |
| 44b | CG160131-04 | 359 | 360 | Glycerol kinase (EC |
| | | | | 2.7.1.30) (ATP: glycerol |
| | | | | 3-phosphotransferase) |
| | | | | (Glycerokinase) (GK) |
| 44c | CG160131-02 | 361 | 362 | Glycerol kinase (EC |
| | | | | 2.7.1.0) (ATP: glycerol |
| | | | | 3-phosphotransferase) |
| | | | | (Glycerokinase) (GK) |
| 44d | CG160131-03 | 363 | 364 | Glycerol kinase (EC |
| | | | | 2.7.1.30) (ATP: glycerol |
| | | | | 3-phosphotransferase) |
| | | | | (Glycerokinase) (GK) |
| 45a | CG166282-01 | 365 | 366 | Serine/threonine-protein |
| | | | | kinase Chk1 (EC 2.7.1.-) |
| 46a | CG170739-01 | 367 | 368 | Pendrin (Sodium- |
| | | | | independent |
| | | | | chloride/iodide |
| | | | | transporter) |
| 47a | CG171632-01 | 369 | 370 | Gamma-aminobutyric- |
| | | | | acid receptor rho-1 |
| | | | | subunit precursor |
| | | | | (GABA(A) receptor) |
| 47b | CG171632-01 | 371 | 372 | Gamma-aminobutyric- |
| | | | | acid receptor rho-1 |
| | | | | subunit precursor |
| | | | | (GABA(A) receptor) |
| 48a | CG173066-01 | 373 | 374 | Aquaporin 7 (Aquaporin- |
| | | | | 7 like) (Aquaporin |
| | | | | adipose) (AQPap) |
| 49a | CG173085-01 | 375 | 376 | Similar to thyroid |
| | | | | hormone receptor |
| 49b | 311531811 | 377 | 378 | Similar to thyroid |
| | | | | hormone receptor |
| 50a | CG173095-01 | 379 | 380 | Ubiquitin-protein ligase |
| | | | | E3 Mdm2 (EC 6.3.2.-) |
| | | | | (p53-binding protein |
| | | | | Mdm2) (Oncoprotein |
| | | | | Mdm2) (Double minute 2 |
| | | | | protein) (Hdm2) |
| 50b | CG173095-02 | 381 | 382 | Ubiquitin-protein ligase |
| | | | | E3 Mdm2 (EC 6.3.2.-) |
| | | | | (p53-binding protein |
| | | | | Mdm2) (Oncoprotein |
| | | | | Mdm2) (Double minute 2 |
| | | | | protein) (Hdm2) |
| 51a | CG173173-01 | 383 | 384 | Gamma-aminobutyric- |
| | | | | acid receptor alpha-5 |
| | | | | subunit precursor |
| | | | | (GABA(A) receptor) |
| 52a | CG51213-01 | 385 | 386 | Sequence 3 from Patent |
| | | | | WO0123561 |
| 52b | CG51213-07 | 387 | 388 | Sequence 3 from Patent |
| | | | | WO0123561 |
| 52c | CG51213-02 | 389 | 390 | Sequence 3 from Patent |
| | | | | WO0123561 |
| 52d | CG51213-03 | 391 | 392 | Sequence 3 from Patent |
| | | | | WO0123561 |
| 52e | CG51213-04 | 393 | 394 | Sequence 3 from Patent |
| | | | | WO0123561 |
| 52f | CG51213-05 | 395 | 396 | Sequence 3 from Patent |
| | | | | WO0123561 |
| 52g | CG51213-06 | 397 | 398 | Sequence 3 from Patent |
| | | | | WO0123561 |
| 53a | CG56155-01 | 399 | 400 | Plasma kallikrein |
| | | | | precursor (EC 3.4.21.34) |
| | | | | (Plasma prekallikrein) |
| | | | | (Kininogenin) (Fletcher |
| | | | | factor) |
| 53b | CG56155-02 | 401 | 402 | Plasma kallikrein |
| | | | | precursor (EC 3.4.21.34) |
| | | | | (Plasma prekallikrein) |
| | | | | (Kininogenin) (Fletcher |
| | | | | factor) |
| 53c | CG56155-03 | 403 | 404 | Plasma kallikrein |
| | | | | precursor (EC 3.4.21.34) |
| | | | | (Plasma prekallikrein) |
| | | | | (Kininogenin) (Fletcher |
| | | | | factor) |
| 54a | CG57191-01 | 405 | 406 | Retinal short-chain |
| | | | | dehydrogenase/reductase |
| | | | | RETSDR1 |
| 54b | CG57191-03 | 407 | 408 | Retinal short-chain |
| | | | | dehydrogenase/reductase |
| | | | | RETSDR1 |
| 54c | CG57191-02 | 409 | 410 | Retinal short-chain |
| | | | | dehydrogenase/reductase |
| | | | | RETSDR1 |
| 55a | CG59595-01 | 411 | 412 | Ribonuclease 6 precursor |
| 55b | 169728691 | 413 | 414 | Ribonuclease 6 precursor |
| 55c | 169728707 | 415 | 416 | Ribonuclease 6 precursor |
| 55d | 169728746 | 417 | 418 | Ribonuclease 6 precursor |
| 55e | CG59595-02 | 419 | 420 | Ribonuclease 6 precursor |
| 55f | CG59595-03 | 421 | 422 | Ribonuclease 6 precursor |
| 55g | CG59595-04 | 423 | 424 | Ribonuclease 6 precursor |
| 55h | CG59595-05 | 425 | 426 | Ribonuclease 6 precursor |
| 56a | CG92142-01 | 427 | 428 | Glycerol-3-phosphate |
| | | | | acyltransferase, |
| | | | | mitochondrial precursor |
| 56b | CG92142-02 | 429 | 430 | Glycerol-3-phosphate |
| | | | | acyltransferase, |
| | | | | mitochondrial precursor |
| 57a | CG95765-01 | 431 | 432 | Hypothetical protein |
| 57b | CG95765-02 | 433 | 434 | Hypothetical protein |
| 58a | CG97178-01 | 435 | 436 | Tryptophan 2,3- |
| | | | | dioxygenase (EC |
| | | | | 1.13.11.11) (Tryptophan |
| | | | | pyrrolase) |
| | | | | (Tryptophanase) |
| | | | | (Tryptophan oxygenase) |
| | | | | (Tryptamin 2,3- |
| | | | | dioxygenase) (TRPO) |
| 58b | 275481043 | 437 | 438 | Tryptophan 2,3- |
| | | | | dioxygenase (EC |
| | | | | 1.13.11.11) (Tryptophan |
| | | | | pyrrolase) |
| | | | | (Tryptophanase) |
| | | | | (Tryptophan oxygenase) |
| | | | | (Tryptamin 2,3- |
| | | | | dioxygenase) (TRPO) |
| 58c | 275481043 | 439 | 440 | Diamine acetyltransferase |
| | | | | (EC 2.3.1.57) |
| | | | | (Spermidine/spermine |
| | | | | N(1)- acetyltransferase) |
| | | | | (SSAT) (Putrescine |
| | | | | acetyltransferase) |
| 59a | CG98102-01 | 441 | 442 | Diamine acetyltransferase |
| | | | | (EC 2.3.1.57) |
| | | | | (Spermidine/spermine |
| | | | | N(1)- acetyltransferase) |
| | | | | (SSAT) (Putrescine |
| | | | | acetyltransferase) |
| 59b | CG98102-03 | 443 | 444 | Diamine acetyltransferase |
| | | | | (EC 2.3.1.57) |
| | | | | (Spermidine/spermine |
| | | | | N(1)- acetyltransferase) |
| | | | | (SSAT) (Putrescine |
| | | | | acetyltransferase) |
| 59c | CG98102-02 | 445 | 446 | Diamine acetyltransferase |
| | | | | (EC 2.3.1.57) |
| | | | | (Spermidine/spermine |
| | | | | N(1)- acetyltransferase) |
| | | | | (SSAT) (Putrescine |
| | | | | acetyltransferase) |
| 59d | CG98102-04 | 447 | 448 | Diamine acetyltransferase |
| | | | | (EC 2.3.1.57) |
| | | | | (Spermidine/spermine |
| | | | | N(1)- acetyltransferase) |
| | | | | (SSAT) (Putrescine |
| | | | | acetyltransferase) |
| 59e | CG98102-05 | 449 | 450 | Diamine acetyltransferase |
| | | | | (EC 2.3.1.57) |
| | | | | (Spermidine/spermine |
| | | | | N(1)- acetyltransferase) |
| | | | | (SSAT) (Putrescine |
| | | | | acetyltransferase) |
| 59f | CG98102-06 | 451 | 452 | Diamine acetyltransferase |
| | | | | (EC 2.3.1.57) |
| | | | | (Spermidine/spermine |
| | | | | N(1)- acetyltransferase) |
| | | | | (SSAT) (Putrescine |
| | | | | acetyltransferase) |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to: e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias,] the metabolic syndrome X and wasting disorders associated with chronic diseases and various cancers, as well as conditions such as transplantation and fertility.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g. detection of a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 226; (b) a variant of a mature form of the amino acid sequence selected from the group consisting of SEQ ID NO: 2n, wherein n is an integer between 1 and 226, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 226; (d) a variant of the amino acid sequence selected from the group consisting of SEQ ID NO:2n, wherein n is an integer between 1 and 226 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 226; (b) a variant of a mature form of the amino acid sequence selected from the group consisting of SEQ ID NO: 2n, wherein n is an integer between 1 and 226 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 226; (d) a variant of the amino acid sequence selected from the group consisting of SEQ ID NO: 2n, wherein n is an integer between 1 and 226, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 226 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 226; (b) a nucleotide sequence wherein one or more nucleotides in the nucleotide sequence selected from the group consisting of SEQ ID NO: 2n−1, wherein n is an integer between 1 and 226 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 226; and (d) a nucleic acid fragment wherein one or more nucleotides in the nucleotide sequence selected from the group consisting of SEQ ID NO: 2n−1, wherein n is an integer between 1 and 226 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb; 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO:2n−1, wherein n is an integer between 1 and 226, or a complement of this nucleotide sequence, can be isolated using standard molecular biology techniques and the sequence information provided herein. Using all or a portion of the nucleic acid sequence of SEQ ID NO:2n−1, wherein n is an integer between 1 and 226, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, NY, 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n−1, wherein n is an integer between 1 and 226, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n−1, wherein n is an integer between 1 and 226, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n−1, wherein n is an integer between 1 and 226, is one that is sufficiently complementary to the nucleotide sequence of SEQ ID NO:2n−1, wherein n is an integer between 1 and 226, that it can hydrogen bond with few or no mismatches to the nucleotide sequence shown in SEQ, ID NO:2n−1, wherein n is an integer between 1 and 226, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1, wherein n is an integer between 1 and 226, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1 and 226; or an anti-sense strand nucleotide sequence of SEQ ID NO:2n−1, wherein n is an integer between 1 and 226; or of a naturally occurring mutant of SEQ ID NO:2n−1, wherein n is an integer between 1 and 226.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1 and 226, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO:2n−1, wherein n is an integer between 1 and 226, due to degeneracy of the genetic code and thus encode the same NOVX proteins as that encoded by the nucleotide sequences of SEQ ID NO:2n−1, wherein n is an integer between 1 and 226. In another embodiment, an isolated nucleic acid molecule of the invention has a nucleotide sequence encoding a protein having an amino acid sequence of SEQ ID NO:2n, wherein n is an integer between I and 226.

In addition to the human NOVX nucleotide sequences of SEQ ID NO:2n−1, wherein n is an integer between 1 and 226, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n−1, wherein n is an integer between 1 and 226, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1 and 226. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO:2n−1, wherein n is an integer between 1 and 226, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1 and 226, or fragments, analogs or derivatives thereof, under conditions of moderate stringency is provided. A non-limiting example of moderate stringency hybridization conditions are hybridization in 6×SSC, 5×Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO:2n−1, wherein n is an integer between 1 and 226, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1 and 226, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 226. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n−1, wherein n is an integer between 1 and 226, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO:2n, wherein n is an integer between 1 and 226. Preferably, the protein encoded by the nucleic acid molecule is at least about 60% homologous to SEQ ID NO:2n, wherein n is an integer between 1 and 226; more preferably at least about 70% homologous to SEQ ID NO:2n, wherein n is an integer between 1 and 226; still more preferably at least about 80% homologous to SEQ ID NO:2n, wherein n is an integer between 1 and 226; even more preferably at least about 90% homologous to SEQ ID NO:2n, wherein n is an integer between 1 and 226; and most preferably at least about 95% homologous to SEQ ID NO:2n, wherein n is an integer between 1 and 226.

An isolated nucleic acid molecule encoding a NOVX protein homologous to the protein of SEQ ID NO:2n, wherein n is an integer between 1 and 226, can be created by introducing one or more nucleotide substitutions, additions or deletions into the nucleotide sequence of SEQ ID NO:2n−1, wherein n is an integer between 1 and 226, such that one or more amino acid substitutions, additions or deletions are introduced into the encoded protein.

Mutations can be introduced any one of SEQ ID NO:2n−1, wherein n is an integer between 1 and 226, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n−1, wherein n is an integer between 1 and 226, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt anti sense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N 19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N 19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX) phenotype in the treated subject sample. The NOVX- phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2×proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n−1, wherein n is an integer between 1 and 226, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 226, or antisense nucleic acids complementary to a NOVX nucleic acid sequence of SEQ ID NO:2n−1, wherein n is an integer between 1 and 226, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl)uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n−1, wherein n is an integer between 1 and 226). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. Bio Techniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1 and 226. The invention also includes a mutant or variant protein any of whose residues may be changed from the corresponding residues shown in any one of SEQ ID NO:2n, wherein n is an integer between 1 and 226, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one. embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 226) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 226. In other embodiments, the NOVX protein is substantially homologous to SEQ ID NO:2n, wherein n is an integer between 1 and 226, and retains the functional activity of the protein of SEQ ID NO:2n, wherein n is an integer between 1 and 226, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 226, and retains the functional activity of the NOVX proteins of SEQ ID NO:2n, wherein n is an integer between 1 and 226.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1 and 226.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 226, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 226, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH 1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al ., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT ™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 1 id (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec 1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NO:2n−1, wherein n is an integer between 1 and 226, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n−1, wherein n is an integer between 1 and 226), but more preferably, is a non-human homologue of a human NOVX gene. For example, a mouse homologue of human NOVX gene of SEQ ID NO:2n−1, wherein n is an integer between 1 and 226, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g. NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO:2n−1, wherein n is an integer between 1 and 226, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n−1, wherein n is an integer between 1 and 226, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n−1, wherein n is an integer between 1 and 226, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the pre-administration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

Polynucleotide and Polypeptide Sequences, and Homology Data

The NOV1 clone was analyzed, and the nucleotide and encoded polypeptide sequences are shown in Table 1A.

TABLE 1A
NOV1 Sequence Analysis

| | |
|---|---|
| | SEQ ID NO: 1 2673 bp |
| NOV1a, | GGATCCCAGTGGCCCGGCGTGCTCGGCTCCCACAGGCCTGCAGCCAGCATCGCACCGA |
| CG101683-01 | |
| DNA Sequence | ACCTTCGGGGGGCCGCGGCTGGAGCGCTCGGCCGGCGTGGGAGCGCAAGGCCGCAGAT |
| | GCAATCTTCTTACCGCGAAGAAGCCAGGGGAATAGGTAGCCACATCTTGTTTGCAGAT |
| | AAGAAAGGAAGCTAACGCAGTATCTGCAAAGCCAGGAGTCTGACTCAGTACTTTTCTC |
| | ACTCATGCATACAAGCAGCTAAAAATGACACAGCTTATTTACCATGCCCCTGACACTG |
| | CACTGAGCACTTTATGAGCTTGAACTCTGTTAATCTCACGACCACCTCATGAGACTCT |
| | CCAGAAAGAGCAACAGTAATGGAGTACATGAGCACTGGAAGTGACAATAAAGAAGAGA |
| | TTGATTTATTAATTAAACATTTAAATGTGTCTGATGTAATAGACATTATGGAAAATCT |
| | TTATGCAAGTGAAGAGCCAGCAGTTTATGAACCCAGTCTAATGACCATGTGTCAAGAC |
| | AGTAATCAAAACGATGAGCGTTCTAAGTCTCTGCTGCTTAGTGGCCAAGAGGTACCAT |
| | GGTTGTCATCAGTCAGATATGGAACTGTGGAGGATTTGCTTGCTTTTGCAAACCATAT |
| | ATCCAACACTGCAAAGCATTTTTATGGACAACGACCACAGGAATCTGGAATTTTATTA |
| | AACATGGTCATCACTCCCCAAAATGGACGTTACCAAATAGATTCCGATGTTCTCCTGA |
| | TCCCCTGGAAGCTGACTTACAGGAATATTGGTTCTGATTTTATTCCTCGGGGCGCCTT |
| | TGGAAAGGTATACTTGGCTCAAGATATAAAGACGAAGAAAAGAATGGCGTGTAAACTG |
| | ATCCCAGTAGATCAATTTAAGCCATCTGATGTGGAAATTCAGGCTTGCTTCCGGCACG |
| | AGAACATCGCAGAGCTGTATGGCGCAGTCCTGTGGGGTGAAACTGTCCATCTCTTTAT |
| | GGAAGCAGGCGAGGGAGGGTCTGTTCTGGAGAAACTGGAGAGCTGTGGACCAATGAGA |
| | GAATTTGAAATTATTTGGGTGACAAAGCATGTTCTCAAGGGACTTGATTTTCTACACT |
| | CAAAGAAAGTGATCCATCATCATATTAAACCTAGCAACATTGTTTTCATGTCCACAAA |
| | AGCTGTTTTGGTGGATTTTGGCCTAAGTGTTCAAATGACCGAAGATGTCTATTTTCCT |
| | AAGCACCTCCGAGGAACAGAGATTTACATGAGCCCAGAGGTCATCCTGTGCAGGGGCC |
| | ATTCAACCAAAGCAGACATCTACAGCCTGGGGCCCACGCTCATCCACATGCAGACGGG |
| | CACCCCACCCTGGGTGAAGCGCTACCCTCGCTCAGCCTATCCCTCCTACCTGTACATA |
| | ATCCACAAGCAACCACCTCCACTGGAAGACATTGCAGATGACTCCAGTCCAGGGATGA |
| | GAGAGCTGATAGAAGCTTCCCTGGAGAGAAACCCCAATCACCGCCCAAGAGCCGCAGA |
| | CCTACTAAAACATGAGGCCCTGAACCCGCCCAGAGAGGATCAGCCACGCTGTACGAGT |
| | CTGGACTCTGCCCTCTTGGAGCGCAAGAGGCTGCTGAGTAGGAAGGAGCTGGAACTTC |
| | CTGAGAACATTGCTGATTCTTCGTGCACAGGAAGCACCGAGGAATCTGAGATGCTCAA |
| | GAGGCAACGCTCTCTCTACATCGACCTCGGCGCTCTGGCTGGCTACTTCAATCTTGTT |
| | CGGGGACCACCAACGCTTGAATATGGCTGAAGGATGCCATGTTTGCCTCTAAATTAAG |
| | ACAGCATTGATCTCCTGGAGGCTGGTTCTGCTGCCTCTACACAGGGGCCCGTTACAGT |
| | GAATGGTGCCATTTTCGAAGGAGCAGTGTGACCTCCTGTGACCCATGAATGTGCCTCC |
| | AAGCGGCCCTGTGTGTTTGACATGTGAAGCTATTTGATATGCACCAGGTCTCAAGGTT |
| | CTCATTTCTCAGGTGACGTGATTCTAAGGCAGGAATTTGAGAGTTCACAGAAGGATCG |
| | TGTCTGCTGACTGTTTCATTCACTGTGCACTTTGCTCAAAATTTTAAAAATACCAATC |
| | ACAAGGATAATAGAGTAGCCTAAAATTACTATTCTTGGTTCTTATTTAAGTATGCAAT |
| | ATTCATTTTACTCAGAATAGCCTGTTTTGTGTATATTGGTGTATATTATATAACTCTT |
| | TGAGCCTTTATTGGTAAATTCTGGTATACATTGAATTCATTATAATTTGGGTGACTAG |
| | AACAACTTGAAGATTGTAGCAATAAGCTGGACTAGTGTCCTAAAAATGGCTAACTGAT |
| | GAATTAGAACCCATCTGACAGACGGCCACTAGTGACAGTTTCTTTTGTGTTCCTATGG |
| | AAACATTTTATACTGTACATGCTATGCTGAAGACATTCAAAACGTGATGTTTTGAATG |
| | TGGATAAAACTCTGTAAACCACATAATTTTGTACATCCAAGGATGAGGTGTGACCTTT |
| | AAGAAAAATGAAAACTTTTGTAAATTATTGATGATTTTGTAATTCTTATGACTAAATT |
| | TTCTTTTAAGCATTTGTATATTAAAATAGCATACTGTGTATGTTTTATATCAAATGCC |
| | TTCATGAATCTTTCATACATATATATATTTGTAACATGTAAAGTATGTGAGTAGTCTT |
| | ATGTAAAGTATGTTTTTACATTATGCAAATAAAACCCAATACTTTTGTCCAATGTGGT |
| | TGGTCAAATCAACTGAATAAATTCAGTATTTTGCCTT |
| | ORF Start: ATG at 367 ORF Stop: TGA at 1768 |
| | SEQ ID NO: 2 467 aa MW at 52896.9kD |
| NOV1a, | MEYMSTGSDNKEEIDLLIKHLNVSDVIDIMENLYASEEPAVYEPSLMTMCQDSNQNDE |
| CG101683-01 | |
| Protein Sequence | RSKSLLLSGQEVPWLSSVRYGTVEDLLAFANHISNTAKHFYGQRPQESGILLNMVITP |
| | QNGRYQIDSDVLLIPWKLTYRNIGSDFIPRGAFGKVYLAQDIKTKKRMACKLIPVDQF |
| | KPSDVEIQACFRHENIAELYGAVLWGETVHLFMEAGEGGSVLEKLESCGPMREFEIIW |
| | VTKHVLKGLDFLHSKKVIHHDIKPSNIVFMSTKAVLVDFGLSVQMTEDVYFPKDLRGT |
| | EIYMSPEVILCRGHSTKADIYSLGATLIHMQTGTPPWVKRYPRSAYPSYLYIIHKQAP |
| | PLEDIADDCSPGMRELIEASLERNPNHRPRAADLLKHEALNPPREDQPRCTSLDSALL |
| | ERKRLLSRKELELPENIADSSCTGSTEESEMLKRQRSLYIDLGALAGYFNLVRGPPTL |
| | SEQ ID NO: 3 1425 bp |
| NOV1b, | ACCATGGAGTACATGAGCACTGGAAGTGACAATAAAGAAGAGATTGATTTATTAATTA |
| 248490507 | |
| DNA Sequence | AACATTTAAATGTGTCTGATGTAATAGACATTATGGAAAATCTTTATGCAAGTGAAGA |
| | CCCAGCAGTTTATGAACCCAGTCTAATGACCATGTGTCAAGACAGTAATCAAAACGAT |
| | GAGCGTTCTAAGTCTCTGCTGCTTAGTGGCCAAGAGGTACCATGGTTGTCATCAGTCA |
| | GATACGGAACTGTGGAGGATTTGCTTGCTTTTGCAAACCATATATCCAACACTGCAAA |
| | GCATTTTTATGGACAACGACCACAGGAATCTGGAATTTTATTAAACATGGTCATCACT |
| | CCCCAAAATGGACGTTACCAAATAGATTCCGATGTTCTCCTGATCCCCTGGAAGCTGA |
| | CTTACAGGAATATTGGTTCTGATTTTATTCCTCGGGGCGCCTTTGGAAAGGTATACTT |
| | GGCACAAGATATAAAGACGAAGAAAAGAATGGCGTGTAAACTGATCCCAGTAGATCAA |
| | TTTAAGCCATCTGATGTGGAAATCCAGGCTTGCTTCCGGCACGAGAACATCGCAGAGC |
| | TGTATGGCGCAGTCCTGTGGGGTGAAACTGTCCATCTCTTTATGGAAGCAGGCGAGGG |
| | AGGGTCTGTTCTGGAGAAACTGGAGAGCTGTGCACCAATGAGAGAATTTGAAATTATT |
| | TGGGTGACAAAGCATGTTCTCAAGGGACTTGATTTTCTACACTCAAAGAAAGTGATCC |
| | ATCATGATATTAAACCTAGCAACATTGTTTTCATGTCCACAAAAGCTGTTTTGGTGGA |
| | TTTTGGCCTAAGTGTTCAAATGACCGAAGATGTCTATTTTCCTAAGGACCTCCGAGGA |
| | ACAGAGATTTACATGAGCCCAGAGGTCATCCTGTGCAGGGGCCATTCAACCAAAGCAG |
| | ACATCTACAGCCTGGGGGCCACGCTCATCCACATGCAGACGCGCACCCCACCCTGGGT |
| | GAAGCGCTACCCTCGCTCAGCCTATCCCTCCTACCTGTACATAATCCACAAGCAAGCA |
| | CCTCCACTGGAAGACATTGCAGATGACTGCAGTCCAGGGATGAGAGAGCTCATACAAG |
| | CTTCCCTGGAGAGAAACCCCAATCACCGCCCAAGAGCCGCAGACCTACTAAAACATGA |
| | GGCCCTGAACCCGCCCAGAGAGGATCAGCCACGCTGTCAGAGTCTGGACTCTGCCCTC |
| | TTGGAGCGCAAGAGGCTGCTGAGTAGGAAGGAGCTGGAACTTCCTGAGAACATTGCTG |
| | ATTCTTCGTCCACAGGAAGCACCGAGGAATCTGAGATGCTCAAGAGGCAACGCTCTCT |
| | CTACATCGACCTCGGCGCTCTGGCTGGCTACTTCAATCTTGTTCGGGCACCACCAACG |
| | CTTGAATATGGCCATCATCACCACCATCACTGA |
| | ORF Start: at 1 ORF Stop: TGA at 1423 |
| | SEQ ID NO: 4 474 aa MW at 53847.9kD |
| NOV1b, | TMEYMSTGSDNKEEIDLLIKHLNVSDVIDIMENLYASEEPAVYEPSLMTMCQDSNQND |
| 248490507 | |
| Protein Sequence | ERSKSLLLSGQEVPWLSSVRYGTVEDLLAFANHISNTAKHFYGQRPQESGILLNMVIT |
| | PQNGRYQIDSDVLLIPWKLTYRNIGSDFIPRGAFGKVYLAQDIKTKKRMACKLIPVDQ |
| | FKPSDVEIQACFRHENIAELYGAVLWGETVHLFMEAGEGGSVLEKLESCGPMREFEII |
| | WVTKHVLKGLDFLHSKKVIHHDIKPSNIVFMSTKAVLVDFGLSVQMTEDVYFPKDLRG |
| | TEIYNSPEVILCRGHSTKADIYSLGATLIHMQTGTPPWVKRYPRSAYPSYLYIIHKQA |
| | PPLEDIADDCSPGMRELIEASLERNPNHRPRAADLLKHEALNPPREDQPRCQSLDSAL |
| | LERKRLLSRKELELPENIADSSCTGSTEESEMLKRQRSLYIDLGALAGYFNLVRGPPT |
| | LEYGHHHHHH |
| | SEQ ID NO: 5 1316 bp |
| NOV1c, | ACGGGATCCACCATGGAGTACATGAGCACTGGAAGTGACAATAAAGAAGAGATTGATT |
| 253174293 | |
| DNA Sequence | TATTAATTAAACATTTAAATGTGTCTGATGTAATAGACATTATGGAAAATCTTTATGC |
| | AAGTGAAGAGCCAGCAGTTTATGAACCCAGTCTAATGACCATCTGTCAAGACAGTAAT |
| | CAAAACGATGAGCGTTCTAAGTCTCTGCTGCTTAGTGGCCAAGAGGTACCATGGTTGT |
| | CATCAGTCAGATACGGAACTGTGGAGGATTTGCTTGCTTTTGCAAACCATATATCCAA |
| | CACTGCAAAGCATTTTTATGGACAACGACCACAGGAATCTGGAATTTTATTAAACATG |
| | GTCATCACTCCCCAAAATGGACGTTACCAAATAGATTCCGATGTTCTCCTGATCCCCT |
| | GGAAGCTGACTTACAGGAATATTGGTTCTGATTTTATTCCTCGGGGCGCCTTTGGAAA |
| | GGTATACTTGGCACAAGATATAAAGACGAAGAAAAGAATGGCGTGTAAACTGATCCCA |
| | GTAGATCAATTTAAGCCATCTGATGTGGAAATCCAGGCTTGCTTCCGGCACGAGAACA |
| | TCGCAGAGCTGTATGGCGCAGTCCTGTGGGGTGAAACTGTCCATCTCTTTATGGAAGC |
| | AGGCGAGGGAGGGTCTGTTCTGGAGAAACTGGAGAGCTGTGGACCAATGAGAGAATTT |
| | GAAATTATTTGGGTGACAAAGCATGTTCTCAAGGGACTTGATTTTCTACACTCAAAGA |
| | AAGTGATCCATCATGATATTAAACCTAGCAACATTGTTTTCATGTCCACAAAAGCTGT |
| | TTTGGTGGATTTTGGCCTAAGTGTTCAAATGACCGAAGATGTCTATTTTCCTAAGGAC |
| | CTCCGAGGAACAGAGATTTACATGAGCCCAGAGGTCATCCTGTGCAGGGGCCATTCAA |
| | CCAAAGCAGACATCTACAGCCTGGGGGCCACGCTCATCCACATGCAGACGGGCACCCC |
| | ACCCTGGGTGAAGCGCTACCCTCGCTCAGCCTATCCCTCCTACCTGTACATAATCCAC |
| | AAGCAAGCACCTCCACTGGAAGACATTGCAGATGACTGCAGTCCAGGGATGAGAGAGC |
| | TGATAGAAGCTTCCCTGGAGAGAAACCCCAATCACCGCCCAAGAGCCGCAGACCTACT |
| | AAAACATGAGGCCCTGAACCCGCCCAGAGAGGATCAGCCACGCTGTCAGAGTCTGGAC |
| | TCTGCCCTCTTGGAGCGCAAGAGGCTGCTGAGTAGGAAGGAGCTGGAACTTCCTGAGA |
| | ACATTGCTCATCATCACCACCATCACTGAGCGGCCGCAAG |
| | ORF Start: at 1 ORF Stop: TGA at 1303 |
| | SEQ ID NO: 6 434 aa MW at 49384.9 kD |
| NOV1c, | TGSTMEYNSTGSDNKEEIDLLIKHLNVSDVIDIMENLYASEEPAVYEPSLMTMCQDSN |
| 253174293 | |
| Protein Sequence | QNDERSKSLLLSGQEVPWLSSVRYGTVEDLLAFANHISNTAKHFYGQRPQESGILLNM |
| | VITPQNGRYQIDSDVLLIPWKLTYRNIGSDFIPRGAFGKVYLAQDIKTKKRMACKLIP |
| | VDQFKPSDVEIQACFRHENIAELYGAVLWGETVHLFMEAGEGGSVLEKLESCGPMREF |
| | EIIWVTKHVLKGLDFLHSKKVIHHDIKPSNIVFMSTKAVLVDFGLSVQMTEDVYFPKD |
| | LRGTEIYMSPEVILCRGHSTKADIYSLGATLIHMQTGTPPWVKRYPRSAYPSYLYIIH |
| | KQAPPLEDIADDCSPGMRELIEASLERNPNHRPRAADLLKHEALNPPREDQPRCQSLD |
| | SALLERKRLLSRKELELPENIAHHHHHH |
| | SEQ ID NO: 7 1407 bp |
| NOV1d, | ACCATGGAGTACATGAGCACTGGAAGTGACAATAAAGAAGAGATTGATTTATTAATTA |
| 248490584 | |
| DNA Sequence | AACATTTAAATGTGTCTGATGTAATAGACATTATGGAAAATCTTTATGCAAGTGAAGA |
| | GCCAGCAGTTTATGAACCCAGTCTAATGACCATGTGTCAAGACAGTAATCAAACGAT |
| | GAGCGTTCTAAGTCTCTGCTGCTTAGTGGCCAAGAGGTACCATGGTTGTCATCAGTCA |
| | GATACGGAACTGTGGAGGATTTGCTTGCTTTTGCAAACCATATATCCAACACTGCAAA |
| | GCATTTTTATGGACAACGACCACAGGAATCTGGAATTTTATTAAACATCGTCATCACT |
| | CCCCAAAATGGACGTTACCAAATAGATTCCGATGTTCTCCTGATCCCCTGGAAGCTGA |
| | CTTACAGGAATATTGGTTCTGATTTTATTCCTCGGGGCGCCTTTGGAAAGGTATACTT |
| | GGCACAAGATATAAAGACGAAGAAAAGAATGGCGTGTAAACTGATCCCAGTAGATCAA |
| | TTTAAGCCATCTGATGTGGAAATCCAGGCTTGCTTCCGGCACGAGAACATCCCAGAGC |
| | TGTATGGCGCAGTCCTGTGGGGTGAAACTGTCCATCTCTTTATGGAAGCAGGCGAGGG |
| | AGGGTCTGTTCTGGAGAAACTGGAGAGCTGTGGACCAATGAGAGAATTTGAAATTATT |
| | TGGGTGACAAAGCATGTTCTCAAGGGACTTGATTTTCTACACTCAAAGAAAGTGATCC |
| | ATCATGATATTAAACCTAGCAACATTGTTTTCATGTCCACAAAAGCTGTTTTGGTGGA |
| | TTTTGGCCTAAGTGTTCAAATGACCGAAGATGTCTATTTTCCTAAGGACCTCCGAGGA |
| | ACAGAGATTTACATGAGCCCAGAGGTCATCCTGTGCAGGGGCCATTCAACCAAAGCAG |
| | ACATCTACAGCCTGGGGGCCACGCTCATCCACATGCAGACGGGCACCCCACCCTGGGT |
| | GAAGCGCTACCCTCGCTCAGCCTATCCCTCCTACCTGTACATAATCCACAACCAAGCA |
| | CCTCCACTGGAAGACATTGCAGATGACTGCAGTCCAGGGATCAGAGAGCTGATAGAAG |
| | CTTCCCTGGAGAGAAACCCCAATCACCGCCCAAGAGCCGCAGACCTACTAAAACATGA |
| | GGCCCTGAACCCGCCCAGAGAGGATCAGCCACGCTGTCAGAGTCTGGACTCTGCCCTC |
| | TTGGAGCGCAAGAGGCTGCTGAGTAGGAAGGAGCTGGAACTTCCTGAGAACATTGCTG |
| | ATTCTTCGTGCACAGGAAGCACCGAGGAATCTGAGATGCTCAAGAGGCAACGCTCTCT |
| | CTACATCGACCTCGGCGCTCTGGCTGGCTACTTCAATCTTGTTCGGGGACCACCAACG |
| | CTTGAATATGGCTGA |
| | ORF Start: at 1 ORF Stop: TGA at 1405 |
| | SEQ ID NO: 8 468 aa MW at 53025.0 kD |
| NOV1d, | TMEYMSTGSDNKEEIDLLIKHLNVSDVIDIMENLYASEEPAVYEPSLMTMCQDSNQND |
| 248490584 | |
| Protein Sequence | ERSKSLLLSCQEVPWLSSVRYGTVEDLLAFANHISNTAKHFYGQRPQESGILLNNVIT |
| | PQNGRYQIDSDVLLIPWKLTYRNIGSDFIPRGAFGKVYLAQDIKTKKRMACKLIPVDQ |
| | FKPSDVEIQACFRHENIAELYGAVLWGETVHLFMEAGEGGSVLEKLESCGPMREFEII |
| | WVTKHVLKGLDFLHSKKVIHHDIKPSNIVFMSTKAVLVDFGLSVQMTEDVYFPKDLRG |
| | TEIYMSPEVILCRGHSTKADIYSLGATLIHMQTGTPPWVKRYPRSAYPSYLYIIHKQA |
| | PPLEDIADDCSPGMRELIEASLERNPNHRPRAADLLKHEALNPPREDQPRCQSLDSAL |
| | LERKRLLSRKELELPEMIADSSCTGSTEESEMLKRQRSLYIDLGALAGYFNLVRGPPT |
| | LEYG |
| | SEQ ID NO: 9 1448 bp |
| NOV1e, | ACGGGATCCACCATGGGACATCATCACCACCATCACGAGTACATGAGCACTGGAAGTG |
| 258054391 | |
| DNA Sequence | ACAATAAAGAAGAGATTGATTTATTAATTAAACATTTAAATGTGTCTGATGTAATAGA |
| | CATTATGGAAAATCTTTATGCAAGTGAAGAGCCAGCAGTTTATGAACCCAGTCTAATG |
| | ACCATGTGTCAAGACAGTAATCAAAACGATGAGCGTTCTAAGTCTCTGCTGCTTAGTG |
| | GCCAAGAGGTACCATGGTTGTCATCAGTCAGATACGGAACTGTGGAGGATTTGCTTGC |
| | TTTTGCAAACCATATATCCAACACTGCAAAGCATTTTTATGGACAACGACCACAGGAA |
| | TCTGGAATTTTATTAAACATGGTCATCACTCCCCAAAATGGACGTTACCAAATAGATT |
| | CCGATGTTCTCCTGATCCCCTGGAAGCTGACTTACAGGAATATTGGTTCTGATTTTAT |
| | TCCTCGGGGCGCCTTTGGAAAGGTATACTTGGCACAAGATATAAAGACGAAGAAAAGA |
| | ATGGCGTGTAAACTGATCCCAGTAGATCAATTTAAGCCATCTGATGTGGAAATCCAGG |
| | CTTGCTTCCGGCACGAGAACATCGCAGAGCTGTATGGCGCAGTCCTGTGGGGTGAAAC |
| | TGTCCATCTCTTTATGGAAGCAGGCGAGGGAGGGTCTGTTCTGGAGAAACTGGAGAGC |
| | TGTGGACCAATGAGAGAATTTGAAATTATTTGGGTGACAAAGCATGTTCTCAAGGGAC |
| | TTGATTTTCTACACTCAAAGAAAGTGATCCATCATGATATTAAACCTAGCAACATTGT |
| | TTTCATGTCCACAAAAGCTGTTTTGGTGGATTTTGGCCTAAGTGTTCAAATGACCGAA |
| | GATGTCTATTTTCCTAAGGACCTCCGAGGAACAGAGATTTACATGAGCCCAGAGGTCA |
| | TCCTGTGCAGGGGCCATTCAACCAAAGCAGACATCTACAGCCTGGGGGCCACGCTCAT |
| | CCACATGCAGACGGGCACCCCACCCTGGGTGAAGCGCTACCCTCGCTCAGCCTATCCC |
| | TCCTACCTGTACATAATCCACAAGCAAGCACCTCCACTGGAAGACATTGCAGATGACT |
| | GCAGTCCAGGGATGAGAGAGCTGATAGAAGCTTCCCTGGAGAGAAACCCCAATCACCG |
| | CCCAAGAGCCGCAGACCTACTAAAACATGAGGCCCTGAACCCGCCCAGAGAGGATCAG |
| | CCACGCTGTCAGAGTCTGGACTCTGCCCTCTTGGAGCGCAAGAGGCTGCTGAGTAGGA |
| | AGGAGCTGGAACTTCCTGAGAACATTGCTGATTCTTCGTGCACAGGAAGCACCGAGGA |
| | ATCTGAGATGCTCAAGAGGCAACGCTCTCTCTACATCGACCTCGGCGCTCTGGCTGGC |
| | TACTTCAATCTTGTTCGGGGACCACCAACGCTTGAATATGGCTGAGCGGCCGCAAG |
| | ORF Start: at 1 ORF Stop: TGA at 1435 |
| | SEQ ID NO: 10 1478 aa MW at 54150.2 kD |
| NOV1e, | TGSTMGHHHHHHEYMSTGSDNKEEIDLLIKHLNVSDVIDIMENLYASEEPAVYEPSLM |
| 258054391 | |
| Protein Sequence | TMCQDSNQNDERSKSLLLSGQEVPWLSSVRYGTVEDLLAFANHISNTAKHFYGQRPQE |
| | SGILLNMVITPQNGRYQIDSDVLLIPWKLTYRNIGSDFIPRGAFGKVYLAQDIKTKKR |
| | MACKLIPVDQFKPSDVEIQACFRHENIAELYGAVLWGETVHLFMEAGEGGSVLEKLES |
| | CGPMREFEIIWVTKHVLKGLDFLHSKKVIHHDIKPSNIVFMSTKAVLVDFGLSVQMTE |
| | DVYFPKDLRGTEIYMSPEVILCRGHSTKADIYSLGATLIHMQTGTPPWVKRYPRSAYP |
| | SYLYIIHKQAPPLEDIADDCSPGMRELIEASLERNPNHRPRAADLLKHEALNPPREDQ |
| | PRCQSLDSALLERKRLLSRKELELPENIADSSCTGSTEESEMLKRQRSLYIDLGALAG |
| | YFNLVRGPPTLEYG |
| | SEQ ID NO: 11 1278 bp |
| NOV1f, | ACCATGGAGTACATGAGCACTGGAAGTGACAATAAAGAAGAGATTGATTTATTAATTA |
| 248494549 | |
| DNA Sequence | AACATTTAAATGTGTCTGATGTAATAGACATTATGGAAAATCTTTATGCAAGTGAAGA |
| | GCCAGCAGTTTATGAACCCAGTCTAATGACCATGTGTCAAGACAGTAATCAAAACGAT |
| | GAGCGTTCTAAGTCTCTGCTGCTTAGTGGCCAAGAGGTACCATGGTTGTCATCAGTCA |
| | GATACGGAACTGTGGAGGATTTGCTTGCTTTTGCAAACCATATATCCAACACTGCAAA |
| | GCATTTTTATGGACAACGACCACAGGAATCTGGAATTTTATTAAACATGGTCATCACT |
| | CCCCAAAATGGACGTTACCAAATAGATTCCGATGTTCTCCTGATCCCCTGGAAGCTGA |
| | CTTACAGGAATATTGGTTCTGATTTTATTCCTCGGGGCGCCTTTGGAAAGGTATACTT |
| | GGCACAAGATATAAAGACGAAGAAAAGAATGGCGTGTAAACTGATCCCAGTAGATCAA |
| | TTTAAGCCATCTGATGTGGAAATCCAGGCTTGCTTCCGGCACGAGAACATCGCAGAGC |
| | TGTATGGCGCAGTCCTGTGGGGTGAAACTGTCCATCTCTTTATGGAAGCAGGCGAGGG |
| | AGGGTCTGTTCTGGAGAAACTGGAGAGCTGTGGACCAATGAGAGAATTTGAAATTATT |
| | TGGGTGACAAAGCATGTTCTCAAGGGACTTGATTTTCTACACTCAAAGAAAGTGATCC |
| | ATCATGATATTAAACCTAGCAACATTGTTTTCATGTCCACAAAAGCTGTTTTGGTGGA |
| | TTTTGGCCTAAGTGTTCAAATGACCGAAGATGTCTATTTTCCTAAGGACCTCCGAGGA |
| | ACAGAGATTTACATGAGCCCAGAGGTCATCCTGTGCAGGGGCCATTCAACCAAAGCAG |
| | ACATCTACAGCCTGGGGGCCACCCTCATCCACATGCAGACGGGCACCCCACCCTGGGT |
| | GAAGCGCTACCCTCGCTCAGCCTATCCCTCCTACCTGTACATAATCCACAAGCAAGCA |
| | CCTCCACTGGAAGACATTGCAGATGACTGCAGTCCAGGGATGAGAGAGCTGATAGAAG |
| | CTTCCCTGGAGAGAAACCCCAATCACCGCCCAAGAGCCGCAGACCTACTAAAACATGA |
| | GGCCCTGAACCCGCCCAGAGAGGATCAGCCACGCTGTCAGAGTCTGGACTCTGCCCTC |
| | TTGGAGCGCAAGAGGCTGCTGAGTAGGAAGGAGCTGGAACTTCCTGAGAACATTGCTT |
| | GA |
| | ORF Start: at 1 ORF Stop: TGA at 1276 |
| | SEQ ID NO: 12 425 aa MW at 48316.8 kD |
| NOV1f | TMEYMSTGSDNKEEIDLLTKHLNVSDVIDIMENLYASEEPAVYEPSLMTMCQDSNQND |
| 248494549 | |
| Protein Sequence | ERSKSLLLSGQEVPWLSSVRYGTVEDLLAFANHISNTAKHFYGQRPQESGILLNMVIT |
| | PQNGRYQIDSDVLLIPWKLTYRNIGSDFIPRGAFGKVYLAQDIKTKKRMACKLIPVDQ |
| | FKPSDVEIQACFRHENIAELYGAVLWGETVHLFMEAGEGGSVLEKLESCGPMREFEII |
| | WVTKHVLKGLDFLHSKKVIHHDIKPSNIVFMSTKAVLVDFGLSVQMTEDVYFPKDLRG |
| | TEIYMSPEVILCRGHSTKADIYSLGATLIHMQTGTPPWVKRYPRSAYPSYLYIIHKQA |
| | PPLEDIADDCSPGMRELIEASLERNPNHRPRAADLLKHEALNPPREDQPRCQSLDSAL |
| | LERKRLLSRKELELPENIA |
| | SEQ ID NO: 13 1327 bp |
| NOV1g, | CCACCATCGGGCGCGGATCCACCATGGGACATCATCACCACCATCACGAGTACATGAG |
| 259741837 | |
| DNA Sequence | CACTGGAAGTGACAATAAAGAAGAGATTGATTTATTAATTAAACATTTAAATGTGTCT |
| | GATGTAATAGACATTATGGAAAATCTTTATGCAAGTGAAGAGCCAGCAGTTTATGAAC |
| | CCAGTCTAATGACCATGTGTCAAGACAGTAATCAAAACGATGAGCGTTCTAAGTCTCT |
| | GCTGCTTAGTGGCCAAGAGGTACCATGGTTGTCATCAGTCAGATACGGAACTGTGGAG |
| | GATTTGCTTGCTTTTGCAAACCATATATCCAACACTGCAAAGCATTTTTATGGACAAC |
| | GACCACAGGAATCTGGAATTTTATTAAACATGGTCATCACTCCCCAAAATGGACGTTA |
| | CCAAATAGATTCCGATGTTCTCCTGATCCCCTGGAAGCTGACTTACAGGAATATTGGT |
| | TCTGATTTTATTCCTCGGGGCGCCTTTGGAAAGGTATACTTGGCACAAGATATAAAGA |
| | CGAAGAAAAGAATGGCGTGTAAACTGATCCCAGTAGATCAATTTAAGCCATCTGATGT |
| | GGAAATCCAGGCTTGCTTCCGGCACGAGAACATCGCAGAGCTGTATGGCGCAGTCCTG |
| | TGGGGTGAAACTGTCCATCTCTTTATGGAAGCAGGCGAGGGAGGGTCTGTTCTGGAGA |
| | AACTGGAGAGCTGTGGACCAATGAGAGAATTTGAAATTATTTGGGTGACAAAGCATGT |
| | TCTCAAGGGACTTGATTTTCTACACTCAAAGAAAGTGATCCATCATGATATTAAACCT |
| | AGCAACATTGTTTTCATGTCCACAAAAGCTGTTTTGGTGGATTTTGGCCTAAGTGTTC |
| | AAATGACCGAAGATGTCTATTTTCCTAAGGACCTCCGACGAACAGAGATTTACATGAG |
| | CCCAGAGGTCATCCTGTGCAGGGGCCATTCAACCAAAGCAGACATCTACAGCCTGGGG |
| | GCCACGCTCATCCACATGCAGACGGGCACCCCACCCTGGGTGAAGCGCTACCCTCGCT |
| | CAGCCTATCCCTCCTACCTGTACATAATCCACAAGCAAGCACCTCCACTGGAAGACAT |
| | TGCAGATCACTGCAGTCCAGGGATGAGAGAGCTGATACAAGCTTCCCTGGAGAGAAAC |
| | CCCAATCACCGCCCAAGAGCCGCAGACCTACTAAAACATGAGGCCCTGAACCCGCCCA |
| | GAGAGGATCAGCCACGCTGTCAGAGTCTGGACTCTGCCCTCTTGGAGCGCAAGAGGCT |
| | GCTGAGTAGGAAGGAGCTGGAACTTCCTGAGAACATTGCTTGAGGCGGCCG |
| | ORF Start: at 3 ORF Stop: TGA at 1317 |
| | SEQ ID NO: 14 438 aa MW at 49768.4 kD |
| NOV1g, | TIGRGSTMGHHHHHHEYMSTGSDNKEEIDLLIKHLNVSDVIDIMENLYASEEPAVYEP |
| 259741837 | |
| Protein Sequence | SLMTMCQDSNQNDERSKSLLLSGQEVPWLSSVRYGTVEDLLAFANHISNTAKHFYGQR |
| | PQESGILLNMVITPQNGRYQIDSDVLLIPWKLTYRNIGSDFIPRGAFGKVYLAQDIKT |
| | KKRMACKLIPVDQFKPSDVEIQACFRHENIAELYGAVLWGETVHLFMEAGEGGSVLEK |
| | LESCGPMREFEIIWVTKHVLKGLDFLHSKKVIHHDIKPSNIVFMSTKAVLVDFGLSVQ |
| | MTEDVYFPKDLRGTEIYMSPEVILCRGHSTKADIYSLGATLIHMQTGTPPWVKRYPRS |
| | AYPSYLYIIHKQAPPLEDIADDCSPGMRELIEASLERNPNHRPRAADLLKHEALNPPR |
| | EDQPRCQSLDSALLERKRLLSRKELELPENIA |
| | SEQ. ID NO: 15 1428 bp |
| NOV1h, | ACCATGGGACATCATCACCACCATCACGAGTACATGAGCACTGGAAGTGACAATAAAG |
| 260480803 | |
| DNA Sequence | AAGAGATTGATTTATTAATTAAACATTTAAATGTGTCTGATGTAATAGACATTATGGA |
| | AAATCTTTATGCAAGTGAAGAGCCAGCAGTTTATGAACCCAGTCTAATGACCATGTGT |
| | CAAGACAGTAATCAAAACGATGAGCGTTCTAAGTCTCTGCTGCTTAGTGGCCAAGAGG |
| | TACCATGGTTGTCATCAGTCAGATACGGAACTGTGGAGGATTTGCTTGCTTTTGCAAA |
| | CCATATATCCAACACTGCAAAGCATTTTTATGGACAACGACCACAGGAATCTGGAATT |
| | TTATTAAACATGGTCATCACTCCCCAAAATGGACGTTACCAAATAGATTCCGATGTTC |
| | TCCTGATCCCCTGGAAGCTGACTTACAGGAATATTGGTTCTGATTTTATTCCTCGGGG |
| | CGCCTTTGGAAAGGTATACTTGGCACAAGATATAAAGACGAAGAAAAGAATGGCGTGT |
| | AAACTGATCCCAGTAGATCAATTTAAGCCATCTGATGTGGAAATCCAGGCTTGCTTCC |
| | GGCACGAGAACATCGCAGAGCTGTATGGCGCAGTCCTGTGGGGTGAAACTGTCCATCT |
| | CTTTATGGAAGCAGGCGAGGGAGGGTCTGTTCTGGAGAAACTGGAGAGCTGTGGACCA |
| | ATGAGAGAATTTGAAATTATTTGGGTGACAAAGCATGTTCTCAAGGGACTTGATTTTC |
| | TACACTCAAAGAAAGTGATCCATCATGATATTAAACCTAGCAACATTGTTTTCATGTC |
| | CACAAAAGCTGTTTTGGTGGATTTTGGCCTAAGTGTTCAAATGACCGAAGATGTCTAT |
| | TTTCCTAAGGACCTCCGAGGAACAGAGATTTACATGAGCCCAGAGGTCATCCTGTGCA |
| | GGGGCCATTCAACCAAAGCACACATCTACAGCCTGGGGGCCACGCTCATCCACATGCA |
| | GACGGGCACCCCACCCTGGGTGAAGCGCTACCCTCGCTCAGCCTATCCCTCCTACCTG |
| | TACATAATCCACAAGCAAGCACCTCCACTGGAAGACATTGCAGATGACTGCAGTCCAG |
| | GGATGAGAGAGCTGATAGAAGCTTCCCTGGAGAGAAACCCCAATCACCGCCCAAGAGC |
| | CGCAGACCTACTAAAACATCAGGCCCTGAACCCGCCCAGAGAGGATCAGCCACGCTGT |
| | CAGAGTCTGGACTCTGCCCTCTTGGAGCGCAAGAGGCTGCTGAGTAGGAAGGAGCTGG |
| | AACTTCCTGAGAACATTGCTGATTCTTCGTCCACAGGAAGCACCGAGGAATCTCAGAT |
| | GCTCAAGAGGCAACCCTCTCTCTACATCGACCTCGGCGCTCTGGCTGGCTACTTCAAT |
| | CTTGTTCGGGGACCACCAACGCTTGAATATGGCTGA |
| | ORF Start: at 1 ORF Stop: TGA at 1426 |
| | SEQ ID NO: 16 475 aa MW at 53904.9kD |
| NOV1h, | TMGHHHHHHEYMSTGSDNKEEIDLLIKHLNVSDVIDIMENLYASEEPAVYEPSLMTMC |
| 260480803 | |
| Protein Sequence | QDSNQNDERSKSLLLSCQEVPWLSSVRYGTVEDLLAFANHISNTAKHFYGQRPQESGI |
| | LLNMVITPQNGRYQIDSDVLLIPWKLTYRNIGSDFIPRGAFGKVYLAQDIKTKKRMAC |
| | KLIPVDQFKPSDVEIQACFRHENIAELYGAVLWGETVHLFMEAGEGGSVLEKLESCGP |
| | MREFEIIWVTKHVLKGLDFLHSKKVIHHDIKPSNIVFMSTKAVLVDFGLSVQMTEDVY |
| | FPKDLRGTEIYMSPEVILCRGHSTKADIYSLGATLIHMQTGTPPWVKRYPRSAYPSYL |
| | YIIHKQAPPLEDIADDCSPGMRELIEASLERNPNHRPRAADLLKHEALNPPREDQPRC |
| | QSLDSALLERKRLLSRKELELPENIADSSCTGSTEESEMLKRQRSLYIDLGALAGYFN |
| | LVRCPPTLEYG |
| | SEQ ID NO: 17 1434 bp |
| NOV1i, | CGCGGATCCACCATGGAGTACATGAGCACTGGAAGTGACAATAAAGAAGAGATTGATT |
| 209983329 | |
| DNA Sequence | TATTAATTAAACATTTAAATGTGTCTGATGTAATAGACATTATGGAAAATCTTTATGC |
| | AAGTGAAGAGCCAGCAGTTTATGAACCCAGTCTAATGACCATGTGTCAACACAGTAAT |
| | CAAAACGATGAGCGTTCTAAGTCTCTGCTGCTTAGTGGCCAAGACGTACCATGGTTGT |
| | CATCACTCAGATACGGAACTGTGGAGGATTTGCTTGCTTTTGCAAACCATATATCCAA |
| | CACTGCAAAGCATTTTTATGGACAACGACCACAGGAATCTGGAATTTTATTAAACATG |
| | GTCATCACTCCCCAAAATGGACGTTACCAAATAGATTCCGATGTTCTCCTGATCCCCT |
| | GGAAGCTGACTTACAGGAATATTGGTTCTGATTTTATTCCTCGGGGCGCCTTTGGAAA |
| | GGTATACTTGGCACAAGATATAAAGACGAAGAAAAGAATGGCGTGTAAACTGATCCCA |
| | GTAGATCAATTTAAGCCATCTGATGTGGAAATCCAGGCTTGCTTCCGGCACGAGAACA |
| | TCGCAGAGCTGTATGGCGCAGTCCTGTGGGGTGAAACTGTCCATCTCTTTATGGAAGC |
| | AGGCGAGGGAGGGTCTGTTCTGGAGAAACTGGAGAGCTGTGGACCAATGAGAGAATTT |
| | GAAATTATTTGGGTGACAAAGCATGTTCTCAAGGGACTTGATTTTCTACACTCAAAGA |
| | AAGTGATCCATCATGATATTAAACCTAGCAACATTGTTTTCATGTCCACAAAAGCTGT |
| | TTTGGTGGATTTTGGCCTAAGTGTTCAAATGACCGAAGATGTCTATTTTCCTAAGGAC |
| | CTCCGAGGAACAGAGATTTACATGAGCCCAGAGGTCATCCTGTGCAGGGGCCATTCAA |
| | CCAAAGCAGACATCTACAGCCTGGGGGCCACGCTCATCCACATGCAGACGGGCACCCC |
| | ACCCTGGGTGAAGCGCTACCCTCGCTCAGCCTATCCCTCCTACCTGTACATAATCCAC |
| | AAGCAAGCACCTCCACTGGAAGACATTGCAGATGACTGCAGTCCAGGGATGAGAGAGC |
| | TGATAGAAGCTTCCCTGGAGAGAAACCCCAATCACCGCCCAAGAGCCGCAGACCTACT |
| | AAAACATGAGGCCCTGAACCCGCCCAGAGAGGATCAGCCACGCTGTCAGAGTCTGGAC |
| | TCTGCCCTCTTGGACCGCAAGAGGCTGCTGAGTAGGAAGGAGCTGGAACTTCCTGAGA |
| | ACATTCCTGATTCTTCGTGCACAGGAAGCACCGAGGAATCTGAGATGCTCAAGAGGCA |
| | ACGCTCTCTCTACATCGACCTCGGCGCTCTGGCTGGCTACTTCAATCTTGTTCGGGGA |
| | CCACCAACCCTTGAATATGGCTGAGCGGCCGCTTTTTTCCTT |
| | ORF Start: at 1 ORF Stop: TGA at 1414 |
| | SEQ ID NO: 18 471 aa MW at 53325.3kD |
| NOV1i | RGSTMEYMSTGSDNKEEIDLLIKHLNVSDVIDIMENLYASEEPAVYEPSLMTMCQDSN |
| 209983329 | |
| Protein Sequence | QNDERSKSLLLSGQEVPWLSSVRYGTVEDLLAFANHISNTAKHFYGQRPQESGILLNM |
| | VITPQNGRYQIDSDVLLIPWKLTYRNIGSDFIPRGAFGKVYLAQDIKTKKRMACKLIP |
| | VDQFKPSDVEIQACFRHENIAELYGAVLWGETVHLFMEAGEGGSVLEKLESCGPMREF |
| | EIIWVTKHVLKGLDFLHSKKVIHHDIKPSNIVFMSTKAVLVDFGLSVQMTEDVYFPKD |
| | LRGTEIYMSPEVILCRGHSTKADIYSLGATLIHMQTGTPPWVKRYPRSAYPSYLYIIH |
| | KQAPPLEDIADDCSPGMRELIEASLERNPNHRPPAADLLKHEALNPPREDQPRCQSLD |
| | SALLERKRLLSRKELELPENIADSSCTGSTEESEMLKRQRSLYIDLGALAGYFNLVRG |
| | PPTLEYG |
| | SEQ ID NO: 19 1772 bp |
| NOV1j, | TGTCGTAACAACTCCGCCCCATTGACGCAAATGGGCGGTAGGCGTGTACGGTGGGAGG |
| 212779055 | |
| DNA Sequence | TCTATATAAGCAGAGCTCTCTGGCTAACTAGAGAACCCACTGCTTACTGGCTTATCGA |
| | AATTAATACGACTCACTATAGGGAGACCCAAGCTGGCTAGCGTTTAAACTTAAGCTTG |
| | GTACCGAGCTCGGATCCACCATGGAGTACATGAGCACTGGAAGTGACAATAAAGAAGA |
| | GATTGATTTATTAATTAAACATTTAAATGTGTCTGATGTAATACACATTATGGAAAAT |
| | CTTTATGCAAGTGAAGAGCCAGCAGTTTATGAACCCAGTCTAATGACCATGTGTCAAG |
| | ACAGTAATCAAAACGATGAGCGTTCTAAGTCTCTGCTGCTTAGTGGCCAAGAGGTACC |
| | ATGGTTGTCATCAGTCAGATACGGAACTGTGGAGGATTTGCTTGCTTTTGCAAACCAT |
| | ATATCCAACACTGCAAAGCATTTTTATGGACAACGACCACAGGAATCTGGAATTTTAT |
| | TAAACATGGTCATCACTCCCCAAAATGGACGTTACCAAATAGATTCCGATGTTCTCCT |
| | GATCCCCTGGAAGCTGACTTACAGGAATATTGGTTCTGATTTTATTCCTCGGGGCGCC |
| | TTTGGAAAGGTATACTTGGCACAAGATATAAAGACGAAGAAAAGAATGGCGTGTAAAC |
| | TGATCCCAGTAGATCAATTTAAGCCATCTGATGTGGAAATCCAGGCTTCCTTCCGGCA |
| | CGAGAACATCGCAGAGCTGTATGGCGCAGTCCTGTGGGGTGAAACTGTCCATCTCTTT |
| | ATGGAAGCAGGCGAGGGAGGGTCTGTTCTGGAGAAACTGGAGAGCTGTGGACCAATGA |
| | GAGAATTTGAAATTATTTGGGTGACAAAGCATGTTCTCAAGGGACTTGATTTTCTACA |
| | CTCAAAGAAAGTGATCCATCATGATATTAAACCTAGCAACATTGTTTTCATGTCCACA |
| | AAAGCTGTTTTGGTGGATTTTGGCCTAAGTGTTCAAATGACCGAAGATGTCTATTTTC |
| | CTAAGGACCTCCGAGGAACAGAGATTTACATGAGCCCACAGGTCATCCTGTGCAGGGG |
| | CCATTCAACCAAAGCAGACATCTACAGCCTGGGGCCCACGCTCATCCACATGCAGACG |
| | GGCACCCCACCCTGGGTGAAGCGCTACCCTCGCTCAGCCTATCCCTCCTACCTGTACA |
| | TAATCCACAAGCAAGCACCTCCACTGGAAGACATTGCAGATGACTGCAGTCCAGGGAT |
| | GAGAGAGCTGATAGAAGCTTCCCTGGAGAGAAACCCCAATCACCGCCCAAGAGCCGCA |
| | GACCTACTAAAACATGAGGCCCTGAACCCGCCCAGAGAGGATCAGCCACGCTGTCAGA |
| | GTCTGGACTCTGCCCTCTTGGAGCGCAAGAGGCTGCTGAGTAGGAAGGAGCTGGAACT |
| | TCCTGAGAACATTGCTGATTCTTCGTGCACAGGAAGCACCGAGGAATCTGAGATGCTC |
| | AAGAGGCAACGCTCTCTCTACATCGACCTCGGCGCTCTGGCTGGCTACTTCAATCTTG |
| | TTCGGGGACCACCAACGCTTGAATATGGCTGAGCGGCCGCTCGAGTCTAGAGGGCCCG |
| | TTTAAACCCGCTGATCAGCCTCGACTGTGCCTTCTAGTTGCCAGCCATCTGTTGTTTG |
| | CCCCTCCCCCGTGCCTTCCTTGACCCTGGAAGGTGCCACTCCCACTGTCCTTTCCTAA |
| | TAAAATGAGGAAATTGCATCGCATTGTCTGAG |
| | ORF Start: at 138 ORF Stop: TGA at 1596 |
| | SEQ ID NO: 20 486 aa MW at 54926.2 kD |
| NOV1j, | GDPSWLAFKLKLGTELCSTMEYMSTGSDNKEEIDLLIKHLNVSDVIDIMENLYASEEP |
| 212779055 | |
| Protein Sequence | AVYEPSLMTMCQDSNQNDERSKSLLLSGQEVPWLSSVRYGTVEDLLAFANHISNTAKH |
| | FYGQRPQESGILLNMVITPQNGRYQIDSDVLLIPWKLTYRNIGSDFIPRGAFGKVYLA |
| | QDIKTKKRMACKLIPVDQFKPSDVEIQACFRHENIAELYGAVLWGETVHLFMEAGEGG |
| | SVLEKLESCGPMREFEIIWVTKHVLKGLDFLHSKKVIHHDIKPSNIVFMSTKAVLVDF |
| | GLSVQMTEDVYFPKDLRGTEIYMSPEVILCRGHSTKADIYSLGATLIHMQTGTPPWVK |
| | RYPRSAYPSYLYIIHKQAPPLEDIADDCSPGMRELIEASLERNPNHRPRAADLLKHEA |
| | LNPPREDQPRCQSLDSALLERKRLLSRKELELPENIADSSCTGSTEESEMLKRQRSLY |
| | IDLCALAGYFNLVRGPPTLEYG |
| | SEQ ID NO: 21 1770 bp |
| NOV1k, | TTCGTAACAACTCCGCCCCATTGACGCAAATGGGCGGTAGGCGTGTACGGTGGGAGGT |
| 212779063 | |
| DNA Sequence | CTATATAAGCAGAGCTCTCTGGCTAACTAGAGAACCCACTGCTTACTGGCTTATCGAA |
| | ATTAATACGACTCACTATAGGGAGACCCAAGCTGGCTAGCGTTTAAACTTAAGCTTGG |
| | TACCGAGCTCGGATCCACCATGGAGTACATGAGCACTGGAAGTGACAATAAACAAGAG |
| | ATTGATTTATTAATTAAACATTTAAATGTGTCTGATGTAATAGACATTATGGAAAATC |
| | TTTATGCAAGTGAAGAGCCAGCAGTTTATGAACCCAGTCTAATCACCATGTGTCAAGA |
| | CAGTAATCAAAACGATGAGCGTTCTAAGTCTCTGCTGCTTAGTGGCCAAGAGGTACCA |
| | TGGTTGTCATCAGTCAGATATGGAACTGTGGAGGATTTGCTTGCTTTTGCAAACCATA |
| | TATCCAACACTGCAAAGCATTTTTATGGACAACGACCACAGGAATCTGGAATTTTATT |
| | AAACATGGTCATCACTCCCCAAAATGGACGTTACCAAATAGATTCCGATGTTCTCCTG |
| | ATCCCCTGGAAGCTGACTTACAGGAATATTGGTTCTGATTTTATTCCTCGGGGCGCCT |
| | TTGGAAAGGTATACTTGGCACAAGATATAAAGACGAAGAAAAGAATGGCGTGTAAACT |
| | GATCCCAGTAGATCAATTTAAGCCATCTGATGTGGAAATCCAGGCTTGCTTCCGGCAC |
| | GAGAACATCGCAGAGCTGTATGGCGCAGTCCTGTGGGGTGAAACTGTCCATCTCTTTA |
| | TGGAAGCAGGCGAGGGAGGGTCTGTTCTGGAGAAACTGGAGAGCTGTGGACCAATGAG |
| | AGAATTTGAAATTATTTGGGTGACAAAGCATGTTCTCAAGGGACTTGATTTTCTACAC |
| | TCAAAGAAAGTGATCCATCATGATATTAAACCTAGCAACATTGTTTTCATGTCCACAA |
| | AAGCTGTTTTGGTGGATTTTGGCCTAAGTGTTCAAATGACCGAAGATGTCTATTTTCC |
| | TAAGGACCTCCGAGGAACAGAGATTTACATGAGCCCAGAGGTCATCCTGTGCAGGGGC |
| | CATTCAACCAAAGCAGACATCTACAGCCTGGGGGCCACGCTCATCCACATGCAGACGG |
| | GCACCCCACCCTCGGTGAAGCGCTACCCTCGCTCAGCCTATCCCTCCTACCTGTACAT |
| | AATCCACAAGCAAGCACCTCCACTGGAAGACATTGCAGATGACTGCAGTCCAGGGATC |
| | ACAGAGCTGATAGAAGCTTCCCTGGAGAGAAACCCCAATCACCGCCCAAGAGCCGCAG |
| | ACCTACTAAAACATGAGCCCCTGAACCCGCCCAGAGAGGATCAGCCACGCTGTCAGAG |
| | TCTGGACTCTGCCCTCTTGGAGCGCAAGAGGCTGCTGAGTAGGAAGGAGCTGGAACTT |
| | CCTGAGAACATTGCTGATTCTTCGTGCACAGGAAGCACCGAGGAATCTGAGATGCTCA |
| | AGAGGCAACGCTCTCTCTACATCGACCTCGGCGCTCTGGCTGGCTACTTCAATCTTGT |
| | TCGGGGACCACCAACGCTTGAATATGGCTGAGCGGCCGCTCGAGTCTAGAGGGCCCGT |
| | TTAAACCCGCTGATCAGCCTCGACTGTGCCTTCTAGTTGCCAGCCATCTGTTGTTTGC |
| | CCCTCCCCCGTGCCTTCCTTGACCCTGGAAGGTGCCACTCCCACTGTCCTTTCCTAAT |
| | AAAATCAGGAAATTGCATCGCATTGTCTGA |
| | ORF Start: at 137 ORF Stop: TGA at 1595 |
| | SEQ ID NO: 22 486 aa MW at 54926.2kD |
| NOV1k, | GDPSWLAFKLKLGTELCSTMEYMSTGSDNKEEIDLLIKHLNVSDVIDIMENLYASEEP |
| 212779063 | |
| Protein Sequence | AVYEPSLMTMCQDSNQNDERSKSLLLSGQEVPWLSSVRYGTVEDLLAFANHISNTAKH |
| | FYGQRPQESGILLNMVITPQNGRYQIDSDVLLIPWKLTYRNIGSDFIPRGAFGKVYLA |
| | QDIKTKKRMACKLTPVDQFKPSDVEIQACFRHENIAELYGAVLWGETVHLFMEAGEGG |
| | SVLEKLESCGPMREFEIIWVTKHVLKGLDFLHSKKVIHHDIKPSNIVFMSTKAVLVDF |
| | GLSVQMTEDVYFPKDLRGTEIYMSPEVILCRGHSTKADIYSLGATLIHMQTGTPPWVK |
| | RYPRSAYPSYLYIIHKQAPPLEDIADDCSPGMRELIEASLERNPNHRPRAADLLKHEA |
| | LNPPREDQPRCQSLDSALLERKRLLSRKELELPENIADSSCTGSTEESEMLKRQRSLY |
| | IDLGALAGYFNLVRGPPTLEYG |
| | SEQ ID NO: 23 1772 bp |
| NOV11, | TGTCGTAACAACTCCGCCCCATTGACGCAAATGGGCGGTAGGCGTGTACGGTGGGAGG |
| CG101683-02 | |
| DNA Sequence | TCTATATAAGCAGAGCTCTCTGGCTAACTAGAGAACCCACTGCTTACTGGCTTATCGA |
| | AATTAATACGACTCACTATAGGGAGACCCAAGCTGGCTAGCGTTTAAACTTAAGCTTG |
| | GTACCGAGCTCGGATCCACCATGGAGTACATGAGCACTGGAAGTGACAATAAAGAAGA |
| | GATTGATTTATTAATTAAACATTTAAATGTGTCTGATGTAATAGACATTATGGAAAAT |
| | CTTTATGCAAGTGAAGAGCCAGCAGTTTATGAACCCAGTCTAATGACCATGTGTCAAG |
| | ACAGTAATCAAAACGATGAGCGTTCTAAGTCTCTGCTGCTTAGTGGCCAAGAGGTACC |
| | ATGGTTGTCATCAGTCAGATACGGAACTGTGGAGGATTTGCTTGCTTTTGCAAACCAT |
| | ATATCCAACACTGCAAAGCATTTTTATGGACAACGACCACAGGAATCTGGAATTTTAT |
| | TAAACATGGTCATCACTCCCCAAAATGGACGTTACCAAATAGATTCCGATGTTCTCCT |
| | GATCCCCTGGAAGCTGACTTACAGGAATATTGGTTCTGATTTTATTCCTCGGGGCGCC |
| | TTTGGAAAGGTATACTTGGCACAAGATATAAAGACGAAGAAAAGAATGCCGTGTAAAC |
| | TGATCCCAGTAGATCAATTTAAGCCATCTGATGTGGAAATCCAGGCTTGCTTCCGGCA |
| | CGAGAACATCGCAGAGCTGTATGGCGCAGTCCTGTGGGGTGAAACTGTCCATCTCTTT |
| | ATGGAAGCAGGCCAGGGAGGGTCTGTTCTGGAGAAACTGGAGAGCTGTGGACCAATGA |
| | GAGAATTTGAAATTATTTGGGTGACAAAGCATGTTCTCAAGGGACTTGATTTTCTACA |
| | CTCAAAGAAAGTGATCCATCATGATATTAAACCTAGCAACATTGTTTTCATGTCCACA |
| | AAAGCTGTTTTGGTGGATTTTGGCCTAAGTGTTCAAATGACCGAAGATGTCTATTTTC |
| | CTAAGGACCTCCGAGGAACAGAGATTTACATGAGCCCAGAGGTCATCCTGTGCAGGGG |
| | CCATTCAACCAAAGCAGACATCTACAGCCTGGGGGCCACGCTCATCCACATGCAGACG |
| | GGCACCCCACCCTGGGTGAAGCGCTACCCTCGCTCAGCCTATCCCTCCTACCTGTACA |
| | TAATCCACAAGCAAGCACCTCCACTGGAAGACATTGCAGATGACTGCAGTCCAGCGAT |
| | CAGAGAGCTGATAGAAGCTTCCCTGGAGAGAAACCCCAATCACCGCCCAAGAGCCGCA |
| | GACCTACTAAAACATGAGGCCCTGAACCCGCCCAGAGAGGATCAGCCACGCTGTCAGA |
| | GTCTGGACTCTGCCCTCTTGGAGCGCAAGAGGCTGCTGAGTAGGAAGGAGCTGGAACT |
| | TCCTGAGAACATTGCTGATTCTTCGTGCACAGGAAGCACCGAGGAATCTCAGATGCTC |
| | AAGAGGCAACGCTCTCTCTACATCGACCTCGGCGCTCTGGCTGGCTACTTCAATCTTC |
| | TTCGGGGACCACCAACGCTTGAATATGGCTGAGCGGCCGCTCGAGTCTAGAGCGCCCG |
| | TTTAAACCCGCTGATCAGCCTCGACTGTGCCTTCTAGTTGCCAGCCATCTGTTGTTTG |
| | CCCCTCCCCCGTGCCTTCCTTGACCCTGGAAGGTGCCACTCCCACTGTCCTTTCCTAA |
| | TAAAATGAGGAAATTGCATCGCATTGTCTGAG |
| | ORF Start: ATG at 195 ORF Stop: TGA at 1596 |
| | SEQ ID NO: 24 467 aa MW AT 52923.9kD |
| NOV11, | MEYMSTGSDNKEEIDLLIKHLNVSDVIDIMENLYASEEPAVYEPSLMTMCQDSNQNDE |
| CG101683-02 | |
| Protein Sequence | RSKSLLLSGQEVPWLSSVRYGTVEDLLAFANHTSNTAKHFYGQRPQESGILLNMVITP |
| | QNGRYQIDSDVLLIPWKLTYRNIGSDFIPRGAFGKVYLAQDIKTKKRMACKLIPVDQF |
| | KPSDVEIQACFRHENIAELYGAVLWGETVHLFMEAGEGGSVLEKLESCGPMREFEIIW |
| | VTKHVLKGLDFLHSKKVHHDIKPSNIVFMSTKAVLVDFGLSVQMTEDVYFPKDLRGT |
| | EIYMSPEVILCRGHSTKADIYSLGATLIHMQTGTPPWVKRYPRSAYPSYLYITHKQAP |
| | PLEDTADDCSPGMRELIEASLERNPNHRPRAADLLKHEALNPPREDQPRCQSLDSALL |
| | ERKRLLSRKELELPENIADSSCTGSTEESEMLKRQRSLYIDLGALAGYFNLVRGPPTL |
| | SEQ ID NO: 25 1425 bp |
| NOV1m, | ACCATGGAGTACATGAGCACTGGAAGTGACAATAAAGAAGAGATTCATTTATTAATTA |
| CG101683-03 | |
| DNA Sequence | AACATTTAAATGTGTCTGATGTAATAGACATTATGGAAAATCTTTATGCAAGTGAAGA |
| | GCCAGCAGTTTATGAACCCAGTCTAATGACCATGTGTCAAGACAGTAATCAAAACGAT |
| | GAGCGTTCTAAGTCTCTGCTGCTTAGTGGCCAAGAGGTACCATGGTTGTCATCAGTCA |
| | GATACGGAACTGTGGAGGATTTGCTTGCTTTTGCAAACCATATATCCAACACTGCAAA |
| | GCATTTTTATGGACAACGACCACAGGAATCTGGAATTTTATTAAACATGGTCATCACT |
| | CCCCAAAATGGACGTTACCAAATAGATTCCGATGTTCTCCTGATCCCCTGGAAGCTGA |
| | CTTACACGAATATTGGTTCTGATTTTATTCCTCGGGGCGCCTTTGGAAAGGTATACTT |
| | GGCACAAGATATAAAGACGAAGAAAAGAATGGCGTGTAAACTGATCCCAGTAGATCAA |
| | TTTAAGCCATCTGATGTGGAAATCCAGGCTTGCTTCCGGCACGAGAACATCGCAGAGC |
| | TGTATGGCGCAGTCCTGTGGCGTGAAACTGTCCATCTCTTTATGGAAGCAGGCGAGGG |
| | AGGGTCTGTTCTGGAGAAACTGGAGAGCTGTGGACCAATCAGAGAATTTGAAATTATT |
| | TCGGTGACAAAGCATGTTCTCAAGGGACTTGATTTTCTACACTCAAAGAAAGTGATCC |
| | ATCATGATATTAAACCTAGCAACATTGTTTTCATGTCCACAAAAGCTGTTTTGGTGGA |
| | TTTTGGCCTAAGTGTTCAAATGACCGAAGATGTCTATTTTCCTAAGGACCTCCGAGGA |
| | ACAGAGATTTACATGAGCCCAGAGGTCATCCTGTGCAGGGGCCATTCAACCAAAGCAG |
| | ACATCTACAGCCTGGGGGCCACGCTCATCCACATGCAGACGGGCACCCCACCCTGGGT |
| | GAAGCGCTACCCTCCCTCAGCCTATCCCTCCTACCTGTACATAATCCACAAGCAAGCA |
| | CCTCCACTGGAAGACATTGCAGATGACTGCAGTCCAGGGATGAGAGAGCTGATAGAAG |
| | CTTCCCTGGAGAGAAACCCCAATCACCGCCCAAGAGCCGCAGACCTACTAAAACATGA |
| | GGCCCTGAACCCGCCCAGAGAGGATCAGCCACGCTGTCAGAGTCTGGACTCTGCCCTC |
| | TTGGAGCCCAAGAGGCTGCTGAGTAGGAAGGAGCTGGAACTTCCTGAGAACATTGCTG |
| | ATTCTTCGTGCACAGGAAGCACCGAGGAATCTGAGATGCTCAAGAGCCAACGCTCTCT |
| | CTACATCGACCTCGGCGCTCTGGCTGGCTACTTCAATCTTGTTCGGGGACCACCAACG |
| | CTTGAATATGGCCATCATCACCACCATCACTGA |
| | ORF Start: at 1 ORF Stop: TGA at 1423 |
| | SEQ ID NO: 26 474 aa MW at 53847.9kD. |
| NOV1m, | TMEYMSTGSDNKEEIDLLIKHLNVSDVIDIMENLYASEEPAVYEPSLMTMCQDSNQND |
| CG101683-03 | |
| Protein Sequence | ERSKSLLLSGQEVPWLSSVRYGTVEDLLAFANHISNTAKHFYGQRPQESGILLNMVIT |
| | PQNGRYQIDSDVLLIPWKLTYRNIGSDFIPRGAFGKVYLAQDIKTKKRMACKLIPVDQ |
| | FKPSDVEIQACPRHENIAELYGAVLWGETVHLFMEAGEGGSVLEKLESCGPMREFEII |
| | WVTKHVLKGLDFLHSKKVIHHDIKPSNIVFMSTKAVLVDFGLSVQMTEDVYFPKDLRG |
| | TEIYMSPEVILCRGHSTKADIYSLGATLIHMQTGTPPWVKRYPRSAYPSYLYIIHKQA |
| | PPLEDIADDCSPGMRELIEASLERNPNHRPRAADLLKHEALNPPREDQPRCQSLDSAL |
| | LERKRLLSRKELELPENIADSSCTGSTEESEMLKRQRSLYIDLGALAGYFNLVRGPPT |
| | LEYGHHHHHH |
| | SEQ ID NO: 27 1344 bp |
| NOV1n, | ACCATGGAGTACATGAGCACTGGAAGTGACAATAAAGAAGAGATTGATTTATTAATTA |
| CG101683-04 | |
| DNA Sequence | AACATTTAAATGTGTCTGATGTAATAGACATTATGGAAAATCTTTATGCAAGTGAAGA |
| | GCCAGCAGTTTATGAACCCAGTCTAATGACCATGTGTCAAGACAGTAATCAAAACGAT |
| | GAGCGTTCTAAGTCTCTGCTGCTTAGTGGCCAAGAGGTACCATGGTTGTCATCAGTCA |
| | GATACGGAACTGTGGAGGATTTGCTTGCTTTTGCAAACCATATATCCAACACTGCAAA |
| | GCATTTTTATGGACAACGACCACAGGAATCTGGAATTTTATTAAACATGGTCATCACT |
| | CCCCAAAATGGACGTTACCAAATAOATTCCGATGTTCTCCTCATCCCCTGGAAGCTGA |
| | CTTACAGGAATATTGGTTCTGATTTTATTCCTCGGGGCGCCTTTGGAAAGGTATACTT |
| | GGCACAAGATATAAAGACGAAGAAAAGAATGGCGTGTAAACTGATCCCAGTAGATCAA |
| | TTTAAGCCATCTCATGTGGAAATCCAGGCTTGCTTCCGCCACGAGAACATCGCAGAGC |
| | TGTATGCCGCAGTCCTGTGGGGTGAAACTGTCCATCTCTTTATGGAAGCAGGCGAGGG |
| | AGGGTCTGTTCTGGAGAAACTGGAGAGCTGTGGACCAATGAGAGAATTTCAAATTATT |
| | TGGGTGACAAAGCATGTTCTCAAGOGACTTGATTTTCTACACTCAAAGAAAGTGATCC |
| | ATCATGATATTAAACCTAGCAACATTGTTTTCATGTCCACAAAAGCTGTTTTGGTGGA |
| | TTTTCGCCTAAGTGTTCAAATGACCGAAGATGTCTATTTTCCTAAGGACCTCCGAGGA |
| | ACAGAGATTTACATGAGCCCAGAGGTCATCCTGTGCAGGGGCCATTCAACCAAAGCAC |
| | ACATCTACAGCCTGGGGGCCACGCTCATCCACATGCAGACGGGCACCCCACCCTGGGT |
| | GAAGCGCTACCCTCGCTCAGCCTATCCCTCCTACCTGTACATAATCCACAAGCAACCA |
| | CCTCCACTGGAAGACATTGCAGATGACTGCAGTCCAGGGATCAGAGAGCTGATAGAAG |
| | CTTCCCTGGACAGAAACCCCAATCACCGCCCAAGAGCCGCAGACCTACTAAAACATGA |
| | GGCCCTGAACCCGCCCAGAGAGGATCAGCCACGCTGTCAGAGTCTGGACTCTGCCCTC |
| | TTGGAGCGCAAGAGGCTGCTGAGTAGGAAGGAGCTGGAACTTCCTGAGAACATTGCTC |
| | ATCATCACCACCATCACTGAGCGGCCCGCTTCCATCTAGAGCTGCAGTCTCGAGCATG |
| | CGGTACCAGC |
| | ORF Start: at 1 ORF Stop: TGA at 1294 |
| | SEQ ID NO: 28 431 aa MW at 49139.7kD |
| NOV1n, | TMEYMSTGSDNKEEIDLLIKHLNVSDVIDIMENLYASEEPAVYEPSLMTMCQDSNQND |
| CG101683-04 | |
| Protein Sequence | ERSKSLLLSGQEVPWLSSVRYGTVEDLLAFANHISNTAKHFYGQRPQESGILLNMVIT |
| | PQNGRYQIDSDVLLIPWKLTYRNIGSDFIPRGAFGKVYLAQDIKTKKRMACKLIPVDQ |
| | FKPSDVEIQACFRHENIAELYGAVLWGETVHLFMEAGEGGSVLEKLESCGPMREFEII |
| | WVTKHVLKGLDFLHSKKVIHHDIKPSNIVFMSTKAVLVDFGLSVQMTEDVYFPKDLRG |
| | TEIYMSPEVILCRGHSTKADIYSLGATLIHMQTGTPPWVKRYPRSAYPSYLYIIHKQA |
| | PPLEDIADDCSPGMRELIEASLERNPNHRPRAADLLKHEALNPPREDQPRCQSLDSAL |
| | LERKRLLSRKELELPENIAHHHHHH |
| | SEQ ID NO: 29 1327 bp |
| NOV1o, | CCACCATCGGGCGCGGATCCACCATGGGACATCATCACCACCATCACGAGTACATGAG |
| CG101683-05 | |
| DNA Sequence | CACTGGAAGTGACAATAAAGAAGAGATTGATTTATTAATTAAACATTTAAATGTGTCT |
| | GATGTAATAGACATTATGGAAAATCTTTATGCAAGTGAAGAGCCAGCAGTTTATGAAC |
| | CCAGTCTAATGACCATGTGTCAAGACAGTAATCAAAACGATGAGCGTTCTAAGTCTCT |
| | GCTGCTTAGTGGCCAAGAGGTACCATGGTTGTCATCAGTCAGATACGGAACTGTGGAG |
| | GATTTGCTTGCTTTTGCAAACCATATATCCAACACTGCAAAGCATTTTTATGGACAAC |
| | GACCACAGGAATCTGGAATTTTATTAAACATGGTCATCACTCCCCAAAATGGACGTTA |
| | CCAAATAGATTCCGATGTTCTCCTGATCCCCTGGAAGCTGACTTACAGGAATATTGGT |
| | TCTGATTTTATTCCTCGGGGCGCCTTTGGAAAGGTATACTTGGCACAAGATATAAAGA |
| | CGAAGAAAAGAATGGCGTGTAAACTGATCCCAGTAGATCAATTTAAGCCATCTGATGT |
| | GGAAATCCAGGCTTGCTTCCGGCACGAGAACATCGCAGAGCTGTATGGCGCAGTCCTG |
| | TGGGGTGAAACTGTCCATCTCTTTATGGAAGCAGGCGAGGGAGGGTCTGTTCTGGAGA |
| | AACTGGAGAGCTGTGGACCAATGAGAGAATTTGAAATTATTTGGGTGACAAAGCATGT |
| | TCTCAAGGGACTTGATTTTCTACACTCAAACAAAGTGATCCATCATGATATTAAACCT |
| | AGCAACATTGTTTTCATGTCCACAAAAGCTGTTTTGGTGGATTTTGGCCTAAGTGTTC |
| | AAATGACCGAAGATGTCTATTTTCCTAAGGACCTCCGAGGAACAGAGATTTACATGAG |
| | CCCAGAGGTCATCCTGTGCAGGGGCCATTCAACCAAAGCAGACATCTACAGCCTGGGG |
| | GCCACGCTCATCCACATGCAGACGGGCACCCCACCCTGGGTGAAGCGCTACCCTCGCT |
| | CAGCCTATCCCTCCTACCTGTACATAATCCACAAGCAAGCACCTCCACTGGAAGACAT |
| | TGCAGATGACTGCAGTCCAGGGATGAGAGAGCTGATAGAAGCTTCCCTGGAGAGAAAC |
| | CCCAATCACCGCCCAAGAGCCGCAGACCTACTAAAACATGAGGCCCTGAACCCGCCCA |
| | GAGAGGATCAGCCACGCTGTCAGAGTCTGGACTCTGCCCTCTTGGAGCGCAAGAGGCT |
| | GCTGAGTAGGAAGGAGCTGGAACTTCCTGAGAACATTGCTTGAGGCGGCCG |
| | ORF Start at 48 ORF Stop TGA at 1317 |
| | SEQ ID NO: 30 423 aa MW at 48084.5 kD |
| NOV1o, | EYMSTGSDNKEEIDLLIKHLNVSDVIDIMENLYASEEPAVYEPSLMTMCQDSNQNDER |
| CG101683-05 | |
| Protein Sequence | SKSLLLSGQEVPWLSSVRYGTVEDLLAFANHISNTAKHFYGQRPQESGILLNMVITPQ |
| | NGRYQIDSDVLLIPWKLTYRNIGSDFIPRGAFGKVYLAQDIKTKKRMACKLIPVDQFK |
| | PSDVEIQACFRHENIAELYGAVLWGETVHLFMEAGEGGSVLEKLESCGPMREFEIIWV |
| | TKHVLKGLDFLHSKKVIHHDIKPSNTVFMSTKAVLVDFGLSVQMTEDVYFPKDLRGTE |
| | IYMSPEVILCRGHSTKADIYSLGATLIHMQTGTPPWVKRYPRSAYPSYLYIIHKQAPP |
| | LEDIADDCSPGMRELIEASLERNPNHRPRAADLLKHEALNPPREDQPRCQSLDSALLE |
| | RKRLLSRKELELPENIA |
| | SEQ ID NO: 31 1428 bp |
| NOV1p, | ACCATGGGACATCATCACCACCATCACGAGTACATGAGCACTGGAAGTGACAATAAAG |
| CG1 01683-06 | |
| DNA Sequence | AAGAGATTGATTTATTAATTAAACATTTAAATGTGTCTGATGTAATAGACATTATGGA |
| | AAATCTTTATGCAAGTGAAGAGCCAGCAGTTTATGAACCCAGTCTAATGACCATGTGT |
| | CAAGACAGTAATCAAAACGATGAGCGTTCTAAGTCTCTGCTGCTTAGTGGCCAAGAGG |
| | TACCATGGTTGTCATCAGTCAGATACGGAACTGTGCAGGATTTGCTTGCTTTTGCAAA |
| | CCATATATCCAACACTGCAAAGCATTTTTATGGACAACGACCACAGGAATCTGGAATT |
| | TTATTAAACATGGTCATCACTCCCCAAAATGGACGTTACCAAATAGATTCCGATGTTC |
| | TCCTGATCCCCTGGAAGCTGACTTACAGGAATATTGGTTCTGATTTTATTCCTCGGGG |
| | CGCCTTTGGAAAGGTATACTTGGCACAAGATATAAAGACGAAGAAAAGAATGGCGTGT |
| | AAACTGATCCCAGTAGATCAATTTAAGCCATCTGATGTGGAAATCCAGGCTTGCTTCC |
| | GGCACGAGAACATCGCAGAGCTGTATGGCGCAGTCCTGTGGGGTGAAACTGTCCATCT |
| | CTTTATGGAAGCAGGCGAGGGAGGGTCTGTTCTGGAGAAACTGGAGAGCTGTGGACCA |
| | ATGAGAGAATTTGAAATTATTTGGGTGACAAAGCATGTTCTCAAGGGACTTGATTTTC |
| | TACACTCAAAGAAAGTGATCCATCATGATATTAAACCTAGCAACATTGTTTTCATGTC |
| | CACAAAAGCTGTTTTGGTGGATTTTGGCCTAAGTGTTCAAATGACCGAAGATGTCTAT |
| | TTTCCTAAGGACCTCCCAGGAACAGAGATTTACATGAGCCCAGAGGTCATCCTGTCCA |
| | GGGGCCATTCAACCAAAGCAGACATCTACAGCCTGGGGGCCACGCTCATCCACATGCA |
| | GACGGGCACCCCACCCTGGGTGAAGCGCTACCCTCGCTCAGCCTATCCCTCCTACCTG |
| | TACATAATCCACAAGCAAGCACCTCCACTGGAAGACATTGCAGATGACTGCAGTCCAG |
| | GGATGAGAGAGCTGATAGAAGCTTCCCTGGAGAGAAACCCCAATCACCGCCCAAGAGC |
| | CGCAGACCTACTAAAACATGAGGCCCTGAACCCGCCCAGAGAGGATCAGCCACGCTGT |
| | CAGAGTCTGGACTCTGCCCTCTTCGAGCGCAAGAGGCTGCTGAGTAGGAAGGAGCTGG |
| | AACTTCCTGAGAACATTGCTGATTCTTCGTGCACAGGAAGCACCGACGAATCTGAGAT |
| | GCTCAAGAGGCAACGCTCTCTCTACATCGACCTCGGCGCTCTGGCTGGCTACTTCAAT |
| | CTTGTTCGGGGACCACCAACGCTTGAATATGGCTGA |
| | ORF Start: at 1 ORF Stop: TGA at 1426 |
| | SEQ ID NO: 32 475 aa MW at 53904.9kD |
| NOV1p, | TMGHHHHHHEYMSTGSDNKEEIDLLIKHLNVSDVIDIMENLYASEEPAVYEPSLMTMC |
| CG101683-06 | |
| Protein Sequence | QDSNQNDERSKSLLLSGQEVPWLSSVRYGTVEDLLAFANHISNTAKHFYGQRPQESGI |
| | LLNMVITPQNGRYQIDSDVLLIPWKLTYRNIGSDFIPRGAFGKVYLAQDIKTKKRMAC |
| | KLIPVDQFKPSDVEIQACFRHENIAELYGAVLWGETVHLFMEAGEGGSVLEKLESCGP |
| | MREFEIIWVTKHVLKGLDFLHSKKVIHHDIKPSNIVFMSTKAVLVDFGLSVQMTEDVY |
| | FPKDLRGTEIYMSPEVILCRGHSTKADIYSLGATLIHMQTGTPPWVKRYPRSAYPSYL |
| | YIIHKQAPPLEDIADDCSPGMRELIEASLERNPNHRPRAADLLKHEALNPPREDQPRC |
| | QSLDSALLERKRLLSRKELELPENIADSSCTGSTEESEMLKRQRSLYIDLGALAGYFN |
| | LVRGPPTLEYG |
| | SEQ ID NO: 33 1293 bp |
| NOV1q, | CGGCCCCTGGGATCCACCATGGAGTACATGAGCACTGGAAGTCACAATAAAGAAGAGA |
| CG1O1683-07 | |
| DNA Sequence | TTGATTTATTAATTAAACATTTAAATGTGTCTGATGTAATAGACATTATGGAAAATCT |
| | TTATGCAAGTGAAGAGCCAGCAGTTTATGAACCCAGTCTAATGACCATGTGTCAAGAC |
| | AGTAATCAAAACGATGAGCGTTCTAAGTCTCTGCTGCTTAGTGGCCAAGAGGTACCAT |
| | GGTTGTCATCAGTCAGATACGGAACTGTGGAGGATTTGCTTGCTTTTGCAAACCATAT |
| | ATCCAACACTGCAAAGCATTTTTATGGACAACGACCACAGGAATCTGGAATTTTATTA |
| | AACATGGTCATCACTCCCCAAAATGGACGTTACCAAATAGATTCCCATGTTCTCCTGA |
| | TCCCCTGGAAGCTGACTTACAGGAATATTGGTTCTGATTTTATTCCTCGGGGCGCCTT |
| | TGGAAAGGTATACTTGGCACAAGATATAAAGACGAAGAAAAGAATGGCGTGTAAACTG |
| | ATCCCAGTAGATCAATTTAAGCCATCTGATGTGGAAATCCAGGCTTGCTTCCGGCACG |
| | AGAACATCGCAGAGCTGTATGGCGCAGTCCTGTGGGGTGAAACTGTCCATCTCTTTAT |
| | GGAAGCAGGCGAGGGAGGGTCTGTTCTGGAGAAACTGGAGAGCTGTGGACCAATGAGA |
| | GAATTTGAAATTATTTGGGTGACAAAGCATGTTCTCAAGGGACTTGATTTTCTACACT |
| | CAAAGAAAGTGATCCATCATGATATTAAACCTAGCAACATTGTTTTCATGTCCACAAA |
| | AGCTGTTTTGGTGGATTTTGGCCTAAGTGTTCAAATGACCGAAGATGTCTATTTTCCT |
| | AAGGACCTCCGAGGAACAGAGATTTACATGACCCCAGAGGTCATCCTGTGCAGGGGCC |
| | ATTCAACCAAAGCAGACATCTACAGCCTGGGGGCCACGCTCATCCACATGCAGACGGG |
| | CACCCCACCCTGGGTGAAGCGCTACCCTCGCTCAGCCTATCCCTCCTACCTGTACATA |
| | ATCCACAAGCAAGCACCTCCACTGGAAGACATTGCAGATGACTGCAGTCCAGGGATGA |
| | GAGAGCTGATAGAAGCTTCCCTGCACAGAAACCCCAATCACCGCCCAAGAGCCGCACA |
| | CCTACTAAAACATGAGGCCCTGAACCCGCCCAGAGAGGATCAGCCACGCTGTCAGAGT |
| | CTCGACTCTGCCCTCTTGGAGCGCAAGAGGCTGCTGAGTAGGAAGGAGCTGGAACTTC |
| | CTGAGAACATTGCTTGA |
| | ORF Start: ATG at 19 ORF Stop: TGA at 1291 |
| | SEQ ID NO: 34 424 aa MW at 48215.7 kD |
| NOV1q, | MEYMSTGSDNKEEIDLLIKHLNVSDVIDIMENLYASEEPAVYEPSLMTMCQDSNQNDE |
| CG101683-07 | |
| Protein Sequence | RSKSLLLSGQEVPWLSSVRYGTVEDLLAFANHISNTAKHFYGQRPQESGILLNMVITP |
| | QNGRYQIDSDVLLIPWKLTYRNIGSDFIPRGAFGKVYLAQDIKTKKRMACKLIPVDQF |
| | KPSDVEIQACFRHENIAELYGAVLWGETVHLFMEACEGGSVLEKLESCCPMREFEIIW |
| | VTKHVLKGLDFLHSKKVIHHDIKPSNIVFMSTKAVLVDFGLSVQMTEDVYFPKDLRGT |
| | EIYMSPEVILCRGHSTKADIYSLGATLIHMQTGTPPWVKRYPRSAYPSYLYIIHKQAP |
| | PLEDIADDCSPGMRELIEASLERNPNHRPRAADLLKHEALNPPREDQPRCQSLDSALL |
| | ERKRLLSRKELELPENIA |
| | SEQ ID NO: 35 1428 bp |
| NOV1R, | CACCGCGGCCGCACCATGGAGTACATGAGCACTGGAAGTGACAATAAAGAAGAGATTG |
| CG101683-08 | |
| DNA Sequence | ATTTATTAATTAAACATTTAAATGTGTCTGATGTAATAGACATTATGGAAAATCTTTA |
| | TGCAAGTGAAGAGCCAGCAGTTTATGAACCCAGTCTAATGACCATGTGTCAAGACAGT |
| | AATCAAAACGATGAGCGTTCTAAGTCTCTGCTGCTTAGTGGCCAAGAGGTACCATGGT |
| | TGTCATCAGTCAGATACGGAACTGTGGAGGATTTCCTTGCTTTTGCAAACCATATATC |
| | CAACACTGCAAAGCATTTTTATGGACAACGACCACAGGAATCTGGAATTTTATTAAAC |
| | ATGGTCATCACTCCCCAAAATGGACGTTACCAAATAGATTCCGATGTTCTCCTGATCC |
| | CCTGGAAGCTGACTTACAGGAATATTGGTTCTGATTTTATTCCTCGGGGCGCCTTTGG |
| | AAAGGTATACTTGGCACAAGATATAAAGACGAAGAAAAGAATGGCGTGTAAACTGATC |
| | CCAGTAGATCAATTTAAGCCATCTGATGTGGAAATCCAGGCTTGCTTCCGGCACGAGA |
| | ACATCGCAGAGCTGTATGGCGCAGTCCTGTGGGGTGAAACTGTCCATCTCTTTATGGA |
| | AGCAGGCGAGGGAGGGTCTGTTCTGGAGAAACTGGAGAGCTGTGGACCAATGAGAGAA |
| | TTTGAAATTATTTGGGTGACAAAGCATGTTCTCAAGGGACTTGATTTTCTACACTCAA |
| | AGAAAGTGATCCATCATGATATTAAACCTAGCAACATTGTTTTCATGTCCACAAAAGC |
| | TGTTTTGGTGGATTTTGGCCTAAGTGTTCAAATGACCGAAGATGTCTATTTTCCTAAG |
| | GACCTCCGAGGAACAGAGATTTACATGAGCCCAGAGGTCATCCTGTGCAGGGGCCATT |
| | CAACCAAAGCAGACATCTACAGCCTGGGGGCCACGCTCATCCACATGCAGACGGGCAC |
| | CCCACCCTGGGTGAAGCGCTACCCTCGCTCAGCCTATCCCTCCTACCTGTACATAATC |
| | CACAAGCAAGCACCTCCACTGGAAGACATTGCAGATGACTGCAGTCCAGGGATGAGAG |
| | AGCTGATAGAAGCTTCCCTGGAGAGAAACCCCAATCACCGCCCAAGAGCCGCAGACCT |
| | ACTAAAACATGAGGCCCTGAACCCGCCCAGAGAGGATCAGCCACGCTGTCAGAGTCTG |
| | GACTCTGCCCTCTTGGAGCGCAAGAGGCTGCTGAGTAGGAAGGAGCTGGAACTTCCTG |
| | AGAACATTGCTGATTCTTCGTGCACAGGAAGCACCCAGGAATCTGAGATGCTCAAGAG |
| | GCAACGCTCTCTCTACATCGACCTCGGCGCTCTGGCTGGCTACTTCAATCTTGTTCGG |
| | GGACCACCAACGCTTGAATATGGCTAGGTCGACGGC |
| | ORF Start: ATG at 16 ORF Stop: TAG at 1417 |
| | SEQ ID NO: 36 467 aa MW at 52923.9 kD |
| NOV1r, | MEYMSTGSDNKEEIDLLIKHLNVSDVIDIMENLYASEEPAVYEPSLMTMCQDSNQNDE |
| CG101683-08 | |
| Protein Sequence | RSKSLLLSGQEVPWLSSVRYGTVEDLLAFANHISNTAKHFYGQRPQESGILLNMVITP |
| | QNGRYQIDSDVLLIPWKLTYRNIGSDFIPRGAFGKVYLAQDIKTKKRMACKLIPVDQF |
| | KPSDVEIQACFRHENIAELYGAVLWGETVHLFMEAGEGGSVLEKLESCGPMREFEIIW |
| | VTKHVLKGLDFLHSKKVIHHDIKPSNIVFMSTKAVLVDFGLSVQMTEDVYFPKDLRGT |
| | EIYMSPEVILCRCHSTKADIYSLGATLIHMQTGTPPWVKRYPRSAYPSYLYIIHKQAP |
| | PLEDIADUCSPGMRELIEASLERNPNHRPRAADLLKHEALNPPREDQPRCQSLDSALL |
| | ERKRLLSRKELELPENIADSSCTGSTEESEMLKRQRSLYIDLGALAGYFNLVRGPPTL |
| | EYG |

Sequence comparison of the above protein sequences yields the following sequence relationships shown in Table 1B.

TABLE 1B
Comparison of NOV1a against NOV1b through NOV1r.

| | | Identities/ |
|---|---|---|
| | | Similarities for |
| Protein | NOV1a Residues/ | the Matched |
| Sequence | Match Residues | Region |
| NOV1b | 1 . . . 467 | 466/467 (99%) |
| | 2 . . . 468 | 466/467 (99%) |
| NOV1c | 1 . . . 424 | 423/424 (99%) |
| | 5 . . . 428 | 423/424 (99%) |
| NOV1d | 1 . . . 467 | 466/467 (99%) |
| | 2 . . . 468 | 466/467 (99%) |
| NOV1e | 2 . . . 467 | 465/466 (99%) |
| | 13 . . . 478 | 465/466 (99%) |
| NOV1f | 1 . . . 424 | 423/424 (99%) |
| | 2 . . . 425 | 423/424 (99%) |
| NOV1g | 2 . . . 424 | 422/423 (99%) |
| | 16 . . . 438 | 422/423 (99%) |
| NOV1h | 2 . . . 467 | 465/466 (99%) |
| | 10 . . . 475 | 465/466 (99%) |
| NOV1i | 1 . . . 467 | 466/467 (99%) |
| | 5 . . . 471 | 466/467 (99%) |
| NOV1j | 1 . . . 467 | 466/467 (99%) |
| | 20 . . . 486 | 466/467 (99%) |
| NOV1k | 1 . . . 467 | 466/467 (99%) |
| | 20 . . . 486 | 466/467 (99%) |
| NOV1l | 1 . . . 467 | 466/467 (99%) |
| | 1 . . . 467 | 466/467 (99%) |
| NOV1m | 1 . . . 467 | 466/467 (99%) |
| | 2 . . . 468 | 466/467 (99%) |
| NOV1n | 1 . . . 424 | 423/424 (99%) |
| | 2 . . . 425 | 423/424 (99%) |
| NOV1o | 2 . . . 424 | 422/423 (99%) |
| | 1 . . . 423 | 422/423 (99%) |
| NOV1p | 2 . . . 467 | 465/466 (99%) |
| | 10 . . . 475 | 465/466 (99%) |
| NOV1q | 1 . . . 424 | 423/424 (99%) |
| | 1 . . . 424 | 423/424 (99%) |
| NOV1r | 1 . . . 467 | 466/467 (99%) |
| | 1 . . . 467 | 466/467 (99%) |

Further analysis of the NOV1a protein yielded the following properties shown in Table 1C.

TABLE 1C
Protein Sequence Properties NOV1a

| | |
|---|---|
| PSort | 0.6500 probability located in cytoplasm; |
| analysis: | 0.1000 probability located in mitochondrial |
| | matrix space; 0.1000 probability located in |
| | lysosome (lumen); 0.0000 probability |
| | located in endoplasmic reticulum (membrane) |
| SignalP | No Known Signal Sequence Predicted |
| analysis: | |

A search of the NOV1a protein against the Geneseq database, a proprietary database that contains sequences published in patents and patent publication, yielded several homologous proteins shown in Table 1D.

TABLE 1D
Geneseq Results for NOV1a

| | | | Identities/ | |
|---|---|---|---|---|
| | | | Similarities for | |
| Geneseq | Protein/Organism/Length | NOV1a Residues/ | the Matched | Expect |
| Identifier | [Patent #, Date] | Match Residues | Region | Value |
| AAE05951 | Human cot oncoprotein encoded by | 1 . . . 467 | 467/467 (100%) | 0.0 |
| | D14497 oncogene - Homo sapiens, | 1 . . . 467 | 467/467 (100%) | |
| | 467 aa. [US6265216-B1, 24 JUL. | | | |
| | 2001] | | | |
| AAY79244 | Human COT - Homo sapiens, 467 | 1 . . . 467 | 467/467(100%) | 0.0 |
| | aa. [WO200011191-A2, 02 MAR. | 1 . . . 467 | 467/467(100%) | |
| | 2000] | | | |
| AAE10313 | Human Tp12 protein - Homo | 1 . . . 467 | 466/467 (99%) | 0.0 |
| | sapiens, 467 aa. [WO200166559- | 1 . . . 467 | 466/467 (99%) | |
| | A1, 13 SEP. 2001] | | | |
| AAE10314 | Rat Tp12 protein - Rattus sp. 467 | 1 . . . 467 | 439/467 (94%) | 0.0 |
| | aa. [WO200166559-A1, 13 SEP. | 1 . . . 467 | 454/467 (97%) | |
| | 2001] | | | |
| AAY79243 | Rat TPL-2 - Rattus norvegicus, 467 | 1 . . . 467 | 438/467 (93%) | 0.0 |
| | aa. [WO200011191-A2, 02 MAR. | 1 . . . 467 | 453/467 (96%) | |
| | 2000] | | | |

In a BLAST search of public sequence datbases, the NOV1a protein was found to have homology to the proteins shown in the BLASTP data in Table 1E.

TABLE 1E
Public BLASTP Results for NOV1a

| | | | Identities/ | |
|---|---|---|---|---|
| Protein | | | Similarities for | |
| Accession | | NOV1a Residues/ | the Matched | Expect |
| Number | Protein/Organism/Length | Match Residues | Portion | Value |
| P41279 | Mitogen-activated protein kinase | 1 . . . 467 | 467/467 (100%) | 0.0 |
| | kinase kinase 8 (EC 2.7.1.-) (COT | 1 . . . 467 | 467/467 (100%) | |
| | proto-oncogene serine/threonine- | | | |
| | protein kinase) (C-COT) (Cancer | | | |
| | Osaka thyroid oncogene) - Homo | | | |
| | sapiens (Human), 467 aa. | | | |
| A48713 | serine/threonine-specific protein | 1 . . . 467 | 466/467 (99%) | 0.0 |
| | kinase cot, 58 K form - human, 467 | 1 . . . 467 | 466/467 (99%) | |
| | aa. | | | |
| Q63562 | Mitogen-activated protein kinase | 1 . . . 467 | 438/467 (93%) | 0.0 |
| | kinase kinase 8 (EC 2.7.1.-) (Tumor | 1 . . . 467 | 453/467 (96%) | |
| | progression locus 2) (TPL-2) - Rattus | | | |
| | norvegicus (Rat), 467 aa. | | | |
| Q07174 | Mitogen-activated protein kinase | 1 . . . 467 | 435/467 (93%) | 0.0 |
| | kinase kinase 8 (EC 2.7.1.-) (COT | 1 . . . 467 | 454/467 (97%) | |
| | proto-oncogene serine/threonine- | | | |
| | protein kinase) (C-COT) (Cancer | | | |
| | Osaka thyroid oncogene) - Mus | | | |
| | musculus (Mouse), 467 aa. | | | |
| A41253 | kinase-related transforming protein | 1 . . . 397 | 379/397 (95%) | 0.0 |
| | (EC 2.7.1.-) - human, 415 aa. | 1 . . . 397 | 379/397 (95%) | |

PFam analysis predicts that the NOV1a protein contains the domains shown in the Table 1F.

TABLE 1F
Domain Analysis of NOV1a

| | | Identities/ | |
|---|---|---|---|
| | | Similarities for | |
| Pfam | NOV1a | the Matched | Expect |
| Domain | Match Region | Region | Value |
| pkinase | 146 . . . 388 | 74/279 (27%) | 4.7e−54 |
| | | 187/279 (67%) | |

### Example 2

The NOV2 clone was analyzed, and the nucleotide and encoded polypeptide sequences are shown in Table 2A.

TABLE 2A
NOV2 Sequence Analysis

| | |
|---|---|
| | SEQ ID NO: 37 917 bp |
| NOV2a, | GATGAAGAGAGGGGAGCTCTTTGACTACCTCACTGAGAAGGTCACCTTGAGTGAGAAG |
| CG101996-01 | |
| DNA Sequence | GAAACCAGAAACATCATGCGAGCTCTGCTGGAGGTGATCTGCACCTTGCACAAACTCA |
| | ACATCGTGCACCCGGACCTGAAGCCCGAGAACATTCTCTTGGATGACAACATGAACAT |
| | CAAGCTCACAGACTTTGGCTTTTCCTGCCAGCTGGAGCCGGGACAGAGGCTGCGAGGG |
| | TCTGCGGGACCCCCAGTTACCTGGCCCCTGAGATTATCGAGTGCTCCATGAATGACGA |
| | CCACCCGGGCTACGGGAAAGAGGTGGACATGTGGAGCACTGGCGTCATCATGTACACG |
| | CTGCTGGCCGCTCCCCGCCCTTCTGGCACCGGAAGCAGATGCTGATGCTGAGGATGAT |
| | CATGAGCGGCAACTACCAGTTTGGCTCGCCCGAGTGGGATGATTACTCGGACACCGTG |
| | AAGGACCTGGTCTCCCGATTCCTGGTGGTGCAACCCCAGAACCCCTACACAGCGCAAG |
| | AGGCCTTGGCACACCCCTTCTTCCAGCAGTACTTGGTGGAGGAAGTGCGGCACTTCAG |
| | CCCCCGGGGGAAGTTCAAGGTGATCGCTCTGACCGTGCTGGCTTCAGTGCGGATCTAC |
| | TACCAGTACCGCCGGGTGAAGCCTGTGACCCGGGAGATCGTCATCCGAGACCCCTATG |
| | CCCTCCGGCCTCTGCGCCGGCTCATCGACGCCTACGCTTTCCGAATCTATGGCCACTG |
| | GGTGAAGAAGGGGCAGCAGCAGAACCGGGCAGCCCTTTTCCAGAACACACCCAAGGCC |
| | GTGCTCCTCTCCCTGCCCGAGGAGGACTACTGAGGGGCTGGCCAGTCAGGGAGGGCTA |
| | GGGGGCAGGTGGGGAGGGGAAGCCATGGAAATACAAGTCAAAGGGGT |
| | ORF Start: ATG at 387 ORF Stop: TGA at 843 |
| | SEQ ID NO: 38 152 aa MW at 18023.7 kD |
| NOV2a, | MLMLRMIMSGNYQFGSPEWDDYSDTVKDLVSRFLVVQPQNRYTAEEALAHPFFQQYLV |
| CG101996-01 | |
| Protein Sequence | EEVRHFSPRGKFKVIALTVLASVRIYYQYRRVKPVTREIVIRDPYALRPLRRLIDAYA |
| | FRIYGNWVKKGQQQNRAALFENTPKAVLLSLAEEDY |
| | SEQ ID NO: 39 1299 bp |
| NOV2b, | ATGACCCGGGACGAGGCACTGCCGGACTCTCATTCTGCACAGGACTTCTATGAGAATT |
| CG101996-04 | |
| DNA Sequence | ATGAGCCCAAAGAGATCCTGGGCAGCGGCGTTAGCAGTGTGGTCAGGCGATGCATCCA |
| | CAAGCCCACGAGCCAGGAGTACGCCGTGAAGGTCATCGACGTCACCGGTGGAGGCAGC |
| | TTCAGCCCGGAGGAGGTGCGGGAGCTGCGAGAAGCCACGCTGAAGGAGGTGGACATCC |
| | TGCGCAAGGTCTCAGGGCACCCCAACATCATACAGCTGAAGGACACTTATGAGACCAA |
| | CACTTTCTTCTTCTTGGTGTTTGACCTGATGAAGAGAGGGGAGCTCTTTGACTACCTC |
| | ACTGAGAAGGTCACCTTGAGTGAGAAGGAAACCAGAAAGATCATGCGAGCTCTGCTGG |
| | AGGTGATCTGCACCTTGCACAAACTCAACATCGTGCACCGGGACCTGAAGCCCGAGAA |
| | CATTCTCTTGGATGACAACATGAACATCAACCTCACAGACTTTGGCTTTTCCTGCCAG |
| | CTGGAGCCGGGAGAGAGGCTGCGAGTAGAGACAGGGTTTCACCATGTTGGTCAGGCTG |
| | GTCTCGAACTCCTGACCTTACGATCCGCCCGCCTCGGCCTCCCAAAGTGCTGTGATTA |
| | CAGGCGTGAGCCACCATGCCCAGCAGGGCTAGGCATTTCTTCAGAGGTCTGCGGGACC |
| | CCCAGTTACCTGCCCCCTGAGATTATCGAGTGCTCCATGAATGAGGACCACCCGGGCT |
| | ACGGGAAAGAGGTGGACATGTGGAGCACTGGCGTCATCATGTACACGCTGCTGGCCGG |
| | CTCCCCGCCCTTCTGGCACCGGAAGCAGATGCTGATGCTGAGGATGATCATGAGCGGC |
| | AACTACCAGTTTGGCTCGCCCGAGTGGGATGATTACTCGGACACCGTGAAGGACCTGG |
| | TCTCCCGATTCCTGGTGGTCCAACCCCAGAACCGCTATACAGCGGAAGAGGCCTTGGC |
| | ACACCCCTTCTTCCAGCAGTACTTGGTAGAGGAAGTGCGGCACTTCAGCCCCCGGGGG |
| | AAGTTCAAGGTGATCGCTCTGACCGTGCTGGCTTCAGTGCGGATCTACTACCACTACC |
| | GCCGGGTGAAGCCTGTGACCCGGGAGATCGTCATCCGAGACCCCTATGCCCTCCGGCC |
| | TCTGCGCCGGCTCATCGACGCCTACGCTTTCCGAATCTATGGCCACTGGGTGAAGAAC |
| | GGGCAGCAGCAGAACCGGGCAGCCCTTTTCGAGAACACACCCAAGGCCGTGCTCCTCT |
| | CCCTGGCCGAGGAGGACTACTGA |
| | ORF Start: ATG at 1 ORF Stop: TGA at 1297 |
| | SEQ ID NO: 40 432 aa MW at 49811.7 kD |
| NOV2b, | MTRDEALPDSHSAQDFYENYEPKEILGRGVSSVVRRCIHKPTSQEYAVKVIDVTGGGS |
| CG101996-04 | |
| Protein Sequence | FSPEEVRELREATLKEVDILRKVSGHPNIIQLKDTYETNTFFFLVFDLMKRGELFDYL |
| | TEKVTLSEKETRKIMRALLEVICTLHKLNIVHRDLKPENILLDDNMNIKLTDFGFSCQ |
| | LEPGERLRVETGFHHVGQAGLELLTLRSARLGLPKCCDYRREPPCPAGLGISSEVCGT |
| | PSYLAPETIECSMNEDHPGYGKEVDMWSTGVIMYTLLAGSPPFWHRKQMLMLRMIMSG |
| | NYQFGSPEWDDYSDTVKDLVSRFLVVQPQNRYTAEEALAHPFFQQYLVEEVRHFSPRG |
| | KFKVIALTVLASVRIYYQYRRVKPVTREIVIRDPYALRPLRRLIDAYAFRIYGHWVKK |
| | GQQQNRAALFENTPKAVLLSLAEEDY |
| | SEQ ID NO: 41 1377 bp |
| NOV2c, | GGCCTTCAGCCCTCTGTGGTCCCCTCTCCCCGGGGGGCTTTGGGATTCTTGTCAAGCT |
| CG101996-02 | |
| DNA Sequence | CCTTCAAGAGCCTGCAAGCACTTAACCAGCCACCCAGAGTTCCCTCACTGAAGATCTG |
| | AGCATGACCCGGGACCAGGCACTGCCGGACTCTCATTCTGCACAGGACTTCTATGAGA |
| | ATTATGAGCCCAAAGAGATCCTGGGCAGGGGCGTTAGCAGTGTGGTCAGGCGATGCAT |
| | CCACAACCCCACGAGCCAGGAGTACGCCGTGAAGGTCATCGACGTCACCGGTGGAGGC |
| | AGCTTCAGCCCGGAGGAGGTGCGGGAGCTGCGAGAAGCCACGCTGAAGGAGGTGGACA |
| | TCCTGCGCAAGGTCTCAGGGCACCCCAACATCATACAGCTGAAGGACACTTATGAGAC |
| | CAACACTTTCTTCTTCTTGGTGTTTGACCTGATGAAGAGAGCGGAGCTCTTTGACTAC |
| | CTCACTGAGAAGGTCACCTTGAGTGAGAAGGAAACCAGAAAGATCATGCGAGCTCTGC |
| | TGGAGGTGATCTGCACCTTGCACAAACTCAACATCGTGCACCGGGACCTGAAGCCCGA |
| | GAACATTCTCTTGGATGACAACATGAACATCAAGCTCACAGACTTTGGCTTTTCCTGC |
| | CAGCTGGAGCCGGGAGAGAGGCTGCGAGAGGTCTGCGGGACCCCCAGTTACCTGCCCC |
| | CTGAGATTATCGAGTGCTCCATGAATGAGGACCACCCGGGCTACGGGAAAGAGGTGGA |
| | CATGTGGAGCACTCGCGTCATCATGTACACGCTGCTGGCCGGCTCCCCGCCCTTCTGG |
| | CACCGGAAGCAGATGCTGATGCTGAGGATGATCATGAGCGGCAACTACCAGTTTGGCT |
| | CGCCCGAGTGGGATGATTACTCGGACACCGTGAAGGACCTGGTCTCCCGATTCCTGGT |
| | GGTGCAACCCCAGAACCGCTACACAGCGGAAGAGGCCTTGGCACACCCCTTCTTCCAC |
| | CAGTACTTGGTGGAGGAAGTGCGGCACTTCAGCCCCCGGGGGAAGTTCAAGGTGATCG |
| | CTCTGACCGTCCTGGCTTCAGTGCGGATCTACTACCAGTACCGCCGGGTGAAGCCTGT |
| | GACCCGGGAGATCGTCATCCGAGACCCCTATGCCCTCCGGCCTCTGCGCCGGCTCATC |
| | GACGCCTACGCTTTCCGAATCTATGGCCACTGGGTGAAGAAGGGGCAGCAGCAGAACC |
| | GGGCAGCCCTTTTCGAGAACACACCCAAGGCCGTGCTCCTCTCCCTGGCCGAGGAGGA |
| | CTACTGAGGGGCTGGCCAGTCAGGGAGGGCTAGGGGGCAGGTGGGGAGGGGAAGCCAT |
| | GCAAATACAAGTCAAAGGGGTAAAAAAAAAAAAAAAAAAAAAA |
| | ORF Start:ATG at 120 ORF Stop: TGA at 1281 |
| | SEQ ID NO: 42 387 aa MW at 45023.3 kD |
| NOV2c, | MTRDEALPDSHSAQDFYENYEPKEILGRGVSSVVRRCIHKPTSQEYAVKVIDVTGGGS |
| CG101996-02 | |
| Protein Sequence | FSPEEVRELREATLKEVDILRKVSGHPNIIQLKDTYETNTFFFLVFDLMKRGELFDYL |
| | TEKVTLSEKETRKIMRALLEVICTLHKLNIVHRDLKPENILLDDNMNIKLTDFGFSCQ |
| | LEPGERLREVCGTPSYLAPEIIECSMNEDHPGYGKEVDMWSTGVIMYTLLAGSPPFWH |
| | RKQMLMLRMIMSGNYQFGSPEWDDYSDTVKDLVSRFLVVQPQNRYTAEEALAHPFFQQ |
| | YLVEEVRHFSPRGKFKVIALTVLASVRIYYQYRRVKPVTREIVIRDPYALRPLRRLID |
| | AYAFRIYGHWVKKGQQQNRAALFENTPKAVLLSLAEEDY |
| | SEQ ID NO: 43 1165 bp |
| NOV2d, | CATGACCCGGGACGAGGCACTGCCGGACTCTCATTCTGCACACGACTTCTATGAGAAT |
| 245245680 | |
| DNA Sequence | TATGAGCCCAAAGAGATCCTGGGCAGGGGCGTTAGCAGTGTGGTCAGGCGATGCATCC |
| | ACAAGCCCACGAGCCAGGAGTACGCCGTGAAGGTCATCGACGTCACCGGTGGAGGCAG |
| | CTTCAGCCCGGAGGAGGTGCGGGAGCTGCGAGAAGCCACGCTGAAGGAGGTGGACATC |
| | CTGCGCAAGGTCTCAGGGCACCCCAACATCATACAGCTGAAGGACACTTATGAGACCA |
| | ACACTTTCTTCTTCTTGGTGTTTGACCTGATGAAGAGAGGGGAGCTCTTTGACTACCT |
| | CACTGAGAAGGTCACCTTGAGTGAGAAGGAAACCAGAAAGATCATGCGAGCTCTGCTG |
| | GAGGTGATCTGCACCTTGCACAAACTCAACATCGTGCACCGGGACCTGAAGCCCGAGA |
| | ACATTCTCTTGGATGACAACATGAACATCAAGCTCACAGACTTTGGCTTTTCCTGCCA |
| | GCTGGAGCCGGGAGAGAGGCTGCGAGAGGTCTGCGGGACCCCCAGTTACCTGGCCCCT |
| | GAGATTATCGAGTGCTCCATGAATGAGGACCACCCGGGCTACGGGAAAGAGGTGGACA |
| | TGTGGAGCACTGGCGTCATCATGTACACGCTGCTGGCCGGCTCCCCGCCCTTCTGGCA |
| | CCGGAAGCAGATGCTGATGCTGAGCATGATCATGAGCGGCAACTACCAGTTTGGCTCG |
| | CCCGAGTGGGATGATTACTCGGACACCGTGAAGGACCTGGTCTCCCGATTCCTGGTGG |
| | TGCAACCCCAGAACCGCTACACAGCGGAAGAGGCCTTGGCACACCCCTTCTTCCAGCA |
| | GTACTTGGTGGAGGAAGTGCGGCACTTCAGCCCCCGGGGGAAGTTCAAGGTGATCGCT |
| | CTGACCGTGCTGGCTTCAGTGCGGATCTACTACCAGTACCGCCGGGTGAAGCCTGTGA |
| | CCCGGGAGATCGTCATCCGAGACCCCTATGCCCTCCGGCCTCTGCGCCGGCTCATCGA |
| | CGCCTACGCTTTCCGAATCTATGGCCACTGGGTGAAGAAGGGGCAGCAGCAGAACCGG |
| | GCAGCCCTTTTCGAGAACACACCCAAGGCCGTGCTCCTCTCCCTGGCCGAGGAGGACT |
| | ORF Start: ATG at 2 ORF Stop: TGA at 1163 |
| | SEQ ID NO: 44 387 aa MW at 45023.3 kD |
| NOV2d, | MTRDEALPDSHSAQDFYENYEPKEILGRGVSSVVRRCIHKPTSQEYAVKVIDVTGGGS |
| 245245680 | |
| Protein Sequence | FSPEEVRELREATLKEVDTLRKVSGHPNIIQLKDTYETNTFFFLVFDLMKRGELFDYL |
| | TEKVTLSEKETRKIMRALLEVICTLHKLNTVHRDLKPENILLDDNMNIKLTDFGFSCQ |
| | LEPGERLREVCGTPSYLAPEIIECSMNEDHPGYGKEVDMWSTGVIMYTLLAGSPPFWH |
| | RKQMLMLRMIMSGNYQFGSPEWDDYSDTVKDLVSRFLVVQPQNRYTAEEALAHPFFQQ |
| | YLVEEVRHFSPRGKFKVIALTVLASVRIYYQYRRVKPVTREIVIRDPYALRPLRRLID |
| | AYAFRIYGHWVKKGQQQNRAALFENTPKAVLLSLAEEDY |
| | SEQ ID NO: 45 1300 bp |
| NOV2e, | CATGACCCGGGACGAGGCACTGCCGGACTCTCATTCTGCACAGGACTTCTATGAGAAT |
| 245245707 | |
| DNA Sequence | TATGAGCCCAAAGAGATCCTGGGCAGGGGCGTTAGCAGTGTGGTCAGGCGATGCATCC |
| | ACAAGCCCACGAGCCAGGAGTACGCCGTGAAGGTCATCGACGTCACCGGTGGAGGCAG |
| | CTTCAGCCCGGAGGAGGTGCGGGAGCTGCGAGAAGCCACGCTGAAGGAGGTGGACATC |
| | CTGCGCAAGGTCTCAGGGCACCCCAACATCATACAGCTGAAGGACACTTATGAGACCA |
| | ACACTTTCTTCTTCTTGGTGTTTGACCTGATGAAGAGAGGGGAGCTCTTTGACTACCT |
| | CACTCAGAAGGTCACCTTGAGTGACAAGGAAACCAGAAAGATCATGCGAGCTCTGCTG |
| | GAGGTGATCTGCACCTTGCACAAACTCAACATCGTGCACCGGGACCTGAAGCCCGAGA |
| | ACATTCTCTTGGATGACAACATGAACATCAAGCTCACAGACTTTGGCTTTTCCTGCCA |
| | GCTGGAGCCGGGAGAGAGGCTGCGAGTAGAGACAGGGTTTCACCATGTTGGTCAGGCT |
| | GGTCTCAAACTCCTGACCTTACGATCCGCCCGCCTCGGCCTCCCAAAGTGCTGTGATT |
| | ACAGGCGTGAGCCACCATGCCCAGCAGGGCTAGGCATTTCTTCAGAGGTCTGCGGGAC |
| | CCCCAGTTACCTGGCCCCTGAGATTATCGAGTCCTCCATGAATGAGGACCACCCGGGC |
| | TACGGGAAAGAGGTGGACATGTGGAGCACTGGCGTCATCATGTACACGCTGCTGGCCG |
| | GCTCCCCCCCCTTCTGGCACCGGAAGCAGATGCTGATGCTGAGCATGATCATGAGCGG |
| | CAACTACCAGTTTGGCTCGCCCGAGTGGGATGATTACTCGGACACCGTGAAGGACCTG |
| | GTCTCCCGATTCCTGGTGGTGCAACCCCAGAACCGCTACACAGCGGAAGAGGCCTTGG |
| | CACACCCCTTCTTCCAGCAGTACTTGGTGGAGGAAGTGCGGCACTTCAGCCCCCGGGG |
| | GAAGTTCAAGGTGATCGCTCTGACCGTGCTGGCTTCAGTGCGGATCTACTACCAGTAC |
| | CGCCGGGTGAAGCCTGTGACCCGGGAGATCGTCATCCGAGACCCCTATGCCCTCCGGC |
| | CTCTGCGCCGGCTCATCGACGCCTACGCTTTCCGAATCTATGGCCACTGGGTGAAGAA |
| | GGGGCAGCAGCAGAACCGGGCAGCCCTTTTCGAGAACACACCCAAGGCCGTGCTCCTC |
| | TCCCTGGCCGAGGAGGACTACTGA |
| | ORF Start: ATG at 2 ORF Stop: TGA at 1298 |
| | SEQ ID NO: 46 432 aa MW at 49810.8 kD |
| NOV2e, | MTRDEALPDSHSAQDFYENYEPKEILGRGVSSVVRRCIHKPTSQEYAVKVIDVTGGGS |
| 245245707 | |
| Protein Sequence | FSPEEVRELREATLKEVDILRKVSGHPNIIQLKDTYETNTFFFLVFDLMKRGELFDYL |
| | TEKVTLSEKETRKIMRALLEVICTLHKLNIVHRDLKPENILLDDNMNIKLTDFGFSCQ |
| | LEPGERLRVETGFHHVGQAGLKLLTLRSARLGLPKCCDYRREPPCPAGLGISSEVCGT |
| | PSYLAPETIECSMNEDHPGYGKEVDMWSTGVIMYTLLAGSPPFWHRKQMLMLRMIMSG |
| | NYQFGSPEWDDYSDTVKDLVSRFLVVQPQNRYTAEEALAHPFFQQYLVEEVRHFSPRG |
| | KFKVIALTVLASVRIYYQYRRVKPVTREIVIRDPYALRPLRRLIDAYAFRIYGHWVKK |
| | GQQQNRAALFENTPKAVLLSLAEEDY |
| | SEQ ID NO: 47 927 bp |
| NOV2f, | ACCATGGGACATCATCACCACCATCACACCCGGGACGAGGCACTGCCGGACTCTCATT |
| 248494552 | |
| DNA Sequence | CTGCACAGGACTTCTATGAGAATTATGAGCCCAAAGAGATCCTGGGCAGGGGCGTTAG |
| | CAGTGTGGTCAGGCGATGCATCCACAAGCCCACGAGCCAGGAGTACGCCGTGAAGGTC |
| | ATCGACGTCACCGGTGGAGGCAGCTTCAGCCCGGAGGAGGTGCGGGAGCTGCGAGAAG |
| | CCACGCTGAAGGAGGTGGACATCCTGCGCAAGGTCTCAGGCCACCCCAACATCATACA |
| | GCTGAAGGACACTTATGAGACCAACACTTTCTTCTTCTTGGTGTTTGACCTGATGAAG |
| | AGAGGGGAGCTCTTTGACTACCTCACTGAGAAGGTCACCTTGAGTGAGAAGGAAACCA |
| | GAAAGATCATGCGAGCTCTGCTGGAGGTGATCTGCACCTTGCACAAACTCAACATCGT |
| | GCACCGGGACCTGAAGCCCGAGAACATTCTCTTGGATGACAACATGAACATCAAGCTC |
| | ACAGACTTTGGCTTTTCCTGCCAGCTGGAGCCGGGAGAGAGGCTGCGAGAGGTCTGCG |
| | GGACCCCCAGTTACCTGGCCCCTGAGATTATCGAGTGCTCCATGAATGAGGACCACCC |
| | GGGCTACGGGAAAGAGGTGGACATGTGGAGCACTGGCGTCATCATCTACACGCTGCTG |
| | GCCGGCTCCCCGCCCTTCTGGCACCGGAAGCAGATGCTGATGCTGAGGATGATCATGA |
| | GCGGCAACTACCAGTTTGGCTCGCCCGAGTGGGATGATTACTCGGACACCGTCAAGGA |
| | CCTGGTCTCCCGATTCCTGGTGGTGCAACCCCAGAACCGCTACACAGCGGAAGAGGCC |
| | TTGGCACACCCCTTCTTCCAGCAGTACTTGGTGGAGGAAGTGCGGCACTTCAGCTGA |
| | ORF Start: at 1 ORF Stop: TGA at 925 |
| | SEQ ID NO: 48 308 aa MW at 35743.4 kD |
| NOV2f, | TMGHHHHHHTRDEALPDSHSAQDFYENYEPKEILGRGVSSVVRRCIHKPTSQEYAVKV |
| 248494552 | |
| Protein Sequence | IDVTGGGSFSPEEVRELREATLKEVDILRKVSGHPNIIQLKDTYETNTFFFLVFDLMK |
| | RGELFDYLTEKVTLSEKETRKIMRALLEVICTLHKLNIVHRDLKPENILLDDNMNIKL |
| | TDFGFSCQLEPGERLREVCGTPSYLAPEIIECSMNEDHPGYGKEVDMWSTGVIMYTLL |
| | AGSPPFWHRKQMLMLRMIMSGNYQFGSPEWDDYSDTVKDLVSRFLVVQPQNRYTAEEA |
| | LAHPFFQQYLVEEXTRHFS |
| | SEQ ID NO: 49 1194 bp |
| NOV2g, | CGCGGATCCACCATGACCCGGGACGAGGCACTGCCGGACTCTCATTCTGCACAGGACT |
| 242435676 | |
| DNA Sequence | TCTATGAGAATTATGAGCCCAAAGAGATCCTGGGCAGGGGCGTTAGCAGTGTGGTCAG |
| | GCGATGCATCCACAAGCCCACGAGCCAGGAGTACGCCGTGAAGGTCATCGACGTCACC |
| | GGTGGAGGCAGCTTCAGCCCGGAGGAGGTGCGGGAGCTGCGAGAAGCCACGCTGAAGG |
| | AGGTGGACATCCTGCGCAAGGTCTCAGGGCACCCCAACATCATACAGCTGAAGGACAC |
| | TTATGAGACCAACACTTTCTTCTTCTTGGTGTTTGACCTGATGAAGAGAGGGGAGCTC |
| | TTTGACTACCTCACTGAGAAGGTCACCTTGAGTGAGAAGGAAACCAGAAAGATCATGC |
| | GAGCTCTGCTGGAGGTGATCTGCACCTTGCACAAACTCAACATCGTGCACCGGGACCT |
| | GAAGCCCGAGAACATTCTCTTGGATGACAACATGAACATCAAGCTCACAGACTTTGGC |
| | TTTTCCTGCCAGCTGGAGCCGGGAGAGAGGCTGCGAGAGGTCTGCGGGACCCCCAGTT |
| | ACCTGGCCCCTGAGATTATCGAGTGCTCCATGAATGAGGACCACCCGGGCTACGGGAA |
| | AGAGGTGGACATGTGGAGCACTGGCGTCATCATGTACACGCTGCTGGCCGGCTCCCCG |
| | CCCTTCTGGCACCGGAAGCAGATGCTGATGCTGAGGATGATCATGAGCGGCAACTACC |
| | AGTTTGGCTCGCCCGAGTGGGATGATTACTCGGACACCGTGAAGGACCTGGTCTCCCG |
| | ATTCCTGGTGGTGCAACCCCAGAACCGCTACACAGCGGAAGAGGCCTTGGCACACCCC |
| | TTCTTCCAGCAGTACTTGGTGGAGGAAGTGCGGCACTTCAGCCCCCGGGGGAAGTTCA |
| | AGGTGATCGCTCTGACCGTGCTGGCTTCAGTGCGGATCTACTACCAGTACCGCCGGGT |
| | GAAGCCTGTGACCCGGGAGATCGTCATCCGAGACCCCTATGCCCTCCGGCCTCTGCGC |
| | CGGCTCATCGACGCCTACGCTTTCCGAATCTATGGCCACTGGGTGAAGAAGGGGCAGC |
| | AGCAGAACCGGGCAGCCCTTTTCGAGAACACACCCAAGGCCGTGCTCCTCTCCCTGGC |
| | CGAGGAGGACTACTGAGCGGCCGCTTTTTTCCTT |
| | ORF Start: at 1 ORF Stop: TGA at 1174 |
| | SEQ ID NO: 50 391 aa MW at 45424.7 kD |
| NOV2g, | RGSTMTRDEALPDSHSAQDFYENYEPKEILGRGVSSVVRRCIHKPTSQEYAVKVIDVT |
| 242435676 | |
| Protein Sequence | GGGSFSPEEVRELREATLKEVDILRKVSGHPNIIQLKDTYETNTFFFLVFDLMKRGEL |
| | FDYLTEKVTLSEKETRKIMRALLEVICTLHKLNIVHRDLKPENILLDDNMNIKLTDFG |
| | FSCQLEPGERLREVCGTPSYLAPEIIECSMNEDHPGYGKEVDMWSTGVIMYTLLAGSP |
| | PFWHRKQMLMLRMIMSGNYQFGSPEWDDYSDTVKDLVSRFLVVQPQNRYTAEEALAHP |
| | FFQQYLVEEVRHFSPRGKFKVIALTVLASVRIYYQYRRVKPVTREIVIRDPYALRPLR |
| | RLIDAYAFRIYGHWVKKGQQQNRAALFENTPKAVLLSLAEEDY |
| | SEQ ID NO: 51 952 bp |
| NOV2h, | ACATCATCACCACCATCACACCCGGGACGAGGCACTGCCGGACTCTCATTCTGCACAG |
| 254868664 | |
| DNA Sequence | GACTTCTATGAGAATTATGAGCCCAAAGAGATCCTGGGCAGGGGCGTTAGCAGTGTGG |
| | TCAGGCGATGCATCCACAAGCCCACGAGCCAGGAGTACGCCGTGAAGGTCATCGACGT |
| | CACCGGTGGAGGCAGCTTCAGCCCGGAGGAGGTGCGGGAGCTGCGAGAAGCCACGCTG |
| | AAGGAGGTGGACATCCTGCGCAAGGTCTCAGGGCACCCCAACATCATACAGCTGAAGG |
| | ACACTTATGAGACCAACACTTTCTTCTTCTTGGTGTTTGACCTGATGAAGAGAGGGGA |
| | GCTCTTTGACTACCTCACTGAGAAGGTCACCTTGAGTGAGAAGGAAACCAGAAAGATC |
| | ATGCGAGCTCTGCTGGAGGTGATCTGCACCTTGCACAAACTCAACATCGTGCACCGGG |
| | ACCTGAAGCCCGAGAACATTCTCTTGGATCACAACATGAACATCAAGCTCACAGACTT |
| | TGGCTTTTCCTGCCAGCTGGAGCCGGGAGAGAGGCTGCGAGAGGTCTGCGGGACCCCC |
| | AGTTACCTGGCCCCTGAGATTATCGAGTGCTCCATGAATGAGGACCACCCGGGCTACG |
| | GGAAAGAGGTGGACATGTGGAGCACTGGCGTCATCATGTACACGCTGCTGGCCGGCTC |
| | CCCGCCCTTCTGGCACCGGAAGCAGATGCTGATGCTGAGGATGATCATGAGCGGCAAC |
| | TACCAGTTTGGCTCGCCCGAGTGGGATGATTACTCGGACACCGTGAAGGACCTGGTCT |
| | CCCGATTCCTGGTGGTGCAACCCCAGAACCGCTACACAGCGGAAGAGGCCTTGGCACA |
| | CCCCTTCTTCCAGCAGTACTTGGTGGAGGAAGTGCGGCACTTCAGCTGAGCGGCCGCA |
| | CTCGAGCACCACCACCACCACCAC |
| | ORF Start: at 2 ORF Stop: TGA at 917 |
| | SEQ ID NO: 52 305 aa MW at 35454.0 kD |
| NOV2h, | HHHHHHTRDEALPDSHSAQDFYENYEPKEILGRGVSSVVRRCIRKPTSQEYAVKVIDV |
| 254868664 | |
| Protein Sequence | TCGCSFSPEEVRELREATLKEVDILRKVSGHPNITQLKDTYETNTFFFLVFDLMKRGE |
| | LFDYLTEKVTLSEKETRKIMRALLEVICTLHKLNIVHRDLKPENILLDDNMNIKLTDF |
| | GFSCQLEPGERLREVCCTPSYLAPEIIECSMNEDHPGYGKEVDMWSTGVIMYTLLAGS |
| | PPFWHRKQMLMLRMIMSGNYQFGSPEWDDYSDTVKDLVSRFLVVQPQNRYTAEEALAH |
| | PFPQQYLVEEVRHFS |
| | SEQ ID NO: 53 939 bp |
| NOV2i, | CATATGACCCGGGACGAGGCACTGCCGGACTCTCATTCTGCACAGGACTTCTATGAGA |
| 249122191 | |
| DNA Sequence | ATTATGAGCCCAAAGAGATCCTGGGCAGGGGCGTTAGCAGTGTGGTCAGGCGATGCAT |
| | CCACAAGCCCACGAGCCAGGAGTACGCCGTGAAGGTCATCGACGTCACCGGTGGAGGC |
| | AGCTTCAGCCCGGAGGAGGTGCGGGAGCTGCGAGAAGCCACGCTGAAGGAGGTGGACA |
| | TCCTGCGCAAGGTCTCAGGGCACCCCAACATCATACAGCTGAAGGACACTTATGAGAC |
| | CAACACTTTCTTCTTCTTGGTGTTTGACCTGATGAAGAGAGGGGAGCTCTTTGACTAC |
| | CTCACTGAGAAGGTCACCTTGAGTGAGAAGGAAACCAGAAAGATCATGCGAGCTCTGC |
| | TGGAGGTGATCTGCACCTTGCACAAACTCAACATCGTGCACCGGGACCTGAAGCCCGA |
| | GAACATTCTCTTGGATGACAACATGAACATCAAGCTCACAGACTTTGGCTTTTCCTGC |
| | CAGCTGGAGCCGGGAGAGAGGCTGCGAGAGGTCTGCGGGACCCCCAGTTACCTGGCCC |
| | CTGAGATTATCGAGTGCTCCATGAATGAGGACCACCCGGGCTACGGGAAACAGGTGGA |
| | CATGTGGAGCACTGGCGTCATCATGTACACGCTGCTGGCCGGCTCCCCGCCCTTCTGG |
| | CACCGGAAGCAGATGCTGATGCTGAGGATGATCATGAGCGGCAACTACCAGTTTGGCT |
| | CGCCCGAGTGGGATGATTACTCGGACACCGTGAAGGACCTGGTCTCCCGATTCCTGGT |
| | GGTGCAACCCCAGAACCGCTACACAGCGGAAGAGGCCTTGGCACACCCCTTCTTCCAG |
| | CAGTACTTGGTGCAGGAAGTGCGGCACTTCAGCTGAGCGGCCGCACTCGAGCACCACC |
| | ACCACCACCAC |
| | ORF Start: at 1 ORF Stop: TGA at 904 |
| | SEQ ID NO: 54 301 aa MW at 34899.5 kD |
| NOV21, | HMTRDEALPDSHSAQDFYENYEPKEILGRGVSSVVRRCIHKPTSQEYAVKVIDVTGGG |
| 249122191 | |
| Protein Sequence | SFSPEEVRELREATLKEVDILRKVSGHPNIIQLKDTYETNTFFFLVFDLMKRGELFDY |
| | LTEKVTLSEKETRKIMRALLEVICTLHKLNIVHRDLKPENILLDDNMNIKLTDFGFSC |
| | QLEPGERLREVCGTPSYLAPEIIECSMNEDHPGYGKEVDMWSTGVIMYTLLAGSPPFW |
| | HRKQMLMLRMIMSGNYQFGSPEWDDYSDTVKDLVSRFLVVQPQNRYTAEEALAHPFFQ |
| | QYLVEEVRHFS |
| | SEQ ID NO: 55 951 bp |
| NOV2j, | ACCCGGGACGAGGCACTGCCGGACTCTCATTCTGCACAGGACTTCTATGAGAATTATG |
| 249122234 | |
| DNA Sequence | ACCCCAAAGAGATCCTGGGCAGGGGCGTTACCAGTGTGGTCAGGCGATGCATCCACAA |
| | GCCCACGAGCCAGGAGTACGCCGTGAAGGTCATCGACGTCACCGGTGGAGGCAGCTTC |
| | AGCCCGGAGGAGGTGCGGGAGCTGCGAGAAGCCACGCTGAAGGAGGTGGACATCCTGC |
| | GCAAGGTCTCAGGGCACCCCAACATCATACAGCTGAAGGACACTTATGAGACCAACAC |
| | TTTCTTCTTCTTGGTGTTTGACCTGATGAAGAGAGGGGAGCTCTTTGACTACCTCACT |
| | GAGAAGGTCACCTTGAGTGAGAAGGAAACCAGAAAGATCATGCGAGCTCTGCTGGAGG |
| | TGATCTGCACCTTGCACAAACTCAACATCGTGCACCGGGACCTGAAGCCCGAGAACAT |
| | TCTCTTCGATGACAACATGAACATCAAGCTCACAGACTTTGGCTTTTCCTGCCAGCTG |
| | GAGCCGGGAGAGAGGCTGCGAGAGGTCTGCGGGACCCCCAGTTACCTGGCCCCTGAGA |
| | TTATCGAGTGCTCCATGAATGAGGACCACCCGGGCTACGGGAAAGAGGTGGACATGTG |
| | GAGCACTGGCGTCATCATGTACACGCTGCTGGCCGGCTCCCCGCCCTTCTGGCACCGG |
| | AAGCAGATGCTGATGCTGAGGATGATCATGAGCGGCAACTACCAGTTTGGCTCGCCCG |
| | AGTGGGATGATTACTCGGACACCGTCAAGGACCTGGTCTCCCGATTCCTGGTGGTGCA |
| | ACCCCAGAACCGCTACACAGCGGAAGAGGCCTTGGCACACCCCTTCTTCCAGCAGTAC |
| | TTGGTGGAGGAAGTGCGGCACTTCAGCCATCATCACCACCATCACTGAGCGGCCGCAC |
| | TCGAGCACCACCACCACCACCAC |
| | ORF Start: at 1 ORF Stop: TGA at 916 |
| | SEQ ID NO: 56 305 aa MW at 35454.0 kD |
| NOV2j, | TRDEALPDSHSAQDFYENYEPKEILGRGVSSVVRRCIHKPTSQEYAVKVIDVTGGGSF |
| 249122234 | |
| Protein Sequence | SPEEVRELREATLKEVDILRKVSGHPNIIQLKDTYETNTFFFLVFDLMKRGELFDYLT |
| | EKVTLSEKETRKIMRALLEVICTLHKLNIVHRDLKPENILLDDNMNIKLTDFGFSCQL |
| | EPGERLREVCGTPSYLAPEIIECSMNEDHPGYGKEVDMWSTGVIMYTLLAGSPPFWHR |
| | KQMLMLRMIMSGNYQFGSPEWDDYSDTVKDLVSRFLVVQPQNRYTAEEALAHPFFQQY |
| | LVEEVRHFSHHHHHH |
| | SEQ ID NO: 57 1252 bp |
| NOV2k, | CTTTGGGATTCTTGTCAAGCTCCTTCAAGAGCCTGCAAGCACTTAACCAGCCACCCAG |
| CG101996-03 | |
| DNA Sequence | ACTTCCCTCACTGAAGATCTGAGCATGACCCGGGACGAGGCACTGCCGGACTCTCATT |
| | CTGCACAGGACTTCTATGAGAATTATGAGCCCAAAGAGATCCTGGGCAGGGGCGTTAG |
| | CAGTGTGGTCAGGCGATGCATCCACAAGCCCACGAGCCAGGAGTACGCCGTGAAGGTC |
| | ATCGACGTCACCGGTGGAGGCAGCTTCACCCCGGAGGAGGTGCGGGAGCTGCGAGAAG |
| | CCACGCTGAAGGAGGTGGACATCCTGCGCAAGGTCTCAGGGCACCCCAACATCATACA |
| | GCTGAAGGACACTTATGAGACCAACACTTTCTTCTTCTTGGTGTTTGACCTGATGAAG |
| | AGAGGGGAGCTCTTTGACTACCTCACTGAGAAGGTCACCTTGAGTCAGAAGGAAACCA |
| | GAAAGATCATGCGAGCTCTGCTGGAGGTGATCTGCACCTTGCACAAACTCAACATCGT |
| | GCACCGGGACCTGAAGCCCGAGAACATTCTCTTGGATGACAACATGAACATCAAGCTC |
| | ACAGACTTTGGCTTTTCCTGCCAGCTGGAGCCGGGAGAGAGGCTGCGAGAGGTCTGCG |
| | GGACCCCCAGTTACCTGGCCCCTGAGATTATCGAGTGCTCCATGAATGACGACCACCC |
| | GGGCTACGGGAAAGAGGTGGACATGTGGAGCACTGGCGTCATCATGTACACGCTGCTG |
| | GCCGGCTCCCCGCCCTTCTGGCACCGGAAGCAGATCCTGATGCTGAGGATGATCATGA |
| | GCGGCAACTACCAGTTTGGCTCGCCCGAGTGGGATGATTACTCGGACACCGTGAAGGA |
| | CCTGGTCTCCCGATTCCTGGTGGTGCAACCCCAGAACCGCTACACAGCGGAAGAGGCC |
| | TTGGCACACCCCTTCTTCCAGCAGTACTTGGTGGAGGAAGTGCGGCACTTCAGCCCCC |
| | GGGGGAAGTTCAAGGTGATCGCTCTGACCGTGCTGGCTTCAGTGCGGATCTACTACCA |
| | GTACCGCCGGGTGAAGCCTGTGACCCGGGAGATCGTCATCCGAGACCCCTATGCCCTC |
| | CGGCCTCTGCGCCGGCTCATCGACGCCTACGCTTTCCGAATCTATGGCCACTGGGTGA |
| | AGAAGGGGCAGCAGCAGAACCGGGCAGCCCTTTTCGAGAACACACCCAAGGCCGTGCT |
| | CCTCTCCCTGGCCGAGGAGGACTACTGAGGGGCT |
| | ORF Start: ATG at 83 ORF Stop: TGA at 1244 |
| | SEQ ID NO: 58 387 aa MW at 45023.3 kD |
| NOV2k, | MTRDEALPDSHSAQDFYENYEPKEILGRGVSSVVRRCIHKPTSQEYAVKVIDVTGGGS |
| CG101996-03 | |
| Protein Sequence | FSPEEVRELREATLKEVDILRKVSGHPNIIQLKDTYETNTFFFLVFDLMKRGELFDYL |
| | TEKVTLSEKETRKIMRALLEVICTLHKLNIVHRDLKPENILLDDNNNIKLTDFGFSCQ |
| | LEPGERLREVCGTPSYLAPEIIECSMNEDHPGYGKEVDMWSTGVIMYTLLAGSPPFWH |
| | RKQMLMLRMIMSGNYQFGSPEWDDYSDTVKDLVSRFLVVQPQNRYTAEEALAHPFFQQ |
| | YLVEEVRHFSPRGKFKVIALTVLASVRIYYQYRRVKPVTREIVIRDPYALRPLRRLID |
| | AYAFRIYGHWVKKGQQQNRAALFENTPKAVLLSLAEEDY |
| | SEQ ID NO: 59 1194 bp |
| NOV2l, | CGCGGATCCACCATGACCCGGGACGAGGCACTGCCGGACTCTCATTCTGCACAGGACT |
| CG101996-05 | |
| DNA Sequence | TCTATGAGAATTATGAGCCCAAAGAGATCCTGGGCAGGGGCGTTAGCAGTGTGGTCAG |
| | GCGATGCATCCACAAGCCCACGAGCCAGCAGTACGCCGTGAAGGTCATCGACGTCACC |
| | GGTGGAGGCAGCTTCAGCCCGGAGGAGGTGCGGGAGCTGCGAGAAGCCACGCTGAAGG |
| | AGGTGGACATCCTGCGCAAGGTCTCAGGGCACCCCAACATCATACAGCTGAAGGACAC |
| | TTATGAGACCAACACTTTCTTCTTCTTGGTGTTTGACCTGATGAAGAGAGGGGAGCTC |
| | TTTGACTACCTCACTGAGAAGGTCACCTTGAGTGAGAAGGAAACCAGAAAGATCATGC |
| | GAGCTCTGCTGGAGGTGATCTGCACCTTGCACAAACTCAACATCGTGCACCCGGACCT |
| | GAAGCCCGAGAACATTCTCTTGGATGACAACATGAACATCAAGCTCACAGACTTTGGC |
| | TTTTCCTGCCAGCTGGAGCCGGGAGAGAGGCTGCGAGAGGTCTGCGGGACCCCCAGTT |
| | ACCTGGCCCCTGAGATTATCGAGTGCTCCATGAATGAGGACCACCCGGGCTACGGGAA |
| | AGAGGTGGACATGTGGAGCACTGGCGTCATCATGTACACGCTGCTGGCCGGCTCCCCG |
| | CCCTTCTGGCACCGGAAGCAGATGCTGATGCTGAGGATGATCATGAGCGGCAACTACC |
| | AGTTTGGCTCGCCCGAGTGGGATGATTACTCGGACACCGTGAAGGACCTGGTCTCCCG |
| | ATTCCTGGTGGTGCAACCCCAGAACCGCTACACAGCGGAAGAGGCCTTGGCACACCCC |
| | TTCTTCCAGCAGTACTTGGTGGAGGAAGTGCGGCACTTCAGCCCCCGGGGGAAGTTCA |
| | AGGTGATCGCTCTGACCGTGCTGGCTTCAGTGCGGATCTACTACCAGTACCGCCGGGT |
| | GAAGCCTGTGACCCGGGAGATCGTCATCCGAGACCCCTATGCCCTCCGGCCTCTGCGC |
| | CGGCTCATCGACGCCTACGCTTTCCGAATCTATGCCCACTGGGTGAAGAAGGGGCAGC |
| | AGCAGAACCGGGCACCCCTTTTCGAGAACACACCCAAGGCCGTGCTCCTCTCCCTGGC |
| | CGAGGAGGACTACTGAGCGGCCGCTTTTTTCCTT |
| | ORF Start at 1 ORF Stop: TGA at 1174 |
| | SEQ ID NO: 60 391 aa MW at 45424.7 kD |
| NOV21, | RGSTMTRDEALPDSHSAQDFYENYEPKETLGRGVSSVVRRCIHKPTSQEYAVKVIDVT |
| CG101996-05 | |
| Protein Sequence | GGGSFSPEEVRELREATLKEVDILRKVSGHPNIIQLKDTYETNTFFFLVFDLMKRGEL |
| | FDYLTEKVTLSEKETRKIMRALLEVICTLHKLNIVHRDLKPENILLDDNMNIKLTDFG |
| | FSCQLEPGERLREVCGTPSYLAPETIECSMNEDHPGYGKEVDMWSTGVIMYTLLAGSP |
| | PFWHRKQMLMLRMIMSGNYQFGSPEWDDYSDTVKDLVSRFLVVQPQNRYTAEEALAIP |
| | FFQQYLVEEVRHFSPRGKFKVIALTVLASVRIYYQYRRVKPVTREIVIRDPYALRPLR |
| | RLIDAYAFRIYGHWVKKGQQQNRAALFENTPKAVLLSLAEEDY |
| | SEQ ID NO: 61 1165 bp |
| NOV2m, | CATGACCCGGGACGAGGCACTGCCGGACTCTCATTCTGCACAGGACTTCTATGAGAAT |
| CG101996-06 | |
| DNA Sequence | TATGAGCCCAAAGAGATCCTGGGCAGGGGCGTTAGCAGTGTGGTCAGGCGATGCATCC |
| | ACAAGCCCACGAGCCAGGAGTACGCCGTGAAGGTCATCGACGTCACCGGTGGAGGCAG |
| | CTTCAGCCCGGAGGAGGTGCGGGAGCTGCGAGAAGCCACGCTGAAGGACGTGGACATC |
| | CTGCGCAAGGTCTCAGGGCACCCCAACATCATACAGCTGAAGGACACTTATGAGACCA |
| | ACACTTTCTTCTTCTTGGTGTTTGACCTGATGAACAGAGGGGAGCTCTTTGACTACCT |
| | CACTGAGAAGGTCACCTTGAGTGAGAAGGAAACCAGAAAGATCATGCGAGCTCTGCTG |
| | GAGGTGATCTGCACCTTGCACAAACTCAACATCGTGCACCGGGACCTGAAGCCCGACA |
| | ACATTCTCTTGGATGACAACATGAACATCAAGCTCACAGACTTTGGCTTTTCCTGCCA |
| | GCTGGAGCCGGGAGAGAGGCTGCGAGAGGTCTGCGGGACCCCCAGTTACCTGGCCCCT |
| | CAGATTATCGAGTGCTCCATGAATGAGGACCACCCGGGCTACGGGAAAGAGGTGGACA |
| | TCTGGAGCACTGGCGTCATCATGTACACGCTGCTGGCCGGCTCCCCGCCCTTCTGGCA |
| | CCGGAAGCAGATGCTGATGCTGAGGATGATCATGAGCGGCAACTACCAGTTTGGCTCG |
| | CCCGAGTGGGATGATTACTCGGACACCGTGAAGGACCTGGTCTCCCGATTCCTGGTGG |
| | TGCAACCCCAGAACCGCTACACAGCGGAAGAGGCCTTGGCACACCCCTTCTTCCAGCA |
| | GTACTTGGTGGAGGAAGTGCGGCACTTCAGCCCCCGGGGGAAGTTCAAGGTGATCGCT |
| | CTGACCGTGCTGGCTTCAGTGCGGATCTACTACCAGTACCGCCGGGTGAAGCCTGTGA |
| | CCCGGGAGATCGTCATCCGAGACCCCTATGCCCTCCGGCCTCTGCGCCGGCTCATCGA |
| | CGCCTACGCTTTCCGAATCTATGGCCACTGGGTGAAGAAGGGGCAGCAGCAGAACCGG |
| | GCAGCCCTTTTCGAGAACACACCCAAGGCCGTGCTCCTCTCCCTGGCCGAGGAGGACT |
| | ACTGA |
| | ORF Start: ATG at 2 ORF Stop: TGA at 1163 |
| | SEQ ID NO: 62 387 aa MW at 45023.3 kD |
| NOV2m, | MTRDEALPDSHSAQDFYENYEPKEILGRGVSSVVRRCIHKPTSQEYAVKVIDVTGGGS |
| CG101996-06 | |
| Protein Sequence | FSPEEVRELREATLKEVDILRKVSGHPNIIQLKDTYETNTFFFLVFDLMKRGELFDYL |
| | TEKVTLSEKETRKIMRALLEVICTLHKLNIVHRDLKPENILLDDNMNIKLTDFGFSCQ |
| | LEPGERLREVCGTPSYLAPEIIECSMNEDHPGYGKEVDMWSTGVIMYTLLAGSPPFWH |
| | RKQMLMLRMIMSGNYQFGSPEWDDYSDTVKDLVSRFLVVQPQNRYTAEEALAHPFFQQ |
| | YLVEEVRHFSPRGKFKVIALTVLASVRIYYQYRRVKPVTREIVIRDPYALRPLRRLID |
| | AYAFRIYGHWVKKGQQQNRAALFENTPKAVLLSLAEEDY |
| | SEQ ID NO: 63 927 bp |
| NOV2n, | ACCATGGGACATCATCACCACCATCACACCCGGGACGAGGCACTGCCGGACTCTCATT |
| CG101996-07 | |
| DNA Sequence | CTGCACAGGACTTCTATGAGAATTATGAGCCCAAAGAGATCCTGGGCAGGGGCGTTAG |
| | CAGTGTGGTCAGGCGATGCATCCACAAGCCCACGAGCCAGGAGTACGCCGTGAAGGTC |
| | ATCGACGTCACCGGTGGAGGCAGCTTCAGCCCGGAGGAGGTGCGGGAGCTGCGAGAAG |
| | CCACGCTGAAGGAGGTGGACATCCTGCGCAAGGTCTCAGGGCACCCCAACATCATACA |
| | GCTGAAGGACACTTATGAGACCAACACTTTCTTCTTCTTGGTGTTTGACCTGATGAAG |
| | AGAGGGGAGCTCTTTGACTACCTCACTGAGAAGGTCACCTTGAGTGAGAAGGAAACCA |
| | GAAAGATCATGCGAGCTCTGCTGGAGGTGATCTGCACCTTGCACAAACTCAACATCGT |
| | GCACCGGGACCTGAAGCCCGAGAACATTCTCTTGGATGACAACATGAACATCAAGCTC |
| | ACAGACTTTGGCTTTTCCTGCCAGCTGGAGCCGGGAGAGAGGCTGCGAGAGGTCTGCG |
| | GGACCCCCAGTTACCTGGCCCCTGAGATTATCGAGTGCTCCATGAATGAGGACCACCC |
| | GGGCTACGGGAAAGAGGTGGACATGTGGAGCACTGGCGTCATCATGTACACGCTGCTG |
| | GCCGGCTCCCCGCCCTTCTGGCACCGGAAGCAGATGCTGATGCTGAGGATGATCATGA |
| | GCGGCAACTACCAGTTTGGCTCGCCCGAGTGGGATGATTACTCGGACACCGTGAAGGA |
| | CCTGGTCTCCCGATTCCTGGTGGTGCAACCCCAGAACCGCTACACAGCGGAAGAGGCC |
| | TTGGCACACCCCTTCTTCCAGCAGTACTTGGTGGAGGAAGTGCGGCACTTCAGCTGA |
| | ORF Start: at 1 ORF Stop: TGA at 925 |
| | SEQ ID NO: 64 1308 aa MW at 35743.4 kD |
| NOV2n, | TMGHHHHHHTRDEALPDSHSAQDFYENYEPKEILGRGVSSVVRRCIHKPTSQEYAVKV |
| CG101996-07 | |
| Protein Sequence | IDVTGGGSFSPEEVRELREATLKEVDILRKVSGHPNIIQLKDTYETNTFFFLVFDLMK |
| | RGELFDYLTEKVTLSEKETRKIMRALLEVICTLHKLNIVHRDLKPENILLDDNNNIKL |
| | TDFGFSCQLEPGERLREVCGTPSYLAPEIIECSMNEDHPGYGKEVDMWSTGVIMYTLL |
| | AGSPPFWHRKQMLMLRMIMSGNYQFGSPEWDDYSDTVKDLVSRFLVVQPQNRYTAEEA |
| | LAHPFFQQYLVEEVRHFS |
| | SEQ ID NO: 65 924 bp |
| NOV2o, | ACCATGACCCGGGACGAGGCACTGCCGGACTCTCATTCTGCACAGGACTTCTATGAGA |
| CG101996-08 | |
| DNA Sequence | ATTATGAGCCCAAAGAGATCCTGGGCAGGGGCGTTAGCAGTGTGGTCAGGCGATGCAT |
| | CCACAAGCCCACGAGCCAGGAGTACGCCGTGAAGGTCATCGACGTCACCGGTGGAGGC |
| | AGCTTCAGCCCGGAGGAGGTGCGGGAGCTGCGAGAAGCCACGCTGAAGGAGGTGGACA |
| | TCCTGCGCAAGGTCTCAGGGCACCCCAACATCATACAGCTGAAGGACACTTATGAGAC |
| | CAACACTTTCTTCTTCTTGGTGTTTGACCTGATGAAGAGAGGGGAGCTCTTTGACTAC |
| | CTCACTGAGAAGGTCACCTTGAGTGAGAAGGAAACCAGAAAGATCATGCGAGCTCTGC |
| | TGGAGGTGATCTGCACCTTGCACAAACTCAACATCGTGCACCGGGACCTGAAGCCCGA |
| | GAACATTCTCTTGGATGACAACATGAACATCAAGCTCACAGACTTTGGCTTTTCCTGC |
| | CAGCTGGAGCCGGGAGAGAGGCTGCGAGAGGTCTGCGGGACCCCCAGTTACCTGGCCC |
| | CTGAGATTATCGAGTGCTCCATGAATGAGGACCACCCGGGCTACGGGAAAGAGGTGGA |
| | CATGTGGAGCACTGGCGTCATCATGTACACGCTGCTGGCCGGCTCCCCGCCCTTCTGG |
| | CACCGGAAGCAGATGCTGATGCTGAGGATGATCATGAGCGGCAACTACCAGTTTGGCT |
| | CGCCCGAGTGGGATGATTACTCGGACACCGTGAAGGACCTGGTCTCCCGATTCCTGGT |
| | GGTGCAACCCCAGAACCGCTACACAGCGGAAGAGGCCTTGGCACACCCCTTCTTCCAG |
| | CAGTACTTGGTGGAGGAAGTGCGGCACTTCAGCCATCATCACCACCATCACTGA |
| | ORF Start: at 1 ORF Stop: TGA at 922 |
| | SEQ ID NO: 66 307 aa MW at 35686.3 kD |
| NOV2o, | TMTRDEALPDSHSAQDFYENYEPKEILGRGVSSVVRRCIHKPTSQEYAVKVIDVTGGG |
| CG101996-08 | |
| Protein Sequence | SFSPEEVRELREATLKEVDILRKVSGHPNIIQLKDTYETNTFFFLVFDLMKRGELFDY |
| | LTEKVTLSEKETRKIMRALLEVICTLHKLNIVHRDLKPENILLDDNMNIKLTDFGFSC |
| | QLEPGERLREVCGTPSYLAPEIIECSMNEDHPGYGKEVDMWSTGVIMYTLLAGSPPFW |
| | HRKQMLMLRMIMSGNYQFGSPEWDDYSDTVKDLVSRFLVVQPQNRYTAEEALAHPFFQ |
| | QYLVEEVRHFSHHHHHH |
| | SEQ ID NO: 67 939 bp |
| NOV2p, | CATATGACCCGGGACGAGGCACTGCCGGACTCTCATTCTGCACAGGACTTCTATGAGA |
| CG101996-09 | |
| DNA Sequence | ATTATGAGCCCAAAGAGATCCTGGGCAGGGGCGTTAGCAGTGTGGTCAGGCGATGCAT |
| | CCACAAGCCCACGAGCCAGGAGTACGCCGTGAAGGTCATCGACGTCACCGGTGGAGGC |
| | ACCTTCACCCCCCACCACCTCCCCCACCTCCCACAACCCACCCTCAACCACCTCCACA |
| | AGCTTCAGCCCGGAGGAGGTGCGGGAGCTGCGAGAAGCCACGCTGAAGGAGGTGGACA |
| | TCCTGCGCAAGGTCTCAGGGCACCCCAACATCATACAGCTGAAGGACACTTATGAGAC |
| | CAACACTTTCTTCTTCTTGGTGTTTGACCTGATGAAGAGAGGGGAGCTCTTTGACTAC |
| | CTCACTGAGAAGGTCACCTTGAGTGAGAAGGAAACCAGAAAGATCATGCGAGCTCTGC |
| | TGGAGGTGATCTGCACCTTGCACAAACTCAACATCGTGCACCGGGACCTGAAGCCCGA |
| | GAACATTCTCTTGGATGACAACATGAACATCAAGCTCACAGACTTTGGCTTTTCCTGC |
| | CAGCTGGAGCCGGGAGAGAGGCTGCGAGAGGTCTGCGGGACCCCCAGTTACCTGGCCC |
| | CTGAGATTATCGAGTGCTCCATGAATGAGGACCACCCGGGCTACGGGAAAGAGGTGGA |
| | CATGTGGACCACTGGCGTCATCATGTACACGCTGCTGGCCGGCTCCCCGCCCTTCTGG |
| | CACCGGAAGCAGATGCTGATGCTGAGGATGATCATGAGCGGCAACTACCAGTTTGGCT |
| | CGCCCGAGTGGGATGATTACTCGGACACCGTGAAGGACCTGGTCTCCCGATTCCTGGT |
| | GGTGCAACCCCAGAACCGCTACACAGCGGAAGAGGCCTTGGCACACCCCTTCTTCCAG |
| | CAGTACTTGGTGGAGGAAGTGCGGCACTTCAGCTGAGCGGCCGCACTCGAGCACCACC |
| | ACCACCACCAC |
| | ORF Start: at 1 ORF Stop: TGA at 904 |
| | SEQ ID NO: 68 301 aa MW at 34899.5 kD |
| NOV2p, | HMTRDEALPDSHSAQDFYENYEPKEILGRGVSSVVRRCIHKPTSQEYAVKVIDVTGGG |
| CG101996-09 | |
| Protein Sequence | SFSPEEVRELREATLKEVDILRKVSGHPNIIQLKDTYETNTFFFLVFDLMKRGELFDY |
| | LTEKVTLSEKETRKIMRALLEVICTLHKLNIVHRDLKPENILLDDNNNIKLTDFGFSC |
| | QLEPGERLREVCGTPSYLAPEIIECSMNEDHPGYGKEVDMWSTGVIMYTLLAGSPPFW |
| | HRKQMLMLRMIMSGNYQFGSPEWDDYSDTVKDLVSRFLVVQPQNRYTAEEALAHPFFQ |
| | QYLVEEVRHFS |

Sequence comparison of the above protein sequences yields the following sequence relationships shown in Table 2B.

TABLE 2B
Comparison of NOV2a against NOV2b through NOV2p.

| Protein | NOV2a Residues/ | Identities/Similarities |
|---|---|---|
| Sequence | Match Residues | for the Matched Region |
| NOV2b | 1 . . . 152 | 152/152 (100%) |
| | 281 . . . 432 | 152/152 (100%) |
| NOV2c | 1 . . . 152 | 152/152 (100%) |
| | 236 . . . 387 | 152/152 (100%) |
| NOV2d | 1 . . . 152 | 152/152 (100%) |
| | 236 . . . 387 | 152/152 (100%) |
| NOV2e | 1 . . . 152 | 152/152 (100%) |
| | 281 . . . 432 | 152/152 (100%) |
| NOV2f | 1 . . . 65 | 65/65 (100%) |
| | 244 . . . 308 | 65/65 (100%) |
| NOV2g | 1 . . . 152 | 152/152 (100%) |
| | 240 . . . 391 | 152/152 (100%) |
| NOV2h | 1 . . . 65 | 65/65 (100%) |
| | 241 . . . 305 | 65/65 (100%) |
| NOV2i | 1 . . . 65 | 65/65 (100%) |
| | 237 . . . 301 | 65/65 (100%) |
| NOV2j | 1 . . . 65 | 65/65 (100%) |
| | 235 . . . 299 | 65/65 (100%) |
| NOV2k | 1 . . . 152 | 152/152 (100%) |
| | 236 . . . 387 | 152/152 (100%) |
| NOV2l | 1 . . . 152 | 152/152 (100%) |
| | 240 . . . 391 | 152/152 (100%) |
| NOV2m | 1 . . . 152 | 152/152 (100%) |
| | 236 . . . 387 | 152/152 (100%) |
| NOV2n | 1 . . . 65 | 65/65 (100%) |
| | 244 . . . 308 | 65/65 (100%) |
| NOV2o | 1 . . . 65 | 65/65 (100%) |
| | 237 . . . 301 | 65/65 (100%) |
| NOV2p | 1 . . . 65 | 65/65 (100%) |
| | 237 . . . 301 | 65/65 (100%) |

Further analysis of the NOV2a protein yielded the following properties shown in Table 2C.

TABLE 2C
Protein Sequence Properties NOV2a

| | |
|---|---|
| PSort | 0.5098 probability located in microbody (peroxisome); |
| analysis: | 0.4500 probability located in cytoplasm; 0.3051 |
| | probability located in lysosome (lumen); 0.1000 |
| | probability located in mitochondrial matrix space |
| SignalP | No Known Signal Sequence Predicted |
| analysis: | |

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | | Residues/ | Similarities | |
| Geneseq | Protein/Organism/Length [Patent | Match | for the | Expect |
| Identifier | #, Date] | Residues | Matched Region | Value |
| ABB09290 | Human phosphorylase kinase | 1 . . . 140 | 82/140 (58%) | 5e−43 |
| | gamma 2 (PHKG2) protein SEQ ID | 239 . . . 378 | 105/140 (74%) | |
| | NO: 4 - Homo sapiens, 406 aa. | | | |
| | [WO200194365-A2, 13 DEC. 2001] | | | |
| AAY43921 | Rabbit protein kinase #3 - | 1 . . . 56 | 55/56 (98%) | 2e−26 |
| | Oryctolagus cuniculus, 268 aa. | 213 . . . 268 | 55/56 (98%) | |
| | [US5958784-A, 28 SEP. 1999] | | | |
| AAY43922 | Mouse protein kinase #3 - Mus sp, | 1 . . . 56 | 50/56 (89%) | 2e−23 |
| | 268 aa. [US5958784-A. 28 SEP. | 213 . . . 268 | 53/56 (94%) | |
| | 1999] | | | |
| ABG10311 | Novel human diagnostic protein | 44 . . . 140 | 49/104 (47%) | 1e−19 |
| | #10302 - Homo sapiens, 886 aa. | 615 . . . 718 | 69/104 (66%) | |
| | [WO200175067-A2, 11 OCT. 2001] | | | |
| ABB58577 | Drosophila melanogaster | 64 . . . 147 | 43/84 (51%) | 4e−17 |
| | polypeptide SEQ ID NO 2523 - | 470 . . . 553 | 57/84 (67%) | |
| | Drosophila melanogaster, 560 aa. | | | |
| | [WO200171042-A2, 27 SEP. 2001] | | | |

In a BLAST search of public sequence datbases, the NOV2a protein was found to have homology to the proteins shown in the BLASTP data in Table 2E.

TABLE 2E
Public BLASTP Results for NOV2a

| | | NOV2a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q16816 | Phosphorylase B kinase gamma | 1 . . . 152 | 152/152 (100%) | 5e−84 |
| | catalytic chain, skeletal muscle | 235 . . . 386 | 152/152 (100%) | |
| | isoform (EC 2.7.1.38) | | | |
| | (Phosphorylase kinase gamma | | | |
| | subunit 1) - Homo sapiens (Human), | | | |
| | 386 aa. | | | |
| KIRBFG | phosphorylase kinase (EC 2.7.1.38) | 1 . . . 152 | 147/152 (96%) | 1e−81 |
| | catalytic chain, skeletal muscle - | 236 . . . 387 | 149/152 (97%) | |
| | rabbit, 387 aa. | | | |
| P00518 | Phosphorylase B kinase gamma | 1 . . . 152 | 147/152 (96%) | 1e−81 |
| | catalytic chain, skeletal muscle | 235 . . . 386 | 149/152 (97%) | |
| | isoform (EC 2.7.1.38) | | | |
| | (Phosphorylase kinase gamma | | | |
| | subunit 1) - Oryctolagus cuniculus | | | |
| | (Rabbit), 386 aa. | | | |
| S00731 | phosphorylase kinase (EC 2.7.1.38) | 1 . . . 151 | 142/151 (94%) | 3e−78 |
| | catalytic chain [similarity] - rat, 388 | 236 . . . 386 | 147/151 (97%) | |
| | aa. | | | |
| P13286 | Phosphorylase B kinase gamma | 1 . . . 151 | 142/151 (94%) | 3e−78 |
| | catalytic chain, skeletal muscle | 235 . . . 385 | 147/151 (97%) | |
| | isoform (EC 2.7.1.38) | | | |
| | (Phosphorylase kinase gamma | | | |
| | subunit 1) - Rattus norvegicus (Rat), | | | |
| | 387 aa. | | | |

PFam analysis predicts that the NOV2a protein contains the domains shown in the Table 2F.

TABLE 2F
Domain Analysis of NOV2a

| Pfam | NOV2a | Identities/Similarities | Expect |
|---|---|---|---|
| Domain | Match Region | for the Matched Region | Value |
| pkinase | 3 . . . 53 | 16/54 (30%) | 4.4e−09 |
| | | 43/54 (80%) | |

### Example 3

The NOV3 clone was analyzed, and the nucleotide and encoded polypeptide sequences are shown in Table 3A.

TABLE 3A
NOV3 Sequence Analysis

| | |
|---|---|
| | SEQ ID NO: 69 2727 bp |
| NOV3a, | AGAAGAGCGGAGCTGTGAGCAGTACTGCGGCCTCCTCTCCTCTCCTAACCTCGCTCTC |
| CG102822-01 | |
| DNA Sequence | GCGGCCTAGCTTTACCCGCCCGCCTGCTCGGCGACCAGAACACCTTCCACCATGACCA |
| | CCTCAGCAAGTTCCCACTTAAATAAAGGCATCAAGCAGGTGTACATGTCCCTGCCTCA |
| | GGGTGAGAAAGTCCAGGCCATGTATATCTGGATCGATGGTACTGGAGAAGGACTGCGC |
| | TGCAAGACCCGGACCCTGGACAGTGAGCCCAAGTGTGTGGAAGAGTTGCCTGAGTGGA |
| | ATTTCGATGGCTCTAGTACTTTACAGTCTGAGGGTTCCAACAGTGACATGTATCTCGT |
| | GCCTGCTGCCATGTTTCGGGACCCCTTCCGTAAGGACCCTAACAAGCTGGTGTTATGT |
| | GAAGTTTTCAAGTACAATCGAAGGCCTGCAGAGACCAATTTGAGGCACACCTGTAAAC |
| | GGATAATGGACATGGTGAGCAACCAGCACCCCTGGTTTGGCATGGAGCAGGAGTATAC |
| | CCTCATGGGGACAGATGGGCACCCCTTTCGTTGGCCTTCCAACGGCTTCCCAGGGCCC |
| | CAGGGTCCATATTACTGTGGTGTGGGAGCAGACAGAGCCTATGGCAGGGACATCGTGG |
| | AGGCCCATTACCGGGCCTGCTTGTATGCTGGAGTCAAGATTGCGGGGACTAATGCCGA |
| | GGTCATGCCTGCCCAGTGGGAATTTCAGATTGGACCTTGTGAAGGAATCAGCATGGGA |
| | GATCATCTCTGGGTGGCCCGTTTCATCTTCCATCGTGTGTGTGAAGACTTTGGAGTGA |
| | TAGCAACCTTTGATCCTAAGCCCATTCCTGGGAACTGGAATGGTGCAGGCTGCCATAC |
| | CAACTTCAGCACCAAGGCCATGCGGGAGGAGAATGGTCTGAAGTACATCGAGGAGGCC |
| | ATTGAGAAACTAACCAAGCGGCACCAGTACCACATCCGTGCCTATGATCCCAAGGGAG |
| | GCCTGGACAATGCCCGACGTCTAACTGGATTCCATGAAACCTCCAACATCAACGACTT |
| | TTCTGCTGGTGTAGCCAATCGTAGCGCCAGACTACGCATTCCCCGGACTGTTGGCCAG |
| | GAGAAGAAGGGTTACTTTGAAGATCGTCGCCCCTCTGCCAACTGCGAGCCCTTTTCGG |
| | TGACAGAAGCCCTCATCCGCACGTGTCTTCTCAATGAAACCGGCGATGAGCCCTTCCA |
| | GTACAAAAATTAAGTGGACTAGACCTCCAGCTGTTGAGCCCCTCCTAGTTCTTCATCC |
| | CTGACTCCAACTCTTCCCCCTCTCCCAGTTGTCCCGATTGTAACTCAAAGGGTGGAAT |
| | ATCAAGGTCGTTTTTTTCATTCCATGTGCCCAGTTAATCTTGCTTTCTTTTGTTTGGC |
| | TGGGATAGAGGGGTCAAGTTATTAATTTCTTCACACCTACCCTCCTTTTTTTCCCTAT |
| | CACTGAAGCTTTTTAGTGCATTAGTGGGGAGGAGGGTGGGGAGACATAACCACTGCTT |
| | CCATTTAATGGGGTGCACCTGTCCAATAGGCGTACGTATCCGGACAGAGCACGTTTGC |
| | AGAGGGGTCTCTCTCCAGGTAGCTGAAAGGGAAGACCTGACGTACTCTGGTTAGGTTA |
| | GGACTTGCCCTCGTGGTGGAAACTTTTCTTAAAAAGTTATAACCAACTTTTCTATTAA |
| | AAGTGGGAATTAGGAGAGAAGGTAGGGGTTGGGAATCAGAGAGAATGGCTTTGGTCTC |
| | TTGCTTGTGGGACTAGCCTGGCTTGGGACTAAATGCCCTGCTCTGAACACAAGCTTAG |
| | TATAAACTGATGGATATCCCTACCTTGAAAGAAGAAAAGGTTCTTACTGCTTGGTCCT |
| | TGATTTATCACACAAAGCAGAATAGTATTTTTATATTTAAATGTAAAGACAAAAAACT |
| | ATATGTATGGTTTTGTGGATTATGTGTGTTTTGGCTAAAGGAAAAAACCATCCAGGTC |
| | ACGGGGCACCAAATTTGAGACAAATAGTCGGATTAGAAATAAAGCATCTCATTTTGAG |
| | TAGAGAGCAAGGAAGTGGTTCTTACATGGTGATCTGGGATTACGCCCTCAAGACCCCT |
| | TTTGGGTTTCTGCCCTGCCCACCCTCTGGAGAAGGTGGCACTGATTAGTTAACAGACC |
| | AACACCGTTACTAGCAGTCACTGATCTCCGTGGCTTTGGTTTAAAAGACACACTTGTC |
| | CACATAGGTTTAGAGATAAGAGTTGGCTGGTCAACTTGAGCATGTTACTGACAGAGGG |
| | GGTATTGGGGTTATTTTCTGGTAGGAATAGCATGTCACTAAAGCAGGCCTTTGATATT |
| | AAATTTTTTAAAAAGCAAAATTATAGAAGTTTAGATTTTAATCAAATTTGTAGCGTTT |
| | CTAGGTATTTACAGATGCTGTTGCTCAACGTCTCCTACCTCTGCTCTGAGAGATGGGA |
| | CAGGCTGAGTCAAACACTGTAATTTTGTATCTTGATGTCTTTGTTAAGACTGCTGAAG |
| | AATTATTTTTTCTTTTATAATAAGGAATAAACCCCACCTTTATTCCTTCATTTCATCT |
| | ACCATTTTCTGGTTCTTGTGTTGGCTGTGGCAGGCCAGCTGTGGTTTTCTTTTGCCAT |
| | GACAACTTCTAATTGCCATGTACAGTATGTTCAAAGTCPAATAACTCCTCATTGTAAA |
| | CAAACTGTGTAACTGCCCAAAGCAGCACTTATAAATCACCCTAACATAAAAAAAAAAA |
| | A |
| | ORF Start: at 68 ORF Stop: TAA at 1229 |
| | SEQ ID NO: 70 387 aa MW at 43593.8kD |
| NOV3a, | LYPPACSATRTPSTMTTSASSHLNKGTKQVYMSLPQGEKVQANYIWIDGTGEGLRCKT |
| CG102822-01 | |
| Protein Sequence | RTLDSEPKCVEELPEWNFDGSSTLQSEGSNSDMYLVPAAMFRDPFRKDPNKLVLCEVF |
| | KYNRRPAETNLRHTCKRIMDMVSNQHPWFGMEQEYTLMGTDGHPFGWPSNGFPGPQGP |
| | YYCGVGADRAYCRDIVEAHYRACLYAGVKIAGTNAEVMPAQWEFQIGPCEGISMGDHL |
| | WVARFILHRVCEDFGVIATFDPKPIPGNNNGAGCHTNFSTKAMREENGLKYIEEAIEK |
| | LSKRHQYHIRAYDPKGGLDNARRLTGFHETSNINDFSAGVANRSARLRIPRTVGQEKK |
| | GYFEDRRPSANCEPFSVTEALIRTCLLNETGDEPFQYKN |
| | SEQ ID NO: 71 1366 bp |
| NOV3b, | CGCGAGAGCAGGTTAGGAGAGGAGAGGAGGCCGCAGTACTGCTCACACGCTCCGCTCT |
| CG102822-03 | |
| DNA Sequence | TCTCCCACTCTCGGCCTACCTTTACCCGCCCGCCTGCTCGGCGACCAGAACACCTTCC |
| | ACCATGACCACCTCAGCAAGTTCCCACTTAAATAAAGGCATCAAGCAGGTGTACATGT |
| | CCCTGCCTCAGGGTGAGAAAGTCCAGGCCATGTATATCTGGATCGATGGTACTGGAGA |
| | AGGACTGCGCTCCAAGACCCGGACCCTGGACAGTGAGCCCAAGTGTGTGGAAGAGTTG |
| | CCTGAGTGGAATTTCGATGGCTCCAGTACTTTACAGTCTGAGGGTTCCAACAGTGACA |
| | TGTATCTCGTGCCTGCTGCCATGTTTCGGGACCCCTTCCGTAAGGACCCTAACAAGCT |
| | GGTGTTATGTGAAGTTTTCAAGTACAATCGAAGGCCTGCAGAGACCAATTTGAGGCAC |
| | ACCTGTAAACGGATAATGGACATGGTGAGCAACCAGCACCCCTGGTTTGGCATGGAGC |
| | AGGAGTATACCCTCATGGGGACAGATGGGCACCCCTTTGGTTGGCCTTCCAACGGCTT |
| | CCCAGCGCCCCAGGGTCCATATTACTGTGGTGTGGGAGCAGACAGAGCCTATGGCAGG |
| | GACATCGTGGAGGCCCATTACCGGGCCTGCTTGTATGCTGCAGTCAAGATTGCGGGGA |
| | CTAATGCCGAGGTCATGCCTGCCCAGTGGGAATTTCAGATTGGACCTTGTGAAGGAAT |
| | CAGCATGGGAGATCATCTCTGGCTGGCCCGTTTCATCTTGCATCGTGTGTGTGAAGAC |
| | TTTGGAGTGATAGCAACCTTTGATCCTAAGCCCATTCCTGGGAACTGGAATGGTGCAG |
| | CCTGCCATACCAACTTCAGCACCAAGGCCATGCGGGAGGAGAATCGTCTGAAGTACAT |
| | CGAGGAGGCCATTGAGAAACTAAGCAAGCGCCACCAGTACCACATCCGTGCCTATGAT |
| | CCCAAGGGAGGCCTGGACAATGCCCGACGTCTAACTGGATTCCATGAAACCTCCAACA |
| | TCAACGACTTTTCTGGTGGTGTAGCCAATCGTAGCGCCAGCATACGCATTCCCCGGAC |
| | TGTTGGCCAGGAGAAGAAGGGTTACTTTGAAGATCGTCGCCCCTCTGCCAACTGCGAC |
| | CCCTTTTCGGTGACAGAAGCCCTCATCCGCACGTGTCTTCTCAATGAAACCGGCGATG |
| | AGCCCTTCCAGTACAAAAATTAAGTGGACTAGACCTCCAGCTGTTGAGCCCCTCCTAG |
| | TTCTTCATCCCACTCCAACTCTTCCCCCTCTCCCAGTTGTCCCGATTGTAACTCAAAG |
| | GGTGGAATATCAAGGTCCTTTTTTTTCATTCC |
| | ORF Start: ATG at 120 ORF Stop: TAA at 1239 |
| | SEQ ID NO: 72 373 aa MW at 42050.0kD |
| NOV3b, | MTTSASSHLNKGIKQVYMSLPQGEKVQANYIWIDGTGEGLRCKTRTLDSEPKCVEELP |
| CG102822-03 | |
| Protein Sequence | EWNFDGSSTLQSEGSNSDMYLVPAANFRDPFRKDPNKLVLCEVFKYNRRPAETNLRHT |
| | CKRIMDMVSNQHPWFGMEQEYTLMGTDGHPFGWPSNGFPGPQGPYYCGVGADRAYGRD |
| | IVEAHYRACLYAGVKIAGTNAEVMPAQWEFQIGPCEGISMGDHLWVARFILHRVCEDF |
| | GVIATFDPKPIPGNWNGAGCHTNFSTKAMREENGLKYIEEAIEKLSKRHQYHIRAYDP |
| | KGGLDNARRLTGFHETSNINDFSGGVANRSASIRIPRTVGQEKKGYFEDRRPSANCDP |
| | FSVTEALIRTCLLNETGDEPFQYKN |
| | SEQ ID NO: 73 2631 bp |
| NOV3c, | ATGACCACCTCAGCAAGTTCCCACTTAAATAAAGGCATCAAGCAGGTGTACATGTCCC |
| CG102822-02 | |
| DNA Sequence | TGCCTCAGGGTGAGAAAGTCCAGGCCATCTATATCTCGATCGATGGTACTGGAGAAGG |
| | ACTGCGCTCCAAGACCCGGACCCTGGACAGTGAGCCCAAGTGTGTGGAAGAGTTGCCT |
| | GAGTGGAATTTCGATGGCTCTAGTACTTTACAGTCTGAGGGTTCCAACAGTGACATGT |
| | ATCTCGTGCCTGCTGCCATGTTTCGGGACCCCTTCCGTAAGGACCCTAACAAGCTGGT |
| | GTTATGTGAAGTTTTCAAGTACAATCGAAGGCCTGCACAGACCAATTTGAGGCACACC |
| | TGTAAACGGATAATGGACATGGTGAGCAACCAGCACCCCTGGTTTGGCATGGAGCAGG |
| | AGTATACCCTCATGGGGACAGATGGGCACCCCTTTGGTTGGCCTTCCAACGGCTTCCC |
| | AGGGCCCCAGGGTCCATATTACTGTGGTGTGGGAGCAGACAGAGCCTATGGCAGGGAC |
| | ATCGTGCAGGCCCATTACCGGCCCTGCTTGTATGCTGGAGTCAAGATTGCGGGGACTA |
| | ATGCCGAGGTCATGCCTGCCCAGTCGGAATTTCAGATTGGACCTTGTGAAGGAATCAG |
| | CATGGGAGATCATCTCTGGGTGGCCCGTTTCATCTTGCATCGTGTGTGTGAAGACTTT |
| | GGAGTGATAGCAACCTTTGATCCTAAGCCCATTCCTGGGAACTGGAATGGTGCAGGCT |
| | GCCATACCAACTTCAGCACCAAGGCCATGCGGGAGGAGAATGGTCTGAAGTACATCGA |
| | GGAGGCCATTGAGAAACTAAGCAAGCGGCACCAGTACCACATCCGTGCCTATCATCCC |
| | AAGGGAGGCCTGGACAATGCCCGACGTCTAACTGGATTCCATGAAACCTCCAACATCA |
| | ACGACTTTTCTGCTGGTGTAGCCAATCGTAGCGCCAGCATACGCATTCCCCGGACTGT |
| | TGGCCAGGAGAAGAAGGGTTACTTTGAAGATCGTCGCCCCTCTGCCAACTGCGACCCC |
| | TTTTCGGTGACAGAAGCCCTCATCCGCACGTGTCTTCTCAATGAAACCGGCGATGAGC |
| | CCTTCCAGTACAAAAATTAAGTGGACTAGACCTCCAGCTGTTGAGCCCCTCCTAGTTC |
| | TTCATCCCACTCCAACTCTTCCCCCTCTCCCAGTTGTCCCGATTGTAACTCAAAGCGT |
| | GGAATATCAAGGTCGTTTTTTTCATTCCATGTGCCCAGTTAATCTTGCTTTCTTTGTT |
| | TGGCTGGGATAGAGGGGTCAAGTTATTAATTTCTTCACACCTACCCTCCTTTTTTTCC |
| | CTATCACTGAAGCTTTTTAGTGCATTAGTGGGGAGGAGGGTGGGGAGACATAACCACT |
| | GCTTCCATTTAATGGGGTGCACCTGTCCAATAGGCGTAGCTATCCGGACAGAGCACGT |
| | TTGCAGAAGGGGGTCTCTTCTTCCAGGTAGCTGAAAGGGCAAGACCTGACGTACTCTG |
| | GTTAGGTTAGGACTTGCCCTCGTGGTGGAAACTTTTCTTAAAAAGTTATAACCAACTT |
| | TTCTATTAAAAGTGGGAATTAGGAGAGAAGGTAGGGGTTGGGAATCAGAGAGAATGGC |
| | TTTCGTCTCTTGCTTGTGGGACTAGCCTGGCTTGGGACTAAATGCCCTGCTCTGAACA |
| | CGAAGCTTAGTATAAACTGATGGATATCCCTACCTTGAAAGAAGAAAAGGTTCTTACT |
| | GCTTGGTCCTTGATTTATCACACAAAGCAGAATAGTATTTTTATATTTAAATGTAAAG |
| | ACAAAAAACTATATGTATGGTTTTGTGGATTATGTGTGTTTTGCTAAAGGAAAAAACC |
| | ATCCAGGTCACGGGGCACCAAATTTGAGACAAATAGTCGGATTAGAAATAAAGCATCT |
| | CATTTTGAGTAGAGAGCAAGGGAAGTGGTTCTTAGATGGTGATCTGGGATTAGGCCCT |
| | CAAGACCTTTTGGGTTTCTGCCCTGCCCACCCTCTGGAGAAGGTGGGCACTCGATTAG |
| | TTAACAGACAACACGTTACTAGCAGTCACTTGATCTCCGTGGCTTTGGTTTAAAAGAC |
| | ACACTTCTCCACATAGGTTTAGAGATAAGAGTTGGCTGGTCAACTTGAGCATGTTACT |
| | GACAGAGGGGGTATTGGGGTTATTTTCTGGTAGGAATAGCATGTCACTAAAGCAGGCC |
| | TTTTGATATTAAATTTTTTAAAAAGCAAAATTATAGAAGTTTAGATTTTAATCAAATT |
| | TGATGGGTTTCTAGGTAATTTTTACAGAATTGCTTGTTTGCTTCAACTGTCTCCTACC |
| | TCTGCCTCTTGGAGGAGATGGGACAGGGCTGGAGTCAAAACACTTGTAATTTTGTATC |
| | TTGATGTCTTTGTTAAGACTGCTGAAGAATTATTTTTTTTCTTTTATAATAAGGAATA |
| | AACCCCACCTTTATTCCTTCATTTCATCTACCATTTTCTGGTTCTTGTGTTGGCTGTG |
| | GCAGGCCAGCTGTGGTTTTCTTTTGCCATGACAACTTCTAATTGCCATGTACAGTATG |
| | TTCAAAGTCAAATAACTCCTCATTGTAAACAAACTGTGTAACTGCCCAAAGCAGCACT |
| | TATAAATCAGCCTAACATAAG |
| | ORF Start: ATG at 1 ORF Stop: TAA at 1120 |
| | SEQ ID NO: 74 373 aa MW at 42064.0kD |
| NOV3c, | MTTSASSHLNKGIKQVYMSLPQGEKVQAMYIWIDGTGEGLRCKTRTLDSEPKCVEELP |
| CG102822-02 | |
| Protein Sequence | EWNFDGSSTLQSEGSNSDMYLVPAANFRDPFRKDPNKLVLCEVFKYNRRPAETNLRHT |
| | CKRIMDMVSNQHPWFGMEQEYTLMGTDGHPFGWPSNGFPGPQGPYYCGVGADRAYGRD |
| | IVEAHYRACLYAGVKIAGTNAEVMPAQWEFQIGPCEGISMGDHLWVARFILHRVCEDF |
| | GVIATFDPKPIPGNWNGAGCHTNFSTKAMREENGLKYIEEAIEKLSKRHQYHIRAYDP |
| | KGGLDNARRLTGFHETSNINDFSAGVANRSASIRIPRTVGQEKKGYFEDRRPSANCDP |
| | FSVTEALIRTCLLNETGDEPFQYKN |
| | SEQ ID NO: 75 2775 bp |
| NOV3d, | GGCACGAGGGAAGAGCGGAGCGTGTGAGCAGTACTGCGGCCTCCTCTCCTCTCCTAAC |
| CG102822-04 | |
| DNA Sequence | CTCGCTCTCGCGGCCTACCTTTACCCGCCCGCCTGCTCGGCGACCAGAACACCTTCCA |
| | CCATGACCACCTCAGCAAGTTCCCACTTAAATAAAGGCATCAAGCAGGTGTACATGTC |
| | CCTGCCTCAGGGTGAGAAAGTCCAGGCCATGTATATCTGGATCGATGGTACTGGAGAA |
| | GGACTGCGCTGCAAGACCCGGACCCTGGACAGTGAGCCCAAGTGTGTGGAAGAGTTGC |
| | CTGAGTGGAATTTCGATGGCTCCAGTACTTTACAGTCTGAGGGTTCCAACAGTGACAT |
| | GTATCTCGTGCCTGCTGCCATGTTTCGGGACCCCTTCCGTAAGGACCCTAACAAGCTG |
| | GTGTTATGTGAAGTTTTCAAGTACAATCGAAGGCCTGCAGAGACCAATTTGAGGCACA |
| | CCTGTAAACGGATAATGGACATGGTGAGCAACCAGCACCCCTGGTTTGGCATGGAGCA |
| | GGAGTATACCCTCATGGGGACAGATGGGCACCCCTTTGGTTGGCCTTCCAACGGCTTC |
| | CCAGGGCCCCAGGGTCCATATTACTGTGGTGTGGGAGCAGACAGAGCCTATGGCAGGG |
| | ACATCGTGGAGGCCCATTACCGGGCCTGCTTGTATGCTGGAGTCAAGATTGCGGGGAC |
| | TAATGCCGAGGTCATGCCTGCCCAGTGGGAATTTCAGATTGGACCTTGTGAAGGAATC |
| | AGCATGGGAGATCATCTCTGGGTGGCCCGTTTCATCTTGCATCGTGTGTGTGAAGACT |
| | TTGGAGTGATAGCAACCTTTGATCCTAAGCCCATTCCTGGGAACTGGAATGGTGCAGG |
| | CTGCCATACCAACTTCAGCACCAAGGCCATGCGGGAGGAGAATGGTCTGAAGTACATC |
| | GAGGAGGCCATTGAGAAACTAAGCAAGCGGCACCAGTACCACATCCGTGCCTATGATC |
| | CCAAGGGAGGCCTGGACAATGCCCGACGTCTAACTGGATTCCATGAAACCTCCAACAT |
| | CAACGACTTTTCTGCTGGTGTAGCCAATCGTAGCGCCAGCATACGCATTCCCCGGACT |
| | GTTGGCCAGGAGAAGAAGGGTTACTTTGAAGATCGTCGCCCCTCTGCCAACTGCGACC |
| | CCTTTTCGGTGACAGAAGCCCTCATCCGCACGTGTCTTCTCAATGAAACCGGCGATGA |
| | GCCCTTCCAGTACAAAAATTAAGTGGACTAGACCTCCAGCTGTTGAGCCCCTCCTAGT |
| | TCTTCATCCCACTCCAACTCTTCCCCCTCTCCCAGTTGTCCCGATTGTAACTCAAAGG |
| | GTGGAATATCAAGGTCGTTTTTTTCATTCCATGTGCCCAGTTAATCTTGCTTTCTTTG |
| | TTTGGCTGGGATAGAGGGGTCAAGTTATTAATTTCTTCACACCTACCCTCCTTTTTTT |
| | CCCTATCACTGAAGCTTTTTAGTGCATTAGTGGGGAGGAGGGTGGGGAGACATAACCA |
| | CTGCTTCCATTTAATGGGGTGCACCTGTCCAATAGGCGTAGCTATCCGGACACAGCAC |
| | GTTTGCAGAAGGGGGACTCTTCTTCCAGGTAGCTGAAAGGGGAAGACCTGACGTACTC |
| | TGGTTAGGTTAGGACTTGCCCTCGTGGTGGAAACTTTTCTTAAAAAGTTATAACCAAC |
| | TTTTCTATTAAAAGTGGGAATTAGGAGAGAAGGTAGGGGTTGGGAATCAGAGAGAATG |
| | GCTTTGGTCTCTTGCTTGTGGGACTAGCCTGGCTTGGGACTAAATGCCCTGCTCTGAA |
| | CACGAAGCTTAGTATAAACTGATGGATATCCCTACCTTGAAAGAAGAAAAGGTTCTTA |
| | CTGCTTGGTCCTTGATTTATCACACAAAGCAGAATAGTATTTTTATATTTAAATGTAA |
| | AGACAAAAAACTATATGTATGGTTTTGTGGATTATGTGTGTTTTGCTAAAGGAAAAAA |
| | CCATCCAGGTCACGGGGCACCAAATTTGAGACAAATAGTCGGATTAGAAATAAAGCAT |
| | CTCATTTTGAGTAGAGAGCAAGGGAAGTGGTTCTTAGATGGTGATCTGGGATTAGGCC |
| | CTCAAGACCCTTTTGGGTTTCTGCCCTGCCCACCCTCTGGAGAAGGTGGGCACTGGAT |
| | TAGTTAACAGACGACACGTTACTAGCAGTCACTTGATCTCCGTGGCTTTGGTTTAAAA |
| | GACACACTTGTCCACATAGGTTTAGAGATAAGAGTTGGCTGGTCAACTTGAGCATGTT |
| | ACTGACAGAGGGGGTATTGGGGTTATTTTCTGGTAGGAATAGCATGTCACTAAAGCAG |
| | GCCTTTTGATATTAAATTTTTTAAAAAGCAAAATTATAGAAGTTTAGATTTTAATCAA |
| | ATTTGTAGGGTTTCTAGGTAATTTTTACAGAATTGCTTGTTTGCTTCAACTGTCTCCT |
| | ACCTCTGCTCTTGGAGGAGATGGGGACAGGGCTGGAGTCAAAACACTTGTAATTTTGT |
| | ATCTTGATGTCTTTGTTAAGACTGCTGAAGAATTATTTTTTTCTTTTATAATAAGGAA |
| | TAAACCCCACCTTTATTCCTTCATTTCATCTACCATTTTCTGGTTCTTGTGTTGGCTG |
| | TGGCAGGCCAGCTGTGGTTTTCTTTTGCCATGACAACTTCTAATTGCCATGTACAGTA |
| | TGTTCAAAGTCAAATAACTCCTCATTGTAAACAAACTGTGTAACTGCCCAAAGCAGCA |
| | CTTATAATCAGCCTAACATAAGAAAAAAAAAAAAAAAAAAAAAAAAAA |
| | ORF Start: ATG at 119 ORF Stop: TAA at 1238 |
| | SEQ ID NO: 76 373 aa MW at 42064.0kD |
| NOV3d, | MTTSASSHLNKGIKQVYMSLPQGEKVQAMYIWIDGTGEGLRCKTRTLDSEPKCVEELP |
| CG102822-04 | |
| Protein Sequence | EWNFDGSSTLQSEGSNSDMYLVPAAMFRDPFRKDPNKLVLCEVFKYNRRPAETNLRHT |
| | CKRIMDMVSNQHPWFCMEQEYTLMGTDGHPFGWPSNGFPGPQGPYYCGVGADRAYGRD |
| | IVEAHYRACLYAGVKIAGTNAEVMPAQWEFQIGPCEGISMGDHLWVARFILHRVCEDF |
| | GVIATFDPKPIPGNNNGAGCHTNFSTKAMREENGLKYIEEAIEKLSKRHQYHIRAYDP |
| | KGGLDNARRLTGFHETSNINDFSAGVANRSASIRIPRTVGQEKKGYFEDRRPSANCDP |
| | FSVTEALIRTCLLNETGDEPFQYKN |

Sequence comparison of the above protein sequences yields the following sequence relationships shown in Table 3B.

TABLE 3B
Comparison of NOV3a against NOV3b through NOV3d.

| Protein | NOV3a Residues/ | Identities/Similarities |
|---|---|---|
| Sequence | Match Residues | for the Matched Region |
| NOV3b | 15 . . . 387 | 369/373 (98%) |
| | 1 . . . 373 | 371/373 (98%) |
| NOV3c | 15 . . . 387 | 370/373 (99%) |
| | 1 . . . 373 | 372/373 (99%) |
| NOV3d | 15 . . . 387 | 370/373 (99%) |
| | 1 . . . 373 | 372/373 (99%) |

Further analysis of the NOV3a protein yielded the following properties shown in Table 3C.

TABLE 3C
Protein Sequence Properties NOV3a

| | |
|---|---|
| PSort | 0.5025 probability located in mitochondrial |
| analysis: | matrix space; 0.4633 probability located in |
| | microbody (peroxisome); 0.2227 probability |
| | located in mitochondrial inner membrane; |
| | 0.2227 probability located in mitochondrial |
| | intermembrane space |
| SignalP | No Known Signal Sequence Predicted |
| analysis: | |

A search of the NOV3a protein against the Geneseq database, a proprietary database that contains sequences published in patents and patent publication, yielded several homologous proteins shown in Table 3D.

TABLE 3D
Geneseq Results for NOV3a

| | | | Identities/ | |
|---|---|---|---|---|
| | | | Similarities for | |
| Geneseq | Protein/Organism/Length | NOV3a Residues/ | the Matched | Expect |
| Identifier | [Patent #, Date] | Match Residues | Region | Value |
| AAP70501 | Chinese hamster glutamine | 15 . . . 387 | 347/373 (93%) | 0.0 |
| | synthetase gene product - Cricetulus | 1 . . . 373 | 361/373 (96%) | |
| | griseus, 373 aa. [WO8704462-A, 30 | | | |
| | JUL. 1987] | | | |
| ABG08130 | Novel human diagnostic protein | 15 . . . 333 | 304/327 (92%) | 0.0 |
| | #8121 - Homo sapiens, 338 aa. | 1 . . . 320 | 305/327 (92%) | |
| | [WO200175067-A2, 11 OCT. 2001] | | | |
| ABB58458 | Drosophila melanogaster | 18 . . . 377 | 235/361 (65%) | e−150 |
| | polypeptide SEQ ID NO 2166 - | 9 . . . 369 | 292/361 (80%) | |
| | Drosophila melanogaster, 369 aa. | | | |
| | [WO200171042-A2, 27 SEP. 2001] | | | |
| ABB65740 | Drosophila melanogaster | 15 . . . 377 | 219/365 (60%) | e−132 |
| | polypeptide SEQ ID NO 24012 - | 36 . . . 399 | 271/365 (74%) | |
| | Drosophila melanogaster, 399 aa. | | | |
| | [WO200171042-A2, 27 SEP. 2001] | | | |

In a BLAST search of public sequence datbases, the NOV3a protein was found to have homology to the proteins shown in the BLASTP data in Table 3E.

TABLE 3E
Public BLASTP Results for NOV3a

| | | | Identities/ | |
|---|---|---|---|---|
| Protein | | | Similarities for | |
| Accession | | NOV3a Residues/ | the Matched | Expect |
| Number | Protein/Organism/Length | Match Residues | Portion | Value |
| AJHUQ | glutamate--ammonia ligase (EC | 15 . . . 387 | 372/373 (99%) | 0.0 |
| | 6.3.1.2) - human, 373 aa. | 1 . . . 373 | 373/373 (99%) | |
| P15104 | Glutamine synthetase (EC 6.3.1.2) | 15 . . . 387 | 370/373 (99%) | 0.0 |
| | (Glutamate--ammonia ligase) - | 1 . . . 373 | 372/373 (99%) | |
| | Homo sapiens (Human), 373 aa. | | | |
| AAH31964 | Similar to glutamine synthetase - | 15 . . . 387 | 368/373 (98%) | 0.0 |
| | Homo sapiens (Human), 373 aa. | 1 . . . 373 | 370/373 (98%) | |
| P46410 | Glutamine synthetase (EC 6.3.1.2) | 15 . . . 387 | 357/373 (95%) | 0.0 |
| | (Glutamate--ammonia ligase) - Sus | 1 . . . 373 | 364/373 (96%) | |
| | scrofa (Pig), 373 aa. | | | |
| Q91VC6 | Glutamine synthetase (EC 6.3.1.2) | 15 . . . 387 | 350/373 (93%) | 0.0 |
| | (Hypothetical 42.1 kDa protein) - | 1 . . . 373 | 362/373 (96%) | |
| | Mus musculus (Mouse), 373 aa. | | | |

PFam analysis predicts that the NOV3a protein contains the domains shown in the Table 3F.

TABLE 3F
Domain Analysis of NOV3a

| | | Identities/ | |
|---|---|---|---|
| | | Similarities for | |
| Pfam | NOV3a | the Matched | Expect |
| Domain | Match Region | Region | Value |
| gln-synt | 38 . . . 366 | 133/375 (35%) | 3e−198 |
| | | 298/375 (79%) | |

### Example 4

The NOV4 clone was analyzed, and the nucleotide and encoded polypeptide sequences are shown in Table 4A.

TABLE 4A
NOV4 Sequence Analysis

| | SEQ ID NO:77 | 1888 bp | |
|---|---|---|---|
| NOV4a, | AGCAGCCGGATGCCCGGGCCCACTGGGCGGGCCAGTGGCCGCTTGCGGGATGAGCAGA | | |
| CG103241-01 | | | |
| DNA Sequence | CTGCTGGGGGGGACGCTGGAGCGCGTCTGCAAGGCTGTGCTCCTTCTCTGCCTGCTGC | | |
| | ACTTCCTCGTGGCCGTCATCCTCTACTTTGACGTCTACGCCCAGCACCTGGCCTTCTT | | |
| | CAGCCGCTTCAGTGCCCGAGGCCCTGCCCATGCCCTCCACCCAGCTGCTAGCAGCAGC | | |
| | AGCAGCAGCAGCAACTGCTCCCGGCCCAACGCCACCGCCTCTAGCTCCGGGCTCCCTG | | |
| | AGGTCCCCAGTGCCCTGCCCGGTCCCACGGCTCCCACGCTGCCACCCTGTCCTGACAC | | |
| | CTCCCCGCCTGGTCTTGTGGGCAGACTGCTGATCGAGTTCACCTCACCCATGCCCCTG | | |
| | GAGCGGGTGCAGAGGGAGAACCCAGGCGTGCTCATGGGCGGCCGATACACATCGCCCG | | |
| | ACTGCACCCCAGCCCAGACGGTGGCGGTCATCATCCCCTTTAGACACCGGGAACACCA | | |
| | CCTGCGCTACTGGCTCCACTATCTACACCCCATCTTGAGGCCGCAGCGGCTGCGCTAC | | |
| | TGCGTCTATGTCATCAACCAGCATGGTGAGGACACCTTCAACCGGGCCAAGCTGCTTA | | |
| | ACGTGGGCTTCCTAGAGGCGCTGAAGGAGGATGCCGCCTATGACTGCTTCATCTTCAG | | |
| | CGATGTGGACCTGGTCCCCATGGATGACCGCAACCTATACCGCTGCGGCGACCAACCC | | |
| | CGCCACTTTGCCATTGCCATGGACAAGTTTGGCTTCCGGCTTCCCTATGCTGGCTACT | | |
| | TTGGAGGTGTGTCAGGCCTGAGTAAGGCTCAGTTTCTGAGAATCAATGGCTTCCCCAA | | |
| | TGAGTACTGGGGCTGGGGTGGCGAGGATGATGACATCTTCAACCGGATCTCCCTGACT | | |
| | GGGATGAAGATCTCACGCCCAGACATCCGAATTGGCCGCTACCGCATGATCAAGCACG | | |
| | ACCGCGACAACGATAACGAACCTAACCCTCAGAGGTTTACCAAGATTCAAAACACGAA | | |
| | GCTGACCATGAAGCGGGACGGCATTGGGTCAGTGCGGTACCAGGTCTTGGAGGTGTCT | | |
| | CGCCAACCACTCTTCACCAATATCACAGTGGACATTGGGCGGCCTCCGTCGTGGCCCC | | |
| | CTCGGGGCTGACACTAATGGACAGAGGCTCTCGGTGCCGAAGATTGCCTGCCAGAGGA | | |
| | CTGACCACAGCCTGGCTGGCAGCTGCTCTGTGGAGGACCTCCAGGACTGAGACTGGGC | | |
| | TCTGTTTTCCAAGGGTCTTCACTAGGCCCCCTAGCTATACCTGGAAGTTTCAGAACCC | | |
| | ACTTTGGGGGCCTCTCCGTCGGCAGGCTCTTCAAGTGTGGCCCTCTTTGGAGTCAACC | | |
| | CTCCTTCCCGACCCCCTCCCCCTAGCCCACCCCCAGTCACTGTCAGGGTCGGCCAGCC | | |
| | CCTGCACTGCCTCGCAGAGTGGCCTGGGCTAGGTCACTCCACCTCTCTGTGCCTCAGT | | |
| | TTCCCCCCCTTGAGTCCCCTTAGGGCCTGGAAGGGTGGGACGTATGTCTAGGGGGCAA | | |
| | GTGTCTCTTCCAGGGGGAATTCTCAGCTCTTGGGAACCCCCTTGCTCCCAGGGGAGGG | | |
| | GAAACCTTTTTCATTCAACATTGTACGGGGCAAGCTTTGGTGCGCCCCCTGCTGAGGA | | |
| | GCGAGCCCAGGAGGGGACCAGAGGGGATGCTCTGTCCCTGCCTGGGATCTTGGGGTTG | | |
| | GCCTTTGCATGGGAGGCAGGTGGGGCTTGGATCAGTAAGTCTGGTTCCCGCCTCCCTG | | |
| | TCTGAGAGAGGAGGCAGGANCCCAGGGCCGGCTTGTGTTTGTACATTGCACAGAAACT | | |
| | TGTGTGGCTGCTTTACTAAAAAACGTGAATGG | | |
| | ORF Start: ATG at 50 | | ORF Stop: TGA at 1169 |
| | SEQ ID NO:78 | 373 aa | MW at 42072.7 kD |
| NOV4a, | MSRLLGGTLERVCKAVLLLCLLHFLVAVILYFDVYAQHLAFFSRFSARGPAHALHPAA | | |
| CG103241-01 | | | |
| Protein Sequence | SSSSSSSNCSRPNATASSSGLPEVPSALPGPTAPTLPPCPDTSPPGLVGRLLIEFTSP | | |
| | MPLERVQRENPGVLMGGRYTSPDCTPAQTVAVIIPFRHREHHLRYWLHYLHPILRRQR | | |
| | LRYCVYVINQHGEDTFNRAKLLNVGFLEALKEDAAYDCFIFSDVDLVPMDDRNLYRCG | | |
| | DQPRHFAIAMDKFGFRLPYAGYFGGVSGLSKAQFLRINGFPNEYWGWGGEDDDIFNRI | | |
| | SLTGMKISRPDIRIGRYRMIKHDRDNDNEPNPQRFTKIQNTKLTMKRDGIGSVRYQVL | | |
| | EVSRQPLFTNITVDIGRPPSWPPRG | | |
| | SEQ ID NO:79 | 1783 bp | |
| NOV4b, | AGCAGCCGGATGCCCGGGCCCACTGGGCGGGCCAGTGGCCGCTTGCGGGATGAGCAGA | | |
| CG103241-02 | | | |
| DNA Sequence | CTGCTGGGGGGGACGCTGGAGCGCGTCTGCAAGGCTGTGCTCCTTCTCTGCCTGCTGC | | |
| | ACTTCCTCGTGGCCGTCATCCTCTACTTTGACGTCTACGCCCAGCACCTGGCCTTCTT | | |
| | CAGCCGCTTCAGTGCCCGAGGCCCTGCCCATGCCCTCCACCCAGCTGCTAGCAGCAGC | | |
| | AGCAGCAGCAGCAACTGCTCCCGGCCCAACGCCACCGCCTCTAGCTCCGGGCTCCCTG | | |
| | AGGTCCCCAGTGCCCTGCCCGGTCCCACGGCTCCCACGCTGCCACCCTGTCCTGACAC | | |
| | CTCCCCGCCTGGTCTTGTGGGCAGACTGCTGATCGAGTTCACCTCACCCATGCCCCTG | | |
| | GAGCGGGTGCAGAGGGAGAACCCAGGCGTGCTCATGGGCGGCCGATACACATCGCCCG | | |
| | ACTGCACCCCAGCCCAGACGGTGGCGGTCATCATCCCCTTTAGACACCGGGAACACCA | | |
| | CCTGCGCTACTGGCTCCACTATCTACACCCCATCTTGAGGCGGCAGCGGCTGCGCTAC | | |
| | TGCGTCTATGTCATCAACCAGCATGGTGAGGACACCTTCAACCGGGCCAAGCTGCTTA | | |
| | ACGTGGGCTTCCTAGAGGCGCTGAAGGAGGATGCCGCCTATGACTGCTTCATCTTCGG | | |
| | CGATGTGGACCTGGTCCCCATGGATGACCGCAACCTATACCGCTGCGGCGACCAACCC | | |
| | CGCCACTTTGCCATTGCCATGGACAAGTTTGGCTTCCGGCTTCCCTATGCTGGCTACT | | |
| | TTGGAGGTGTGTCAGGCCTGAGTAAGGCTCAGTTTCTGAGAATCAATGGCTTCCCCAA | | |
| | TGAGTACTGGGGCTGGGGTGGCGAGGATGATGACATCTTCAACCGGTTTACCAAGATT | | |
| | CAAAACACGAAGCTGACCATGAAGCGGGACGACATTGGGTCAGTGCGGTACCAGGTCT | | |
| | TGGAGGTGTCTCGGCAACCACTCTTCACCAATATCACAGTGGACATTGGGCGGCCTCC | | |
| | GTCGTGGCCCCCTCGGGGCTGACACTAATGGACAGAGGCTCTCGGTGCCGAACATTGC | | |
| | CTGCCAGAGGACTGACCACAGCCTGGCTGGCAGCTGCTCTGTGGAGGACCTCCAGGAC | | |
| | TGAGACTGGGCTCTGTTTTCCAAGGGTCTTCACTAGGCCCCCTAGCTATACCTGGAAG | | |
| | TTTCAGAACCCACTTTGGGGGCCTCTCCGTGGGCAGGCTCTTCAAGTGTGGCCCTCTT | | |
| | TGGAGTCAACCCTCCTTCCCGACCCCCTCCCCCTAGCCCAGCCCCAGTCACTGTCAGG | | |
| | GTCGGCCAGCCCCTGCACTGCCTCGCAGAGTGGCCTGGGCTAGGTCACTCCACCTCTC | | |
| | TGTGCCTCAGTTTCCCCCCCTTGAGTCCCCTTAGGGCCTGGAAGGGTGGGAGGTATGT | | |
| | CTAGGGGGCAAGTGTCTCTTCCAGGGGGAATTCTCAGCTCTTGGGAACCCCCTTGCTC | | |
| | CCAGGGGAGGGGAAACCTTTTTCATTCAACATTGTAGGGGGCAAGCTTTGGTGCGCCC | | |
| | CCTGCTGAGGAGCGAGCCCAGGAGGGGACCAGAGGGGATGCTGTGTCGCTGCCTGGGA | | |
| | TCTTGGGGTTGGCCTTTGCATGGCAGGCAGGTGGGGCTTGGATCAGTAAGTCTGGTTC | | |
| | CCGCCTCCCTGTCTGAGAGAGGAGGCAGGAACCCAGGGCCGGCTTGTGTTTGTACATT | | |
| | GCACAGAAACTTGTGTGGGTGCTTTAGTAAAAAACGTGAATGG | | |
| | ORF Start: ATG at 50 | | ORF Stop: TGA at 1064 |
| | SEQ ID NO:80 | 338 aa | MW at 37925.0 kD |
| NOV4b, | MSRLLGGTLERVCKAVLLLCLLHFLVAVILYFDVYAQHLAFFSRFSARGPAHALHPAA | | |
| CG103241-02 | | | |
| Protein Sequence | SSSSSSSNCSRPNATASSSGLPEVPSALPGPTAPTLPPCPDTSPPGLVGRLLIEFTSP | | |
| | MPLERVQRENPGVLMGGRYTSPDCTPAQTVAVILPFRHREHHLRYWLHYLHPILRRQR | | |
| | LRYCVYVINQHGEDTFNRAKLLNVGFLEALKEDAAYDCFIFGDVDLVPMDDRNLYRCG | | |
| | DQPRHFAIANDKFGFRLPYAGYFGGVSGLSKAQFLRINGFPNEYWGWGGEDDDIFNRF | | |
| | TKIQNTKLTMKRDDIGSVRYQVLEVSRQPLFTNITVDIGRPPSWPPRG | | |
| | SEQ ID NO:81 | 1119 bp | |
| NOV4c, | ATGAGCAGACTGCTGGGGGGGACGCTGGAGCGCGTCTGCAAGGCTGTGCTCCTTCTCT | | |
| CG103241-03 | | | |
| DNA Sequence | GCCTGCTGCACTTCCTCGTGGCCGTCATCCTCTACTTTGACGTCTACGCCCAGCACCT | | |
| | GGCCTTCTTCAGCCGCTTCAGTGCCCGAGGCCCTGCCCATGCCCTCCACCCAGCTGCT | | |
| | AGCAGCAGCAGCAGCAGCAGCAACTGCTCCCGGCCCAACGCCACCGCCTCTAGCTCCG | | |
| | GGCTCCCTGAGGTCCCCAGTGCCCTGCCCGGTCCCACGGCTCCCACGCTGCCACCCTG | | |
| | TCCTGACTCGCCACCTCGTCTTGTGGGCAGACTGCTGATCGAGTTCACCTCACCCATG | | |
| | CCCCTGGAGCGGGTGCACAGGGAGAACCCACGCGTGCTCATGGGCGGCCGATACACAC | | |
| | CGCCCGACTGCACCCCAGCCCAGACGGTGGCGGTCATCATCCCCTTTAGACACCGGGA | | |
| | ACACCACCTGCGCTACTGGCTCCACTATCTACACCCCATCTTGAGGCGGCAGCGGCTG | | |
| | CGCTACGGCGTCTATGTCATCAACCAGCATGGTGAGGACACCTTCAACCGGGCCAAGC | | |
| | TGCTTAACGTGGGCTTCCTAGAGGCGCTGAAGGAGGATGCCGCCTATGACTGCTTCAT | | |
| | CTTCAGCGATGTGGACCTGGTCCCCATGGATGACCGCAACCTATACCGCTGCGGCGAC | | |
| | CAACCCCGCCACTTTGCCATTGCCATGGACAAGTTTGGCTTCCGGCTTCCCTATGCTG | | |
| | GCTACTTTGGAGGTGTGTCAGGCCTGAGTAAGGCTCAGTTTCTGAGAATCAATGGCTT | | |
| | CCCCAATGAGTACTGGGGCTGGGGTGGCGAGGATGATGACATCTTCAACCGGATCTCC | | |
| | CTGACTGGGATGAAGATCTCACGCCCAGACATCCGAATTGGCCGCTACCGCATGATCA | | |
| | AGCACGACCGCGACAAGCATAACGAACCTAACCCTCAGAGGTTTACCAAGATTCAAAA | | |
| | CACGAAGCTGACCATGAAGCGGGACGGCATTGGGTCAGTGCGGTACCAGGTCTTGGAG | | |
| | GTGTCTCGGCAACCACTCTTCACCAATATCACAGTGGACATTGGGCGGCCTCCGTCGT | | |
| | GGCCCCCTCGGGGCTGA | | |
| | ORF Start: ATG at 1 | | ORF Stop: TGA at 1117 |
| | SEQ ID NO:82 | 372 aa | MW at 41980.7 kD |

| | |
|---|---|
| NOV4c, | MSRLLGGTLERVCKAVLLLCLLHFLVAVILYFDVYAQHLAFFSRFSARGPAHALHPAA |
| CG103241-03 | |
| Protein Sequence | SSSSSSSNCSRPNATASSSGLPEVPSALPGPTAPTLPPCPDSPPGLVGRLLIEFTSPM |
| | PLERVHRENPGVLMGGRYTPPDCTPAQTVAVIIPFRHREHHLRYWLHYLHPILRRQRL |
| | RYCVYVINQHGEDTFNRAKLLNVGFLEALKEDAAYDCFIFSDVDLVPMDDRNLYRCGD |
| | QPRHFAIAMDKFGFRLPYAGYFGGVSGLSKAQFLRINGFPNEYWGWGGEDDDIFNRIS |
| | LTGMKISRPDIRIGRYRMIKHDRDKHNEPNPQRFTKIQNTKLTMKRDGIGSVRYQVLE |
| | VSRQPLFTNITVDIGRPPSWPPRG |

Sequence comparison of the above protein sequences yields the following sequence relationships shown in Table 4B.

TABLE 4B
Comparison of NOV4a against NOV4b and NOV4c.

| | | Identities/ |
|---|---|---|
| | | Similarities for |
| Protein | NOV4a Residues/ | the Matched |
| Sequence | Match Residues | Region |
| NOV4b | 1 . . . 373 | 336/373 (90%) |
| | 1 . . . 338 | 336/373 (90%) |
| NOV4c | 1 . . . 373 | 367/373 (98%) |
| | 1 . . . 372 | 367/373 (98%) |

Further analysis of the NOV4a protein yielded the following properties shown in Table 4C.

TABLE 4C
Protein Sequence Properties NOV4a

| | |
|---|---|
| PSort | 0.8650 probability located in lysosome |
| analysis: | (lumen); 0.8200 probability located in |
| | outside; 0.2030 probability located in |
| | microbody (peroxisome); 0.1000 probability |
| | located in endoplasmic reticulum (membrane) |
| SignalP | Cleavage site between residues 37 and 38 |
| analysis: | |

A search of the NOV4a protein against the Geneseq database, a proprietary database that contains sequences published in patents and patent publication, yielded several homologous proteins shown in Table 4D.

TABLE 4D
Geneseq Results for NOV4a

| | | | Identities/ | |
|---|---|---|---|---|
| | | | Similarities for | |
| Geneseq | Protein/Organism/Length | NOV4a Residues/ | the Matched | Expect |
| Identifier | [Patent #, Date] | Match Residues | Region | Value |
| AAM93215 | Human polypeptide, SEQ ID NO: | 117 . . . 373 | 253/257 (98%) | e−153 |
| | 2618 - Homo sapiens, 257 aa. | 1 . . . 257 | 253/257 (98%) | |
| | [EP1130094-A2, 05 SEP. 2001] | | | |
| AAY17862 | Human beta-1,4-galactose | 6 . . . 366 | 204/384 (53%) | e−109 |
| | transferase - Homo sapiens, 398 aa. | 16 . . . 397 | 247/384 (64%) | |
| | [JP11137247-A, 25 MAY 1999] | | | |
| AAB03647 | Beta 1,4 galactose transferase | 6 . . . 366 | 204/384 (53%) | e−109 |
| | protein sequence - Homo sapiens, | 3 . . . 384 | 247/384 (64%) | |
| | 385 aa. [WO200034490-A1, 15 | | | |
| | JUN. 2000] | | | |
| AAR28838 | HeLa cell galactosyltransferase | 6 . . . 366 | 204/384 (53%) | e−109 |
| | enzyme - Homo sapiens, 398 aa. | 16 . . . 397 | 247/384 (64%) | |
| | [GB2256197-A, 02 DEC. 1992] | | | |
| AAR55706 | Galactosyltransferase - Homo | 6 . . . 366 | 204/384 (53%) | e−109 |
| | sapiens, 398 aa. [WO9412646-A, | 16 . . . 391 | 247/384 (64%) | |
| | 09 JUN. 1994] | | | |

In a BLAST search of public sequence datbases, the NOV4a protein was found to have homology to the proteins shown in the BLASTP data in Table 4E.

TABLE 4E
Public BLASTP Results for NOV4a

| | | | Identities/ | |
|---|---|---|---|---|
| Protein | | | Similarities for | |
| Accession | | NOV4a Residues/ | the Matched | Expect |
| Number | Protein/Organism/Length | Match Residues | Portion | Value |
| O60909 | Beta-1,4-galactosyltransferase 2 (EC | 1 . . . 373 | 368/373 (98%) | 0.0 |
| | 2.4.1.-) (Beta-1,4-GalTase 2) | 1 . . . 372 | 368/373 (98%) | |
| | (Beta4Gal-T2) (b4Gal-T2) (UDP- | | | |
| | galactose: beta-N-acetylglucosamine | | | |
| | beta-1,4-galactosyltransferase 2) | | | |
| | (UDP-Gal: beta-GlcNAc beta-1,4- | | | |
| | galactosyltransferase 2) [Includes: | | | |
| | Lactose synthase A protein (EC | | | |
| | 2.4.1.22); N-acetyllactosamine synthase | | | |
| | (EC 2.4.1.90) (Nal synthetase); Beta-N- | | | |
| | acetylglucosaminyl-glycopeptidebeta- | | | |
| | 1,4-galactosyltransferase (EC | | | |
| | 2.4.1.38); Beta-N-acetylglucosaminyl- | | | |
| | glycolipid beta-1,4- | | | |
| | galactosyltransferase (EC 2.4.1.-)] - | | | |
| | Homo sapiens (Human), 372 aa. | | | |
| Q9Z2Y2 | Beta-1,4-galactosyltransferase II - Mus | 1 . . . 373 | 338/373 (90%) | 0.0 |
| | musculus (Mouse), 369 aa. | 1 . . . 369 | 354/373 (94%) | |
| Q92073 | Beta-1,4-galactosyltransferase (EC | 4 . . . 373 | 278/378 (73%) | e−164 |
| | 2.4.1.38) - Gallus gallus (Chicken), 373 | 5 . . . 373 | 317/378 (83%) | |
| | aa. | | | |
| T46511 | hypothetical protein | 150 . . . 373 | 221/224 (98%) | e−132 |
| | DKFZp586M2424.1 - human, 224 aa | 1 . . . 224 | 221/224 (98%) | |
| | (fragment). | | | |
| CAA01685 | GALACTOSYLTRANSFERASE - | 6 . . . 366 | 204/384 (53%) | e−108 |
| | Homo sapiens (Human), 398 aa. | 16 . . . 397 | 247/384 (64%) | |

PFam analysis predicts that the NOV4a protein contains the domains shown in the Table 4F.

TABLE 4F
Domain Analysis of NOV4a

| | | Identities/ | |
|---|---|---|---|
| | | Similarities for | |
| Pfam | NOV4a | the Matched | Expect |
| Domain | Match Region | Region | Value |
| Galactosyl_T_2 | 97 . . . 367 | 169/330 (51%) | 5.5e−190 |
| | | 268/330 (81%) | |

### Example 5

The NOV5 clone was analyzed, and the nucleotide and encoded polypeptide sequences are shown in Table 5A.

TABLE 5A
NOV5 Sequence Analysis

| | SEQ ID NO:83 | 4215 bp | |
|---|---|---|---|
| NOV5a, | CGATGGCATCGGTCAAGGTGGCCGTGAGGGTCCGGCCCATGAATCGCAGGGAAAAGGA | | |
| CG106249-01 | | | |
| DNA Sequence | CTTGGAGGCCAAGTTCATTATTCAGATGGAGAAAAGCAAAACGACAATCACAAACTTA | | |
| | AAGATACCAGAAGGAGGCACTGGGGACTCAGGAAGAGAACGGACCAAGACCTTCACCT | | |
| | ATGACTTTTCTTTTTATTCTCCTGATACAAAAACTACAGACTACGTTTCACAAGAAAT | | |
| | GGTTTTCAAAACCCTCCGCACAGATGTCCTGAATTCTGCATTTGAAGTTTATAATGCT | | |
| | TGTGTCTTTGCATATGGGCAAACTGGATCTGGAAAGTCCTACGCTATGATGGGAAATT | | |
| | CTGGAGATTCTGGCTTAATACCTCGGATCTGTCAAGGACTCTCCATTCGGATTAATGA | | |
| | AACCACCAGATCGGATGAAGCTTCTTTCCGAACTGAAGTCAGCTCCTTAAAAATTTAT | | |
| | AACGAACGTGTGAGAGATCTACTTCCGCGGAAGTCATCTAAAACCTTCAATTTGAGAG | | |
| | TCCGTGAGCATCCCAAAGAAGGCCCTTATGTTGAGGATTTATCCAAACATTTAGTACA | | |
| | GAATTATGGTGACGTAGAAGAACTTATGGATGCGGGCAATATCAACCGGACCACCGCA | | |
| | GCGACTGGGATGAACGACGTCAGTAGCAGGTCTCATGCCATCTTCACCATCAAGTTCA | | |
| | CTCAGGCTAAATTTGATTCTGAAATGCCATGTGAAACCGTCAGTAAGATCCACTTGGT | | |
| | TGATCTTGCCGGAAGTGAGCGTGCAGATGCCACCGGAGCCACCGGGGTTAGGCTAAAG | | |
| | GAAGGGGGAAATATTAACAAGTCCCTTGTGACTCTGGGGAACGTCATTTCTGCCTTAG | | |
| | CTGATTTATCTCAGGATGCTGCAAATACTCTTGCAAAGAAGAAGCAAGTTTTCGTGCC | | |
| | TTACAGGGATTCTGTGTTGACTTGGTTGTTAAAAGATAGCCTTGGAGGAAACTCTAAA | | |
| | ACTATCATGATTGCCACCATTTCACCTGCTGATGTCAATTATGGAGAAACCCTAAGTA | | |
| | CTCTTCGCTATGCAAATAGAGCCAAAAACATCATCAACAAGCCTACCATTAATGAGGA | | |
| | TGCCAACGTCAAACTTATCCGTGAGCTGCGAGCTGAAATAGCCAGACTGAAAACGCTG | | |
| | CTTGCTCAAGGGAATCAGATTGCCCTCTTAGACTCCCCCACAGCTTTAAGTATGGAGG | | |
| | AAAAACTTCAGCAGAATGAAGCAAGAGTTCAAGAATTGACCAAGGAATGGACAAATAA | | |
| | GTGGAATGAAACCCAAAATATTTTGAAAOAACAAACTCTAGCCCTCAGGAAAGAAGGG | | |
| | ATTGGAGTTGTTTTGGATTCTGAACTGCCTCATTTGATTGGCATCGATGATGACCTTT | | |
| | TGAGTACTGGAATCATCTTATATCATTTAAAGGAAGGTCAGACATACGTTGGTAGAGA | | |
| | CGATGCTTCCACGGAGCAAGATATTGTTCTTCATGGCCTTGACTTGGACAGTGAGCAT | | |
| | TGCATCTTTGAAAATATCGGGGGGACAGTGACTCTGATACCCCTGAGTGGGTCCCAGT | | |
| | GCTCTGTGAATGGTGTTCAGATCGTGGAGGCCACACATCTAAATCAAGGTGCTGTGAT | | |
| | TCTCTTGGGAAGAACCAATATGTTTCGCTTTAACCATCCAAAGGAAGCCGCCAAGCTC | | |
| | AGGGAGAAGAGGAAGAGTGGCCTTCTGTCCTCCTTCAGCTTGTCCATGACCGACCTCT | | |
| | CGAAGTCCCGTGAGAACCTGTCTGCAGTCATGTTGTATAACCCCGGACTTGAGTTTGA | | |
| | GAGGCAACAGCGTGAAGAACTTGAAAAATTAGAAAGTAAAAGGAAACTCATTGAGGAA | | |
| | ATGGAGGAAAAGCAGAAATCGGACAAGGCTGAACTGGAGCGGATGCAGCAGGAGGTGG | | |
| | AGACCCAGCGCAAGGAGACAGAAATCGTGCAGCTCCAGATTCGCAAGCAGGAGGAGAG | | |
| | CCTCAAACGCCGCAGCTTCCACATCGAGAACAAGCTAAAGGATTTACTTGCGGAGAAG | | |
| | GAAAAATTTGAAGAGGAGAGGCTGAGGGAACACCAGGAAATCGAGCTGCAGAAGAAGA | | |
| | GACAAGAAGAAGAGACCTTTCTCCGCGTCCAAGAAGAACTCCAACGACTCAAAGAACT | | |
| | CAACAACAACGAGAAGGCTGAGAAGTTTCAGATATTTCAAGAACTGGACCAGCTCCAA | | |
| | AAGGAAAAAGATGAACAGTATGCCAAGCTTGAACTGGAAAAAAAGAGACTAGAGGAGC | | |
| | AGGAGAAGGAGCAGGTCATGCTCGTGGCCCATCTGGAAGAGCAGCTCCGAGAGAAGCA | | |
| | GGAGATGATCCAGCTCCTGCGGCGTGGGGAGGTACAGTGGGTGGAAGAGGAGAAGAGG | | |
| | GACCTGGAAGGCATTCGGGAATCCCTCCTGCGGGTGAAGGAGGCTCGTGCCGGAGGGG | | |
| | ATGAAGATGGCGAGGAGTTAGAAAAGGCTCAACTGCGTTTCTTCGAATTCAAGAGAAG | | |
| | GCAGCTTGTCAAGCTAGTGAACTTCGAGAAGGACCTGGTTCAGCAGAAAGACATCCTG | | |
| | AAAAAAGAAGTCCAAGAAGAACAGGAGATCCTAGAGTGTTTAAAATGTGAAcATGACA | | |
| | AAGAATCTAGATTGTTCGAAAAACATGATGAGACTGTCACAGATGTCACGGAAGTGCC | | |
| | TCAAGATTTCGAGAAAATAAAGCCAGTGGAGTACAGGCTGCAATATAAAGAACGCCAG | | |
| | CTACAGTACCTCCTGCAGAATCACTTGCCAACTCTGTTGGAAGAAAAGCAGAGAGCAT | | |
| | TTGAAATTCTTGACAGAGGCCCTCTCAGCTTAGACAACACTCTTTATCAAGTAGAAAA | | |
| | GGAAATGGAAGAAAAAGAAGAACAGCTTGCACAGTACCAGGCCAATGCAAACCAGCTG | | |
| | CAAAAGCTCCAAGCCACCTTTGAATTCACTGCCAACATTGCACGTCAGCAGGAAAAAG | | |
| | TGAGGAAAAAGGAAAAGGAGATTTTGGAGTCCAGAGAGAAGCAGCAGAGAGAGGCGCT | | |
| | GGAGCGGGCCCTGGCCAGGCTGCAGAGGACACATTCTGCGCTGCAGAGGCACTCCACC | | |
| | CTGGGCACGGAGATTGAAGAGCAGAGGCAGAAACTTGCCAGTGTGAACAGTGGCAGCA | | |
| | GAGAGCAGTCAGGGTTCCAGGCTAGCCTGGAGGCTGAGCAGCAAGCACTAGAGATGTA | | |
| | CCATGTAGAAAGGTTAGAATATGAAATCCAGCAGCTGAAACAGAAGATTTATGAGGTC | | |
| | GATGGTGTTCAAAAAGATCATCATGGGACCCTGGAAGGGAAGGTGGCTTCTTCCAGCT | | |
| | TGCCAGTCAGTGCTGAAAAATCACACCTGGTTCCCCTCATGGATGCCAGGAGGATCAA | | |
| | TGCTTACATTGAAGAAGAAGTCCAAAGACGCCTTCAGGATTTGCATCGTGTGATTAGT | | |
| | GAAGCCTGCAGTACATCTGCAGACACGATGAAGGATAATGAGAAACTTCACAATGGCA | | |
| | CCATTCAACGTAAACTAAAATATGAGCTGTGTCGTGACCTCCTGTGTGTCCTGATGCC | | |
| | AGAGCCTGATGCCGCTGCCTGCGCTAATCATCCCTTGCTCCAGCAACATCTGGTTCAG | | |
| | CTTTCTCTTGATTGGAAAACAGAAATCCCTGATTTAGTTTTGCCAAATGGAGTTCAGG | | |
| | TGTCATCCAAATTCCAGACTACCTTGGTTCACATGATTTACTTTCTTCATGGAAATAT | | |
| | GGAAGTCAATGTCCCTTCCCTGGCAGAAGTTCAGTTACTGCTCTACACAACAGTGAAA | | |
| | GTCATGGGTGACTCTGGCCATGACCAGTGCCAGTCGCTAGTCCTTCTGAACACCCACA | | |
| | TTGCACTGGTGAAGGAAGACTGTGTTTTTTATCCACGCATTCGATCTCGAAACATACC | | |
| | TCCTCCGGGTCCACAATTTGATGTGATCAAATGCCATGCTTTAAGTGAATTCAGGTGT | | |
| | GTTGTTGTTCCAGAAAAGAAAAATGTGTCAACAGTAGAACTAGTCTTCTTACAGAAAC | | |
| | TCAAACCTTCAGTGGGTTCCAGAAATAGTCCACCTGAGCACCTTCAGGAAGCCCCAAA | | |
| | TGTCCAGTTGTTCACCACCCCATTGTATCTTCAAGGCAGTCAGAATGTCGCACCTGAG | | |
| | GTCTGGAAACTTACTTTCAATTCTCAAGATGAGGCTCTTTGGCTAATCTCACATTTGA | | |
| | CAAGACTCTAAGGAGGAGACTTTTAAAGATGCACTACAT | | |
| | ORF Start: ATG at 3 | | ORF Stop: TAA at 4185 |
| | SEQ ID NO:84 | 1394 aa | MW at 160054.1 kD |
| NOV5a, | MASVKVAVRVRPMNRREKDLEAKFIIQMEKSKTTITNLKIPEGGTGDSGRERTKTFTY | | |
| CG106249-01 | | | |
| Protein Sequence | DFSFYSADTKTTDYVSQEMVFKTLRTDVLNSAFEVYNACVFAYGQTGSGKSYAMMGNS | | |
| | GDSGLIPRICEGLSIRINETTRSDEASFRTEVSSLKIYNERVRDLLRRKSSKTFNLRV | | |
| | REHPKECPYVEDLSKHLVQNYGDVEELMDACNINRTTAATGMNDVSSRSNAIFTIKFT | | |
| | QAKFDSEMPCETVSKIHLVDLAGSERADATGATGVRLKEGGNINKSLVTLGNVISALA | | |
| | DLSQDAANTLAKKKQVFVPYRDSVLTWLLKDSLOGNSKTIMIATISPADVNYGETLST | | |
| | LRYANRAKNIINKPTINEDANVKLIRELRAEIARLKTLLAQGNQIALLDSPTALSMEE | | |
| | KLQQNEARVQELTKEWTNKWNETQNTLKEQTLALRKEGIGVVLDSELPHLIGIDDDLL | | |
| | STGIILYHLKEGQTYVGRDDASTEQDIVLHGLDLESEHCIFENIGGTVTLIPLSGSQC | | |
| | SVNGVQIVEATHLNQGAVILLCRTNMFRFNHPKEAAKLREKRKSGLLSSFSLSMTDLS | | |
| | KSRENLSAVMLYNPGLEFERQQREELEKLESKRKLIEEMEEKQKSDKAELERMQQEVE | | |
| | TQRKETEIVQLQIRKQEESLKRRSFHIENKLKDLLAEKEKFEEERLREQQEIELQKKR | | |
| | QEEETFLRVQEELQRLKELNNNEKAEKFQIFQELDQLQKEKDEQYAKLELEKKRLEEQ | | |
| | EKEQVMLVAHLEEQLREKQEMIQLLRRGEVQWVEEEKRDLEGIRESLLRVKEARAGGD | | |
| | EDGEELEKAQLRFFEFKRRQLVKLVNLEKDLVQQKDILKKEVQEEQEILECLKCEHDK | | |
| | ESRLLEKHDESVTDVTEVPQDFEKTKPVEYRLQYKERQLQYLLQNHLPTLLEEKQRAF | | |
| | EILDRGPLSLDNTLYQVEKEMEEKEEQLAQYQANANQLQKLQATFEFTANIARQEEKV | | |
| | RKKEKEILESREKQQREALERALARLERRHSALQRHSTLGTEIEEQRQKLASVNSGSR | | |
| | EQSGFQASLEAEQEALEMYHVERLEYEIQQLKQKIYEVDGVQKDHHGTLEGKVASSSL | | |
| | PVSAEKSHLVPLMDARRINAYIEEEVQRRLQDLHRVISEGCSTSADTMKDNEKLHNGT | | |
| | IQRKLKYELCRDLLCVLMPEPDAAACANHPLLQQDLVQLSLDWKTEIPDLVLPNGVQV | | |
| | SSKFQTTLVDMIYFLHGNMEVNVPSLAEVQLLLYTTVKVMGDSGHDQCQSLVLLNTHI | | |
| | ALVKEDCVFYPRIRSRNIPPPGAQFDVIKCHALSEFRCVVVPEKKNVSTVELVFLQKL | | |
| | KPSVGSRNSPPEHLQEAPNVQLFTTPLYLQGSQNVAPEVWKLTFNSQDEALWLISHLT | | |
| | RL | | |
| | SEQ ID NO:85 | 4502 bp | |
| NOV5b, | CGGCACGAGGGGGATGAGCGATGGCATCGGTCAAGGTGGCCGTGAGGGTCCGGCCCAT | | |
| CG106249-02 | | | |
| DNA Sequence | GAATCGCAGGGAAAAGGACTTGGAGGCCAAGTTCATTATTCAGATGGAGAAAAGCAAA | | |
| | ACGACAATCACAAACTTAAACATACCAGAAGGAGGCACTGGGGACTCAGGAAGAGAAC | | |
| | GGACCAAGACCTTCACCTATGACTTTTCTTTTTATTCTGCTGATACAAAAAGCCCAGA | | |
| | TTACGTTTCACAAGAAATGGTTTTCAAAACCCTCGGCACAGATGTCGTGAAGTCTGCA | | |
| | TTTGAAGGTTATAATGCTTGTGTCTTTGCATATGGGCAAACTGGATCTGGAAAGTCAT | | |
| | ACACTATGATGGGAAATTCTGGAGATTCTGGCTTAATACCTCGGATCTGTGAAGGACT | | |
| | CTTCAGTCGGATAAATGAAACCACCAGATGGGATGAAGCTTCTTTTCGAACTGAAGTC | | |
| | AGCTACTTAGAAATTTATAACGAACGTGTGAGAGATCTACTTCGGCGGAAGTCATCTA | | |
| | AAACCTTCAATTTGAGAGTCCGTGAGCATCCCAAAGAAGGCCCTTATGTTGAGGATTT | | |
| | ATCCAAACATTTAGTACAGAATTATGGTGACGTAGAAGAACTTATGGATGCGGGCAAT | | |
| | ATCAACCGGACCACCGCAGCGACTGGGATGAACGACGTCAGTAGCAGGTCTCATGCCA | | |
| | TCTTCACCATCAAGTTCACTCAGGCTAAATTTGATTCTGAAATGCCATGTGAAACCGT | | |
| | CAGTAAGATCCACTTGGTTGATCTTGCCGGAAGTGAGCGTGCAGATGCCACCGGAGCC | | |
| | ACCGGGGTTAGGCTAAAGGAAGGGGGAAATATTAACAAGTCCCTTGTGACTCTGGGGA | | |
| | ACGTCATTTCTGCCTTAGCTGATTTATCTCAGGATGCTGCAAATACTCTTGCAAAGAA | | |
| | GAAGCAAGTTTTCGTGCCTTACAGGGATTCTGTGTTGACTTGGTTGTTAAAAGATAGC | | |
| | CTTGGAGGAAACTCTAAAACTATCATGATTGCCACCATTTCACCTGCTGATGTCAATT | | |
| | ATGGAGAAACCCTAAGTACTCTTCGCTATGCAAATAGAGCCAAAAACATCATCAACAA | | |
| | GCCTACCATTAATGAGGATGCCAACGTCAAACTTATCCGTGAGCTGCGAGCTGAAATA | | |
| | GCCAGACTGAAAACGCTGCTTGCTCAAGGGAATCAGATTGCCCTCTTAGACTCCCCCA | | |
| | CAGCTTTAAGTATGGAGGAAAAACTTCAGCAGAATGAAGCAAGAGTTCAAGAATTGAC | | |
| | CAAGGAATGGACAAATAAGTGGAATGAAACCCAAAATATTTTGAAAGAACAAACTCTA | | |
| | GCCCTCAGGAAAGAAGGGATTGGAGTTGTTTTGGATTCTGAACTGCCTCATTTGATTG | | |
| | GCATCGATGATGACCTTTTGAGTACTGGAATCATCTTATATCATTTAAAGGAAGGTCA | | |
| | GACATACGTTGGTAGAGACGATGCTTCCACGGAGCAAGATATTGTTCTTCATGGCCTT | | |
| | GACTTGGAGAGTGAGCATTGCATCTTTGAAAATATCGGGGGGACAGTGACTCTGATAC | | |
| | CCCTGAGTGGGTCCCAGTGCTCTGTGAATGGTGTTCAGATCGTGGAGGCCACACATCT | | |
| | AAATCAAGGTGCTGTGATTCTCTTGGGAAGAACCAATATGTTTCGCTTTAACCATCCA | | |
| | AAGGAAGCCGCCAAGCTCAGGGAGAAGAGGAAGAGTGGCCTTCTGTCCTCCTTCAGCT | | |
| | TGTCCATGACCGACCTCTCGAAGTCCCGTGAGAACCTGTCTGCAGTCATGTTGTATAA | | |
| | CCCCGGACTTGAATTTGAGAGGCAACAGCGTGAAGAACTTGAAAAATTAGAAAGTAAA | | |
| | AGGAAACTCATAGAAGAAATGGAGGAAAAGCAGAAATCAGACAAGGCTGAACTGGAGC | | |
| | GGATGCAGCAGGAGGTGGAGACCCAGCGCAACGAGACAGAAATCGTGCAGCTCCAGAT | | |
| | TCGCAAGCAGGAGGAGAGCCTCAAACGCCGCAGCTTCCACATCGAGAACAAGCTAAAG | | |
| | GATTTACTTGCGGAGAAGGAAAAATTTGAAGAGGAGAGGCTGAGGGAACAGCAGGAAA | | |
| | TCGAGCTGCAGAAGAAGAGACAAGAAGAAGAGACCTTTCTCCGCGTCCAAGAACAACT | | |
| | CCAACGACTCAAAGAACTCAACAACAACGAGAAGGCTGAGAAGTTTCAGATATTTCAA | | |
| | GAACTGGACCAGCTCCAAAAGGAAAAAGATGAACAGTATGCCAAGCTTGAACTGGAAA | | |
| | AAAAGAGACTAGAGGAGCAGGAGAAGGAGCAGGTCATGCTCGTGGCCCATCTGGAAGA | | |
| | GCAGCTCCGAGAGAAGCAGGAGATGATCCAGCTCCTGCCGCGTGGGGAGGTACAGTGG | | |
| | GTGGAAGAGGAGAAGAGGGACCTGGAAGGCATTCGGGAATCCCTCCTGCGGGTGAAGG | | |
| | AGGCTCGTGCCGGAGGGGATGAAGATGGCGAGGAGTTAGAAAAGGCTCAACTGCGTTT | | |
| | CTTCGAATTCAAGAGAAGGCACCTTGTCAAGCTAGTGAACTTGGAGAAGGACCTGGTT | | |
| | CAGCAGAAAGACATCCTGAAAAAAGAAGTCCAAGAAGAACAGGAGATCCTAGAGTGTT | | |
| | TAAAATGTGAACATGACAAAGAATCTAGATTGTTGGAAAAACATGATGAGAGTGTCAC | | |
| | AGATGTCACGGAAGTGCCTCAAGATTTCGAGAAAATAAAGCCAGTGGAGTACAGGCTG | | |
| | CAATATAAAGAACGCCAGCTACAGTACCTCCTGCAGAATCACTTGCCAACTCTGTTGG | | |
| | AAGAAAAGCAGAGAGCATTTGAAATTCTTGACAGAGGCCCTCTCAGCTTAGACAACAC | | |
| | TCTTTATCAAGTAGAAAAGGAAATGGAAGAAAAACAAGAACAGCTTGCACAGTACCAG | | |
| | GCCAATGCAAACCAGCTGCAAAAGCTCCAAGCCACCTTTGAATTCACTGCCAACATTG | | |
| | CACGTCAGGAGGAAAAAGTGAGGAAAAAGGAAAAGGAGATTTTGGAGTCCAGAGAGAA | | |
| | GCAGCAGAGAGAGGCGCTGGAGCGGGCCCTGGCCAGGCTGGAGAGGAGACATTCTGCG | | |
| | CTGCAGAGGCACTCCACCCTGGGCACGGAGATTGAAGAGCAGAGGCAGAAACTTGCCA | | |
| | GTCTGAACAGTGGCAGCAGAGAGCAGTCAGGGCTCCAGGCTAGCCTGGAGGCTGAGCA | | |
| | GGAAGCCCTGGAGAAGGACCAGGAGAGGTTAGAATATGAAATCCAGCAGCTGAAACAG | | |
| | AAGATTTATGAGGTCGATGGTGTTCAAAAAGATCATCATGGGACCCTGGAAGGGAAGG | | |
| | TGGCTTCTTCCAGCTTGCCAGTCAGTGCTGAAAAATCACACCTGGTTCCCCTCATGGA | | |
| | TGCCAGGATCAATGCTTACATTGAAGAAGAACTCCAAAGACGCCTTCAGGATTTGCAT | | |
| | CGTGTGATTAGTGAAGGCTGCAGTACATCTGCAGACACGATGAAGGATAATGAGAAAC | | |
| | TTCACAATGGCACCATTCAACGTAAACTAAAATATGAGCTGTGTCGTGACCTCCTGTG | | |
| | TGTCCTGATGCCAGAGCCTGATGCCGCTGCCTGCGCTAATCATCCCTTGCTCCAGCAA | | |
| | GATCTGGTTCAGCTTTCTCTTGATTGGAAAACAGAAATCCCTGATTTAGTTTTGCCAA | | |
| | ATGGAGTTCAGCTGTCATCCAAATTCCAGACTACCTTGGTTGACATGATTTACTTTCT | | |
| | TCATGGAAATATGGAAGTCAATGTCCCTTCCCTGGCAGAAGTTCAGTTACTGCTCTAC | | |
| | ACAACAGTGAAAGTCATGGGTGACTCTGGCCATGACCAGTGCCAGTCGCTAGTCCTTC | | |
| | TGAACACCCACATTGCACTGGTGAAGGAAGACTGTGTTTTTTATCCACGCATTCGATC | | |
| | TCGAAACATACCTCCTCCGGGTGCACAATTTGATGTGATCAAATGCCATGCTTTAAGT | | |
| | GAATTCAGGTGTGTTGTTGTTCCAGAAAAGAAAAATGTGTCAACAGTAGAACTAGTCT | | |
| | TCTTACAGAAACTCAAACCTTCAGTGGGTTCCAGAAATAGTCCACCTGAGCACCTTCA | | |
| | GGAAGCCCCAAATGTCCAGTTGTTCACCACCCCATTGTATCTTCAAGGCAGTCAGAAT | | |
| | GTCGCACCTGAGGTCTGGAAACTTACTTTCAATTCTCAAGATGAGGCTCTTTGGCTAA | | |
| | TCTCACATTTGACAAGACTCTAAGGAGGAGACTTTTAAAGATGCACTACATGTTTTTT | | |
| | GAGATCATTAATAAAATAAGCATTGTGAAAACAGTCAAGGCAATATGAATATCTCCGT | | |
| | GTAGCTAATTGAATTGGAACTGGAAAAATGCAGACCTCTAAAATTGAAAATGTAACTA | | |
| | TTTTAAATATCTACAATAAAATAAAAACAGCTAATAGCAGAGCCCCAATGAAATATCT | | |
| | TTATCATCACCTTGCTTCATTTTCTTGAAACTCAGGCTTGTAAATTTGTGCCTGCTTC | | |
| | ATTATTTGTGAGGTGATTAAAGCATTTCTGATTGTT | | |
| | ORF Start: ATG at 21 | | ORF Stop: TAA at 4197 |
| | SEQ ID NO:86 | 1392 aa | MW at 159799.8 kD |

| | |
|---|---|
| NOV5b, | MASVKVAVRVRPMNRREKDLEAKFITQMEKSKTTITNLKIPEGGTGDSGRERTKTFTY |
| CG106249-02 | |
| Protein Sequence | DFSFYSADTKSPDYVSQEMVFKTLGTDVVKSAFEGYNACVFAYGQTGSGKSYTMMGNS |
| | GDSGLIPRICEGLFSRINETTRWDEASFRTEVSYLEIYNERVRDLLRRKSSKTFNLRV |
| | REHPKEGPYVEDLSKHLVQNYGDVEELMDAGNINRTTAATGMNDVSSRSHAIFTTKFT |
| | QAKFDSEMPCETVSKIHLVDLAGSERADATGATGVRLKEGGNINKSLVTLGNVISALA |
| | DLSQDAANTLAKKKQVFVPYRDSVLTWLLKDSLGGNSKTIMIATISPADVNYGETLST |
| | LRYANRAKNTINKPTTNEDANVKLIRELRAEIARLKTLLAQGNQIALLDSPTALSMEE |
| | KLQQNEARVQELTKEWTNKNNETQNILKEQTLALRKEGIGVVLDSELPHLIGIDDDLL |
| | STGIILYHLKEGQTYVGRDDASTEQDIVLHGLDLESEHCIFENIGGTVTLIPLSGSQC |
| | SVNGVQIVEATHLNQGAVILLGRTNMFRFNHPKEAAKLREKRKSGLLSSFSLSMTDLS |
| | KSRENLSAVMLYNPGLEFERQQREELEKLESKRKLIEEMEEKQKSDKAELERMQQEVE |
| | TQRKETEIVQLQTRKQEESLKRRSFHIENKLKDLLAEKEKFEEERLREQQEIELQKKR |
| | QEEETFLRVQEELQRLKELNNNEKAEKFQIFQELDQLQKEKDEQYAKLELEKKRLEEQ |
| | EKEQVMLVAHLEEQLREKQEMIQLLRRGEVQWVEEEKRDLEGIRESLLRVKEARAGGD |
| | EDGEELEKAQLRFFEFKRRQLVKLVNLEKDLVQQKDILKKEVQEEQEILECLKCEHDK |
| | ESRLLEKHDESVTDVTEVPQDFEKIKPVEYRLQYKERQLQYLLQNHLPTLLEEKQRAF |
| | EILDRGPLSLDNTLYQVEKEMEEKEEQLAQYQANANQLQKLQATFEFTANIARQEEKV |
| | RKKEKEILESREKQQREALERALARLERRHSALQRHSTLGTEIEEQRQKLASLNSGSR |
| | EQSGLQASLEAEQEALEKDQERLEYEIQQLKQKIYEVDGVQKDHHGTLEGKVASSSLP |
| | VSAEKSHLVPLMDARINAYIEEEVQRRLQDLHRVISEGCSTSADTMKDNEKLHNGTIQ |
| | RKLKYELCRDLLCVLMPEPDAAACANHPLLQQDLVQLSLDWKTEIPDLVLPMGVQVSS |
| | KPQTTLVDMIYFLHGNNEVNVPSLAEVQLLLYTTVKVMGDSGHDQCQSLVLLNTHIAL |
| | VKEDCVFYPRIRSRNIPPPGAQFDVIKCHALSEFRCVVVPEKKNVSTVELVFLQKLKP |
| SVGSRNSPPEHLQEAPNVQLFTTPLYLQGSQNVAPEVWKLTFNSQDEALWLISHLTRL | |

Sequence comparison of the above protein sequences yields the following sequence relationships shown in Table 5B.

TABLE 5B
Comparison of NOV5a against NOV5b.

| | | Identities/ |
|---|---|---|
| | | Similarities for |
| Protein | NOV5a Residues/ | the Matched |
| Sequence | Match Residues | Region |
| NOV5b | 1 . . . 1394 | 1375/1394 (98%) |
| | 1 . . . 1392 | 1379/1394 (98%) |

Further analysis of the NOV5a protein yielded the following properties shown in Table 5C.

TABLE 5C
Protein Sequence Properties NOV5a

| | |
|---|---|
| PSort | 0.6086 probability located in mitochondrial matrix space; |
| analysis: | 0.3127 probability located in mitochondrial inner membrane; |
| | 0.3127 probability located in mitochondrial intermembrane |
| | space; 0.3127 probability located in mitochondrial outer |
| | membrane |
| SignalP | No Known Signal Sequence Predicted |
| analysis: | |

A search of the NOV5a protein against the Geneseq database, a proprietary database that contains sequences published in patents and patent publication, yielded several homologous proteins shown in Table 5D.

TABLE 5D
Geneseq Results for NOV5a

| | | NOV5a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB79531 | Human kinesin motor protein | 1 . . . 1394 | 1358/1394 (97%) | 0.0 |
| | HsKif16b - Homo sapiens, 1375 | 1 . . . 1375 | 1362/1394 (97%) | |
| | aa. [US6399346-B1, 04 JUN. | | | |
| | 2002] | | | |
| AAE22525 | Human HsKif16b protein - Homo | 1 . . . 1394 | 1358/1394 (97%) | 0.0 |
| | sapiens, 1375 aa. [US6355471-B1, | 1 . . . 1375 | 1362/1394 (97%) | |
| | 12 MAR. 2002] | | | |
| ABB79530 | Human kinesin motor protein | 1 . . . 359 | 347/359 (96%) | 0.0 |
| | HsKif16b motor domain - Homo | 1 . . . 359 | 350/359 (96%) | |
| | sapiens, 359 aa. [US6399346-B1, | | | |
| | 04 JUN. 2002] | | | |
| AAE22526 | Human HsKif16b motor domain | 1 . . . 359 | 347/359 (96%) | 0.0 |
| | fragment - Homo sapiens, 359 aa. | 1 . . . 359 | 350/359 (96%) | |
| | [US6355471-B1, 12 MAR. 2002] | | | |
| ABB61704 | Drosophila melanogaster | 20 . . . 757 | 350/776 (45%) | e−161 |
| | polypeptide SEQ ID NO 11904 - | 1 . . . 737 | 476/776 (61%) | |
| | Drosophila melanogaster, 1174 aa. | | | |
| | [WO200171042-A2, 27 SEP. 2001] | | | |

In a BLAST search of public sequence datbases, the NOV5a protein was found to have homology to the proteins shown in the BLASTP data in Table 5E.

TABLE 5E
Public BLASTP Results for NOV5a

| | | NOV5a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9HCI2 | KIAA1590 protein - Homo sapiens | 155 . . . 1394 | 1233/1240 (99%) | 0.0 |
| | (Human), 1238 aa (fragment). | 1 . . . 1238 | 1234/1240 (99%) | |
| Q9BQM0 | DJ971B4.1.2 (KIAA1590 (Novel | 596 . . . 1394 | 791/799 (98%) | 0.0 |
| | protein similar to KIF1 type and | 1 . . . 797 | 792/799 (98%) | |
| | other kinesin-like proteins) | | | |
| | (Isoform 2)) - Homo sapiens | | | |
| | (Human), 797 aa (fragment). | | | |
| Q9NXN9 | CDNA FLJ20135 fis, clone | 202 . . . 953 | 747/752 (99%) | 0.0 |
| | COL06818 - Homo sapiens | 1 . . . 752 | 750/752 (99%) | |
| | (Human), 752 aa (fragment). | | | |
| Q9BQM1 | DJ971B4.1.1 (KIAA1590 (Novel | 596 . . . 1168 | 565/573 (98%) | 0.0 |
| | protein similar to KIF1 type and | 1 . . . 571 | 566/573 (98%) | |
| | other kinesin-like proteins) | | | |
| | (Isoform 1)) - Homo sapiens | | | |
| | (Human), 722 aa (fragment). | | | |
| Q9BQM5 | DJ777L9.1 (KIAA1590 (Novel | 37 . . . 434 | 378/398 (94%) | 0.0 |
| | protein similar to KIF1 type and | 37 . . . 429 | 382/398 (95%) | |
| | other kinesin-like proteins)) - Homo | | | |
| | sapiens (Human), 429 aa | | | |
| | (fragment). | | | |

PFam analysis predicts that the NOV5a protein contains the domains shown in the Table 5F.

TABLE 5F
Domain Analysis of NOV5a

| Pfam | NOV5a | Identities/Similarities | Expect |
|---|---|---|---|
| Domain | Match Region | for the Matched Region | Value |
| kinesin | 9 . . . 387 | 187/421 (44%) | 3.8e−152 |
| | | 301/421 (71%) | |
| FHA | 478 . . . 544 | 21/80 (26%) | 0.025 |
| | | 45/80 (56%) | |

### Example 6

The NOV6 clone was analyzed, and the nucleotide and encoded polypeptide sequences are shown in Table 6A.

TABLE 6A
NOV6 Sequence Analysis

| | SEQ ID NO:87 | 858 bp | |
|---|---|---|---|
| NOV6a, | GCCCACGATGCTCCTCCTTGCTCCCCAGATGCTGAATCTGCTGCTGCTGGCGCTGCCC | | |
| CG106824-01 | | | |
| DNA Sequence | GTCCTGGCGAGCCGCCCCTACGCGGCCCCTCCAGCCCCAGGCCAGGCCCTGCAGCGAG | | |
| | TGGGCATCGTCGGGGGTCAGGAGGCCCCCAGGAGCAAGTGGCCCTGGCAGGTGAGCCT | | |
| | GAGAGTCCACGGCCCATACTGGATGCACTTCTGCGGGGGCTCCCTCATCCACCCCCAG | | |
| | TGGGTGCTGACCGCAGCGCACTGCGTGGGACCGGACGTCAAGGATCTGGCCGCCCTCA | | |
| | GGGTGCAACTGCGGGAGCAGCACCTCTACTACCAGGACCAGCTGCTGCCGGTCAGCAG | | |
| | GATCATCGTGCACCCACAGTTCTACACCGCCCAGATCGGAGCGGACATCGCCCTGCTG | | |
| | GAGCTGGAGGAGCCGGTGAACGTCTCCAGCCACGTCCACACGGTCACCCTGCCCCCTG | | |
| | CCTCAGAGACCTTCCCCCCGGGGATGCCGTGCTGGGTCACTGGCTGGGGCGATGTGCT | | |
| | CCCACCGCCATTTCCTCTGAAGCAGGTGAAGGTCCCCATAATGGAAAACCACATTTGT | | |
| | GACGCAAAATACCACCTTGGCGCCTACACGGGAGACGACGTCCGCATCGTCCGTGACG | | |
| | ACATGCTGTGTGCCGGGAACACCCGGAGGGACTCATGCCAGCAGGGCGACTCCGGAGG | | |
| | GCCCCTGGTGTGCAAGGTGAATGGCACCTGGCTGCAGGCGGGCGTGGTCAGCTGGGGC | | |
| | GAGGGCTGTGCCCAGCCCAACCGGCCTGGCATCTACACCCGTGTCACCTACTACTTGG | | |
| | ACTGGATCCACCACTATGTCCCCAAAAAGCCGTGAGTCAGGCCTGG | | |
| | ORF Start: ATG at 8 | | ORF Stop: TGA at 845 |
| | SEQ ID NO:88 | 279 aa | MW at 30877.5 kD |
| NOV6a, | MLLLAPQMLNLLLLALPVLASRAYAAPPAPGQALQRVGIVGGQEAPRSKWPWQVSLRV | | |
| CG106824-01 | | | |
| Protein Sequence | HGPYWMHFCGGSLIHPQNVLTAAHCVGPDVKDLAALRVQLREQHLYYQDQLLPVSRII | | |
| | VHPQFYTAQIGADIALLELEEPVNVSSHVHTVTLPPASETFPPGMPCWVTGWGDVLPP | | |
| | PFPLKQVKVPIMENHICDAKYHLGAYTGDDVRTVRDDMLCAGNTRRDSCQQGDSGGPL | | |
| | VCKVNGTWLQAGVVSWGEGCAQPNRPGIYTRVTYYLDWIHHYVPKKP | | |
| | SEQ ID NO:89 | 1828 bp | |
| NOV6b, | ATGCTGAGCCTGCTGCTGCTGGCGCTGCCCGTCCTGGCGAGCCCGGCCTACGTGGCCC | | |
| CG106824-04 | | | |
| DNA Sequence | CTGCCCCAGGCCAGGCCCTGCAGCAAACGGGCATTGTTGGGGGGCAGGAGGCCCCCAG | | |
| | GAGCAAGTGGCCCTGGCAGGTGAGCCTGAGAGTCCGCGGCCCATACTGGATGCACTTC | | |
| | TGCGGGGGCTCCCTCATCCACCCCCAGTGGGTGCTAACCGCGGCGCACTGCGTGGAAC | | |
| | CGGACATCAAGGATCTGGCCCCCCTCAGGGTGCAACTGCGGGAGCAGCACCTCTACTA | | |
| | CCAGGACCAGCTGCTGCCGGTCAGCAGGATCATCGTGCACCCACAGTTCTACATCATC | | |
| | CAGACCGGGGCGGACATCGCCCTGCTGGAGCTGGAGGAGCCCGTGAACATCTCCAGCC | | |
| | ACATCCACACGGTCACGCTGCCCCCTGCCTCGGAGACCTTCCCCCCGGGGATGCCGTG | | |
| | CTGGGTCACTCGCTGGGGCGACGTGGACAATAATGAGCGCCTCCCACCGCCATTTCCT | | |
| | CTGAAGCAGGTGAAGGTCCCCATAATGGAAAACCACATTTGTGACGCAAAATACCACC | | |
| | TTGGCCCCTACACGGGAGACGACGTCCGCATCGTCCGTGACGACATGCTGTGTGCCGG | | |
| | GAACACCCGGAGGGACTCATGCCAGGGCGACTCCGGAGGGCCCCTGGTGTGCAAGGTG | | |
| | AATGGCACCTGGCTGCAGGCGGGCGTGGTCAGCTGGGGCGAGGGCTGTGCCCAGCCCA | | |
| | ACCGGCCTGGCATCTACACCCGTGTCACCTACTACTTGGACTGGATCCACCACTATGT | | |
| | CCCCAAAAAGCCGTGA | | |
| | ORF Start: ATG at 1 | | ORF Stop: TGA at 826 |
| | SEQ ID NO:90 | 275 aa | MW at 30605.0 kD |
| NOV6b, | MLSLLLLALPVLASPAYVAPAPGQALQQTGIVGGQEAPRSKWPWQVSLRVRGPYWMHF | | |
| CG106824-04 | | | |
| Protein Sequence | CGGSLIHPQWVLTAAHCVEPDIKDLAALRVQLREQHLYYQDQLLPVSRIIVHPQFYII | | |
| | QTGADIALLELEEPVNISSHIHTVTLPPASETFPPGMPCWVTGWGDVDNNERLPPPFP | | |
| | LKQVKVPIMENHICDAKYHLGAYTGDDVRIVRDDMLCAGNTRRDSCQGDSGGPLVCKV | | |
| | NGTWLQAGVVSWGEGCAQPNRPGIYTRVTYYLDWIHHYVPKKP | | |
| | SEQ ID NO:91 | 828 bp | |
| NOV6c, | ATGCTGAATCTGCTGCTGCTGGCGCTGCCCGTCCTGGCGAGCCGCGCCTACGCGGCCC | | |
| CG106824-02 | | | |
| DNA Sequence | CTGCCCCAGGCCAGGCCCTGCAGCGAGTGGGCATCGTCGGGGGTCAGGAGGCCCCCAG | | |
| | GAGCAAGTGGCCCTGGCAGGTGAGCCTGAGAGTCCACGGCCCATACTGGATGCACTTC | | |
| | TGCGGGGGCTCCCTCATCCACCCCCAGTGGGTGCTGACCGCAGCGCACTGCGTGGGAC | | |
| | CGGACGTCAAGGATCTGGCCGCCCTCAGGGTGCAACTGCGGGAGCAGCACCTCTACTA | | |
| | CCAGGACCACCTGCTGCCGGTCAGCAGGATCATCGTGCACCCACAGTTCTACACCGCC | | |
| | CAGATCGGAGCGGACATCGCCCTGCTGGAGCTGGAGGAGCCGGTGAACGTCTCCAGCC | | |
| | ACGTCCACACGGTCACCCTCCCCCCTGCCTCAGAGACCTTCCCCCCGGGGATGCCGTG | | |
| | CTGGGTCACTGGCTGGGGCGATGTGGACAATGATGAGCGCCTCCCACCGCCATTTCCT | | |
| | CTGAAGCAGGTGAAGGTCCCCATAATGCAAAACCACATTTGTGACGCAAAATACCACC | | |
| | TTGGCGCCTACACGGGAGACGACGTCCCCATCGTCCGTGACGACATGCTGTGTGCCGG | | |
| | GAACACCCGGAGGGACTCATGCCAGGGCGACTCCGGAGGGCCCCTGGTGTGCAAGGTG | | |
| | AATGGCACCTGGCTGCAGGCGGGCGTGGTCAGCTGGGGCGAGGGCTGTGCCCAGCCCA | | |
| | ACCGGCCTGGCATCTACACCCGTGTCACCTACTACTTGGACTGGATCCACCACTATGT | | |
| | CCCCAAAAAGCCGTGA | | |
| | ORF Start: ATG at 1 | | ORF Stop: TGA at 826 |
| | SEQ ID NO:92 | 275 aa | MW at 30514.9 kD |
| NOV6c, | MLNLLLLALPVLASRAYAAPAPGQALQRVGIVGGQEAPRSKWPWQVSLRVHGPYWMHF | | |
| CG106824-02 | | | |
| Protein Sequence | CGGSLIHPQWVLTAAHCVGPDVKDLAALRVQLREQHLYYQDQLLPVSRIIVHPQFYTA | | |
| | QIGADIALLELEEPVNVSSHVHTVTLPPASETFPPGMPCWVTGWGDVDNDERLPPPFP | | |
| | LKQVKVPIMENHICDAKYHLGAYTGDDVRIVRDDMLCAGNTRRDSCQGDSGGPLVCKV | | |
| | NGTWLQAGVVSWGEGCAQPNRPGIYTRVTYYLDWIHHYVPKKP | | |
| | SEQ ID NO:93 | 1145 bp | |
| NOV6d, | GGCCAGGATGCTGAATCTGCTGCTGCTGGCGCTGCCCGTCCTGGCGAGCCGCGCCTAC | | |
| CG106824-03 | | | |
| DNA Sequence | GCGGCCCCTGCCCCAGGCCAGGCCCTGCAGCGAGTGGGCATCGTTGGGGGTCAGGAGG | | |
| | CCCCCAGGAGCAAGTGGCCCTGGCAGGTGAGCCTGAGAGTCCACGGCCCATACTGGAT | | |
| | GCACTTCTGCGGGGGCTCCCTCATCCACCCCCAGTGGGTGCTGACCGCAGCGCACTGC | | |
| | GTGGGACCGGACGTCAAGGATCTGGCCGCCCTCAGGGTGCAACTGCGGGAGCAGCACC | | |
| | TCTACTACCAGGACCAGCTGCTGCCGGTCAGCAGGATCATCGTGCACCCACAGTTCTA | | |
| | CACCGCCCAGATCGGAGCGGACATCGCCCTGCTGGAGCTGGAGGAGCCGGTGAAGGTC | | |
| | TCCAGCCACGTCCACACGGTCACCCTGCCCCCTGCCTCAGAGACCTTCCCCCCGGGGA | | |
| | TGCCGTGCTGGGTCACTGGCTGGGGCGATGTGGACAATGATGAGCGCCTCCCACCGCC | | |
| | ATTTCCTCTGAAGCAGGTGAAGGTCCCCATAATGGAAAACCACATTTGTGACGCAAAA | | |
| | TACCACCTTGGCGCCTACACGGGAGACGACGTCCGCATCGTCCGTGACGACATGCTGT | | |
| | GTGCCGGGAACACCCGGAGGGACTCATGCCAGGGCGACTCCGGAGGGCCCCTGGTGTG | | |
| | CAAGGTCAATGGCACCTCGCTGCAGGCGGGCGTGGTCAGCTGGGGCGAGGGCTGTGCC | | |
| | CAGCCCAACCGGCCTGGCATCTACACCCGTGTCACCTACTACTTGGACTGGATCCACC | | |
| | ACTATGTCCCCAAAAAGCCGTGAGTCAGGCCTGGGTTGGCCACCTGGGTCACTGGAGG | | |
| | ACCAACCCCTGCTGTCCAAAACACCACTGCTTCCTACCCAGGTGGCGACTGCCCCCCA | | |
| | CACCTTCCCTGCCCCGTCCTGAGTGCCCCTTCCTGTCCTAAGCCCCCTGCTCTCTTCT | | |
| | GAGCCCCTTCCCCTGTCCTGAGGACCCTTCCCCATCCTGAGCCCCCTTCCCTGTCCTA | | |
| | AGCCTGACGCCTGCACCGGGCCCTCCGGCCCTCCCCTGCCCAGGCAGCTCGTGGTGGG | | |
| | CGCTAATCCTCCTGAGTGCTGGACCTCATTAAAGTGCATGGAA | | |
| | ORF Start: ATG at 8 | | ORF Stop: TGA at 833 |
| | SEQ ID NO:94 | 275 aa | MW at 30528.9 kD |

| | |
|---|---|
| NOV6d, | MLNLLLLALPVLASRAYAAPAPGQALQRVGIVGGQEAPRSKWPWQVSLRVHGPYWMHF |
| CG106824-03 | |
| Protein Sequence | CGGSLIHPQWVLTAAHCVGPDVKDLAALRVQLREQHLYYQDQLLPVSRIIVHPQFYTA |
| | QIGADIALLELEEPVKVSSHVHTVTLPPASETFPPGMPCWVTGWGDVDNDERLPPPFP |
| | LKQVKVPIMENHICDAKYHLGAYTGDDVRIVRDDMLCAGNTRRDSCQGDSGGPLVCKV |
| | NGTWLQAGVVSWGEGCAQPNRPGIYTRVTYYLDWIHHYVPKKP |

Sequence comparison of the above protein sequences yields the following sequence relationships shown in Table 6B.

TABLE 6B
Comparison of NOV6a against NOV6b through NOV6d.

| Protein | NOV6a Residues/ | Identities/Similarities |
|---|---|---|
| Sequence | Match Residues | for the Matched Region |
| NOV6b | 8 . . . 279 | 257/277 (92%) |
| | 1 . . . 275 | 262/277 (93%) |
| NOV6c | 8 . . . 279 | 270/277 (97%) |
| | 1 . . . 275 | 270/277 (97%) |
| NOV6d | 8 . . . 279 | 269/277 (97%) |
| | 1 . . . 275 | 269/277 (97%) |

Further analysis of the NOV6a protein yielded the following properties shown in Table 6C.

TABLE 6C
Protein Sequence Properties NOV6a

| | |
|---|---|
| PSort | 0.8650 probability located in lysosome (lumen); 0.6950 |
| analysis: | probability located in outside; 0.1333 probability located |
| | in microbody (peroxisome); 0.1000 probability located in |
| | endoplasmic reticulum (membrane) |
| SignalP | Cleavage site between residues 21 and 22 |
| analysis: | |

A search of the NOV6a protein against the Geneseq database, a proprietary database that contains sequences published in patents and patent publication, yielded several homologous proteins shown in Table 6D.

TABLE 6D
Geneseq Results for NOV6a

| | | NOV6a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAW63174 | Human mast cell tryptase I | 10 . . . 279 | 268/275 (97%) | e−161 |
| | polypeptide - Homo sapiens, 273 | 1 . . . 273 | 268/275 (97%) | |
| | aa. [WO9833812-A1, 06 AUG. | | | |
| | 1998] | | | |
| AAW64238 | Human mast cell tryptase I - Homo | 10 . . . 279 | 268/275 (97%) | e−161 |
| | sapiens, 273 aa. [WO9824886-A1, | 1 . . . 273 | 268/275 (97%) | |
| | 11 JUN. 1998] | | | |
| AAW63175 | Human mast cell tryptase II/beta | 9 . . . 279 | 268/276 (97%) | e−161 |
| | polypeptide - Homo sapiens, 274 | 1 . . . 274 | 268/276 (97%) | |
| | aa. [WO9833812-A1, 06 AUG. | | | |
| | 1998] | | | |
| AAW64240 | Human mast cell tryptase II/beta - | 9 . . . 279 | 268/276 (97%) | e−161 |
| | Homo sapiens, 274 aa. | 1 . . . 274 | 268/276 (97%) | |
| | [WO9824886-A1, 11 JUN. | | | |
| | 1998] | | | |
| AAE14348 | Human protease PRTS-13 protein - | 1 . . . 279 | 263/278 (94%) | e−157 |
| | Homo sapiens, 691 aa. | 10 . . . 283 | 264/278 (94%) | |
| | [WO200183775-A2, 08 NOV. | | | |
| | 2001] | | | |

In a BLAST search of public sequence datbases, the NOV6a protein was found to have homology to the proteins shown in the BLASTP data in Table 6E.

TABLE 6E
Public BLASTP Results for NOV6a

| | | NOV6a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q15661 | Tryptase beta-1 precursor (EC | 8 . . . 279 | 270/277 (97%) | e−162 |
| | 3.4.21.59) (Tryptase 1) (Tryptase | 1 . . . 275 | 270/277 (97%) | |
| | I) - Homo sapiens (Human), 275 aa. | | | |
| P20231 | Tryptase beta-2 precursor (EC | 8 . . . 279 | 269/277 (97%) | e−161 |
| | 3.4.21.59) (Tryptase 2) (Tryptase | 1 . . . 275 | 269/277 (97%) | |
| | II) - Homo sapiens (Human), 275 | | | |
| | aa. | | | |
| C35863 | tryptase (EC 3.4.21.59) III | 8 . . . 279 | 267/277 (96%) | e−159 |
| | precursor - human, 275 aa. | 1 . . . 275 | 267/277 (96%) | |
| Q96RZ6 | Tryptase I - Homo sapiens | 8 . . . 279 | 266/277 (96%) | e−159 |
| | (Human), 275 aa. | 1 . . . 275 | 267/277 (96%) | |
| P15157 | Alpha-tryptase precursor (EC | 8 . . . 279 | 252/277 (90%) | e−150 |
| | 3.4.21.59) (Tryptase 1) - Homo | 1 . . . 275 | 258/277 (92%) | |
| | sapiens (Human), 275 aa. | | | |

PFam analysis predicts that the NOV6a protein contains the domains shown in the Table 6F.

TABLE 6F
Domain Analysis of NOV6a

| | NOV6a | Identities/Similarities | Expect |
|---|---|---|---|
| Pfam Domain | Match Region | for the Matched Region | Value |
| trypsin | 39 . . . 271 | 111/264 (42%) | 6.4e−89 |
| | | 191/264 (72%) | |

### Example 7

The NOV7 clone was analyzed, and the nucleotide and encoded polypeptide sequences are shown in Table 7A.

TABLE 7A
NOV7 Sequence Analysis

| | SEQ ID NO:95 | 842 bp | |
|---|---|---|---|
| NOV7a, | GTGGCCGTCCGAGAGCCGAGAGGTGAGGGTGCCCCCGCCTCACCTGCAGAGGGGCCGT | | |
| CG114327-01 | | | |
| DNA Sequence | TCCGGGCTCGAACCCGGCACCTTCCGGAAAATGGCGGCTGCCAGGCCCAGCCTGGGCC | | |
| | GAGTCCTCCCAGGATCCTCTGTCCTGTTCCTGTGTGACATGCAGGAGAAGTTCCGCCA | | |
| | CAACATCGCCTACTTCCCACAGATCGTCTCAGTGGCTGCCCGCATGCTCAAGAACACG | | |
| | ACCCTGGACCTCCTACACCGGGGGCTGCAGGTCCATGTGGTGGTGGACGCCTGCTCCT | | |
| | CACGCAGCCAGGTGGACCGGCTGGTGGCTCTGGCCCGCATGAGACAGAGTGGTGCCTT | | |
| | CCTCTCCACCAGCGAAGGGCTCATTCTGCAGCTTGTGGGCGATGCCGTCCACCCCCAG | | |
| | TTCAAGGAGATCCAGAAACTCATCAAGGAGCCCGCCCCAGACAGCGGACTGCTGGGCC | | |
| | TCTTCCAAGGCCAGAACTCCCTCCTCCACTGAACTCCAACCCTGCCTTGAGGGAAGAC | | |
| | CACCCTCCTGTCACCCGGACCTCAGTGGAAGCCCGTTCCCCCCATCCCTGGATCCCAA | | |
| | GAGTGGTGCGATCCACCAGGAGTGCCGCCCCCTTGTGGGGGGGGGCAGGGTCCTGCCT | | |
| | TCCCATTGGACAGCTGCTCCCGGAAATGCAAATGAGACTCCTGGAAACTGGGTGGGAA | | |
| | TTGGCTGAGCCAAGATGGAGGCGGGGCTCGGCCCCGGGCCACTTCACGGGGCGGGAAG | | |
| | GGGAGGGGAAGAAGAGTCTCAGACTGTGGGACACGGACTCGCAGAATAAACATATATG | | |
| | TGGCAAAAAAAAAAAAAAAAAAAAAAAAAA | | |
| | ORF Start: ATG at 89 | | ORF Stop: TGA at 494 |
| | SEQ ID NO:96 | 135 aa | MW at 14765.0 kD |
| NOV7a, | MAAARPSLGRVLPGSSVLFLCDMQEKFRHNIAYFPQIVSVAARMLKNTTLDLLDRGLQ | | |
| CG114327-01 | | | |
| Protein Sequence | VHVVVDACSSRSQVDRLVALARMRQSGAFLSTSEGLILQLVGDAVHPQFKEIQKLIKE | | |
| | PAPDSGLLGLFQGQNSLLH | | |
| | SEQ ID NO:97 | 1091 bp | |
| NOV7b, | GAAACGGTAACCAGCCCTGGGAAGCCCGCAAGAGGCCTCAGCGGTGGCCGTCCGAGCG | | |
| CG114327-02 | | | |
| DNA Sequence | CCGAGAGGTGAGGGTGCCCCCGCCTCACCTGCAGAGGGGCCGTTCCGGGCTCGAACCC | | |
| | GGCACCTTCCGGAAAATGGCGGCTGCCAGGCCCAGCCTGGGCCGAGTCCTCCCAGGAT | | |
| | CCTCTGTCCTGTTCCTCTGTGACATGCAGCAGAAGTTCCGCCACAACATCGCCTACTT | | |
| | CCCACAGATCGTCTCAGTGGCTGCCCGCATGCTCAAGGTGGCCCGGCTGCTTGAGGTG | | |
| | CCAGTCATGCTGACGGAGCAGTACCCACAAGGCCTGGGCCCCACGGTGCCCGAGCTGG | | |
| | GGACTGAGGGCCTTCGGCCGCTGGCCAAGACCTGCTTCAGCATGGTGCCTGCCCTGCA | | |
| | GCAGGAGCTGGACAGTCGGCCCCAGCTGCGCTCTGTGCTGCTCTGTGGCATTGAGGCA | | |
| | CAGGCCTGCATCTTGAACACGACCCTGGACCTCCTAGACCGGGGGCTGCACGTCCATG | | |
| | TGGTGGTGGACGCCTGCTCCTCACGCAGCCAGGTGGACCGGCTGGTGGCTCTGGCCCG | | |
| | CATGAGACAGAGTGGTGCCTTCCTCTCCACCAGCGAAGGGCTCATTCTGCAGCTTGTG | | |
| | GGCGATGCCGTCCACCCCCAGTTCAAGGACATCCAGAAACTCATCAAGGAGCCCGCCC | | |
| | CAGACAGCGGACTGCTGGCCCTCTTCCAAGGCCAGAACTCCCTCCTCCACTGAACTCC | | |
| | AACCCTGCCTTGAGGGAAGACCACCCTCCTGTCACCCGGACCTCAGTGGAAGCCCGTT | | |
| | CCCCCCATCCCTGGATCCCAAGAGTGGTGCGATCCACCAGGAGTGCCGCCCCCTTGTG | | |
| | GGGGGGGGCAGGGTGCTGCCTTCCCATTGGACAGCTCCTCCCGGAAATGCAAATGAGA | | |
| | CTCCTGGAAACTGGGTGGGAATTGGCTGACCCAAGATGGACGCGGGGCTCGGCCCCGG | | |
| | GCCACTTCACGGGGCGGGAAGGGGAGGGGAAGAAGAGTCTCAGACTGTGGGACACGGA | | |
| | CTCGCAGAATAACATATATGTGGCTGTGAAAAAAAAAAAAAAAAAA | | |
| | ORF Start: ATG at 132 | | ORF Stop: TGA at 747 |
| | SEQ ID NO:98 | 205 aa | MW at 22336.9 kD |

| | |
|---|---|
| NOV7b, | MAAARPSLGRVLPGSSVLFLCDMQEKFRHNIAYFPQIVSVAARMLKVARLLEVPVMLT |
| CG114327-02 | |
| Protein Sequence | EQYPQGLGPTVPELGTEGLRPLAKTCFSMVPALQQELDSRPQLRSVLLCGIEAQACIL |
| | NTTLDLLDRGLQVHVVVDACSSRSQVDRLVALARMRQSGAFLSTSEGLILQLVGDAVH |
| | PQFKEIQKLIKEPAPDSGLLGLFQGQNSLLH |

Sequence comparison of the above protein sequences yields the following sequence relationships shown in Table 7B.

TABLE 7B
Comparison of NOV7a against NOV7b.

| Protein | NOV7a Residues/ | Identities/Similarities |
|---|---|---|
| Sequence | Match Residues | for the Matched Region |
| NOV7b | 35 . . . 135 | 94/107 (87%) |
| | 99 . . . 205 | 96/107 (88%) |

Further analysis of the NOV7a protein yielded the following properties shown in Table 7C.

TABLE 7C
Protein Sequence Properties NOV7a

| | |
|---|---|
| PSort | 0.5108 probability located in mitochondrial |
| analysis: | matrix space; 0.4500 probability located in |
| | cytoplasm; 0.2553 probability located in |
| | lysosome (lumen); 0.2357 probability |
| | located in mitochondrial inner membrane |
| SignalP | Cleavage site between residues 24 and 25 |
| analysis: | |

A search of the NOV7a protein against the Geneseq database, a proprietary database that contains sequences published in patents and patent publication, yielded several homologous proteins shown in Table 7D.

TABLE 7D
Geneseq Results for NOV7a

| | | | Identities/ | |
|---|---|---|---|---|
| | | | Similarities for | |
| Geneseq | Protein/Organism/Length | NOV7a Residues/ | the Matched | Expect |
| Identifier | [Patent #, Date] | Match Residues | Region | Value |
| AAM41577 | Human polypeptide SEQ ID NO | 1 . . . 135 | 135/135 (100%) | 5e−71 |
| | 6508 - Homo sapiens, 173 aa. | 39 . . . 173 | 135/135 (100%) | |
| | [WO200153312-A1, 26 JUL. 2001] | | | |
| AAM39791 | Human polypeptide SEQ ID NO | 1 . . . 135 | 135/135 (100%) | 5e−71 |
| | 2936 - Homo sapiens, 135 aa. | 1 . . . 135 | 135/135 (100%) | |
| | [WO200153312-A1, 26 JUL. 2001] | | | |
| AAU23364 | Novel human enzyme polypeptide | 6 . . . 133 | 122/128 (95%) | 5e−63 |
| | #450 - Homo sapiens, 162 aa. | 27 . . . 154 | 123/128 (95%) | |
| | [WO200155301-A2, 02 AUG. | | | |
| | 2001] | | | |
| AAB42186 | Human ORFX ORF 1950 | 6 . . . 135 | 99/136 (72%) | 1e−44 |
| | polypeptide sequence SEQ ID | 114 . . . 249 | 105/136 (76%) | |
| | NO: 3900 - Homo sapiens, 249 aa. | | | |
| | [WO200058473-A2, 05 OCT. | | | |
| | 2000] | | | |
| AAG89278 | Human secreted protein, SEQ ID | 35 . . . 135 | 94/107 (87%) | 3e−44 |
| | NO: 398 - Homo sapiens, 205 aa. | 99 . . . 205 | 96/107 (88%) | |
| | [WO200142451-A2, 14 JUN. 2001] | | | |

In a BLAST search of public sequence datbases, the NOV7a protein was found to have homology to the proteins shown in the BLASTP data in Table 7E.

TABLE 7E
Public BLASTP Results for NOV7a

| | | | Identities/ | |
|---|---|---|---|---|
| Protein | | | Similarities for | |
| Accession | | NOV7a Residues/ | the Matched | Expect |
| Number | Protein/Organism/Length | Match Residues | Portion | Value |
| Q96AB3 | Similar to hypothetical protein | 35 . . . 135 | 94/107 (87%) | 8e−44 |
| | FLJ23469 - Homo sapiens | 99 . . . 205 | 96/107 (88%) | |
| | (Human), 205 aa. | | | |
| Q9H5G0 | CDNA: FLJ23469 fis, clone | 46 . . . 135 | 89/90 (98%) | 1e−43 |
| | HSI11914 - Homo sapiens | 132 . . . 221 | 90/90 (99%) | |
| | (Human), 221 aa. | | | |
| Q9D8T8 | 0610042E07Rik protein - Mus | 47 . . . 134 | 69/89 (77%) | 8e−31 |
| | musculus (Mouse), 131 aa. | 38 . . . 126 | 78/89 (87%) | |
| Q9DCC7 | 0610042E07Rik protein - Mus | 47 . . . 134 | 69/89 (77%) | 8e−31 |
| | musculus (Mouse), 210 aa. | 117 . . . 205 | 78/89 (87%) | |
| Q20062 | F35G2.2 protein - Caenorhabditis | 48 . . . 126 | 50/79 (63%) | 1e−19 |
| | elegans, 199 aa. | 118 . . . 196 | 59/79 (74%) | |

PFam analysis predicts that the NOV7a protein contains the domains shown in the Table 7F.

TABLE 7F
Domain Analysis of NOV7a

| | | Identities/ | |
|---|---|---|---|
| | | Similarities for | |
| Pfam | NOV7a | the Matched | Expect |
| Domain | Match Region | Region | Value |
| Isochorismatase | 13 . . . 126 | 22/213 (10%) | 0.61 |
| | | 86/213 (40%) | |

### Example 8

The NOV8 clone was analyzed, and the nucleotide and encoded polypeptide sequences are shown in Table 8A.

TABLE 8A
NOV8 Sequence Analysis

| | SEQ ID NO:99 | 1349 bp | |
|---|---|---|---|
| NOV8a, | TGCGCCAGGATGGAGTTCGTGAAATGCCTTGGCCACCCCGAAGAGTTCTACAACCTGG | | |
| CG119418-01 | | | |
| DNA Sequence | TGCGCTTCCGGATCGGGGGCAAGCGGAAGGTGATGCCCAAGATGGACCAGGACTCGCT | | |
| | CAGCAGCAGCCTGAAAACTTGCTACAAGTATCTCAATCAGACCAGTCGCAGTTTCGCA | | |
| | GCTGTTATCCAGGCGCTGGATGGGGAAATGCGCAACGCAGTGTGCATATTTTATCTGG | | |
| | TTCTCCGAGCTCTGGACACACTGGAAGATGACATGACCATCAGTGTGGAAAAGAAGGT | | |
| | CCCGCTGTTACACAACTTTCACTCTTTCCTTTACCAACCAGACTGGCGGTTCATGGAG | | |
| | AGCAAGGAGAAGGATCGCCAGGTGCTGGAGGACTTCCCAACGATCTCCCTTGAGTTTA | | |
| | GAAATCTGGCTGAGAAATACCAAACAGTGATTGCCGACATTTGCCGGAGAATGGGCAT | | |
| | TGGGATGGCAGAGTTTTTGGATAAGCATGTGACCTCTGAACAGGAGTGGGACAAGTAC | | |
| | TGCCACTATGTTGCTGGGCTGGTCGGAATTGGCCTTTCCCGTCTTTTCTCAGCCTCAG | | |
| | AGTTTGAAGACCCCTTAGTTGGTGAAGATACAGAACGTGCCAACTCTATGGGCCTGTT | | |
| | TCTGCAGAAAACAAACATCATCCGTGACTATCTGGAAGACCAGCAAGGAGGAAGAGAG | | |
| | TTCTGGCCTCAAGACGTTTGGAGCAGGTATGTTAAGAAGTTAGGGGATTTTGCTAAGC | | |
| | CGGAGAATATTGACTTGGCCGTGCAGTGCCTGAATGAACTTATAACCAATGCACTGCA | | |
| | CCACATCCCAGATGTCATCACCTACCTTTCGAGACTCAGAAACCAGAGTGTGTTTAAC | | |
| | TTCTGCGCTATTCCACAGGTGATGGCCATTGCCACTTTGGCTGCCTGTTATAATAACC | | |
| | AGCAGGTGTTCAAAGGGGCAGTGAAGATTCGGAAAGGGCAAGCAGTGACCCTGATGAT | | |
| | GGATGCCACCAATATGCCAGCTGTCAAAGCCATCATATATCAGTATATGGAAGAGATT | | |
| | TATCATAGAATCCCCGACTCAGACCCATCTTCTAGCAAAACAAGGCAGATCATCTCCA | | |
| | CCATCCGGACGCAGAATCTTCCCAACTGTCAGCTGATTTCCCGAAGCCACTACTCCCC | | |
| | CATCTACCTGTCGTTTGTCATGCTTTTGGCTGCCCTGAGCTGGCAGTACCTGACCACT | | |
| | CTCTCCCAGGTAACAGAAGACTATGTTCAGACTGGAGAACACTGATCCCAAATTTGTC | | |
| | CATAGCTGAAGTCCACCATAAAGTGGATTTACTTTTTTTCTTTAAAAAAAAAAAAAAA | | |
| | AAAAAAAAAAAAAAA | | |
| | ORF Start: ATG at 10 | | ORF Stop: TGA at 1261 |
| | SEQ ID NO:100 | 417 aa | MW at 48114.8 kD |

| | |
|---|---|
| NOV8a, | MEFVKCLGHPEEFYNLVRFRIGGKRKVMPKMDQDSLSSSLKTCYKYLNQTSRSFAAVI |
| CG119418-01 | |
| Protein Sequence | QALDGEMRNAVCIFYLVLRALDTLEDDMTISVEKKVPLLHNFHSFLYQPDWRFMESKE |
| | KDRQVLEDFPTISLEFRNLAEKYQTVIADICRRMGIGMAEFLDKHVTSEQEWDKYCHY |
| | VAGLVGIGLSRLFSASEFEDPLVGEDTERANSMGLFLQKTNIIRDYLEDQQGGREFWP |
| | QEVWSRYVKKLGDFAKPENIDLAVQCLNELITNALHHIPDVITYLSRLRNQSVFNFCA |
| | IPQVMAIATLAACYNNQQVFKGAVKIRKGQAVTLMMDATNMPAVKAIIYQYMEEIYHR |
| | IPDSDPSSSKTRQIISTIRTQNLPNCQLISRSHYSPIYLSFVMLLAALSWQYLTTLSQ |
| | VTEDYVQTGEH |

Further analysis of the NOV8a protein yielded the following properties shown in Table 8B.

TABLE 8B
Protein Sequence Properties NOV8a

| | |
|---|---|
| PSort | 0.4500 probability located in cytoplasm; |
| analysis: | 0.3719 probability located in microbody |
| | (peroxisome); 0.1000 probability located |
| | in mitochondrial matrix space; 0.1000 |
| | probability located in lysosome (lumen) |
| SignalP | No Known Signal Sequence Predicted |
| analysis: | |

A search of the NOV8a protein against the Geneseq database, a proprietary database that contains sequences published in patents and patent publication, yielded several homologous proteins shown in Table 8C.

TABLE 8C
Geneseq Results for NOV8a

| | | | Identities/ | |
|---|---|---|---|---|
| | | | Similarities for | |
| Geneseq | Protein/Organism/Length | NOV8a Residues/ | the Matched | Expect |
| Identifier | [Patent #, Date] | Match Residues | Region | Value |
| AAW01739 | Human squalene synthetase - | 1 . . . 417 | 417/417 (100%) | 0.0 |
| | Homo sapiens, 417 aa. | 1 . . . 417 | 417/417 (100%) | |
| | [US5589372-A, 31 DEC. 1996] | | | |
| AAR52606 | Human squalene synthase - Homo | 1 . . . 417 | 416/417 (99%) | 0.0 |
| | sapiens, 417 aa. [GB2272442-A, | 1 . . . 417 | 416/417 (99%) | |
| | 18 MAY 1994] | | | |
| ABB57061 | Mouse ischaemic condition related | 1 . . . 413 | 365/413 (88%) | 0.0 |
| | protein sequence SEQ ID NO: 118 - | 1 . . . 413 | 395/413 (95%) | |
| | Mus musculus, 416 aa. | | | |
| | [WO200188188-A2, 22 NOV. | | | |
| | 2001] | | | |
| AAR94574 | Squalene synthetase from Nicotiana | 7 . . . 396 | 177/403 (43%) | 2e−89 |
| | benthamiana - Nicotiana | 8 . . . 401 | 257/403 (62%) | |
| | benthamiana. 411 aa. | | | |
| | [WO9609393-A1, 28 MAR. 1996] | | | |
| AAG32432 | Arabidopsis thaliana protein | 7 . . . 401 | 173/406 (42%) | 8e−88 |
| | fragment SEQ ID NO: 39123 - | 2 . . . 401 | 251/406 (61%) | |
| | Arabidopsis thaliana, 404 aa. | | | |
| | [EP1033405-A2, 06 SEP. 2000] | | | |

In a BLAST search of public sequence datbases, the NOV8a protein was found to have homology to the proteins shown in the BLASTP data in Table 8D.

TABLE 8D
Public BLASTP Results for NOV8a

| | | | Identities/ | |
|---|---|---|---|---|
| Protein | | | Similarities for | |
| Accession | | NOV8a Residues/ | the Matched | Expect |
| Number | Protein/Organism/Length | Match Residues | Portion | Value |
| P37268 | Farnesyl-diphosphate | 1 . . . 417 | 417/417 (100%) | 0.0 |
| | farnesyltransferase (EC 2.5.1.21) | 1 . . . 417 | 417/417 (100%) | |
| | (Squalene synthetase) (SQS) (SS) | | | |
| | (FPP: FPP farnesyltransferase) - | | | |
| | Homo sapiens (Human), 417 aa. | | | |
| Q96GT0 | Farnesyl-diphosphate | 1 . . . 417 | 416/417 (99%) | 0.0 |
| | farnesyltransferase 1 - Homo sapiens | 1 . . . 417 | 417/417 (99%) | |
| | (Human), 417 aa. | | | |
| I38245 | farnesyl-diphosphate | 1 . . . 417 | 416/417 (99%) | 0.0 |
| | farnesyltransferase (EC 2.5.1.21), | 1 . . . 417 | 416/417 (99%) | |
| | hepatic - human, 417 aa. | | | |
| I52090 | squalene synthase - human, 411 aa. | 1 . . . 417 | 415/417 (99%) | 0.0 |
| | | 1 . . . 417 | 417/417 (99%) | |
| P53798 | Farnesyl-diphosphate | 1 . . . 413 | 365/413 (88%) | 0.0 |
| | farnesyltransferase (EC 2.5.1.21) | 1 . . . 413 | 395/413 (95%) | |
| | (Squalene synthetase) (SQS) (SS) | | | |
| | (FPP: FPP farnesyltransferase) - Mus | | | |
| | musculus (Mouse), 416 aa. | | | |

PFam analysis predicts that the NOV8a protein contains the domains shown in the Table 8E.

TABLE 8E
Domain Analysis of NOV8a

| | | Identities/ | |
|---|---|---|---|
| | | Similarities for | |
| Pfam | NOV8a | the Matched | Expect |
| Domain | Match Region | Region | Value |
| SQS_PSY | 47 . . . 334 | 115/317 (36%) | 6.5e−154 |
| | | 280/317 (88%) | |

### Example 9

The NOV9 clone was analyzed, and the nucleotide and encoded polypeptide sequences are shown in Table 9A.

TABLE 9A
NOV9 Sequence Analysis

| | SEQ ID NO:101 | 2106 bp | |
|---|---|---|---|
| NOV9a, | ATGGGGCTTCCTGAGCAGCGGGTCCGGAGCGGCAGCGGGAGCCGGGGCCAGGAGGAAG | | |
| CG120359-01 | | | |
| DNA Sequence | CTGGAGCCGGAGGCCGGGCGCGGAGTTGGTCTCCGCCGCCCGAGGTCAGCCGCTCCGC | | |
| | GCACGTCCCCTCGCTGCAGCGCTACCGCGAGCTGCACCGGCGCTCCGTGGAGGAGCCG | | |
| | CGGGAATTCTGGGGAGACATTGCCAAGGAATTTTACTGGAAGACTCCATGCCCTGGCC | | |
| | CATTCCTTCGGTACAACTTTGATGTGACTAAAGGGAAAATCTTCATTCAGTGGATGAA | | |
| | AGGAGCAACTACCAACATCTGCTACAATGTACTGGATCGAAATGTCCATGAGAAAAAG | | |
| | CTTGGAGATAAAGTTGCTTTTTACTGGGAGGGCAATGAGCCAGGGGAGACCACTCAGA | | |
| | TCACATACCATCAGCTTCTGGTCCAAGTGTGTCAGTTCAGCAATGTTCTCCGAAAACA | | |
| | GGGCATTCAGAAGGGGGACCGAGTGGCCATCTACATGCCTATGATCCCAGAGCTTGTG | | |
| | GTGGCCATGCTGGCATGTGCCCGCATTGGGGCTTTGCACTCCATTGTGTTTGCAGGCT | | |
| | TCTCTTCAGAGTCTCTATGTGAACGGATCTTGGATTCCAGCTGCAGTCTTCTCATCAC | | |
| | TACAGATGCCTTCTACAGGGGGGAAAAGCTTGTGAACCTGAAGGAGCTGGCTGACGAC | | |
| | GCCCTGCAGAAGTGTCAGGAGAAGGGTTTCCCAGTAAGATGCTGCATTGTGGTCAAGC | | |
| | ACCTGGGGCGGGCAGAGCTCGGCATGGGTGACTCCACCAGCCAGTCCCCCCCAATTAA | | |
| | GAGGTCATGCCCAGATGTGCAGATCTCATGGAACCAAGGGATTGACTTGTGGTGGCAT | | |
| | GAGCTCATGCAAGAGGCAGGGGATGAGTGTGAGCCCGAGTGCTGTGATGCCCAGGACC | | |
| | CACTCTTCATCCTGTACACCAGTGGCTCCACAGGCAAACCCAAGGGTGTGGTTCACAC | | |
| | AGTTGGGGGCTACATGCTCTATGTAGCCACAACCTTCAAGTATGTGTTTGACTTCCAT | | |
| | GCAGAGGATGTGTTCTGGTGCACGGCAGACATTGGTTGGATCACTGGTCATTCCTACG | | |
| | TCACCTATGGGCCACTGGCCAATGGTGCCACCAGTGTTTTGTTTGAGGCGATTCCCAC | | |
| | ATATCCGGACGTGAACCGCCTGTGGAGCATTGTGGACAAATACAAGGTGACCAAGTTC | | |
| | TACACAGCACCCACAGCCATCCGTCTGCTCATGAAGTTTGGAGATGAGCCTGTCACCA | | |
| | AGCATAGCCGGGCATCCTTGCAGGTGTTAGGCACAGTGGGTGAACCCATCAACCCTGA | | |
| | GGCCTGGCTATGGTACCACCGGGTGGTAGGTGCCCAGCGCTGCCCCATCGTGGACACC | | |
| | TTCTGGCAAACAGAGACAGGTGGCCACATGTTGACTCCCCTTCCTGGTGCCACACCCA | | |
| | TGAAACCCGGTTCTGCTACTTTCCCATTCTTTGGTGTAGCTCCTGCAATCCTGAATGA | | |
| | GTCCGGGGAAGAGTTGGAAGGTGAAGCTGAAGGTTATCTGGTGTTCAAGCAGCCCTGG | | |
| | CCAGGGATCATGCGCACAGTCTATGGGAACCACGAACGCTTTGAGACAACCTACTTTA | | |
| | AGAAGTTTCCTGGATACTATGTTACAGGAGATGGCTGCCAGCGGGACCAGGATGGCTA | | |
| | TTACTGGATCACTGGCAGGATTGATGACATGCTCAATGTATCTGGACACCTGCTGAGT | | |
| | ACAGCAGACGTGGAGTCAGCACTTGTGGAACATGAGGCTGTTGCAGAGGCAGCTGTGG | | |
| | TGGGCCACCCTCATCCTGTGAAGGGTGAATGCCTCTACTGCTTTGTCACCTTGTGTGA | | |
| | TGGCCACACCTTCAGCCCCAAGCTCACCGAGGAGCTCAAGAAGCAGATTAGAGAAAAG | | |
| | ATTGGCCCCATTGCCACACCAGACTACATCCAGAATGCACCTGGCTTGCCTAAAACCC | | |
| | GCTCAGGGAAAATCATGAGGCGAGTGCTTCGGAACATTGCTCAGAATGACCATGACCT | | |
| | CGGGGACATGTCTACTGTGGCTGACCCATCTGTCATCAGTCACCTCTTCAGCCACCGC | | |
| | TGCCTGACCATCCAGTGA | | |
| | ORF Start: ATG at 1 | | ORF Stop: TGA at 2104 |
| | SEQ ID NO:102 | 701 aa | MW at 78578.9 kD |
| NOV9a, | MGLPEERVRSGSGSRGQEEAGAGGRARSWSPPPEVSRSAHVPSLQRYRELHRRSVEEP | | |
| CG120359-01 | | | |
| Protein Sequence | REFWGDIAKEFYWKTPCPGPFLRYNFDVTKGKIFIEWMKGATTNICYNVLDRNVHEKK | | |
| | LGDKVAFYWEGNEPGETTQITYHQLLVQVCQFSNVLRKQGIQKGDRVAIYMPMIPELV | | |
| | VANLACARIGALHSIVFAGFSSESLCERILDSSCSLLITTDAFYRGEKLVNLKELADE | | |
| | ALQKCQEKGFPVRCCIVVKHLGRAELGMGDSTSQSPPIKRSCPDVQISWNQGIDLWWH | | |
| | ELMQEAGDECEPEWCDAEDPLFILYTSGSTGKPKGVVHTVGGYMLYVATTFKYVFDFH | | |
| | AEDVFWCTADIGWITGHSYVTYGPLANGATSVLFEGIPTYPDVNRLWSIVDKYKVTKE | | |
| | YTAPTAIRLLMKFGDEPVTKHSRASLQVLGTVGEPINPEAWLWYHRVVGAQRCPIVDT | | |
| | FWQTETGGHMLTPLPGATPMKPGSATFPFFGVAPAILNESGEELEGEAEGYLVFKQPW | | |
| | PGIMRTVYGNHERFETTYFKKFPGYYVTGDGCQRDQDGYYWITGRIDDMLNVSGHLLS | | |
| | TAEVESALVEHEAVAEAAVVGHPHPVKGECLYCFVTLCDCHTFSPKLTEELKKQIREK | | |
| | IGPIATPDYIQNAPGLPKTRSGKIMRRVLRKIAQNDHDLGDMSTVADPSVISHLFSHR | | |
| | CLTIQ | | |
| | SEQ ID NO:103 | 2125 bp | |
| NOV9b, | CACCGGATCCACCATGGGGCTTCCTGAGGAGCGGGTCCGGAGCGGCAGCGGGAGCCGG | | |
| 277685717 DNA | | | |
| Sequence | GGCCAGGAGGAAGCTGGAGCCGGAGGCCGGGCGCGGAGTTGGTCTCCGCCGCCCGAGG | | |
| | TCAGCCGCTCCGCGCACGTCCCCTCGCTGCAGCGCTACCGCGAGCTGCACCGGCGCTC | | |
| | CGTGGAGGAGCCGCGGGAATTCTGGGGAGACATTGCCAAGGAATTTTACTGGAAGACT | | |
| | CCATGCCCTGGCCCATTCCTTCGGTACAACTTTGATGTGACTAAAGGCAAAATCTTTA | | |
| | TTGAGTGGATGAAAGGAGCAACTACCAACATCTGCTACAATGTACTGGATCGAAATGT | | |
| | CCATGAGAAAAAGCTTGGAGATAAAGTTGCTTTTTACTGGGAGGGCAATGAGCCAGGG | | |
| | GAGACCACTCAGATCACATACCATCAGCTTCTGGTCCAAGTGTGTCAGTTCAGCAATG | | |
| | TTCTCCGAAAACAGGGCATTCAGAAGGGGGACCGAGTGGCCATCTACATGCCTATGAT | | |
| | CCCAGAGCTTGTGGTGGCCATGCTGGCATGTGCCCGCATTGGGGCTTTGCACTCCATT | | |
| | GTGTTTGCAGGCTTCTCTTCAGAGTCTCTATGTGAACGGATCTTGGATTCCAGCTGCA | | |
| | GTCTTCTCATCACTACAGATGCCTTCTACAGGGGGGAAAAGCTTGTGAACCTGAAGGA | | |
| | GCTGGCTGACGAGGCCCTGCAGAAGTGTCAGGACAAGGGTTTCCCAGTAAGATGCTGC | | |
| | ATTGTGGTCAAGCACCTGGGGCGGGCAGAGCTCGGCATGGGTGACTCCACCAGCCAGT | | |
| | CCCCCCCAATTAAGAGGTCATGCCCAGATGTGCAGATCTCATGGAACCAAGGGATTGA | | |
| | CTTCTGGTGGCATGAGCTCATGCAAGAGGCAGGGGATGAGTGTGAGCCCGAGTGGTGT | | |
| | GATGCCGAGGACCCACTCTTCATCCTGTACACCAGTGGCTCCACAGGCAAACCCAAGG | | |
| | GTGTGGTTCACACAGTTGGGCGCTACATGCTCTATGTAGCCACAACCTTCAAGTATGT | | |
| | GTTTGACTTCCATCCAGAGGATGTGTTCTGGTGCACGGCAGACATTGGTTGGATCACT | | |
| | GGTCATTCCTACGTCACCTATGGGCCACTGGCCAATGGTGCCACCAGTCTTTTGTTTG | | |
| | ACGGGATTCCCACATATCCGGACGTGAACCGCCTGTGGAGCATTGTGGACAAATACAA | | |
| | GGTGACCAAGTTCTACACAGCACCCACAGCCATCCGTCTGCTCATGAAGTTTGGAGAT | | |
| | GAGCCTGTCACCAAGCATAGCCGGGCATCCTTGCAGGTGTTAGGCACAGTGGGTGAAC | | |
| | CCATCAACCCTGAGGCCTGGCTATGGTACCACCGGGTGGTAGGTGCCCAGCGCTGCCC | | |
| | CATCGTGGACACCTTCTGGCAAACAGAGACAGGTGGCCACATGTTGACTCCCCTTCCT | | |
| | GGTGCCACACCCATGAAACCCGGTTCTGCTACTTTCCCATTCTTTGGTGTAGCTCCTG | | |
| | CAATCCTGAATGAGTCCGGGGAAGAGTTGGAAGGTGAAGCTGAAGGTTATCTGGTGTT | | |
| | CAAGCAGCCCTGGCCAGGGATCATGCGCACAGTCTATGGGAACCACGAACGCTTTGAG | | |
| | ACAACCTACTTTAAGAAGTTTCCTGGATACTATGTTACAGGAGATGGCTGCCAGCGGG | | |
| | ACCAGGATGGCTATTACTGGATCACTGGCAGGATTGATGACATGCTCAATGTATCTGG | | |
| | ACACCTGCTGAGTACAGCAGAGGTGGAGTCAGCACTTGTGGAACATGAGGCTGTTGCA | | |
| | GAGGCAGCTGTGGTGGGCCACCCTCATCCTGTGAAGGGTGAATGCCTCTACTGCTTTG | | |
| | TCACCTTGTGTGATGGCCACACCTTCAGCCCCAAGCTCACCGAGGAGCTCAAGAAGCA | | |
| | GATTAGAGAAAAGATTGGCCCCATTGCCACACCAGACTACATCCAGAATGCACCTGGC | | |
| | TTGCCTAAAACCCGCTCAGGGAAAATCATGAGGCGAGTGCTTCGGAAGATTGCTCAGA | | |
| | ATGACCATGACCTCGGGGACATGTCTACTGTGGCTCACCCATCTGTCATCAGTCACCT | | |
| | CTTCAGCCACCGCTGCCTGACCATCCAGCTCGAGGGC | | |
| | ORF Start: at 2 | | ORF Stop: end of |
| | | | sequence |
| | SEQ ID NO:104 | 708 aa | MW at 79224.6 kD |
| NOV9b, | TGSTMGLPEERVRSGSGSRGQEEAGAGGRARSWSPPPEVSRSAHVPSLQRYRELHRRS | | |
| 277685717 | | | |
| Protein Sequence | VEEPREFWCDIAKEFYWKTPCPGPFLRYNFDVTKGKIFIEWNKGATTNICYNVLDRNV | | |
| | HEKKLCDKVAFYWEGNEPGETTQITYHQLLVQVCQFSNVLRKQGIQKGDRVAIYMPMI | | |
| | PELVVAMLACARIGALHSIVFAGFSSESLCERILDSSCSLLITTDAFYRGEKLVMLKE | | |
| | LADEALQKCQEKGFPVRCCIVVKHLGRAELGMGDSTSQSPPIKRSCPDVQISWNQGID | | |
| | LWWHELMQEAGDECEPEWCDAEDPLFILYTSGSTGKPKGVVHTVGGYMLYVATTFKYV | | |
| | FDFHAEDVFWCTADIGWITGHSYVTYGPLANGATSVLFEGIPTYPDVNRLWSIVDKYK | | |
| | VTKFYTAPTAIRLLMKFGDEPVTKHSRASLQVLGTVGEPINPEAWLWYHRVVGAQRCP | | |
| | IVDTFWQTETGGHMLTPLPGATPMKPGSATFPFFGVAPAILNESGEELEGEAEGYLVF | | |
| | KQPWPGIMRTVYGNHERFETTYFKKFPGYYVTGDGCQRDQDGYYWITGRIDDMLNVSG | | |
| | HLLSTAEVESALVEHEAVAEAAVVGHPHPVKGECLYCFVTLCDGHTFSPKLTEELKKQ | | |
| | IREKIGPIATPDYIQNAPGLPKTRSGKIMRRVLRKIAQNDHDLGDMSTVADPSVISHL | | |
| | FSHRCLTIQLEG | | |
| | SEQ ID NO:105 | 1408 bp | |
| NOV9c, | CACCGGATCCACATACCATCAGCTTCTGGTCCAAGTGTGTCAGTTCAGCAATGTTCTC | | |
| 277686882 DNA | | | |
| Sequence | CGAAAACAGGGCATTCAGAAGGGGGACCGAGTGGCCATCTACATGCCTATGATCCCAG | | |
| | AGCTTGTGGTGGCCATGCTGGCATGTGCCCGCATTGGGGCTTTGCACTCCATTGTGTT | | |
| | TGCAGGCTTCTCTTCAGAGTCTCTATGTGAACGGATCTTGCATTCCAGCTGCAGTCTT | | |
| | CTCATCACTACAGATGCCTTCTACAGGGGGGAAAAGCTTGTGAACCTGAAGGAGCTGG | | |
| | CTGACGAGGCCCTGCAGAAGTGTCAGGAGAAGGGTTTCCCAGTAAGATGCTGCATTGT | | |
| | GGTCAAGCACCTGGGGCGGGCAGAGCTCGGCATGGGTGACTCCACCAGCCAGTCCCCC | | |
| | CCAATTAAGAGGTCATGCCCAGATGTGCAGATCTCATGGAACCAAGGGATTGACTTGT | | |
| | GGTGGCATGACCTCATGCAAGAGGCAGGGGATGAGTGTGAGCCCGAGTGGTGTGATGC | | |
| | CGAGGACCCACTCTTCATCCTGTACACCAGTGGCTCCACAGGCAAACCCAAGGGTGTG | | |
| | GTTCACACAGTTGGGGGCTACATGCTCTATGTAGCCACAACCTTCAAGTATGTGTTTG | | |
| | ACTTCCATGCAGAGGATGTGTTCTGGTGCACGGCAGACATTGGTTGGATCACTGGTCA | | |
| | TTCCTACGTCACCTATGGGCCACTGGCCAATGGTGCCACCAGTGTTTTGTTTGAGGGG | | |
| | ATTCCCACATATCCGGACGTGAACCGCCTGTGGAGCATTGTGGACAAATACAAGGTGA | | |
| | CCAAGTTCTACACAGCACCCACAGCCATCCGTCTGCTCATGAAGTTTGGAGATGAGCC | | |
| | TGTCACCAAGCATAGCCGGGCATCCTTGCAGGTGTTAGGCACAGTGGGTGAACCCATC | | |
| | AACCCTGAGGCCTGGCTATGGTACCACCGGGTGGTAGGTGCCCAGCGCTGCCCCATCG | | |
| | TGGACACCTTCTGGCAAACAGAGACAGGTGGCCACATGTTGACTCCCCTTCCTGGTGC | | |
| | CACACCCATGAAACCCGGTTCTGCTACTTTCCCATTCTTTGGTGTAGCTCCTGCAATC | | |
| | CTGAATGAGTCCGGGGAAGAGTTGGAAGGTGAAGCTGAAGGTTATCTGGTGTTCAAGC | | |
| | AGCCCTGGCCAGGGATCATGCGCACAGTCTATGGGAACCACGAACGCTTTGAGACAAC | | |
| | CTACTTTAAGAAGTTTCCTGGATACTATGTTACAGGAGATGGCTGCCAGCGGGACCAG | | |
| | GATCGCTATTACTGGATCACTGGCAGGATTGATGACATGCTCAATGTATCTGGACACC | | |
| | TGCTGAGTACAGCAGAGGTGGAGTCAGCACTTGTGGAACATGAGGCTGTTGCAGAGGC | | |
| | AGCTGTGCTCGAGGGC | | |
| | ORF Start: at 2 | | ORF Stop: end of |
| | | | sequence |
| | SEQ ID NO:106 | 469 aa | MW at 52125.0 kD |
| NOV9c, | TGSTYHQLLVQVCQFSNVLRKQGIQKGDRVAIYMPMTPELVVAMLACARIGALHSIVF | | |
| 277686882 | | | |
| Protein Sequence | AGFSSESLCERILDSSCSLLITTDAFYRGEKLVNLKELADEALQKCQEKGPPVRCCIV | | |
| | VKHLGRAELGMGDSTSQSPPTKRSCPDVQISNNQGIDLWWHELMQEAGDECEPEWCDA | | |
| | EDPLFILYTSGSTGKPKGVVHTVGGYMLYVATTFKYVFDFHAEDVFWCTADIGWITGH | | |
| | SYVTYGPLANGATSVLFEGIPTYPDVNRLWSIVDKYKVTKFYTAPTAIRLLMKFGDEP | | |
| | VTKHSRASLQVLGTVGEPINPEAWLWYHRVVGAQRCPIVDTFWQTETGGHMLTPLPGA | | |
| | TPMKPGSATFPFFGVAPAILNESGEELEGEAEGYLVFKQPWPGIMRTVYGNHERFETT | | |
| | YFKKFPGYYVTGDGCQRDQDGYYWITGRIDDMLNVSGHLLSTAEVESALVEHEAVAEA | | |
| | AVLEG | | |
| | SEQ ID NO:107 | 2164 bp | |
| NOV9d, | CACCGGATCCACCATGGGGCTTCCTGAGCAGCGCGTCCGGAGCGGCAGCGGGAGCCGG | | |
| CG120359-02 | | | |
| DNA Sequence | GGCCAGGAGGAAGCTGGAGCCGGAGGCCGGGCGCGGAGTTGGTCTCCGCCGCCCGAGG | | |
| | TCAGCCGCTCCGCGCACGTCCCCTCGCTGCAGCGCTACCGCGAGCTGCACCGGCGCTC | | |
| | CGTGGAGGAGCCGCGGGAATTCTGGGGAGACATTGCCAAGGAATTTTACTGGAAGACT | | |
| | CCATGCCCTGGCCCATTCCTTCGGTACAACTTTGATGTGACTAAAGGGAAAATCTTCA | | |
| | TTGAGTGGATGAAAGGAGCAACTACCAACATCTGCTACAATGTACTGGATCGAAATGT | | |
| | CCATGAGAAAAAGCTTGGAGATAAAGTTGCTTTTTACTGGTCCACTTCTGGTAATTCA | | |
| | TCCTACAGATATACTTGCAGGGAGGGCAATGAGCCAGGGGAGACCACTCAGATCACAT | | |
| | ACCATCAGCTTCTGGTCCAAGTGTGTCAGTTCAGCAATGTTCTCCGAAAACAGGGCAT | | |
| | TCAGAAGGGGGACCGAGTGGCCATCTACATGCCTATGATCCCAGAGCTTGTGGTGGCC | | |
| | ATCCTGGCATGTGCCCGCATTGGGGCTTTGCACTCCATTGTGTTTGCAGGCTTCTCTT | | |
| | CAGAGTCTCTATGTGAACGGATCTTCGATTCCAGCTGCAGTCTTCTCATCACTACAGA | | |
| | TGCCTTCTACAGGGGGGAAAAGCTTGTGAACCTGAAGGAGCTGGCTGACGAGGCCCTG | | |
| | CAGAAGTGTCAGGAGAAGGGTTTCCCAGTAAGATGCTGCATTGTGGTCAAGCACCTGG | | |
| | GGCGGGCAGAGCTCGGCATGGGTGACTCCACCAGCCAGTCCCCCCCAATTAAGAGGTC | | |
| | ATGCCCAGATGTGCAGATCTCATGGAACCAAGGGATTGACTTGTGGTGGCATGAGCTC | | |
| | ATGCAAGAGGCAGGGGATGAGTGTGAGCCCGAGTGGTGTGATGCCGAGGACCCACTCT | | |
| | TCATCCTGTACACCAGTGGCTCCACAGGCAAACCCAAGGGTGTGGTTCACACAGTTGG | | |
| | GGGCTACATGCTCTATCTAGCCACAACCTTCAAGTATGTGTTTGACTTCCATGCAGAG | | |
| | GATGTGTTCTGGTGCACGGCAGACATTGGTTGGATCACTGGTCATTCCTACGTCACCT | | |
| | ATGGGCCACTGGCCAATGGTGCCACCAGTGTTTTGTTTGAGGGGATTCCCACATATCC | | |
| | GGACGTGAACCGCCTGTGGAGCATTGTGGACAAATACAAGGTGACCAAGTTCTACACA | | |
| | GCACCCACAGCCATCCGTCTGCTCATGAAGTTTGGAGATGAGCCTGTCACCAAGCATA | | |
| | GCCGGGCATCCTTCCAGGTGTTAGGCACAGTGGGTGAACCCATCAACCCTGAGGCCTG | | |
| | GCTATGGTACCACCGGGTGGTAGGTGCCCAGCGCTGCCCCATCGTGGACACCTTCTGG | | |
| | CAAACAGAGACAGGTGGCCACATGTTGACTCCCCTTCCTGGTGCCACACCCATGAAAC | | |
| | CCGGTTCTGCTACTTTCCCATTCTTTGGTGTAGCTCCTGCAATCCTGAATGAGTCCGG | | |
| | GGAAGAGTTGGAAGGTGAAGCTGAAGGTTATCTGGTGTTCAAGCAGCCCTGGCCAGGG | | |
| | ATCATGCGCACAGTCTATGGGAACCACGAACGCTTTGAGACAACCTACTTTAAGAAGT | | |
| | TTCCTGGATACTATGTTACAGGAGATGGCTGCCAGCGGCACCAGGATGGCTATTACTG | | |
| | GATCACTGGCAGGATTGATGACATGCTCAATGTATCTGGACACCTGCTGAGTACAGCA | | |
| | GAGGTGGAGTCAGCACTTGTGGAACATGAGGCTGTTGCAGAGGCAGCTGTGGTGGGCC | | |
| | ACCCTCATCCTGTGAAGGGTGAATGCCTCTACTGCTTTGTCACCTTGTGTGATGGCCA | | |
| | CACCTTCAGCCCCAAGCTCACCGAGGAGCTCAAGAAGCAGATTAGAGAAAAGATTGGC | | |
| | CCCATTGCCACACCAGACTACATCCAGAATGCACCTGGCTTGCCTAAAACCCGCTCAG | | |
| | GGAAAATCATGAGGCGAGTGCTTCGGAAGATTGCTCAGAATGACCATGACCTCGGGGA | | |
| | CATGTCTACTGTGGCTGACCCATCTGTCATCAGTCACCTCTTCAGCCACCGCTGCCTG | | |
| | ACCATCCAGCTCGAGGGC | | |
| | ORF Start: ATG at 14 | | ORF Stop: at 2156 |
| | SEQ ID NO:108 | 714 aa | MW at 80042.4 kD |

| | |
|---|---|
| NOV9d, | MGLPEERVRSGSGSRGQEEAGAGGRARSWSPPPEVSRSAHVPSLQRYRELHRRSVEEP |
| CG120359-02 | |
| Protein Sequence | REFWGDIAKEFYWKTPCPGPFLRYNFDVTKGKIFIEWMKGATTNICYNVLDRNVHEKK |
| | LGDKVAFYWSTSGNSSYRYTCREGNEPGETTQITYHQLLVQVCQFSNVLRKQGIQKGD |
| | RVAIYMPMIPELVVAMLACARIGALHSIVFAGFSSESLCERILDSSCSLLITTDAFYR |
| | GEKLVNLKELADEALQKCQEKGFPVRCCIVVKHLGRAELGMGDSTSQSPPIKRSCPDV |
| | QISWNQGIDLWWHELMQEAGDECEPEWCDAEDPLFILYTSGSTGKPKGVVHTVGGYML |
| | YVATTFKYVFDFHAEDVFWCTADIGWITGHSYVTYGPLANGATSVLFEGIPTYPDVNR |
| | LWSIVDKYKVTKFYTAPTAIRLLMKFGDEPVTKHSRASLQVLGTVGEPINPEAWLWYH |
| | RVVGAQRCPIVDTFWQTETGGHMLTPLPGATPMKPGSATFPFFGVAPAILNESGEELE |
| | GEAEGYLVFKQPWPGIMRTVYGNHERFETTYFKKFPGYYVTGDCCQRDQDGYYWITGR |
| | IDDMLNVSGHLLSTAEVESALVEHEAVAEAAVVGHPHPVKGECLYCFVTLCDGHTFSP |
| | KLTEELKKQIREKIGPIATPDYIQNAPGLPKTRSGKIMRRVLRKIAQNDHDLGDMSTV |
| | ADPSVISHLFSHRCLTIQ |

Sequence comparison of the above protein sequences yields the following sequence relationships shown in Table 9B.

TABLE 9B
Comparison of NOV9a against NOV9b through NOV9d.

| | | Identities/ |
|---|---|---|
| | | Similarities for |
| Protein | NOV9a Residues/ | the Matched |
| Sequence | Match Residues | Region |
| NOV9b | 1 . . . 701 | 701/701 (100%) |
| | 5 . . . 705 | 701/701 (100%) |
| NOV9c | 134 . . . 600 | 464/467 (99%) |
| | 1 . . . 467 | 465/467 (99%) |
| NOV9d | 1 . . . 701 | 701/714 (98%) |
| | 1 . . . 714 | 701/714 (98%) |

Further analysis of the NOV9a protein yielded the following properties shown in Table 9C.

TABLE 9C
Protein Sequence Properties NOV9a

| | |
|---|---|
| PSort | 0.9000 probability located in Golgi body; |
| analysis: | 0.7900 probability located in plasma |
| | membrane; 0.7166 probability located in |
| | microbody (peroxisome); 0.2000 probability |
| | located in endoplasmic reticulum (membrane) |
| SignalP | No Known Signal Sequence Predicted |
| analysis: | |

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identities/ | |
|---|---|---|---|---|
| | | Residues/ | Similarities | |
| Geneseq | Protein/Organism/Length [Patent | Match | for the Matched | Expect |
| Identifier | #, Date] | Residues | Region | Value |
| AAM41491 | Human polypeptide SEQ ID NO | 59 . . . 701 | 641/643 (99%) | 0.0 |
| | 6422 - Homo sapiens, 651 aa. | 9 . . . 651 | 642/643 (99%) | |
| | [WO200153312-A1, 26 JUL. 2001] | | | |
| AAM39705 | Human polypeptide SEQ ID NO | 60 . . . 701 | 641/642 (99%) | 0.0 |
| | 2850 - Homo sapiens, 666 aa. | 25 . . . 666 | 641/642 (99%) | |
| | [WO200153312-A1, 26 JUL. 2001] | | | |
| AAB42913 | Human ORFX ORF2677 | 96 . . . 701 | 593/606 (97%) | 0.0 |
| | polypeptide sequence SEQ ID | 1 . . . 605 | 594/606 (97%) | |
| | NO:5354 - Homo sapiens, 605 aa. | | | |
| | [WO200058473-A2, 05 OCT. 2000] | | | |
| AAB94113 | Human protein sequence SEQ ID | 260 . . . 701 | 441/442 (99%) | 0.0 |
| | NO: 14352 - Homo sapiens, 442 aa. | 1 . . . 442 | 442/442 (99%) | |
| | [EP1074617-A2, 07 FEB. 2001] | | | |
| ABB71619 | Drosophila melanogaster | 29 . . . 696 | 420/670 (62%) | 0.0 |
| | polypeptide SEQ ID NO 41649 - | 8 . . . 665 | 522/670 (77%) | |
| | Drosophila melanogaster, 670 aa. | | | |
| | [WO200171042-A2, 27 SEP. 2001] | | | |

In a BLAST search of public sequence datbases, the NOV9a protein was found to have homology to the proteins shown in the BLASTP data in Table 9E.

TABLE 9E
Public BLASTP Results for NOV9a

| | | NOV9a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9NR19 | Acetyl-coenzyme A synthetase, | 1 . . . 701 | 701/701 (100%) | 0.0 |
| | cytoplasmic (EC. 6.2.1.1)(Acetate-- | 1 . . . 701 | 701/701 (100%) | |
| | CoA ligase) (Acyl-activating | | | |
| | enzyme) (Acetyl-CoA synthetase) | | | |
| | (ACS) (AceCS) - Homo sapiens | | | |
| | (Human), 701 aa. | | | |
| BAC03849 | CDNA FLJ34962 fis, clone | 1 . . . 701 | 699/714 (97%) | 0.0 |
| | NTONG2003897, highly similar to | 1 . . . 714 | 700/714 (97%) | |
| | Homo sapiens acetyl-CoA | | | |
| | synthetase mRNA - Homo sapiens | | | |
| | (Human), 714 aa. | | | |
| BAC04235 | CDNA fis, clone TRACH2001275, | 1 . . . 701 | 653/701 (93%) | 0.0 |
| | highly similar to Mus musculus | 1 . . . 701 | 676/701 (96%) | |
| | acetyl-CoA synthetase mRNA - Mus | | | |
| | musculus (Mouse), 701 aa. | | | |
| Q9QXG4 | Acetyl-coenzyme A synthetase, | 1 . . . 701 | 651/701 (92%) | 0.0 |
| | cytoplasmic (EC 6.2.1.1) (Acetate- | 1 . . . 701 | 673/701 (95%) | |
| | CoA ligase) (Acyl-activating | | | |
| | enzyme) (Acetyl-CoA synthetase) | | | |
| | (ACS) (AceCS) - Mus musculus | | | |
| | (Mouse), 701 aa. | | | |
| Q96FY7 | Unknown (protein for MGC: 19474) - | 260 . . . 701 | 442/442 (100%) | 0.0 |
| | Homo sapiens (Human), 442 aa. | 1 . . . 442 | 442/442 (100%) | |

PFam analysis predicts that the NOV9a protein contains the domains shown in the Table 9F.

TABLE 9F
Domain Analysis of NOV9a

| | NOV9a | Identities/Similarities | Expect |
|---|---|---|---|
| Pfam Domain | Match Region | for the Matched Region | Value |
| AMP-binding | 137 . . . 599 | 125/465 (27%) | 2.4e−127 |
| | | 354/465 (76%) | |

### Example 10

The NOV10 clone was analyzed, and the nucleotide and encoded polypeptide sequences are shown in Table 10A.

TABLE 10A
NOV10 Sequence Analysis

| | SEQ ID NO:109 | 1958 bp | |
|---|---|---|---|
| NOV10a, | GCAGGCCAGCCCCATGGGGAAGCGCAGACGCCGGNGCCTGGGCGCTCTGAGATTGTCA | | |
| CG124907-01 | | | |
| DNA Sequence | CTGCTGTTCCAAGGGCACACGCAGAGGGATTTGGAATTCCTGGAGAGTTGCCTTTGTG | | |
| | AGAAGCTGGAAATATTTCTTTCAATTCCATCTCTTAGTTTTCCATAGGAACATCAAGA | | |
| | AATCATGAACAACTTTGGTAATGAAGAGTTTGACTGCCACTTCCTCGATGAAGGTTTT | | |
| | ACTGCCAAGGACATTCTGGACCAGAAAATTAATGAAGTTTCTTCTTCTGATGATAAGG | | |
| | ATGCCTTCTATGTGGCAGACCTGGGAGACATTCTAAAGAAACATCTGAGGTGGTTAAA | | |
| | AGCTCTCCCTCGTGTCACCCCCTTTTATGCAGTCAAATGTAATGATAGCAAAGCCATC | | |
| | GTGAAGACCCTTGCTGCTACCGGGACAGGATTTGACTGTGCTAGCAAGACTGAAATAC | | |
| | AGTTGGTGCAGAGTCTGGGGGTGCCTCCAGAGAGGATTATCTATGCAAATCCTTGTAA | | |
| | ACAAGTATCTCAAATTAAGTATGCTGCTAATAATGGAGTCCAGATGATGACTTTTGAT | | |
| | AGTGAAGTTGAGTTGATGAAAGTTGCCAGAGCACATCCCAAAGCAAAGTTGGTTTTGC | | |
| | GGATTGCCACTGATGATTCCAAAGCAGTCTGTCGTCTCAGTGTCAAATTCGGTGccAc | | |
| | GCTCAGAACCAGCAGGCTCCTTTTGGAACGGGCGAAAGAGCTAAATATCGATGTTGTT | | |
| | GGTGTCAGCTTCCATGTAGGAAGCGGCTGTACCGATCCTGAGACCTTCGTGCAGGCAA | | |
| | TCTCTGATGCCCGCTGTGTTTTTGACATGGGGGCTGAGGTTGGTTTCAGCATGTACT | | |
| | GCTTGATATTGGCCGTGGCTTTCCTGGATCTGAGGATGTGAAACTTAAATTTGAAGAG | | |
| | ATCACCGGCGTAATCAACCCAGCGTTGGACAAATACTTTCCGTCAGACTCTGGAGTGA | | |
| | GAATCATAGCTGAGCCCGGCAGATACTATGTTGCATCAGCTTTCACGCTTGCAGTTAA | | |
| | TATCATTGCCAAGAAAATTGTATTAAAGGAACAGACGGGCTCTGATGACGAAGATGAG | | |
| | TCGAGTGAGCAGACCTTTATGTATTATGTGAATGATGGCGTCTATGGATCATTTAATT | | |
| | GCATACTCTATGACCACGCACATGTAAAGCCCCTTCTGCAAAAGAGACCTAAACCAGA | | |
| | TGAGAAGTATTATTCATCCAGCATATGGGGACCAACATGTGATGGCCTCGATCGGATT | | |
| | GTTGAGCGCTGTCACCTGCCTGAAATGCATGTGGGTGATTGGATGCTCTTTGAAAACA | | |
| | TGGGCGCTTACACTGTTGCTGCTGCCTCTACGTTCAATGGCTTCCAGAGGCCGACGAT | | |
| | CTACTATGTGATGTCAGGGCCTGCGTGGCAACTCATGCAGCAATTCCAGAACCCCGAC | | |
| | TTCCCACCCGAAGTAGAGGAACAGGATGCCAGCACCCTGCCTGTGTCTTGTGCCTGGG | | |
| | AGAGTGGGATGAAACGCCACAGAGCAGCCTGTGCTTCGGCTAGTATTAATGTGTAGAT | | |
| | AGCACTCTGGTAGCTGTTAACTGCAAGTTTAGCTTGAATTAAGGGATTTGGGGGGACC | | |
| | ATGTAACTTAATTACTGCTAGTTTTGAAATGTCTTTGTAAGAGTAGGGTCGCCATGAT | | |
| | GCAGCCATATGGAAGACTAGGATATGGGTCACACTTATCTGTGTTCCTATGCAAACTA | | |
| | TTTGAATATTTGTTTTATATGGATTTTTATTCACTCTTCAGACACGCTACTCAAGAGT | | |
| | GCCCCTCAGCTGCTGAACAAGCATTTGTAGCTTGTACAATGGCAGAATGGGCCAAAAG | | |
| | CTTAGTGTTGTGACCTGTTTTTAAAATAAAGTATCTTGAAATAAACAAAAAAAAAAAA | | |
| | GGGGGGCCGCCCTAGGGGTTCCCAAGTTTACGTACGCTGCATGG | | |
| | ORF Start: ATG at 179 | | ORF Stop: TAG at 1562 |
| | SEQ ID NO:110 | 461 aa | MW at 51147.6 kD |
| NOV10a, | MNNFGNEEFDCHFLDEGFTAKDILDQKINEVSSSDDKDAFYVADLGDILKKHLRWLKA | | |
| CG124907-01 | | | |
| Protein Sequence | LPRVTPFYAVKCNDSKAIVKTLAATGTGFDCASKTEIQLVQSLGVPPERIIYANPCKQ | | |
| | VSQIKYAANNGVQMMTFDSEVELMKVARAHPKAKLVLRIATDDSKAVCRLSVKFGATL | | |
| | RTSRLLLERAKELNIDVVGVSFHVGSGCTDPETFVQAISDARCVFDMGAEVGFSMYLL | | |
| | DTGGGFPGSEDVKLKFEEITGVINPALDKYFPSDSGVRIIAEPGRYYVASAFTLAVNI | | |
| | IAKKIVLKEQTGSDDEDESSEQTFMYYVNDGVYGSFNCILYDHAHVKPLLQKRPKPDE | | |
| | KYYSSSIWGPTCDGLDRIVERCDLPEMHVGDWMLFENMGAYTVAAASTFNGFQRPTIY | | |
| | YVMSGPAWQLMQQFQNPDFPPEVEEQDASTLPVSCAWESGMKRHRAACASASINV | | |
| | SEQ ID NO:111 | 1958 bp | |
| NOV10b, | GCAGGCCAGCCCCATGGGGAAGCGCAGACGCCGGNGCCTGGGCGCTCTGAGATTGTCA | | |
| CG124907-01 | | | |
| DNA Sequence | CTGCTGTTCCAAGGGCACACGCAGAGGGATTTGGAATTCCTGGAGAGTTGCCTTTGTG | | |
| | AGAAGCTGGAAATATTTCTTTCAATTCCATCTCTTAGTTTTCCATAGGAACATCAAGA | | |
| | AATCEE ATGAACAACTTTGGTAATGAAGAGTTTGACTGCCACTTCCTCGATGAAGGTTTT | | |
| | ACTGCCAAGGACATTCTGGACCAGAAAATTAATGAAGTTTCTTCTTCTGATGATAAGG | | |
| | ATGCCTTCTATGTGGCAGACCTGGGAGACATTCTAAAGAAACATCTGAGGTGGTTAAA | | |
| | AGCTCTCCCTCGTGTCACCCCCTTTTATGCAGTCAAATGTAATGATAGCAAAGCCATC | | |
| | GTGAAGACCCTTGCTGCTACCGGGACAGGATTTGACTGTGCTAGCAAGACTGAAATAC | | |
| | AGTTGGTGCACAGTCTGGGGGTGCCTCCAGAGAGGATTATCTATGCAAATCCTTGTAA | | |
| | ACAAGTATCTCAAATTAAGTATGCTGCTAATAATGGAGTCCAGATGATGACTTTTGAT | | |
| | AGTGAAGTTGAGTTGATGAAAGTTGCCAGAGCACATCCCAAAGCAAAGTTGGTTTTGC | | |
| | CGATTGCCACTGATGATTCCAAAGCAGTCTGTCGTCTCAGTGTGAAATTCGGTGCCAC | | |
| | GCTCAGAACCAGCAGCCTCCTTTTGGAACGGGCGAAAGAGCTAAATATCGATGTTGTT | | |
| | GGTGTCAGCTTCCATGTAGGAAGCGGCTGTACCGATCCTGAGACCTTCGTGCAGGCAA | | |
| | TCTCTGATGCCCGCTGTGTTTTTGACATGGGGGCTGAGGTTGGTTTCAGCATGTATCT | | |
| | GCTTGATATTGGCGGTGGCTTTCCTCGATCTGAGGATGTGAAACTTAAATTTGAAGAG | | |
| | ATCACCGGCGTAATCAACCCAGCGTTGGACAAATACTTTCCGTCAGACTCTGGAGTGA | | |
| | GAATCATAGCTGACCCCGGCAGATACTATGTTGCATCAGCTTTCACGCTTGCAGTTAA | | |
| | TATCATTGCCAAGAAAATTGTATTAAAGGAACAGACGGGCTCTGATGACGAAGATGAG | | |
| | TCGAGTGAGCAGACCTTTATGTATTATGTGAATGATGGCGTCTATGGATCATTTAATT | | |
| | GCATACTCTATGACCACGCACATGTAAAGCCCCTTCTGCAAAAGAGACCTAAACCAGA | | |
| | TGAGAAGTATTATTCATCCAGCATATGGGGACCAACATGTGATGGCCTCGATCGGATT | | |
| | GTTGAGCGCTGTGACCTGCCTGAAATGCATGTGGGTGATTGGATGCTCTTTGAAAACA | | |
| | TGGGCGCTTACACTGTTGCTGCTGCCTCTACGTTCAATGGCTTCCAGAGGCCGACGAT | | |
| | CTACTATGTGATGTCAGGGCCTGCGTGGCAACTCATGCAGCAATTCCAGAACCCCGAC | | |
| | TTCCCACCCGAAGTAGACGAACAGGATGCCAGCACCCTGCCTGTGTCTTGTGCCTGGG | | |
| | AGAGTGGGATGAAACGCCACAGAGCAGCCTGTGCTTCGGCTAGTATTAATGTGTAGAT | | |
| | AGCACTCTGGTAGCTGTTAACTGCAAGTTTAGCTTGAATTAAGGGATTTGGGGGGACC | | |
| | ATGTAACTTAATTACTGCTAGTTTTGAAATGTCTTTGTAAGAGTAGGGTCGCCATGAT | | |
| | GCAGCCATATGGAAGACTAGGATATGGGTCACACTTATCTGTGTTCCTATGGAAACTA | | |
| | TTTGAATATTTGTTTTATATGGATTTTTATTCACTCTTCAGACACGCTACTCAAGAGT | | |
| | GCCCCTCAGCTGCTGAACAAGCATTTGTAGCTTGTACAATGGCAGAATGGGCCAAAAG | | |
| | CTTAGTGTTGTGACCTGTTTTTAAAATAAAGTATCTTGAAATAACAAAAAAAAAAAAA | | |
| | GGGGGGCCGCCCTAGGGGTTCCCAAGTTTACGTACGCTGCATGG | | |
| | ORF Start: ATG at 179 | | ORF Stop: TAG at 1562 |
| | SEQ ID NO:112 | 461 aa | MW at 51147.6 kD |
| NOV10b, | MNNFGNEEFDCHFLDEGFTAKDILDQKINEVSSSDDKDAFYVADLGDILKKHLRWLKA | | |
| CG124907-01 | | | |
| Protein Sequence | LPRVTPFYAVKCNDSKAIVKTLAATGTGFDCASKTEIQLVQSLGVPPERIIYANPCKQ | | |
| | VSQTKYAANNGVQMMTFDSEVELMKVARAHPKAKLVLRIATDDSKAVCRLSVKFGATL | | |
| | RTSRLLLERAKELNIDVVGVSFHVGSGCTDPETFVQAISDARCVFDMGAEVGFSMYLL | | |
| | DIGGGFPGSEDVKLKFEEITGVINPALDKYFPSDSGVRIIAEPGRYYVASAFTLAVNI | | |
| | IAKKIVLKEQTGSDDEDESSEQTFMYYVNDGVYGSFNCILYDHAHVKPLLQKRPKPDE | | |
| | KYYSSSTWGPTCDGLDRIVERCDLPEMHVGDWMLFEMMGAYTVAAASTFNGFQRPTIY | | |
| | YVMSGPAWQLMQQFQNPDFPPEVEEQDASTLPVSCAWESGMKRHRAACASASINV | | |
| | SEQ ID NO:113 | 1416 bp | |
| NOV10c, | CGCGGATCCACCATGAACAACTTTGGTAATGAAGAGTTTGACTGCCACTTCCTCGATG | | |
| 254048022 DNA | | | |
| Sequence | AAGGTTTTACTCCCAAGGACATTCTGGACCAGAAAATTAATGAAGTTTCTTCTTCTGA | | |
| | TGATAAGGATGCCTTCTATGTGGCAGACCTGGGAGACATTCTAAAGAAACATCTGAGG | | |
| | TGGTTAAAAGCTCTCCCTCGTGTCACCCCCTTTTATGCAGTCAAATGTAATGATAGCA | | |
| | AAGCCATCGTGAAGACCCTTGCTGCTACCGGGACAGGATTTGACTGTGCTAGCAAGAC | | |
| | TGAAATACAGTTGGTGCAGAGTCTGGGGGTGCCTCCAGAGAGGATTATCTATGCAAAT | | |
| | CCTTGTAAACAAGTATCTCAAATTAAGTATGCTGCTAATAATGGAGTCCAGATGATGA | | |
| | CTTTTGATAGTGAAGTTGAGTTGATGAAAGTTGCCAGAGCACATCCCAAAGCAAAGTT | | |
| | GGTTTTGCGGATTGCCACTGATGATTCCAAAGCAGTCTGTCGTCTCAGTGTGAAATTC | | |
| | GGTGCCACGCTCAGAACCAGCAGGCTCCTTTTGGAACGGGCGAAAGAGCTAAATATCG | | |
| | ATGTTGTTGGTGTCAGCTTCCATGTAGGAAGCGGCTGTACCGATCCTGAGACCTTCGT | | |
| | GCAGGCAATCTCTGATGCCCGCTGTGTTTTTGACATGGGGGCTGAGGTTGGTTTCAGC | | |
| | ATGTATCTGCTTGATATTGGCGGTGGCTTTCCTGGATCTGAGGATGTGAAACTTAAAT | | |
| | TTGAAGAGATCACCGGCGTAATCAACCCAGCGTTGGACAAATACTTTCCGTCAGACTC | | |
| | TGGAGTGAGAATCATAGCTGAGCCCGGCAGATACTATGTTGCATCAGCTTTCACGCTT | | |
| | GCAGTTAATATCATTGCCAAGAAAATTGTATTAAAGGAACAGACGGGCTCTGATGACG | | |
| | AAGATGAGTCGAGTGAGCAGACCTTTATGTATTATGTGAATGATGGCGTCTATGGATC | | |
| | ATTTAATTGCATACTCTATGACCACGCACATGTAAAGCCCCTTCTGCAAAAGAGACCT | | |
| | AAACCAGATGAGAAGTATTATTCATCCAGCATATGGGGACCAACATGTGATGGCCTCG | | |
| | ATCGGATTGTTGAGCGCTGTGACCTGCCTGAAATGCATGTGGGTCATTGGATGCTCTT | | |
| | TGAAAACATGGGCGCTTACACTGTTGCTCCTGCCTCTACGTTCAATGGCTTCCAGAGG | | |
| | CCGACGATCTACTATGTGATGTCAGCGCCTGCGTGGCAACTCATGCAGCAATTCCAGA | | |
| | ACCCTGACTTCCCACCCGAAGTAGAGCAACAGGATGCCAGCACCCTGCCTGTGTCTTG | | |
| | TGCCTGGGAGAGTGGGATGAAACGCCACAGAGCAGCCTGTGCTTCGGCTAGTATTAAT | | |
| | GTGTAGGCGGCCGCTTTTTTCCTT | | |
| | ORF Start: at 1 | | ORF Stop: TAG at 1396 |
| | SEQ ID NO:114 | 465 aa | MW at 51549.0 kD |
| NOV10c, | RGSTMNNFGNEEFDCHFLDEGFTAKDILDQKINEVSSSDDKDAFYVADLGDILKKHLR | | |
| 254048022 | | | |
| Protein Sequence | WLKALPRVTPFYAVKCNDSKAIVKTLAATGTGFDCASKTEIQLVQSLGVPPERIIYAN | | |
| | PCKQVSQIKYAANNGVQMMTFDSEVELMKVARAHPKAKLVLRIATDDSKAVCRLSVKF | | |
| | GATLRTSRLLLERAKELNIDVVGVSFHVGSGCTDPETFVQAISDARCVFDMGAEVGFS | | |
| | MYLLDIGGGFPGSEDVKLKFEEITGVINPALDKYFPSDSGVRIIAEPCRYYVASAFTL | | |
| | AVNIIAKKIVLKEQTGSDDEDESSEQTFMYYVNDGVYGSFNCILYDHAHVKPLLQKRP | | |
| | KPDEKYYSSSIWGPTCDGLDRIVERCDLPEMHVGDWMLFENNGAYTVAAASTFNGFQR | | |
| | PTIYYVMSGPAWQLMQQFQNPDFPPEVEEQDASTLPVSCAWESGMKRHRAACASASIN | | |
| V | | | |
| | SEQ ID NO:115 | 1410 bp | |
| NOV10d, | ACCATGGGCCACCATCACCACCATCACAACAACTTTGGTAATGAAGAGTTTGACTGCC | | |
| 258252457 DNA | | | |
| Sequence | ACTTCCTCGATGAAGGTTTTACTGCCAAGGACATTCTGGACCAGAAAATTAATGAAGT | | |
| | TTCTTCTTCTGATGATAAGGATGCCTTCTATGTGGCAGACCTGGGAGACATTCTAAAG | | |
| | AAACATCTGAGGTGGTTAAAAGCTCTCCCTCGTGTCACCCCCTTTTATGCAGTCAAAT | | |
| | GTAATGATAGCAAAGCCATCGTGAAGACCCTTGCTGCTACCGGGACAGGATTTGACTG | | |
| | TGCTAGCAAGACTGAAATACAGTTGGTGCAGAGTCTGGGGGTGCCTCCAGAGAGGATT | | |
| | ATCTATGCAAATCCTTGTAAACAAGTATCTCAAATTAAGTATGCTGCTAATAATGGAG | | |
| | TCCAGATGATGACTTTTGATAGTGAAGTTGAGTTGATGAAAGTTGCCAGAGCACATCC | | |
| | CAAAGCAAAGTTGGTTTTGCGGATTGCCACTGATGATTCCAAAGCAGTCTGTCGTCTC | | |
| | AGTGTGAAATTCGGTGCCACGCTCAGAACCAGCAGGCTCCTTTTGGAACGGGCGAAAG | | |
| | AGCTAAATATCGATGTTGTTGGTGTCAGCTTCCATGTAGGAAGCGGCTGTACCGATCC | | |
| | TGAGACCTTCGTGCAGGCAATCTCTGATGCCCGCTGTGTTTTTGACATGGGGGCTGAG | | |
| | GTTGGTTTCAGCATGTATCTGCTTGATATTGGCGGTGGCTTTCCTGGATCTGAGGATG | | |
| | TGAAACTTAAATTTGAAGAGATCACCGGCGTAATCAACCCAGCGTTGGACAAATACTT | | |
| | TCCGTCAGACTCTGGAGTGAGAATCATAGCTGAGCCCGGCAGATACTATGTTGCATCA | | |
| | GCTTTCACGCTTGCAGTTAATATCATTGCCAAGAAAATTGTATTAAAGGAACAGACGG | | |
| | GCTCTGATGACGAAGATGAGTCGAGTGAGCAGACCTTTATGTATTATGTGAATGATGG | | |
| | CGTCTATGGATCATTTAATTGCATACTCTATGACCACGCACATGTAAAGCCCCTTCTG | | |
| | CAAAAGAGACCTAAACCAGATGAGAAGTATTATTCATCCAGCATATGGGGACCAACAT | | |
| | GTGATGGCCTCGATCGGATTGTTGAGCGCTGTGACCTGCCTGAAATGCATGTGGGTGA | | |
| | TTGGATGCTCTTTGAAAACATGGGCGCTTACACTGTTGCTGCTGCCTCTACGTTCAAT | | |
| | GGCTTCCAGAGGCCGACGATCTACTATGTGATGTCAGGGCCTGCGTGGCAACTCATGC | | |
| | ACCAATTCCAGAACCCTGACTTCCCACCCGAAGTAGAGGAACAGGATGCCACCACCCT | | |
| | GCCTGTGTCTTGTGCCTGGGAGAGTGGGATGAAACGCCACAGAGCAGCCTGTGCTTCG | | |
| | GCTAGTATTAATGTGTAG | | |
| | ORF Start: at 1 | | ORF Stop: TAG at 1408 |
| | SEQ ID NO:116 | 469 aa | MW at 52128.6 kD |
| NOV10d, | TMGHHHHHHNNFGNEEFDCHFLDEGFTAKDILDQKINEVSSSDDKDAFYVADLGDILK | | |
| 258252457 | | | |
| Protein Sequence | KHLRWLKALPRVTPFYAVKCNDSKAIVKTLAATGTGFDCASKTEIQLVQSLGVPPERI | | |
| | IYANPCKQVSQIKYAANNGVQMMTFDSEVELMKVARAHPKAKLVLRIATDDSKAVCRL | | |
| | SVKFGATLRTSRLLLERAKELNIDVVGVSFHVGSGCTDPETFVQAISDARCVFDMGAE | | |
| | VGFSMYLLDIGGGFPGSEDVKLKFEEITGVINPALDKYFPSDSGVRIIAEPGRYYVAS | | |
| | AFTLAVNIIAKKIVLKEQTGSDDEDESSEQTFMYYVNDGVYGSFNCILYDHAHVKPLL | | |
| | QKRPKPDEKYYSSSIWGPTCDGLDRIVERCDLPEMHVGDWMLFENMGAYTVAAASTFN | | |
| | GFQRPTIYYVMSGPAWQLMQQFQNPDFPPEVEEQDASTLPVSCAWESGMKRHRAACAS | | |
| | ASINV | | |
| | SEQ ID NO:117 | 1407 bp | |
| NOV10e, | ACCATGAACAACTTTGGTAATGAAGAGTTTGACTGCCACTTCCTCCATGAAGGTTTTA | | |
| 258280014 DNA | | | |
| Sequence | CTGCCAAGGACATTCTGGACCAGAAAATTAATGAAGTTTCTTCTTCTGATGATAAGGA | | |
| | TGCCTTCTATGTGGCAGACCTGGGAGACATTCTAAAGAAACATCTGACGTGGTTAAAA | | |
| | GCTCTCCCTCGTGTCACCCCCTTTTATGCAGTCAAATGTAATGATAGCAAAGCCATCG | | |
| | TGAAGACCCTTGCTGCTACCGGGACAGGATTTGACTGTGCTAGCAAGACTGAAATACA | | |
| | GTTGGTGCAGAGTCTGGGGGTGCCTCCAGAGAGGATTATCTATGCAAATCCTTGTAAA | | |
| | CAAGTATCTCAAATTAAGTATGCTGCTAATAATGGAGTCCAGATGATGACTTTTGATA | | |
| | GTGAAGTTGAGTTGATGAAAGTTGCCAGAGCACATCCCAAAGCAAAGTTGGTTTTGCG | | |
| | GATTGCCACTGATGATTCCAAAGCAGTCTGTCGTCTCAGTGTGAAATTCGGTGCCACG | | |
| | CTCAGAACCAGCAGGCTCCTTTTGGAACGGGCGAAAGAGCTAAATATCGATGTTGTTG | | |
| | GTGTCAGCTTCCATGTAGGAACCGGCTGTACCGATCCTGAGACCTTCGTGCAGGCAAT | | |
| | CTCTGATGCCCGCTGTGTTTTTGACATGGGGGCTGAGGTTGGTTTCAGCATGTATCTG | | |
| | CTTGATATTGGCGGTGGCTTTCCTGGATCTGAGGATGTGAAACTTAAATTTGAAGAGA | | |
| | TCACCGGCGTAATCAACCCAGCGTTGGACAAATACTTTCCGTCAGACTCTGGAGTGAG | | |
| | AATCATAGCTGAGCCCGGCAGATACTATGTTGCATCAGCTTTCACGCTTGCAGTTAAT | | |
| | ATCATTGCCAAGAAAATTGTATTAAAGGAACAGACGGGCTCTGATGACCAAGATGAGT | | |
| | CGAGTGAGCAGACCTTTATGTATTATGTGAATGATGGCGTCTATGGATCATTTAATTG | | |
| | CATACTCTATGACCACGCACATGTAAAGCCCCTTCTGCAAAAGAGACCTAAACCAGAT | | |
| | GAGAAGTATTATTCATCCAGCATATGGGGACCAACATGTGATGGCCTCGATCGGATTG | | |
| | TTGAGCGCTGTGACCTGCCTGAAATGCATGTGGGTGATTGGATGCTCTTTGAAAACAT | | |
| | GGGCGCTTACACTGTTGCTGCTGCCTCTACGTTCAATGGCTTCCAGAGGCCGACGATC | | |
| | TACTATGTGATGTCAGGGCCTGCGTGGCAACTCATGCAGCAATTCCAGAACCCTGACT | | |
| | TCCCACCCGAAGTAGAGGAACAGGATGCCAGCACCCTGCCTGTGTCTTGTGCCTGGGA | | |
| | GAGTGGGATGAAACGCCACAGAGCAGCCTGTGCTTCGGCTAGTATTAATGTGCACCAT | | |
| | CACCACCATCACTGA | | |
| | ORF Start: at 1 | | ORF Stop: TGA at 1405 |
| | SEQ ID NO:118 | 468 aa | MW at 5207l.6 kD |
| NOV10e, | TMNNFGNEEFDCHFLDEGFTAKDILDQKINEVSSSDDKDAFYVADLGDILKKHLRWLK | | |
| 258280014 | | | |
| Protein Sequence | ALPRVTPFYAVKCNDSKAIVKTLAATGTGFDCASKTEIQLVQSLGVPPERIIYANPCK | | |
| | QVSQIKYAANNGVQMMTFDSEVELMKVARAHPKAKLVLRIATDDSKAVCRLSVKFGAT | | |
| | LRTSRLLLERAKELNIDVVGVSFHVGSGCTDPETFVQAISDARCVFDMGAEVGFSMYL | | |
| | LDIGGGFPGSEDVKLKFEEITGVINPALDKYFPSDSGVRIIAEPGRYYVASAFTLAVN | | |
| | IIAKKIVLKEQTGSDDEDESSEQTFMYYVNDGVYGSFNCILYDHAHVKPLLQKRPKPD | | |
| | EKYYSSSIWGPTCDGLDRIVERCDLPEMHVGDWMLFENMGAYTVAAASTFNGFQRPTI | | |
| | YYVMSGPAWQLMQQFQNPDFPPEVEEQDASTLPVSCAWESGMKRHRAACASASINVHH | | |
| | HHHH | | |
| | SEQ ID NO:119 | 1434 bp | |
| NOV10f, | CACCATCACCACCATCACAACAACTTTGGTAATGAAGAGTTTGACTGCCACTTCCTCG | | |
| 258330318 DNA | | | |
| Sequence | ATGAAGGTTTTACTGCCAAGGACATTCTGGACCAGAAAATTAATGAAGTTTCTTCTTC | | |
| | TGATGATAAGGATGCCTTCTATGTGGCAGACCTGGGAGACATTCTAAAGAAACATCTG | | |
| | AGGTGGTTAAAAGCTCTCCCTCGTGTCACCCCCTTTTATGCAGTCAAATGTAATGATA | | |
| | GCAAAGCCATCGTCAAGACCCTTGCTGCTACCGGGACAGGATTTGACTGTGCTAGCAA | | |
| | GACTGAAATACAGTTGGTGCAGAGTCTGGGGGTGCCTCCAGAGAGGATTATCTATGCA | | |
| | AATCCTTCTAAACAAGTATCTCAAATTAAGTATGCTGCTAATAATGGAGTCCAGATGA | | |
| | TGACTTTTGATAGTGAAGTTGAGTTGATGAAAGTTGCCAGAGCACATCCCAAAGCAAA | | |
| | GTTGGTTTTGCGGATTGCCACTGATGATTCCAAAGCAGTCTGTCGTCTCAGTGTGAAA | | |
| | TTCGGTGCCACGCTCAGAACCAGCAGGCTCCTTTTGGAACGGGCGPAAGAGCTAAATA | | |
| | TCGATGTTGTTGGTGTCAGCTTCCATGTAGGAAGCGGCTGTACCGATCCTGAGACCTT | | |
| | CGTGCAGGCAATCTCTGATGCCCGCTGTGTTTTTGACATGGCGGCTGAGGTTGGTTTC | | |
| | AGCATGTATCTGCTTGATATTGGCGGTGGCTTTCCTGGATCTGAGGATGTGAAACTTA | | |
| | AATTTGAAGAGATCACCGGCGTAATCAACCCAGCGTTGGACAAATACTTTCCGTCAGA | | |
| | CTCTGGAGTGAGAATCATAGCTGAGCCCGGCAGATACTATGTTGCATCAGCTTTCACG | | |
| | CTTGCAGTTAATATCATTGCCAAGAAAATTGTATTAAAGGAACAGACGGGCTCTGATG | | |
| | ACGAAGATGAGTCGAGTGAGCAGACCTTTATGTATTATGTGAATGATGGCGTCTATGG | | |
| | ATCATTTAATTGCATACTCTATGACCACGCACATGTAAAGCCCCTTCTGCAAAAGAGA | | |
| | CCTAAACCAGATGAGAAGTATTATTCATCCAGCATATGGGGACCAACATGTGATGGCC | | |
| | TCGATCGGATTGTTGAGCGCTGTGACCTGCCTGAAATGCATGTGGGTGATTGGATGCT | | |
| | CTTTGAAAACATGGGCGCTTACACTGTTGCTGCTGCCTCTACGTTCAATGGCTTCCAG | | |
| | AGGCCGACGATCTACTATGTGATGTCAGGGCCTGCGTGGCAACTCATGCAGCAATTCC | | |
| | AGAACCCTGACTTCCCACCCGAAGTAGAGGAACAGGATGCCAGCACCCTGCCTGTGTC | | |
| | TTGTGCCTGGGAGAGTGGGATGAAACGCCACAGAGCAGCCTGTGCTTCGGCTAGTATT | | |
| | AATGTGTAGGCGCCCGCACTCGAGCACCACCACCACCACCAC | | |
| | ORF Start: at 1 | | ORF Stop: TAG at 1399 |
| | SEQ ID NO:120 | 466 aa | MW at 51839.3 kD |
| NOV10f | HHHHNHNNFGNEEFDCHFLDEGFTAKDILDQKINEVSSSDDKDAFYVADLGDILKKHL | | |
| 258330318 | | | |
| Protein Sequence | RWLKALPRVTPFYAVKCNDSKAIVKTLAATGTGFDCASKTEIQLVQSLGVPPERIIYA | | |
| | NPCKQVSQTKYAANNGVQMMTFDSEVELMKVARAHPKAKLVLRIATDDSKAVCRLSVK | | |
| | FGATLRTSRLLLERAKELNIDVVGVSFHVGSGCTDPETFVQAISDARCVFDMGAEVGF | | |
| | SMYLLDIGGGFPGSEDVKLKFEEITGVINPALDKYFPSDSGVRIIAEPGRYYVASAFT | | |
| | LAVNIIAKKIVLKEQTGSDDEDESSEQTFMYYVNDGVYGSFNCILYDHAHVKPLLQKR | | |
| | PKPDEKYYSSSIWGPTCDGLDRIVERCDLPEMHVGDWMLFENMGAYTVAAASTFNGFQ | | |
| | RPTIYYVMSGPAWQLMQQFQNPDFPPEVEEQDASTLPVSCAWESGMKRHRAACASASI | | |
| | NV | | |
| | SEQ ID NO:121 | 1305 bp | |
| NOV10g, | ACATCATCACCACCATCAAACAACTTTGGTAATGAAGAGTTTCACTGCCACTTCCTCG | | |
| 258330346 DNA | | | |
| Sequence | ATGAAGGTTTTACTGCCAACGACATTCTGGACCAGAAAATTAATGAAGTTTCTTCTTC | | |
| | TGATGATAAGGATGCCTTCTATGTGGCAGACCTGGGAGACATTCTAAAGAAACATCTG | | |
| | AGGTGGTTAAAAGCTCTCCCTCGTGTCACCCCCTTTTATGCAGTCAAATGTAATGATA | | |
| | GCAAAGCCATCGTGAAGACCCTTGCTGCTACCGGGACAGGATTTGACTGTGCTAGCAA | | |
| | GACTGAAATACAGTTGGTGCAGAGTCTGGGGGTGCCTCCAGAGAGGATTATCTATGCA | | |
| | AATCCTTGTAAACAAGTATCTCAAATTAAGTATGCTGCTAATAATGGAGTCCAGATGA | | |
| | TGACTTTTGATAGTGAAGTTGAGTTGATGAAAGTTGCCAGAGCACATCCCAAAGCAAA | | |
| | GTTGGTTTTGCGGATTGCCACTGATGATTCCAAAGCAGTCTGTCGTCTCAGTGTGAAA | | |
| | TTCGGTGCCACGCTCAGAACCAGCAGGCTCCTTTTGGAACGGGCGAAAGAGCTAAATA | | |
| | TCGATGTTGTTGGTGTCAGCTTCCATGTAGGAAGCGGCTGTACCGATCCTGAGACCTT | | |
| | CGTGCAGGCAATCTCTGATGCCCGCTGTGTTTTTGACATGGGGGCTGAGGTTGGTTTC | | |
| | AGCATGTATCTGCTTGATATTGGCGGTGGCTTTCCTGGATCTGAGGATGTGAAACTTA | | |
| | AATTTGAAGAGATCACCGGCGTAATCAACCCAGCGTTGGACAAATACTTTCCGTCAGA | | |
| | CTCTGGAGTGAGAATCATAGCTGAGCCCGGCAGATACTATGTTGCATCAGCTTTCACG | | |
| | CTTGCAGTTAATATCATTGCCAAGAAAATTGTATTAAAGGAACAGACGGGCTCTGATG | | |
| | ACGAAGATGAGTCGAGTGAGCAGACCTTTATGTATTATGTGAATGATGGCGTCTATGG | | |
| | ATCATTTAATTGCATACTCTATGACCACGCACATGTAAAGCCCCTTCTGCAAAAGAGA | | |
| | CCTAAACCAGATGAGAAGTATTATTCATCCAGCATATGGGGACCAACATGTGATGGCC | | |
| | TCGATCGGATTGTTGAGCGCTGTGACCTGCCTGAAATGCATGTGGGTGATTGGATGCT | | |
| | CTTTGAAAACATGGGCGCTTACACTGTTGCTGCTGCCTCTACGTTCAATGGCTTCCAG | | |
| | AGGCCGACGATCTACTATGTGATGTCAGGGCCTGCGTGGCAACTCATGCAGTAGGCGG | | |
| | CCGCACTCGAGCACCACCACCACCACCAC | | |
| | ORF Start: at 1 | | ORF Stop: TAG at 1270 |
| | SEQ ID NO:122 | 423 aa | MW at 46885.9 kD |
| NOV10g, | TSSPPSNNFGNEEFDCHFLDEGFTAKDILDQKIMEVSSSDDKDAFYVADLGDILKKHL | | |
| 258330346 | | | |
| Protein Sequence | RWLKALPRVTPFYAVKCNDSKAIVKThAATGTGFDCASKTEIQLVQSLGVPPERIIYA | | |
| | NPCKQVSQIKYAANNGVQMMTFDSEVELMKVARAHPKAKLVLRIATDDSKAVCRLSVK | | |
| | FGATLRTSRLLLERAKELNIDVVGVSFHVGSGCTDPETFVQAISDARCVFDMGAEVGF | | |
| | SMYLLDIGGGFPGSEDVKLKFEEITGVINPALDKYFPSDSGVRIIAEPGRYYVASAFT | | |
| | LAVNIIAKKIVLKEQTGSDDEDESSEQTFMYYVNDGVYGSFNCILYDHAHVKPLLQKR | | |
| | PKPDEKYYSSSIWGPTCDGLDRIVERCDLPEMHVGDWMLFENMGAYTVAAASTFNGFQ | | |
| | RPTIYYVMSGPAWQLMQ | | |
| | SEQ ID NO:123 | 1389 bp | |
| NOV10h, | ACCATGAACAACTTTGGTAATGAAGAGTTTGACTGCCACTTCCTCGATGAAGGTTTTA | | |
| 258330472 DNA | | | |
| Sequence | CTGCCAAGGACATTCTGGACCAGAAAATTAATGAAGTTTCTTCTTCTGATGATAAGGA | | |
| | TGCCTTCTATGTGGCAGACCTGGGAGACATTCTAAAGAAACATCTGAGGTGGTTAAAA | | |
| | GCTCTCCCTCGTGTCACCCCCTTTTATGCAGTCAAATGTAATGATAGCAAAGCCATCG | | |
| | TGAAGACCCTTGCTGCTACCGGGACAGGATTTGACTGTGCTAGCAAGACTGAAATACA | | |
| | GTTGGTGCAGAGTCTGGGGGTGCCTCCAGAGAGGATTATCTATGCAAATCCTTGTAAA | | |
| | CAAGTATCTCAAATTAAGTATGCTGCTAATAATGGAGTCCAGATGATGACTTTTGATA | | |
| | GTGAAGTTGAGTTGATGAAAGTTGCCAGAGCACATCCCAAAGCAAAGTTGGTTTTGCG | | |
| | GATTGCCACTGATGATTCCAAAGCAGTCTGTCGTCTCAGTGTGAAATTCGGTGCCACG | | |
| | CTCAGAACCAGCAGGCTCCTTTTGCAACGGGCGAAAGAGCTAAATATCGATGTTGTTG | | |
| | GTGTCAGCTTCCATGTAGGAAGCGGCTGTACCGATCCTGAGACCTTCGTGCAGGCAAT | | |
| | CTCTGATGCCCGCTGTGTTTTTGACATGGGGGCTGAGGTTGGTTTCAGCATGTATCTG | | |
| | CTTGATATTGGCGGTGGCTTTCCTGGATCTGAGGATGTGAAACTTAAATTTGAAGAGA | | |
| | TCACCGGCGTAATCAACCCAGCGTTGGACAAATACTTTCCGTCAGACTCTGGAGTGAG | | |
| | AATCATAGCTGAGCCCGGCAGATACTATGTTGCATCAGCTTTCACGCTTGCAGTTAAT | | |
| | ATCATTGCCAAGAAAATTGTATTAAAGGAACAGACGGGCTCTCATGACGAAGATGAGT | | |
| | CGAGTGAGCAGACCTTTATGTATTATGTGAATGATGGCGTCTATGGATCATTTAATTG | | |
| | CATACTCTATGACCACGCACATGTAAAGCCCCTTCTGCAAAAGAGACCTAAACCAGAT | | |
| | GAGAAGTATTATTCATCCAGCATATGGGGACCAACATGTGATGGCCTCGATCGGATTG | | |
| | TTGAGCGCTGTGACCTGCCTGAAATGCATGTGGGTGATTGGATCCTCTTTGAAAACAT | | |
| | GGGCGCTTACACTGTTGCTGCTGCCTCTACGTTCAATGGCTTCCAGAGGCCGACGATC | | |
| | TACTATGTGATGTCAGGGCCTGCGTGGCAACTCATGCAGCAATTCCAGAACCCTGACT | | |
| | TCCCACCCGAAGTAGAGGAACAGGATGCCAGCACCCTGCCTGTGTCTTGTGCCTGGGA | | |
| | GAGTGGGATGAAACGCCACAGAGCAGCCTGTGCTTCGGCTAGTATTAATGTGTAG | | |
| | ORF Start: at 1 | | ORF Stop: TAG at 1387 |
| | SEQ ID NO:124 | 462 aa | MW at 51248.7 kD |
| NOV10h, | TMNNFGNEEFDCHFLDEGFTAKDILDQKINEVSSSDDKDAFYVADLGDILKKHLRWLK | | |
| 258330472 | | | |
| Protein Sequence | ALPRVTPFYAVKCNDSKAIVKTLAATGTGFDCASKTEIQLVQSLGVPPERIIYANPCK | | |
| | QVSQIKYAANNGVQMMTFDSEVELMKVARAHPKAKLVLRIATDDSKAVCRLSVKFGAT | | |
| | LRTSRLLLERAKELNIDVVGVSFHVGSGCTDPETFVQAISDARCVFDMGAEVGFSMYL | | |
| | LDIGCGFPGSEDVKLKFEEITGVINPALDKYFPSDSGVRIIAEPGRYYVASAFTLAVN | | |
| | IIAKKIVLKEQTGSDDEDESSEQTFMYYVNDGVYGSFNCILYDHAHVKPLLQKRPKPD | | |
| | KYYSSSIWGPTCDGLDRIVERCDLPEMHVGDWMLFENMGAYTVAAASTFNGFQRPTIY | | |
| | YYVMSGPAWQLMQQFQNPDFPPEVEEQDASTLPVSCAWESGMKRHRAACASASINV | | |
| | SEQ ID NO:125 | 1386 bp | |
| NOV10i | CATGAACAACTTTGGTAATGAAGAGTTTGACTGCCACTTCCTCGATGAAGGTTTTACT | | |
| 258330611 DNA | | | |
| Sequence | GCCAAGGACATTCTGGACCAGAAAATTAATGAAGTTTCTTCTTCTGATGATAAGGATG | | |
| | CCTTCTATGTGGCAGACCTGGGAGACATTCTAAAGAAACATCTGAGGTGGTTAAAAGC | | |
| | TCTCCCTCGTGTCACCCCCTTTTATGCAGTCAAATGTAATGATAGCAAAGCCATCGTG | | |
| | AAGACCCTTGCTGCTACCGGGACAGGATTTGACTGTGCTAGCAAGACTGAAATACAGT | | |
| | TGGTGCAGAGTCTCGGGGTGCCTCCAGAGAGGATTATCTATGCAAATCCTTGTAAACA | | |
| | AGTATCTCAAATTAAGTATGCTGCTAATAATGGAGTCCAGATGATGACTTTTGATAGT | | |
| | GAAGTTGAGTTGATGAAAGTTGCCAGAGCACATCCCAAAGCAAAGTTGGTTTTGCGGA | | |
| | TTGCCACTGATGATTCCAAAGCAGTCTGTCGTCTCAGTGTGAAATTCGGTGCCACGCT | | |
| | CAGAACCAGCAGGCTCCTTTTGGAACGGGCGAAAGAGCTAAATATCGATGTTGTTGGT | | |
| | GTCAGCTTCCATGTAGGAAGCGGCTGTACCCATCCTGAGACCTTCGTGCAGGCAATCT | | |
| | CTGATGCCCGCTGTGTTTTTGACATGGGGGCTGAGGTTGGTTTCAGCATGTATCTGCT | | |
| | TGATATTGGCGGTGGCTTTCCTGGATCTGAGGATGTGAAACTTAAATTTGAAGAGATC | | |
| | ACCGGCGTAATCAACCCAGCGTTGGACAAATACTTTCCGTCAGACTCTGGAGTGAGAA | | |
| | TCATAGCTGAGCCCGGCAGATACTATGTTCCATCAGCTTTCACGCTTCCAGTTAATAT | | |
| | CATTCCCAAGAAAATTGTATTAAAGGAACAGACGGGCTCTGATGACGAAGATGAGTCG | | |
| | AGTGAGCAGACCTTTATGTATTATGTGAATGATGGCGTCTATGGATCATTTAATTGCA | | |
| | TACTCTATGACCACGCACATGTAAAGCCCCTTCTGCAAAAGAGACCTAAACCAGATGA | | |
| | GAAGTATTATTCATCCAGCATATGGGGACCAACATGTGATGGCCTCGATCGGATTGTT | | |
| | GAGCGCTGTGACCTGCCTGAAATGCATGTGGGTGATTGGATGCTCTTTGAAAACATGG | | |
| | GCGCTTACACTGTTGCTGCTGCCTCTACGTTCAATGGCTTCCAGAGGCCGACGATCTA | | |
| | CTATGTGATGTCAGGGCCTGCGTGGCAACTCATGCAGCAATTCCAGAACCCTGACTTC | | |
| | CCACCCGAAGTAGAGGAACAGGATGCCAGCACCCTGCCTGTGTCTTGTGCCTGGGAGA | | |
| | GTGGGATGAAACGCCACAGAGCAGCCTGTGCTTCGGCTAGTATTAATGTGTA | | |
| | ORF Start: ATG at 2 | | ORF Stop: end of |
| | | | sequence |
| | SEQ ID NO:126 | 1462 aa | MW at 51147.6 kD |
| NOV10I, | MNNFGNEEFDCHFLDEGFTAKDILDQKINEVSSSDDKDAFYVADLGDILKKHLRWLKA | | |
| 258330611 | | | |
| Protein Sequence | LPRVTPFYAVKCNDSKAIVKTLAATGTGFDCASKTEIQLVQSLGVPPERIIYANPCKQ | | |
| | VSQIKYAANNGVQMMTFDSEVELMKVARAHPKAKLVLRIATDDSKAVCRLSVKFGATL | | |
| | RTSRLLLERAKELNIDVVGVSFHVGSGCTDPETFVQAISDARCVFDMGAEVGFSMYLL | | |
| | DIGGGFPGSEDVKLKFEEITGVINPALDKYFPSDSGVRIIAEPGRYYVASAFTLAVNI | | |
| | IAKKIVLKEQTGSDDEDESSEQTFMYYVNDGVYGSFNCILYDHAHVKPLLQKRPKPDE | | |
| | KYYSSSIWGPTCDGLDRIVERCDLPEMHVGDWMLFENMGAYTVAAASTFNGFQRPTIY | | |
| | YVMSGPAWQLMQQFQNPDFPPEVEEQDASTLPVSCAWESGMKRHPAACASASINVX | | |
| | SEQ ID NO:127 | 1305 bp | |
| NOV10j, | CACCATCACCACCATCACAACAACTTTGGTAATGAAGAGTTTGACTGCCACTTCCTCG | | |
| 260481330 DNA | | | |
| Sequence | ATGAAGGTTTTACTGCCAAGGACATTCTGGACCAGAAAATTAATGAAGTTTCTTCTTC | | |
| | TGATGATAAGGATGCCTTCTATGTGGCAGACCTGGGAGACATTCTAAAGAAACATCTG | | |
| | AGGTGGTTAAAAGCTCTCCCTCGTGTCACCCCCTTTTATGCAGTCAAATGTAATGATA | | |
| | GCAAAGCCATCGTGAAGACCCTTCCTGCTACCGGGACAGGATTTGACTGTGCTAGCAA | | |
| | GACTGAAATACAGTTGGTGCAGAGTCTGGGGGTGCCTCCAGAGAGGATTATCTATGCA | | |
| | AATCCTTGTAAACAAGTATCTCAAATTAAGTATGCTGCTAATAATGGAGTCCAGATGA | | |
| | TGACTTTTGATAGTGAAGTTGAGTTGATGAAAGTTGCCAGAGCACATCCCAAAGCAAA | | |
| | GTTGGTTTTGCGGATTGCCACTGATGATTCCAAAGCAGTCTGTCGTCTCAGTGTGAAA | | |
| | TTCGGTGCCACGCTCAGAACCAGCAGGCTCCTTTTGGAACGGGCGAAAGAGCTAAATA | | |
| | TCGATGTTGTTGGTGTCAGCTTCCATGTAGCAAGCGGCTGTACCGATCCTGAGACCTT | | |
| | CGTGCAGGCAATCTCTGATGCCCGCTGTGTTTTTGACATGGGGGCTGAGGTTGGTTTC | | |
| | AGCATGTATCTGCTTGATATTGGCGGTGGCTTTCCTGGATCTGAGGATGTGAAACTTA | | |
| | AATTTGAAGAGATCACCGGCGTAATCAACCCAGCGTTGGACAAATACTTTCCGTCAGA | | |
| | CTCTGGAGTGAGAATCATAGCTGAGCCCGGCAGATACTATGTTGCATCAGCTTTCACG | | |
| | CTTGCAGTTAATATCATTGCCAAGAAAATTGTATTAAAGGAACAGACGGGCTCTGATG | | |
| | ACGAAGATGAGTCGAGTGAGCAGACCTTTATGTATTATGTGAATGATGGCGTCTATGG | | |
| | ATCATTTAATTGCATACTCTATGACCACGCACATGTAAAGCCCCTTCTGCAAAAGAGA | | |
| | CCTAAACCAGATGAGAAGTATTATTCATCCAGCATATGGGGACCAACATGTGATGGCC | | |
| | TCGATCGGATTGTTGAGCGCTGTGACCTGCCTGAAATGCATGTGGGTGATTGGATGCT | | |
| | CTTTGAAAACATGGGCGCTTACACTGTTGCTGCTGCCTCTACGTTCAATGGCTTCCAG | | |
| | AGGCCGACGATCTACTATGTGATGTCAGGGCCTCCGTGGCAACTCATGCAGTAGGCGG | | |
| | CCGCACTCGAGCACCACCACCACCACCAC | | |
| | ORF Start: at 1 | | ORF Stop: TAG at 1270 |
| | SEQ ID NO:128 | 423 aa | MW at 47152.2 kD |
| NOV10j, | HHHHHHNNFGNEEFDCHFLDEGFTAKDILDQKINEVSSSDDKDAFYVADLGDILKKHL | | |
| 260481330 | | | |
| Protein Sequence | RWLKALPRVTPFYAVKCNDSKAIVKTLAATGTGFDCASKTEIQLVQSLGVPPERIIYA | | |
| | NPCKQVSQIKYAANNGVQMMTFDSEVELMKVARAHPKAKLVLRIATDDSKAVCRLSVK | | |
| | FGATLRTSRLLLERAKELNIDVVGVSFHVGSGCTDPETFVQAISDARCVFDMGAEVGF | | |
| | SMYLLDIGGGFPGSEDVKLKFEEITGVINPALDKYFPSDSGVRIIAEPGRYYVASAFT | | |
| | LAVNIIAKKIVLKEQTGSDDEDESSEQTFMYYVNDGVYGSFNCILYDHAHVKPLLQKR | | |
| | PKPDEKYYSSSIWGPTCDGLDRIVERCDLPEMHVGDWMLFENMGAYTVAAASTFNGFQ | | |
| | RPTIYYVMSGPAWQLMQ | | |
| | SEQ ID NO:129 | 1416 bp | |
| NOV10k, | CGCGGATCCACCATGAACAACTTTGGTAATGAAGAGTTTGACTGCCACTTCCTCGATG | | |
| CG124907-02 | | | |
| DNA Sequence | AAGGTTTTACTGCCAAGGACATTCTGGACCAGAAAATTAATGAAGTTTCTTCTTCTGA | | |
| | TGATAAGGATGCCTTCTATGTGGCAGACCTGGGAGACATTCTAAACAAACATCTGAGG | | |
| | TGGTTAAAAGCTCTCCCTCGTGTCACCCCCTTTTATGCAGTCAAATGTAATGATAGCA | | |
| | AAGCCATCGTGAAGACCCTTGCTGCTACCGGGACAGGATTTGACTGTGCTAGCAAGAC | | |
| | TGAAATACAGTTGGTGCAGAGTCTGGGGGTGCCTCCAGAGAGGATTATCTATGCAAAT | | |
| | CCTTGTAAACAAGTATCTCAAATTAAGTATGCTGCTAATAATGGAGTCCAGATGATGA | | |
| | CTTTTGATAGTGAAGTTGAGTTGATGAAAGTTGCCAGAGCACATCCCAAAGCAAAGTT | | |
| | GGTTTTGCGGATTGCCACTGATGATTCCAAAGCAGTCTGTCGTCTCAGTGTGAAATTC | | |
| | GGTGCCACGCTCAGAACCAGCAGGCTCCTTTTGGAACGGGCGAAAGAGCTAAATATCG | | |
| | ATGTTGTTGGTGTCAGCTTCCATGTAGGAAGCGGCTGTACCGATCCTGAGACCTTCGT | | |
| | GCAGGCAATCTCTGATGCCCGCTGTGTTTTTGACATGGGGGCTGAGGTTGGTTTCAGC | | |
| | ATGTATCTGCTTGATATTGGCGGTGGCTTTCCTGGATCTGAGGATGTGAAACTTAAAT | | |
| | TTGAAGAGATCACCGGCGTAATCAACCCACCGTTGGACAAATACTTTCCGTCAGACTC | | |
| | TGGAGTGAGAATCATAGCTGAGCCCGGCAGATACTATGTTGCATCAGCTTTCACGCTT | | |
| | GCAGTTAATATCATTGCCAAGAAAATTGTATTAAAGGAACAGACGGGCTCTGATGACG | | |
| | AAGATGAGTCGAGTGAGCAGACCTTTATGTATTATGTGAATGATGGCGTCTATGGATC | | |
| | ATTTAATTGCATACTCTATGACCACGCACATGTAAAGCCCCTTCTGCAAAAGACACCT | | |
| | AAACCAGATGAGAAGTATTATTCATCCAGCATATGGGGACCAACATGTGATGGCCTCG | | |
| | ATCGGATTGTTGAGCGCTGTGACCTGCCTGAAATGCATGTGGGTGATTGGATGCTCTT | | |
| | TGAAAACATGGGCGCTTACACTGTTGCTGCTGCCTCTACGTTCAATGGCTTCCAGAGG | | |
| | CCGACGATCTACTATGTGATGTCAGGGCCTGCGTGGCAACTCATGCAGCAATTCCAGA | | |
| | ACCCTGACTTCCCACCCGAAGTAGAGGAACAGGATGCCAGCACCCTGCCTGTGTCTTG | | |
| | TGCCTGGGAGAGTGGGATGAAACGCCACAGAGCAGCCTGTGCTTCGGCTAGTATTAAT | | |
| | GTGTAGGCGGCCGCTTTTTTCCTT | | |
| | ORF Start: ATG at 13 | | ORF Stop: TAG at 1396 |
| | SEQ ID NO:130 | 461 aa | MW at 51147.6 kD |
| NOV10k | MNNFGNEEFDCHFLDEGFTAKDILDQKINEVSSSDDKDAFYVADLGDILKKHLRWLKA | | |
| CG124907-02 | | | |
| Protein Sequence | LPRVTPFYAVKCNDSKAIVKTLAATCTGFDCASKTEIQLVQSLGVPPERIIYANPCKQ | | |
| | VSQIKYAANNGVQMMTFDSEVELMKVAPAHPKAKLVLRIATDDSKAVCRLSVKFGATL | | |
| | RTSRLLLERAKELNIDVVGVSFHVGSGCTDPETFVQAISDARCVFDMGAEVGFSMYLL | | |
| | DIGGGFPGSEDVKLKFEEITGVINPALDKYFPSDSGVRIIAEPGRYYVASAFTLAVNI | | |
| | IAKKIVLKEQTGSDDEDESSEQTFMYYVNDGVYGSFNCILYDHAHVKPLLQKRPKPDE | | |
| | KYYSSSIWGPTCDGLDRIVERCDLPEMHVGDWNLFENMGAYTVAAASTFNGFQRPTIY | | |
| | YVMSGPAWQLMQQFQNPDFPPEVEEQDASTLPVSCAWESGMKRHRAACASASINV | | |
| | SEQ ID NO:131 | 1410 bp | |
| NOV10l, | ACCATGGGCCACCATCACCACCATCACAACAACTTTGGTAATGAAGAGTTTGACTGCC | | |
| CG124907-03 | | | |
| DNA Sequence | ACTTCCTCGATGAAGGTTTTACTGCCAAGGACATTCTGGACCAGAAAATTAATGAAGT | | |
| | TTCTTCTTCTGATGATAAGGATGCCTTCTATGTGGCAGACCTGGGAGACATTCTAAAG | | |
| | AAACATCTGAGGTGGTTAAAAGCTCTCCCTCGTGTCACCCCCTTTTATGCAGTCAAAT | | |
| | GTAATGATAGCAAAGCCATCGTGAAGACCCTTGCTGCTACCGGGACAGGATTTGACTG | | |
| | TGCTAGCAAGACTGAAATACAGTTGGTGCAGAGTCTGGGGGTGCCTCCAGAGAGGATT | | |
| | ATCTATGCAAATCCTTGTAAACAAGTATCTCAAATTAAGTATGCTGCTAATAATGGAG | | |
| | TCCAGATGATGACTTTTGATAGTGAAGTTGAGTTGATGAAAGTTGCCAGAGCACATCC | | |
| | CAAAGCAAAGTTGGTTTTGCGGATTGCCACTGATGATTCCAAAGCAGTCTGTCGTCTC | | |
| | AGTGTGAAATTCGGTGCCACGCTCAGAACCAGCAGGCTCCTTTTGGAACGGGCGAAAG | | |
| | AGCTAAATATCGATGTTGTTGGTGTCAGCTTCCATGTAGGAAGCGGCTGTACCGATCC | | |
| | TGAGACCTTCGTGCAGGCAATCTCTGATGCCCGCTGTGTTTTTGACATGGGGGCTGAG | | |
| | GTTGGTTTCAGCATGTATCTGCTTGATATTGGCGGTGGCTTTCCTGGATCTGAGGATG | | |
| | TGAAACTTAAATTTGAAGAGATCACCGGCGTAATCAACCCAGCGTTGGACAAATACTT | | |
| | TCCGTCAGACTCTGGAGTGAGAATCATAGCTGAGCCCGGCAGATACTATGTTGCATCA | | |
| | GCTTTCACGCTTGCAGTTAATATCATTGCCAAGAAAATTGTATTAAAGGAACAGACGG | | |
| | GCTCTGATGACGAAGATGAGTCGAGTGAGCAGACCTTTATGTATTATGTGAATGATCG | | |
| | CGTCTATGGATCATTTAATTGCATACTCTATGACCACGCACATGTAAAGCCCCTTCTG | | |
| | CAAAAGAGACCTAAACCAGATGAGAAGTATTATTCATCCAGCATATGGGGACCAACAT | | |
| | GTGATGGCCTCGATCGGATTGTTGAGCGCTGTGACCTGCCTGAAATGCATGTGGGTGA | | |
| | TTGGATGCTCTTTGAAAACATGGGCGCTTACACTGTTGCTGCTGCCTCTACGTTCAAT | | |
| | GGCTTCCAGAGGCCGACGATCTACTATGTGATGTCAGCGCCTGCGTGCCAACTCATGC | | |
| | AGCAATTCCAGAACCCTGACTTCCCACCCGAAGTAGAGGAACAGGATGCCAGCACCCT | | |
| | GCCTGTGTCTTGTGCCTGGGAGAGTGGGATGAAACGCCACAGAGCAGCCTGTGCTTCG | | |
| | GCTAGTATTAATGTGTAG | | |
| | ORF Start: at 1 | | ORF Stop: TAG at 1408 |
| | SEQ ID NO:132 | 469 aa | MW at 52128.6 kD |
| NOV10l, | TMGHHHHHHNNFGNEEFDCHFLDEGFTAKDILDQKINEVSSSDDKDAFYVADLGDILK | | |
| CG124907-03 | | | |
| Protein Sequence | KHLRWLKALPRVTPFYAVKCNDSKAIVKTLAATGTGFDCASKTEIQLVQSLGVPPERI | | |
| | IYANPCKQVSQIKYAANNGVQMMTFDSEVELMKVARAHPKAKLVLRIATDDSKAVCRL | | |
| | SVKFGATLRTSRLLLERAKELNIDVVGVSFHVGSGCTDPETFVQAISDARCVFDMGAE | | |
| | VGFSMYLLDIGGGFPGSEDVKLKFEEITGVINPALDKYFPSDSGVRIIAEPGRYYVAS | | |
| | AFTLAVNIIAKKIVLKEQTGSDDEDESSEQTFMYYVNDGVYGSFNCILYDHAHVKPLL | | |
| | QKRPKPDEKYYSSSIWGPTCDGLDRIVERCDLPEMHVGDWMLFENMGAYTVAAASTFN | | |
| | GFQRPTIYYVMSGPAWQLMQQFQNPDFPPEVEEQDASTLPVSCAWESGMKRHRAACAS | | |
| | ASINV | | |
| | SEQ ID NO:133 | 1407 bp | |
| NOV10m, | ACCATGAACAACTTTGGTAATGAAGAGTTTGACTGCCACTTCCTCGATGAAGGTTTTA | | |
| CG124907-04 | | | |
| DNA Sequence | CTGCCAAGGACATTCTGGACCAGAAAATTAATGAAGTTTCTTCTTCTGATGATAAGGA | | |
| | TGCCTTCTATGTGGCAGACCTGGGAGACATTCTAAAGAAACATCTGAGGTGGTTAAAA | | |
| | GCTCTCCCTCGTGTCACCCCCTTTTATGCAGTCAAATGTAATGATAGCAAAGCCATCG | | |
| | TGAAGACCCTTGCTGCTACCGGGACAGGATTTGACTGTGCTAGCAAGACTGAAATACA | | |
| | GTTGGTGCAGAGTCTGGGGGTGCCTCCAGAGAGGATTATCTATGCAAATCCTTGTAAA | | |
| | CAAGTATCTCAAATTAACTATGCTGCTAATAATGGAGTCCAGATGATGACTTTTGATA | | |
| | GTGAAGTTGAGTTGATGAAAGTTGCCAGAGCACATCCCAAAGCAAAGTTGGTTTTGCG | | |
| | GATTGCCACTGATGATTCCAAAGCAGTCTGTCGTCTCAGTGTGAAATTCGGTGCCACG | | |
| | CTCAGAACCAGCAGGCTCCTTTTGGAACGGGCGAAAGAGCTAAATATCGATGTTGTTG | | |
| | GTGTCAGCTTCCATGTAGGAAGCGGCTGTACCGATCCTGAGACCTTCGTGCAGGCAAT | | |
| | CTCTGATGCCCGCTGTGTTTTTGACATGGGGGCTGAGGTTGGTTTCAGCATGTATCTG | | |
| | CTTGATATTGGCGGTGGCTTTCCTGGATCTCAGGATGTGAAACTTAAATTTGAAGAGA | | |
| | TCACCGGCGTAATCAACCCAGCGTTGGACAAATACTTTCCGTCAGACTCTGGAGTGAG | | |
| | AATCATAGCTGAGCCCGGCAGATACTATGTTGCATCAGCTTTCACGCTTGCAGTTAAT | | |
| | ATCATTGCCAAGAAAATTGTATTAAAGGAACAGACGGGCTCTGATGACGAAGATGAGT | | |
| | CGAGTGAGCAGACCTTTATGTATTATGTGAATGATGGCGTCTATGGATCATTTAATTG | | |
| | CATACTCTATGACCACGCACATGTAAAGCCCCTTCTGCAAAAGAGACCTAAACCAGAT | | |
| | GAGAAGTATTATTCATCCAGCATATGGGGACCAACATGTGATGGCCTCGATCGGATTG | | |
| | TTGAGCGCTGTGACCTGCCTGAAATGCATGTGGGTGATTGGATGCTCTTTGAAAACAT | | |
| | GGGCGCTTACACTGTTGCTGCTGCCTCTACGTTCAATGGCTTCCAGAGGCCGACGATC | | |
| | TACTATGTGATGTCAGGGCCTGCGTGGCAACTCATGCAGCAATTCCAGAACCCTGACT | | |
| | TCCCACCCGAAGTAGAGGAACAGGATGCCAGCACCCTGCCTGTGTCTTGTGCCTGGGA | | |
| | GAGTGGGATGAAACGCCACAGAGCAGCCTGTGCTTCGGCTAGTATTAATGTGCACCAT | | |
| | CACCACCATCACTGA | | |
| | ORF Start: at 1 | | ORF Stop: TGA at 1405 |
| | SEQ ID NO:134 | 468 aa | MW at 52071.6 kD |
| NOV10m, | TMNNFGNEEFDCHFLDEGFTAKDILDQKINEVSSSDDKDAFYVADLGDILKKHLRWLK | | |
| CG124907-04 | | | |
| Protein Sequence | ALPRVTPFYAVKCNDSKAIVKTLAATGTGFDCASKTEIQLVQSLGVPPERIIYANPCK | | |
| | QVSQIKYAANNGVQMMTFDSEVELMKVARAHPKAKLVLRIATDDSKAVCRLSVKFGAT | | |
| | LRTSRLLLERAKELNIDVVGVSFHVGSGCTDPETFVQAISDARCVFDMGAEVGFSMYL | | |
| | LDIGGGFPGSEDVKLKFEEITGVINPALDKYFPSDSGVRIIAEPGRYYVASAFTLAVN | | |
| | IIAKKTVLKEQTGSDDEDESSEQTFMYYVNDGVYGSFNCILYDHAHVKPLLQKRPKPD | | |
| | EKYYSSSIWGPTCDGLDRIVERCDLPEMHVGDWMLFENMGAYTVAAASTFNGFQRPTI | | |
| | YYVMSGPAWQLMQQFQNPDFPPEVEEQDASTLPVSCAWESGMKRHRAACASASINVHH | | |
| | HHHH | | |
| | SEQ ID NO:135 | 1305 bp | |
| NOV10n, | ACATCATCACCACCATCAAACAACTTTGGTAATGAAGAGTTTGACTGCCACTTCCTCG | | |
| CG124907-05 | | | |
| DNA Sequence | ATGAAGGTTTTACTGCCAAGGACATTCTGGACCAGAAAATTAATGAAGTTTCTTCTTC | | |
| | TGATGATAAGGATGCCTTCTATGTGGCAGACCTGGGAGACATTCTAAAGAAACATCTG | | |
| | AGGTGGTTAAAAGCTCTCCCTCGTGTCACCCCCTTTTATGCAGTCAAATGTAATGATA | | |
| | GCAAAGCCATCGTGAAGACCCTTGCTGCTACCGGGACAGGATTTGACTGTGCTAGCAA | | |
| | GACTGAAATACAGTTGGTGCAGAGTCTGGGGGTGCCTCCAGAGAGGATTATCTATGCA | | |
| | AATCCTTGTAAACAAGTATCTCAAATTAAGTATGCTGCTAATAATGGAGTCCACATCA | | |
| | TGACTTTTGATAGTGAAGTTGAGTTGATGAAAGTTGCCAGAGCACATCCCAAAGCAAA | | |
| | GTTGGTTTTGCGGATTGCCACTGATGATTCCAAAGCAGTCTGTCGTCTCAGTGTGAAA | | |
| | TTCGGTGCCACGCTCAGAACCAGCAGGCTCCTTTTGGAACGGGCGAAAGAGCTAAATA | | |
| | TCGATGTTGTTGGTGTCAGCTTCCATGTAGGAAGCGGCTGTACCGATCCTGAGACCTT | | |
| | CGTGCAGGCAATCTCTGATGCCCGCTGTGTTTTTGACATGGGGGCTGAGGTTGGTTTC | | |
| | AGCATGTATCTGCTTGATATTGGCGGTGGCTTTCCTGGATCTGAGGATGTGAAACTTA | | |
| | AATTTGAAGACATCACCGGCGTAATCAACCCAGCGTTGGACAAATACTTTCCGTCAGA | | |
| | CTCTGGAGTGAGAATCATAGCTGAGCCCGGCAGATACTATGTTGCATCAGCTTTCACG | | |
| | CTTGCAGTTAATATCATTGCCAAGAAAATTGTATTAAAGGAACAGACGGGCTCTGATG | | |
| | ACGAAGATGAGTCGAGTGAGCAGACCTTTATGTATTATGTGAATGATGGCGTCTATGG | | |
| | ATCATTTAATTGCATACTCTATGACCACGCACATGTAAAGCCCCTTCTGCAAAAGAGA | | |
| | CCTAAACCAGATGAGAAGTATTATTCATCCAGCATATGGGGACCAACATGTGATGGCC | | |
| | TCGATCGGATTGTTGAGCGCTGTGACCTGCCTGAAATGCATGTGGGTGATTGGATGCT | | |
| | CTTTGAAAACATCGGCGCTTACACTGTTGCTGCTGCCTCTACGTTCAATGGCTTCCAG | | |
| | AGGCCGACGATCTACTATGTGATGTCAGGGCCTGCGTGGCAACTCATGCAGTAGGCGG | | |
| | CCGCACTCGAGCACCACCACCACCACCAC | | |
| | ORF Start: at 1 | | ORF Stop: TAG at 1270 |
| | SEQID NO:136 | 423 aa | MW at 46885.9 kD |
| NOV10n, | TSSPPSNNFGNEEFDCHFLDEGFTAKDILDQKINEVSSSDDKDAFYVADLGDILKKHL | | |
| CG124907-05 | | | |
| Protein Sequence | RWLKALPRVTPFYAVKCNDSKAIVKTLAATGTGFDCASKTEIQLVQSLGVPPERIIYA | | |
| | NPCKQVSQIKYAANNGVQMMTFDSEVELMKVARAHPKAKLVLRIATDDSKAVCRLSVK | | |
| | FGATLRTSRLLLERAKELNIDVVGVSFHVGSGCTDPETFVQAISDARCVFDMGAEVGF | | |
| | SMYLLDIGGGFPGSEDVKLKFEEITGVINPALDKYFPSDSGVRIIAEPGRYYVASAFT | | |
| | LAVNIIAKKIVLKEQTGSDDEDESSEQTFMYYVNDGVYGSFNCILYDHAHVKPLLQKR | | |
| | PKPDEKYYSSSIWGPTCDGLDRIVERCDLPEMHVGDWMLFENMGAYTVAAASTFNGFQ | | |
| | RPTIYYVMSGPAWQLMQ | | |
| | SEQ ID NO:137 | 1305 bp | |
| NOV10o, | CACCATCACCACCATCACAACAACTTTGGTAATGAAGAGTTTGACTGCCACTTCCTCG | | |
| CG124907-06 | | | |
| DNA Sequence | ATGAAGGTTTTACTGCCAAGGACATTCTGGACCAGAAAATTAATGAAGTTTCTTCTTC | | |
| | TGATGATAAGGATGCCTTCTATGTGGCAGACCTGCGAGACATTCTAAAGAAACATCTG | | |
| | AGGTGGTTAAAAGCTCTCCCTCGTGTCACCCCCTTTTATGCAGTCAAATGTAATGATA | | |
| | GCAAAGCCATCGTGAAGACCCTTGCTGCTACCGGGACAGGATTTGACTGTGCTAGCAA | | |
| | GACTGAAATACAGTTGGTGCAGAGTCTGGGGGTGCCTCCAGAGAGGATTATCTATGCA | | |
| | AATCCTTGTAAACAAGTATCTCAAATTAAGTATGCTGCTAATAATGGAGTCCAGATGA | | |
| | TGACTTTTGATAGTGAAGTTGAGTTGATGAAAGTTGCCAGAGCACATCCCAAAGCAAA | | |
| | GTTGGTTTTGCGGATTGCCACTGATGATTCCAAAGCAGTCTGTCGTCTCAGTGTGAAA | | |
| | TTCGGTGCCACGCTCAGAACCAGCAGGCTCCTTTTGGAACGGGCGAAAGAGCTAAATA | | |
| | TCGATGTTGTTGGTGTCAGCTTCCATGTAGGAAGCGGCTGTACCGATCCTGAGACCTT | | |
| | CGTGCAGGCAATCTCTGATGCCCGCTGTGTTTTTGACATGGGGGCTGAGGTTGGTTTC | | |
| | AGCATGTATCTGCTTGATATTGGCGGTGGCTTTCCTGGATCTGAGGATGTGAAACTTA | | |
| | AATTTGAAGAGATCACCGGCGTAATCAACCCAGCGTTGGACAAATACTTTCCGTCAGA | | |
| | CTCTGGAGTGAGAATCATAGCTGAGCCCGGCAGATACTATGTTGCATCAGCTTTCACG | | |
| | CTTGCAGTTAATATCATTGCCAAGAAAATTGTATTAAAGGAACAGACGGGCTCTGATG | | |
| | ACGAAGATGAGTCGAGTGAGCAGACCTTTATGTATTATGTGAATGATGGCGTCTATGG | | |
| | ATCATTTAATTGCATACTCTATGACCACGCACATGTAAAGCCCCTTCTGCAAAAGAGA | | |
| | CCTAAACCAGATGAGAAGTATTATTCATCCAGCATATGGGGACCAACATGTGATGGCC | | |
| | TCGATCGGATTGTTGAGCGCTGTGACCTGCCTGAAATGCATGTGGGTGATTGGATGCT | | |
| | CTTTGAAAACATGGGCGCTTACACTGTTGCTGCTGCCTCTACGTTCAATGGCTTCCAG | | |
| | AGGCCGACGATCTACTATGTGATGTCAGGGCCTGCGTGGCAACTCATGCAGTAGGCGG | | |
| | CCGCACTCGAGCACCACCACCACCACCAC | | |
| | ORF Start: at 19 | | ORF Stop: TAG at 1270 |
| | SEQ ID NO:138 | 417 aa | MW at 46329.3 kD |

| | |
|---|---|
| NOV10o, | NNFGNEEFDCHFLDEGFTAKDILDQKINEVSSSDDKDAFYVADLGDILKKHLRWLKAL |
| CG124907-06 | |
| Protein Sequence | PRVTPFYAVKCNDSKAIVKTLAATGTGFDCASKTEIQLVQSLGVPPERIIYANPCKQV |
| | SQIKYAANNGVQMMTFDSEVELMKVARAHPKAKLVLRIATDDSKAVCRLSVKFGATLR |
| | TSRLLLERAKELNIDVVGVSFHVGSGCTDPETFVQAISDARCVFDMGAEVGFSMYLLD |
| | IGGGFPGSEDVKLKFEEITGVINPALDKYFPSDSGVRIIAEPGRYYVASAFTLAVNII |
| | AKKIVLKEQTGSDDEDESSEQTFMYYVNDGVYGSFNCILYDHAHVKPLLQKRPKPDEK |
| YYSSSIWGPTCDGLDRIVERCDLPEMHVGDWMLFENMGAYTVAAASTFNGFQRPTIYY | |
| | VMSGPAWQLMQ |

Sequence comparison of the above protein sequences yields the following sequence relationships shown in Table 10B.

TABLE 10B
Comparison of NOV10a against NOV10b through NOV10o.

| | NOV10a | Identities/ |
|---|---|---|
| | Residues/ | Similarities |
| | Match | for the |
| Protein Sequence | Residues | Matched Region |
| NOV10b | 1 . . . 461 | 461/461 (100%) |
| | 1 . . . 461 | 461/461 (100%) |
| NOV10c | 1 . . . 461 | 461/461 (100%) |
| | 5 . . . 465 | 461/461 (100%) |
| NOV10d | 2 . . . 461 | 460/460 (100%) |
| | 10 . . . 469 | 460/460 (100%) |
| NOV10e | 1 . . . 461 | 461/461 (100%) |
| | 2 . . . 462 | 461/461 (100%) |
| NOV10f | 2 . . . 461 | 460/460 (100%) |
| | 7 . . . 466 | 460/460 (100%) |
| NOV10g | 2 . . . 418 | 417/417 (100%) |
| | 7 . . . 423 | 417/417 (100%) |
| NOV10h | 1 . . . 461 | 461/461 (100%) |
| | 2 . . . 462 | 461/461 (100%) |
| NOV10i | 1 . . . 461 | 461/461 (100%) |
| | 1 . . . 461 | 461/461 (100%) |
| NOV10j | 2 . . . 418 | 417/417 (100%) |
| | 7 . . . 423 | 417/417 (100%) |
| NOV10k | 1 . . . 461 | 461/461 (100%) |
| | 1 . . . 461 | 461/461 (100%) |
| NOV10l | 2 . . . 461 | 460/460 (100%) |
| | 10 . . . 469 | 460/460 (100%) |
| NOV10m | 1 . . . 461 | 461/461 (100%) |
| | 2 . . . 462 | 461/461 (100%) |
| NOV10n | 2 . . . 418 | 417/417 (100%) |
| | 7 . . . 423 | 417/417 (100%) |
| NOV10o | 2 . . . 418 | 417/417 (100%) |
| | 1 . . . 417 | 417/417 (100%) |

Further analysis of the NOV10a protein yielded the following properties shown in Table 10C.

TABLE 10C
Protein Sequence Properties NOV10a

| | |
|---|---|
| PSort | 0.6000 probability located in nucleus; |
| analysis: | 0.3922 probability located in microbody |
| | (peroxisome); 0.1000 probability located |
| | in mitochondrial matrix space; 0.1000 |
| | probability located in lysosome (lumen) |
| SignalP | No Known Signal Sequence Predicted |
| analysis: | |

A search of the NOV10a protein against the Geneseq database, a proprietary database that contains sequences published in patents and patent publication, yielded several homologous proteins shown in Table 10D.

TABLE 10D
Geneseq Results for NOV10a

| | | NOV10a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG73867 | Human colon cancer antigen | 1 . . . 461 | 461/461 (100%) | 0.0 |
| | protein SEQ ID NO: 4631 - Homo | 6 . . . 466 | 461/461 (100%) | |
| | sapiens, 466 aa. [WO200122920- | | | |
| | A2, 05 APR. 2001] | | | |
| AAB58391 | Lung cancer associated polypeptide | 1 . . . 461 | 461/461 (100%) | 0.0 |
| | sequence SEQ ID 729 - Homo | 6 . . . 466 | 461/461 (100%) | |
| | sapiens, 466 aa. [WO200055180- | | | |
| | A2, 21 SEP. 2000] | | | |
| AAR37270 | ODC - Synthetic, 461 aa. | 1 . . . 461 | 460/461 (99%) | 0.0 |
| | [EP542287-A, 19 MAY 1993] | 1 . . . 461 | 461/461 (99%) | |
| AAB52181 | Human secreted protein BLAST | 17 . . . 444 | 427/428 (99%) | 0.0 |
| | search protein SEQ ID NO: 137 - | 1 . . . 428 | 428/428 (99%) | |
| | Homo sapiens, 428 aa. | | | |
| | [WO200061624-A1, 19 OCT. | | | |
| | 2000] | | | |
| AAW76000 | Ornithine decarboxylase amino | 1 . . . 461 | 417/461 (90%) | 0.0 |
| | acid sequence - Mus sp, 461 aa. | 1 . . . 461 | 434/461 (93%) | |
| | [US5811634-A, 22 SEP. 1998] | | | |

In a BLAST search of public sequence datbases, the NOV10a protein was found to have homology to the proteins shown in the BLASTP data in Table 10E.

TABLE 10E
Public BLASTP Results for NOV10a

| | | | Identities/ | |
|---|---|---|---|---|
| Protein | | | Similarities for | |
| Accession | | NOV10a Residues/ | the Matched | Expect |
| Number | Protein/Organism/Length | Match Residues | Portion | Value |
| P11926 | Ornithine decarboxylase (EC | 1 . . . 461 | 461/461 (100%) | 0.0 |
| | 4.1.1.17) (ODC) - Homo sapiens | 1 . . . 461 | 461/461 (100%) | |
| | (Human), 461 aa. | | | |
| P27117 | Ornithine decarboxylase (EC | 1 . . . 461 | 431/461 (93%) | 0.0 |
| | 4.1.1.17) (ODC) - Bos taurus | 1 . . . 461 | 444/461 (95%) | |
| | (Bovine), 461 aa. | | | |
| P09057 | Ornithine decarboxylase (EC | 1 . . . 461 | 422/461 (91%) | 0.0 |
| | 4.1.1.17) (ODC) - Rattus | 1 . . . 461 | 434/461 (93%) | |
| | norvegicus (Rat), 461 aa. | | | |
| P27119 | Ornithine decarboxylase (EC | 1 . . . 461 | 421/461 (91%) | 0.0 |
| | 4.1.1.17) (ODC) - Mus pahari | 1 . . . 461 | 436/461 (94%) | |
| | (Shrew mouse), 461 aa. | | | |
| P00860 | Ornithine decarboxylase (EC | 1 . . . 461 | 417/461 (90%) | 0.0 |
| | 4.1.1.17) (ODC) - Mus musculus | 1 . . . 461 | 434/461 (93%) | |
| | (Mouse), 461 aa. | | | |

PFam analysis predicts that the NOV10a protein contains the domains shown in the Table 10F.

TABLE 10F
Domain Analysis of NOV10a

| | | Identities/ | |
|---|---|---|---|
| | NOV10a | Similarities | |
| | Match | for the | Expect |
| Pfam Domain | Region | Matched Region | Value |
| Orn_Arg_deC_N | 44 . . . 282 | 131/289 (45%) | 7.8e−132 |
| | | 225/289 (78%) | |
| Orn_DAP_Arg_deC | 285 . . . 409 | 68/199 (34%) | 5.6e−62 |
| | | 119/199 (60%) | |

### Example 11

The NOV11 clone was analyzed, and the nucleotide and encoded polypeptide sequences are shown in Table 11A.

TABLE 11A
NOV11 Sequence Analysis

| | |
|---|---|
| | SEQ ID NO: 139 994 bp |
| NOV11a, | CACACAAGTCCGCCTATGTACTCTCTGGATCGAATATTTGCTGGATTTCGAACACGAA |
| CG128347-01 | |
| DNA Sequence | GTCAGATGCTGTTGGGTCACATAGAAGAACAAGATAAGGTCCTCCACTGCCAATTTTC |
| | TGATAACAGTGATGATGAAGAATCAGAAGGCCAAGAGAAATCTGGAACTAGGTGTAGA |
| | AGTCGTTCATGGATTCAGAAGCCAGACTCTGTTTGTTCCCTTGTTGAATTGAGTGATA |
| | CTCAGGATGAAACACAAAAGTCAGATTTGGAGAATGAAGATTTAAAGATTGATTGTCT |
| | CCAGGAGAGTCAAGAATTGAATTTGCAAAAATTAAAGAATTCAGAACGCATACTTACT |
| | GAAGCTAAACAAAAAATGAGAGAACTTACAATTAACATCAAGATGAAGGAAGATCTGA |
| | TTAAAGAATTAATAAAAACAGGTAATGATGCCAAGTCTGTAAGCAAGCAGTATTCTTT |
| | GAAAGTAACAAAGCTAGAGCATGATGCAGAACAGGCAAAAGTCGAACTAACTGAAACA |
| | CAAAAGCAGCTACAGGAGCTGGAAAACAAAGATCTTTCTGATGTTGCAATGAACGTAA |
| | AATTACAGAAAGAGTTTCGTAAAAAGATGGATGCTGCAAAGCTGAGAGTTCAGGTCTT |
| | ACAGAAGAAGCAACAAGATAGTAAGAAACTGGCATCACTGTCAATCCAAAATGAGAAA |
| | CGTGCTAATGAACTAGAGCAGAGTGTAGATCACATGAAATATCAAAAGATACAGCTAC |
| | AAAGAAAACTACAAGAAGAAAATGAAAAAAGGAAGCAACTGGATGCAGTAATTAAGCG |
| | GGACCAGCAAAAAATCAAAGTAATATTGTCATACATTCCTGCTAAGTATAATATGAAA |
| | TGTTAAACGGCTCAGAGCTAACGAATCCATGGTCTTCATTCAGTTGGCTTGTGAAGTA |
| | TCTATCCTTGACTTGCCCTTCACTGCTGTCCTTATTCACTTTAAAGCTTTGTTCATCT |
| | ACATAGTA |
| | ORF Start: ATG at 16 ORF Stop: TAA at 874 |
| | SEQ ID NO: 140 286 aa MW at 33507.0kD |
| NOV11a, | MYSLDRIFAGFRTRSQMLLGHIEEQDKVLHCQFSDNSDDEESEGQEKSGTRCRSRSWI |
| CG128347-01 | |
| Protein Sequence | QKPDSVCSLVELSDTQDETQKSDLENEDLKIDCLQESQELNLQKLKNSERILTEAKQK |
| | MRELTINIKNKEDLIKELIKTGNDAKSVSKQYSLKVTKLEHDAEQAKVELTETQKQLQ |
| | ELENKDLSDVAMKVKLQKEFRKKNDAAKLRVQVLQKKQQDSKKLASLSIQNEKRANEL |
| | EQSVDHMKYQKIQLQRKLQEENEKRKQLDAVIKRDQQKIKVILSYIPAKYNMKC |
| | SEQ ID NO: 141 4622 bp |
| NOV11b, | AGGAGTCCAGCGCTCGCCGACAGGGGCCTGGGCTGTCCCGAGCCGGAATCCAGATCTT |
| CG128347-02 | |
| DNA Sequence | ACATAAGATGGAAGTCTCTCACACTAGATACTGAACATTAAATAGAAAATCTATTTAG |
| | TAAAATCTAAGTTGCCATGGAAGAAATACCAGTAAAAGTTGCTGTAAGAATTAGACCT |
| | CTGCTTTGCAAAGAAGCTCTTCATAATCATCAAGTTTGTGTGAGAGTTATTCCAAACA |
| | GCCAGCAAGTTATCATTGGGAGAGATAGAGTCTTCACTTTTGATTTTGTTTTTGGCAA |
| | AAATTCCACTCAAGATGAAGTTTATAACACATGTATAAAGCCCCTAGTGTTGTCACTC |
| | ATTGAGGGCTATAATGCAACTGTTTTTGCCTATGGACAAACTGGATCTGGGAAGAcAT |
| | ACACCATTGGAGGGGGCCATATTGCTTCAGTTGTGGAGGGCCAAAAGGGTATCATTCC |
| | TCGAGCTATTCAAGAAATATTTCAAAGCATCTCTGAACATCCTAGCATTGACTTTAAT |
| | GTAAAAGTATCTTATATAGAAGTGTACAAGGAAGACCTAAGAGATCTTCTAGAATTGG |
| | AGACATCCATGAACGATCTTCACATCCGAGAAGATGAAAAAGGAAACACAGTGATTGT |
| | TGGGGCCAAGGAATGCCATGTGGAGAGTGCACGTGAAGTGATGAGTCTTTTGGAGATG |
| | GGGAATGCAGCCAGACATACAGGTACCACTCAAATGAATGAGCACTCCAGCAGATCAC |
| | ATGCAATTTTTACAATCAGCATTTGTCAAGTTCATAAAAATATGGAGGCAGCTGAAGA |
| | TGGATCATGGTATTCCCCTCGGCATATTGTCTCAAAGTTCCACTTTGTGGATTTGGCA |
| | GGATCAGAAAGAGTAACCAAAACGGGGAATACTGGTGAACGGTTCAAAGAATCCATTC |
| | AAATCAATAGTGGATTGCTGGCTTTAGGAAATGTAATAAGCGCTCTTGGGGACCCACG |
| | CAGGAAGAGTTCACATATTCCATATAGGGATGCTAAAATTACCCGGCTTCTGAAAGAT |
| | TCTCTGGGAGGCAGTGCTAAGACTGTCATGATCACATGTGTCAGCCCCTCCTCCTCGA |
| | ATTTTGATGAGTCCTTAAATTCTCTCAAATATGCCAACAGAGCACGGAACATTAGAAA |
| | CAAACCCACTGTAAACTTCAGCCCCGAGTCAGACCGTATAGATGAAATGGAATTTGAG |
| | ATTAAATTGCTTCGAGAAGCTTTGCAAAGCCAGCAGGCTGGTGTCAGCCAAACTACCC |
| | AGATCAATCGAGAAGGGAGTCCTGATACAAATAGGATTCATTCTCTTGAGGAGCAAGT |
| | AGCTCAGCTTCAAGGAGAATGTCTGGGTTACCAGTGTTGTGTAGAAGAAGCCTTTACC |
| | TTCCTGGTTGACCTAAAAGATACTGTCAGACTAAACGAAAAGCAGCAACACAAACTGC |
| | AGGAGTGGTTTAACATGATCCAAGAGGTCAGGAAGGCTGTCCTCACCTCATTTCGAGG |
| | AATCGGAGGCACTGCAAGTCTGGAAGAAGGACCACAGCATGTTACAGTTCTCCAGCTG |
| | AACAGAGAGCTTAAGAAATGCCAGTGTGTGCTTGCTGCTGATGAAGTAGTATTTAATC |
| | AGAAGGAACTGGAGGTGAAGGAACTGAAGAATCAAGTGCAGATGATGGTACAGGAAAA |
| | CAAAGGGCATGCTGTATCTTTGAAAGAAGCGCAAAAAGTGAATAGACTGCAGAATGAA |
| | AAAATAATAGAACAACAACTTCTTGTGGATCAACTGAGTGAAGAACTAACAAAACTTA |
| | ACCTGTCAGTGACTTCTTCAGCTAAAGAAAATTGTGGAGATGGGCCAGATGCCAGGAT |
| | CCCTGAAAGGAGACCATATACTGTACCATTTGATACTCATTTGGGGCATTATATTTAT |
| | ATCCCATCAAGACAAGATTCCAGGAAGGTCCACACAAGTCCGCCTATGTACTCTCTGG |
| | ATCGAATATTTGCTGGATTTCGAACACGAAGTCAGATGCTGTTGGGTCACATAGAAGA |
| | ACAAGATAAGGTCCTCCACTGCCAATTTTCTGATAACAGTGATGATGAAGAATCAGAA |
| | GGCCAAGACAAATCTGGAACTAGATGTAGAAGTCGTTCATGGATTCAGAAGCCAGACT |
| | CTGTTTGTTCCCTTGTTGAATTGAGTGATACTCAGGATGAAACACAAAACTCAGATTT |
| | GGAGAATGAAGATTTAAGATTGATTGTCTCCAGGAGAGTCAAGAATTGAATTTGCAA |
| | AAATTAAAGAATTCAGAACGCATACTTACTGAAGCTAAACAAAAAATGAGAGAACTTA |
| | CAATTAACATCAACATGAAGGAAGATCTGATTAAAGAATTAATAAAAACAGGTAATGA |
| | TGCCAAGTCTGTAAGCAACCAGTATTCTTTGAAAGTAACAAAGCTAGAGCATGATGCA |
| | GAACAGGCAAAAGTCGAACTGATTGAAACACAAAAGCAGCTACAGGAGCTGGAAAACA |
| | AAGATCTTTCTGATGTTGCAATGAAGGTAAAATTACAGAAAGAGTTTCGTAAAAAGAT |
| | GGATGCTGCAAAGCTGAGAGTTCAGGTCTTGCAGAAGAAGCAACAAGATAGTAAGAAA |
| | CTGGCATCACTGTCAATCCAAAATGAGAAACGTGCTAATGAGCTAGAGCAGAGTGTAG |
| | ATCACATGAAATATCAAAAGATACAGCTACAAAGAAAACTACGAGAAGAAAATGAAAA |
| | AAGGAAGCAACTGGATGCAGTAATTAAGCGGGACCAGCAAAAAATCAAAGTAATACAA |
| | TTAAAAACAGGACAGGAAGAAGGTCTAAAACCGAAAGCTGAGGACCTTGATGCATGTA |
| | ACTTGAAAAGCAGAAAAGGTTCGTTTGGAAGTATAGACCATCTCCAGAAATTGGATGA |
| | GCAAAAGAAATGGTTAGATGAAGAAGTAGAGAAAGTTCTGAACCAACGCCAAGAATTA |
| | GAGGAGCTGGAAGCAGACTTAAAGAAACGGGAGGCCATAGTTTCTAAGAAGGAGGCTC |
| | TGTTACAGGAGAAGAGTCACCTGGAAATAAGAAATTGAGATCTAGTCAGGCCTTAAA |
| | CACAGATAGTTTGAAAATATCAACTCGCCTGAACTTACTGGAACAAGAGTTGTCTGAA |
| | AAGAATGTGCAGCTCCAGACCAGTACAGCTGAGGAGAAAACAAAGATTTCAGAACAAG |
| | TTGAAGTCCTCCAGAAAGAAAAGGATCAGCTCCAGAAACGCAGACACGATGTGGATGA |
| | AAAACTTAAAAATGGTAGAGTGTTATCACCTGAAGAAGAACATGTTCTTTTCCAACTT |
| | GAAGAAGGGATAGAAGCTTTGGAAGCTGCAATTGAATACAGGAATGAAAGTATCCAGA |
| | ATCGCCAGAAGTCACTTAGAGCATCATTCCATAACCTCTCTCGTGGTGAAGCAAATGT |
| | CTTGGAAAAGCTAGCTTGCCTGAGTCCTGTTGAGATTACAACTATTCTTTTCAGATAT |
| | TTCAATAAGGTGGTGAATTTGCGAGAAGCTGAACGGAACAACAGTTATATAATGAAG |
| | AAATGAAAATGAAAGTTCTGGAACGGGATAATATGGTTCGTGAATTACAATCTGCACT |
| | GGACCATCTAAAATTGCAGTGTGACCGGAGACTGACCCTCCAGCAAAAGGAACACGAA |
| | CAAAAGATGCAGTTGCTATTACATCATTTCAAAGAACAAGATGGAGAAGGCATTATGG |
| | AAACTTTCAAAACATATGAAGATAAAATCCAGCAGTTGGAAAAAGATCTTTATTTCTA |
| | TAAGAAAACCAGCCGGGATCATAAGAAGAAACTTAAGGAACTGGTAGGGGAAGCAATT |
| | CGGCGGCAACTAGCATCATCAGAGTATCAAGAGGCTGGAGATGGAGTCCTGAAGCCAG |
| | AAGGAGGAGGCATGCTTTCAGAAGAATTAAAATGGGCATCCAGACCTGAAAGTATGAA |
| | ATTAAGTGGAAGAGAAAGAGAATGGACAGTTCAGCAAGCAGCTTAAGAACACAGCCA |
| | AATCCTCAAAAGCTCTGGGAAGATATCCCAGAATTACCTCCAATTCATAGTTCTTTAG |
| | CACCCCCCAGTGGGCATATGTTAGGTAATGAGAATAAAACAGAAACAGATGATAATCA |
| | GTTTACAAAATCTCACAGTCGACTGTCATCCCAAATTCAGGTTGTGGGAAATGTGGGA |
| | CGACTTCATGGTGTCACACCTGTAAAACTGTGTCGAAAAGAATTACGTCAAATTTCCG |
| | CCTTGGAACTATCATTGCGACGTTCCAGTCTTGGAGTTGGCATTGGATCAATGGCTGC |
| | TGATTCCATCGAAGTATCTAGGAAACCAAGGGACTTAAAAACTTAGACATTGAATAAT |
| | AGAACTTTTAGTAGATATGTAAAAAGATTCCTTTTTCTAACCTGTTAAAAACTAAAGC |
| | TCAAGTTCACTACCTCTTTCCTCAGAATAAAGGAAGAAGGGGAGCAAGGAATCCCTAA |
| | TTCTTTTATATGCTATAGATGTGTACATCTTCTATATATATTTGCGGAGTTTTAGTTT |
| | ATATTCCCATAGTAATCAAACATGTTTTCCAATACTTGATAACATTTAAATATTTATA |
| | AATACGCTTAAATGTTTTTCCAGGCATATTTGAAGATTAA |
| | ORF Start: ATG at 133 ORF Stop: TAG at 4336 |
| | SEQ ID NO: 142 1401 aa MW at 16O242.6kD |
| NOV11b, | MEEIPVKVAVRIRPLLCKEALHNHQVCVRVIPNSQQVTIGRDRVFTFDFVFGKNSTQD |
| CG128347-02 | |
| Protein Sequence | EVYNTCIKPLVLSLIEGYNATVFAYGQTGSGKTYTIGGGHIASVVEGQKGTIPRAIQE |
| | IFQSISEHPSIDFNVKVSYIEVYKEDLRDLLELETSMKDLHIREDEKGNTVIVGAKEC |
| | HVESAGEVMSLLEMGNAARHTGTTQMNEHSSRSHAIFTISICQVHKNMEAAEDGSWYS |
| | PRHIVSKFHFVDLAGSERVTKTGNTGERFKESIQIMSCLLALGNVISALCDPRRKSSH |
| | IPYRDAKITRLLKDSLGGSAKTVMITCVSPSSSNFDESLNSLKYANRARNIRNKPTVN |
| | FSPESDRIDEMEFEIKLLREALQSQQAGVSQTTQINREGSPDTNRIHSLEEQVAQLQG |
| | ECLGYQCCVEEAFTFLVDLKDTVRLNEKQQHKLQEWFNMIQEVRKAVLTSFRGIGGTA |
| | SLEEGPQHVTVLQLKRELKKCQCVLAADEVVFNQKELEVKELKNQVQMMVQENKGHAV |
| | SLKEAQKVNRLQNEKTIEQQLLVDQLSEELTKLNLSVTSSAKENCGDGPDARIPERRP |
| | YTVPFDTHLGHYIYIPSRQDSRKVHTSPPMYSLDRIFAGFRTRSQMLLGHIEEQDKVL |
| | HCQFSDNSDDEESEGQEKSGTRCRSRSWIQKPDSVCSLVELSDTQDETQKSDLENEDL |
| | KIDCLQESQELNLQKLKNSERILTEAKQKMRELTINJKMKEDLIKELIKTGNDAKSVS |
| | KQYSLKVTKLEHDAEQAKVELIETQKQLQELENKDLSDVAMKVKLQKEFRKKMDAAKL |
| | RVQVLQKKQQDSKKLASLSIQNEKRANELEQSVDHMKYQKIQLQRTCIJREENEKRKQLD |
| | AVIKRDQQKIKVIQLKTGQEEGLKPKAEDLDACNLKRRKGSFGSIDHLQKLDEQKKWL |
| | DEEVEKVLNQRQELEELEADLKKREAIVSKKEALLQEKSHLENKKLRSSQALNTDSLK |
| | ISTRLNLLEQELSEKNVQLQTSTAEEKTKISEQVEVLQKEKDQLQKRRHDVDEKLKNG |
| | RVLSPEEEHVLFQLEEGIEALEAAIEYRNESIQNRQKSLRASFHNLSRGEANVLEKLA |
| | CLSPVEIRTILFRYFNKVVNLREAERKQQLYNEEMKMKVLERDNMVRELESALDHLKL |
| | QCDRRLTLQQKEHEQKMQLLLHHFKEQDGEGIMETFKTYEDKIQQLEKDLYFYKKTSR |
| | DHKKKLKELVGEAIRRQLASSEYQEAGDGVLKPEGGGMLSEELKWASRPESMKLSGRE |
| | REMDSSASSLRTQPNPQKLWEDIPELPPIHSSLAPPSGHMLGNENKTETDDNQFTKSH |
| | SRLSSQIQVVGNVGRLHGVTPVKLCRKELRQISALELSLRRSSLGVGIGSMAADSIEV |
| | SRKPRDLKT |

Sequence comparison of the above protein sequences yields the following sequence relationships shown in Table 11B.

TABLE 11B
Comparison of NOV11a against NOV11b.

| | NOV11a | Identities/ |
|---|---|---|
| | Residues/ | Similarities |
| Protein | Match | for the |
| Sequence | Residues | Matched Region |
| NOV11b | 1 . . . 274 | 272/274 (99%) |
| | 610 . . . 883 | 273/274 (99%) |

Further analysis of the NOV11a protein yielded the following properties shown in Table 11C.

TABLE 11C
Protein Sequence Properties NOVlla

| | |
|---|---|
| PSort | 0.5517 probability located in mitochondrial |
| analysis: | matrix space: 0.3000 probability located in |
| | microbody (peroxisome); 0.2717 probability |
| | located in mitochondrial inner membrane; |
| | 0.2717 probability located in mitochondrial |
| | intermembrane space |
| SignalP | No Known Signal Sequence Predicted |
| analysis: | |

A search of the NOV11a protein against the Geneseq database, a proprietary database that contains sequences published in patents and patent publication, yielded several homologous proteins shown in Table 11D.

TABLE 11D
Geneseq Results for NOV11a

| | | NOV11a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAB42353 | Human ORFX ORF2117 | 1 . . . 274 | 270/274 (98%) | e−150 |
| | polypeptide sequence SEQ ID | 42 . . . 315 | 274/274 (99%) | |
| | NO: 4234 - Homo sapiens, 833 aa. | | | |
| | [WO200058473-A2, 05 OCT. 2000] | | | |
| ABB80078 | Human kinesin motor protein | 1 . . . 274 | 271/274 (98%) | e−149 |
| | (HsKrp5) amino acid sequence - | 488 . . . 761 | 272/274 (98%) | |
| | Homo sapiens, 1279 aa. | | | |
| | [US6379941-B1, 30 APR. 2002] | | | |
| AAM40604 | Human polypeptide SEQ ID NO | 55 . . . 286 | 219/232 (94%) | e−118 |
| | 5535 - Homo sapiens, 232 aa. | 1 . . . 232 | 226/232 (97%) | |
| | [WO200153312-A1, 26 JUL. 2001] | | | |
| AAM38818 | Human polypeptide SEQ ID NO | 64 . . . 286 | 218/223 (97%) | e−118 |
| | 1963 - Homo sapiens, 229 aa. | 7 . . . 229 | 222/223 (98%) | |
| | [WO200153312-A1, 26 JUL. 2001] | | | |
| AAY41675 | Human channel-related molecule | 64 . . . 286 | 218/223 (97%) | e−118 |
| | HCRM-3 - Homo sapiens, 229 aa. | 7 . . . 229 | 222/223 (98%) | |
| | [WO9943807-A2, 02 SEP. 1999] | | | |

In a BLAST search of public sequence datbases, the NOV11a protein was found to have homology to the proteins shown in the BLASTP data in Table 11E.

TABLE 11E
Public BLASTP Results for NOV11a

| | | NOVlla | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9UF54 | Hypothetical 96.7 kDa protein - | 1 . . . 274 | 265/274 (96%) | e−146 |
| | Homo sapiens (Human), 833 aa | 42 . . . 315 | 269/274 (97%) | |
| | (fragment). | | | |
| Q95LL1 | Hypothetical 98.5 kDa protein - | 1 . . . 256 | 245/256 (95%) | e−135 |
| | Macaca fascicularis (Crab eating | 610 . . . 865 | 254/256 (98%) | |
| | macaque) (Cynomolgus monkey), | | | |
| | 865 aa (fragment). | | | |
| Q95JP3 | Hypothetical 49.3 kDa protein - | 1 . . . 248 | 242/248 (97%) | e−132 |
| | Macaca fascicularis (Crab eating | 166 . . . 413 | 247/248 (99%) | |
| | macaque) (Cynomolgus monkey), | | | |
| | 428 aa. | | | |
| Q9QXL2 | Kif21a - Mus musculus (Mouse), | 23 . . . 270 | 68/255 (26%) | 2e−16 |
| | 1573 aa. | 551 . . . 793 | 129/255 (49%) | |
| Q64075 | Nucleoporin p62 homolog protein - | 90 . . . 239 | 55/151 (36%) | 6e−13 |
| | Rattus sp, 215 aa (fragment). | 12 . . . 151 | 86/151 (56%) | |

PFam analysis predicts that the NOV11a protein contains the domains shown in the Table 11F.

TABLE 11F
Domain Analysis of NOV11a

| | | | |
|---|---|---|---|
| | | Identities/ | |
| | NOV11a | Similarities | |
| Pfam | Match | for the | Expect |
| Domain | Region | Matched Region | Value |

No Significant Matches Found

### Example 12

The NOV12 clone was analyzed, and the nucleotide and encoded polypeptide sequences are shown in Table 12A.

TABLE 12A
NOV12 Sequence Analysis

| | |
|---|---|
| | SEQ ID NO: 143 2754 bp |
| NOV12a, | ATTGCCCCTGTAACCTGTCAAAGAAGAGCTAAGGGAGCTTTCGGGGTTGGCTTCTTGG |
| CG135823-01 | |
| DNA Sequence | AGGCTGCTTTCTCCTTTACTTGGAAGGCTTCGCTACTGATGGACCCATACATGATTCA |
| | GATGAGCAGCAAAGGCAACCTCCCCTCAATTCTGGACGTGCATGTCAACGTTGGTGGG |
| | AGAAGCTCTGTGCCGGGAAAAATGAAAGGCAGAAAGGCCAGGTGGTCTGTGAGGCCCT |
| | CAGACATGGCCAAGAAAACTTTCAACCCCATCCGAGCCATTGTGGACAACATGAAGGT |
| | GAAACCAAATCCAAACAAAACCATGATTTCCCTGTCCATTGGGGACCCTACTGTGTTT |
| | GGAAACCTGCCTACAGACCCTGAAGTTACCCAGGCAATGAAAGATGCCCTGGACTCGG |
| | GCAAATATAATGGCTATCCCCCATCCATCGGCTTCCTATCCAGTCGGGAGGAGATTGC |
| | TTCTTATTACCACTGTCCTGAGGCACCCCTAGAAGCTAAGGACGTCATTCTGACAAGT |
| | GGCTGCAGCCAAGCTATTGACCTTTGTTTAGCTGTGTTGGCCAACCCAGGGCAGAACA |
| | TCCTGGTTCCAAGACCTGGTTTCTCTCTCTACAAGACTCTGGCTGAGTCTATGGGAAT |
| | TGAGGTCAAACTCTACAATTTGTTGCCAGAGAAATCTTGGGAAATTGACCTGAAACAA |
| | CTGGAATATCTAATTGATGAAAAGACAGCTTGTCTCATTGTCAATAATCCATCAAACC |
| | CCTGTGGGTCAGTGTTCAGCAAACGTCATCTTCAGAAGATTCTGGCAGTGGCTGCACG |
| | GCAGTGTGTCCCCATCTTAGCTGATGAGATCTATGGAGACATGGTGTTTTCGGATTGC |
| | AAATATGAACCACTGGCCACCCTCAGCACCGATGTCCCCATCCTGTCCTGTGGAGGGC |
| | TGGCCAAGCGCTGGCTGGTTCCTGGCTGGAGGTTGGGCTGGATCCTCATTCATGACCG |
| | AAGAGACATTTTTGGCAATGAGATCCGAGATGGGCTGGTGAAGCTGAGTCAGCGCATT |
| | TTGGGACCCTGTACCATTGTCCAGGGACCTCTGAAAAGCATCCTATGTCGCACCCCGG |
| | GAGAGTTTTACCACAACACTCTGAGCTTCCTCAAGTCCAATGCTGATCTCTGTTATGG |
| | GGCGTTGGCTGCCATCCCTGGACTCCGGCCAGTCCGCCCTTCTGGGGCTATGTACCTC |
| | ATGGTTGGAATTGAGATGGAACATTTCCCAGAATTTGAGAACGATGTGGAGTTCACGG |
| | AGCGGTTAGTTGCTGAGCAGTCTGTCCACTGCCTCCCAGCAACGTCCTTTGAGTACCC |
| | GAATTTCATCCGAGTGGTCATCACAGTCCCCGAGGTGATGATGCTGGAGGCGTGCAGC |
| | CGGATCCAGGAGTTCTGTGAGCAGCACTACCATTGTGCTGAAGGCAGCCAGGAGGAGT |
| | GTGATAAATAGGCCTGCATCCATTCTCCTGAGGATGTGTCCCATCTAGGGAAGGCTGG |
| | ACTAGGCCTTGCGGCTCCTCAGGGACTCAGGTGGCCCTACTGGGAGAGGGGCCTCAAA |
| | TGCACCATGTCAAGGGTTCAAGATTGTTCCTGCTTTTCCCCAAGTACAACCACACCCA |
| | CACTCAGATCCTCCTCATTCACATCGCAGATTACTCCCTTGCTCTGCGCTGCTAGAGT |
| | GACTCACTAATTCATTAATCTGCCTCCCTCTCGTAAGATTTCCTTCTTTTTTTTCTTG |
| | AAAGTACCAGGTGAACAAAGTTTACCAGAAAGCAGTTGAGACAAGAAAATAAGAGCTC |
| | AGGATGAGGGAAAAGAAAAAGATTGAGAGAATTTGTGCCCCCAACCATTTCCTCAGAC |
| | TCTAAGAAAGAACACGCTCTCTCCAGGCAGGTCTGAAGCTCAACTCTCTTATTGCCTC |
| | ACTTCAGGTATACCTCACTTTACACAATAGAATTATAACTGGAAAGAAGTTGGGGACA |
| | CATGTATTTGGTGATTACATTTTAAACACATTAGGAAAAGTTGCTATTTGAACTTTTT |
| | ATTGATTTTTGGGGGGAGTAAAGAATTATTTTGGATGCAAATAAATATCCTTTAATTG |
| | ATCGACTTGCCAAATTTAGATTTGTGTGCATCAGGCTTTCTTTTTTTTCTTTTTTTAG |
| | AGAAGTTCAATATAAGCTTTTCTTTTCTTTGTTTCTTTCTTTCTTTATTTTGAGATGG |
| | AGTCTTGCTCTGTCGCCCATGCTGGAGTGCAGTGGCGCGATCTCGGCTCACTGCAACC |
| | TCCACCTCCTGGGTTCAAGCGATTCTCTTGCCTCAACCTCCCAAGCAGTTGGGACTAC |
| | AGGCGTGAGCCACCATGCCCCGCTAATTTTTGTATTTTTAGTAGAGACAGGGTTTCAC |
| | CATGTTAGCCAGGCTGGTCTCAAACTCCTGACCTCAGGCAATCTGCCCGCCTGGGTCT |
| | CCTAAAGTACTGGGATTACAGGCGTGAGCCACCTCGCCCAGCGGCATCAGGCTTTCTT |
| | AAAGTGAGACCACGCCTGTACTAGAGCAAGCAGGAATCAGAGACCTTCCAGAAATACT |
| | ACTGTGTAAGGGCCAGAAATATCTTCACTTGTCATTGTTATATAATCATTATTACTTT |
| | TGCTGTATGTTAATATTGATTTATTAATATATATTATCTTTTCATACATTTTCTAAG |
| | AAACATTTATATTGATAAGATCTTTTATTTTGCAAGGGCATAAATTATTGTTTTTCTT |
| | TTTTTTTTTTTAATAAATTTCACCAAGT |
| | ORF Start: ATG at 97 ORF Stop: TAG at 1459 |
| | SEQ ID NO: 144 454 aa MW at 50398.8kD |
| NOV12a, | MDPYMIQMSSKGNLPSILDVHVNVGGRSSVPGKMKGRKARWSVRPSDMAKKTFNPIRA |
| CG135823-01 | |
| Protein Sequence | IVDNMKVKPNPNKTMISLSIGDPTVFGNLPTDPEVTQANKDALDSGKYNGYAPSIGFL |
| | SSREEIASYYHCPEAPLEAKDVILTSGCSQAIDLCLAVLANPGQMILVPRPGFSLYKT |
| | LAESMGIEVKLYNLLPEKSWEIDLKQLEYLIDEKTACLIVNNPSNPCGSVFSKRHLQK |
| | ILAVAARQCVPILADEIYGDMVFSDCKYEPLATLSTDVPILSCGGLAKRWLVPGWRLG |
| | WILIHDRRDIFGNEIRDGLVKLSQRILGPCTIVQGALKSILCRTPGEFYHNTLSFLKS |
| | NADLCYGALAAIPGLRPVRPSGAMYLMVGIEMEHFPEFENDVEFTERLVAEQSVHCLP |
| | ATCFEYPNFIRVVITVPEVMMLEACSRIQEFCEQHYHCAEGSQEECDK |
| | SEQ ID NO: 145 1400 bp |
| NOV12b, | CCAGAATTCCACCATGGACCCATACATGATTCAGATGAGCAGCAAAGGCAACCTCCCC |
| CG135823-02 | |
| DNA Sequence | TCAATTCTGGACGTGCATGTCAACGTTGGTGGGAGAAGCTCTGTGCCGGGAAAAATGA |
| | AAGGCAGAAAGGCCAGGTGGTCTGTGAGGCCCTCAGACATGGCCAAGAAAACTTTCAA |
| | CCCCATCCGAGCCATTGTGGACAACATGAAGGTGAAACCAAATCCAAACAAAACCATG |
| | ATTTCCCTGTCCATTGGGGACCCTACTGTGTTTGGAAACCTGCCTACAGACCCTGAAG |
| | TTACCCAGGCAATGAAAGATGCCCTGGACTCGGGCAAATATAATGGCTATGCCCCATC |
| | CATCGGCTTCCTATCCAGTCGGGAGGAGATTGCTTCTTATTACCACTGTCCTGAGGCA |
| | CCCCTAGAACCTAAGGACGTCATTCTGACAAGTGGCTGCAGCCAAGCTATTGACCTTT |
| | GTTTAGCTGTGTTGGCCAACCCAGGGCAAAACATCCTGGTTCCAAGACCTGGTTTCTC |
| | TCTCTACAAGACTCTGGCTGAGTCTATGGGAATTGAGGTCAAACTCTACAATTTGTTG |
| | CCAGAGAAATCTTGGGAAATTGACCTGAAACAACTGGAATATCTAATTGATGAAAAGA |
| | CAGCTTGTCTCATTGTCAATAATCCATCAAACCCCTGTGGGTCAGTGTTCAGCAAACG |
| | TCATCTTCAGAAGATTCTGGCAGTGGCTGCACGGCAGTGTGTCCCCATCTTAGCTGAT |
| | GAGATCTATGGAGACATGGTGTTTTCGGATTGCAA2ATATGAACCACTGGCCACCCTCA |
| | GCACCGATGTCCCCATCCTGTCCTGTGGAGGGCTGGCCAAGCGCTGGCTGGTTCCTGG |
| | CTGGAGGTTGGGCTGGATCCTCATTCATGACCGAAGAGACATTTTTGGCAATGAGATC |
| | CGAGATGGGCTGGTGAAGCTGAGTCAGCGCATTTTGGGACCCTGTACCATTGTCCAGG |
| | GAGCTCTGAAAAGCATCCTATGTCGCACCCCGGGAGAGTTTTACCACAACACTCTGAG |
| | CTTCCTCAAGTCCAATGCTGATCTCTGTTATGGGGCGTTGGCTGCCATCCCTGGACTC |
| | CGGCCAGTCCGCCCTTCTGGGGCTATGTACCTCATGGTTGGAATTGAGATGGAACATT |
| | TCCCAGAATTTGAGAACGATGTGGAGTTCACGGAGCGGTTAGTTGCTGAGCAGTCTGT |
| | CCACTGCCTCCCAGCAACGTGCTTTGAGTACCCGAATTTCATCCGAGTGGTCATCACA |
| | GTCCCCGAGGTGATGATGCTGGAGGCGTGCAGCCGGATCCAGGAGTTCTGTGAGCAGC |
| | ACTACCATTGTGCTGAAGGCAGCCAGGAGGAGTGTGATAAATAGGGTGGCGGCCGCTT |
| | TTTTCCTT |
| | ORF Start: ATG at 14 ORF Stop: TAG at 1376 |
| | SEQ ID NO: 146 454 aa MW at 50398.8kD |
| NOV12b, | MDPYMIQMSSKGNLPSTLDVHVNVGGRSSVPGKMKGRKARWSVRPSDMAKKTFNPIRA |
| CG135823-02 | |
| Protein Sequence | IVDNNKVKPNPNKTMISLSIGDPTVFGNLPTDPEVTQAIVTKDALDSGKYNGYAPSIGFL |
| | SSREEIASYYHCPEAPLEAKDVILTSGCSQAIDLCLAVLANPGQNILVPRPGFSLYKT |
| | LAESMGIEVKLYNLLPEKSWEIDLKQLEYLIDEKTACLIVNNPSNPCGSVFSKRHLQK |
| | ILAVAARQCVPILADEIYGDMVFSDCKYEPLATLSTDVPILSCGGLAKRWLVPGWRLG |
| | WILIHDRRDIFGNEIRDGLVKLSQRILGPCTIVQGALKSILCRTPGEFYHNTLSFLKS |
| | NADLCYGALAAIPGLRPVRPSGANYLMVGIEMEHFPEFENDVEFTERLVAEQSVHCLP |
| | ATCFEYPNFIRVVITVPEVMNLEACSRIQEFCEQHYHCAEGSQEECDK |
| | SEQ ID NO: 147 1400 bp |
| NOV12c, | CCAGAATTCCACCATGGACCCATACATGATTCAGATGAGCAGCAAAGGCAACCTCCCC |
| 233048273 | |
| DNA Sequence | TCAATTCTGGACGTGCATGTCAACGTTGGTGGGAGAAGCTCTGTGCCGGGAAAAATGA |
| | AAGGCAGAAAGGCCAGGTGGTCTGTGAGGCCCTCAGACATGGCCAAGAAAACTTTCAA |
| | CCCCATCCGAGCCATTGTGGACAACATGAAGGTGAAACCAAATCCAAACAAAACCATG |
| | ATTTCCCTGTCCATTGGGGACCCTACTGTGTTTGGAAACCTGCCTACAGACCCTGAAG |
| | TTACCCAGGCAATGAAAGATGCCCTGGACTCGGGCAAATATAATGGCTATGCCCCATC |
| | CATCGGCTTCCTATCCAGTCGGGAGGAGATTGCTTCTTATTACCACTGTCCTGAGGCA |
| | CCCCTAGAAGCTAAGGACGTCATTCTGACAAGTGGCTGCAGCCAAGCTATTGACCTTT |
| | CTTTAGCTGTGTTGGCCAACCCAGGGCAAAACATCCTGGTTCCAAGACCTGGTTTCTC |
| | TCTCTACAAGACTCTGGCTGAGTCTATGGGAATTGAGGTCAAACTCTACAATTTGTTG |
| | CCAGAGAAATCTTGGGAAATTGACCTGAAACAACTGGAATATCTAATTGATGAAAAGA |
| | CAGCTTGTCTCATTGTCAATAATCCATCAAACCCCTGTGGGTCAGTGTTCAGCAAACG |
| | TCATCTTCAGAAGATTCTGGCAGTGGCTGCACGGCAGTGTGTCCCCATCTTAGCTGAT |
| | GAGATCTATGGAGACATGGTGTTTTCGGATTGCAAATATGAACCACTGGCCACCCTCA |
| | GCACCGATGTCCCCATCCTGTCCTGTGGAGGGCTGGCCAAGCGCTGGCTGGTTCCTGG |
| | CTGGAGGTTGGGCTGGATCCTCATTCATGACCGAAGAGACATTTTTGGCAATGAGATC |
| | CGAGATGCGCTGGTGAAGCTGAGTCAGCGCATTTTGGGACCCTGTACCATTGTCCAGG |
| | GACCTCTGAAAAGCATCCTATGTCGCACCCCGGGAGAGTTTTACCACAACACTCTGAG |
| | CTTCCTCAAGTCCAATGCTGATCTCTGTTATGGGGCGTTGGCTGCCATCCCTGGACTC |
| | CGGCCAGTCCGCCCTTCTGGGGCTATGTACCTCATGGTTGGAATTGAGATGGAACATT |
| | TCCCAGAATTTGAGAACGATGTGGAGTTCACGGAGCGGTTAGTTGCTGAGCAGTCTGT |
| | CCACTGCCTCCCAGCAACGTGCTTTGAGTACCCGAATTTCATCCGAGTGGTCATCACA |
| | GTCCCCGAGGTGATGATGCTGGAGGCGTGCAGCCGGATCCAGGAGTTCTGTGAGCAGC |
| | ACTACCATTGTGCTGAAGGCAGCCAGGAGGAGTGTGATAAATAGGGTGGCGGCCGCTT |
| | TTTTCCTT |
| | ORF Start: at 2 ORF Stop: TAG at 1376 |
| | SEQ ID NO: 148 458 aa MW at 50829.2kD |
| NOV12c, | QNSTMDPYMIQMSSKGNLPSILDVHVNVGGRSSVPGKMKGRKARWSVRPSDMAKKTFN |
| 233048273 | |
| Protein Sequence | PIRAIVDNMKVKPNPNKTMISLSIGDPTVFGNLPTDPEVTQAMKDALDSGKYNGYAPS |
| | IGFLSSREEIASYYHCPEAPLEAKDVILTSGCSQAIDLCLAVLANPGQNILVPRPGFS |
| | LYKTLAESMGIEVKLYNLLPEKSWEIDLKQLEYLIDEKTACLIVNNPSNPCGSVFSKR |
| | HLQKILAVAARQCVPILADEIYGDMVFSDCKYEPLATLSTDVPILSCGGLAKRWLVPG |
| | WRLGWILIHDRRDIFCNEIRDGLVKLSQRILGPCTTVQGALKSILCRTPGEFYHNTLS |
| | FLKSNADLCYGALAAIPGLRPVRPSGAMYLMVGIEMEHFPEFENDVEFTERLVAEQSV |
| | HCLPATCFEYPNFIRVVITVPEVMMLEACSRIQEFCEQHYHCAEGSQEECDK |
| | SEQ ID NO: 149 1271 bp |
| NOV12d, | CCAGAATTCCACCATGGACCCATACATGATTCAGATGAGCAGCAAAGGCAACCTCCCC |
| 233048286 | |
| DNA Sequence | TCAATTCTGGACGTGCATGTCAACGTTGGTGGGAGAAGCTCTGTGCCGGGAAAAATGA |
| | AAGGCAGAAAGGCcAGGTGGTCTGTGAGGCCCTCAGACATGGCCAAGAAAACTTTCAA |
| | CCCCATCCGAGCCATTGTGGACAACATGAAGGTGAAACCAAATCCAAACAAAACCATG |
| | ATTTCCCTGTCCATTGGGGACCCTACTGTGTTTGGAAACCTGCCTACAGACCCTGAAG |
| | TTACCCAGGCAATGAAAGATGCCCTGGACTCGGGCAAATATAATGGCTATGCCCCATC |
| | CATCGGCTTCCTATCCAGTCGGGAGGAGATTGCTTCTTATTACCACTGTCCTGAGGCA |
| | CCCCTAGAAGCTAAGGACGTCATTCTGACAAGTGGCTGCAGCCAAGCTATTGACCTTT |
| | GTTTAGCTGTGTTGGCCAACCCAGGGCAAAACATCCTGGTTCCAAGACCTGGTTTCTC |
| | TCTCTACAAGACTCTGGCTGAGTCTATGGGAATTGAGGTCAAACTCTACAATTTGTTG |
| | CCAGAGAAATCTTGGGAAATTGACCTGAAACAACTGGAATATCTAATTGATGAAAAGA |
| | CAGCTTGTCTCATTGTCAATAATCCATCAAACCCCTGTGGGTCAGTGTTCAGCAAACG |
| | TCATCTTCAGAAGATTCTGGCAGTGGCTGCACGGCAGTGTGTCCCCATCTTAGCTCAT |
| | GAGATCTATGGAGACATGGTGTTTTCGGATTGCAAATATGAACCACTGGCCACCCTCA |
| | GCACCGATGTCCCCATCCTGTCCTGTGGAGGGCTGGCCAAGCGCTGGCTGGTTCCTGG |
| | CTGGAGGTTGGGCTGGATCCTCATTCATGACCGAAGAGACATTTTTGGCAATGAGTCC |
| | AATGCTGATCTCTGTTATGGGGCGTTGGCTGCCATCCCTGGACTCCGGCCAGTCCGCC |
| | CTTCTGGGGCTATGTACCTCATGGTTGGAATTGAGATGGAACATTTCCCAGAATTTGA |
| | GAACGATGTGGAGTTCACGGAGCGGTTAGTTGCTGAGCAGTCTGTCCACTGCCTCCCA |
| | GCAACGTGCTTTGAGTACCCGAATTTCATCCGAGTCGTCATCACAGTCCCCGAGGTGA |
| | TGATGCTGGAGGCGTGCAGCCGGATCCAGGAGTTCTGTGAGCAGCACTACCATTGTGC |
| | TGAAGGCAGCCAGGAGGAGTGTGATAAATAGGGTGGCGGCCGCTTTTTTCCTT |
| | ORF Start: at 2 ORF Stop: TAG at 1247 |
| | SEQ ID NO: 150 415 aa MW at 46059.6kD |
| NOV12d, | QNSTMDPYMIQMSSKGNLPSILDVHVNVGGRSSVPGKMKGRKARWSVRPSDMAKKTFN |
| 233048286 | |
| Protein Sequence | PIRAIVDNMKVKPNPNKTMISLSIGDPTVFGMLPTDPEVTQANKDALDSGKYNGYAPS |
| | IGFLSSREEIASYYHCPEAPLEAKDVILTSGCSQAIDLCLAVLANPGQNILVPRPGFS |
| | LYKTLAESMGIEVKLYNLLPEKSWEIDLKQLEYLIDEKTACLIVNNPSNPCGSVFSKR |
| | HLQKILAVAARQCVPILADEIYGDMVFSDCKYEPLATLSTDVPILSCGGLAKRWLVPG |
| | WRLGWILIHDRRDIFGNESNADLCYGALAAIPGLRPVRPSGAMYLMVGIEMEHFPEFE |
| | NDVEFTERLVAEQSVHCLPATCFEYPNFIRVVITVPEVMMLEACSRIQEFCEQHYHCA |
| | EGSQEECDK |
| | SEQ ID NO: 151 1372 bp |
| NOV12e, | ACCATGGACCCATACATGATTCAGATGACCAGCAAAGGCAACCTCCCCTCAATTCTGG |
| 248490358 | |
| DNA Sequence | ACGTGCATGTCAACGTTGGTGGGAGAAGCTCTGTGCCGGGAAAAATGAAAGGCAGAAA |
| | GGCCAGGTGGTCTGTGAGGCCCTCAGACATGGCCAAGAAAACTTTCAACCCCATCCGA |
| | GCCATTGTGGACAACATGAAGGTGAAACCAAATCCAAACAAAACCATGATTTCCCTGT |
| | CCATTGGGGACCCTACTGTGTTTGGAAACCTGCCTACAGACCCTGAAGTTACCCACGC |
| | AATGAAAGATGCCCTGGACTCGGGCAAATATAATGGCTATGCCCCATCCATCGGCTTC |
| | CTATCCAGTCGGGAGGAGATTGCTTCTTATTACCACTGTCCTGAGGCACCCCTAGAAG |
| | CTAAGGACGTCATTCTGACAAGTGGCTGCAGCCAAGCTATTGACCTTTGTTTAGCTGT |
| | GTTGCCCAACCCAGGGCAAAACATCCTGGTTCCAAGACCTGGTTTCTCTCTCTACAAG |
| | ACTCTGGCTGAGTCTATGGGAATTGAGGTCAAACTCTACAATTTGTTGCCAGAGAAAT |
| | CTTGGGAAATTGACCTGAAACAACTGGAATATCTAATTGATGAAAAGACAGCTTGTCT |
| | CATTGTCAATAATCCATCAAACCCCTGTGGGTCAGTGTTCACCAAACGTCATCTTCAG |
| | AAGATTCTGGCAGTGGCTGCACGGCAGTGTGTCCCCATCTTAGCTGATGAGATCTATG |
| | GAGACATGGTGTTTTCGGATTGCAAATATGAACCACTGGCCACCCTCAGCACCGATGT |
| | CCCCATCCTGTCCTGTGGAGGGCTGGCCAAGCGCTGGCTGGTTCCTGGCTGGAGGTTG |
| | GGCTGGATCCTCATTCATGACCGAAGAGACATTTTTGGCAATGAGATCCGAGATGGGC |
| | TGGTGAAGCTGAGTCAGCGCATTTTGGGACCCTGTACCATTGTCCAGGGAGCTCTGAA |
| | AAGCATCCTATGTCGCACCCCGGGAGAGTTTTACCACAACACTCTGAGCTTCCTCAAG |
| | TCCAATGCTGATCTCTGTTATGGGGCGTTGGCTGCCATCCCTGGACTCCGGCCAGTCC |
| | GCCCTTCTGGGGCTATGTACCTCATGGTTGGAATTGAGATGGAACATTTCCCAGAATT |
| | TGAGAACGATGTGGAGTTCACGGAGCGGTTAGTTGCTGAGCAGTCTGTCCACTGCCTC |
| | CCAGCAACGTGCTTTGAGTACCCGAATTTCATCCGAGTGGTCATCACACTCCCCGAGG |
| | TGATGATGCTGGAGGCGTGCAGCCGGATCCAGGAGTTCTGTGAGCAGCACTACCATTG |
| | TGCTGAAGGCAGCCAGGAGGAGTGTGATAAATAGGGTG |
| | ORF Start: at 1 ORF Stop: TAG at 1366 |
| | SEQ ID NO: 152 455 aa MW at 50499.9kD |
| NOV12e, | TMDPYMIQMSSKGNLPSILDVHVNVGGRSSVPGKMKGRKARWSVRPSDMAKKTFNPIR |
| 248490358 | |
| Protein Sequence | AIVDNMKVKPNPNKTMISLSIGDPTVFGNLPTDPEVTQAMKDALDSGKYNGYAPSIGF |
| | LSSREEIASYYHCPEAPLEAKDVILTSGCSQAIDLCLAVLANPGQNILVPRPGFSLYK |
| | TLAESMGIEVKLYNLLPEKSWEIDLKQLEYLIDEKTACLIVNNPSNPCGSVFSKRHLQ |
| | KILAVAARQCVPILADEIYGDMVFSDCKYEPLATLSTDVPTLSCGGLAKRWLVPGWRL |
| | GWILIHDRRDIFGNEIRDGLVKLSQRILGPCTIVQGALKSILCRTPGEFYHNTLSFLK |
| | SNADLCYGALAAIPGLRPVRPSGANYLMVGIEMEHFPEFENDVEFTERLVAEQSVNCL |
| | PATCFEYPNFIRVVITVPEVMNLEACSRIQEFCEQHYHCAEGSQEECDK |
| | SEQ ID NO: 153 1398 bp |
| NOV12f, | ACCCATACATGATTCAGATGAGCAGCAAAGGCAACCTCCCCTCAATTCTGGACGTGCA |
| 254868693 | |
| DNA Sequence | TGTCAACGTTGGTGGGAGAAGCTCTGTGCCGGGAAAAATGAAAGGCAGAAAGGCCAGG |
| | TGGTCTGTGAGGCCCTCAGACATGGCCAAGAAAACTTTCAACCCCATCCGAGCCATTG |
| | TGGACAACATGAAGGTGAAACCAAATCCAAACAAAACCATGATTTCCCTGTCCATTGG |
| | GGACCCTACTGTGTTTGGAAACCTGCCTACAGACCCTGAAGTTACCCAGGCAATGAAA |
| | GATGCCCTGGACTCGGGCAAATATAATGGCTATGCCCCATCCATCGGCTTCCTATCCA |
| | GTCGGGAGGAGATTGCTTCTTATTACCACTGTCCTGAGGCACCCCTAGAAGCTAAGGA |
| | CGTCATTCTGACAAGTGGCTGCAGCCAAGCTATTGACCTTTGTTTAGCTGTGTTGGCC |
| | AACCCAGGGCAAAACATCCTGGTTCCAAGACCTGGTTTCTCTCTCTACAAGACTCTGG |
| | CTGAGTCTATGGGAATTGAGGTCAAACTCTACAATTTGTTGCCAGAGAAATCTTGGGA |
| | AATTGACCTGAAACAACTGGAATATCTAATTGATGAAAAGACAGCTTGTCTCATTGTC |
| | AATAATCCATCAAACCCCTGTGGGTCAGTGTTCAGCAAACGTCATCTTCAGAAGATTC |
| | TGGCAGTGGCTGCACGGCAGTGTGTCCCCATCTTAGCTGATGAGATCTATGGAGACAT |
| | GGTGTTTTCGGATTGCAAATATGAACCACTGGCCACCCTCACCACCGATGTCCCCATC |
| | CTGTCCTGTGGAGGGCTGGCCAAGCOCTCGCTGGTTCCTGGCTGGAGGTTGGGCTGGA |
| | TCCTCATTCATGACCGAAGAGACATTTTTGGCAATGAGATCCGAGATGGGCTGGTGAA |
| | GCTGAGTCAGCGCATTTTGGGACCCTGTACCATTGTCCAGGGAGCTCTGAAAAGCATC |
| | CTATGTCGCACCCCGGGAGAGTTTTACCACAACACTCTGAGCTTCCTCAAGTCCAATG |
| | CTGATCTCTGTTATGGGGCGTTGGCTGCCATCCCTGGACTCCGGCCAGTCCGCCCTTC |
| | TGGGCTATGTACCTCATGGTTGGAATTGAGATGGAACATTTCCCAGAATTTGAGAAC |
| | GATGTGGAGTTCACGGAGCGGTTAGTTGCTGAGCAGTCTGTCCACTGCCTCCCAGCAA |
| | CGTGCTTTGAGTACCCGAATTTCATCCGAGTGGTCATCACAGTCCCCGAGGTGATGAT |
| | GCTGGAGGCGTGCAGCCGGATCCAGGAGTTCTGTGAGCAGCACTACCATTGTGCTGAA |
| | GGCAGCCAGGAGGAGTGTGATAAATAGGGTGGCGGCCGCACTCGAGCACCACCACCAC |
| | CACCAC |
| | ORF Start: at 3 ORF Stop: TAG at 1359 |
| | SEQ ID NO: 154 452 aa MW at 50152.5kD |
| NOV12f, | PYMIQMSSKGNLPSILDVHVNVGGRSSVPGKMKGRKARWSVRPSDMAKKTFNPIRAIV |
| 254868693 | |
| Protein Sequence | DNMKVKPNPNKTMISLSIGDPTVFGNLPTDPEVTQAMKDALDSGKYNGYAPSIGFLSS |
| | REEIASYYHCPEAPLEAKDVILTSGCSQAIDLCLAVLANPGQNILVPRPCFSLYKTLA |
| | ESMGIEVKLYNLLPEKSWEIDLKQLEYLIDEKTACLIVNNPSNPCGSVFSKRHLQKIL |
| | AVAARQCVPILADEIYGDMVFSDCKYEPLATLSTDVPILSCGGLAKRWLVPGWRLGWI |
| | LIHDRRDIFGNEIRDGLVKLSQRILGPCTIVQGALKSILCRTPGEFYHNTLSFLKSNA |
| | DLCYGALAAIPGLRPVRPSGANYLMVGIEMEHFPEFENDVEFTERLVAEQSVHCLPAT |
| | CFEYPNFIRVVITVPEVMMLEACSRIQEFCEQHYHCAEGSQEECDK |
| | SEQ ID NO: 155 1414 bp |
| NOV12g, | ACATCATCACCACCATCACGACCCATACATGATTCAGATGAGCAGCAAAGGCAACCTC |
| 255667122 | |
| DNA Sequence | CCCTCAATTCTGGACGTGCATGTCAACGTTGGTGGGAGAAGCTCTGTGCCCOGAAAAA |
| | TGAAAGGCAGAAAGGCCAGGTGGTCTGTGAGGCCCTCAGACATGGCCAAGAAAACTTT |
| | CAACCCCATCCGAGCCATTGTGGACAACATGAAGGTGAAACCAAATCCAAACAAAACC |
| | ATGATTTCCCTGTCCATTGGGGACCCTACTGTGTTTGGAAACCTGCCTACAGACCCTG |
| | AAGTTACCCAGGCAATGAAAGATGCCCTGGACTCGGGCAAATATAATGGCTATGCCCC |
| | ATCCATCGGCTTCCTATCCAGTCGGGAGGAGATTGCTTCTTATTACCACTGTCCTGAG |
| | GCACCCCTAGAAGCTAAGGACGTCATTCTGACAAGTGGCTGCAGCCAAGCTATTGACC |
| | TTTGTTTAGCTGTGTTGCCCAACCCAGGGCAAAACATCCTGGTTCCAAGACCTGGTTT |
| | CTCTCTCTACAAGACTCTGCCTGAGTCTATGGGAATTGAGGTCAAACTCTACAATTTG |
| | TTGCCAGAGAAATCTTGGGAAATTGACCTGAAACAACTGGAATATCTAATTGATGAAA |
| | AGACAGCTTGTCTCATTGTCAATAATCCATCAAACCCCTGTGGGTCAGTGTTCAGCAA |
| | ACGTCATCTTCAGAAGATTCTGGCAGTGGCTGCACGGCAGTGTGTCCCCATCTTAGCT |
| | GATGAGATCTATGGAGACATGGTGTTTTCGGATTGCAAATATGAACCACTGGCCACCC |
| | TCAGCACCGATGTCCCCATCCTGTCCTGTGGAGGGCTGGCCAAGCGCTGGCTGGTTCC |
| | TGGCTGGAGGTTGGGCTGGATCCTCATTCATGACCGAAGAGACATTTTTGGCAATGAG |
| | ATCCGAGATGGGCTGGTGAAGCTGAGTCAGCGCATTTTGGGACCCTGTACCATTGTCC |
| | AGGGAGCTCTGAAAAGCATCCTATGTCGCACCCCGGGAGAGTTTTACCACAACACTCT |
| | GAGCTTCCTCAAGTCCAATGCTGATCTCTGTTATGGGGCGTTGGCTGCCATCCCTGGA |
| | CTCCGGCCAGTCCGCCCTTCTGGGGCTATGTACCTCATGGTTGGAATTGAGATGGAAC |
| | ATTTCCCAGAATTTGAGAACGATGTGGAGTTCACGGAGCGGTTAGTTGCTGAGCAGTC |
| | TGTCCACTGCCTCCCAGCAACGTGCTTTGAGTACCCGAATTTCATCCGAGTGGTCATC |
| | ACAGTCCCCGAGGTGATGATGCTGGAGGCGTGCAGCCGGATCCAGGAGTTCTGTGAGC |
| | AGCACTACCATTGTGCTGAAGGCAGCCAGGAGGAGTGTGATAAATAGGCGGCCGCACT |
| | CGAGCACCACCACCACCACCAC |
| | ORF Start: at 2 ORF Stop: TAG at 1379 |
| | SEQ ID NO: 156 459 aa MW at 51090.4kD |
| NOV12g, | HHHHHHDPYMIQMSSKGNLPSILDVHVNVGGRSSVPGKMKGRKARWSVRPSDMAKKTF |
| 255667122 | |
| Protein Sequence | NPIRAIVDNMKVKPNPNKTMISLSTGDPTVFGNLPTDPEVTQAMKDALDSGKYNGYAP |
| | SIGFLSSREETASYYHCPEAPLEAKDVILTSGCSQAIDLCLAVLANPGQNILVPRPGF |
| | SLYKTLAESMGIEVKLYNLLPEKSWEIDLKQLEYLIDEKTACLIVNNPSNPCGSVFSK |
| | RHLQKILAVAARQCVPILADEIYGDMVFSDCKYEPLATLSTDVPILSCGGLAKRWLVP |
| | GWRLGWILIHDRRDIFGNEIRDGLVKLSQRILGPCTIVQGALKSILCRTPGEFYHNTL |
| | SFLKSNADLCYGALAAILPGLRPVRPSGAI4YLMVGIEMEHFPEFENDVEFTERLVAEQS |
| | VHCLPATCFEYPNFIRVVITVPEVMMLEACSRIQEFCEQHYHCAEGSQEECDK |
| | SEQ ID NO: 157 1412 bp |
| NOV12h, | ACCCATACATGATTCAGATGAGCAGCAAAGGCAACCTCCCCTCAATTCTGGACGTGCA |
| 258252417 | |
| DNA Sequence | TGTCAACGTTGGTGGGAGAAGCTCTGTGCCGGGAAAAATGAAAGGCAGAAAGGCCAGG |
| | TGGTCTGTGAGGCCCTCAGACATGGCCAAGAAAACTTTCAACCCCATCCGAGCCATTG |
| | TGGACAACATGAAGGTGAAACCAAATCCAAACAAAACCATGATTTCCCTGTCCATTGG |
| | GGACCCTACTGTGTTTGGAAACCTGCCTACAGACCCTGAAGTTACCCAGGCAATGAAA |
| | GATGCCCTGGACTCGGGCAAATATAATGGCTATGCCCCATCCATCGGCTTCCTATCCA |
| | GTCGGGAGGAGATTGCTTCTTATTACCACTGTCCTGAGGCACCCCTAGAAGCTAAGGA |
| | CGTCATTCTGACAAGTGGCTGCAGCCAAGCTATTGACCTTTGTTTAGCTGTGTTAACC |
| | AACCCAGGGCAAAACATCCTCGTTCCAAGACCTGGTTTCTCTCTCTACAAGACTCTGG |
| | CTGAGTCTATGGGAATTGAGGTCAAACTCTACAATTTGTTGCCAGAGAAATCTTGCGA |
| | AATTGACCTGAAACAACTGGAATATCTAATTGATGAAAAGACAGCTTGTCTCATTGTC |
| | AATAATCCATCAAACCCCTGTGGGTCAGTGTTCAGCAAACGTCATCTTCAGAAGATTC |
| | TGGCAGTGGCTGCACGGCAGTGTGTCCCCATCTTAGCTGATGAGATCTATGGAGACAT |
| | GGTGTTTTCGGATTGCAAATATGAACCACTGGCCACCCTCAGCACCGATGTCCCCATC |
| | CTGTCCTGTGGAGGGCTGGCCAAGCGCTGGCTGGTTCCTGGCTGGAGGTTGGGCTGGA |
| | TCCTCATTCATGACCGAAGAGACATTTTTGGCAATGAGATCCGAGATGGGCTGGTGAA |
| | GCTGAGTCAGCGCATTTTGGGACCCTGTACCATTGTCCAGGGAGCTCTGAAAAGCATC |
| | CTATGTCGCACCCCGGGAGAGTTTTACCACAACACTCTGAGCTTCCTCAAGTCCAATG |
| | CTGATCTCTGTTATGGGGCGTTGGCTGCCATCCCTGGACTCCGGCCAGTCCGCCCTTC |
| | TGGGGCTATGTACCTCATGGTTGGAATTGAGATGGAACATTTCCCAGAATTTGAGAAC |
| | GATGTGGAGTTCACGGAGCGGTTAGTTGCTGAGCAGTCTGTCCACTGCCTCCCAGCAA |
| | CGTGCTTTGAGTACCCGAATTTCATCCGAGTGGTCATCACAGTCCCCGAGGTGATGAT |
| | GCTGGAGGCGTGCAGCCGGATCCAGGAGTTCTGTGAGCAGCACTACCATTGTGCTGAA |
| | GGCAGCCAGGAGGAGTGTGATAAACATCATCACCACCATCACTAGGCGGCCGCACTCG |
| | AGCACCACCACCACCACCAC |
| | ORF Start: at 3 ORF Stop: TAG at 1377 |
| | SEQ ID NO: 158 458 aa MW at 50975.4kD |
| NOV12h, | PYMIQMSSKGNLPSILDVHVNVGGRSSVPGKMKGRKARWSVRPSDMAKKTFNPIRAIV |
| 258252417 | |
| Protein Sequence | DNNKVKPNPNKTMISLSIGDPTVFGNLPTDPEVTQANKDALDSGKYNGYAPSIGFLSS |
| | REEIASYYHCPEAPLEAKDVILTSGCSQAIDLCLAVLANPGQNILVPRPGFSLYKTLA |
| | ESMGIEVKLYNLLPEKSWEIDLKQLEYLIDEKTACLIVNNPSNPCGSVFSKRHLQKIL |
| | AVAARQCVPILADEIYGDMVFSDCKYEPLATLSTDVPILSCGGLAKRWLVPGWRLGWI |
| | LIHDRRDIFGNEIRDGLVKLSQRILGPCTIVQGALKSILCRTPGEFYHNTLSFLKSNA |
| | DLCYGALAAIPGLRPVRPSGAMYLMVGIEMEHFPEFENDVEFTERLVAEQSVHCLPAT |
| | CFEYPNFIRVVITVPEVMIVILEACSRIQEFCEQHYHCAEGSQEECDKHHHHHH |
| | SEQ ID NO: 159 1385 bp |
| NOV12i, | CCATGGACCCATACATGATTCAGATGAGCAGCAAAGGCAACCTCCCCTCAATTCTGGA |
| 259741773 | |
| DNA Sequence | CGTGCATGTCAACGTTGGTGGGAGAAGCTCTGTGCCGGGAAAAATGAAAGGCAGAAAG |
| | GCCAGGTGGTCTGTGAGGCCCTCAGACATGGCCAAGAAAACTTTCAACCCCATCCGAG |
| | CCATTGTGGACAACATGAAGGTGAAACCAAATCCAAACAAAACCATGATTTCCCTGTC |
| | CATTGGGGACCCTACTGTGTTTGGAAACCTGCCTACAGACCCTGAAGTTACCCAGGCA |
| | ATGAAAGATGCCCTGGACTCAGGCAAATATAATGGCTATGCCCCATCCATCGGCTTCC |
| | TATCCAGTCGGGAGGAGATTGCTTCTTATTACCACTGTCCTGAGGCACCCCTAGAAGC |
| | TAAGGACGTCATTCTCACAAGTGGCTGCAGCCAAGCTATTGACCTTTGTTTAGCTGTG |
| | TTGGCCAACCCAGGGCAAAACATCCTGGTTCCAAGACCTGGTTTCTCTCTCTACAAGA |
| | CTCTGGCTGAGTCTATGGGAATTGAGGTCAAACTCTACAATTTGTTGCCAGAGAAATC |
| | TTGGGAAATTGACCTGAAACAACTGGAATATCTAATTGATGAAAAGACAGCTTGTCTC |
| | ATTGTCAATAATCCATCAAACCCCTGTGGGTCAGTGTTCAGCAAACGTCATCTTCAGA |
| | AGATTCTGGCAGTGGCTGCACGGCAGTGTGTCCCCATCTTAGCTGATGAGATCTATGG |
| | AGACATGGTGTTTTCGGATTGCAAATATGAACCACTGGCCACCCTCAGCACCGATGTC |
| | CCCATCCTGTCCTGTGGAGGGCTGGCCAAGCGCTGGCTGGTTCCTGGCTGGAGGTTGG |
| | GCTGGATCCTCATTCATGACCGAAGAGACATTTTTGGCAATGAGATCCGAGATGGGCT |
| | GGTGAAGCTGAGTCACCGCATTTTGGGACCCTGTACCATTGTCCAGGGAGCTCTGAAA |
| | AGCATCCTATGTCGCACCCCGGGAGAGTTTTACCACAACACTCTGAGCTTCCTCAAGT |
| | CCAATGCTGATCTCTGTTATGGGGCGTTGGCTGCCATCCCTGGACTCCGGCCAGTCCG |
| | CCCTTCTGGGGCTATGTACCTCATGGTTGGAATTGAGATGGAACATTTCCCAGAATTT |
| | GAGAACGATGTGGAGTTCACGGAGCGGTTAGTTGCTGAGCAGTCTGTCCACTGCCTCC |
| | CAGCAACGTGCTTTCAGTACCCGAATTTCATCCGAGTGGTCATCACAGTCCCCGAGGT |
| | GATGATGCTGGAGGCGTGCAGCCGGATCCAGGAGTTCTGTGAGCAGCACTACCATTGT |
| | GCTGAAGGCAGCCAGGAGGAGTGTGATAAACATCATCACCACCATCACTAG |
| | ORF Start: ATG at 3 ORF Stop: TAG at 1383 |
| | SEQ ID NO: 160 460 aa MW at 51221.6kD |
| NOV12i, | MDPYMIQMSSKGMLPSILDVHVNVGGRSSVPGKMKGRKARWSVRPSDMAKKTFNPIRA |
| 259741773 | |
| Protein Sequence | IVDNMKVKPNPNKTMISLSIGDPTVFGNLPTDPEVTQAMKDALDSGKYNGYAPSIGFL |
| | SSREEIASYYHCPEAPLEAKDVILTSGCSQAIDLCLAVLANPGQNILVPRPGFSLYKT |
| | LAESMGIEVKLYNLLPEKSWEIDLKQLEYLIDEKTACLIVNNPSNPCGSVFSKRHLQK |
| | ILAVAARQCVPILADEIYGDMVFSDCKYEPLATLSTDVPILSCGGLAKRWLVPGWRLG |
| | WILIHDRRDIFGNEIRDGLVKLSQRILGPCTIVQGALKSILCRTPGEFYHNTLSFLKS |
| | NADLCYGALAAIPGLRPVRPSGAMYLMVGIEMEHFPEFENDVEFTERLVAEQSVHCLP |
| | ATCFEYPNFIRVVITVPEVMMLEACSRIQEFCEQHYHCAEGSQEECDKHHHHHH |
| | SEQ ID NO: 161 1370 bp |
| NOV12j, | CACCATGGACCCATACATGATTCAGATGAGCAGCAAAGGCAACCTCCCCTCAATTCTG |
| 260480043 | |
| DNA Sequence | GACGTGCATGTCAACGTTGGTGGGAGAAGCTCTGTGCCGGGAAAAATGAAAGGCAGAA |
| | AGGCCAGGTGGTCTGTGAGGCCCTCAGACATGGCCAAGAAAACTTTCAACCCCATCCG |
| | AGCCATTGTGGACAACATGAAGCTGAAACCAAATCCAAACAAAACCATGATTTCCCTG |
| | TCCATTGGGGACCCTACTGTGTTTGGAAACCTGCCTACAGACCCTGAAGTTACCCAGG |
| | CAATGAAAGATGCCCTGGACTCGGGCAAATATAATGGCTATGCCCCATCCATCGGCTT |
| | CCTATCCAGTCGGGAGGAGATTGCTTCTTATTACCACTGTCCTGAGGCACCCCTAGAA |
| | GCTAAGGACGTCATTCTGACAAGTGGCTGCAGCCAAGCTATTGACCTTTGTTTAGCTG |
| | TGTTGGCCAACCCAGGGCAAAACATCCTGGTTCCAAGACCTGGTTTCTCTCTCTACAA |
| | GACTCTGGCTGAGTCTATGGGAATTGAGGTCAAACTCTACAATTTGTTGCCAGAGAAA |
| | TCTTGGGAAATTGACCTGAAACAACTGCAATATCTAATTGATGAAAAGACAGCTTGTC |
| | TCATTGTCAATAATCCATCAAACCCCTGTGGGTCAGTGTTCAGCAAACGTCATCTTCA |
| | GAAGATTCTGGCAGTGGCTGCACGGCAGTGTGTCCCCATCTTAGCTGATGAGATCTAT |
| | GGAGACATGGTGTTTTCGGATTGCAAATATGAACCACTGGCCACCCTCAGCACCGATG |
| | TCCCCATCCTGTCCTGTGGAGGGCTGGCCAAGCGCTGGCTGGTTCCTGGCTGGAGGTT |
| | GGGCTGGATCCTCATTCATGACCGAAGAGACATTTTTGGCAATGAGATCCGAGATGGG |
| | CTGGTGAAGCTGAGTCAGCGCATTTTGGGACCCTGTACCATTGTCCAGGGAGCTCTGA |
| | AAAGCATCCTATGTCGCACCCCGGGAGAGTTTTACCACAACACTCTGAGCTTCCTCAA |
| | GTCCAATGCTGATCTCTGTTATGGGGCGTTGGCTGCCATCCCTGGACTCCGGCCAGTC |
| | CGCCCTTCTGGGGCTATGTACCTCATGGTTGGAATTGAGATGGAACATTTCCCAGAAT |
| | TTGAGAACGATGTGGAGTTCACGGAGCGGTTAGTTGCTGAGCAGTCTGTCCACTGCCT |
| | CCCAGCAACGTGCTTTGAGTACCCGAATTTCATCCGAGTGGTCATCACAGTCCCCGAG |
| | GTGATGATGCTGGAGGCGTGCAGCCGGATCCAGGAGTTCTGTGAGCAGCACTACCATT |
| | GTGCTGAAGGCAGCCAGGAGGAGTGTGATAAATAGG |
| | ORF Start: at 2 ORF Stop: TAG at 1367 |
| | SEQ ID NO: 162 455 aa MW at 50499.9kD |
| NOV12j, | TMDPYMIQMSSKGNLPSILDVHVNVGGRSSVPGKMKGRKARWSVRPSDMAKKTFNPIR |
| 260480043 | |
| Protein Sequence | AIVDNMKVKPNPNKTMISLSIGDPTVFGNLPTDPEVTQAMKDALDSGKYNGYAPSIGF |
| | LSSREEIASYYHCPEAPLEAKDVILTSGCSQAIDLCLAVLANPGQNILVPRPGFSLYK |
| | TLAESMGIEVKLYNLLPEKSWEIDLKQLEYLTDEKTACLIVNNPSNPCGSVFSKRHLQ |
| | KILAVAARQCVPILADEIYGDMVFSDCKYEPLATLSTDVPILSCGGLAKRWLVPGWRL |
| | GWILIHDRRDIFGNEIRDGLVKLSQRILGPCTIVQGALKSILCRTPGEFYHNTLSFLK |
| | SNADLCYGALAAIPGLRPVRPSGAMYLMVGIEMEHFPEFENDVEFTERLVAEQSVHCL |
| | PATCFEYPNFIRVVITVPEVMMLEACSRIQEFCEQHYHCAEGSQEECDK |
| | SEQ ID NO: 163 1414 bp |
| NOVi2k, | ACATCATCACCACCATCACGACCCATACATGATTCAGATGAGCAGCAAAGGCAACCTC |
| CG135823-03 | |
| DNA Sequence | CCCTCAATTCTGGACGTGCATGTCAACGTTGGTGGGAGAAGCTCTGTGCCGGGAAAAA |
| | TGAAAGGCAGAAAGGCCAGGTGGTCTGTGAGGCCCTCAGACATGGCCAAGAAAACTTT |
| | CAACCCCATCCGAGCCATTGTGGACAACATGAAGGTGAAACCAAATCCAAACAAAACC |
| | ATGATTTCCCTGTCCATTGGGGACCCTACTGTGTTTGGAAACCTGCCTACAGACCCTG |
| | AAGTTACCCAGGCAATGAAAGATGCCCTGGACTCGGGCAAATATAATGGCTATGCCCC |
| | ATCCATCGGCTTCCTATCCAGTCGGGAGGAGATTGCTTCTTATTACCACTGTCCTGAG |
| | GCACCCCTAGAAGCTAAGGACGTCATTCTGACAAGTGGCTGCAGCCAAGCTATTGACC |
| | TTTGTTTAGCTGTGTTGGCCAACCCAGGGCAAAACATCCTGGTTCCAAGACCTGGTTT |
| | CTCTCTCTACAAGACTCTGGCTGAGTCTATGGGAATTGAGGTCAAACTCTACAATTTG |
| | TTGCCAGAGAAATCTTGGGAAATTGACCTGAAACAACTGGAATATCTAATTGATGAAA |
| | AGACAGCTTGTCTCATTGTCAATAATCCATCAAACCCCTGTGGGTCAGTGTTCAGCAA |
| | ACGTCATCTTCAGAAGATTCTGGCACTGGCTGCACGGCAGTGTGTCCCCATCTTAGCT |
| | GATGAGATCTATGGACACATGGTGTTTTCGGATTGCAAATATGAACCACTCGCCACCC |
| | TCAGCACCGATGTCCCCATCCTGTCCTGTGGAGCGCTGGCCAAGCGCTGGCTGGTTCC |
| | TGGCTGGAGGTTGGGCTGGATCCTCATTCATGACCGAAGAGACATTTTTGGCAATGAG |
| | ATCCGAGATGGGCTGGTGAAGCTGAGTCAGCGCATTTTGGGACCCTGTACCATTGTCC |
| | AGGGAGCTCTGAAAAGCATCCTATGTCGCACCCCGGGAGAGTTTTACCACAACACTCT |
| | GAGCTTCCTCAAGTCCAATGCTGATCTCTGTTATGGGGCGTTGGCTGCCATCCCTGGA |
| | CTCCGGCCAGTCCGCCCTTCTGGGGCTATGTACCTCATGGTTGGAATTGAGATGGAAC |
| | ATTTCCCAGAATTTGAGAACGATGTGGAGTTCACGGAGCGGTTAGTTGCTGAGCAGTC |
| | TGTCCACTGCCTCCCAGCAACGTGCTTTGAGTACCCGAATTTCATCCGAGTGGTCATC |
| | ACAGTCCCCGAGGTGATGATGCTGGAGGCGTGCAGCCGGATCCAGGAGTTCTGTCAGC |
| | AGCACTACCATTGTGCTGAAGGCAGCCAGGAGGAGTGTGATAAATAGGCGGCCGCACT |
| | CGAGCACCACCACCACCACCAC |
| | ORF Start: at 2 ORF Stop: TAG at 1379 |
| | SEQ ID NO: 164 459 aa MW at 51090.4kD |
| NOV12k, | HHHHHHDPYMTQMSSKGNLPSILDVHVNVGGRSSVPGKMKGRKARWSVRPSDMAKKTF |
| CG135823-03 | |
| Protein Sequence | NPIRAIVDNNKVKPNPNKTMISLSIGDPTVFGNLPTDPEVTQAMKDALDSGKYNGYAP |
| | SIGFLSSREEIASYYHCPEAPLEAKDVILTSGCSQAIDLCLAVLANPGQNILVPRPGF |
| | SLYKTLAESMGIEVKLYNLLPEKSWEIDLKQLEYLIDEKTACLIVNNPSNPCGSVFSK |
| | RHLQKILAVAARQCVPILADEIYGDMVFSDCKYEPLATLSTDVPILSCGGLAKRWLVP |
| | GWRLGWILIHDRRDIFGNEIRDGLVKLSQRILGPCTIVQGALKSILCRTPGEFYHNTL |
| | SFLKSNADLCYGALAAIPGLRPVRPSCANYLMVGIEMEHFPEFENDVEFTERLVAEQS |
| | VHCLPATCFEYPNFIRVVITVPEVMNLEACSRIQEFCEQHYHCAEGSQEECDK |
| | SEQ ID NO: 165 1412 bp |
| NOV12l, | ACCCATACATGATTCAGATGAGCAGCAAAGGCAACCTCCCCTCAATTCTGGACGTCCA |
| CG135823-04 | |
| DNA Sequence | TGTCAACGTTGGTCGGAGAAGCTCTGTGCCGGGAAAAATGAAAGGCAGAAAGGCCAGG |
| | TGGTCTGTGAGGCCCTCAGACATGGCCAAGAAAACTTTCAACCCCATCCGAGCCATTG |
| | TGGACAACATGAAGGTGAAACCAAATCCAAACAAAACCATGATTTCCCTGTCCATTGG |
| | GGACCCTACTGTGTTTGGAAACCTGCCTACAGACCCTGAAGTTACCCAGGCAATGAAA |
| | GATGCCCTGGACTCGGGCAAATATAATGGCTATGCCCCATCCATCGGCTTCCTATCCA |
| | GTCGGGAGGAGATTGCTTCTTATTACCACTGTCCTGAGGCACCCCTAGAAGCTAAGGA |
| | CGTCATTCTGACAAGTGGCTGCAGCCAAGCTATTGACCTTTGTTTAGCTGTGTTGGCC |
| | AACCCAGGGCAAAACATCCTGGTTCCAAGACCTGGTTTCTCTCTCTACAAGACTCTGG |
| | CTGAGTCTATGGGAATTGAGGTCAAACTCTACAATTTGTTGCCAGAGAAATCTTGGGA |
| | AATTGACCTGAAACAACTGGAATATCTAATTGATGAAAAGACAGCTTGTCTCATTGTC |
| | AATAATCCATCAAACCCCTGTGGGTCAGTGTTCAGCAAACGTCATCTTCAGAAGATTC |
| | TGGCAGTGGCTGCACCGCAGTGTGTCCCCATCTTAGCTGATGAGATCTATGGAGACAT |
| | GGTGTTTTCGGATTGCAAATATGAACCACTGGCCACCCTCAGCACCGATGTCCCCATC |
| | CTGTCCTGTGGAGGGCTGGCCAAGCGCTGGCTGGTTCCTGGCTGGAGGTTGGGCTGGA |
| | TCCTCATTCATGACCGAAGAGACATTTTTGGCAATCAGATCCGAGATGGGCTCGTGAA |
| | GCTGAGTCAGCGCATTTTGGGACCCTGTACCATTGTCCAGGGAGCTCTGAAAAGCATC |
| | CTATGTCGCACCCCGGGAGAGTTTTACCACAACACTCTGAGCTTCCTCAAGTCCAATG |
| | CTGATCTCTGTTATGGGGCGTTGGCTGCCATCCCTGGACTCCGGCCAGTCCGCCCTTC |
| | TGGGGCTATGTACCTCATGGTTGGAATTGAGATGGAACATTTCCCAGAATTTGAGAAC |
| | GATGTGGAGTTCACGGAGCGGTTAGTTGCTGAGCAGTCTGTCCACTGCCTCCCAGCAA |
| | CGTGCTTTGAGTACCCGAATTTCATCCGAGTGGTCATCACAGTCCCCGAGGTGATGAT |
| | GCTGGAGGCGTGCAGCCGGATCCAGGAGTTCTGTGAGCAGCACTACCATTGTGCTGAA |
| | GGCAGCCAGGAGGAGTGTGATAAACATCATCACCACCATCACTAGGCGCCCGCACTCG |
| | AGGACCACCACCACCACCAC |
| | ORF Start: ATG at 9 ORF Stop: TAG at 1377 |
| | SEQ ID NO: 166 456 aa MW at 50715.lkD |
| NOV12l, | MIQMSSKGNLPSILDVHVNVGGRSSVPGKMKGRKARWSVRPSDMAKKTFNPIRAIVDN |
| CG135823-04 | |
| Protein Sequence | MKVKPNPNKTMISLSIGDPTVFGNLPTDPEVTQAMKDALDSGKYNGYAPSIGFLSSRE |
| | EIASYYHCPEAPLEAKDVILTSGCSQAIDLCLAVLANPGQNILVPRPGFSLYKTLAES |
| | MGIEVKLYNLLPEKSWEIDLKQLEYLIDEKTACLIVNNPSNPCGSVFSKRHLQKILAV |
| | AARQCVPILADEIYGDMVFSDCKYEPLATLSTDVPILSCGGLAKRWLVPGWRLGWILI |
| | HDRRDIFGNEIRDGLVKLSQRILGPCTIVQGALKSILCRTPGEFYHNTLSFLKSNADL |
| | CYGALAAIPGLRPVRPSGAMYLMVGIEMEHFPEFENDVEFTERLVAEQSVHCLPATCF |
| | EYPNFIRVVITVPEVMMLEACSRIQEFCEQHYHCAEGSQEECDKHNHHHH |

Sequence comparison of the above protein sequences yields the following sequence relationships shown in Table 12B.

TABLE 12B
Comparison of NOV12a against NOV12b through NOV121.

| | | Identities/ |
|---|---|---|
| | | Similarities |
| Protein | NOV12a Residues/ | for the |
| Sequence | Match Residues | Matched Region |
| NOV12b | 1 . . . 454 | 454/454 (100%) |
| | 1 . . . 454 | 454/454 (100%) |
| NOV12c | 1 . . . 454 | 454/454 (100%) |
| | 5 . . . 458 | 454/454 (100%) |
| NOV12d | 1 . . . 454 | 411/454 (90%) |
| | 5 . . . 415 | 411/454 (90%) |
| NOV12e | 1 . . . 454 | 454/454 (100%) |
| | 2 . . . 454 | 454/454 (100%) |
| NOV12f | 3 . . . 454 | 452/452 (100%) |
| | 1 . . . 452 | 452/452 (100%) |
| NOV12g | 2 . . . 454 | 453/453 (100%) |
| | 7 . . . 459 | 453/453 (100%) |
| NOV12h | 3 . . . 454 | 452/452 (100%) |
| | 1 . . . 452 | 452/452 (100%) |
| NOV12i | 1 . . . 454 | 454/454 (100%) |
| | 1 . . . 454 | 454/454 (100%) |
| NOV12j | 1 . . . 454 | 454/454 (100%) |
| | 2 . . . 455 | 454/454 (100%) |
| NOV12k | 2 . . . 454 | 453/453 (100%) |
| | 7 . . . 459 | 453/453 (100%) |
| NOV12l | 5 . . . 454 | 450/450 (100%) |
| | 1 . . . 450 | 450/450 (100%) |

Further analysis of the NOV12a protein yielded the following properties shown in Table 12C.

TABLE 12C
Protein Sequence Properties NOV12a

| | |
|---|---|
| PSort analysis: | 0.6500 probability located in cytoplasm; |
| | 0.1000 probability located in mitochondrial |
| | matrix space; 0.1000 probability located in |
| | lysosome (lumen); 0.0000 probability located |
| | in endoplasmic reticulum (membrane) |
| SignalP analysis: | No Known Signal Sequence Predicted |

A search of the NOV12a protein against the Geneseq database, a proprietary database that contains sequences published in patents and patent publication, yielded several homologous proteins shown in Table 12D.

TABLE 12D
Geneseq Results for NOV12a

| | | | Identities/ | |
|---|---|---|---|---|
| | | | Similarities | |
| Geneseq | Protein/Organism/ | NOV12a Residues/ | for the | Expect |
| Identifier | Length [Patent #, Date] | Match Residues | Matched Region | Value |
| ABB58136 | Drosophila melanogaster | 37 . . . 442 | 212/411 (51%) | e−128 |
| | polypeptide SEQ ID NO 1200 - | 75 . . . 481 | 296/411 (71%) | |
| | Drosophila melanogaster, 501 aa. | | | |
| | [WO200171042-A2, 27 SEP. 2001] | | | |
| AAG10932 | Arabidopsis thaliana protein | 68 . . . 441 | 136/382 (35%) | 3e−67 |
| | fragment SEQ ID NO: 9454 - | 8 . . . 385 | 220/382 (56%) | |
| | Arabidopsis thaliana, 407 aa. | | | |
| | [EP1033405-A2, 06 SEP. 2000] | | | |
| AAG10931 | Arabidopsis thaliana protein | 68 . . . 441 | 136/382 (35%) | 3e−67 |
| | fragment SEQ ID NO: 9453 - | 46 . . . 423 | 220/382 (56%) | |
| | Arabidonsis thaliana. 445 aa. | | | |
| | [EP1033405-A2, 06 SEP. 2000] | | | |
| AAG10930 | Arabidopsis thaliana protein | 68 . . . 441 | 136/382 (35%) | 3e−67 |
| | fragment SEQ ID NO: 9452 - | 67 . . . 444 | 220/382 (56%) | |
| | Arabidopsis thaliana, 466 aa. | | | |
| | [EP1033405-A2, 06 SEP. 2000] | | | |
| AAG39068 | Arabidopsis thaliana protein | 68 . . . 441 | 135/382 (35%) | 3e−66 |
| | fragment SEQ ID NO: 48288 - | 8 . . . 385 | 219/382 (56%) | |
| | Arabidopsis thaliana, 407 aa. | | | |
| | [EP1033405-A2, 06 SEP. 2000] | | | |

In a BLAST search of public sequence datbases, the NOV12a protein was found to have homology to the proteins shown in the BLASTP data in Table 12E.

TABLE 12E
Public BLASTP Results for NOV12a

| | | | Identities/ | |
|---|---|---|---|---|
| Protein | | | Similarities | |
| Accession | | NOV12a Residues/ | for the | Expect |
| Number | Protein/Organism/Length | Match Residues | Matched Portion | Value |
| P17735 | Tyrosine aminotransferase (EC | 1 . . . 454 | 454/454 (100%) | 0.0 |
| | 2.6.1.5) (L-tyrosine: 2-oxoglutarate | 1 . . . 454 | 454/454 (100%) | |
| | aminotransferase) (TAT) - Homo | | | |
| | sapiens (Human), 454 aa. | | | |
| Q8QZR1 | Similar to tyrosine aminotransferase | 1 . . . 454 | 418/454 (92%) | 0.0 |
| | (Hypothetical 50.6 kDa protein) - | 1 . . . 454 | 439/454 (96%) | |
| | Mus musculus (Mouse), 454 aa. | | | |
| P04694 | Tyrosine aminotransferase (EC | 1 . . . 454 | 416/454 (91%) | 0.0 |
| | 2.6.1.5) (L-tyrosine: 2-oxoglutarate | 1 . . . 454 | 436/454 (95%) | |
| | aminotransferase) (TAT) - Rattus | | | |
| | norvegicus (Rat), 454 aa. | | | |
| Q9XSW4 | Tyrosine aminotransferase - | 1 . . . 454 | 417/454 (91%) | 0.0 |
| | Mustela vison (American mink), | 1 . . . 454 | 438/454 (95%) | |
| | 454 aa. | | | |
| Q9QWS4 | Tyrosine aminotransferase - | 1 . . . 454 | 415/454 (91%) | 0.0 |
| | Rattus norvegicus (Rat), 454 aa. | 1 . . . 454 | 435/454 (95%) | |

PFam analysis predicts that the NOV12a protein contains the domains shown in the Table 12F.

TABLE 12F
Domain Analysis of NOV12a

| | | Identities/ | |
|---|---|---|---|
| | | Similarities | |
| | NOV12a Match | for the | Expect |
| Pfam Domain | Region | Matched Region | Value |
| aminotran_1_2 | 113 . . . 438 | 72/356 (20%) | 2.1e−76 |
| | | 262/356 (74%) | |

### Example 13

The NOV13 clone was analyzed, and the nucleotide and encoded polypeptide sequences are shown in Table 13A.

TABLE 13A
NOV13 Sequence Analysis

| | |
|---|---|
| | SEQ ID NO: 167 1894 bp |
| NOV13a, | CGCCGCTCGCCCCAGACTTACTTCCCCGGCTCACCAGGGAAAGGTTCCTAGAAGGTGA |
| CG140122-01 | |
| DNA Sequence | GCGCGGACGGTATGCAAAGTTGTGAATCCAGTGGTGACAGTGCGGATGACCCTCTCAG |
| | TCGCGGCCTACGGAGAAGGGGACAGCCTCGTGTGGTGGTGATCGGCGCCGGCTTGGCT |
| | GGCCTGGCTGCAGCCAAAGCACTTCTTGAGCAGGGTTTCACGGATGTCACTGTGCTTG |
| | AGGCTTCCAGCCACATCGGAGGCCGTGTGCAGAGTGTGAAACTTGGACACGCCACCTT |
| | TGAGCTGCGAGCCACCTGGATCCATGGCTCCCATGGGAACCCTATCTATCATCTAGCA |
| | GAAGCCAACGAACCTCCTGGAAGAGACAACCGATGGGGAACGCAGCGTGGGCCGCATCA |
| | GCCTCTATTCCAAGAATGGCGTGGCCTGCTACCTTACCAACCACGGCCGCAGGATCCC |
| | CAAGGACGTGGTTGAGGAATTCAGCGATTTATACAACGAGGTCTATAACTTGACCCAG |
| | GAGTTCTTCCGGCACGATAAACCAGTCAATGCTGAAAGTCAAAATAGCGTGGGGGTGT |
| | TCACCCGAGAGGACGTGCGTAACCGCATCAGGAATGACCCTGACGACCCAGAGGCTAC |
| | CAAGCGCCTGAAGCTCGCCATGATCCAGCAGTACCTGAAGGTGGAGAGCTGTGAGAGC |
| | AGCTCACACAGCATGGACGAGGTGTCCCTGAGCGCCTTCGGGGAGTGGACCGAGATCC |
| | CCGGCGCTCACCACATCATCCCCTCGGGCTTCATGCGGGTTGTGGAGCTGCTGGCGGA |
| | GGGCATCCCTGCCCACGTCATCCAGCTAGGGAAACCTGTCCGCTGCATTCACTGGGAC |
| | CAGGCCTCAGCCCGCCCCAGAGGCCCTGAGATTGAGCCCCGGGGTGAGGGCGACCACA |
| | ATCACGACACTGGGGAGGGTGGCCAGGGTGGAGAGGAGCCCCCGGGGGGCAGGTGGGA |
| | TGAGGATGAGCAGTGGTCGGTGGTGGTGGAGTGCGAGGACCGTGAGCTGATCCCGGCG |
| | GACCATGTGATTGTGACCGTGTCGCTAGGTGTGCTAAAGAGGCAGTACACCAGTTTCT |
| | TCCGCCCAGGCCTGCCCACAGAGAAGGTGGCTGCCATCCACCCCCTGGGCATTGGCAC |
| | CACCGACAAGATCTTTCTGGAATTCGAGGAGCCCTTCTGGGGCCCTGAGTGCAACAGC |
| | CTACAGTTTGTGTGGGAGGACGAAGCGGAGAGCCACACCCTCACCTACCCACCTGAGC |
| | TCTGGTACCGCAAGATCTGCGGCTTTGATGTCCTCTACCCGCCTGAGCGCTACGGCCA |
| | TGTGCTGAGCGGCTGGATCTGCGGGGAGGAGGCCCTCGTCATGGAGAAGTGTGATGAC |
| | GAGGCAGTGGCCGAGATCTGCACGGAGATGCTGCGTCAGTTCACAGGGAACCCCAACA |
| | TTCCAAAACCTCGGCGAATCTTGCGCTCGGCCTGGGGCAGCAACCCTTACTTCCGTGG |
| | CTCCTATTCATACACGCAGGTGGGCTCCAGCGGGGCGGATGTGGAGAAGCTGGCCAAG |
| | CCCCTGCCGTACACGGAGAGCTCAAAGACAGCGCCCATGCAGGTGCTGTTTTCCGGTG |
| | AGGCCACCCACCGCAAGTACTATTCCACCACCCACGGTGCTCTGCTGTCCGGCCAGCG |
| | TGAGGCTGCCCGCCTCATTGAGATGTACCGAGACCTCTTCCAGCAGGGGACCTGAGGG |
| | CTGTCCTCGCTGCTGAGAAGAGCCACTAACTCGTGACCTCCAGCCTGCCCCTTGCTGC |
| | CGTGTGCTCCTGCCTTCCTGATCCTCTGTAGAAAGGATTTTTATCTTCTGTAGAGCTA |
| | GCCGCCCTGACTGCCTTCAGACCTGGCCCTGTAGCTTT |
| | ORF Start: ATG at 70 ORF Stop: TGA at 1735 |
| | SEQ ID NO: 168 555 aa MW at 61871.7kD |
| NOV13a, | MQSCESSGDSADDPLSRGLRRRGQPRVVVIGAGLAGLAAAKALLEQGFTDVTVLEASS |
| CG140122-01 | |
| Protein Sequence | HIGGRVQSVKLGHATFELGATWIHGSHGNPIYHLAEANGLLEETTDGERSVGRISLYS |
| | KNGVACYLTNHGRRIPKDVVEEFSDLYNEVYNLTQEFFRHDKPVNAESQNSVGVFTRE |
| | EVRNRIRNDPDDPEATKRLKLANIQQYLKVESCESSSHSMDEVSLSAFGEWTEIPGAII |
| | HIIPSGFMRVVELLAEGIPAHVIQLGKPVRCIHWDQASARPRGPEIEPRGEGDHNHDT |
| | GEGGQGGEEPRGGRWDEDEQWSVVVECEDRELIPADHVIVTVSLGXTLKRQYTSFFRPG |
| | LPTEKVAAIHRLGIGTTDKIFLEFEEPFWGPECNSLQFVWEDEAESHTLTYPPELWYR |
| | KICGFDVLYPPERYGHVLSGWICGEEALVMEKCDDEAVAEICTEMLRQFTGNPNIPKP |
| | RRILRSAWGSNPYFRGSYSYTQVGSSGADVEKLAKPLPYTESSKTAPMQVLFSGEATH |
| | RKYYSTTNGALLSGQREAARLIEMYRDLFQQGT |
| | SEQ ID NO: 169 1012 bp |
| NOV13b, | CACCATGCAAAGTTGTGAATCCAGTGGTGACAGTGCGGATGACCCTCTCAGTCGCGGC |
| 246864043 | |
| DNA Sequence | CTACGGAGAAGGGGACAGCCTCGTGTGGTGGTGATCGGCGCCGGCTTGGCTGGCCTGG |
| | CTGCAGCCAAAGCACTTCTTGAGCAGGGTTTCACGGATGTCACTGTGCTTGAGGCTTC |
| | CAGCCACATCGGAGGCCGTGTGCAGAGTGTGAAACTTGGACACGCCACCTTTGAGCTG |
| | GGAGCCACCTGGATCCATGGCTCCCATGGGAACCCTATCTATCATCTAGCAGAAGCCA |
| | ACGGCCTCCTGGAAGAGACAACCGATGGGGAACGCAGCGTGGGCCGCATCAGCCTCTA |
| | TTCCAAGAATGGCGTGGCCTGCTACCTTACCAACCACGGCCGCAGGATCCCCAAGGAC |
| | GTGGTTGAGGAATTCAGCGATTTATACAACGAGGTCTATAACTTGACCCAGGAGTTCT |
| | TCCGGCACGATAAACCAGTCAATGCTGAAAGTCAAAATAGCGTGGGGGTGTTCACCCG |
| | AGAGGAGGTGCGTAACCGCATCAGGAATGACCCTGACGACCCAGAGGCTACCAAGCGC |
| | CTGAAGCTCGCCATGATCCAGCAGTACCTGAAGGTGGAGAGCTGTGAGAGCAGCTCAC |
| | ACAGCATGGACGAGGTGTCCCTGAGCGCCTTCGGGGAGTGGACCGAGATCCCCGGCGC |
| | TCACCACATCATCCCCTCGGGCTTCATGCGGGTTGTGGAGCTGCTGGCGGAGGGCATC |
| | CCTGCCCACGTCATCCAGCTAGGGAAACCTGTCCGCTGCATTCACTGGGACCAGGCCT |
| | CAGCCCGCCCCAGAGGCCCTGAGATTGAGCCCCTGCCGTACACAGAGAGCTCAAAGAC |
| | AGCGCCCATGCAGGTGCTGTTTTCCGGTGAGGCCACCCACCGCAAGTACTATTCCACC |
| | ACCCACGGTGCTCTGCTGTCCGGCCAGCGTGAGGCTGCCCGCCTCATTGAGATGTACC |
| | GAGACCTCTTCCAGCAGGGGACCTGA |
| | ORF Start: at 2 ORF Stop: TGA at 1010 |
| | SEQ ID NO: 170 336 aa MW at 37093.2kD |
| NOV13b, | TMQSCESSGDSADDPLSRGLRRRGQPRVVVIGAGLAGLAAAKALLEQGFTDVTVLEAS |
| 246864043 | |
| Protein Sequence | SHIGGRVQSVKLGHATFELGATWIHGSHGNPIYHLAEANGLLEETTDGERSVGRISLY |
| | SKNGVACYLTNHGRRIPKDVVEEFSDLYNEVYNLTQEFFRHDKPVNAESQNSVGVFTR |
| | EEVRNRIRNDPDDPEATKRLKLAMIQQYLKVESCESSSHSMDEVSLSAFGEWTEIPGA |
| | HHIIPSGFMRVVELLAEGIPAHVIQLGKPVRCIHWDQASARPRGPEJIEPLPYTESSKT |
| | APMQVLFSGEATHRKYYSTTHGALLSGQREAARLIEMYRDLFQQGT |
| | SEQ ID NO: 171 1603 bp |
| NOV13c, | CACCATGCAAAGTTGTGAATCCAGTGGTGACAGTGCGGATGACCCTCTCAGTCGCGGC |
| 246864086 | |
| DNA Sequence | CTACGGAGAAGGGGACAGCCTCGTGTGGTGGTGATCGGCGCCGGCTTGGCTGGCCTCG |
| | CTGCAGCCAAAGCACTTCTTGAGCAGGGTTTCACGGATGTCACTGTGCTTGAGGCTTC |
| | CAGCCACATCGGAGGCCGTGTGCAGAGTGTGAAACTTGGACACGCCACCTTTGAGCTG |
| | GGAGCCACCTGGATCCATGGCTCCCATGCGAACCCTATCTATCATCTAGCAGAAGCCA |
| | ACGGCCTCCTGGAAGAGACAACCGATGGCGAACGCAGCGTGGGCCGCATCAGCCTCTA |
| | TTCCAAGAATGGCGTGGCCTGCTACCTTACCAACCACGGCCGCAGGATCCCCAAGGAC |
| | GTGGTTGAGGAATTCAGCGATTTATACAACGAGGTCTATAACTTGACCCAGGAGTTCT |
| | TCCGGCACGATAAACCAGTCAATGCTGAAAGTCAAAATAGCGTGGGGGTGTTCACCCG |
| | AGAGGAGGTGCGTAACCGCATCAGGAATGACCCTGACGACCCAGAGGCTACCAAGCGC |
| | CTGAAGCTCGCCATGATCCAGCAGTACCTGAAGGTGCACAGCTGTGAGAGCAGCTCAC |
| | ACAGCATGGACGAGGTGTCCCTCAGCGCCTTCGGGGAGTGGACCGAGATCCCCGGCGC |
| | TCACCACATCATCCCCTCGGGCTTCATGCGGGTTGTGGAGCTGCTGGCGGAGGGCATC |
| | CCTGCCCACGTCATCCAGCTAGGGAAACCTGTCCGCTGCATTCACTGGGACCAGGCCT |
| | CAGCCCGCCCCAGAGGCCCTGAGATTGAGCCCCGGGGTGTGCTAAAGAGGCAGTACAC |
| | CAGTTTCTTCCGGCCAGGCCTGCCCACAGAGAAGGTGGCTGCCATCCACCGCCTGGGC |
| | ATTGGCACCACCGACAAGATCTTTCTGGAATTCGAGGAGCCCTTCTGGGGCCCTGAGT |
| | GCAACAGCCTACAGTTTGTGTGGGAGGACGAAGCGGACAGCCACACCCTCACCTACCC |
| | ACCTGAGCTCTGGTACCGCAAGATCTGCGGCTTTGATGTCCTCTACCCGCCTGAGCGC |
| | TACGGCCATGTGCTGAGCGGCTGGATCTGCGGGGAGGAGGCCCTCGTCATGGAGAAGT |
| | GTGATGACGAGGCAGTGGCCGAGATCTGCACGGAGATGCTGCGTCAGTTCACAGOGAA |
| | CCCCAACATTCCAAAACCTCGGCGAATCTTGCGCTCGGCCTGGGGCAGCAACCCTTAC |
| | TTCCGCGGCTCCTATTCATACACGCAGGTGGGCTCCAGCGGGGCGGATGTGGAGAAGC |
| | TGGCCAAGCCCCTGCCGTACACGGAGAGCTCAAAGACAGCGCATGGAAGCTCCACAAA |
| | GCAGCAGCCTGGTCACCTTTTCTCTTCCAAGTGCCCAGAACAGCCCCTGGATGCTAAC |
| | AGGGGCGCCGTAAAGCCCATGCAGGTGCTGTTTTCCGGTGAGGCCACCCACCGCAAGT |
| | ACTATTCCACCACCCACGGTGCTCTGCTGTCCGGCCAGCGTGAGGCTGCCCGCCTCAT |
| | TGAGATGTACCGAGACCTCTTCCAGCAGGGGACCTGA |
| | ORF Start: at 2 ORF Stop: TGA at 1601 |
| | SEQ ID NO: 172 533 aa MW at 59379.2kD |
| NOV13c, | TMQSCESSGDSADDPLSRGLRRRGQPRVVVIGAGLAGLAAAKALLEQGFTDVTVLEAS |
| 246864086 | |
| Protein Sequence | SHIGGRVQSVKLGHATFELGATWIHGSHGNPIYHLAEANGLLEETTDGERSVGRISLY |
| | SKNGVACYLTNhGRRIPKDVVEEFSDLYNEVYNLTQEFFRhDKPVNAESQNSVGVFTR |
| | EEVRNRIRNDPDDPEATKRLKLAMIQQYLKVESCESSSHSMDEVSLSAFGEWTEIPGA |
| | HHIIPSGFMRVVELLAEGIPAHVIQLGKPVRCIHWDQASARPRGPEIEPRGVLKRQYT |
| | SFFRPGLPTEKVAAIHRLGIGTTDKIFLEFEEPFWGPECNSLQFVWEDEAESHTLTYP |
| | PELWYRKICGFDVLYPPERYGHVLSGWICGEEALVMEKCDDEAVAEICTEMLRQFTGN |
| | PNIPKPRRILRSAWGSNPYFRGSYSYTQVGSSGADVEKLAKPLPYTESSKTAHGSSTK |
| | QQPGHLFSSKCPEQPLDANRGAVKPMQVLFSGEATHRKYYSTTHGALLSGQREAARLI |
| | EMYRDLFQQGT |
| | SEQ ID NO: 173 1693 bp |
| NOV13d, | CACCATGGGACATCATCACCACCATCACCAAAGTTGTGAATCCAGTGGTGACAGTGCG |
| 258280083 | |
| DNA Sequence | GATGACCCTCTCAGTCGCGGCCTACGGAGAAGGGGACAGCCTCGTGTGGTGGTGATCG |
| | GCGCCGGCTTGGCTGGCCTGGCTGCAGCCAAAGCACTTCTTGAGCAGGGTTTCACGCA |
| | TGTCACTGTGCTTGAGGCTTCCAGCCACATCGGAGGCCGTGTGCAGAGTGTGAAACTT |
| | GGACACGCCACCTTTGAGCTGGGAGCCACCTGGATCCATGGCTCCCATGGGAACCCTA |
| | TCTATCATCTAGCAGAAGCCAACGGCCTCCTGGAAGAGACAACCGATGGGGAACGCAG |
| | CGTGGGCCGCATCAGCCTCTATTCCAAGAATGGCGTGGCCTGCTACCTTACCAACCAC |
| | GGCCGCAGGATCCCCAAGGACGTGGTTGAGGAATTCAGCGATTTATACAACGAGGTCT |
| | ATAACTTGACCCAGGAGTTCTTCCGGCACGATAAACCAGTCAATGCTGAAAGTCAAAA |
| | TAGCGTGGGGGTGTTCACCCGACAGGAGGTGCGTAACCGCATCAGGAATGACCCTGAC |
| | GACCCAGAGGCTACCAAGCGCCTGAAGCTCGCCATGATCCAGCAGTACCTGAAGGTGG |
| | AGAGCTGTGAGAGCAGCTCACACAGCATGGACGAGGTGTCCCTGAGCGCCTTCGGGGA |
| | GTGGACCGAGATCCCCGGCGCTCACCACATCATCCCCTCGGGCTTCATGCGGGTTGTG |
| | GAGCTGCTGGCGGAGGGCATCCCTGCCCACGTCATCCAGCTAGGGAAACCTGTCCGCT |
| | GCATTCACTGGGACCAGGCCTCAGCCCGCCCCAGAGGCCCTGAGATTGAGCCCCGGGG |
| | TGAGGGCGACCACAATCACGACACTGGGGAGGGTGGCCAGGGTGCAGAGGAGCCCCGG |
| | GGGGGCAGGTGGGATGAGGATGAGCAGTGGTCGGTGGTGGTGGAGTGCGAGGACTGTG |
| | AGCTGATCCCGGCGGACCATGTGATTGTGACCGTGTCGCTAGGTGTGCTAAAGAGGCA |
| | GTACACCAGTTTCTTCCGGCCAGGCCTGCCCACAGAGAAGGTGGCTGCCATCCACCGC |
| | CTGGGCATTGGCACCACCGACAAGATCTTTCTGGAATTCGAGGACCCCTTCTGGGGCC |
| | CTGAGTGCAACAGCCTACAGTTTGTGTGGGAGGACGAAGCAGAGAGCCACACCCTCAC |
| | CTACCCACCTGAGCTCTGGTACCGCAAGATCTGCGGCTTTGATGTCCTCTACCCGCCT |
| | GAGCGCTACGGCCATGTGCTGAGCGGCTGGATCTGCGGGGAGGAGGCCCTCGTCATGG |
| | AGAAGTGTGATGACGACGCAGTGGCCGAGATCTGCACGGAGATGCTGCGTCAGTTCAC |
| | AGGGAACCCCAACATTCCAAAACCTCGGCGAATCTTGCGCTCGGCCTGGGGCAGCAAC |
| | CCTTACTTCCGCGGCTCCTATTCATACACGCAGGTGGGCTCCAGCGGGGCGGATGTGG |
| | AGAAGCTGGCCAAGCCCCTGCCGTACACGGAGAGCTCAAAGACAGCGCCCATGCAGGT |
| | GCTGTTTTCCGGTGAGGCCACCCACCGCAAGTACTATTCCACCACCCACGGTGCTCTG |
| | CTGTCCGGCCAGCGTGAGGCTGCCCGCCTCATTGAGATGTACCGAGACCTCTTCCAGC |
| | AGGGGACCTGA |
| | ORF Start: at 2 ORF Stop: TGA at 1691 |
| | SEQ ID NO: 174 563 aa MW at 62799.6kD |
| NOV13d, | TMGHHHHHHQSCESSGDSADDPLSRGLRRRGQPRVVVIGAGLAGLAAAKALLEQGFTD |
| 258280083 | |
| Protein Sequence | VTVLEASSHIGGRVQSVKLGHATFELGATWIHGSHGNPIYHLAEANGLLEETTDGERS |
| | VGRISLYSKNGVACYLTNHGRRIPKDVVEEFSDLYNEVYNLTQEFFRHDKPVNAESQN |
| | SVGVFTREEVRNRIRNDPDDPEATKRLKLANIQQYLKVESCESSSHSMDEVSLSAFGE |
| | WTEIPGAHHIIPSGFMRVVELLAEGIPAHVIQLGKPVRCIHWDQASARPRGPEIEPRG |
| | EGDHNHDTGEGGQGGEEPRGGRWDEDEQWSVVVECEDCELIPADHVIVTVSLGVLKRQ |
| | YTSFFRPGLPTEKVAAIHRLGIGTTDKIFLEFEEPFWGPECNSLQFVWEDEAESHTLT |
| | YPPELWYRKICGFDVLYPPERYGHVLSGWICGEEALVMEKCDDEAVAEICTEMLRQFT |
| | GNPNIPKPRRILRSAWGSNPYFRGSYSYTQVGSSGADVEKLAKPLPYTESSKTAPMQV |
| | LFSGEATHRKYYSTTHGALLSGQREAARLIEMYRDLFQQGT |
| | SEQ ID NO: 175 1672 bp |
| NOV13e, | CACCATGCAAAGTTGTGAATCCAGTGGTGACAGTGCGGATGACCCTCTCAGTCGCGGC |
| 258280066 | |
| DNA Sequence | CTACGGAGAAGGGGACAGCCTCGTGTGGTGGTGATCGGCGCCGGCTTGGCTGCCCTGG |
| | CTGCAGCCAAAGCACTTCTTGAGCAGGGTTTCACGGATGTCACTGTGCTTGAGGCTTC |
| | CAGCCACATCGGAGGCCGTGTGCAGAGTGTGAAACTTGGACACGCCACCTTTGAGCTG |
| | GGAGCCACCTGGATCCATGGCTCCCATGGGAACCCTATCTATCATCTAGCAGAAGCCA |
| | ACGGCCTCCTGGAAGAGACAACCGATGGGGAACGCAGCGTGGGCCGCATCAGCCTCTA |
| | TTCCAAGAATGGCGTGGcCTGCTACCTTACCAACCACGGCCGCAGGATCCCCAAGGAc |
| | GTGGTTGAGGAATTCAGCGATTTATACAACGAGGTCTATAACTTGACCCAGGAGTTCT |
| | TCCGGCACGATAAACCAGTCAATGCTGAAAGTCAAAATAGCGTGGGGGTGTTCACCCG |
| | AGAGGAGGTGCGTAACCGCATCAGGAATGACCCTGACGACCCAGAGGCTACCAAGCGC |
| | CTGAAGCTCGCCATGATCCAGCAGTACCTGAAGGTGGAGAGCTGTGAGAGCAGCTCAC |
| | ACAGCATGGACGAGGTGTCCCTGAGCGCCTTCGGGGAGTGGACCGAGATCCCCGGCGC |
| | TCACCACATCATCCCCTCGGGCTTCATGCGGGTTGTGGAGCTGCTGGCGGAGGGCATC |
| | CCTGCCCACGTCATCCAGCTAGGGAAACCTGTCCGCTGCATTCACTGGGACCAGGCCT |
| | CAGCCCGCCCCAGAGGCCCTGAGATTGAGCCCCGGGGTGAGGGCGACCACAATCACGA |
| | CACTGGGGAGGGTGGCCAGGGTGGAGAGGAGCCCCGGGGGGGCAGGTGGGATGAGGAT |
| | GAGCAGTGGTCGGTGGTGGTGGAGTGCGAGGACTGTGAGCTGATCCCGGCGGACCATG |
| | TGATTGTGACCGTGTCGCTAGGTGTGCTAAAGAGGCAGTACACCAGTTTCTTCCGGCC |
| | AGGCCTGCCCACAGAGAAGGTGGCTGCCATCCACCGCCTGGGCATTGGCACCACCGAC |
| | AAGATCTTTCTGCAATTCGAGGAGCCCTTCTGGGGCCCTGAGTGCAACAGCCTACAGT |
| | TTGTGTGGGAGGACGAAGCAGAGAGCCACACCCTCACCTACCCACCTGAGCTCTGGTA |
| | CCGCAAGATCTGCGGCTTTGATGTCCTCTACCCGCCTGAGCGCTACGGCCATGTGCTG |
| | AGCGGCTGGATCTGCGGGGAGGAGGCCCTCGTCATGGAGAAGTGTGATGACGAGGCAG |
| | TGGCCGACATCTGCACGGAGATGCTGCGTCAGTTCACAGGGAACCCCAACATTCCAAA |
| | ACCTCGGCGAATCTTGCGCTCGGCCTGGGGCAGCAACCCTTACTTCCGCGGCTCCTAT |
| | TCATACACGCAGGTGGGCTCCAGCGGGGCGGATGTGGAGAAGCTGGCCAAGCCCCTGC |
| | CGTACACGGAGAGCTCAAAGACAGCGCCCATGCAGGTGCTGTTTTCCGGTGAGGCCAC |
| | CCACCGCAAGTACTATTCCACCACCCACGGTGCTCTGCTGTCCGGCCAGCGTGAGGCT |
| | GCCCGCCTCATTGAGATGTACCGAGACCTCTTCCAGCAGGGGACCTGA |
| | ORF Start: at 2 ORF Stop: TGA at 1670 |
| | SEQ ID NO: 176 556 aa MW at 61919.7kD |
| NOV13e, | TMQSCESSGDSADDPLSRGLRRRGQPRVVVIGAGLAGLAAAKALLEQGFTDVTVLEAS |
| 258280066 | |
| Protein Sequence | SHIGGRVQSVKLGHATFELGATWIHGSHGNPIYHLAEANGLLEETTDGERSVGRISLY |
| | SKNGVACYLTNHGRRIPKDVVEEFSDLYNEVYNLTQEFFRHDKPVNAESQNSVGVFTR |
| | EEVRNRIRNDPDDPEATKRLKLANIQQYLKVESCESSSHSMDEVSLSAFGEWTEIPGA |
| | HHITPSGFMRVVELLAEGIPAHVIQLGKPVRCIHWDQASARPRGPEIEPRGEGDHNHD |
| | TGEGGQGGEEPRGGRWDEDEQWSVVVECEDCELIPADHVIVTVSLGVLKRQYTSFFRP |
| | CLPTEKVAAIHRLGIGTTDKIFLEFEEPFWGPECNSLQFVWEDEAESHTLTYPPELWY |
| | RKICGFDVLYPPERYGHVLSGWICGEEALVMEKCDDEAVAEICTEMLRQFTGIJPNT PK |
| | PRRILRSAWGSNPYFRGSYSYTQVGSSGADVEKLAKPLPYTESSKTAPMQVLFSGEAT |
| | HRKYYSTTHGALLSGQREAARLIEMYRDLFQQGT |
| | SEQ ID NO: 177 1690 bp |
| NOV13f | CACCATGCAAAGTTGTGAATCCAGTGGTGACAGTGCGGATGACCCTCTCAGTCGCGGC |
| 258329988 | |
| DNA Sequence | CTACGGAGAAGGGGACAGCCTCGTGTGGTGGTGATCGGCGCCGGCTTGGCTGGCCTGG |
| | CTGCAGCCAAAGCACTTCTTGAGCAGGGTTTCACGGATGTCACTGTGCTTGAGGCTTC |
| | CAGCCACATCGGAGGCCGTGTGCAGAGTGTGAAACTTOGACACGCCACCTTTGAGCTG |
| | GGAGCCACCTGGATCCATGGCTCCCATGGGAACCCTATCTATCATCTAGCAGAAGCCA |
| | ACGGCCTCCTGGAAGAGACAACCGATCGGGAACGCAGCGTGGGCCGCATCAGCCTCTA |
| | TTCCAAGAATGGCGTGGCCTGCTACCTTACCAACCACGGCCGCAGGATCCCCAAGGAC |
| | GTGGTTGAGGAATTCAGCGATTTATACAACGAGGTCTATAACTTGACCCAGGAGTTCT |
| | TCCGGCACGATAAACCAGTCAATGCTGAAAGTCAAAATAGCGTGGGGGTGTTCACCCG |
| | AGAGGAGGTGCGTAACCGCATCAGGAATGACCCTGACGACCCAGAGGCTACCAAGCGC |
| | CTGAAGCTCGCCATGATCCAGCAGTACCTGAAGGTGGAGAGCTGTGAGAGCAGCTCAC |
| | ACAGCATGGACGAGGTGTCCCTGAGCGCCTTCGGGGAGTGGACCGAGATCCCCGGCGC |
| | TCACCACATCATCCCCTCGGGCTTCATGCGGGTTGTGGAGCTGCTGGCGGAGGGCATC |
| | CCTGCCCACGTCATCCAGCTAGGGAAACCTGTCCGCTGCATTCACTGGGACCAGGCCT |
| | CAGCCCGCCCCAGAGGCCCTGAGATTGAGCCCCGGGGTGAGGGCGACCACAATCACGA |
| | CACTGGGGAGGGTGGCCAGGGTCGAGAGGAGCCCCGGGGGGGCAGGTGGGATGAGGAT |
| | GAGCAGTGGTCGGTGGTGGTGGAGTGCGAGGACTGTGAGCTGATCCCGGCGGACCATG |
| | TGATTGTGACCGTGTCGCTAGGTGTGCTAAAGAGGCAGTACACCAGTTTCTTCCGGCC |
| | AGGCCTGCCCACAGAGAAGGTGGCTGCCATCCACCGCCTGGGCATTGGCACCACCGAC |
| | AAGATCTTTCTGGAATTCGAGGAGCCCTTCTGGGGCCCTCAGTGCAACAGCCTACAGT |
| | TTGTGTGGGAGGACGAAGCAGAGAGCCACACCCTCACCTACCCACCTGAGCTCTGGTA |
| | CCGCAAGATCTGCGGCTTTGATGTCCTCTACCCGCCTGAGCGCTACGGCCATGTGCTG |
| | AGCGGCTGGATCTGCGGGGAGGAGGCCCTCGTCATGGAGAAGTGTGATGACGAGGCAG |
| | TGGCCGAGATCTGCACGGAGATGCTGCGTCAGTTCACAGGGAACCCCAACATTCCAAA |
| | ACCTCGGCGAATCTTGCGCTCGGCCTGGGGCAGCAACCCTTACTTCCGCGGCTCCTAT |
| | TCATACACGCAGGTGGGCTCCAGCGGGGCGGATGTGGAGAAGCTGGCCAAGCCCCTGC |
| | CGTACACGGAGAGCTCAAAGACAGCGCCCATGCAGGTGCTGTTTTCCGGTGAGGCCAC |
| | CCACCGCAAGTACTATTCCACCACCCACGGTGCTCTGCTGTCCGGCCAGCGTGAGGCT |
| | GCCCGCCTCATTGAGATGTACCGAGACCTCTTCCAGCAGGGGACCCATCATCACCACC |
| | ATCACTGA |
| | ORF Start: at 2 ORF Stop: TGA at 1688 |
| | SEQ ID NO: 178 562 aa MW at 62742.6kD |
| NOV13f | TMQSCESSGDSADDPLSRGLRRRGQPRVVVIGAGLAGLAAAKALLEQGFTDVTVLEAS |
| 258329988 | |
| Protein Sequence | SHIGGRVQSVKLGHATFELGATWIHGSHGNPIYHLAEANGLLEETTDGERSVGRISLY |
| | SKNGVACYLTNHGRRIPKDVVEEFSDLYNEVYNLTQEFFRHDKPVNAESQNSVGVFTR |
| | EEVRNRIRNDPDDPEATKRLKLAMIQQYLKVESCESSSNSMDEVSLSAFGEWTEIPGA |
| | HHIIPSGFMRVVELLAEGIPAHVIQLGKPVRCIHWDQASARPRGPEIEPRGEGDHNHD |
| | TGEGGQGGEEPRGGRWDEDEQWSVVVECEDCELIPADHVIVTVSLGVLKRQYTSFFRP |
| | GLPTEKVAAIHRLGTGTTDKIFLEFEEPFWGPECNSLQFVWEDEAESHTLTYPPELWY |
| | RKICGFDVLYPPERYGHVLSGWICGEEALVMEKCDDEAVAEICTEMLRQFTGNPNI PK |
| | PRRILRSAWGSNPYFRGSYSYTQVGSSGADVEKLAKPLPYTESSKTAPMQVLFSGEAT |
| | HRKYYSTTHGALLSGQREAARLIEMYRDLFQQGTHHHHHH |
| | SEQ ID NO: 179 1700 bp |
| NOV13g, AAGGAAAAAAGCGGCCGCCACCATGCAAAGTTGTGAATCCAGTGGTGACAGTGCGGAT | |
| 254047897 | |
| DNA Sequence | GACCCTCTCAGTCGCGGCCTACGGAGAAGGGGACAGCCTCGTGTGGTGGTGATCGGCG |
| | CCGGCTTGGCTGGCCTGGCTGCAGCCAAAGCACTTCTTGAGCAGGGTTTCACGGATGT |
| | CACTGTGCTTGAGGCTTCCAGCCACATCGGAGGCCGTGTGCAGAGTGTGAAACTTGGA |
| | CACGCCACCTTTGAGCTGGGAGCCACCTGGATCCATGGCTCCCATGGGAACCCTATCT |
| | ATCATCTAGCAGAAGCCAACGGCCTCCTGGAAGAGACAACCGATGGGGAACGCAGCGT |
| | GGGCCGCATCAGCCTCTATTCCAAGAATGGCGTGGCCTGCTACCTTACCAACCACGGC |
| | CGCAGGATCCCCAAGGACGTGGTTGAGGAATTCAGCGATTTATACAACGAGGTCTATA |
| | ACTTGACCCAGGAGTTCTTCCGGCACGATAAACCAGTCAATGCTGAAAGTCAAAATAG |
| | CGTGGGGGTGTTCACCCGAGAGGAGGTGCGTAACCGCATCAGGAATGACCCTGACGAC |
| | CCAGAGGCTACCAAGCGCCTGAAGCTCGCCATGATCCAGCAGTACCTGAAGGTGGAGA |
| | GCTGTGAGAGCAGCTCACACAGCATGGACGAGGTGTCCCTGAGCGCCTTCGGGGAGTG |
| | GACCGAGATCCCCGGCGCTCACCACATCATCCCCTCGGGCTTCATGCGGGTTGTGGAG |
| | CTGCTGGCGGAGGGCATCCCTGCCCACGTCATCCAGCTAGGGAAACCTGTCCGCTGCA |
| | TTCACTGGGACCAGGCCTCAGCCCGCCCCAGAGGCCCTGAGATTGAGCCCCGGGGTGA |
| | GGGCGACCACAATCACGACACTGGGGAGGGTGGCCAGGGTGGAGAGGAGCCCCGGGGG |
| | GGCAGGTGGGATGAGGATGAGCAGTGGTCGGTGGTGGTGGAGTGCGAGGACTGTGAGC |
| | TGATCCCGGCGGACCATGTGATTGTGACCGTGTCGCTAGGTGTGCTAAAGAGGCAGTA |
| | CACCAGTTTCTTCCGGCCAGGCCTGCCCACAGAGAAGGTGGCTGCCATCCACCGCCTG |
| | GGCATTGGCACCACCGACAAGATCTTTCTGGAATTCGAGGAGCCCTTCTGGGGCCCTG |
| | AGTGCAACAGCCTACAGTTTGTGTGGGAGGACGAAGCAGAGAGCCACACCCTCACCTA |
| | CCCACCTGAGCTCTGGTACCGCAAGATCTGCGGCTTTGATGTCCTCTACCCGCCTGAG |
| | CGCTACGGCCATGTGCTGAGCGGCTGGATCTGCGGGGAGGAGGCCCTCGTCATGGAGA |
| | AGTGTGATGACGAGGCAGTGGCCGAGATCTGCACGGAGATGCTGCGTCAGTTCACAGG |
| | GAACCCCAACATTCCAAAACCTCGGCGAATCTTGCGCTCGGCCTGGGGCAGCAACCCT |
| | TACTTCCGCGGCTCCTATTCATACACGCAGGTGGGCTCCAGCGGGGCGGATGTGGAGA |
| | AGCTGGCCAAGCCCCTGCCGTACACGGAGAGCTCAAAGACAGCGCCCATGCAGGTGCT |
| | GTTTTCCGGTGAGGCCACCCACCGCAAGTACTATTCCACCACCCACGGTGCTCTGCTG |
| | TCCGGCCAGCGTGAGGCTGCCCGCCTCATTGAGATGTACCGAGACCTCTTCCAGCAGG |
| | GGACCTGATCTAGACTAG |
| | ORF Start: at 2 ORF Stop: TGA at 1688 |
| | SEQ ID NO: 180 562 aa MW at 62545.5kD |
| NOV13g, | RKKAAATMQSCESSGDSADDPLSRGLRRRGQPRVVVIGAGLAGTJAAAKALLEQGFTDV |
| 254047897 | |
| Protein Sequence | TVLEASSHIGGRVQSVKLGHATFELGATWIIIGSHGMPIYHLAEANGLLEETTDGERSV |
| | GRISLYSKNGVACYLTNHGRRIPKDVVEEFSDLYNEVYNLTQEFFRHDKPVNAESQNS |
| | VGVFTREEVRNRIRNDPDDPEATKRLKLANIQQYLKVESCESSSHSMDEVSLSAFGEW |
| | TEIPGANHIIPSGFMRVVELLAEGIPAHVIQLGKPVRCIHWDQASARPRGPEIEPRGE |
| | GDHNHDTGEGCQGGEEPRGGRWDEDEQWSVVVECEDCELIPADHVIVTVSLGVLKRQY |
| | TSFFRPGLPTEKVAAIHRLGIGTTDKIFLEFEEPFWGPECNSLQFVWEDEAESHTLTY |
| | PPELWYRKICGFDVLYPPERYGHVLSGWICGEEALVMEKCDDEAVAEICTEMLRQFTG |
| | NPNIPKPRRILRSAWGSNPYFRGSYSYTQVGSSGADVEKLAKPLPYTESSKTAPMQVL |
| | FSGEATHRKYYSTTHGALLSGQREAARLIEMYRDLFQQGT |
| | SEQ ID NO: 181 1690 bp |
| NOV13h, | CACCATGCAAAGTTGTGAATCCAGTGGTGACAGTGCGGATGACCCTCTCAGTCGCGGC |
| 258329988 | |
| DNA Sequence | CTACGGAGAAGGGGACAGCCTCGTGTGGTGGTGATCGGCGCCGGCTTGCCTGGCCTGG |
| | CTGCAGCCAAAGCACTTCTTGAGCAGGGTTTCACGGATGTCACTGTGCTTGAGGCTTC |
| | CAGCCACATCGGAGGCCGTGTGCAGAGTGTGAAACTTGGACACGCCACCTTTGAGCTG |
| | GGAGCCACCTGGATCCATGGCTCCCATGGGAACCCTATCTATCATCTAGCAGAAGCCA |
| | ACGGCCTCCTGGAAGAGACAACCGATGGGGAACGCAGCGTGGGCCGCATCAGCCTCTA |
| | TTCCAAGAATGGCGTGGCCTGCTACCTTACCAACCACGGCCGCAGGATCCCCAAGGAC |
| | GTGGTTGAGGAATTCAGCGATTTATACAACGAGGTCTATAACTTGACCCAGGAGTTCT |
| | TCCGGCACGATAAACCAGTCAATGCTGAAAGTCAAAATAGCGTGGGGGTGTTCACCCG |
| | AGAGGAGGTGCGTAACCGCATCAGGAATGACCCTGACGACCCAGAGGCTACCAAGCGC |
| | CTGAAGCTCGCCATGATCCAGCAGTACCTGAAGGTGGAGAGCTGTGAGAGCAGCTCAC |
| | ACAGCATGGACGAGGTGTCCCTGAGCGCCTTCGGGGAGTGGACCGAGATCCCCGGCGC |
| | TCACCACATCATCCCCTCGGGCTTCATGCGGGTTGTGGAGCTGCTGGCGGAGGGCATC |
| | CCTGCCCACGTCATCCAGCTAGGGAAACCTGTCCGCTGCATTCACTGGGACCAGGCCT |
| | CAGCCCGCCCCAGAGGCCCTGAGATTGAGCCCCGGGGTGAGGGCGACCACAATCACGA |
| | CACTGGGGAGGGTGGCCAGGGTGGAGAGGAGCCCCGGGGGGGCACGTGGGATGAGGAT |
| | GAGCAGTGGTCGGTGGTGGTGGAGTGCGAGGACTGTGAGCTGATCCCGGCGGACCATG |
| | TGATTGTGACCGTGTCGCTAGGTGTGCTAAAGAGGCAGTACACCAGTTTCTTCCGGCC |
| | AGGCCTGCCCACAGAGAAGGTGGCTGCCATCCACCGCCTGGGCATTGGCACCACCGAC |
| | AAGATCTTTCTGGAATTCGAGGAGCCCTTCTGGGGCCCTGAGTGCAACAGCCTACAGT |
| | TTGTGTGGGAGGACGAAGCAGAGAGCCACACCCTCACCTACCCACCTGAGCTCTGGTA |
| | CCCCAAGATCTGCGCCTTTGATGTCCTCTACCCGCCTGAGCGCTACGGCCATGTGCTG |
| | AGCGGCTGGATCTGCGGGGAGGAGGCCCTCGTCATGGAGAAGTGTGATGACGAGGCAG |
| | TGGCCGAGATCTGCACGGAGATGCTGCGTCAGTTCACAGGGAACCCCAACATTCCAAA |
| | ACCTCGGCGAATCTTGCGCTCGGCCTGGGGCAGCAACCCTTACTTCCGCGGCTCCTAT |
| | TCATACACGCAGGTGGGCTCCAGCGGGGCGGATGTGGAGAAGCTGGCCAAGCCCCTGC |
| | CGTACACCGAGAGCTCAAAGACAGCGCCCATGCAGGTGCTGTTTTCCGGTGAGGCCAC |
| | CCACCGCAAGTACTATTCCACCACCCACGGTGCTCTGCTGTCCGGCCAGCGTGAGGCT |
| | GCCCGCCTCATTGAGATGTACCGAGACCTCTTCCAGCAGGGGACCCATCATCACCACC |
| | ATCACTGA |
| | ORF Start: at 2 ORF Stop: TGA at 1688 |
| | SEQ ID NO: 182 562 aa MW at 62742.6kD |
| NOV13h, | TMQSCESSGDSADDPLSRGLRRRGQPRVVVIGAGLAGLAAAKALLEQGFTDVTVLEAS |
| 258329988 | |
| Protein Sequence | SHIGGRVQSVKLGHATFELGATWIHGSHGNPIYHLAEANGLLEETTDGERSVGRISLY |
| | SKNGVACYLTNHGRRIPKDVVEEFSDLYNEVYNLTQEFFRHDKPVNAESQNSVGVFTR |
| | EEVRNRIRNDPDDPEATKRLKLANIQQYLKVESCESSSHSMDEVSLSAFGEWTEIPGA |
| | HHIIPSGFMRVVELLAEGIPAHVIQLGKPVRCIHWDQASARPRGPEIEPRGEGDHNHD |
| | TGEGGQGGEEPRGGRWDEDEQWSVVVECEDCELIPADHVIVTVSLGVLKRQYTSFFRP |
| | GLPTEKVAAIHRLGIGTTDKIFLEFEEPFWGPECNSLQFVWEDEAESHTLTYPPELWY |
| | RKICGFDVLYPPERYGHVLSGWICGEEALVMEKCDDEAVAEICTEMLRQPTGNPNIPK |
| | PRRILRSAWGSNPYFRGSYSYTQVGSSGADVEKLAKPLPYTESSKTAPMQVLFSGEAT |
| | HRKYYSTTHGALLSGQREAARLIEMYRDLFQQGTHHHHHH |
| | SEQ ID NO: 183 1672 bp |
| NOV13j, | CACCATGCAAAGTTGTGAATCCAGTGGTGACAGTGCGGATGACCCTCTCAGTCGCGGC |
| 258280066 | |
| DNA Sequence | CTACGGAGAAGGGGACAGCCTCGTGTGGTGGTGATCGGCGCCGGCTTGGCTGGCCTGG |
| | CTGCAGCCAAAGCACTTCTTGAGCAGGGTTTCACGGATGTCACTGTGCTTGAGCCTTC |
| | CAGCCACATCGGAGGCCGTGTGCAGAGTGTGAAACTTGGACACGCCACCTTTGAGCTG |
| | GGAGCCACCTGGATCCATGGCTCCCATGGGAACCCTATCTATCATCTAGCAGAAGCCA |
| | ACGGCCTCCTGGAAGAGACAACCGATGGGGAACGCAGCGTGGGCCGCATCAGCCTCTA |
| | TTCCAAGAATGGCGTGGCCTGCTACCTTACCAACCACGGCCGCAGGATCCCCAAGGAC |
| | GTGGTTGAGGAATTCAGCGATTTATACAACGAGGTCTATAACTTGACCCAGGAGTTCT |
| | TCCGGCACGATAAACCAGTCAATGCTGAAAGTCAAAATAGCGTGGGGGTGTTCACCCG |
| | AGAGGAGGTGCGTAACCGCATCAGGAATGACCCTGACGACCCAGAGGCTACCAAGCGC |
| | CTGAAGCTCGCCATGATCCAGCAGTACCTGAAGGTGGAGAGCTGTGAGAGCAGCTCAC |
| | ACAGCATGGACGAGGTGTCCCTGAGCGCCTTCGGGGAGTGGACCGAGATCCCCGGCGC |
| | TCACCACATCATCCCCTCGCGCTTCATGCGGGTTGTGGAGCTGCTGGCGGAGGGCATC |
| | CCTGCCCACGTCATCCAGCTAGGGAAACCTGTCCGCTGCATTCACTGGGACCAGGCCT |
| | CAGCCCGCCCCAGAGGCCCTGAGATTGAGCCCCGGGGTGAGGGCGACCACAATCACGA |
| | CACTGCGGAGGGTGGCCAGGGTGGAGAGGAGCCCCGGGGGGGCAGGTGGGATGAGGAT |
| | GAGCAGTGGTCGGTGGTGGTGGAGTGCGAGGACTGTGAGCTGATCCCGGCGGACCATG |
| | TGATTGTGACCGTGTCGCTAGGTGTGCTAAAGAGGCAGTACACCAGTTTCTTCCGGCC |
| | AGGCCTGCCCACAGAGAAGGTGGCTGCCATCCACCGCCTGGGCATTGGCACCACCGAC |
| | AAGATCTTTCTGGAATTCGAGGAGCCCTTCTGGGGCCCTGAGTGCAACAGCCTACAGT |
| | TTGTGTGGGAGGACGAAGCAGAGAGCCACACCCTCACCTACCCACCTGAGCTCTGGTA |
| | CCGCAAGATCTGCGGCTTTGATGTCCTCTACCCGCCTGAGCGCTACGGCCATGTGCTG |
| | AGCGGCTGGATCTGCGGGGAGGAGGCCCTCGTCATGGAGAAGTGTGATGACGAGGCAG |
| | TGGCCGAGATCTGCACGGAGATGCTGCGTCAGTTCACAGGGAACCCCAACATTCCAAA |
| | ACCTCGGCGAATCTTGCGCTCGGCCTGGGGCAGCAACCCTTACTTCCGCGGCTCCTAT |
| | TCATACACGCAGGTGGGCTCCAGCGGGGCGGATGTGGAGAAGCTGGCCAAGCCCCTGC |
| | CGTACACGGAGAGCTCAAAGACAGCGCCCATGCAGGTGCTGTTTTCCGGTGAGGCCAC |
| | CCACCGCAAGTACTATTCCACCACCCACGGTGCTCTGCTGTCCGGCCAGCGTGAGGCT |
| | GCCCGCCTCATTGAGATGTACCGAGACCTCTTCCAGCAGGGGACCTGA |
| | ORF Start: at 2 ORF Stop: TGA at 1670 |
| | SEQ ID NO: 184 556 aa MW at 61919.7kD |
| NOV13j, | TMQSCESSGDSADDPLSRGLRRRGQPRVVVIGAGLAGLAAAKALLEQGFTDVTVLEAS |
| 258280066 | |
| Protein Sequence | SHIGGRVQSVKLGHATFELGATWIHGSHGNPIYHLAEANGLLEETTDGERSVGRISLY |
| | SKNGVACYLTNHGRRIPKDVVEEFSDLYNEVYNLTQEFFRHDKPVNAESQNSVGVFTR |
| | EEVRNRIRNDPDDPEATKRLKLAMIQQYLKVESCESSSHSMDEVSLSAFGEWTEIPGA |
| | HHIIPSGFMRVVELLAEGIPAHVIQLGKPVRCIHWDQASARPRGPEIEPRGEGDHNHD |
| | TGEGGQGGEEPRGGRWDEDEQWSVVVECEDCELIPADHVIVTVSLGVLKRQYTSFFRP |
| | GLPTEKVAAIHRLGIGTTDKIFLEFEEPFWGPECNSLQFVWEDEAESHTLTYPPELWY |
| | RKICGFDVLYPPERYGHVLSGWICGEEALVMEKCDDEAVAEICTEMLRQFTGNPNIPK |
| | PRRILRSAWGSNPYFRGSYSYTQVGSSGADVEKLAKPLPYTESSKTAPMQVLFSGEAT |
| | HRKYYSTTHGALLSGQREAARLIEMYRDLFQQGT |
| | SEQ ID NO: 185 1693 bp |
| NOV13j, | CACCATGGGACATCATCACCACCATCACCAAAGTTGTGAATCCAGTGGTGACAGTGCG |
| 258280083 | |
| DNA Sequence | GATGACCCTCTCAGTCGCGGCCTACGGAGAAGGGGACAGCCTCGTGTGGTGGTGATCG |
| | GCGCCGGCTTGGCTGGCCTGGCTGCAGCCAAAGCACTTCTTGAGCAGGGTTTCACGGA |
| | TGTCACTGTCCTTGAGGCTTCCAGCCACATCGGAGGCCGTGTGCAGAGTGTGAAACTT |
| | GGACACGCCACCTTTGAGCTGGGAGCCACCTGGATCCATGGCTCCCATGGCAACCCTA |
| | TCTATCATCTAGCAGAAGCCAACGGCCTCCTGGAAGAGACAACCGATGGGGAACGCAG |
| | CGTGGGCCGCATCAGCCTCTATTCCAAGAATGGCGTGGCCTGCTACCTTACCAACCAC |
| | GGCCGCAGGATCCCCAAGGACGTGGTTGAGGAATTCAGCGATTTATACAACGACGTCT |
| | ATAACTTGACCCAGGAGTTCTTCCGGCACGATAAACCAGTCAATGCTGAAAGTCAAAA |
| | TAGCGTGGGGGTGTTCACCCGAGACGAGGTGCGTAACCGCATCAGGAATGACCCTGAC |
| | GACCCAGAGGCTACCAAGCGCCTGAACCTCGCCATGATCCAGCAGTACCTGAAGGTGG |
| | AGAGCTGTGAGAGCAGCTCACACAGCATGGACGAGGTGTCCCTGAGCGCCTTCGGGGA |
| | GTGGACCGAGATCCCCGGCGCTCACCACATCATCCCCTCGGGCTTCATGCGGGTTGTG |
| | GAGCTGCTGGCGGAGGGCATCCCTCCCCACGTCATCCAGCTAGGGAAACCTGTCCGCT |
| | GCATTCACTGGGACCAGGCCTCAGCCCGCCCCAGAGGCCCTGAGATTGAGCCCCGGGG |
| | TGAGGGCGACCACAATCACGACACTGGGGAGGGTGGCCAGGGTGGAGAGGAGCCCCGG |
| | GGCGGCAGGTGGGATGAGGATGAGCAGTGGTCGGTCGTGGTGGAGTGCGAGGACTGTG |
| | AGCTGATCCCGGCGGACCATGTGATTGTGACCGTGTCGCTAGGTGTGCTAAAGAGGCA |
| | GTACACCAGTTTCTTCCGGCCAGGCCTGCCCACAGAGAAGGTGGCTGCCATCCACCGC |
| | CTGGGCATTGGCACCACCGACAAGATCTTTCTGGAATTCGAGGAGCCCTTCTGGGGCC |
| | CTGAGTGCAACAGCCTACAGTTTGTGTGGGAGGACGAAGCAGAGAGCCACACCCTCAC |
| | CTACCCACCTGAGCTCTGGTACCGCAAGATCTGCGGCTTTGATGTCCTCTACCCGCCT |
| | GAGCGCTACGGCCATGTGCTGAGCGGCTGGATCTGCGGGGAGGAGGCCCTCGTCATGG |
| | AGAAGTGTGATGACGAGGCAGTGGCCGAGATCTGCACGGAGATGCTGCGTCAGTTCAC |
| | AGGGAACCCCAACATTCCAAAACCTCGGCGAATCTTGCGCTCGGCCTGGGGCACCAAC |
| | CCTTACTTCCGCGGCTCCTATTCATACACGCAGGTGGGCTCCAGCGGGGCGGATGTGG |
| | AGAAGCTGGCCAAGCCCCTGCCGTACACGGAGAGCTCAAAGACAGCGCCCATGCAGGT |
| | GCTGTTTTCCGGTGAGGCCACCCACCGCAAGTACTATTCCACCACCCACGGTGCTCTG |
| | CTGTCCGGCCAGCGTGAGGCTGCCCGCCTCATTCAGATGTAcCGAGACCTCTTCCAGC |
| | AGGGGACCTGA |
| | ORF Start: at 2 ORF Stop: TGA at 1691 |
| | SEQ ID NO: 186 563 aa MW at 62799.6kD |
| NOV13j, | TMGHHHHHHQSCESSGDSADDPLSRGLRRRGQPRVVVIGAGLAGLAAAKALLEQGFTD |
| 258280083 | |
| Protein Sequence | VTVLEASSHIGGRVQSVKLGHATFELGATWIHGSHGNPIYHLAEANGLLEETTDGERS |
| | VGRISLYSKNGVACYLTNHGRRIPKDVVEEFSDLYNEVYNLTQEFFRHDKPVNAESQN |
| | SVGVFTREEVRNRIRNDPDDPEATKRLKLAMIQQYLKVESCESSSHSMDEVSLSAFGE |
| | WTEIPGAHHIISGFMRVVELLAEGIPAHVIQLGKPVRCIHWDQASARPRGPEIEPRG |
| | EGDHNHDTGEGGQGGEEPRGGRWDEDEQWSVVVECEDCELIPADHVIVTVSLGVLKRQ |
| | YTSFFRPGLPTEKVAAIHRLGIGTTDKIFLEFEEPFWGPECNSLQFVWEDEAESHTLT |
| | YPPELWYRKICGFDVLYPPERYGHVLSGWICGEEALVMEKCDDEAVAEICTEMLRQFT |
| | GNPNIPKPRRILRSAWGSNPYFRGSYSYTQVGSSGADVEKLAKPLPYTESSKTAPMQV |
| | LFSGEATHRKYYSTTHGALLSGQREAARLIEMYRDLFQQGT |
| | SEQ ID NO: 187 1993 bp |
| NOV13k | GGCACGAGGGTCCCGGCGGCGGCTGGAGGAGGAAGCCAGGCGGCTGGCGGAGGAGGAG |
| CG140122-02 | |
| DNA Sequence | AGACGGAGGAGGCCGAGACCGGAGCGCCGCTCGCCGCAGACTTACTTCCCCGGCTCAG |
| | CAGGGAAAGGTTCCTAGAAGGTGAGCGCGGACGGTATGCAAAGTTGTGAATCCAGTGG |
| | TGACAGTGCGGATGACCCTCTCAGTCGCGGCCTACGGAGAAGCGGACAGCCTCGTGTG |
| | GTGGTGATCGGCGCCGGCTTGGCTGGCCTGGCTGCAGCCAAAGCACTTCTTGACCAGG |
| | GTTTCACGGATGTCACTGTCCTTGAGGCTTCCAGCCACATCGGAGGCCGTGTGCAGAG |
| | TGTGAAACTTGGACACGCCACCTTTGACCTGGGAGCCACCTGGATCCATGGCTCCCAT |
| | GGGAACCCTATCTATCATCTAGCAGAGCCAACGGCCTCCTGGAAGAGACAACCGATG |
| | GGGAACGCAGCGTGGGCCGCATCAGCCTCTATTCCAAGAATGGCGTGGCCTGCTACCT |
| | TACCAACCACGGCCGCAGCATCCCCAAGGACGTGGTTGAGGAATTCAGCGATTTATAC |
| | AACGAGGTCTATAACTTGACCCAGGAGTTCTTCCGGCACGATAAACCAGTCAATGCTG |
| | AAAGTCAAAATAGCGTGGGGGTGTTCACCCGAGAGGAGGTGCGTAACCGCATCAGGAA |
| | TGACCCTGACGACCCAGAGGCTACCAAGCGCCTGAAGCTCGCCATGATCCAGCAGTAc |
| | CTGAAGGTGGAGAGCTGTGAGAGCAGCTCACACAGCATGGACGAGGTGTCCCTGAGCG |
| | CCTTCGGGGAGTGGACCGAGATCCCCGGCGCTCACCACATCATCCCCTCGGGCTTCAT |
| | GCGGGTTGTGGAGCTGCTGGCGGAGGGCATCCCTGCCCACGTCATCCAGCTAGGGAAA |
| | CCTGTCCGCTGCATTCACTGGGACCAGGCCTCAGCCCGCCCCAGAGGCCCTGAGATTG |
| | AGCCCCGGGGTGTGCTAAAGAGGCAGTACACCAGTTTCTTCCGGCCAGGCCTGCCCAC |
| | AGAGAAGGTGGCTGCCATCCACCGCCTGGGCATTGGCACCACCGACAAGATCTTTCTG |
| | GAATTCGAGGAGCCCTTCTGGGGCCCTGAGTGCAACAGCCTACAGTTTGTGTGGGAGG |
| | ACGAAGCGGAGAGCCACACCCTCACCTACCCACCTGAGCTCTGGTACCGCAAGATCTG |
| | CGGCTTTGATGTCCTCTACCCGCCTGAGCGCTACGGCCATGTGCTGAGCGGCTGGATC |
| | TGCGGGGAGGAGGCCCTCGTCATGGAGAAGTGTGATGACGAGGCAGTGGCCGAGATCT |
| | GCACGGAGATGCTGCGTCAGTTCACAGGGAACCCCAACATTCCAAAACCTCGGCGAAT |
| | CTTGCGCTCGGCCTGGGGCAGCAACCCTTACTTCCGCGGCTCCTATTCATACACGCAG |
| | GTGGGCTCCAGCGGGGCGGATGTGGAGAAGCTGGCCAAGCCCCTGCCGTACACGGAGA |
| | GCTCAAAGACAGCGCCCATGCAGGTGCTGTTTTCCGGTGAGGCCACCCACCGCAAGTA |
| | CTATTCCACCACCCACGGTGCTCTGCTGTCCGGCCAGCGTGAGGCTGCCCGCCTCATT |
| | GAGATGTACCGAGACCTCTTCCAGCAGGGGACCTGAGGGCTGTCCTCGCTGCTGAGAA |
| | UGAGCCACTAACTCGTGACCTCCAGCCTGCCCCTTGCTGCCGTGTGCTCCTGCCTTCCT |
| | GATCCTCTGTAGAAAGGATTTTTATCTTCTGTAGAGCTAGCCGCCCTGACTGCCTTCA |
| | GACCTGGCCCTGTAGCTTTTCTTTTTCTCCAGGCTGGGCCGTGAGCAGGTGGGCCGTT |
| | GAGTTACCTCTGTGCTGGATCCCGTGCCCCCACTTGCCTACCCTCTGTCCTCCCTTGT |
| | TATTGTAAGTGCCTTCAATACTTTGCATTTTGGGATAATAAAAAAGGCTCCCTCCCCT |
| | GCAAAAAAAAAAAAAAAAAAA |
| | ORF Start: ATG at 152 ORF Stop: TGA at 1658 |
| | SEQ ID NO: 188 502 aa MW at 56090.6kD |
| NOV13k, | MQSCESSGDSADDPLSRGLRRRGQPRVVVIGAGLAGLAAAKALLEQGFTDVTVLEASS |
| CG140122-02 | |
| Protein Sequence | HIGGRVQSVKLGHATFELGATWIHGSHGNPIYHLAEANGLLEETTDGERSVGRISLYS |
| | KNGVACYLTNHGRRIPKDVVEEFSDLYNEVYNLTQEFFRHDKPVNAESQNSVGVFTRE |
| | EVRNRIRNDPDDPEATKRLKLAMIQQYLKVESCESSSHSMDEVSLSAFGEWTEIPGAH |
| | HIIPSGFMRVVELLAEGIPAHVIQLCKPVRCIHWDQASARPRGPEIEPRGVLKRQYTS |
| | FFRPGLPTEKVAAIHRLGIGTTDKIFLEFEEPFWGPECNSLQFVWEDEAESHTLTYPP |
| | ELWYRKICGFDVLYPPERYGHVLSGWICGEEALVMEKCDDEAVAEICTEMLRQFTGNP |
| | NIPKPRRILRSAWGSNPYFRGSYSYTQVGSSGADVEKLAKPLPYTESSKTAPMQVLFS |
| | CEATHRKYYSTTHGALLSGQREAARLIEMYRDLFQQGT |
| | SEQ ID NO: 189 1012 bp |
| NOV13l, | CACCATGCAAAGTTGTGAATCCAGTGGTGACAGTGCGGATGACCCTCTCAGTCGCGGC |
| CG140122-03 | |
| DNA Sequence | CTACGGAGAAGGGCACAGCCTCGTGTGGTGGTGATCGGCGCCGGCTTGGCTGGCCTGG |
| | CTGCAGCCAAAGCACTTCTTGAGCAGGGTTTCACGGATGTCACTGTGCTTGAGGCTTC |
| | CAGCCACATCGGAGGCCGTGTGCAGAGTGTGAAACTTGGACACGCCACCTTTGAGCTG |
| | GGAGCCACCTGGATCCATGGCTCCCATGGGAACCCTATCTATCATCTAGCAGAAGCCA |
| | ACGGCCTCCTGCAAGAGACAACCGATCGGGAACGCAGCGTGGGCCGCATCAGCCTCTA |
| | TTCCAAGAATGGCGTGGCCTGCTACCTTACCAACCACCGCCGCAGCATCCCCAAGGAC |
| | GTGGTTGAGGAATTCAGCGATTTATACAACGAGGTCTATAACTTGACCCAGGAGTTCT |
| | TCCGGCACGATAAACCAGTCAATGCTGAAAGTCAAAATAGCGTGGGGGTGTTCACCCG |
| | AGAGGAGGTGCGTAACCGCATCAGGAATGACCCTGACGACCCAGAGGCTACCAAGCGC |
| | CTGAAGCTCGCCATGATCCAGCAGTACCTGAAGGTGGAGAGCTGTGAGAGCAGCTCAC |
| | ACAGCATGGACGAGGTGTCCCTGAGCGCCTTCGGGGAGTGGACCGAGATCCCCGGcGC |
| | TCACCACATCATCCCCTCGGGCTTCATGCGGGTTGTGGAGCTGCTGGCGGAGGGCATC |
| | CCTGCCCACGTCATCCAGCTAGGGAAACCTGTCCGCTGCATTCACTGGGACCAGGCCT |
| | CAGCCCGCCCCAGAGGCCCTGAGATTGAGCCCCTGCCGTACACAGAGAGCTCAAAGAC |
| | AGCGCCCATGCAGGTGCTGTTTTCCGGTGAGCCCACCCACCGCAAGTACTATTCCACC |
| | ACCCACGGTGCTCTCCTGTCCGGCCAGCGTGAGGCTGCCCGCCTCATTGAGATGTACC |
| | GACACCTCTTCCAGCAGGGGACCTGA |
| | ORF Start: at 2 ORF Stop: TGA at 1010 |
| | SEQ ID NO: 190 336 aa MW at 37093.2kD |
| NOV13L, | TMQSCESSGDSADDPLSRGLRRRGQPRVVVIGAGLAGLAAAKALLEQGFTDVTVLEAS |
| CG140122-03 | |
| Protein Sequence | SHIGGRVQSVKLGHATFELGATWIHGSHGNPIYHLAEANGLLEETTDGERSVGRISLY |
| | SKNGVACYLTNHGRRIPKDVVEEFSDLYNEVYNLTQEFFRHDKPVNAESQNSVGVFTR |
| | EEVRNRIRNDPDDPEATKRLKLAMIQQYLKVESCESSSHSMDEVSLSAFGEWTEIPGA |
| | RHIIPSGFMRVVELLAEGIPAHXTIQLGKPVRCIHWDQASARPRGPEIEPLPYTESSKT |
| | APMQVLFSGEATHRKYYSTTHGALLSGQREAARLIEMYRDLFQQGT |
| | SEQ ID NO: 191 1603 bp |
| NOV13m, | CACCATGCAAAGTTGTGAATCCAGTGGTGACAGTGCGGATGACCCTCTCAGTCGCGGC |
| CG140122-04 | |
| DNA Sequence | CTACGGAGAAGGGGACAGCCTCGTGTGGTGGTGATCGGCGCCGGCTTGGCTGGCCTGG |
| | CTGCAGCCAAAGCACTTCTTGAGCAGGGTTTCACGGATGTCACTGTGCTTGAGGCTTC |
| | CAGCCACATCGGAGGCCGTGTGCAGAGTGTGAAACTTGGACACGCCACCTTTGAGCTG |
| | GGAGCCACCTGGATCCATGGCTCCCATGGGAACCCTATCTATCATCTAGCAGAAQCCA |
| | ACGGCCTCCTGGAAGAGACAACCGATGGGGAACGCAGCGTGGGCCGCATCAGCCTCTA |
| | TTCCAAGAATGGCGTGGCCTGCTACCTTACCAACCACGGCCGCAGGATCCCCAAGGAC |
| | GTGGTTGAGGAATTCAGCGATTTATACAACGAGGTCTATAACTTGACCCAGGAGTTCT |
| | TCCGGCACGATAAACCAGTCAATGCTGAAAGTCAAAATAGCGTGGGGGTGTTCACCCG |
| | AGAGGAGGTGCGTAACCGCATCAGGAATGACCCTGACGACCCAGAGGCTACCAAGCGC |
| | CTGAAGCTCGCCATGATCCAGCAGTACCTGAAGGTGGAGAGCTGTGAGAGCAGCTCAC |
| | ACAGCATGGACGAGGTGTCCCTGAGCGCCTTCGGGGAGTGGACCGAGATCCCCGGCGC |
| | TCACCACATCATCCCCTCGGGCTTCATGCGGGTTGTGGAGCTGCTGGCGGAGGGCATC |
| | CCTGCCCACGTCATCCAGCTAGGGAAACCTGTCCGCTGCATTCACTGGGACCAGGCCT |
| | CAGCCCGCCCCAGAGGCCCTGAGATTGAGCCCCGGGGTGTGCTAAAGAGGCAGTACAC |
| | CAGTTTCTTCCGGCCAGGCCTGCCCACAGAGAAGGTGGCTGCCATCCACCGCCTGGGC |
| | ATTGGCACCACCGACAAGATCTTTCTGGAATTCGAGGAGCCCTTCTGGGGCCCTGAGT |
| | GCAACAGCCTACAGTTTGTGTGGGAGGACGAAGCGGAGAGCCACACCCTCACCTACCC |
| | ACCTGAGCTCTGGTACCGCAAGATCTGCGGCTTTGATGTCCTCTACCCGCCTGAGCGC |
| | TACGGCCATGTGCTGAGCGGCTGGATCTGCGGGGAGGAGGCCCTCGTCATGGAGAAGT |
| | GTGATGACGAGGCAGTGGCCGAGATCTGCACGGAGATGCTGCGTCAGTTCACAGGGAA |
| | CCCCAACATTCCAAAACCTCGGCGAATCTTGCGCTCGGCCTGGGGCAGCAACCCTTAC |
| | TTCCGCGGCTCCTATTCATACACGCAGGTGGGCTCCAGCGGGGCGGATGTGGAGAAGC |
| | TGGCCAAGCCCCTGCCGTACACGGAGAGCTCAAAGACAGCGCATGGAAGCTCCACAAA |
| | GCAGCAGCCTGGTCACCTTTTCTCTTCCAAGTGCCCAGAACAGCCCCTGGATGCTAAC |
| | AGGGGCGCCGTAAAGCCCATGCAGGTGCTGTTTTCCGGTGAGGCCACCCACCGCAAGT |
| | ACTATTCCACCACCCACGGTGCTCTGCTGTCCGGCCAGCGTGAGGCTGCCCGCCTCAT |
| | TGAGATGTACCGAGACCTCTTCCAGCAGGGGACCTGA |
| | ORF Start: at 2 ORF Stop: TGA at 1601 |
| | SEQ ID NO: 192 533 aa MW at 59379.2kD |
| NOV13m, | TMQSCESSGDSADDPLSRGLRRRGQPRVVVIGAGLAGLAAAAKALLEQGFTDVTVLEAS |
| CG140122-04 | |
| Protein Sequence | SHIGGRVQSVKLGHATFELGATWIHGSHGNPIYHLAEANGLLEETTDGERSVGRISLY |
| | SKNGVACYLTNHGRRIPKDVVEEFSDLYNEVYNLTQEFFRHDKPVNAESQNSVGVFTR |
| | EEVRNRIRNDPDDPEATKRLKLAMIQQYLKVESCESSSHSMDEVSLSAFGEWTEIPGA |
| | HHIIPSGFMRVVELLAEGIPAHVIQLGKPVRCIHWDQASARPRGPEIEPRGVLKRQYT |
| | SFFRPGLPTEKVAAIHRLGIGTTDKIFLEFEEPFWGPECNSLQFVWEDEAESHTLTYP |
| | PELWYRKICGFDVLYPPERYGHVLSGWICGEEALVMEKCDDEAVAEICTEMLRQFTGN |
| | PNIPKPRRILRSAWGSNPYFRGSYSYTQVGSSGADVEKLAKPLPYTESSKTAHGSSTK |
| | QQPGHLFSSKCPEQPLDANRGAVKPMQVLFSGEATHRKYYSTTHGALLSGQREAARLT |
| | EMYRDLFQQGT |
| | SEQ ID NO: 193 1513 bp |
| NOV13n, | CACCATGCAAAGTTGTGAATCCAGTGGTGACAGTGCGGATGACCCTCTCAGTCGCGGC |
| CG140122-05 | |
| DNA Sequence | CTACGGAGAAGGGGACAGCCTCGTGTGGTGGTGATCGGCGCCGGCTTGGCTGGCCTGG |
| | CTGCAGCCAAAGCACTTCTTGAGCAGGGTTTCACGGATGTCACTGTGCTTGAGGCTTC |
| | CAGCCACATCGGAGGCCGTGTGCAGAGTGTGAAACTTGGACACGCCACCTTTGAGCTG |
| | GGAGCCACCTGGATCCATGGCTCCCATGGGAACCCTATCTATCATCTAGCAGAAGCCA |
| | ACGGCCTCCTGGAAGAGACAACCGATGGGGAACGCAGCCTGGGCCGCATCAGCCTCTA |
| | TTCCAAGAATGGCGTGGCCTGCTACCTTACCAACCACGGCCGCAGGATCCCCAAGGAC |
| | GTGGTTGAGGAATTCAGCGATTTATACAACGAGGTCTATAACTTGACCCAGGAGTTCT |
| | TCCGGCACGATAAACCAGTCAATGCTGAAAGTCAAAATAGCGTGGGGGTGTTCACCCG |
| | AGAGGAGGTGCGTAACCGCATCAGGAATGACCCTGACGACCCAGAGGCTACCAAGCGC |
| | CTGAAGCTCGCCATGATCCAGCAGTACCTGAAGGTGGAGAGCTGTGAGAGCAGCTCAC |
| | ACAGCATGGACGAGGTGTCCCTGAGCGCCTTCGGGGAGTGGACCGAGATCCCCGGCGC |
| | TCACCACATCATCCCCTCGGGCTTCATGCGGGTTGTGGAGCTGCTGGCGGAGGGCATC |
| | CCTGCCCACGTCATCCAGCTAGGGAAACCTGTCCGCTGCATTCACTGGGACCAGGCCT |
| | CAGCCCGCCCCAGAGGCCCTGAGATTGAGCCCCGGGGTGTGCTAAAGAGGCAGTACAC |
| | CAGTTTCTTCCGGCCAGGCCTGCCCACAGAGAAGGTGGCTGCCATCCACCGCCTGGGC |
| | ATTGGCACCACCGACAAGATCTTTCTGGAATTCGAGGAGCCCTTCTGGGGCCCTGAGT |
| | GCAACAGCCTACAGTTTGTGTGGGAGGACGAAGCGGAGAGCCACACCCTCACCTACCC |
| | ACCTGAGCTCTGGTACCGCAAGATCTGCGGCTTTGATGTCCTCTACCCGCCTGAGCGC |
| | TACGOCCATGTGCTGAGCGGCTGGATCTGCGGGGAGGAGGCCCTCGTCATGGAGAAGT |
| | GTGATGACGAGGCAGTGGCCGAGATCTGCACGGAGATGCTGCGTCAGTTCACAGGGAA |
| | CCCCAACATTCCAAAACCTCGGCGAATCTTGCGCTCGGCCTGGGGCAGCAACCCTTAC |
| | TTCCGCGGCTCCTATTCATACACGCAGGTGGGCTCCAGCGGGGCGGATGTGGAGAAGC |
| | TGGCCAAGCCCCTGCCGTACACGGAGAGCTCAAAGACAGCGCCCATGCAGGTGCTGTT |
| | TTCCGGTGAGGCCACCCACCGCAAGTACTATTCCACCACCCACGGTGCTCTGCTGTCC |
| | GGCCAGCGTGAGGCTGCCCGCCTCATTGAGATGTACCGAGACCTCTTCCAGCAGGGGA |
| | CCTGA |
| | ORF Start: at 2 ORF Stop: TGA at 1511 |
| | SEQ ID NO: 194 503 aa MW at 56191.7kD |
| NOV13n, | TMQSCESSGDSADDPLSRGLRRRGQPRVVVIGAGLAGLAAAKALLEQGFTDVTVLEAS |
| CG14O122-05 | |
| Protein Sequence | SHIGGRVQSVKLGHATFELGATWIHGSHGNPIYHLAEANGLLEETTDGERSVGRISLY |
| | SKNGVACYLTNHGRRIPKDVVEEFSDLYNEVYNLTQEFFRHDKPVNAESQNSVGVFTR |
| | EEVRNRIRNDPDDPEATKRLKLAMIQQYLKVESCESSSHSMDEVSLSAFGEWTEIPGA |
| | HHIIPSGFMRVVELLAEGIPAHVIQLGKPVRCIHWDQASARPRGPEIEPRGVLKRQYT |
| | SFFRPGLPTEKVAAIHRLGIGTTDKIFLEFEEPFWGPECNSLQFVWEDEAESHTLTYP |
| | PELWYRKICGFDVLYPPERYGHVLSGWICGEEALVMEKCDDEAVAEICTEMLRQFTGN |
| | PNIPKPRRILRSAWGSNPYFRGSYSYTQVGSSGADVEKLAKPLPYTESSKTAPMQVLF |
| | SGEATHRKYYSTTHGALLSGQREAARLIEMYRDLFQQGT |
| | SEQ ID NO: 195 1693 bp |
| NOV13o, | CACCATGGGACATCATCACCACCATCACCAAAGTTGTGAATCCAGTGGTGACAGTGCG |
| CG140122-06 | |
| DNA Sequence | GATGACCCTCTCAGTCGCGGCCTACGGAGAAGGGGACAGCCTCGTGTGGTGGTGATCG |
| | CCGCCGGCTTGGCTGGCCTGGCTGCAGCCAAAGCACTTCTTGAGCAGGGTTTCACGGA |
| | TGTCACTGTGCTTGAGGCTTCCAGCCACATCGGAGGCCGTGTGCACAGTGTGAAACTT |
| | GGACACGCCACCTTTGAGCTGGGAGCCACCTGGATCCATGGCTCCCATGGGAACCCTA |
| | TCTATCATCTAGCAGAAGCCAACGGCCTCCTGGAAGAGACAACCGATGGGGAACGCAG |
| | CGTGGGCCGCATCAGCCTCTATTCCAAGAATGGCGTGGCCTGCTACCTTACCAACCAC |
| | GGCCGCAGGATCCCCAAGGACGTGGTTGAGGAATTCAGCGATTTATACAACGAGGTCT |
| | ATAACTTGACCCAGGAGTTCTTCCGGCACGATAAACCAGTCAATGCTGAAAGTCAAAA |
| | TAGCGTGGGGGTGTTCACCCGAGAGGAGGTGCGTAACCGCATCAGGAATGACCCTGAC |
| | GACCCAGAGGCTACCAAGCGCCTGAAGCTCGCCATGATCCAGCAGTACCTGAAGGTGG |
| | AGAGCTGTGAGAGCAGCTCACACAGCATGGACGAGGTGTCCCTGAGCGCCTTCGGGGA |
| | GTGGACCGAGATCCCCGGCGCTCACCACATCATCCCCTCGGGCTTCATGCGGGTTGTG |
| | GAGCTGCTGGCGGAGGGCATCCCTGCCCACGTCATCCAGCTAGGGAAACCTGTCCGCT |
| | GCATTCACTGGGACCAGGCCTCAGCCCGCCCCAGAGGCCCTGAGATTGAGCCCCGGGG |
| | TGAGGGCGACCACAATCACGACACTGGGGAGGGTGGCCAGGGTGGAGAGGAGCCCCGG |
| | GGGGGCAGGTGGGATGAGGATGAGCAGTGGTCGGTGGTGGTGGAGTGCGAGGACTGTG |
| | AGCTGATCCCGGCGGACCATGTGATTGTGACCGTGTCGCTAGGTGTGCTAAAGAGGCA |
| | GTACACCAGTTTCTTCCGGCCAGGCCTGCCCACAGAGAAGGTGGCTGCCATCCACCGC |
| | CTGGGCATTGGCACCACCGACAAGATCTTTCTGGAATTCGAGGAGCCCTTCTGGGGCC |
| | CTGAGTGCAACAGCCTACAGTTTGTGTGGGAGGACGAAGCAGAGAGCCACACCCTCAC |
| | CTACCCACCTGACCTCTGGTACCGCAAGATCTGCGGCTTTGATGTCCTCTACCCGCCT |
| | GAGCGCTACGGCCATGTGCTGAGCGGCTGGATCTGCGGGGAGGAGGCCCTCGTCATGG |
| | AGAAGTGTGATGACGAGGCAGTGGCCGAGATCTGCACGGAGATGCTGCGTCAGTTCAC |
| | AGGGAACCCCAACATTCCAAAACCTCGGCGAATCTTGCGCTCGGCCTGGGGCAGCAAC |
| | CCTTACTTCCGCGGCTCCTATTCATACACGCAGGTGGGCTCCAGCGGGGCGGATGTGG |
| | AGAAGCTGGCCAAGCCCCTGCCGTACACGGAGAGCTCAAAGACAGCGCCCATGCAGGT |
| | GCTGTTTTCCGGTGAGGCCACCCACCGCAAGTACTATTCCACCACCCACGGTGCTCTG |
| | CTGTCCGGCCAGCGTGAGGCTGCCCGCCTCATTGAGATGTACCGAGACCTCTTCCACC |
| | AGGGGACCTGA |
| | ORF Start: at 29 ORF Stop: TGA at 1691 |
| | SEQ ID NO: 196 554 aa MW at 61687.4kD |
| NOV13o, | QSCESSGDSADDPLSRGLRRRGQPRVVVIGAGLAGLAAAKALLEQGFTDVTVLEASSH |
| CG140122-06 | |
| Protein Sequence | IGGRVQSVKLGHATFELGATWIHGSNGNPIYHLAEANGLLEETTDGERSVGRISLYSK |
| | NGVACYLTNHGRRIPKDVVEEFSDLYNEVYNLTQEFFRHDKPVNAESQNSVGVFTREE |
| | VRNRIRNDPDDPEATKRLKLANIQQYLKVESCESSSHSMDEVSLSAFGEWTEIPGAHH |
| | IIPSGFMRVVELLAEGIPAHVIQLGKPVRCIHWDQASARPRGPEIEPRGEGDHNHDTG |
| | EGGQGGEEPRGGRWDEDEQWSVVVECEDCELIPADHVIVTVSLGVLKRQYTSFFRPGL |
| | PTEKVAAIHRLGIGTTDKIFLEFEEPFWGPECNSLQFVWEDEAESHTLTYPPELWYRK |
| | ICGFDVLYPPERYGHVLSGWICGEEALVNEKCDDEAVAEICTEMLRQFTGNPNIPKPR |
| | RILRSAWGSNPYFRGSYSYTQVGSSGADVEKLAKPLPYTESSKTAPMQVLFSGEATHR |
| | KYYSTTHGALLSGQREAARLIEMYRDLFQQGT |
| | SEQ ID NO: 197 1690 bp |
| NOV13p, | CACCATGCAAAGTTGTGAATCCAGTGGTGACAGTGCGGATGACCCTCTCAGTCGCGGC |
| CG140122-07 | |
| DNA Sequence | CTACGGAGAAGGCGACAGCCTCGTGTGGTGGTGATCGGCGCCGGCTTGGCTGGCCTGG |
| | CTGCAGCCAAAGCACTTCTTGAGCAGGGTTTCACGGATGTCACTGTGCTTGAGGCTTC |
| | CAGCCACATCGGAGGCCGTGTGCAGAGTGTGAAACTTGGACACGCCACCTTTGAGCTG |
| | GGAGCCACCTGGATCCATGGCTCCCATGGGAACCCTATCTATCATCTAGCAGAAGCCA |
| | ACGGCCTCCTGGAAGAGACAACCGATGGGGAACGCAGCGTGGGCCGCATCAGCCTCTA |
| | TTCCAAGAATGGCGTGGCCTGCTACCTTACCAACCACGGCCGCAGGATCCCCAAGGAC |
| | GTGGTTGAGGAATTCAGCGATTTATACAACGAGGTCTATAACTTGACCCAGGAGTTCT |
| | TCCGGCACGATAAACCAGTCAATGCTGAAAGTCAAAATAGCGTGGGGGTGTTCACCCG |
| | AGAGGAGGTGCGTAACCGCATCAGGAATGACCCTGACGACCCAGAGGCTACCAAGCGC |
| | CTCAAGCTCGCCATGATCCAGCAGTACCTGAAGGTGGAGAGCTGTGAGAGCAGCTCAC |
| | ACAGCATGGACGAGGTGTCCCTGAGCGCCTTCGGGGAGTGGACCGAGATCCCCGGCGC |
| | TCACCACATCATCCCCTCGGGCTTCATGCGGGTTGTGGAGCTGCTGGCGGAGGGCATC |
| | CCTGCCCACGTCATCCAGCTAGGGAAACCTGTCCGCTGCATTCACTGGGACCAGGCCT |
| | CAGCCCGCCCCAGAGGCCCTGAGATTGAGCCCCGGGGTGAGGGCGACCACAATCACGA |
| | CACTGGGGAGGGTGGCCAGGGTGGAGAGGAGCCCCGGGGGGGCAGGTGGGATGAGGAT |
| | GAGCAGTGGTCGGTGGTGGTGGAGTGCGAGGACTGTGAGCTGATCCCGGCGGACCATG |
| | TGATTGTGACCGTGTCGCTAGGTGTGCTAAAGAGGCAGTACACCAGTTTCTTCCGGCC |
| | ACGCCTGCCCACAGAGAAGGTGGCTGCCATCCACCGCCTGGGCATTGGCACCACCGAC |
| | AAGATCTTTCTGGAATTCGAGGAGCCCTTCTGGGGCCCTGAGTGCAACAGCCTACAGT |
| | TTGTGTGGGAGGACGAAGCAGAGAGCCACACCCTCACCTACCCACCTGAGCTCTGGTA |
| | CCGCAAGATCTGCGGCTTTGATGTCCTCTACCCGCCTGAGCGCTACGGCCATGTGCTG |
| | AGCGGCTGGATCTGCGGGGAGGAGGCCCTCGTCATGGAGAAGTGTGATGACGAGGCAG |
| | TGGCCGAGATCTGCACGGAGATGCTGCGTCAGTTCACAGGGAACCCCAACATTCCAAA |
| | ACCTCGGCGAATCTTGCCCTCGGCCTGGGGCAGCAACCCTTACTTCCGCGGCTCCTAT |
| | TCATACACGCAGGTGGGCTCCAGCGGGGCGGATGTGGAGAAGCTGGCCAAGCCCCTGC |
| | CGTACACGGAGAGCTCAAAGACAGCGCCCATGCAGGTGCTGTTTTCCGGTGAGGCCAC |
| | CCACCGCAAGTACTATTCCACCACCCACGGTGCTCTGCTGTCCGGCCAGCGTGAGGCT |
| | GCCCCCCTCATTGAGATGTACCGAGACCTCTTCCAGCAGGGGACCCATCATCACCACC |
| | ATCACTGA |
| | ORF Start: at 29 ORF Stop: TGA at 1691 |
| | SEQ ID NO: 196 554 aa MW at 61687.4kD |
| NOV13p | TMQSCESSGDSADDPLSRGLRRRCQPRVVVIGAGLAGLAAAKALLEQGFTDVTVLEAS |
| CG140122-07 | |
| Protein Sequence | SHIGGRVQSVKLGHATFELGATWIHGSHGNPIYHLAEANGLLEETTDGERSVGRISLY |
| | SKNGVACYLTNHGRRIPKDVVEEFSDLYNEVYNLTQEFFRHDKPVNAESQNSVGVFTR |
| | EEVRNRIRNDPDDPEATKRLKLANIQQYLKVESCESSSHSMDEVSLSAFGEWTEIPGA |
| | HHIIPSGFMRVVELLAEGIPAHVIQLGKPVRCIHWDQASARPRGPEIEPRGEGDHNHD |
| | TGEGGQGGEEPRGGRWDEDEQWSVVVECEDCELIPADHVIVTVSLGVLKRQYTSFFRP |
| | GLPTEKVAAIHRLGIGTTDKIFLEFEEPFWGPECNSLQFVWEDEAESHTLTYPPELWY |
| | RKICGFDVLYPPERYGHVLSGWICGEEALVMEKCDDEAVAEICTEMLRQFTGNPNIPK |
| | PRRILRSAWGSNPYFRGSYSYTQVGSSGADVEKLAKPLPYTESSKTAPMQVLFSGEAT |
| | HRKYYSTTHGALLSGQREAARLIEMYRDLFQQGTHHHHHH |
| | SEQ ID NO: 199 1680 bp |
| NOV13q, | TCCACCATGCAAAGTTGTGAATCCAGTGGTGACAGTGCGGATGACCCTCTCAGTCGCG |
| CG140122-08 | |
| DNA Sequence | GCCTACGGAGAAGGGGACAGCCTCGTGTGGTGGTGATCGGCGCCGGCTTGGCTGGCCT |
| | GGCTGCAGCCAAAGCACTTCTTGAGCAGGGTTTCACGGATGTCACTGTGCTTGAGGCT |
| | TCCAGCCACATCGGAGGCCGTGTGCAGAGTGTGAAACTTGGACACGCCACCTTTGAGC |
| | TGGGAGCCACCTGGATCCATGGCTCCCATGGGAACCCTATCTATCATCTAGCAGAAGC |
| | CAACGGCCTCCTGGAAGAGACAACCGATGGGGAACGCAGCGTGGGCCGCATCAGCCTC |
| | TATTCCAAGAATGGCGTGGCCTGCTACCTTACCAACCACGGCCGCAGGATCCCCAAGG |
| | ACGTGGTTGAGGAATTCAGCGATTTATACAACGAGGTCTATAACTTGACCCAGGAGTT |
| | CTTCCGGCACGATAAACCAGTCAATGCTGAAAGTCAAAATAGCGTGGGGGTGTTCACC |
| | CGAGAGGAGGTGCGTAACCGCATCAGGAATGACCCTGACGACCCAGAGGCTACCAAGC |
| | GCCTGAAGCTCGCCATGATCCAGCAGTACCTGAAGGTGGAGAGCTGTGAGAGCAGCTC |
| | ACACAGCATGGACGAGGTGTCCCTGAGCGCCTTCGGGGAGTGGACCGAGATCCCCGGC |
| | GCTCACCACATCATCCCCTCGGGCTTCATGCGGGTTGTGGAGCTGCTGGCGGAGGGCA |
| | TCCCTGCCCACGTCATCCAGCTAGGGAAACCTGTCCGCTGCATTCACTGGGACCAGGC |
| | CTCAGCCCGCCCCAGAGGCCCTGAGATTGAGCCCCGGGGTGAGGGCGACCACAATCAC |
| | GACACTGGGGAGGGTGGCCAGGGTGGAGAGGAGCCCCGGGGGGGCAGGTGGGATGAGG |
| | ATGAGCAGTGGTCGGTGGTGGTGGAGTGCGAGGACTGTGAGCTGATCCCGGCGGACCA |
| | TGTGATTGTGACCGTGTCGCTAGGTGTGCTAAAGAGGCAGTACACCAGTTTCTTCCGG |
| | CCAGGCCTGCCCACAGAGAAGGTGGCTGCCATCCACCGCCTGGGCATTGGCACCACCG |
| | ACAAGATCTTTCTGGAATTCGAGGAGCCCTTCTGGGGCCCTGAGTGCAACAGCCTACA |
| | GTTTGTGTGGGAGGACGAAGCAGAGAGCCACACCCTCACCTACCCACCTGAGCTCTGG |
| | TACCGCAAGATCTCCGGCTTTGATGTCCTCTACCCGCCTGAGCGCTACGGCCATGTGC |
| | TGAGCGGCTGGATCTGCGGGGAGGAGGCCCTCGTCATGGAGAAGTGTGATGACGAGGC |
| | AGTGGCCGAGATCTGCACGGAGATGCTGCGTCAGTTCACAGGGAACCCCAACATTCCA |
| | AAACCTCGGCGAATCTTGCGCTCGGCCTGGGGCAGCAACCCTTACTTCCGCGGCTCCT |
| | ATTCATACACGCAGGTGGGCTCCAGCGGGGCGGATGTGGAGAAGCTGGCCAAGCCCCT |
| | GCCGTACACGGAGAGCTCAAAGACAGCGCCCATGCAGGTGCTGTTTTCCGGTGAGGCC |
| | ACCCACCGCAAGTACTATTCCACCACCCACGGTGCTCTGCTGTCCGGCCAGCGTGAGG |
| | CTGCCCGCCTCATTGAGATGTACCGAGACCTCTTCCAGCAGGGGACCTGAAAGCTT |
| | ORF Start: at 1 ORF Stop: TGA at 1672 |
| | SEQ ID NO: 200 557 aa MW at 62006.8kD |
| NOV13q, | STMQSCESSGDSADDPLSRGLRRRGQPRVVVIGAGLAGLAAAKALLEQGFTDVTVLEA |
| CG140122-08 | |
| Protein Sequence | SSHIGGRVQSVKLGHATFELGATWIHGSHGNPIYHLAEANGLLEETTDGERSVGRISL |
| | YSKNGVACYLTNHGRRIPKDVVEEFSDLYMEVYNLTQEFFRHDKPVNAESQNSVGVFT |
| | REEVRNRIRNDPDDPEATKRLKLAMIQQYLKVESCESSSHSMDEVSLSAFGEWTEIPG |
| | AHHIIPSGFMRVVELLAEGIPAHVIQLGKPVRCIHWDQASARPRGPEIEPRGEGDHNH |
| | DTGEGGQGGEEPRGGRWDEDEQWSVVVECEDCELIPADHVIVTVSLGVLKRQYTSFFR |
| | PGLPTEKVAAIHRLGIGTTDKIFLEFEEPFWGPECNSLQFVWEDEAESHTLTYPPELW |
| | YRKICGFDVLYPPERYGHVLSGWICGEEALVMEKCDDEAVAEICTEMLRQFTGNPNIP |
| | KPRRILRSAWGSNPYFRGSYSYTQVGSSGADVEKLAKPLPYTESSKTAPMQVLFSGEA |
| | THRKYYSTTHGALLSGQREAARLIEMYRDLFQQGT |

Sequence comparison of the above protein sequences yields the following sequence relationships shown in Table 13B.

TABLE 13B
Comparison of NOV13a against NOV13b through NOV13q.

| | | Identities/ |
|---|---|---|
| | | Similarities |
| Protein | NOV13a Residues/ | for the |
| Sequence | Match Residues | Matched Region |
| NOV13b | 1 . . . 280 | 280/280 (100%) |
| | 2 . . . 281 | 280/280 (100%) |
| NOV13c | 1 . . . 555 | 502/585 (85%) |
| | 2 . . . 533 | 502/585 (85%) |
| NOV13d | 2 . . . 555 | 553/554 (99%) |
| | 10 . . . 563 | 553/554 (99%) |
| NOV13e | 1 . . . 555 | 554/555 (99%) |
| | 2 . . . 556 | 554/555 (99%) |
| NOV13f | 1 . . . 555 | 554/555 (99%) |
| | 2 . . . 556 | 554/555 (99%) |
| NOV13g | 1 . . . 555 | 554/555 (99%) |
| | 8 . . . 562 | 554/555 (99%) |
| NOV13h | 1 . . . 555 | 554/555 (99%) |
| | 2 . . . 556 | 554/555 (99%) |
| NOV13i | 1 . . . 555 | 554/555 (99%) |
| | 2 . . . 556 | 554/555 (99%) |
| NOV13j | 2 . . . 555 | 553/554 (99%) |
| | 10 . . . 563 | 553/554 (99%) |
| NOV13k | 1 . . . 555 | 502/555 (90%) |
| | 1 . . . 502 | 502/555 (90%) |
| NOV13l | 1 . . . 280 | 280/280 (100%) |
| | 2 . . . 281 | 280/280 (100%) |
| NOV13m | 1 . . . 555 | 502/585 (85%) |
| | 2 . . . 533 | 502/585 (85%) |
| NOV13n | 1 . . . 555 | 502/555 (90%) |
| | 2 . . . 503 | 502/555 (90%) |
| NOV13o | 2 . . . 555 | 553/554 (99%) |
| | 1 . . . 554 | 553/554 (99%) |
| NOV13p | 1 . . . 555 | 554/555 (99%) |
| | 2 . . . 556 | 554/555 (99%) |
| NOV13q | 1 . . . 555 | 554/555 (99%) |
| | 3 . . . 557 | 554/555 (99% |

Further analysis of the NOV13a protein yielded the following properties shown in Table 13C.

TABLE 13C
Protein Sequence Properties NOV13a

| | |
|---|---|
| PSort analysis: | 0.7900 probability located in plasma membrane; |
| | 0.4802 probability located in microbody |
| | (peroxisome); 0.3000 probability located in |
| | Golgi body; 0.2000 probability located in |
| | endoplasmic reticulum (membrane) |
| SignalP analysis: | Cleavage site between residues 41 and 42 |

A search of the NOV13a protein against the Geneseq database, a proprietary database that contains sequences published in patents and patent publication, yielded several homologous proteins shown in Table 13D.

TABLE 13D
Geneseq Results for NOV13a

| | | | Identities/ | |
|---|---|---|---|---|
| | | | Similarities | |
| Geneseq | Protein/Organism/ | NOV13a Residues/ | for the | Expect |
| Identifier | Length [Patent #, Date] | Match Residues | Matched Region | Value |
| AAB73670 | Human oxidoreductase protein | 1 . . . 555 | 554/555 (99%) | 0.0 |
| | ORP-3 - Homo sapiens, 555 aa. | 1 . . . 555 | 554/555 (99%) | |
| | [WO200144448-A2, 21 JUN. 2001] | | | |
| AAB12164 | Hydrophobic domain protein from | 1 . . . 555 | 554/555 (99%) | 0.0 |
| | clone HP10673 isolated from | 1 . . . 555 | 554/555 (99%) | |
| | Thymus cells - Homo sapiens, | | | |
| | 555 aa. [WO200029448-A2, 25 MAY | | | |
| | 2000] | | | |
| AAM79546 | Human protein SEQ ID NO 3192 - | 1 . . . 510 | 508/510(99%) | 0.0 |
| | Homo sapiens, 518 aa. | 7 . . . 516 | 508/510(99%) | |
| | [WO200157190-A2, 09 AUG. 2001] | | | |
| AAM78562 | Human protein SEQ ID NO 1224 - | 1 . . . 510 | 501/511(98%) | 0.0 |
| | Homo sapiens, 513 aa. | 1 . . . 511 | 501/511(98%) | |
| | [WO200157190-A2, 09 AUG. 2001] | | | |
| AAU21643 | Novel human neoplastic disease | 273 . . . 555 | 282/283 (99%) | e−171 |
| | associated polypeptide #76 - Homo | 53 . . . 335 | 282/283 (99%) | |
| | sapiens, 335 aa. [WO200155163- | | | |
| | A1, 02 AUG. 2001] | | | |

In a BLAST search of public sequence datbases, the NOV13a protein was found to have homology to the proteins shown in the BLASTP data in Table 13E.

TABLE 13E
Public BLASTP Results for NOV13a

| | | | Identities/ | |
|---|---|---|---|---|
| Protein | | | Similarities | |
| Accession | | NOV13a Residues/ | for the | Expect |
| Number | Protein/Organism/Length | Match Residues | Matched Portion | Value |
| Q96QT3 | Polyamine oxidase isoform-1 - | 1 . . . 555 | 555/555 (100%) | 0.0 |
| | Homo sapiens (Human), 555 aa. | 1 . . . 555 | 555/555 (100%) | |
| Q9NWMO | CDNA FLJ20746 fis, clone | 1 . . . 555 | 554/555 (99%) | 0.0 |
| | HEP06040 - Homo sapiens | 1 . . . 555 | 554/555 (99%) | |
| | (Human), 555 aa. | | | |
| Q99K82 | Similar to hypothetical protein - | 1 . . . 554 | 528/554 (95%) | 0.0 |
| | Mus musculus (Mouse), 555 aa. | 1 . . . 554 | 537/554 (96%) | |
| Q9NP51 | DJ779E11.1.5 (Novel flavin | 144 . . . 555 | 411/412 (99%) | 0.0 |
| | containing amine oxidase | 1 . . . 412 | 411/412 (99%) | |
| | (Translation of cDNA | | | |
| | DFKZp761P0724 (Em: AL162058)) | | | |
| | (Isoform 5)) - Homo sapiens | | | |
| | (Human), 412 aa (fragment). | | | |
| Q9H6H1 | CDNA: FLJ22285 fis, clone | 197 . . . 555 | 357/389 (91%) | 0.0 |
| | HRC03956 - Homo sapiens | 1 . . . 389 | 357/389 (91%) | |
| | (Human), 389 aa. | | | |

PFam analysis predicts that the NOV13a protein contains the domains shown in the Table 13F.

TABLE 13F
Domain Analysis of NOV13a

| | | Identities/ | |
|---|---|---|---|
| | | Similarities | |
| | NOV13a Match | for the | Expect |
| Pfam Domain | Region | Matched Region | Value |
| FAD_binding_3 | 27 . . . 141 | 24/142 (17%) | 0.31 |
| | | 74/142 (52%) | |
| Amino_oxidase | 34 . . . 544 | 124/574 (22%) | 1.8e−28 |
| | | 366/574 (64%) | |

The NOV14 clone was analyzed, and the nucleotide and encoded polypeptide sequences are shown in Table 14A.

TABLE 14A
NOV14 Sequence Analysis

| | | | |
|---|---|---|---|
| | SEQ ID NO: 201 2058 bp | | |
| NOV14a, | ATGGAGCCCGAAGCCCCCCGTCGCCGCCACACCCATCAGCGCGGCTACCTGCTGACAC | | |
| CG140316-01 | | | |
| DNA Sequence | GGAACCCTCACCTCAACAACGACTTGGCCTTTACCCTGGAAGAGAGACAGCAATTGAA | | |
| | CATTCATGGATTGTTGCCACCTTCCTTCAACAGTCAGGAGATCCAGGTTCTTAGAGTA | | |
| | GTAAAAAATTTCGAGCATCTGAACTCTGACTTTGACAGGTATCTTCTCTTAATGGATC | | |
| | TCCAAGATAGAAATGAAAAACTCTTTTATAGAGTGCTGACATCTGACATTGAGAAATT | | |
| | CATGCCTATTGTTTATACTCCCACTGTGGGTCTGGCTTGCCAACAATATAGTTTGGTG | | |
| | TTTCGGAAGCCAAGAGGTCTCTTTATTACTATCCACGATCGAGGGCATATTGCTTCAG | | |
| | TTCTCAATGCATGGCCAGAAGATGTCATCAAGGCCATTGTGGTGACTGATGGAGAGCG | | |
| | TATTCTTGGCTTGGGAGACCTTGGCTGTAATGGAATGGGCATCCCTGTGGGTAAATTG | | |
| | GCTCTATATACAGCTTGCGGAGGGATGAATCCTCAAGAATGTCTGCCTGTCATTCTGG | | |
| | ATGTGGGAACCGAAAATGAGGAGTTACTTAAAGATCCACTCTACATTGGACTACGGCA | | |
| | GAGAAGAGTAAGAGGTTCTGAATATGATGATTTTTTGGACGAATTCATGGAGGCAGTT | | |
| | TCTTCCAAGTATGGCATGAATTGCCTTATTCAGTTTGAAGATTTTGCCAATGTGAATG | | |
| | CATTTCGTCTCCTGAACAAGTATCGAAACCAGTATTGCACATTCAATGATGATATTCA | | |
| | AGGAACAGCATCTGTTGCAGTTGCAGGTCTCCTTGCAGCTCTTCGAATAACCAAGAAC | | |
| | AAACTGTCTGATCAAACAATACTATTCCAAGGAGCTGGAGAGGCTGCCCTAGGGATTG | | |
| | CACACCTGATTGTGATGGCCTTGGAAAAAGAAGGTTTACCAAAAGAGAAAGCCATCAA | | |
| | AAAGATATGGCTGGTTGATTCAAAAGGATTAATAGTTAAGGGACGTGCTTCCTTAACA | | |
| | CAAGAGAAAGAGAAGTTTGCCCATGAACATGAAGAAATGAAGAACCTAGAAGCCATTG | | |
| | TTCAAGAAATAAAACCAACTGCCCTCATAGGAGTTGCTGCAATTGGTGGTGCATTCTC | | |
| | AGAACAAATTCTCAAAGATATGGCTGCCTTCAATGAACGGCCTATTATTTTTGCTTTG | | |
| | AGTAATCCAACTAGCAAAGCAGAATGTTCTGCAGAGCAGTGCTACAAAATAACCAAGG | | |
| | GACGTGCAATTTTTGCCAGTGGCAGTCCTTTTGATCCAGTCACTCTTCCAAATGGACA | | |
| | GACCCTATATCCTGGCCAAGGCAACAATTCCTACGTGTTCCCTGGAGTTGCTCTTGGT | | |
| | GTTGTGGCGTGTGGATTGAGGCAGATCACAGATAATATTTTCCTCACTACTGCTGAGG | | |
| | TTATAGCTCAGCAAGTGTCAGATAAACACTTGGAAGAGGGTCGGCTTTATCCTCCTTT | | |
| | GAATACCATTAGAGATGTTTCTCTGAAAATTGCAGAAAAGATTGTGAAAGATGCATAC | | |
| | CAAGAAAAGACAGCCACAGTTTATCCTGAACCGCAAAACAAAGAAGCATTTGTCCGCT | | |
| | CCCAGATGTATAGTACTGATTATGACCAGATTCTACCTGATTGTTATTCTTGGCCTGA | | |
| | AGAGGTGCAGAAAATACAGACCAAAGTTGACCAGTAGGATAATAGCAAACATTTCTAA | | |
| | CTCTATTAATGAGGTCTTTAAACCTTTCATAATTTTTAAAGGTTGGAATCTTTTATAA | | |
| | TGATTCATAAGACACTTAGATTAAGATTTTACTTTAACAGTCTAAAAATTGATAGAAG | | |
| | AATATCGATATAAATTGGGATAAACATCACATGAGACAATTTTGCTTCACTTTGCCTT | | |
| | CTGGTTATTTATGGTTTCTGTCTGAATTATTCTGCCTACGTTCTCTTTAAAAGCTGTT | | |
| | GTACGTACTACGGAGAAACTCATCATTTTTATACAGGACACTAATGGGAAGACCAAAA | | |
| | TTACTAATAAATTGAAATAACCAACATT | | |
| | ORF Start: ATG at 1 ORF Stop: TAG at 1717 | | |
| | SEQ ID NO: 202 1572 aa MW at 64148.9kD | | |
| NOV 14a, | MEPEAPRRRHTHQRGYLLTRNPHLNKDLAFTLEERQQLNIHGLLPPSFNSQEIQVLRV | | |
| CG140316-01 | | | |
| Protein Sequence | VKNFEHLNSDFDRYLLLMDLQDRNEKLFYRVLTSDIEKFMPIVYTPTVGLACQQYSLV | | |
| | FRKPRGLFITIHDRGHIASVLNAWPEDVIKAIVVTDGERILGLGDLGCNGMGIPVGKL | | |
| | ALYTACGGMNPQECLPVILDVGTENEELLKDPLYIGLRQRRVRGSEYDDFLDEFMEAV | | |
| | SSKYGMNCLIQFEDFANVNAFRLLNKYRNQYCTFNDDIQGTASVAVAGLLAALRITKN | | |
| | KLSDQTILFQGAGEAALGIAHLIVMALEKEGLPKEKAIKKIWLVDSKGLIVKGRASLT | | |
| | QEKEKFAHEHEEMKNLEAIVQEIKPTALIGVAAIGGAFSEQILKDMAAFNERPIIFAL | | |
| | SNPTSKAECSAEQCYKITKGPAIFASGSPFDPVTLPNGQTLYPGQGNNSYVFPGVALG | | |
| | VVACGLRQITDNIFLTTAEVIAQQVSDKHLEEGRLYPPLNTIRDVSLKIAEKIVKDAY | | |
| | QEKTATVYPEPQNKEAFVRSQMYSTDYDQILPDCYSWPEEVQKIQTKVDQ | | |
| | SEQ ID NO: 203 2058 bp | | |
| NOV14b, | ATGGAGCCCGAAGCCCCCCGTCGCCGCCACACCCATCAGCGCGGCTACCTGCTGACAC | | |
| CG140316-01 | | | |
| DNA Sequence | GGAACCCTCACCTCAACAAGGACTTGGCCTTTACCCTGGAAGAGAGACAGCAATTGAA | | |
| | CATTCATGGATTGTTGCCACCTTCCTTCAACAGTCAGGAGATCCAGGTTCTTAGAGTA | | |
| | CTAAAAAATTTCGAGCATCTGAACTCTGACTTTGACAGGTATCTTCTCTTAATGGATC | | |
| | TCCAAGATAGAAATGAAAAACTCTTTTATAGAGTGCTGACATCTGACATTGAGAAATT | | |
| | CATGCCTATTGTTTATACTCCCACTGTGGGTCTGGCTTGCCAACAATATAGTTTGGTG | | |
| | TTTCGGAAGCCAAGAGGTCTCTTTATTACTATCCACGATCGAGGGCATATTGCTTCAG | | |
| | TTCTCAATCCATGGCCAGAAGATGTCATCAAGGCCATTGTGGTGACTGATGGAGAGCG | | |
| | TATTCTTGGCTTGGGAGACCTTGGCTGTAATGGAATGGGCATCCCTGTGGGTAAATTG | | |
| | CCTCTATATACAGCTTGCGGAGGGATGAATCCTCAAGAATGTCTGCCTGTCATTCTGG | | |
| | ATGTGGGAACCGAAAATGAGGAGTTACTTAAAGATCCACTCTACATTGGACTACGGCA | | |
| | GAGAAGAGTAAGACGTTCTGAATATGATGATTTTTTGGACGAATTCATGGAGGCAGTT | | |
| | TCTTCCAAGTATGGCATCAATTGCCTTATTCAGTTTGAAGATTTTGCCAATGTGAATG | | |
| | CATTTCGTCTCCTGAACAAGTATCGAAACCAGTATTGCACATTCAATGATGATATTCA | | |
| | AGGAACAGCATCTGTTGCAGTTGCAGGTCTCCTTGCAGCTCTTCGAATAACCAAGAAC | | |
| | AAACTGTCTGATCAAACAATACTATTCCAAGGAGCTGGAGAGGCTGCCCTAGGGATTG | | |
| | CACACCTGATTGTGATGGCCTTGGAAAAAGAAGGTTTACCAAAAGAGAAAGCCATCAA | | |
| | AAAGATATGGCTGGTTGATTCAAAAGGATTAATAGTTAAGGGACCTGCTTCCTTAACA | | |
| | CAAGAGAAAGAGAAGTTTGCCCATGAACATCAAGAAATGAAGAACCTAGAAGCCATTG | | |
| | TTCAAGAAATAAAACCAACTGCCCTCATAGGAGTTGCTGCAATTGGTGGTGCATTCTC | | |
| | AGAACAAATTCTCAAAGATATGGCTGCCTTCAATGAACGGCCTATTATTTTTGCTTTG | | |
| | AGTAATCCAACTAGCAAACCAGAATGTTCTGCAGAGCAGTGCTACAAAATAACCAAGG | | |
| | GACGTGCAATTTTTGCCAGTGGCAGTCCTTTTGATCCAGTCACTCTTCCAAATGGACA | | |
| | GACCCTATATCCTGGCCAAGGCAACAATTCCTACGTGTTCCCTGGAGTTGCTCTTGGT | | |
| | GTTGTGGCGTGTGGATTGAGGCACATCACAGATAATATTTTCCTCACTACTGCTGAGG | | |
| | TTATAGCTCAGCAAGTGTCAGATAAACACTTGGAAGAGGGTCGGCTTTATCCTCCTTT | | |
| | GAATACCATTAGAGATGTTTCTCTGAAAATTGCAGAAAAGATTGTGAAAGATGCATAC | | |
| | CAAGAAAAGACAGCCACAGTTTATCCTGAACCGCAAAACAAAGAAGCATTTGTCCGCT | | |
| | CCCAGATGTATAGTACTGATTATGACCAGATTCTACCTGATTGTTATTCTTGGCCTGA | | |
| | AGAGGTGCAGAAAATACAGACCAAAGTTGACCAGTAGGATAATAGCAAACATTTCTAA | | |
| | CTCTATTAATGAGGTCTTTAAACCTTTCATAATTTTTAAAGGTTGGAATCTTTTATAA | | |
| | TGATTCATAAGACACTTAGATTAAGATTTTACTTTAACAGTCTAAAAATTGATAGAAG | | |
| | AATATCGATATAAATTGGGATAAACATCACATGAGACAATTTTGCTTCACTTTGCCTT | | |
| | CTGGTTATTTATGGTTTCTGTCTGAATTATTCTGCCTACGTTCTCTTTAAAAGCTGTT | | |
| | CTACGTACTACGGAGAAACTCATCATTTTTATACAGGACACTAATGGGAAGACCAAAA | | |
| | TTACTAATAAATTGAAATAACCAACATT | | |
| | ORF Start: ATG at 1 ORF Stop: TAG at 1717 | | |
| | SEQ ID NO: 202 572 aa MW at 64148.9kD | | |
| NOV14b, | MEPEAPRRRHTHQRGYLLTRNPHLNKDLAFTLEERQQLNIHGLLPPSFNSQEIQVLRV | | |
| CG140316-01 | | | |
| Protein Sequence | VKNFEHLNSDFDRYLLLMDLQDRNEKLFYRVLTSDIEKFMPIVYTPTVGLACQQYSLV | | |
| | FRKPRGLFITIHDRGHIASVLNAWPEDVIKAIVVTDGERILGLGDLGCNGMGIPVGKL | | |
| | ALYTACGGMNPQECLPVILDVGTENEELLKDPLYIGLRQRRVRGSEYDDFLDEFMEAV | | |
| | SSKYGMNCLIQFEDFANVNAFRLLNKYRNQYCTFNDDIQGTASVAVAGLLAALRITKN | | |
| | KLSDQTILFQGAGEAALGIAHLIVMALEKEGLPKEKAIKKIWLVDSKGLIVKGRASLT | | |
| | QEKEKFAHEHEEMKNLEAIVQEIKPTALIGVAAIGGAFSEQILKDMAAFNERPIIPAL | | |
| | SNPTSKAECSAEQCYKITKGRATFASGSPFDPVTLPNGQTLYPGQGNNSYVFPGVALG | | |
| | VVACGLRQITDNIFLTTAEVIAQQVSDKHLEEGRLYPPLNTIRDVSLKIAEKIVKDAY | | |
| | QEKTATVYPEPQNKEAFVRSQMYSTDYDQILPDCYSWPEEVQKIQTKVDQ | | |
| | SEQ ID NO: 205 1750 bp | | |
| NOV14c, | CGCGGATCCATGGAGCCCGAAGCCCCCCGTCGCCGCCACACCCATCAGCGCGGCTACC | | |
| 254047949 | | | |
| DNA Sequence | TGCTGACACGGAACCCTCACCTCAACAAGGACTTGGCCTTTACCCTGGAAGAGAGACA | | |
| | GCAATTGAACATTCATGGATTGTTGCCACCTTCCTTCAACAGTCAGGAGATCCAGGTT | | |
| | CTTAGAGTAGTAAAAAATTTCGAGCATCTGAACTCTGACTTTGACAGGTATCTTCTCT | | |
| | TAATGGATCTCCAAGATAGAAATGAAAAACTCTTTTATAGAGTGCTGACATCTGACAT | | |
| | TGAGAAATTCATGCCTATTGTTTATACTCCCACTGTGGGTCTGGCTTGCCAACAATAT | | |
| | AGTTTGGTGTTTCGGAAGCCAAGAGGTCTCTTTATTACTATCCACGATCGAGGGCATA | | |
| | TTGCTTCAGTTCTCAATGCATGGCCAGAAGATGTCATCAAGGCCATTGTGGTGACTGA | | |
| | TGGAGAGCGTATTCTTGGCTTGGGAGACCTTGGCTGTAATGGAATGGGCATCCCTGTG | | |
| | GGTAAATTGGCTCTATATACAGCTTQCGGAGGGATGAATCCTCAAGAATGTCTGCCTG | | |
| | TCATTCTGGATGTGGGAACCGAAAATGAGGAGTTACTTAAAGATCCACTCTACATTGG | | |
| | ACTACGGCAGAGAAGAGTAAGAGGTTCTGAATATGATGATTTTTTGGACGAATTCATG | | |
| | GAGGCAGTTTCTTCCAAGTATGGCATGAATTGCCTTATTCAGTTTGAAGATTTTGCCA | | |
| | ATGTGAATGCATTTCGTCTCCTGAACAAGTATCGAAACCAGTATTGCACATTCAATGA | | |
| | TGATATTCAAGGAACAGCATCTGTTGCAGTTGCAGGTCTCCTTGCAGCTCTTCGAATA | | |
| | ACCAACAACAAACTGTCTGATCAAACAATACTATTCCAAGGAGCTCGGGAGGCTGCCC | | |
| | TAGGGATTGCACACCTGATTGTGATGGCCTTGGAAAAAGAAGGTTTACCAAAAGAGAA | | |
| | AGCCATCAAAAAGATATGGCTGGTTGATTCAAAAGGATTAATAGTTAAGGGACGTGCT | | |
| | TCCTTAACACAAGAGAAAGAGAAGTTTGCCCATGAACATGAAGAAATGAAGAACCTAG | | |
| | AAGCCATTGTTCAAGAAATAAAACCAACTGCCCTCATAGGAGTTGCTGCAATTGGTGG | | |
| | TGCATTCTCAGAACAAATTCTCAAAGATATGGCTGCCTTCAATGAACGGCCTATTATT | | |
| | TTTGCTTTGAGTAATCCAACTAGCAAACCAGAATGTTCTGCAGAGCAGTGCTACAAAA | | |
| | TAACCAAGGGACGTGCAATTTTTGCCAGTGGCAGTCCTTTTGATCCAGTCACTCTTCC | | |
| | AAATGGACAGACCCTATATCCTGGCCAAGGCAACAATTCCTATGTGTTCCCTGGAGTT | | |
| | GCTCTTGGTGTTGTGGCGTGTGGATTGAGGCAGATCACAGATAATATTTTCCTCACTA | | |
| | CTGCTGAGGTTATAGCTCAGCAAGTGTCAGATAAACACTTGGAAGAGGGTCGGCTTTA | | |
| | TCCTCCTTTGAATACCATTAGAGATGTTTCTCTGAAAATTGCAGAAAAGATTGTGAAA | | |
| | GATGCATACCAAGAAAAGACAGCCACAGTTTATCCTGAACCGCAAAACAAAGAAGCAT | | |
| | TTGTCCGCTCCCAGATGTATAGTACTGATTATGACCAGATTCTACCTGATTGTTATTC | | |
| | TTGGCCTGAAGAGGTGCAGAAAATACAGACCAAAGTTGACCAGTAGGGTGGCGGCCGC | | |
| | TTTTTTCCYYTT | | |
| | ORF Start: at 1 ORF Stop: TAG at 1726 | | |
| | SEQ ID NO: 206 575 aa MW at 64449.2kD | | |
| NOV14c, | RGSMEPEAPRRRHTHQRGYLLTRNPHLNKDLAFTLEERQQLNIHGLLPPSFNSQEIQV | | |
| 254047949 | | | |
| Protein Sequence | LRVVKNFEHLNSDFDRYLLLMDLQDRNEKLFYRVLTSDIEKFMPIVYTPTVGLACQQY | | |
| | SLVFRKPRGLFITIHDRGHIASVLNAWPEDVIKAIVVTDGERILGLGDLGCNGMGIPV | | |
| | GKLALYTACGGMNPQECLPVILDVGTENEELLKDPLYIGLRQRRVRGSEYDDFLDEFM | | |
| | EAVSSKYGMNCLIQFEDFANVNAFRLLNKYRNQYCTFNDDIQGTASVAVAGLLAALRI | | |
| | TKNKLSDQTILFQGAGEAALGIAHLIVMALEKEGLPKEKAIKKIWLVDSKGLIVKGRA | | |
| | SLTQEKEKFAHEHEEMKNLEAIVQEIKPTALIGVAAIGGAFSEQILKDMAAFNERPII | | |
| | FALSNPTSKAECSAEQCYKITKGRAIFASGSPFDPVTLPNGQTLYPGQGNNSYVFPGV | | |
| | ALGVVACGLRQITDNIFLTTAEVIAQQVSDKHLEEGRLYPPLNTIRDVSLKIAEKIVK | | |
| | DAYQEKTATVYPEPQNKEAFVRSQMYSTDYDQILPDCYSWPEEVQKIQTKVDQ | | |
| | SEQ ID NO: 207 1752 bp | | |
| NOV14d, | AGCCCGAAGCCCCCCGTCGCCGCCACACCCATCAGCGCCGCTACCTGCTGACACGGAA | | |
| 258280122 | | | |
| DNA Sequence | CCCTCACCTCAACAAGGACTTGGCCTTTACCCTGGAAGAGAGACAGCAATTGAACATT | | |
| | CATGGATTGTTGCCACCTTCCTTCAACAGTCAGGAGATCCAGGTTCTTAGAGTAGTAA | | |
| | AAAATTTCGAGCATCTGAACTCTGACTTTGACAGGTATCTTCTCTTAATGGATCTCCA | | |
| | AGATAGAAATGAAAAACTCTTTTATAGAGTGCTGACATCTGACATTGAGAAATTCATG | | |
| | CCTATTGTTTATACTCCCACTGTGGGTCTGGCTTGCCAACAATATAGTTTGGTGTTTC | | |
| | GGAAGCCAAGAGGTCTCTTTATTACTATCCACGATCGAGGGCATATTGCTTCAGTTCT | | |
| | CAATGCATGGCCAGAAGATGTCATCAAGGCCATTGTGGTGACTGATGGAGAGCGTATT | | |
| | CTTGGCTTGGGAGACCTTGGCTGTAATCGAATGGGCATCCCTGTGGGTAAATTGGCTC | | |
| | TATATACAGCTTGCGGAGGGATGAATCCTCAAGAATGTCTGCCTGTCATTCTGGATGT | | |
| | GGGAACCGAAAATGAGGAGTTACTTAAAGATCCACTCTACATTGGACTACGGCAGAGA | | |
| | AGAGTAAGAGGTTCTGAATATGATGATTTTTTGGACGAATTCATGGAGGCAGTTTCTT | | |
| | CCAAGTATGGCATGAATTGCCTTATTCAGTTTGAAGATTTTGCCAATGTGAATGCATT | | |
| | TCGTCTCCTGAACAAGTATCGAAACCAGTATTGCACATTCAATGATGATATTCAAGGA | | |
| | ACAGCATCTGTTGCAGTTGCAGGTCTCCTTGCAGCTCTTCGAATAACCAAGAACAAAC | | |
| | TGTCTGATCAAACAATACTATTCCAAGGAGCTGGGGAGGCTGCCCTAGGGATTGCACA | | |
| | CCTGATTGTGATGGCCTTGGAAAAAGAAGGTTTACCAAAAGAGAAAGCCATCAAAAAG | | |
| | ATATGGCTGGTTGATTCAAAAGGATTAATAGTTAAGGGACGTGCTTCCTTAACACAAG | | |
| | AGAAAGAGAAGTTTGCCCATGAACATGAAGAAATGAAGAACCTAGAAGCCATTGTTCA | | |
| | AGAAATAAAACCAACTGCCCTCATAGGAGTTGCTGCAATTGGTGGTGCATTCTCAGAA | | |
| | CAAATTCTCAAAGATATGGCTGCCTTCAATGAACGGCCTATTATTTTTGCTTTGAGTA | | |
| | ATCCAACTAGCAAAGCAGAATGTTCTGCAGAGCAGTGCTACAAAATAACCAAGGGACG | | |
| | TGCAATTTTTGCCAGTGGCAGTCCTTTTGATCCAGTCACTCTTCCAAATGGACAGACC | | |
| | CTATATCCTGGCCAAGGCAACAATTCCTATGTGTTCCCTGGAGTTGCTCTTGGTGTTG | | |
| | TGGCGTGTGGATTGAGGCAGATCACAGATAATATTTTCCTCACTACTGCTGAGGTTAT | | |
| | AGCTCAGCAAGTGTCAGATAAACACTTGGAAGACGGTCGGCTTTATCCTCCTTTGAAT | | |
| | ACCATTAGAGATGTTTCTCTGAAAATTGCAGAAAAGATTGTGAAAGATGCATACCAAG | | |
| | AAAAGACAGCCACAGTTTATCCTGAACCGCAAAACAAAGAAGCATTTGTCCGCTCCCA | | |
| | GATGTATAGTACTGATTATGACCAGATTCTACCTGATTGTTATTCTTGGCCTGAAGAG | | |
| | GTGCAGAAAATACAGACCAAAGTTGACCAGTAGGGTGGCGGCCGCACTCGAGCACCAC | | |
| | CACCACCACCAC | | |
| | ORF Start: at 3 ORF Stop: TAG at 1713 | | |
| | SEQ ID NO: 208 570 aa MW at 63888.6kD | | |
| NOV14d, | PEAPRRRHTHQRGYLLTRNPHLNKDLAFTLEERQQLNIHGLLPPSFNSQEIQVLRVVK | | |
| 258280122 | | | |
| Protein Sequence | NFEHLNSDFDRYLLLMDLQDRNEKLFYRVLTSDIEKFMPIVYTPTVGLACQQYSLVFR | | |
| | KPRGLFITIHDRGHIASVLNAWPEDVIKAIVVTDGERILGLGDLGCNGMGIPVGKLAL | | |
| | YTACGGMNPQECLPVILDVGTENEELLKDPLYIGLRQRRVRGSEYDDFLDEFMEAVSS | | |
| | KYGMNCLIQFEDFANVNAFRLLNKYRNQYCTFNDDIQGTASVAVAGLLAALRITKNKL | | |
| | SDQTILFQGAGEAALGIAHLIVMALEKEGLPKEKAIKKIWLVDSKGLIVKGRASLTQE | | |
| | KEKFAHEHEEMKNLEAIVQEIKPTALIGVAAIGGAFSEQILKDMAAFNERPIIFALSN | | |
| | PTSKAECSAEQCYKITKGRAIFASGSPFDPVTLPNGQTLYPGQGNNSYVFPGVALGVV | | |
| | ACGLRQITDNIFLTTAEVTAQQVSDKHLEEGRLYPPLNTIRDVSLKIAEKIVKDAYQE | | |
| | KTATVYPEPQNKEAFVRSQMYSTDYDQILPDCYSWPEEVQKIQTKVDQ | | |
| | SEQ ID NO: 209 1743 bp | | |
| NOV14e, | ACCATGGGCCACCATCACCACCATCACGAGCCCGAAGCCCCCCGTCGCCGCCACACCC | | |
| 258330149 | | | |
| DNA Sequence | ATCAGCGCGGCTACCTGCTGACACGGAACCCTCACCTCAACAAGGACTTGGCCTTTAC | | |
| | CCTGGAAGAGAGACAGCAATTGAACATTCATGCATTGTTGCCACCTTCCTTCAACAGT | | |
| | CAGGAGATCCAGGTTCTTAGAGTAGTAAAAAATTTCGAGCATCTGAACTCTGACTTTG | | |
| | ACAGGTATCTTCTCTTAATGGATCTCCAAGATAGAAATGAAAAACTCTTTTATAGAGT | | |
| | GCTGACATCTGACATTGACAAATTCATGCCTATTGTTTATACTCCCACTGTGGGTCTG | | |
| | GCTTGCCAACAATATAGTTTGGTGTTTCGGAAGCCAAGAGGTCTCTTTATTACTATCC | | |
| | ACGATCGAGGGCATATTGCTTCAGTTCTCAATGCATGGCCAGAAGATGTCATCAAGGC | | |
| | CATTGTGGTGACTGATGCAGAGCGTATTCTTGGCTTGGGAGACCTTGGCTCTAATGGA | | |
| | ATGGGCATCCCTGTGGGTAAATTGGCTCTATATACAGCTTGCGGAGGGATGAATCCTC | | |
| | AAGAATGTCTGCCTGTCATTCTGGATGTGGGAACCGAAAATGAGGAGTTACTTAAAGA | | |
| | TCCACTCTACATTGGACTACGGCAGAGAAGAGTAAGAGGTTCTGAATATGATGATTTT | | |
| | TTGGACGAATTCATGGAGGCAGTTTCTTCCAAGTATGGCATGAATTGCCTTATTCAGT | | |
| | TTGAAGATTTTGCCAATGTGAATGCATTTCGTCTCCTGAACAAGTATCGAAACCAGTA | | |
| | TTGCACATTCAATGATGATATTCAAGGAACAGCATCTGTTGCAGTTGCAGGTCTCCTT | | |
| | GCAGCTCTTCGAATAACCAAGAACAAACTGTCTGATCAAACAATACTATTCCAAGGAG | | |
| | CTGGGGAGGCTGCCCTAGGGATTGCACACCTGATTGTGATGGCCTTGGAAAAAGAAGG | | |
| | TTTACCAAAAGAGAAAGCCATCAAAAAGATATGGCTGGTTGATTCAAAAGGATTAATA | | |
| | GTTAAGGGACGTGCTTCCTTAACACAAGAGAAAGAGAAGTTTGCCCATGAACATGAAG | | |
| | AAATGAAGAACCTAGAAGCCATTGTTCAAGAAATAAAACCAACTGCCCTCATAGGAGT | | |
| | TGCTGCAATTGGTGGTGCATTCTCAGAACAAATTCTCAAAGATATGGCTGCCTTCAAT | | |
| | GAACGGCCTATTATTTTTGCTTTGAGTAATCCAACTAGCAAAGCAGAATGTTCTGCAG | | |
| | AGCAGTGCTACAAAATAACCAAGGGACGTGCAATTTTTGCCAGTGGCAGTCCTTTTGA | | |
| | TCCAGTCACTCTTCCAAATGGACAGACCCTATATCCTGGCCAAGGCAACAATTCCTAT | | |
| | GTGTTCCCTGGAGTTGCTCTTGGTGTTGTGGCGTGTGGATTGAGGCAGATCACAGATA | | |
| | ATATTTTCCTCACTACTGCTGAGGTTATAGCTCAGCAAGTGTCAGATAAACACTTGGA | | |
| | AGAGGGTCGGCTTTATCCTCCTTTGAATACCATTAGAGATGTTTCTCTGAAAATTGCA | | |
| | GAAAAGATTGTGAAAGATGCATACCAAGAAAAGACAGCCACAGTTTATCCTGAACCGC | | |
| | AAAACAAAGAACCATTTGTCCGCTCCCAGATGTATAGTACTGATTATGACCAGATTCT | | |
| | ACCTGATTGTTATTCTTGGCCTGAAGAGGTGCAGAAAATACAGACCAAAGTTGACCAG | | |
| | TAG | | |
| | ORF Start: at 1 | | ORF Stop: TAG at 1741 |
| | SEQ ID NO: 210 | 580 aa | MW at 65129.9kD |
| NOV14e, | TMGHHHHHHEPEAPRRRHTHQRGYLLTRNPHLNKDLAFTLEERQQLNIHGLLPPSFNS | | |
| 258330149 | | | |
| Protein Sequence | QEIQVLRVVKNFEHLNSDFDRYLLLMDLQDRNEKLFYRVLTSDIEKFMPIVYTPTVGL | | |
| | ACQQYSLVFRKPRGLFITIHDRGHIASVLNAWPEDVIKAIVVTDGERILGLGDLGCNG | | |
| | MGIPVGKLALYTACCGMNPQECLPVILDVGTENEELLKDPLYIGLRQRRVRGSEYDDF | | |
| | LDEFMEAVSSKYGMNCLIQFEDFANVNAFRLLNKYRNQYCTFNDDIQGTASVAVAGLL | | |
| | AALRITKNKLSDQTILFQGAGEAALGIAHLIVMALEKEGLPKEKAIKKIWLVDSKGLI | | |
| | VKGRASLTQEKEKFAHEHEEMKNLEAIVQEIKPTALIGVAAIGGAFSEQILKDMAAFN | | |
| | ERPIIFALSNPTSKAECSAEQCYKITKGRAIFASGSPFDPVTLPNGQTLYPGQGNNSY | | |
| | VFPGVALGVVACGLRQITDNIFLTTAEVIAQQVSDKHLEEGRLYPPLNTIRDVSLKIA | | |
| | EKIVKDAYQEKTATVYPEPQNKEAFVRSQMYSTDYDQTLPDCYSWPEEVQKIQTKVDQ | | |
| | SEQ ID NO: 211 1767 bp | | |
| NOV14f | CACCATCACCACCATCACGAGCCCGAAGCCCCCCGTCGCCGCCACACCCATCAGCGCG | | |
| 258330422 | | | |
| DNA Sequence | GCTACCTGCTGACACGGAACCCTCACCTCAACAAGGACTTGGCCTTTACCCTGGAAGA | | |
| | GAGACAGCAATTGAACATTCATGGATTGTTGCCACCTTCCTTCAACAGTCAGGAGATC | | |
| | CAGGTTCTTAGAGTAGTAAAAAATTTCGAGCATCTGAACTCTGACTTTGACAGGTATC | | |
| | TTCTCTTAATGGATCTCCAAGATAGAAATGAAAAACTCTTTTATAGAGTGCTGACATC | | |
| | TGACATTGAGAAATTCATGCCTATTGTTTATACTCCCACTGTGGGTCTGGCTTGCCAA | | |
| | CAATATAGTTTGGTGTTTCGGAAGCCAAGAGGTCTCTTTATTACTATCCACGATCGAG | | |
| | GGCATATTGCTTCAGTTCTCAATCCATGCCCAGAAGATGTCATCAAGGCCATTGTGGT | | |
| | GACTGATGGAGAGCGTATTCTTGGCTTGGGAGACCTTGGCTGTAATGGAATGGGCATC | | |
| | CCTGTGGGTAAATTGGCTCTATATACAGCTTGCGGAGGGATGAATCCTCAAGAATGTC | | |
| | TGCCTGTCATTCTGGATGTGGGAACCGAAAATCAGGAGTTACTTAAAGATCCACTCTA | | |
| | CATTGGACTACGGCAGAGAAGAGTAAGAGGTTCTGAATATGATGATTTTTTGGACGAA | | |
| | TTCATGGAGGCAGTTTCTTCCAAGTATGGCATGAATTGCCTTATTCAGTTTGAAGATT | | |
| | TTGCCAATGTGAATGCATTTCGTCTCCTGAACAAGTATCGAAACCAGTATTGCACATT | | |
| | CAATGATCATATTCAAGGAACAGCATCTGTTGCAGTTGCAGGTCTCCTTGCACCTCTT | | |
| | CGAATAACCAAGAACAAACTGTCTGATCAAACAATACTATTCCAAGGAGCTGGGGAGG | | |
| | CTGCCCTAGGGATTGCACACCTGATTGTGATGGCCTTGGAAAAAGAAGGTTTACCAAA | | |
| | AGAGAAAGCCATCAAAAAGATATGGCTGGTTGATTCAAAAGGATTAATAGTTAAGGGA | | |
| | CGTGCTTCCTTAACACAAGAGAAAGAGAAGTTTGCCCATGAACATGAAGAAATGAAGA | | |
| | ACCTAGAAGCCATTGTTCAAGAAATAAAACCAACTGCCCTCATAGGAGTTGCTGCAAT | | |
| | TGGTGGTGCATTCTCAGAACAAATTCTCAAAGATATGGCTGCCTTCAATGAACGGCCT | | |
| | ATTATTTTTGCTTTGAGTAATCCAACTAGCAAAGCAGAATGTTCTGCAGAGCAGTGCT | | |
| | ACAAAATAACCAAGGGACGTGCAATTTTTGCCAGTGGCAGTCCTTTTGATCCAGTCAC | | |
| | TCTTCCAAATGGACAGACCCTATATCCTGGCCAAGGCAACAATTCCTATGTGTTCCCT | | |
| | GGAGTTGCTCTTGGTGTTGTGGCGTGTGGATTGAGGCAGATCACAGATAATATTTTCC | | |
| | TCACTACTGCTGAGGTTATAGCTCAGCAAGTGTCAGATAAACACTTGGAAGAGGGTCG | | |
| | GCTTTATCCTCCTTTGAATACCATTAGAGATGTTTCTCTGAAAATTGCAGAAAAGATT | | |
| | GTGAAAGATGCATACCAAGAAAAGACAGCCACAGTTTATCCTGAACCGCAAAACAAAG | | |
| | AAGCATTTGTCCGCTCCCAGATGTATAGTACTGATTATGACCAGATTCTACCTGATTG | | |
| | TTATTCTTGGCCTGAAGAGGTGCAGAAAATACAGACCAAAGTTGACCAGTAGGCGGCC | | |
| | GCACTCGAGCACCACCACCACCACCAC | | |
| | ORF Start: at 1 ORF Stop: TAG at 1732 | | |
| | SEQ ID NO: 212 577 aa MW at 64840.6kD | | |
| NOV14f, | HHHHHHEPEAPRRRHTHQRGYLLTRNPHLNKDLAFTLEERQQLNIHGLLPPSFNSQEI | | |
| 258330422 | | | |
| Protein Sequence | QVLRVVKNFEHLNSDFDRYLLLMDLQDRNEKLFYRVLTSDIEKFMPIVYTPTVCLACQ | | |
| | QYSLVFRKPRGLFITIHDRGHIASVLNAWPEDVIKAIVVTDGERILGLGDLGCNGMGI | | |
| | PVGKLALYTACGGMNPQECLPVILDVGTENEELLKDPLYIGLRQRRVRGSEYDDFLDE | | |
| | FMEAVSSKYGMNCLIQFEDFANVNAFRLLNKYRNQYCTFNDDIQGTASVAVAGLLAAL | | |
| | RITKNKLSDQTILFQGAGEAALGIAHLIVMALEKEGLPKEKAIKKIWLVDSKGLIVKG | | |
| | RASLTQEKEKFAHEHEEMKNLEAIVQEIKPTALIGVAAIGGAFSEQILKDMAAFNERP | | |
| | IIFALSNPTSKAECSAEQCYKITKGRAIFASGSPFDPVTLPNGQTLYPGQGNNSYVFP | | |
| | GVALGVVACGLRQITDNIFLTTAEVIAQQVSDKHLEEGRLYPPLNTIRDVSLKIAEKI | | |
| | KDAYQEKTATVYPEPQNKEAFVRSQMYSTDYDQILPDCYSWPEEVQKIQTKVDQ | | |
| | SEQ ID NO: 213 1722 bp | | |
| NOV14g, | ACCATGGAGCCCGAAGCCCCCCGTCGCCGCCACACCCATCAGCGCGGCTACCTGCTGA | | |
| 258330562 | | | |
| DNA Sequence | CACGGAACCCTCACCTCAACAAGGACTTGGCCTTTACCCTGGAAGAGAGACAGCAATT | | |
| | GAACATTCATGGATTGTTGCCACCTTCCTTCAACAGTCAGGAGATCCAGGTTCTTAGA | | |
| | GTAGTAAAAAATTTCGAGCATCTGAACTCTGACTTTGACAGGTATCTTCTCTTAATGG | | |
| | ATCTCCAAGATAGAAATGAAAAACTCTTTTATAGAGTGCTGACATCTGACATTGAGAA | | |
| | ATTCATGCCTATTGTTTATACTCCCACTGTGGGTCTGGCTTGCCAACAATATAGTTTG | | |
| | GTGTTTCGGAAGCCAAGAGGTCTCTTTATTACTATCCACGATCGAGGGCATATTGCTT | | |
| | CAGTTCTCAATGCATGGCCAGAAGATGTCATCAAGGCCATTGTGGTGACTGATGGAGA | | |
| | GCGTATTCTTGGCTTGGGAGACCTTGGCTGTAATGGAATGGGCATCCCTGTGGGTAAA | | |
| | TTGGCTCTATATACAGCTTGCGGAGGGATGAATCCTCAAGAATGTCTGCCTGTCATTC | | |
| | TGGATGTGGGAACCGAAAATGAGGAGTTACTTAAAGATCCACTCTACATTGGACTACG | | |
| | GCAGAGAAGAGTAAGAGGTTCTCAATATGATGATTTTTTGGACGAATTCATGGAGGCA | | |
| | GTTTCTTCCAAGTATGGCATGAATTGCCTTATTCAGTTTGAAGATTTTGCCAATGTGA | | |
| | ATGCATTTCGTCTCCTGAACAAGTATCGAAACCAGTATTGCACATTCAATGATGATAT | | |
| | TCAAGGAACAGCATCTGTTGCAGTTGCAGGTCTCCTTGCAGCTCTTCGAATAACCAAG | | |
| | AACAAACTGTCTGATCAAACAATACTATTCCAAGGAGCTGGAGAGGCTGCCCTAGGGA | | |
| | TTGCACACCTGATTGTGATGGCCTTGGAAAAAGAAGGTTTACCAAAAGAGAAAGCCAT | | |
| | CAAAAAGATATGGCTGGTTGATTCAAAAGGATTAATAGTTAAGGGACGTGCTTCCTTA | | |
| | ACACAAGAGAAAGAGAAGTTTGCCCATGAACATGAAGAAATGAAGAACCTAGAAGCCA | | |
| | TTGTTCAAGAAATAAAACCAACTCCCCTCATAGGAGTTGCTGCAATTGGTGGTGCATT | | |
| | CTCAGAACAAATTCTCAAAGATATGGCTGCCTTCAATGAACGGCCTATTATTTTTGCT | | |
| | TTGAGTAATCCAACTAGCAAAGCAGAATGTTCTGCAGAGCAGTGCTACAAAATAACCA | | |
| | ACGGACGTGCAATTTTTGCCAGTGGCAGTCCTTTTGATCCAGTCACTCTTCCAAATGG | | |
| | ACAGACCCTATATCCTGGCCAAGGCAACAATTCCTATGTGTTCCCTGGAGTTGCTCTT | | |
| | GGTGTTGTGGCGTGTGGATTGAGGCAGATCACAGATAATATTTTCCTCACTACTGCTG | | |
| | AGGTTATAGCTCAGCAAGTGTCAGATAAACACTTGGAAGAGGGTCGGCTTTATCCTCC | | |
| | TTTGAATACCATTAGAGATGTTTCTCTGAAAATTGCAGAAAAGATTGTGAAAGATGCA | | |
| | TACCAAGAAAAGACAGCCACAGTTTATCCTGAACCGCAAAACAAAGAAGCATTTGTCC | | |
| | GCTCCCAGATGTATAGTACTGATTATGACCAGATTCTACCTGATTGTTATTCTTGGCC | | |
| | TGAAGAGGTGCAGAAAATACAGACCAAAGTTGACCAGTAG | | |
| | ORF Start: at 1 ORF Stop: TAG at 1720 | | |
| | SEQ ID NO: 214 573 aa MW at 64250.0kD | | |
| NOV14g, | TMEPEAPRRRHTHQRGYLLTRNPHLNKDLAFTLEERQQLNIHGLLPPSFNSQEIQVLR | | |
| 258330562 | | | |
| Protein Sequence | VVKNFEHLMSDFDRYLLLMDLQDRNEKLFYRVLTSDIEKFMPIVYTPTVGLACQQYSL | | |
| | VFRKPRGLFITIHDRGHIASVLNAWPEDVIKAIVVTDGERILGLGDLGCNGMGIPVGK | | |
| | LALYTACGGMNPQECLPVILDVGTENEELLKDPLYIGLRQRRVRGSEYDDFLDEFMEA | | |
| | VSSKYGMNCLIQFEDFANVNAFRLLNKYRNQYCTFNDDIQGTASVAVAGLLAALRITK | | |
| | NKLSDQTILFQGAGEAALGIAHLIVMALEKEGLPKEKAIKKIWLVDSKGLIVKGRASL | | |
| | TQEKEKFAHEHEEMKNLEAIVQEIKPTALIGVAAIGGAFSEQILKDMAAFNERPIIFA | | |
| | LSNPTSKAECSAEQCYKITKGRAIFASGSPFDPVTLPNGQTLYPGQGNNSYVFPGVAL | | |
| | GVVACGLRQITDNTFLTTAEVIAQQVSDKHLEEGRLYPPLNTIRDVSLKIAEKIVKDA | | |
| | YQEKTATVYPEPQNKEAFVRSQMYSTDYDQILPDCYSWPEEVQKIQTKVDQ | | |
| | SEQ ID NO: 215 1719 bp | | |
| NOV14h, | TGGAGCCCGAAGCCCCCCGTCGCCGCCACACCCATCAGCGCGGCTACCTGCTGACACG | | |
| 258330639 | | | |
| DNA Sequence | GAACCCTCACCTCAACAAGGACTTGGCCTTTACCCTGGAAGAGAGACAGCAATTGAAC | | |
| | ATTCATGGATTGTTGCCACCTTCCTTCAACAGTCAGGAGATCCAGGTTCTTAGAGTAG | | |
| | TAAAAAATTTCGAGCATCTGAACTCTGACTTTGACAGGTATCTTCTCTTAATGGATCT | | |
| | CCAAGATAGAAATGAAAAACTCTTTTATAGAGTGCTGACATCTGACATTGAGAAATTC | | |
| | ATGCCTATTGTTTATACTCCCACTGTGGGTCTGGCTTGCCAACAATATAGTTTGGTGT | | |
| | TTCGGAAGCCAAGAGGTCTCTTTATTACTATCCACGATCGAGGGCATATTGCTTCAGT | | |
| | TCTCAATGCATGGCCAGAAGATGTCATCAAGGCCATTGTGGTGACTGATGGAGAGCGT | | |
| | ATTCTTGGCTTGGGAGACCTTGGCTGTAATGGAATGGGCATCCCTGTGGGTAAATTGG | | |
| | CTCTATATACAGCTTGCGGAGGGATGAATCCTCAAGAATGTCTGCCTGTCATTCTGGA | | |
| | TGTGGGAACCGAAAATGAGGAGTTACTTAAAGATCCACTCTACATTGGACTACGGCAG | | |
| | AGAAGAGTAAGAGGTTCTGAATATGATGATTTTTTGGACGAATTCATGGAGGCAGTTT | | |
| | CTTCCAAGTATGGCATGAATTGCCTTATTCAGTTTGAAGATTTTGCCAATGTGAATGC | | |
| | ATTTCGTCTCCTGAACAAGTATCGAAACCAGTATTGCACATTCAATGATGATATTCAA | | |
| | GGAACAGCATCTGTTGCAGTTGCAGGTCTCCTTGCAGCTCTTCGAATAACCAAGAACA | | |
| | AACTGTCTGATCAAACAATACTATTCCAAGGAGCTGGGGAGGCTGCCCTAGGGATTGC | | |
| | ACACCTGATTGTGATGGCCTTGGAAAAAGAAGGTTTACCAAAAGAGAAAGCCATCAAA | | |
| | AAGATATGGCTGGTTGATTCAAAAGGATTAATAGTTAAGGGACGTGCTTCCTTAACAC | | |
| | AAGAGAAAGAGAAGTTTGCCCATGAACATGAAGAAATGAAGAACCTAGAAGCCATTGT | | |
| | TCAAGAAATAAAACCAACTGCCCTCATAGGAGTTGCTGCAATTGGTGGTGCATTCTCA | | |
| | GAACAAATTCTCAAAGATATGGCTGCCTTCAATGAACGGCCTATTATTTTTGCTTTGA | | |
| | GTAATCCAACTAGCAAAGCAGAATGTTCTGCAGAGCAGTGCTACAAAATAACCAAGGG | | |
| | ACGTGCAATTTTTGCCAGTGGCAGTCCTTTTGATCCAGTCACTCTTCCAAATGGACAG | | |
| | ACCCTATATCCTGGCCAAGGCAACAATTCCTATGTGTTCCCTGGAGTTGCTCTTGGTG | | |
| | TTGTGGCGTGTGGATTGAGGCAGATCACAGATAATATTTTCCTCACTACTGCTGAGGT | | |
| | TATAGCTCAGCAAGTGTCAGATAAACACTTGGAAGAGGGTCGGCTTTATCCTCCTTTG | | |
| | AATACCATTAGAGATGTTTCTCTGAAAATTGCAGAAAAGATTGTGAAAGATGCATACC | | |
| | AAGAAAAGACAGCCACAGTTTATCCTGAACCGCAAAACAAAGAAGCATTTGTCCGCTC | | |
| | CCAGATGTATAGTACTGATTATGACCAGATTCTACCTGATTGTTATTCTTGGCCTGAA | | |
| | GAGGTGCAGAAAATACAGACCAAAGTTGACCAGTAGG | | |
| | ORF Start: at 3 ORF Stop: TAG at 1716 | | |
| | SEQ ID NO: 216 571 aa MW at 64017.7kD | | |
| NOV14h, | EPEAPRRRHTHQRGYLLTRNPHLNKDLAFTLEERQQLNIHGLLPPSFNSQEIQVLRVV | | |
| 258330639 | | | |
| Protein Sequence | KNFEHLNSDFDRYLLLMDLQDRNEKLFYRVLTSDIEKFMPIVYTPTVGLACQQYSLVF | | |
| | RKPRGLFITIHDRGHIASVLNAWPEDVIKAIVVTDGERILGLGDLGCNGMGIPVGKLA | | |
| | LYTACGGMNPQECLPVILDVGTENEELLKDPLYIGLRQRRVRGSEYDDFLDEFMEAVS | | |
| | SKYGMNCLIQFEDFANVNAFRLLNKYRNQYCTFNDDIQGTASVAVAGLLAALRITKNK | | |
| | LSDQTILFQGAGEAALGTAHLIVMALEKEGLPKEKAIKKIWLVDSKGLIVKGRASLTQ | | |
| | EKEKFAHEHEEMKNLEAIVQEIKPTALIGVAAIGGAFSEQILKDMAAFNERPIIFALS | | |
| | NPTSKAECSAEQCYKITKGRAIFASGSPFDPVTLPNGQTLYPGQGNNSYVFPGVALGV | | |
| | VACGLRQITDNIFLTTAEVIAQQVSDKHLEEGRLYPPLNTIRDVSLKIAEKIVKDAYQ | | |
| | EKTATVYPEPQNKEAFVRSQMYSTDYDQILPDCYSWPEEVQKIQTKVDQ | | |
| | SEQ ID NO: 217 1732 bp | | |
| NOV14i, | ACCATGGAGCCCGAAGCCCCCCGTCGCCGCCACACCCATCAGCGCGGCTACCTGCTGA | | |
| 259357792 | | | |
| DNA Sequence | CACGGAACCCTCACCTCAACAAGGACTTGGCCTTTACCCTGGAAGAGAGACAGCAATT | | |
| | GAACATTCATGGATTGTTGCCACCTTCCTTCAACAGTCAGGAGATCCAGGTTCTTAGA | | |
| | GTAGTAAAAAATTTCGAGCATCTGAACTCTGACTTTGACAGGTATCTTCTCTTAATGG | | |
| | ATCTCCAAGATAGAAATGAAAAACTCTTTTATAGAGTGCTGACATCTGACATTGAGAA | | |
| | ATTCATGCCTATTCTTTATACTCCCACTGTGGGTCTGGCTTGCCAACAATATAGTTTG | | |
| | GTGTTTCGGAAGCCAAGAGGTCTCTTTATTACTATCCACGATCGAGGGCATATTGCTT | | |
| | CAGTTCTCAATGCATGGCCAGAAGATGTCATCAAGGCCATTGTGGTGACTGATGGAGA | | |
| | GCGTATTCTTCGCTTGGGAGACCTTGGCTGTAATGGAATGGGCATCCCTGTGGGTAAA | | |
| | TTGGCTCTATATACAGCTTGCGGAGGGATGAATCCTCAAGAATGTCTGCCTGTCATTC | | |
| | TGGATGTGGGAACCGAAAATGAGGAGTTACTTAAAGATCCACTCTACATTGGACTACG | | |
| | GCAGAGAAGAGTAAGAGGTTCTGAATATGATGATTTTTTGGACGAATTCATGGAGGCA | | |
| | GTTTCTTCCAAGTATGCCATGAATTGCCTTATTCAGTTTGAAGATTTTGCCAATGTGA | | |
| | ATGCATTTCGTCTCCTGAACAAGTATCGAAACCAGTATTGCACATTCAATGATGATAT | | |
| | TCAAGGAACAGCATCTGTTGCAGTTGCAGGTCTCCTTGCAGCTCTTCGAATAACCAAG | | |
| | AACAAACTGTCTGATCAAACAATACTATTCCAAGGAGCTGGGGAGGCTGCCCTAGGGA | | |
| | TTGCACACCTGATTGTGATGGCCTTGGAAAAAGAAGGTTTACCAAAAGAGAAAGCCAT | | |
| | CAAAAAGATATGGCTGGTTGATTCAAAAGGATTAATAGTTAAGGGACGTGCTTCCTTA | | |
| | ACACAAGAGAAAGAGAAGTTTGCCCATGAACATGAAGAAATGAAGAACCTAGAAGCCA | | |
| | TTGTTCAAGAAATAAAACCAACTGCCCTCATAGGAGTTGCTGCAATTGGTGGTGCATT | | |
| | CTCAGAACAAATTCTCAAAGATATGGCTGCCTTCAATGAACGGCCTATTATTTTTGCT | | |
| | TTGAGTAATCCAACTAGCAAAGCAGAATGTTCTGCAGAGCAGTGCTACAAAATAACCA | | |
| | AGGGACGTGCAATTTTTGCCAGTGGCAGTCCTTTTGATCCAGTCACTCTTCCAAATGG | | |
| | ACAGACCCTATATCCTGGCCAAGGCAACAATTCCTATGTGTTCCCTGGAGTTGCTCTT | | |
| | GGTGTTGTGGCGTGTGGATTGAGGCAGATCACAGATAATATTTTCCTCACTACTGCTG | | |
| | AGGTTATAGCTCAGCAAGTGTCAGATAAACACTTGGAAGAGGGTCGGCTTTATCCTCC | | |
| | TTTGAATACCATTAGAGATGTTTCTCTGAAAATTGCAGAAAAGATTGTGAAAGATGCA | | |
| | TACCAAGAAAAGACAGCCACAGTTTATCCTGAACCGCAAAACAAAGAAGCATTTGTCC | | |
| | GCTCCCAGATGTATAGTACTGATTATGACCAGATTCTACCTGATTGTTATTCTTGGCC | | |
| | TGAAGAGGTGCAGAAAATACAGACCAAAGTTGACCAGTAGGCGGCCGCTT | | |
| | ORF Start: at 1 ORF Stop: TAG at 1720 | | |
| | SEQ ID NO: 218 573 aa MW at 64250.0kD | | |
| NOV14i, | TMEPEAPRRRHTHQRGYLLTRNPHLNKDLAFTLEERQQLNIHGLLPPSFNSQEIQVLR | | |
| 259357792 | | | |
| Protein Sequence | VVKNFEHLNSDFDRYLLLMDLQDRNEKLFYRVLTSDIEKFMPIVYTPTVGLACQQYSL | | |
| | VFRKPRGLFITIHDRGHIASVLNAWPEDVIKATVVTDGERILGLGDLGCNGMGIPVGK | | |
| | LALYTACGGMNPQECLPVILDVGTENEELLKDPLYIGLRQRRVRGSEYDDFLDEFMEA | | |
| | VSSKYGMNCLIQFEDFANVNAFRLLNKYRNQYCTFNDDIQGTASVAVAGLLAALRITK | | |
| | NKLSDQTILFQGAGEAALGIAHLIVMALEKEGLPKEKAIKKIWLVDSKGLIVKGRASL | | |
| | TQEKEKFAHEHEEMKNLEAIVQEIKPTALIGVAAIGGAFSEQILKDMAAFNERPIIFA | | |
| | LSNPTSKAECSAEQCYKITKGRAIFASGSPFDPVTLPNGQTLYPGQGNNSYVFPGVAL | | |
| | GVVACGLRQITDNIFLTTAEVIAQQVSDKHLEEGRLYPPLNTIRDVSLKIAEKIVKDA | | |
| | YQEKTATVYPEPQNKEAFVRSQMYSTDYDQILPDCYSWPEEVQKIQTKVDQ | | |
| | SEQ ID NO: 219 1838 bp | | |
| NOV14j, | CCGGCGCCAGCCATGGAGCCCGAAGCCCCCCGTCGCCGCCACACCCATCAGCGCCGCT | | |
| CG140316-02 | | | |
| DNA Sequence | ACCTGCTGACACGGAACCCTCACCTCAACAAGGACTTGGCCTTTACCCTGGAAGAGAG | | |
| | ACAGCAATTGAACATTCATGGATTGTTGCCACCTTCCTTCAACAGTCAGGAGATCCAG | | |
| | GTTCTTAGAGTAGTAAAAAATTTCGAGCATCTGAACTCTGACTTTGACAGGTATCTTC | | |
| | TCTTAATGGATCTCCAAGATAGAAATGAAAAACTCTTTTATAGAGTGCTGACATCTGA | | |
| | CATTGAGAAATTCATGCCTATTGTTTATACTCCCACTGTGGGTCTGGCTTGCCAACAA | | |
| | TATAGTTTGGTGTTTCGGAAGCCAAGAGGTCTCTTTATTACTATCCACCATCGAGGGC | | |
| | ATATTGCTTCAGTTCTCAATGCATGGCCAGAAGATGTCATCAAGGCCATTGTGGTGAC | | |
| | TGATGGAGAGCGTATTCTTGGCTTGGGAGACCTTGGCTGTAATGGAATGGGCATCCCT | | |
| | GTGGGTAAATTGGCTCTATATACAGCTTGCGGAGGGATGAATCCTCAAGAATGTCTGC | | |
| | CTGTCATTCTGGATGTGGGAACCGAAAATGAGGAGTTACTTAAAGATCCACTCTACAT | | |
| | TGGACTACGGCAGAGAAGAGTAAGAGGTTCTGAATATGATGATTTTTTGGACGAATTC | | |
| | ATGGAGGCAGTTTCTTCCAAGTATGGCATGAATTGCCTTATTCAGTTTGAAGATTTTG | | |
| | CCAATGTGAATGCATTTCGTCTCCTGAACAAGTATCGAAACCAGTATTGCACATTCAA | | |
| | TGATGATATTCAAGGAACAGCATCTGTTGCAGTTGCAGGTCTCCTTGCAGCTCTTCGA | | |
| | ATAACCAAGAACAAACTGTCTGATCAAACAATACTATTCCAAGGAGCTGGAGAGGCTG | | |
| | CCCTAGGGATTGCACACCTGATTGTGATGGCCTTGGAAAAAGAAGGTTTACCAAAGGA | | |
| | GAAAGCCATCAAAAAGATATGGCTGGTTGATTCAAAAGGATTAATAGTTAAGGGACGT | | |
| | GCTTCCTTAACACAAGAGAAAGAGAAGTTTGCCCATGAACATGAAGAAATGAAGAACC | | |
| | TAGAAGCCATTGTTCAAGAAATAAAACCAACTGCCCTCATAGGAGTTGCTGCAATTGG | | |
| | TGGTGCATTCTCAGAACAAATTCTCAAAGATATGGCTGCCTTCAATGAACGGCCTATT | | |
| | ATTTTTGCTTTGAGTAATCCAACTAGCAAAGCAGAATGTTCTGCAGAGCAGTGCTACA | | |
| | AAATAACCAAGGGACGTGCAATTTTTGCCAGTGGCAGTCCTTTTGATCCAGTCACTCT | | |
| | CCCAGATGGACGGACTCTGTTTCCTGGCCAAGGCAACAATTCCTACGTGTTCCCTGGA | | |
| | GTTGCTCTTGGGGTGGTGGCCTGCGGACTGAGACACATCGATGATAAGGTCTTCCTCA | | |
| | CCACTGCTGAGGTCATATCTCAGCAAGTGTCAGATAAACACCTGCAAGAAGGCCGGCT | | |
| | CTATCCTCCTTTGAATACCATTCGAGACGTTTCGTTGAAAATTGCAGTAAAGATTGTG | | |
| | CAAGATGCATACAAAGAAAAGATGGCCACTGTTTATCCTGAACCCCAAAACAAAGAAG | | |
| | AATTTGTCTCCTCCCAGATGTACAGCACTAATTATGACCAGATCCTACCTGATTGTTA | | |
| | TCCGTGGCCTGCAGAAGTCCAGAAAATACAGACCAAAGTCAACCAGTAACGCAACAGC | | |
| | TAGGATTTTTAACTTTATTAGTAAAATCTTGAAGTTTTCATGATCTTTAAGGGTCAGA | | |
| | ATCTTTTATGATGATTCATAGTATGCTTAGAATAAGGTGC | | |
| | ORF Start: ATG at 13 ORF Stop: TAA at 1729 | | |
| | SEQ ID NO: 220 572 aa MW at 64139.1kD | | |
| NOV14j, | MEPEAPRRRHTHQRGYLLTRNPHLNKDLAFTLEERQQLNIHGLLPPSFNSQEIQVLRV | | |
| CG140316-02 | | | |
| Protein Sequence | VKNFEHLNSDFDRYLLLMDLQDRNEKLFYRVLTSDIEKFMPIVYTPTVGLACQQYSLV | | |
| | FRKPRGLFITIHDRGHIASVLNAWPEDVIKATVVTDGERILGLGDLGCNGMGIPVGKL | | |
| | ALYTACGGMNPQECLPVILDVGTENEELLKDPLYIGLRQRRVRGSEYDDFLDEFMEAV | | |
| | SSKYGMNCLIQFEDFANVNAFRLLNKYRNQYCTFNDDIQGTASVAVAGLLAALRITKN | | |
| | KLSDQTILFQGAGEAALGIAHLIVMALEKEGLPKEKAIKKIWLVDSKGLIVKGRASLT | | |
| | QEKEKFAHEHEEMKNLEAIVQEIKPTALIGVAAIGGAFSEQILKDMAAFNERPIIFAL | | |
| | SNPTSKAECSAEQCYKITKGRAIFASGSPFDPVTLPDGRTLFPGQGNNSYVFPGVALG | | |
| | VVACGLRHIDDKVFLTTAEVTSQQVSDKHLQEGRLYPPLNTIRDVSLKIAVKIVQDAY | | |
| | KEKMATVYPEPQNKEEFVSSQMYSTNYDQILPDCYPWPAEVQKIQTKVNQ | | |
| | SEQ ID NO: 221 1750 bp | | |
| NOV 14k, | CGCGGATCCATGGAGCCCGAAGCCCCCCGTCGCCGCCACACCCATCAGCGCGGCTACC | | |
| CG140316-03 | | | |
| DNA Sequence | TGCTGACACGGAACCCTCACCTCAACAAGGACTTGGCCTTTACCCTGGAAGAGAGACA | | |
| | GCAATTGAACATTCATGGATTGTTGCCACCTTCCTTCAACAGTCAGGAGATCCAGGTT | | |
| | CTTAGAGTAGTAAAAAATTTCGAGCATCTGAACTCTGACTTTGACAGGTATCTTCTCT | | |
| | TAATGGATCTCCAAGATAGAAATGAAAAACTCTTTTATAGAGTGCTGACATCTGACAT | | |
| | TGAGAAATTCATGCCTATTGTTTATACTCCCACTGTGGGTCTGGCTTGCCAACAATAT | | |
| | AGTTTGGTGTTTCGGAAGCCAAGAGGTCTCTTTATTACTATCCACGATCGAGGGCATA | | |
| | TTGCTTCAGTTCTCAATGCATGGCCAGAAGATGTCATCAAGGCCATTGTGGTGACTGA | | |
| | TGGAGAGCGTATTCTTGGCTTGGGAGACCTTGGCTGTAATGGAATGGGCATCCCTGTG | | |
| | GGTAAATTGGCTCTATATACAGCTTGCGGAGGGATGAATCCTCAAGAATGTCTGCCTG | | |
| | TCATTCTGGATGTGGGAACCGAAAATGAGGAGTTACTTAAAGATCCACTCTACATTGG | | |
| | ACTACGGCAGAGAAGAGTAAGAGGTTCTGAATATGATGATTTTTTGGACGAATTCATG | | |
| | GAGGCAGTTTCTTCCAAGTATGGCATGAATTGCCTTATTCAGTTTGAAGATTTTGCCA | | |
| | ATGTGAATGCATTTCGTCTCCTGAACAAGTATCGAAACCAGTATTGCACATTCAATGA | | |
| | TGATATTCAAGGAACAGCATCTGTTGCAGTTGCAGGTCTCCTTGCAGCTCTTCGAATA | | |
| | ACCAAGAACAAACTGTCTGATCAAACAATACTATTCCAAGGAGCTGGGGAGGCTGCCC | | |
| | TAGGGATTGCACACCTGATTGTGATGGCCTTGGAAAAAGAAGGTTTACCAAAAGAGAA | | |
| | AGCCATCAAAAAGATATGGCTGGTTGATTCAAAAGGATTAATAGTTAACGGACGTGCT | | |
| | TCCTTAACACAAGAGAAAGAGAAGTTTGCCCATGAACATGAAGAAATGAAGAACCTAG | | |
| | AAGCCATTGTTCAAGAAATAAAACCAACTGCCCTCATAGGAGTTGCTGCAATTGGTGG | | |
| | TGCATTCTCAGAACAAATTCTCAAAGATATGGCTGCCTTCAATGAACGGCCTATTATT | | |
| | TTTGCTTTGAGTAATCCAACTAGCAAAGCAGAATGTTCTGCAGAGCAGTGCTACAAAA | | |
| | TAACCAAGGGACGTGCAATTTTTGCCAGTGGCAGTCCTTTTGATCCAGTCACTCTTCC | | |
| | AAATGGACACACCCTATATCCTGGCCAAGGCAACAATTCCTATGTGTTCCCTGGAGTT | | |
| | GCTCTTGGTGTTGTGGCGTGTGGATTGAGGCAGATCACAGATAATATTTTCCTCACTA | | |
| | CTGCTGAGGTTATAGCTCAGCAAGTGTCAGATAAACACTTGGAAGAGGGTCGGCTTTA | | |
| | TCCTCCTTTGAATACCATTAGAGATGTTTCTCTGAAAATTGCAGAAAAGATTGTGAAA | | |
| | GATGCATACCAAGAAAAGACAGCCACAGTTTATCCTGAACCGCAAAACAAAGAAGCAT | | |
| | TTGTCCGCTCCCAGATGTATAGTACTGATTATGACCAGATTCTACCTGATTGTTATTC | | |
| | TTGGCCTGAAGAGGTGCAGAAAATACAGACCAAAGTTGACCAGTAGGGTGCCGGCCGC | | |
| | TTTTTTCCTT | | |
| | ORF Start: ATG at 10 ORF Stop: TAG at 1726 | | |
| | SEQ ID NO: 222 572 aa MW at 64148.9kD | | |
| NOV14k, | MEPEAPRRRHTHQRGYLLTRNPHLNKDLAFTLEERQQLNIHGLLPPSFNSQEIQVLRV | | |
| CG140316-03 | | | |
| Protein Sequence | VKNFEHLNSDFDRYLLLMDLQDRNEKLFYRVLTSDIEKFMPIVYTPTVGLACQQYSLV | | |
| | FRKPRGLFITIHDRGHIASVLNAWPEDVIKAIVVTDGERILGLGDLGCNGMGIPVGKL | | |
| | ALYTACGGMNPQECLPVILDVGTENEELLKDPLYIGLRQRRVRGSEYDDFLDEFMEAV | | |
| | SSKYGMNCLIQFEDFANVNAFRLLNKYRNQYCTFNDDIQGTASVAVAGLLAALRITKN | | |
| | KLSDQTILFQGAGEAALGIAHLIVMALEKEGLPKEKAIKKIWLVDSKGLIVKGRASLT | | |
| | QEKEKFAHEHEEMKNLEAIVQEIKPTALIGVAAIGGAFSEQILKDMAAFNERPIIFAL | | |
| | SNPTSKAECSAEQCYKITKGRAIFASGSPFDPVTLPNGQTLYPGQGNNSYVFPGVALG | | |
| | VVACGLRQITDNIFLTTAEVIAQQVSDKHLEEGRLYPPLNTIRDVSLKIAEKIVKDAY | | |
| | QEKTATVYPEPQNKEAFVRSQMYSTDYDQILPDCYSWPEEVQKIQTKVDQ | | |
| | SEQ ID NO: 223 1767 bp | | |
| NOV14l, | CACCATCACCACCATCACGAGCCCGAAGCCCCCCGTCGCCGCCACACCCATCAGCGCG | | |
| CG140316-04 | | | |
| DNA Sequence | GCTACCTGCTGACACGGAACCCTCACCTCAACAAGGACTTGGCCTTTACCCTGGAAGA | | |
| | GAGACAGCAATTGAACATTCATGGATTGTTGCCACCTTCCTTCAACAGTCAGGAGATC | | |
| | CAGGTTCTTAGAGTAGTAAAAAATTTCGAGCATCTGAACTCTGACTTTGACAGGTATC | | |
| | TTCTCTTAATGGATCTCCAAGATAGAAATGAAAAACTCTTTTATAGAGTGCTGACATC | | |
| | TGACATTGAGAAATTCATGCCTATTGTTTATACTCCCACTGTGGGTCTGGCTTGCCAA | | |
| | CAATATAGTTTGGTGTTTCGGAAGCCAAGAGGTCTCTTTATTACTATCCACGATCGAG | | |
| | GGCATATTGCTTCAGTTCTCAATGCATGGCCAGAAGATGTCATCAAGGCCATTGTGGT | | |
| | GACTGATGGAGAGCGTATTCTTGGCTTGGGAGACCTTCGCTCTAATOGAATGGCCATC | | |
| | CCTGTGGGTAAATTGGCTCTATATACAGCTTGCGGAGGGATGAATCCTCAAGAATGTC | | |
| | TGCCTGTCATTCTGGATGTGGGAACCGAAAATGAGGAGTTACTTAAAGATCCACTCTA | | |
| | CATTGGACTACGGCAGAGAAGAGTAAGAGGTTCTGAATATGATGATTTTTTGGACGAA | | |
| | TTCATGGAGGCAGTTTCTTCCAAGTATGGCATGAATTGCCTTATTCAGTTTGAAGATT | | |
| | TTGCCAATGTGAATGCATTTCGTCTCCTGAACAAGTATCGAAACCAGTATTGCACATT | | |
| | CAATGATGATATTCAAGGAACAGCATCTGTTGCAGTTGCAGGTCTCCTTGCAGCTCTT | | |
| | CGAATAACCAAGAACAAACTGTCTGATCAAACAATACTATTCCAAGGAGCTGGGGAGG | | |
| | CTGCCCTAGGGATTGCACACCTGATTGTGATGGCCTTGGAAAAAGAAGGTTTACCAAA | | |
| | AGAGAAAGCCATCAAAAAGATATGGCTGGTTGATTCAAAAGGATTAATAGTTAAGGGA | | |
| | CGTGCTTCCTTAACACAAGAGAAAGAGAAGTTTGCCCATGAACATGAAGAAATGAAGA | | |
| | ACCTAGAAGCCATTGTTCAAGAAATAAAACCAACTGCCCTCATAGGAGTTGCTGCAAT | | |
| | TGGTGGTGCATTCTCAGAACAAATTCTCAAAGATATGGCTGCCTTCAATGAACGGCCT | | |
| | ATTATTTTTGCTTTGAGTAATCCAACTAGCAAAGCAGAATGTTCTGCAGAGCAGTGCT | | |
| | ACAAAATAACCAAGGGACGTGCAATTTTTGCCAGTGGCAGTCCTTTTGATCCAGTCAC | | |
| | TCTTCCAAATGGACAGACCCTATATCCTGGCCAAGGCAACAATTCCTATGTGTTCCCT | | |
| | GGAGTTGCTCTTGGTGTTGTGGCGTGTGGATTGAGGCAGATCACAGATAATATTTTCC | | |
| | TCACTACTGCTGAGGTTATAGCTCAGCAAGTGTCAGATAAACACTTCGAAGAGGGTCG | | |
| | GCTTTATCCTCCTTTGAATACCATTAGAGATGTTTCTCTGAAAATTGCAGAAAAGATT | | |
| | GTGAAAGATGCATACCAAGAAAAGACAGCCACAGTTTATCCTGAACCGCAAAACAAAG | | |
| | AAGCATTTGTCCGCTCCCAGATGTATAGTACTGATTATGACCAGATTCTACCTGATTC | | |
| | TTATTCTTGGCCTGAAGAGGTGCAGAAAATACAGACCAAAGTTGACCAGTAGGCGGCC | | |
| | CCACTCGAGCACCACCACCACCACCAC | | |
| NOV14l, | HHHHHHEPEAPRRRHTHQRGYLLTRNPHLNKDLAFTLEERQQLNIHGLLPPSFNSQEI | | |
| CG140316-04 | | | |
| Protein Sequence | QVLRVVKNFEHLNSDFDRYLLLMDLQDRNEKLFYRVLTSDIEKFMPIVYTPTVGLACQ | | |
| | QYSLVFRKPRGLFITIHDRGHIASVLNAWPEDVIKAIVVTDGERILGLGDLGCNGMGI | | |
| | PVGKLALYTACGGMNPQECLPVILDVGTENEELLKDPLYIGLRQRRVRGSEYDDFLDE | | |
| | FMEAVSSKYGMNCLIQFEDFANVNAFRLLNKYRNQYCTFNDDIQGTASVAVAGLLAAL | | |
| | RITKNKLSDQTILFQGAGEAALGIAHLIVMALEKEGLPKEKAIKKIWLVDSKGLIVKG | | |
| | RASLTQEKEKFAHEHEEMKNLEAIVQEIKPTALIGVAAIGGAFSEQILKDMAAFNERP | | |
| | IIFALSNPTSKAECSAEQCYKITKGRAIFASGSPFDPVTLPNGQTLYPGQGNNSYVFP | | |
| | GVALGVVACGLRQITDNIFLTTAEVIAQQVSDKHLEEGRLYPPLNTIRDVSLKIAEKI | | |
| | VKDAYQEKTATVYPEPQNKEAFVRSQMYSTDYDQILPDCYSWPEEVQKIQTKVDQ | | |

Sequence comparison of the above protein sequences yields the following sequence relationships shown in Table 14B.

TABLE 14B
Comparison of NOV14a against NOV14b through NOV14l.

| | | Identities/ | |
|---|---|---|---|
| | | Similarities for | |
| Protein | NOV14a Residues/ | the Matched | |
| Sequence | Match Residues | Region | |
| NOV14b | 1 . . . 572 | 572/572 | (100%) |
| | 1 . . . 572 | 572/572 | (100%) |
| NOV14c | 1 . . . 572 | 572/572 | (100%) |
| | 4 . . . 575 | 572/572 | (100%) |
| NOV14d | 3 . . . 572 | 570/570 | (100%) |
| | 1 . . . 570 | 570/570 | (100%) |
| NOV14e | 2 . . . 572 | 571/571 | (100%) |
| | 10 . . . 580 | 571/571 | (100%) |
| NOV14f | 2 . . . 572 | 571/571 | (100%) |
| | 7 . . . 577 | 571/571 | (100%) |
| NOV14g | 1 . . . 572 | 572/572 | (100%) |
| | 2 . . . 573 | 572/572 | (100%) |
| NOV14h | 2 . . . 572 | 571/571 | (100%) |
| | 1 . . . 571 | 571/571 | (100%) |
| NOV14i | 1 . . . 572 | 572/572 | (100%) |
| | 2 . . . 573 | 572/572 | (100%) |
| NOV14j | 1 . . . 572 | 553/572 | (96%) |
| | 1 . . . 572 | 563/572 | (97%) |
| NOV14k | 1 . . . 572 | 572/572 | (100%) |
| | 1 . . . 572 | 572/572 | (100%) |
| NOV14l | 2 . . . 572 | 571/571 | (100%) |
| | 7 . . . 577 | 571/571 | (100%) |

Further analysis of the NOV14a protein yielded the following properties shown in Table 14C.

TABLE 14C
Protein Sequence Properties NOV14a

| | |
|---|---|
| PSort | 0.7000 probability located in nucleus; 0.3000 |
| analysis: | probability located in microbody (peroxisome); |
| | 0.1771 probability located in lysosome (lumen); |
| | 0.1000 probability located in mitochondrial |
| | matrix space |
| SignalP | No Known Signal Sequence Predicted |
| analysis: | |

A search of the NOV14a protein against the Geneseq database, a proprietary database that contains sequences published in patents and patent publication, yielded several homologous proteins shown in Table 14D.

TABLE 14D
Geneseq Results for NOV14a

| | | | Identities/ | | |
|---|---|---|---|---|---|
| | | | Similarities for | | |
| Geneseq | Protein/Organism/Length | NOV14a Residues/ | the Matched | | Expect |
| Identifier | [Patent #, Date] | Match Residues | Region | | Value |
| AAR52605 | Human cytoplasmic NADP+- | 1 . . . 572 | 572/572 | (100%) | 0.0 |
| | dependent malate enzyme ME1 - | 1 . . . 572 | 572/572 | (100%) | |
| | Homo sapiens, 572 aa. [EP595241- | | | | |
| | A, 04 MAY 1994] | | | | |
| AAM40228 | Human polypeptide SEQ ID NO | 13 . . . 568 | 404/556 | (72%) | 0.0 |
| | 3373 - Homo sapiens, 604 aa. | 48 . . . 603 | 485/556 | (86%) | |
| | [WO200153312-A1, 26 JUL. | | | | |
| | 2001] | | | | |
| AAU33270 | Novel human secreted protein | 13 . . . 568 | 380/563 | (67%) | 0.0 |
| | #3761 - Homo sapiens. 621 aa. | 58 . . . 620 | 464/563 | (81%) | |
| | [WO200179449-A2, 25 OCT. | | | | |
| | 2001] | | | | |
| AAM42014 | Human polypeptide SEQ ID NO | 13 . . . 568 | 376/566 | (66%) | 0.0 |
| | 6945 - Homo sapiens, 624 aa. | 58 . . . 623 | 458/566 | (80%) | |
| | [WO200153312-A1, 26 JUL. | | | | |
| | 2001] | | | | |
| ABG21889 | Novel human diagnostic protein | 13 . . . 568 | 372/567 | (65%) | 0.0 |
| | #21880 - Homo sapiens, 625 aa. | 58 . . . 624 | 455/567 | (79%) | |
| | [WO200175067-A2, 11 OCT. | | | | |
| | 2001] | | | | |

In a BLAST search of public sequence datbases, the NOV14a protein was found to have homology to the proteins shown in the BLASTP data in Table 14E.

TABLE 14E
Public BLASTP Results for NOV14a

| | | | Identities/ | | |
|---|---|---|---|---|---|
| Protein | | | Similarities for | | |
| Accession | | NOV14a Residues/ | the Matched | | Expect |
| Number | Protein/Organism/Length | Match Residues | Portion | | Value |
| P48163 | NADP-dependent malic enzyme | 1 . . . 572 | 572/572 | (100%) | 0.0 |
| | (EC 1.1.1.40) (NADP-ME) (Malic | 1 . . . 572 | 572/572 | (100%) | |
| | enzyme 1) - Homo sapiens | | | | |
| | (Human), 572 aa. | | | | |
| Q16797 | NADP-dependent malic enzyme | 1 . . . 572 | 553/572 | (96%) | 0.0 |
| | (EC 1.1.1.40) - Homo sapiens | 1 . . . 572 | 563/572 | (97%) | |
| | (Human), 572 aa. | | | | |
| JC4160 | malate dehydrogenase | 1 . . . 572 | 552/572 | (96%) | 0.0 |
| | (oxaloacetate-decarboxylating) | 1 . . . 572 | 562/572 | (97%) | |
| | (NADP+) (EC 1.1.1.40) - human, | | | | |
| | 572 aa. | | | | |
| P13697 | NADP-dependent malic enzyme | 1 . . . 572 | 517/572 | (90%) | 0.0 |
| | (EC 1.1.1.40) (NADP-ME) (Malic | 1 . . . 572 | 549/572 | (95%) | |
| | enzyme 1) - Rattus norvegicus | | | | |
| | (Rat), 572 aa. | | | | |
| Q921S3 | Malic enzyme, supernatant - Mus | 1 . . . 572 | 516/572 | (90%) | 0.0 |
| | musculus (Mouse), 572 aa. | 1 . . . 572 | 545/572 | (95%) | |

PFam analysis predicts that the NOV14a protein contains the domains shown in the Table 14F.

TABLE 14F
Domain Analysis of NOV14a

| | | | |
|---|---|---|---|
| | | Identities/ | |
| | | Similarities for | |
| Pfam | NOV14a | the Matched | Expect |
| Domain | Match Region | Region | Value |

| | | | | |
|---|---|---|---|---|
| Paramyx_ncap | 278 . . . 314 | 14/37 | (38%) | 0.77 |
| | | 24/37 | (65%) | |
| malic | 15 . . . 553 | 356/580 | (61%) | 0 |
| | | 515/580 | (89%) | |

### Example 15

The NOV15 clone was analyzed, and the nucleotide and encoded polypeptide sequences are shown in Table 15A.

TABLE 15A
NOV15 Sequence Analysis

| | |
|---|---|
| | SEQ ID NO: 225 4427 bp |
| NOV15a, | GGCACGAGGCCGGGACAAAAGCCGGATCCCGGGAAGCTACCGGCTGCTGGGGTGCTCC |
| CG142427-01 | |
| DNA Sequence | GGATTTTGCGGGGTTCGTCGGGCCTGTGGAAGAAGCGCCGCGCACGGACTTCGGCAGA |
| | GGTAGAGCAGGTCTCTCTGCAGCCATGTCGGCCAAGGCAATTTCAGAGCAGACGGGCA |
| | AACAACTCCTTTACAAGTTCATCTGTACCACCTCAGCCATCCAGAATCGGTTCAAGTA |
| | TGCTCGGGTCACTCCTGACACAGACTGGGCCCGCTTGCTGCAGGACCACCCCTGGCTG |
| | CTCAGCCAGAACTTGGTAGTCAAGCCAGACCAGCTGATCAAACGTCGTGGAAAACTTG |
| | GTCTCGTTGGGGTCAACCTCACTCTCGATGGGGTCAAGTCCTGGCTGAAGCCACGGCT |
| | GGGACAGGAAGCCACAGTTGGCAAGGCCACAGGCTTCCTCAAGAACTTTCTGATCGAG |
| | CCCTTCGTCCCCCACAGTCAGGCTGAGGAGTTCTATGTCTGCATCTATCCCACCCGAG |
| | AAGGGGACTACGTCCTGTTCCACCACGAGGGGGGTGTGGACGTCGGTGATGTGGACGC |
| | CAAGGCCCAGAAGCTGCTTGTTGCCGTGGATGAGAAACTGAATCCTGAGGACATCAAA |
| | AAACACCTGTTGGTCCACGCCCCTGAAGACAAGAAAGAAATTCTGGCCAGTTTTATCT |
| | CCCGCCTCTTCAATTTCTACGAGGACTTGTACTTCACCTACCTCGAGATCAATCCCCT |
| | TGTAGTGACCAAAGATGGAGTCTATGTCCTTGACTTGGCGGCCAAGGTGGACGCCACT |
| | GCCGACTACATCTGCAAAGTGAAGTGGGGTGACATCGAGTTCCCTCCCCCCTTCGGGC |
| | GGGAGGCATATCCAGAGGAAGCCTACATTGCAGACCTCGATGCCAAAAGTGGGGCAAG |
| | CCTGAAGCTGACCTTGCTGAACCCCAAAGGGAGGATCTGGACCATGGTGGCCGGGGGT |
| | GGCGCCTCTGTCGTGTACAGCGATACCATCTGTGATCTAGGGGGTGTCAACGAGCTGG |
| | CAAACTATGGGGAGTACTCAGGCGCCCCCAGCGAGCAGCAGACCTATGACTATGCCAA |
| | GACTATCCTCTCCCTCATGACCCGAGAGAAGCACCCAGATGGCAAGATCCTCATCATT |
| | GGAGGCACCATCGCAAACTTCACCAACGTGGCTGCCACGTTCAAGGGCATCGTGAGAG |
| | CAATTCGAGATTACCAGGGCCCCCTGAAGGAGCACGAAGTCACAATCTTTGTCCGAAG |
| | AGGTGGCCCCAACTATCAGGAGGGCTTACGGGTGATGGGAGAAGTCGGGAAGACCACT |
| | GGGATCCCCATCCATGTCTTTGGCACAGAGACTCACATGACGGCCATTGTGGGCATGG |
| | CCCTGGGCCACCGGCCCATCCCCAACCAGCCACCCACAGCGGCCCACACTGCAAACTT |
| | CCTCCTCAACGCCAGCGGGAGCACATCGACCCCAGCCCCCAGCAGGACAGCATCTTTT |
| | TCTGAGTCCAGGGCCGATGAGGTGGCGCCTGCAAAGAAGGCCAAGCCTGCCATGCCAC |
| | AAGATTCAGTCCCAAGTCCAAGATCCCTGCAAGGAAAGAGCACCACCCTCTTCAGCCG |
| | CCACACCAAGGCCATTGTGTGGGGCATGCAGACCCGGGCCGTGCAAGGCATGCTGGAC |
| | TTTGACTATGTCTGCTCCCGAGACGAGCCCTCAGTGGCTGCCATGGTCTACCCTTTCA |
| | CTGGGGACCACAAGCAGAAGTTTTACTGGGGGCACAAAGAGATCCTGATCCCTGTCTT |
| | CAAGAACATGGCTGATGCCATGAGGAAGCATCCGGAGGTAGATGTGCTCATCAACTTT |
| | GCCTCTCTCCGCTCTGCCTATGACAGCACCATGGAGACCATCAACTATGCCCAGATCC |
| | GGACCATCGCCATCATAGCTGAAGGCATCCCTGAGGCCCTCACGAGAAAGCTGATCAA |
| | GAAGGCGGACCAGAAGGCAGTGACCATCATCGGACCTGCCACTGTTGGAGGCATCAAG |
| | CCTGGGTGCTTTAAGATTGGCAACACAGGTGGGATGCTGGACAACATCCTGGCCTCCA |
| | AACTGTACCGCCCAGGCAGCGTGGCCTATGTCTCACGTTCCGGAGGCATGTCCAACGA |
| | GCTCAACAATATCATCTCTCGGACCACGGATGGCGTCTATGAGGGCGTGGCCATTGGT |
| | GGGGACAGGTACCCGGGCTCCACATTCATGGATCATGTGTTACGCTATCAGGACACTC |
| | CAGGAGTCAAAATGATTGTGGTTCTTGGAGAGATTGGGGGCACTGAGGAATATAAGAT |
| | TTGCCGGGGCATCAAGGACGGCCGCCTCACTAAGCCCATCGTCTGCTGGTGCATCGGG |
| | ACGTGTGCCACCATGTTCTCCTCTGAGGTCCAGTTTGGCCATGCTGGAGCTTGTGCCA |
| | ACCAGGCTTCTGAAACTGCAGTAGCCAAGAACCAGGCTTTGAAGGAAGCAGGAGTGTT |
| | TGTGCCCCGGAGCTTTGATGAGCTTGGAGAGATCATCCAGTCTGTATACGAAGATCTC |
| | GTGGCCAATGGAGTCATTGTACCTGCCCAGGAGGTGCCGCCCCCAACCGTGCCCATGG |
| | ACTACTCCTGGGCCAGGGAGCTTGGTTTGATCCGCAAACCTGCCTCGTTCATGACCAG |
| | CATCTGCGATGAGCGAGGACAGGAGCTCATCTACGCGGGCATGCCCATCACTGAGGTC |
| | TTCAAGGAAGAGATGGGCATTGGCGGGGTCCTCGGCCTCCTCTGGTTCCAGAAAAGGT |
| | TGCCTAAGTACTCTTGCCAGTTCATTGACATGTGTCTGATGGTGACAGCTGATCACGG |
| | GCCAGCCGTCTCTGGAGCCCACAACACCATCATTTGTGCGCGAGCTGGGAAAGACCTG |
| | GTCTCCAGCCTCACCTCGGGGCTGCTCACCATCGGGGATCGGTTTGGGGGTGCCTTGG |
| | ATGCAGCAGCCAAGATGTTCAGTAAAGCCTTTGACAGTGGCATTATCCCCATGGAGTT |
| | TGTGAACAAGATGAAGAAGGAAGGGAAGCTGATCATGGGCATTGGTCACCGAGTGAAG |
| | TCCATAAACAACCCAGACATGCGAGTGCAGATCCTCAAAGATTACGTCAGGCAGCACT |
| | TCCCTGCCACTCCTCTGCTCGATTATGCACTGGAAGTAGAGAAGATTACCACCTCGAA |
| | GAAGCCAAATCTTATCCTGAATGTAGATGGTCTCATCGGAGTCGCATTTGTAGACATG |
| | CTTAGAAACTGTGGGTCCTTTACTCGGGAGGAAGCTGATGAATATATTGACATTGGAG |
| | CCCTCAATGGCATCTTTGTGCTGGGAAGGAGTATGGGGTTCATTGGACACTATCTTGA |
| | TCAGAAGAGGCTGAAGCAGGGGCTGTATCGTCATCCGTGGGATGATATTTCATATGTT |
| | CTTCCGGAACACATGAGCATGTAACAGAGCCAGGAACCCTACTGCAGTAAACTGAACA |
| | CAAGATCTCTTCCCCCAAGAAAAAGTGTACAGACAGCTGGCAGTGGAGCCTGCTTTAT |
| | TTAGCAGGGGCCTGGAATGTAAACAGCCACTGGGGTACAGGCACCGAAGACCAACATC |
| | CACAGGCTAACACCCCTTCAGTCCACACAAAGAACCTTCATATTTTTTTTATAAGCAT |
| | AGAAATAAAAACCAAGCCAATATTTGTGACTTTGCTCTGCTACCTGCTGTATTTATTA |
| | TATGGAAGCATCTAAGTACTGTCAGGATGGGGTCTTCCTCATTGTAGGGCGTTAGGAT |
| | GTTGCTTTCTTTTTCCATTAGTTAAACATTTTTTTCTCCTTTGGAGGAAGGGAATGAA |
| | ACATTTATGGCCTCAAGATACTATACATTTAAAGCACCCCAATGTCTCTCTTTTTTTT |
| | TTTTTACTTCCCTTTCTTCTTCCTTATATAACATGAAGAACATTGTATTAATCTGATT |
| | TTTAAAGATCTTTTTGTATGTTACGTGTTAAGGGCTTGTTTGGTATCCCACTGAAATG |
| | TTCTGTGTTGCAGACCAGAGTCTGTTTATGTCAGGGGGATGGGGCCATTGCATCCTTA |
| | GCCATTGTCACAAAATATGTGGAGTAGTAACTTAATATGTAAAGTTGTAACATACATA |
| | CATTTAAAATGGAAATGCAGAAAGCTGTGAAATGTCTTGTGTCTTATGTTCTCTGTAT |
| | TTATGCAGCTGATTTGTCTGTCTGTAACTGAAGTGTGGGTCCAAGGACTCCTAACTAC |
| | TTTGCATCTGTAATCCACAAAGATTCTGGGCAGCTGCCACCTCAGTCTCTTCTCTGTA |
| | TTATCATAGTCTGGTTTAAATAACTATATAGTAACAAAAAAAAAAAAAAAAAAAAAA |
| | AAAAAAAAAAAAAAAAAAAAAAAAAAAAAAAAAAAAAAAAAAAAAAAAAAAAAAAAAA |
| | AAAAAAAAAAAAAAAAAAA |
| | ORF Start: ATG at 141 ORF Stop: TAA at 3444 |
| | SEQ ID NO: 226 1101 aa MW at 120838.0kD |
| NOV15a, | MSAKAISEQTGKELLYKFICTTSAIQNRFKYARVTPDTDWARLLQDHPWLLSQNLVVK |
| CG142427-01 | |
| Protein Sequence | PDQLIKRRGKLGLVGVNLTLDGVKSWLKPRLGQEATVGKATGFLKNFLIEPFVPHSQA |
| | EEFYVCIYATREGDYVLFHHEGGVDVGDVDAKAQKLLVGVDEKLNPEDIKKHLLVHAP |
| | EDKKEILASFISGLFNFYEDLYFTYLEINPLVVTKDGVYVLDLAAKVDATADYICKVK |
| | WGDIEFPPPFGREAYPEEAYIADLDAKSGASLKLTLLNPKGRIWTMVAGGGASVVYSD |
| | TICDLGGVNELANYGEYSGAPSEQQTYDYAKTILSLMTREKHPDGKILIIGGSIANFT |
| | NVAATFKGIVRAIRDYQGPLKEHEVTIFVRRGGPNYQEGLRVMGEVGKTTGIPIHVFG |
| | TETHMTAIVGMALGHRPIPNQPPTAAHTANFLLNASGSTSTPAPSRTASFSESRADEV |
| | APAKKAKPAMPQDSVPSPRSLQGKSTTLFSRHTKAIVWGMQTRAVQGMLDFDYVCSRD |
| | EPSVAAMVYPFTGDHKQKFYWGHKEILIPVFKNMADAMRKHPEVDVLINFASLRSAYD |
| | STMETMNYAQIRTIAIIAEGIPEALTRKLIKKADQKGVTIIGPATVGGIKPGCFKIGN |
| | TGGMLDNILASKLYRPGSVAYVSRSGGMSNELNNIISRTTDGVYEGVAIGGDRYPGST |
| | FMDHVLRYQDTPGVKMIVVLGEIGGTEEYKICRGIKEGRLTKPIVCWCIGTCATMFSS |
| | EVQFGHAGACANQASETAVAKNQALKEAGVFVPRSFDELGEIIQSVYEDLVANGVIVP |
| | AQEVPPPTVPMDYSWARELGLIRKPASFMTSICDERGQELIYAGMPITEVFKEEMGIG |
| | GVLGLLWFQKRLPKYSCQFIEMCLMVTADHGPAVSGAHNTIICARAGKDLVSSLTSGL |
| | LTIGDRFGGALDAAAKNFSKAFDSGIIPMEFXTNKMKKEGKLIMGIGHRVKSINNPDMR |
| | VQILKDYVRQHFPATPLLDYALEVEKITTSKKPNLILNVDGLIGVAFVDMLRNCGSFT |
| | REEADEYIDIGALNGIFVLGRSMGFIGHYLDQKRLKQGLYRHPWDDISYVLPEHMSM |
| | SEQ ID NO: 227 4427 bp |
| NOV15b, | GGCACGAGGCCGGGACAAAAGCCGGATCCCGGGAAGCTACCGGCTGCTGGGGTGCTCC |
| CG142427-01 | |
| DNA Sequence | GGATTTTGCGGGGTTCGTCGGGCCTGTGGAAGAAGCGCCGCGCACGGACTTCGGCAGA |
| | GGTAGAGCAGGTCTCTCTGCAGCCATGTCGGCCAAGGCAATTTCAGAGCAGACGGGCA |
| | AAGAACTCCTTTACAAGTTCATCTGTACCACCTCAGCCATCCAGAATCGGTTCAAGTA |
| | TGCTCGGGTCACTCCTGACACAGACTGGGCCCGCTTGCTGCAGGACCACCCCTGGCTG |
| | CTCAGCCAGAACTTGGTAGTCAAGCCAGACCAGCTGATCAAACGTCGTGGAAAACTTG |
| | GTCTCGTTGGGGTCAACCTCACTCTGGATGGGGTCAAGTCCTGGCTGAAGCCACGGCT |
| | GGGACAGGAAGCCACAGTTGGCAAGGCCACAGGCTTCCTCAAGAACTTTCTGATCGAG |
| | CCCTTCGTCCCCCACAGTCAGGCTGAGGAGTTCTATGTCTGCATCTATGCCACCCGAG |
| | AAGGGGACTACGTCCTGTTCCACCACGAGGGGGGTGTGGACGTGGGTGATGTGGACGC |
| | CAAGGCCCAGAAGCTGCTTGTTGGCGTGGATGAGAAACTGAATCCTGAGGACATCAAA |
| | AAACACCTGTTGGTCCACGCCCCTGAAGACAAGAAAGAAATTCTGGCCAGTTTTATCT |
| | CCGGCCTCTTCAATTTCTACGAGGACTTGTACTTCACCTACCTCGAGATCAATCCCCT |
| | TGTAGTGACCAAAGATGGAGTCTATGTCCTTGACTTGGCGGCCAAGGTGGACGCCACT |
| | GCCGACTACATCTGCAAAGTGAAGTGGGGTGACATCGAGTTCCCTCCCCCCTTCGGGC |
| | GGGAGGCATATCCAGAGGAAGCCTACATTGCAGACCTCGATGCCAAAAGTGGGGCAAG |
| | CCTGAAGCTGACCTTGCTGAACCCCAAAGGGAGGATCTGGACCATGGTGGCCGGGGGT |
| | GGCGCCTCTGTCGTGTACAGCGATACCATCTGTGATCTAGGGGGTGTCAACGAGCTGG |
| | CAAACTATGGGGAGTACTCAGGCGCCCCCAGCGAGCAGCAGACCTATGACTATGCCAA |
| | GACTATCCTCTCCCTCATGACCCGAGAGAAGCACCCAGATGGCAAGATCCTCATCATT |
| | GGAGGCAGCATCGCAAACTTCACCAACGTGGCTGCCACGTTCAAGGGCATCGTGAGAG |
| | CAATTCGAGATTACCAGGGCCCCCTGAAGGAGCACGAAGTCACAATCTTTGTCCGAAG |
| | AGGTGGCCCCAACTATCAGGAGGGCTTACGGGTGATGGGAGAAGTCGGGAAGACCACT |
| | GGGATCCCCATCCATGTCTTTGGCACAGAGACTCACATGACGGCCATTGTGGGCATGG |
| | CCCTGGGCCACCGGCCCATCCCCAACCAGCCACCCACAGCGGCCCACACTGCAAACTT |
| | CCTCCTCAACGCCAGCGGGAGCACATCGACGCCAGCCCCCAGCAGGACAGCATCTTTT |
| | TCTGAGTCCAGGGCCGATGAGGTGGCGCCTGCAAAGAAGGCCAAGCCTGCCATGCCAC |
| | AACATTCAGTCCCAAGTCCAAGATCCCTGCAAGGAAAGAGCACCACCCTCTTCAGCCG |
| | CCACACCAAGGCCATTGTGTGGGGCATGCAGACCCGGGCCGTGCAAGGCATGCTGGAC |
| | TTTGACTATGTCTGCTCCCGAGACGAGCCCTCAGTGGCTGCCATGGTCTACCCTTTCA |
| | CTGGGGACCACAAGCAGAAGTTTTACTGGGGGCACAAAGAGATCCTGATCCCTGTCTT |
| | CAAGAACATGGCTGATGCCATGAGGAAGCATCCGGAGGTAGATGTGCTCATCAACTTT |
| | GCCTCTCTCCGCTCTGCCTATGACAGCACCATGGAGACCATGAACTATGCCCAGATCC |
| | GGACCATCGCCATCATAGCTGAAGGCATCCCTGAGGCCCTCACGAGAAAGCTGATCAA |
| | GAAGGCGGACCAGAAGCGAGTGACCATCATCGGACCTGCCACTGTTGGAGGCATCAAG |
| | CCTGGGTGCTTTAAGATTGGCAACACAGGTGGGATGCTGGACAACATCCTGGCCTCCA |
| | AACTGTACCGCCCAGGCAGCGTGGCCTATGTCTCACGTTCCGGAGGCATGTCCAACGA |
| | GCTCAACAATATCATCTCTCGGACCACGGATGGCGTCTATGAGGGCGTGGCCATTGGT |
| | GGGGACAGGTACCCGGGCTCCACATTCATGGATCATGTGTTACGCTATCAGGACACTC |
| | CAGGAGTCAAAATGATTGTGGTTCTTGGAGAGATTGGGGGCACTGAGGAATATAAGAT |
| | TTGCCGGGGCATCAAGGAGGGCCGCCTCACTAAGCCCATCGTCTGCTGGTGCATCGGG |
| | ACGTGTGCCACCATGTTCTCCTCTGAGGTCCAGTTTGGCCATGCTGGAGCTTGTGCCA |
| | ACCACGCTTCTGAAACTGCAGTAGCCAAGAACCAGGCTTTGAAGGAAGCAGGAGTGTT |
| | TGTGCCCCGGAGCTTTGATGAGCTTGGAGAGATCATCCAGTCTGTATACGAAGATCTC |
| | GTGGCCAATGGAGTCATTGTACCTGCCCAGGAGGTGCCGCCCCCAACCGTGCCCATGG |
| | ACTACTCCTGGGCCAGGGAGCTTGGTTTGATCCGCAA~ACCTGCCTCGTTCATGACCAG |
| | CATCTGCGATGAGCGAGGACAGGAGCTCATCTACGCGGGCATGCCCATCACTGAGGTC |
| | TTCAAGGAAGAGATGGGCATTGGCGGGGTCCTCGGCCTCCTCTGGTTCCAGAAAAGGT |
| | TGCCTAAGTACTCTTGCCAGTTCATTGAGATGTGTCTGATGGTGACAGCTGATCACGG |
| | GCCAGCCGTCTCTGGAGCCCACAACACCATCATTTGTGCGCGAGCTGGGAAAGACCTG |
| | GTCTCCAGCCTCACCTCGGGGCTGCTCACCATCGGGGATCGGTTTGGGGGTGCCTTOG |
| | ATGCAGCAGCCAAGATGTTCAGTAAAGCCTTTGACAGTGGCATTATCCCCATGGAGTT |
| | TGTGAACAAGATGAAGAAGGAAGGGAAGCTGATCATGGGCATTGGTCACCGAGTGAAG |
| | TCGATAAACAACCCAGACATCCGAGTGCAGATCCTCAAAGATTACGTCAGGCAGCACT |
| | TCCCTGCCACTCCTCTGCTCGATTATGCACTGGAAGTAGAGAAGATTACCACCTCGAA |
| | GAAGCCAAATCTTATCCTGAATGTAGATGGTCTCATCGGAGTCGCATTTGTAGACATG |
| | CTTAGAAACTGTGGGTCCTTTACTCGGGAGGAAGCTGATGAATATATTGACATTGGAG |
| | CCCTCAATGGCATCTTTGTGCTGGGAAGGAGTATGGCGTTCATTGGACACTATCTTGA |
| | TCAGAAGAGGCTGAAGCAGGGGCTGTATCGTCATCCGTGGGATGATATTTCATATGTT |
| | CTTCCGGAACACATGAGCATGTAACAGAGCCAGGAACCCTACTGCAGTAAACTGAAGA |
| | CAAGATCTCTTCCCCCAAGAAAAAGTGTACAGACAGCTGGCAGTGGAGCCTGCTTTAT |
| | TTAGCAGGGGCCTGGAATGTAAACAGCCACTGGGGTACAGGCACCGAAGACCAACATC |
| | CACAGGCTAACACCCCTTCAGTCCACACAAAGAAGCTTCATATTTTTTTTATAAGCAT |
| | AGAAATAAAAACCAAGCCAATATTTGTGACTTTGCTCTGCTACCTGCTGTATTTATTA |
| | TATGGAAGCATCTAAGTACTGTCAGGATGGGGTCTTCCTCATTGTAGGGCGTTAGGAT |
| | GTTGCTTTCTTTTTCCATTAGTTAAACATTTTTTTCTCCTTTGGAGGAAGGGAATGAA |
| | ACATTTATGGCCTCAAGATACTATACATTTAAAGCACCCCAATCTCTCTCTTTTTTTT |
| | TTTTTACTTCCCTTTCTTCTTCCTTATATAACATGAAGAACATTGTATTAATCTGATT |
| | TTTAAAGATCTTTTTGTATGTTACGTGTTAAGGGCTTGTTTGGTATCCCACTGAAATG |
| | TTCTGTGTTGCAGACCAGAGTCTGTTTATGTCAGGGGGATGGGGCCATTGCATCCTTA |
| | GCCATTGTCACAAAATATGTGGAGTACTAACTTAATATGTAAAGTTGTAACATACATA |
| | CATTTAAAATGGAAATGCAGAAAGCTGTGAAATGTCTTGTGTCTTATGTTCTCTGTAT |
| | TTATGCAGCTGATTTGTCTGTCTGTAACTGAAGTGTGGGTCCAAGGACTCCTAACTAC |
| | TTTGCATCTGTAATCCACAAAGATTCTGGGCAGCTGCCACCTCAGTCTCTTCTCTGTA |
| | TTATCATAGTCTGGTTTAATAAACTATATAGTAACAAAAAAAAAAAAAAAAAAAAAA |
| | AAAAAAAAAAAAAAAAAAAAAAAAAAAAAAAAAAAAAAAAAAAAAAAAAAAAAAAAAA |
| | AAAAAAAAAAAAAAAAAAA |
| | ORF Start: ATG at 141 ORF Stop: TAA at 3444 |
| | SEQ ID NO: 228 1101 aa MW at 120838.0kD |
| NOV15b, | MSAKAISEQTGKELLYKFICTTSAIQNRFKYARVTPDTDWARLLQDHPWLLSQNLVVK |
| CG142427-01 | |
| Protein Sequence | PDQLIKRRGKLGLVGVNLTLDGVKSWLKPRLGQEATVGKATGFLKNFLIEPFVPHSQA |
| | EEFYVCIYATREGDYVLFHHEGGVDVGDVDAKAQKLLVGVDEKLNPEDIKKHLLVHAP |
| | EDKKEILASFISGLFNFYEDLYFTYLEINPLVVTKDGVYVLDLAAKVDATADYICKVK |
| | WGDIEFPPPFGREAYPEEAYIADLDAKSGASLKLTLLNPKGRIWTMVAGGGASVVYSD |
| | TICDLGGVNELANYGEYSGAPSEQQTYDYAKTILSLMTREKHPDGKILIIGGSIANFT |
| | NVAATFKGIVRAIRDYQGPLKEHEVTIFVRRGGPNYQEGLRVMGEVGKTTGIPIHVFG |
| | TETHMTAIVGMALGHRPIPNQPPTAAHTANFLLNASGSTSTPAPSRTASFSESRADEV |
| | APAKKAKPAMPQDSVPSPRSLQGKSTTLFSRHTKAIVWGMQTRAVQGMLDFDYVCSRD |
| | EPSVAAMVYPFTGDHKQKFYWGHKEILIPVFKNMADAMRKHPEVDVLINFASLRSAYD |
| | STMETMNYAQIRTIAIIAEGIPEALTRKLIKKADQKGVTIIGPATVGGIKPGCFKIGN |
| | TGGMLDNILASKLYRPGSVAYVSRSGGMSNELNNIISRTTDGVYEGVAIGGDRYPGST |
| | FMDHVLRYQDTPGVKMIVVLGEIGGTEEYKICRGIKEGRLTKPIVCWCIGTCATMFSS |
| | EVQFGHAGACANQASETAVAKNQALKEAGVFVPRSFDELGEIIQSVYEDLVANGVIVP |
| | AQEVPPPTVPMDYSWARELGLIRKPASFMTSICDERGQELIYAGMPITEVFKEEMGIG |
| | GVLGLLWFQKRLPKYSCQFIEMCLMVTADHGPAVSGAHNTIICARAGKDLVSSLTSGL |
| | LTIGDRFGGALDAAAKMFSKAFDSGIIPMEFVNKMKKEGKLIMGIGHRVKSINNPDMR |
| | VQILKDYVRQHFPATPLLDYALEVEKITTSKKPNLILNVDGLIGVAFVDMLRNCGSFT |
| | REEADEYIDIGALNGIFVLGRSMGFIGHYLDQKRLKQGLYRHPWDDISYVLPEHMSM |
| | SEQ ID NO: 229 3238 bp |
| NOV15c, | CCAGAATTCCACCATGTCGGCCAACGCAATTTCAGAGCAGACGGGCAAAGAACTCCTT |
| CG142427-04 | |
| DNA Sequence | TACAAGTTCATCTGTACCACCTCAGCCATCCAGAATCGGTTCAAGTATGCTCGGGTCA |
| | CTCCTGACACAGACTGGGCCCGCTTGCTCCAGGACCACCCCTGGCTGCTCAGCCAGAA |
| | CTTGGTAGTCAAGCCACACCAGCTGATCAAACGTCGTGGAAAACTTGGTCTCGTTGGG |
| | GTCAACCTCACTCTGGATGGGGTCAAGTCCTGGCTGAAGCCACGGCTGGGACAGGAAG |
| | CCACAGTTGGCAAGGCCACAGGCTTCCTCAAGAACTTTCTGATCGAGCCCTTCGTCCC |
| | CCACAGTCAGGCTGAGGAGTTCTATGTCTGCATCTATGCCACCCGAGAAGGGGACTAC |
| | GTCCTGTTCCACCACGAGGGGGGTGTGGACGTGGGTCATGTGGACGCCAAGGCCCAGA |
| | AGCTGCTTGTTGGCGTGGATGAGAAACTGAATCCTGAGGACATCAAAAAACACCTGTT |
| | GGTCCACGCCCCTGAAGACAAGAAAGAAATTCTGGCCAGTTTTATCTCCGGCCTCTTC |
| | AATTTCTACGAGGACTTGTACTTCACCTACCTCGAGATCAATCCCCTTGTAGTGACCA |
| | AAGATGGAGTCTATGTCCTTGACTTGGCGGCCAAGGTGGACGCCACTGCCGACTACAT |
| | CTGCAAAGTGAAGTGGGGTGACATCGAGTTCCCTCCCCCCTTCGGGCGGGAGGCATAT |
| | CCAGAGGAAGCCTACATTGCAGGCCTCGATGCCAAAAGTGGGGCAAGCCTGAAGCTGA |
| | CCTTGCTGAACCCCAAAGGGAGGATCTGGACCATGGTGGCCGGGGGTGGCGCCTCTGT |
| | CGTGTACAGCGATACCATCTGTGATCTAGGGGGTGTCAACGAGCTGGCAAACTATGGG |
| | GAGTACTCAGGCGCCCCCAGCGAGCAGCAGACCTATGACTATGCCAAGACTATCCTCT |
| | CCCTCATGACCCGAGAGAAGCACCCAGATGGCAAGATCCTCATCATTGGAGGCAGCAT |
| | CGCAAACTTCACCAACGTGGCTGCCACGTTCAAGGGCATCGTGAGAGCAATTCGAGAT |
| | TACCAGGGCCCCCTGAAGGAGCACGAAGTCACAATCTTTGTCCGAAGAGGTGGCCCCA |
| | ACTATCAGGAGGGCTTACGGGTGATGGGAGAAGTCGGGAAGACCACTGGGATCCCCAT |
| | CCATGTCTTTGGCACAGAGACCCACACTGCAAACTTCCTCCTCAACGCCAGCGGGAGC |
| | ACATCGACGCCAGCCCCCAGCAGGACAGCATCTTTTTCTGAGTCCAGGGCCGATGAGG |
| | TGGCGCCTGCAAAGAAGGCCAAGCCTGCCATGCCACAAGGAAAGAGCACCACCCTCTT |
| | CAGCCGCCACACCAAGGCCATTGTGTGGGGCATGCAGACCCGGGCCGTGCAAGGCATG |
| | CTGGACTTTGACTATGTCTGCTCCCGAGACGAGCCCTCAGTGGCTGCCATGGTCTACC |
| | CTTTCACTGGGGACCACAAGCAGAAGTTTTACTGGGGGCACAAAGAGATCCTGATCCC |
| | TGTCTTCAAGAACATGGCTGATGCCATGAGGAAGCATCCGGAGGTAGATGTGCTCATC |
| | AACTTTGCCTCTCTCCGCTCTGCCTATGACAGCACCATGGAGACCACGAACTATGCCC |
| | AGATCCGGACCATCGCCATCATAGCTGAAGGCATTCCTGAGGCCCTCACGAGAAAGCT |
| | GATCAAGAAGGCGGACCAGAAGGGAGTGACCATCATCGGACCTGCCACTGTTGGAGGC |
| | ATCAAGCCTGGGTGCTTTAAGATTGGCAACACAGGTGGGATGCTGGACAACATCCTGG |
| | CCTCCAAACTGTACCGCCCAGGCAGCGTGGCCTATGCCTCACGTTCCCGAGGCATGTC |
| | CAACGAGCTCAACAATATCATCTCTCGGACCACGGATGGCGTCTATGAGGGCGTGGCC |
| | ATTGGTGGGGACAGGTACCCGGGCTCCACATTCATGGATCATGTGTTACGCTATCAGG |
| | ACACTCCAGGAGTCAAAATGATTGTGGTTCTTGGAGAGATTGGGGGCACTGAGGAATA |
| | TAAGATTTGCCGGGGCATCAAGGAGGGCCGCCTCACTAAGCCCATCGTCTGCTGGTGC |
| | ATCCGGACGTGTGCCACCATGTTCTCCTCTGAGGTCCAGTTTGGCCATGCTGGAGCTT |
| | CTGCCAACCAGGCTTCTGAAACTGCAGTAGCCAAGAACCAGGCTTTGAAGGAACCAGC |
| | AGTGTTTGTGCCCCGGAGCTTTGATGAGCTTGGAGAGATCATCCAGTCTGTATACGAA |
| | GATCTCGTGGCCAATGGAGTCATTGTACCTGCCCAGGAGGTGCCGCCCCCAACCGTGC |
| | CCATGGACTACTCCTGGGCCAGGGAGCTTGGTTTGATCCGCAAACCTGCCTCGTTCAT |
| | GACCAGCATCTGCGATGAGCGAGGACAGGAGCTCATCTACGCGGGCATGCCCATCACT |
| | GAGGTCTTCAAGGAAGAGATGGGCATTGGCGGGGTCCTCGGCCTCCTCTGGTTCCAGA |
| | AAAGGTTGCCTAAGTACTCTTGCCAGTTCATTGAGATGTGTCTGATGGTGACAGCTGA |
| | TCACGGGCCAGCCGTCTCTGCAGCCCACAACACCATCATTTGTGCGCGAGCTGGGAAA |
| | GACCTGGTCTCCAGCCTCACCTCGGGGCTGCTCACCATCGGGGATCGGTTTGGGGGTG |
| | CCTTGGATGCAGCAGCCAAGATGTTCAGTAAAGCCTTTGACAGTGGCATTATCCCCAT |
| | GGAGTTTGTGAACAAGATGAAGAAGGAAGGGAAGCTGATCATGGGCATTGGTCACCGA |
| | GTGAAGTCGATAAACAACCCAGACATGCGAGTGCAGATCCTCAAAGATTACGTCAGGC |
| | AGCACTTCCCTGCCACTCCTCTGCTCGATTATGCACTGGAAGTAGAGAAGATTACCAC |
| | CTCGAAGAAGCCAAATCTTATCCTGAATGTAGATGGTCTCATCGGAGTCGCATTTGTA |
| | GACATGCTTACAAACTGTGGGTCCTTTACTCGGGAGGAAGCTGATGAATATATTGACA |
| | TTGGAGCCCTCAATGGCATCTTTGTGCTGGGAACGAGTATGGGGTTCATTGGACACTA |
| | TCTTGATCAGAAGAGGCTGAAGCAGGGGCTGTATCGTCATCCGTGGGATGATATTTCA |
| | TATGTTCTTCCGGAACACATGAGCATGTAAGCGGCCGCTTTTTTCCTT |
| | ORF Start: at 2 ORF Stop: TAA at 3218 |
| | SEQ ID NO: 230 1072 aa MW at 117722.3kD |
| NOV15c, | QNSTMSAKAISEQTGKELLYKFICTTSAIQNRFKYARVTPDTDWARLLQDHPWLLSQN |
| CG142427-04 | |
| Protein Sequence | LVVKPDQLIKRRGKLGLVGVNLTLDGVKSWLKPRLGQEATVGKATGFLKNFLIEPFVP |
| | HSQAEEFYVCIYATREGDYVLFHHEGGVDVGDVDAKAQKLLVGVDEKLNPEDIKKHLL |
| | VHAPEDKKEILASFISGLFNFYEDLYFTYLEINPLVVTKDGVYVLDLAAKVDATADYI |
| | CKVKWGDIEFPPPFGREAYPEEAYIAGLDAKSGASLKLTLLNPKGRIWTMVAGGGASV |
| | VYSDTICDLGGVNELANYGEYSGAPSEQQTYDYAKTILSLMTREKHPDGKILIIGGSI |
| | ANFTNVAATFKGIVRAIRDYQGPLKEHEVTIFVRRGGPNYQEGLRVMGEVGKTTGIPT |
| | HVFGTETHTANFLLNASGSTSTPAPSRTASFSESRADEVAPAKKAKPANPQGKSTTLF |
| | SRHTKAIVWGMQTRAVQGMLDFDYVCSRDEPSVAANVYPFTGDHKQKFYWGHKEILIP |
| | VFKNMADAMRKHPEVDVLINFASLRSAYDSTMETTNYAQIRTIAIIAEGIPEALTRKL |
| | IKKADQKGVTIIGPATVGGIKPGCFKIGNTGGMLDNILASKLYRPGSVAYASRSGGMS |
| | NELNNIISRTTDGVYEGVAIGGDRYPGSTFMDHVLRYQDTPGVKMIVVLGEIGGTEEY |
| | KICRGIKEGRLTKPIVCWCIGTCATMFSSEVQFGHAGACANQASETAVAKNQALKEAG |
| | VFVPRSFDELGEIIQSVYEDLVANGVIVPAQEVPPPTVPMDYSWARELGLIRKPASFM |
| | TSICDERGQELIYAGMPITEVFKEEMGIGGVLGLLWFQKRLPKYSCQFIEMCLMVTAD |
| | HGPAVSGAHNTICARAGKDLVSSLTSGLLTIGDRFGGALDAAAKMFSKAFDSGIIPM |
| | EFVNKMKKEGKLIMGIGHRVKSINNPDMRVQILKDYVRQHFPATPLLDYALEVEKITT |
| | SKKPNLILNVDGLIGVAFVDMLRNCGSFTREEADEYIDIGALNGIFVLGRSMGFIGHY |
| | LDQKRLKQGLYRHPWDDISYVLPEIIMSM |
| | SEQ ID NO: 231 3307 bp |
| NOV15d, | CCAGAATTCCACCATGTCGGCCAAGGCAATTTCAGAGCAGACGGGCAAAGAACTCCTT |
| CG142427-02 | |
| DNA Sequence | TACAAGTTCATCTGTACCACCTCAGCCATCCAGAATCGGTTCAAGTATGCTCGGGTCA |
| | CTCCTGACACAGACTGGGCCCGCTTGCTGCAGGACCACCCCTGGCTGCTCAGCCAGAA |
| | CTTGGTAGTCAAGCCAGACCAGCTGATCAAACGTCCTGGAAAACTTGGTCTCGTTGGG |
| | GTCAACCTCACTCTGGATGGGGTCAAGTCCTGGCTGAAGCCACGGCTGGGACAGGAAG |
| | CCACAGTTGGCAAGGCCACAGGCTTCCTCAAGAACTTTCTGATCGAGCCCTTCGTCCC |
| | CCACAGTCAGGCTGAGGAGTTCTATGTCTGCATCTATGCCACCCGAGAAGGGGACTAC |
| | GTCCTGTTCCACCACGAGGGGGGTGTGGACGTGGGTGATGTGGACGCCAAGGCCCAGA |
| | AGCTGCTTGTTGGCGTGGATGAGAAACTGAATCCTGAGGACATCAAAAAACACCTGTT |
| | GGTCCACGCCCCTGAAGACAAGAAAGAAATTCTGGCCAGTTTTATCTCCGGCCTCTTC |
| | AATTTCTACGAGGACTTGTACTTCACCTACCTCGAGATCAATCCCCTTGTAGTGACCA |
| | AAGATGGAGTCTATGTCCTTGACTTGGCGGCCAAGGTGGACGCCACTGCCGACTACAT |
| | CTGCAAAGTGAAGTGGGGTGACATCGAGTTCCCTCCCCCCTTCGGGCGGGAGGCATAT |
| | CCAGAGGAAGCCTACATTGCAGACCTCGATGCCAAAAGTGGGGCAAGCCTGAAGCTGA |
| | CCTTGCTGAACCCCAAAGGGAGGATCTGGACCATGGTGGCCGGGGGTGGCGCCTCTGT |
| | CGTGTACAGCGATACCATCTGTGATCTAGGGGGTGTCAACGAGCTGGCAAACTATGGG |
| | GAGTACTCAGGCGCCCCCAGCGAGCAGCAGACCTATGACTATGCCAAGACTATCCTCT |
| | CCCTCATGACCCGAGAGAAGCACCCAGATGGCAAGATCCTCATCATTGGAGGCAGCAT |
| | CGCAAACTTCACCAACGTGGCTGCCACGTTCAAGGGCATCGTGAGAGCAATTCGAGAT |
| | TACCAGGGCCCCCTGAAGGAGCACGAAGTCACAATCTTTGTCCGAAGAGGTGGCCCCA |
| | ACTATCAGGAGGGCTTACGGGTGATGGGAGAAGTCGGGAAGACCACTGGGATCCCCAT |
| | CCATGTCTTTGGCACAGAGACTCACATGACGGCCATTGTGGGCATGGCCCTGGGCCAC |
| | CGGCCCATCCCCAACCAGCCACCCACAGCGGCCCACACTGCAAACTTCCTCCTCAACG |
| | CCAGCGGGAGCACATCGACGCCAGCCCCCAGCAGGACAGCATCTTTTTCTGAGTCCAG |
| | GGCCGATGAGGTGGCGCCTGCAAAGAAGGCCAAGCCTGCCATGCCACAAGGAAAGAGC |
| | ACCACCCTCTTCAGCCGCCACACCAAGGCCATTGTGTGGGGCATGCAGACCCGGGCCG |
| | TGCAAGGCATGCTGGACTTTGACTATGTCTGCTCCCGAGACGAGCCCTCAGTGGCTGC |
| | CATGGTCTACCCTTTCACTGGGGACCACAAGCAGAAGTTTTACTGOGGGCACAAAGAG |
| | ATCCTGATCCCTGTCTTCAAGAACATGGCTGATGCCATGAGGAAGCACCCGGAGGTAG |
| | ATGTGCTCATCAACTTTGCCTCTCTCCGCTCTGCCTATGACAGCACCATGGAGACCAT |
| | GAACTATGCCCAGATCCGGACCATCGCCATCATAGCTGAAGGCATCCCTGAGGCCCTC |
| | ACGAGAAAGCTGATCAAGAAGGCGGACCAGAAGGGAGTGACCATCATCGGACCTCCCA |
| | CTGTTGGAGGCATCAAGCCTGGGTGCTTTAAGATTGGCAACACAGGTGGGATGCTGGA |
| | CAACATCCTGGCCTCCAAACTGTACCGCCCAGGCAGCGTGGCCTATGTCTCACGTTCC |
| | GGAGGCATGTCCAACGAGCTCAACAATATCATCTCTCGGACCACGGATGGCGTCTATG |
| | AGGGCGTGGCCATTGGTGGGGACAGGTACCCGGGCTCCACATTCATGGATCATGTGTT |
| | ACGCTATCAGGACACTCCAGGAGTCAAAATGATTGTGGTTCTTGGAGAGATTGGGGGC |
| | ACTGAGGAATATAAGATTTGCCGGGGCATCAAGGAGGGCCGCCTCACTAAGCCCATCG |
| | TCTGCTGGTGCATCCGGACGTGTGCCACCATGTTCTCCTCTGAGGTCCAGTTTGGCCA |
| | TGCTGGAGCTTGTGCCAACCAGGCTTCTGAAACTGCAGTAGCCAAGAACCAGGCTTTG |
| | AAGGAAGCAGGAGTGTTTGTGCCCCGGAGCTTTGATGAGCTTGGAGAGATCATCCAGT |
| | CTGTATACGAAGATCTCGTGGCCAATGGAGTCATTGTACCTGCCCAGGAGGTGCCGCC |
| | CCCAACCGTGCCCATGGACTACTCCTGGGCCAGGGAGCTTGGTTTGATCCGCAAACCT |
| | GCCTCGTTCATGACCAGCATCTGCGATGAGCGAGGACAGGAGCTCATCTACGCGGGCA |
| | TGCCCATCACTGAGGTCTTCAAGGAAGAGATGGGCATTGGCGGGGTCCTCGGCCTCCT |
| | CTGGTTCCAGAAAAGGTTGCCTAAGTACTCTTGCCAGTTCATTGAGATGTGTCTGATG |
| | GTGACAGCTGATCACGGGCCAGCCGTCTCTGGAGCCCACAACACCATCATTTGTGCGC |
| | GAGCTGGGAAAGACCTGGTCTCCAGCCTCACCTCGGGGCTGCTCACCATCGGGGATCG |
| | GTTTGCGGGTGCCTTGGATGCAGCAGCCAAGATGTTCAGTAAAGCCTTTGACAGTGGC |
| | ATTATCCCCATGGAGTTTGTGAACAAGATGAAGAAGGAAGGGAAGCTGATCATGGGCA |
| | TTGGTCACCGAGTGAAGTCGATAAACAACCCAGACATGCGAGTGCAGATCCTCAAAGA |
| | TTACGTCAGGCAGCACTTCCCTGCCACTCCTCTGCTCGATTATGCACTGGAAGTAGAG |
| | AAGATTACCACCTCGAAGAAGCCAAATCTTATCCTGAATGTAGATGGTCTCATCGGAG |
| | TCGCATTTGTAGACATGCTTAGAAACTGTGGGTCCTTTACTCGGGAGGAAGCTGATGA |
| | ATATATTGACATTGGAGCCCTCAATGGCATCTTTGTGCTGGGAAGGAGTATGGGGTTC |
| | ATTGGACACTATCTTGATCAGAAGAGGCTGAAGCAGGGGCTGTATCGTCATCCGTGGG |
| | ATGATATTTCATATGTTCTTCCGGAACACATGAGCATGTAAGCGGCCGCTTTTTTCCT |
| | T |
| | ORF Start: at 2 ORF Stop: TAA at 3287 |
| | SEQ ID NO: 232 1095 aa MW at 120201.2kD |
| NOV15d, | QNSTMSAKAISEQTGKELLYKFICTTSAIQNRFKYARVTPDTDWARLLQDHPWLLSQN |
| CG142427-02 | |
| Protein Sequence | LVVKPDQLIKRRGKLGLVGVNLTLDGVKSWLKPRLGQEATVGKATGFLKNFLIEPFVP |
| | HSQAEEFYVCIYATREGDYVLFHHEGGVDVGDVDAKAQKLLVGVDEKLNPEDIKKHLL |
| | VHAPEDKKEILASFISGLFNFYEDLYFTYLEINPLVVTKDGVYVLDLAAKVDATADYI |
| | CKVKWGDIEFPPPFGREAYPEEAYIADLDAKSGASLKLTLLNPKGRIWTMVAGGGASV |
| | VYSDTICDLGGVNELANYGEYSGAPSEQQTYDYAKTILSLMTREKHPDGKILIIGGSI |
| | ANFTNVAATFKGIVRAIRDYQGPLKEHEVTIFVRRGGPNYQEGLRVMGEVGKTTGIPT |
| | HVFGTETHMTATVGMALGHRPIPNQPPTAAHTANFLLNASGSTSTPAPSRTASFSESR |
| | ADEVAPAKKAKPAMPQGKSTTLFSRHTKAIVWGMQTRAVQGMLDFDYVCSRDEPSVAA |
| | MVYPFTGDHKQKFYWGHKEILIPVFKNMADAMRKHPEVDVLINFASLRSAYDSTMETM |
| | NYAQIRTIATIAEGIPEALTRKLIKKADQKGVTIIGPATVGGIKPGCFKIGNTGGMLD |
| | NILASKLYRPGSVAYVSRSGGMSNELNNIISRTTDGVYEGVAIGGDRYPGSTFMDHVL |
| | RYQDTPGVKMIVVLGEIGGTEEYKICRGIKEGRLTKPIVCWCIGTCATMFSSEVQFGH |
| | AGACANQASETAVAKNQALKEAGVFVPRSFDELGEIIQSVYEDLVANGVIVPAQEVPP |
| | PTVPMDYSWARELGLIRKPASFMTSICDRRGQELIYAGMPITEVFKEEMGIGGVLGLL |
| | WFQKRLPKYSCQFIEMCLMVTADHGPAVSGAHNTIICARAGKDLVSSLTSGLLTIGDR |
| | FGGALDAAAKMFSKAFDSGIIPMEFVNKMKKEGKLIMGIGHRVKSINNPDMRVQILKD |
| | YVRQHFPATPLLDYALEVEKITTSKKPNLILNVDGLIGVAFVDMLRNCGSFTREEADE |
| | YIDIGALNGIFVLGRSMGFTGHYLDQKRLKQGLYRHPWDDISYVLPEHMSM |
| | SEQ ID NO: 233 2290 bp |
| NOV 15e, | CCAGAATTCCACCATGTCGGCCAAGGCAATTTCAGAGCAGACGGGCAAAGAACTCCTT |
| CG142427-03 | |
| DNA Sequence | TACAAGTTCATCTGTACCACCTCAGCCATCCAGAATCGGTTCAAGTATGCTCGGGTCA |
| | CTCCTGACACAGACTGGGCCCGCTTGCTGCAGGACCACCCCTGGCTGCTCAGCCAGAA |
| | CTTGGTAGTCAAGCCAGACCAGCTGATCAAACGTCCTGGAAAACTTGGTCTCGTTGGG |
| | GTCAACCTCACTCTGGATGGGGTCAAGTCCTGGCTGAAGCCACGGCTGGGACAGGAAG |
| | CCACAGTGAGTGGCATGGGGTCAAGATGAACGTGTGTGGTAACAGAAGCAAATATGG |
| | TCACCTTCAGGTTGGCAAGGCCACAGGCTTCCTCAAGAACTTTCTGATCGAGCCCTTC |
| | GTCCCCCACAGTCAGGCTGAGGAGTTCTATGTCTGCATCTATGCCACCCGAGAAGGGG |
| | ACTACGTCCTGTTCCACCACGAGGGGGGTGTGGACGTGGGTGATGTGGACGCCAAGGC |
| | CCAGAAGCTGCTTGTTGGCGTGGATGAGAAACTGAATCCTGAGGACATCAAAAAACAC |
| | CTGTTGGTCCACGCCCCTGAAGACAAGAAAGAAATTCTGGCCAGTTTTATCTCCGGCC |
| | TCTTCAATTTCTACGAGGACTTGTACTTCACCTACCTCGAGATCAATCCCCTTGTAGT |
| | GACCAAAGATGGAGTCTATGTCCTTGACTTGGCGGCCAAGGTGGACGCCACTGCCGAC |
| | TACATCTGCAAAGTGAAGTGGGGTGACATCGAGTTCCCTCCCCCCTTCGGGCGGGAGG |
| | CATATCCAGAGGAAGCCTACATTGCAGACCTCGACGCCAAAAGTGGGGCAAGCCTGAA |
| | GCTGACCTTGCTGAACCCCAAAGGGAGGATCTGGACCATGGTGGCCGGGGGTGGCGCC |
| | TCTGTCGTGTACAGCGATACCATCTGTGATCTAGGGGGTGTCAACGAGCTGGCAAACT |
| | ATGGGGAGTACTCAGGCGCCCCCAGCGAGCAGCACACCTATGACTATGCCAAGACTAT |
| | CCTCTCCCTCATGACCCGAGAGAAGCACCCAGATGGCAAGATCCTCATCATTGGAGGC |
| | AGCATCGCAAACTTCACCAACGTGGCTGCCACGTTCAAGGGCATCGTGAGAGCAATTC |
| | GAGATTACCAGGGCCCCCTGAAGGAGCACGAAGTCACAATCTTTGTCCGAAGAGGTGG |
| | CCCCAACTATCAGGAGGGCTTACGGGTGATGGGAGAAGTCGGGAAGACCACTGGGATC |
| | CCCATCCATGTCTTTGGCACAGAGACTCACATCACGGCCATTGTGGGCATGGCCCTGG |
| | GCCACCGGCCCATCCCCAACCAGCCACCCACACCGGCCCACACTGCAAACTTCCTCCT |
| | CAACGCCAGCGGGAGCACATCGACGCCAGCCCCCAGCAGGACAGCATCTTTTTCTGAG |
| | TCCAGGGCCGATGAGGTGGCGCCTGCAAAGAAGGCCAAGCCTGCCATGCCACAAGGAA |
| | AGAGCACCACCCTCTTCAGCCGCCACACCAAGGCCATTGTGTGGGGCATGCAGACCCG |
| | GGCCGTGCAAGGCATGCTGGACTTTGACTATGTCTGCTCCCGAGACGAGCCCTCAGTG |
| | GCTGCCATGGTCTACCCTTTCACTGGGGACCACAAGCAGAAGTTTTACTGGGGGCACA |
| | AAGAGATCCTGATCCCTGTCTTCAAGAACATGGCTGATGCCATGAGGAAGCACCCGGA |
| | GGTAGATGTGCTCATCAACTTTGCTTCTCTCCGCTCTGCCTTGGATGCAGCAGCCAAG |
| | ATGTTCAGTAAAGCCTTTGACAGTGGCATTATCCCCATGGAGTTTGTGAACAAGATGA |
| | AGAAGGAAGGGAAGCTGATCATGGGCATTGGTCACCGAGTGAAGTCGATAAACAACCC |
| | AGACATGCGAGTGCGGATCCTCAAAGATTACGTCAGGCAGCACTTCCCTGCCACTCCT |
| | CTGCTCGATTATGCACTGGAAGTAGAGAAGATTACCACCTCGAAGAAGCCAAATCTTA |
| | TCCTGAATGTAGATGGTCTCATCGGAGTCGCATTTGTAGACATGCTTAGAAACTGTGG |
| | GTCCTTTACTCGGGAGGAAGCTGATGAATATATTGACATTGGAGCCCTCAATGGCATC |
| | TTTGTGCTGGGAAGGAGTATGGGGTTCATTGGACACTATCTTGATCAGAAGAGGCTGA |
| | AGCAGGGGCTGTATCGTCATCCGTGGGATGATATTTCATATGTTCTTCCGGAACACAT |
| | GAGCATGTAAGCGGCCGCTTTTTTCCTT |
| | ORF Start: at 2 ORF Start: TAA at 2270 |
| | SEQ ID NO: 234 756 aa MW at 83890.7kD |
| NOV15e, | QNSTMSAKAISEQTGKELLYKFICTTSAIQNRFKYARVTPDTDWARLLQDHPWLLSQN |
| CG142427-03 | |
| Protein Sequence | LVVKPDQLIKRRGKJLGLVGVNLTLDGVKSWLKPRLGQEATVSGHGVKMNVCGNRSKYG |
| | HLQVGKATGFLKNFLIEPFVPHSQAEEFYVCIYATREGDYVLFHHEGGVDVGDVDAKA |
| | QKLLVGVDEKLNPEDIKKHLLVHAPEDKKEILASFISGLFNFYEDLYFTYLEINPLVV |
| | TKDGVYVLDLAAKVDATADYICKVKWGDIEFPPPFGREAYPEEAYIADLDAKSGASLK |
| | LTLLNPKGRIWTMVAGGGASXTVYSDTICDLGGVNELANYGEYSGAPSEQQTYDYAKTI |
| | LSLMTREKHPDGKTLIIGGSIANFThVAATFKGIVRAIRDYQGPLKEHEVTIFVRRGG |
| | PNYQEGLRVMGEVGKTTGIPIHVFGTETHMTAIVGMALGHRPIPNQPPTAAHTANFLL |
| | NASGSTSTPAPSRTASFSESRADEVAPAKKAKPANPQGKSTTLFSRHTKAIVWGMQTR |
| | AVQGMLDFDYVCSRDEPSVAAMVYPFTGDHKQKFYWGHKEILIPVFKNMADAMRKHPE |
| | VDVLINFASLRSALDAAAKMFSKAFDSGIIPMEFVNKMKKEGKLIMGIGHRVKSINNP |
| | DMRVRILKDYVRQHFPATPLLDYALEVEKITTSKKPNLILNVDGLTGVAFVDMLRNCC |
| | SFTREEADEYIDIGALNGIFVLGRSMGFIGHYLDQKRLKQGLYRHPWDDISYVLPEHM |
| | SEQ ID NO: 235 3317 bp |
| NOV15f | CCAGAATTCCACCATGTCGGCCAAGGCAATTTCAGAGCAGACGGGCAAAGAACTCCTT |
| 256388552 | |
| DNA Sequence | TACAAGTTCATCTGTACCACCTCAGCCATCCAGAATCGGTTCAAGTATGCTCGGGTCA |
| | CTCCTGACACAGACTGGGCCCGCTTGCTGCAGGACCACCCCTGGCTGCTCAGCCAGAA |
| | CTTGGTAGTCAAGCCAGACCAGCTGATCAAACGTCGTGGAAAACTTGGTCTCGTTGGG |
| | GTCAACCTCACTCTGGATGGGGTCAAGTCCTGGCTGAAGCCACGGCTGGGACAGGAAG |
| | CCACAGTTGGCAAGGCCACAGGCTTCCTCAAGAACTTTCTGATCGAGCCCTTCGTCCC |
| | CCACAGTCAGGCTGAGGAGTTCTATGTCTGCATCTATGCCACCCGAGAAGGGGACTAC |
| | GTCCTGTTCCACCACGAGGGGGGTGTGGACGTGGGTGATGTGGACGCCAAGGCCCAGA |
| | AGCTGCTTGTTGGCGTGGATGAGAAACTGAATCCTGAGGACATCAAAAAACACCTGTT |
| | GGTCCACGCCCCTGAAGACAAGAAAGAAATTCTGGCCAGTTTTATCTCCGGCCTCTTC |
| | AATTTCTACGAGGACTTGTACTTCACCTACCTCGAGATCAATCCCCTTGTAGTGACCA |
| | AAGATGGAGTCTATGTCCTTGACTTGGCGGCCAAGGTGGACGCCACTGCCGACTACAT |
| | CTGCAAAGTGAAGTGGGGTGACATCGAGTTCCCTCCCCCCTTCGGGCGGGAGGCATAT |
| | CCAGAGGAAGCCTACATTGCAGACCTCGATGCCAAAAGTGGGGCAAGCCTGAAGCTGA |
| | CCTTGCTGAACCCCAAAGGGAGGATCTGGACCATGGTGGCCGGGGGTGGCGCCTCTGT |
| | CGTGTACAGCGATACCATCTGTGATCTAGGGGGTGTCAACGAGCTGGCAAACTATGGG |
| | GAGTACTCAGGCGCCCCCAGCGAGCAGCAGACCTATGATTATGCCAAGACTATCCTCT |
| | CCCTCATGACCCGAGAGAAGCACCCAGATGGCAAGATCCTCATCATTGGAGGCAGCAT |
| | CGCAAACTTCACCAACGTGGCTGCCACGTTCAAGGGCATCGTGAGAGCAATTCGAGAT |
| | TACCAGGGCCCCCTGAAGGAGCACGAAGTCACAATCTTTGTCCGAAGAGGTGGCCCCA |
| | ACTATCAGGAGGGCTTACGGGTGATGGGAGAAGTCGGGAAGACCACTGGGATCCCCAT |
| | CCATGTCTTTGGCACAGAGACTCACATGACGGCCATTGTGGGCATGGCCCTGGGCCAC |
| | CGGCCCATCCCCAACCAGCCACCCACAGCGGCCCACACTGCAAACTTCCTCCTCAACG |
| | CCAGCGGGAGCACATCGACGCCAGCCCCCAGCAGGACAGCATCTTTTTCTGAGTCCAG |
| | GGCCGATGAGGTGGCGCCTGCAAAGAAGGCCAAGCCTGCCATGCCACAAGATTCAGTC |
| | CCAAGTCCAAGATCCCTGCAAGGAAAGAGCACCACCCTCTTCAGCCGCCACACCAAGG |
| | CCATTGTGTGGGGCATGCAGACCCGGGCCGTGCAAGGCATGCTGGACTTTGACTATGT |
| | CTGCTCCCGAGACGAGCCCTCAGTGGCTGCCATGGTCTACCCTTTCACTGGGGACCAC |
| | AAGCAGAAGTTTTACTGGGGGCACAAAGAGATCCTGATCCCTGTCTTCAAGAACATGG |
| | CTGATGCCATGAGGAAGCACCCGGAGGTAGATGTGCTCATCAACTTTGCCTCTCTCCG |
| | CTCTGCCTATGACAGCACCATGGAGACCATGAACTATGCCCAGATCCGGACCATCGCC |
| | ATCATAGCTGAAGGCATCCCTGAGGCCCTCACGAGAAAGCTGATCAAGAAGGCGGACC |
| | AGAAGGGAGTGACCATCATCGGACCTGCCACTGTTGGAGGCATCAAGCCTGGGTGCTT |
| | TAAGATTGGCAACACAGGTGGGATGCTGGACAACATCCTGGCCTCCAAACTGTACCGC |
| | CCAGGCAGCGTGGCCTATGTCTCACGTTCCGGAGGCATGTCCAACGAGCTCAACAATA |
| | TCATCTCTCGGACCACGGATGGCGTCTATGAGGGCGTGGCCATTGGTGGGGACAGGTA |
| | CCCGGGCTCCACATTCATGGATCATGTGTTACGCTATCAGGACACTCCAGGAGTCAAA |
| | ATGATTGTGGTTCTTGGAGAGATTGGGGGCACTGAGGAATATAAGATTTGCCGGGGCA |
| | TCAAGGACGGCCGCCTCACTAAGCCCATCGTCTGCTGGTGCATCGGGACGTGTGCCAC |
| | CATGTTCTCCTCTGAGGTCCAGTTTGGCCATGCTGGAGCTTGTGCCAACCAGGCTTCT |
| | GAAACTGCAGTAGCCAAGAACCAGGCTTTGAAGGAAGCAGGAGTGTTTGTGCCCCGGA |
| | GCTTTGATGAGCTTGGAGAGATCATCCAGTCTGTATACGAAGATCTCGTGGCCAATGG |
| | AGTCATTGTACCTGCCCAGGAGGTGCCGCCCCCAACCGTGCCCATGGACTACTCCTGG |
| | GCCAGGGAGCTTGGTTTGATCCGCAAACCTGCCTCGTTCATGACCAGCATCTGCGATG |
| | AGCGAGGACAGGAGCTCATCTACGCGGGCATGCCCATCACTGAGGTCTTCAAGGAAGA |
| | GATGGGCATTGGCCGGGTCCTCGGCCTCCTCTGGTTCCAGAAAAGGTTGCCTAAGTAC |
| | TCTTGCCAGTTCATTGAGATGTGTCTGATGGTGACAGCTGATCACGGGCCAGCCGTCT |
| | CTGGAGCCCACAACACCATCATTTGTGCGCGAGCTGGGAAAGACCTGGTCTCCAGCCT |
| | CACCTCGGGGCTGCTCACCATCGGGGATCGGTTTGGGGGTGCCTTGGATGCAGCAGCC |
| | AAGATGTTCAGTAAAGCCTTTGACAGTGGCATTATCCCCATGGAGTTTGTGAACAAGA |
| | TGAAGAAGGAAGGGAAGCTGATCATGGGCATTGGTCACCGAGTGAAGTCGATAAACAA |
| | CCCAGACATGCGAGTGCAGATCCTCAAAGATTACGTCAGGCAGCACTTCCCTGCCACT |
| | CCTCTGCTCGATTATGCACTGGAAGTAGAGAAGATTACCACCTCGAAGAAGCCAAATC |
| | TTATCCTGAATGTAGATGGTCTCATCGGAGTCGCATTTGTAGACATGCTTAGAAACTG |
| | TGGGTCCTTTACTCGGGAGGAAGCTGATGAATATATTGACATTGGAGCCCTCAATGGC |
| | ATCTTTGTGCTGGGAAGGAGTATGGGGTTCATTGGACACTATCTTGATCAGAAGAGGC |
| | TGAAGCAGGGGCTGTATCGTCATCCGTGGGATGATATTTCATATGTTCTTCCGGAACA |
| | CATGAGCATGT |
| | ORF Start: at 2 ORF Stop: end of sequence |
| | SEQ ID NO: 236 1106 aa MW at 121268.4kD |
| NOV15f, | QNSTMSAKAISEQTGKELLYKFICTTSAILQNRFKYARVTPDTDWARLLQDHPWLLSQN |
| 256388552 | |
| Protein Sequence | LVVKPDQLIKRRGKLGLVGVNLTLDGVKSWLKPRLGQEATVGKATGFLKNFLIEPFVP |
| | HSQAEEFYVCIYATREGDYVLFHHEGGVDVGDVDAKAQKLLVGVDEKLNPEDIKKHLL |
| | VHAPEDKKETLASFISGLFNFYEDLYFTYLEINPLVVTKDGVYVLDLAAKVDATADYI |
| | CKVKWGDIEFPPPFGREAYPEEAYIADLDAKSGASLKLTLLNPKCRIWTMVAGGGASV |
| | VYSDTICDLGGVNELANYGEYSGAPSEQQTYDYAKTILSLMTREKHPDGKILIIGGSI |
| | ANFTNVAATFKGIVRAIRDYQGPLKEHEVTIFVRRGGPNYQEGLRVMGEVGKTTGIPT |
| | HVFGTETHMTAIVGMALGHRPIPNQPPTAAHTANFLLNASGSTSTPAPSRTASFSESR |
| | ADEVAPAKKAKPAMPQDSVPSPRSLQGKSTTLFSRHTKAIVWGMQTRAVQGMLDFDYV |
| | CSRDEPSVAAMVYPFTGDHKQKFYWGHKEILIPVFKNMADAMRKHPEVDVLINFASLR |
| | SAYDSTMETMNYAQIRTIAIIAEGIPEALTRKLIKKADQKGVTIIGPATVGGIKPGCF |
| | KIGNTGGMLDNILASKLYRPGSVAYVSRSGGMSNELNNIISRTTDGVYEGVAIGGDRY |
| | PGSTFMDHVLRYQDTPGVKMIVVLGEIGGTEEYKICRGIKEGRLTKPIVCWCIGTCAT |
| | MFSSEVQFGHAGACANQASETAVAKNQALKEAGVFVPRSFDELGEIIQSVYEDLVANG |
| | VIVPAQEVPPPTVPMDYSWARELGLIRKPASFMTSICDERGQELIYAGMPITEVFKEE |
| | MGIGGVLGLLWFQKRLPKYSCQFIEMCLMVTADHGPAVSGAHNTIICARAGKDLVSSL |
| | TSGLLTIGDRFGGALDAAAKMFSKAFDSGIIPMEFVNKMKKEGKLIMGIGHRVKSINN |
| | PDMRVQILKDYVRQHFPATPLLDYALEVEKITTSKKPNLILNVDGLIGVAFVDMLRNC |
| | GSFTREEADEYIDIGALNGIFVLGRSMGFIGHYLDQKRLKQGLYRHPWDDISYVLPEH |
| | SEQ ID NO: 237 3307 bp |
| NOV15g, | CCAGAATTCCACCATGTCGGCCAAGGCAATTTCAGAGCAGACGGGCAAAGAACTCCTT |
| 256420210 | |
| DNA Sequence | TACAAGTTCATCTGTACCACCTCAGCCATCCAGAATCGGTTCAAGTATGCTCGGGTCA |
| | CTCCTGACACAGACTGGGCCCGCTTGCTGCAGGACCACCCCTGGCTGCTCAGCCAGAA |
| | CTTGGTAGTCAAGCCAGACCAGCTGATCAAACGTCGTGGAAAACTTGGTCTCGTTGGG |
| | GTCAACCTCACTCTGGATGGGGTCAAGTCCTGGCTGAAGCCACGGCTGGGACAGGAAG |
| | CCACAGTTGGCAAGGCCACAGGCTTCCTCAAGAACTTTCTGATCGAGCCCTTCGTCCC |
| | CCACAGTCAGGCTGAGGAGTTCTATGTCTGCATCTATGCCACCCGAGAAGGGGACTAC |
| | GTCCTGTTCCACCACGAGGGGGGTGTGGACGTGGGTGATGTGGACGCCAAGGCCCAGA |
| | AGCTGCTTGTTGGCGTGGATGAGAAACTGAATCCTGAGGACATCAAAAAACACCTGTT |
| | GGTCCACGCCCCTGAAGACAAGAAAGAAATTCTGGCCAGTTTTATCTCCGGCCTCTTC |
| | AATTTCTACGAGGACTTGTACTTCACCTACCTCGAGATCAATCCCCTTGTAGTGACCA |
| | AAGATGGAGTCTATGTCCTTGACTTGGCGGCCAAGGTGGACGCCACTGCCGACTACAT |
| | CTGCAAAGTGAAGTGGGGTGACATCGAGTTCCCTCCCCCCTTCGGCCGGGAGGCATAT |
| | CCAGAGGAAGCCTACATTGCAGACCTCGATGCCAAAAGTGGGGCAAGCCTGAAGCTGA |
| | CCTTCCTGAACCCCAAAGGGAGGATCTGGACCATGGTGGCCGGGGGTGGCGCCTCTCT |
| | CGTGTACAGCGATACCATCTGTGATCTAGGGGGTGTCAACGAGCTGGCAAACTATGGG |
| | GAGTACTCAGGCGCCCCCAGCGAGCAGCAGACCTATGACTATGCCAAGACTATCCTCT |
| | CCCTCATGACCCGAGAGAAGCACCCAGATGGCAAGATCCTCATCATTGGAGGCAGCAT |
| | CGCAAACTTCACCAACGTGGCTGCCACGTTCAAGGGCATCGTGAGAGCAATTCGAGAT |
| | TACCAGGGCCCCCTGAAGGAGCACGAAGTCACAATCTTTGTCCGAAGAGGTGGCCCCA |
| | ACTATCAGGAGGGCTTACGGGTGATGGGAGAAGTCGGGAAGACCACTGGGATCCCCAT |
| | CCATGTCTTTGGCACAGAGACTCACATGACGGCCATTGTGGGCATGGCCCTGGGCCAC |
| | CGGCCCATCCCCAACCAGCCACCCACAGCGGCCCACACTGCAAACTTCCTCCTCAACG |
| | CCAGCGGGAGCACATCGACGCCAGCCCCCAGCAGGACAGCATCTTTTTCTGAGTCCAG |
| | GGCCGATGAGGTGGCGCCTGCAAAGAAGGCCAAGCCTGCCATGCCACAAGGAAAGAGC |
| | ACCACCCTCTTCAGCCGCCACACCAAGGCCATTGTGTGGGGCATGCAGACCCGGGCCG |
| | TGCAAGGCATGCTGGACTTTGACTATGTCTGCTCCCGAGACGAGCCCTCAGTGGCTGC |
| | CATGGTCTACCCTTTCACTGGGGACCACAAGCAGAAGTTTTACTGGGGGCACAAAGAG |
| | ATCCTGATCCCTGTCTTCAAGAACATGGCTGATGCCATGAGGAAGCACCCGGAGGTAG |
| | ATGTGCTCATCAACTTTGCCTCTCTCCGCTCTGCCTATGACAGCACCATGGAGACCAT |
| | GAACTATGCCCAGATCCGGACCATCGCCATCATAGCTGAAGGCATCCCTGAGGCCCTC |
| | ACGAGAAAGCTGATCAAGAAGGCGGACCAGAAGGGAGTGACCATCATCGGACCTGCCA |
| | CTGTTGAGGCATCAAGCCTGGGTGCTTTAAGATTGGCAACACAGGTGGGATGCTGGA |
| | CAACATCCTGGCCTCCAAACTGTACCGCCCAGGCAGCGTGGCCTATGTCTCACGTTCC |
| | GGAGGCATGTCCAACGAGCTCAACAATATCATCTCTCGGACCACGGATGGCGTCTATG |
| | AGGGCGTGGCCATTGGTGGGGACAGGTACCCGGGCTCCACATTCATGCATCATGTGTT |
| | ACGCTATCAGGACACTCCACGAGTCAAAATGATTGTGGTTCTTGGAGAGATTGGGGGO |
| | ACTGAGGAATATAAGATTTCCCGGGGCATCAAGGAGGGCCGCCTCACTAAGCCCATCG |
| | TCTGCTGGTGCATCGGGACGTGTGCCACCATGTTCTCCTCTGAGGTCCAGTTTGGCCA |
| | TGCTGGAGCTTGTGCCAACCAGGCTTCTGAAACTGCAGTAGCCAAGAACCAGGCTTTG |
| | AAGGAAGCAGGAGTGTTTGTGCCCCGGAGCTTTGATGAGCTTGGAGAGATCATCCAGT |
| | CTGTATACGAAGATCTCGTGGCCAATGGAGTCATTGTACCTGCCCAGGAGGTGCCGCC |
| | CCCAACCGTGCCCATGGACTACTCCTGGGCCAGGGAGCTTGGTTTGATCCGCAAACCT |
| | GCCTCGTTCATGACCAGCATCTGCGATGAGCGAGGACAGGAGCTCATCTACGCGGGCA |
| | TGCCCATCACTGAGGTCTTCAAGGAAGAGATGGGCATTGGCGGGGTCCTCGGCCTCCT |
| | CTGGTTCCAGAAAAGGTTGCCTAAGTACTCTTCCCAGTTCATTGAGATGTGTCTGATG |
| | GTGACAGCTGATCACGGGCCAGCCGTCTCTGGAGCCCACAACACCATCATTTGTGCGC |
| | GAGCTGGGAAAGACCTGGTCTCCAGCCTCACCTCGGGGCTGCTCACCATCGGGGATCG |
| | GTTTGGGGGTGCCTTGGATGCAGCAGCCAAGATGTTCAGTAAAGCCTTTGACAGTGGC |
| | ATTATCCCCATGGAGTTTGTGAACAAGATCAACAAGGAAGCGAAGCTGATCATGGGCA |
| | TTGGTCACCGAGTGAAGTCGATAAACAACCCAGACATGCGAGTGCAGATCCTCAAAGA |
| | TTACGTCAGGCAGCACTTCCCTGCCACTCCTCTGCTCGATTATGCACTGGAAGTAGAG |
| | AAGATTACCACCTCGAAGAAGCCAAATCTTATCCTGAATGTAGATGGTCTCATCGGAG |
| | TCGCATTTGTAGACATGCTTAGAAACTGTGGGTCCTTTACTCGGGAGGAAGCTGATGA |
| | ATATATTGACATTGGAGCCCTCAATGGCATCTTTGTGCTGGGAAGGAGTATGGGGTTC |
| | ATTGGACACTATCTTGATCAGAAGAGGCTGAAGCAGGGGCTGTATCGTCATCCGTGGG |
| | ATGATATTTCATATGTTCTTCCGGAACACATGAGCATGTAAGCGGCCGCTTTTTTCCT |
| | ORF Start: at 2 ORF Stop: TAA at 3287 |
| | SEQ ID NO: 238 1095 aa MW at 120201.2kD |
| NOV15g, | QNSTMSAKAISEQTGKELLYKFICTTSAIQNRFKYARVTPDTDWARLLQDNPWLLSQN |
| 256420210 | |
| Protein Sequence | LVVKPDQLIKRRGKLGLVGVNLTLDGVKSWLKPRLGQEATVGKATGFLKNFLIEPFVP |
| | HSQAEEFYVCIYATREGDYVLFHHEGGVDVGDVDAKAQKLLVGVDEKLNPEDIKKHLL |
| | VHAPEDKKEILASFISGLFNFYEDLYFTYLEINPLVVTKDGVYVLDLAAKVDATADYI |
| | CKVKWGDIEFPPPFGREAYPEEAYIADLDAKSGASLKLTLLNPKGRIWTMVAGGGASV |
| | VYSDTICDLGGVNELANYGEYSGAPSEQQTYDYAKTILSLMTREKHPDGKILIIGGSI |
| | ANFTNVAATFKGIVPAIRDYQGPLKEHEVTIFVRRGGPNYQEGLRVMGEVGKTTGIPI |
| | HVFGTETHMTAIVGMALGHRPIPNQPPTAAHTANFLLNASGSTSTPAPSRTASFSESR |
| | ADEVAPAKKAKPAMPQGKSTTLFSRHTKAIVWGMQTRAVQGMLDFDYVCSRDEPSVAA |
| | MVYPFTGDHKQKFYWGHKEILIPVFKNMADAMRKHPEVDVLINFASLRSAYDSTMETM |
| | NYAQIRTIAIIAEGIPEALTRKLIKKADQKGVTIIGPATVGGIKPGCFKIGNTGGMLD |
| | NILASKLYRPGSVAYVSRSGGMSNELNNIISRTTDGVYEGVAIGGDRYPGSTFMDHVL |
| | RYQDTPGVKMIVVLGEIGGTEEYKICRGIKEGRLTKPIVCWCIGTCATMFSSEVQFCH |
| | AGACANQASETAVAKNQALKEAGVFVPRSFDELGEIIQSVYEDLVANGVIVPAQEVPP |
| | PTVPMDYSWARELGLIRKPASFMTSICDERGQELIYAGMPITEVFKEEMGIGGVLGLL |
| | WFQKRLPKYSCQFIEMCLMVTADHGPAVSGAHNTIICARAGKDLVSSLTSGLLTIGDR |
| | FGGALDAAAKMFSKAFDSGIIPMEFVNKMKKEGKLIMGIGHRVKSINNPDMRVQILKD |
| | YVRQHFPATPLLDYALEvEKITTSKKPNLILNVDGLIGVAFVDMLRNCGSFTREEADE |
| | YIDIGALNGIFVLGRSMCFIGHYLDQKRLKQGLYRHPWDDISYVLPEHMSM |
| | SEQ ID NO: 239 2290 bp |
| NOV15h, | CCAGAATTCCACCATGTCGGCCAAGGCAATTTCAGAGCAGACGGGCAAAGAACTCCTT |
| 256202925 | |
| DNA Sequence | TACAAGTTCATCTGTACCACCTCAGCCATCCAGAATCGGTTCAAGTATGCTCGGGTCA |
| | CTCCTGACACAGACTGGGCCCGCTTGCTGCAGGACCACCCCTGGCTGCTCAGCCAGAA |
| | CTTGGTAGTCAAGCCAGACCAGCTGATCAAACGTCGTGGAAAACTTGGTCTCGTTGGG |
| | GTCAACCTCACTCTGGATGGGGTCAAGTCCTGGCTGAAGCCACGGCTGGGACAGGAAG |
| | CCACAGTGAGTGGGCATGGGGTCAAGATGAACGTGTGTGGTAACAGAAGCAAATATGG |
| | TCACCTTCAGGTTGGCAAGGCCACAGGCTTCCTCAAGAACTTTCTGATCGAGCCCTTC |
| | GTCCCCCACAGTCAGGCTGAGGAGTTCTATGTCTGCATCTATGCCACCCGAGAAGGGG |
| | ACTACGTCCTGTTCCACCACGAGGGGGGTGTGGACGTGGGTGATGTGGACGCCAAGGC |
| | CCAGAAGCTGCTTGTTGGCGTGGATGAGAAACTGAATCCTGAGGACATCAAAAAACAC |
| | CTGTTGGTCCACGCCCCTGAAGACAAGAAAGAAATTCTGGCCAGTTTTATCTCCGGCC |
| | TCTTCAATTTCTACGAGGACTTGTACTTCACCTACCTCGAGATCAATCCCCTTGTAGT |
| | GACCAAAGATGGAGTCTATGTCCTTGACTTGGCGGCCAAGGTGGACGCCACTGCCGAC |
| | TACATCTGCAAAGTGAAGTGGGGTGACATCGAGTTCCCTCCCCCCTTCGGGCGGGAGG |
| | CATATCCAGAGGAAGCCTACATTGCAGACCTCGACGCCAAAAGTGGGGCAAGCCTGAA |
| | GCTGACCTTGCTGAACCCCAAAGGGAGGATCTGGACCATGGTGGCCGGGGGTGGCGCC |
| | TCTGTCGTGTACAGCGATACCATCTGTGATCTAGGGGGTGTCAACGAGCTGGCAAACT |
| | ATGGGGAGTACTCAGGCGCCCCCAGCGAGCAGCAGACCTATGACTATGCCAAGACTAT |
| | CCTCTCCCTCATGACCCGAGAGAAGCACCCAGATGGCAAGATCCTCATCATTGGAGGC |
| | AGCATCGCAAACTTCACCAACGTGGCTGCCACGTTCAAGGGCATCGTGAGAGCAATTC |
| | GAGATTACCAGGGCCCCCTGAAGGAGCACGAAGTCACAATCTTTGTCCGAAGAGGTGG |
| | CCCCAACTATCAGGAGGGCTTACGGGTGATGGGAGAAGTCGGGAAGACCACTGGGATC |
| | CCCATCCATGTCTTTGGCACAGAGACTCACATGACGGCCATTGTGGGCATGGCCCTGG |
| | GCCACCGGCCCATCCCCAACCAGCCACCCACAGCGGCCCACACTGCAAACTTCCTCCT |
| | CAACGCCAGCGGGAGCACATCGACGCCAGCCCCCAGCAGGACAGCATCTTTTTCTGAG |
| | TCCAGGGCCGATCAGGTGGCGCCTGCAAAGAAGGCCAAGCCTGCCATGCCACAAGGAA |
| | AGAGCACCACCCTCTTCAGCCGCCACACCAAGGCCATTGTGTGGGGCATGCAGACCCG |
| | GGCCGTGCAAGGCATGCTGGACTTTGACTATGTCTGCTCCCGAGACGAGCCCTCAGTG |
| | GCTGCCATGGTCTACCCTTTCACTGGGGACCACAAGCAGAAGTTTTACTGGGGGCACA |
| | AAGAGATCCTGATCCCTGTCTTCAAGAACATGGCTGATGCCATGAGGAAGCACCCGGA |
| | GGTAGATGTGCTCATCAACTTTGCTTCTCTCCGCTCTGCCTTGGATGCAGCAGCCAAG |
| | ATGTTCAGTAAAGCCTTTGACAGTGGCATTATCCCCATGGAGTTTGTGAACAAGATGA |
| | AGAAGGAAGGGAAGCTGATCATGGGCATTGGTCACCGAGTGAAGTCGATAAACAACCC |
| | AGACATGCGAGTGCGGATCCTCAAAGATTACGTCAGGCAGCACTTCCCTGCCACTCCT |
| | CTGCTCGATTATGCACTGGAAGTAGAGAACATTACCACCTCGAAGAAGCCAAATCTTA |
| | TCCTGAATGTAGATGGTCTCATCGGAGTCGCATTTGTAGACATGCTTAGAAACTGTGG |
| | GTCCTTTACTCGGGAGGAAGCTGATGAATATATTGACATTGGAGCCCTCAATGGCATC |
| | TTTGTGCTGGGAAGGAGTATGGGGTTCATTGGACACTATCTTGATCAGAAGAGGCTGA |
| | AGCAGGGGCTGTATCGTCATCCGTGGGATGATATTTCATATGTTCTTCCGGAACACAT |
| | GAGCATGTAAGCGGCCGCTTTTTTCCTT |
| | ORF Start: at 2 ORF Stop: TAA at 2270 |
| | SEQ ID NO: 240 756 aa MW at 83890.7kD |
| NOV15h, | QNSTMSAKAISEQTGKELLYKFICTTSAIQNRFKYARVTPDTDWARLLQDHPWLLSQN |
| 256202925 | |
| Protein Sequence | LVVKPDQLIKRRGKLGLVGVNLTLDGVKSWLKPRLGQEATVSGHGVKMNVCGNRSKYG |
| | HLQVGKATGFLKNFLIEPFVPHSQAEEFYVCIYATREGDYVLFHHEGGVDVGDVDAKA |
| | QKLLVGVDEKLNPEDIKKHLLVHAPEDKKEILASFISGLFNFYEDLYFTYLEINPLVV |
| | TKDGVYVLDLAAKVDATADYICKVKWGDIEFPPPFGREAYPEEAYIADLDAKSGASLK |
| | LTLLNPKGRIWTMVAGGGASVVYSDTICDLGGVNELANYGEYSGAPSEQQTYDYAKTI |
| | LSLMTREKHPDGKILIIGGSIANFTNVAATFKGIVRAIRDYQGPLKEHEVTIFVRRGG |
| | PNYQEGLRVMGEVGKTTGIPIHVFGTETHMTAIVGMALGHRPIPNQPPTAAHTANFLL |
| | NASGSTSTPAPSRTASFSESRADEVAPAKKAKPAIVTPQGKSTTLFSRHTKAIVWGMQTR |
| | AVQGMLDFDYVCSRDEPSVAAMVYPFTGDHKQKFYWGHKEILIPVFKNMADANRKHPE |
| | VDVLINFASLRSALDAAAKMFSKAFDSGIIPMEFVNKMKKEGKLIMGIGHRVKSINNP |
| | DMRVRILKDYVRQHFPATPLLDyALEVEKITTSKKPNLILNVDGLIGVAFVDMLRNCG |
| | SFTREEADEYIDIGALNGIFVLGRSMGFIGHYLDQKRLKQGLYRHPWDDISYXTLPEHM |
| | SM |
| | SEQ ID NO: 241 3310 bp |
| NOV5j, | CACCATGTCGGCCAAGGCAATTTCAGAGCAGACGGGCAAAGAACTCCTTTACAAGTTC |
| 259856081 | |
| DNA Sequence | ATCTGTACCACCTCAGCCATCCAGAATCGGTTCAAGTATGCTCGGGTCACTCCTGACA |
| | CAGACTGGGCCCGCTTGCTGCAGGACCACCCCTGGCTGCTCAGCCAGAACTTGGTAGT |
| | CAAGCCAGACCAGCTGATCAAACGTCGTGGAAAACTTGGTCTCGTTGGGGTCAACCTC |
| | ACTCTGGATGGGGTCAAGTCCTGGCTGAAGCCACGGCTGGGACAGGAAGCCACAGTTG |
| | GCAAGGCCACAGGCTTCCTCAAGAACTTTCTGATCGAGCCCTTCGTCCCCCACAGTCA |
| | GGCTGAGGAGTTCTATGTCTGCATCTATGCCACCCGAGAAGGGGACTACGTCCTGTTC |
| | CACCACGAGGGGGGTGTGGACGTGGGTGATGTGGACGCCAAGGCCCAGAAGCTGCTTG |
| | TTGGCGTGGATGAGAAACTGAATCCTGAGGACATCAAAAAACACCTGTTGGTCCACGC |
| | CCCTGAAGACAAGAAAGAAATTCTGGCCAGTTTTATCTCCGGCCTCTTCAATTTCTAC |
| | GAGGACTTGTACTTCACCTACCTCGAGATCAATCCCCTTGTAGTGACCAAAGATGGAG |
| | TCTATGTCCTTGACTTGGCGGCCAAGGTGGACGCCACTGCCGACTACATCTGCAAAGT |
| | GAAGTCGGGTGACATCGAGTTCCCTCCCCCCTTCGGGCGGGAGGCATATCCAGAGGAA |
| | GCCTACATTGCAGACCTCGATGCCAAAAGTGGGGCAAGCCTGAAGCTGACCTTGCTGA |
| | ACCCCAAAGGGAGGATCTGGACCATGGTGGCCGGGGGTGGCGCCTCTGTCGTGTACAG |
| | CGATACCATCTGTGATCTAGGGGGTGTCAACGAGCTGGCAAACTATGGGGAGTACTCA |
| | GGCGCCCCCAGCGAGCAGCAGACCTATGATTATGCCAAGACTATCCTCTCCCTCATGA |
| | CCCGAGAGAAGCACCCAGATGGCAAGATCCTCATCATTGGAGCCAGCATCGCAAACTT |
| | CACCAACGTGGCTGCCACGTTCAAGGGCATCGTGAGAGCAATTCGAGATTACCAGGGC |
| | CCCCTGAAGGAGCACGAAGTCACAATCTTTGTCCGAAGAGGTGGCCCCAACTATCAGG |
| | AGGGCTTACGGGTGATGGGAGAAGTCGGGAAGACCACTGGGATCCCCATCCATGTCTT |
| | TGGCACAGAGACTCACATGACGGCCATTGTGGGCATGGCCCTGGGCCACCGGCCCATC |
| | CCCAACCAGCCACCCACAGCGGCCCACACTGCAAACTTCCTCCTCAACGCCAGCGGGA |
| | GCACATCGACGCCAGCCCCCAGCAGGACAGCATCTTTTTCTGAGTCCAGGGCCGATGA |
| | GGTGGCGCCTGCAAAGAAGGCCAAGCCTGCCATGCCACAAGATTCAGTCCCAAGTCCA |
| | AGATCCCTGCAAGGAAAGAGCACCACCCTCTTCAGCCGCCACACCAAGGCCATTGTGT |
| | GGGGCATGCAGACCCCGGCCCTGCAAGGCATGCTGGACTTTGACTATGTCTGCTCCCC |
| | AGACGAGCCCTCAGTGGCTGCCATGGTCTACCCTTTCACTGGGGACCACAAGCAGAAG |
| | TTTTACTGGGGGCACAAAGAGATCCTGATCCCTGTCTTCAAGAACATGGCTGATGCCA |
| | TGAGGAAGCACCCGGAGGTAGATGTGCTCATCAACTTTGCCTCTCTCCGCTCTGCCTA |
| | TGACAGCACCATGGAGACCATGAACTATGCCCAGATCCCGACCATCGCCATCATAGCT |
| | GAAGGCATCCCTGAGGCCCTCACCAGAAAGCTGATCAAGAAGCCGGACCAGAAGGGAG |
| | TGACCATCATCGGACCTGCCACTGTTGGAGGCATCAAGCCTGGGTGCTTTAAGATTGG |
| | CAACACAGGTGGGATGCTGGACAACATCCTGGCCTCCAAACTGTACCGCCCAGGCAGC |
| | GTGGCCTATGTCTCACGTTCCGGAGGCATGTCCAACGAGCTCAACAATATCATCTCTC |
| | GGACCACGGATGGCGTCTATCAGGGCGTGGCCATTGGTGGGGACAGGTACCCCGGCTC |
| | CACATTCATGGATCATGTGTTACGCTATCAGGACACTCCAGGAGTCAAAATGATTGTG |
| | GTTCTTGGAGAGATTGGGGGCACTGAGGAATATAAGATTTGCCGCGGCATCAAGGAGG |
| | GCCGCCTCACTAAGCCCATCGTCTGCTGGTGCATCGGGACGTGTGCCACCATGTTCTC |
| | CTCTGAGGTCCAGTTTGGCCATGCTGGAGCTTGTGCCAACCAGGCTTCTGAAACTGCA |
| | GTAGCCAAGAACCAGGCTTTGAAGGAAGCAGGAGTGTTTGTGCCCCGGAGCTTTGATG |
| | AGCTTGGAGAGATCATCCAGTCTGTATACGAAGATCTCGTGGCCAATGGAGTCATTGT |
| | ACCTGCCCAGGAGGTGCCGCCCCCAACCGTGCCCATGGACTACTCCTGGGCCAGGGAG |
| | CTTGGTTTGATCCGCAAACCTGCCTCGTTCATGACCAGCATCTGCGATGAGCGAGGAC |
| | AGGAGCTCATCTACGCGGGCATGCCCATCACTGAGGTCTTCAAGGAAGAGATGGGCAT |
| | TGGCGGGGTCCTCGGCCTCCTCTGGTTCCAGAAAAGGTTGCCTAAGTACTCTTGCCAG |
| | TTCATTGAGATGTGTCTGATGGTGACAGCTGATCACGGGCCAGCCGTCTCTGGAGCCC |
| | ACAACACCATCATTTGTGCGCGAGCTGGGAAAGACCTGGTCTCCAGCCTCACCTCGGG |
| | GCTGCTCACCATCGGGGATCGGTTTGGGGGTGCCTTGGATGCAGCAGCCAAGATGTTC |
| | AGTAAAGCCTTTGACAGTGGCATTATCCCCATGGAGTTTGTGAACAAGATGAAGAAGG |
| | AAGGGAAGCTGATCATGGGCATTGGTCACCGAGTGAAGTCGATAAACAACCCAGACAT |
| | GCGAGTGCAGATCCTCAAAGATTACGTCAGGCAGCACTTCCCTGCCACTCCTCTGCTC |
| | GATTATGCACTGGAAGTAGAGAAGATTACCACCTCGAAGAAGCCAAATCTTATCCTGA |
| | ATGTAGATGGTCTCATCGGAGTCCCATTTGTAGACATGCTTAGAAACTGTGGGTCCTT |
| | TACTCGGGAGGAAGCTGATGAATATATTGACATTGGAGCCCTCAATGGCATCTTTGTG |
| | CTGGGAAGGAGTATGGGGTTCATTGGACACTATCTTGATCAGAAGAGGCTGAAGCAGG |
| | CGCTGTATCGTCATCCGTGCGATGATATTTCATATGTTCTTCCGGAACACATGACCAT |
| | GTAA |
| | ORF Start: at 2 ORF Stop: TAA at 3308 |
| NOV15i, | TMSAKAISEQTGKELLYKFICTTSAIQNRFKYARVTPDTDWARLLQDHPWLLSQNLVV |
| 259856081 | |
| Protein Sequence | KPDQLIKRRGKLGLVGVNLTLDGVKSWLKPRLGQEATVGKATGFLKNFLIEPFVPHSQ |
| | AEEFYVCIYATREGDYVLFNHEGGVDVGDVDAKAQKLLVGVDEKLNPEDIKKHLLVHA |
| | PEDKKEILASFISGLFNFYEDLYFTYLEINPLVVTKDGVYVLDLAAKVDATADYICKV |
| | KWGDIEFPPPFGREAYPEEAYIADLDAKSGASLKLTLLNPKGRIWTMVAGGGASVVYS |
| | DTICDLGGVNELANYGEYSGAPSEQQTYDYAKTILSLMTREKHPDGKILIIGGSIANF |
| | TNVAATFKGIVRAIRDYQGPLKEHEVTIFVRRGGPNYQEGLRVMGEVGKTTGIPIHVF |
| | GTETHMTAIVGMALGHRPIPNQPPTAAHTANFLLNASGSTSTPAPSRTASFSESRADE |
| | VAPAKKAKPAMPQDSVPSPRSLQGKSTTLFSRHTKAIVWGMQTRAVQGMLDFDYVCSR |
| | DEPSVAAMVYPFTGDHKQKFYWGHKEILIPVFKNMADAMRKHPEVDVLINFASLRSAY |
| | DSTMETMNYAQIRTIAIIAEGIPEALTRKLIKKADQKGVTIIGPATVGGIKPGCFKIG |
| | NTGGMLDNILASKLYRPGSVAYVSRSGGMSNELNNIISRTTDGVYEGVAIGGDRYPGS |
| | TFMDHVLRYQDTPGVKMIXTVLGEIGGTEEYKICRGIKEGRLTKPIVCWCIGTCATMFS |
| | SEVQFGHAGACANQASETAVAKNQALKEAGVFVPRSFDELGEIIQSVYEDLVANGVIV |
| | PAQEVPPPTVPMDYSWARELGLIRKPASFMTSICDERGQELIYAGMPITEVFKEEMGI |
| | GGVLGLLWFQKRLPKYSCQFIEMCLMVTADHGPAVSGAHNTIICARAGKDLVSSLTSG |
| | LLTIGDRFGGALDAAAKNFSKAFDSGIIPMEFVNKMKKEGKLIMGIGHRVKSINNPDM |
| | RVQILKDYVRQHFPATPLLDYALEVEKITTSKKPNLILNVDGLIGVAFVDMLRNCGSF |
| | TREEADEYTDIGALNGIFVLGRSMGFIGHYLDQKRLKQGLYRHPWDDISYVLPEHMSM |
| | SEQ ID NO: 243 3317 bp |
| NOV15j, | CCAGAATTCCACCATGTCGGCCAAGGCAATTTCAGAGCAGACGGGCAAAGAACTCCTT |
| 256388552 | |
| DNA Sequence | TACAAGTTCATCTGTACCACCTCAGCCATCCAGAATCGGTTCAAGTATGCTCGGGTCA |
| | CTCCTGACACAGACTGGGCCCGCTTGCTGCAGGACCACCCCTGGCTGCTCAGCCAGAA |
| | CTTGGTAGTCAAGCCAGACCAGCTGATCAAACGTCGTGGAAAACTTGGTCTCGTTGGG |
| | GTCAACCTCACTCTGGATGGGGTCAAGTCCTGGCTGAAGCCACGGCTGGGACAGGAAG |
| | CCACAGTTGGCAAGGCCACAGGCTTCCTCAAGAACTTTCTGATCGAGCCCTTCGTCCC |
| | CCACAGTCAGGCTGAGGAGTTCTATGTCTGCATCTATGCCACCCGAGAAGGGGACTAC |
| | GTCCTGTTCCACCACGAGGCGGGTGTGGACGTGGGTGATGTGGACGCCAACGCCCAGA |
| | AGCTGCTTGTTGGCGTGGATGAGAAACTGAATCCTGAGGACATCAAAAAACACCTGTT |
| | GGTCCACGCCCCTGAAGACAAGAAAGAAATTCTGGCCAGTTTTATCTCCGGCCTCTTC |
| | AATTTCTACGAGGACTTGTACTTCACCTACCTCGAGATCAATCCCCTTGTAGTGACCA |
| | AAGATGGAGTCTATGTCCTTGACTTGGCGGCCAAGGTGGACGCCACTGCCGACTACAT |
| | CTGCAAAGTGAAGTGGGGTGACATCGAGTTCCCTCCCCCCTTCGGGCGGGAGGCATAT |
| | CCAGAGGAAGCCTACATTGCAGACCTCGATGCCAAAAGTGGGGCAAGCCTGAAGCTGA |
| | CCTTGCTGAACCCCAAAGGGAGGATCTGGACCATGCTGGCCGGGGGTGGCGCCTCTGT |
| | CGTGTACAGCGATACCATCTGTGATCTAGGGGGTGTCAACGAGCTGGCAAACTATGGG |
| | GAGTACTCAGGCGCCCCCAGCGAGCAGCAGACCTATGATTATGCCAAGACTATCCTCT |
| | CCCTCATGACCCGAGAGAAGCACCCAGATGGCAAGATCCTCATCATTGGAGGCAGCAT |
| | CGCAAACTTCACCAACGTGGCTGCCACGTTCAAGGGCATCGTGAGAGCAATTCGAGAT |
| | TACCAGGGCCCCCTGAAGGAGCACGAAGTCACAATCTTTGTCCGAAGAGGTGGCCCCA |
| | ACTATCAGGAGGGCTTACGGGTGATGGGAGAAGTCGGGAAGACCACTGGGATCCCCAT |
| | CCATGTCTTTGGCACAGAGACTCACATGACGGCCATTGTGGGCATGGCCCTGGGCCAC |
| | CGGCCCATCCCCAACCAGCCACCCACAGCGGCCCACACTGCAAACTTCCTCCTCAACG |
| | CCAGCGGGAGCACATCGACGCCAGCCCCCAGCAGGACAGCATCTTTTTCTGAGTCCAG |
| | GGCCGATGAGGTGGCGCCTGCAAAGAAGGCCAAGCCTGCCATGCCACAAGATTCAGTC |
| | CCAAGTCCAAGATCCCTGCAAGGAAAGAGCACCACCCTCTTCAGCCGCCACACCAAGG |
| | CCATTGTGTGGGCCATGCAGACCCGGGCCGTGCAAGGCATGCTGGACTTTGACTATGT |
| | CTGCTCCCGAGACGAGCCCTCAGTGGCTGCCATGGTCTACCCTTTCACTGGGGACCAC |
| | AAGCAGAAGTTTTACTGGGGGCACAAAGAGATCCTGATCCCTGTCTTCAAGAACATGG |
| | CTGATGCCATGAGGAAGCACCCGGAGGTAGATGTGCTCATCAACTTTGCCTCTCTCCG |
| | CTCTGCCTATGACAGCACCATGGAGACCATGAACTATGCCCAGATCCGGACCATCGCC |
| | ATCATAGCTGAAGGCATCCCTGAGGCCCTCACGAGA~AGCTGATCAAGAAGGCGGACC |
| | AGAAGGGAGTGACCATCATCGGACCTGCCACTGTTGGAGGCATCAAGCCTGGGTGCTT |
| | TAAGATTGGCAACACAGGTGGGATGCTGGACAACATCCTGGCCTCCAAACTGTACCGC |
| | CCAGGCAGCGTCGCCTATGTCTCACGTTCCGGAGGCATGTCCAACGAGCTCAACAATA |
| | TCATCTCTCGGACCACGGATGGCGTCTATGAGGGCGTGGCCATTGGTGGGGACAGGTA |
| | CCCGGGCTCCACATTCATGGATCATGTGTTACGCTATCAGGACACTCCAGGAGTCAAA |
| | ATGATTGTGGTTCTTGGAGAGATTGGGGGCACTGAGGAATATAAGATTTGCCGGGGCA |
| | TCAAGGAGGGCCGCCTCACTAAGCCCATCGTCTGCTGGTCCATCGGGACGTGTGCCAC |
| | CATGTTCTCCTCTGAGGTCCAGTTTGGCCATGCTGGAGCTTGTGCCAACCAGGCTTCT |
| | GAAACTGCAGTAGCCAAGAACCAGGCTTTGAAGGAAGCAGGAGTGTTTGTGCCCCGGA |
| | GCTTTGATGAGCTTGGAGAGATCATCCAGTCTGTATACGAAGATCTCGTGGCCAATGG |
| | AGTCATTGTACCTGCCCAGGAGGTCCCGCCCCCAACCGTGCCCATGGACTACTCCTGG |
| | GCCAGGGAGCTTGGTTTGATCCGCAAACCTGCCTCGTTCATGACCAGCATCTGCGATG |
| | AGCGAGGACAGGAGCTCATCTACGCGGGCATGCCCATCACTGAGGTCTTCAAGGAAGA |
| | GATGGGCATTGGCGGGGTCCTCGGCCTCCTCTGGTTCCAGAAAACGTTGCCTAAGTAC |
| | TCTTGCCAGTTCATTGAGATGTGTCTGATGGTGACAGCTGATCACGGGCCAGCCGTCT |
| | CTGGAGCCCACAACACCATCATTTGTGCGCGAGCTGGGAAAGACCTGGTCTCCAGCCT |
| | CACCTCGGGGCTGCTCACCATCGGGGATCGGTTTGGGGGTGCCTTGGATGCAGCAGCC |
| | AAGATGTTCAGTAAAGCCTTTGACAGTGGCATTATCCCCATGGAGTTTGTGAACAAGA |
| | TGAAGAAGGAAGGGAAGCTGATCATGGGCATTGGTCACCGAGTGAAGTCGATAAACAA |
| | CCCAGACATGCGAGTGCAGATCCTCAAAGATTACGTCAGGCAGCACTTCCCTGCCACT |
| | CCTCTGCTCGATTATGCACTGGAAGTAGAGAAGATTACCACCTCGAAGAAGCCAAATC |
| | TTATCCTGAATGTAGATGGTCTCATCGGAGTCGCATTTGTAGACATGCTTAGAAACTG |
| | TGGGTCCTTTACTCGGGAGGAAGCTGATGAATATATTCACATTGGAGCCCTCAATGGC |
| | ATCTTTGTGCTGGGAAGGAGTATGGGGTTCATTGGACACTATCTTGATCAGAAGAGGC |
| | TGAAGCAGGGGCTGTATCGTCATCCGTGGGATGATATTTCATATGTTCTTCCGGAACA |
| | CATGAGCATGT |
| | ORF Start: at 2 ORF Stop: end of sequence |
| | SEQ ID NO: 244 1106 aa MW at 121268.4kD |
| NOV15j, | QNSTMSAKAISEQTGKELLYKFICTTSAIQNRFKYARVTPDTDWARLLQDHPWLLSQN |
| 256388552 | |
| Protein Sequence | LVVKPDQLIKRRGKLGLVGVNLTLDGVKSWLKPRLGQEATVGKATGFLKNFLIEPFVP |
| | HSQAEEFYVCIYATREGDYVLFHHEGGVDVGDVDAKAQKLLVGVDEKLNPEDIKKHLL |
| | VHAPEDKKEILASFISGLFNFYEDLYFTYLEINPLVVTKDGVYVLDLAAKVDATADYI |
| | CKVKWGDIEFPPPFGREAYPEEAYIADLDAKSGASLKLTLLNPKCRIWTMVAGGGASV |
| | VYSDTICDLGGVNELANYGEYSGAPSEQQTYDYAKTILSLMTREKHPDGKILIIGGSI |
| | ANFTNVAATFKGIVRAIRDYQGPLKEHEVTIFVRRGGPMYQEGLRVMGEVGKTTGIPI |
| | HVFGTETHMTAIVGMALGHRPIPNQPPTAAHTANFLLNASGSTSTPAPSRTASFSESR |
| | ADEVAPAKKAKPAMPQDSVPSPRSLQGKSTTLFSRHTKAIVWGMQTRAVQGMLDFDYV |
| | CSRDEPSVAAMVYPFTGDHKQKFYWGHKEILIPVFKNMADAMRKHPEVDVLINFASLR |
| | SAYDSTMETMNYAQIRTIAIIAEGIPEALTRKLIKKADQKGVTIIGPATVGGIKPGCF |
| | KIGNTGGMLDNILASKLYRPGSVAYVSRSGGMSNELNNIISRTTDGVYEGVAIGCDRY |
| | PGSTFMDHVLRYQDTPGVKMIVVLGEIGGTEEYKICRGIKEGRLTKPIVCWCIGTCAT |
| | MFSSEVQFGHAGACANQASETAVAKNQALKEAGVFVPRSFDELGEIIQSVYEDLVANG |
| | VIVPAQEVPPPTVPMDYSWARELGLIRKPASFMTSICDERGQELIYAGMPTTEVFKEE |
| | MGIGGVLGLLWFQKRLPKYSCQFIEMCLMVTADHGPAVSGAHNTIICARAGKDLVSSL |
| | TSGLLTIGDRFGGALDAAAKMFSKAFDSGIIPMEFVNKMKKEGKLIMGIGHRVKSINN |
| | PDMRVQILKDYVRQHFPATPLLDYALEVEKITTSKKPNLILNVDGLIGVAFVDMLRNC |
| | CSFTREEADEYIDIGALNGIFVLGRSMGFIGHYLDQKRLKQGLYRHPWDDISYVLPEH |
| | SEQ ID NO: 245 3307 bp |
| NOV15k, | CCAGAATTCCACCATGTCCGCCAAGGCAATTTCAGAGCAGACGGGCAAAGAACTCCTT |
| 256420210 | |
| DNA Sequence | TACAAGTTCATCTGTACCACCTCAGCCATCCAGAATCGGTTCAAGTATGCTCGGGTCA |
| | CTCCTGACACAGACTGGGCCCGCTTGCTGCAGGACCACCCCTGGCTGCTCAGCCAGAA |
| | CTTGGTAGTCAAGCCAGACCAGCTGATCAAACGTCGTGGAAAACTTGGTCTCGTTGGG |
| | GTCAACCTCACTCTGGATGGGGTCAAGTCCTGGCTGAAGCCACGGCTGGGACAGCAAG |
| | CCACAGTTGGCAAGGCCACAGGCTTCCTCAAGAACTTTCTGATCGAGCCCTTCGTCCC |
| | CCACAGTCAGGCTGAGGAGTTCTATGTCTGCATCTATGCCACCCGAGAAGGGGACTAC |
| | GTCCTGTTCCACCACGAGGGGGGTGTGGACGTGGGTGATGTGGACGCCAAGGCCCAGA |
| | AGCTGCTTGTTGGCGTGGATGAGAAACTGAATCCTGAGGACATCAAAAAACACCTGTT |
| | GGTCCACGCCCCTGAAGACAAGAAAGAAATTCTGGCCAGTTTTATCTCCGCCCTCTTC |
| | AATTTCTACGAGGACTTGTACTTCACCTACCTCGAGATCAATCCCCTTGTAGTCACCA |
| | AAGATGGAGTCTATGTCCTTGACTTGGCGGCCAAGGTGGACGCCACTGCCGACTACAT |
| | CTGCAAAGTGAAGTGGGGTGACATCGAGTTCCCTCCCCCCTTCGGGCGGGAGGCATAT |
| | CCAGAGGAAGCCTACATTGCAGACCTCGATGCCAAAAGTGGGGCAAGCCTGAAGCTGA |
| | CCTTGCTGAACCCCAAAGGGAGGATCTGGACCATGGTGGCCGGGGGTGGCGCCTCTGT |
| | CGTGTACAGCGATACCATCTGTGATCTAGGGGGTGTCAACGAGCTGGCAAACTATGGG |
| | GAGTACTCAGGCGCCCCCAGCGAGCAGCAGACCTATGACTATGCCAAGACTATCCTCT |
| | CCCTCATGACCCGAGAGAAGCACCCAGATGGCAAGATCCTCATCATTGGAGGCAGCAT |
| | CGCAAACTTCACCAACGTGGCTGCCACGTTCAAGGGCATCGTGAGAGCAATTCGAGAT |
| | TACCAGGGCCCCCTGAAGGAGCACGAAGTCACAATCTTTGTCCGAAGAGGTGGCCCCA |
| | ACTATCAGGAGGGCTTACGGGTGATGGGAGAAGTCGGGAAGACCACTGGGATCCCCAT |
| | CCATGTCTTTGGCACAGAGACTCACATGACGGCCATTGTGGGCATGGCCCTGGGCCAC |
| | CGGCCCATCCCCAACCAGCCACCCACAGCGGCCCACACTGCAAACTTCCTCCTCAACG |
| | CCAGCGGGAGCACATCGACGCCAGCCCCCAGCAGGACAGCATCTTTTTCTGAGTCCAG |
| | GGCCGATGAGGTGGCGCCTGCAAAGAAGGCCAAGCCTGCCATGCCACAAGGAAAGAGC |
| | ACCACCCTCTTCAGCCGCCACACCAACGCCATTGTGTGGGGCATGCAGACCCGGGCCG |
| | TGCAAGGCATGCTGGACTTTCACTATGTCTGCTCCCGAGACGAGCCCTCAGTGGCTGC |
| | CATGGTCTACCCTTTCACTGGGGACCACAAGCAGAAGTTTTACTGGGGGCACAAAGAG |
| | ATCCTGATCCCTGTCTTCAAGAACATGGCTGATGCCATGAGGAAGCACCCGGAGGTAG |
| | ATGTGCTCATCAACTTTGCCTCTCTCCGCTCTGCCTATGACAGCACCATGGAGACCAT |
| | GAACTATGCCCAGATCCGGACCATCGCCATCATAGCTGAAGGCATCCCTGAGGCCCTC |
| | ACGAGAAAGCTGATCAAGAAGGCGGACCAGAAGGGAGTGACCATCATCGGACCTGCCA |
| | CTGTTGGAGGCATCAAGCCTGGGTGCTTTAAGATTGGCAACACAGGTGGGATGCTGGA |
| | CAACATCCTGGCCTCCAAACTGTACCGCCCAGGCAGCGTGGCCTATGTCTCACGTTCC |
| | GGAGGCATGTCCAACGAGCTCAACAATATCATCTCTCGGACCACGGATGGCGTCTATG |
| | AGGGCGTGGCCATTGGTGGGGACAGGTACCCGGGCTCCACATTCATGGATCATGTGTT |
| | ACGCTATCAGGACACTCCAGGAGTCAAAATGATTGTGGTTCTTGGAGAGATTGGGGGC |
| | ACTGAGGAATATAAGATTTGCCGGGGCATCAAGGAGGGCCGCCTCACTAAGCCCATCG |
| | TCTGCTGGTGCATCGGGACGTGTGCCACCATGTTCTCCTCTGAGGTCCAGTTTGGCCA |
| | TGCTGGAGCTTGTGCCAACCAGGCTTCTGAAACTGCAGTAGCCAAGAACCAGGCTTTG |
| | AAGGAAGCAGGAGTGTTTGTGCCCCGGAGCTTTGATGAGCTTGGAGAGATCATCCAGT |
| | CTGTATACGAAGATCTCGTGGCCAATGGAGTCATTGTACCTGCCCAGGAGGTGCCGCC |
| | CCCAACCGTGCCCATGGACTACTCCTGGGCCAGGGAGCTTGGTTTGATCCGCAAACCT |
| | GCCTCGTTCATGACCAGCATCTGCGATGAGCGAGGACAGGAGCTCATCTACGCGGGCA |
| | TGCCCATCACTGAGGTCTTCAAGGAAGAGATGGGCATTGGCGGGGTCCTCGGCCTCCT |
| | CTGGTTCCAGAAAAGGTTGCCTAAGTACTCTTGCCAGTTCATTGAGATGTGTCTGATG |
| | GTGACAGCTGATCACGGGCCAGCCGTCTCTGGAGCCCACAACACCATCATTTGTGCGC |
| | GAGCTGGGAAAGACCTGGTCTCCAGCCTCACCTCGGGGCTGCTCACCATCGGGGATCG |
| | GTTTGGCGGTGCCTTGGATGCAGCAGCCAACATGTTCAGTAAAGCCTTTGACAGTGGC |
| | ATTATCCCCATGGAGTTTGTGAACAAGATGAAGAAGGAAGGGAAGCTGATCATGGGCA |
| | TTGGTCACCCAGTGAAGTCGATAAACAACCCAGACATGCGAGTGCAGATCCTCAAAGA |
| | TTACGTCAGGCAGCACTTCCCTGCCACTCCTCTGCTCGATTATGCACTGGAAGTAGAG |
| | AAGATTACCACCTCGAAGAAGCCAAATCTTATCCTGAATGTAGATGGTCTCATCGGAG |
| | TCGCATTTGTAGACATGCTTAGAAACTGTGGGTCCTTTACTCGGGAGGAAGCTGATGA |
| | ATATATTGACATTGGAGCCCTCAATGGCATCTTTGTGCTGGGAAGGAGTATGGGGTTC |
| | ATTGGACACTATCTTGATCAGAAGAGGCTGAAGCAGGGGCTGTATCGTCATCCGTGGG |
| | ATGATATTTCATATGTTCTTCCGGAACACATGAGCATGTAAGCGGCCGCTTTTTTCCT |
| | T |
| | ORF Start: at 2 ORF Stop: TAA at 3287 |
| | SEQ ID NO: 246 1095 aa MW at 120201.2kD |
| NOV15k, | QNSTMSAKAISEQTGKELLYKFICTTSAIQNRFKYARVTPDTDWARLLQDHPWLLSQN |
| 256420210 | |
| Protein Sequence | LVVKPDQLIKRRGKLGLVGVNLTLDGVKSWLKPRLGQEATVGKATGFLKNFLIEPFVP |
| | HSQAEEFYVCIYATREGDYVLFHHEGGVDVGDVDAKAQKLLVGVDEKLNPEDIKKHLL |
| | VHAPEDKKEILASFISGLFNFYEDLYFTYLEINPLVVTKDGVYVLDLAAKVDATADYI |
| | CKVKWGDIEFPPPFGREAYPEEAYIADLDAKSGASLKLTLLNPKGRIWTMVAGGGASV |
| | VYSDTICDLGGVNELANYGEYSGAPSFQQTYDYAKTILSLMTREKHPDGKILIIGGSI |
| | ANFTNXTAATFKGIVRAIRDYQGPLKEHEVTIFVRRGGPNYQEGLRVMGEVGKTTGIPT |
| | HVFGTETHMTAIVGMALGHRPIPNQPPTAAHTANFLLNASGSTSTPAPSRTASFSESR |
| | ADEVAPAKKAKPAMPQGKSTTLFSRHTKAIVWGMQTRAVQGMLDFDYVCSRDEPSVAA |
| | MVYPFTGDHKQKFYWGHKEILIPVFKNMADAMRKHPEVDVLINFASLRSAYDSTMETM |
| | NYAQIRTIATIAEGIPEALTRKLIKKADQKGVTIIGPATVGGIKPGCFKIGNTGGMLD |
| | NILASKLYRPCSVAYVSRSGGMSNELNNIISRTTDGVYEGVAIGGDRYPGSTFMDHVL |
| | RYQDTPGVKMIVVLGEIGGTEEYKICRGIKEGRLTKPIVCWCIGTCATMFSSEVQFGH |
| | AGACANQASETAVAKNQALKEAGVFVPRSFDELGEIIQSVYEDLVANGVIVPAQEVPP |
| | PTVPMDYSWARELGLIRKPASFMTSICDERGQELIYAGMPITEVFKEEMGIGGVLGLL |
| | WFQKRLPKYSCQFIEMCLMVTADHGPAVSGAHNTIICARAGKDLVSSLTSGLLTIGDR |
| | FGGALDAAAKMFSKAFDSGIIPMEFVNKIVIKKEGKLIMGIGHRVKSINNPDMRVQILKD |
| | YVRQHFPATPLLDYALEVEKITTSKKPNLILNVDGLIGVAFVDMLRNCGSFTREEADE |
| | YIDIGALNGIFVLGRSMGFIGHYLDQKRLKQGLYRHPWDDISYVLPEHMSM |
| | SEQ ID NO: 247 2290 bp |
| NOV15l, | CCAGAATTCCACCATGTCGGCCAAGGCAATTTCAGAGCAGACGGGCAAAGAACTCCTT |
| 256202925 | |
| DNA Sequence | TACAAGTTCATCTGTACCACCTCAGCCATCCAGAATCGGTTCAAGTATGCTCGCGTCA |
| | CTCCTGACACAGACTGGGCCCGCTTGCTGCAGGACCACCCCTGGCTGCTCAGCCAGAA |
| | CTTGGTAGTCAAGCCAGACCAGCTGATCAAACGTCGTGGAAAACTTGGTCTCGTTGGG |
| | GTCAACCTCACTCTGGATGGGGTCAAGTCCTGGCTGAAGCCACGGCTGGGACAGGAAG |
| | CCACAGTGAGTGGGCATGGGGTCAAGATGAACGTGTGTGGTAACAGAAGCAAATATGG |
| | TCACCTTCAGGTTGGCAAGGCCACAGGCTTCCTCAAGAACTTTCTGATCGAGCCCTTC |
| | GTCCCCCACAGTCAGGCTGAGGAGTTCTATGTCTGCATCTATGCCACCCGAGAAGGGG |
| | ACTACGTCCTGTTCCACCACGAGGGGGGTGTGGACGTGGGTGATGTGGACGCCAAGGC |
| | CCAGAAGCTGCTTGTTGGCGTGGATCAGAAACTGAATCCTGAGGACATCAAAAAACAC |
| | CTGTTGCTCCACGCCCCTGAAGACAAGAAAGAAATTCTGGCCAGTTTTATCTCCGGCC |
| | TCTTCAATTTCTACGAGGACTTGTACTTCACCTACCTCGAGATCAATCCCCTTGTAGT |
| | GACCAAAGATGGAGTCTATGTCCTTGACTTGGCGGCCAAGGTGGACGCCACTGCCGAC |
| | TACATCTGCAAAGTGAAGTGGGGTGACATCGAGTTCCCTCCCCCCTTCGGGCGGGAGG |
| | CATATCCAGAGGAAGCCTACATTGCAGACCTCGACGCCAAAAGTGGGGCAAGCCTGAA |
| | GCTGACCTTGCTGAACCCCAAAGGGAGGATCTGGACCATGGTGGCCGGGGGTGGCGCC |
| | TCTGTCGTGTACAGCGATACCATCTGTGATCTAGGGGGTGTCAACGAGCTGGCAAACT |
| | ATGGGGAGTACTCAGGCGCCCCCAGCGAGCAGCAGACCTATGACTATGCCAAGACTAT |
| | CCTCTCCCTCATGACCCGAGAGAAGCACCCAGATGGCAAGATCCTCATCATTGGAGGC |
| | AGCATCGCAAACTTCACCAACGTGGCTGCCACGTTCAAGGGCATCGTGAGAGCAATTC |
| | GAGATTACCAGGGCCCCCTGAAGGAGCACGAAGTCACAATCTTTGTCCGAAGAGGTGG |
| | CCCCAACTATCAGGAGGGCTTACGGGTGATGGGAGAAGTCGGGAAGACCACTGGGATC |
| | CCCATCCATGTCTTTGGCACAGAGACTCACATGACGGCCATTGTGGGCATGGCCCTGG |
| | GCCACCGGCCCATCCCCAACCAGCCACCCACAGCGGCCCACACTGCAAACTTCCTCCT |
| | CAACGCCAGCGGGAGCACATCGACGCCAGCCCCCAGCAGGACAGCATCTTTTTCTGAG |
| | TCCAGGGCCGATGAGGTGGCGCCTGCAAAGAAGGCCAAGCCTGCCATGCCACAAGGAA |
| | AGAGCACCACCCTCTTCAGCCGCCACACCAAGGCCATTGTGTGGGGCATGCAGACCCG |
| | GGCCGTGCAAGGCATGCTGGACTTTGACTATGTCTGCTCCCGAGACGAGCCCTCAGTG |
| | GCTGCCATGGTCTACCCTTTCACTGGGGACCACAAGCAGAAGTTTTACTGGGGGCACA |
| | AAGAGATCCTGATCCCTGTCTTCAAGAACATGGCTGATGCCATGAGGAAGCACCCGGA |
| | GGTAGATGTGCTCATCAACTTTGCTTCTCTCCGCTCTGCCTTGGATGCAGCAGCCAAG |
| | ATGTTCAGTAAAGCCTTTGACAGTGGCATTATCCCCATGGAGTTTGTGAACAAGATGA |
| | AGAAGGAAGGGAAGCTGATCATGGGCATTGGTCACCGAGTGAAGTCGATAAACAACCC |
| | AGACATGCGAGTGCGGATCCTCAAAGATTACGTCAGGCAGCACTTCCCTGCCACTCCT |
| | CTGCTCGATTATGCACTGGAAGTAGAGAAGATTACCACCTCGAAGAAGCCAAATCTTA |
| | TCCTGAATGTAGATGGTCTCATCGGAGTCGCATTTGTAGACATGCTTAGAAACTGTCG |
| | GTCCTTTACTCGGGAGGAAGCTGATGAATATATTGACATTGGAGCCCTCAATGGCATC |
| | TTTGTGCTGGGAAGGAGTATGGGGTTCATTGGACACTATCTTGATCAGAAGAGGCTGA |
| | AGCAGGGGCTGTATCGTCATCCGTGGGATGATATTTCATATGTTCTTCCGGAACACAT |
| | GAGCATGTAAGCGGCCGCTTTTTTCCTT |
| | ORF Start:at 2 ORF Stop: TAA at 2270 |
| | SEQ ID NO: 248 756 aa MW at 83890.7kD |
| NOV15l, | QNSTMSAKAISEQTGKELLYKFICTTSAIQNRFKYARVTPDTDWARLLQDHPWLLSQN |
| 256202925 | |
| Protein Sequence | LVVKPDQLIKRRGKLGLVGVNLTLDGVKSWLKPRLGQEATVSGHGVKMNVCGNRSKYG |
| | HLQVGKATGFLKNFLIEPFVPHSQAEEFYVCIYATREGDYVLFHHEGGVDVGDVDAKA |
| | QKLLVGVDEKLNPEDIKKHLLVHAPEDKKEILASFISGLFNFYEDLYFTYLEINPLVV |
| | TKDGVYVLDLAAKVDATADYICKVKWGDIEFPPPFGREAYPEEAYIADLDAKSGASLK |
| | LTLLNPKGRIWTMVAGGGASVVYSDTICDLGGVNELANYGEYSGAPSEQQTYDYAKTI |
| | LSLMTREKHPDGKILIIGGSIANFTNVAATFKGIVRAIRDYQGPLKEHEVTIFVRRGG |
| | PNYQEGLRVMGEVGKTTGIPIHVFGTETHMTAIVGMALGHRPIPNQPPTAAHTANFLL |
| | NASGSTSTPAPSRTASFSESRADEVAPAKKAKPAMPQGKSTTLFSRHTKAIVWGMQTR |
| | AVQGMLDFDYVCSRDEPSVAAIVIVYPFTGDHKQKFYWGHKEILIPVFKNMADAMRKHPE |
| | VDVLINFASLRSALDAAAKNFSKAFDSGIIPMEFVMKMKKEGKLIMGIGHRVKSINNP |
| | DMRVRILKDYVRQHFPATPLLDYALEVEKITTSKKPNLILNVDGLIGVAFVDMLRNCG |
| | SFTREEADEYIDIGALNGIFVLGRSMGFIGHYLDQKRLKQGLYRHPWDDISYVLPEHM |
| | SM |
| | SEQ ID NO: 249 3368 bp |
| NOV15m, | CCCGGTCCGAAGCGCGCGGATTCCACCATGTCGGCCAAGGCAATTTCAGAGCAGACGG |
| 296463359 | |
| DNA Sequence | GCAAAGAACTCCTTTACAAGTTCATCTGTACCACCTCAGCCATCCAGAATCGGTTCAA |
| | GTATGCTCGGGTCACTCCTGACACAGACTGGGCCCGCTTGCTGCAGGACCACCCCTGG |
| | CTGCTCAGCCAGAACTTGGTAGTCAAGCCAGACCAGCTGATCAAACGTCGTGGAAAAC |
| | TTGGTCTCGTTGGGGTCAACCTCACTCTGGATGGGGTCAAGTCCTGGCTGAAGCCACG |
| | GCTGGGACAGGAAGCCACAGTTGGCAAGGCCACAGGCTTCCTCAAGAACTTTCTGATC |
| | GAGCCCTTCGTCCCCCACAGTCAGGCTGAGGAGTTCTATGTCTGCATCTATCCCACCC |
| | GAGAAGGGGACTACGTCCTGTTCCACCACGAGGGGGGTGTGGACGTGGGTGATGTGGA |
| | CGCCAAGGCCCAGAAGCTGCTTGTTGGCGTGGATGAGAAACTGAATCCTGAGGACATC |
| | AAAAAACACCTGTTGGTCCACGCCCCTGAAGACAAGAAAGAAATTCTGGCCAGTTTTA |
| | TCTCCGGCCTCTTCAATTTCTACGAGGACTTGTACTTCACCTACCTCGAGATCAATCC |
| | CCTTGTAGTGACCAAAGATGGAGTCTATGTCCTTGACTTGGCGGCCAAGGTGGACGCC |
| | ACTGCCGACTACATCTGCAAAGTGAAGTGGGCTGACATCGAGTTCCCTCCCCCCTTCG |
| | GGCGGGAGGCATATCCAGAGGAAGCCTACATTGCAGACCTCGATGCCAAAAGTGGGGC |
| | AAGCCTGAAGCTGACCTTGCTGAACCCCAAAGGGAGGATCTGGACCATGGTGGCCGGG |
| | GGTGGCGCCTCTGTCGTGTACAGCGATACCATCTGTGATCTAGGGGGTGTCAACGAGC |
| | TGGCAAACTATGGGGAGTACTCAGGCGCCCCCAGCGAGCAGCAGACCTATGATTATGC |
| | CAAGACTATCCTCTCCCTCATGACCCGAGAGAAGCACCCAGATGGCAAGATCCTCATC |
| | ATTGGACGCAGCATCGCAAACTTCACCAACGTGGCTGCCACGTTCAAGGGCATCGTGA |
| | GACCAATTCGAGATTACCAGGGCCCCCTGAAGGAGCACGAAGTCACAATCTTTGTCCG |
| | AAGAGGTGGCCCCAACTATCAGGAGGGCTTACGGGTGATGGGAGAAGTCGGGAACACC |
| | ACTGGGATCCCCATCCATGTCTTTGGCACAGAGACTCACATGACGGCCATTGTGGGCA |
| | TGGCCCTGGGCCACCGGCCCATCCCCAACCAGCCACCCACAGCGGCCCACACTGCAAA |
| | CTTCCTCCTCAACGCCAGCGGGAGCACATCGACGCCAGCCCCCAGCAGGACAGCATCT |
| | TTTTCTGAGTCCAGGGCCGATGAGGTGGCGCCTGCAAAGAAGGCCAAGCCTGCCATGC |
| | CACAAGATTCAGTCCCAAGTCCAAGATCCCTGCAAGGAAAGAGCACCACCCTCTTCAG |
| | CCGCCACACCAAGGCCATTGTGTGGGGCATGCAGACCCGGGCCGTGCAAGGCATGCTG |
| | GACTTTGACTATGTCTGCTCCCGAGACGAGCCCTCAGTGGCTGCCATGGTCTACCCTT |
| | TCACTGGGGACCACAAGCAGAAGTTTTACTGGGGGCACAAAGAGATCCTGATCCCTGT |
| | CTTCAAGAACATGGCTGATGCCATGAGGAAGCACCCGGAGGTAGATGTGCTCATCAAC |
| | TTTGCCTCTCTCCGCTCTGCCTATGACAGCACCATGGAGACCATCAACTATGCCCAGA |
| | TCCGGACCATCGCCATCATAGCTGAAGCCATCCCTGAGGCCCTCACGAGAAAGCTGAT |
| | CAAGAAGGCGGACCAGAAGGGAGTGACCATCATCGGACCTGCCACTGTTGGAGGCATC |
| | AAGCCTGGGTGCTTTAAGATTGGCAACACAGGTGGGATGCTGGACAACATCCTGGCCT |
| | CCAAACTGTACCGCCCAGGCAGCGTGGCCTATGTCTCACGTTCCGGAGGCATGTCCAA |
| | CGAGCTCAACAATATCATCTCTCGGACCACGGATGGCGTCTATGAGGGCGTGGCCATT |
| | CGTGGGGACAGGTACCCGGGCTCCACATTCATGGATCATGTGTTACGCTATCAGGACA |
| | CTCCAGGAGTCAAAATGATTGTGGTTCTTGGAGAGATTGGGGGCACTGAGGAATATAA |
| | GATTTGCCCGGGCATCAAGGAGGGCCGCCTCACTAAGCCCATCGTCTGCTGGTGCATC |
| | GGGACGTGTGCCACCATGTTCTCCTCTGAGGTCCAGTTTGGCCATGCTGGAGCTTGTG |
| | CCAACCAGGCTTCTGAAACTGCAGTAGCCAAGAACCAGGCTTTGAAGGAAGCAGGAGT |
| | GTTTGTGCCCCGGAGCTTTGATGAGCTTGGAGAGATCATCCAGTCTGTATACGAAGAT |
| | CTCGTGGCCAATGGAGTCATTGTACCTGCCCAGGAGGTGCCGCCCCCAACCGTGCCCA |
| | TGGACTACTCCTGGGCCAGGGAGCTTGGTTTGATCCGCAAACCTGCCTCGTTCATGAC |
| | CAGCATCTGCGATGAGCGAGGACAGGAGCTCATCTACGCGGGCATGCCCATCACTGAG |
| | GTCTTCAAGGAAGAGATGGGCATTGGCGGGGTCCTCGGCCTCCTCTGGTTCCAGAAAA |
| | GGTTGCCTAAGTACTCTTGCCAGTTCATTGAGATGTGTCTGATGGTGACAGCTGATCA |
| | CGGGCCAGCCGTCTCTGGAGCCCACAACACCATCATTTGTGCGCGAGCTGGGAAAGAC |
| | CTGGTCTCCAGCCTCACCTCGGGGCTGCTCACCATCGGGGATCGGTTTGGGGGTGCCT |
| | TGGATGCAGCAGCCAAGATGTTCAGTAAAGCCTTTGACAGTGGCATTATCCCCATGGA |
| | GTTTGTGAACAAGATGAAGAAGGAAGGGAAGCTGATCATGGGCATTGGTCACCGAGTG |
| | AAGTCGATAAACAACCCAGACATGCGAGTGCAGATCCTCAAAGATTACGTCAGGCAGC |
| | ACTTCCCTGCCACTCCTCTGCTCGATTATGCACTGGAAGTAGAGAAGATTACCACCTC |
| | GAAGAAGCCAAATCTTATCCTGAATGTAGATGGTCTCATCGGAGTCGCATTTGTAGAC |
| | ATGCTTAGAAACTGTGGGTCCTTTACTCGGGAGGAAGCTGATGAATATATTGACATTG |
| | GAGCCCTCAATGGCATCTTTGTGCTGGGAAGGAGTATGGGGTTCATTGGACACTATCT |
| | TGATCAGAAGAGGCTGAAGCAGGGGCTGTATCGTCATCCGTGGGATGATATTTCATAT |
| | GTTCTTCCGGAACACATGAGCATGCATCATCACCACCATCACTAAGCGCCCGCTTTCG |
| | AATC |
| | ORF Start: at 1 ORF Stop: TAA at 3349 |
| | SEQ ID NO: 250 1116 aa MW at 122570.8kD |
| NOV15m, | PGPKRADSTMSAKAISEQTGKELLYKFICTTSAIQNRFKYARVTPDTDWARLLQDHPW |
| 296463359 | |
| Protein Sequence | LLSQNLVVKPDQLIKRRGKLGLVGVNLTLDGVKSWLKPRLGQEATVGKATGFLKNFLI |
| | EPFVPHSQAEEFYVCIYATREGDYVLFHHEGGVDVGDVDAKAQKLLVGVDEKLNPEDI |
| | KKHLLVHAPEDKKEILASFISGLFNFYEDLYFTYLEINPLVVTKDGVYVLDLAAKVDA |
| | TADYICKVKWGDIEFPPPFGREAYPEEAYIADLDAKSGASLKLTLLNPKGRIWTMVAG |
| | GGASVVYSDTICDLGGVNELANYGEYSGAPSEQQTYDYAKTILSLMTREKHPDGKILI |
| | IGGSIANFTNVAATFKGIVRAIRDYQGPLKEHEVTIFVRRGGPNYQEGLRVMGEVGKT |
| | TGIPIHVFGTETHMTAIVGMALGHRPIPNQPPTAAHTANFLLNASGSTSTPAPSRTAS |
| | FSESRADEVAPAKKAKPAMPQDSVPSPRSLQGKSTTLFSRHTKAIVWGMQTRAVQGML |
| | DFDYVCSRDEPSVAAMVYPFTGDHKQKFYWGHKEILIPVFKNMADAMRKHPEVDVLIN |
| | FASLRSAYDSTMETMNYAQIRTIAIIAEGIPEALTRKLIKKADQKGVTIIGPATVGGI |
| | KPGCFKIGNTGGMLDNILASKLYRPCSVAYVSRSGGMSNELNNIISRTTDGVYEGVAI |
| | GGDRYPGSTFMDHVLRYQDTPGVKMIVVLGEIGGTEEYKICRGIKEGRLTKPIVCWCT |
| | GTCATMFSSEVQFGHAGACANQASETAVAKNQALKEAGVFVPRSFDELGEIIQSVYED |
| | LVANGVIVPAQEVPPPTVPMDYSWARELGLIRKPASFMTSICDERGQELIYAGMPITE |
| | VFKEEMGIGGVLGLLWFQKRLPKYSCQFIEMCLMVTADHGPAVSGAHNTIICARAGKD |
| | LVSSLTSGLLTIGDRFGGALDAAAKMFSKAFDSGIIPMEFVNKMKKEGKLIMGIGHRV |
| | KSINNPDMRVQILKDYVRQHFPATPLLDYALEVEKITTSKKPNLILNVDGLIGVAFVD |
| | MLRNCGSFTREEADEYIDIGALNGIFVLGRSMGFIGHYLDQKRLKQGLYRHPWDDISY |
| | VLPEHMSMHHHHHH |
| | SEQ ID NO: 251 3313 bp |
| NOV15n, | TTCCACCATGTCCGCCAAGGCAATTTCAGAGCAGACGGGCAAAGAACTCCTTTACAAG |
| 263470992 | |
| DNA Sequence | TTCATCTGTACCACCTCAGCCATCCAGAATCGGTTCAAGTATGCTCGGGTCACTCCTG |
| | ACACAGACTGGGCCCGCTTGCTGCAGGACCACCCCTGGCTGCTCAGCCAGAACTTGGT |
| | AGTCAAGCCAGACCAGCTGATCAAACGTCGTGGAAAACTTGGTCTCGTTGGGGTCAAC |
| | CTCACTCTGGATGGGGTCAAGTCCTGGCTGAAGCCACGGCTGGGACAGGAAGCCACAG |
| | TTGGCAAGGCCACAGGCTTCCTCAAGAACTTTCTGATCGAGCCCTTCGTCCCCCACAG |
| | TCAGGCTGAGGAGTTCTATGTCTGCATCTATGCCACCCGAGAAGGGGACTACGTCCTG |
| | TTCCACCACGAGGGGGGTGTGGACGTGGGTGATGTGGACGCCAAGGCCCAGAAGCTGC |
| | TTGTTGGCGTGGATGAGAAACTGAATCCTGAGGACATCAAAAAACACCTGTTGGTCCA |
| | CCCCCCTGAAGACAAGAAAGAAATTCTGGCCAGTTTTATCTCCGGCCTCTTCAATTTC |
| | TACGAGGACTTGTACTTCACCTACCTCGAGATCAATCCCCTTGTAGTGACCAAAGATG |
| | GAGTCTATGTCCTTGACTTGGCGGCCAAGGTGGACGCCACTGCCGACTACATCTGCAA |
| | AGTGAAGTGGGGTGACATCGAGTTCCCTCCCCCCTTCGGGCGGGAGGCATATCCAGAG |
| | GAAGCCTACATTGCAGACCTCGATGCCAAAAGTGGGGCAAGCCTGAAGCTGACCTTGC |
| | TGAACCCCAAAGGGAGGATCTGGACCATGGTGGCCGGGGGTGGCGCCTCTGTCGTGTA |
| | CAGCGATACCATCTGTGATCTAGGGGGTGTCAACGAGCTGGCAAACTATGGGGAGTAC |
| | TCAGGCGCCCCCAGCGAGCAGCAGACCTATGATTATGCCAAGACTATCCTCTCCCTCA |
| | TGACCCGAGAGAAGCACCCAGATGGCAAGATCCTCATCATTGGAGGCAGCATCGCAAA |
| | CTTCACCAACGTGGCTGCCACGTTCAAGGGCATCGTGAGAGCAATTCGAGATTACCAG |
| | GGCCCCCTGAAGGAGCACGAAGTCACAATCTTTGTCCGAAGAGGTGGCCCCAACTATC |
| | AGGAGGGCTTACGGGTGATGGGAGAAGTCGGGAAGACCACTGGGATCCCCATCCATGT |
| | CTTTGGCACAGAGACTCACATGACGGCCATTGTGGGCATGGCCCTGGGCCACCGGCCC |
| | ATCCCCAACCAGCCACCCACAGCGGCCCACACTGCAAACTTCCTCCTCAACGCCAGCG |
| | GGAGCACATCGACGCCAGCCCCCAGCAGGACAGCATCTTTTTCTGAGTCCAGGGCCGA |
| | TGAGGTGGCGCCTGCAAAGAAGGCCAAGCCTGCCATGCCACAAGATTCAGTCCCAAGT |
| | CCAAGATCCCTGCAAGGAAAGAGCACCACCCTCTTCAGCCGCCACACCAAGGCCATTG |
| | TGTGGGGCATGCAGACCCGGGCCGTGCAAGGCATGCTGGACTTTGACTATGTCTGCTC |
| | CCGAGACGAGCCCTCAGTGGCTGCCATGGTCTACCCTTTCACTGGGGACCACAAGCAG |
| | AAGTTTTACTGGGGGCACAAAGAGATCCTGATCCCTGTCTTCAAGAACATGGCTGATG |
| | CCATGAGGAAGCACCCGGAGGTAGATGTGCTCATCAACTTTGCCTCTCTCCGCTCTGC |
| | CTATGACAGCACCATCGAGACCATGAACTATGCCCAGATCCGGACCATCGCCATCATA |
| | GCTGAAGGCATCCCTGAGGCCCTCACGAGAAAGCTGATCAAGAAGGCGGACCAGAAGG |
| | GAGTGACCATCATCGGACCTGCCACTGTTGGAGGCATCAAGCCTGGGTGCTTTAAGAT |
| | TGGCAACACAGGTGGGATGCTGGACAACATCCTGGCCTCCAAACTGTACCGCCCAGGC |
| | AGCGTGGCCTATGTCTCACGTTCCGGAGGCATGTCCAACGAGCTCAACAATATCATCT |
| | CTCGGACCACGGATGGCGTCTATGAGGGCGTGGCCATTGGTGGGGACAGGTACCCGGG |
| | CTCCACATTCATGGATCATGTGTTACGCTATCAGGACACTCCAGGAGTCAAAATGATT |
| | GTGGTTCTTGGAGAGATTGGGGGCACTGAGGAATATAAGATTTGCCGGGGCATCAAGG |
| | AGGGCCGCCTCACTAAGCCCATCGTCTGCTGGTGCATCGGGACGTGTGCCACCATGTT |
| | CTCCTCTGAGGTCCAGTTTGGCCATGCTGGAGCTTGTGCCAACCAGGCTTCTGAAACT |
| | GCAGTAGCCAAGAACCAGGCTTTGAAGGAAGCAGGAGTGTTTGTGCCCCGGAGCTTTG |
| | ATGAGCTTGGAGAGATCATCCAGTCTGTATACGAAGATCTCGTGGCCAATGGAGTCAT |
| | TGTACCTGCCCAGGAGGTGCCGCCCCCAACCGTGCCCATGGACTACTCCTGGGCCAGG |
| | GAGCTTGGTTTGATCCGCAAACCTGCCTCGTTCATGACCAGCATCTGCGATGAGCGAG |
| | GACAGGAGCTCATCTACGCGGGCATGCCCATCACTGAGGTCTTCAAGGAAGAGATGGG |
| | CATTGGCGGGGTCCTCGGCCTCCTCTGGTTCCAGAAAAGGTTGCCTAAGTACTCTTGC |
| | CAGTTCATTGAGATGTGTCTGATGGTGACAGCTGATCACGGGCCAGCCGTCTCTGGAG |
| | CCCACAACACCATCATTTGTGCGCGAGCTGGGAAAGACCTGGTCTCCAGCCTCACCTC |
| | GGGGCTGCTCACCATCGGGGATCGGTTTGGGGGTGCCTTGGATGCAGCAGCCAAGATG |
| | TTCAGTAAAGCCTTTGACAGTGGCATTATCCCCATGGAGTTTGTGAACAAGATGAAGA |
| | AGGAAGGGAAGCTGATCATGGGCATTGGTCACCGAGTGAAGTCGATAAACAACCCAGA |
| | CATGCGAGTGCAGATCCTCAAAGATTACGTCAGGCAGCACTTCCCTGCCACTCCTCTG |
| | CTCGATTATGCACTGGAAGTAGAGAAGATTACCACCTCGAAGAAGCCAAATCTTATCC |
| | TGAATGTAGATGGTCTCATCGGAGTCGCATTTGTAGACATGCTTAGAAACTGTGGGTC |
| | CTTTACTCGGGAGGAAGCTGATGAATATATTGACATTGGAGCCCTCAATGGCATCTTT |
| | GTGCTGGGAAGGAGTATGGGGTTCATTGGACACTATCTTGATCAGAAGAGGCTGAAGC |
| | AGGGGCTGTATCGTCATCCGTGGGATGATATTTCATATGTTCTTCCGGAACACATGAG |
| | CATGTAA |
| | ORF Start: at 2 ORF Stop: TAA at 3311 |
| | SEQ ID NO: 252 1103 aa MW at 121026.1kD |
| NOV15n, | STMSAKAISEQTGKELLYKFICTTSAIQNRFKYARVTPDTDWARLLQDHPWLLSQNLV |
| 263470992 | |
| Protein Sequence | VKPDQLIKRRGKLGLVGVNLTLDGVKSWLKPRLGQEATVGKATGFLKNFLIEPFVPHS |
| | QAEEFYVCIYATREGDYVLFHHEGGVDVGDVDAKAQKLLVGVDEKLNPEDIKKHLLVH |
| | APEDKKEILASFISGLFNFYEDLYFTYLEINPLVVTKDGVYVLDLAAKVDATADYICK |
| | VKWGDIEFPPPFGREAYPEEAYIADLDAKSGASLKLTLLNPKGRIWTMVAGGGASVVY |
| | SDTICDLGGVNELANYGEYSGAPSEQQTYDYAKTILSLMTREKHPDGKILIIGGSIAN |
| | FTNVAATFKGIVRAIRDYQGPLKEHEVTIFVRRGGPNYQEGLRVMGEVGKTTGIPIIW |
| | FGTETHMTAIVGMALGHRPIPNQPPTAAHTANFLLNASGSTSTPAPSRTASFSESRAD |
| | EVAPAKKAKPAMPQDSVPSPRSLQGKSTTLFSRHTKAIVWGMQTRAVQGMLDFDYVCS |
| | RDEPSVAAMVYPFTGDHKQKFYWGHKEILIPVFKNMADAMRKHPEVDVLINFASLRSA |
| | YDSTMETMNYAQIRTIAIIAEGIPEALTRKLIKKADQKGVTIIGPATVGGIKPGCFKI |
| | GNTGGMLDNILASKLYRPGSVAYVSRSGGMSNELNNIISRTTDGVYEGVAIGGDRYPG |
| | STFMDHVLRYQDTPGVKMIVVLGEIGGTEEYKICRGIKEGRLTKPIVCWCIGTCATMF |
| | SSEVQFGHAGACANQASETAVAKNQALKEAGVFVPRSFDELGEIIQSVYEDLVANGVI |
| | VPAQEVPPPTVPMDYSWARELGLIRKPASFMTSICDERGQELIYAGMPITEVFKEEMG |
| | IGGVLGLLWFQKRLPKYSCQFIEMCLMVTADHGPAVSGAHNTIICARAGKDLVSSLTS |
| | GLLTIGDRFGGALDAAAKMFSKAFDSGIIPMEFVNKMKKEGKLIMGIGHRVKSINNPD |
| | MRVQILKDYVRQHFPATPLLDYALEVEKITTSKKPNLILNVDGLIGVAFVDMLRNCGS |
| | FTREEADEYIDIGALNGIFVLGRSMGFIGHYLDQKRLKQGLYRHPWDDISYVLPEHMS |
| | SEQ ID NO: 253 3368 bp |
| NOV15o, | CCCGGTCCGAAGCGCGCGGATTCCACCATGTCGGCCAAGGCAATTTCAGAGCAGACGG |
| CG142427-05 | |
| DNA Sequence | GCAAAGAACTCCTTTACAAGTTCATCTGTACCACCTCAGCCATCCAGAATCGGTTCAA |
| | GTATGCTCGGGTCACTCCTGACACAGACTGGGCCCGCTTGCTGCAGGACCACCCCTGG |
| | CTGCTCAGCCAGAACTTGGTAGTCAAGCCAGACCAGCTGATCAAACGTCGTGGAAAAC |
| | TTGGTCTCGTTGGGGTCAACCTCACTCTGGATGGGGTCAAGTCCTGGCTCAAGCCACG |
| | CCTGGGACAGGAAGCCACAGTTGGCAAGGCCACAGGCTTCCTCAAGAACTTTCTGATC |
| | GAGCCCTTCGTCCCCCACAGTCAGGCTGAGGAGTTCTATGTCTGCATCTATGCCACCC |
| | GAGAAGGGGACTACGTCCTGTTCCACCACGAGGGGGGTGTGGACGTGGGTGATGTGGA |
| | CGCCAAGGCCCAGAAGCTGCTTGTTGGCGTGGATGAGAAACTGAATCCTGAGGACATC |
| | AAAAAACACCTGTTGGTCCACGCCCCTGAAGACAAGAAAGAAATTCTGGCCAGTTTTA |
| | TCTCCGGCCTCTTCAATTTCTACGAGGACTTGTACTTCACCTACCTCGAGATCAATCC |
| | CCTTGTAGTGACCAAAGATGGAGTCTATGTCCTTGACTTGGCGGCCAAGGTGGACGCC |
| | ACTGCCGACTACATCTGCAAAGTGAAGTGGGGTGACATCGAGTTCCCTCCCCCCTTCG |
| | GGCGGGAGGCATATCCAGAGGAAGCCTACATTGCAGACCTCGATGCCAAAAGTGGGGC |
| | AAGCCTGAAGCTGACCTTGCTGAACCCCAAAGGGAGGATCTGGACCATGGTGGCCGGG |
| | GGTGGCGCCTCTGTCGTGTACAGCGATACCATCTGTGATCTAGGGGGTGTCAACGAGC |
| | TGGCAAACTATGGGGAGTACTCAGGCGCCCCCAGCGAGCAGCAGACCTATGATTATGC |
| | CAAGACTATCCTCTCCCTCATGACCCGAGAGAAGCACCCAGATGGCAAGATCCTCATC |
| | ATTGGAGGCAGCATCGCAAACTTCACCAACGTGGCTGCCACGTTCAAGGGCATCGTGA |
| | GAGCAATTCGAGATTACCAGGGCCCCCTGAAGGAGCACGAAGTCACAATCTTTGTCCG |
| | AAGAGGTGGCCCCAACTATCAGGAGGGCTTACGGGTGATGGGAGAAGTCGGGAAGACC |
| | ACTGGGATCCCCATCCATGTCTTTGGCACAGAGACTCACATGACGGCCATTGTGGGCA |
| | TGGCCCTGGGCCACCGGCCCATCCCCAACCAGCCACCCACAGCGGCCCACACTGCAAA |
| | CTTCCTCCTCAACGCCAGCGGGAGCACATCGACGCCAGCCCCCAGCAGGACAGCATCT |
| | TTTTCTGAGTCCAGGGCCGATCAGGTGGCGCCTGCAAAAGAAGGCCAAGCCTGCCATGC |
| | CACAAGATTCAGTCCCAAGTCCAAGATCCCTGCAAGGAAAGAGCACCACCCTCTTCAG |
| | CCGCCACACCAAGGCCATTGTGTGGGGCATGCAGACCCGGGCCGTGCAAGGCATGCTG |
| | GACTTTGACTATGTCTGCTCCCGAGACGAGCCCTCAGTGGCTGCCATGGTCTACCCTT |
| | TCACTGGGGACCACAAGCAGAAGTTTTACTGGGGGCACAAAGAGATCCTGATCCCTGT |
| | CTTCAAGAACATGGCTGATGCCATGAGGAAGCACCCGGAGGTAGATGTGCTCATCAAC |
| | TTTGCCTCTCTCCGCTCTGCCTATGACAGCACCATGGAGACCATGAACTATGCCCAGA |
| | TCCGGACCATCGCCATCATAGCTGAAGGCATCCCTGAGGCCCTCACGAGAAAGCTGAT |
| | CAAGAAGGCGGACCAGAAGGGAGTGACCATCATCGGACCTGCCACTGTTGGAGGCATC |
| | AAGCCTGGGTGCTTTAAGATTGGCAACACAGGTGGGATGCTGGACAACATCCTGGCCT |
| | CCAAACTGTACCGCCCAGGCAGCGTGGCCTATGTCTCACGTTCCCGAGGCATGTCCAA |
| | CGAGCTCAACAATATCATCTCTCGGACCACGGATGGCGTCTATGAGGGCGTGGCCATT |
| | GGTGGGGACAGGTACCCGGGCTCCACATTCATGGATCATGTGTTACGCTATCAGGACA |
| | CTCCAGGAGTCAAAATGATTGTGGTTCTTGGAGAGATTGGGGGCACTGAGGAATATAA |
| | GATTTGCCGGGGCATCAAGGAGGGCCGCCTCACTAAGCCCATCGTCTGCTGGTGCATC |
| | GGGACGTGTGCCACCATGTTCTCCTCTGAGGTCCAGTTTGGCCATGCTGGAGCTTGTG |
| | CCAACCAGGCTTCTGAAACTGCAGTAGCCAAGAACCAGGCTTTGAAGGAAGCAGGAGT |
| | GTTTGTGCCCCGGAGCTTTGATGAGCTTGGAGAGATCATCCAGTCTGTATACGAAGAT |
| | CTCGTGGCCAATGGACTCATTGTACCTGCCCAGGAGGTGCCGCCCCCAACCGTGCCCA |
| | TGGACTACTCCTGGCCCAGGGAGCTTGGTTTGATCCGCAAACCTGCCTCGTTCATGAC |
| | CAGCATCTGCGATGAGCGAGGACAGGAGCTCATCTACGCGGGCATGCCCATCACTGAG |
| | GTCTTCAAGGAAGAGATGGGCATTGGCGGGGTCCTCGGCCTCCTCTGGTTCCAGAAAA |
| | GGTTGCCTAAGTACTCTTGCCAGTTCATTGAGATGTGTCTGATGGTGACAGCTGATCA |
| | CGGCCCAGCCGTCTCTGGAGCCCACAACACCATCATTTGTGCGCGAGCTGGGAAAGAC |
| | CTCGTCTCCAGCCTCACCTCGGGGCTGCTCACCATCGGGGATCGGTTTGGGGGTGCCT |
| | TGGATGCAGCAGCCAAGATGTTCAGTAAAGCCTTTGACAGTGGCATTATCCCCATGGA |
| | GTTTGTGAACAAGATGAAGAAGGAAGGGAAGCTGATCATGGGCATTGGTCACCGAGTG |
| | AAGTCGATAAACAACCCAGACATGCGAGTGCAGATCCTCAAAGATTACGTCAGGCAGC |
| | ACTTCCCTGCCACTCCTCTGCTCGATTATGCACTGGAAGTAGAGAAGATTACCACCTC |
| | GAAGAAGCCAAATCTTATCCTGAATGTAGATGGTCTCATCGGAGTCGCATTTGTAGAC |
| | ATGCTTAGAAACTGTGGGTCCTTTACTCGGGAGGAAGCTGATGAATATATTGACATTG |
| | GAGCCCTCAATGGCATCTTTGTGCTGGGAAGGAGTATGGGGTTCATTGGACACTATCT |
| | TGATCAGAAGAGGCTGAACCAGGGGCTGTATCGTCATCCGTGGGATGATATTTCATAT |
| | GTTCTTCCGGAACACATGAGCATGCATCATCACCACCATCACTAAGCGGCCGCTTTCG |
| | ORF Start: ATG at 28 ORF Stop: at 3331 |
| | SEQ ID NO: 254 1101 aa MW at 120838.0kD |
| NOV15o, | MSAKAISEQTGKELLYKFICTTSAIQNRFKYARVTPDTDWARLLQDHPWLLSQNLVVK |
| CG142427-05 | |
| Protein Sequence | PDQLIKRRGKLGLVGVNLTLDGVKSWLKPRLGQEATVGKATGFLKNFLIEPFVPHSQA |
| | EEFVCIYATREGDYVLFHHEGGVDVGDVDAKAQKLLVGVDEKLNPEDIKKHLLVHAP |
| | EDKKEILASFISGLFNFYEDLYFTYLEINPLVVTKDGVYVLDLAAKVDATADYI CKVK |
| | WGDIEFPPPFGREAYPEEAYIADLDAKSGASLKLTLLMPKCRIWTNVAGGCASVVYSD |
| | TICDLGGVNELANYGEYSGAPSEQQTYDYAKTILSLMTREKHPDGKILIIGGSIANFT |
| | NVAATFKGIVRAIRDYQGPLKEHEVTIPVRRGGPNYQEGLRVMGEVGKTTGIPIHVFG |
| | TETHMTAIVGMALGHRPIPNQPPTAAHTANFLLNASGSTSTPAPSRTASFSESRADEV |
| | APAKKAKPANPQDSVPSPRSLQGKSTTLFSRHTKAIVWGMQTRAVQGMLDFDYVCSRD |
| | EPSVAAIVIVYPFTCDHKQKFYWGHKEILIPVFKNMADANRKHPEVDVLINFASLRSAYD |
| | STMETMNYAQIRTIAIIAEGIPEALTRKLIKKADQKGVTIIGPATVGGIKPGCFKIGN |
| | TGGMLDNILASKLYRPGSVAYVSRSGGMSNELNNIISRTTDGVYEGVAIGGDRYPGST |
| | FMDHVLRYQDTPGVKMIVVLGEIGGTEEYKICRGIKEGRLTKPIVCWCIGTCATMFSS |
| | EVQFGHAGACANQASETAVAKNQALKEAGVFVPRSFDELGEIIQSVYEDLVANGVIVP |
| | AQEVPPPTVPMDYSWARELGLIRKPASFMTSICDERGQELIYAGMPITEVFKEEMGIG |
| | GVLGLLWFQKRLPKYSCQFIEMCLMVTADHGPAVSGAHNTIICARAGKDLVSSLTSGL |
| | LTIGDRFGGALDAAAKMFSKAFDSGIIPMEFVNKMKKEGKLIMGIGHRVKSINNPDMR |
| | VQILKDYVRQHFPATPLLDYALEVEKITTSKKPNLILNVDGLIGVAFVDMLRNCGSFT |
| | REEADEYIDIGALNGIFVLGRSMGFIGHYLDQKRLKQGLYRHPWDDISYVLPEHMSM |

Sequence comparison of the above protein sequences yields the following sequence relationships shown in Table 15B.

TABLE 15B
Comparison of NOV15a against NOV15b through NOV15o.

| | | Identities/ | |
|---|---|---|---|
| | | Similarities for | |
| Protein | NOV15a Residues/ | the Matched | |
| Sequence | Match Residues | Region | |
| NOV15b | 1 . . . 1101 | 1101/1101 | (100%) |
| | 1 . . . 1101 | 1101/1101 | (100%) |
| NOV15c | 1 . . . 1101 | 1065/1101 | (96%) |
| | 5 . . . 1072 | 1065/1101 | (96%) |
| NOV15d | 1 . . . 1101 | 1091/1101 | (99%) |
| | 5 . . . 1095 | 1091/1101 | (99%) |
| NOV15e | 1 . . . 589 | 570/610 | (93%) |
| | 5 . . . 604 | 573/610 | (93%) |
| NOV15f | 1 . . . 1101 | 1101/1101 | (100%) |
| | 5 . . . 1105 | 1101/1101 | (100%) |
| NOV15g | 1 . . . 1101 | 1091/1101 | (99%) |
| | 5 . . . 1095 | 1091/1101 | (99%) |
| NOV15h | 1 . . . 589 | 570/610 | (93%) |
| | 5 . . . 604 | 573/610 | (93%) |
| NOV15i | 1 . . . 1101 | 1101/1101 | (100%) |
| | 2 . . . 1102 | 1101/1101 | (100%) |
| NOV15j | 1 . . . 1101 | 1101/1101 | (100%) |
| | 5 . . . 1105 | 1101/1101 | (100%) |
| NOV15k | 1 . . . 1101 | 1091/1101 | (99%) |
| | 5 . . . 1095 | 1091/1101 | (99%) |
| NOV15l | 1 . . . 589 | 570/610 | (93%) |
| | 5 . . . 604 | 573/610 | (93%) |
| NOV15m | 1 . . . 1101 | 1101/1101 | (100%) |
| | 10 . . . 1110 | 1101/1101 | (100%) |
| NOV15n | 1 . . . 1101 | 1101/1101 | (100%) |
| | 3 . . . 1103 | 1101/1101 | (100%) |
| NOV15o | 1 . . . 1101 | 1101/1101 | (100%) |
| | 1 . . . 1101 | 1101/1101 | (100%) |

Further analysis of the NOV15a protein yielded the following properties shown in Table 15C.

TABLE 15C
Protein Sequence Properties NOV15a

| | |
|---|---|
| PSort | 0.8500 probability located in endoplasmic |
| analysis: | reticulum (membrane); 0.4450 probability |
| | located in microbody (peroxisome); 0.4400 |
| | probability located in plasma membrane; |
| | 0.1000 probability located in mitochondrial |
| | inner membrane |
| SignalP | No Known Signal Sequence Predicted |
| analysis: | |

A search of the NOV15a protein against the Geneseq database, a proprietary database that contains sequences published in patents and patent publication, yielded several homologous proteins shown in Table 15D.

TABLE 15D
Geneseq Results for NOV15a

| | | | Identities/ | | |
|---|---|---|---|---|---|
| | | | Similarities for | | |
| Geneseq | Protein/Organism/Length | NOV15a Residues/ | the Matched | | Expect |
| Identifier | [Patent #, Date] | Match Residues | Region | | Value |
| ABB61832 | Drosophila melanogaster | 1 . . . 1097 | 762/1099 | (69%) | 0.0 |
| | polypeptide SEQ ID NO 12288 - | 1 . . . 1083 | 895/1099 | (81%) | |
| | Drosophila melanogaster, 1086 aa. | | | | |
| | [WO200171042-A2, 27 SEP. 2001] | | | | |
| AAB56952 | Human prostate cancer antigen | 753 . . . 1101 | 347/349 | (99%) | 0.0 |
| | protein sequence SEQ ID NO: | 15 . . . 363 | 347/349 | (99%) | |
| | 1530 - Homo sapiens, 363 aa. | | | | |
| | [WO200055174-A1, 21 SEP. 2000] | | | | |
| AAY67408 | Arabidopsis ATP citrate lyase | 492 . . . 1093 | 321/602 | (53%) | 0.0 |
| | (ACL) B-2 subunit - Arabidopsis | 6 . . . 606 | 429/602 | (70%) | |
| | sp, 608 aa. [WO200000619-A2, 06 | | | | |
| | JAN. 2000] | | | | |
| AAG36247 | Arabidopsis thaliana protein | 492 . . . 1093 | 321/602 | (53%) | 0.0 |
| | fragment SEQ ID NO: 44394 - | 6 . . . 606 | 429/602 | (70%) | |
| | Arabidopsis thaliana, 681 aa. | | | | |
| | [EP1033405-A2, 06 SEP. 2000] | | | | |
| AAG36248 | Arabidopsis thaliana protein | 512 . . . 1093 | 313/582 | (53%) | 0.0 |
| | fragment SEQ ID NO: 44395 - | 1 . . . 581 | 417/582 | (70%) | |
| | Arabidopsis thaliana, 656 aa. | | | | |
| | [EP1033405-A2, 06 SEP. 2000] | | | | |

In a BLAST search of public sequence datbases, the NOV15a protein was found to have homology to the proteins shown in the BLASTP data in Table 15E.

TABLE 15E
Public BLASTP Results for NOV15a

| | | | Identities/ | | |
|---|---|---|---|---|---|
| Protein | | | Similarities for | | |
| Accession | | NOV15a Residues/ | the Matched | | Expect |
| Number | Protein/Organism/Length | Match Residues | Portion | | Value |
| P53396 | ATP-citrate (pro-S-)-lyase (EC | 1 . . . 1101 | 1100/1101 | (99%) | 0.0 |
| | 4.1.3.8) (Citrate cleavage | 1 . . . 1101 | 1101/1101 | (99%) | |
| | enzyme) - Homo sapiens (Human), | | | | |
| | 1101 aa. | | | | |
| P16638 | ATP-citrate (pro-S-)-lyase (EC | 1 . . . 1101 | 1074/1101 | (97%) | 0.0 |
| | 4.1.3.8) (Citrate cleavage | 1 . . . 1100 | 1086/1101 | (98%) | |
| | enzyme) - Rattus norvegicus | | | | |
| | (Rat), 1100 aa. | | | | |
| Q91V92 | ATP-citrate (pro-S-)-lyase (EC | 1 . . . 1101 | 1070/1101 | (97%) | 0.0 |
| | 4.1.3.8) (Citrate cleavage enzyme) - | 1 . . . 1091 | 1083/1101 | (98%) | |
| | Mus musculus (Mouse), 1091 aa. | | | | |
| S21173 | ATP citrate (pro-S)-lyase - | 1 . . . 1101 | 1078/1106 | (97%) | 0.0 |
| | human, 1105 aa. | 1 . . . 1105 | 1082/1106 | (97%) | |
| Q8VIQ1 | ATP-citrate lyase - Rattus | 250 . . . 1101 | 835/852 | (98%) | 0.0 |
| | norvegicus (Rat), 851 aa | 1 . . . 851 | 842/852 | (98%) | |
| | (fragment). | | | | |

PFam analysis predicts that the NOV15a protein contains the domains shown in the Table 15F.

TABLE 15F
Domain Analysis of NOV15a

| | | | |
|---|---|---|---|
| | | Identities/ | |
| | | Similarities for | |
| Pfam | NOV15a | the Matched | Expect |
| Domain | Match Region | Region | Value |

| | | | | |
|---|---|---|---|---|
| CoA_binding | 492 . . . 616 | 33/126 | (26%) | 1.5e−19 |
| | | 88/126 | (70%) | |
| ligase-CoA | 642 . . . 793 | 49/156 | (31%) | 3.9e−53 |
| | | 126/156 | (81%) | |

### Example 16

The NOV16 clone was analyzed, and the nucleotide and encoded polypeptide sequences are shown in Table 16A.

TABLE 16A
NOV16 Sequence Analysis

| | |
|---|---|
| | SEQ ID NO: 255 1393 bp |
| NOV16a, | CCTTCTCTTCGTGGGCTATCTACTCAGTTGATCCCTCCCTCGCTGGCTTGGCTCTGAC |
| CG142631-01 | |
| DNA Sequence | TCCTGCTCAGACCCATCACCTTTGCCGGGGAATGATGTCTGGAGAACCCCTGCACGTG |
| | AAGACCCCCATCCGTGACAGCATGGCCCTGTCCAAAATGGCCGGCACCAGCGTCTACC |
| | TCAAGATGGACAGTGCCCAGCCCTCCGGCTCCTTCAAGATCCGGGGCATTGGGCACTT |
| | CTGCAAGAGGTGGCCCAAGCAAGGCTGTGCACATTTTGTCTGCTCCTCGGCGGGCAAC |
| | GCAGGCATGGCGGCTGCATATGCGGCCAGGCAACTCGGCGTCCCCGCCACCATCGTAG |
| | TGCCCGGCACCACACCTGCTCTCACCATTGAGCGCCTCAAGAATGAAGGTGCCACATG |
| | CAAGGTGGTGGGTGAGTTATTGGATGAAGCCTTCGACCTGGCCAAGGCCCTAGCGAAG |
| | AACAACCCGGGTTGGGTCTACATTCCCCCCTTTGATGACCCCCTCATCTGGGAAGGCC |
| | ACGCTTCCATCGTGAAAGAGCTGAAGGAGACACTGTGGGAAAAGCCGGGGGCCATCGC |
| | GCTGTCAGTGGGCGGCCGCGGCCTGCTGTGTGGAGTGGTCCAGGGGCTGCAGGAGTGT |
| | GGCTGGGGGGACGTGCCTGTCATCGCCATGGAGACTTTTGGTGCCCACAGCTTCCACG |
| | CTGCCACCACCGCAGGCAAACTTGTCTCCCTGCCCAAGATCACCAGTGTTGCCAAGGC |
| | CCTGGGCGTGAAGACTGTGGGGTCTCAGGCCCTGAAGCTGTTTCAGGAACACCCCATT |
| | TTCTCTGAAGTTATCTCCGACCAGGAGGCTGTGGCCGCCATTGAGAAGTTCGTGGATG |
| | ATGAGAAGATCCTGGTGGAGCCCGCCTGGGGCGCAGCCCTGGCCGCTGTCTATAGCCA |
| | CGTGATCCAGAAGCTCCAACTGGAGGGGAATCTCCGAACCCCGCTGCCATCCCTCGTG |
| | GTCATCGTCTGCGGGGGCAGCAACATCAGCCTGGCCCAGCTGCGGGCGCTCAAGGAAC |
| | AGCTGGGCATGACAAATAGGTTGCCCAAGTGAGGACGGACCCCTTACCGATCTGTGCT |
| | CTCCTAGCCCAAGAGACCCCTGGAGGGGCTGGAGTTTATCCAGCGCCTCGTCGTATGT |
| | TTGGCTGAGCACCTGTGGCCCTGGGTGCAGGTTAACTTCTTGTTATCAGGAGCCCACT |
| | ATGCAGAGGCCAAAGGTCGGCAGCCAGCGAGGCTATGAATTGGACCTTTTTGGTATCT |
| | GTGTGACTGCTCTGTGCCCATCCTTAGCCAACTTGCTGGCGTGACAAGTGCCCACAAG |
| | TAACACACCAGGTACCCAGAGCAGGGTGGACAGGAGAGACCTGAATCACAGCAGTGAG |
| | G |
| | ORF Start: ATG at 90 ORF Stop: TGA at 1074 |
| | SEQ ID NO: 256 328 aa MW at 34702.1kD |
| NOV16a, | MMSGEPLHVKTPIRDSMALSKMAGTSVYLKMDSAQPSGSFKIRGIGHFCKRWAKQGCA |
| CG142631-01 | |
| Protein Sequence | HFVCSSAGNAGMAAAYAARQLGVPATIVVPGTTPALTIERLKNEGATCKVVGELLDEA |
| | FELAKALAKNNPGWVYIPPFDDPLIWEGHASIVKELKETLWEKPGAIALSVGGGGLLC |
| | GVVQGLQECGWGDVPVIANETPGAHSFHAATTAGKLVSLPKITSVAKALGVKTVGSQA |
| | LKLFQEHPIFSEVISDQEAVAAIEKFVDDEKILVEPAWGAALAAVYSHVIQKLQLEGN |
| | LRTPLPSLVVIVCGGSNISLAQLRALKEQLGMTNRLPK |
| | SEQ ID NO: 257 1393 bp |
| NOV16b, | CCTTCTCTTCGTGGGCTATCTACTCAGTTGATCCCTCCCTCGCTGGCTTGGCTCTGAC |
| CG142631-01 | |
| DNA Sequence | TCCTGCTCAGACCCATCACCTTTGCCGGGGAATGATGTCTGGAGAACCCCTGCACGTG |
| | AAGACCCCCATCCGTGACAGCATGGCCCTGTCCAAAATGGCCGGCACCAGCGTCTACC |
| | TCAAGATGGACAGTGCCCAGCCCTCCGGCTCCTTCAAGATCCGGGGCATTGGGCACTT |
| | CTGCAAGAGGTGGGCCAAGCAAGGCTGTGCACATTTTGTCTGCTCCTCGGCGGGCAAC |
| | GCAGGCATGGCGGCTGCATATGCGGCCAGGCAACTCGGCGTCCCCGCCACCATCGTAG |
| | TGCCCGGCACCACACCTGCTCTCACCATTGAGCGCCTCAAGAATGAAGGTGCCACATG |
| | CAAGGTGGTGGGTGAGTTATTGGATGAAGCCTTCGAGCTGGCCAAGGCCCTAGCGAAG |
| | AACAACCCGGGTTGGGTCTACATTCCCCCCTTTGATGACCCCCTCATCTGGGAAGGCC |
| | ACGCTTCCATCGTCAAAGAGCTGAAGGAGACACTGTGGGAAAAGCCGGGGGCCATCGC |
| | GCTGTCAGTGGGCGGCGGGGGCCTGCTGTGTGGAGTGGTCCAGGGGCTGCAGGAGTGT |
| | GGCTCGGGGGACGTGCCTGTCATCGCCATGGAGACTTTTGGTGCCCACAGCTTCCACG |
| | CTGCCACCACCGCAGGCAAACTTGTCTCCCTGCCCAAGATCACCAGTGTTGCCAAGGC |
| | CCTGGGCGTGAAGACTGTGGGGTCTCAGGCCCTGAAGCTGTTTCAGGAACACCCCATT |
| | TTCTCTGAAGTTATCTCGGACCAGGAGGCTGTGCCCCCCATTGAGAAGTTCGTGGATG |
| | ATGAGAAGATCCTGGTGGAGCCCGCCTGGGGCGCAGCCCTGGCCGCTGTCTATAGCCA |
| | CGTGATCCACAAGCTCCAACTGGAGGGGAATCTCCGAACCCCGCTGCCATCCCTCGTG |
| | GTCATCGTCTGCGGGGGCAGCAACATCAGCCTGGCCCAGCTGCGGGCGCTCAAGGAAC |
| | AGCTGGGCATGACAAATAGGTTGCCCAAGTGAGGACGGACCCCTTACCGATCTGTGCT |
| | CTCCTAGCCCAAGAGACCCCTGGAGGGGCTGGAGTTTATCCAGCGCCTCGTCGTATGT |
| | TTGGCTGAGCACCTGTGGCCCTGGGTGCAGGTTAACTTCTTGTTATCAGGAGCCCACT |
| | ATGCAGAGGCCAAAGGTCGGCAGCCAGCGAGGCTATGAATTGGACCTTTTTGGTATCT |
| | GTGTGACTGCTCTGTGCCCATCCTTAGCCAACTTGCTGGCGTGACAAGTGCCCACAAG |
| | TAACACACCAGGTACCCAGAGCAGGGTGGACAGGAGAGACCTGAATCACAGCAGTGAG |
| | C |
| | ORF Start: ATG at 90 ORF Stop: TGA at 1074 |
| | SEQ ID NO: 258 328 aa MW at 34702.1kD |
| NOV16b, | MMSGEPLHVKTPIRDSMALSKMAGTSVYLKMDSAQPSGSFKIRGIGHFCKRWAKQGCA |
| CG142631-01 | |
| Protein Sequence | HFVCSSAGNAGMAAAYAARQLGVPATIVVPGTTPALTIERLKNEGATCKVVGELLDEA |
| | FELAKALAKNNPGWVYIPPFDDPLIWEGHASIVKELKETLWEKPGAIALSVGGGGLLC |
| | GVVQCLQECGWGDVPVIAMETFGAHSFHAATTAGKLVSLPKITSVAKALGVKTVGSQA |
| | LKLFQEHPIFSEVISDQEAVAAIEKFVDDEKILVEPAWGAALAAVYSHVIQKLQLEGN |
| | LRTPLPSLVVIVCGGSNISLAQLRALKEQLGMTNRLPK |
| | SEQ ID NO: 259 1008 bp |
| NOV16c, | ACCATGATGTCTGGAGAACCCCTGCACGTGAAGACCCCCATCCGTGACAGCATGGCCC |
| 248494617 | |
| DNA Sequence | TGTCCAAAATGGCCGGCACCAGCGTCTACCTCAAGATGGACAGTGCCCAGCCCTCCGG |
| | CTCCTTCAAGATCCGGGGCATTGGGCACTTCTGCAAGAGGTGCGCCAAGCAAGGCTGT |
| | GCACATTTTGTCTGCTCCTCGGCGGGCAACGCAGGCATGGCGGCTGCATATGCGGCCA |
| | GGCAACTCGGCGTCCCCGCCACCATCGTGGTCCCCAGCACCACACCTGCTCTCACCAT |
| | TGAGCGCCTCAAGAATGAAGGTGCCACAGTCAAGGTGGTGGGTGAGTTATTGGATGAA |
| | GCCTTCGAGCTGGCCAAGGCCCTAGCGAAGAACAACCCGGGTTGGGTCTACATTCCCC |
| | CCTTTGATGACCCCCTCATCTGGGAAGGCCACGCTTCCATCGTGAAAGAGCTGAAGGA |
| | GACACTGTGGGAAAAGCCGGGGGCCATCGCGCTGTCAGTGGGCGGCGGGGGCCTGCTG |
| | TGTGGAGTCGTCCAGGGGCTGCAGGAGGTGGGCTGGGGGGACGTGCCTGTCATCGCCA |
| | TGGAGACTTTTGGTGCCCACAGCTTCCACGCTGCCACCACCGCAGGCAAACTTGTCTC |
| | CCTGCCCAAGATCACCAGTGTTGCCAAGGCCCTGGGCGTGAAGACTGTGGGGGCTCAG |
| | GCCCTGAAGCTGTTTCAGGAACACCCCATTTTCTCTGAAGTTATCTCGGACCAGGAGG |
| | CTGTGGCCGCCATTGAGAAGTTCGTGCATGATGAGAACATCCTGGTGGAGCCCGCCTG |
| | CGGGGCAGCCCTGGCCGCTCTCTATAGCCACGTGATCCAGAAGCTCCAACTGGAGGGG |
| | AATCTCCGAACCCCGCTGCCATCCCTCGTGGTCATCGTCTGCCGGGGCAGCAACATCA |
| | GCCTGGCCCAGCTGCGGGCGCTCAAGGAACAGCTGGGCATGACAAATAGGTTGCCCAA |
| | GCATCATCACCACCATCACTGA |
| | ORF Start: at 1 ORF Stop: TGA at 1006 |
| | SEQ ID NO: 260 335 aa MW at 35549.0kD |
| NOV16c, | TMMSGEPLHVKTPIRDSMALSKMAGTSVYLKMDSAQPSGSFKIRGIGHFCKRWAKQGC |
| 248494617 | |
| Protein Sequence | AHFVCSSAGNAGMAAAYAARQLGVPATIVVPSTTPALTIERLKNEGATVKVVGELLDE |
| | AFELAKALAKNNPGWVYIPPFDDPLIWEGHASIVKELKETLWEKPGAIALSVGGGGLL |
| | CGVVQGLQEVGWGDVPVIANETFCAHSFHAATTAGKLVSLPKITSVAKALGVKTVGAQ |
| | ALKLFQEHPIFSEVISDQEAVAAIEKFVDDEKILVEPACGAALAAVYSHVIQKLQLEG |
| | NLRTPLPSLVVIVCGGSNISLAQLRALKEQLGMTNRLPKHHHHHH |
| | SEQ ID NO: 261 988 bp |
| NOV16d, | CATGATGTCTGGAGAACCCCTGCACGTGAAGACCCCCATCCGTGACAGCATGGCCCTG |
| 228832711 | |
| DNA Sequence | TCCAAAATGGCCGGCACCAGCGTCTACCTCAAGATGGACAGTGCCCAGCCCTCCGGCT |
| | CCTTCAAGATCCGGGGCATTGGGCACTTCTGCAAGAGGTGGGCCAAGCAAGGCTGTGC |
| | ACATTTTGTCTGCTCCTCGGCGGGCAACGCAGGCATGGCGGCTGCATATGCGGCCAGG |
| | CAACTCGGCGTCCCCGCCACCATCGTGGTGCCCAGCACCACACCTGCTCTCACCATTG |
| | AGCGCCTCAAGAATGAAGGTGCCACAGTCAAGGTGGTGGGTGAGTTATTGGATGAAGC |
| | CTTCGAGCTGGCCAAGGCCCTAGCGAAGAACAACCCGGGTTGGGTCTACATTCCCCCC |
| | TTTGATGACCCCCTCATCTGGGAAGGCCACGCTTCCATCGTGAAAGACCTGAAGGAGA |
| | CACTGTGGGAAAAGCCGGGGGCCATCGCGCTGTCAGTGGGCGGCGGGGGCCTGCTGTG |
| | TGGAGTGGTCCAGGGGCTGCAGGAGGTGGGCTGGGCGGACGTGCCTGTCATCGCCATG |
| | GAGACTTTTGGTGCCCACAGCTTCCACCCTGCCACCACCGCAGGCAAACTTGTCTCCC |
| | TGCCCAAGATCACCAGTGTTGCCAAGGCCCTGGGCGTGAAGACTGTGGGGGCTCAGGC |
| | CCTGAAGCTGTTTCAGGAACACCCCATTTTCTCTGAAGTTATCTCGGACCAGGAGGCT |
| | GTGGCCGCCATTGAGAAGTTCGTGGATGATGAGAAGATCCTGGTGGAGCCCGCCTGCG |
| | GGGCAGCCCTGGCCGCTGTCTATAGCCACGTGATCCAGAAGCTCCAACTGGAGGGGAA |
| | TCTCCGAACCCCGCTGCCATCCCTCGTGGTCATCGTCTGCGGGGGCAGCAACATCAGC |
| | CTGGCCCAGCTGCGGGCGCTCAAGGAACAGCTGGGCATGACAAATAGGTTGCCCAAGT |
| | GA |
| | ORF Start: ATG at 2 ORF Stop: TGA at 986 |
| | SEQ ID NO: 262 328 aa MW at 34625.0kD |
| NOV16d, | MMSGEPLHVKTPIRDSMALSKMAGTSVYLKMDSAQPSGSFKIRGIGHFCKRwAKQGCA |
| 228832711 | |
| Protein Sequence | HFVCSSAGNAGMAAAYAARQLGVPATIVVPSTTPALTIERLKNEGATVKVVGELLDEA |
| | FELAKALAKNNPGWVYIPPPDDPLIWEGHASIVKELKETLWEKPGAIALSVGGGGLLC |
| | GVVQGLQEVGWGDVPVIAMETFGAHSFHAATTAGKLVSLPKITSVAKALGVKTVGAQA |
| | LKLFQEHPIFSEVISDQEAVAAIEKFVDDEKILVEPACGAALAAVYSHVIQKLQLEGN |
| | LRTPLPSLVVIVCGGSNISLAQLRALKEQLGMTNRLPK |
| | SEQ ID NO: 263 1035 bp |
| NOV16e, | ATGTCTGGAGAACCCCTGCACGTGAAGACCCCCATCCGTGACAGCATGGCCCTGTCCA |
| 256420310 | |
| DNA Sequence | AAATGGCCGGCACCAGCGTCTACCTCAAGATGGACAGTGCCCAGCCCTCCGGCTCCTT |
| | CAAGATCCGGGGCATTGGGCACTTCTGCAAGAGGTGGGCCAAGCAAGGCTGTGCACAT |
| | TTTGTCTGCTCCTCGGCGGGCAACGCAGGCATGGCGGCTGCATATGCGGCCAGGCAAC |
| | TCGGCGTCCCCGCCACCATCGTGGTGCCCAGCACCACACCTGCTCTCACCATTGAGCG |
| | CCTCAAGAATGAAGGTGCCACAGTCAAGGTGGTGGGTGAGTTATTGGATGAAGCCTTC |
| | GAGCTGGCCAAGGCCCTAGCGAAGAACAACCCGGGTTGGGTCTACATTCCCCCCTTTG |
| | ATGACCCCCTCATCTGCGAAGGCCACGCTTCCATCGTGAAAGAGCTGAAGGAGACACT |
| | GTGGGAAAAGCCGGGGGCCATCGCGCTGTCAGTGGGCGCCGGCGGCCTGCTCTGTGGA |
| | GTGGTCCAGGGGCTGCAGGAGGTGGGCTGGGGGGACGTGCCTGTCATCGCCATGGAGA |
| | CTTTTGGTGCCCACAGCTTCCACGCTGCCACCACCGCAGGCAAACTTGTCTCCCTGCC |
| | CAAGATCACCAGTGTTGCCAAGGCCCTGGGCGTGAAGACTGTGGGGGCTCAGGCCCTG |
| | AAGCTGTTTCAGGAACACCCCATTTTCTCTGAAGTTATCTCGGACCAGGAGGCTGTGG |
| | CCGCCATTGAGAAGTTCGTGGATGATGAGAAGATCCTGGTGGAGCCCGCCTGCGGGGC |
| | AGCCCTGGCCGCTGTCTATAGCCACGTGATCCAGAAGCTCCAACTGGAGGGGAATCTC |
| | CGAACCCCGCTGCCATCCCTCGTGGTCATCGTCTGCGGGGGCAGCAACATCAGCCTGG |
| | CCCAGCTGCGGGCGCTCAAGGAACAGCTGGGCATGACAAATAGGTTGCCCAAGCATCA |
| | TCACCACCATCACTGAGCGGCCGCACTCGAGCACCACCACCACCACCAC |
| | ORF Start: ATG at 1 ORF Stop: TGA at 1000 |
| | SEQ ID NO: 264 333 aa MW at 35316.7kD |
| NOV16e, | MSGEPLHVKTPIRDSMALSKMAGTSVYLKMDSAQPSGSFKIRGIGHFCKRWAKQGCAH |
| 256420310 | |
| Protein Sequence | FVCSSAGNAGMAAAYAARQLGVPATIVVPSTTPALTIERLKNEGATVKVVGELLDEAF |
| | ELAKALAKNNPGWVYIPPFDDPLIWEGHASIVKELKETLWEKPGAIALSVGGGGLLCG |
| | VVQGLQEVGWGDVPVIAMETFGAHSFHAATTAGKLVSLPKITSVAKALGVKTVGAQAL |
| | KLFQEHPIFSEVISDQEAVAAIEKFVDDEKILVEPACGAALAAVYSHVIQKLQLEGNL |
| | RTPLPSLVVIVCGGSNISLAQLRALKEQLGMTMRLPKHHHHHH |
| | SEQ ID NO: 265 1017 bp |
| NOV16f, | ATGTCTGGAGAACCCCTGCACGTGAAGACCCCCATCCGTGACAGCATGGCCCTGTCCA |
| 249117058 | |
| DNA Sequence | AAATGGCCGGCACCAGCGTCTACCTCAAGATGGACAGTGCCCAGCCCTCCGGCTCCTT |
| | CAAGATCCGGGGCATTGGGCACTTCTGCAAGAGGTGGGCCAAGCAAGGCTGTGCACAT |
| | TTTGTCTGCTCCTCGGCGGGCAACGCAGGCATGGCGGCTGCATATCCGGCCAGGCAAC |
| | TCGGCGTCCCCGCCACCATCGTGGTGCCCAGCACCACACCTCCTCTCACCATTGAGCG |
| | CCTCAAGAATGAAGGTGCCACAGTCAAGGTGGTGGGTGAGTTATTGGATGAAGCCTTC |
| | GAGCTGGCCAAGGCCCTAGCGAAGAACAACCCGGGTTGCGTCTACATTCCCCCCTTTG |
| | ATGACCCCCTCATCTGGGAAGGCCACGCTTCCATCGTGAAAGAGCTGAAGGAGACACT |
| | GTGGGAAAAGCCGGGGGCCATCGCGCTGTCAGTGGGCGGCGGGGGCCTGCTGTGTGGA |
| | GTGGTCCAGGGGCTGCAGGAGGTGGGCTGGGGGGACGTGCCTGTCATCGCCATGGAGA |
| | CTTTTGGTGCCCACAGCTTCCACGCTGCCACCACCGCAGGCAAACTTGTCTCCCTGCC |
| | CAAGATCACCAGTGTTGCCAAGGCCCTGGGCGTGAAGACTGTGGGGGCTCAGGCCCTG |
| | AAGCTGTTTCAGGAACACCCCATTTTCTCTGAAGTTATCTCGGACCAGGAGGCTGTGG |
| | CCGCCATTGAGAAGTTCGTGGATGATGAGAAGATCCTGGTGGAGCCCGCCTGCGGGGC |
| | AGCCCTGGCCGCTGTCTATAGCCACGTGATCCAGAAGCTCCAACTGGAGGGGAATCTC |
| | CGAACCCCGCTGCCATCCCTCGTGGTCATCGTCTGCGGGGGCAGCAACATCAGCCTGG |
| | CCCAGCTGCGGGCGCTCAAGGAACAGCTGGGCATGACAAATAGGTTGCCCAAGTGAGC |
| | GGCCGCACTCGAGCACCACCACCACCACCAC |
| | ORF Start: ATG at 1 ORF Stop: TGA at 982 |
| | SEQ ID NO: 266 327 aa MW at 34493.8kD |
| NOV16f, | MSGEPLHVKTPIRDSMALSKMAGTSVYLKMDSAQPSGSFKIRGIGHFCKRWAKQGCAH |
| 249117058 | |
| Protein Sequence | FVCSSAGNAGMAAAYAARQLGVPATIVVPSTTPALTIERLKNEGATVKVVGELLDEAF |
| | ELAKALAKNNPGWVYIPPFDDPLIWEGHASIVKELKETLWEKPGAIALSVGGGGLLCG |
| | VVQGLQEVGWGDVPVIAMETFCAHSFHAATTAGKLVSLPKITSVAKALGVKTVGAQAL |
| | KLFQEHPIFSEVISDQEAVAAIEKFVDDEKILVEPACGAALAAVYSHVIQKLQLEGNL |
| | RTPLPSLVVIVCGGSNISLAQLRALKEQLGMTNRLPK |
| | SEQ ID NO: 267 1031 bp |
| NOV16g, | CACCCGTCTCACATGGGACATCATCACCACCATCACATGTCTGGAGAACCCCTGCACG |
| 252790334 | |
| DNA Sequence | TGAAGACCCCCATCCGTGACAGCATGGCCCTGTCCAAAATGGCCGGCACCAGCGTCTA |
| | CCTCAAGATGGACAGTGCCCAGCCCTCCGGCTCCTTCAAGATCCGGGGCATTGGGCAC |
| | TTCTGCAAGAGGTGGGCCAAGCAAGGCTGTGCACATTTTGTCTGCTCCTCGGCGGGCA |
| | ACGCAGGCATGGCGGCTGCATATGCGGCCAGGCAACTCGGCGTCCCCGCCACCATCGT |
| | GGTGCCCAGCACCACACCTGCTCTCACCATTGAGCGCCTCAAGAATGAAGGTGCCACA |
| | GTCAAGGTGGTGGGTGAGTTATTGGATGAAGCCTTCGAGCTGGCCAAAGCCCTAGCGA |
| | AGAACAACCCGGGTTGGGTCTACATTCCCCCCTTTGATGACCCCCTCATCTGGGAAGG |
| | CCACGCTTCCATCGTGAAAGAGCTGAAGGAGACACTGTGGGAAAAGCCGGGGGCCATC |
| | GCGCTGTCAGTGGGCGGCGGGGGCCTGCTGTGTGGAGTGGTCCAGGGGCTGCAGGAGG |
| | TGGGCTGGGGGGACGTGCCTGTCATCGCCATGGAGACTTTTGGTGCCCACAGCTTCCA |
| | CGCTGCCACCACCGCAGGCAAACTTGTCTCCCTGCCCAAGATCACCAGTGTTGCCAAG |
| | GCCCTGGGCGTGAAGACTGTGGGGGCTCAGGCCCTGAAGCTGTTTCAGGAACACCCCA |
| | TTTTCTCTGAAGTTATCTCGGACCAGGAGGCTGTGGCCGCCATTGAGAAGTTCGTGGA |
| | TGATGAGAAGATCCTGGTGGAGCCCGCCTGCGGGGCAGCCCTGGCCGCTGTCTATAGC |
| | CACGTGATCCAGAAGCTCCAACTGGAGGGGAATCTCCGAACCCCGCTGCCATCCCTCG |
| | TGGTCATCGTCTGCGGGCGCAGCAACATCAGCCTGGCCCAGCTGCGGGCGCTCAAGGA |
| | ACAGCTGGGCATGACAAATAGGTTGCCCAAGTGAGCGGCCGCAAG |
| | ORF Start: at 1 ORF Stop: TGA at 1018 |
| | SEQ ID NO: 268 339 aa MW at 35963.4kD |
| NOV16g, | HPSHMGHHHHHHMSGEPLHVKTPIRDSMALSKMAGTSVYLKMDSAQPSGSFKIRGIGH |
| 252790334 | |
| Protein Sequence | FCKRWAKQGCAHFVCSSAGNAGMAAAYAARQLGVPATIVVPSTTPALTIERLKNEGAT |
| | VKVVGELLDEAFELAKALAKNNPGWVYIPPFDDPLIWEGHASIVKELKETLWEKPGAI |
| | ALSVGGGGLLCGVVQGLQEVGWGDVPVIAMETFGAHSFHAATTAGKLVSLPKITSVAK |
| | ALGVKTVGAQALKLFQEHPIFSEVISDQEAVAAIEKFVDDEKILVEPACGAALAAVYS |
| | HVIQKLQLEGNLRTPLPSLVVIVCGGSNISLAQLRALKEQLGMTNRLPK |
| | SEQ ID NO: 269 1036 bp |
| NOV16h, | ACATCATCACCACCATCACATGTCTGGAGAACCCCTGCACGTGAAGACCCCCATCCGT |
| 254869149 | |
| DNA Sequence | GACAGCATGGCCCTGTCCAAAATGGCCGGCACCAGCGTCTACCTCAAGATGGACAGTG |
| | CCCAGCCCTCCGGCTCCTTCAAGATCCGGGGCATTGGCCACTTCTGCAAGAGGTGGGC |
| | CAAGCAAGGCTGTGCACATTTTGTCTGCTCCTCGGCGGGCAACGCAGGCATGGCGGCT |
| | GCATATGCGGCCAGGCAACTCGGCGTCCCCGCCACCATCGTGGTGCCCAGCACCACAC |
| | CTGCTCTCACCATTGAGCGCCTCAAGAATGAAGGTGCCACAGTCAAGGTGGTGGGTGA |
| | GTTATTGGATGAAGCCTTCGAGCTGGCCAAGGCCCTAGCGAAGAACAACCCGGGTTGG |
| | GTCTACATTCCCCCCTTTGATGACCCCCTCATCTGGGAAGGCCACGCTTCCATCGTGA |
| | AAGAGCTGAAGGAGACACTGTGGGAAAAGCCGGGGGCCATCGCGCTGTCAGTGGGCGG |
| | CGGGGGCCTGCTGTGTGGAGTGGTCCAGGGGCTGCAGGAGGTGGGCTGGGGGGACGTG |
| | CCTGTCATCGCCATGGAGACTTTTGGTGCCCACAGCTTCCACGCTGCCACCACCGCAG |
| | GCAAACTTGTCTCCCTGCCCAAGATCACCAGTGTTGCCAAGGCCCTGGGCGTGAAGAC |
| | TGTGGGGGCTCAGGCCCTGAAGCTGTTTCAGGAACACCCCATTTTCTCTGAAGTTATC |
| | TCGGACCAGGAGGCTGTGCCCGCCATTGAGAAGTTCGTGGATGATGAGAAGATCCTGG |
| | TGGAGCCCGCCTGCGGGGCAGCCCTGGCCGCTGTCTATAGCCACGTGATCCAGAAGCT |
| | CCAACTGGAGGGGAATCTCCGAACCCCGCTGCCATCCCTCGTGGTCATCGTCTGCGGG |
| | GGCAGCAACATCAGCCTGGCCCAGCTGCGGGCGCTCAAGGAACAGCTGGGCATGACAA |
| | ATAGGTTGCCCAAGTGAGCGGCCGCACTCGAGCACCACCACCACCACCAC |
| | ORF Start: at 2 ORF Stop: TGA at 1001 |
| | SEQ ID NO: 270 333 aa MW at 35316.7kD |
| NOV16h, | HHHHHHMSGEPLHVKTPIRDSMALSKMAGTSVYLKMDSAQPSGSFKIRGIGHFCKRWA |
| 254869149 | |
| Protein Sequence | KQGCAHFVCSSAGNAGMAAAYAARQLGVPATIVVPSTTPALTIERLKNEGATXIKWGE |
| | LLDEAFELAKALAKNNPGWVYIPPFDDPLIWEGHASIVKELKETLWEKPGAIALSVGG |
| | GGLLCGVVQGLQEVGWGDVPVIANETFGAHSFHAATTAGKLVSLPKITSVAKALGVKT |
| | VGAQALKLFQEHPIFSEVISDQEAVAAIEKFVDDEKILVEPACGAALAAVYSHVIQKL |
| | QLEGNLRTPLPSLVVIVCGGSNISLAQLPALKEQLGMTNRLPK |
| | SEQ ID NO: 271 988 bp |
| NOV16i, | CATGATGTCTGGAGAACCCCTGCACGTGAAGACCCCCATCCGTGACAGCATGGCCCTG |
| CG142631-02 | |
| DNA Sequence | TCCAAAATGGCCGGCACCAGCGTCTACCTCAAGATGGACAGTGCCCAGCCCTCCGGCT |
| | CCTTCAAGATCCGGGGCATTGGGCACTTCTGCAAGAGGTGGGCCAAGCAACGCTGTGC |
| | ACATTTTGTCTGCTCCTCGGCGGGCAACGCAGGCATGGCGGCTGCATATGCGGCCAGG |
| | CAACTCGGCGTCCCCGCCACCATCGTGGTGCCCAGCACCACACCTGCTCTCACCATTG |
| | AGCGCCTCAAGAATGAAGGTGCCACAGTCAAGGTGGTGGGTGAGTTATTGCATGAAGC |
| | CTTCGAGCTGGCCAACGCCCTAGCGAAGAACAACCCGGGTTGGGTCTACATTCCCCCC |
| | TTTGATGACCCCCTCATCTGGGAAGGCCACGCTTCCATCGTGAAAGAGCTGAAGGAGA |
| | CACTGTGGGAAAAGCCGGGGGCCATCGCGCTGTCAGTGGGCGGCGGGGGCCTGCTGTG |
| | TGGAGTGGTCCAGGCGCTGCAGGAGGTGGGCTGGGGGGACGTGCCTGTCATCGCCATG |
| | GAGACTTTTGGTGCCCACAGCTTCCACGCTGCCACCACCGCAGGCAAACTTGTCTCCC |
| | TGCCCAAGATCACCAGTGTTGCCAAGGCCCTGGGCGTGAAGACTGTGGGGGCTCAGGC |
| | CCTGAAGCTGTTTCAGGAACACCCCATTTTCTCTGAAGTTATCTCGGACCAGGAGGCT |
| | GTGGCCGCCATTGAGAAGTTCGTGGATGATGAGAAGATCCTGGTGGAGCCCGCCTGCG |
| | GGGCAGCCCTGGCCGCTGTCTATAGCCACGTGATCCAGAAGCTCCAACTCGAGGGGAA |
| | TCTCCGAACCCCGCTGCCATCCCTCGTGGTCATCGTCTGCGGGGGCAGCAACATCAGC |
| | CTGGCCCAGCTGCGGGCGCTCAAGGAACAGCTGGGCATGACAAATAGGTTGCCCAAGT |
| | GA |
| | ORF Start: ATG at 2 ORF Stop: TGA at 986 |
| | SEQ ID NO: 272 328 aa MW at 34625.0kD |
| NOV161, | MMSGEPLHVKTPIRDSMALSKMAGTSVYLKMDSAQPSGSFKIRGIGHFCKRWAKQCCA |
| CG142631-02 | |
| Protein Sequence | HFVCSSAGNAGMAAAYAARQLGVPATIVVPSTTPALTIERLKNEGATVKVVGELLDEA |
| | FELAKALAKNNPGWVYIPPFDDPLIWEGHASIVKELKETLWEKPGAIALSVGGGGLLC |
| | GVVQGLQEVGWGDVPVIAMETFGAHSFHAATTAGKLVSLPKITSVAKALGVKTVGAQA |
| | LKLFQEHPIFSEVISDQEAVAAIEKFVDDEKILVEPACGAALAAVTSHVIQKLQLEGN |
| | LRTPLPSLVVIVCGGSNISLAQLRALKEQLGMTNRLPK |
| | SEQ ID NO: 273 1011 bp |
| NOV16j, | ACCATGGGACATCATCACCACCATCACATGTCTGGAGAACCCCTGCACGTGAAGACCC |
| CG142631-03 | |
| DNA Sequence | CCATCCGTGACAGCATGGCCCTGTCCAAAATGGCCGGCACCAGCGTCTACCTCAAGAT |
| | GGACAGTGCCCAGCCCTCCGGCTCCTTCAAGATCCGGGGCATTGGGCACTTCTGCAAG |
| | AGGTGGGCCAAGCAAGGCTGTGCACATTTTGTCTGCTCCTCGGCGGGCAACGCAGGCA |
| | TGGCGGCTGCATATGCGGCCAGGCAACTCGGCGTCCCCGCCACCATCGTGGTGCCCAG |
| | CACCACACCTGCTCTCACCATTGAGCGCCTCAAGAATGAAGGTGCCACAGTCAAGGTG |
| | GTGGGTGAGTTATTGGATGAAGCCTTCGAGCTGGCCAAGGCCCTAGCGAAGAACAACC |
| | CGGGTTGGGTCTACATTCCCCCCTTTGATGACCCCCTCATCTGGGAAGGCCACGCTTC |
| | CATCGTGAAAGAGCTGAAGGAGACACTGTGGGAAAAGCCGGGGGCCATCGCGCTGTCA |
| | GTGGGCGGCGGGGGCCTGCTGTGTGGAGTGGTCCAGGGGCTGCAGGAGGTGGGCTGGG |
| | GGGACGTGCCTGTCATCGCCATGGAGACTTTTGGTGCCCACAGCTTCCACGCTGCCAC |
| | CACCGCAGGCAAACTTGTCTCCCTGCCCAAGATCACCAGTGTTGCCAAGGCCCTGGGC |
| | GTGAAGACTGTGGGCGCTCAGGCCCTGAAGCTGTTTCAGCAACACCCCATTTTCTCTG |
| | AAGTTATCTCGGACCAGGAGGCTGTGGCCGCCATTGAGAAGTTCGTGGATGATGAGAA |
| | GATCCTGGTGGAGCCCGCCTGCGGGGCAGCCCTGGCCGCTGTCTATAGCCACGTGATC |
| | CAGAAGCTCCAACTGGAGGGGAATCTCCGAACCCCGCTGCCATCCCTCGTGGTCATCG |
| | TCTGCGGGGGCAGCAACATCAGCCTGGCCCAGCTGCGGGCGCTCAAGGAACAGCTGGG |
| | CATGACAAATAGGTTGCCCAAGTGA |
| | ORF Start: at 1 ORF Stop: TGA at 1009 |
| | SEQ ID NO: 274 336 aa MW at 35606.0kD |
| NOV16j, | TMGHHHHHHMSGEPLHVKTPIRDSMALSKMAGTSVYLKMDSAQPSGSFKIRGIGHFCK |
| CG142631-03 | |
| Protein Sequence | RWAKQGCAHFVCSSAGNAGMAAAYAARQLGVPATIVVPSTTPALTIERLKNEGATVKV |
| | VGELLDEAFELAKALAKNNPGWVYIPPFDDPLIWEGHASIVKELKETLWEKPGAIALS |
| | VGGGGLLCGVVQGLQEVGWGDVPVIAMETFGAHSFHAATTAGKLVSLPKITSVAKALG |
| | VKTVGAQALKLFQEHPIFSEVISDQEAVAAIEKFVDDEKILVEPACGAALAAVYSHVI |
| | QKLQLEGNLRTPLPSLVVIVCGGSNISLAQLRALKEQLGMTNRLPK |
| | SEQ ID NO: 275 1008 bp |
| NOV16k, | ACCATGATGTCTGGAGAACCCCTCCACGTGAAGACCCCCATCCGTGACAGCATGGCCC |
| CG142631-04 | |
| DNA Sequence | TGTCCAAAATGGCCGGCACCAGCGTCTACCTCAAGATGGACAGTGCCCAGCCCTCCGG |
| | CTCCTTCAAGATCCGGGGCATTGGGCACTTCTGCAAGAGCTGGGCCAAGCAAGGCTGT |
| | GCACATTTTGTCTGCTCCTCGGCGGGCAACGCAGGCATGGCGGCTGCATATGCGGCCA |
| | GGCAACTCGGCGTCCCCGCCACCATCGTGGTGCCCAGCACCACACCTGCTCTCACCAT |
| | TGAGCGCCTCAAGAATGAAGGTGCCACAGTCAAGGTGGTGGGTGAGTTATTGGATGAA |
| | GCCTTCGAGCTGGCCAAGGCCCTAGCGAAGAACAACCCGGGTTGGGTCTACATTCCCC |
| | CCTTTGATGACCCCCTCATCTGGGAAGGCCACGCTTCCATCGTGAAAGAGCTGAAGGA |
| | GACACTGTGGGAAAAGCCCGGGGCCATCGCGCTGTCAGTGGGCGGCGGGGGCCTGCTG |
| | TGTGGAGTGGTCCAGGGGCTGCAGGAGGTGGGCTGGGGGGACGTGCCTGTCATCGCCA |
| | TGGAGACTTTTGGTGCCCACAGCTTCCACGCTGCCACCACCGCAGGCAAACTTGTCTC |
| | CCTGCCCAAGATCACCAGTGTTGCCAAGGCCCTGGGCGTGAAGACTGTGGGGGCTCAG |
| | GCCCTGAAGCTGTTTCAGGAACACCCCATTTTCTCTGAAGTTATCTCGGACCAGGAGC |
| | CTGTGGCCGCCATTGAGAAGTTCGTGGATGATGAGAAGATCCTGGTGGAGCCCGCCTG |
| | CGGGGCAGCCCTGGCCGCTGTCTATAGCCACGTGATCCAGAAGCTCCAACTGGAGGGG |
| | AATCTCCGAACCCCGCTGCCATCCCTCGTGGTCATCGTCTGCGGGGGCAGCAACATCA |
| | GCCTGGCCCAGCTGCGGGCGCTCAAGGAACAGCTGGGCATGACAAATAGGTTGCCCAA |
| | GCATCATCACCACCATCACTGA |
| | ORF Start: at 1 ORF Stop: TGA at 1006 |
| | SEQ ID NO: 276 335 aa MW at 35549.0kD |
| NOV16k, | TMMSGEPLHVKTPIRDSMALSKMAGTSVYLKMDSAQPSGSFKIRGIGHFCKRWAKQGC |
| CG142631-04 | |
| Protein Sequence | AHFVCSSAGNAGMAAAYAARQLGVPATIVVPSTTPALTIERLKNEGATVKVVGELLDE |
| | AFFLAKALAKNNPGWVYIPPFDDPLIWEGHASIVKELKETLWEKPGAIALSVGGGGLL |
| | CGVVQGLQEVGWGDVPVIAMETFGAHSFHAATTAGKLVSLPKITSVAKALGVKTVGAQ |
| | ALKLFQEHPIFSEVISDQEAVAAIEKFVDDEKTLVEPACGAALAAVYSHTIQKLQLEG |
| | NLRTPLPSLVVIVCGGSNISLAQLRALKEQLGMTNRLPKHHHHHH |

Sequence comparison of the above protein sequences yields the following sequence relationships shown in Table 16B.

TABLE 16B
Comparison of NOV16a against NOV16b through NOV16k.

| | | Identities/ |
|---|---|---|
| | | Similarities for |
| Protein | NOV16a Residues/ | the Matched |
| Sequence | Match Residues | Region |
| NOV16b | 1 . . . 328 | 328/328 (100%) |
| | 1 . . . 328 | 328/328 (100%) |
| NOV16c | 1 . . . 328 | 323/328 (98%) |
| | 2 . . . 329 | 324/328 (98%) |
| NOV16d | 1 . . . 328 | 323/328 (98%) |
| | 1 . . . 328 | 324/328 (98%) |
| NOV16e | 2 . . . 328 | 322/327 (98%) |
| | 1 . . . 327 | 323/327 (98%) |
| NOV16f | 2 . . . 328 | 322/327 (98%) |
| | 1 . . . 327 | 323/327 (98%) |
| NOV16g | 2 . . . 328 | 322/327 (98%) |
| | 13 . . . 339 | 323/327 (98%) |
| NOV16h | 2 . . . 328 | 322/327 (98%) |
| | 7 . . . 333 | 323/327 (98%) |
| NOV16i | 1 . . . 328 | 323/328 (98%) |
| | 1 . . . 328 | 324/328 (98%) |
| NOV16j | 2 . . . 328 | 322/327 (98%) |
| | 10 . . . 336 | 323/327 (98%) |
| NOV16k | 1 . . . 328 | 323/328 (98%) |
| | 2 . . . 329 | 324/328 (98%) |

Further analysis of the NOV16a protein yielded the following properties shown in Table 16C.

TABLE 16C
Protein Sequence Properties NOV16a

| | |
|---|---|
| PSort | 0.8500 probability located in endoplasmic |
| analysis: | reticulum (membrane); 0.4400 probability |
| | located in plasma membrane; 0.1000 probability |
| | located in mitochondrial inner membrane; |
| | 0.1000 probability located in Golgi body |
| SignalP | No Known Signal Sequence Predicted |
| analysis: | |

A search of the NOV16a protein against the Geneseq database, a proprietary database that contains sequences published in patents and patent publication, yielded several homologous proteins shown in Table 16D.

TABLE 16D
Geneseq Results for NOV16a

| | | | Identities/ | |
|---|---|---|---|---|
| | | | Similarities for | |
| Geneseq | Protein/Organism/Length | NOV16a Residues/ | the Matched | Expect |
| Identifier | [Patent #, Date] | Match Residues | Region | Value |
| AAU23764 | Novel human enzyme polypeptide | 5 . . . 321 | 192/317 (60%) | e−106 |
| | #850 - Homo sapiens, 340 aa. | 23 . . . 338 | 246/317 (77%) | |
| | [WO200155301-A2, 02 AUG. | | | |
| | 2001] | | | |
| ABB89752 | Human polypeptide SEQ ID NO | 5 . . . 321 | 192/317 (60%) | e−106 |
| | 2128 - Homo sapiens, 329 aa. | 12 . . . 327 | 246/317 (77%) | |
| | [WO200190304-A2, 29 NOV. | | | |
| | 2001] | | | |
| AAM40622 | Human polypeptide SEQ ID NO | 5 . . . 321 | 192/317 (60%) | e−106 |
| | 5553 - Homo sapiens, 340 aa. | 23 . . . 338 | 246/317 (77%) | |
| | [WO200153312-A1, 26 JUL. 2001] | | | |
| AAM38836 | Human polypeptide SEQ ID NO | 5 . . . 321 | 192/317 (60%) | e−106 |
| | 1981 - Homo sapiens, 329 aa. | 12 . . . 327 | 246/317 (77%) | |
| | [WO200153312-A1, 26 JUL. 2001] | | | |
| AAU23238 | Novel human enzyme polypeptide | 5 . . . 321 | 192/317 (60%) | e−106 |
| | #324 - Homo sapiens, 340 aa. | 23 . . . 338 | 246/317 (77%) | |
| | [WO200155301-A2, 02 AUG. | | | |
| | 2001] | | | |

In a BLAST search of public sequence datbases, the NOV16a protein was found to have homology to the proteins shown in the BLASTP data in Table 16E.

TABLE 16E
Public BLASTP Results for NOV16a

| | | | Identities/ | |
|---|---|---|---|---|
| Protein | | | Similarities for | |
| Accession | | NOV16a Residues/ | the Matched | Expect |
| Number | Protein/Organism/Length | Match Residues | Portion | Value |
| P20132 | L-serine dehydratase (EC | 1 . . . 328 | 328/328 (100%) | 0.0 |
| | 4.2.1.13) (L-serine deaminase) - | 1 . . . 328 | 328/328 (100%) | |
| | Homo sapiens (Human), 328 aa. | | | |
| Q8VBT2 | Similar to serine dehydratase - | 1 . . . 328 | 270/328 (82%) | e−151 |
| | Mus musculus (Mouse), 327 aa. | 1 . . . 327 | 294/328 (89%) | |
| DWRTT | L-serine dehydratase (EC | 1 . . . 326 | 269/326 (82%) | e−151 |
| | 4.2.1.13) - rat, 327 aa. | 1 . . . 326 | 289/326 (88%) | |
| Q91X68 | Similar to serine dehydratase - | 1 . . . 313 | 260/313 (83%) | e−147 |
| | Mus musculus (Mouse), 313 aa. | 1 . . . 313 | 281/313 (89%) | |
| Q8WW81 | Hypothetical 23.0 kDa protein - | 1 . . . 217 | 214/217 (98%) | e−122 |
| | Homo sapiens (Human), 218 aa. | 1 . . . 217 | 214/217 (98%) | |

PFam analysis predicts that the NOV16a protein contains the domains shown in the Table 16F.

TABLE 16F
Domain Analysis of NOV16a

| | | Identities/ | |
|---|---|---|---|
| | | Similarities for | |
| Pfam | NOV16a | the Matched | Expect |
| Domain | Match Region | Region | Value |
| PALP | 4 . . . 298 | 97/378 (26%) | 3.8e−64 |
| | | 221/378 (58%) | |

### Example 17

The NOV17 clone was analyzed, and the nucleotide and encoded polypeptide sequences are shown in Table 17A.

TABLE 17A
NOV17 Sequence Analysis

| | |
|---|---|
| | SEQ ID NO: 277 1146 bp |
| NOV17a, | ATGAGTTGGACTGTACCTGTTGTGTGGGCCAGCCAGAGAGTGAGCTCGGCAGGAGCGA |
| CG151359-01 | |
| DNA Sequence | ATTTTCTGTGCCTGGGGATGGCCCTGTGTCCCCGTCAGGCAGCGTGCATGCCACTCAT |
| | GGGCACCTGGCTCTTCACCTCCGTGAGCAAGATGGCGACTGTGAAGAGTGAGCTTATT |
| | GAGTGCTTCACTTCCGAGGAGCCCTTTCATCACAGAAAGGTCTCCATCACAGGAACTG |
| | GATCAGTGGGCATGGCCTGCGCTACCAGCATCTTATTAAAAGGCTTGAGTGATGAACT |
| | TGCCTTTGTGGATCTTGATGAAGGCAAACTGAAAGGTGAGACAATGGATCTTCAACAT |
| | GACAGCCCTTTCATGAAAATGTCAAATATTGTTTGTAGCAAAGATTACCTTGTCACAG |
| | CAAACCCCCATCTAGTGATTATCACAGCAGGTGCACGCCGAGAAAAGGGAGAAATGCG |
| | CTTTAATTTAGTCCGGCAAAATGTGGCCATCTTCAAGTTAATGATTTCCAGTATTGTC |
| | CAGCAGAGCCCCCTCTGCAAACTAATTATTGTTTCCAATCCAGTAGATATCTTAACTT |
| | ACGTAGCCTGGAAGTTGAGTGCATTTCCCAAAAACCGTGTTATTGGAAGCGGCTGTAA |
| | TCTGGATACTGTTCGTTTTCAATTCTTCATTGGACAAAAGCTTGGTATCCACTCTGAA |
| | AGCTGCCGTGGATGGATCCTCGGAGAGCATGGAGACTCAAGTGTTCCTGTGTGGAGTG |
| | GAATGAACATAGCTGGTGTCCTTTTGAAGGATCTGAACTCTGATATAGGAACTGATAA |
| | AGATCCTGAGAAATGGAAAAATGTCCACAAAGAAGTGATTGCTAGTGCCTATGAGATT |
| | ATTGAAATGAAAAGTTCTACTTCGTGGGCCATTGGCCTATCTGGAGCTGATTTAACAG |
| | AAAGTATTTTGAAGAATCTTAGGAGAAAACATCCAGTTTCCACCATAATTAAGGGCCT |
| | CTACGGAATAAATGAAGAAGTCTTCCTCAGTATTCCTTCTTTGTTTGGAGAGAAGGGT |
| | ATTACCAACCTTATAAAGAGAAAGCTGACCCCTCAAGAGGAGGCCCATCTGAAAAAGA |
| | GTGCAAAAACACTTTGGGAAATTCAGAAGGAGCTTGAGACTTAA |
| | ORF Start: ATG at 1 ORF Stop: TAA at 1144 |
| | SEQ ID NO: 278 381 aa MW at 42104.6kD |
| NOV17a, | MSWTVPVVWASQRVSSAGANFLCLGMALCPRQAACMPLMGTWLFTSVSKMATVKSELI |
| CG151359-O1 | |
| Protein Sequence | ECFTSEEPFHHRKVSITGTGSVGMACATSILLKGLSDELAFVDLDEGKLKGETMDLQH |
| | DSPFMKMSNIVCSKDYLVTANPHLVIITAGARREKGEMRFNLVRQNVAIFKLMISSIV |
| | QQSPLCKLIIVSNPVDILTYVAWKLSAFPKNRVIGSGCNLDTVRFQFFIGQKLGIHSE |
| | SCRGWILGEHGDSSVPVWSGMNIAGVLLKDLNSDIGTDKDPEKWKNVHKEVIASAYEI |
| | IEMKSSTSWAIGLSGADLTESILKNLRRKHPVSTIIKGLYGINEEVFLSIPSLFGEKG |
| | ITNLIKRKLTPEEEAHLKKSAKTLWEIQKELET |

Further analysis of the NOV17a protein yielded the following properties shown in Table 17B.

TABLE 17B
Protein Sequence Properties NOV17a

| | |
|---|---|
| PSort | 0.6736 probability located in nucleus; |
| analysis: | 0.5701 probability located in mitochondrial |
| | matrix space; 0.3952 probability located in |
| | microbody (peroxisome); 0.2847 probability |
| | located in mitochondrial inner membrane |
| SignalP | Cleavage site between residues 49 and 50 |
| analysis: | |

A search of the NOV17a protein against the Geneseq database, a proprietary database that contains sequences published in patents and patent publication, yielded several homologous proteins shown in Table 17C.

TABLE 17C
Geneseq Results for NOV17a

| | | | Identities/ | |
|---|---|---|---|---|
| | | | Similarities for | |
| Geneseq | Protein/Organism/Length | NOV17a Residues/ | the Matched | Expect |
| Identifier | [Patent #, Date] | Match Residues | Region | Value |
| AAU11432 | Human testicular lactate | 1 . . . 380 | 328/380 (86%) | 0.0 |
| | dehydrogenase A - Homo sapiens, | 1 . . . 380 | 344/380 (90%) | |
| | 381 aa. [CN1313342-A, 19 SEP. | | | |
| | 2001] | | | |
| AAG89135 | Human secreted protein, SEQ ID | 1 . . . 380 | 328/380 (86%) | 0.0 |
| | NO: 255 - Homo sapiens, 381 aa. | 1 . . . 380 | 344/380 (90%) | |
| | [WO200142451-A2, 14 JUN. 2001] | | | |
| AAY36058 | Extended human secreted protein | 1 . . . 380 | 321/380 (84%) | 0.0 |
| | sequence, SEQ ID NO. 443 - Homo | 1 . . . 380 | 336/380 (87%) | |
| | sapiens, 381 aa. [WO9931236-A2, | | | |
| | 24 JUN. 1999] | | | |
| AAM42058 | Human polypeptide SEQ ID NO | 44 . . . 380 | 221/337 (65%) | e−128 |
| | 6989 - Homo sapiens, 372 aa. | 35 . . . 371 | 271/337 (79%) | |
| | [WO200153312-A1, 26 JUL. 2001] | | | |
| AAM40272 | Human polypeptide SEQ ID NO | 50 . . . 380 | 218/331 (65%) | e−127 |
| | 3417 - Homo sapiens, 332 aa. | 1 . . . 331 | 268/331 (80%) | |
| | [WO200153312-A1, 26 JUL. 2001] | | | |

In a BLAST search of public sequence datbases, the NOV17a protein was found to have homology to the proteins shown in the BLASTP data in Table 17D.

TABLE 17D
Public BLASTP Results for NOV17a

| | | | Identities/ | |
|---|---|---|---|---|
| Protein | | | Similarities for | |
| Accession | | NOV17a Residues/ | the Matched | Expect |
| Number | Protein/Organism/Length | Match Residues | Portion | Value |
| Q9BYZ2 | L-lactate dehydrogenase A-like (EC | 1 . . . 380 | 328/380 (86%) | 0.0 |
| | 1.1.1.27) - Homo sapiens (Human), | 1 . . . 380 | 344/380 (90%) | |
| | 381 aa. | | | |
| Q96LI2 | CDNA FLJ25463 fis, clone | 1 . . . 380 | 325/380 (85%) | 0.0 |
| | TST09242 (Lactate dehydrogenase | 1 . . . 380 | 342/380 (89%) | |
| | A-like) - Homo sapiens (Human), | | | |
| | 381 aa. | | | |
| DEMSLM | L-lactate dehydrogenase (EC | 50 . . . 380 | 220/331 (66%) | e−129 |
| | 1.1.1.27) chain M - mouse, 332 aa. | 1 . . . 331 | 271/331 (81%) | |
| P06151 | L-lactate dehydrogenase A chain (EC | 51 . . . 380 | 219/330 (66%) | e−128 |
| | 1.1.1.27) (LDH-A) (LDH muscle | 1 . . . 330 | 270/330 (81%) | |
| | subunit) (LDH-M) - Mus musculus | | | |
| | (Mouse), 331 aa. | | | |
| Q9XT87 | L-lactate dehydrogenase A chain (EC | 52 . . . 380 | 219/329 (66%) | e−127 |
| | 1.1.1.27) (LDH-A) (LDH muscle | 2 . . . 330 | 269/329 (81%) | |
| | subunit) (LDH-M) - Monodelphis | | | |
| | domestica (Short-tailed grey | | | |
| | opossum), 331 aa. | | | |

PFam analysis predicts that the NOV17a protein contains the domains shown in the Table 17E.

TABLE 17E
Domain Analysis of NOV17a

| | | Identities/ | |
|---|---|---|---|
| | | Similarities for | |
| Pfam | NOV17a | the Matched | Expect |
| Domain | Match Region | Region | Value |
| ldh | 67 . . . 210 | 63/156 (40%) | 9.1e−55 |
| | | 120/156 (77%) | |
| ldh_C | 212 . . . 380 | 68/179 (38%) | 4.4e−67 |
| | | 148/179 (83%) | |

### Example 18

The NOV18 clone was analyzed, and the nucleotide and encoded polypeptide sequences are shown in Table 18A.

TABLE 18A
NOV18 Sequence Analysis

| | |
|---|---|
| | SEQ ID NO: 279 1015 bp |
| NOV18a, | CTCTGCTGCTTTAGTTTCGGAGTGTTTGGCGACGGGGCAGCGCGAGATGTGGAGGCTC |
| CG152227-01 | |
| DNA Sequence | ATGTCGAGGTTTAATGCATTCAAAAGGACTAATACCATACTGCACCATTTGAGAATGT |
| | CCAAGCACACAGATGCAGCAGAAGAGGTGCTATTGGAAAAAAAAGGTTGCGCGGGAGT |
| | CATAACACTAAACAGACCAAAGTTCCTCAATGCACTGACTCTTAATATGATTCGGCAG |
| | ATTTATCCACAGCTAAAGAAGTGCGAACAAGATCCTGAAACTTTCCTGATCATTATAA |
| | AGGGAGCAGGAGGAAAGGCTTTCTGTGCCGGGGGTGATATCAGAGTGATCTCGGAAGC |
| | TGAAAAGGCAAAACAGAAOATAGCTCCAGTTTTCTTCAGAGAAGAATATATGCTGAAT |
| | AATGCTGTTGGTTCTTGCCAGAAACCTTATGTTGCACTTATTCATGGAATTACAATGG |
| | GTGGGGGAGTTGGTCTCTCAGTCCATGGGCAATTTCGAGTGGCTACAGAAAAGTGTCT |
| | TTTTGCTATGCCAGAAACTGCAATAGGACTGTTCCCTGATGTGGGTGGAGGTTATTTC |
| | TTGCCACGACTCCAAGGAAAACTTGGTTACTTCCTTGCATTAACAGGATTCAGACTAA |
| | AAGGAAGAGATGTGTACAGAGCAGGAATTGCTACACACTTTGTAGATTCTGAAAAGTT |
| | GGCCATGTTAGAGGAAGATTTGTTAGCCTTGAAATCTCCTTCAAAAGAAAATATTGCA |
| | TCTGTCTTAGAAAATTACCATACAGAGTCTAAGATTGATCGAGACAAGTCTTTTATAC |
| | TTGAAGACCAGAGTCCAAAATGGAAACCAGCTGATCTAAAAGAAGCTACTGAGGAAGA |
| | TTTGAATAATCACTTTAAGTCTTTGGGAAGCAGTGATTTGAAATTTTGAGGTGACAGG |
| | CTTTTAAGGTATATTTTGTAGCATGGGTTGGCAATCTACAGCATGTGGGCCAAATCCA |
| | GCCTGCTGCCTGTTTTTATATACCCTGTA |
| | ORF Start: ATG at 47 ORF Stop: TGA at 917 |
| | SEQ ID NO: 280 290 aa MW at 32497.3kD |
| NOV18a, | MWRLMSRFNAFKRTNTILHHLRMSKHTDAAEEVLLEKKGCAGVITLNRPKFLNALTLN |
| CG152227-01 | |
| Protein Sequence | MIRQIYPQLKKWEQDPETFLIIIKGAGGKAFCAGGDIRVISEAEKAKQKIAPVFFREE |
| | YMLNNAVGSCQKPYVALIHGITMGGGVGLSVHGQFRVATEKCLFANPETAIGLFPDVG |
| | GGYFLPRLQGKLGYFLALTGFRLKGRDVYRAGIATHFVDSEKLAMLEEDLLALKSPSK |
| | ENIASVLENYHTESKIDRDKSFILEDQSPKWKPADLKEATEEDLNNHFKSLGSSDLKF |
| | SEQ ID NO: 281 1311 bp |
| NOV18b, | AGTCCGGGAGATTCTCCCTCTGCTGCTTTAGTTTCGGAGTGTTTGGCGACGGGGCAGC |
| CG152227-02 | |
| DNA Sequence | GCGAGATGTGGAGGCTCATGTCGAGGTTTAATGCATTCAAAAGGACTAATACCATACT |
| | GCACCATTTGAGAATGTCCAAGCACACAGATGCAGCAGAAGAGGTGCTATTGGAAAAA |
| | AAAGGTTGCGCGGGAGTCATAACACTAAACAGACCAAAGTTCCTCAATGCACTGACTC |
| | TTAATATGATTCGGCAGATTTATCCACAGCTAAAGAAGTGGGAACAAGATCCTGAAAC |
| | TTTCGTGATCATTATAAAGGGAGCAGGAGGAAAGGCTTTCTGTGCCGGGGGTGATATC |
| | AGAGTGATCTCGGAAGCTGAAAAGGCAAAACAGAAGATAGCTCCAGTTTTCTTCAGAG |
| | AAGAATATATGCTGAATAATGCTGTTGGTTCTTGCCAGAAACCTTATGTTGCACTTAT |
| | TCATGGAATTACAATGGGTGGGGGAGTTGGTCTCTCAGTCCATGGGCAATTTCGAGTG |
| | GCTACAGAAAAGTGTCTTTTTGCTATGCCAGAAACTGCAATAGGACTGTTCCCTGATG |
| | TGGGTGGAGGTTATTTCTTTGCCACGACTCCAAGGAAAACTTGGTTACTTCCTTGCAT |
| | TAACGGATTCAGACTAAAAGGAAGAGATGTGTACAGAGCAGGAATTGCTACACACTTT |
| | GTAGATTCTGAAAAGTTGGCCATGTTAGAGGAAGATTTGTTAGCCTTGAAATCTCCTT |
| | CAAAAGAAAATATTGCATCTGTCTTAGAAAATTACCATACAGAGTCTAAGATTGATCG |
| | AGACAAGTCTTTTATACTTGAGGAACACATGGACAAAATAAACAGTTGTTTTTCAGCC |
| | AATACTGTGGAAGAAATTATTGAAAACTTACAGCAAGATGGTTCATCTTTTGCCCTAG |
| | AGCAATTGAAGGTAATTAATAAAATGTCTCCAACATCTCTAAAGATCACACTAAGGCA |
| | ACTCATGGAGGGGTCTTCAAAGACCTTGCAAGAAGTACTAACTATGGAGTATCGGCTA |
| | AGTCAAGCTTGTATGAGAGGTCATGACTTTCATGAACGCGTTAGAGCTGTTTTAATTG |
| | ATAAAGACCAGAGTCCAAAATGGAAACCAGCTGATCTAAAAGAAGTTACTGAGGAAGA |
| | TTTGAATAATCACTTTAAGTCTTTGGGAAGCAGTGATTTGAAATTTTGAGGTGACAGG |
| | CTTTTAAGGTATATTTTGTAGCATGGGTTGGCAATCTACAGCATGTGGGCCAAATCCA |
| | GCCTGCTGCCTGTTTTTATATACCCTGTAAGCAAG |
| | ORF Start: ATG at 64 ORF Stop: TGA at 1207 |
| | SEQ ID NO: 282 381 aa MW at 42907.1kD |
| NOV18b, | MWRLMSRFNAFKRTNTILHHLRMSKHTDAAEEVLLEKKGCAGVITLNRPKFLNALTLN |
| CG152227-02 | |
| Protein Sequence | MIRQIYPQLKKWEQDPETFVIIIKGAGGKAFCAGGDIRVISEAEKAKQKIAPVFFREE |
| | YMLNNAVGSCQKPYVALIHGITMGGGVGLSVHGQFRVATEKCLFAMPETAIGLFPDVG |
| | GGYFFATTPRKTWLLPCINGFRLKGRDVYRAGIATHFVDSEKLANLEEDLLALKSPSK |
| | ENIASVLENYHTESKIDRDKSFILEEHMDKINSCFSANTVEEIIENLQQDGSSFALEQ |
| | LKVINKNSPTSLKITLRQLMEGSSKTLQEVLTMEYRLSQACMRGHDFHEGVRAVLIDK |
| | DQSPKWKPADLKEVTEEDLNNHFKSLGSSDLKF |

Sequence comparison of the above protein sequences yields the following sequence relationships shown in Table 18B.

TABLE 18B
Comparison of NOV18a against NOV18b.

| | | Identities/ |
|---|---|---|
| | | Similarities for |
| Protein | NOV18a Residues/ | the Matched |
| Sequence | Match Residues | Region |
| NOV18b | 1 . . . 278 | 246/278 (88%) |
| | 1 . . . 278 | 250/278 (89%) |

Further analysis of the NOV18a protein yielded the following properties shown in Table 18C.

TABLE 18C
Protein Sequence Properties NOV18a

| | |
|---|---|
| PSort | 0.6784 probability located in mitochondrial matrix space; |
| analysis: | 0.3893 probability located in microbody (peroxisome); |
| | 0.3672 probability located in mitochondrial inner |
| | membrane; 0.3672 probability located in mitochondrial |
| | intermembrane space |
| SignalP | No Known Signal Sequence Predicted |
| analysis: | |

A search of the NOV18a protein against the Geneseq database, a proprietary database that contains sequences published in patents and patent publication, yielded several homologous proteins shown in Table 18D.

TABLE 18D
Geneseq Results for NOV18a

| | | | Identities/ | |
|---|---|---|---|---|
| | | | Similarities for | |
| Geneseq | Protein/Organism/Length | NOV18a Residues/ | the Matched | Expect |
| Identifier | [Patent #, Date] | Match Residues | Region | Value |
| AAW81135 | Human 3-hydroxyisobutyryl- | 1 . . . 278 | 259/278 (93%) | e−147 |
| | coenzyme A hydrolase - Homo | 1 . . . 278 | 261/278 (93%) | |
| | sapiens, 381 aa. [WO9851782-A2, | | | |
| | 19 NOV. 1998] | | | |
| AAG75795 | Human colon cancer antigen protein | 2 . . . 176 | 158/175 (90%) | 1e−86 |
| | SEQ ID NO: 6559 - Homo sapiens, | 1 . . . 175 | 159/175 (90%) | |
| | 178 aa. [WO200122920-A2, 05 | | | |
| | APR. 2001] | | | |
| ABB61217 | Drosophila melanogaster | 29 . . . 278 | 131/253 (51%) | 2e−63 |
| | polypeptide SEQ ID NO 10443 - | 8 . . . 250 | 171/253 (66%) | |
| | Drosophila melanogaster, 351 aa. | | | |
| | [WO200171042-A2, 27 SEP. 2001] | | | |
| AAG23865 | Arabidopsis thaliana protein | 23 . . . 254 | 98/233 (42%) | 9e−50 |
| | fragment SEQ ID NO: 27329 - | 1 . . . 232 | 148/233 (63%) | |
| | Arabidopsis thaliana, 378 aa. | | | |
| | [EP1033405-A2, 06 SEP. 2000] | | | |
| AAG23866 | Arabidopsis thaliana protein | 32 . . . 254 | 97/224 (43%) | 1e−49 |
| | fragment SEQ ID NO: 27330 - | 6 . . . 228 | 145/224 (64%) | |
| | Arabidopsis thaliana, 374 aa. | | | |
| | [EP1033405-A2, 06 SEP. 2000] | | | |

In a BLAST search of public sequence datbases, the NOV18a protein was found to have homology to the proteins shown in the BLASTP data in Table 18E.

TABLE 18E
Public BLASTP Results for NOV18a

| | | | Identities/ | |
|---|---|---|---|---|
| Protein | | | Similarities for | |
| Accession | | NOV18a Residues/ | the Matched | Expect |
| Number | Protein/Organism/Length | Match Residues | Portion | Value |
| Q9BS94 | Similar to 3-hydroxyisobutyryl- | 1 . . . 278 | 261/278 (93%) | e−148 |
| | coenzyme A hydrolase - Homo | 1 . . . 278 | 263/278 (93%) | |
| | sapiens (Human), 333 aa. | | | |
| Q92931 | 3-hydroxyisobutyryl-coenzyme A | 1 . . . 278 | 246/278 (88%) | e−138 |
| | hydrolase - Homo sapiens | 1 . . . 278 | 250/278 (89%) | |
| | (Human), 381 aa. | | | |
| Q8QZS1 | Similar to 3-hydroxyisobutyryl- | 2 . . . 278 | 207/277 (74%) | e−118 |
| | coenzyme A hydrolase - Mus | 7 . . . 282 | 238/277 (85%) | |
| | musculus (Mouse), 385 aa. | | | |
| Q9VF79 | CG5044 protein - Drosophila | 29 . . . 278 | 131/253 (51%) | 6e−63 |
| | melanogaster (Fruit fly), 351 aa. | 8 . . . 250 | 171/253 (66%) | |
| Q960K8 | LD47223p - Drosophila | 29 . . . 278 | 131/253 (51%) | 6e−63 |
| | melanogaster (Fruit fly), 385 aa. | 42 . . . 284 | 171/253 (66%) | |

PFam analysis predicts that the NOV18a protein contains the domains shown in the Table 18F.

TABLE 18F
Domain Analysis of NOV18a

| | | Identities/ | |
|---|---|---|---|
| | | Similarities for | |
| Pfam | NOV18a | the Matched | Expect |
| Domain | Match Region | Region | Value |
| ECH | 42 . . . 213 | 54/176 (31%) | 2.3e−17 |
| | | 112/176 (64%) | |

### Example 19

The NOV19 clone was analyzed, and the nucleotide and encoded polypeptide sequences are shown in Table 19A.

TABLE 19A
NOV19 Sequence Analysis

| | |
|---|---|
| | SEQ ID NO: 283 1935 bp |
| NOV19a, | GTGCTGGCTTGCCCTGCAAATTGTGTCTGCAGCAAGACTGAGATCAATTGCCGGCGGC |
| CG152392-01 | |
| DNA Sequence | CGCACGATGCCAACCTCTTCCCCCTCCTGGAAGGGCAGGATTCAGGGAACAGCAATGG |
| | GAACGCCAGTATCAACATCACGGACATCTCAAGGAATATCACTTCCATACACATAGAG |
| | AACTGGCGCAGTCTTCACACGCTCAACGCCGTGGACATGCAGCTCTACACCGGACTTC |
| | AAAAGCTGACCATCAAGAACTCAGGACTTCGGAGCATTCAGCCCAGAGCCTTTGCCAA |
| | GAACCCCCATTTGCGTTATATAAACCTGTCAAGTAACCGGCTCACCACACTCTCGTGG |
| | CAGCTCTTCCAGACGCTCAGTCTTCGGGAATTGCAGTTGGAGCAGAACTTTTTCAACT |
| | GCAGCTGTGACATCCGCTCGATGCAGCTCTGGCAGGAGCAGGGGGAGGCCAAGCTCAA |
| | CAGCCAGAACCTCTACTGCATCAATGCTGATGCCTCCCAGCTTCCTCTCTTCCGCATG |
| | AACATCAGTCAGTGTGACCTTCCTGAGATCAGCGTGAGCCACGTCAACCTGACCGTAC |
| | GAGAGGGTGACAATGCTGTTATCACTTGCAATGGCTCTGGATCACCCCTTCCTGATGT |
| | GGACTGGATAGTCACTGGGCTGCAGTCCATCAACACTCACCAGACCAATCTGAACTGG |
| | ACCAATCTTCATGCCATCAACTTGACGCTGGTGAATGTGACGAGTGAGGACAATGGCT |
| | TCACCCTGACGTGCATTGCAGAGAACGTGGTGGGCATGAGCAATGCCAGTGTTGCCCT |
| | CACTGTCTACTATCCCCCACGTGTGGTGAGCCTGGAGGAGCCTGAGCTGCGCCTGGAG |
| | CACTGCATCGAGTTTGTCGTGCGTGGCAACCCCCCACCAACGCTGCACTGGCTGCACA |
| | ATGGGCAGCCTCTGCGGGAGTCCAAGATCATCCATGTGGAATACTACCAAGAGGGAGA |
| | GATTTCCGAGGGCTGCCTGCTCTTCAACAAGCCCACCCACTACAACAATGGCAACTAT |
| | ACCCTCATTGCCAAAAACCCACTGGGCACAGCCAACCAGACCATCAATGGCCACTTCC |
| | TCAAGGAGCCCTTTCCAGTTGACGAAGTGAGTCCCACACCTCCTATCACTGTGACCCA |
| | CAAACCAGAAGAAGACACTTTTGGGGTATCCATAGCAGTTGGACTTGCTGCTTTTGCC |
| | TGTGTCCTGTTGGTGGTTGTCTTCGTCATGATCAACAAATATGGTCGACGGTCCAAAT |
| | TTGCAATGAACGGTCCCGTGGCTGTCATCAGTGGTGAGGAGGACTCAGCCAGCCCACT |
| | GCACCACATCAACCACGGCATCACCACGCCCTCGTCACTGGATGCGGGGCCCGACACT |
| | GTGGTCATTGGCATGACTCGCATCCCTGTCATTGAGAACCCCCAGTACTTCCGTCAGG |
| | GACACAACTGCCACAAGCCGGACACGTGGGTCTTTTCAAACATAGACAATCATGGGAT |
| | ATTAAACTTGAAGGACAATAGAGATCATCTAGTCCCATCAACTCACTATATATATGAG |
| | GAACCTGAGGTCCAGAGTGGGGAAGTGTCTTACCCAAGGTCACATGGTTTCAGAGAAA |
| | TTATGTTGAATCCAATAAGCCTTCCCGGACATTCCAAGCCTCTTAACCATGGCATCTA |
| | TGTTGAGGATGTCAATGTTTATTTCAGCAAAGGACGTCATGGCTTTTAAAAACTCCTT |
| | TTAAGCCTCCTTGTTTTGATGTCACCTTGGTAGGCTGGGCCCTCTGAGAGGTTGGAAG |
| | CTCTAGGCATTGTTCTCTTTGGATCCAGGGATGCTAAGTAGAAACTGCATGAGCCACC |
| | AGTGCCCCGGCACCCTTTAACACCACCAGATGGGTGTTTTCCCCCATCCACCACTGGC |
| | AGGGCTTGCCAGGAGTAAGAG |
| | ORF Start: at 1 ORF Stop: TAA at 1729 |
| | SEQ ID NO: 284 576 aa MW at 64294.1kD |
| NOV19a, | VLACPANCVCSKTEINCRRPDDGNLFPLLEGQDSGNSMGNASINITDISRNITSIHIE |
| CG152392-01 | |
| Protein Sequence | NWRSLHTLNAVDMELYTGLQKITIKNSGLRSIQPRAFAKNPHLRYINLSSNRLTTLSW |
| | QLFQTLSLRELQLEQNFFNCSCDIRWMQLWQEQGEAKLNSQNLYCINADGSQLPLFRN |
| | NISQCDLPETSVSHVNLTVREGDNAVITCNGSGSPLPDVDWIVTGLQSINTHQTNLNW |
| | TNVHAINLTLVNVTSEDMGFTLTCIAENVVGMSNASVALTVYYPPRVVSLEEPELRLE |
| | HCIEFVVRGNPPPTLHWLHNGQPLRESKIIHVEYYQEGEISEGCLLFNKPTHYNNGNY |
| | TLIAKNPLGTANQTINGHFLKEPFPVDEVSPTPPITVTHKPEEDTFGVSIAVGLAAFA |
| | CVLLVVVFVMINKYGRRSKFGMKGPVAVISGEEDSASPLHHINHGITTPSSLDAGPDT |
| | VVIGMTRIPVIENPQYFRQGHNCHKPDTWVFSNIDNHGILNLKDNRDHLVPSTHYIYE |
| | EPEVQSGEVSYPRSHGFREIMLNPISLPGHSKPLNHGIYVEDVNVYFSKGRHGF |

Further analysis of the NOV19a protein yielded the following properties shown in Table 19B.

TABLE 19B
Protein Sequence Properties NOV19a

| | |
|---|---|
| PSort | 0.8357 probability located in mitochondrial inner membrane; |
| analysis: | 0.8200 probability located in plasma membrane; 0.3000 |
| | probability located in microbody (peroxisome); 0.2000 |
| | probability located in endoplasmic reticulum (membrane) |
| SignalP | No Known Signal Sequence Predicted |
| analysis: | |

A search of the NOV19a protein against the Geneseq database, a proprietary database that contains sequences published in patents and patent publication, yielded several homologous proteins shown in Table 19C.

TABLE 19C
Geneseq Results for NOV19a

| | | | Identities/ | |
|---|---|---|---|---|
| | | | Similarities for | |
| Geneseq | Protein/Organism/Length | NOV19a Residues/ | the Matched | Expect |
| Identifier | [Patent #, Date] | Match Residues | Region | Value |
| AAY51602 | Human truncated trkC receptor | 1 . . . 576 | 573/584 (98%) | 0.0 |
| | protein - Homo sapiens, 612 aa. | 29 . . . 612 | 575/584 (98%) | |
| | [US6027927-A, 22 FEB. 2000] | | | |
| AAR81627 | Human trkC receptor protein | 1 . . . 494 | 490/494 (99%) | 0.0 |
| | mutant - Homo sapiens, 830 aa. | 29 . . . 521 | 493/494 (99%) | |
| | [WO9525795-A1, 28 SEP. 1995] | | | |
| AAY06595 | Neurotrophin-3 receptor TrkC - | 1 . . . 494 | 491/502 (97%) | 0.0 |
| | Homo sapiens, 825 aa. | 29 . . . 530 | 493/502 (97%) | |
| | [WO9940103-A1, 12 AUG. 1999] | | | |
| AAM50853 | Human receptor tyrosine kinase | 1 . . . 494 | 490/502 (97%) | 0.0 |
| | TrkC - Homo sapiens, 839 aa. | 29 . . . 530 | 493/502 (97%) | |
| | [WO200203071-A2, 10 JAN. | | | |
| | 2002] | | | |
| AAY51601 | Human trkC receptor protein - | 1 . . . 494 | 490/502 (97%) | 0.0 |
| | Homo sapiens, 839 aa. | 29 . . . 530 | 493/502 (97%) | |
| | [US6027927-A, 22 FEB. 2000] | | | |

In a BLAST search of public sequence datbases, the NOV19a protein was found to have homology to the proteins shown in the BLASTP data in Table 19D.

TABLE 19D
Public BLASTP Results for NOV19a

| | | | Identities/ | |
|---|---|---|---|---|
| Protein | | | Similarities for | |
| Accession | | NOV19a Residues/ | the Matched | Expect |
| Number | Protein/Organism/Length | Match Residues | Portion | Value |
| Q96CY4 | Hypothetical 68.5 kDa protein - | 1 . . . 576 | 574/584 (98%) | 0.0 |
| | Homo sapiens (Human), 612 aa. | 29 . . . 612 | 575/584 (98%) | |
| I73633 | gene trkC protein - human, 612 aa. | 1 . . . 576 | 573/584 (98%) | 0.0 |
| | | 29 . . . 612 | 575/584 (98%) | |
| Q9Z2P9 | Neurotrophin-3 receptor non- | 1 . . . 576 | 553/584 (94%) | 0.0 |
| | catalytic isoform 2 - Mus | 29 . . . 612 | 568/584 (96%) | |
| | musculus (Mouse), 612 aa. | | | |
| A55178 | neurotrophin receptor trkC | 1 . . . 494 | 491/502 (97%) | 0.0 |
| | precursor - human, 825 aa. | 29 . . . 530 | 493/502 (97%) | |
| O75682 | TRKC protein - Homo sapiens | 1 . . . 494 | 491/502 (97%) | 0.0 |
| | (Human), 839 aa. | 29 . . . 530 | 493/502 (97%) | |

PFam analysis predicts that the NOV19a protein contains the domains shown in the Table 19E.

TABLE 19E
Domain Analysis of NOV19a

| | | Identities/ | |
|---|---|---|---|
| | | Similarities for | |
| Pfam | NOV19a | the Matched | Expect |
| Domain | Match Region | Region | Value |
| LRRNT | 3 . . . 30 | 9/31 (29%) | 0.00013 |
| | | 23/31 (74%) | |
| LRR | 100 . . . 123 | 8/25 (32%) | 0.0043 |
| | | 22/25 (88%) | |
| LRRCT | 132 . . . 180 | 13/54 (24%) | 2.4e−10 |
| | | 40/54 (74%) | |
| ig | 196 . . . 258 | 20/65 (31%) | 4.8e−07 |
| | | 43/65 (66%) | |

### Example 20

The NOV20 clone was analyzed, and the nucleotide and encoded polypeptide sequences are shown in Table 20A.

TABLE 20A
NOV20 Sequence Analysis

| | |
|---|---|
| | SEQ ID NO: 285 1201 bp |
| NOV2Oa, | GCCCTTCTGGCAGGAAGAGGAAGATGTCTGTGCTCAGGCGGATGATGCCGGTTTCCAA |
| CG152453-01 | |
| DNA Sequence | TCGCTCTCTCCTCGCCTTCATCTTCTTCTTCTCCCTCTCTTCGTCCTGTCTGTACTTC |
| | ATCTATGTGGCCCCAGGCATCGCCAACACATATCTCTTTATGGTACAAGCTCGAGGTA |
| | TAATGTTGAGAGAAAATGTGAAAACAATAGGTCATATGATCAGGCTGTACACAAATAA |
| | AAACAGTACGCTCAACGGTACAGATTATCCCGAAGGCAATAATTCAAGTGATTATCTT |
| | GTTCAAACAACAACGTATCTCCCGGAAAACTTCACATACTCACCATACCTCCCCTGTC |
| | CAGAAAAGCTGCCTTATATGCGAGGATTCCTCAATGTCAATGTAACCGAAGTCAGTTT |
| | TGATGAAATTCATCAACTCTTCTCCAACOATTTAGATATTGAGCCAGGGGGTCATTGG |
| | AGGCCAAAAGACTGTAAACCCAGATGGAAGGTGGCAGTTCTCATTCCTTTCCGTAATC |
| | GCCATGAACATCTTCCAATTTTTTTCTTACATCTGATTCCAATGCTCCAGAAGCAGCG |
| | GCTGGAATTTGCGTTTTATGTCATTGAACAGACTGGCACACAACCTTTTAACCGTGCG |
| | ATGCTTTTCAATGTGGGCTTCAAAGAGCCCATGAAACACAGTGTCTGGGACTGTGTAA |
| | TCTTCCACCATGTGGATCATCTACCTCAAAATGACCGGAACTATTACGGATGTGGAGA |
| | AATGCCACGTCATTTTGCTGCAAAGCTGGATAAATACATGTATATTCTTCCATATAAA |
| | GAATTTTTTGGTGGTGTAAGTGGGCTGACAGTGGAACAATTTAGAAAGATCAATGGTT |
| | TTCCTAATGCCTTCTGGGGATGGGGAGGAGAAGATGATGACCTTTGGAACAGAGTTCA |
| | CTATGCTGGATATAATGTAACCAGACCAGAGGGAGACTTAGGAAAATACAAGTCAATT |
| | CCTCATCACCATAGAGGTGAAGTCCAGTTTTTAGGACGGTATAAATTACTAAGGTATT |
| | CCAAGGAGCGTCAGTACATCGATGGACTGAACAATTTAATATATAGGCCAAAAATACT |
| | GGTTGATAGGTTGTATACAAACATATCTGTAAACCTCATGCCAGAGTTAGCTCCAATC |
| | GAAGACTATTAAAAGAAGTGGCTGTCGTGGCAAGGTAGACC |
| | ORF Start: ATG at 24 ORF Stop: TAA at 1170 |
| | SEQ ID NO: 286 382 aa MW at 44913.2kD |
| NOV20a, | MSVLRRMMRVSNRSLLAFIFFFSLSSSCLYFIYVAPGIANTYLFMVQARGIMLRENVK |
| CG152453-01 | |
| Protein Sequence | TIGHMIRLYTNKNSTLNGTDYPEGNNSSDYLVQTTTYLPENFTYSPYLPCPEKLPYMR |
| | GFLNVNVSEVSFDEIHQLFSKDLDIEPGGHWRPKDCKPRWKVAVLIPFRNRHEHLPIF |
| | FLHLIPMLQKQRLEFAFYVIEQTGTQPFNRAMLFNVGFKEAMKDSVWDCVIFHDVDHL |
| | PENDRNYYGCGEMPRHFAAKLDKYMYILPYKEFFGGVSGLTVEQFRKINGFPNAFWGW |
| | GGEDDDLWNRVHYAGYNVTRPEGDLGKYKSIPHHHRGEVQFLGRYKLLRYSKERQYID |
| | GLNNLIYRPKILVDRLYTNISVNLMPELAPIEDY |
| | SEQ ID NO: 287 1062 bp |
| NOV20b, | GATGTCTGTGCTCAGGCGGATGATGCGGGTTTCCAATCGCTCTCTCCTCGCCTTCATC |
| CG152453-03 | |
| DNA Sequence | TTCTTCTTCTCCCTCTCTTCGTCCTGTCTGTACTTCATCTATGTGGCCCCAGGCATCG |
| | ATTATCCCGAAGGCAATAATTCAAGTGATTATCTTGTTCAAACAACAACGTATCTCCC |
| | GGAAAACTTCACATACTCACCATACCTCCCCTGTCCAGAAAAGCTGCCTTATATGCGA |
| | GGATTCCTCAATGTCAATGTAAGCGAAGTCAGTTTTGATGAAATTCATCAACTCTTCT |
| | CCAAGGATTTAGATATTGAGCCAGGGGGTCATTGGAGGCCAAAAGACTGTAAACCCAG |
| | ATGGAAGGTGGCAGTTCTCATTCCTTTCCGTAATCGCCATGAACATCTTCCAATTTTT |
| | TTCTTACATCTGATTCCAATGCTCCAGAAGCAGCGGCTGGAATTTGCGTTTTATGTCA |
| | TTGAACAGACTGGCACACAACCTTTTAACCGTGCGATGCTTTTCAATGTGGGCTTCAA |
| | AGAGGCCATGAAAGACAGTGTCTGGGACTGTGTAATCTTCCACGATGTGGATCATCTA |
| | CCTGAAAATGACCGGAACTATTACGGATGTGGAGAAATGCCACGTCATTTTGCTGCAA |
| | AGCTGGATAAATACATGTATATTCTTCCATATAAAGAATTTTTTGGTGGTGTAAGTGG |
| | GCTGACAGTGGAACAATTTAGAAAGATCAATGGTTTTCCTAATGCCTTCTGGGGATCG |
| | GGAGGAGAAGATGATGACCTTTCGAACAGAGTTCACTATGCTGGATATAATGTAACCA |
| | GACCAGAGGGAGACTTAGGAAAATACAAGTCAATTCCTCATCACCATAGAGGTGAAGT |
| | CCAGTTTTTAGGACGGTATAAATTACTAAGGTATTCCAAGGAGCGTCAGTACATCGAT |
| | GGACTGAACAATTTAATATATAGGCCAAAAATACTGGTTGATAGGTTGTATACAAACA |
| | TATCTGTAAACCTCATGCCAGAGTTAGCTCCAATCGAAGACTATTAAAAGAAGTGGCT |
| | GTCGTGGCAAGGTAGACC |
| | ORF Start: ATG at 2 ORF Stop: TAA at 1031 |
| | SEQ ID NO: 288 343 aa MW at 40460.0kD |
| NOV20b, | MSVLRRMMRVSNRSLLAFIFFFSLSSSCLYFIYVAPGIDYPEGNNSSDYLVQTTTYLP |
| CG152453-03 | |
| Protein Sequence | ENFTYSPYLPCPEKLPYMRGFLNVNXTSEVSFDEIHQLFSKDLDIEPGGHWRPKDCKPR |
| | WKVAVLIPFRNRHEHLPIFFLHLIPMLQKQRLEFAFYVIEQTGTQPFNRAMLFNVGFK |
| | EAMKDSVWDCVIFHDVDHLPENDRNYYGCGEMPRHFAAKLDKYMYILPYKEFFGGVSC |
| | LTVEQFRKINGFPNAFWGWGGEDDDLWNRVHYAGYNVTRPEGDLGKYKSIPHHHRGEV |
| | QFLGRYKLLRYSKERQYIDGLNNLIYRPKILVDRLYTNISVNLMPELAPIEDY |
| | SEQ ID NO: 289 1100 bp |
| NOV20c, | ATGTCTGTGCTCAGGCGGATGATGCGGGTTTCCAATCGCTCTCTCCTCGCCTTCATCT |
| CG152453-02 | |
| DNA Sequence | TCTTCTTCTCCCTCTCTTCGTCCTGTCTGTACTTCATCTATGTGGCCCCAGGCATCGC |
| | CAACACACATCTCTTTATGGTACAAGCTCGAGGTATAATGTTGAGAGAAAATGTGAAA |
| | ACAATAGGTCATATGATCAGGCTGTACACAAATAAAAACAGTACGCTCAACGGTACAG |
| | ATTATCCCGAAGGCAATAATTCAAGTGATTATCTTGTTCAAACAACAACGTATCTCCC |
| | GGAAAACTTCACATACTCACCATACCTCCCCTGTCCAGAAAAGCTGCCTTATATGCGA |
| | GGATTCCTCAATGTCAATGTAAGCGAAGTCAGTTTTGATGAAATTCATCAACTCTTCT |
| | CCAAGGATTTAGATATTGAGCCAGGGGGTCATTGGAGGCCAAAAGACTGTAAACCCAG |
| | ATGGAAGAAGCAGCGGCTGGAATTTGCGTTTTATGTCATTGAACAGACTGGCACACAA |
| | CCTTTTAACCGTGCGATGCTTTTCAATGTGGGCTTCAAACAGGCCATGAAAGACAGTG |
| | TCTGGGACTGTGTAATCTTCCACGATGTGGATCATCTACCTGAAAATGACCGGAACTA |
| | TTACGGATGTGGAGAAATGCCACGTCATTTTGCTGCAAAGCTGGATAAATACATGTAT |
| | ATTCTTCCATATAAAGAATTTTTTGGTGGTGTAAGTGGGCTGACAGTGGAACAATTTA |
| | GAAAGATCAATGGTTTTCCTAATGCCTTCTGGGGATGGGCAGGAGAAGATGATGACCT |
| | TTGGAACAGAGTTCACTATGCTGGATATAATGTAACCAGACCAGAGGGAGACTTAGGA |
| | AAATACAAGTCAATTCCTCATCACCATAGAGGTGAAGTCCAGTTTTTAGGACGGTATA |
| | AATTACTAAGGTATTCCAAGGAGCGTCAGTACATCGATGGACTGAACAATTTAATATA |
| | TAGGCCAAAAATACTGGTTGATAGGTTGTATACAAACATATCTGTAAACCTCATGCCA |
| | GAGTTAGCTCCAATCGAAGACTATTAAAAGAAGTGGCTGTCGTGGCAAGGTAGACC |
| | ORF Start: ATG at 1 ORF Stop: TAA at 1069 |
| | SEQ ID NO: 290 356 aa MW at 41753.4kD |
| NOV20c, | MSVLRRMNRVSNRSLLAFIFFFSLSSSCLYFIYVAPGIANTHLFMVQARGIMLRENVK |
| CG152453-02 | |
| Protein Sequence | TIGHMIRLYTNKNSTLNGTDYPEGNNSSDYLVQTTTYLPENFTYSPYLPCPEKLPYMR |
| | GFLNVNVSEVSFDEIHQLFSKDLDIEPGGHWRPKDCKPRWKKQRLEFAFYVIEQTGTQ |
| | PFNRAMLFNVGFKEANKDSVWDCVIFHDVDHLPENDRNYYGCGEMPRHFAAKLDKYMY |
| | ILPYKEFFGGVSCLTVEQFRKINGFPNAFWGWGGEDDDLWNRVHYAGYNVTRPEGDLG |
| | KYKSIPHHHRGEVQFLGRYKLLRYSKERQYIDGLNNLIYRPKILVDRLYTNISVNLMP |
| | ELAPIEDY |

Sequence comparison of the above protein sequences yields the following sequence relationships shown in Table 20B.

TABLE 20B
Comparison of NOV20a against NOV20b and NOV20c.

| | | Identities/ |
|---|---|---|
| | | Similarities for |
| Protein | NOV20a Residues/ | the Matched |
| Sequence | Match Residues | Region |
| NOV20b | 1 . . . 382 | 343/382 (89%) |
| | 1 . . . 343 | 343/382 (89%) |
| NOV20c | 1 . . . 382 | 355/382 (92%) |
| | 1 . . . 356 | 356/382 (92%) |

Further analysis of the NOV20a protein yielded the following properties shown in Table 20C.

TABLE 20C
Protein Sequence Properties NOV20a

| | |
|---|---|
| PSort | 0.8541 probability located in lysosome (lumen); 0.7189 |
| analysis: | probability located in outside; 0.2757 probability |
| | located in microbody (peroxisome); 0.1000 probability |
| | located in endoplasmic reticulum (membrane) |
| SignalP | Cleavage site between residues 28 and 29 |
| analysis: | |

A search of the NOV20a protein against the Geneseq database, a proprietary database that contains sequences published in patents and patent publication, yielded several homologous proteins shown in Table 20D.

TABLE 20D
Geneseq Results for NOV20a

| | | | Identities/ | |
|---|---|---|---|---|
| | | | Similarities for | |
| Geneseq | Protein/Organism/Length | NOV20a Residues/ | the Matched | Expect |
| Identifier | [Patent #, Date] | Match Residues | Region | Value |
| AAW81569 | Human lactosyl ceramide synthase - | 1 . . . 382 | 382/382 (100%) | 0.0 |
| | Homo sapiens, 382 aa. | 1 . . . 382 | 382/382 (100%) | |
| | [JP10295371-A, 10 NOV. 1998] | | | |
| ABG23077 | Novel human diagnostic protein | 1 . . . 382 | 381/382 (99%) | 0.0 |
| | #23068 - Homo sapiens, 404 aa. | 23 . . . 404 | 382/382 (99%) | |
| | [WO200175067-A2, 11 OCT. 2001] | | | |
| AAW81567 | Rat lactosyl ceramide synthase - | 1 . . . 382 | 360/382 (94%) | 0.0 |
| | Rattus sp, 382 aa. [JP10295371-A, | 1 . . . 382 | 376/382 (98%) | |
| | 10 NOV. 1998] | | | |
| AAW81568 | Mouse lactosyl ceramide synthase - | 1 . . . 382 | 362/382 (94%) | 0.0 |
| | Mus sp, 382 aa. [JP10295371-A, | 1 . . . 382 | 374/382 (97%) | |
| | 10 NOV. 1998] | | | |
| AAB26791 | Human galactoside transferase | 1 . . . 382 | 342/382 (89%) | 0.0 |
| | I-type homologous protein - Homo | 1 . . . 343 | 343/382 (89%) | |
| | sapiens, 343 aa. [CN1257925-A, | | | |
| | 28 JUN. 2000] | | | |

In a BLAST search of public sequence datbases, the NOV20a protein was found to have homology to the proteins shown in the BLASTP data in Table 20E.

TABLE 20E
Public BLASTP Results for NOV20a

| | | | Identities/ | |
|---|---|---|---|---|
| Protein | | | Similarities for | |
| Accession | | NOV20a Residues/ | the Matched | Expect |
| Number | Protein/Organism/Length | Match Residues | Portion | Value |
| Q9UBX8 | Beta-1,4-galactosyltransferase 6 (EC | 1 . . . 382 | 382/382 (100%) | 0.0 |
| | 2.4.1.-) (Beta-1,4-GalTase 6) | 1 . . . 382 | 382/382 (100%) | |
| | (Beta4Gal-T6) (b4Gal-T6) (UDP- | | | |
| | galactose: beta-N-acetylglucosamine | | | |
| | beta- 1,4-galactosyltransferase 6) | | | |
| | (UDP-Gal: beta-GlcNAc beta-1,4- | | | |
| | galactosyltransferase 6) [Includes: | | | |
| | Lactosylceramide synthase (EC 2.4.1.-) | | | |
| | (LacCer synthase) (UDP- | | | |
| | Gal: glucosylceramide beta-1,4- | | | |
| | galactosyltransferase)] - Homo sapiens | | | |
| | (Human), 382 aa. | | | |
| O88419 | Beta-1,4-galactosyltransferase 6 (EC | 1 . . . 382 | 360/382 (94%) | 0.0 |
| | 2.4.1.-) (Beta-1,4-GalTase 6) | 1 . . . 382 | 376/382 (98%) | |
| | (Beta4Gal-T6) (b4Gal-T6) (UDP- | | | |
| | galactose: beta-N-acetylglucosamine | | | |
| | beta- 1,4-galactosyltransferase 6) | | | |
| | (UDP-Gal: beta-GlcNAc beta-1,4- | | | |
| | galactosyltransferase 6) [Includes: | | | |
| | Lactosylceramide synthase (EC 2.4.1.-) | | | |
| | (LacCer synthase) (UDP- | | | |
| | Gal: glucosylceramide beta-1,4- | | | |
| | galactosyltransferase)] - Rattus | | | |
| | norvegicus (Rat), 382 aa. | | | |
| Q9WVK5 | Beta-1,4-galactosyltransferase 6 | 1 . . . 382 | 362/382 (94%) | 0.0 |
| | (EC 2.4.1.-) (Beta-1,4-GalTase 6) | 1 . . . 382 | 374/382 (97%) | |
| | (Beta4Gal-T6) (b4Gal-T6) (UDP- | | | |
| | galactose: beta-N-acetylglucosamine | | | |
| | beta- 1,4-galactosyltransferase 6) | | | |
| | (UDP-Gal: beta-GlcNAc beta-1,4- | | | |
| | galactosyltransferase 6) [Includes: | | | |
| | Lactosylceramide synthase (EC 2.4.1.-) | | | |
| | (LacCer synthase) (UDP- | | | |
| | Gal: glucosylceramide beta-1,4- | | | |
| | galactosyltransferase)] - Mus musculus | | | |
| | (Mouse), 382 aa. | | | |
| Q8WZ95 | Beta-1,4-galactosyltransferase - Homo | 1 . . . 382 | 342/382 (89%) | 0.0 |
| | sapiens (Human), 343 aa. | 1 . . . 343 | 343/382 (89%) | |
| O43286 | Beta-1,4-galactosyltransferase 5 | 1 . . . 382 | 273/388 (70%) | e−169 |
| | (EC 2.4.1.-) (Beta-1,4-GalTase 5) | 1 . . . 388 | 321/388 (82%) | |
| | (Beta4Gal-T5) (b4Gal-T5) (UDP- | | | |
| | galactose: beta-N-acetylglucosamine | | | |
| | beta- 1,4-galactosyltransferase 5) | | | |
| | (UDP-Gal: beta-GlcNAc beta-1,4- | | | |
| | galactosyltransferase 5) (EC 2.4.1.-) | | | |
| | (Beta-1,4-GalT II) - Homo sapiens | | | |
| | (Human), 388 aa. | | | |

PFam analysis predicts that the NOV20a protein contains the domains shown in the Table 20F.

TABLE 20F
Domain Analysis of NOV20a

| | | Identities/ | |
|---|---|---|---|
| | | Similarities for | |
| Pfam | NOV20a | the Matched | Expect |
| Domain | Match Region | Region | Value |
| Galactosyl_T_2 | 108 . . . 375 | 157/329 (48%) | 3.2e−187 |
| | | 266/329 (81%) | |

### Example 21

The NOV21 clone was analyzed, and the nucleotide and encoded polypeptide sequences are shown in Table 21A.

TABLE 21A
NOV21 Sequence Analysis

| | |
|---|---|
| | SEQ ID NO: 291 1327 bp |
| NOV21a, | ATGGGCCGCTACTCTGGCAAGACGTGCCGGCTGCTCTTCATGCTGGTGCTCACCGTCG |
| CG152547-01 | |
| DNA Sequence | CCTTCTTCGTGGCGGAGCTGGTCTCCGGCTACCTGGGCAACTCCATCGCGCTGCTCTC |
| | CGACTCCTTCAACATGCTCTCCGACCTGATCTCGCTGTGCGTGGGCCTGAGCGCCGGC |
| | TACATCGCCCGGCGCCCCACCCGGGGCTTCAGCGCCACCTACGGCTACGCCCGCGCCG |
| | AGGTGGTGGGCGCGCTGAGCAACGCGGTCTTCCTCACCGCGCTCTGCTTCACCATCTT |
| | CGTGGAGGCCGTGCTGCGCCTGGCCCGGCCCGAGCGCATCGATGACCCCGAGCTGGTG |
| | CTCATCGTCGGCGTCCTGGGGCTGTTGGTCAACGTGGTGGGGCTGCTCATCTTCCATC |
| | ACCAATCCCTAATCTCAAGTAATCAGGGACACAAACACTGCGGAAGGCCGCAGGGTCC |
| | TCTGCCTAGGAAAACCAGAAACACCCAGAATGAGCCAGAAGACATGATGAAAAAAGAG |
| | AAAAAGTCTGAAGCTCTGAATATCAGAGGTGTACTTTTGCATGTGATGGGAGATGCCC |
| | TGGGGTCCGTGGTTGTGGTCATCACGGCCATCATATTCTATGTGCTTCCCCTGAAGAG |
| | TGAGGACCCGTGTAACTGGCAGTGTTACATTGACCCCAGCCTGACTGTCCTCATGGTC |
| | ATCATCATTTTGTCATCTGCCTTCCCGCTTATCAAGGAGACCGCTGCCATTCTGCTAC |
| | AGATGGTCCCAAAAGGAGTCAACATGGAAGAGCTGATGAGTAAACTCTCTGCTGTGCC |
| | TGGAATTAGCAGTGTACATGAAGTGCACATCTGGCAACTTGTAAGTGGAAAGATTATT |
| | GCCACCCTGCACATCAAGTATCCTAAGGACAGGGGATATCAAGATGCCAGCACAAAAA |
| | TTCGAGAAATCTTCCACCATGCGGGAATCCACAATGTGACCATCCAGTTTGAAAATGT |
| | GGACTTGAAGGAACCCCTGGAGCAGAAGGACTTACTGTTGCTCTGCAACTCACCCTGC |
| | ATCTCCAAGGGCTGTGCTAAGCAGCTGTGTTGTCCCCCCGGGGCACTGCCTCTGGCTC |
| | ACGTCAATGGCTGTGCTGAGCACAATGGTGGGCCCTCTCTAGACACATACGGAAGTGA |
| | TGGCCTCAGTAGAAGAGACGCAAGAGAAGTGGCTATTGAAGTGTCTTTGGATAGCTGT |
| | CTGAGTGACCACGGACAATGTCTTAACAAAACTCAGGAGGACCAATGTTATGTCAACA |
| | GAACGCATTTTTAATCTGGTACTCACATAATCAGACCATATAGACGAGAAG |
| | ORF Start: ATG at 1 ORF Stop: TAA at 1288 |
| | SEQ ID NO: 292 429 aa MW at 46990.2kD |
| NOV21a, | MGRYSGKTCRLLFMLVLTVAFFVAELVSGYLGNSIALLSDSFNMLSDLISLCVGLSAG |
| CG152547-01 | |
| Protein Sequence | YIARRPTRGFSATYGYARAEVVGALSNAVFLTALCFTIFVEAVLRLARPERIDDPELV |
| | LIVGVLGLLVNVVGLLIFHHQSLISSNQGHKHCGRPQGPLPRKTRNTQNEPEDMMKKE |
| | KKSEALNIRGVLLHVMGDALGSVVVVITAIIFYVLPLKSEDPCNWQCYIDPSLTVLMV |
| | IIILSSAFPLIKETAAILLQMXTPKGVNMEELMSKLSAVPGISSVHEVHIWELVSGKII |
| | ATLHIKYPKDRGYQDASTKIREIFHAGIHNVTIQFENVDLKEPLEQKDLLLLCNSPC |
| | ISKGCAKQLCCPPGALPLAHVNGCAEHNGGPSLDTYGSDGLSRRDAREVAIEVSLDSC |
| | LSDHCQCLNKTQEDQCYVNRTHF |

Further analysis of the NOV21a protein yielded the following properties shown in Table 21B.

TABLE 21B
Protein Sequence Properties NOV21a

| | |
|---|---|
| PSort | 0.6400 probability located in plasma membrane; |
| analysis: | 0.4600 probability located in Golgi body; |
| | 0.3700 probability located in endoplasmic |
| | reticulum (membrane); 0.1000 probability |
| | located in endoplasmic reticulum (lumen) |
| SignalP | Cleavage site between residues 30 and 31 |
| analysis: | |

A search of the NOV21a protein against the Geneseq database, a proprietary database that contains sequences published in patents and patent publication, yielded several homologous proteins shown in Table 21C.

TABLE 21C
Geneseq Results for NOV21a

| | | | Identities/ | |
|---|---|---|---|---|
| | | | Similarities for | |
| Geneseq | Protein/Organism/Length | NOV21a Residues/ | the Matched | Expect |
| Identifier | [Patent #, Date] | Match Residues | Region | Value |
| ABP51303 | Human MDDT SEQ ID NO 325 - | 1 . . . 429 | 410/485 (84%) | 0.0 |
| | Homo sapiens, 520 aa. | 36 . . . 520 | 413/485 (84%) | |
| | [WO200240715-A2, 23 MAY 2002] | | | |
| AAU99906 | Human 83378 metal transporter | 1 . . . 429 | 408/485 (84%) | 0.0 |
| | protein - Homo sapiens, 485 aa. | 1 . . . 485 | 411/485 (84%) | |
| | [WO200240656-A2, 23 MAY 2002] | | | |
| AAM52621 | Human zinc ion transport protein | 190 . . . 429 | 238/240 (99%) | e−138 |
| | 26 - Homo sapiens, 240 aa. | 1 . . . 240 | 238/240 (99%) | |
| | [WO200181539-A2, 01 NOV. 2001] | | | |
| AAG66785 | Zinc transporter homologue ZnT-1- | 231 . . . 429 | 197/199 (98%) | e−112 |
| | 22 - Homo sapiens, 199 aa. | 1 . . . 199 | 197/199 (98%) | |
| | [WO200171000-A1, 27 SEP. 2001] | | | |
| AAU69449 | Human purified secretory | 1 . . . 290 | 240/346 (69%) | e−111 |
| | polypeptide #18 - Homo sapiens, | 36 . . . 349 | 243/346 (69%) | |
| | 349 aa. [WO200162918-A2, | | | |
| | 30 AUG. 2001] | | | |

In a BLAST search of public sequence datbases, the NOV21a protein was found to have homology to the proteins shown in the BLASTP data in Table 21D.

TABLE 21D
Public BLASTP Results for NOV21a

| | | | Identities/ | |
|---|---|---|---|---|
| Protein | | | Similarities for | |
| Accession | | NOV21a Residues/ | the Matched | Expect |
| Number | Protein/Organism/Length | Match Residues | Portion | Value |
| Q9NPW0 | Hypothetical 26.3 kDa protein - | 190 . . . 429 | 239/240 (99%) | e−138 |
| | Homo sapiens (Human), 240 aa. | 1 . . . 240 | 239/240 (99%) | |
| Q9Y6M5 | Zinc transporter 1 (ZnT-1) - Homo | 1 . . . 398 | 181/493 (36%) | 2e−72 |
| | sapiens (Human), 507 aa. | 1 . . . 485 | 249/493 (49%) | |
| Q9VZR4 | CG17723 protein (LD22804P) - | 1 . . . 359 | 148/390 (37%) | 5e−68 |
| | Drosophila melanogaster (Fruit | 1 . . . 378 | 228/390 (57%) | |
| | fly), 449 aa. | | | |
| Q06808 | Oxidative stress resistance - | 5 . . . 351 | 143/402 (35%) | 6e−61 |
| | Saccharomyces cerevisiae (Baker's | 3 . . . 398 | 222/402 (54%) | |
| | yeast), 429 aa. | | | |
| P20107 | Zinc/cadmium resistance protein - | 5 . . . 351 | 143/402 (35%) | 6e−61 |
| | Saccharomyces cerevisiae (Baker's | 3 . . . 398 | 222/402 (54%) | |
| | yeast), 442 aa. | | | |

PFam analysis predicts that the NOV21a protein contains the domains shown in the Table 21E.

TABLE 21E
Domain Analysis of NOV21a

| | | Identities/ | |
|---|---|---|---|
| | | Similarities for | |
| Pfam | NOV21a | the Matched | Expect |
| Domain | Match Region | Region | Value |
| Cation_efflux | 11 . . . 333 | 101/358 (28%) | 2.2e−68 |
| | | 259/358 (72%) | |

### Example 22

The NOV22 clone was analyzed, and the nucleotide and encoded polypeptide sequences are shown in Table 22A.

TABLE 22A
NOV22 Sequence Analysis

| | |
|---|---|
| | SEQ ID NO: 293 1047 bp |
| NOV22a, | AGGCTGGGCACGAGGACCATGCTGGGCCGGAGCCTCCGAGAAGTTTCTGCGGCACTGA |
| CG152646-01 | |
| DNA Sequence | AACAAGGCCAAATTACACCAACAGAGCTCTGTCAAAAATGTCTCTCTCTTATCAAGAA |
| | GACCAAGTTTCTAAATGCCTACATTACTGTGTCAGAAGAGGTGGCCTTAAAACAAGCT |
| | GAAGAATCAGAAAAGAGATATAAGAATGGACAGTCACTTGGGGATTTAGATGGAATTC |
| | CTATTGCAGTAAAAGACAATTTCAGCACTTCTGGCATTGAGACAACATGTGCATCAAA |
| | TATGCTGAAAGGTTATATACCACCTTATAATGCTACAGTAGTTCAGAAGTTGTTGGAT |
| | CAGGGAGCTCTACTAATGGGAAAAACAAATTTAGATGAGTTTGCTATGGGATCTGGGA |
| | GCACAGATGGTGTATTTGGACCAGTTAPAAACCCCTGGAGTTATTCAAAACAATATGG |
| | TCACAGATGTGACATTGATTTGTCCACTGAAGCCATGTATGCTGCAACCAGACGAGAA |
| | GGGTTTAATGATGTGGTGAGAGGAAGAATTCTCTCAGGAAACTTTTTCTTATTAAAAG |
| | AAAACTATGAAAATTATTTTGTCAAAGCACAGAAAGTGAGACGCCTCATTGCTAATGA |
| | CTTTGTAAATGCTTTTAACTCTGGAGTAGATGTCTTGCTAACTCCCACCACCTTGAGT |
| | GAGGCAGTACCATACTTGGAGTTCATCAAAGAGGACAACAGAACCCGAAGTGCCCAGG |
| | ATGATATTTTTACACAAGCTGTAAATATGGCAGGATTGCCAGCAGTGAGTATCCCTGT |
| | TGCACTCTCAAACCAGGGGTTGCCAATAGGACTGCAGTTTATTGGACGTGCGTTTTGT |
| | GACCAGCAGCTTCTTACAGTAGCCAAATGGTTTGAAAAACAAGTACAGTTTCCTGTTA |
| | TTCAACTTCAAGAACTCATGGATGATTGTTCAGCAGTCCTTGAAAATGAAAAGTTAGC |
| | CTCTGTCTCTCTAAAACAGTAAACATATCTTACAAATTAAAATGACTTTTAGGCTGGG |
| | TGC |
| | ORF Start: ATG at 19 ORF Stop: TAA at 1006 |
| | SEQ ID NO: 294 329 aa MW at 36411.3kD |
| NOV22a, | MLGRSLREVSAALKQGQITPTELCQKCLSLIKKTKFLNAYITVSEEVALKQAEESEKR |
| CG152646-01 | |
| Protein Sequence | YKNGQSLGDLDGIPIAVKDNFSTSGTETTCASNMLKGYIPPYNATVVQKLLDQGALLM |
| | GKTNLDEFAMGSGSTDGVFGPVKNPWSYSKQYGHRCDIDLSTEANYAATRREGFNDVV |
| | RGRILSGNFFLLKENYENYFVKAQKVRRLIANDFVNAFNSGVDVLLTPTTLSEAVPYL |
| | EFIKEDNRTRSAQDDIFTQAVNMAGLPAVSIPVALSNQGLPIGLQFIGRAFCDQQLLT |
| | VAKWFEKQVQFPVIQLQELMDDCSAVLENEKLASVSLKQ |

Further analysis of the NOV22a protein yielded the following properties shown in Table 22B.

TABLE 22B
Protein Sequence Properties NOV22a

| | |
|---|---|
| PSort | 0.6500 probability located in cytoplasm; |
| analysis: | 0.1000 probability located in mitochondrial |
| | matrix space; 0.1000 probability located in |
| | lysosome (lumen); 0.0000 probability located |
| | in endoplasmic reticulum (membrane) |
| SignalP | No Known Signal Sequence Predicted |
| analysis: | |

A search of the NOV22a protein against the Geneseq database, a proprietary database that contains sequences published in patents and patent publication, yielded several homologous proteins shown in Table 22C.

TABLE 22C
Geneseq Results for NOV22a

| | | | Identities/ | |
|---|---|---|---|---|
| | | | Similarities for | |
| Geneseq | Protein/Organism/Length | NOV22a Residues/ | the Matched | Expect |
| Identifier | [Patent #, Date] | Match Residues | Region | Value |
| ABP41274 | Human ovarian antigen HOSED43, | 147 . . . 329 | 182/183 (99%) | e−100 |
| | SEQ ID NO: 2406 - Homo sapiens, | 81 . . . 263 | 183/183 (99%) | |
| | 263 aa. [WO200200677-A1, | | | |
| | 03 JAN. 2002] | | | |
| ABB05695 | Human nucleic acid management | 147 . . . 329 | 182/183 (99%) | e−100 |
| | protein clone fbr2_78c12 - Homo | 346 . . . 528 | 183/183 (99%) | |
| | sapiens, 528 aa. [WO200198454- | | | |
| | A2, 27 DEC. 2001] | | | |
| AAE18112 | Human glutamyl-tRNA (Gln) | 147 . . . 329 | 182/183 (99%) | e−100 |
| | amidotransferase−like enzyme - | 346 . . . 528 | 183/183 (99%) | |
| | Homo sapiens, 528 aa. | | | |
| | [WO200200703-A2, 03 JAN. 2002] | | | |
| AAU19422 | Human diagnostic and therapeutic | 147 . . . 329 | 182/183 (99%) | e−100 |
| | polypeptide (DITHP) #8 - Homo | 367 . . . 549 | 183/183 (99%) | |
| | sapiens, 549 aa. [WO200162927- | | | |
| | A2, 30 AUG. 2001] | | | |
| AAB94654 | Human protein sequence SEQ ID | 147 . . . 329 | 182/183 (99%) | e−100 |
| | NO: 15566 - Homo sapiens, 528 aa. | 346 . . . 528 | 183/183 (99%) | |
| | [EP1074617-A2, 07 FEB. 2001] | | | |

In a BLAST search of public sequence datbases, the NOV22a protein was found to have homology to the proteins shown in the BLASTP data in Table 22D.

TABLE 22D
Public BLASTP Results for NOV22a

| | | | Identities/ | |
|---|---|---|---|---|
| Protein | | | Similarities for | |
| Accession | | NOV22a Residues/ | the Matched | Expect |
| Number | Protein/Organism/Length | Match Residues | Portion | Value |
| Q9NV19 | Hypothetical 57.5 kDa protein | 147 . . . 329 | 182/183 (99%) | e−100 |
| | (Similar to hypothetical protein | 346 . . . 528 | 183/183 (99%) | |
| | FLJ10989) - Homo sapiens | | | |
| | (Human), 528 aa. | | | |
| Q9H0R6 | Hypothetical 57.5 kDa protein - | 147 . . . 329 | 182/183 (99%) | e−100 |
| | Homo sapiens (Human), 528 aa. | 346 . . . 528 | 183/183 (99%) | |
| Q9CZN8 | 2700038P16Rik protein - Mus | 147 . . . 329 | 163/183 (89%) | 6e−88 |
| | musculus (Mouse), 525 aa. | 342 . . . 524 | 169/183 (92%) | |
| Q9HA60 | CDNA FLJ12189 fis, clone | 1 . . . 148 | 148/148 (100%) | 4e−80 |
| | MAMMA1000841, moderately | 1 . . . 148 | 148/148 (100%) | |
| | similar to putative amidase (EC | | | |
| | 3.5.1.4) - Homo sapiens (Human), | | | |
| | 303 aa. | | | |
| Q9VE09 | GATA protein - Drosophila | 147 . . . 305 | 89/164 (54%) | 6e−43 |
| | melanogaster (Fruit fly), 508 aa. | 336 . . . 499 | 114/164 (69%) | |

PFam analysis predicts that the NOV22a protein contains the domains shown in the Table 22E.

TABLE 22E
Domain Analysis of NOV22a

| | | Identities/ | |
|---|---|---|---|
| | NOV22a | Similarities for the | |
| Pfam Domain | Match Region | Matched Region | Expect Value |
| Amidase | 22 . . . 142 | 58/126 (46%) | 1.5e-41 |
| | | 98/126 (78%) | |
| Amidase | 148 . . . 289 | 62/170 (36%) | 7.6e-35 |
| | | 114/170 (67%) | |

### Example 23

The NOV23 clone was analyzed, and the nucleotide and encoded polypeptide sequences are shown in Table 23A.

TABLE 23A
NOV23 Sequence Analysis

| | |
|---|---|
| | SEQ ID NO: 295 1935 bp |
| NOV23a, | AGAGGCTCAAGAGGGGCAGCCCCCGCATAGAGGAGATGCGAGCTCTGCGCTCTGCCAG |
| CG152959-01 | |
| DNA Sequence | GGCCCCGAGCCCGTCAGAGGCCGCCCCGCGCCGCCCGGAAGCCACCGCGGCCCCCCTC |
| | ACTCCTAGAGGAAGGGAGCACCGCGAGGCTCACGGCAGGGCCCTGGCGCCGGGCAGGG |
| | CGAGCCTCGGAAGCCGCCTGGAGGACGTCCTGTGGCTGCAGGAGGTCTCCAACCTGTC |
| | AGAGTGGCTGAGTCCCAGCCCTGGGCCCTGAGCCGGGTCCCCTTCCGCAAGCGCCCAC |
| | CGATCCGGAGGCTGCGGGCAGCCGTTATCCCGTGGTTTAATAAAGCTGCCGCGCGCTC |
| | ACCAAGTCCTCTTCCGCGTCTGCTTCCGCGTCGGGCCCGGGCGGGGCGGGGCGGGGCG |
| | TGGAGCCGCGCCGCGGCCTGACGTCACCCACACCTCCCTGGGACTGCGTCACTGGTCC |
| | GCGCCGCGGGTCAGGGCGCAATGGCGGCGCTGGGCGGGGATGGGCTGCGACTGCTGTC |
| | GGTGTCGCGGCCGGAGCGGCCGCCCGAGTCGGCGGCGCTGGGCGGCCTGGGCCCCGGG |
| | CTGTGCTGCTGGGTGTCAGTGTTCTCCTGCCTCAGCCTCGCCTGCTCCTACATGGGCA |
| | GCCTCTACGTCTGGAAGAGCGAACTGCCCAGGGACCATCCCGCGGTCATCAAGCGACG |
| | CTTCACCAGCGTCCTGGTGGTGTCCAGTCTCTCACCCCTGTGCGTGCTGCTCTGGAGG |
| | GAACTCACAGGCATCCAGGCACATCCCTGCTCACCCTGATGGGCTTCAGGCTGGAGGG |
| | CATTTTCCCAGCGGCGCTGCTGCCCCTGTTGCTGACCATGATTCTTTTCCTGGGCCCA |
| | CTGATGCAGCTCTCTATGGATTGCCCTTGTGACCTGGCAGATGGGCTGAAGGTTGTCC |
| | TGGCCCCCCGCTCCTGGGCCCGCTGCCTCACAGACATGCGTTGGCTGCGGAACCAAGT |
| | GATCGCCCCGCTGACAGAGGAGCTGGTGTTCCGGGCCTGTATGCTGCCCATGTTAGCA |
| | CCGTGCATGGGCCTGGGCCCTGCTGTGTTCACCTGCCCGCTCTTTTTTGGAGTTGCCC |
| | ATTTTCACCATATTATTGAGCAGCTGCGTTTCCGCCAGAGCAGCGTGGGGAACATCTT |
| | CTTGTCTGCTGCGTTCCAGTTCCCCTACACAGCTGTCTTCGGTGCCTACACTGCTTTC |
| | CTCTTCATCCGCACAGGACACCTGATTGGGCCGGTTCTCTGCCATTCCTTCTGCAATT |
| | ACATGGGTTTCCCAGCTGTTTGCGCGGCCTTGGAGCACCCACAGAGGCGGCCCCTGCT |
| | GGCAGGCTATGCCCTGGGTGTGGGACTCTTCCTGCTTCTGCTCCAGCCCCTCACGGAC |
| | CCCAAGCTCTACGGCAGCCTTCCCCTTTGTGTGCTTTTGGAGCGGGCAGGGGACTCAG |
| | AGGCTCCCCTGTGCTCCTGACCTATGCTCCTGGATACGCTATGAACTCTCACCGGCTC |
| | CCCAGCCCTCCCCACCAAGGGGTACTGCAGGGGAAGGGCTGGCTGGGGTCCCCGAGAT |
| | CTCAGGAATTTTTGTAGGGGATTGAAGCCAGAGCTAGTTGCGTCCCAGGGACCAAGAG |
| | AAAGAAGCAGATATCCAAAGGGTGCAGCCCCTTTTGAAAGGGGTGTTTACGAGCAGCT |
| | GTGAGTGAGGGGACAAGGGGCACGTCCCAGGAGCCACACACTCCCTTCCTCACTTTGG |
| | ACTGCTGCTTCTCTTAGCTCCTCTGCCTCTGAAAAGCTGCTCGGGGTTTTTTATTTAT |
| | AAAACCTCTCCCCACCCCCCACCCCCCAACTTCCTGGGTTTTCTCATTGTCTTTTTGC |
| | ATCAGTACTTTGTATTGGGATATTAAAGAGATTTAACTTGGGTAAAAAAAAAAAAAAA |
| | AAAAAAAAAAAAAAAAAAAAA |
| | ORF Start: ATG at 475 ORF Stop: TGA at 791 |
| | SEQ ID NO: 296 102 aa MW at 10925.7kD |
| NOV 23a, | MAALGGDGLRLLSVSRPERPPESAALGGLGPGLCCWVSVFSCLSLACSYMGSLYVWKS |
| CG152959-01 | |
| Protein Sequence | ELPRDHPAVIKRRFTSVLVVSSLSPLCVLLWRELTGIQAHPCSP |
| | SEQ ID NO: 297 1472 bp |
| NOV23b, | GTCACTGGTGCGCGCCGCGGGTCAGGGCGCAATGGCGGCGCTGGGCGGGGATGGGCTG |
| CG152959-02 | |
| DNA Sequence | CGACTGCTGTCGGTGTCGCGGCCGGAGCGGCCGCCCCAGTCGGCGGCGCTGGGCGGCC |
| | TGGGCCCCGGGCTGTGCTGCTGGGTGTCAGTGTTCTCCTGCCTCAGCCTCGCCTGCTC |
| | CTACGTGGGCAGCCTCTACGTCTGGAAGAGCGAACTGCCCAGGGACCATCCCGCGGTC |
| | ATCAAGCGACGCTTCACCAGCGTCCTGGTGGTGTCCAGTCTCTCACCCCTGTGCGTGC |
| | TGCTCTGGAGGGAACTCACAGGCATCCAGCCAGGCACATCCCTGCTCACCCTGATGGG |
| | CTTCAGGCTGGAGGGCATTTTCCCAGCGGCGCTGCTGCCCCTGTTGCTGACCATGATT |
| | CTTTTCCTGGGCCCACTGATGCAGCTCTCTATGGATTGCCCTTGTGACCTGGCAGATG |
| | GGCTGAAGGTTGTCCTGGCCCCCCGCTCCTGGGCCCGCTGCCTCACAGACATGCGTTG |
| | GCTGCGGAACCAAGTGATCGCCCCGCTGACAGAGGAGCTGGTGTTCCGGGCCTGTATG |
| | CTGCCCATGTTAGCACCGTGCATGGGCCTGGGCCCTGCTGTGTTCACCTGCCCGCTCT |
| | TTTTTGGAGTTGCCCATTTTCACCATATTATTGAGCAGCTGCGTTTCCGCCAGAGCAG |
| | CGTGGGGAACATCTTCTTGTCTGCTGCGTTCCAGTTCTCCTACACAGCTGTCTTCGGT |
| | CCCTACACTGCTTTCCTCTTCATCCGCACAGGACACCTGATTGGGCCGGTTCTCTGCC |
| | ATTCCTTCTGCAATTACATGGGTTTCCCAGCTGTTTGCGCGGCCTTGGAGCACCCACA |
| | GAGGCGGCCCCTGCTGGCAGGCTATGCCCTGGGTGTGGGACTCTTCCTGCTTCTGCTC |
| | CAGCCCCTCACGGACCCCAAGCTCTACGGCAGCCTTCCCCTTTGTGTGCTTTTGGAGC |
| | GGGCAGGGGACTCAGAGGCTCCCCTGTGCTCCTGACCTATGCTCCTGGATACGCTATG |
| | AACTCTCACCGGCTCCCCAGCCCTCCCCACCAAGGGGTACTGCAGGGGAAGGGCTGGC |
| | TGGGGTCCCCGAGATCTCAGGAATTTTTGTAGGGGATTGAAGCCAGAGCTAGTTGCGT |
| | CCCAGGGACCAAGAGAAAGAAGCAGATATCCAAAGGGTGCAGCCCCTTTTGAAAGGGG |
| | TGTTTACGAGCAGCTGTGAGTGAGGGGACAAGGGGCAGGTCCCAGGAGCCACACACTC |
| | CCTTCCTCACTTTGGACTGCTGCTTCTCTTAGCTCCTCTGCCTCTGAAAAGCTGCTCG |
| | GGGTTTTTTATTTATAAAACCTCTCCCCACCCCCCACCCCCCAAACTTCCTGGGTTTT |
| | CTCATTGTCTTTTTGCATCAGTACTTTGTATTGGGATATTAAAGAGATTTAACTTGGG |
| | TAAAAAAAAAAAAAAAAAAAA |
| | ORF Start: ATG at 32 ORE Stop: TGA at 1019 |
| | SEQ ID NO: 298 329 aa MW at 35832.2kD |
| NOV23b, | MAALGGDGLRLLSVSRPERPPESAALGGLGPGLCCWVSVFSCLSLACSYVGSLYVWKS |
| CG152959-02 | |
| Protein Sequence | ELPRDHPAVIKRRFTSVLVVSSLSPLCVLLWRELTGIQPGTSLLTLMGFRLEGIFPAA |
| | LLPLLLTMILFLGPLMQLSMDCPCDLADGLKVVLAPRSWARCLTDMRWLRNQVIAPLT |
| | EELVFRACMLPMLAPCMGLGPAVFTCPLFFGVAHFHHIIEQLRFRQSSVGNIFLSAAF |
| | QFSYTAVFGAYTAFLFIRTGHLIGPVLCHSFCNYMGFPAVCAALEHPQRRPLLAGYAL |
| | GVGLFLLLLQPLTDPKLYGSLPLCVLLERAGDSEAPLCS |

Sequence comparison of the above protein sequences yields the following sequence relationships shown in Table 23B.

TABLE 23B
Comparison of NOV23a against NOV23b.

| | NOV23a Residues/ | Identities/Similarities |
|---|---|---|
| Protein Sequence | Match Residues | for the Matched Region |
| NOV23b | 1 . . . 96 | 95/96 (98%) |
| | 1 . . . 96 | 96/96 (99%) |

Further analysis of the NOV23a protein yielded the following properties shown in Table 23C.

TABLE 23C
Protein Sequence Properties NOV23a

| | |
|---|---|
| PSort | 0.7000 probability located in plasma membrane; 0.2000 |
| analysis: | probability located in endoplasmic reticulum (membrane); |
| | 0.1000 probability located in mitochondrial inner membrane; |
| | 0.0000 probability located in endoplasmic reticulum (lumen) |
| SignalP | Cleavage site between residues 49 and 50 |
| analysis: | |

A search of the NOV23a protein against the Geneseq database, a proprietary database that contains sequences published in patents and patent publication, yielded several homologous proteins shown in Table 23D.

TABLE 23D
Geneseq Results for NOV23a

| | | | Identities/ | |
|---|---|---|---|---|
| | | NOV23a | Similarities | |
| | Protein/Organism/ | Residues/ | for the | |
| Geneseq | Length [Patent | Match | Matched | Expect |
| Identifier | #, Date] | Residues | Region | Value |
| AAY55809 | Human RCE1 | 1 . . . 96 | 95/96 (98%) | 5e-51 |
| | (farnesyl-directed | 1 . . . 96 | 96/96 (99%) | |
| | endopeptidase) se- | | | |
| | quence - Homo | | | |
| | sapiens, 329 aa. | | | |
| | [WO9961628-A2, | | | |
| | 02-DEC-1999] | | | |
| AAW89181 | Human RCE1 | 1 . . . 96 | 95/96 (98%) | 5e-51 |
| | (hRCE1) polypep- | 1 . . . 96 | 96/96 (99%) | |
| | tide - Homo sapiens, | | | |
| | 329 aa. [EP887415- | | | |
| | A2, 30-DEC-1998] | | | |
| AAW98105 | Guman ras carboxy- | 1 . . . 96 | 95/96 (98%) | 5e-51 |
| | terminal processing | 10 . . . 105 | 96/96 (99%) | |
| | protein - Homo | | | |
| | sapiens, 338 aa. | | | |
| | [WO9914343-A1, | | | |
| | 25-MAR-1999] | | | |
| AAY26897 | Human farnesyla- | 1 . . . 96 | 95/96 (98%) | 5e-51 |
| | ted--protein convert- | 1 . . . 96 | 96/96 (99%) | |
| | ing enzyme 2 pro- | | | |
| | tein - Homo sapiens, | | | |
| | 329 aa. | | | |
| | [WO9935275-A1, | | | |
| | 15-JUL-1999] | | | |
| AAU03600 | Human ras convert- | 1 . . . 96 | 94/96 (97%) | 1e-50 |
| | ing endoprotease | 1 . . . 96 | 96/96 (99%) | |
| | (RCE) - Homo sa- | | | |
| | piens, 329 aa. | | | |
| | [US6261793-B1, | | | |
| | 17-JUL-2001] | | | |

In a BLAST search of public sequence datbases, the NOV23a protein was found to have homology to the proteins shown in the BLASTP data in Table 23E.

TABLE 23E
Public BLASTP Results for NOV23a

| | | | Identities/ | |
|---|---|---|---|---|
| | | NOV23a | Similarities | |
| Protein | | Residues/ | for the | |
| Accession | Protein/Organism/ | Match | Matched | Expect |
| Number | Length | Residues | Portion | Value |
| Q9Y256 | CAAX prenyl pro- | 1 . . . 96 | 95/96 (98%) | 1e-50 |
| | tease 2 (EC | 1 . . . 96 | 96/96 (99%) | |
| | 3.4.22.-) (Prenyl | | | |
| | protein-specific en- | | | |
| | doprotease 2) | | | |
| | (Farnesylated-pro- | | | |
| | teins converting | | | |
| | enzyme 2) (FACE- | | | |
| | 2) (hRCE1) - Homo | | | |
| | sapiens (Human), | | | |
| | 329 aa. | | | |
| P57791 | CAAX prenyl pro- | 1 . . . 96 | 89/96 (92%) | 8e-46 |
| | tease 2 (EC | 1 . . . 96 | 90/96 (93%) | |
| | 3.4.22.-) (Prenyl | | | |
| | protein-specific en- | | | |
| | doprotease 2) | | | |
| | (Farnesylated-pro- | | | |
| | teins converting | | | |
| | enzyme 2) (FACE- | | | |
| | 2) - Mus musculus | | | |
| | (Mouse), 329 aa. | | | |
| Q9CSF8 | Ras and a-factor- | 28 . . . 96 | 63/69 (91%) | 2e-31 |
| | converting enzyme | 13 . . . 81 | 65/69 (93%) | |
| | 1 homolog (S. | | | |
| | cerevisiae) - Mus | | | |
| | musculus (Mouse), | | | |
| | 314 aa (fragment). | | | |
| Q8SZZ3 | LD46418p - | 38 . . . 86 | 24/49 (48%) | 2e-06 |
| | Drosophila | 30 . . . 78 | 31/49 (62%) | |
| | melanogaster (Fruit | | | |
| | fly), 302 aa. | | | |
| Q9U1H8 | CAAX prenyl pro- | 38 . . . 86 | 24/49 (48%) | 2e-06 |
| | tease 2 (EC | 18 . . . 66 | 31/49 (62%) | |
| | 3.4.22.-) (Prenyl | | | |
| | protein-specific en- | | | |
| | doprotease 2) | | | |
| | (Farnesylated-pro- | | | |
| | teins converting | | | |
| | enzyme 2) (FACE- | | | |
| | 2) (Severas pro- | | | |
| | tein) - | | | |
| | Drosophila | | | |
| | melanogaster (Fruit | | | |
| | fly), 290 aa. | | | |

PFam analysis predicts that the NOV23a protein contains the domains shown in the Table 23F.

TABLE 23F
Domain Analysis of NOV23a

| | | | |
|---|---|---|---|
| | | Identities/ | |
| Pfam | NOV23a | Similarities | |
| Domain | Match Region | for the Matched Region | Expect Value |

No Significant Matches Found

### Example 24

The NOV24 clone was analyzed, and the nucleotide and encoded polypeptide sequences are shown in Table 24A.

TABLE 24A
NOV24 Sequence Analysis

| | |
|---|---|
| | SEQ ID NO: 299 1710 bp |
| NOV24a, | ATGGCTGGACCAGGCAAAGAGGGTGTGGTGTGGTGGGAAGAAAAATCGATGGGACAAC |
| CG153033-01 | |
| DNA Sequence | TGAGGGAAGAAGATAACATTGAGCTGAATGAAGAAGGAAGGCCGGTGCAGACGTCCAG |
| | GCCAAGCCCCCCACTCTGCGACTGCCACTGCTGCGGCCTCCCCAAGCGTTACATCATT |
| | GCTATCATGAGTGGGCTGGGATTCTGCATTTCCTTTGGGATCCGGTGCAATCTTGGAG |
| | TTGCCATTGTGGAAATGGTCAACAATAGCACCGTATATGTTGATGGAAAACAGACAGC |
| | ACAGTTTAACTGGGATCCAGAAACAGTGGGCCTTATCCATGGATCTTTTTTCTGGGGC |
| | TATATTATGACACAAATTCCAGGTGGTTTCATTTCAAACAAGTTTGCTGCTAACAGGG |
| | TCTTTGGAGCTGCCATCTTCTTAACATCGACTCTGAACATGTTTATTCCCTCTGCAGC |
| | CAGAGTGCATTACGGATGCGTCATGTGTGTCAGAATTCTGCAAGGTTTAGTGGGTGTG |
| | ACCTACCCAGCCTGCCATGGGATGTGGAGTAAGTGGGCACCACCTTTGGAGAGAAGCC |
| | GACTGGCCACAACCTCTTTTTGTGGTTCCTATGCAGGGGCAGTGGTTGCCATGCCCCT |
| | GGCTGGGGTGTTGGTGCAGTACATTGGATGGTCCTCTGTCTTTTATATTTATGGTATG |
| | TTTGGGATTATTTGGTACATGTTTTGGCTGTTGCAGGCCTATGAGTGCCCAGCAGCTC |
| | ATCCAACAATATCCAATGAGGAGAAGACCTATATAGAGACAAGCATAGGAGAGGGGGC |
| | CAACGTGGTTAGTCTAAGTGTAAAATTTAGTACCCCATGGAAAAGATTTTTCACATCT |
| | TTGCCGGTTTATGCAATCATTGTGGCAAATTTTTGCAGAAGCTGGACCTTTTATTTGC |
| | TCCTCATAAGTCAGCCTGCTTATTTTGAAGAGGTCTTTGGATTTGCAATAAGTAAGGT |
| | AGGTCTCTTGTCAGCAGTCCCACACATGGTTATGACAATCGTTGTACCTATTGGAGGA |
| | CAATTGGCTGATTATTTAAGAAGCAGACAAATTTTAACCACAACTGCTGTCAGAAAAA |
| | TCATGAACTGTGGAGGTTTTGGCATGGAGGCAACCTTACTCCTGGTGGTTGGCTTTTC |
| | GCATACCAAAGGGGTGGCTATCTCCTTTCTGGTACTTGCTGTAGGATTTAGTGCCTTC |
| | GCTATTTCAGGTTTTAATGTCAACCACCTGGACATTGCCCCACGCTATGCCAGCATTC |
| | TCATGGGGATCTCAAACGGACTGGGAACCCTCTCTGGAATGGTCTGTCCCCTCATTGT |
| | CGGTGCAATGACCAGGCACAAGACCCGTGAAGAATGGCAGAATGTGTTCCTCATAGCT |
| | GCCCTGGTGCATTACAGTGGTGTGATCTTCTATGGGGTCTTTGCTTCTGGGGAGAAAC |
| | AGGAGTGGGCTGACCCAGAGAATCTCTCTGAGGAGAAATGTGGAATCATTGACCAGGA |
| | CGAATTAGCTGAGGAGATAGAACTCAACCATGAGAGTTTTGCGAGTCCCAAAAAGAAG |
| | ATGTCTTATGGAGCCACCTCCCAGAATTGTGAAGTCCAGAAGAAGGAATGGAAAGGAC |
| | AGAGAGGAGCGACCCTTGATGAGGAAGAGCTGACATCCTACCAGAATGAAGAGAGAAA |
| | CTTCTCAACTATATCCTAA |
| | ORF Start: ATG at 1 ORF Stop: TAA at 1699 |
| | SEQ ID NO: 300 566 aa MW at 62488.6kD |
| NOV24a, | MAGPGKEGVVWWEEKSMGQLREEDNIELNEEGRPVQTSRPSPPLCDCHCCGLPKRYII |
| CG153033-01 | |
| Protein Sequence | AIMSGLGFCISFGIRCNLGVAIVEMVNNSTVYVDGKQTAQFNWDPETVGLIHGSFFWG |
| | YIMTQIPGGFISNKFAANRVFGAAIFLTSTLNMFIPSAARVHYGCVMCVRILQGLVGV |
| | TYPACHGMWSKWAPPLERSRLATTSFCGSYAGAWAMPLAGVLVQYIGWSSVFYIYGM |
| | FGIIWYMFWLLQAYECPAAHPTISNEEKTYIETSIGEGANVVSLSVKFSTPWKRFFTS |
| | LPVYAIIVANFCRSWTFYLLLISQPAYFEEVFGFAISKVGLLSAVPHMVMTIVVPIGG |
| | QLADYLRSRQILTTTAVRKIMNCGGFGMEATLLLVVGFSHTKGVAISFLVLAVGFSGF |
| | AISGFNVNHLDIAPRYASILMGISNGVGTLSGMVCPLIVGANTRHKTREEWQNVFLIA |
| | ALVHYSGVIFYGVFASGEKQEWADPENLSEEKCGIIDQDELAEEIELNHESFASPKKK |
| | MSYGATSQNCEVQKKEWKGQRGATLDEEELTSYQNEERNFSTIS |

Further analysis of the NOV24a protein yielded the following properties shown in Table 24B.

TABLE 24B
Protein Sequence Properties NOV24a

| | |
|---|---|
| PSort | 0.6000 probability located in plasma membrane; 0.4000 |
| analysis: | probability located in Golgi body; 0.3000 probability located |
| | in endoplasmic reticulum (membrane); 0.3000 probability |
| | located in microbody (peroxisome) |
| SignalP | No Known Signal Sequence Predicted |
| analysis: | |

A search of the NOV24a protein against the Geneseq database, a proprietary database that contains sequences published in patents and patent publication, yielded several homologous proteins shown in Table 24C.

TABLE 24C
Geneseq Results for NOV24a

| | | NOV24a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAU99329 | Human transporter protein - Homo | 4 . . . 566 | 553/575 (96%) | 0.0 |
| | sapiens, 589 aa. [US2002082190- | 16 . . . 589 | 555/575 (96%) | |
| | A1, 27 JUN. 2002] | | | |
| ABB07689 | Rat glutamate transporter VGLUT3 | 4 . . . 566 | 509/580 (87%) | 0.0 |
| | amino acid sequence - Rattus sp, | 24 . . . 601 | 532/580 (90%) | |
| | 860 aa. [WO200208384-A2, 31 | | | |
| | JAN. 2002] | | | |
| AAM79273 | Human protein SEQ ID NO 1935 - | 4 . . . 530 | 413/542 (76%) | 0.0 |
| | Homo sapiens, 582 aa. | 11 . . . 549 | 473/542 (87%) | |
| | [WO200157190-A2, 09 AUG. | | | |
| | 2001] | | | |
| AAO13870 | Human polypeptide SEQ ID NO | 24 . . . 528 | 404/514 (78%) | 0.0 |
| | 27762 - Homo sapiens, 567 aa. | 38 . . . 551 | 450/514 (86%) | |
| | [WO200164835-A2, 07 SEP. 2001] | | | |
| AAW70500 | Human sodium-lithium | 24 . . . 528 | 403/514 (78%) | 0.0 |
| | countertransporter BNPI - Homo | 31 . . . 544 | 449/514 (86%) | |
| | sapiens, 560 aa. [WO9838203-A1, | | | |
| | 03 SEP. 1998] | | | |

In a BLAST search of public sequence datbases, the NOV24a protein was found to have homology to the proteins shown in the BLASTP data in Table 24D.

TABLE 24D
Public BLASTP Results for NOV24a

| | | NOV24a | Identities/ | |
|---|---|---|---|---|
| Protein | | Residues/ | Similarities | |
| Accession | | Match | for the | Expect |
| Number | Protein/Organism/Length | Residues | Matched Portion | Value |
| CAD30553 | Vesicular glutamate transporter 3 - | 4 . . . 566 | 553/575 (96%) | 0.0 |
| | Homo sapiens (Human), 589 aa. | 16 . . . 589 | 555/575 (96%) | |
| CAD37138 | Vesicular glutamate transporter 3 - | 4 . . . 566 | 510/575 (88%) | 0.0 |
| | Rattus norvegicus (Rat), 588 aa. | 16 . . . 588 | 533/575 (92%) | |
| Q9JI12 | Differentation-associated Na- | 4 . . . 561 | 421/573 (73%) | 0.0 |
| | dependent inorganic phosphate | 11 . . . 579 | 487/573 (84%) | |
| | cotransporter - Rattus norvegicus | | | |
| | (Rat), 582 aa. | | | |
| Q920B7 | Vesicular glutamate transporter 2 - | 4 . . . 530 | 417/542 (76%) | 0.0 |
| | Mus musculus (Mouse), 582 aa. | 11 . . . 549 | 475/542 (86%) | |
| CAD52142 | SI: PACKT73.2 (novel protein similar | 2 . . . 530 | 418/545 (76%) | 0.0 |
| | to solute carrier family 17 (sodium- | 8 . . . 550 | 472/545 (85%) | |
| | dependent inorganic phosphate | | | |
| | cotransporter), member 6 | | | |
| | (SLC17A6)) - Brachydanio rerio | | | |
| | (Zebrafish) (Danio rerio), 584 aa. | | | |

PFam analysis predicts that the NOV24a protein contains the domains shown in the Table 24E.

TABLE 24E
Domain Analysis of NOV24a

| | | Identities/ | |
|---|---|---|---|
| | NOV24a | Similarities | |
| | Match | for the | Expect |
| Pfam Domain | Region | Matched Region | Value |
| sugar_tr | 64 . . . 488 | 72/506 (14%) | 0.04 |
| | | 262/506 (52%) | |

### Example 25

The NOV25 clone was analyzed, and the nucleotide and encoded polypeptide sequences are shown in Table 25A.

TABLE 25A
NOV25 Sequence Analysis

| | SEQ ID NO:301 | 3374 bp | |
|---|---|---|---|
| NOV25a, | GCAATCATGAAGGACAGCGGGGACTCCAAGGACCAGCAACTCATGGTGGCGCTTCGGG | | |
| GG153818-01 | | | |
| DNA Sequence | TCCGGCCCATCAGCGTGGCAGAGCTGCAGCAAGGAGCTACCCTCATCGCCCATAAAGT | | |
| | GGATGAGCAGCATTTACCTGCTGCCACCCCCCTCTGCTCCCGGGGTGCTGTAGAGCCA | | |
| | GGCTCAAAGCTGCAAAGGGCCACTGGAGCAGTTCCCTCACAGCCCTCTCAGCTGCGAG | | |
| | TGGAGATCCCCAAGCCCAGCGTGCTGACCTCATCCCTCACCCAGCTGCCTGTGCCTCT | | |
| | TTGCTCTGTCCCAGGCTCTGCCCTGGAGGCGGCCCGGGGTTCCCAGGTGACCGTGGGC | | |
| | CTCCCTCTGGGGACCTTGCAGATGGTGGTTCTCATGGACCCAATGGAGGATCCCGACG | | |
| | ACATCCTGCGGGCGCATCGCTCCCGGCAGAAGTCCTACCTGTTCGACGTGGCCTTTGA | | |
| | CTTCACCGCCACCCAGGAGATGGTGTATCAGGCCACCACCAAGAGCCTCATCGAGGGC | | |
| | GTCATCTCAGGCTACAATGCCACTGTCTTTGCCTATGGCCCACAGGTAAGGGGAATGC | | |
| | CAGACTTGTGCGAGACAGCAATGATCTGCTGTCGGAAAACCTACACCATGCTGGGCAC | | |
| | AGACCAGGAGCCTGGCATCTATGTTCAGACCCTCAACGACCTCTTCCGTCCCATCGAG | | |
| | GAGACCAGCAATGACATGGAGTATGAGGTCTCCATGTCCTACCTGGAGATCTACAATG | | |
| | AGATGATCCGCGACCTGCTGAACCCCTCCCTGGGCTACCTGGAGCTGCGGGAGGACTC | | |
| | TAAGGGGGTGATCCAGGTGGCCGGCATCACCGAAGTCTCCACCATCAATGCCAAGGAG | | |
| | ATCATGCAGCTGCTGATGAAGGGGAACCGGCAGAGGACCCAGGAGCCCACGGCCGCCA | | |
| | ACCAGACGTCCTCCCGCTCCCACGCGGTACTGCAGGTGACCGTGCGCCAGCGCAGCCG | | |
| | GGTCAAGAACATCTTGCAGGAGGCGCAGGGCCGCCTGTTCATGATCGACCTGGCTGGC | | |
| | TCAGAGCGCGCCTCGCAGACACAGAATCGTGGGCAGCGTATGAAGGAGGGGGCCCACA | | |
| | TCAACCGCTCACTGCTGCCACTGGGCAACTGCATCAACGCCCTGAGCGACAAGGGTAG | | |
| | CAACAAGTACATCAACTATCGCGACAGCAAGCTCACCCGGCTCCTGAAGGACTCTCTG | | |
| | GGAGGAAACAGCCGCACAGTCATGATCGCTCACATCAGTCCTGCGAGCAGTGCCTTCG | | |
| | AGGAGTCCCGGAACACCCTGACCTACGCCGGCCGGGCCAAGAACATTAAGACTAGGGT | | |
| | GAAGCAGAACCTCCTGAACGTCTCCTACCACATCGCCCAGTACACCAGCATCATCGCT | | |
| | GACCTGCGGGGCGAGATCCAGCGACTCAAGCGCAAGATTGATGAGCAGACTGGGCGGG | | |
| | GCCAGGCCCGGGGCCGGCAGGATCGGGGTGACATCCGCCACATCCAAGCTGAGGTCCA | | |
| | GCTGCACAGCGGGCAGGGTGAGAAGGCTGGCATGGGACAGCTTCGGGAGCAGCTCGCC | | |
| | AGCGCCTTCCAGGAGCAGATGGATGTGCGGAGGCGCCTGCTGGAGCTGGAGAACCGCG | | |
| | CCATGGAGGTCCAGATTGACACCTCCCGACACCTGCTCACCATCGCCGGCTGGAAGCA | | |
| | TGAGAAGTCCCGCCGGGCCCTCAAATGGCGGGAGGAGCAGCGAAAGGAGTGCTACGCT | | |
| | AAGGACGACAGCGAGAAGGACTCAGACACAGGTGATGACCAACCAGACATCCTGGAGC | | |
| | CACCCGAGGTGGCCGCAGCCCGGGAGAGCATTGCAGCCCTGGTGGACGAGCAGAAGCA | | |
| | ACTGCGCAAGCAGAAGGTGTCCAGGGTTTGGGGGGACAAGGAGAGTGGGTTTAGGGGA | | |
| | CAGGATGCTGACCTGCGCCTCCTGCAGCTGGCGCTGGAGCAGCGCTGCCGGGAGCTGC | | |
| | GCGCGCGGGGCCGGCGCCTGGAGGAGACGCTGCCGCGGCGCATCGGCTCCGAGGAGCA | | |
| | GCGCGAGGTGCTCAGCCTGCTGTGCCGCGTGCACGAGCTCGAGGTGGAGAACACCGAG | | |
| | ATGCAGTCGCACGCGCTGCTCCGCGACGGTGCGCTCCGCCACCGCCACGAGGCCGTGC | | |
| | GCCGCCTGGAGCAGCACCGCAGTCTCTGCGACGAGATTATCCAGGGCCAGCGGCAGAT | | |
| | CATCGACGCAGACTACAACCTGGCCGTCCCGCAGCGCCTGGAAGAGCTCTACGAAGTG | | |
| | TACCTGCGGGAGCTGGAGGAGGGCAGCCTGGAGCAGGCCACCATCATGGACCAAGTGG | | |
| | CCTCCAGGGCCCTGCAGGACAGCTCCTTGCCCAAAATTACCCCAGCAGGAACCTCACT | | |
| | GACCCCAGATTCTGACCTGGAGAGTGTGAAGACATTGAGCTCTGATGCCCAGCACCTG | | |
| | CAGAACAGCGCCCTCCCTCCCCTCAGCACAGAGAGTGAAGGCCACCACGTGTTCAAGG | | |
| | CTGGTACTGGGGCCTGGCAGGCAAPAAGCTCCTCTGTGCCCACCCCACCTCCCATCCA | | |
| | GCTCGGCAGCCTGGTGACGCAGGAGGCCCCGGCTCAGGACAGCCTGGGCAGCTGGATC | | |
| | AACTCTTCCCCTGACAGCAGTGAGAACCTGTCGGAGATCCCCTTGTCCCACAAAGAGA | | |
| | GGAAGGAGATCCTGACTGGCACCAAGTGCATCTGGGTGAAGGCCGCCCGGCGGCGCTC | | |
| | GCGGGCCCTGGGAACCGAGGGGCGACACCTGCTGGCACCCGCGACAGAGCGCAGCAGC | | |
| | CTGTCCCTGCACTCACTGAGCGAGCCCGACGATGCGCGGCCACCAGGCCCACTGGCCT | | |
| | GCAACCGGCCGCCCAGCCCCACACTACAGCATGCTGCCAGTGAGGACAACCTGTCCAG | | |
| | CAGCACGGGCGAGGCCCCGTCCCGGGCAGTCGGACATCATGGGGACGGCCCCAGGCCC | | |
| | TGGCTGCGTGGCCAGAAGAAAAGCCTGGGCAAGAAAAGGGAGGAGTCGCTGGAGGCAA | | |
| | AGAGAAGGAAGCGGAGGTCCCGATCCTTCGAGGTCACCGGGCAAGGGCTCTCCCACCC | | |
| | CAAGACACACCTCCTGGGGCCCCATCAGGCGGAGCGCATCTCGGACCACAGGATGCCA | | |
| | GTGTGCAGGCACCCAGCCCCTGGTATCCGGCATCTGGGAAAGGTCACGCTACCTTTGG | | |
| | CCAAAGTCAAACTCCCTCCAAGCCAGAACACGGGCCCGGGGGACTCCTCACCCCTGGC | | |
| | TGTTCCCCCCAACCCAGGTGGTGGTTCTCGACGGGCTACCCGTGGGCCCCGCCTGCCC | | |
| | CATGGCACAAGCACCCATGGCAAAGATGGATGCTCCCGGCATAACTGAGGGGGCCTGC | | |
| | CTGGAACTGG | | |
| | ORF Start: ATG at 7 | | ORF Stop: TGA at 3352 |
| | SEQ ID NO:302 | 1115 aa | MW at 123442.0 kD |

| | |
|---|---|
| NOV25a, | MKDSGDSKDQQLMVALRVRPISVAELEEGATLIAHKVDEQHLPAATPLCSRGAVEPGS |
| CG153818-01 | |
| Protein Sequence | KLQRATGAVPSQPSQLRVEIPKPSVLTSSLTQLPVALCSVPGSALEGARGSQVTVGLP |
| | LGTLQMVVLMDPMEDPDDILRAHRSREKSYLFDVAFDFTATQEMVYQATTKSLIEGVI |
| | SGYNATVFAYGPQVRGMPDLCETAMICCGKTYTMLGTDQEPGIYVQTLNDLFRAIEET |
| | SNDMEYEVSMSYLEIYNEMIRDLLNPSLGYLELREDSKGVIQVAGITEVSTINAKEIM |
| | QLLMKGNRQRTQEPTAANQTSSRSHAVLQVTVRQRSRVKNILQEAQGRLFMIDLAGSE |
| | RASQTQNRGQRMKEGAHINRSLLALGNCINALSDKGSNKYINYRDSKLTRLLKDSLGG |
| | NSRTVMIAHISPASSAFEESRNTLTYAGRAKNIKTRVKQNLLNVSYHIAQYTSIIADL |
| | RGEIQRLKRKIDEQTGRGQARGRQDRGDIRHIQAEVQLHSGQGEKAGMGQLREQLASA |
| | FQEQMDVRRRLLELEMRANEVQIDTSRHLLTIAGWKHEKSRRALKwREEQRKECYAKD |
| | DSEKDSDTGDDQPDILEPPEVAAARESIAALVDEQKQLRKQKVSRVWGDKESGFRGQD |
| | ADLRLLQLALEQRCRELRARGRRLEETLPRRIGSEEQREVLSLLCRVHELEVENTEMQ |
| | SHALLRDGALRHRHEAVRRLEQHRSLCDEIIQGQRQIIDADYNLAVPQRLEELYEVYL |
| | RELEEGSLEQATIMDQVASRALQDSSLPKITPAGTSLTPDSDLESVKTLSSDAQHLQN |
| | SALPPLSTESEGHHVFKAGTGAWQAKSSSVPTPPPIQLGSLVTQEAPAQDSLGSWINS |
| | SPDSSENLSEIPLSHKERKEILTGTKCIWVKAARRRSRALGTEGRHLLAPATERSSLS |
| | LHSLSEGDDARPPGPLACKRPPSPTLQHAASEDNLSSSTGEAPSRAVGHHGDGPRPWL |
| | RGQKKSLGKKREESLEAKRRKRRSRSFEVTGQGLSHPKTHLLGPHQAERISDHRMPVC |
| | RHPAPGIRHLGKVTLPLAKVKLPPSQNTGPGDSSPLAVPPNPGGGSRRATRGPRLPHG |
| | TSTHGKDGCSRHN |

Further analysis of the NOV25a protein yielded the following properties shown in Table 25B.

TABLE 25B
Protein Sequence Properties NOV25a

| | |
|---|---|
| PSort | 0.9800 probability located in nucleus; 0.3000 |
| analysis: | probability located in microbody (peroxisome); |
| | 0.1000 probability located in mitochondrial matrix |
| | space; 0.1000 probability located in lysosome (lumen) |
| SignalP | No Known Signal Sequence Predicted |
| analysis: | |

A search of the NOV25a protein against the Geneseq database, a proprietary database that contains sequences published in patents and patent publication, yielded several homologous proteins shown in Table 25C.

TABLE 25C
Geneseq Results for NOV25a

| | | NOV25a | Identities/ | |
|---|---|---|---|---|
| | | Residues/ | Similarities | |
| Geneseq | Protein/Organism/Length | Match | forthe | Expect |
| Identifier | [Patent #, Date] | Residues | Matched Region | Value |
| AAO21658 | Protein fragment of the motor | 111 . . . 442 | 289/332 (87%) | e−155 |
| | domain HsKip3b - Homo sapiens, | 1 . . . 299 | 289/332 (87%) | |
| | 299 aa. [US6368841-B1, 09 APR. | | | |
| | 2002] | | | |
| AAM50137 | Human kinesin motor protein | 111 . . . 442 | 289/332 (87%) | e−155 |
| | HsKip3b motor domain - Homo | 1 . . . 299 | 289/332 (87%) | |
| | sapiens, 299 aa. [US6294371-B1, | | | |
| | 25 SEP. 2001] | | | |
| ABB64748 | Drosophila melanogaster | 140 . . . 816 | 259/692 (37%) | e−106 |
| | polypeptide SEQ ID NO 21036 - | 68 . . . 684 | 379/692 (54%) | |
| | Drosophila melanogaster, 728 aa. | | | |
| | [WO200171042-A2, 27 SEP. 2001] | | | |
| ABB07410 | Human kinesin motor protein, | 140 . . . 483 | 161/346 (46%) | 3e−81 |
| | HsKip3A - Homo sapiens, 864 aa. | 64 . . . 395 | 229/346 (65%) | |
| | [WO200196593-A2, 20 DEC. 2001] | | | |
| AAU76957 | Novel human kinesin motor protein, | 140 . . . 537 | 171/400 (42%) | 3e−79 |
| | HsKip3d - Homo sapiens, 898 aa. | 68 . . . 444 | 254/400 (62%) | |
| | [WO200212268-A1, 14 FEB. 2002] | | | |

In a BLAST search of public sequence datbases, the NOV25a protein was found to have homology to the proteins shown in the BLASTP data in Table 25D.

TABLE 25D
Public BLASTP Results for NOV25a

| | | NOV25a | Identities/ | |
|---|---|---|---|---|
| Protein | | Residues/ | Similarities | |
| Accession | | Match | for the | Expect |
| Number | Protein/Organism/Length | Residues | Matched Portion | Value |
| BAC04386 | CDNA FLJ37300 fis, clone | 90 . . . 637 | 510/549 (92%) | 0.0 |
| | BRAMY2015782, moderately | 11 . . . 544 | 512/549 (92%) | |
| | similar to KINESIN-LIKE | | | |
| | PROTEIN - Homo sapiens | | | |
| | (Human), 548 aa. | | | |
| Q9VFN0 | CG9913 protein - Drosophila | 140 . . . 816 | 259/692 (37%) | e−105 |
| | melanogaster (Fruit fly), 728 aa. | 68 . . . 684 | 379/692 (54%) | |
| CAD49067 | Kinesin, putative - Plasmodium | 121 . . . 478 | 191/363 (52%) | 4e−95 |
| | falciparum, 1669 aa. | 955 . . . 1304 | 252/363 (68%) | |
| O14343 | Kinesin-like protein 5 - | 7 . . . 486 | 195/485 (40%) | 1e−83 |
| | Schizosaccharomyces pombe | 2 . . . 437 | 276/485 (56%) | |
| | (Fission yeast), 883 aa. | | | |
| Q9SCJ4 | Kinesin-like protein - Arabidopsis | 89 . . . 716 | 217/631 (34%) | 4e−83 |
| | thaliana (Mouse-ear cress), 813 aa. | 13 . . . 548 | 338/631 (53%) | |

PFam analysis predicts that the NOV25a protein contains the domains shown in the Table 25E.

TABLE 25E
Domain Analysis of NOV25a

| | | Identities/ | |
|---|---|---|---|
| | NOV25a | Similarities | |
| | Match | for the | Expect |
| Pfam Domain | Region | Matched Region | Value |
| kinesin | 140 . . . 186 | 22/54 (41%) | 2.1e−10 |
| | | 38/54 (70%) | |
| kinesin | 203 . . . 468 | 126/319 (39%) | 2.3e−89 |
| | | 212/319 (66%) | |

### Example 26

The NOV26 clone was analyzed, and the nucleotide and encoded polypeptide sequences are shown in Table 26A.

TABLE 26A
NOV26 Sequence Analysis

| | SEQ ID NO:303 | 13734 bp | |
|---|---|---|---|
| NOV26a, | GTTTCTTGCACTCTTAACAGATACACAGTGTAAGGAAAGGCCAAGATGACAATGGCCC | | |
| CG154435-01 | | | |
| DNA Sequence | CGGACGTCAGACTAGAGTATCTGGAGGAAGTTGCCTCCATCGTCCTGAAGTTCAAGCC | | |
| | GGACAAGTGGAGCAAGCTGATAGGCGCCGAGGAGAACGTGGCCCTGTTCACAGAGTTC | | |
| | TTTGAAAAGCCCGACGTCCAGGTGCTGGTGCTGACGCTCAATGCAGCCGGCATGATCA | | |
| | TACCCTGCCTGGGCTTCCCCCAGTCCCTCAAGTCCAAAGGGGTTTACTTCATCAAGAC | | |
| | AAAGTCCGAGAACATCAACAAGGACAACTACAGGGCCCGGCTCCTTTACGGCGACATC | | |
| | AGCCCCACACCCGTGGACCAGCTGATCGCGGTGGTGGAGGAGGTCCTCTCTTCTCTGT | | |
| | TAAACCAAAGTGAGAACATGGCTGGATGGCCCCAGGTGGTCTCGGAAGACATCGTGAA | | |
| | GCAGGTCCACAGGCTGAAGAATGAAATGTTTGTGATGAGTGGCAAGATCAAAGGCAAA | | |
| | ACCTTGCTGCCTATTCCGGAGCACCTGGGCAGCCTGGATGGCACGCTGGAGTCCATGG | | |
| | AGAGGATCCCCTCTTCACTGGACAACTTGCTCCTOCACGCCATTGAAACCACCATCAT | | |
| | CGACTGGTCCCACCAGATCCGGGATGTGCTGAGCAAAGACTCAGCCCAGGCGCTGCTG | | |
| | GATGGGCTGCACCCCCTGCCCCAAGTGGAGTTCGAGTTCTGGGACACTCGGCTGCTGA | | |
| | ACCTCAAGTGCATCCATGAACAGCTAAACAGACCCAAAGTGAACAAGATTGTTGAGAT | | |
| | CCTAGAGAAAGCCAAAAGCTGCTACTGGCCAGCCCTGCAPAACGTTTACACCAACGTC | | |
| | ACTGAAGGGCTGAAGGAAGCCAACGACATCGTGCTCTATTTGAAGCCCCTACGGATCC | | |
| | TGCTGGAGGAGATGGAACAAGCCGACTTCACGATGCTCCCCACCTTCATTGCCAAGGT | | |
| | GCTGGACACCATCTGCTTCATCTGGGCCACCTCTGAGTACTATAACACACCTGCCAGG | | |
| | ATCATCGTCATCCTGCAGGAGTTCTGCAACCAAATCATCGAGATGACACGAACCTTCC | | |
| | TGAGCCCGGAAGAGGTGCTGAAGGGCCTGCAAGGTGAAATCGAGGAAGTCCTGAGTGG | | |
| | CATCTCCCTGGCTGTAAATGTGCTGAAGGAGCTCTACCAGACGTACGACTTCTGCTGC | | |
| | GTGAACATGAAGCTTTTCTTTAAGGACAAAGAGCCCGTGCCTTGGGAATTCCCTTCTT | | |
| | CTCTTGCCTTTTCCAGGATAAATTCCTTCTTCCAGCGCATCCAGACCATTGAGGAACT | | |
| | CTATAAAACAGCAATTGAGTTTCTGAAGCTGGAGAAAATCGAGCTTGGGGGCGTGCGT | | |
| | GGGAACCTCCTCGGGAGCCTGGTGACCCGTATCTATGATGAGGTCTTTGAGCTGGTGA | | |
| | AGGTTTTTGCCGACTGCAAATATGATCCCTTGGACCCTGGAGACTCGAATTTTGACCG | | |
| | TGATTATGCTGATTTTGAGATCAAAATCCAAGACCTGGATAGGAGGCTGGCCACGATC | | |
| | TTTTGCCAAGGATTTGATGACTGCAGCTGTATCAAGTCCTCCGCAAAGCTCCTGTACA | | |
| | TGTGTGGGGGCCTCATGGAGCGGCCCCTGATTCTTGCCGAGGTGGCGCCCAGGTATTC | | |
| | AGTCATGCTGGAGCTGTTTGACGCTGAGCTAGACAATGCTAAGATCTTGTACGATGCC | | |
| | CAGATGGCGGCCTCCGAGGAGCGGAACATCCCCCTGATCCACAAAAACATGCCTCCCG | | |
| | TGGCCGGGCAGCTCAAATGGACCCTGGAGCTGCAGGAGAGGCTAGAGGTGTCCATGAA | | |
| | ACACCTGAAGCACGTCGAACACCCGGTCATGTCTGGAGCAGAGGCCAAGCTGACCTAT | | |
| | CAGAAGTATGACGAGATGATGGAGCTGCTGAGGTGCCACCGCGAGAAGATCTACCAGC | | |
| | AGTGGGTGGCGGGCGTGGACCAGGACTGCCACTTTAACCTGGGGCAGCCGCTGATTCT | | |
| | GCGGGACGCCGCTAGCAACCTCATCCACGTCAACTTCAGCAAAGCGTTGGTGGCAGTT | | |
| | CTGAGAGAAGTCAAGTATTTGAATTTCCAGCAACAGAAAGAGATTCCAGACAGTGCGG | | |
| | AGAGTCTGTTCTCAGAGAACGAAACTTTCCGGAAGTTTGTGGGCAACCTGGAGCTCAT | | |
| | CGTTGGCTGGTATAATGAGATAAAGACTATAGTGAAGGCAGTAGAATTTCTACTAATA | | |
| | AAGTCAGAACTGGAAGCAATTGATGTCAAGTTATTGAGCGCTGAAACGACATTATTCT | | |
| | GGAATGGCGAAGGTGTGTTTCAGTACATTCAAGAGGTGCGAGAAATTCTGCACAACTT | | |
| | GCAGAACAGGATGCAAAAGGCAAAACAAAATATAGAAGGAATTTCCCAGGCTATGAAG | | |
| | GACTGGTCGGCCAACCCGCTGTTTGAAAGAAAGGACAATAAGAAAGAGGCCCTGTTAG | | |
| | ACTTGGATGGAAGAATTGCCAACCTCAACAAGCGCTACGCAGCAGTCAGGGATGCTGG | | |
| | AGTGAAGATCCAACCCATGGAAAACGCAGAACTATTCAGGGCAGACACACTGAGCCTG | | |
| | CCCTGGAAGGATTATGTCATCTACATTGACGACATGGTCTTAGATGAATTTGACCAGT | | |
| | TCATTCGCAAATCTCTGAGTTTCCTAATGGACAACATGGTTATAGATGAGAGTATCGC | | |
| | TCCCCTGTTTGAGATCCGCATGGAGCTGGACGAGGATGGGCTGACCTTCAACCCGACC | | |
| | CTGGAGGTGCGCTCAGATCGCGGCTTCCTGGCACTGATCGAGGGCCTGGTCAACGACA | | |
| | TCTACAACGTAGCCAGGCTCATCCCTCGGCTGGCCAAGGACAGGATGAACTACAAGAT | | |
| | GGACCTGGAAGATAACACAGACCTCATAGAGATGAGGGAGGAGGTGTCCAGCCTGGTC | | |
| | ATCAATGCCATGAAGGAGGCCGAGGAGTACCAGGATTCCTTTGAGAGGTACTCCTACC | | |
| | TCTGGACGGACAACCTGCAGGAGTTTATGAAGAATTTCCTGATATATGGGTGTGCAGT | | |
| | CACTGCGGAGGACTTGGACACCTGGACAGATGACACCATCCCCAAGACACCGCCCACC | | |
| | CTGGCTCAGTTCCAGGAGCAGATCGACTCCTACGAGAAGCTGTATGAGGAGGTGTCCA | | |
| | AGTGCGAGAACACCAAGGTGTTCCACGGCTGGCTGCAGTGCGACTGCCGCCCCTTCAA | | |
| | GCAGGCCCTGCTCAGCACAATCCGGCGCTGGGGCTTCATGTTCAAGCGGCACCTGAGC | | |
| | AACCACGTCACCAACAGCCTGGCTGACCTGGAAGCCTTCATGAAAGTCGCCAGAATGG | | |
| | GCTTGACCAAGCCCCTCAAGGAGGGGGACTATGATGGGCTTGTGGAGGTGATGGGGCA | | |
| | CCTGATGAAAGTCAAGGAGAGGCAAGCAGCCACCGACAACATGTTTGAGCCCCTGAAG | | |
| | CAAACCATCGAGCTGCTCAAGACCTACGGGGAGGAGATGCCAGAGGAGATCCACTTGA | | |
| | AGCTGCAGGAGCTGCCGGAGCACTGGGCAAATACCAAGAAACTGGCCATTCACGTGAA | | |
| | GCTGACCGTGGCACCACTCCAGGCCAACGAGGTCAGCATCCTGCGGCGGAAATGCCAG | | |
| | CAATTCGACCTCAAGCAACATGAGTTCAGGGAGAGGTTCAGGCGCGAGGCCCCGTTCT | | |
| | CCTTCAGCGACCCCAACCCCTACAAGTCCCTGAATAAGCAACAAAAGAGCATCTCCGC | | |
| | CATGGAAGGCATCATGGAGGCGCTGTCCAAGTCCGGGGGCCTGTTCGAGGTCCCCGTC | | |
| | CCAGACTACAAGCAGCTCAAGGCCTGCCACCGGGAGGTCCGCCTACTGAAGGAGCTCT | | |
| | GGGACATCGTTGTTGTGGTAAATACCAGCATCGAGGACTGGAAGACCACCAAGTGGAA | | |
| | AGATATCAACGTTGAGCAGATGGACATAGATTGTAAGAAGTTTGCCAAGGACATGAGG | | |
| | TCTTTGGACAAGGAGATGAAAACCTGGGATGCCTTCGTGGGGCTCGACAACACCGTGA | | |
| | AAAACGTGATCACGTCCCTGCGTGCCGTGAGCGAGCTGCAGAACCCTGCCATTCGGGA | | |
| | ACGCCACTGGCAGCAGCTCATGCAGGCCACCCAGGTGAAATTTAAAATGTCAGAAGAG | | |
| | ACGACCCTGGCAGATTTACTCCAGCTGAACCTCCACAGTTACGAGGATGACGTCCGCA | | |
| | ACATCGTGGACAAGGCCGTGAAGGAGTCGGGCATGGAAAAGGTGCTGAAAGCCCTGGA | | |
| | CAGTACCTGGAGCATGATGGAATTCCAGCACGAGCCGCACCCGCGGACAGGCACCATG | | |
| | ATGCTCAAGTCCAGCGAGGTGCTGGTGGAGACGCTGGAGGACAACCAGGTGCAGCTGC | | |
| | AGAACCTGATGATGTCCAAGTACCTGGCCCACTTCCTGAAGGAGGTGACAAGCTGGCA | | |
| | GCAGAAGCTGTCCACGGCGGACTCCGTCATCTCCATCTGGTTTGAGGTCCAGCGAACC | | |
| | TGGAGCCACCTGGAGAGCATCTTCATCGGCTCCGAAGACATCCGCACCCAGCTCCCGG | | |
| | GGGACTCCCAGCGCTTTGACGACATCAACCAGGAATTCAAGGCCTTGATGGAAGATGC | | |
| | AGTGAAAACACCCAACGTGGTGGAAGCCACCAGCAAACCCGGCCTCTACAATAAACTG | | |
| | GAGGCCCTGAAGAAGAGCTTGGCCATCTGTGAAAAGGCTTTGGCAGAGTATTTAGAGA | | |
| | CGAAAAGACTGGCTTTCCCCCGGTTCTATTTTGTCTCCTCGGCTGACCTCCTGGACAT | | |
| | TCTCTCCAATGGCAATGACCCCGTGGAGGTGAGCCGCCACCTGTCCAAACTCTTCGAT | | |
| | AGCCTGTGTAAACTGAAGTTCCGGCTCGATGCCAGTGACAAACCTCTCAAGGTGGGCC | | |
| | TGGGAATGTACAGCAAGGAGGACGAGTACATGGTTTTTGATCAGGAATGCGACCTCTC | | |
| | GGGGCAGGTGGAAGTGTGGCTGAATCGAGTGCTGGACCGAATGTGCTCTACCCTCCGG | | |
| | CACGAAATCCCAGAGGCCGTGGTGACCTACGAAGAGAAGCCGAGGGAGCAGTGCATCC | | |
| | TGGACTACCCAGCCCAGGTGGCCCTGACTTGCACCCAGATCTGGTGGACGACCGAGGT | | |
| | GGGCCTGGCATTTGCCAGGCTGGAGGAAGGCTATGAAAACGCTATCAGAGATTATAAC | | |
| | AAAAAGCAGATTAGCCAGCTGAACGTACTCATCACCCTGCTCATGGGGAACCTCAACG | | |
| | CTGGCGACAGGATGAAGATCATCACCATCTGCACCATCGATGTGCACGCACGGGACGT | | |
| | GGTGGCCAAAATGATCGTGGCCAAGGTGGAGAGTTCTCAGGCCTTCACCTGGCAGGCC | | |
| | CAGCTCCGGCATCGCTGGGACGAAGAGAAGCGACACTGCTTTGCCAACATCTGCGATG | | |
| | CCCAAATCCAGTATTCCTATGAGTATCTGGGCAACACGCCGCGGCTGGTCATCACCCC | | |
| | ACTCACTGACAGGTGCTATATCACCCTGACCCAGTCCCTCCATCTCATCATGGGTGGA | | |
| | GCCCCTGCCGGCCCCGCTGGGACCGGCAAGACTGAGACGACCAAGGACCTGGGCAGAG | | |
| | CCCTGGGCACCATGGTCTACGTCTTCAACTGCTCCGAGCAGATGGACTACAAGTCCTG | | |
| | TGGAAATATCTACAAGGGCCTGGCCCAGACGGGAGCCTGGGGCTGCTTTGACGAGTTT | | |
| | AATCGCATCTCAGTGGAAGTCTTGTCTGTGATTGCCGTGCAGGTAAAATGTGTCCAGG | | |
| | ATGCAATTCGGGCCAAGAAAAAAGCATTCAATTTCCTGGGAGAGATCATAGGCCTCAT | | |
| | TCCCACCGTCGGTATCTTCATCACCATGAACCCTGGGTACGCCGGACGCGCGGACCTG | | |
| | CCTGAGAACCTAAAAGCCTTATTCAGGCCCTGTGCCATGGTCGTCCCCGACTTCGAAC | | |
| | TGATATGTGAGATCATGCTCATGGCCGAGGGCTTTCTGGAAGCCCGCCTTCTGGCCAG | | |
| | GAAGTTCATCACCCTGTACACCTTGTGCAAGGAGCTGCTCTCGAAGCAGGATCATTAC | | |
| | GACTGGGGCCTGAGAGCCATCAAGTCTGTGCTGGTGGTGGCCGGCTCCCTGAAGAGGG | | |
| | GCGACCCCAGCCGGGCAGAGGACCAGGTGCTCATGCGGGCGCTGAGAGACTTCAACAT | | |
| | CCCCAAGATTGTGACAGACGACCTGCCCGTATTCATGGGACTGATCGGGGACCTCTTC | | |
| | CCGGCTCTGGACGTGCCTCGGAAACGGGACCTGAATTTTGAAAAGATCATCAAGCAGA | | |
| | GCATCGTGGAGCTCAAGCTGCAGGCGGAGGACAGCTTCGTGCTGAAGGTGGTGCAGCT | | |
| | GGAGGAGCTGCTGCAGGTCCGCCACTCCGTGTTCATCGTCGGGAATGCGGGCAGCGGC | | |
| | AAATCTCAGGTCCTCAAATCCCTCAACAAGACCTATCAGAACCTGAAGAGGAAGCCGG | | |
| | TCGCCGTGGACCTGGACCCCAAGGCCGTCACCTGCGACGAGCTCTTTGGCATCATCAA | | |
| | CCCAGTGACCAGGGAATGGAAAGATGGCCTGTTCTCCACCATCATGCGAGACCTGGCC | | |
| | AACATCACCCATGACGGCCCCAAGTGGATCATCCTTGACGGAGACATAGACCCCATGT | | |
| | GGATCGAGTCTCTCAACACAGTCATGGATGACAACAAGGTCCTCACCCTGCCCAGCAA | | |
| | CGAGCGGATCCCCCTGAACCGCACCATGAGGCTGGTGTTCGAAATCAGCCACCTGAGG | | |
| | ACGGCCACCCCAGCCACCGTTTCCAGAGCCGGCATCCTCTACATCAACCCAGCCGACC | | |
| | TGGGATGGAACCCGGTGGTGAGCAGCTGGATCGAGAGGCGCAAGGTGCAGTCGGAGAA | | |
| | GGCCAACCTGATGATCCTCTTTGACAAGTACCTGCCCACGTGCCTGGACAAGTTGCGC | | |
| | TTTGGGTTCAAGAAGATCACGCCAGTGCCGGAGATCACGGTGATCCAAACGATTCTGT | | |
| | ACCTGCTGGAGTGCCTGCTCACGGAGAAGACCGTGCCCCCCGACTCCCCCAGGGAGCT | | |
| | GTACGAGCTGTACTTCGTGTTCACCTGCTTCTGGGCCTTCGGTGGCGCCATGTTCCAG | | |
| | GACCAGCTTGTGGATTATCGAGTGGAGTTCAGTAAATGGTGGATCAACGAATTCAAGA | | |
| | CTATCAAGTTCCCCTCGCAGGGAACGATTTTTGACTACTACATTGATCCTGACACAAA | | |
| | AAAGTTCCTGCCCTGGACAGATAAAGTGCCCTCCTTTGAGCTCGATCCCGATGTCCCA | | |
| | CTGCAGGCCTCTTTGGTCCACACCACGGAAACCATCCGCATCCGCTACTTCATGGACC | | |
| | TGCTCATGGAGAAGTCCTGGCCGGTGATGCTGGTGGGGAACGCGGGGACGGGCAAGTC | | |
| | GGTGCTGATGGGGGACAAGCTGGAAAGCCTGAACACGGACAACTACCTGGTGCAGGCT | | |
| | GTGCCCTTCAACTTCTACACGACCTCAGCCATGCTGCAGGGGGTGCTGGAGAAGCCGC | | |
| | TGGAGAAGAAATCGGGGAGGAACTACGGGCCGCCAGGCACTAAGAAGCTCGTCTACTT | | |
| | CATCGACGACATGAACATGCCCGAGGTGGACAAGTATGGGACGGTGGCCCCGCACACC | | |
| | CTCATCCGGCAGCACATGGACCACCGGCACTGGTATGACAGACATAAGCTGACGTTAA | | |
| | AAGATATCCATAATTGTCAGTACGTGGCCTGCATGAACCCCACTTCCGGATCCTTCAC | | |
| | CATCGACTCCAGGCTTCAGCGCCATTTCTGCGTGTTTGCTGTGAGCTTCCCCGGCCAG | | |
| | GAGGCCCTCACCACCATCTACAACACAATCCTGACGCAGCACCTGGCCTTCCGCTCGG | | |
| | TCTCCATGGCTATCCAGAGGATAAGCAGCCAGCTGGTGGCCGCGGCCCTGGCTTTGCA | | |
| | TCAGAAAATCACGGCAACATTTCTTCCCACGGCCATTAAGTTTCATTATGTCTTCAAC | | |
| | CTCAGGGACCTCTCCAATATTTTCCAGGGACTCTTATTTTCCACAGCAGAAGTTCTGA | | |
| | AAACCCCACTGGACCTCGTCCGCCTTTGGCTACATGAGACTGAACGAGTGTATGGTGA | | |
| | CAAAATGGTTGACGAAAAAGACCAGGAAACATTGCATACAGTCACCATGGCCTCCACC | | |
| | AAGAAGTTCTTTGATGATCTTGGTGATGAACTCTTATTTGCCAAGCCAAATATCTTCT | | |
| | GCCACTTTGCTCAACGGATTGGCGATCCCAAATATGTTCCTGTAACCGACATGGCTCC | | |
| | TCTGAACAAGCTCCTCGTGGACGTCCTGGACAGCTACAATGAAGTTAATGCAGTCATG | | |
| | AATTTCGTGCTGTTTGAGGACGCCGTGGCTCACATCTGCAGGATTAATCGCATCCTGG | | |
| | AGTCTCCCCGGGGGAATGCCCTGCTGGTGGGGGTGGGCGGCAGTGGCAAACAGAGCCT | | |
| | CTCCCGCCTGGCAGCGTACATCAGCGGGCTTGACGTGTTTCAGATCACCCTCAAGAAG | | |
| | GGCTACGGGATCCCCGACCTCAAGATTGACCTCGCTCCTCAGTACATAAAGGCTGCCG | | |
| | TGAAGAACGTTCCCTCGGTGTTCCTGATGACAGACTCCCAGGTGGCCGAGGAGCAGTT | | |
| | TCTGGTGCTGATCAATGACCTGCTGGCCTCAGGAGAGATCCCTGGGCTGTTTATGGAG | | |
| | GACGAGGTGGAGAACATCATCTCCTCCATGCGACCCCAAGTCAAGTCCCTTGGCATGA | | |
| | ATGACACTCGGGAAACATGTTGGAAGTTCTTCATCGAAAAAGTGCGCAGACAGCTCAA | | |
| | GGTGATCCTGTGTTTCTCCCCTGTGGGCTCCGTGCTCCGGGTACGAGCCAGAAAGTTC | | |
| | CCAGCTGTGGTCAACTGCACGGCCATCGACTGGTTCCACGAGTGGCCGGAAGATGCGC | | |
| | TGGTGTCCGTCAGCGCCCGCTTCCTGGAGGAGACTGAGGGGATTCCGTGGGAAGTCAA | | |
| | GGCCTCCATCAGCTTCTTCATGTCCTACGTGCACACCACCGTCAACGAGATGTCCAGG | | |
| | GTATACCTGGCTACTGAGAGGCGCTACAACTACACCACACCCAAAACCTTTCTGGAGC | | |
| | AGATCAAACTGTACCAGAACCTGCTGGCCAAGAACAGAACGGAACTTGTTGCCAAAAT | | |
| | CGAGAGGCTGGAGAACGGCCTGATGAAGCTGCAGAGCACGGCTTCCCAGGTGGATGAT | | |
| | TTGAAAGCCAAGTTCGCGATTCAGGAGGCTGAGCTCAAGCAGAAGAATGAGAGCCCAG | | |
| | ACCAACTGATCCAGGTGGTCGGCATCGAGGCCGAGAAGGTCACCAAAGAGAAGGCCAT | | |
| | TGCTGACCAGGAAGAAGTCAAGGTCGAGGTCATCAATAAGAACGTCACTGAGAAGCAA | | |
| | AAGGCCTGTGAAACAGACCTGGCCAAAGCAGAACCGGCCCTGCTGGCAGCCCAGGAGG | | |
| | CTCTGGACACTCTGAATAAGAACAACCTGACAGAGCTGAAGTCCTTTGGGTCCCCGCC | | |
| | GGATGCTGTGGTCAACGTCACCGCCGCCGTCATGATTCTGACCGCACCTCGGGGCAAG | | |
| | ATCCCCAAGGACAAGAGCTGGAAGGCGGCCAAGATCATGATGGGCAAGGTGGACACCT | | |
| | TCCTAGACTCCCTGAAGAAGTTCGACAAGGAGCACATCCCTGAGGCCTGCCTGAAGGC | | |
| | CTTCAAGCCCTACCAAGGCAACCCGACGTTCGACCCCGAGTTCATCCGCTCCAAGTCC | | |
| | ACGGCCGCCGCCGGCCTGTGCTCCTGGTGCATCAACATCGTCCGCTTCTACGAGGTCT | | |
| | ACTGCGACGTGGCGCCCAAGAGGCAGGCACTGGAGGAGGCTAATGCAGAGCTGGCAGA | | |
| | GGCACAAGAGAAGCTGTCCCGGATCAAAAACAAGATTGCCGAACTTAACGCCAACCTG | | |
| | AGCAACCTAACCTCAGCGTTTGAAAAAGCAACAGCTGAGAAAATCAAGTGTCAGCAAG | | |
| | AGGCCGATGCCACGAACAGGGTGATCTTACTGGCGAACAGGCTGGTCGGGGGATTAGC | | |
| | ATCGGAAAACATCCCCTGGGCTGAGTCTGTGGAGAACTTCAGGAGCCAGGGGGTCACG | | |
| | CTGTGTGGGGACGTCCTGCTCATCTCTGCCTTCGTGTCCTACGTGGGCTACTTCACCA | | |
| | AGAAATACCGGAATGAGCTGATGGAGAAATTCTGCATCCCTTACATACATAACTTAAA | | |
| | GGTCCCCATCCCGATCACGAATGGCCTGGATCCCTTGAGCCTGCTGACAGATGACGCG | | |
| | GACGTGGCCACCTGGAACAACCAGGGCCTCCCCAGCGACCGCATGTCCACCGAGAATG | | |
| | CCACCATCCTGGGCAACACCGAGCGGTGGCCGCTGATCGTGGACGCCCAGCTCCAAGG | | |
| | AATCAAGTGGATCAAAAACAAATACAGGAGTGAACTGAAAGCCATCCGCCTGGGACAG | | |
| | AAGAGCTACCTGGATGTCATCGAGCAGGCCATCTCGGAAGGGGACACCTTGCTCATTG | | |
| | AGAACATCGGCGAAACCGTGGACCCCGTGCTGGACCCTCTACTGGGCAGGAACACGAT | | |
| | TAAAAAGGGAAAGTACATTAAGATCGGTGACAAGGAGGTGGAGTACCACCCCAAGTTC | | |
| | CGCCTGATCCTACACACCAAGTACTTCAACCCACACTACAACCCAGAGATGCAGGCTC | | |
| | AGTGCACCCTCATCAACTTCCTGGTCACCAGGGATGGACTCGAGGACCAACTCTTGGC | | |
| | CGCTGTGGTGGCCAAAGAGCGCCCAGATCTGGAACAGCTGAAGGCAAACCTCACCAAG | | |
| | TCTCAAAACGAATTTAAGATTGTTCTGAAAGAGCTGGAAGATTCGCTCCTGGCCCGTC | | |
| | TGTCGGCTGCGTCGGGGAACTTTCTGGGAGACACGGCCTTGGTGGAGAATCTGGAGAC | | |
| | CACCAAGCACACAGCCAGCGAGATCGAGGAGAAGGTGGTGGAGGCAAAAATCACAGAA | | |
| | GTTAAAATCAACGAAGCGAGAGAGAACTACCGCCCGGCTGCGGAGAGGGCATCTCTGC | | |
| | TCTACTTCATACTGAACGATCTCAACAAAATCAACCCCGTCTACCAGTTCTCCCTCAA | | |
| | GGCCTTCAACGTGGTGTTTGAGAAAGCCATCCAGAGGACCACCCCTGCCAACGAGGTG | | |
| | AAGCAGCGGGTGATCAACCTGACGGACGAGATCACCTACTCCGTCTACATGTACACGG | | |
| | CCCGGGGACTCTTCGAGAGGGACAAACTCATTTTCCTGGCACAAGTTACGTTTCAGGT | | |
| | CCTGTCCATGAAGAAGGAGCTGAACCCAGTGGAGCTGGATTTCCTCCTGCGGTTCCCT | | |
| | TTTAAGGCCGGAGTGGTCTCACCAGTGGACTTCCTCCAGCATCAAGGCTGGGGCGGGA | | |
| | TCAAGGCCCTCTCCGAGATGGATGAGTTCAAAAATCTGGACAGTGACATCGAAGGATC | | |
| | TGCCAAGCGCTGGAAAAAGCTGGTGGAGTCGGAAGCCCCCGAGAAGGAGATCTTCCCC | | |
| | AAGGAGTGGAAGAACAAGACGGCCCTGCAGAAGCTGTGCATGGTGCGCTGCCTGCGGC | | |
| | CAGATCGCATGACCTACGCTATCAAGAACTTCGTGGAGGAAAAGATGGGCAGCAAGTT | | |
| | CGTGGAAGGCCGGAGTGTTGAGTTTTCTAAGTCCTACGAGGAGAGCAGCCCCTCCACG | | |
| | TCAATCTTCTTCATCCTCTCCCCGGGGGTTGACCCCTTGAAAGACGTGGAAGCCCTGG | | |
| | GAAAAAAACTAGGGTTTACCATAGACAATGGAAAACTCCATAATGTGTCCCTGGGGCA | | |
| | GGGACAAGAGGTGGTGGCTGAGAACGCCCTGGACGTGGCTGCAGAGAAAGGACACTGG | | |
| | GTCATTCTGCAGAATATCCACCTGGTGGCCCGGTGGCTGGGAACACTGGACAAGAAGC | | |
| | TGGAGTGCTACAGCACGGGCAGCCATGAGGACTACCGCGTGTTCATCAGCGCGGAGCC | | |
| | TGCCCCCAGCCCCGAGACCCACATCATCCCCCAGGGCATTCTGGAGAACGCCATCAAG | | |
| | ATCACCAACGAGCCCCCCACGGGCATGCACGCCAACTTGCACAAGGCCCTGGACCTGT | | |
| | TCACCCAGGACACCCTGGAGATGTGCACCAAGGAGATGGAGTTCAAGTGCATGCTCTT | | |
| | CGCCCTGTGCTACTTCCACGCTGTGGTGGCAGAGAGGCGCAAGTTCGGCGCCCAGGGC | | |
| | TGGAACCGGTCGTACCCCTTCAACAACGGGGACCTCACCATCTCCATCAACGTGCTCT | | |
| | ACAACTACCTGGAGGCCAACCCCAAGGTGCCCTGGGACGATCTCCGCTACCTTTTTGG | | |
| | TGAAATCATGTATGGCGGCCACATCACAGATGACTGGGACCGTCGGCTGTCCAGGACC | | |
| | TACCTGGCTGAATACATCCGGACGGAGATGCTGGAGGGAGACGTCCTGCTGGCCCCCG | | |
| | GCTTTCAGATCCCCCCCAACCTGGACTACAAGGGTTACCACGAATACATCGATGAGAA | | |
| | CCTGCCCCCTGAGAGTCCCTATCTGTATGGCCTGCACCCCAACGCAGAGATTGGCTTT | | |
| | CTGACGGTCACCTCAGAGAAGCTGTTCCGCACTGTCCTGGAAATGCAGCCAAAAGAGA | | |
| | CGGACTCGGGGGCAGGCACGGGACTGTCCCGCGAGGAGAAGGTGAAGGCCGTGCTGGA | | |
| | CGACATCCTGCAGAAGATTCCGGAGACTTTCAACATGGCTGAGATCATGGCAAAGGCA | | |
| | GCGGAAAAGACCCCCTACGTGGTAGTCGCCTTTCAAGAATGTGAAAGAATGAACATCC | | |
| | TGACCAACGAAATGCGCCGTTCGCTCAAGGAGCTGAACCTGGGGCTGAAGGGAGAACT | | |
| | GACCATCACGACCGACGTGGAAGATCTGTCCACGGCTCTCTTCTATGACACCGTGCCT | | |
| | GATACGTGGGTGGCCCGGGCCTACCCCTCCATGATGGGCCTGGCGGCCTGGTACGCAG | | |
| | ACCTGCTGCTCCGCATCAGGGAACTCGAGGCCTGGACGACAGACTTTGCCCTGCCCAC | | |
| | CACCGTGTGGCTGGCCGGCTTCTTCAACCCCCAGTCGTTCCTCACGGCCATCATGCAG | | |
| | TCCATGGCCAGGAAGAACGAGTGGCCCCTGGACAAGATGTGTCTGTCTGTCGAGGTGA | | |
| | CCAAGAAAAACCGAGAGGACATGACCGCTCCTCCGCGAGAGGGCTCCTACGTGTACGG | | |
| | ACTCTTCATGGAAGGGGCTCGCTGGGACACCCAGACTGGAGTCATCGCTGAAGCGCGG | | |
| | CTGAAAGAGCTGACCCCGGCCATGCCTGTCATCTTCATCAAGGCCATTCCTGTGGACC | | |
| | GCATGGAGACCAAGAACATCTATGAGTGTCCCGTGTACAAAACACGCATCCGCGGCCC | | |
| | CACCTATGTCTGGACCTTTAACTTGAAGACCAAAGAGAACGCAGCGAAGTGGATCCTG | | |
| | GCAGCCGTGGCGCTGCTCCTACAGGTTTAGCTCGCTCCTGCCTCACAGCCCACACTCC | | |
| | CTGGGGCTCOACCACAACTCAGCCCTTCACCTGTGCACCTGTGACTTATTCTTTACAG | | |
| | GAACTGGTGGTGGTTTTTCGTTCTCTTAAATAATCAGGTGCTTTGTAACCAAGCACAT | | |
| | CGGAACCAGAGGGTGGAGGTTGGTGTGGAAGAGGTGGGGCAGATTAAAGCCAGTGGAG | | |
| | CCACTCAGCTGTGCCCATCCATTCTGTGCCTGATGGCCACTGTGAGGCCTGGTTCAGG | | |
| | CTTTGGGGAAACGCCCCAATTCCCAGCAGCCAGAGGCAAGCATTCC | | |
| | ORF Start: at 61 | | ORF Stop: TAG at 13426 |
| | SEQ ID NO:304 | 4455 aa | MW at 508571.2 kD |

| | |
|---|---|
| NOV26a, | DVRLEYLEEVASIVLKFKPDKWSKLIGAEENVALFTEFFEKPDVQVLVLTLNAAGMII |
| CG54435-01 | |
| Protein Sequence | PCLGFPQSLKSKGVYFIKTKSENINKDNYPARLLYGDISPTPVDQLIAVVEEVLSSLL |
| | NQSENMAGWPQVVSEDIVKQVHRLKNEMFVMSGKIKGKTLLPIPEHLGSLDGTLESME |
| | RIPSSLDNLLLHAIETTIIDWSHQIRDVLSKDSAQALLDGLHPLPQVEFEFWDTRLLN |
| | LKCIHEQLNRPKVNKIVEILEKAKSCYWPALQNVYTNVTEGLKEANDIVLYLKPLRIL |
| | LEEMEQADFTMLPTFIAKVLDTICFIWATSEYYNTPARIIVILQEFCNQIIEMTRTFL |
| | SPEEVLKGLQGEIEEVLSGISLAVNVLKELYQTYDFCCVNMKLFFKDKEPVPWEFPSS |
| | LAFSRINSFFQRIQTIEELYKTAIEFLKLEKIELGGVRGNLLGSLVTRIYDEVFELVK |
| | VFADCKYDPLDPGDSNFDRDYADFEIKIQDLDRRLATIFCQCFDDCSCIKSSAKLLYM |
| | CGGLMERPLILAEVAPRYSVMLELFDAELDNAKILYDAQMAASEEGNIPLIHKNNPPV |
| | ACQLKWSLELQERLEVSMKHLKHVEHPVMSGAEAKLTYQKYDEMMELLRCHREKIYQQ |
| | WVAGVDQDCHFNLGQPLILRDAASNLIHVNFSKALVAVLREVKYLNFQQQKEIPDSAE |
| | SLFSENETFRKFVGNLELIVGWYNEIKTIVKAVEFLLIKSELEAIDVKLLSAETTLFW |
| | NGEGVFQYIQEVREILHNLQNRMQKAKQNIEGISQAMKDWSANPLFERKDNKKEALLD |
| | LDGRIANLNKRYAAVRDAGVKIQAMENAELFRADTLSLPWKDYVIYIDDMVLDEFDQF |
| | IRKSLSFLMDNMVIDESIAPLFEIRMELDEDGLTFNPTLEVGSDRGFLALIEGLVNDI |
| | YNVARLIPRLAKDRMNYKMDLEDNTDLIEMREEVSSLVINAMKEAEEYQDSFERYSYL |
| | WTDNLQEFMKNFLIYGCAVTAEDLDTWTDDTIPKTPPTLAQFQEQIDSYEKLYEEVSK |
| | CENTKVFHGWLQCDCRPFKQALLSTIRRWGFMFKRHLSNHVTNSLADLEAFMKVARMG |
| | LTKPLKEGDYDGLVEVMGHLMKVKERQAATDNNFEPLKQTIELLKTYGEEMPEEIHLK |
| | LQELPEHWANTKKLAIQVKLTVAPLQANEVSILRRKCQQFELKQHEFRERFRREAPFS |
| | FSDPNPYKSLNKQQKSISAMEGIMEALSKSGGLFEVPVPDYKQLKACHREVRLLKELW |
| | DMVVVVNTSIEDWKTTKWKDINVEQMDIDCKKFAKDMRSLDKEMKTWDAFVGLDNTVK |
| | NVITSLPAVSELQNPAIRERHWQQLMQATQVKFKMSEETTLADLLQLNLHSYEDEVRN |
| | IVDKAVKESGMEKVLKALDSTWSMNEFQHEPHPRTGTMMLKSSEVLVETLEDNQVQLQ |
| | NLMNSKYLAHFLKEVTSWQQKLSTADSVISIWFEVQRTWSHLESIFIGSEDIRTQLPG |
| | DSQRFDDTNQEFKALMEDAVKTPNVVEATSKPGLYNKLEALKKSLAICEKALAEYLET |
| | KRLAFPRFYFVSSADLLDILSNGNDPVEVSRHLSKLPDSLCKLKFRLDASDKPLKVGL |
| | GMYSKEDEYMVFDQECDLSGQVEVWLNRVLDRMCSTLRHEIPEAVVTYEEKPREQWIL |
| | DYPAQVALTCTQIWWTTEVGLAFARLEEGYENAIRDYNKKQISQLNVLITLLMGNLNA |
| | GDRMKIMTICTIDVHARDVVAKMIVAKVESSQAFTWQAQLRHRWDEEKRHCFANICDA |
| | QIQYSYEYLGNTPRLVITPLTDRCYITLTQSLHLIMGGAFAGPAGTGKTETTKDLGRA |
| | LGTMVYVFNCSEQMDYKSCCNIYKGLAQTGAWCCFDEFNRISVEVLSVIAVQVKCVQD |
| | AIRAKKKAFNFLGEIIGLIPTVGIFITMNPGYAGRAELPENLKALFRPCAMVVPDFEL |
| | ICEIMLMAEGFLEARLLARKFITLYTLCKELLSKQDHYDWGLRAIKSVLVVAGSLKRG |
| | DPSRAEDQVLMRALRDFNIPKIVTDDLPVFMGLIGDLFPALDVPRKRDLNFEKIIKQS |
| | IVELKLQAEDSFVLKVVQLEELLQVRHSVFIVGNAGSGKSQVLKSLNKTYQNLKRKPV |
| | AVDLDPKAVTCDELFGIINPVTREWKDGLFSTIMRDLANITHDGPKWIILDGDIDPMW |
| | IESLNTVMDDNKVLTLASNERIPLNRTMRLVFEISHLRTATPATVSRAGILYINTADL |
| | GWNPVVSSWIERRKVQSEKANLMILFDKYLPTCLDKLRFGFKKITPVPEITVIQTILY |
| | LLECLLTEKTVPPDSPRELYELYFVFTCFWAFGGAMFQDQLVDYRVEFSKWWINEFKT |
| | IKFPSQGTIFDYYIDPDTKKFLPWTDKVPSFELDPDVPLQASLVHTTETIRIRYFMDL |
| | LMEKSWPVMLVGNAGTGKSVLMGDKLESLNTDNYLVQAVPFNFYTTSAMLQGVLEKPL |
| | EKKSGRNYGPPGTKKLVYFIDDMNMPEVDKYGTVAPHTLIRQHMDHRHWYDRHKLTLK |
| | DIHNCQYVACMNPTSGSFTIDSRLQRHFCVFAVSFPGQEALTTIYNTILTQHLAFRSV |
| | SMAIQRISSQLVAAALALHQKITATFLPTAIKFHYVFNLRDLSNIFQGLLFSTAEVLK |
| | TPLDLVRLWLHETERVYGDKMVDEKDQETLHRVTMASTKKFFDDLGDELLFAKPNIFC |
| | HFAQGIGDPKYVPVTDMAPLNKLLVDVLDSYNEVNAVMNLVLFEDAVAHICRINRILE |
| | SPRGNALLVGVGGSGKQSLSRLAAYISGLDVFQITLKKGYGIPDLKIDLAAQYIKAAV |
| | KNVPSVFLMTDSQVAEEQFLVLIMDLLASGEIPGLFMEDEVENIISSMRPQVKSLGMN |
| | DTRETCWKFFIEKVRRQLKVILCFSPVGSVLRVRARKFPAVVNCTAIDWFHEWPEDAL |
| | VSVSARFLEETEGIPWEVKASISFFMSYVHTTVNEMSRVYLATERRYNYTTPKTFLEQ |
| | IKLYQNLLAKKRTELVAKIERLENGLMKLQSTASQVDDLKAKLAIQEAELKQKNESAD |
| | QLIQVVGIEAEKVSKEKAIADQEEVKVEVINKNVTEKQKACETDLAKAEPALLAAQEA |
| | LDTLNKNNLTELKSFGSPPDAVVNVTAAVMILTAPGGKIPKDKSWKAAKIMMGKVDTF |
| | LDSLKKFDKEHIPEACLKAFKPYQGNPTFDPEFIRSKSTAAAGLCSWCINIVRFYEVY |
| | CDVAPKRQALEEANAELAEAQEKLSRIKNKIAELNANLSNLTSAFEKATAEKIKCQQE |
| | ADATNRVILLANRLVGGLASENIRWAESVENFRSQGVTLCGDVLLISAFVSYVGYFTK |
| | KYRNELMEKFWIPYIHNLKVPIPITNGLDPLSLLTDDADVATNNNQGLPSDRMSTENA |
| | TILGNTERWPLIVDAQLQGIKWIKNKYRSELKAIRLGQKSYLDVIEQAISEGDTLLIE |
| | NIGETVDPVLDPLLGRNTIKKGKYIKIGDKEVEYHPKFRLILHTKYFNPHYKPEMQAQ |
| | CTLINTLVTRDGLEDQLLAAVVAKERPDLEQLKANLTKSQNEFKIVLKELEDSLLARL |
| | SAASGNFLGDTALVENLETTKHTASEIEEKVVEAKITEVKINEARENYRPAAERASLL |
| | YFILNDLNKINPVYQFSLKAFNVVFEKAIQRTTPANEVKQRVINLTDEITYSVYNYTA |
| | RGLFERDKLIFLAQVTFQVLSMKKELNPVELDFLLRFPFKAGVVSPVDFLQHQGWGGI |
| | KALSEMDEFKNLDSDIEGSAKRWKKLVESEAPEKEIFPKEWKNKTALQKLCMVRCLRP |
| | DRMTYAIKNFVEEKMGSKFVEGRSVEFSKSYEESSPSTSIFFILSPGVDPLKDVEALG |
| | KKLGFTIDNGKLHNVSLGQGQEVVAENALDVAAEKGHWVILQNIHLVARWLGTLDKKL |
| | ECYSTGSHEDYRVFISAEPAPSPETHIIPQGILENAIKITNEPPTGMHANLHKALDLF |
| | TQDTLEMCTKEMEFKCMLFALCYFHAVVAERRKFGAQGWNRSYPFNNGDLTISINVLY |
| | NYLEANPKVPWDDLRYLFGEIMYGGHITDDWDRRLCRTYLAEYIRTEMLEGDVLLAPG |
| | FQIPPNLDYKGYHEYIDENLPPESPYLYGLHPNAEIGFLTVTSEKLFRTVLEMQPKET |
| | DSGAGTGVSREEKVKAVLDDILEKIPETFNMAEIMAKAAEKTPYVVVAFQECERMNIL |
| | TNEMRRSLKELNLGLKGELTITTDVEDLSTALFYDTVPDTWVARAYPSMMGLAAWYAD |
| | LLLRIRELEAWTTDFALPTTVWLAGFFNPQSFLTATMQSMARKNEWPLDKMCLSVEVT |
| | KKNREDMTAPPREGSYVYGLFMEGARWDTOTGVIAEARLKELTPANPVIFIKAIPVDR |
| | METKNTYECPVYKTRIRGPTYVWTFNLKTKEKAAKWILAAVALLLQV |

Further analysis of the NOV26a protein yielded the following properties shown in Table 26B.

TABLE 26B
Protein Sequence Properties NOV26a

| | |
|---|---|
| PSort | 0.6000 probability located in plasma membrane; |
| analysis: | 0.4000 probability located in Golgi body; 0.3000 |
| | probability located in endoplasmic reticulum (membrane); |
| | 0.3000 probability located in microbody (peroxisome) |
| SignalP | No Known Signal Sequence Predicted |
| analysis: | |

A search of the NOV26a protein against the Geneseq database, a proprietary database that contains sequences published in patents and patent publication, yielded several homologous proteins shown in Table 26C.

TABLE 26C
Geneseq Results for NOV26a

| | | NOV26a | Identities/ | |
|---|---|---|---|---|
| | | Residues/ | Similarities | |
| Geneseq | Protein/Organism/Length | Match | forthe | Expect |
| Identifier | [Patent #, Date] | Residues | Matched Region | Value |
| ABB60101 | Drosophila melanogaster | 1 . . . 4454 | 2669/4492 (59%) | 0.0 |
| | polypeptide SEQ ID NO 7095 - | 19 . . . 4471 | 3378/4492 (74%) | |
| | Drosophila melanogaster, 4472 | | | |
| | aa. [WO200171042-A2, 27 SEP. | | | |
| | 2001] | | | |
| AAM78879 | Human protein SEQ ID NO 1541 - | 2314 . . . 4455 | 1504/2143 (70%) | 0.0 |
| | Homo sapiens, 2143 aa. | 1 . . . 2143 | 1804/2143 (83%) | |
| | [WO200157190-A2, 09 AUG. | | | |
| | 2001] | | | |
| AAM79863 | Human protein SEQ ID NO 3509 - | 2254 . . . 3929 | 1160/1677 (69%) | 0.0 |
| | Homo sapiens, 2127 aa. | 1 . . . 1677 | 1397/1677 (83%) | |
| | [WO200157190-A2, 09 AUG. | | | |
| | 2001] | | | |
| AAM79862 | Human protein SEQ ID NO 3508 - | 2254 . . . 3929 | 1160/1677 (69%) | 0.0 |
| | Homo sapiens, 2127 aa. | 1 . . . 1677 | 1397/1677 (83%) | |
| | [WO200157190-A2, 09 AUG. | | | |
| | 2001] | | | |
| AAU74335 | Human cytoskeleton-associated | 3279 . . . 4455 | 1173/1177 (99%) | 0.0 |
| | protein (CYSKP) #6 - Homo | 14 . . . 1190 | 1175/1177 (99%) | |
| | sapiens, 1190 aa. | | | |
| | [WO200185942-A2, 15 NOV. | | | |
| | 2001] | | | |

In a BLAST search of public sequence datbases, the NOV26a protein was found to have homology to the proteins shown in the BLASTP data in Table 26D.

TABLE 26D
Public BLASTP Results for NOV26a

| | | NOV26a | Identities/ | |
|---|---|---|---|---|
| Protein | | Residues/ | Similarities | |
| Accession | | Match | for the | Expect |
| Number | Protein/Organism/Length | Residues | Matched Portion | Value |
| P23098 | Dynein beta chain, ciliary - | 1 . . . 4455 | 3040/4467 (68%) | 0.0 |
| | Tripneustes gratilla (Hawaian sea | 6 . . . 4466 | 3658/4467 (81%) | |
| | urchin), 4466 aa. | | | |
| P39057 | Dynein beta chain, ciliary - | 1 . . . 4455 | 3039/4467 (68%) | 0.0 |
| | Anthocidaris crassispina (Sea | 6 . . . 4466 | 3657/4467 (81%) | |
| | urchin), 4466 aa. | | | |
| Q9NYC9 | Ciliary dynein heavy chain 9 | 1 . . . 4455 | 2812/4469 (62%) | 0.0 |
| | (Axonemal beta dynein heavy | 22 . . . 4486 | 3518/4469 (77%) | |
| | chain 9) - Homo sapiens | | | |
| | (Human), 4486 aa. | | | |
| AAF55834 | CG3723-PA - Drosophila | 1 . . . 4454 | 2683/4482 (59%) | 0.0 |
| | melanogaster (Fruit fly), 4496 aa. | 19 . . . 4495 | 3400/4482 (74%) | |
| Q9VDG0 | DHC93AB protein - Drosophila | 1 . . . 4454 | 2669/4492 (59%) | 0.0 |
| | melanogaster (Fruit fly), 4472 aa. | 19 . . . 4471 | 3378/4492 (74%) | |

PFam analysis predicts that the NOV26a protein contains the domains shown in the Table 26E.

TABLE 26E
Domain Analysis of NOV26a

| | | Identities/ | |
|---|---|---|---|
| | NOV26a | Similarities | |
| | Match | for the | Expect |
| Pfam Domain | Region | Matched Region | Value |
| Luteo_ORF3 | 1022 . . . 1055 | 9/35 (26%) | 0.41 |
| | | 21/35 (60%) | |
| Dynein_heavy | 3751 . . . 4454 | 434/777 (56%) | 0 |
| | | 674/777 (87%) | |

### Example 27

The NOV27 clone was analyzed, and the nucleotide and encoded polypeptide sequences are shown in Table 27A.

TABLE 27A
NOV27 Sequence Analysis

| | SEQ ID NO:305 | 2675 bp | |
|---|---|---|---|
| NOV27a, | CTGTCTGTGGTGTGGCTGTGGGACCCGTGAGCAAGCAGCGACGCCAGCGGCGGAGAAC | | |
| CG154465-01 | | | |
| DNA Sequence | CGACGAAAGGTGTCACCACAGTGATGGCAGTGGAGGACAGCACGCTGCAAGTAGTGGT | | |
| | ACGGGTGCGGCCCCCCACCCCTCGGGAGCTGGACAGTCAGCGGCGGCCAGTGGTTCAG | | |
| | GTGGTGGACGAGCGGGTGCTGGTGTTTAACCCTGAGGAGCCCGATGGAGGGTTCCCTG | | |
| | GCCTGAAATGGGGTGGCACCCATGATGGCCCCAAGAAGAAGGGCAAAGACCTGACGTT | | |
| | TGTCTTTGACCGGGTCTTTGGCGAGGCGGCCACCCAACAGGACGTGTTCCAGCACACC | | |
| | ACGCACAGCGTCCTGGACAGCTTCCTCCAGGGCTACAACTGCTCAGTGTTTGCCTACG | | |
| | GGGCCACCGGGGCTGGGAAGACACACACCATGCTGGGAAGGGAGGGGGACCCCGGCAT | | |
| | CATGTACCTGACCACCGTGGAACTGTACAGGCGCCTGGAGGCCCGCCAGCAGGAGAAG | | |
| | CACTTCGAGGTGCTCATCAGCTACCAGGAGGTCTATAATGAACAGATCCATGACCTCC | | |
| | TGGAGCCCAAGGGGCCCCTTGCCATCCGCGAGGACCCCGACAAGGGGGTGGTGGTGCA | | |
| | AGGACTTTCTTTCCACCAGCCAGCCTCAGCCGAGCAGCTGCTGGAGATACTGACCAGG | | |
| | GGGAACCGTAACCGCACGCAGCACCCCACTGATGCCAACGCGACTTCCTCCCGCTCCC | | |
| | ATGCCATCTTCCAGATCTTTGTGAAGCAGCAGGACCGGGTTCCAGGACTGACCCAGGC | | |
| | TGTCCAGGTGGCCAAGATGAGCCTGATTGACCTGGCTGGCTCAGAGCGGGCATCCAGC | | |
| | ACCCATGCGAAGGGGGAGCGGCTGCGGGAGGGGGCCAACATCAACCGCTCTCTCCTGG | | |
| | CGCTCATCAACGTCCTCAATGCCTTGGCCGATGCAAAGGTAGGCCGCAAGACCCATGT | | |
| | GCCCTACCGGGACAGCAAACTGACCCGCCTGCTCAAAGACTCCCTCGGGGGCAACTGC | | |
| | CGCACAGTGATGATCGCTGCCATCAGCCCCTCCAGCCTGACCTACGAGGACACGTACA | | |
| | ACACCCTCAAATATGCCGACCGCGCCAAGGAGATCAGGCTCTCGCTGAAGAGCAATGT | | |
| | GACCAGCCTGGACTGTCACATCAGCCAGTATGCTACCATCTGCCAACAGCTCCAGGCT | | |
| | GAGGTAGCCGCTCTGAGGAAGAAGCTCCAAGTGTATGAGGGGGGAGGCCAGCCCCCAC | | |
| | CACAGGACCTCCCAGGATCTCCCAAGTCGGGACCACCACCAGAACACCTTCCCAGCTC | | |
| | CCCCTTGCCACCCCACCCTCCCAGCCAGCCCTGCACCCCAGAGCTCCCTGCAGGGCCT | | |
| | AGAGCCCTTCAAGAGGAGAGTCTGGGGATGGAGGCCCAGGTGGAGAGGGCCATGGAAG | | |
| | GGAACTCTTCAGACCAGGAGCAGTCCCCAGAGGATGAGGATGAAGGCCCACCTGAGGA | | |
| | GGTTCCAACCCAGATGCCAGAGCAGAACCCCACACATGCACTGCCAGAGTCCCCTCGC | | |
| | CTGACCCTGCAGCCCAAGCCAGTCGTGGGCCACTTCTCAGCACGGGAACTGGATGGGG | | |
| | ACCGTTCTAAGCAGTTGGCCCTAAAGGTGCTGTGCGTTGCCCAGCGGCAGTACTCCCT | | |
| | GCTCCAAGCAGCCAACCTCCTGACGCCCGACATGATCACAGAGTTTGAGACCCTACAG | | |
| | CAGCTGGTGCAAGAGGAAAAAATTGAGCCTGGGGCAGAGGCCTTGAGGACTTCAGGCC | | |
| | TGGCCAGGGGGGCACCTCTGGCTCAGGAGCTGTGTTCAGAGTCAATCCCTGTGCCGTC | | |
| | TCCTCTCTGCCCAGAGCCTCCAGGATACACTGGCCCTGTGACCCGGACTATGGCGAGG | | |
| | CGACTGAGTGGCCCCCTGCACACCCTGGGAATCCCGCCTGGACCCAACTGCACCCCAG | | |
| | CCCAGGGGTCCCCATGGCCCATGGAGAAGAAGAGGAGGAGACCAAGCGCCTTGGAGGC | | |
| | AGACAGTCCCATGGCCCCAAAGCGGGGCACCAAGCGCCAGCGCCAGTCCTTCCTGCCC | | |
| | TGCCTAAGGAGAGGGTCTCTGCCTGACACCCAACCTTCACAGGGGCCCAGCACCCCCA | | |
| | AAGGAGAAAGGGCCTCCTCCCCCTGCCATTCCCCTCGCGTTTGCCCAGCCACAGTCAT | | |
| | CAAAAGCCGGGTGCCCCTGGGCCCTTCCGCCATGCAGAACTGCTCCACCCCGCTGGCT | | |
| | CTGCCCACTCGAGACCTCAATGCCACCTTTGATCTCTCTGAGGAGCCTCCCTCAAAGC | | |
| | CCAGTTTCCATGAATGCATTGGCTGGGACAAAATACCCCAGGAGCTGAGCAGGCTGGA | | |
| | CCAGCCCTTCATCCCCAGGGCACCTGTGCCCCTGTTCACCATGAAGGGCCCCAAGCCA | | |
| | ACATCTTCCCTCCCTGGGACCTCTGCCTCCAAGAAGAAGCGCGTTGCGAGTTCCTCAG | | |
| | TCTCCCATGGCCGCAGCCGCATCGCCCGCCTCCCCAGCAGCACTTTGAAGAGGCCAGC | | |
| | TGGGCCCCTTGTACTCCCAGGTGACTGGCACTAGGGACACGGATAGCCTGGGCCATGG | | |
| | AGGCCGATGAAGACAAGAAGGAGGAGGGGACGGGGAGCTGAGACCCAGAAGAAAGGAG | | |
| | GGCCTAG | | |
| | ORF Start: ATG at 82 | | ORF Stop: TAG at 2584 |
| | SEQ ID NO:306 | 834 aa | MW at 91153.5 kD |

| | |
|---|---|
| NOV27a, | MAVEDSTLQVVVRVRPPTPRELDSQRRPVVQVVDERVLVFNPEEPDGGFPGLKWGGTH |
| CG154465-01 | |
| Protein Sequence | DGPKKKGKDLTFVFDRVFGEAATQQDVPQHTTHSVLDSFLQGYNCSVFAYGATGAGKT |
| | HTMLCREGDPGIMYLTTVELYRRLEARQQEKHFEVLISYQEVYNEQIHDLLEPKGPLA |
| | IREDPDKGVVVQGLSFHQPASAEQLLEILTRGNRNRTQHPTDANATSSRSHAIFQIFV |
| | KQQDRVPGLTQAVQVAKMSLIDLAGSERASSTHAKGERLREGANINRSLLALINVLNA |
| | LADAKVGRKTHVPYRDSKLTRLLKDSLGGNCRTVMIAAISPSSLTYEDTYNTLKYADR |
| | AKEIRLSLKSNVTSLDCHISQYATICQQLQAEVAALRKKLQVYEGGGQPPPQDLPGSP |
| | KSGPPPEHLPSSPLPPHPPSQPCTPELPAGPRALQEESLGMEAQVERAMEGNSSDQEQ |
| | SPEDEDEGPAEEVPTQMPEQNPTHALPESPRLTLQPKPVVGHFSARELDGDRSKQLAL |
| | KVLCVAQRQYSLLQAANLLTPDMITEFETLQQLVQEEKIEPGAEALRTSGLARGAPLA |
| | QELCSESIPVPSPLCPEPPGYTGPVTRTMARRLSGPLHTLGIPPGPNCTPAQGSRWPM |
| | EKKRRRPSALEADSPMAPKRGTKRQRQSFLPCLRRGSLPDTQPSQGPSTPKGERASSP |
| | CHSPRVCPATVIKSRVPLGPSANQNCSTPLALPTRDLNATFDLSEEPPSKPSFHECIG |
| | WDKIPQELSRLDQPFIPRAPVPLFTMKGPKPTSSLPGTSACKKKRVASSSVSHGRSRI |
| | ARLPSSTLKRPAGPLVLPGDWH |

Further analysis of the NOV27a protein yielded the following properties shown in Table 27B.

TABLE 27B
Protein Sequence Properties NOV27a

| | |
|---|---|
| PSort | 0.7000 probability located in nucleus; 0.4267 |
| analysis: | probability located in mitochondrial matrix space; |
| | 0.3000 probability located in microbody (peroxisome); |
| | 0.1042 probability located in mitochondrial inner |
| | membrane |
| SignalP | No Known Signal Sequence Predicted |
| analysis: | |

A search of the NOV27a protein against the Geneseq database, a proprietary database that contains sequences published in patents and patent publication, yielded several homologous proteins shown in Table 27C.

TABLE 27C
Geneseq Results for NOV27a

| | | NOV27a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| ABB07410 | Human kinesin motor protein, | 1 . . . 830 | 828/830 (99%) | 0.0 |
| | HsKip3A - Homo sapiens, 864 aa. | 1 . . . 829 | 828/830 (99%) | |
| | [WO200196593-A2, 20 DEC. 2001] | | | |
| ABB07412 | Amino acid sequence of Kip3A | 1 . . . 360 | 354/360 (98%) | 0.0 |
| | fragment used in ATPase assay - | 1 . . . 359 | 355/360 (98%) | |
| | Homo sapiens, 383 aa. | | | |
| | [WO200196593-A2, 20 DEC. 2001] | | | |
| ABB07411 | Human HsKip3A motor domain | 5 . . . 343 | 338/339 (99%) | 0.0 |
| | fragment - Homo sapiens, 338 aa. | 1 . . . 338 | 338/339 (99%) | |
| | [WO200196593-A2, 20 DEC. 2001] | | | |
| AAU76967 | Novel human kinesin motor protein, | 8 . . . 392 | 231/391 (59%) | e−130 |
| | HsKip3d insertion mutant - Homo | 12 . . . 402 | 298/391 (76%) | |
| | sapiens, 905 aa. [WO200212268-A1, | | | |
| | 14 FEB. 2002] | | | |
| AAU76957 | Novel human kinesin motor protein, | 8 . . . 392 | 231/385 (60%) | e−130 |
| | HsKip3d - Homo sapiens, 898 aa. | 12 . . . 395 | 297/385 (77%) | |
| | [WO200212268-A1, 14 FEB. 2002] | | | |

In a BLAST search of public sequence datbases, the NOV27a protein was found to have homology to the proteins shown in the BLASTP data in Table 27D.

TABLE 27D
Public BLASTP Results for NOV27a

| | | NOV27a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q91WD7 | Similar to hypothetical protein | 8 . . . 392 | 233/385 (60%) | e−131 |
| | DKFZp434G2226 - Mus | 12 . . . 395 | 296/385 (76%) | |
| | musculus (Mouse), 886 aa. | | | |
| BAB93508 | OK/SW-CL.108 - Homo sapiens | 8 . . . 392 | 231/385 (60%) | e−129 |
| | (Human), 898 aa. | 12 . . . 395 | 297/385 (77%) | |
| Q9H0F3 | Hypothetical 102.3 kDa protein - | 8 . . . 392 | 231/385 (60%) | e−129 |
| | Homo sapiens (Human), 898 aa. | 12 . . . 395 | 297/385 (77%) | |
| Q9VSW5 | KLP67A protein (RE52076p) - | 4 . . . 452 | 213/451 (47%) | 3e−99 |
| | Drosophila melanogaster (Fruit | 5 . . . 434 | 283/451 (62%) | |
| | fly), 814 aa. | | | |
| P91945 | Kinesin like protein 67A - | 4 . . . 452 | 213/451 (47%) | 3e−99 |
| | Drosophila melanogaster (Fruit | 5 . . . 434 | 283/451 (62%) | |
| | fly), 814 aa. | | | |

PFam analysis predicts that the NOV27a protein contains the domains shown in the Table 27E.

TABLE 27E
Domain Analysis of NOV27a

| | | Identities/ | |
|---|---|---|---|
| | NOV27a | Similarities | |
| | Match | for the | Expect |
| Pfam Domain | Region | Matched Region | Value |
| kinesin | 13 . . . 388 | 158/435 (36%) | 2.3e−114 |
| | | 281/435 (65%) | |

### Example 28

The NOV28 clone was analyzed, and the nucleotide and encoded polypeptide sequences are shown in Table 28A.

TABLE 28A
NOV28 Sequence Analysis

| | SEQ ID NO:307 | 1872 bp | |
|---|---|---|---|
| NOV28a, | CGCGGCGGCTGGCGTCGGGAAAGTACAGTAAAAAGTCCGAGTGCAGCCGCCGGGCGCA | | |
| CG154492-01 | | | |
| DNA Sequence | GGATGGGATCCGGCTCCTCCAGCTACCGGCCCAAGGCCATCTACCTGGACATCGATGG | | |
| | ACGCATTCAGAAGGTAATCTTCAGCAAGTACTGCAACTCCAGCGACATCATGGACCTG | | |
| | TTCTGCATCGCCACCGGCCTGCCTCGGAACACGACCATCTCCCTGCTGACCACCGACG | | |
| | ACGCCATGGTCTCCATCGACCCCACCATGCCCGCGAATTCAGAACGCACTCCGTACAA | | |
| | AGTGAGACCTGTGGCCATCAAGCAACTCTCCGCTGGTGTCGAGGACAAGAGAACCACA | | |
| | AGCCGTGGCCAGTCTGCTGAGAGACCACTCAGGGACAGACGGGTTGTGGGCCTGGAGC | | |
| | AGCCCCGGAGGGAAGGAGCATTTGAAAGTGGACAGGTAGAGCCCAGGCCCAGAGAGCC | | |
| | CCAGGGCTGCTACCAGGAAGGCCAGCGCATCCCTCCACAGAGAGAAGAATTAATCCAG | | |
| | AGCGTGCTGGCGCAGGTTGCAGAGCAGTTCTCAAGAGCATTCAAAATCAATGAACTGA | | |
| | AAGCTGAAGTTGCAAATCACTTGGCTGTCCTAGAGAAACGCGTGGAATTGGAAGGACT | | |
| | AAAAGTGGTGGAGATTGAGAAATGCAAGAGTGACATTAAGAAGATGAGGGAGGAGCTG | | |
| | GCGGCCAGAAGCAGCAGGACCAACTGCCCCTGTAAGTACAGTTTTTTGGATAACCACA | | |
| | AGAAGTTGACTCCTCGACGCGATGTTCCCACTTACCCCAAGTACCTGCTCTCTCCAGA | | |
| | GACCATCGAGGCCCTGCGGAAGCCGACCTTTGACGTCTGGCTTTGGGAGCCCAATGAG | | |
| | ATGCTGAGCTGCCTGGAGCACATGTACCACGACCTCGGGCTGGTCAGGGACTTCAGCA | | |
| | TCAACCCTGTCACCCTCAGGAGGTGGCTGTTCTGTGTCCACGACAACTACAGAAACAA | | |
| | CCCCTTCCACAACTTCCGGCACTGCTTCTGCGTGGCCCAGATGATGTACAGCATGGTC | | |
| | TGGCTCTGCAGTCTCCAGGAGAAGTTCTCACAAACGGATATCCTGATCCTAATGACAG | | |
| | CGGCCATCTGCCACGATCTGGACCATCCCGGCTACAACAACACGTACCAGATCAATGC | | |
| | CCGCACAGAGCTGGCGGTCCGCTACAATGACATCTCACCGCTGGAGAACCACCACTGC | | |
| | GCCGTGGCCTTCCAGATCCTCGCCGAGCCTGAGTGCAACATCTTCTCCAACATCCCAC | | |
| | CTGATGGGTTCAAGCAGATCCGACAGGGAATGATCACATTAATCTTGGCCACTGACAT | | |
| | GGCAAGACATGCAGAAATTATGGATTCTTTCAAAGAGAAAATGGAGAATTTTGACTAC | | |
| | AGCAACGAGGAGCACATGACCCTCAGCGACCGTGAGAAGTCAGAAGGCCTTCCTGTGG | | |
| | CACCGTTCATGGACCGAGACAAAGTGACCAAGGCCACAGCCCAGATTGGGTTCATCAA | | |
| | GTTTGTCCTGATCCCAATGTTTGAAACAGTGACCAAGCTCTTCCCCATGGTTGAGGAG | | |
| | ATCATGCTGCAGCCACTTTGGGAATCCCGAGATCGCTACGAGGAGCTGAAGCGGATAG | | |
| | ATGACGCCATGAAAGAGTTACAGAAGAAGACTGACAGCTTGACGTCTGGGGCCACCGA | | |
| | GAAGTCCAGAGAGAGAAGCAGAGATGTGAAAAACAGTGAAGGAGACTGTGCCTGAGGA | | |
| | AAGCGGGGGGCGTGGCTGCAGTTCTGGACGGGCTGGCCGAGCTGCGCGGGATCCTTGT | | |
| | GCAGGGAAGAGCTGCCCTGGGCACCTGGCACCACAAGACCATGTTTTCTAAGAACCAT | | |
| | TTTGTTCACTGATACA | | |
| | ORF Start: ATG at 61 | | ORF Stop: TGA at 1735 |
| | SEQ ID NO:308 | 558 aa | MW at 64319.9 kD |
| NOV28a, | MGSGSSSYRPKAIYLDIDGRIQKVIFSKYCNSSDIMDLFCIATGLPRNTTISLLTTDD | | |
| CG154492-01 | | | |
| Protein Sequence | AMVSIDPTMPANSERTPYKVRPVAIKQLSAGVEDKRTTSRGQSAERPLRDRRVVGLEQ | | |
| | PRREGAFESGQVEPRPREPQGCYQEGQRIPPEREELIQSVLAQVAEQFSRAFKINELK | | |
| | AEVANHLAVLEKRVELEGLKVVEIEKCKSDIKKMREELAARSSRTNCPCKYSFLDNHK | | |
| | KLTPRRDVPTYPKYLLSPETIEALRKPTFDVWLWEPNEMLSCLEHMYHDLGLVRDFSI | | |
| | NPVTLRRWLFCVHDNYRNNPFHNFRHCFCVAQMMYSMVWLCSLQEKFSQTDILILMTA | | |
| | AICHDLDHPGYNNTYQINARTELAVRYNDISPLENHHCAVAFQILAEPECNIFSNIPP | | |
| | DGFKQIRQGMITLILATDMARHAEIMDSFKEKMENFDYSNEEHMTLSDREKSEGLPVA | | |
| | PFMDRDKVTKATAQIGFIKFVLIPMFETVTKLFPMVEEIMLQPLWESRDRYEELKRID | | |
| | DAMKELQKKTDSLTSGATEKSRERSRDVKNSEGDCA | | |
| | SEQ ID NO:309 | 1653 bp | |
| NOV28b, | CGGGAAAGTACAGTAAAAAGTCCGAGTGCAGCCACCGGGCGCAGGATGGCGTCCGGCT | | |
| CG154492-02 | | | |
| DNA Sequence | CCTCCGGCTACCGGCCCAAGGCCATCTACCTGGACATCGATGGACGCATTCAGAAGGT | | |
| | AATCTTCAGCAAGTACTGCAACTCCAGCGACATCATGGACCTGTTCTGCATCGCCACC | | |
| | GGCCTGCCTCGGAACACGACCATCTCCCTGCTGACCACCGACGACGCCATGGTCTCCA | | |
| | TCGACCCCACCATGCCCGCGAATTCAGAACGCACTCCGTACAAAGTGAGACCTGTGGC | | |
| | CATCAAGCAACTCTCCGAGAGAGAAGAATTAATCCAGAGCGTGCTGGCGCAGGTTGCA | | |
| | GAGCAGTTCTCAAGAGCATTCAAAATCAATGAACTGAAAGCTGAAGTTGCAAATCACT | | |
| | TGGCTGTCCTACACAAACGCGTGGAATTGGAAGGACTAAAAGTGGTGGAGATTGAGAA | | |
| | ATGCAAGAGTGACATTAAGAAGATGAGGGAGGAGCTGGCGGCCAGAAGCAGCAGGACC | | |
| | AACTGCCCCTGTAAGTACAGTTTTTTGGATAACCACAAGAAGTTGACTCCTCGACGCG | | |
| | ATGTTCCCACTTACCCCAAGTACCTGCTCTCTCCAGAGACCATCGAGGCCCTGCGGAA | | |
| | GCCGACCTTTGACGTCTGGCTTTGGGAGCCCAATGAGATGCTGAGCTGCCTGGAGCAC | | |
| | ATGTACCACGACCTCGGGCTGGTCAGGGACTTCAGCATCAACCCTGTCACCCTCAGGA | | |
| | GGTGGCTGTTCTGTGTCCACGACAACTACAGAAACAACCCCTTCCACAACTTCCGGCA | | |
| | CTGCTTCTGCGTGGCCCAGATGATGTACAGCATGGTCTGGCTCTGCAGTCTCCAGGAG | | |
| | AAGTTCTCACAAACGGATATCCTGATCCTAATGACAGCGGCCATCTGCCACGATCTGG | | |
| | ACCATCCCGGCTACAACAACACGTACCAGATCAATGCCCGCACAGAGCTGGCGGTCCG | | |
| | CTACAATGACATCTCACCGCTGGAGAACCACCACTGCGCCGTGGCCTTCCAGATCCTC | | |
| | GCCGAGCCTGAGTGCAACATCTTCTCCAACATCCCACCTGATGGGTTCAAGCAGATCC | | |
| | GACAGGGAATCATCACATTAATCTTGGCCACTGACATGGCAAGACATGCAGAAATTAT | | |
| | GGATTCTTTCAAAGGGAAAATGGAGAATTTTGACTACAGCAACGAGGAGCACATGACC | | |
| | CTGCTGAAGATGATTTTGATAAAATGCTGTGATATCTCTAACGAGGTCCGTCCAATGG | | |
| | AAGTCGCAGAGCCTTGGGTGGACTGTTTATTAGAGGAATATTTTATGCAGAGCGACCG | | |
| | TGAGAAGTCAGAAGGCCTTCCTGTGGCACCGTTCATGGACCGAGACAAAGTGACCAAG | | |
| | GCCACAGCCCAGATTGGGTTCATCAAGTTTGTCCTGATCCCAATGTTTGAAACAGTGA | | |
| | CCAAGCTCTTCCCCATGGTTGAGGAGATCATGCTGCAGCCACTTTGGGAATCCCGAGA | | |
| | TCGCTACGAGGAGCTGAAGCGGATAGATGACGCCATGAAAGAGTTACAGAAGAAGACT | | |
| | GACAGCTTGACGTCTGGCGCCACCGAGAAGTCCAGAGAGAGAAGCAGAGATGTGAAAA | | |
| | ACAGTGAAGGAGACTGTGCCTGAGGAAAG | | |
| | ORF Start: ATG at 46 | | ORF Stop: TGA at 1645 |
| | SEQ ID NO:310 | 533 aa | MW at 61606.3 kD |

| | |
|---|---|
| NOV28b, | MGSGSSGYRPKAIYLDIDGRIQKVIFSKYCNSSDIMDLFCIATGLPRNTTISLLTTDD |
| CG154492-02 | |
| Protein Sequence | ANVSIDPTMPANSERTPYKVRPVAIKQLSEREELIQSVLAQVAEQFSRAFKINELKAE |
| | VANHLAVLEKRVELEGLKVVEIEKCKSDIKKMREELAARSSRTNCPCKYSFLDNHKKL |
| | TPRRDVPTYPKYLLSPETIEALRKPTFDVWLWEPNEMLSCLEHMYHDLGLVRDFSINP |
| | VTLRRWLFCVHDNYRNNPFHNFRHCFCVAQMMYSMVWLCSLQEKFSQTDILILMTAAI |
| | CHDLDHPCYNNTYQINARTELAVRYNDISPLENHHCAVAFQILAEPECNIFSNIPPDG |
| | FKQIRQGMITLILATDMARHAEIMDSFKGKMENFDYSNEEHMTLLKMILIKCCDISNE |
| | VRPMEVAEPWVDCLLEEYFMQSDREKSEGLPVAPFMDRDKVTKATAQIGFIKFVLIPM |
| | FETVTKLFPMVEEIMLQPLWESRDRYEELKRIDDAMKELQKKTDSLTSGATEKSRERS |
| | RDVKNSEGDCA |

Sequence comparison of the above protein sequences yields the following sequence relationships shown in Table 28B.

TABLE 28B
Comparison of NOV28a against NOV28b.

| | | Identities/ |
|---|---|---|
| | | Similarities for |
| Protein | NOV28a Residues/ | the Matched |
| Sequence | Match Residues | Region |
| NOV28b | 1 . . . 558 | 461/593 (77%) |
| | 1 . . . 533 | 470/593 (78%) |

Further analysis of the NOV28a protein yielded the following properties shown in Table 28C.

TABLE 28C
Protein Sequence Properties NOV28a

| | |
|---|---|
| PSort | 0.7600 probability located in nucleus; 0.1000 probability |
| analysis: | located in mitochondrial matrix space; 0.1000 probability |
| | located in lysosome (lumen); 0.1000 probability located |
| | in plasma membrane |
| SignalP | No Known Signal Sequence Predicted |
| analysis: | |

A search of the NOV28a protein against the Geneseq database, a proprietary database that contains sequences published in patents and patent publication, yielded several homologous proteins shown in Table 28D.

TABLE 28D
Geneseq Results for NOV28a

| | | | Identities/ | |
|---|---|---|---|---|
| | | | Similarities for | |
| Geneseq | Protein/Organism/Length | NOV28a Residues/ | the Matched | Expect |
| Identifier | [Patent #, Date] | Match Residues | Region | Value |
| ABG61846 | Prostate cancer-associated protein | 1 . . . 558 | 558/593 (94%) | 0.0 |
| | #47 - Mammalia, 593 aa. | 1 . . . 593 | 558/593 (94%) | |
| | [WO200230268-A2, 18 APR. 2002] | | | |
| AAY28561 | Cyclic-GMP specific phosphodiesterase | 1 . . . 558 | 558/593 (94%) | 0.0 |
| | (PDE9A) - Homo sapiens, 593 aa. | 1 . . . 593 | 558/593 (94%) | |
| | [WO9929873-A1, 17 JUN. 1999] | | | |
| AAY39285 | Phosphodiesterase 10 (PDE10) clone | 14 . . . 558 | 544/580 (93%) | 0.0 |
| | FB68.2 - Homo sapiens, 580 aa. | 1 . . . 580 | 544/580 (93%) | |
| | [WO9942596-A2, 26 AUG. 1999] | | | |
| AAY39284 | Phosphodiesterase 10 (PDE10) clone | 1 . . . 558 | 463/593 (78%) | 0.0 |
| | FB76.2 - Homo sapiens, 533 aa. | 1 . . . 533 | 472/593 (79%) | |
| | [WO9942596-A2, 26 AUG. 1999] | | | |
| AAB92673 | Human protein sequence SEQ ID NO: | 148 . . . 558 | 411/446 (92%) | 0.0 |
| | 11043 - Homo sapiens, 474 aa. | 29 . . . 474 | 411/446 (92%) | |
| | [EP1074617-A2, 07 FEB. 2001] | | | |

In a BLAST search of public sequence datbases, the NOV28a protein was found to have homology to the proteins shown in the BLASTP data in Table 28E.

TABLE 28E
Public BLASTP Results for NOV28a

| | | | Identities/ | |
|---|---|---|---|---|
| Protein | | | Similarities for | |
| Accession | | NOV28a Residues/ | the Matched | Expect |
| Number | Protein/Organism/Length | Match Residues | Portion | Value |
| O76083 | High-affinity cGMP-specific 3′,5′- | 1 . . . 558 | 558/593 (94%) | 0.0 |
| | cyclic phosphodiesterase 9A | 1 . . . 593 | 558/593 (94%) | |
| | (EC 3.1.4.17) - Homo sapiens | | | |
| | (Human), 593 aa. | | | |
| AAH09047 | Similar to phosphodiesterase 9A - | 1 . . . 558 | 463/593 (78%) | 0.0 |
| | Homo sapiens (Human), 533 aa. | 1 . . . 533 | 472/593 (79%) | |
| O70628 | High-affinity cGMP-specific 3′,5′- | 1 . . . 555 | 423/590 (71%) | 0.0 |
| | cyclic phosphodiesterase 9A | 1 . . . 529 | 456/590 (76%) | |
| | (EC 3.1.4.17) - Mus musculus | | | |
| | (Mouse), 534 aa. | | | |
| Q8QZV1 | cGMP phosphodiesterase - Rattus | 1 . . . 554 | 420/589 (71%) | 0.0 |
| | norvegicus (Rat), 534 aa. | 1 . . . 528 | 457/589 (77%) | |
| AAF48205 | CG32648-PA - Drosophila | 249 . . . 549 | 152/336 (45%) | 4e−78 |
| | melanogaster (Fruit fly), 963 aa. | 48 . . . 380 | 199/336 (58%) | |

PFam analysis predicts that the NOV28a protein contains the domains shown in the Table 28F.

TABLE 28F
Domain Analysis of NOV28a

| | | Identities/ | |
|---|---|---|---|
| | | Similarities for | |
| Pfam | NOV28a | the Matched | Expect |
| Domain | Match Region | Region | Value |
| PDEase | 311 . . . 440 | 55/133 (41%) | 9.8e−52 |
| | | 90/133 (68%) | |
| PDEase | 454 . . . 498 | 14/47 (30%) | 1.1e−08 |
| | | 33/47 (70%) | |

### Example 29

The NOV29 clone was analyzed, and the nucleotide and encoded polypeptide sequences are shown in Table 29A.

TABLE 29A
NOV29 Sequence Analysis

| | SEQ ID NO:311 | 13332 bp | |
|---|---|---|---|
| NOV29a, | CTCCGGACTGGTTTCTTCTTCCTTCCCCCTTCCCCCAACTTCCCTCCACCCCTTCCAA | | |
| CG154509-01 | | | |
| DNA Sequence | TCATGCCGAACGGGACTGCGGACGTTCGGAAGCTCTTCATCTTCACTACTACCCAGAA | | |
| | TTACTTCGGGTTGATGTCTGAACTCTGGGATCAGCCACTGTTGTGCAACTGTCTTGAA | | |
| | ATCAACAACTTCTTGGATGACGGCAACCAGATGCTCCTCAGGGTGCAGCGATCCGACG | | |
| | CAGGAATCTCCTTTTCCAACACGATTGAGTTTGGTGACACAAAAGATAAAGTGCTGGT | | |
| | GTTTTTCAAGCTGCGACCTGAAGTAATTACTGATGAGAATCTACATGATAACATTCTT | | |
| | GTTTCATCTATGTTAGAGTCACCTATTAGTTCTCTTTACCAAGCAGTACGGCAAGTAT | | |
| | TCGCACCAATGTTGTTAAAGGATCAGGAATGGAGCAGAAACTTTGATCCCAAACTTCA | | |
| | GAATCTTTTGAGTGAACTAGAAGCTGGGTTGGGTATAGTTCTACGAAGATCAGACACT | | |
| | AACTTAACAAAATTGAAATTTAAGGAAGATGACACACGAGGTATCCTTACACCAAGCG | | |
| | ATGAGTTCCAGTTTTGGATAGAACAAGCTCACCGTGGAAATAAACAGATTAGTAAAGA | | |
| | AAGAGCCAATTATTTTAAAGAATTATTTGAAACAATTGCAACAGAGTTTTATAACTTG | | |
| | GACAGTCTATCCTTACTAGAAGTTGTTGACTTGGTGGAGACTACTCAGGATGTTGTAG | | |
| | ATGATGTGTGGAGACAAACAGAACATGATCATTATCCTGAGTCACGAATGTTGCATCT | | |
| | CTTAGACATCATAGGTCGTTCATTTGGAAGGTTTGTTCAGAAAAAGTTGGGAACTTTG | | |
| | AACCTGTGGGAAGATCCTTATTATCTTGTGAAAGAAAGTCTGAAAGCTGGTATTTCAA | | |
| | TTTGTGAACAGTGGGTGATAGTCTGTAATCATCTAACAGGTCAGGTGTGGCAGCGCTA | | |
| | TGTTCCTCATCCATGGAAAAATGAAAAATATTTTCCAGAAACACTTGACAAACTTGGC | | |
| | AAACGCCTTGAAGAGGTCTTGGCTATTAGAACAATTCATGAGAAGTTTCTCTATTTTC | | |
| | TACCTGCCAGTGAAGAGAAAATCATATGCCTCACTCGAGTATTTGAACCTTTTACTGG | | |
| | CCTGAATCCTGTGCAATATAATCCATATACTGAGCCCTTGTGGAAAGCTGCGGTGTCT | | |
| | CAATATGAAAAGATTATTGCACCTGCGGAACAAAAAATAGCAGGAAAATTGAAAAATT | | |
| | ATATTTCAGAAATTCAAGACAGTCCACAGCAGCTTCTTCAAGCATTCCTGAAATATAA | | |
| | AGAGTTGGTAAAGCGTCCAACTATAAGCAAAGAATTGATGTTAGAAAGAGAAACTTTA | | |
| | CTGGCAAGACTTGTGGACTCAATTAAAGATTTTCGATTAGACTTTGAGAATCGGTGCC | | |
| | GAGGAATTCCTGGTGATGCATCTGGACCACTTTCTGGCAAAAATCTTTCAGAAGTTGT | | |
| | CAACAGTATAGTTTGGGTTCGCCAGTTGGAATTGAAGGTAGATGATACTATCAAGACT | | |
| | GCAGAGGCTCTTTTATCTGACTTGCCAGGATTTCGATGTTTCCATCAAAGTGCCAAAG | | |
| | ATCTCTTAGACCAGCTTAAACTATATGAACAGGAACAATTTGATGATTGGTCCAGGGA | | |
| | TATTCAATCAGGTTTATCTGATTCCAGATCTGCTTTGTGTATTGAGGCTAGTAGTCGA | | |
| | ATTATGGAATTGGATTCTAATGATGGATTACTAAAAGTGCATTATTCAGATCGTTTGG | | |
| | TGATTCTTCTGAGAGAAGTTCGTCAGCTCTCTGCACTTGGCTTTGTTATTCCTGCCAA | | |
| | AATACAGCAAGTTGCAAACATTGCACAGAAATTCTGCAAGCAAGCAATTATTCTTAAA | | |
| | CAAGTCGCACATTTTTATAATTCTATTGATCAACAAATGATTCAAAGTCAGAGGCCAA | | |
| | TGATGTTACAATCTGCCTTAGCATTTGAACAGATAATTAAGAATTCAAAAGCAGGAAG | | |
| | TGGAGGGAAATCACAGATAACTTGGGATAATCCTAAAGAATTAGAAGGCTATATCCAA | | |
| | AAACTCCAAAATGCTGCTGAACGGCTTGCCACTGAAAATAGAAAACTGAGAAAATGGC | | |
| | ACACTACATTTTGTGAAAAGGTGGTTGTTCTTATGAATATTGATCTGCTTCGGCAGCA | | |
| | ACAGCGCTGGAAAGATGGATTACAAGAATTGAGAACTGGCTTAGCAACTGTAGAAGCA | | |
| | CAGGGATTCCAAGCAAGTGACATGCATGCATGGAAACAACACTGGAATCATCAACTGT | | |
| | ACAAAGCTCTGGAGCATCAGTACCAGATGGGCTTAGAAGCACTTAATGAGAATTTGCC | | |
| | AGAAATAAATATAGACTTAACTTACAAACAGGGACGATTACAATTCAGGCCCCCTTTT | | |
| | GAAGAAATCCGGGCTAAATATTATAGAGAAATGAAGAGATTCATCGGCATTCCAAATC | | |
| | AGTTTAAGGGAGTGGGTGAGGCCAGGAGCATTAATTCTATTTTTTCTATTATGATTGA | | |
| | TAGAAATGCAAGTGGATTTTTGACGATTTTCAGCAAAGCTGAACATCTGTTTAGAAGA | | |
| | TTGTCAGCTGTTTTACACCAACATAAGGAATGGATTGTAATTGGGCAAGTTGATATGG | | |
| | AAGCTCTGGTGGAAAAGCATCTTTTTACTGTACATGATTGGGAGAAAAATTTTAAAGC | | |
| | ATTAAAAATAAAGGGGAAAGAAGTAGAACGACTTCCAAGTGCTGTCAAGGTAGATTGT | | |
| | TTAAATATTAATTGCAACCCTGTGAAGACTGTGATTGATGATCTCATCCAGAAGTTAT | | |
| | TTGATCTGCTTGTTCTTTCTTTGAAGAAGTCCATACAGGCTCATTTACATGAAATTGA | | |
| | TACATTTGTTACTGAGGCTATGGAAGTCTTAACAATTATGCCCCAGTCTGTGGAAGAA | | |
| | ATTGGTGATGCAAATCTACAATATAGTAAGTTACAAGAACGGAAGCCAGAGATTTTGC | | |
| | CCTTATTTCAAGAAGCTGAAGACAAAAACAGACTTTTACGAACTGTGGCTGGTGGAGG | | |
| | TTTAGAAACAATTAGTAATTTGAAAGCCAAGTGGGATAAATTTGAGTTAATGATGGAA | | |
| | AGTCACCAACTTATGATTAAAGACCAGATTGAAGTGATGAAAGGAAATGTGAAATCAC | | |
| | GTCTTCAGATCTATTATCAAGAACTGGAAAAATTTAAAGCTCGTTGGGACCAACTAAA | | |
| | GCCTGGTGATGATGTTATTGAAACTGGCCAACATAATACTCTTGATAAAAGTGCAAAG | | |
| | TTAATAAAAGAGAAAAAAATTGAGTTTGATGATCTTGAAGTCACAAGAAAAAAGCTGG | | |
| | TTGATGATTGCCATCATTTTAGACTGGAAGAGCCTAATTTCTCCCTGGCAAGTAGTAT | | |
| | CTCTAAAGATATCGAGAGCTGTGCCCAAATTTGGGCCTTTTATGAAGACTTTCAACAA | | |
| | GGATTTCAGGAAATGGCCAATGAAGACTGGATCACTTTTCGGACTAAGACATACCTGT | | |
| | TTGAGGAATTTTTGATGAACTCGCATGACAGATTAAGGAAGGTTGAAGAACATTCAGT | | |
| | GATGACAGTGAAATTACAATCAGAGGTTGACAAATATAAAATCGTAATTCCTATCTTG | | |
| | AAATATGTGAGAGGGGAGCATCTTTCTCCAGATCACTGGCTTGACCTTTTTCGTCTCC | | |
| | TTGGACTTCCTAGGGGGACTAGTCTAGAGAAACTACTGTTTGGTGATTTGCTCAGAGT | | |
| | AGCTGATACAATTGTAGCCAAAGCTGCCGACCTTAAAGATTTAAATAGTCGGGCACAA | | |
| | GGTGAAGTTACAATCACAGAAGCTTTACGTGAACTTGATCTTTGGGGAGTTGGAGCAG | | |
| | TGTTTACATTAATTGATTATGAAGACAGCCAAAGTCGAACTATGAAGCTGATTAAAGA | | |
| | CTGGAAAGATATAGTAAATCAGGTTGGACATAATAGATGCCTTCTCCAATCCTTAAAG | | |
| | GATTCTCCTTATTATAAAGGATTTGAAGATAAAGTATCAATTTGGGAAAGAAAACTTG | | |
| | CAGAGTTAGATGAATACCTGCAGAATTTAAATCATATTCACAGAAAGTGGGTGTATTT | | |
| | GGAACCCATTTTCGGCCGTGGAGCATTGCCAAAAGAACAGACACGCTTCAACAGAGTT | | |
| | GATGAAGATTTTAGATCAATAATGACTGATATCAAGAAAGACAATAGAGTCACAACAT | | |
| | TAACTACTCATGCTGGAATAAGAAATTCTCTACTAACAATACTTGATCAGCTTCAAAG | | |
| | ATGTCAGAGATCATTAAATGAATTTTTGGAGGAAAAACGCTCAGCATTCCCAAGATTT | | |
| | TATTTTATTGGTGATGATGACTTATTAGAAATATTGGGCCAGTCTACCAACCCATCAG | | |
| | TGATTCAGTCTCACCTGAAGAAGCTTTTTGCTGGTATTAACAGTGTTTGCTTTGATGA | | |
| | GAAATCAAAACATATAACTGCAATGAAATCTTTAGAGGGAGAAGTTGTACCTTTTAAA | | |
| | AATAAAGTTCCTCTATCAAATAATGTAGAGACATGGTTGAATGATTTGGCCTTAGAAA | | |
| | TGAAGAAAACTTTGGAACAGTTGTTGAAGGAATGTGTTACTACTGGGCGAAGTTCTCA | | |
| | AGGTGCAGTTGACCCATCTCTGTTCCCTTCACAGATTTTATGCTTGGCGGAGCAGATT | | |
| | AAATTCACTGAAGATGTAGAAAATGCTATTAAAGATCATAGTCTTCATCAGATTGAAA | | |
| | CACAACTGGTGAATAAGTTAGAGCAATATACTAACATTGATACAAGTTCTGAGGATCC | | |
| | AGGGAATACTGAATCGGGCATCCTGGAGCTTAAACTTAAAGCCCTAATTCTTGACATT | | |
| | ATCCATAATATTGATGTGGTAAAGCAGTTAAACCAAATTCAGGTTCATACAACTGAAG | | |
| | ACTGGGCTTGGAAAAAACAACTTAGATTCTATATGAAAAGTGATCATACATGTTGTGT | | |
| | TCAAATGGTGGATTCTGAATTTCAGTATACTTATGAATATCAGGGTAATGCTTCCAAA | | |
| | CTGGTTTATACTCCACTGACAGACAAGTGCTACTTAACTCTCACTCAAGCCATGAAGA | | |
| | TGGGACTTGGAGGAAATCCTTATGGACCAGCTGGAACTGGGAAAACGGAATCAGTAAA | | |
| | GGCTTTAGGTGGACTTCTTGGAAGACAAGTTTTAGTCTTTAATTGTGATGAGGGCATC | | |
| | GATGTGAAGTCAATGGGACGAATATTTGTTGGTTTGGTGAAGTGTGGGGCCTGGGGTT | | |
| | GTTTTGATGAATTTAATAGGCTGGAAGAATCTGTACTGTCAGCAGTTTCTATGCAAAT | | |
| | CCAGACAATTCAAGATGCTTTGAAGAATCATAGAACTGTATGTGAACTGCTTGGCAAG | | |
| | GAGGTAGAAGTAAATTCTAATTCTGGAATTTTTATCACTATGAATCCTGCTGGAAAAG | | |
| | GTTATGGAGGAAGACAAAAACTCCCTGATAATCTTAAACAGCTTTTCAGGCCCGTAGC | | |
| | TATGTCTCATCCAGACAATGAGCTTATTGCAGAAGTTATTCTCTATTCGGAAGGCTTT | | |
| | AAAGACGCTAAAGTATTGAGCAGAAAATTGGTAGCTATTTTCAATCTATCTAGGGAAC | | |
| | TTTTGACACCTCAGCAACATTATGATTGGGGTTTGAGAGCTTTGAAGACAGTTCTGAG | | |
| | AGGAAGTGGAAATCTCCTTAGACAGCTAAACAAAAGTGGCACTACACAGAATGCTAAT | | |
| | GAAAGTCATATTGTGGTACAAGCACTGAGGCTTAATACAATGTCAAAGTTTACGTTTA | | |
| | CTGATTGCACCCGGTTTGATGCACTGATAAAAGATGTCTTTCCGGGAATTGAATTGAA | | |
| | AGAAGTGGAATATGATGAACTAAGTGCTGCATTAAAGCAGGTCTTTGAAGAGGCCAAT | | |
| | TATGAAATTATACCCAATCAGATCAAAAAGGCTTTAGAATTGTATGAACAGTTATGCC | | |
| | AGAGGATGGGAGTTGTTATTGTTGGTCCAAGTGGTGCTGGAAAATCAACGCTTTGGAG | | |
| | AATGTTAAGGGCTGCGCTTTGTAAAACTGGCAAAGTAGTGAAACAATATACTATGAAT | | |
| | CCCAAAGCTATCCCTCGATATCAATTATTAGGCCATATTGACATGGACACAAGAGAAT | | |
| | GGTCTGATGGTGTTTTGACAAATAGTGCTCGTCAAGTGGTTCGGGAACCTCAAGATGT | | |
| | CAGCTCATGGATAATCTGTGATGGTGATATTGACCCTGAATGGATAGAATCTCTGAAT | | |
| | TCTGTTCTGGATGATAATCGACTGCTGACTATGCCCAGTGGAGAAAGGATTCAGTTTG | | |
| | GCCCAAATGTTAACTTTGTATTTGAAACTCATGATTTAAGTTGTGCATCACCAGCCAC | | |
| | AATATCTAGAATGGGAATGATCTTTCTTAGTGATGAAGAGACAGATCTTAATTCTCTG | | |
| | ATAAAATCTTGGTTGAGGAATCAGCCTGCTGAATATAGAAATAATCTTGAAAATTGGA | | |
| | TTGGAGATTATTTTGAAAAGGCTTTACAATGGGTTCTAAAGCAGAATGACTATGTGGT | | |
| | AGAAACAAGTTTGGTTGGGACTGTGATGAATGGTTTGTCACATCTACATGGTTGCAGA | | |
| | GATCATGACGAATTCATTATTAATCTCATAAGGGGACTTGGTGGAAATCTGAATATGA | | |
| | AGTCACGTTTGGAATTTACCAAAGAGGTTTTTCATTGGGCACGAGAATCTCCTCCAGA | | |
| | CTTTCACAAACCTATGGATACCTACTATGACTCTACTAGGGGTCGATTAGCAACATAT | | |
| | GTGCTTAAGAAGCCAGAAGACTTGACTGCTGATGATTTCAGTAACGGCTTAACTCTTC | | |
| | CAGTCATTCAGACTCCTGACATGCAACGAGGTCTAGATTATTTCAAACCATGGTTAAG | | |
| | TTCTGATACTAAACAGCCCTTTATTCTCGTAGGACCAGAAGGATGTGGCAAAGGGATG | | |
| | CTGCTCAGGTACGCATTTTCACAACTCCGGTCCACTCAAATTGCTACAGTTCACTGTA | | |
| | GTGCACAAACCACTTCTCGACATCTCCTGCAGAAACTGAGCCAGACTTGCATGGTAAT | | |
| | CAGTACTAATACTGGTCGTGTATACAGACCAAAAGACTGTGAAAGACTTGTTCTGTAC | | |
| | TTAAAAGATATCAACCTACCTAAACTTGATAAATGGGGGACCAGTACTTTGGTAGCAT | | |
| | TCCTACAACAGGTATTGACGTATCAAGGATTTTATGATGAAAATTTGGAATGGGTTGG | | |
| | TCTAGAAAATATTCAAATTGTGGCTTCTATGTCAGCTGGAGGAAGACTGGGAAGACAT | | |
| | AAACTTACTACCAGATTTACTTCCATCGTTCGTCTTTGTTCTATAGATTACCCAGAAA | | |
| | GAGAGCAGTTACAAACGATTTATGGAGCATATTTGGAACCAGTTCTACATAAAAATCT | | |
| | GAAGAATCATTCTATTTGGGGTTCTTCATCAAAAATTTATCTTTTAGCAGGATCTATG | | |
| | GTACAAGTGTATCAACAGGTAGATATGCATCAGGTGCGAGCCAAATTTACAGTTGATG | | |
| | ATTATAGTCACTATTTCTTTACTCCTTGCATTCTTACCCAATGGGTTCTTGGCTTATT | | |
| | TAGATATGATTTAGAAGGAGGATCCTCAAACCATCCACTAGATTATGTGTTAGAAATT | | |
| | GTAGCATATGAGGCACGGCGCTTATTTCGTGACAAAATTGTTGGTGCAAAGGAACTTC | | |
| | ATTTATTTGACATCATTTTAACATCAGTGTTTCAAGGAGATTGGGGCTCAGACATATT | | |
| | AGACAATATGTCAGATAGTTTCTACGTTACATGGGGAGCTCGGCATAATTCAGGAGCA | | |
| | AGGGCAGCCCCAGGACAACCATTACCTCCACATGGAAAACCACTTGGAAAACTAAACT | | |
| | CTACTGATCTCAAGGATGTTATTAAAAAGGGTCTTATTCATTATGGACGAGATAACCA | | |
| | GAATTTAGACATTTTACTTTTCCACGAAGTCTTGGAGTATATGTCTAGGATAGATAGA | | |
| | GTGCTGAGTTTCCCTGCACGTTCACTTCTATTAGCAGGACGCAGTGGTGTAGGTCGTC | | |
| | GGACCATCACTTCTTTAGTCAGTCACATGCATGGAGCGGTCCTGTTTTCTCCAAAGAT | | |
| | TTCCAGAGGATATGAACTGAAGCAGTTCAAAAATGATCTCAAACATGTGCTGCAACTT | | |
| | GCAGGAATTGAAGCACAACAGGTAGTTTTACTTCTTGAGGATTACCAGTTTGTACATC | | |
| | CTACATTTTTGGAGATGATCAATAGCCTTTTGTCTTCAGGTGAAGTTCCTGGACTCTA | | |
| | TACTCTTGAAGAATTAGAGCCCTTGCTGTTACCACTTAAGGATCAAGCTTCACAAGAT | | |
| | GGTTTTTTTGGACCAGTCTTCAATTACTTCACATATAGAATTCAGCAAAACTTGCATA | | |
| | TTGTCTTGATAATGGATTCTGCAAATTCAAACTTCATGATAAACTGTGAGAGTAATCC | | |
| | AGCTTTGCATAAGAAATGCCAGCTGTTGTGGATGGAGGGTTGGTCCAATAGCAGTATG | | |
| | AAGAAAATACCTGAAATGTTATTCAGTGAAACAGGTGGTGGAGAAAAATACAATGATA | | |
| | AAAAACGAAAAGAAGAAAAGAAAAAAAATTCAGTTGATCCTGATTTTCTAAAATCATT | | |
| | TTTATTAATCCATCAATCTTGTAAAGCATATGGTGCTACACCAAGCCGATACATGACC | | |
| | TTTTTACATGTGTATTCTGCCATTAGTAGTAGCAAGAAAAAGGAATTATTAAAAAGAC | | |
| | AAAGTCATTTGCAGGCTGGTGTATCTAAACTAAATGAAGCTAAAGCTCTTGTGGATGA | | |
| | ACTGAACAGAAAAGCTGGAGAACAAAGTGTGTTACTTAAAACGAAGCAAGATGAAGCA | | |
| | GATGCTGCCCTTCAAATGATCACAGTGTCAATGCAGGATGCTAGTGAGCAAAAAACAG | | |
| | AACTTGAAAGACTGAAGCACAGAATAGCAGAAGAAGTTGTTAAAATTGAAGAAAGAAA | | |
| | AAATAAAATTGATGATGAATTAAAAGAAGTACAACCTTTAGTCAATGAAGCTAAACTA | | |
| | GCAGTTGGAAACATTAAGCCCGAATCACTTTCAGAAATTCGCTCACTACGCATGCCAC | | |
| | CTGATGTAATTAGAGATATTCTTGAAGGAGTTTTAAGGTTGATGGGTATCTTTGATAC | | |
| | ATCTTGGGTGAGCATGAAAAGTTTCCTTGCAAAAAGAGGTGTAAGACAAGACATAGCA | | |
| | ACCTTTGATGCCCGAAATATTTCAAAGGAAATAAGAGAGAGTGTTGAAGAACTTCTTT | | |
| | TTAAAAATAAAGGCTCTTTTGATCCAAAGAATGCTAAGCGTGCCAGTACTGCAGCTGC | | |
| | ACCTTTGGCTGCCTGGGTGAAAGCCAATATTCAGTATTCCCATGTCTTGGAACGAATT | | |
| | CATCCTTTGGAAACTGAACAGGCAGGATTAGAATCGAATCTGAAGAAAACTGAAGACA | | |
| | GAAAAAGGAAACTAGAGGAGCTTCTTAATTCTGTTGGTCAAAAGGTATCAGAACTCAA | | |
| | AGAAAAATTTCAGAGCAGGACTTCAGAAGCTGCCAAACTTGAGGCTGAAGTAAGCAAG | | |
| | GCACAAGAAACAATCAAAGCTGCAGAAGTCTTAATTAATCAGCTTGACAGAGAACATA | | |
| | AGAGATGGAATGCACAGGTTGTAGAGATAACAGAGGAATTAGCTACTCTTCCTAAAAG | | |
| | AGCTCAACTTGCTGCTGCATTTATTACATATCTTTCTGCTGCTCCTGAATCTCTGAGA | | |
| | AAAACCTGTTTGGAAGAATGGACCAAGTCAGCTGGTCTTGAGAAATTTGATCTGAGGA | | |
| | GATTTCTTTGTACTGAAAGTGAGCAGTTAATTTGGAAAAGTGAAGGCCTACCATCAGA | | |
| | TGACCTTTCCATAGAAAATGCTCTTGTAATATTACAGAGTCGAGTGTGCCCATTTCTT | | |
| | ATAGATCCTTCTTCCCAAGCTACAGAGTGGTTAAAAACACATTTGAAAGACTCACGTT | | |
| | TAGAAGTTATCAATCAGCAGGATAGTAACTTTATCACAGCTCTTGAATTAGCAGTACG | | |
| | TTTTGGGAAAACCCTTATTATACAAGAGATGGATGGTGTAGAACCTGTTCTTTATCCA | | |
| | TTATTGAGACGAGATCTGGTTGCTCAAGGACCACGTTATGTGGTACAAATAGGTGACA | | |
| | AAATTATTGACTACAATGAAGAATTCCGCCTCTTTTTGTCAACAAGAAACCCAAATCC | | |
| | TTTTATTCCACCGGATGCAGCTTCCATTGTTACTGAGGTTAACTTTACTACAACAAGA | | |
| | AGTGGATTACGAGGGCAGCTTTTAGCTTTAACCATTCAGCATGAGAAACCTGATTTAG | | |
| | AAGAACAGAAAACAAAACTATTACAACAGGAAGAAGATAAGAAAATACAGCTAGCCAA | | |
| | GCTCGAAGAATCTCTTCTAGAGACACTTGCCACATCTCAAGGCAATATTTTGGAAAAT | | |
| | AAGGATTTGATTGAGTCTTTGAATCAGACAAAAGCAAGCAGTGCACTTATTCAAGAGT | | |
| | CACTTAAAGAATCTTACAAACTCCAAATTTCCCTTGATCAAGAACGGGATGCCTATCT | | |
| | CCCCCTGGCTGAGAGTGCCAGCAAGATGTACTTCATTATTTCTGATTTGTCCAAAATT | | |
| | AATAACATGTACCGTTTTAGTTTGGCTGCTTTTCTCCGACTTTTCCAACGAGCTCTAC | | |
| | AAAACAAACAGGATTCTGAAAATACAGAACAGAGAATCCAGTCACTTATCAGCTCATT | | |
| | ACAACATATGGTATATGAATATATATGTCGTTGTCTATTTAAGGCTGATCAGTTGATG | | |
| | TTCGCTTTGCATTTTGTTCGAGGCATGCATCCTGAACTTTTTCAAGAAAATGAATGGG | | |
| | ATACGTTTACAGGTGTGGTTGTTGCAGACATGTTACGGAAAGCTGACTCTCAACAAAA | | |
| | AATACGTGATCAGCTTCCGTCTTGGATAGATCAGGAACGAAGCTGGGCCGTGGCAACA | | |
| | TTAAAGATTGCTCTCCCCAGTCTTTATCAGACCCTCTGCTTTGAAGATGCAGCTCTGT | | |
| | GGCGTACTTATTATAATAATTCAATGTGTGAGCAAGAGTTTCCATCTATCCTTGCAAA | | |
| | GAAAGTTTCCTTATTTCAGCAGATTCTTGTAGTACAGGCGCTAAGACCGGACAGATTG | | |
| | CAAAGTGCCATGGCTCTTTTTGCATGTAAAACTCTGGGACTGAAAGAGGTGTCCCCAC | | |
| | TGCCTCTAAATCTCAAACGTTTATACAAAGAGACACTGGAAATTGAACCCATCTTGAT | | |
| | AATTATTTCTCCGGGTGCTGATCCTTCTCAGGAACTTCAAGAACTAGCTAATGCTGAA | | |
| | AGAAGCGGAGAGTGTTATCACCAGGTTGCCATGGGTCAAGGTCAAGCTGATTTAGCAA | | |
| | TTCAAATGCTAAAAGAATGTGCCCGCAATGGAGACTGGCTCTGTTTGAAGAACTTACA | | |
| | TCTTGTGGTATCTTGGCTCCCAGTTCTGGAAAAGGAATTGAATACTCTTCAACCTAAA | | |
| | GATACCTTTCGTCTTTGGCTCACTGCAGAAGTTCATCCCAACTTTACTCCTATTTTAC | | |
| | TACAGTCAAGTCTGAAGATAACATATGAGTCACCTCCAGGTTTAGAGAACAATTTAAT | | |
| | GCGTACTTATGAGTCTTGGACTCCTGAGCAAATTAGCAAAAAAGATAATACACATCGA | | |
| | GCTCATGCTCTCTTCAGTCTTGCATGGTTTCATGCTGCATGTCAAGAAAGAAGAAACT | | |
| | ATATTCCTCAGGGTTGGACAAAGTTTTATGAATTTTCTTTATCAGATCTTCGGGCTGG | | |
| | GTACAACATTATTGACAGACTTTTTGATGGTGCCAAAGATGTACAATGGGAATTTGTA | | |
| | CATGGTTTACTTGAAAATGCTATTTATGGAGGACGTATAGACAACTATTTTGACCTTA | | |
| | GAGTTCTTCAGTCATACCTGAAGCAGTTTTTTAATTCTTCAGTTATTGATGTATTCAA | | |
| | CCAAAGGAACAAGAAAAGCATTTTTCCATATTCCGTATCTCTACCACAATCCTGCAGC | | |
| | ATTTTGGACTATCGTGCTGTCATTGAGAAAATTCCAGAGGACGACAAACCTAGTTTCT | | |
| | TTGGTCTGCCTGCCAATATCGCTCGCTCATCTCAGCGCATGATCAGTTCTCAGGTTAT | | |
| | TTCACAGTTGAGGATTTTGGGCAGATCCATAACAGCTGGTTCCAAATTTGATAGAGAA | | |
| | ATCTGGTCTAATGAACTTTCTCCTGTCCTCAATCTCTGGAAGAAACTAAACCAGAATT | | |
| | CAAACCTAATACATCAGAAAGTGCCTCCTCCTAACGATCGACAAGGATCTCCAATACT | | |
| | GTCATTCATCATTCTTGAACAATTTAATGCTATTCGTTTAGTACAAAGTGTCCACCAG | | |
| | TCTCTTGCTGCTCTCAGCAAAGTCATCAGAGGAACTACTTTACTGAGTTCAGAAGTAC | | |
| | AAAAATTGGCAAGTGCTTTATTAAACCAAAAGTGTCCTCTCGCATGGCAGAGCAAGTG | | |
| | GGAAGGCCCAGAAGATCCCTTACAATACCTGAGAGGTCTTGTTGCCCGTGCCCTTGCA | | |
| | ATACAGAACTGGGTAGATAAAGCTGAAAAACAGGCTCTTCTCTCTGAAACACTTGACC | | |
| | TATCAGAACTTTTCCATCCAGACACATTTCTTAATGCTCTTCGCCAGGAAACTGCAAG | | |
| | GGCAGTGGGTCGTTCTCTGGATAGCCTTAAATTTGTAGCCTCATGGAAAGGTCGACTG | | |
| | CAAGAAGCAAAGCTACAAATTAAGATCAGTGGCTTGTTACTAGAAGGATGTAGTTTTG | | |
| | ATGGAAATCAACTTTCTGAAAATCAGCTTGATTCTCCCAGCGTGTCATCAGTGCTCCC | | |
| | TTGTTTTATGGGCTGGATTCCACAGGATGCATGTGGTCCATATTCTCCGGATGAGTGC | | |
| | ATCTCTTTGCCTGTTTACACAAGTGCTGAAAGGGATCGTGTGGTTACCAATATTGATG | | |
| | TTCCATGTGGGGGCAACCAAGACCAGTGGATTCAGTGTGGAGCAGCTCTATTCCTAAA | | |
| | AAATCAGTAGAATCTAATGACAACAAAAGCCATCTTCACAAAAGGGAACATTGATTCT | | |
| | TTAAGCTTTAAATCAAACATGTGGTCAGTCTACATTTGAAATGTTAGTTCAAAATATT | | |
| | AACATATAGTTATGTTGTTGATGTCACTGAAATTTTAATGTGTAAAAGCAGCACTGTG | | |
| | CATCTTTTAAAGTAATAAATTAATGGAGTTATTGTTAAAACAGAGTATTCTTTTGACA | | |
| | ACATTAAATATTTCTGTGAGAAAGTTCACTTTTCCAGTGGCTCAAAAATTTGTTTTAG | | |
| | GTCAGAGATTTTAAGTGGTATATTAACCAATAATAAATATTTTGGCTGTC | | |
| | ORF Start: ATG at 61 | | ORF Stop: TAG at 13000 |
| | SEQ ID NO:312 | 4313 aa | MW at 493435.2 kD |

| | |
|---|---|
| NOV29a, | MANGTADVRKLFIFTTTQNYFGLMSELWDQPLLCNCLEINNFLDDGNQMLLRVQRSDA |
| CG154509-01 | |
| Protein Sequence | GISFSNTIEFGDTKDKVLVFFKLRPEVITDENLHDNILVSSMLESPISSLYQAVRQVF |
| | APMLLKDQEWSRNFDPKLQNLLSELEAGLGTVLRRSDTNLTKLKFKEDDTRGILTPSD |
| | EFQFWIEQAHRGNKQISKERANYFKELFETTAREFYNLDSLSLLEVVDLVETTQDVVD |
| | DVWRQTEHDHYPESRMLHLLDIIGGSFGRFVQKKLGTLNLWEDPYYLVKESLKAGISI |
| | CEQWVIVCNHLTGQVWQRYVPHPWKNEKYFPETLDKLGKRLEEVLAIRTIHEKFLYFL |
| | PASEEKIICLTRVFEPFTGLNPVQYNPYTEPLWKAAVSQYEKTIAPAEQKIAGKLKNY |
| | ISEIQDSPQQLLQAFLKYKELVKRPTISKELMLERETLLARLVDSTKDFRLDFENRCR |
| | GIPGDASGPLSGKNLSEVVNSIVWVRQLELKVDDTIKTAEALLSDLPGFRCFHQSAKD |
| | LLDQLKLYEQEQFDDWSRDIQSGLSDSRSGLCIEASSRIMELDSNDGLLKVHYSDRLV |
| | ILLREVRQLSALGFVIPAKIQQVANIAQKFCKQAIILKQVAHFYNSIDQQMIQSQRPM |
| | MLQSALAFEQIIKNSKAGSGGKSQITWDNPKELEGYIQKLQNAAERLATENRKLRKWH |
| | TTFCEKVVVLMNIDLLRQQQRWKDGLQELRTGLATVEAQGFQASDMHAWKQHWNHQLY |
| | KALEHQYQMGLEALNENLPEINIDLTYKQGRLQFRPPFEEIRAKYYREMKRFIGIPNQ |
| | FKGVGEARSINSIFSIMIDRNASGFLTIFSKAEHLFRRLSAVLHQHKEWTVIGQVDME |
| | ALVEKHLFTVHDWEKNFKALKIKGKEVERLPSAVKVDCLNINCNPVKTVTDDLIQKLF |
| | DLLVLSLKKSIQAHLHEIDTFVTEAMEVLTIMPQSVEEIGDANLQYSKLQERKPEILP |
| | LFQEAEDKNRLLRTVAGGGLETISNLKAKWDKFELMMESHQLMIKDQIEVMKGNVKSR |
| | LQTYYQELEKFKARWDQLKPGDDVIETGQHNTLDKSAKLIKEKKIEFDDLEVTRKKLV |
| | DDCHHFRLEEPNFSLASSISKDIESCAQIWAFYEEFQQGFQEMANEDWITFRTKTYLF |
| | EEFLMNWHDRLRKVEEHSVMTVKLQSEVDKYKIVIPILKYVRGEHLSPDHWLDLFRLL |
| | GLPRGTSLEKLLFGDLLRVADTIVAKAADLKDLNSRAQGEVTIREALRELDLWGVGAV |
| | FTLIDYEDSQSRTMKLIKDWKDIVNQVGDNRCLLQSLKDSPYYKGFEDKVSIWERKLA |
| | ELDEYLQNLNHIQRKWVYLEPIFGRGALPKEQTRFNRVDEDFRSIMTDIKKDMRVTTL |
| | TTHAGIRNSLLTILDQLQRCQRSLNEFLEEKRSAFPRFYFIGDDDLLEILGQSTNPSV |
| | IQSHLKKLFAGINSVCFDEKSKHITANKSLEGEVVPFKNKVPLSNNVETWLNDLALEM |
| | KKTLEQLLKECVTTGRSSQGAVDPSLFPSQILCLAEQIKETEDVENAIKDHSLHQIET |
| | QLVNKLEQYTNIDTSSEDPGNTESGILELKLKALILDIIHNIDVVKQLNQIQVHTTED |
| | WAWKKQLRFYMKSDHTCCVQMVDSEFQYTYEYQGNASKLVYTPLTDKCYLTLTQAMKM |
| | GLGGNPYGPAGTGKTESVKALGGLLGRQVLVFNCDEGIDVKSMGRIFVGLVKCGAWGC |
| | FDEFNRLEESVLSAVSMQIQTIQDALKNHRTVCELLGKEVEVNSNSGIFITMNPAGKG |
| | YGGRQKLPDNLKQLFRPVAMSHPDNELIAEVILYSEGFKDAKVLSRKLVAIFNLSREL |
| | LTPQQHYDWGLRALKTVLRGSGNLLRQLNKSGTTQNANESHIVVQALRLNTMSKFTFT |
| | DCTRFDALIKDVFPGIELKEVEYDELSAALKQVFEEANYEIIPNQIKKALELYEQLCQ |
| | RMGVVIVGPSGAGKSTLWRMLPAALCKTGKVVKQYTMNPKANPRYQLLGHIDMDTREW |
| | SDGVLTNSARQVVREPQDVSSWIICDGDIDPEWIESLNSVLDDNRLLTMPSGERIQFG |
| | PNVNFVFETHDLSCASPATISRMGMIFLSDEETDLNSLIKSWLRNQPAEYRNNLENWI |
| | GDYFEKALQWVLKQNDYVVETSLVGTVMNGLSHLHGCRDHDEFIINLIRGLCGNLNNK |
| | SRLEFTKEVFHWARESPPDFHKPMDTYYDSTRGRLATYVLKKPEDLTADDFSNGLTLP |
| | VIQTPDMQRCLDYFKPWLSSDTKQPFILVGPEGCGKCMLLRYAFSQLRSTQIATVHCS |
| | AQTTSRHLLQKLSQTCMVISTNTGRVYRPKDCERLVLYLKDINLPKLDKWGTSTLVAF |
| | LQQVLTYQGFYDENLEWVGLENIQIVASMSAGGRLGRHKLTTRFTSIVRLCSIDYPER |
| | EQLQTIYGAYLEPVLHKNLKNHSIWGSSSKIYLLAGSMVQVYEQVDMHQVRAKFTVDD |
| | YSHYFFTPCILTQWVLGLFRYDLEGGSSNHPLDYVLEIVAYEARRLFRDKIVGAKELH |
| | LFDIILTSVFQGDWGSDILDNMSDSFYVTWGARHNSGARAAPGQPLPPHGKPLGKLNS |
| | TDLKDVIKKGLIHYGRDNQNLDILLFHEVLEYMSRIDRVLSFPGGSLLLAGRSGVGRR |
| | TTTSLVSHMHGAVLFSPKISRGYELKQFKNDLKHVLQLAGIEAQQVVLLLEDYQFVHP |
| | TFLEMINSLLSSGEVPGLYTLEELEPLLLPLKDQASQDGFFGPVFNYFTYRIQQNLHI |
| | VLIMDSANSNFMINCESNPALHKKCQVLWMEGWSNSSMKKIPEMLFSETGGGEKYNDK |
| | KRKEEKKKNSVDPDFLKSFLLIHESCKAYGATPSRYMTFLHVYSAISSSKKKELLKRQ |
| | SHLQAGVSKLNEAKALVDELNRKAGEQSVLLKTKQDEADAALQMITVSMQDASEQKTE |
| | LERLKHRIAEEVVKIEERKNKIDDELKEVQPLVNEAKLAVGNIKFESLSEIRSLRMPP |
| | DVIRDILEGVLRLMGIFDTSWVSMKSFLAKRGVREDIATFDARNISKEIRESVEELLF |
| | KNKGSFDPKNAKRASTAAAPLAANVKANIQYSHVLERIHPLETEQAGLESNLKKTEDR |
| | KRKLEELLNSVGQKVSELKEKFQSRTSEAAKLEAEVSKAQETIKAAEVLINQLDREHK |
| | RWNAQVVEITEELATLPKRAQLAAAFITYLSAAPESLRKTCLEEWTKSAGLEKFDLRR |
| | FLCTESEQLIWKSEGLPSDDLSIENALVILQSRVCPFLIDPSSQATEWLKTHLKDSRL |
| | EVINQQDSNFITALELAVRFGKTLIIQEMDGVEPVLYPLLRRDLVAQGPRYVVQIGDK |
| | IIDYNEEFRLFLSTRNPNPFIPPDAASIVTEVNFTTTRSGLRGQLLALTIQHEKPDLE |
| | EQKTKLLQQEEDKKIQLAKLEESLLETLATSQGNILENKDLIESLNQTKASSALIQES |
| | LKESYKLQISLDQERDAYLPLAESASKMYFIISDLSKINNMYRFSLAAFLRLFQRALQ |
| | NKQDSENTEQRIQSLISSLQHMVYEYICRCLFKADQLMFALHFVRGMHPELFQENEWD |
| | TFTGVVVGDMLRKADSQQKIRDQLPSWIDQERSWAVATLKIALPSLYQTLCFEDAALW |
| | RTYYNNSMCEQEFPSILAKKVSLFQQILVVQALRPDRLQSAMALFACKTLGLKEVSPL |
| | PLNLKRLYKETLEIEPILIIISPGADPSQELQELANAERSGECYHQVAMGQGQADLAI |
| | QMLKECARNGDWLCLKNLHLVVSWLPVLEKELNTLQPKDTFRLWLTAEVHPNFTPILL |
| | QSSLKITYESPPGLEKNLMRTYESWTPEQISKKDNTHRAHALFSLAWFHAACQERRNY |
| | IPQCWTKFYEFSLSDLRAGYNIIDRLFDGAKDVQWEFVHGLLENAIYGGRIDNYFDLR |
| | VLQSYLKQFFNSSVIDVFNQRNKKSIFPYSVSLPQSCSILDYRAVIEKIPEDDKPSFF |
| | GLPANIARSSQRMISSQVISQLRILGRSITAGSKFDREIWSNELSPVLNLWKKLNQNS |
| | NLIHQKVPPPNDRQGSPILSFIILEQFNAIRLVQSVHQSLAALSKVIRGTTLLSSEVQ |
| | KLASALLNQKCPLAWQSKWEGPEDPLQYLRGLVARALAIQNWVDKAEKQALLSETLDL |
| | SELFHPDTFLNALRQETARAVGRSVDSLKFVASWKGRLQEAKLQIKISGLLLEGCSFD |
| | GNQLSENQLDSPSVSSVLPCFMGWIPQDACGPYSPDECISLPVYTSAERDRVVTNIDV |
| | PCGGNQDQWIQCGAALFLKNQ |

Further analysis of the NOV29a protein yielded the following properties shown in Table 29B.

TABLE 29B
Protein Sequence Properties NOV29a

| | |
|---|---|
| PSort | 0.6000 probability located in nucleus; 0.3600 probability |
| analysis: | located in mitochondrial matrix space; 0.3249 probability |
| | located in microbody (peroxisome); 0.1000 probability |
| | located in lysosome (lumen) |
| SignalP | No Known Signal Sequence Predicted |
| analysis: | |

A search of the NOV29a protein against the Geneseq database, a proprietary database that contains sequences published in patents and patent publication, yielded several homologous proteins shown in Table 29C.

TABLE 29C
Geneseq Results for NOV29a

| | | | Identities/ | |
|---|---|---|---|---|
| | | | Similarities for | |
| Geneseq | Protein/Organism/Length | NOV29a Residues/ | the Matched | Expect |
| Identifier | [Patent #, Date] | Match Residues | Region | Value |
| ABB70206 | Drosophila melanogasterpolypeptide | 55 . . . 2085 | 708/2074 (34%) | 0.0 |
| | SEQ ID NO 37410 - Drosophila | 20 . . . 2015 | 1159/2074 (55%) | |
| | melanogaster, 2055 aa. [WO200171042-A2, | | | |
| | 27 SEP. 2001] | | | |
| ABB60101 | Drosophila melanogaster polypeptide | 896 . . . 4311 | 959/3550 (27%) | 0.0 |
| | SEQ ID NO 7095 - Drosophila | 1081 . . . 4471 | 1674/3550 (47%) | |
| | melanogaster, 4472 aa. [WO200171042-A2, | | | |
| | 27 SEP. 2001] | | | |
| AAB93815 | Human protein sequence SEQ ID NO: | 3761 . . . 4313 | 551/553 (99%) | 0.0 |
| | 13606 - Homo sapiens, 553 aa. | 1 . . . 553 | 552/553 (99%) | |
| | [EP1074617-A2, 07 FEB. 2001] | | | |
| AAM79140 | Human protein SEQ ID NO 1802 - | 2193 . . . 4299 | 612/2209 (27%) | 0.0 |
| | Homo sapiens, 2166 aa. | 14 . . . 2151 | 1078/2209 (48%) | |
| | [WO200157190-A2, 09 AUG. 2001] | | | |
| AAM80124 | Human protein SEQ ID NO 3770 - | 2263 . . . 4299 | 596/2135 (27%) | 0.0 |
| | Homo sapiens, 2088 aa. | 9 . . . 2073 | 1048/2135 (48%) | |
| | [WO200157190-A2, 09 AUG. 2001] | | | |

In a BLAST search of public sequence datbases, the NOV29a protein was found to have homology to the proteins shown in the BLASTP data in Table 29D.

TABLE 29D
Public BLASTP Results for NOV29a

| | | | Identities/ | |
|---|---|---|---|---|
| Protein | | | Similarities for | |
| Accession | | NOV29a Residues/ | the Matched | Expect |
| Number | Protein/Organism/Length | Match Residues | Portion | Value |
| Q9JJ79 | Cytoplasmic dynein heavy chain - | 1 . . . 4313 | 4004/4313 (92%) | 0.0 |
| | Rattus norvegicus (Rat), 4306 aa. | 1 . . . 4306 | 4175/4313 (95%) | |
| Q27802 | Dynein heavy chain isotype 1B | 7 . . . 4313 | 2677/4338 (61%) | 0.0 |
| | (EC 3.6.1.3) - Tripneustes gratilla | 5 . . . 4318 | 3354/4338 (76%) | |
| | (Hawaian sea urchin), 4318 aa. | | | |
| Q19542 | F18C12.1 protein - Caenorhabditis | 1 . . . 4311 | 1719/4328 (39%) | 0.0 |
| | elegans, 4131 aa. | 1 . . . 4131 | 2570/4328 (58%) | |
| BAC02706 | KIAA1997 protein - Homo sapiens | 3120 . . . 4313 | 1192/1194 (99%) | 0.0 |
| | (Human), 1194 aa (fragment). | 1 . . . 1194 | 1193/1194 (99%) | |
| Q9SMH5 | Cytoplasmic dynein heavy chain | 39 . . . 3064 | 1249/3133 (39%) | 0.0 |
| | 1b - Chlamydomonas reinhardtii, | 39 . . . 3074 | 1833/3133 (57%) | |
| | 3074 aa (fragment). | | | |

PFam analysis predicts that the NOV29a protein contains the domains shown in the Table 29E.

TABLE 29E
Domain Analysis of NOV29a

| | | Identities/ | |
|---|---|---|---|
| | | Similarities for | |
| Pfam | NOV29a | the Matched | Expect |
| Domain | Match Region | Region | Value |
| PRK | 1976 . . . 2002 | 9/28 (32%) | 0.69 |
| | | 20/28 (71%) | |
| DUF164 | 3099 . . . 3307 | 52/239 (22%) | 0.15 |
| | | 112/239 (47%) | |
| Dynein_heavy | 3613 . . . 4311 | 218/790 (28%) | 9.9e−129 |
| | | 513/790 (65%) | |

### Example 30

The NOV30 clone was analyzed, and the nucleotide and encoded polypeptide sequences are shown in Table 30A.

TABLE 30A
NOV30 Sequence Analysis

| | SEQ ID NO:313 | 4292 bp | |
|---|---|---|---|
| NOV30a, | GTCAGGAGCTGCAGGATCTGGCTCGAGTCCCCTGCAGGGGCCCAGAGCAGTCCTCCCT | | |
| CG155595-01 | | | |
| DNA Sequence | CGGCATGGGGCTGGAGGCTCAGAGGCTGCCAGGGGCTGAGGAGGCCCCAGTGCGGGTT | | |
| | GCCCTGCGAGTTCGACCACTGCTGCCCAAGGAGCTGCTGCACGGGCATCAGAGCTGCC | | |
| | TGCAGGTGGAGCCAGGGCTTGGCCGCGTCACTCTGGGCCGTGACCGACACTTTGGCTT | | |
| | CCACGTGGTGCTGGCCGAGGATGCGGGGCAGGAGGCCGTGTACCAGGCCTGCGTTCAG | | |
| | CCCCTCCTTGAGGCCTTCTTCGAGGGCTTCAATGCCACTGTCTTTGCCTATGGTCAGA | | |
| | CGGGCTCAGGGAAGACATACACCATGGGGGAGGCCAGTGTGCCCTCCCTCCTTGAGGA | | |
| | TGAGCAGGGCATTGTCCCGAGGGCCATGGCCGAGGCCTTCAAGCTCATCGATGAGAAC | | |
| | GACCTGCTTGACTGTCTGGTACATGTGTCCTACCTGGAAGTGTACAAGGAGGAGTTCC | | |
| | GAGACCTGCTCGAGGTGGGCACTGCCAGCCGTGACATCCAGCTCCGGGAAGATGAGCG | | |
| | CGGGAATGTTGTGCTGTGCGGGGTGAAGGAGGTCGACGTGGAGGGCCTGGATGAGGTG | | |
| | CTGAGCCTCCTGGAGATGGGCAACGCGGCGCGGCACACGCGAGCCACGCACCTCAACC | | |
| | ACCTGTCTAGCCGCTCACACACGGTCTTCACCGTGACCCTGGAGCAGCGGGGGCGCGC | | |
| | CCCCAGCCGCCTACCCCGCCCCGCCCCGGGCCAGCTGCTCGTCTCCAAGTTCCACTTC | | |
| | GTGGACCTGGCGGGCTCAGAGAGGGTGCTCAAGACGGGCAGCACCGGCGAGCGGCTCA | | |
| | AGGAGAGCATCCAGATCAACAGCAGCCTCCTGGCGCTGGGCAACGTCATCAGCGCCCT | | |
| | GGGGGACCCTCAGCGCCGGGGCAGCCACATACCCTACCGCGACTCCAAGATCACCCGG | | |
| | ATCCTCAAAGACTCGCTGGGCGGGAACGCCAAGACGGTGATGATCGCCTGCGTCAGCC | | |
| | CTTCCTCCTCCGACTTCGACGAGACCCTCAACACCCTCAACTACGCCAGCCGCGCCCA | | |
| | GAACATCCGCAACCGCGCCACGGTCAACTGGCGGCCCGAGGCCGAGCGGCCACCCGAA | | |
| | GAGACGGCGAGCGGCGCGCGGGGTCCGCCACGGCACCGCTCCGAGACCCGCATCATCC | | |
| | ACCGCGGCCGGCGCGCCCCAGGCCCAGCCACCGCCTCCGCGGCGGCCGCCATGCGCCT | | |
| | GGGCGCCGAGTGCGCGCGCTACCGGGCCTGCACCGACGCCGCCTACAGCCTCTTGCGC | | |
| | GAGCTGCAGGCCGAGCCCGGGCTGCCCGGCGCCGCCGCCCGCAAGGTGCGCGACTGGC | | |
| | TGTGCGCCGTCGAGGGCGACCGCAGCGCCCTGAGCTCCGCCTCCGGGCCCGATAGCGG | | |
| | CATCGAGAGCGCCTCCGTCGAGGACCAGGCGGCGCAGGGGGCCCGCGGCCGAAAGGTG | | |
| | GCCGAGGGACAGGAGGATGAGGGGGCGCAGCAGCTGCTGACCCTGCAGAACCAGGTGG | | |
| | CGCGGCTGGAGGAGGAGAACCGAGACTTTCTGGCTGCGCTGGAGGACGCCATGGAGCA | | |
| | GTACAAACTGCAGAGCGACCGGCTGCGTGAGCAGCAGGAGGAGATGGTGGAACTGCGG | | |
| | CTGCGGTTAGAGCTGGTGCGGCCAGGCTGGGGCGGCCCGCGGCTCCTGAATGGCCTGC | | |
| | CTCCCGGGTCCTTTGTGCCTCGACCTCATACAGCCCCCCTGGGGGGTGCCCACGCCCA | | |
| | TGTGCTGGGCATGGTGCCGCCTGCCTGCCTCCCTGGAGATGAAGTTGGCTCTGAGCAG | | |
| | AGGGGACAGGTGACAAATGGCAGGGAGGCTGGAGCTGAGTTGCTGACTGAGGTGAACA | | |
| | GGCTGGGAAGTGGCTCTTCAGCTGCTTCAGAGGAGGAAGAGGAGGAGGAGGAGCCGCC | | |
| | CAGGCGGACCTTACACCTGCGCAGTTGGGGCAGCAACCTTGACAGGCTGCCTGTTGCA | | |
| | GCAGTTGGTGGGAGCAAGGCCCGAGTTCAGGCCCGCCAGGTCCCCCCTGCCACAGCCT | | |
| | CAGAGTGGCGGCTGGCCCAGGCCCAGCAGAAGATCCGGGAGCTGGCTATCAACATCCG | | |
| | CATGAAGGAGGAGCTTATTGGCGAGCTGGTCCGCACAGGAAAGGCAGCTCAGGCCCTG | | |
| | AACCGCCAGCACAGCCAGCGTATCCGGGACCTGCAGCAGGAGGCAGAGCAGGTGCGGG | | |
| | CCGAGCTGAGTGAAGGCCAGAGGCAGCTGCGGGAGCTCGAGGGCAAGGAGCTCCAGGA | | |
| | TGCTGGCGAGCGGTCTCGGCTCCAGGAGTTCCGCACGAGGGTCGCTGCGGCCCAGAGC | | |
| | CAGGTGCAGGTGCTGAAGGAGAAGAAGCAGGCTACGGAGCGGCTGGTGTCACTGTCGG | | |
| | CCCAGAGTGAGAAGCGACTGCAGGAGCTCGAGCGGAACGTGCAGCTCATGCGGCAGCA | | |
| | GCAGGGACAGCTGCAGAGGCGGCTTCGCGAGGAGACGGAGCAGAAGCGGCGCCTGGAG | | |
| | GCAGAAATGAGCAAGCGGCAGCACCGCGTCAAGGAGCTGGAGCTGAAGCATGAGCAAC | | |
| | AGCAGAAGATCCTGAAGATTAAGACGGAAGAGATCGCGGCATTCCAGAGGAAGAGGCG | | |
| | CAGTGGCAGCAACGGCTCTGTGGTCAGCCTGGAACAGCAGCAGGTGGGGCCAGGCTGT | | |
| | GTCCGCACCCAGGGCTCCCCTGGGGGCTGGCTGGTGGGTGCACCTTTCTCCCCAGTGA | | |
| | ACCTCGAGTGGCCGCTGACACACCCAGAGAAGATTGAGGAGCAGAAGAAGTGGCTGGA | | |
| | CCAGGAGATGGAGAAGGTGCTACAGCAGCGGCGGGCGCTGGAGGAGCTGGGGGAGGAG | | |
| | CTCCACAAGCGGGAGGCCATCCTGGCCAAGAAGGAGGCCCTGATGCAGGAGAAGACGG | | |
| | GGCTGGAGAGCAAGCGCCTGAGATCCAGCCAGGCCCTCAACGAGGACATCGTGCGAGT | | |
| | GTCCAGCCGGCTCGAGCACCTGGAGAAGGAGCTGTCCGAGAAGAGCGGGCAGCTGCGG | | |
| | CAGGGCAGCGCCCAGAGCCAGCAGCAGATCCGCGGGGAGATCGACAGCCTGCGCCAGG | | |
| | AGAAGGACTCGCTGCTCAAGCAGCGCCTGGAGATCGACGGCAAGCTGAGGCAGGGGAG | | |
| | TCTGCTGTCCCCCGAGGAGGAGCGGACGCTGTTCCAGTTGGATGAGGCCATCGAGGCC | | |
| | CTGGATGCTGCCATTGAGTATAAGAATGAGGCCATCACATGCCGCCAGCGCGTGCTTC | | |
| | GGGCCTCAGCCTCGTTGCTGTCCCAGTGCGAGATGAACCTCATGCCCAAGCTCAGCTA | | |
| | CCTCTCATCCTCAGAGACCAGAGCCCTCCTCTGCAAGTATTTTGACAAGGTGGTGACG | | |
| | CTCCGAGAGGAGCAGCACCAGCAGCAGATTGCCTTCTCGGAACTGGAGATGCAGCTGG | | |
| | AGGAGCAGCAGAGGCTGGTGTACTGGCTGGAGGTGGCCCTGGAGCGGCAGCGCCTGGA | | |
| | GATGGACCGCCAGCTGACCCTGCAGCAGAAGGAGCACGAGCAGAACATGCAGCTGCTC | | |
| | CTGCAGCAGAGTCGAGACCACCTCGGTGAAGGGTTAGCAGACAGCAGGAGGCAGTATG | | |
| | AGGCCCGGATTCAAGCTCTGGAGAAGGAACTGGGCCGTTACATGTGGATAAACCAGGA | | |
| | ACTGAAACAGAAGCTCGGCGGTGTGAACGCTGTAGGCCACAGCAGGGGTGGGGAGAAG | | |
| | AGGAGCCTGTGCTCGGAGGGCAGACAGGCTCCTGGAAATGAAGATGAGCTCCACCTGG | | |
| | CACCCGAGCTTCTCTGGCTGTCCCCCCTCACTGAGGGGGCCCCCCGCACCCGGGAGGA | | |
| | GACGCGGGACTTGGTCCACGCTCCGTTACCCTTGACCTGGAAACGCTCGAGCCTGTGT | | |
| | GGGGACTCTTCAACAACACCAATATCAGGACCAGGATCAGAGGACCTCGAGGAACCAC | | |
| | ATGCACAAGGATTATTCCATACCACTTGTAATTAACACTTATTAAGGAGACAGGCAGC | | |
| | TTCTCACTTAACAAGATCACAAAGATCACAGGGTCTGATAACACCAGTGCTGCTATTC | | |
| | TGAAATGTGGTACCTTTGTTCTTCTTGAAGTTGTCAAGTTTATCCTCTAGACCATCCA | | |
| | CAGCTGACACAGAATGGCTTCTAGGCAACCCCCGCTTTAGTGATCTCTTTGAAGGGGA | | |
| | AAGCAATTCCTGGTTGAAAAGATTTCTTCGAACTTTGGTCACTTCTAAAAGCATCAAA | | |
| | ORF Start: ATG at 63 | | ORF Stop: TAA at 4035 |
| | SEQ ID NO:314 | 1324 aa | MW at 148066.3 kD |

| | |
|---|---|
| NOV30a, | MGLEAQRLPGAEEAPVRVALRVRPLLPKELLHGHQSCLQVEPGLGRVTLGRDRHFGFH |
| CG155595-01 | |
| Protein Sequence | VVLAEDAGQEAVYQACVQPLLEAFFEGFNATVFAYGQTGSGKTYTMGEASVASLLEDE |
| | QGIVPRAMAEAFKLIDENDLLDCLVHVSYLEVYKEEFRDLLEVGTASRDIQLREDERG |
| | NVVLCGVKEVDVEGLDEVLSLLEMGNAARHTGATHLNHLSSRSHTVFTVTLEQRGRAP |
| | SRLPRPAPGQLLVSKFHFVDLAGSERVLKTGSTGERLKESIQINSSLLALGNVISALG |
| | DPQRRGSHIPYRDSKITRILKDSLGGNAKTVMIACVSPSSSDFDETLNTLNYASRAQN |
| | IRNRATVNWRPEAERPPEETASGARGPPRHRSETRIIHRGRRAPGPATASAAAANRLG |
| | AECARYRACTDAAYSLLRELQAEPGLPGAAARKVRDWLCAVEGERSALSSASGPDSGI |
| | ESASVEDQAAQGAGGRKVAEGQEDEGAQQLLTLQNQVARLEEENRDFLAALEDAMEQY |
| | KLQSDRLREQQEEMVELRLRLELVRPGWGGPRLLNGLPPGSFVPRPHTAPLGGAHAHV |
| | LGMVPPACLPGDEVGSEQRGEVTNGREAGAELLTEVNRLGSGSSAASEEEEEEEEPPR |
| | RTLHLRSWGSNLDRLPVAAVGGSKARVQARQVPPATASEWRLAQAQQKIRELAINIRM |
| | KEELIGELVRTGKAAQALNRQHSQRIRELEQEAEQVRAELSEGQRQLRELEGKELQDA |
| | GERSRLQEFRRRVAAAQSQVQVLKEKKQATERLVSLSAQSEKRLQELERNVQLMRQQQ |
| | GQLQRRLREETEQKRRLEAEMSKRQHRVKELELKHEQQQKILKIKTEEIAAFQRKRRS |
| | GSNGSVVSLEQQQVGPGCVRTQGSPGGWLVGAPFSPVNLEWRLTQPEKIEEQKKWLDQ |
| | EMEKVLQQRRALEELGEELHKREAILAKKEALMQEKTGLESKRLRSSQALNEDIVRVS |
| | SRLEHLEKELSEKSGQLRQGSAQSQQQIRGEIDSLRQEKDSLLKQRLEIDGKLRQGSL |
| | LSPEEERTLFQLDEAIEALDAAIEYKNEAITCRQRVLRASASLLSQCEMNLMAKLSYL |
| | SSSETRALLCKYFDKVVTLREEQHQQQIAFSELEMQLEEQQRLVYWLEVALERQRLEM |
| | DRQLTLQQKEHEQNMQLLLQQSRDHLGEGLADSRRQYEARIQALEKELGRYMWINQEL |
| | KQKLGGVNAVGNSRGGEKRSLCSEGRQAPGNEDELHLAPELLWLSPLTEGAPRTREET |
| | RDLVHAPLPLTWKRSSLCGDSSTTPISGPGSEDLEEPHAQGLFHTTCN |

Further analysis of the NOV30a protein yielded the following properties shown in Table 30B.

TABLE 30B
Protein Sequence Properties NOV30a

| | |
|---|---|
| PSort | 0.8800 probability located in nucleus; 0.3000 probability |
| analysis: | located in microbody (peroxisome); 0.1000 probability |
| | located in mitochondrial matrix space; 0.1000 probability |
| | located in lysosome (lumen) |
| SignalP | No Known Signal Sequence Predicted |
| analysis: | |

A search of the NOV30a protein against the Geneseq database, a proprietary database that contains sequences published in patents and patent publication, yielded several homologous proteins shown in Table 30C.

TABLE 30C
Geneseq Results for NOV30a

| | | | Identities/ | |
|---|---|---|---|---|
| | | | Similarities for | |
| Geneseq | Protein/Organism/Length | NOV30a Residues/ | the Matched | Expect |
| Identifier | [Patent #, Date] | Match Residues | Region | Value |
| AAU86160 | Human PRO539 polypeptide - Homo sapiens, | 519 . . . 1301 | 734/811 (90%) | 0.0 |
| | 830 aa. [WO200153486-A1, 26 JUL. 2001] | 1 . . . 777 | 737/811 (90%) | |
| AAY96730 | PRO539, a Costal-2 homologue - Homo | 519 . . . 1301 | 734/811 (90%) | 0.0 |
| | sapiens, 830 aa. [WO200036102-A2, | 1 . . . 777 | 737/811 (90%) | |
| | 22 JUN. 2000] | | | |
| ABB81633 | Human kinesin motor protein HsKif7 | 11 . . . 354 | 341/344 (99%) | 0.0 |
| | fragment SEQ ID NO: 2 - Homo sapiens, | 1 . . . 342 | 342/344 (99%) | |
| | 342 aa. [US6395527-B1, 28 MAY 2002] | | | |
| ABB81634 | Human kinesin motor protein HsKif7 | 12 . . . 350 | 336/339 (99%) | 0.0 |
| | fragment SEQ ID NO: 4 - Homo sapiens, | 1 . . . 337 | 337/339 (99%) | |
| | 337 aa. [US6395527-B1, 28 MAY 2002] | | | |
| ABB80078 | Human kinesin motor protein (HsKrp5) | 676 . . . 1222 | 259/548 (47%) | e−131 |
| | amino acid sequence - Homo sapiens, | 593 . . . 1102 | 386/548 (70%) | |
| | 1279 aa. [US6379941-B1, 30 APR. 2002] | | | |

In a BLAST search of public sequence datbases, the NOV30a protein was found to have homology to the proteins shown in the BLASTP data in Table 30D.

TABLE 30D
Public BLASTP Results for NOV30a

| | | | Identities/ | |
|---|---|---|---|---|
| Protein | | | Similarities for | |
| Accession | | NOV30a Residues/ | the Matched | Expect |
| Number | Protein/Organism/Length | Match Residues | Portion | Value |
| Q95LL1 | Hypothetical 98.5 kDa protein - | 12 . . . 825 | 359/877 (40%) | e−166 |
| | Macaca fascicularis (Crab eating | 2 . . . 865 | 527/877 (59%) | |
| | macaque) (Cynomolgus monkey), | | | |
| | 865 aa (fragment). | | | |
| Q9UF54 | Hypothetical 96.7 kDa protein - | 676 . . . 1222 | 256/548 (46%) | e−129 |
| | Homo sapiens (Human), 833 aa | 147 . . . 656 | 384/548 (69%) | |
| | (fragment). | | | |
| Q9QXL2 | Kif21a - Mus musculus (Mouse), | 8 . . . 356 | 178/377 (47%) | 2e−88 |
| | 1573 aa. | 2 . . . 378 | 236/377 (62%) | |
| Q9CTY0 | Kinesin family member 21A - | 5 . . . 356 | 178/380 (46%) | 1e−87 |
| | Mus musculus (Mouse), 647 aa | 82 . . . 461 | 236/380 (61%) | |
| | (fragment). | | | |
| Q9NXU4 | CDNA FLJ20052 fis, clone | 8 . . . 356 | 175/377 (46%) | 8e−87 |
| | COL00777 - Homo sapiens | 2 . . . 378 | 237/377 (62%) | |
| | (Human), 576 aa (fragment). | | | |

PFam analysis predicts that the NOV30a protein contains the domains shown in the Table 30E.

TABLE 30E
Domain Analysis of NOV30a

| | | Identities/ | |
|---|---|---|---|
| | | Similarities for | |
| Pfam | NOV30a | the Matched | Expect |
| Domain | Match Region | Region | Value |
| kinesin | 21 . . . 364 | 168/404 (42%) | 1.3e−125 |
| | | 260/404 (64%) | |
| DUF164 | 681 . . . 913 | 55/251 (22%) | 0.015 |
| | | 132/251 (53%) | |

### Example 31

The NOV31 clone was analyzed, and the nucleotide and encoded polypeptide sequences are shown in Table 31A.

TABLE 31A
NOV31 Sequence Analysis

| | SEQ ID NO:315 | 5460 bp | |
|---|---|---|---|
| NOV31a, | ATGTCGGGAGCCTCAGTGAAGGTGGCTGTCCGGGTAAGGCCCTTCAATTCTCGAGAGA | | |
| CG155962-01 | | | |
| DNA Sequence | CCAGCAAGGAATCCAAATGCATCATTCAGATGCAAGGCAACTCGACCAGTATTATTAA | | |
| | CCCAAAGAATCCAAAGGAAGCTCCAAAGTCCTTCAGCTTCGACTATTCCTACTGGTCT | | |
| | CATACCTCACCCGAAGATCCCTGTTTTGCATCTCAAAACCGTGTGTACAATGACATTG | | |
| | GCAAGGAAATGCTCTTACACGCCTTTGAGGGATATAATGTCTGTATTTTTGCCTATGG | | |
| | GCAGACTGGTGCTGGAAAATCTTATACAATGATGGGTAAACAAGAACAAAGCCAGGCT | | |
| | GGCATCATTCCACAGTTATGTGAAGAACTTTTTGAGAAAATCAATGACAACTGTAATG | | |
| | AAGAAATGTCTTACTCTGTAGAGGTGAGTTACATGGAAATTTACTGTGAAAGAGTACG | | |
| | AGATTTGCTGAATCCAAAAAACAAGGGTAATTTGCGTGTGCGTGAACACCCACTTCTT | | |
| | GGACCCTATGTGGAGGATCTGTCCAAGTTGGCAGTTACTTCCTACACAGACATTGCTG | | |
| | ACCTCATGGATGCTGGGAACAAAGCCACGACAGTGGCAGCTACAAACATGAATGAAAC | | |
| | AAGTAGCCGTTCCCACGCTGTGTTTACGATTGTTTTCACCCAGAAGAAACACGATAAT | | |
| | GAGACCAACCTTTCCACTGAGAAGGTAGTCAGTAAAATCAGCTTGGTGGATCTAGCAG | | |
| | GAAGTGAACGAGCTGATTCAACTGGTGCCAAAGGGACTCGATTAAAGGAAGGAGCAAA | | |
| | TATTAATAAGTCTCTTACAACTTTGGGCAAAGTCATTTCAGCCTTGCCCGAGGTGAGT | | |
| | AAAAAGAAGAAGAAAACAGATTTTATTCCCTACAGGGATTCTGTACTTACTTGGCTCC | | |
| | TTCGAGAAAATTTAGGTGGCAATTCTCGGACTGCAATCGTTGCTGCTCTGAGCCCCGC | | |
| | GGATATCAACTACGATGAGACTTTGAGCACTCTGAGGTACGCAGATCGTGCAAAACAA | | |
| | ATTAAATGCAATGCTGTTATCAATGAGGACCCCAATGCCAAACTGGTTCGTGAATTAA | | |
| | AGGAGGAGGTGACACGGCTGAAGGACCTTCTTCGTGCTCAGGGCCTGGCAGATATTAT | | |
| | TGATGTTGATCCATTGATCGATGATTACTCTGGAAGTGGAAGCAAACTGAAAGATTTT | | |
| | CAGAACAATAAGCATAGATACTTGCTAGCCTCTGAGAATCAACGCCCTGGCCATTTTT | | |
| | CCACAGCATCCATGGGGTCCCTCACTTCATCCCCATCTTCCTGCTCACTCAGTAGTCA | | |
| | GGTGGGCTTGACGTCTGTGACCAGTATTCAACAGAGGATCATGTCTACACCTGGAGGA | | |
| | GAGGAAGCTATTGAACGTTTAAAGGAATCAGAGAAGATCATTGCTGAGTTGAATGAAA | | |
| | CTTGGGAAGAGAAGCTTCGTAAAACAGAGGCCATCAGAATGGAGAGGGAGGCTTTGTT | | |
| | GGCTGAGATGGGAGTTGCCATTCGGGAAGATGGAGGAACCCTAGGGGTTTTCTCACCT | | |
| | AAAAAGACCCCACATCTTGTTAACCTCAATGAAGACCCACTAATGTCTGAGTGCCTAC | | |
| | TTTATTACATCAAAGATGGAATTACAAGGGTTGGCCAAGCAGATGCTGAGCGGCGCCA | | |
| | GGACATAGTGCTGAGCGGGGCTCACATTAAAGAAGAGCATTGTATCTTCCGGAGTGAG | | |
| | AGAAGCAACAGCGGGGAAGTTATCGTGACCTTAGAGCCCTGTGAGCGCTCAGAAACCT | | |
| | ACGTAAATGGCAAGAGGGTGTCCCAGCCTGTTCAGCTGCGCTCAGGTAACCGTATCAT | | |
| | CATGGGTAAAAACCATGTTTTCCGCTTTAACCACCCGGAACAAGCACGAGCTGAGCGA | | |
| | GAGAAGACTCCTTCTGCTGAGACCCCCTCTGAGCCTGTGGACTGGACATTTGCCCAGA | | |
| | GGGAGCTTCTGGAAAAACAAGGAATTGATATGAAACAAGAGATGGAGAAAAGGCTACA | | |
| | GGAAATGGAGATCTTATACAAAAAGGACAAGGAAGAAGCAGATCTTCTTTTGGAGCAG | | |
| | CAGAGACTGGACTATGAGAGTAAATTGCAGGCCTTGCAGAAGCAGGTTGAAACCCGAT | | |
| | CTCTGGCTGCAGAAACAACTGAAGAGGAGGAAGAAGAGGAAGAAGTTCCTTGGACACA | | |
| | GCATGAATTTGAGTTGGCCCAATGGGCCTTCCGGAAATGGAAGTCTCATCAGTTTACT | | |
| | TCATTACGGGACTTACTCTGGGGCAATGCCGTGTACCTAAAGGAGGCCAATGCCATCA | | |
| | GTGTGGAACTGAAAAAGAAGGTACAGTTTCAGTTTGTTCTGCTGACTGACACACTGTA | | |
| | CTCCCCTTTGCCTCCTGAATTACTTCCCACTGAGATGGAAAAAACTCATGAGGACAGG | | |
| | CCTTTCCCTCGCACAGTGGTAGCAGTAGAAGTCCAGGATTTGAAGAATGGAGCAACAC | | |
| | ACTATTGGTCTTTGGAGAAACTCAAGCAGAGGCTGGATTTGATGCGAGAGATGTATGA | | |
| | TAGGGCAGGGGAGATGGCCTCCAGTGCCCAAGACGAAAGCGAAACCACTGTGACTGGC | | |
| | AGCGATCCCTTCTATGATCGGTTCCACTGGTTCAAACTTGTGGGGAGCTCCCCCATTT | | |
| | TCCACGGCTGTGTGAACGAGCGCCTTGCCGACCGCACACCCTCCCCCACTTTTTCCAC | | |
| | GGCCGATTCCGACATCACTGAGCTGGCTGACGAGCAGCAAGATGAGATGGAGGATTTT | | |
| | GATGATGAGGCATTCGTGGATGACGCCGGCTCTGACGCAGGGACGGAGGAGGGATCAG | | |
| | ATCTCTTCAGTGACGGGCATGACCCGTTTTACGACCGATCCCCTTGGTTCATTTTAGT | | |
| | GGGAAGGGCATTTGTTTACCTGAGCAATCTGCTGTATCCCGTGCCCCTGATCCACAGG | | |
| | GTGGCCATCGTCAGTGAGAAAGGTGAAGTGCGGGGATTTCTGCGTGTGGCTGTACAGG | | |
| | CCATCGCAGATGAAGAAGCTCCTGATTATGGCTCTGGAATTCGACAGTCAGGAACAGC | | |
| | TAAAATATCTTTTGATAATGAATACTTTAATCAGAGTGACTTTTCGTCTGTTGCAATG | | |
| | ACTCGTTCTGGTCTGTCCTTGGAGGAGTTGAGGATTGTGGAAGGACAGGGTCAGAGTT | | |
| | CTGAGGTCATCACTCCTCCAGAAGAAATCAGTCGAATTAATGACTTGTTAGATTTGAA | | |
| | GTCAAGCACTTTGCTGGATGGTAAGATGGTAATGGAAGGGTTTTCTGAAGAGATTGGC | | |
| | AACCACCTGAAACTGGGCAGTGCCTTCACTTTCCGAGTAACAGTGTTGCAGGCCAGTG | | |
| | GAATCCTCCCAGAGTATGCAGATATCTTCTGTCAGTTCAGCTTTTTGCATCGCCATGA | | |
| | TGAAGCATTCTCCACGGAGCCCCTCAAAAACAATGGCAGAGGAAGTCCCCTGGCCTTT | | |
| | TATCATGTGCAGAATATTGCAGTGGAGATCACTGAATCATTTGTGGATTACATCAAAA | | |
| | CCAAGCCTATTGTATTTGAAGTCTTTGGGCATTATCAGCAGCACCCACTTCATCTGCA | | |
| | AGGACAGGAGCTTAACAGTCCGCCTCAGCCGTGCCGCCGATTCTTCCCTCCACCCATG | | |
| | CCACTGTCCAAGCCAGTTCCAGCCACCAAGTTAAACACGATGAGCAAAACCAGCCTTG | | |
| | GCCAGAGCATGAGCAAGTATGACCTCCTGGTTTGGTTTGAGATCAGTGAACTGGAGCC | | |
| | TACAGGAGAGTATATCCCAGCTGTGGTTGACCACACAGCAGGCTTGCCTTGCCAGGGG | | |
| | ACATTTTTGCTTCATCAGGGCATCCAGCGAAGGATCACAGTGACCATTATCCATGAGA | | |
| | AGGGGAGCGAGCTCCATTGGAAAGATGTTCGTGAACTGGTGGTAGGTGGTCGTATTCG | | |
| | GAATAACCCTGAGGTGGATGAAGCTGCAGTTGATGCCATCCTCTCCCTAAATATTATT | | |
| | TCTGCCAAGTACCTGAAGTCTTCCCACAACTCTAGCAGGACCTTCTACCGCTTTGAGG | | |
| | CTGTGTGGGATAGCTCTCTGCATAACTCCCTTCTTCTGAACCGAGTGACACCCTATGG | | |
| | AGAAAAGATCTACATGACCTTGTCGGCCTACCTAGAGCTGGATCATTGCATCCAGCCG | | |
| | GCTGTCATCACCAAGGATGTGTGCATGGTCTTCTACTCCCGAGATGCCAAGATCTCAC | | |
| | CACCACGCTCTCTGCGTAGCCTCTTTGGCAGCGGCTACTCAAAGTCACCAGATTCGAA | | |
| | TCGAGTCACTGGCATTTACGAACTCAGCTTATGCAAAATGTCAGACACAGGTAGTCCA | | |
| | GGTAAGATGCAGAGAAGGAGAAGAAAAATCTTAGATACGTCAGTGGCATATGTGCGGG | | |
| | GAGAAGAGAACTTAGCAGGCTGGCGGCCCCGTGGAGACAGCCTCATCCTTGAGCACCA | | |
| | GTGGGAGCTGGAGAAGCTGGAAAAAACCCGCCACTTTTTGCTGCTGCGTGAGAGACTT | | |
| | GGTGACAGCATCCCCAAATCCCTGAGCGACTCGTTATCCCCCAGCCTCAGCAGTGGGA | | |
| | CCCTCAGCACCTCCACCACTATCTCCTCTCAGATCTCAACCACTACCTTTGAAAGCGC | | |
| | CATCACACCTAGCGAGAGCAGTGGCTATGATTCAGGAGACATCGAAAGCCTGGTGGAC | | |
| | CGAGAGAAAGAGCTGGCTACCAAGTGCCTGCAACTTCTCACCCACACTTTCAACAGAG | | |
| | AATTCAGCCAGGTGCACGGCAGCGTCACTGACTGTAAGGTGAGCGATATCTCTCCAAT | | |
| | TGGACGGGATCCCTCTGAGTCCAGTTTCAGCAGTGCCACCCTCACTCCCTCCTCCACC | | |
| | TGTCCCTCTCTGGTAGACTCTAGGAGCAACTCTCTGGATCAGAAGACCCCAGAAGCCA | | |
| | ATTCCCGGGCCTCTAGTCCCTGCCCAGAATTTGAACAGTTTCAGATTGTCCCAGCTGT | | |
| | GGAAACACCATATTTGGCCCGAGCAGGAAAAAACGAATTTCTCAATCTTGTTCCAGAT | | |
| | ATTGAAGAAATTAGATCAGTGGTCTCTAAGAAAGGATACCTTCATTTCAAGGAGCCTC | | |
| | TTTACAGTAACTGGGCTAAACATTTTGTTGTCGTCCGTCGGCCTTATGTCTTCATCTA | | |
| | TAACAGTGACAAAGACCCTGTGGAGCGTGGAATCATTAACCTGTCCACAGCACAGGTG | | |
| | GAGTACAGTGAGGACCAGCAGGCCATGGTGAAGACACCAAACACCTTTGCTGTCTGCA | | |
| | CAAAGCACCGTGGGGTCCTTTTGCAGGCCCTCAATGACAAAGACATGAACGACTGGTT | | |
| | GTATCCCTTCAACCCACTTCTAGCTGGCACAATACGGAGGTCAAAGCTTTCCCGCAGA | | |
| | TGCCCGAGCCAGTCGAAATACTAAGTGACTCTGCCGAGTGCCCTCACTCGCCTTCGAG | | |
| | AGATAAAG | | |
| | ORF Start: ATG at 1 | | ORF Stop: TAA at 5416 |
| | SEQ ID NO:316 | 1805 aa | MW at 203184.5 kD |

| | |
|---|---|
| NOV31a, | MSGASVKVAVRVRPFMSRETSKESKCIIQMQGNSTSIINPKWPKEAPKSFSFDYSYWS |
| CG155962-01 | |
| Protein Sequence | HTSPEDPCFASQNRVYNDIGKEMLLHAFEGYNVCIFAYGQTGAGKSYTMMGKQEESQA |
| | GIIPQLCEELFEKINDNCNEEMSYSVEVSYMEIYCERVRDLLNPKNKGNLRVREHPLL |
| | GPYVEDLSKLAVTSYTDIADLMDAGNKARTVAATNMNETSSRSHAVFTIVFTQKKHDN |
| | ETNLSTEKVVSKISLVDLAGSERADSTGAKGTRLKEGANINKSLTTLGKVISALAEVS |
| | KKKKKTDFIPYRDSVLTWLLRENLGGNSRTANVAALSPADINYDETLSTLRYADRAKQ |
| | IKCNAVINEDPNAKLVRELKEEVTRLKDLLRAQGLGDIIDVDPLIDDYSGSGSKLKDF |
| | QNNKHRYLLASENQRPGHFSTASMGSLTSSPSSCSLSSQVGLTSVTSIQERIMSTPGG |
| | EEAIERLKESEKIIAELNETWEEKLRKTEAIRMEREALLAEMGVAIREDGGTLGVFSP |
| | KKTPHLVNLNEDPLMSECLLYYIKDGITRVGQADAERRQDIVLSGAHIKEEHCIFRSE |
| | RSNSGEVIVTLEPCERSETYVNGKRVSQPVQLRSGNRIIMGKNHVFRFNHPEQARAER |
| | EKTPSAETPSEPVDWTFAQRELLEKQGIDMKQEMEKRLQEMEILYKKEKEEADLLLEQ |
| | QRLDYESKLQALQKQVETRSLAAETTEEEEEEEEVPWTQHEFELAQWAFRKWKSHQFT |
| | SLRDLLWGNAVYLKEANAISVELKKKVQFQFVLLTDTLYSPLPPELLPTEMEKTHEDR |
| | PFPRTVVAVEVQDLKNGATHYWSLEKLKQRLDLMREMYDRAGEMASSAQDESETTVTG |
| | SDPFYDRFHWFKLVGSSPIFHGCVNERLADRTPSPTFSTADSDITELADEQQDEMEDF |
| | DDEAFVDDAGSDAGTEEGSDLFSDGHDPFYDRSPWFILVGRAFVYLSNLLYPVPLIHR |
| | VAIVSEKGEVRGFLRVAVQAIADEEAPDYGSGIRQSGTAKISFDNEYFNQSDFSSVAM |
| | TRSGLSLEELRIVEGQGQSSEVITPPEEISRIMDLLDLKSSTLLDGKMVMEGFSEEIG |
| | NHLKLGSAFTFRVTVLQASGILPEYADIFCQFSFLHRHDEAFSTEPLKNNGRGSPLAF |
| | YHVQNIAVEITESFVDYIKTKPIVFEVFGHYQQHPLHLQGQELNSPPQPCRRFFPPPM |
| | PLSKPVPATKLNTMSKTSLGQSMSKYDLLVWFEISELEPTGEYIPAVVDHTAGLPCQG |
| | TFLLHQGIQRRITVTIIHEKGSELHWKDVRELVVGGRIRNKPEVDEAAVDAILSLNII |
| | SAKYLKSSHNSSRTFYRFEAVWDSSLHNSLLLNRVTPYGEKIYMTLSAYLELDHCIQP |
| | AVITKDVCMVFYSRDAKISPPRSLRSLFGSGYSKSPDSNRVTGIYELSLCKMSDTGSP |
| | GKMQRRRRKILDTSVAYVRGEENLAGWRPRGDSLILEHQWELEKLEKTRHFLLLRERL |
| | GDSIPKSLSDSLSPSLSSGTLSTSTSISSQISTTTFESAITPSESSGYDSGDIESLVD |
| | REKELATKCLQLLTHTFNREFSQVHGSVSDCKVSDISPIGRDPSESSFSSATLTPSST |
| | CPSLVDSRSNSLDQKTPEANSRASSPCPEFEQFQIVPAVETPYLARAGKNEFLNLVPD |
| | IEEIRSVVSKKGYLHFKEPLYSNWAKHFVVVRRPYVFIYNSDKDPVERGIINLSTAQV |
| | EYSEDQQAMVKTPNTFAVCTKHRCVLLQALNDKDMNDWLYAFNPLLAGTIRRSKLSRR |
| | CPSQSKY |

Further analysis of the NOV31a protein yielded the following properties shown in Table 31B.

TABLE 31B
Protein Sequence Properties NOV31a

| | |
|---|---|
| PSort | 0.5985 probability located in mitochondrial matrix space; |
| analysis: | 0.4900 probability located in nucleus; 0.3052 probability |
| | located in mitochondrial inner membrane; 0.3052 probability |
| | located in mitochondrial intermembrane space |
| SignalP | No Known Signal Sequence Predicted |
| analysis: | |

A search of the NOV31a protein against the Geneseq database, a proprietary database that contains sequences published in patents and patent publication, yielded several homologous proteins shown in Table 31C.

TABLE 31C
Geneseq Results for NOV31a

| | | | Identities/ | |
|---|---|---|---|---|
| | | | Similarities for | |
| Geneseq | Protein/Organism/Length | NOV31a Residues/ | the Matched | Expect |
| Identifier | [Patent #, Date] | Match Residues | Region | Value |
| AAB36227 | Human kinesin-like protein HKLP SEQ ID | 1 . . . 1805 | 1797/1821 (98%) | 0.0 |
| | NO: 4 - Homo sapiens, 1816 aa. | 1 . . . 1816 | 1800/1821 (98%) | |
| | [WO200063375-A1, 26 OCT. 2000] | | | |
| ABB07867 | Human kinesin-associated protein | 1 . . . 1804 | 1785/1820 (98%) | 0.0 |
| | having motor domain - Homo sapiens, | 1 . . . 1816 | 1790/1820 (98%) | |
| | 1823 aa. [WO200226965-A1, 04 APR. 2002] | | | |
| ABB07866 | Human kinesin-associated protein | 430 . . . 1805 | 1370/1385 (98%) | 0.0 |
| | lacking motor domain - Homo sapiens, | 1 . . . 1381 | 1372/1385 (98%) | |
| | 1381 aa. [WO200226965-A1, 04 APR. 2002] | | | |
| AAU28137 | Novel human secretory protein, Seq ID | 430 . . . 1805 | 1370/1385 (98%) | 0.0 |
| | No 306 - Homo sapiens, 1381 aa. | 1 . . . 1381 | 1372/1385 (98%) | |
| | [WO200166689-A2, 13 SEP. 2001] | | | |
| AAU28325 | Novel human secretory protein, Seq ID | 439 . . . 1805 | 1355/1376 (98%) | 0.0 |
| | No 682 - Homo sapiens, 1374 aa. | 3 . . . 1374 | 1360/1376 (98%) | |
| | [WO200166689-A2, 13 SEP. 2001] | | | |

In a BLAST search of public sequence datbases, the NOV31a protein was found to have homology to the proteins shown in the BLASTP data in Table 31D.

TABLE 31D
Public BLASTP Results for NOV31a

| | | NOV31a | Identities/ | |
|---|---|---|---|---|
| Protein | | Residues/ | Similarities | |
| Accession | | Match | for the | Expect |
| Number | Protein/Organism/Length | Residues | Matched Portion | Value |
| O60333 | Kinesin-like protein KIF1B | 1 . . . 1805 | 1783/1821 (97%) | 0.0 |
| | (Klp) - Homo sapiens (Human), | 1 . . . 1816 | 1791/1821 (97%) | |
| | 1816 aa. | | | |
| Q60575 | Kinesin-like protein KIF1B - | 1 . . . 1805 | 1745/1821 (95%) | 0.0 |
| | Mus musculus (Mouse), 1816 | 1 . . . 1816 | 1783/1821 (97%) | |
| | aa. | | | |
| Q8R524 | Kinesin-family protein 1Bp204 - | 1 . . . 1805 | 1741/1821 (95%) | 0.0 |
| | Rattus norvegicus (Rat), 1816 | 1 . . . 1816 | 1779/1821 (97%) | |
| | aa. | | | |
| Q96Q94 | Kinesin-related protein - Homo | 430 . . . 1804 | 1359/1384 (98%) | 0.0 |
| | sapiens (Human), 1388 aa. | 1 . . . 1381 | 1363/1384 (98%) | |
| O88658 | Kinesin-like protein KIF1B - | 1 . . . 700 | 657/704 (93%) | 0.0 |
| | Rattus norvegicus (Rat), 689 aa | 1 . . . 689 | 668/704 (94%) | |
| | (fragment). | | | |

PFam analysis predicts that the NOV31a protein contains the domains shown in the Table 31E.

TABLE 31E
Domain Analysis of NOV31a

| | | Identities/ | |
|---|---|---|---|
| | | Similarities | |
| Pfam | NOV31a | for the | Expect |
| Domain | Match Region | Matched Region | Value |
| kinesin | 11 . . . 378 | 183/418 (44%) | 6.7e−188 |
| | | 323/418 (77%) | |
| FHA | 550 . . . 621 | 22/85 (26%) | 1.6e−14 |
| | | 55/85 (65%) | |
| PH | 1690 . . . 1787 | 28/98 (29%) | 4.6e−18 |
| | | 78/98 (80%) | |

### Example 32

The NOV32 clone was analyzed, and the nucleotide and encoded polypeptide sequences are shown in Table 32A.

TABLE 32A
NOV32 Sequence Analysis

| | SEQ ID:317 | 3120 bp | |
|---|---|---|---|
| NOV32a, | GGAGGCCCGAGCGGCGCCCACCTGAGCCCCCGCGCTGGCGCCATGGCGGAGCAGGAGA | | |
| CG157477-01 | | | |
| DNA Sequence | GCCTGGAATTCGGCAAGGCAGACTTCGTGCTGATGGACACCGTCTCCATCCCCGAGTT | | |
| | CATGGCCAACCTCAGGCTCAGATTTGAAAAAGGGCGCATCTATACGTTCATTGGAGAA | | |
| | GTCGTCGTTTCTGTGAACCCTTACAAGTTGTTGAACATCTATGGAAGAGACACAATTG | | |
| | AGCAGTATAAAGGCCGTGAGCTGTATGAGAGACCGCCTCACCTTTTTGCTATTGCGGA | | |
| | TGCTGCTTACAAGGCTATGAAGAGGCGATCAAAAGACACTTGTATTGTGATATCAGGG | | |
| | GAAAGTGGAGCTGGTAAAACGGAAGCCAGTAAGTACATTATGCAGTATATTGCGGCCA | | |
| | TCACCAACCCCAGTCAGAGAGCAGAGGTTGAAAGAGTGAAGAATATGTTGCTTAAGTC | | |
| | CAACTGTGTTTTGGAAGCTTTTGGAAATGCCAAAACCAACCGTAATGACAACTCAAGC | | |
| | AGGTTTGGAAAATACATGGATATCAACTTTGACTTCAAGGGTGACCCTATTGGTGGGC | | |
| | ATATCAATAACTACTTACTAGAAAAGTCTCGAGTGATTGTGCAACAGCCAGGAGAAAG | | |
| | AAGCTTTCATTCTTTCTATCAGCTACTCCAAGGAGGTTCAGAACAAATGCTACGCTCT | | |
| | CTACATCTCCAGAAATCCCTTTCATCCTACAACTATATTCATGTGGGAGCTCAATTAA | | |
| | AGTCTTCTATCAATGATGCTGCCGAATTCAGAGTTGTTGCTGATGCCATGAAAGTCAT | | |
| | TGGCTTCAAACCTGAGGAGATCCAAACAGTGTATAAGATTTTGGCTGCTATTCTGCAC | | |
| | TTGGGAAATTTAAAATTTGTAGTACATGGTGACACGCCTCTTATTGAGAATGGCAAAG | | |
| | TAGTATCTATCATAGCAGAATTGCTCTCTACTAAGACAGATATGGTTGAGAAAGCCCT | | |
| | TCTTTACCGGACTGTGGCCACAGGCCGTGACATCATTGACAAGCAGCACACAGAACAA | | |
| | GAGGCCAGCTACGGCAGAGACGCCTTTGCCAAGGCAATATATGAGCGCCTTTTTTGTT | | |
| | GGATCGTTACTCGCATCAATGATATTATTGAGGTCAAGAACTATGACACCACAATCCA | | |
| | TGGGAACAACACTGTTATTGGTGTCTTGGATATCTATGGCTTTGAAATCTTTGACAAC | | |
| | AACAGTTTTGAACAATTCTGTATCAATTACTGCAATGAGAAACTGCAGCAGCTATTTA | | |
| | TTCAGCTGGTTCTGAAGCAAGAACAAGAGGAATACCAGCGGGAAGGGATCCCCTGGAA | | |
| | ACATATTGACTACTTCAACAATCAGATCATTGTTGACCTCGTGGAGCAACAGCACAAA | | |
| | GGGATCATTGCAATCCTTGATGATGCTTGCATGAATGTCGGCAAAGTCACCGATGAAA | | |
| | TGTTTCTTCAAGCACTTAACAGTAAATTGGGCAAACACGCCCATTTTTCCAGCCGAAA | | |
| | GCTCTGTGCCTCAGACAAAATTCTGGAGTTTGATCGAGATTTTCGAATTCGACATTAT | | |
| | GCAGGCGATGTAGTCTATTCTGTCATTGGTTTTATTGACAAAAATAAAGATACTTTAT | | |
| | TTCAAGATTTCAAGCGCCTTATGTATAACAGTTCAAATCCTGTGCTCAAGAATATGTG | | |
| | GCCTGAAGGCAAACTGAGCATTACAGAGGTGACCAAGCGACCTCTGACTGCTGCTACC | | |
| | TTGTTTAAGAATTCTATGATTGCTCTAGTAGACAACCTTGCATCAAAGGAACCATATT | | |
| | ACGTTCGTTGCATCAAACCCAATGACAAGAAATCTCCACAGATATTTGATGATGAACG | | |
| | CTGCCGGCACCAAGTAGAATATCTTGGACTACTGGAAAATGTGAGAGTGCGTCGGGCA | | |
| | GGATTTGCCTTCCGCCAGACATACGAGAAGTTTCTTCACAGGTATAAGATGATCTCTG | | |
| | AATTCACCTGGCCCAACCATGACCTTCCTTCAGACAAAGAGGCTGTCAAGAAACTAAT | | |
| | TGAACGGTGTGGTTTTCAGGATGATGTAGCTTATGGGAAGACCAAAATTTTCATTCGA | | |
| | ACACCCCGAACATTGTTTACCTTGGAAGAACTCCGTGCCCAGATGCTCATAAGGATTG | | |
| | TCCTCTTTCTACAAAAGGTGTGGCGGGGCACCCTGGCCCGCATGCGGTACAAAAGAAC | | |
| | CAAGGCAGCTCTGACAATAATCAGGTACTACCGGCGCTACAAAGTGAAGTCGTACATC | | |
| | CACGAGGTGGCCAGACGCTTCCATGGCGTCAAGACCATGCGAGACTACGGGAAGCACG | | |
| | TGAAGTGGCCAAGCCCTCCTAAAGTTCTTCGCCGTTTTGAGGAGGCCCTGCAGACGAT | | |
| | TTTCAATAGATGGAGAGCATCCCAGCTCATCAAGAGCATTCCGGCCTCAGACCTGCCC | | |
| | CAGGTCAGGGCAAAGGTTGCAGCCGTGGAAATGTTGAAGGGTCAAAGGGCTGACCTCG | | |
| | GGCTCCAGAGGGCCTGGGAGGGCAACTATCTTGCTTCAAAGCCAGATACACCTCAGAC | | |
| | CTCAGGCACTTTTGTCCCTGTTGCTAATGAATTGAAACGGAAGGACAAATACATGAAT | | |
| | GTCCTCTTTTCCTGTCACGTCCGTAAGGTAAATCGATTTAGTAAGGTGGTGGACAGAG | | |
| | CAATTTTTGTCACTGACCGTCACCTGTATAAAATGGATCCCACTAAACAGTACAAGGT | | |
| | GATGAAGACTATCCCTCTATACAATTTGACTGGTCTGAGTGTCTCCAATGGAAAGGAC | | |
| | CAACTTGTAGTGTTCCATACGAAAGACAACAAAGACCTCATTGTCTGCCTCTTCAGCA | | |
| | AACAGCCAACCCATGAGAGTCGAATTGGAGAACTTGTTGGAGTGCTGGTGAATCATTT | | |
| | CAAGAGTGAGAAGCGCCACCTTCAAGTGAACGTCACCAACCCAGTACAGTGCAGCCTG | | |
| | CACGGGAAGAAGTGCACCGTCTCCGTGGAGACGCGGCTCAACCAGCCCCAGCCCGACT | | |
| | TCACCAAGAATCGCTCGGGCTTCATCCTCAGCGTGCCCGGGAACTGACGCCCCGCGGA | | |
| | GGCCTGGCCCGGAGCCCGGCCACACTCCGAGTCCTGGGTCCCAGTC | | |
| | ORF Start: ATG at 43 | | ORF Stop: TGA at 3061 |
| | SEQ ID NO:318 | 1006 aa | MW at 116201.0 kD |

| | |
|---|---|
| NOV32a, | MAEQESLEFGKADFVLMDTVSMPEFMANLRLRFEKGRIYTFIGEVVVSVNPYKLLNIY |
| CG157477-01 | |
| Protein Sequence | GRDTIEQYKGRELYERPPHLFATADAAYKAMKRRSKDTCIVISGESGAGKTEASKYIM |
| | QYIAAITNPSQRAEVERVKNMLLKSNCVLEAFGNAKTNRNDNSSRFGKYMDINFDFKG |
| | DPIGGHINNYLLEKSRVIVQQPGERSFHSFYQLLQGGSEQMLRSLHLQKSLSSYNYIH |
| | VGAQLKSSINDAAEFRVVADAMKVIGFKPEEIQTVYKILAAILHLGNLKFVVDGDTPL |
| | IENGKVVSIIAELLSTKTDMVEKALLYRTVATGRDIIDKQHTEQEASYGRDAFAKAIY |
| | ERLFCWIVTRINDIIEVKNYDTTIHGKNTVIGVLDIYGFEIFDNNSFEQFCINYCNEK |
| | LQQLFIQLVLKQEQEEYQREGIPWKHIDYFNNQIIVDLVEQQHKGIIAILDDACMNVG |
| | KVTDEMFLEALNSKLGKHAHFSSRKLCASDKILEFDRDFRIRHYAGDVVYSVIGFIDK |
| | NKDTLFQDFKRLMYNSSNPVLKNMWPEGKLSITEVTKRPLTAATLFKMSMIALVDNLA |
| | SKEPYYVRCIKPNDKKSPQIFDDERCRHQVEYLGLLENVRVRRAGFAFRQTYEKFLHR |
| | YKMISEFTWPNHDLPSDKEAVKKLIERCGFQDDVAYGKTKIFIRTPRTLFTLEELRAQ |
| | MLIRIVLFLQKVWRGTLARMRYKRTKAALTIIRYYRRYKVKSYIHEVARRFHCVKTMR |
| | DYGKHVKWPSPPKVLRRFEEALQTIFNRWRASQLIKSIPASDLPQVRAKVAAVEMLKG |
| | QRADLGLQRAWEGNYLASKPDTPQTSGTFVPVANELKRKDKYMNVLFSCHVRKVNRFS |
| | KVEDPAIFVTDRHLYKMDPTKQYKVMKTIPLYNLTGLSVSNGKDQLVVFHTKDNKDLI |
| | VCLFSKQPTHESRIGELVGVLVNHFKSEKRHLQVNVTNPVQCSLHGKKCTVSVETRLN |
| | QPQPDFTKNRSGFILSVPGN |

Further analysis of the NOV32a protein yielded the following properties shown in Table 32B.

TABLE 32B
Protein Sequence Properties NOV32a

| | |
|---|---|
| PSort | 0.7600 probability located in nucleus; 0.3760 probability |
| analysis: | located in microbody (peroxisome); 0.1000 probability |
| | located in mitochondrial matrix space; 0.1000 probability |
| | located in lysosome (lumen) |
| SignalP | No Known Signal Sequence Predicted |
| analysis: | |

A search of the NOV32a protein against the Geneseq database, a proprietary database that contains sequences published in patents and patent publication, yielded several homologous proteins shown in Table 32C.

TABLE 32C
Geneseq Results for NOV32a

| | | NOV32a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAM80123 | Human protein SEQ ID NO 3769 - | 243 . . . 1006 | 764/764 (100%) | 0.0 |
| | Homo sapiens, 764 aa. | 1 . . . 764 | 764/764 (100%) | |
| | [WO200157190-A2, 09 AUG. | | | |
| | 2001] | | | |
| AAM79139 | Human protein SEQ ID NO 1801 - | 254 . . . 1006 | 752/753 (99%) | 0.0 |
| | Homo sapiens, 753 aa. | 1 . . . 753 | 752/753 (99%) | |
| | [WO200157190-A2, 09 AUG. | | | |
| | 2001] | | | |
| ABG16605 | Novel human diagnostic protein | 333 . . . 1006 | 670/674 (99%) | 0.0 |
| | #16596 - Homo sapiens, 674 aa. | 1 . . . 674 | 671/674 (99%) | |
| | [WO200175067-A2, 11 OCT. | | | |
| | 2001] | | | |
| AAU23125 | Novel human enzyme polypeptide | 1 . . . 1004 | 611/1016 (60%) | 0.0 |
| | #211 - Homo sapiens, 1026 aa. | 9 . . . 1024 | 784/1016 (77%) | |
| | [WO200155301-A2, 02 AUG. | | | |
| | 2001] | | | |
| AAU23128 | Novel human enzyme polypeptide | 1 . . . 841 | 532/853 (62%) | 0.0 |
| | #214 - Homo sapiens, 909 aa. | 9 . . . 861 | 676/853 (78%) | |
| | [WO200155301-A2, 02 AUG. | | | |
| | 2001] | | | |

In a BLAST search of public sequence datbases, the NOV32a protein was found to have homology to the proteins shown in the BLASTP data in Table 32D.

TABLE 32D
Public BLASTP Results for NOV32a

| | | NOV32a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q63357 | Myosin I - Rattus norvegicus | 1 . . . 1006 | 985/1006 (97%) | 0.0 |
| | (Rat), 1006 aa. | 1 . . . 1006 | 998/1006 (98%) | |
| A53933 | myosin I myr 4 - rat, 1006 aa. | 1 . . . 1006 | 983/1006 (97%) | 0.0 |
| | | 1 . . . 1006 | 996/1006 (98%) | |
| O94832 | KIAA0727 protein - Homo sapiens | 333 . . . 1006 | 674/674 (100%) | 0.0 |
| | (Human), 674 aa (fragment). | 1 . . . 674 | 674/674 (100%) | |
| Q23978 | Myosin IA (MIA) (Brush border | 8 . . . 1004 | 542/1004 (53%) | 0.0 |
| | myosin IA) (BBMIA) - Drosophila | 6 . . . 1006 | 706/1004 (69%) | |
| | melanogaster (Fruit fly), 1011 aa. | | | |
| S45573 | myosin IA - fruit fly (Drosophila | 8 . . . 1004 | 541/1004 (53%) | 0.0 |
| | melanogaster), 1011 aa. | 6 . . . 1006 | 704/1004 (69%) | |

PFam analysis predicts that the NOV32a protein contains the domains shown in the Table 32E.

TABLE 32E
Domain Analysis of NOV32a

| | | Identities/ | |
|---|---|---|---|
| | | Similarities | |
| Pfam | NOV32a | for the | Expect |
| Domain | Match Region | Matched Region | Value |
| myosin_head | 13 . . . 682 | 314/743 (42%) | 0 |
| | | 544/743 (73%) | |
| IQ | 699 . . . 719 | 10/21 (48%) | 0.0053 |
| | | 16/21 (76%) | |

### Example 33

The NOV33 clone was analyzed, and the nucleotide and encoded polypeptide sequences are shown in Table 33A.

TABLE 33A
NOV33 Sequence Analysis

| | SEQ ID NO:319 | 3921 bp | |
|---|---|---|---|
| NOV33a, | CAGAAGTTGCGCGCAGGCCGGCGGGCGGGAGCGGACACCGAGGCCGGCGTGCAGGCGT | | |
| CG157486-01 | | | |
| DNA Sequence | GCGGGTGTGCGGGAGCCGGGCTCGGGGGGATCGGACCGAGAGCGAGAAGCGCGGCATG | | |
| | GAGCTCCAGGCAGCCCGCGCCTGCTTCGCCCTGCTGTGGGGCTGTGCGCTGGCCGCGG | | |
| | CCGCGGCGGCGCAGGGCAAGGAAGTGGTACTGCTGGACTTTGCTGCAGCTGGAGGGGA | | |
| | GCTCGGCTGGCTCACACACCCGTATGGCAAAGGGTGGGACCTGATGCAGAACATCATG | | |
| | AATGACATGCCGATCTACATGTACTCCGTGTGCAACGTGATGTCTGGCGACCAGGACA | | |
| | ACTGGCTCCGCACCAACTGGGTGTACCGAGGAGAGGCTGAGCGTATCTTCATTGAGCT | | |
| | CAAGTTTACTGTACGTGACTGCAACAGCTTCCCTGGTGCCGCCAGCTCCTGCAAGGAG | | |
| | ACTTTCAACCTCTACTATGCCGAGTCGGACCTGGACTACGGCACCAACTTCCAGAAGC | | |
| | GCCTGTTCACCAAGATTGACACCATTGCGCCCGATGAGATCACCGTCAGCAGCGACTT | | |
| | CGAGGCACGCCACGTGAAGCTGAACGTGGAGGAGCGCTCCGTGGGGCCGCTCACCCGC | | |
| | AAAGGCTTCTACCTGGCCTTCCAGGATATCGGTGCCTGTGTGGCGCTGCTCTCCGTCC | | |
| | GTGTCTACTACAAGAAGTGCCCCGAGCTGCTGCAGGGCCTGGCCCACTTCCCTGAGAC | | |
| | CATCGCCGGCTCTGATGCACCTTCCCTGGCCACTGTGGCCGGCACCTGTGTGGACCAT | | |
| | GCCGTGGTGCCACCGGCGGGTGAAGAGCCCCGTATGCACTGTGCAGTGGATGGCGAGT | | |
| | GGCTGGTGCCCATTGGGCAGTGCCTGTGCCAGGCAGGCTACGAGAAGGTGGAGGATGC | | |
| | CTGCCAGGCCTGCTCGCCTGGATTTTTTAAGTTTGAGGCATCTGAGAGCCCCTGCTTG | | |
| | GAGTGCCCTGAGCACACGCTGCCATCCCCTGAGGGTGCCACCTCCTGCGAGTGTGAGG | | |
| | AAGGCTTCTTCCGGGCACCTCAGGACCCAGCGTCGATGCCTTGCACACGACCCCCCTC | | |
| | CGCCCCACACTACCTCACAGCCGTGGGCATGGGTGCCAAGGTGGAGCTGCGCTGGACG | | |
| | CCCCCTCAGGACAGCGGGGGCCGCGAGGACATTGTCTACAGCGTCACCTGCGAACAGT | | |
| | GCTGGCCCGAGTCTGGGGAATGCGGGCCGTGTGAGGCCAGTGTGCGCTACTCGGAGCC | | |
| | TCCTCACGCACTGACCCGCACCAGTGTGACAGTGAGCGACCTGGAGCCCCACATGAAC | | |
| | TACACCTTCACCGTGGAGGCCCGCAATGGCGTCTCAGGCCTGGTAACCAGCCGCAGCT | | |
| | TCCGTACTGCCAGTGTCAGCATCAACCAGACAGAGCCCCCCAAGGTGAGGCTGGAGGG | | |
| | CCGCAGCACCACCTCGCTTAGCGTCTCCTGGAGCATCCCCCCGCCGCAGCAGAGCCGA | | |
| | GTGTGGAAGTACGAGGTCACTTACCGCAAGAAGGGAGACTCCAACAGCTACAATGTGC | | |
| | GCCGCACCGAGGGTTTCTCCGTGACCCTGGACGACCTGGCCCCAGACACCACCTACCT | | |
| | GGTCCAGGTGCAGGCACTGACGCAGGAGGGCCAGGGGGCCGGCAGCAAGGTGCACGAA | | |
| | TTCCAGACGCTGTCCCCGGAGGGATCTGGCAACTTGGCGGTGATTGGCGGCGTGGCTG | | |
| | TCGGTGTGGTCCTGCTTCTGGTGCTGGCAGGAGTTGGCTTCTTTATCCACCCCAGGAG | | |
| | GAAGAACCAGCGTGCCCGCCAGTCCCCGGAGGACGTTTACTTCTCCAAGTCAGAACAA | | |
| | CTGAAGCCCCTGAAGACATACGTGGACCCCCACACATATGAGGACCCCAACCAGGCTG | | |
| | TGTTGAAGTTCACTACCGAGATCCATCCATCCTGTGTCACTCGGCAGAAGGTGATCGG | | |
| | AGCAGGAGAGTTTGGGGAGGTGTACAAGGGCATGCTGAAGACATCCTCGGGGAAGAAG | | |
| | GAGGTGCCGGTGGCCATCAAGACGCTGAAAGCCGGCTACACAGAGAAGCAGCGAGTGG | | |
| | ACTTCCTCGGCGAGGCCGGCATCATGGGCCAGTTCAGCCACCACAACATCATCCGCCT | | |
| | AGAGGGCGTCATCTCCAAATACAAGCCCATGATGATCATCACTGAGTACATGGAGAAT | | |
| | GGGGCCCTGGACAAGTTCCTTCGGGAGAAGGATGGCGAGTTCAGCGTGCTGCAGCTGG | | |
| | TGGGCATGCTGCGGGGCATCGCAGCTGGCATGAAGTACCTGGCCAACATGAACTATGT | | |
| | GCACCGTGACCTGGCTGCCCGCAACATCCTCGTCAACAGCAACCTGGTCTGCAAGGTG | | |
| | TCTGACTTTGGCCTGTCCCGCGTGCTGGAGGACGACCCCGAGGCCACCTACACCACCA | | |
| | GTGGCGGCAAGATCCCCATCCGCTGGACCGCCCCGGAGGCCATTTCCTACCGGAAGTT | | |
| | CACCTCTGCCAGCGACGTGTGGAGCTTTGGCATTGTCATGTGGGAGGTGATGACCTAT | | |
| | GGCGAGCGGCCCTACTGGGAGTTGTCCAACCACGAGGTGATGAAAGCCATCAATGATG | | |
| | GCTTCCGGCTCCCCACACCCATGGACTGCCCCTCCGCCATCTACCAGCTCATGATGCA | | |
| | GTGCTGGCAGCAGGAGCGTGCCCGCCGCCCCAAGTTCGCTGACATCGTCAGCATCCTG | | |
| | GACAAGCTCATTCGTGCCCCTGACTCCCTCAAGACCCTGGCTGACTTTGACCCCCGCG | | |
| | TGTCTATCCGGCTCCCCAGCACGAGCGGCTCGGAGGGGGTGCCCTTCCGCACGGTGTC | | |
| | CGAGTGGCTGGAGTCCATCAAGATGCAGCAGTATACGGAGCACTTCATGGCGGCCGGC | | |
| | TACACTGCCATCGAGAAGGTGGTGCAGATGACCAACGACGACATCAAGAGGATTGGGG | | |
| | TGCGGCTGCCCGGCCACCAGAAGCGCATCGCCTACAGCCTGCTGGGACTCAAGGACCA | | |
| | GGTGAACACTGTGGGGATCCCCATCTGAGCCTCGACAGGGCCTGGAGCCCCATCGGCC | | |
| | AAGAATACTTGAAGAAACAGAGTGGCCTCCCTGCTGTGCCATGCTGGGCCACTGGGGA | | |
| | CTTTATTTATTTCTAGTTCTTTCCTCCCCCTGCAACTTCCGCTGAGGGGTCTCGGATG | | |
| | ACACCCTGGCCTGAACTGAGGAGATGACCAGGGATGCTGGGCTGGGCCCTCTTTCCCT | | |
| | GCGAGACGCACACAGCTGAGCACTTAGCAGGCACCGCCACGTCCCAGCATCCCTGGAG | | |
| | CAGGAGCCCCGCCACAGCCTTCGGACAGACATATGGGATATTCCCAAGCCGACCTTCC | | |
| | CTCCGCCTTCTCCCACATGAGGCCATCTCAGGAGATGGAGGGCTTGGCCCAGCGCCAA | | |
| | GTAAACAGGGTACCTCAAGCCCCATTTCCTCACACTAAGAGGGCAGACTGTGAACTTG | | |
| | ACTGGGTGAGACCCAAAGCGGTCCCTGTCCCTCTAGTGCCTTCTTTAGACCCTCGGGC | | |
| | CCCATCCTCATCCCTGACTGGCCAAACCCTTGCTTTCCTGGGCCTTTGCAAGATGCTT | | |
| | GGTTGTGTTGAGGTTTTTAAATATATATTTTGTACTTTGTGGAGAGAATGTGTGTGTG | | |
| | TGGCAGGGGGCCCCGCCAGGGCTGGGGACAGAGGGTGTCAAACATTCGTGAGCTGGGG | | |
| | ACTCAGGGACCGGTGCTGCAGGAGTGTCCTGCCCATGCCCCAGTCGGCCCCATCTCTC | | |
| | ATCCTTTTGGATAAGTTTCTATTCTGTCAGTGTTAAAGATTTTGTTTTGTTGGACATT | | |
| | TTTTTCGAATCTTAATTTATTATTTTTTTTATATTTATTGTTAGAAAATGACTTATTT | | |
| | CTGCTCTGGAATAAAGTTGCAGATGATTCAAACCG | | |
| | ORF Start: ATG at 114 | | ORF Stop: TGA at 3042 |
| | SEQ ID NO:320 | 976 aa | MW at 108265.3 kD |

| | |
|---|---|
| NOV33a, | MELQAARACFALLWGCALAAAAAAQGKEVVLLDFAAAGGELGWLTHPYGKGWDLMQNI |
| CG157486-01 | |
| Protein Sequence | MNDMPIYMYSVCNVMSGDQDNWLRTNWVYRGEAERIFIELKFTVRDCNSFPGGASSCK |
| | ETFNLYYAESDLDYGTNFQKRLFTKIDTIAPDEITVSSDFEARHVKLNVEERSVGPLT |
| | RKGFYLAFQDIGACVALLSVRVYYKKCPELLQGLAHFPETIAGSDAPSLATVAGTCVD |
| | HAVVPPGGEEPRNHCAVDGEWLVPIGQCLCQAGYEKVEDACQACSPGFFKFEASESPC |
| | LECPEHTLPSPEGATSCECEEGFFRAPQDPASMPCTRPPSAPHYLTAVGMGAKVELRW |
| | TPPQDSGGREDIVYSVTCEQCWPESGECGPCEASVRYSEPPHGLTRTSVTVSDLEPHM |
| | NYTFTVEARNGVSGLVTSRSFRTASVSINQTEPPKVRLEGRSTTSLSVSWSIPPPQQS |
| | RVWKYEVTYRKKGDSNSYNVRRTEGFSVTLDDLAPDTTYLVQVQALTQEGQGAGSKVH |
| | EFQTLSPEGSGNLAVIGGVAVGVVLLLVLAGVGFFIHRRRKNQRARQSPEDVYFSKSE |
| | QLKPLKTYVDPHTYEDPNQAVLKFTTEIHPSCVTRQKVIGAGEFGEVYKGMLKTSSGK |
| | KEVPVAIKTLKAGYTEKQRVDFLGEAGIMGQFSHHNIIRLEGVISKYKPMMIITEYME |
| | NGALDKFLREKDGEFSVLQLVGMLRGIAAGMKYLANMNYVHRDLAARNILVNSNLVCK |
| | VSDFGLSRVLEDDPEATYTTSGGKIPIRWTAPEAISYRKFTSASDVWSFGIVMWEVMT |
| | YGERPYWELSNHEVMKAINDGFRLPTPMDCPSAIYQLMMQCWQQERARRPKFADIVSI |
| | LDKLIRAPDSLKTLADFDPRVSIRLPSTSGSEGVPFRTVSEWLESIKMQQYTEHFMAA |
| | GYTAIEKVVQMTNDDIKRIGVRLPGHQKRIAYSLLGLKDQVNTVGIPI |

Further analysis of the NOV33a protein yielded the following properties shown in Table 33B.

TABLE 33B
Protein Sequence Properties NOV33a

| | |
|---|---|
| PSort | 0.4600 probability located in plasma membrane; 0.1000 |
| analysis: | probability located in endoplasmic reticulum (membrane); |
| | 0.1000 probability located in endoplasmic reticulum |
| | (lumen); 0.1000 probability located in outside |
| SignalP | Cleavage site between residues 24 and 25 |
| analysis: | |

A search of the NOV33a protein against the Geneseq database, a proprietary database that contains sequences published in patents and patent publication, yielded several homologous proteins shown in Table 33C.

TABLE 33C
Geneseq Results for NOV33a

| | | NOV33a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAR85090 | EPH-like receptor protein tyrosine | 11 . . . 976 | 524/984 (53%) | 0.0 |
| | kinase HEK7 - Homo sapiens, 991 | 14 . . . 991 | 680/984 (68%) | |
| | aa. [WO9528484-A1, 26 OCT. | | | |
| | 1995] | | | |
| AAR85092 | EPH-like receptor protein tyrosine | 13 . . . 969 | 504/979 (51%) | 0.0 |
| | kinase HEK11 - Homo sapiens, 998 | 16 . . . 988 | 659/979 (66%) | |
| | aa. [WO9528484-A1, 26 OCT. | | | |
| | 1995] | | | |
| AAW03421 | Mouse developmental kinase 1 - | 9 . . . 969 | 505/982 (51%) | 0.0 |
| | Mus sp, 998 aa. [WO9621013-A1, | 14 . . . 988 | 660/982 (66%) | |
| | 11 JUL. 1996] | | | |
| AAW83147 | Rat receptor tyrosine kinase Ehk-1 - | 13 . . . 940 | 503/969 (51%) | 0.0 |
| | Rattus sp, 1005 aa. [US5843749-A, | 42 . . . 1003 | 654/969 (66%) | |
| | 01 DEC. 1998] | | | |
| AAB08665 | Amino acid sequence of a human | 28 . . . 976 | 499/964 (51%) | 0.0 |
| | EphA3 HLA class II-binding | 29 . . . 983 | 652/964 (66%) | |
| | peptide - Homo sapiens, 983 aa. | | | |
| | [WO200050589-A1, 31 AUG. | | | |
| | 2000] | | | |

In a BLAST search of public sequence datbases, the NOV33a protein was found to have homology to the proteins shown in the BLASTP data in Table 33D.

TABLE 33D
Public BLASTP Results for NOV33a

| | | NOV33a | Identities/ | |
|---|---|---|---|---|
| Protein | | Residues/ | Similarities | |
| Accession | | Match | for the | Expect |
| Number | Protein/Organism/Length | Residues | Matched Portion | Value |
| AAH37166 | EphA2 - Homo sapiens (Human), | 1 . . . 976 | 976/976 (100%) | 0.0 |
| | 976 aa. | 1 . . . 976 | 976/976 (100%) | |
| P29317 | Ephrin type-A receptor 2 precursor | 1 . . . 976 | 972/976 (99%) | 0.0 |
| | (EC 2.7.1.112) (Tyrosine-protein | 1 . . . 976 | 972/976 (99%) | |
| | kinase receptor ECK) (Epithelial | | | |
| | cell kinase) - Homo sapiens | | | |
| | (Human), 976 aa. | | | |
| Q03145 | Ephrin type-A receptor 2 precursor | 1 . . . 976 | 905/978 (92%) | 0.0 |
| | (EC 2.7.1.112) (Tyrosine-protein | 1 . . . 977 | 931/978 (94%) | |
| | kinase receptor ECK) (Epithelial | | | |
| | cell kinase) (MPK-5) (SEK-2) - | | | |
| | Mus musculus (Mouse), 977 aa. | | | |
| I48974 | receptor-protein tyrosine kinase - | 1 . . . 976 | 886/978 (90%) | 0.0 |
| | mouse, 975 aa. | 1 . . . 975 | 916/978 (93%) | |
| Q9PWR5 | Eph receptor tyrosine kinase | 25 . . . 976 | 690/957 (72%) | 0.0 |
| | precursor - Xenopus laevis (African | 24 . . . 977 | 798/957 (83%) | |
| | clawed frog), 977 aa. | | | |

PFam analysis predicts that the NOV33a protein contains the domains shown in the Table 33E.

TABLE 33E
Domain Analysis of NOV33a

| | | Identities/ | |
|---|---|---|---|
| | | Similarities | |
| Pfam | NOV33a | for the | Expect |
| Domain | Match Region | Matched Region | Value |
| EPH_lbd | 28 . . . 201 | 103/178 (58%) | 2.4e−126 |
| | | 167/178 (94%) | |
| fn3 | 329 . . . 424 | 29/98 (30%) | 4.1e−12 |
| | | 72/98 (73%) | |
| fn3 | 436 . . . 519 | 32/87 (37%) | 2.3e−20 |
| | | 67/87 (77%) | |
| pkinase | 613 . . . 868 | 82/292 (28%) | 1.7e−75 |
| | | 204/292 (70%) | |
| SAM | 902 . . . 966 | 30/68 (44%) | 7.1e−26 |
| | | 58/68 (85%) | |

### Example 34

The NOV34 clone was analyzed, and the nucleotide and encoded polypeptide sequences are shown in Table 34A.

TABLE 34A
NOV34 Sequence Analysis

| | SEQ ID NO:321 | 14399 bp | |
|---|---|---|---|
| NOV34a, | ATGGCGAACGTGCAGGTCGCCGTGCGGGTCCGGCCGCTCAGCAAGAGGGAGACCAAAG | | |
| CG157505-01 | | | |
| DNA Sequence | AAGGGGGAAGAATTATTGTGGAAGTTGATGGCAAAGTGGCAAAAATCAGGAATTTAAA | | |
| | GGTAGACAATCGACCAGATGGCTTTGGGGACTCCCGGGAGAAGGTTATGGCATTTGGC | | |
| | TTTGATTACTGCTACTGGTCAGTCAACCCAGAGCATCCCCAGTATGCATCTCAAGATG | | |
| | TGGTATTCCAGGATTTAGGGATGGAAGTACTGTCTGGAGTTGCCAAAGGCTATAACAT | | |
| | ATGCCTTTTTGCTTATGGACAGACAGGCTCTGGGAAGACATATACCATGCTGGGGACC | | |
| | CCAGCCTCTGTTGGGTTGACACCACGGATATGTGAGGGTCTCTTCGTCAGGGAGAAAG | | |
| | ACTGTGCCTCACTGCCTTCCTCCTGTAGGATAAAAGTAAGTTTTCTAGAAATCTATAA | | |
| | TGAACGGGTGCGGGATCTGTTGAAGCAATCTGGTCAAAAAAAGTCCTATACCCTGCGG | | |
| | GTCAGGGAGCATCCAGAGATGGGGCCCTATGTACAAGGTTTATCTCAACATGTAGTTA | | |
| | CCAATTATAAGCAAGTAATCCAACTCTTGGAGGAGGGAATTGCAAACAGGATCACAGC | | |
| | AGCCACCCATGTTCATGAGGCCAGCAGCAGATCCCACGCCATTTTCACGATCCACTAC | | |
| | ACGCAGGCAATCCTGGAGAACAACCTCCCTTCTGAAATGGCTAGCAAGATCAACCTTG | | |
| | TGGACCTAGCAGGCAGCGAAAGAGCAGATCCCAGTTACTGTAAGGACCGCATTGCTGA | | |
| | AGGAGCCAATATCAACAAGTCCCTTGTGACTCTAGGAATTGTCATCTCCACCTTAGCC | | |
| | CAGAACTCCCAAGTTTTCAGCAGCTGCCAGAGCCTCAACAGCTCAGTCAGCAATGGTG | | |
| | GTGACAGTGGGATCCTTAGCTCTCCTTCTGGGACCAGCAGTGGAGGGGCACCCTCCCG | | |
| | AAGGCAGTCTTATATCCCATACCGAGACTCTGTGTTGACCTGGCTGCTGAAGGACAGC | | |
| | CTTGGACGCAACTCTAAAACCATCATGGTTGCCAGTGTGTCTCCTGCACACACTAGCT | | |
| | ACAGTGAGACCATGAGCACACTGAGATATGCATCCAGTGCCAAAAACATTATCAACAA | | |
| | GCCACGAGTAAATGAGGATGCAAACTTAAAACTGATTAGAGAACTCAGAGAAGAGATT | | |
| | GAAAGACTGAAAGCCCTGCTGCTGAGCTTTGAACTGAGAAACTTCAGTTCATTGAGTG | | |
| | ATGAAAACCTGAAGGAGCTGGTTCTCCAAAATGAATTGAAGATAGACCAGCTGACTAA | | |
| | AGACTGGACCCAGAAGTGGAATGATTGGCAGGCCCTCATGGAGCATTACAGTGTGGAC | | |
| | ATCAACAGGAGGAGGGCTCGGGTGGTCATCGACTCCAGCCTGCCACACTTGATGGCCT | | |
| | TGGAGGATGATGTGCTCAGCACAGGTGTTCTGCTCTATCATCTCAAGGAAGGGACAAC | | |
| | AAAAATAGGAAGGATTGACTCAGACCAGGAACAGGACATTGTCCTGCAGGGTCAGTGG | | |
| | ATTGAGAGAGACCACTGCACTATCACCAGTGCCTGTGGTGTAGTTGTTCTACGACCTG | | |
| | CCCGTGGGGCCCGCTGTACAGTCAATGGCCGGGAGGTCACTGCCTCCTGCCGTCTGAC | | |
| | TCAAGGAGCTGTCATAACCCTGGGGAAGGCACAGAAGTTCCGATTCAACCACCCAGCA | | |
| | GAGGCTGCTGTCCTGCGGCAGCGAAGGCAGGTTGGAGAGGCTGCTGCTGGTCGTGGCT | | |
| | CGTTGGAGTGGCTGGATTTGGATGGAGATCTCGCTGCCTCCCGGCTGGGTCTCTCCCC | | |
| | TTTGCTTTGGAAGGAAAGGAGAGCGCTTGAAGAGCAATGTGACGAGGACCATCAGACA | | |
| | CCGAGGGATGGAGAGACATCCCACAGGGCCCAGATTCAGCAGCAGCAGAGCTACGTAG | | |
| | AGGATTTGAGGCATCAAATCCTAGCAGAAGAGATTCGAGCTGCGAAGGAACTGGAATT | | |
| | TGACCAAGCTTGGATTAGCCAGCAGATTAAAGAAAACCAGCAGTGTCTGCTCAGAGAA | | |
| | GAGACCTGCCTGGCCAGCTTGCAACAGCAGCAGCAAGAAGACCAGGTAGCAGAGAAAG | | |
| | AACTTGAGGCATCTGTGGCACTTGATGCTTGGCTTCAGACAGATCCTGAGATTCAGCC | | |
| | ATCCCCATTTGTCCAAAGTCAGAAAAGGGTGGTGCACCTGCAGCTCCTGCGGAGACAC | | |
| | ACTCTTCGGGCAGCAGAGCGGAATGTCCGGCGGAAAAAGGTCTCATTCCAGCTAGAGA | | |
| | GAATCATCAAAAAGCAGAGGCTGCTGGAGGCCCAGAAGAGACTGGAGAAGCTCACGAC | | |
| | ATTGTGCTGGCTCCAGGATGACAGCACCCAGGAGCCCCCATACCAGGTCCTCAGCCCT | | |
| | GATGCCACAGTCCCACGGCCTCCATGTAGAAGCAAATTGACGAGTTGCAGTTCTTTGA | | |
| | GCCCCCAAAGACTCTGCAGCAAGCACATGCCCCAGCTACACAGCATTTTCCTAAGTTG | | |
| | GGATCCCTCTACCACATTGCCACCTAGGCCTGACCCTACACACCAAACATCAGAGAAA | | |
| | ACATCATCAGAAGAGCATTTGCCACAGGCTGCTTCCTACCCTGCAAGGACAGGGTGCC | | |
| | TCCGCAAGAACGGCCTGCATTCCTCAGGTCATGGGCAGCCCTGCACAGCCAGAGCAGC | | |
| | CTTGGCCAGGAAGGCAGCCTCAGCTCCAGACCCTTGCCTCACCATGAGTCCCAACTCT | | |
| | GTTGGCATCCAGGAAATGGAGATGGGGGTTAAGCAGCCCCATCAGATGGTGAGCCAGG | | |
| | GCTTAGCATCTCTGAGGAAATCAGCTAACAAACTAAAGCCAAGGCATGAGCCAAAGAT | | |
| | CTTCACCTCTACTACCCAGACCAGAGGGGCGAAGGGACTAGCAGACCCTAGCCACACA | | |
| | CAAGCTGGGTGGCGAAAAGAAGGGAACCTTGGGACCCACAAGGCTGCTAAGGGAGCCA | | |
| | GTTGCAATTCCTTGTATCCTCATGGACCCAGGCAGACTGCTGGGCACGGAAAGGCAGT | | |
| | CAAGACTTTTTGGACAGAATACAAACCACCTTCTCCAAGCAGGGCATCAAAAAGGCAT | | |
| | CAGAGGGTTCTGGCAACTAGGGTCAGAAATATTACCAAAAAGTCCTCTCACTTGCCTC | | |
| | TTGGCAGTCCTTTGAAGAGACAACAAAATACAAGGGACCCAGACACCATGGTCCCACT | | |
| | CACAGATTTCACCCCAGTAATGGATCATTCAAGAGAAAAAGACAATGATTTATCTGAC | | |
| | ACAGATAGCAACTACTCATTGGATTCTCTCTCATGTGTCTATGCCAAAGCCCTGATAG | | |
| | AGCCACTGAAGCCAGAGGAGAGGAAATGGGATTTCCCAGAGCCAGAGAACTCTGAAAG | | |
| | TGATGACAGCCAACTATCTGAGGACTCACTGGCTGAGAAGAGGTACCAAAGCCCCAAA | | |
| | AACAGGCTAGGGGGCAATCGTCCCACCAACAACCGTGGCCAACCCAGGACCAGAACTA | | |
| | GAGCTTCTGTGAGGGGCTTCACTGCAGCCTCAGACAGTGACCTACTTGCTCAAACTCA | | |
| | TAGGAGCTTCTCCTTGGATAGCCTGATTGATGCAGAGGAAGAACTGGGGGAAGATCAG | | |
| | CAAGAAGAACCTTTCCCTGGTTCAGCTGACGAGATACCCACAGAGACTTTTTGGCACC | | |
| | TGGAGGACTCTAGTCTGCCTGTAATGGACCAAGAGGCAATATGCAGGCTTGGTCCCAT | | |
| | CAACTACAGAACAGCAGCTAGGCTGGATGCCGTCCTGCCAATGAGCAGTTCGTTTTAC | | |
| | CTTGATCCTCAGTTCCAACCCCATTGTGAGCTCCAACCCCATTGTGAGCTCCAACCCC | | |
| | ATTGTGAGCTCCAGCCCCATTGTGAGCAGGCTGAATCACAGGTAGAGCCAAGCTACTC | | |
| | TGAACAAGCCGACTCTCTCCAAGGCATGCAGCTTTCAAGAGAGAGCCCACTGATGTCC | | |
| | ATGGATTCCTGGTTTTCCTGTGACTCTAAGATCAACCCCAGCAGCCCCCCAGGAATAG | | |
| | TGGGTTCTTTATGTCCAAGTCCTGATATGCAGGAATTTCACTCCTGTAAGGGGGAGAG | | |
| | GCCTGGATACTGGCCAAATACTGAGGAACTAAAGCCATCAGATGCAGAAACGGTTCTG | | |
| | CCATATAGCTCCAAACTGCACCAAGGCAGTACTGAGCTCCTCTGCAGTGCAAGAGATG | | |
| | AGCACACAGCCTCTGCTGCTGATACGTCTAGGCTGTCTCTCTGGGGAATTCAAAGGCT | | |
| | TATTCAACCAGGAGCTGATGGCACCTTTCAGGGCAGATGTATCCCTGACATGACCCAG | | |
| | CAGGGCAGCTCTGAAGCATCCCACAATTCTAGCGTATCAAACGTGCTGGCTGCCTCTG | | |
| | CCACCACCTTGACTCATGTAGGCAGCACCCATGAAAGGGATTGGTCTGCCCTTCAGCA | | |
| | GAAGTACCTCCTTGAACTCTCTTGTCCTGTTTTGGAGGCCATAGGAGCACCCAAGCCA | | |
| | GCTTACCCCTACCTTGAGGAAGACTCTGGTTCCCTGGCCCAACCTTCTAGCAAAGGAG | | |
| | GAGATACTCTATTGCCAGTTGGCCCTAGGGTATCTAGCAATCTGAATCTCAACAACTT | | |
| | TCCAGTCCATCTGTCCAGAATCAGGCGTTTGAGGGCAGAGAAAGAACAGCACAGTTTA | | |
| | AATGCCAAATTAGAAGGTGTTTCAGATTTCTTTAGCACTAGTGAGAAAGAGGCGAGTT | | |
| | ATGACGAAACTTATTCGGCAGACTTAGAATCATTGTCTGCTTCTCGATCTACAAATGC | | |
| | ACAGGTCTTTGCAACAGAGAACGCGATACCAGATTCCATGACAGAAGCATGTGAAGTC | | |
| | AAGCAGAACAACTTGGAAGAATGCCTTCAGAGTTGCAGGAAACCTGGACTGATGACTT | | |
| | CCTCTGATGAGGATTTTTTCCAGAAGAACGCTTGTCACAGTAATGTCACTACAGCCAC | | |
| | CAAAGCAGACCATTGGTCCCAAGGCTGGGCTCCTCTCAGGAAAAATAGTGCAGTCCAG | | |
| | CCAGGGCAATTAAGTCCCGACAGCCACTACCCACTAGAGGAAGAGAAGACAGATTGCC | | |
| | AGGAGAGCTCTAAGGAAGCAGTTAGAACACACATAAATGTTTCCTTTGCCCTTCCTTC | | |
| | AGGTCCAGAGCTATACCTTCACTCTGCTCCCTGGAATCCATTGTCATCTTCCCTGCAG | | |
| | CCCCCACTCTTGGAAACATTCTATGTGACCAAAAGCAGGGATGCCCTGACAGAAACTG | | |
| | CCTTAGAGATTCCAGCTTGCAGAGAAGTAAGGGTACCCTCCCCACCCCCCAGGGAAGC | | |
| | CTGGGGCTTTGGTCACAACCACCAAGCTCTCCAAGGTGCTTATTTGAAGAATAATTTG | | |
| | CCAGTGCTGTTACAAAACCAGAATTCTAAGATTGCCTCATCTCAGCAGGTCACAGCTG | | |
| | AGATACCAGTTGATCTGAATACCAGGGAAGTCATCAGAGAATCAGGTAAATGCCCTGG | | |
| | AAATATTACAGAAGAAAGCCATGATTCAGTTTATTCTTCTGTTACTCAGAACAGACAT | | |
| | TTTCTCCCCTCTACCAGCACAAAAGTATGTGAATTTGAAAACCAAGTTGTAATTTTAA | | |
| | ATAAAAAACACAGTTTTCCAGCACTTGAGGGAGGAGAGGTCACTGCTCAGTCCTGTTG | | |
| | CGGTGCTTCCTCAGACAGCACTGAGTCTGGGAAGTCTCTCCTCTTTCGTGAATCTGAG | | |
| | GCACGAGAGGAAGAAGAGCTGGATCAGAATACGGTTCTGAGGCAGACCATCAATGTAA | | |
| | GCCTTGAGAAAGACATGCCAGGGGAAAGTGCTGTTTCTTTGAAATCCAGATCAGTAGA | | |
| | TCGTAGAGTAAGCAGCCCAGTGATGGTGGCCCAGGGTGGTGGCCCAACCCCTAAGTGG | | |
| | GAAGGGAAAAATGAAACTGGGCTTCTTGAAAAAGGTCTTCGTCCCAAAGATAGCTCAG | | |
| | AAGAGTTTAAGCTTCCAGGTACAAAGCCTGCATATGAAAGGTTCCAGTTAGTTGCATG | | |
| | CCCTCAGGAAAGAAACCCCAGTGAATGCAAGTCACAAGAAATGTTAAATCCCAACAGA | | |
| | GAACCTTCTGGAAAGAAACAGAATAAAAGAGTTAATAATACTGATGAAATGGCTAGGC | | |
| | TAATTAGGAGTGTAATGCAGCTGGAAAATGGCATCTTAGAAATTGAATCTAAGCAGAA | | |
| | TAAGCAGGTTCATGCTTCCCACACACCAGGAACCGATAAGGAGTTGGTGTTCCAGGAC | | |
| | CAGAAGGAGCAGGAGAAGACTGACCATGCCTTTAGGCCAGACAGCTCTGGAAACCCTT | | |
| | TGCCCTCTAAGGATCAGCCATCTTCTCCAAGACAGACAGATGATACTGTCTTTAGGGA | | |
| | TAGTGAAGCTCGAGCGATGGAGGTTAACAGCATTGGGAACCATCCCCAGOTCCAGAAA | | |
| | ATCACCCCAAACCCCTTCAGGTCAAGGGAAGGTGTACGAGAGAGTGAACCTGTGAGAG | | |
| | AGCACACCCACCCAGCTGGATCGGACAGACCTGCCAGGGATATTTGTGATTCTTTAGG | | |
| | GAAACACACAACTTGCAGAGAGTTCACCAACACTTCTCTTCACCCACAGAGAATGAAA | | |
| | GCATTGGCTAGAGCTCTGCCATTGCAACCCAGGCTAGAGAGGTCTTCTAAGAATAATG | | |
| | GCCAGTTTGTAAAAGCATCAGCAAGTCTCAAAGGGCAGCCTTGGGGCTTAGGAAGTCT | | |
| | TGAGGAATTGGAGACTGTGAAAGGTTTTCAGGAAAGCCAAGTAGCTGAACACGTAAGT | | |
| | AGTTCCAACCAAGAAGAGCCAAAAGCTCAAGGTAAAGTTGAAGAAATGCCTATGCAAA | | |
| | GGGGAGGCAGCCTTCAGGAAGAAAATAAAGTGACTCAGAAATTTCCTAGTCTCAGCCA | | |
| | GCTTTGTAGGGACACGTTTTTCAGGCAGGAAACTGTCAGCCCATTACTAAGCCGGACA | | |
| | GAATTCTGTACAGCTCCTCTTCACCAAGACCTGAGTAATACCTTGCCCTTGAATTCTC | | |
| | CAAGGTGGCCAAGAAGGTGTCTTCATGTACCTGTTGCTCTAGGCATCTCTTCACTTGA | | |
| | CTGTGTGCTGGATCTCACAATGTTGAAAATTCATAACAGTCCCTTGGTAACTGGAGTA | | |
| | GAGCATCAGGACCAGAGTACGGAGACCAGAAGCCACAGCCCCGAAGGAAATGTTAGAG | | |
| | GGCGTTCCTCTGAGGCACACACTGCCTGGTGTGGGTCTGTGCGATCCATGGCCATGGG | | |
| | ATCTCATAGTCAATCTGGTGTACCAGAGAGCATTCCTCTGGGGACAGAGGACAGGATC | | |
| | TCAGCAAGCACCAGCCCCCAAGACCATGGAAAGGACCTCAGAATCACCTTGCTGGGTT | | |
| | TCAGTACCAGTGAAGATTTTGCTTCTGAAGCCGAGGTGGCTGTACAAAAAGAAATAAG | | |
| | AGTCAGTTCACTGAACAAGGTCTCTAGCCAGCCTGAAAAGAGGGTCAGCTTCTCCTTG | | |
| | GAAGACGATAGTGACCAAGCCAGCAAGCCAAGGCAGAAGGCAGAGAAGGAGACTGAGG | | |
| | ACGTCGGACTGACCAGCGGTGTTTCCTTAGCACCTGTTTCCCTGCCGAGGGTGCCCAG | | |
| | TCCAGAGCCTAGGCTGTTGGAGCCCTCTGACCATGCATCCATGTGCCTGGCCATCTTG | | |
| | GAGGAGATCAGACAGGCAAAGGCCCAGAGAAAGCAGCTTCATGACTTTGTGGCCAGGG | | |
| | GCACAGTCCTTTCTTACTGTGAAACTTTACTAGAACCCGAATGTTCTTCAAGGGTTGC | | |
| | TGGCACGCCTCAGTGTAAACAAATAGACCAGTCATCATCAGACCAGACCAGGAATGAG | | |
| | GGTGAAGCACCGGGATTTCATGTGGCATCTCTATCTGCTGAAGCAGGGCAGATAGATC | | |
| | TGTTACCTGATGAGAGGAAAGTCCAGGCCACATCTCTGTCTGCAGACAGCTTTGAATC | | |
| | TCTGCCCAATACGGAAACTGACAGAGAGCCATGGGATCCTGTGCAGGCTTTCTCCCAT | | |
| | GCTGCTCCTGCTCAAGACAGGAAACGTCGTACTGGAGAACTGAGGCAGTTCGCGGGAG | | |
| | CAAGTGAACCATTTATATGTCACTCTAGTTCTTCTGAAATCATAGAGAAAAAGAAAGA | | |
| | TGCAACCAGAACACCTTCCTCAGCTGATCCTTTGGCCCCAGACAGTCCTCGTTCTTCA | | |
| | GCACCTGTGGAGGAGGTCAGGAGGGTAGTATCAAAGAAGGTAGTGGCTGCCTTACCTT | | |
| | CTCAGGCCCCTTATGATGATCCTAGAGTGACTCTGCATGAGCTAAGTCAGTCAGTTCC | | |
| | GCAGGAGACTGCAGAGGGCATACCCCCTGGCAGTCAGGACAGCAGCCCAGAGCATCAG | | |
| | GAACCCAGAACTCTAGACACCACATATGGAGAAGTTTCAGATAATTTGTTAGTGACTG | | |
| | CACAGGGAGAAAAAACAGCCCATTTTGAAAGTCAGTCTGTGACCTGTGATGTTCAGAA | | |
| | TTCTACAAGTGCCTCAGGGCCTAAGCAAGACCATGTCCAATGCCCTGAGGCTTCTACT | | |
| | GGCTTTGAAGAAGGTAGGGCAAGTCCCAAACAAGATACCATTCTGCCTGGAGCTCTGA | | |
| | CAAGGGTTGCACTGGAAGCTCCCACACAGCAGTGTGTGCAGTGTAAGGAGAGTGTTGG | | |
| | GTCTGGGTTGACAGAAGTCTGCAGGGCTGGCAGCAAACATTCCAGGCCAATTCCACTG | | |
| | CCAGATCAAAGACCAAGCGCAAATCCTGGGGGAATTGGGGAGGAAGCCCCATGTAGAC | | |
| | ACCCAAGGGAAGCTTTAGATGGCCCTGTCTTCTCAAGGAACCCTGAAGGCAGCAGGAC | | |
| | TCTCAGCCCGTCTAGAGGGAAAGAGAGCAGAACTCTTCCTTGCCGACAGCCATGCAGT | | |
| | TCTCAACCTGTTGCTACTCATGCTTATTCCTCCCATTCCTCTACTTTACTGTGTTTTA | | |
| | GAGATGGTGACCTAGGGAAGGAGCCTTTCAAGCCTGCCCCACATACTATCCACCCACC | | |
| | CTGTGTAGTACCTTCCAGGGCCTATGAAATGGATGAGACAGGAGAGATCTCTAGGGGA | | |
| | CCTGATGTGCACTTGACACATGGCCTTGAGCCCAAAGATGTTAACAGGGAATTTAGGC | | |
| | TAACAGAGAGCAGCACTTGTGAGCCTTCTACTGTGGCTGCTGTCCTATCTCGAGCTCA | | |
| | AGGCTGCAGATCCCCTTCTGCTCCTGACGTGAGGACAGGTTCCTTCAGCCACTCAGCT | | |
| | ACTGATGGAAGCGTGGGGTTAATAGGGGTTCCTGAGAAAAAGGTTGCTGAGAAGCAAG | | |
| | CAAGCACAGAACTTGAGGCTGCCTCTTTCCCTGCAGGCATGTACTCTGAGCCCCTGAG | | |
| | GCAGTTTAGGGACAGCTCTGTAGGTGACCAGAATGCACAGGTGTGTCAAACCAATCCA | | |
| | GAACCACCTGCAACAACTCAGGGACCACACACCCTGGATTTAAGTGAAGGGTCTGCTG | | |
| | AGAGCAAGTTGGTGGTAGAGCCACAGCATGAATGTTTAGAAAATACCACTAGATGTTT | | |
| | TTTGGAAAAGCCACAATTTTCCACTGAGTTGAGGGATCACAATCGCTTGGATTCCCAA | | |
| | GCCAAGTTTGTAGCAAGGTTAAAACATACCTGCAGCCCCCAGGAAGACAGTCCCTGGC | | |
| | AGGAAGAAGAGCAGCACAGAGACCAGGCTTCACGTGGTGGAGAAGGCTTCGCCCAGGG | | |
| | TGTGAATCCCCTTCCTGATGAAGATGGCTTAGATGGCTGTCAGATTTTAGATGCTGGG | | |
| | AGAGAGGAGGTGGCTGTGGCCAAGCCTCCTGTGTCCAAGATTTTATCACAGGGCTTCA | | |
| | AAGACCCAGCCACTGTGTCCTTGAGGCAAAATGAAACACCGCAGCCTGCTGCTCAGAG | | |
| | GAGTGGCCACCTCTACACTGGCAGAGAGCAGCCAGCACCCAACCACAGGGGCTCACTT | | |
| | CCTGTGACTACAATCTTCTCTGGCCCCAAACACTCCAGGTCCTCCCCCACACCACAGT | | |
| | TCTCAGTTGTCGGCTCTTCTCGTTCTCTTCAGGAGCTGAACTTGAGTGTGGAGCCTCC | | |
| | TTCCCCTACAGACGAAGATACACAGGGGCCTAACAGATTGTGGAACCCACATCTCAGG | | |
| | CGCTATTCCTCAGGAAAGTCAGTGGCAAGAACATCTCTCCAGGCTGAGGACAGCGATC | | |
| | AGAAAGCCTCATCTCGCTTGGATGATGGGACTACCGATCACAGGCACCTGAAGCCTGC | | |
| | CACCCCTCCTTATCCAATGCCTTCCACTCTCTCACACATGCCAACCCCTGATTTCACG | | |
| | ACCAGCTGGATGTCTGGTACTTTGGAACAAGCCCAACAGGGAAAGCGAGAGAAACTGG | | |
| | GTGTCCAGGTTAGGCCAGAAAATTGGTGCTCTCAGATGGACAAAGGAATGCTGCACTT | | |
| | TGGCTCCAGTGACATCAGTCCCTATGCGCTGCCGTGGCGTCCCGAGGAGCCTGCACGT | | |
| | ATCAGCTGGAAGCAGTATATGTCTGGCAGTGCAGTCGATGTTTCCTGCAGCCAGAAGC | | |
| | CCCAGGGGCTGACACTATCAAATGTGGCCCGGTGCTCCAGCATGGACAATGGCCTAGA | | |
| | AGACCAGAACTCCCCTTTCCACTCCCACCTCAGCACTTACGCCAATATTTGTGATCTG | | |
| | TCAACCACACACAGCAGCACTGAGAATGCCCAGGGTTCAAATGAGGCCTGGGAAGTAT | | |
| | TCCGAGGGAGTTCTTCAATTGCCTTAGGAGACCCCCACATCCCGACGAGCCCTGAAGG | | |
| | AGTAGCCCCCACTTCGGGTCATGACAGAAGGCCTCAGTTCAGGGGCCCTTCTGGTGAA | | |
| | GCAGACTGTCTGAGGAGTAAGCCCCCCTTGGCCAAAGGAAGTGCTGCAGGTCCAGTGG | | |
| | ATGAGATTATGCTGCTGTATCCATCAGAGGCAGGCTGCCCTGTGGGACAGACCAGGAC | | |
| | GAACACATTCGAACAGGGCACACAGACCCTCGGCAGCAGGCGCCACTGGAGCAGCACT | | |
| | GACATCTCCTTTGCTCAGCCTGAAGCCAGTGCAGTATCAGCCTTTGATCTGGCCTCAT | | |
| | GGACCAGCATGCACAATCTGTCTCTCCACCTCTCACAGCTCCTGCACAGTACCTCAGA | | |
| | GCTGCTTGGGAGTCTCTCCCAGCCAGATGTGGCCAGAAGGGAGCAGAACACCAAGAGG | | |
| | GACATCCCAGATAAAGCCCCACAGGCCCTGATGATGGATGGCTCTACTCAGACCACTG | | |
| | TGGATGAGGGCAGCCAGACTGACCTCACCTTACCCACCCTGTGCCTCCAGACTTCAGA | | |
| | GGCTGAACCTCAGGGAGCCAATGTGATCCTTGAAGGGCTAGGCTCAGATACCTCGACT | | |
| | GTGTCTCAAGAAGAGGGAGATGTGCCAGGGGTACCTCAGAAGAGAGAGGCAGAGGAAA | | |
| | CAGCACAGAAAATGGCTCAGCTCCTCTATCTTCAGGAAGAAAGCACTCCCTACAAGCC | | |
| | CCAGAGCCCTTCAATACCCTCATCCCACTTGAGGTTTCAGAAAGCCCCCGTTGGGCAG | | |
| | CATCTTCCTTCTGTGAGCCCCTCAGTTTCTGATGCTTTCCTGCCTCCCACCTCCCAGC | | |
| | CAGAGGAGTCATATTGCTTAGTTGTCAGCAGTCCCAGTCCCAGCTCCCCTCATTCCCC | | |
| | AGGGCTCTTTCCCACTACTTCCGAGTATCCTGGGGACTCCAGGGTCCAGAAGAAGCTG | | |
| | GGCCCCACAAGTGCTTTGTTCGTGGACAGGGCCTCCTCCCCAATCCTCACTCTTAGTG | | |
| | CCAGCACCCAAGAGCCGGGTCTTTCCCCAGGCTCTTTGACCCTCTCAGCCCCTTCAAC | | |
| | TCACCCTGTTGAAGGCCACCAGAAGCTTGACTCCAGCCCAGACCCTGTTGATGCCCCA | | |
| | AGGACTCCAATGGATAATTATTCCCAAACCACTGACGAGTTAGGTGGCTCCCAGAGAG | | |
| | GTAGAAGTTCCTTACAAAGGAGTAATGGGAGATCCTTCCTTGAGTTGCACTCCCCACA | | |
| | CAGCCCACAGCAGAGTCCAAAACTCCAATTTAGTTTCTTAGGGCAGCACCCTCAGCAG | | |
| | CTTCAGCCCAGGACAACTATCGGGGTCCAAAGCAGACTGCTGCCACCACCACTGAGGC | | |
| | ACAGGAGCCAAAGGCTGGGCAACAGCTTTGTGCCTGAGAAGGTGGCTTCCCCGGAGCA | | |
| | TTGCCCACTGAGCGGTAGGGAGCCAAGTCAGTGGCAGAGCAGGACAGAAAATGGAGGT | | |
| | GAGAGTTCAGCATCTCCAGGGGAACCACAACGCACTCTGGACCGACCTTCTTCATGGG | | |
| | GAGGCCTCCAGCACCTCAGCCCCTGCCCTGTCTCTGAGTTGACTGATACTGCAGGGCT | | |
| | CCGAGGTTCTGCCTTGGGCCTCCCTCAGGCCTGCCAACCTGAGGAGTTACTGTGCTTC | | |
| | AGTTGCCAGATGTGCATGGCCCCTGAGCACCAGCACCACAGTCTGAGGGACCTCCCGG | | |
| | TGCATAACAAATTTAGTAACTGGTGTGGGGTTCAGAAGGGCTCACCTGGGGGGTTGGA | | |
| | CATGACTGAGGAGGAGCTGGGGGCCAGCGGTGATCTCAGCTCTGAAAAGCAGGAACAG | | |
| | AGTCCCCCACAACCTCCTAATGACCACAGCCAGGATTCTGAGTGGTCCAAGAGGGAGC | | |
| | AGATCCCCCTGCAAGTTGGGGCCCAGAACCTCTCACTCAGCGTGGAACTCACAGAAGC | | |
| | GAAACTGCACCATGGCTTTGGGGAGGCCGATGCCCTGCTCCAGGTGCTGCAGAGTGGG | | |
| | ACAGGGGAGGCGCTTGCTGCTGATGAACCTGTGACATCCACCTGGAAGGAGCTCTATG | | |
| | CACGGCAAAAAAAGGCCATTGAGACCCTCAGGAGAGAGCGGGCTGAGCGACTTGGGAA | | |
| | CTTCTGCCGGACGCGAAGCCTTAGCCCTCAGAAACAACTGAGCCTCCTGCCCAACAAA | | |
| | GATCTCTTCATCTGGGATCTTGACTTGCCCAGCAGACGCCGAGAATACCTGCAGCAAC | | |
| | TGAGGAAGGATGTTGTGGAGACCACCAGGAGCCCAGAGTCAGTGTCAAGGTCAGCTCA | | |
| | CACACCCTCTGACATAGAGTTGATGCTGCAAGACTACCAGCAGGCCCATGAGGAGGCC | | |
| | AAGGTGGAGATTGCCCGGGCCCGAGACCAACTGCGGGAGCGGACTGAACAAGAGAAGC | | |
| | TGAGAATCCACCAGAAGATCATTTCCCAGCTATTGAAGGAAGAGGATAAACTACATAC | | |
| | CTTGGCCAATTCCAGCTCCCTGTGCACCAGCTCTAATGGAAGCCTCTCGTCTGGCATG | | |
| | ACCTCTGGCTATAATAGCAGCCCAGCCTTGTCAGGCCAGCTCCAGTTCCCAGAGAATA | | |
| | TGGGGCATACAAACTTGCCTGATTCCAGGGATGTATGGATAGGGGATGAGCGAGGAGG | | |
| | CCATTCTGCAGTGAGGAAGAACTCTGCCTACAGCCACAGAGCCTCCCTGGGCAGTTGC | | |
| | TGCTGTTCACCATCCAGTCTGTCCAGCTTGGGGACCTGCTTTTCCTCCTCCTACCAGG | | |
| | ATTTGGCCAAGCATGTCGTGGACACTTCTATGGCTGATGTAATGGCTGCTTGTTCGGA | | |
| | TAATTTGCACAACCTCTTCAGCTGCCAGGCAACTGCTGGCTGGAACTATCAGGGTGAG | | |
| | GAGCAGGCGGTGCAGCTTTACTACAAGGTGTTTTCTCCCACTCGGCATGGCTTCCTGG | | |
| | GGGCAGGTGTGGTGTCCCAGCCGCTGTCTCGTGTGTGGGCGGCTGTCAGTGACCCCAC | | |
| | TGTGTGGCCCCTGTATTACAAGCCCATCCAGACAGCAAGGCTGCATCAGCGAGTGACC | | |
| | AACAGCATCAGCCTGGTGTACTTGGTGTGCAACACCACCCTGTGCGCACTGAAGCAGC | | |
| | CACGGGATTTCTGTTGTGTCTGCGTGGAAGCCAAAGAGGGTCACCTGTCTGTCATGGC | | |
| | AGCCCAGTCTCTGTATGATACATCCATGCCAAGACCCAGCAGAAAAATGGTTCACGGG | | |
| | GAGATCCTGCCCAGTGCCTGGATCTTGCAGCCCATCACTGTGGAAGGGAAGGAAGTCA | | |
| | CCAGAGTCATCTACTTGGCCCAGGTGGAACTTGGTGCTCCAGGCTTCCCACCTCAGCT | | |
| | CCTGAGCTCTTTCATCAAACGGCACCCACTGGTTATAGCCAGACTGGCTTCCTTCCTT | | |
| | GTGCAGGAAAAGCTGATGCTACCTGCTGTGGCCGATTGGGGCAGACAGCACTGGCCCA | | |
| | GGGATGCTAGCAAAGCCCAGTCAGTACTTGGTCACAGCTGGCACCAGTGCAGAGCAAA | | |
| | CGGCCTGAGCTCCTGGCCCAGACTATCCAGAGTGAATGCAGCTCTGCTCACCTTTTGG | | |
| | ATTTCTCACCTTTCTTTCCTGTTTCTGGGACTCTGCGGCAGACAGGACACTTAAGGAC | | |
| | CAGGACTGGCCACAGCCAGCAGAGCCGGGGACTGCAGTGCTTTGGCAAGGTGCTTCCG | | |
| | CAGGCTGGTAGGGAA | | |
| | ORF Start: ATG at 1 | | ORF Stop: TAA at 14320 |
| | SEQ ID NO:322 | 4773 aa | MW at 524614.9 kD |

| | |
|---|---|
| NOV34a, | MANVQVAVRVRPLSKRETKEGGRIIVEVDGKVAKIRNLKVDNRPDGFGDSREKVMAFG |
| CG157505-01 | |
| Protein Sequence | FDYCYWSVNPEDPQYASQDVVFQDLGMEVLSGVAKGYNICLFAYGQTGSGKTYTMLGT |
| | PASVGLTPRICEGLFVREKDCASLPSSCRIKVSFLETYNERVRDLLKQSGQKKSYTLR |
| | VREHPEMGPYVQGLSQHVVTNYKQVIQLLEEGIANRITAATHVHEASSRSHAIFTIHY |
| | TQAILENNLPSEMASKINLVDLAGSERADPSYCKDRIAEGANINKSLVTLGTVISTLA |
| | QNSQVFSSCQSLNSSVSNGGDSGILSSPSGTSSGGAPSRRQSYIPYRDSVLTWLLKDS |
| | LGGNSKTIMVASVSPAHTSYSETMSTLRYASSAKNIINKPRVNEDANLKLIRELREEI |
| | ERLKALLLSFELRNFSSLSDENLKELVLQNELKTDQLTKDWTQKWNDWQALMEHYSVD |
| | INRRRAGVVIDSSLPHLMALEDDVLSTGVVLYHLKEGTTKIGRIDSDQEQDIVLQGQW |
| | IERDHCTTTSACGVVVLRPARGARCTVNGREVTASCRLTQGAVITLGKAQKFRFNHPA |
| | EAAVLRQRRQVGEAAAGRGSLEWLDLDGDLAASRLGLSPLLWKERRALEEQCDEDHQT |
| | PRDGETSHRAQIQQQQSYVEDLRHQILAEEIRAAKELEFDQAWISQQIKENQQCLLRE |
| | ETWLASLQQQQQEDQVAEKELEASVALDAWLQTDPEIQPSPFVQSQKRVVHLQLLRRH |
| | TLRAAERNVRRKKVSFQLERIIKKQRLLEAQKRLEKLTTLCWLQDDSTQEPPYQVLSP |
| | DATVPRPPCRSKLTSCSSLSPQRLCSKHMPQLHSIFLSWDPSTTLPPRPDPTHQTSEK |
| | TSSEEHLPQAASYPARTGCLRKNGLHSSGHGQPCTARAALARKGASAPDACLTMSPNS |
| | VGIQEMEMGVKQPHQMVSQGLASLRKSANKLKPRHEPKIFTSTTQTRGAKGLADPSHT |
| | QAGWRKEGNLGTHKAAKCASCNSLYPHGPRQTAGHGKAVKTFWTEYKPPSPSRASKRH |
| | QRVLATRVRNITKKSSHLPLGSPLKRQQNTRDPDTMVPLTDFSPVMDHSREKDNDLSD |
| | TDSNYSLDSLSCVYAKALIEPLKPEERKWDFPEPENSESDDSQLSEDSLAEKRYQSPK |
| | NRLGGNRPTNNRGQPRTRTRASVRGFTAASDSDLLAQTHRSFSLDSLIDAEEELGEDQ |
| | QEEPFPGSADEIPTETFWHLEDSSLPVMDQEAICRLGPINYRTAARLDAVLPMSSSFY |
| | LDPQFQPHCELQPHCELQPHCELQPHCEQAESQVEPSYSEQADSLQGMQLSRESPLMS |
| | MDSWFSCDSKINPSSPPGIVGSLCPSPDMQEFHSCKGERPGYWPNTEELKPSDAETVL |
| | PYSSKLHQGSTELLCSARDEHTASAADTSRLSLWGIQRLIQPGADGTFQGRCIPDMTQ |
| | QGSSEASHNSSVSNVLAASATTLTHVGSTHERDWSALQQKYLLELSCPVLEAIGAPKP |
| | AYPYLEEDSGSLAQASSKGGDTLLPVGPRVSSNLNLNNFPVHLSRIRRLRAEKEQDSL |
| | NAKLEGVSDFFSTSEKEASYDETYSADLESLSASRSTNAQVFATENAIPDSMTEACEV |
| | KQNNLEECLQSCRKPGLMTSSDEDFFQKNACHSNVTTATKADHWSQGWAPLRKNSAVQ |
| | PGQLSPDSHYPLEEEKTDCQESSKEAVRRHINVSFALPSGPELYLHSAPWNPLSSSLQ |
| | PPLLETFYVTKSRDALTETALEIPACREVRVPSPPPREAWGFGHNHQALQGAYLKNNL |
| | PVLLQNQNSKIASSQQVTAEIPVDLNTREVIRESGKCPGNITEESHDSVYSSVTQNRH |
| | FLPSTSTKVCEFENQVVILNKKHSFPALEGGEVTAQSCCGASSDSTESGKSLLFRESE |
| | AREEEELDQNTVLRQTINVSLEKDMPGESAVSLKSRSVDRRVSSPVMVAQCGGPTPKW |
| | EGKNETGLLEKGLRPKDSSEEFKLPGTKPAYERFQLVACPQERNPSECKSQEMLNPNR |
| | EPSGKKQNKRVNNTDEMARLIRSVMQLENGILEIESKQNKQVHASHTPGTDKELVFQD |
| | QKEQEKTDHAFRPDSSGNPLPSKDQPSSPRQTDDTVFRDSEAGAMEVNSIGNHPQVQK |
| | ITPNTFRSREGVRESEPVREHTHPAGSDRPARDICDSLGKHTTCREFTNTSLHPQRMK |
| | ALARALPLQPRLERSSKNNCQFVKASASLKGQPWGLGSLEELETVKGFQESQVAEHVS |
| | SSNQEEPKAQGKVEEMPMQRGGSLQEENKVTQKFPSLSQLCRDTFFRQETVSPLLSRT |
| | EFCTAPLHQDLSNTLPLNSPRWPRRCLHVPVALGISSLDCVLDLTMLKIHNSPLVTGV |
| | EHQDQSTETRSHSPECNVRGRSSEAHTAWCGSVRSMANGSHSQSGVPESIPLGTEDRI |
| | SASTSPQDHGKDLRITLLCFSTSEDFASEAEVAVQKEIRVSSLNKVSSQPEKRVSFSL |
| | EEDSDQASKPRQKAEKETEDVGLTSGVSLAPVSLPRVPSPEPRLLEPSDHASMCLAIL |
| | EEIRQAKAQRKQLHDFVARGTVLSYCETLLEPECSSRVAGRPQCKQIDQSSSDQTRNE |
| | GEAPGFHVASLSAEAGQIDLLPDERKVQATSLSADSFESLPNTETDREPWDPVQAFSH |
| | AAPAQDRKRRTGELRQFAGASEPFICHSSSSEIIEKKKDATRTPSSADPLAPDSPRSS |
| | APVEEVRRVVSKKVVAALPSQAPYDDPRVTLHELSQSVPQETAEGIPPGSQDSSPEHQ |
| | EPRTLDTTYGEVSDNLLVTAQGEKTAHFESQSVTCDVQNSTSASGPKQDHVQCPEAST |
| | GFEEGRASPKQDTILPGALTRVALEAPTQQCVQCKESVGSGLTEVCRAGSKHSRPIPL |
| | PDQRPSANPGGICEEAPCRHPREALDGPVFSRNPEGSRTLSPSRGKESRTLPCRQPCS |
| | SQPVATHAYSSHSSTLLCFRDGDLGKEPFKAAPHTIHPPCVVPSRAYEMDETGEISRG |
| | PDVHLTHGLEPKDVNREFRLTESSTCEPSTVAAVLSRAQCCRSPSAPDVRTGSFSHSA |
| | TDGSVGLIGVPEKKVAEKQASTELEAASFPAGMYSEPLRQFRDSSVGDQNAQVCQTNP |
| | EPPATTQGPHTLDLSEGSAESKLVVEPQHECLENTTRCFLEKPQFSTELRDHNRLDSQ |
| | AKFVARLKHTCSPQEDSPWQEEEQHRDQASGGGEGFAQGVNPLPDEDGLDGCQILDAG |
| | REEVAVAKPPVSKILSQCFKDPATVSLRQNETPQPAAQRSGHLYTGREQPAPNHRGSL |
| | PVTTIFSGPKHSRSSPTPQFSVVGSSRSLQELNLSVEPPSPTDEDTQGPNRLWNPHLR |
| | GYSSGKSVARTSLQAEDSDQKASSRLDDGTTDHRHLKPATPPYPMPSTLSHMPTPDFT |
| | TSWMSGTLEQAQQGKREKLGVQVRPENWCSQMDKGMLHFGSSDISPYALPWRPEEPAR |
| | ISWKQYMSGSAVDVSCSQKPQGLTLSNVARCSSMDNGLEDQNSPFHSHLSTYANICDL |
| | STTHSSTENAQGSNEAWEVFRGSSSIALGDPHIPTSPEGVAPTSGHDRRPQFRGPSGE |
| | ADCLRSKPPLAKCSAAGPVDEIMLLYPSEAGCPVGQTRTNTFEQGTQTLGSRRHWSST |
| | DISFAQPEASAVSAFDLASWTSMHNLSLHLSQLLHSTSELLGSLSQPDVARREQNTKR |
| | DIPDKAPQALMMDGSTQTTVDEGSQTDLTLPTLCLQTSEAEPQGANVILEGLGSDTST |
| | VSQEEGDVPGVPQKREAEETAQKMAQLLYLQEESTPYKPQSPSIPSSHLRFQKAPVGQ |
| | HLPSVSPSVSDAFLPPSSQPEESYCLVVSSPSPSSPHSPGLFPSTSEYPGDSRVQKKL |
| | GPTSALFVDRASSPILTLSASTQEPGLSPGSLTLSAPSTHPVEGHQKLDSSPDPVDAP |
| | RTPMDNYSQTTDELGGSQRGRSSLQRSNGRSFLELHSPHSPQQSPKLQFSFLGQHPQQ |
| | LQPRTTIGVQSRLLPPPLRHRSQRLGNSFVPEKVASPEHCPLSGREPSQWQSRTENGG |
| | ESSASPGEPQRTLDRPSSWGGLQHLSPCPVSELTDTAGLRGSALGLPQACQPEELLCF |
| | SCQMCMAPEHQHHSLRDLPVHNKFSNWCGVQKGSPGGLDMTEEELGASGDLSSEKQEQ |
| | SPPQPPNDHSQDSEWSKREQIPLQVGAQNLSLSVELTEAKLHHGFGEADALLQVLQSG |
| | TGEALAADEPVTSTWKELYARQKKAIETLRRERAERLGNFCRTRSLSPQKQLSLLPNK |
| | DLFIWDLDLPSRRREYLQQLRKDVVETTRSPESVSRSAHTPSDIELMLQDYQQAHEEA |
| | KVEIARARDQLRERTEQEKLRIHQKIHSQLLKEEDKLHTLANSSSLCTSSNGSLSSGM |
| | TSGYNSSPALSGQLQFPENMGHTNLFDSRDVWIGDERGGHSAVRKNSAYSHRASLGSC |
| | CCSPSSLSSLGTCFSSSYQDLAKHVVDTSMADVMAACSDNLHNLFSCQATAGWNYQGE |
| | EQAVQLYYKVFSPTRHGFLGAGVVSQPLSRVWAAVSDPTVWPLYYKPIQTARLHQRVT |
| | NSISLVYLVCNTTLCALKQPRDFCCVCVEAKEGHLSVMAAQSVYDTSMPRPSRKMVHG |
| | EILPSAWILQPITVEGKEVTRVIYLAQVELGAPGFPPQLLSSFIKRQPLVIARLASFL |
| | VQEKLMLPAVADWGRQHWPRDASKAQSVLGNSWHQCRANGLSSWPRLSRVNAALLTFW |
| | ISHLSFLFLGLCGRQDT |

Further analysis of the NOV34a protein yielded the following properties shown in Table 34B.

TABLE 34B
Protein Sequence Properties NOV34a

| | |
|---|---|
| PSort | 0.9000 probability located in nucleus; 0.6640 probability |
| analysis: | located in plasma membrane; 0.3694 probability located |
| | in mitochondrial inner membrane; 0.3000 probability |
| | located in microbody (peroxisome) |
| SignalP | No Known Signal Sequence Predicted |
| analysis: | |

A search of the NOV34a protein against the Geneseq database, a proprietary database that contains sequences published in patents and patent publication, yielded several homologous proteins shown in Table 34C.

TABLE 34C
Geneseq Results for NOV34a

| | | NOV34a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAU74557 | Human kinesin motor protein | 1 . . . 590 | 518/591 (87%) | 0.0 |
| | HsKif16a - Homo sapiens, 563 aa. | 1 . . . 563 | 519/591 (87%) | |
| | [US6333184-B1, 25 DEC. 2001] | | | |
| AAU74558 | Human kinesin motor protein | 1 . . . 385 | 334/385 (86%) | 0.0 |
| | HsKif16a motor domain - Homo | 1 . . . 357 | 335/385 (86%) | |
| | sapiens, 357 aa. [US6333184-B1, | | | |
| | 25 DEC. 2001] | | | |
| ABB61704 | Drosophila melanogaster | 23 . . . 784 | 306/782 (39%) | e−132 |
| | polypeptide SEQ ID NO 11904 - | 4 . . . 707 | 439/782 (56%) | |
| | Drosophila melanogaster, 1174 aa. | | | |
| | [WO200171042-A2, 27 SEP. 2001] | | | |
| AAM40034 | Human polypeptide SEQ ID NO | 2 . . . 737 | 295/804 (36%) | e−117 |
| | 3179 - Homo sapiens, 893 aa. | 4 . . . 763 | 416/804 (51%) | |
| | [WO200153312-A1, 26 JUL. 2001] | | | |
| ABP51294 | Human MDDT SEQ ID NO 316 - | 2 . . . 609 | 248/619 (40%) | e−114 |
| | Homo sapiens, 757 aa. | 19 . . . 591 | 355/619 (57%) | |
| | [WO200240715-A2, 23 MAY | | | |
| | 2002] | | | |

In a BLAST search of public sequence datbases, the NOV34a protein was found to have homology to the proteins shown in the BLASTP data in Table 34D.

TABLE 34D
Public BLASTP Results for NOV34a

| | | NOV34a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9P2P6 | KIAA1300 protein - Homo | 2881 . . . 4698 | 1818/1818 (100%) | 0.0 |
| | sapiens (Human), 1820 aa | 1 . . . 1818 | 1818/1818 (100%) | |
| | (fragment). | | | |
| Q9H6S2 | CDNA: FLJ21936 fis, clone | 1080 . . . 1883 | 802/804 (99%) | 0.0 |
| | HEP04408 - Homo sapiens | 1 . . . 804 | 802/804 (99%) | |
| | (Human), 818 aa (fragment). | | | |
| Q9DDA6 | Kinesin-like protein - Xenopus | 1 . . . 1285 | 617/1321 (46%) | 0.0 |
| | laevis (African clawed frog), | 1 . . . 1269 | 825/1321 (61%) | |
| | 1499 aa (fragment). | | | |
| Q15885 | Partial cDNA sequence, clone | 1428 . . . 1807 | 378/380 (99%) | 0.0 |
| | x529, unknown open reading | 1 . . . 380 | 378/380 (99%) | |
| | frame - Homo sapiens (Human), | | | |
| | 380 aa (fragment). | | | |
| AAH32885 | Hypothetical protein - Mus | 4340 . . . 4698 | 284/370 (76%) | e−158 |
| | musculus (Mouse), 371 aa | 1 . . . 369 | 315/370 (84%) | |
| | (fragment). | | | |

PFam analysis predicts that the NOV34a protein contains the domains shown in the Table 34E.

TABLE 34E
Domain Analysis of NOV34a

| Pfam | NOV34a | Identities/Similarities | Expect |
|---|---|---|---|
| Domain | Match Region | for the Matched Region | Value |
| kinesin | 9 . . . 295 | 122/340 (36%) | 3.1e−85 |
| | | 219/340 (64%) | |
| kinesin | 332 . . . 413 | 52/83 (63%) | 7e−41 |
| | | 72/83 (87%) | |
| FHA | 503 . . . 569 | 24/80 (30%) | 0.0059 |
| | | 46/80 (58%) | |
| REV | 4268 . . . 4335 | 16/69 (23%) | 0.52 |
| | | 43/69 (62%) | |
| START | 4496 . . . 4704 | 45/254 (18%) | 0.012 |
| | | 138/254 (54%) | |

### Example 35

The NOV35 clone was analyzed, and the nucleotide and encoded polypeptide sequences are shown in Table 35A.

TABLE 35A
NOV35 Sequence Analysis

| | SEQ ID NO:323 | 2039 bp | |
|---|---|---|---|
| NOV35a, | CTAAGAGTGGTTCCTCGCAGCTTAAAGGGAGGCACTTTTCACACTCTGTCTTAAAATC | | |
| CG157629-01 | | | |
| DNA Sequence | AGAAGTTGAATTCATGAACACATATGATTTAGATAGAAGTCATGGGATGCAGCAGTTC | | |
| | TTCAACGAAAACCAGGAGATCTGACACATCACTGAGAGCTGCGTTGATCATCCAGAAC | | |
| | TGGTACCGAGGTTACAAAGCTCGACTGAAGGCCAGACAACACTATGCCCTCACCATCT | | |
| | TCCAGTCCATCGAATATGCTGATGAACAAGGCCAAATGCAGTTATCCACCTTCTTTTC | | |
| | CTTCATGTTGGAAAACTACACACATATACATAAGGAAGAGCTAGAATTAAGAAATCAG | | |
| | TCTCTTGAAAGCGAACAGGACATGAGGGATAGATGGGATTATGTGGACTCGATAGATG | | |
| | TCCCAGACTCCTATAATGGTCCTCGGCTACAATTTCCTCTCACTTGTACGGATATTGA | | |
| | TTTACTTCTTGAGGCCTTCAAGGAACAACAGATACTTCATGCCCATTATGTCTTAGAG | | |
| | GTGCTATTTGAAACCAAGAAAGTCCTGAAGCAAATGCCGAATTTCACTCACATACAAA | | |
| | CTTCTCCCTCCAAAGAGGTAACAATCTGTGGTGATTTGCATGGGAAACTGGATGATCT | | |
| | TTTTTTGATCTTCTACAAGAATGGTCTCCCCTCAGAGAGGAACCCGTATGTTTTTAAT | | |
| | GGTGACTTTGTAGATCGAGGAAAGAATTCCATAGAGATCCTAATGATCCTGTGTGTGA | | |
| | GTTTTCTTGTCTACCCCAATGACCTGCACTTGAACAGAGGGAACCACGAAGATTTTAT | | |
| | GATGAATCTGAGGTATGGCTTCACGAAAGAAATTTTGCATAAATATAAGCTACATGGA | | |
| | AAAAGAATCTTACAAATCTTCGAAGAATTCTATGCCTGGCTCCCAACGGAAACAAACA | | |
| | GAGACCATGGCACTGACTCGAAGCACAATAAAGTAGGTGTGACTTTTAATGCACATGG | | |
| | AAGAATCAAAACAAATGGATCTCCTACTGAACACTTAACAGAGCATGAATGGGAACAG | | |
| | ATTATTGATATTCTGTGGAGTGATCCCAGAGGCAAAAATGGCTGTTTTCCAAATACGT | | |
| | GCCGAGGAGGGGGCTGCTATTTTGGACCAGATGTTACTTCCAAGATTCTTAATAAATA | | |
| | CCAGTTGAAGATGCTCATCAGGTCTCATGAATGTAAGCCCGAAGGGTATGAAATCTGT | | |
| | CATGATGGGAAGGTGGTGACTATATTTTCTGCTTCTAATTATTATGAAGAAGGCAGCA | | |
| | ATCGAGGAGCTTACATCAAACTATGTTCTGGTACAACTCCTCGATTTTTCCAGTACCA | | |
| | AGTAACTAAAGCAACGTGCTTTCAGCCTCTTCGCCAAAGAGTGGATACTATGGAAAAC | | |
| | AGCGCCATCAAGATATTAAGAGAGAGAGTGATTTCACGAAAAAGTGACCTTACTCGTG | | |
| | CTTTCCAACTTCAAGACCACAGAAAATCAGGAAAACTTTCTGTGAGCCAGTGGGCTTT | | |
| | TTGCATGGAGAACATTTTGGGGCTGAACTTACCATGGAGATCCCTCAGTTCGAATCTG | | |
| | GTAAACATAGACCAAAATGGAAACGTTGAATACATGTCCAGCTTCCAGAATATCCGCA | | |
| | TTGAAAAACCTGTACAAGAGGCTCATTCTACTCTAGTTGAAACTCTGTACAGATACAG | | |
| | ATCTGACCTGGAAATCATATTTAATGCCATTGACACTGATCACTCAGGCCTGATCTCC | | |
| | GTGGAAGAATTTCGTGCCATGTGGAAACTTTTTAGTTCTCACTACAATGTTCACATTG | | |
| | ATGATTCCCAAGTCAATAAGCTTGCCAACATAATGGACTTGAACAAAGATGGAAGCAT | | |
| | TGACTTTAATGAGTTTTTAAAGGCTTTCTATGTAGTGCATAGATATGAAGACTTGATG | | |
| | AAACCTGATGTCACCAACCTTGGCTAAACACAAATGAGAGCTTCCCTCAGGCTCCCTG | | |
| | AAACAGCTAGGCCCAAATCACAAGTACAGTCCTTTCCAACACCCCTGAAATTCATAGT | | |
| | CAGTAGCAG | | |
| | ORF Start: ATG at 100 | | ORF Stop: TAA at 1939 |
| | SEQ ID NO:324 | 613 aa | MW at 71315.2 kD |
| NOV35a, | MGCSSSSTKTRRSDTSLRAALIIQNWYRGYKARLKARQHYALTIFQSIEYADEQGQMQ | | |
| CG157629-01 | | | |
| Protein Sequence | LSTFFSFMLENYTHIHKEELELRNQSLESEQDMRDRWDYVDSIDVPDSYNGPRLQFPL | | |
| | TCTDIDLLLEAFKEQQILHAHYVLEVLFETKKVLKQMPNFTHIQTSPSKEVTICGDLH | | |
| | GKLDDLFLTFYKNGLPSERNPYVFNGDFVDRGKNSIEILMILCVSFLVYPNDLHLNRG | | |
| | NHEDFMMNLRYGFTKEILHKYKLHGKRILQILEEFYAWLPTETNRDHGTDSKHNKVGV | | |
| | TFNAHGRIKTNGSPTEHLTEHEWEQIIDILWSDPRGKNGCFPNTCRGGGCYFGPDVTS | | |
| | KILNKYQLKMLIRSHECKPECYEICHDGKVVTIFSASNYYEEGSNRCAYIKLCSGTTP | | |
| | RFFQYQVTKATCFQPLRQRVDTMENSAIKILRERVISRKSDLTRAFQLQDHRKSGKLS | | |
| | VSQWAFCMENILGLNLPWRSLSSNLVNIDQNGNVEYMSSFQNIRIEKPVQEAHSTLVE | | |
| | TLYRYRSDLEIIFNAIDTDHSGLISVEEFRAMWKLFSSHYNVHIDDSQVNKLANIMDL | | |
| | NKDGSIDFNEFLKAFYVVHRYEDLMKPDVTNLG | | |
| | SEQ ID NO:325 | 2039 bp | |
| NOV35b, | CTAAGAGTGGTTCCTCGCACCTTAAAGGGAGGCACTTTTCACACTCTGTCTTAAAATC | | |
| CG157629-01 | | | |
| DNA Sequence | AGAAGTTGAATTCATGAACACATATGATTTAGATAGAAGTCATGGGATGCAGCAGTTC | | |
| | TTCAACGAAAACCAGGAGATCTGACACATCACTGAGAGCTGCGTTGATCATCCAGAAC | | |
| | TGGTACCGAGGTTACAAAGCTCGACTGAAGGCCAGACAACACTATGCCCTCACCATCT | | |
| | TCCAGTCCATCGAATATGCTGATGAACAAGGCCAAATGCAGTTATCCACCTTCTTTTC | | |
| | CTTCATGTTGGAAAACTACACACATATACATAAGGAAGAGCTAGAATTAAGAAATCAG | | |
| | TCTCTTGAAAGCGAACAGGACATGAGGGATAGATGGGATTATGTGGACTCGATAGATG | | |
| | TCCCAGACTCCTATAATGGTCCTCGGCTACAATTTCCTCTCACTTGTACGGATATTGA | | |
| | TTTACTTCTTGAGGCCTTCAAGGAACAACAGATACTTCATGCCCATTATGTCTTAGAG | | |
| | GTGCTATTTGAAACCAAGAAAGTCCTGAAGCAAATGCCGAATTTCACTCACATACAAA | | |
| | CTTCTCCCTCCAAAGAGGTAACAATCTGTGGTGATTTGCATGGGAAACTGGATGATCT | | |
| | TTTTTTGATCTTCTACAAGAATGGTCTCCCCTCAGAGAGGAACCCGTATGTTTTTAAT | | |
| | GGTGACTTTGTAGATCGAGGAAAGAATTCCATAGAGATCCTAATGATCCTGTGTGTGA | | |
| | GTTTTCTTGTCTACCCCAATGACCTGCACTTGAACAGAGGGAACCACGAAGATTTTAT | | |
| | GATGAATCTGAGGTATGGCTTCACGAAAGAAATTTTGCATAAATATAAGCTACATGGA | | |
| | AAAAGAATCTTACAAATCTTGGAAGAATTCTATGCCTGGCTCCCAACGCAAACAAACA | | |
| | GAGACCATGGCACTGACTCGAAGCACAATAAAGTAGGTGTGACTTTTAATGCACATGG | | |
| | AAGAATCAAAACAAATGGATCTCCTACTGAACACTTAACAGAGCATGAATGGGAACAG | | |
| | ATTATTGATATTCTGTGGAGTGATCCCAGAGGCAAAAATGGCTGTTTTCCAAATACGT | | |
| | GCCGAGGAGGGGGCTGCTATTTTGGACCAGATGTTACTTCCAAGATTCTTAATAAATA | | |
| | CCAGTTGAAGATGCTCATCAGGTCTCATGAATGTAAGCCCGAAGGGTATGAAATCTGT | | |
| | CATGATGGGAAGGTGGTGACTATATTTTCTGCTTCTAATTATTATGAAGAAGGCAGCA | | |
| | ATCGAGGAGCTTACATCAAACTATCTTCTGGTACAACTCCTCGATTTTTCCAGTACCA | | |
| | AGTAACTAAAGCAACGTGCTTTCAGCCTCTTCGCCAAAGAGTGGATACTATGGAAAAC | | |
| | AGCGCCATCAAGATATTAAGAGAGAGAGTGATTTCACGAAAAAGTGACCTTACTCGTG | | |
| | CTTTCCAACTTCAAGACCACAGAAAATCAGGAAAACTTTCTGTGAGCCAGTGGGCTTT | | |
| | TTGCATGGAGAACATTTTGGGGCTGAACTTACCATGGAGATCCCTCAGTTCGAATCTG | | |
| | GTAAACATAGACCAAAATGGAAACGTTGAATACATGTCCAGCTTCCAGAATATCCGCA | | |
| | TTGAAAAACCTGTACAAGAGGCTCATTCTACTCTAGTTGAAACTCTGTACAGATACAG | | |
| | ATCTGACCTGGAAATCATATTTAATGCCATTGACACTGATCACTCAGGCCTGATCTCC | | |
| | GTGGAAGAATTTCGTGCCATGTGGAAACTTTTTAGTTCTCACTACAATGTTCACATTG | | |
| | ATGATTCCCAAGTCAATAAGCTTGCCAACATAATGGACTTGAACAAAGATGGAAGCAT | | |
| | TGACTTTAATGAGTTTTTAAAGGCTTTCTATGTAGTGCATAGATATGAAGACTTGATG | | |
| | AAACCTGATGTCACCAACCTTGGCTAAACACAAATGAGAGCTTCCCTCAGGCTCCCTG | | |
| | AAACAGCTAGGCCCAAATCACAAGTACAGTCCTTTCCAACACCCCTGAAATTCATAGT | | |
| | CAGTAGCAG | | |
| | ORF Start: ATG at 100 | | ORF Stop: TAA at 1939 |
| | SEQ ID NO:326 | 613 aa | MW at 71315.2 kD |

| | |
|---|---|
| NOV35b, | MGCSSSSTKTRRSDTSLRAALIIQNWYRGYKARLKARQHYALTIFQSIEYADEQOQMQ |
| CG157629-01 | |
| Protein Sequence | LSTFFSFMLENYTHIHKEELELRNQSLESEQDMRDRWDYVDSIDVPDSYNGPRLQFPL |
| | TCTDIDLLLEAFKEQQILHAHYVLEVLFETKKVLKQMPNFTHIQTSPSKEVTICGDLH |
| | GKLDDLFLIFYKNGLPSERNPYVFNGDFVDRGKNSIEILMILCVSFLVYPNDLHLNRG |
| | NHEDFMMNLRYGFTKEILHKYKLHGKRILQILEEFYAWLPTETNRDHGTDSKHNKVGV |
| | TFNAHGRIKTNGSPTEHLTEHEWEQIIDILWSDPRGKNGCFPNTCRGGGCYFGPDVTS |
| | KILNKYQLKMLIRSHECKPEGYEICHDGKVVTIFSASNYYEEGSNRGAYIKLCSGTTP |
| | RFFQYQVTKATCFQPLRQRVDTMENSAIKILRERVISRKSDLTNAFQLQDHRKSGKLS |
| | VSQWAFCMENILGLNLPWRSLSSNLVMIDQNGNVEYMSSFQNIRIEKPVQEAHSTLVE |
| | TLYRYRSDLEIIFNAIDTDHSGLISVEEFRAMWKLFSSHYNVHIDDSQVNKLANIMDL |
| | NKDGSIDFNEFLKAFYVVHRYEDLMKPDVTNLG |

Sequence comparison of the above protein sequences yields the following sequence relationships shown in Table 35B.

TABLE 35B
Comparison of NOV35a against NOV35b.

| | | Identities/ |
|---|---|---|
| | | Similarities for |
| Protein | NOV35a Residues/ | the Matched |
| Sequence | Match Residues | Region |
| NOV35b | 1 . . . 613 | 613/613 (100%) |
| | 1 . . . 613 | 613/613 (100%) |

Further analysis of the NOV35a protein yielded the following properties shown in Table 35C.

TABLE 35C
Protein Sequence Properties NOV35a

| | |
|---|---|
| PSort | 0.8171 probability located in mitochondrial matrix space; |
| analysis: | 0.4962 probability located in mitochondrial inner membrane; |
| | 0.4962 probability located in mitochondrial intermembrane |
| | space; 0.4962 probability located in mitochondrial outer |
| | membrane |
| SignalP | No Known Signal Sequence Predicted |
| analysis: | |

A search of the NOV35a protein against the Geneseq database, a proprietary database that contains sequences published in patents and patent publication, yielded several homologous proteins shown in Table 35D.

TABLE 35D
Geneseq Results for NOV35a

| | | | Identities/ | |
|---|---|---|---|---|
| | | | Similarities for | |
| Geneseq | Protein/Organism/Length | NOV35a Residues/ | the Matched | Expect |
| Identifier | [Patent #, Date] | Match Residues | Region | Value |
| AAB47250 | Human PP7 - Homo sapiens, 653 aa. | 1 . . . 613 | 612/653 (93%) | 0.0 |
| | [WO200130830-A2, 03 MAY | 1 . . . 653 | 612/653 (93%) | |
| | 2001] | | | |
| ABB71489 | Drosophila melanogaster | 44 . . . 602 | 231/578 (39%) | e−117 |
| | polypeptide SEQ ID NO 41259 - | 9 . . . 580 | 341/578 (58%) | |
| | Drosophila melanogaster, 637 aa. | | | |
| | [WO200171042-A2, 27 SEP. 2001] | | | |
| AAE09722 | Novel cell cycle protein, protein | 86 . . . 422 | 126/343 (36%) | 3e−57 |
| | phosphatase type 5 (PP5) - | 156 . . . 487 | 194/343 (55%) | |
| | Unidentified, 499 aa. | | | |
| | [WO200164913-A2, 07 SEP. 2001] | | | |
| AAE09733 | Protein phosphatase type 5 (PP5) | 86 . . . 422 | 125/343 (36%) | 2e−56 |
| | variant, N303A - Unidentified, 499 | 156 . . . 487 | 193/343 (55%) | |
| | aa. [WO200164913-A2, 07 SEP. | | | |
| | 2001] | | | |
| ABG09989 | Novel human diagnostic protein | 86 . . . 422 | 125/343 (36%) | 3e−56 |
| | #9980 - Homo sapiens, 500 aa. | 160 . . . 491 | 193/343 (55%) | |
| | [WO200175067-A2, 11 OCT. 2001] | | | |

In a BLAST search of public sequence datbases, the NOV35a protein was found to have homology to the proteins shown in the BLASTP data in Table 35E.

TABLE 35E
Public BLASTP Results for NOV35a

| | | | Identities/ | |
|---|---|---|---|---|
| Protein | | | Similarities for | |
| Accession | | NOV35a Residues/ | the Matched | Expect |
| Number | Protein/Organism/Length | Match Residues | Portion | Value |
| O14829 | Serine/threonine protein phosphatase | 1 . . . 613 | 612/653 (93%) | 0.0 |
| | with EF-hands-1 (EC 3.1.3.16) | 1 . . . 653 | 612/653 (93%) | |
| | (PPEF-1) (Protein phosphatase with | | | |
| | EF calcium-binding domain) (PPEF) | | | |
| | (Serine/threonine protein phosphatase | | | |
| | 7) (PP7) - Homo sapiens (Human), | | | |
| | 653 aa. | | | |
| O01921 | Hypothetical 80.3 kDa protein | 6 . . . 600 | 258/637 (40%) | e−131 |
| | (Protein phosphatase with EF-hands) - | 67 . . . 703 | 375/637 (58%) | |
| | Caenorhabditis elegans, 707 aa. | | | |
| T34072 | hypothetical protein F23H11.8 - | 15 . . . 600 | 252/629 (40%) | e−130 |
| | Caenorhabditis elegans, 722 aa. | 90 . . . 718 | 368/629 (58%) | |
| P40421 | Serine/threonine protein phosphatase | 14 . . . 602 | 241/608 (39%) | e−123 |
| | rdgC (EC 3.1.3.16) (Retinal | 3 . . . 604 | 360/608 (58%) | |
| | degeneration C protein) - Drosophila | | | |
| | melanogaster (Fruit fly), 661 aa. | | | |
| AAM22065 | C. elegans PEF-1 protein | 100 . . . 600 | 224/520 (43%) | e−121 |
| | (corresponding sequence F23H11.8b) - | 49 . . . 568 | 319/520 (61%) | |
| | Caenorhabditis elegans, 572 aa. | | | |

PFam analysis predicts that the NOV35a protein contains the domains shown in the Table 35F.

TABLE 35F
Domain Analysis of NOV35a

| | | Identities/ | |
|---|---|---|---|
| | | Similarities for | |
| Pfam | NOV35a | the Matched | Expect |
| Domain | Match Region | Region | Value |
| IQ | 17 . . . 37 | 9/21 (43%) | 0.0022 |
| | | 17/21 (81%) | |
| STphosphatase | 121 . . . 272 | 53/159 (33%) | 7.9e−46 |
| | | 115/159 (72%) | |
| STphosphatase | 315 . . . 416 | 37/104 (36%) | 1.5e−34 |
| | | 83/104 (80%) | |
| efhand | 530 . . . 558 | 12/29 (41%) | 3.4e−06 |
| | | 25/29 (86%) | |
| efhand | 570 . . . 598 | 8/29 (28%) | 0.0011 |
| | | 24/29 (83%) | |

### Example 36

The NOV36 clone was analyzed, and the nucleotide and encoded polypeptide sequences are shown in Table 36A.

TABLE 36A
NOV36 Sequence Analysis

| | SEQ ID NO:327 | 4037 bp | |
|---|---|---|---|
| NOV36a, | TTCACCAAAATGGCATCCTGGTTATATGAATGTCTTTGTGAAGCTGAACTTGCACAGT | | |
| CG157704-01 | | | |
| DNA Sequence | ATTATTCTCATTTCACTGCCCTTGGCCTTCAGAAAATAGATGAATTAGCCAAGATTAC | | |
| | AATGAAGGACTACTCCAAATTAGGAGTCCATGACATGAACGACCGCAAACGTCTCTTC | | |
| | CAACTTATCAAAATTATTAAGATTATGCAAGAAGAAGATAAAGCAGTCAGTATCCCAG | | |
| | AGCGTCATCTTCAGACAAGCAGCCTGCGCATCAAATCTCAGGAATTAAGATCTGGCCC | | |
| | TCGCAGACAGCTGAATTTTGATTCTCCTGCTGACAATAAAGACAGAAATGCCAGCAAT | | |
| | GATGGGTTTGAAATGTGCAGTTTATCAGATTTCTCTGCAAATGAACAGAAGTCCACTT | | |
| | ACCTAAAAGTGCTAGAACACATGCTACCAGATGATTCCCAGTACCATACAAAAACAGG | | |
| | AATTCTGAATGCCACAGCTGGTGATTCCTATGTGCAAACAGAAATCAGCACTTCACTC | | |
| | TTTTCACCAAATTACCTTTCTGCAATACTGGGGGATTGTGATATTCCCATTATTCAAA | | |
| | GAATCTCTCATGTTTCAGGGTATAACTATGGAATCCCTCATTCTTGTATCAGACAGAA | | |
| | CACTTCAGAGAAACAGAATCCTTGGACTGAGATGGAGAAAATCAGAGTTTGTGTTCGA | | |
| | AAACGCCCCCTGGGCATGAGGGAGGTACGTCGTGGAGAAATTAATATTATTACTGTAG | | |
| | AAGACAAAGAAACTCTACTTGTGCATGAGAAGAAAGAAGCAGTTGACCTCACTCAATA | | |
| | TATTCTGCAGCATGTTTTTTATTTTGATGAAGTCTTTGGTGAGGCGTGCACCAATCAG | | |
| | GATGTATACATGAAGACTACTCACCCACTTATTCAGCATATTTTCAATGGAGGCAATG | | |
| | CCACTTGCTTTGCTTATGGACAGACAGGTGCTGGAAAGACCTACACCATGATAGGAAC | | |
| | TCATGAGAACCCAGGATTGTATGCTCTAGCTGCCAAAGATATCTTCAGGCAACTAGAA | | |
| | GTGTCCCAGCCAAGAAAGCACCTCTTTGTGTGGATCAGCTTCTATGAAATTTACTGTG | | |
| | GACAGCTTTATGACCTCCTAAATAGAAGAAAAAGGCTCTTTGCAAGAGAAGATAGCAA | | |
| | GCACATGGTGCAGATAGTGGGACTGCAAGAGCTTCAGGTGGACAGTGTGGAGCTCCTC | | |
| | TTACAGGTGATCTTAAAGGGCAGCAAGGAGCGCAGCACTGGGGCCACTGGAGTTAATG | | |
| | CAGACTCCTCCCGCTCCCATGCCGTCATCCAAATTCAGATCAAAGATTCAGCCAAGAG | | |
| | GACATTTGGCAGGATCTCTTTTATTGACTTGGCTGGCAGTGAAAGAGCAGCAGATGCA | | |
| | AGGGACTCAGATAGACAGACAAAGATGGAAGGTGCAGAAATAAATCAGAGTCTACTGG | | |
| | CTCTGAAGGAATGTATCCGAGCACTGGATCAGGAACACACCCATACTCCCTTCAGGCA | | |
| | AAGCAAACTAACTCAGGTCCTGAAGGACTCTTTCATCGGCAATGCCAAAACCTGCATG | | |
| | ATCGCCAACATCTCACCAAGCCACGTGGCCACTGAACACACTCTCAACACCTTGCGCT | | |
| | ATGCTGACCGGGTCAAAGAACTAAAGAAAGGCATTAAGTGTTGCACTTCAGTTACCAG | | |
| | TCGAAATCGGACATCTGGAAACTCCTCTCCAAAACGAATTCAGAGCTCCCCTGGGGCT | | |
| | TTGTCAGAGGACAAATGTTCTCCCAAAAAAGTCAAGCTGGGATTTCAGCAGTCACTCA | | |
| | CAGTGGCAGCCCCTGGTTCCACGAGAGGGAAGGTCCATCCTCTGACCAGCCACCCACC | | |
| | CAACATTCCTTTTACTTCTGCACCTAAGGTCTCTGGTAAAAGGGGTGGCTCCAGAGGG | | |
| | AGTCCTTCACAAGAGTGGGTCATTCATGCTAGCCCTGTGAAAGGAACTGTGCGCTCTG | | |
| | GACATGTGGCCAAAAAAAAGCCAGAAGAGTCAGCACCATTGTGCTCTGAGAAAAATCG | | |
| | AATGGGCAACAAAACTGTCCTTGGGTGGGAAAGCAGCGCCTCAGGCCCAGGAGAAGGC | | |
| | CTAGTGCGTGGTAAGCTGTCCACCAAGTGCAAGAAAGTGCAGACAGTGCAGCCAGTAC | | |
| | AGAAGCAGCTTGTGTCTCGAGTTGAGCTCTCCTTTGGCAACGCCCACCACAGGGCTGA | | |
| | GTACAGTCAAGACAGCCAGAGGGGCACCCCTGCTAGGCCTGCCTCTGAAGCTTGGACA | | |
| | AACATCCCGCCACATCAGAAGGAGAGGGAGGAACATCTGCGTTTCTATCACCAGCAGT | | |
| | TCCAACAGCCACCTCTCCTCCAACAGAAGTTAAAATACCAACCACTGAAAAGGTCTTT | | |
| | ACGCCAGTACAGGCCCCCAGAGGGTCAGCTCACGAATGAGACTCCGCCTCTGTTCCAC | | |
| | TCTTACTCTGAAAACCATGATGGAGCCCAAGTAGAGGAACTTGATGACAGTGATTTCA | | |
| | GTGAAGATTCTTTTTCACACATCTCTAGTCAGAGGGCCACAAAGCAAAGGAACACCCT | | |
| | GGAGAATAGCGAAGACTCATTCTTCCTGCACCAGACGTGGGGACAGGGTCCTGAGAAG | | |
| | CAGGTGGCAGAAAGACAGCAGAGTCTGTTTTCTAGCCCCAGGACAGGTGACAAGAAAG | | |
| | ATCTAACTAAAAGCTGGGTGGACTCCAGGGACCCCATAAACCACAGAAGAGCAGCACT | | |
| | CGATCACAGCTGCAGCCCAAGTAAGGGGCCCGTGGACTGGAGCAGAGAGAACTCTACT | | |
| | TCCTCAGGGCCTTCTCCCAGAGACAGCCTGGCAGAGAACCCATACTGTTCACAGGTAG | | |
| | ATTTCATATATAGACAGGAAAGAGGTGGAGGCTCTTCCTTTGATCTCAGAAAGGATGC | | |
| | CTCCCAAAGTGAGGTTTCTGGGGAGAATGAGGGCAACTTGCCATCCCCAGAGGAAGAT | | |
| | GGTTTCACTATCTCATTGTCCCACGTTGCAGTTCCTGGATCCCCAGACCAAAGAGACA | | |
| | CAGTCACCACACCTCTGAGAGAAGTCAGTGCAGACGGCCCAATCCAGGTGACCAGCAC | | |
| | TGTGAAAAACGGTCATGCTGTCCCAGGAGAGGATCCTAGGGGGCAGTTAGGCACGCAT | | |
| | GCTGAATATGCTTCTGGACTCATGTCTCCCCTCACCATGTCCCTCCTGGAGAACCCAG | | |
| | ACAACGAAGGGTCTCCTCCCTCGGAGCAGCTGGTCCAGGATGGGGCTACGCACAGTCT | | |
| | AGTGGCAGAGAGCACAGGGGGCCCAGTTGTGAGCCACACAGTGCCATCTGGTGATCAA | | |
| | GAGGCAGCCTTGCCAGTGTCTTCAGCAACTAGGCACCTGTGGCTGTCCTCATCTCCCC | | |
| | CTGATAATAAGCCTGGTGGTGATCTTCCAGCTCTGTCCCCATCACCCATCCGTCAGCA | | |
| | CCCAGCTGACAAGCTGCCCAGCAGGGAGGCAGACCTAGGAGAGGCCTGCCAGAGCAGA | | |
| | GAGACTGTACTTTTCTCCCACGAACACATGGGTAGTGAGCAGTATGATGCTGATGCAG | | |
| | AGGAGACGGGGCTGGATGGCTCCTGGGGTTTCCCAGGAAAGCCCTTCACCACCATACA | | |
| | TATGGGGGTACCCCATTCTGGACCTACACTCACCCCACGAACAGGAAGTAGTGATGTG | | |
| | GCTGACCAGCTCTGGGCCCAGGAGAGAAAACATCCTACAAGGCTTGGTTGGCAGGAGT | | |
| | TTGGTTTGTCCACAGACCCCATCAAGTTGCCCTGCAACAGTGAAAATGTCACATGGCT | | |
| | CAAACCCAGGCCGATCTCAAGGCAGGTGGTCATCCGAGCACACCAGGAACAGCTGGAT | | |
| | GAAATGGCTGAGCTCGGCTTCAAGGAGGAGACGCTGATGAGCCAGCTGGCTTCTAATG | | |
| | ATTTTGAAGATTTTGTGACCCAGCTGGATGAAATCATGGTTCTGAAATCCAAGTGTAT | | |
| | CCAGAGTCTGAGGAGCCAGCTGCAGCTCTATCTCACCTGCCACGGGCCCACCGCAGCC | | |
| | CCTGAGGGAACAGTGCCGTCTTAGAGCCAGACCCT | | |
| | ORF Start: ATG at 10 | | ORF Stop: TAG at 4024 |
| | SEQ ID NO:328 | 1338 aa | MW at 148781.1 kD |

| | |
|---|---|
| NOV36a, | MASWLYECLCEAELAQYYSHFTALGLQKIDELAKITMKDYSKLGVHDMNDRKRLFQLI |
| CG157704-01 | |
| Protein Sequence | KIIKIMQEEDKAVSIPERHLQTSSLRIKSQELRSGPRRQLNFDSPADNKDRNASNDGF |
| | EMCSLSDFSANEQKSTYLKVLEHMLPDDSQYHTKTGILNATAGDSYVQTEISTSLFSP |
| | NYLSAILGDCDIPIIQRISHVSGYNYGIPHSCIRQNTSEKQNPWTEMEKIRVCVRKRP |
| | LGMREVRRGEINIITVEDKETLLVHEKKEAVDLTQYILQHVFYFDEVFGEACTNQDVY |
| | MKTTHPLIQHIFNGGNATCFAYGQTGAGKTYTMIGTHENPGLYALAAKDIFRQLEVSQ |
| | PRKHLFVWISFYEIYCGQLYDLLNRRKRLFAREDSKHMVQIVGLQELQVDSVELLLQV |
| | ILKGSKERSTGATGVNADSSRSHAVIQIQIKDSAKRTFGRISFIDLAGSERAADARDS |
| | DRQTKMEGAEINQSLLALKECIRALDQEHTHTPFRQSKLTQVLKDSPIGNAKTCMIAN |
| | ISPSHVATEHTLNTLRYADRVKELKKGIKCCTSVTSRNRTSGNSSPKRIQSSPGALSE |
| | DKCSPKKVKLGFQQSLTVAAPGSTRGKVHPLTSHPPNIPFTSAPKVSGKRGGSRGSPS |
| | QEWVIHASPVKGTVRSGHVAKKKPEESAPLCSEKNRMGNKTVLGWESRASGPGEGLVR |
| | GKLSTKCKKVQTVQPVQKQLVSRVELSFGNAHHRAEYSQDSQRGTPARPASEAWTNIP |
| | PHQKEREEHLRFYHQQFQQPPLLQQKLKYQPLKRSLRQYRPPEGQLTNETPPLFHSYS |
| | ENNDGAQVEELDDSDFSEDSFSHISSQRATKQRNTLENSEDSFFLHQTWGQGPEKQVA |
| | ERQQSLFSSPRTGDKKDLTKSWVDSRDPINHRRAALDHSCSPSKGPVDWSRENSTSSG |
| | PSPRDSLAEKPYCSQVDFIYRQERGGGSSFDLRKDASQSEVSGENEGNLPSPEEDGFT |
| | ISLSHVAVPGSPDQRDTVTTPLREVSADGPIQVTSTVKNGHAVPGEDPRGQLGTHAEY |
| | ASGLMSPLTMSLLENPDNEGSPPSEQLVQDGATHSLVAESTGGPVVSHTVPSGDQEAA |
| | LPVSSATRHLWLSSSPPDNKPGGDLPALSPSPIRQHPADKLPSREADLGEACQSRETV |
| | LFSHEHMGSEQYDADAEETGLDGSWGFPGKPFTTIHMGVPHSGPTLTPRTGSSDVADQ |
| | LWAQERKHPTRLGWQEFGLSTDPIKLPCNSENVTWLKPRPISRQVVIRAHQEQLDEMA |
| | ELGFKEETLMSQLASNDFEDFVTQLDEIMVLKSKCIQSLRSQLQLYLTCHGPTAAPEG |
| | TVPS |

Further analysis of the NOV36a protein yielded the following properties shown in Table 36B.

TABLE 36B
Protein Sequence Properties NOV36a

| | |
|---|---|
| PSort | 0.8200 probability located in nucleus; 0.3000 probability |
| analysis: | located in microbody (peroxisome); 0.1000 probability |
| | located in mitochondrial matrix space; 0.1000 probability |
| | located in lysosome (lumen) |
| SignalP | No Known Signal Sequence Predicted |
| analysis: | |

A search of the NOV36a protein against the Geneseq database, a proprietary database that contains sequences published in patents and patent publication, yielded several homologous proteins shown in Table 36C.

TABLE 36C
Geneseq Results for NOV36a

| | | | Identities/ | |
|---|---|---|---|---|
| | | | Similarities for | |
| Geneseq | Protein/Organism/Length | NOV36a Residues/ | the Matched | Expect |
| Identifier | [Patent #, Date] | Match Residues | Region | Value |
| AAU77182 | Human kinesin motor protein | 1 . . . 1338 | 1337/1368 (97%) | 0.0 |
| | KinI-3 - Homo sapiens, 1368 aa. | 1 . . . 1368 | 1338/1368 (97%) | |
| | [WO200226929-A2, 04 APR. | | | |
| | 2002] | | | |
| AAU77184 | Human KinI-3 DNA fragment | 195 . . . 566 | 371/372 (99%) | 0.0 |
| | with flanking vector sequences #2 - | 2 . . . 373 | 372/372 (99%) | |
| | Homo sapiens, 381 aa. | | | |
| | [WO200226929-A2, 04 APR. | | | |
| | 2002] | | | |
| AAU77183 | Human KinI-3 DNA fragment | 183 . . . 546 | 363/364 (99%) | 0.0 |
| | with flanking vector sequences #1 - | 2 . . . 365 | 364/364 (99%) | |
| | Homo sapiens, 373 aa. | | | |
| | [WO200226929-A2, 04 APR. | | | |
| | 2002] | | | |
| AAU77186 | Human KinI-3 DNA fragment | 213 . . . 566 | 353/354 (99%) | 0.0 |
| | with flanking vector sequences #4 - | 2 . . . 355 | 354/354 (99%) | |
| | Homo sapiens, 363 aa. | | | |
| | [WO200226929-A2, 04 APR. | | | |
| | 2002] | | | |
| AAU77185 | Human KinI-3 DNA fragment | 213 . . . 546 | 333/334 (99%) | 0.0 |
| | with flanking vector sequences #3 - | 2 . . . 335 | 334/334 (99%) | |
| | Homo sapiens, 343 aa. | | | |
| | [WO200226929-A2, 04 APR. | | | |
| | 2002] | | | |

In a BLAST search of public sequence datbases, the NOV36a protein was found to have homology to the proteins shown in the BLASTP data in Table 36D.

TABLE 36D
Public BLASTP Results for NOV36a

| | | | Identities/ | |
|---|---|---|---|---|
| Protein | | | Similarities for | |
| Accession | | NOV36a Residues/ | the Matched | Expect |
| Number | Protein/Organism/Length | Match Residues | Portion | Value |
| Q9GYC7 | Probable mitotic centromere | 1 . . . 548 | 222/551 (40%) | e−101 |
| | associated kinesin - Leishmania | 1 . . . 519 | 317/551 (57%) | |
| | major, 728 aa. | | | |
| Q9NV43 | OVARC1000605 protein - Homo | 37 . . . 208 | 172/172 (100%) | 5e−95 |
| | sapiens (Human), 172 aa. | 1 . . . 172 | 172/172 (100%) | |
| Q94GW1 | Kinesin-like protein - Oryza | 208 . . . 574 | 192/368 (52%) | 3e−94 |
| | sativa (Rice), 800 aa. | 188 . . . 539 | 251/368 (68%) | |
| P28740 | Kinesin-like protein KIF2 - Mus | 223 . . . 617 | 196/407 (48%) | 2e−93 |
| | musculus (Mouse), 716 aa. | 195 . . . 582 | 259/407 (63%) | |
| Q9VZ28 | CG1453 protein - Drosophila | 223 . . . 546 | 182/333 (54%) | 5e−93 |
| | melanogaster (Fruit fly), 803 aa. | 276 . . . 608 | 236/333 (70%) | |

PFam analysis predicts that the NOV36a protein contains the domains shown in the Table 36E.

TABLE 36E
Domain Analysis of NOV36a

| | | Identities/ | |
|---|---|---|---|
| | | Similarities for | |
| Pfam | NOV36a | the Matched | Expect |
| Domain | Match Region | Region | Value |
| SAM | 2 . . . 62 | 19/68 (28%) | 0.42 |
| | | 41/68 (60%) | |
| kinesin | 229 . . . 547 | 129/388 (33%) | 3.7e−89 |
| | | 236/388 (61%) | |

### Example 37

The NOV37 clone was analyzed, and the nucleotide and encoded polypeptide sequences are shown in Table 37A.

TABLE 37A
NOV37 Sequence Analysis

| | SEQ ID NO:329 | 2770 bp | |
|---|---|---|---|
| NOV37a, | TTATGGGACCATGATGTTGAGAGTTAGTGTGAAGTGGACCATTGAAAAAGCCAGCCAA | | |
| CG158218-01 | | | |
| DNA Sequence | AGTAGCATCTTCATCCGTTTCCAGGCCATGCCCTTTCATTATAACCAGAAGGCCCCAT | | |
| | GTTCTGAGTGCCATGATCTGATGTGTAGGAATGTCAATTCCACCCCGCAGATCATTGC | | |
| | AACTTTAGTGGACATACCTATACATGCCAAAAGCATCCTGCCTCCAGGGTCTGCACCT | | |
| | CTCTCTGCCCAACGGCTTTCTCTGAATGTCAGGGCACACAGGATTTATTCCATAGATG | | |
| | AAGATGAAAAATTAATACCTAGCTTGGAAATCATCTTACCACGTGATTTGGCAGATGG | | |
| | GTTTGTGAATAATAAGCGAGAAAGCTACAAATTTAAATTTCAAAGAATTTTTGATCAG | | |
| | GATGCAAACCAAGAGACCGTTTTTGAAAACATTGCCAAACCAGTTGCTGGGAGGTATC | | |
| | TCACCCCTGGTGGTAAGGATGTCCTGGCAGGTTACAATGGTACCATCTTTGCATATGG | | |
| | GCAAACAGGCAGCGGGAAGACATTCACTATCACAGGGGGTGCAGAGCGTTACAGTGAC | | |
| | ACAGGCATTATCCCAAGGACACTGTCATACATTTTTGAACAGTTACAAAAGGACAGCA | | |
| | GCAAAATATATACAACACACATTTCCTATTTGGAAATCTACAATGAATGTGGTTATGA | | |
| | TCTTTTGGATCCAAGACATGAAGCCTCCAGTTTGGAAGATTTGCCGAAAGTGACAATA | | |
| | CTGGAGGATCCTGATCAGAACATTCACCTGAAAAACTTGACTCTCCATCAGGCAACCA | | |
| | CAGAGGAAGAAGCTCTGAATTTGCTTTTTTTAGGAGACACCAACCGAATGATTGCAGA | | |
| | GACTCCTATGAACCAAGCTTCAACCCGTTCCCACTGCATTTTCACCATTCATTTGTCA | | |
| | AGCAAGGAACCAGGATCTGCAACTGTACGACATGCCAAACTCCATCTGGTTGACCTGG | | |
| | CTGGTTCAGAGCGAGTTGCAAAGACTGGAGTAGGGGGCCATCTTCTAACAGAGGCCAA | | |
| | GTATATCAACTTGTCACTACATTACTTAGAACAGGTTATCATTGCCCTTTCAGAAAAG | | |
| | CACCGTTCGCACATTCCTTATAGAAACTCCATCATGACCAGTGTCCTAAGAGACAGTT | | |
| | TGGGAGGGAACTGCATGACAACTATGATTGCAACACTCTCCTTGGAGAAAAGGAATCT | | |
| | TGATGAGTCTATATCAACCTGCAGATTTGCACAGCGAGTGGCACTCATAAAGAATGAA | | |
| | GCTGTTCTTAATGAAGAAATTAACCCCAGATTAGTGATTAAACGCCTACAAAAGGAAA | | |
| | TCCAGGAACTGAAGGATGAACTGGCCATGGTCACTGGGGAGCAGAGGACAGAGGCACT | | |
| | CACAGAAGCAGAGCTCCTTCAGCTGGAAAAACTAATAACATCCTTTTTGGAAGACCAG | | |
| | GATTCAGACAGTAGATTAGAGGTTGGCGCGGATATGCGTAAAGTTCATCACTGTTTTC | | |
| | ATCATTTAAAGAAACTATTGAATGACAAGAAGATCCTTGAAAACAATACAGTCTCCTC | | |
| | TGAAAGCAAAGACCAAGATTGTCAAGAACCATTAAAAGAAGAAGAATATAGAAAGCTA | | |
| | CGAGATATTCTGAAACAGAGAGATAACGAAATCAATATCCTGGTCAACATGTTAAAAA | | |
| | AAGAAAAGAAGAAAGCTCAGGAGGCTCTCCACTTGGCTGGCATGGATAGACGTGAATT | | |
| | CAGACAGTCCCAGAGCCCACCCTTCCGCCTAGGAAACCCAGAAGAAGGTCAAAGAATG | | |
| | CGACTATCCTCAGCTCCCTCACAGGCCCAGGACTTCAGCATTTTGGGGAAAAGATCCA | | |
| | GTTTGCTCCACAAGAAAATAGGAATGAGAGAGGAAATGTCATTAGGATGCCAGGAGGC | | |
| | TTTTGAAATCTTCAAGACGGACCACGCTGACAGCGTTACCATCGATGACAACAAACAG | | |
| | ATTCTGAAACAGAGATTTTCTGAAGCCAAGGCCCTGGGAGAAAGTATAAATGAAGCAA | | |
| | GAAGTAAAATTGGTCACCTGAAGGAAGAAATCACCCAGCGGCATATACAGCAAGTAGC | | |
| | CCTAGGAATCTCGGAAAACATGGCCGTGCCTCTGATGCCAGACCAGCAGGAGGAGAAG | | |
| | CTGCGATCACAACTGGAGGAAGAAAAGAGAAGGTATAAAACAATGTTCACTCGCCTGA | | |
| | AAGCCCTGAAGGTGGAGATCGAGCACTTGCAGCTGCTCATGGACAAAGCCAAGGTGAA | | |
| | GCTACAGAAAGAGTTTGAAGTCTGGTGGGCAGAGGAGGCCACCAACCTGCAGGTAAAT | | |
| | TCTCCAGCAGTGAATTCACTCGATCACACGAAGCCATTTCTCCAGACATCTGACTCCC | | |
| | AGCATGAATGGTCCCAACTCCTCTCTAACAAAAGTTCTGCAGGCTGGGAAGTCCAAGA | | |
| | TCAAGGCACTGGCAGATTCGATGTCTGTGATGTGAATGCCAGGAAAATCCTGCCCTCG | | |
| | CCTTGCCCCAGTCCACACACCCAGAAACAGAGCAGCACCAGCACCCCACTGGAAGACA | | |
| | GCATCCCCAAGAGGCCAGTGTCGTCCATCCCTCTCACCGGAGACAGCCAGACGGACTC | | |
| | GGACATCATCGCCTTCATCAAGGCCAGACAGAGCATTCTGCAGAAGCAATATCTTCAG | | |
| | CTCCTTTGTTCTCTGTTCCCAAAGTCAGCTGTCTCCTCTGCTCAGGCTTCTACAAACA | | |
| | GGAAGGGGCTGAGTGATGTTTTGGTAACTCGTTGAACCCCTGGC | | |
| | ORF Start: ATG at 11 | | ORF Stop: TGA at 2759 |
| | SEQ ID NO:330 | 916 aa | MW at 103840.1 kD |

| | |
|---|---|
| NOV37a, | MMLRVSVKWTIEKASQSSIFIRFQAMPFHYNQKAPCSECHDLMCRNVNSTPQIIATLV |
| CG158218-01 | |
| Protein Sequence | DIPIHAKSILPPGSAPLSAQRLSLNVRAHRIYSIDEDEKLIPSLEIILPRDLADGFVN |
| | NKRESYKFKFQRIFDQDANQETVFENIAKPVAGRYLTPGGKDVLAGYNGTIFAYGQTG |
| | SGKTFTITGGAERYSDRGIIPRTLSYIFEQLQKDSSKIYTTHISYLEIYNECGYDLLD |
| | PRHEASSLEDLPKVTILEDPDQNIHLKNLTLHQATTEEEALNLLFLGDTNRMIAETPM |
| | NQASTRSHCIFTIHLSSKEPGSATVRHAKLHLVDLAGSERVAKTGVGGHLLTEAKYIN |
| | LSLHYLEQVIIALSEKHRSHIPYRNSMMTSVLRDSLGGNCMTTMIATLSLEKRNLDES |
| | ISTCRFAQRVALIKNEAVLNEEINPRLVIKRLQKEIQELKDELAMVTGEQRTEALTEA |
| | ELLQLEKLITSFLEDQDSDSRLEVGADMRKVHHCFHHLKKLLNDKKTLENNTVSSESK |
| | DQDCQEPLKEEEYRKLRDTLKQRDNEINILVNMLKKEKKKAQEALHLAGMDRREPRQS |
| | QSPPFRLGNPEEGQRMRLSSAPSQAQDFSILGKRSSLLHKKIGMREEMSLGCQEAFEI |
| | FKRDHADSVTIDDNKQILKQRFSEAKALCESINEARSKIGHLKEEITQRHIQQVALGI |
| | SENMAVPLMPDQQEEKLRSQLEEEKRRYKTMFTRLKALKVEIEHLQLLMDKAKVKLQK |
| | EFEVWWAEEATNLQVNSPAVNSLDHTKPFLQTSDSQHEWSQLLSNKSSGGWEVQDQGT |
| | GRFDVCDVNARKILPSPCPSPHSQKQSSTSTPLEDSIPKRPVSSIPLTGDSQTDSDII |
| | AFIKARQSILQKQYLQLLCSLFPKSAVSSAQASTNRKGLSDVLVTR |

Further analysis of the NOV37a protein yielded the following properties shown in Table 37B.

TABLE 37B
Protein Sequence Properties NOV37a

| | |
|---|---|
| PSort | 0.6863 probability located in mitochondrial matrix space; |
| analysis: | 0.3737 probability located in mitochondrial inner membrane; |
| | 0.3737 probability located in mitochondrial intermembrane |
| | space; 0.3737 probability located in mitochondrial outer |
| | membrane |
| SignalP | No Known Signal Sequence Predicted |
| analysis: | |

A search of the NOV37a protein against the Geneseq database, a proprietary database that contains sequences published in patents and patent publication, yielded several homologous proteins shown in Table 37C.

TABLE 37C
Geneseq Results for NOV37a

| | | | Identities/ | |
|---|---|---|---|---|
| | | | Similarities for | |
| Geneseq | Protein/Organism/Length | NOV37a Residues/ | the Matched | Expect |
| Identifier | [Patent #, Date] | Match Residues | Region | Value |
| AAU75177 | Human kinesin protein 9 - Homo | 86 . . . 762 | 220/685 (32%) | 3e−91 |
| | sapiens, 725 aa. [CN1319665-A, | 20 . . . 643 | 363/685 (52%) | |
| | 31 OCT. 2001] | | | |
| AAE14609 | Human microtubule motor protein | 159 . . . 322 | 164/164 (100%) | 3e−91 |
| | HsKif6 motor domain - Homo | 28 . . . 191 | 164/164 (100%) | |
| | sapiens, 205 aa. [US6346410-B1, | | | |
| | 12 FEB. 2002] | | | |
| AAU75800 | Human ortholog of mouse kinesin | 86 . . . 762 | 217/739 (29%) | 8e−81 |
| | Kif9, HsKif9 - Homo sapiens, 790 | 20 . . . 708 | 362/739 (48%) | |
| | aa. [US6331430-B1, 18 DEC. | | | |
| | 2001] | | | |
| ABB80741 | Human kinesin motor protein, | 86 . . . 762 | 217/739 (29%) | 8e−81 |
| | HsKif9 sequence - Homo sapiens, | 20 . . . 708 | 362/739 (48%) | |
| | 790 aa. [US6355447-B1, 12 MAR. | | | |
| | 2002] | | | |
| AAB94768 | Human protein sequence SEQ ID | 86 . . . 510 | 162/432 (37%) | 1e−77 |
| | NO: 15849 - Homo sapiens, 664 aa. | 20 . . . 433 | 258/432 (59%) | |
| | [EP1074617-A2, 07 FEB. 2001] | | | |

In a BLAST search of public sequence datbases, the NOV37a protein was found to have homology to the proteins shown in the BLASTP data in Table 37D.

TABLE 37D
Public BLASTP Results for NOV37a

| | | | Identities/ | |
|---|---|---|---|---|
| Protein | | | Similarities for | |
| Accession | | NOV37a Residues/ | the Matched | Expect |
| Number | Protein/Organism/Length | Match Residues | Portion | Value |
| 054720 | Kinesin-related protein 3A - | 81 . . . 560 | 416/480 (86%) | 0.0 |
| | Rattus norvegicus (Rat), 486 aa | 15 . . . 486 | 442/480 (91%) | |
| | (fragment). | | | |
| Q8R471 | Kinesin-related protein 3B - | 81 . . . 507 | 376/427 (88%) | 0.0 |
| | Rattus norvegicus (Rat), 452 aa | 14 . . . 432 | 396/427 (92%) | |
| | (fragment). | | | |
| Q8WTV4 | Hypothetical 30.1 kDa protein - | 624 . . . 885 | 261/262 (99%) | e−147 |
| | Homo sapiens (Human), 265 aa. | 1 . . . 262 | 261/262 (99%) | |
| Q9UJR0 | DJ1043E3.1 (Novel protein) - | 434 . . . 622 | 189/189 (100%) | e−102 |
| | Homo sapiens (Human), 189 aa | 1 . . . 189 | 189/189 (100%) | |
| | (fragment). | | | |
| O35067 | Motor domain of KIF6 - Mus | 167 . . . 329 | 155/165 (93%) | 2e−84 |
| | musculus (Mouse), 165 aa | 1 . . . 165 | 158/165 (94%) | |
| | (fragment). | | | |

PFam analysis predicts that the NOV37a protein contains the domains shown in the Table 37E.

TABLE 37E
Domain Analysis of NOV37a

| | | Identities/ | |
|---|---|---|---|
| | | Similarities for | |
| Pfam | NOV37a | the Matched | Expect |
| Domain | Match Region | Region | Value |
| kinesin | 124 . . . 449 | 153/375 (41%) | 6.5e−119 |
| | | 255/375 (68%) | |

### Example 38

The NOV38 clone was analyzed, and the nucleotide and encoded polypeptide sequences are shown in Table 38A.

TABLE 38A
NOV38 Sequence Analysis

| | SEQ ID NO:331 | 1184 bp | |
|---|---|---|---|
| NOV38a, | AGTTCCTCCTAACTCCTGCCAGAAACAGCTCTCCTCAACATGAGAGCTGCACCCCTCC | | |
| CG158513-01 | | | |
| DNA Sequence | TCCTGGCCAGGGCAGCAAGCCTTAGCCTTGGCTTCTTGTTTCTGCTTTTTTTCTGGCT | | |
| | AGACCGAAGTGTACTAGCCAAGGAGTTGAAGTTTGTGACTTTGGTGTTTCGGCATGGA | | |
| | GACCGAAGTCCCATTGACACCTTTCCCACTGACCCCATAAAGGAATCCTCATGGCCAC | | |
| | AAGGATTTGGCCAACTCACCCAGCTGGGCATGGAGCAGCATTATGAACTTGGAGAGTA | | |
| | TATAAGAAAGAGATATAGAAAATTCTTGAATGAGTCCTATAAACATGAACAGGTTTAT | | |
| | ATTCGAAGCACAGACGTTGACCGGACTTTGATGAGTGCTATGACAAACCTGGCAGCCC | | |
| | TGTTTCCCCCAGAAGGTGTCAGCATCTGGAATCCTATCCTACTCTGGCAGCCCATCCC | | |
| | GGTGCACACAGTTCCTCTTTCTGAAGATCAGGATTTTATAGCTACCTTGGGAAAACTT | | |
| | TCAGGATTACATGGCCAGGACCTTTTTGGAATTTGGAGTAAAGTCTACGACCCTTTAT | | |
| | ATTGTGAGAGTGTTCACAATTTCACTTTACCCTCCTGGGCCACTGAGGACACCATGAC | | |
| | TAAGTTGAGAGAATTGTCAGAATTGTCCCTCCTGTCCCTCTATGGAATTCACAAGCAG | | |
| | AAAGAGAAATCTAGGCTCCAAGGGGGTGTCCTGGTCAATGAAATCCTCAATCACATGA | | |
| | AGAGAGCAACTCAGATACCAAGCTACAAAAAACTCATCATGTATTCTGCGCATGACAC | | |
| | TACTGTGAGTGGCCTACAGATGGCGCTAGATGTTTACAACGGACTCCTTCCTCCCTAT | | |
| | GCTTCTTGCCACTTGACGGAATTGTACTTTGAGAAGGGGGAGTACTTTGTGGAGATGT | | |
| | ACTATCGGAATGAGACGCAGCACGAGCCGTATCCCCTCATGCTACCTGGCTGCAGCCC | | |
| | CAGCTGTCCTCTGGAGAGGTTTGCTGAGCTGGTTGGCCCTGTGATCCCTCAAGACTGG | | |
| | TCCACGGAGTGTATGACCACAAACAGCCATCAAGGTACTGAGGACAGTACAGATTAGT | | |
| | GTGCACAGAGATCTCTGTAGAAAGAGTAGCTGCCCTTTCTCAGGGCAGATGATGCTTT | | |
| | GAGAACATACTTTGGCCATTACCC | | |
| | ORF Start: ATG at 40 | | ORF Stop: TAG at 1099 |
| | SEQ ID NO:332 | 353 aa | MW at 404492.9 kD |
| NOV38a, | MRAAPLLLARAASLSLGFLFLLFFWLDRSVLAKELKFVTLVFRHGDRSPIDTFPTDPI | | |
| CG158513-01 | | | |
| Protein Sequence | KESSWPQGFGQLTQLGMEQHYELGEYIRKRYRKFLNESYKHEQVYIRSTDVDRTLMSA | | |
| | MTNLAALFPPEGVSIWNPILLWQPIPVHTVPLSEDQDFIATLGKLSGLHGQDLFGIWS | | |
| | KVYDPLYCESVHNFTLPSWATEDTMTKLRELSELSLLSLYGIHKQKEKSRLQGGVLVN | | |
| | EILNHMKRATQIPSYKKLIMYSAHDTTVSCLQMALDVYNGLLPPYASCHLTELYFEKG | | |
| | EYFVEMYYRNETQHEPYPLMLPGCSPSCPLERFAELVGPVIPQDWSTECMTTNSHQGT | | |
| | EDSTD | | |
| | SEQ ID NO:333 | 1184 bp | |
| NOV38b, | AGTTCCTCCTAACTCCTGCCAGAAACAGCTCTCCTCAACATGAGAGCTGCACCCCTCC | | |
| CG158513-02 | | | |
| DNA Sequence | TCCTGGCCAGGGCAGCAAGCCTTAGCCTTGGCTTCTTGTTTCTGCTTTTTTTCTGGCT | | |
| | AGACCGAAGTGTACTAGCCAAGGAGTTGAAGTTTGTGACTTTGGTGTTTCGGCATGGA | | |
| | GACCGAAGTCCCATTGACACCTTTCCCACTGACCCCATAAAGGAATCCTCATGGCCAC | | |
| | AAGGATTTCGCCAACTCACCCAGCTGGGCATGGAGCAGCATTATGAACTTGGAGAGTA | | |
| | TATAAGAAAGAGATATAGAAAATTCTTGAATGAGTCCTATAAACATGAACAGGTTTAT | | |
| | ATTCGAAGCACAGACGTTGACCGGACTTTGATGAGTGCTATGACAAACCTGGCAGCCC | | |
| | TGTTTCCCCCAGAAGGTGTCAGCATCTGGAATCCTATCCTACTCTGGCAGCCCATCCC | | |
| | GGTGCACACAGTTCCTCTTTCTGAAGATCAGGATTTTATAGCTACCTTGGGAAAACTT | | |
| | TCAGGATTACATGGCCAGGACCTTTTTGGAATTTGGAGTAAAGTCTACGACCCTTTAT | | |
| | ATTGTGAGAGTGTTCACAATTTCACTTTACCCTCCTGGGCCACTGAGGACACCATGAC | | |
| | TAAGTTGAGAGAATTGTCAGAATTGTCCCTCCTGTCCCTCTATGGAATTCACAAGCAG | | |
| | AAAGAGAAATCTAGGCTCCAAGGGGGTGTCCTGGTCAATGAAATCCTCAATCACATGA | | |
| | AGAGAGCAACTCAGATACCAAGCTACAAAAAACTTATCATGTATTCTGCGCATGACAC | | |
| | TACTGTGAGTGGTCTACAGATGGCGCTAGATGTTTACAACGGACTCCTTCCTCCCTAT | | |
| | GCTTCTTGCCACTTGACGGAATTGTACTTTGAGAAGGGGGAGTACTTTGTGGAGATGT | | |
| | ACTACCGGAATGAGACGCAGCACGAGCCGTATCCCCTCATGCTACCTGGCTGCAGCCC | | |
| | CAGCTGTCCTCTGGAGAGGTTTGCTGAGCTGGTTGGCCCTGTGATCCCTCAAGACTGG | | |
| | TCCACGGAGTGTATGACCACAAACAGCCATCAAGGTACTGAGGACAGTACAGATTAGT | | |
| | GTGCACAGAGATCTCTGTAGAAAGAGTAGCTGCCCTTTCTCAGGGCAGATGATGCTTT | | |
| | GAGAACATACTTTGGCCATTACCC | | |
| | ORF Start: ATG at 40 | | ORF Stop: TAG at 1099 |
| | SEQ ID NO:334 | 353 aa | MW at 40442.9 kD |

| | |
|---|---|
| NOV38b, | MRAAPLLLARAASLSLGFLFLLFFWLDRSVLAKELKFVTLVFRHGDRSPIDTFPTDPI |
| CG158513-02 | |
| Protein Sequence | KESSWPQGFGQLTQLGMEQHYELGEYIRKRYRKFLNESYKHEQVYIRSTDVDRTLMSA |
| | MTNLAALFPPEGVSIWNPILLWQPIPVHTVPLSEDQDFIATLGKLSGLHGQDLFGIWS |
| | KVYDPLYCESVHNFTLPSWATEDTMTKLRELSELSLLSLYGIHKQKEKSRLQGGVLVN |
| | EILNHMKRATQIPSYKKLIMYSAHDTTVSGLQMALDVYNGLLPPYASCHLTELYFEKG |
| | EYFVEMYYRNETQHEPYPLMLPGCSPSCPLERFAELVGPVIPQDWSTECMTTNSHQGT |
| | EDSTD |

Sequence comparison of the above protein sequences yields the following sequence relationships shown in Table 38B.

TABLE 38B
Comparison of NOV38a against NOV38b.

| | | Identities/ |
|---|---|---|
| | | Similarities for |
| Protein | NOV38a Residues/ | the Matched |
| Sequence | Match Residues | Region |
| NOV38b | 1 . . . 353 | 353/353 (100%) |
| | 1 . . . 353 | 353/353 (100%) |

Further analysis of the NOV38a protein yielded the following properties shown in Table 38C.

TABLE 38C
Protein Sequence Properties NOV38a

| | |
|---|---|
| PSort | 0.4600 probability located in plasma membrane; 0.2083 |
| analysis: | probability located in microbody (peroxisome); 0.1000 |
| | probability located in endoplasmic reticulum (membrane); |
| | 0.1000 probability located in endoplasmic reticulum (lumen) |
| SignalP | Cleavage site between residues 33 and 34 |
| analysis: | |

A search of the NOV38a protein against the Geneseq database, a proprietary database that contains sequences published in patents and patent publication, yielded several homologous proteins shown in Table 38D.

TABLE 38D
Geneseq Results for NOV38a

| | | | Identities/ | |
|---|---|---|---|---|
| | | | Similarities for | |
| Geneseq | Protein/Organism/Length | NOV38a Residues/ | the Matched | Expect |
| Identifier | [Patent #, Date] | Match Residues | Region | Value |
| AAB74820 | Prostate tumour antigen amino acid | 1 . . . 353 | 353/386 (91%) | 0.0 |
| | sequence for PAP - Homo sapiens, | 1 . . . 386 | 353/386 (91%) | |
| | 386 aa. [WO200125272-A2, 12 | | | |
| | APR. 2001] | | | |
| AAG62145 | Human prostatic acid phosphatase | 1 . . . 353 | 353/386 (91%) | 0.0 |
| | SEQ ID NO: 328 - Homo sapiens, | 1 . . . 386 | 353/386 (91%) | |
| | 386 aa. [WO200125273-A2, 12 | | | |
| | APR. 2001] | | | |
| AAU02172 | Biomarker UC band 47 (PAP), used | 1 . . . 353 | 353/386 (91%) | 0.0 |
| | in diagnosis and prognosis of | 1 . . . 386 | 353/386 (91%) | |
| | cancer - Homo sapiens, 386 aa. | | | |
| | [US6218529-B1, 17 APR. 2001] | | | |
| AAU06277 | Prostatic Acid Phosphatase (PAP) | 1 . . . 353 | 353/386 (91%) | 0.0 |
| | polypeptide - Homo sapiens, 386 aa. | 1 . . . 386 | 353/386 (91%) | |
| | [WO200145728-A2, 28 JUN. 2001] | | | |
| AAY59293 | Prostatic acid phosphatase marker | 1 . . . 353 | 353/386 (91%) | 0.0 |
| | UC Band #47 amino acid sequence - | 1 . . . 386 | 353/386 (91%) | |
| | Homo sapiens, 386 aa. | | | |
| | [WO9964631-A1, 16 DEC. 1999] | | | |

In a BLAST search of public sequence datbases, the NOV38a protein was found to have homology to the proteins shown in the BLASTP data in Table 38E.

TABLE 38E
Public BLASTP Results for NOV38a

| | | | Identities/ | |
|---|---|---|---|---|
| Protein | | | Similarities for | |
| Accession | | NOV38a Residues/ | the Matched | Expect |
| Number | Protein/Organism/Length | Match Residues | Portion | Value |
| P15309 | Prostatic acid phosphatase | 1 . . . 353 | 353/386 (91%) | 0.0 |
| | precursor (EC 3.1.3.2) - Homo | 1 . . . 386 | 353/386 (91%) | |
| | sapiens (Human), 386 aa. | | | |
| Q96KY0 | Acid phosphatase, prostate - | 1 . . . 353 | 352/386 (91%) | 0.0 |
| | Homo sapiens (Human), 386 aa. | 1 . . . 386 | 353/386 (91%) | |
| Q96QK9 | Acid phosphatase, prostate - | 1 . . . 353 | 350/386 (90%) | 0.0 |
| | Homo sapiens (Human), 386 aa. | 1 . . . 386 | 351/386 (90%) | |
| Q96QM0 | Acid phosphatase, prostate - | 1 . . . 346 | 345/379 (91%) | 0.0 |
| | Homo sapiens (Human), 418 aa. | 1 . . . 379 | 345/379 (91%) | |
| Q9QXH7 | Prostatic acid phosphatase - | 1 . . . 347 | 281/380 (73%) | e−162 |
| | Mus musculus (Mouse), 381 aa. | 1 . . . 379 | 307/380 (79%) | |

PFam analysis predicts that the NOV38a protein contains the domains shown in the Table 38F.

TABLE 38F
Domain Analysis of NOV38a

| | | Identities/ | |
|---|---|---|---|
| | | Similarities for | |
| Pfam | NOV38a | the Matched | Expect |
| Domain | Match Region | Region | Value |
| Acid_phosphat | 33 . . . 340 | 128/436 (29%) | 2.7e−126 |
| | | 300/436 (69%) | |

### Example 39

The NOV39 clone was analyzed, and the nucleotide and encoded polypeptide sequences are shown in Table 39A.

TABLE 39A
NOV39 Sequence Analysis

| | SEQ ID NO:335 | 1967 bp | |
|---|---|---|---|
| NOV39a, | GGAGCCATGGCCCTGAGCGAGCTGGCGCTGGTCCGCTGGCTGCAGGAGAGCCGCCGCT | | |
| CG158583-01 | | | |
| DNA Sequence | CGCGGAAGCTCATCCTGTTCATCGTGTTCCTGGCGCTGCTGCTGGACAACATGCTGCT | | |
| | CACTGTCGTGGTAGAGAGAGGGTTTCTCCATGTTGGCCAGCCTGGTCTCGAACTCCTG | | |
| | ACCTCAGGTGATCCACCTGCCTCAGCTTCCCAAAGTCCTGGAATTACAGTCCCCATCA | | |
| | TCCCAAGTTATCTGTACAGCATTAAGCATGAGAAGAATGCTACAGAAATCCAGACGGC | | |
| | CAGGCCAGTGCACACTGCCTCCATCTCAGACAGCTTCCAGAGCATCTTCTCCTATTAT | | |
| | GATAACTCGACTATGGTCACCGGGAATGCTACCAGAGACCTGACACTTCATCAGACCG | | |
| | CCACACAGCACATGGTGACCAACGCGTCCGCTGTTCCTTCCGACTGTCCCAGTGAAGA | | |
| | CAAAGACCTCCTGAATGAAAACGTGCAAGTTGGTCTGTTGTTTGCCTCGAAAGCCACC | | |
| | GTCCAGCTCATCACCAACCCTTTCATAGGACTACTGACCAACAGAATTGGCTATCCAA | | |
| | TTCCCATATTTGCGGGATTCTGCATCATGTTTGTCTCAACAATTATGTTTGCCTTCTC | | |
| | CAGCAGCTATGCCTTCCTGCTGATTGCCAGGTCGCTGCAGGGCATCGGCTCGTCCTGC | | |
| | TCCTCTGTGGCTGGGATGGGCATGCTTGCCAGTGTCTACACAGATGATGAAGAGAGAG | | |
| | GCAACGTCATGGGAATCGCCTTGGGAGGCCTGGCCATGGGGGTCTTAGTGGCCCCCCC | | |
| | CTTCGGGAGTGTGCTCTATGAGTTTGTGGGGAAGACGGCTCCGTTCCTGGTGCTGGCC | | |
| | GCCCTCGTACTCTTGGATGGAGCTATTCAGCTCTTTGTGCTCCAGCCGTCCCGGGTGC | | |
| | AGCCAGAGAGTCAGAAGGGGACACCCCTAACCACGCTGCTGAAGGACCCGTACATCCT | | |
| | CATTGCTGCAGGCTCCATCTGCTTTGCAAACATGGGCATCGCCATGCTGGAGCCAGCC | | |
| | CTGCCCATCTCGATCATGGAGACCATGTGTTCCCGAAAGTGGCAGCTGGGCGTTGCCT | | |
| | TCTTGCCAGCTAGTATCTCTTATCTCATTGGAACCAATATTTTTGGGATACTTGCACA | | |
| | CAAAATGGGGAGGTGGCTTTGTGCTCTTCTGGGAATGATAATTGTTGGAGTCAGCATT | | |
| | TTATGTATTCCATTTGCAAAAAACATTTATGGACTCATAGCTCCGAACTTTGGAGTTG | | |
| | GTTTTGCAATTGGAATGGTGGATTCGTCAATGATGCCTATCATGGGCTACCTCGTAGA | | |
| | CCTGCGGCACGTGTCCGTCTATGGGAGTGTGTACGCCATTGCGGATGTGGCATTTTGT | | |
| | ATGGGGTATGCTATAGGTCCTTCTGCTGGTGGTGCTATTGCAAAGGCAATTGGATTTC | | |
| | CATGGCTCATGACAATTATTGGGATAATTGATATTCTTTTTGCCCCTCTCTGCTTTTT | | |
| | TCTTCGAAGTCCACCTGCCAAAGAAGAAAAAATGGCTATTCTCATGGATCACAACTGC | | |
| | CCTATTAAAACAAAAATGTACACTCAGAATAATATCCAGTCATATCCGATAGGTGAAG | | |
| | ATGAAGAATCTGAAAGTGACTGAGATGAGATCCTCAAAATCATCAAAGTGTTTAATT | | |
| | GTATAAAACAGTGTTTCCAGTGACACAACTCATCCAGAACTGTCTTAGTCATACCATC | | |
| | CATCCCTGGTGAAAGAGTAAAACCAAAGGTTATTATTTCCTTTCCATGGTTATGGTCG | | |
| | ATTGCCAACAGCCTTATAAACAAAAAGAAGCTTTTCTAGGGGTTTGTATAAATAGTGT | | |
| | TGAAACTTTATTTTATGTATTTAATTTTATTAAATATCATACAATATATTTTGATGAA | | |
| | ATAGGTATTGTGTAAATCTATAAATATTTGAATCCAAACCAAATATAATTTCC | | |
| | ORF Start: ATG at 7 | | ORF Stop: TGA at 1645 |
| | SEQ ID NO:336 | 546 aa | MW at 58912.5 kD |
| NOV39a, | MALSELALVRWLQESRRSRKLILFIVFLALLLDNMLLTVVVERGFLHVGQPGLELLTS | | |
| CG158583-01 | | | |
| Protein Sequence | GDPPASASQSPGITVPIIPSYLYSIKHEKNATEIQTARPVHTASISDSFQSIFSYYDN | | |
| | STMVTGNATRDLTLHQTATQHMVTNASAVPSDCPSEDKDLLNENVQVGLLFASKATVQ | | |
| | LITNPFIGLLTNRIGYPIPIFAGFCIMFVSTIMFAFSSSYAFLLIARSLQGIGSSCSS | | |
| | VAGMGMLASVYTDDEERGNVMGIALGGLAMGVLVGPPFGSVLYEFVGKTAPFLVLAAL | | |
| | VLLDGAIQLFVLQPSRVQPESQKGTPLTTLLKDPYILIAAGSICFANMGIAMLEPALP | | |
| | IWMMETMCSRKWQLGVAFLPASISYLIGTNIFGILAHKMGRWLCALLGMIIVGVSILC | | |
| | IPFAKNIYGLIAPNFGVGFAIGMVDSSMMPIMGYLVDLRHVSVYGSVYAIADVAFCMG | | |
| | YAIGPSAGGAIAKAIGFPWLMTIIGIIDILFAPLCFFLRSPPAKEEKMAILMDHNCPI | | |
| | KTKMYTQNNIQSYPIGEDEESESD | | |
| | SEQ ID NO:337 | 1952 bp | |
| NOV39b, | GCAGGCATCGCAAGCGACCCCGAGCGGAGCCCCGGAGCCATGGCCCTGAGCGAGCTGG | | |
| CG158583-02 | | | |
| DNA Sequence | CGCTGGTCCGCTGGCTGCAGGAGAGCCGCCGCTCGCGGAAGCTCATCCTGTTCATCGT | | |
| | GTTCCTGGCGCTGCTGCTGGACAACATGCTGCTCACTGTCGTGGGTTCAAGCGATCCT | | |
| | CCTTTCTCAGCCTCCAAAGGACCTGGGATTACAGTCCCCATCATCCCAAGTTATCTGT | | |
| | ACAGCATTAAGCATGAGAAGAATGCTACAGAAATCCAGACGGCCAGGCCAGTGCACAC | | |
| | TGCCTCCATCTCAGACAGCTTCCAGAGCATCTTCTCCTATTATGATAACTCGACTATG | | |
| | GTCACCGGGAATGCTACCAGAGACCTGACACTTCATCAGACCGCCACACAGCACATGG | | |
| | TGACCAACGCGTCCGCTGTTCCTTCCGACTGTCCCAGTGAAGACAAAGACCTCCTGAA | | |
| | TGAAAACGTGCAAGTTGGTCTGTTGTTTGCCTCGAAAGCCACCGTCCAGCTCATCACC | | |
| | AACCCTTTCATAGGACTACTGACCAACAGAATTGGCTATCCAATTCCCATATTTGCGG | | |
| | GATTCTGCATACATGTTGTCTCAACAATTATGTTTGCCTTCTCCAGCAGCTATGCCTT | | |
| | CCTGCTGATTGCCAGGTCGCTGCAGGCCATCGGCTCGTCCTGCTCCTCTGTGGCTGGG | | |
| | ATGGGCATGCTTGCCAGTGTCTACACAGATGATGAAGAGAGAGGCAACGTCATGGGAA | | |
| | TCGCCTTGGGAGGCCTGGCCATGGGGGTCTTAGTGGGCCCCCCCTTCGGGAGTGTGCT | | |
| | CTATGAGTTTGTGGGGAAGACGGCTCCGTTCCTGGTGCTGGCCGCCCTGGTACTCTTG | | |
| | GATGGAGCTATTCAGCTCTTTGTGCTCCAGCCGTCCCGGGTGCAGCCAGAGAGTCAGA | | |
| | AGGGGACACCCCTAACCACGCTGCTGAAGGACCCGTACATCCTCATTGCTGCAGGCTC | | |
| | CATCTGCTTTGCAAACATGGGCATCGCCATGCTGGAGCCACCCCTGCCCATCTGGATG | | |
| | ATGGAGACCATGTGTTCCCGAAAGTGGCAGCTGGGCGTTGCCTTCTTGCCAGCTAGTA | | |
| | TCTCTTATCTCATTGGAACCAATATTTTTGGGATACTTGCACACAAAATGGGGAGGTG | | |
| | GCTTTGTGCTCTTCTGGGAATGATAATTGTTGGAGTCAGCATTTTATGTATTCCATTT | | |
| | GCAAAAAACATTTATGGACTCATAGCTCCGAACTTTGGAGTTGGTTTTGCAATTGGAA | | |
| | TGGTGGATTCGTCAATGATGCCTATCATGGGCTACCTCGTAGACCTGCGGCACGTGTC | | |
| | CGTCTATGGGAGTGTGTACGCCATTGCGGATGTGGCATTTTGTATGGGGTATGCTATA | | |
| | GGTCCTTCTGCTGGTGGTGCTATTGCAAAGGCAATTGGATTTCCATGGCTCATGACAA | | |
| | TTATTGGGATAATTGATATTCTTTTTCCCCCTCTCTGCTTTTTTCTTCGAAGTCCACC | | |
| | TGCCAAAGAAGAAAAAATGGCTATTCTCATGGATCACAACTGCCCTATTAAAACAAAA | | |
| | ATGTACACTCAGAATAATATCCAGTCATATCCGATAGGTGAAGATGAAGAATCTGAAA | | |
| | GTGACTGAGATGAGATCCTCAAAAATCATCAAAGTGTTTAATTGTATAAAACAGTGTT | | |
| | TCCAGTGACACAACTCATCCAGAACTGTCTTAGTCATACCATCCATCCCTGGTGAAAG | | |
| | AGTAAAACCAAAGGTTATTATTTCCTTTCCATGGTTATGGTCGATTGCCAACAGCCTT | | |
| | ATAAAGAAAAAGAAGCTTTTCTAGGGGTTTGTATAAATAGTGTTGAAACTTTATTTTA | | |
| | TGTATTTAATTTTATTAAATATCATACAATATATTTTGATGAAATAGGTATTGTGTAA | | |
| | ATCTATAAATATTTGAATCCAAACCAAATATAATTTCC | | |
| | ORF Start: ATG at 40 | | ORF Stop: TGA at 1630 |
| | SEQ ID NO:338 | 530 aa | MW at 57130.4 kD |
| NOV39b, | MALSELALVRWLQESRRSRKLILFIVFLALLLDNMLLTVVGSSDPPFSASKGAGITVP | | |
| CG158583-02 | | | |
| Protein Sequence | IIPSYLYSIKHEKNATEIQTARPVHTASISDSFQSIFSYYDNSTMVTGNATRDLTLHQ | | |
| | TATQHMVTNASAVPSDCPSEDKDLLNENVQVGLLFASKATVQLITNPFIGLLTNRIGY | | |
| | PIPIFAGFCIHVVSTIMFAFSSSYAFLLIARSLQGIGSSCSSVAGMGMLASVYTDDEE | | |
| | RGNVMGIALGGLAMGVLVGPPFGSVLYEFVGKTAPFLVLAALVLLDGAIQLFVLQPSR | | |
| | VQPESQKGTPLTTLLKDPYILIAAGSICFANMGIAMLEPALPIWMMETMCSRKWQLGV | | |
| | AFLPASISYLIGTNIFGILAHKMGRWLCALLGMIIVGVSILCIPFAKNIYGLIAPNFG | | |
| | VGFAIGMVDSSMMPIMGYLVDLRHVSVYGSVYAIADVAFCMGYAIGPSAGGAIAKAIG | | |
| | FPWLMTIIGIIDILFAPLCFFLRSPPAKEEKMAILMDHNCPIKTKMYTQNNIQSYPIG | | |
| | EDEESESD | | |
| | SEQ ID NO:339 | 1647 bp | |
| NOV39c, | GGAGCCATGGCCCTGAGCGAGCTGGCGCTGGTCCGCTGGCTGCAGGACAGCCGCCGCT | | |
| CG158583-04 | | | |
| DNA Sequence | CGCGGAAGCTCATCCTGTTCATCGTGTTCCTGGCGCTGCTGCTGGACAACATGCTGCT | | |
| | CACTGTCGTGGTAGAGAGAGGGTTTCTCCATGTTGGCCAGCCTGGTCTCGAACTCCTG | | |
| | ACCTCAGGTGATCCACCTGCCTCAGCTTCCCAAAGTCCTGGAATTACAGTCCCCATCA | | |
| | TCCCAAGTTATCTGTACAGCATTAAGCATGAGAAGAATGCTACAGAAATCCAGACGGC | | |
| | CAGGCCAGTGCACACTGCCTCCATCTCAGACAGCTTCCAGAGCATCTTCTCCTATTAT | | |
| | GATAACTCGACTATGGTCACCGGGAATGCTACCAGAGACCTGACACTTCATCAGACCG | | |
| | CCACACAGCACATGGTGACCAACGCGTCCGCTGTTCCTTCCGACTGTCCCAGTGAAGA | | |
| | CAAAGACCTCCTGAATGAAAACGTGCAAGTTGGTCTGTTGTTTGCCTCGAAAGCCACC | | |
| | GTCCAGCTCATCACCAACCCTTTCATAGGACTACTGACCAACAGAATTGCCTATCCAA | | |
| | TTCCCATATTTGCGGGATTCTGCATCATGTTTGTCTCAACAATTATGTTTGCCTTCTC | | |
| | CAGCAGCTATGCCTTCCTGCTGATTGCCAGGTCGCTGCAGGGCATCGGCTCGTCCTGC | | |
| | TCCTCTGTGGCTGGGATGGGCATGCTTGCCAGTGTCTACACAGATGATGAAGAGAGAG | | |
| | GCAACGTCATGGGAATCGCCTTGGGAGGCCTGGCCATGGGGGTCTTAGTGGGCCCCCC | | |
| | CTTCGGGAGTGTGCTCTATGAGTTTGTGGGGAAGACGGCTCCGTTCCTGGTGCTGGCC | | |
| | GCCCTGGTACTCTTGGATGGAGCTATTCAGCTCTTTGTGCTCCAGCCGTCCCGGGTGC | | |
| | AGCCAGAGAGTCAGAAGGGGACACCCCTAACCACGCTGCTGAAGGACCCGTACATCCT | | |
| | CATTCCTGCAGGCTCCATCTGCTTTGCAAACATGGGCATCGCCATGCTGGAGCCAGCC | | |
| | CTGCCCATCTGGATGATCGAGACCATGTGTTCCCGAAAGTGGCAGCTGGGCGTTGCCT | | |
| | TCTTGCCAGCTAGTATCTCTTATCTCATTGGAACCAATATTTTTGGGATACTTGCACA | | |
| | CAAAATGGGGAGGTGGCTTTGTGCTCTTCTGGGAATGATAATTGTTGGAGTCAGCACT | | |
| | TTATGTATTCCATTTGCAAAAAACATTTATGGACTCATACCTCCGAACTTTGGAGTTG | | |
| | GTTTTGCAATTGGAATGGTGGATTCGTCAATGATGCCTATCATGGGCTACCTCGTAGA | | |
| | CCTGCGGCACGTGTCCGTCTATGGGAGTGTGTACGCCATTGCGGATGTGGCATTTTGT | | |
| | ATGGGGTATGCTATAGGTCCTTCTGCTGGTGGTGCTATTGCAAAGGCAATTGGATTTC | | |
| | CATGGCTCATGACAATTATTGGGATAATTGATATTCTTTTTGCCCCTCTCTGCTTTTT | | |
| | TCTTCGAAGTCCACCTACCAAAGAAGAAAAAATGGCTATTCTCATGGATCACAACTGC | | |
| | CCTATTAAAACAAAAATGTACACTCAGAATAGTATCCAGTCATATCCGATAGGTGAAG | | |
| | ATGAAGAATCTGAAAGTGACTGA | | |
| | ORF Start: ATG at 7 | | ORF Stop: TGA at 1645 |
| | SEQ ID NO:340 | 546 aa | MW at 58903.4 kD |
| NOV39c, | MALSELALVRWLQESRRSRKLILFIVFLALLLDNMLLTVVVERGFLHVGQPGLELLTS | | |
| CG158583-04 | | | |
| Protein Sequence | GDPPASASQSPGITVPIIPSYLYSIKHEKNATEIQTARPVHTASISDSPQSIFSYYDN | | |
| | STMVTGNATRDLTLHQTATQHMVTNASAVPSDCPSEDKDLLNENVQVGLLFASKATVQ | | |
| | LITNPFIGLLTNRIGYPIPIFAGFCIMFVSTIMFAFSSSYAFLLIARSLQGIGSSCSS | | |
| | VAGMGMLASVYTDDEERGNVMGIALGGLAMGVLVGPPFGSVLYEFVGKTAPFLVLAAL | | |
| | VLLDGAIQLFVLQPSRVQPESQKGTPLTTLLKDPYILIAAGSICFANMGIAMLEPALP | | |
| | IWMMETMCSRKWQLGVAFLPASISYLIGTNIFGILAHKMGRWLCALLGMITVGVSTLC | | |
| | IPFAKNIYGLIAPNFGVGFAIGMVDSSMMPIMGYLVDLRHVSVYGSVYAIADVAFCMG | | |
| | YAIGPSAGGAIAKAIGFPWLMTIIGIIDILFAPLCFFLRSPPTKEEKMAILMDHNCPI | | |
| | KTKMYTQNSIQSYPIGEDEESESD | | |
| | SEQ ID NO:341 | 1666 bp | |
| NOV39d, | GCAGGCATCGCAAGCGACCCCGAGCGGAGCCCCGGAGCCATGGCCCTGAGCGACCTGG | | |
| CG158583-05 | | | |
| DNA Sequence | CGCTGGTCCGCTGGCTGCAGGAGAGCCGCCGCTCGCGGAAGCTCATCCTGTTCATCGT | | |
| | GTTCCTGGCGCTGCTGCTGGACAACATGCTGCTCACTGTCGTGGGTTCAAGCGATCCT | | |
| | CCTTTCTCAGCCTCCAAAGGAGCTGGGATTACAGTCCCCATCATCCCAAGTTATCTGT | | |
| | ACAGCATTAAGCATGAGAAGAATGCTACAGAAATCCAGACGGCCAGGCCAGTGCACAC | | |
| | TGCCTCCATCTCAGACAGCTTCCAGGGCATCTTCTCCTATTATGATAACTCGACTATG | | |
| | GTCACCGGGAATGCTACCAGAGACCTGACACTTCATCAGACCGCCACACAGCACATGG | | |
| | TGACCAACGCGTCCGCTGTTCCTTCCGACTGTCCCAGTGAAGACAAAGACCTCCTGAA | | |
| | TGAAAACGTGCAAGTTGGTCTGTTGTTTGCCTCGAAAGCCACCGTCCAGCTCATCACC | | |
| | AACCCTTTCATAGGACTACTGACCAACAGAATTGGCTATCCAATTCCCATATTTGCGG | | |
| | GATTCTGCATCATGTTTGTCTCAACAATTATGTTTGCCTTCTCCAGCAGCTATGCCTT | | |
| | CCTGCTGATTGCCAGGTCGCTGCAGGGCATCGGCTCGTCCTGCTCCTCTGTGGCTGGG | | |
| | ATGGGCATGCTTGCCAGTGTCTACACAGATGATGAAGAGAGAGGCAACGTCATGGGAA | | |
| | TCGCCTTGGGAGGCCTGGCCATGGGGGTCTTAGTGGGCCCCCCCTTCGGGAGTGTGCT | | |
| | CTATGAGTTTGTGGGGAAGACGGCTCCGTTCCTGGTGCTGGCTGCCCTGGTACTCTTG | | |
| | GATGGAGCTATTCAGCTCTTTCTGCTCCAGCCGTCCCGGGTGCAGCCAGAGAGTCAGA | | |
| | AGGGGACACCCCTAACCACGCTGCTGAAGGACCCGTACATCCTCATTGCTGCAGGCTC | | |
| | CATCTGCTTTGCAAACATGGGCATCGCCATGCTGCAGCCAGCCCTGCCCATCTGGATG | | |
| | ATGGAGACCATGTGTTCCCGAAAGTGGCAGCTGGGCGTTGCCTTCTTGCCAGCTAGTA | | |
| | TCTCTTATCTCATTGGAACCAATATTTTTGGGATACTTGCACACAAAATGGGGAGGTG | | |
| | GCTTTGTGCTCTTCTGGGAATGATAATTGTTGGAGTCAGCATTTTATGTATTCCATTT | | |
| | GCAAAAAACATTTATGGACTCATAGCTCCGAACTTTGGAGTTGGTTTTGCAATTGGAA | | |
| | TGGTGGATTCGTCAATGATGCCTATCATGGGCTACCTCGTAGACCTGCGGCACGTGTC | | |
| | CGTCTATGGGAGTGTGTACGCCATTGCGGATGTGGCATTTTGTATGGGGTATGCTATA | | |
| | GGTCCTTCTGCTGGTGGTGCTATTGCAAAGGCAATTGGATTTCCATGGCTCATGACAA | | |
| | TTATTGGGATAATTGATATTCTTTTTGCCCCTCTCTGCTTTTTTCTTCGAAGTCCACC | | |
| | TGCCAAAGAAGAAAAAATGGCTATTCTCATGGATCACAACTGCCCTATTAAAACAAAA | | |
| | ATGTACACTCAGAATAATATCCAGTCATATCCGATAGGTGAAGATGAAGAATCTGAAA | | |
| | GTGACTGAGATGAGATCCTCAAAAATCATCAAAGTGTAAGGG | | |
| | ORF Start: ATG at 40 | | ORF Stop: TGA at 1630 |
| | SEQ ID NO:342 | 530 aa | MW at 57142.5 kD |
| NOV39d, | MALSELALVRWLQESRRSRKLILFIVFLALLLDNMLLTVVGSSDPPFSASKGAGITVP | | |
| CG158583-05 | | | |
| Protein Sequence | IIPSYLYSIKHEKNATEIQTARPVHTASISDSFQGIFSYYDNSTMVTGNATRDLTLHQ | | |
| | TATQHMVTNASAVPSDCPSEDKDLLNENVQVGLLFASKATVQLITNPFIGLLTNRIGY | | |
| | PIPIFAGFCIMFVSTIMFAFSSSYAFLLIARSLQGIGSSCSSVAGMGMLASVYTDDEE | | |
| | RGNVMGIALGGLAMGVLVGPPFGSVLYEFVGKTAPFLVLAALVLLDGAIQLFVLQPSR | | |
| | VQPESQKGTPLTTLLKDPYILIAAGSICFANMGIANLEPALPIWMMETMCSRKWQLGV | | |
| | AFLPASISYLIGTNTFGILAHKMGRWLCALLGMIIVGVSILCIPFAKNIYGLIAPNFG | | |
| | VGFAIGMVDSSMNPIMGYLVDLRHVSVYGSVYAIADVAFCMGYAIGPSAGGAIAKAIG | | |
| | FPWLMTIIGIIDILFAPLCFFLRSPPAKEEKMAILMDHNCPIKTKMYTQNNIQSYPIG | | |
| | EDEESESD | | |
| | SEQ ID NO:343 | 1618 bp | |
| NOV39e, | GCAGGCATCGCAAGCGACCCCGAGCGGAGCCCCGGAGCCATGGCCCTGAGCGAGCTGG | | |
| CG158583.03 | | | |
| DNA Sequence | CGCTGGTCCGCTGGCTGCAGGAGAGCCGCCGCTCGCGGAAGCTCATCCTGTTCATCGT | | |
| | GTTCCTGGCGCTGCTGCTGGACAACATGCTGCTCACTGTCGTGGTCCCCATCATCCCA | | |
| | AGTTATCTGTACAGCATTAAGCATGAGAAGAATGCTACAGAAATCCAGACGGCCAGGC | | |
| | CAGTGCACACTGCCTCCATCTCAGACAGCTTCCAGAGCATCTTCTCCTATTATGATAA | | |
| | CTCGACTATGGTCACCGGGAATGCTACCAGAGACCTGACACTTCATCAGACCGCCACA | | |
| | CAGCACATGGTGACCAACGCGTCCGCTGTTCCTTCCGACTGTCCCAGTGAAGACAAAG | | |
| | ACCTCCTGAATGAAAACGTGCAAGTTGGTCTGTTGTTTGCCTCGAAAGCCACCGTCCA | | |
| | GCTCATCACCAACCCTTTCATAGGACTACTGACCAACAGAATTGGCTATCCAATTCCC | | |
| | ATATTTGCGGGATTCTGCATCATGTTTGTCTCAACAATTATGTTTGCCTTCTCCAGCA | | |
| | GCTATGCCTTCCTGCTGATTGCCAGGTCGCTGCAGGGCATCGGCTCGTCCTGCTCCTC | | |
| | TGTGGCTGGGATGGGCATGCTTGCCAGTGTCTACACAGATGATGAAGAGAGAGGCAAC | | |
| | GTCATGGGAATCGCCTTGGGAGGCCTGGCCATGGGGGTCTTAGTGGGCCCCCCCTTCG | | |
| | GGAGTGTGCTCTATGAGTTTGTGGGGAAGACGGCTCCGTTCCTGGTGCTGGCCGCCCT | | |
| | GGTACTCTTGGATGGAGCTATTCAGCTCTTTGTGCTCCAGCCGTCCCGGGTGCAGCCA | | |
| | GAGAGTCAGAAGGGGACACCCCTAACCACGCTGCTGAAGGACCCGTACATCCTCATTG | | |
| | CTGCAGGCTCCATCTGCTTTGCAAACATGGGCATCGCCATGCTGGAGCCAGCCCTGCC | | |
| | CATCTGGATGATGGAGACCATGTGTTCCCGAAAGTGGCAGCTGGGCGTTGCCTTCTTG | | |
| | CCAGCTAGTATCTCTTATCTCATTGGAACCAATATTTTTGGGATACTTGCACACAAAA | | |
| | TGGGGAGGTGGCTTTGTGCTCTTCTGGGAATGATAATTGTTGGAGTCAGCACTTTATG | | |
| | TATTCCATTTGCAAAAAACATTTATGGACTCATAGCTCCGAACTTTGGAGTTGGTTTT | | |
| | GCAATTGGAATGGTGGATTCGTCAATGATGCCTATCATGGGCTACCTCGTAGACCTGC | | |
| | GGCACGTGTCCGTCTATGGGAGTGTGTACGCCATTGCGGATGTGGCATTTTGTATGGG | | |
| | GTATGCTATAGGTCCTTCTGCTGGTGGTGCTATTGCAAAGGCAATTGGATTTCCATGG | | |
| | CTCATGACAATTATTGGGATAATTGATATTCTTTTTGCCCCTCTCTGCTTTTTTCTTC | | |
| | GAAGTCCACCTGCCAAAGAAGAAAAAATGGCTATTCTCATGGATCACAACTGCCCTAT | | |
| | TAAAACAAAAATGTACACTCAGAATAGTATCCAGTCATATCCGATAGGTGAAGATGAA | | |
| | GAATCTGAAAGTGACTGAGATGAGATCCTCAAAAATCATCAAAGTGTAAGGG | | |
| | ORF Start: ATG at 40 | | ORF Stop: TGA at 1582 |
| | SEQ ID NO:344 | 514 aa | MW at 55672.9 kD |

| | |
|---|---|
| NOV39e, | MALSELALVRWLQESRRSRKLILFIVFLALLLDNMLLTVVVPIIPSYLYSIKHEKNAT |
| CG158583-03 | |
| Protein Sequence | EIQTARPVHTASISDSFQSIFSYYDNSTMVTGNATRDLTLHQTATQHMVTNASAVPSD |
| | CPSEDKDLLNENVQVGLLFASKATVQLITNPFIGLLTNRIGYPIPIFAGFCIMFVSTI |
| | MFAFSSSYAFLLIARSLQGIGSSCSSVAGMGMLASVYTDDEERGNVMGIALGGLAMGV |
| | LVGPPFGSVLYEFVGKTAPFLVLAALVLLDGAIQLFVLQPSRVQPESQKGTPLTTLLK |
| | DPYILIAAGSICFANMGIAMLEPALPIWMMETMCSRKWQLGVAFLPASISYLIGTNIF |
| | GILAHKMGRWLCALLGMIIVGVSTLCIPFAKNIYGLIAPNFGVGFAIGMVDSSMMPIM |
| | GYLVDLRHVSVYGSVYAIADVAFCMGYAIGPSAGGAIAKAIGFPWLMTIIGIIDILFA |
| | PLCFFLRSPPAKEEKMAILMDHNCPIKTKMYTQNSIQSYPIGEDEESESD |

Sequence comparison of the above protein sequences yields the following sequence relationships shown in Table 39B.

TABLE 39B
Comparison of NOV39a against NOV39b through NOV39e.

| | | Identities/ |
|---|---|---|
| | | Similarities for |
| Protein | NOV39a Residues/ | the Matched |
| Sequence | Match Residues | Region |
| NOV39b | 1 . . . 546 | 522/546 (95%) |
| | 1 . . . 530 | 523/546 (95%) |
| NOV39c | 1 . . . 546 | 543/546 (99%) |
| | 1 . . . 546 | 544/546 (99%) |
| NOV39d | 1 . . . 546 | 523/546 (95%) |
| | 1 . . . 530 | 524/546 (95%) |
| NOV39e | 1 . . . 546 | 512/546 (93%) |
| | 1 . . . 514 | 513/546 (93%) |

Further analysis of the NOV39a protein yielded the following properties shown in Table 39C.

TABLE 39C
Protein Sequence Properties NOV39a

| | |
|---|---|
| PSort | 0.6400 probability located in plasma membrane; 0.4600 |
| analysis: | probability located in Golgi body; 0.3700 probability |
| | located in endoplasmic reticulum (membrane); 0.1000 |
| | probability located in endoplasmic reticulum (lumen) |
| SignalP | Cleavage site between residues 38 and 39 |
| analysis: | |

A search of the NOV39a protein against the Geneseq database, a proprietary database that contains sequences published in patents and patent publication, yielded several homologous proteins shown in Table 39D.

TABLE 39D
Geneseq Results for NOV39a

| | | | Identities/ | |
|---|---|---|---|---|
| | | | Similarities for | |
| Geneseq | Protein/Organism/Length | NOV39a Residues/ | the Matched | Expect |
| Identifier | [Patent #, Date] | Match Residues | Region | Value |
| ABB09288 | Human solute carrier family 18 | 1 . . . 546 | 514/546 (94%) | 0.0 |
| | member 2 (SLC18A2) protein SEQ | 1 . . . 514 | 514/546 (94%) | |
| | ID NO: 3 - Homo sapiens, 514 aa. | | | |
| | [WO200222652-A2, 21 MAR. | | | |
| | 2002] | | | |
| AAW38286 | Human synaptic vesicle amine | 1 . . . 546 | 514/546 (94%) | 0.0 |
| | transporter protein - Homo sapiens, | 1 . . . 514 | 514/546 (94%) | |
| | 514 aa. [US5688936-A, 18 NOV. | | | |
| | 1997] | | | |
| AAR47342 | Mammalian synaptic vesicle amine | 1 . . . 546 | 514/546 (94%) | 0.0 |
| | transporter protein - Homo sapiens, | 1 . . . 514 | 514/546 (94%) | |
| | 514 aa. [WO9325699-A, 23 DEC. | | | |
| | 1993] | | | |
| AAW38285 | Rat synaptic vesicle amine | 1 . . . 546 | 470/551 (85%) | 0.0 |
| | transporter protein - Rattus rattus, | 1 . . . 515 | 490/551 (88%) | |
| | 515 aa. [US5688936-A, 18 NOV. | | | |
| | 1997] | | | |
| AAR47335 | Mammalian synaptic vesicle amine | 1 . . . 546 | 470/551 (85%) | 0.0 |
| | transporter protein - Rattus rattus, | 1 . . . 515 | 490/551 (88%) | |
| | 515 aa. [WO9325699-A, 23 DEC. | | | |
| | 1993] | | | |

In a BLAST search of public sequence datbases, the NOV39a protein was found to have homology to the proteins shown in the BLASTP data in Table 39E.

TABLE 39E
Public BLASTP Results for NOV39a

| | | | Identities/ | |
|---|---|---|---|---|
| Protein | | | Similarities for | |
| Accession | | NOV39a Residues/ | the Matched | Expect |
| Number | Protein/Organism/Length | Match Residues | Portion | Value |
| Q05940 | Synaptic vesicle amine transporter | 1 . . . 546 | 514/546 (94%) | 0.0 |
| | (Monoamine transporter) (Vesicular | 1 . . . 514 | 514/546 (94%) | |
| | amine transporter 2) (VAT2) - Homo | | | |
| | sapiens (Human), 514 aa. | | | |
| Q9H3P6 | Synaptic vesicle monoamine | 4 . . . 546 | 511/543 (94%) | 0.0 |
| | transporter - Homo sapiens | 12 . . . 522 | 511/543 (94%) | |
| | (Human), 522 aa. | | | |
| S29810 | monoamine transport protein - | 1 . . . 546 | 510/546 (93%) | 0.0 |
| | human, 514 aa. | 1 . . . 514 | 510/546 (93%) | |
| Q27963 | Synaptic vesicle amine transporter | 1 . . . 546 | 471/549 (85%) | 0.0 |
| | (Monoamine transporter) (Vesicular | 1 . . . 517 | 492/549 (88%) | |
| | amine transporter 2) (VAT2) - Bos | | | |
| | taurus (Bovine), 517 aa. | | | |
| A46374 | resernine-sensitive vesicular | 1 . . . 546 | 472/551 (85%) | 0.0 |
| | monoamine transporter - rat, 515 aa. | 1 . . . 515 | 492/551 (88%) | |

PFam analysis predicts that the NOV39a protein contains the domains shown in the Table 39F.

TABLE 39F
Domain Analysis of NOV39a

| | | Identities/ | |
|---|---|---|---|
| | | Similarities for | |
| Pfam | NOV39a | the Matched | Expect |
| Domain | Match Region | Region | Value |
| sugar_tr | 98 . . . 516 | 66/523 (13%) | 0.019 |
| | | 268/523 (51%) | |

### Example 40

The NOV40 clone was analyzed, and the nucleotide and encoded polypeptide sequences are shown in Table 40A.

TABLE 40A
NOV40 Sequence Analysis

| | |
|---|---|
| | SEQ ID NO: 345 1096 bp |
| NOV40a, GCAACCGGGGCAGGCCGTGCCGGCTGAGGAGGTCCTGAGGCTACAGAGCTGCCGCGGC | |
| CG158964-01 | |
| DNA Sequence | TGGCACACGAGCGCCTCGGCACTAACCGAGTGTTCGCCGGGGCTGTGAGGGGAGGGCC |
| | CCGGGCGCCATTGCTGGCGGTGGGAGCGCCGCCCCGTCTCAGCCCGCCCTCGGCTGCT |
| | CTCCTCCTCCGGCTGGGAGGGGCCGTAGCTCGGGGCCGTCGCCAGCCCCGGCCCGGGC |
| | TCGAGAATCAAGGGCCTCGGCCGCCGTCCCGCAGCTCAGTCCATCGCCCTTGCCGGGC |
| | AGCCCGGGCAGAGACCATGTTTGACAAGACGCGGCTGCCGTACGTGGCCCTCGATGTG |
| | CTCTGCGTGTTGCTGGATTATTCTTGGAGAAACCCTGTCTGTTTACTGTAACCTTTTG |
| | CACTCAAATTCCTTTATCAGGAATAACTACATAGCCACTATTTACAAAGCCATTGGAA |
| | CCTTTTTATTTGGTGCAGCTGCTAGTCAGTCCCTGACTGACATTGCCAAGTATTCAAT |
| | AGGCAGACTGCGGCCTCACTTCTTGGATGTTTGTGATCCAGATTGGTCAAAAATCAAC |
| | TGCAGCGATGGTTACATTGAATACTACATATGTCGAGGGAATGCAGAAAGAGTTAAGG |
| | AAGGCAGGTTGTCCTTCTATTCAGGCCACTCTTCGTTTTCCATGTACTGCATGCTGTT |
| | TGTGGCACTTTATCTTCAAGCCAGGATGAAGGGAGACTGGGCAAGACTCTTACGCCCC |
| | ACACTGCAATTTGGTCTTGTTGCCGTATCCATTTATGTGGGCCTTTCTCGAGTTTCTG |
| | ATTATAAACACCACTGGAGCGATGTGTTGACTGGACTCATTCAGGGAGCTCTGGTTGC |
| | AATATTAGTTGCTGTATATGTATCGGATTTCTTCAAAGAAAGAACTTCTTTTAAAGAA |
| | AGAAAAGAGGAGGACTCTCATACAACTCTGCATGAAACACCAACAACTGGGAATCACT |
| | ATCCGAGCAATCACCAGCCTTGAAAGGCAGCAGGGTGCCCAGGTGAAGCTGGCCTGTT |
| | TTCTAAAGGAAAATGATTGCCACAAGGCAAGAGGATGCATCTTTCTTCCTGG |
| | ORF Start: ATG at 344 ORF Stop: TGA at 1007 |
| | SEQ ID NO: 346 221 aa MW at 25083.4kD |
| NOV40a, | MCSACCWIILGETLSVYCNLLHSNSFIRNNYIATIYKAIGTFLFGAAASQSLTDIAKY |
| CG158964-01 | |
| Protein Sequence | SIGRLRPHFLDVCDPDWSKINCSDGYIEYYICRGNAERVKEGRLSFYSGHSSFSMYCM |
| | LFVALYLQARMKGDWARLLRPTLQFGLVAVSIYVGLSRVSDYKHHWSDVLTGLIQGAL |
| | VAILVAVYVSDFFKERTSFKERKEEDSHTTLHETPTTGNHYPSNHQP |
| | SEQ ID NO: 347 1388 bp |
| NOV40b, | CGGCCGCGTCGACGCAACCGGGGCAGGCCGTGCCGGCTGAGGAGGTCCTGAGGCTACA |
| CG158964-02 | |
| DNA Sequence | GAGCTGCCGCGGCTGGCACACGAGCGCCTCGGCACTAACCGAGTGTTCGCGGGGGCTG |
| | TGAGGGGAGGGCCCCGGGCGCCATTGCTGGCGGTGGGAGCGCCGCCCGGTCTCAGCCC |
| | GCCCTCGGCTGCTCTCCTCCTCCGGCTGGGAGGGGCCGTAGCTCGGGGCCGTCGCCAG |
| | CCCCGGCCCGGGCTCGAGAATCAAGGGCCTCGGCCGCCGTCCCGCAGCTCAGTCCATC |
| | GCCCTTGCCGGGCAGCCCGGGCAGAGACCATGTTTGACAAGACGCGGCTGCCGTACGT |
| | GGCCCTCGATGTGCTCTGCGTGTTGCTGGATTATTCTTGGAGAAACCCTGTCTGTTTA |
| | CTGTAACCTTTTGCACTCAAATTCCTTTATCAGGAATAACTACATAGCCACTATTTAC |
| | AAAGCCATTGGAACCTTTTTATTTGGTGCAGCTGCTAGTCAGTCCCTGACTGACATTG |
| | CCAAGTATTCAATAGGCAGACTCCGGCCTCACTTCTTGGATGTTTGTGATCCAGATTG |
| | GTCAAAAATCAACTGCAGCGATGGTTACATTGAATACTACATATGTCGAGGGAATGCA |
| | GAAAGAGTTAAGGAAGGCAGGTTGTCCTTCTATTCAGGCCACTCTTCGTTTTCCATGT |
| | ACTGCATGCTGTTTGTGGCACTTTATCTTCAAGCCAGGATGAAGGGAGACTGGGCAAG |
| | ACTCTTACGCCCCACACTGCAATTTGGTCTTGTTGCCGTATCCATTTATGTGGGCCTT |
| | TCTCGAGTTTCTGATTATAAACACCACTGGAGCGATGTGTTGACTGGACTCATTCAGG |
| | GAGCTCTGGTTGCAATATTAGTTGCTGTATATGTATCGGATTTCTTCAAAGAAAGAAC |
| | TTCTTTTAAAGAAAGAAAAGAGGAGGACTCTCATACAACTCTGCATGAAACACCAACA |
| | ACTGGGAATCACTATCCGAGCAATCACCAGCCTTGAAAGGCAGCAGGGTGCCCAGGTG |
| | AAGCTGGCCTGTTTTCTAAAGGAAAATGATTGCCACAAGGCAAGAGGATGCATCTTTC |
| | TTCCTGGTGTACAAGCCTTTAAAGACTTCTGCTGCTGCTATGCCTCTTGGATGCACAC |
| | TTTGTGTGTACATAGTTACCTTTAACTCAGTGGTTATCTAATAGCTCTAAACTCATTA |
| | AAAAAACTCCAAGCCTTCCACCAAAACAGTGCCCCACCTGTATACATTTTTATTAAAA |
| | AAATGTAATGCTTATGTATAAACATGTATGTAATATGCTTTCTATGAATGATGTTTGA |
| | TTTAAATATAATACATATTAAAATGTATGGGAGAACCAAAAAAAAAAAAAAAAA |
| | ORF Start: ATG at 357 ORR Stop: TGA at 1020 |
| | SEQ ID NO: 348 221 aa MW at 25083.4kD |
| NOV40b, | MCSACCWIILGETLSVYCNLLHSNSFIRNNYIATIYKAIGTFLFGAAASQSLTDIAKY |
| CG158964-02 | |
| Protein Sequence | SIGRLRPHFLDVCDPDWSKTNCSDGYIEYYICRGNAERVKEGRLSFYSGHSSFSMYCM |
| | LFVALYLQARMKGDWARLLRPTLQFGLVAVSIYVGLSRVSDYKHHWSDVLTGLIQGAL |
| | VATLVAVYVSDFFKERTSFKERKEEDSHTTLHETPTTGNNYPSNHQP |

Sequence comparison of the above protein sequences yields the following sequence relationships shown in Table 40B.

TABLE 40B
Comparison of NOV40a against NOV40b.

| | | Identities/ |
|---|---|---|
| | | Similarities for |
| Protein | NOV40a Residues/ | the Matched |
| Sequence | Match Residues | Region |
| NOV40b | 1 . . . 221 | 221/221 (100%) |
| | 1 . . . 221 | 221/221 (100%) |

Further analysis of the NOV40a protein yielded the following properties shown in Table 40C.

TABLE 40C
Protein Sequence Properties NOV40a

| | |
|---|---|
| PSort | 0.6400 probability located in endoplasmic reticulum |
| analysis: | (membrane); 0.4960 probability located in plasma |
| | membrane; 0.3776 probability located in microbody |
| | (peroxisome); 0.1900 probability located in Golgi body |
| SignalP | Cleavage site between residues 49 and 50 |
| analysis: | |

A search of the NOV40a protein against the Geneseq database, a proprietary database that contains sequences published in patents and patent publication, yielded several homologous proteins shown in Table 40D.

TABLE 40D
Geneseq Results for NOV40a

| | | | Identities/ | |
|---|---|---|---|---|
| | | | Similarities for | |
| Geneseq | Protein/Organism/Length | NOV40a Residues/ | the Matched | Expect |
| Identifier | [Patent #, Date] | Match Residues | Region | Value |
| AAY24916 | Human phosphatase HPA-1 - | 8 . . . 221 | 214/214 (100%) | e−125 |
| | Homo sapiens, 285 aa. | 72 . . . 285 | 214/214 (100%) | |
| | [WO9931225-A2, 24 JUN. 1999] | | | |
| AAW79284 | Human phosphatidic acid | 8 . . . 221 | 214/214 (100%) | e−125 |
| | phosphatase alpha 1 - Homo | 71 . . . 284 | 214/214 (100%) | |
| | sapiens, 284 aa. [WO9846730-A1, | | | |
| | 22 OCT. 1998] | | | |
| AAW79285 | Human phosphatidic acid | 8 . . . 221 | 213/214 (99%) | e−124 |
| | phosphatase alpha 2 - Homo | 72 . . . 285 | 213/214 (99%) | |
| | sapiens, 285 aa. [WO9846730-A1, | | | |
| | 22 OCT. 1998] | | | |
| AAW79287 | Human phosphatidic acid | 11 . . . 200 | 123/190 (64%) | 2e−66 |
| | phosphatase gamma - Homo | 72 . . . 260 | 145/190 (75%) | |
| | sapiens, 276 aa. [WO9846730-A1, | | | |
| | 22 OCT. 1998] | | | |
| AAW79286 | Human phosphatidic acid | 8 . . . 192 | 113/185 (61%) | 5e−59 |
| | phosphatase beta - Homo sapiens, | 100 . . . 283 | 138/185 (74%) | |
| | 311 aa. [WO9846730-A1, 22 | | | |
| | OCT. 1998] | | | |

In a BLAST search of public sequence datbases, the NOV40a protein was found to have homology to the proteins shown in the BLASTP data in Table 40E.

TABLE 40E
Public BLASTP Results for NOV40a

| | | | Identities/ | |
|---|---|---|---|---|
| Protein | | | Similarities for | |
| Accession | | NOV40a Residues/ | the Matched | Expect |
| Number | Protein/Organism/Length | Match Residues | Portion | Value |
| O14494 | PHOSPHATIDIC acid | 8 . . . 221 | 214/214 (100%) | e−124 |
| | phosphatase 2A (EC 3.1.3.4) - | 71 . . . 284 | 214/214 (100%) | |
| | Homo sapiens (Human), 284 aa. | | | |
| O60463 | Type-2 phosphatidic acid | 8 . . . 221 | 214/214 (100%) | e−124 |
| | phosphohydrolase - Homo sapiens | 76 . . . 289 | 214/214 (100%) | |
| | (Human), 289 aa. | | | |
| O60457 | Type-2 phosphatidic acid | 8 . . . 221 | 213/214 (99%) | e−123 |
| | phosphatase alpha-2 (EC 3.1.3.4) - | 72 . . . 285 | 213/214 (99%) | |
| | Homo sapiens (Human), 285 aa. | | | |
| O88957 | Phosphatidic acid phosphatase 2a2 - | 8 . . . 221 | 199/215 (92%) | e−116 |
| | Cavia porcellus (Guinea pig), | 72 . . . 286 | 208/215 (96%) | |
| | 286 aa. | | | |
| O88956 | Phosphatidic acid phosphatase 2a - | 8 . . . 221 | 198/215 (92%) | e−116 |
| | Cavia porcellus (Guinea pig), 285 | 71 . . . 285 | 208/215 (96%) | |
| | aa. | | | |

PFam analysis predicts that the NOV40a protein contains the domains shown in the Table 40F.

TABLE 40F
Domain Analysis of NOV40a

| | | Identities/ | |
|---|---|---|---|
| | | Similarities for | |
| Pfam | NOV40a | the Matched | Expect |
| Domain | Match Region | Region | Value |
| PAP2 | 37 . . . 188 | 62/174 (36%) | 1.5e−50 |
| | | 133/174 (76%) | |

### Example 41

The NOV41 clone was analyzed, and the nucleotide and encoded polypeptide sequences are shown in Table 41A.

TABLE 41A
NOV41 Sequence Analysis

| | SEQ ID NO:349 | 1524 bp | |
|---|---|---|---|
| NOV41a, | AACCAGATGGGAAAAATGTCTATCTGTACTTTTCAATCAACTGAGAAAGACGAGAAAA | | |
| CG15908-01 | | | |
| DNA Sequence | AAGAAGCTCACACTGGGAATGTAATGACTAATTTATCAAATGCTTATGCTTCAGATTT | | |
| | GTTGCTATGCAAAGATGACAAAGACTTAACAAAATATTTTCTGCTGCAAGTGGTAGAT | | |
| | ATTCTTCTACAGTATGCAAAAAACACCTTTGATGGTAAGAGTGAAATATTGGACTTCC | | |
| | ATCATCCTCATCAACTACTTGAAGGTTTGGTTGGGTTTACCTTAGAACTGCCTGACCA | | |
| | CCCTGAATCTCTGGAACAGTTACTTGCTGATTGCACAGATACCTTAAAATACAGTGTT | | |
| | AAAACAGGTCATCCTCGCTATTTTAACCAGCTGTCCAGTGGGTTAGATATGACTGGAC | | |
| | TTGCAGGGGAATGGTTGACAGCCACTGCAAATACCAACCTGTTTACATATGAAATAGC | | |
| | CCCAGTTTTTACTGTCATGGAGACAATTCTTCTCAAGAAAATGTATGAAATTATTGGC | | |
| | TGGGGGAAGAAACAAGCAGATGGAATATTTTCACCTGGTGGCAGTATATCAAGCCTTT | | |
| | ATGGTATTTTAGTAGCTCACTATAAACAATATCCAGAGATAAAAACAAAAGGCATGAC | | |
| | TGCACTTCCATGCATTGTATTATTTGTTTCTGAGCAAGGTCATTACTCAATAAAAATA | | |
| | GCTGCAACAATTTTGGGTATTGGAATTGATAATGTAATTGAAGTAAAGTGTGATGAAA | | |
| | GGGGAAAGATGATTCCAGCTGAGTTAGAGAAAAATATATTACAAGCTAAAAAAAAAGG | | |
| | TCAAACTCCATTCTGTGTCTGTGCCACAGCCGGAAGCACAGTGTACGGAGCCTTCGAC | | |
| | CCTCTCCCTGACATCGCTGATATTTGTGAGAAGCACAAACTCTGGATGCATGTGGATG | | |
| | CAGCTTCGGGAGGTGGACTGCTGCTATCCAGAAACTATTCCTATAAACTCAGTGGTAT | | |
| | TGAAAGGGCCAAGTCTGTGACCTGGAATCCACACAAACTAATGGGTGTCCCTCTTCAG | | |
| | TGCTCTGCTATCTTGATCCGGGAAAAAGGCCTTCTAGATGCATGTAATCAGATGCAAG | | |
| | CTGAATATCTTTTCCAGTCAGGTAAACTCTACAATGTTGACTTTGACACGGCGGATAA | | |
| | AACTATTCAGTGTGGCCGACATGTTGATATCTTCAAGCAGTGGTTAATGTGGAAAGCA | | |
| | AAGGGAACCCTTGGCTTTGAGGAACAAATCAACAAATATATGGAACTTGCAAAATACT | | |
| | TCTATAAGGTTTTAAAGAAAAAAGATAACTTTAAGCTTGTGTTTGATGCAGAGCCTGA | | |
| | GTTCACTAATGTCTGCTTCTGGTATTTCCCAGCAAGGCTTAAACATATTCCAAAAGGT | | |
| | TTTGAAAGAGATCAAGAACTCCGAAAGGTAGCTCCAAAGATTAAAGCACAGATGATGA | | |
| | TGGAAGGCACAATCATGATAAGCTACCAGCCATGTGGAGACAAAGTAAATATTTTGCG | | |
| | AATGGTTTTTTTCTAA | | |
| | ORF Start: ATG at 7 | | ORF Stop: TAA at 1522 |
| | SEQ ID NO:350 | 505 aa | MW at 57169.9 kD |

| | |
|---|---|
| NOV41a, | MGKMSICTFQSTEKDEKKEAHTGNVMTNLSNAYASDLLLCKDDKDLTKYFLLQVVDIL |
| CG159084-01 | |
| Protein Sequence | LQYAKNTFDGKSEILDFHHPHQLLEGLVGFTLELPDHPESLEQLLADCTDTLKYSVKT |
| | GHPRYFNQLSSGLDMTGLAGEWLTATANTNLFTYEIAPVFTVMETILLKKMYEIIGWG |
| | KKQADGIFSPGGSISSLYGILVAHYKQYPEIKTKGMTALPCIVLFVSEQGHYSIKIAA |
| | TILGIGIDNVIEVKCDERGKMIPAELEKNILQAKKKGQTPFCVCATAGSTVYGAFDPL |
| | PDIADICEKHKLWMHVDAAWGGGLLLSRNYSYKLSGIERAKSVTWNPHKLMGVPLQCS |
| | AILIREKGLLDACNQMQAEYLFQSGKLYNVDFDTADKTIQCGRHVDIFKQWLMWKAKG |
| | TLGFEEQINKYMELAKYFYKVLKKKDNFKLVFDAEPEFTNVCFWYFPARLKHIPKGFE |
| | RDQELRKVAPKIKAQMMMEGTIMISYQPCGDKVNILRNVFF |

Further analysis of the NOV41a protein yielded the following properties shown in Table 41B.

TABLE 41B
Protein Sequence Properties NOV41a

| | |
|---|---|
| PSort | 0.5819 probability located in microbody (peroxisome); |
| analysis: | 0.1000 probability located in mitochondrial matrix space; |
| | 0.1000 probability located in lysosome (lumen); 0.0000 |
| | probability located in endoplasmic reticulum (membrane) |
| SignalP | No Known Signal Sequence Predicted |
| analysis: | |

A search of the NOV41a protein against the Geneseq database, a proprietary database that contains sequences published in patents and patent publication, yielded several homologous proteins shown in Table 41C.

TABLE 41C
Geneseq Results for NOV41a

| | | | Identities/ | |
|---|---|---|---|---|
| | | | Similarities for | |
| Geneseq | Protein/Organism/Length | NOV41a Residues/ | the Matched | Expect |
| Identifier | [Patent #, Date] | Match Residues | Region | Value |
| AAY57064 | Glutamate decarboxylase 67 (GAD- | 14 . . . 503 | 319/490 (65%) | 0.0 |
| | 67) amino acid sequence - Homo | 80 . . . 569 | 387/490 (78%) | |
| | sapiens, 594 aa. [WO9956763-A1, | | | |
| | 11 NOV. 1999] | | | |
| AAR27221 | Full length brain GAD - Homo | 14 . . . 503 | 319/490 (65%) | 0.0 |
| | sapiens, 594 aa. [WO9214485-A, 03 | 80 . . . 569 | 387/490 (78%) | |
| | SEP. 1992] | | | |
| AAR27220 | Brain GAD #2 - Mus musculus, 593 | 27 . . . 503 | 317/477 (66%) | 0.0 |
| | aa. [WO9214485-A, 03 SEP. 1992] | 92 . . . 568 | 378/477 (78%) | |
| AAB03072 | Chimeric human GAD67/rat GAD65 | 14 . . . 503 | 310/490 (63%) | 0.0 |
| | glutamic acid decarboxylase, SEQ | 80 . . . 569 | 388/490 (78%) | |
| | ID NO: 4 - Chimeric - Homo sapiens, | | | |
| | 594 aa. [US6060593-A, 09 MAY | | | |
| | 2000] | | | |
| AAY33656 | Chimeric rat GAD65/human GAD67 | 14 . . . 503 | 310/490 (63%) | 0.0 |
| | fusion protein 2 - Synthetic, 594 aa. | 80 . . . 569 | 388/490 (78%) | |
| | [US5968757-A, 19 OCT. 1999] | | | |

In a BLAST search of public sequence datbases, the NOV41a protein was found to have homology to the proteins shown in the BLASTP data in Table 41D.

TABLE 41D
Public BLASTP Results for NOV41a

| | | | Identities/ | |
|---|---|---|---|---|
| Protein | | | Similarities for | |
| Accession | | NOV41a Residues/ | the Matched | Expect |
| Number | Protein/Organism/Length | Match Residues | Portion | Value |
| Q9YI58 | Glutamate decarboxylase 67 - Gallus | 14 . . . 503 | 322/490 (65%) | 0.0 |
| | gallus (Chicken), 590 aa. | 76 . . . 565 | 388/490 (78%) | |
| B41935 | glutamate decarboxylase (EC | 14 . . . 503 | 319/490 (65%) | 0.0 |
| | 4.1.1.15) 1 - human, 594 aa. | 80 . . . 569 | 387/490 (78%) | |
| Q99259 | Glutamate decarboxylase, 67 kDa | 14 . . . 503 | 319/490 (65%) | 0.0 |
| | isoform (EC 4.1.1.15) (GAD-67) (67 | 80 . . . 569 | 387/490 (78%) | |
| | kDa glutamic acid decarboxylase) - | | | |
| | Homo sapiens (Human), 594 aa. | | | |
| S48135 | glutamate decarboxylase (EC | 14 . . . 503 | 318/490 (64%) | 0.0 |
| | 4.1.1.15) - human, 593 aa. | 79 . . . 568 | 387/490 (78%) | |
| S51776 | glutamate decarboxylase (EC | 14 . . . 503 | 318/490 (64%) | 0.0 |
| | 4.1.1.15) - human, 593 aa. | 79 . . . 568 | 387/490 (78%) | |

PFam analysis predicts that the NOV41a protein contains the domains shown in the Table 41E.

TABLE 41E
Domain Analysis of NOV41a

| | | Identities/ | |
|---|---|---|---|
| | | Similarities for | |
| Pfam | NOV41a | the Matched | Expect |
| Domain | Match Region | Region | Value |
| pyridoxal_deC | 78 . . . 452 | 136/401 (34%) | 6.9e−154 |
| | | 322/401 (80%) | |

### Example 42

The NOV42 clone was analyzed, and the nucleotide and encoded polypeptide sequences are shown in Table 42A.

TABLE 42A
NOV42 Sequence Analysis

| | SEQ ID NO:351 | 2990 bp | |
|---|---|---|---|
| NOV42a, | CCGGCGCCGGGCGGCCGGCGAGTCTGGAGCCCGCCCCGTCGCCGGCCGCGTCCTCCGG | | |
| CG159130-01 | | | |
| DNA Sequence | GCATGGAAGGAGGCGGCAAGCCCAACTCTTCGTCTAACAGCCGGGACGATGGCAACAG | | |
| | CGTCTTCCCCGCCAAGGCGTCCGCGCCGGGCGCGGGGCCGGCCGCGGCCGAGAAGCGC | | |
| | CTGGGCACCCCGCCGGGGGGCGGCGGGGCCGGCGCGAAGGAGCACGGCAACTCCGTGT | | |
| | GCTTCAAGGTGGACGGCGGTGGCGGCGGTGGCGGCGGCGGCGGCGGCGGCGAGGAGCC | | |
| | GGCGGGGGGCTTCGAAGACGCCGAGGGGCCCCGGCGGCAGTACGGCTTCATGCAGAGG | | |
| | CAGTTCACCTCCATGCTGCAGCCCGGGGTCAACAAATTCTCCCTCCGCATGTTTGGGA | | |
| | GCCAGAAGGCGGTGGAAAAGGAGCAGGAAAGGGTTAAAACTGCAGGCTTCTGGATTAT | | |
| | CCACCCTTACAGTGATTTCAGGTTTTACTGGGATTTAATAATGCTTATAATGATGGTT | | |
| | GGAAATCTAGTCATCATACCAGTTGGAATCACATTCTTTACAGAGCAAACAACAACAC | | |
| | CATGGATTATTTTCAATGTGGCATCAGATACAGTTTTCCTATTGGACCTGATCATGAA | | |
| | TTTTAGGACTGGGACTGTCAATGAAGACAGTTCTGAAATCATCCTGGACCCCAAAGTG | | |
| | ATCAAGATGAATTATTTAAAAAGCTGGTTTGTGGTTGACTTCATCTCATCCATCCCAG | | |
| | TGGATTATATCTTTCTTATTGTAGAAAAAGGAATGGATTCTGAAGTTTACAAGACAGC | | |
| | CAGGGCACTTCGCATTGTGAGGTTTACAAAAATTCTCAGTCTCTTGCGTTTATTACGA | | |
| | CTTTCAAGGTTAATTAGATACATACATCAATGGGAAGAGATATTCCACATGACATATG | | |
| | ATCTCGCCAGTGCAGTGGTGAGAATTTTTAATCTCATCGGCATGATGCTGCTCCTGTG | | |
| | CCACTGGGATGGTTGTCTTCAGTTCTTAGTACCACTACTGCAGGACTTCCCACCAGAT | | |
| | TGCTGGGTGTCTTTAAATGAAATGGTTAATGATTCTTGGGGAAAGCAGTATTCATACG | | |
| | CACTCTTCAAAGCTATGAGTCACATGCTGTGCATTGGGTATGGAGCCCAAGCCCCAGT | | |
| | CAGCATGTCTGACCTCTGGATTACCATGCTGAGCATGATCGTCGGGGCCACCTGCTAT | | |
| | GCCATGTTTGTCGGCCATGCCACCGCTTTAATCCAGTCTCTGGATTCTTCGAGGCGGC | | |
| | AGTATCAAGAGAAGTATAAGCAAGTGGAACAATACATGTCATTCCATAAGTTACCAGC | | |
| | TGATATGCGTCAGAAGATACATGATTACTATGAACACAGATACCAAGGCAAAATCTTT | | |
| | GATGAGGAAAATATTCTCAATGAACTCAATGATCCTCTGAGAGAGGAGATAGTCAACT | | |
| | TCAACTGTCGGAAACTGGTGGCTACAATGCCTTTATTTGCTAATGCGGATCCTAATTT | | |
| | TGTGACTGCCATGCTGAGCAAGTTGAGATTTGAGGTGTTTCAACCTGCAGATTATATC | | |
| | ATACGAGAAGGAGCCGTGGGTAAAAAAATGTATTTCATTCAACACGGTGTTGCTGGTG | | |
| | TCATTACAAAATCCAGTAAAGAAATGAAGCTGACAGATGGCTCTTACTTTGGGGAGAT | | |
| | TTGCCTGCTGACCAAAGGACGTCGTACTGCCAGTGTTCGAGCTGATACATATTGTCGT | | |
| | CTTTACTCACTTTCCCTGGACAATTTCAACGAGGTCCTGGAGGAATATCCAATGATGA | | |
| | GGAGAGCCTTTGAGACAGTTGCCATTGACCGACTAGATCGAATAGGAAAGAAAAATTC | | |
| | AATTCTTCTGCAAAAGTTCCAGAAGGATCTGAACACTGGTGTTTTCAACAATCAGGAG | | |
| | AACGAAATCCTCAAGCAGATTGTGAAACATGACAGGGAGATGGTGCAGGCAATCGCTC | | |
| | CCATCAATTATCCTCAAATGACAACCCTGAATTCCACATCGTCTACTACGACCCCGAC | | |
| | CTCCCGCATGAGGACACAATCTCCACCGGTGTACACAGCGACCAGCCTCTCTCACAGC | | |
| | AACCTGCACTCCCCCAGTCCCAGCACACAGACCCCCCAGCCATCAGCCATCCTGTCAC | | |
| | CCTGCTCCTACACCACCGCGGTCTGCAGCCCTCCTGTACAGAGCCCTCTGGCCGCTCG | | |
| | AACTTTCCACTATGCCTCCCCCACCGCCTCCCAGCTGTCACTCATGCAACAGCAGCCG | | |
| | CAGCAGCAGGTACAGCAGTCCCAGCCGCCGCAGACTCAGCCACAGCAGCCGTCCCCGC | | |
| | AGCCACAGACACCTGGCAGCTCCACGCCGAAAAATGAAGTGCACAAGAGCACGCAGGC | | |
| | GCTTCACAACACCAACCTGACCCGGGAAGTCAGGCCACTCTCCGCCTCGCAGCCCTCG | | |
| | CTGCCCCATGAGGTGTCCACTCTGATTTCCAGACCTCATCCCACTGTGGGCGAGTCCC | | |
| | TGGCCTCCATCCCTCAACCCGTGACGGCGGTCCCCGGAACGGGCCTTCAGGCAGGGGG | | |
| | CAGGAGCACTGTCCCGCAGCGCGTCACCCTCTTCCGACAGATGTCGTCGGGAGCCATC | | |
| | CCCCCGAACCGAGGAGTCCCTCCAGCACCCCCTCCACCAGCAGCTGCTCTTCCAAGAG | | |
| | AATCTTCCTCAGTCTTAAACACAGACCCAGACGCAGAAAAGCCACGATTTGCTTCAAA | | |
| | TTTATGATCCCTGCTGATTGTCAAAGCAGAAAGAAATACTCTCATAAACTGAGACTAT | | |
| | ACTCAGATCTTATTTTATTCTATCTCCTGATAGATCCCTCTAGCCTACTATGAAGAGA | | |
| | TATTTTAGACAGCTGTGGCCTACACGTGAAATGTAAAAATATATATACATATACTATA | | |
| | AAATATATATCTAAATTCCCAAGAGAGGGTCAAAAGACCTGTTTAGCATTCAGTGTTA | | |
| | TATGTCTTCCTTTCTTTAAATCATTAAAGGAT | | |
| | ORF Start: ATG at 61 | | ORF Stop: TGA at 2731 |
| | SEQ ID NO:352 | 890 aa | MW at 98791.0 kD |

| | |
|---|---|
| NOV42a, | MEGGGKPNSSSNSRDDGNSVFPAKASAPGAGPAAAEKRLGTPPGGGGAGAKEHGNSVC |
| CG159130-01 | |
| Protein Sequence | FKVDGGGGGGGGGGGGEEPAGGFEDAEGPRRQYGFMQRQFTSMLQPGVNKFSLRMFGS |
| | QKAVEKEQERVKTAGFWIIHPYSDFRFYWDLIMLIMMVGNLVIIPVGITFFTEQTTTP |
| | WIIFNVASDTVFLLDLIMNFRTGTVNEDSSEIILDPKVIKMNYLKSWFVVDFISSIPV |
| | DYIFLIVEKGMDSEVYKTARALRIVRFTKILSLLRLLRLSRLIRYIHQWEEIFHMTYD |
| | LASAVVRIFNLIGMMLLLCHWDGCLQFLVPLLQDFPPDCWVSLNEMVNDSWGKQYSYA |
| | LFKAMSHMLCIGYGAQAPVSMSDLWITMLSMIVGATCYAMFVGHATALIQSLDSSRRQ |
| | YQEKYKQVEQYMSFHKLPADMRQKIHDYYEHRYQGKIFDEENILNELNDPLREEIVNF |
| | NCRKLVATMPLFANADPNFVTAMLSKLRFEVFQPGDYIIREGAVGKKMYFIQHGVAGV |
| | ITKSSKEMKLTDGSYFGEICLLTKGRRTASVRADTYCRLYSLSVDNFNEVLEEYPMMR |
| | RAFETVAIDRLDRIGKKNSILLQKFQKDLNTGVFNNQENEILKQIVKHDREMVQAIAP |
| | INYPQMTTLNSTSSTTTPTSRMRTQSPPVYTATSLSHSNLHSPSPSTQTPQPSAILSP |
| | CSYTTAVCSPPVQSPLAARTFHYASPTASQLSLMQQQPQQQVQQSQPPQTQPQQPSPQ |
| | PQTPGSSTPKNEVHKSTQALHNTNLTREVRPLSASQPSLPHEVSTLISRPHPTVGESL |
| | ASIPQPVTAVPGTGLQAGGRSTVPQRVTLFRQMSSGAIPPNRGVPPAPPPPAAALPRE |
| | SSSVLNTDPDAEKPRFASNL |

Further analysis of the NOV42a protein yielded the following properties shown in Table 42B.

TABLE 42B
Protein Sequence Properties NOV42a

| | |
|---|---|
| PSort | 0.6000 probability located in plasma membrane; 0.4000 |
| analysis: | probability located in Golgi body; 0.3000 probability |
| | located in endoplasmic reticulum (membrane); |
| | 0.3000 probability located in microbody (peroxisome) |
| SignalP | No Known Signal Sequence Predicted |
| analysis: | |

A search of the NOV42a protein against the Geneseq database, a proprietary database that contains sequences published in patents and patent publication, yielded several homologous proteins shown in Table 42C.

TABLE 42C
Geneseq Results for NOV42a

| | | | Identities/ | |
|---|---|---|---|---|
| | | | Similarities for | |
| Geneseq | Protein/Organism/Length | NOV42a Residues/ | the Matched | Expect |
| Identifier | [Patent #, Date] | Match Residues | Region | Value |
| AAU11712 | Human HCN1 channel subunit full | 1 . . . 890 | 890/890 (100%) | 0.0 |
| | length sequence from splice variant | 1 . . . 890 | 890/890 (100%) | |
| | #1 - Homo sapiens, 890 aa. | | | |
| | [WO200190142-A2, 29 NOV. | | | |
| | 2001] | | | |
| AAU11714 | Human full length HCN1 channel | 1 . . . 890 | 888/890 (99%) | 0.0 |
| | subunit variant 2 - Homo sapiens, | 1 . . . 890 | 888/890 (99%) | |
| | 890 aa. [WO200190142-A2, 29 | | | |
| | NOV. 2001] | | | |
| AAE18675 | Human hyperpolarisation-activated | 1 . . . 890 | 885/890 (99%) | 0.0 |
| | cyclic nucleotide-gated channel 1 - | 1 . . . 890 | 885/890 (99%) | |
| | Homo sapiens, 890 aa. | | | |
| | [WO200202630-A2, 10 JAN. 2002] | | | |
| AAE21167 | Human TRICH-11 protein - Homo | 1 . . . 890 | 882/890 (99%) | 0.0 |
| | sapiens, 882 aa. [WO200212340- | 1 . . . 882 | 882/890 (99%) | |
| | A2, 14 FEB. 2002] | | | |
| AAY22191 | Mouse brain CNG-1 protein sequence - | 1 . . . 890 | 845/922 (91%) | 0.0 |
| | Mus sp, 910 aa. [WO9932615-A1, | 1 . . . 910 | 852/922 (91%) | |
| | 01 JUL. 1999] | | | |

In a BLAST search of public sequence datbases, the NOV42a protein was found to have homology to the proteins shown in the BLASTP data in Table 42D.

TABLE 42D
Public BLASTP Results for NOV42a

| | | | Identities/ | |
|---|---|---|---|---|
| Protein | | | Similarities for | |
| Accession | | NOV42a Residues/ | the Matched | Expect |
| Number | Protein/Organism/Length | Match Residues | Portion | Value |
| O88704 | Hyperpolarization-activated cation | 1 . . . 890 | 846/922 (91%) | 0.0 |
| | channel, HAC2 - Mus musculus | 1 . . . 910 | 853/922 (91%) | |
| | (Mouse), 910 aa. | | | |
| Q9JKB0 | Hyperpolarization-activated, cyclic | 1 . . . 890 | 847/922 (91%) | 0.0 |
| | nucleotide-gated potassium channel | 1 . . . 910 | 856/922 (91%) | |
| | 1 - Rattus norvegicus (Rat), 910 aa. | | | |
| O54899 | Brain cyclic nucleotide gated 1 - | 1 . . . 890 | 845/922 (91%) | 0.0 |
| | Mus musculus (Mouse), 910 aa. | 1 . . . 910 | 852/922 (91%) | |
| Q9MZS1 | Hyperpolarization-activated cyclic | 78 . . . 890 | 786/813 (96%) | 0.0 |
| | nucleotide-gated channel 1 - | 14 . . . 822 | 792/813 (96%) | |
| | Oryctolagus cuniculus (Rabbit), | | | |
| | 822 aa. | | | |
| O60741 | Ion channel BCNG-1 - Homo | 122 . . . 870 | 737/749 (98%) | 0.0 |
| | sapiens (Human), 749 aa | 1 . . . 749 | 739/749 (98%) | |
| | (fragment). | | | |

PFam analysis predicts that the NOV42a protein contains the domains shown in the Table 42E.

TABLE 42E
Domain Analysis of NOV42a

| | | Identities/ | |
|---|---|---|---|
| | | Similarities for | |
| Pfam | NOV42a | the Matched | Expect |
| Domain | Match Region | Region | Value |
| ion_trans | 174 . . . 393 | 50/244 (20%) | 1.6e−22 |
| | | 160/244 (66%) | |
| cNMP_binding | 490 . . . 578 | 31/120 (26%) | 2e−28 |
| | | 71/120 (59%) | |
| Transthyretin | 692 . . . 709 | 12/19 (63%) | 0.82 |
| | | 14/19 (74%) | |

### Example 43

The NOV43 clone was analyzed, and the nucleotide and encoded polypeptide sequences are shown in Table 43A.

TABLE 43A
NOV43 Sequence Analysis

| | SEQ ID NO:353 | 1136 bp | |
|---|---|---|---|
| NOV43a, | AACACCATGAGGGCCCTGGTGCTTCTGCTGTCCCTGTTCCTGCTGGGTGGCCAGGCCC | | |
| CG159178-01 | | | |
| DNA Sequence | AGCATGTGTCTGACTGGACCTACTCAGTGCAGATCGGCCTGCCCTCCACCATGCGCAT | | |
| | GACAGTGGCTGACGGCACTGTATACGTAGCCCAGCAGATGCACTTTCACTGGGGAGGT | | |
| | GCGTCCTCGGAGATCAGCGGCTCTGAGCACACCGTGGACGGGATCAGACATGTGATCG | | |
| | AGATTCACATTGTTCACTACAATTCTAAATACAAGAGCTATGATATAGCCCAAGATGC | | |
| | GCCGGATGGTTTGGCTGTACTGGCAGCCTTCGTTGAGGTGAAGAATTACCCTGAAAAC | | |
| | ACTTATTACAGCAACTTCATTTCTCATCTGGCCAACATCAAGTACCCAGGACAAAGAA | | |
| | CAACCCTGACTGGCCTTGACGTTCAGGACATGCTGCCCAGGAACCTCCAGCACTACTA | | |
| | CACCTACCATGGCTCACTCACCACGCCTCCCTGCACTGAGAACGTCCACTGGTTTGTG | | |
| | CTGGCAGATTTTGTCAAGCTCTCCAGGACACAGGTTTGGAAGCTGGAGAATTCCTTAC | | |
| | TGGATCACCGCAATAAGACCATCCACAACGATTACCGCAGGACCCAGCCCCTGAAACA | | |
| | CAGAGTGGTGGAATCCAACTTCCCGAATCAGGAATACACTCTAGGCTCTGAATTCCAG | | |
| | TTTTACCTACATAAGATTGAGGAAATTCTTGACTACTTAAGAAGAGCATTGAACTGAG | | |
| | GAAAGCTAAGAGGAAGATTCAATAATATTAACTAGCTTGAAGCCTGACCTAGCCAGAA | | |
| | GTGCCTGTCCGCTGCAGCCGCACCCTACCTTGTCTAAGAAACCATGTGTGTCTGGAAC | | |
| | ACGCTGCTCCCCTGGGCAGCTGTTGGGATTCTGATTAAAGAGGGGAAACGATCATCCT | | |
| | GGACAGGAAGTGAGATGGCTTCAGTTCATGAGACGGGATCTGAGTTAGACATCACCAG | | |
| | TGGAAATTGATTGGAATAGAAACTTAAAGGAAATGGAACCCTAACTATTCTCCCATCA | | |
| | AATCATATATGTTGACCTGTCTGAATTATAAACCAGCCTGACCTTTCCTTTAGCATTA | | |
| | GATGTAATAAAATAACTTTGGAAATTTGTCATTT | | |
| | ORF Start: ATG at 7 | | ORF Stop: TGA at 751 |
| | SEQ ID NO:354 | 248 aa | MW at 28657.2 kD |
| NOV43a, | MRALVLLLSLFLLGGQAQHVSDWTYSVQIGLPSTMRMTVADGTVYVAQQMHFHWGGAS | | |
| CG159178-01 | | | |
| Protein Sequence | SEISGSEHTVDGIRHVIEIEIVHYNSKYKSYDIAQDAPDGLAVLAAFVEVKNYPENTY | | |
| | YSNFISHLANIKYPGQRTTLTGLDVQDMLPRNLQHYYTYHGSLTTPPCTENVHWFVLA | | |
| | DFVKLSRTQVWKLENSLLDHRNKTIHNDYRRTQPLKHRVVESNFPNQEYTLGSEFQFY | | |
| | LHKIEEILDYLRRALN | | |
| | SEQ ID NO:355 | 1006 bp | |
| NOV43b, | AACACCATGAGGGCCCTGGTGCTTCTGCTGTCCCTGTTCCTGCTGGGTGGCCAGGCCC | | |
| CG159178-02 | | | |
| DNA Sequence | AGCATGTGTCTGACTGGACCTACTCAGAAGGGGCACTGGACGAAGCGCACTGGCCACA | | |
| | GCACTACCCCGCCTGTGGGGGCCAGAGACAGTCGCCTATCAACCTACAGAGGACGAAG | | |
| | GTGCGGTACAACCCCTCCTTGAAGGGGCTCAATATGACAGGCTATGAGACCCAGGCAG | | |
| | GGGAGTTCCCCATGGTCAACAATGGCCACACAGTGCACATCGGCCTGCCCTCCACCAT | | |
| | GCGCATGACAGTGGCTGACGGCACTGTATACATAGCCCAGCAGATGCACTTTCACTGG | | |
| | GGAGGTGCGTCCTCGGAGATCAGCGGCTCTGAGCACACCGTGGACGGGATCAGACATG | | |
| | TGATCGAGATTCACATTGTTCACTACAATTCTAAATACAAGAGCTATGATATAGCCCA | | |
| | AGATGCGCCGGATGGTTTGGCTGTACTGGCAGCCTTCGTTGAGGTGAAGAATTACCCT | | |
| | GAAAACACTTATTACAGCAACTTCATTTCTCATCTGGCCAACATCAAGTACCCAGGAC | | |
| | AAAGAACAACCCTCACTGGCCTTGACGTTCAGGACATGCTGCCCAGGAACCTCCAGCA | | |
| | CTACTACACCTACCATGGCTCACTCACCACGCCTCCCTGCACTGAGAACGTCCACTGG | | |
| | TTTGTGCTGGCAGATTTTGTCAAGCTCTCCAGGACACAGGTTTGGAAGCTGGAGAATT | | |
| | CCTTACTGGATCACCGCAATAAGACCATCCACAACGATTACCGCAGGACCCAGCCCCT | | |
| | GAACCACAGAGTGGTGGAATCCAACTTCCCGAATCAGGAATACACTCTAGGCTCTGAA | | |
| | TTCCAGTTTTACCTACATAAGATTGAGGAAATTCTTGACTACTTAAGAAGAGCATTGA | | |
| | ACTGAGGAAAGCTAAGAGGAAGATTCAATATTAACTAGCTTGAAGCCTGACCTAGCCA | | |
| | AGGGCGATTCCACACACTCC | | |
| | ORF Start: ATG at 7 | | ORF Stop: TGA at 931 |
| | SEQ ID NO:356 | 308 aa | MW at 35336.5 kD |

| | |
|---|---|
| NOV43b, | MRALVLLLSLFLLGGQAQHVSDWTYSEGALDEAHWPQHYPACGGQRQSPINLQRTKVR |
| CG159178-02 | |
| Protein Sequence | YNPSLKGLNMTGYETQAGEFPMVNNGHTVQIGLPSTMRMTVADGTVYIAQQMHFHWGG |
| | ASSEISGSEHTVDGIRHVIEIHIVHYNSKYKSYDIAQDAPDGLAVLAAFVEVKNYPEN |
| | TYYSNFISHLANIKYPGQRTTLTGLDVQDMLPRNLQHYYTYHGSLTTPPCTENVHWFV |
| | LADFVKLSRTQVWKLENSLLDHRNKTIHNDYRRTQPLNHRVVESMFPNQEYTLGSEFQ |
| | FYLHKIEEILDYLRRALN |

Sequence comparison of the above protein sequences yields the following sequence relationships shown in Table 43B.

TABLE 43B
Comparison of NOV43a against NOV43b.

| | | Identities/ |
|---|---|---|
| | | Similarities for |
| Protein | NOV43a Residues/ | the Matched |
| Sequence | Match Residues | Region |
| NOV43b | 25 . . . 248 | 220/224 (98%) |
| | 85 . . . 308 | 223/224 (99%) |

Further analysis of the NOV43a protein yielded the following properties shown in Table 43C.

TABLE 43C
Protein Sequence Properties NOV43a

| | |
|---|---|
| PSort | 0.4132 probability located in outside; 0.2473 probability |
| analysis: | located in microbody (peroxisome); 0.1000 probability |
| | located in endoplasmic reticulum (membrane); 0.1000 |
| | probability located in endoplasmic reticulum (lumen) |
| SignalP | Cleavage site between residues 18 and 19 |
| analysis: | |

A search of the NOV43a protein against the Geneseq database, a proprietary database that contains sequences published in patents and patent publication, yielded several homologous proteins shown in Table 43D.

TABLE 43D
Geneseq Results for NOV43a

| | | | Identities/ | |
|---|---|---|---|---|
| | | | Similarities for | |
| Geneseq | Protein/Organism/Length | NOV43a Residues/ | the Matched | Expect |
| Identifier | [Patent #, Date] | Match Residues | Region | Value |
| AAB59592 | Human carbonic anhydrase isoform | 25 . . . 219 | 189/195 (96%) | e−112 |
| | #5 - Homo sapiens, 262 aa. | 68 . . . 262 | 193/195 (98%) | |
| | [US6160090-A, 12 DEC. 2000] | | | |
| AAE17175 | Human RCC-associated antigen, | 25 . . . 219 | 82/195 (42%) | 3e−37 |
| | G250 protein - Homo sapiens, 459 | 200 . . . 391 | 112/195 (57%) | |
| | aa. [WO200198363-A2, 27 DEC. | | | |
| | 2001] | | | |
| AAB82848 | Kidney cancer specific antigen | 25 . . . 219 | 82/195 (42%) | 3e−37 |
| | G250-GM-CSF fusion protein - | 345 . . . 536 | 112/195 (57%) | |
| | Homo sapiens, 610 aa. | | | |
| | [WO200160317-A2, 23 AUG. 2001] | | | |
| AAY53245 | MN protein extracellular domain | 25 . . . 219 | 82/195 (42%) | 3e−37 |
| | SEQ ID NO: 87 - Homo sapiens, 377 | 163 . . . 354 | 112/195 (57%) | |
| | aa. [US6027887-A, 22 FEB. 2000] | | | |
| AAY53241 | MN protein carbonic anhydrase | 25 . . . 219 | 82/195 (42%) | 3e−37 |
| | domain SEQ ID NO: 51 - Homo | 66 . . . 257 | 112/195 (57%) | |
| | sapiens, 257 aa. [US6027887-A, 22 | | | |
| | FEB. 2000] | | | |

In a BLAST search of public sequence datbases, the NOV43a protein was found to have homology to the proteins shown in the BLASTP data in Table 43E.

TABLE 43E
Public BLASTP Results for NOV43a

| | | | Identities/ | |
|---|---|---|---|---|
| Protein | | | Similarities for | |
| Accession | | NOV43a Residues/ | the Matched | Expect |
| Number | Protein/Organism/Length | Match Residues | Portion | Value |
| P23280 | Carbonic anhydrase VI precursor (EC | 25 . . . 248 | 220/224 (98%) | e−131 |
| | 4.2.1.1) (Carbonate dehydratase VI) | 85 . . . 308 | 224/224 (99%) | |
| | (CA-VI) (Secreted carbonic | | | |
| | anhydrase) (Salivary carbonic | | | |
| | anhydrase) - Homo sapiens (Human), | | | |
| | 308 aa. | | | |
| Q96QX8 | DJ477M7.5 (carbonic anhydrase VI) - | 25 . . . 248 | 219/224 (97%) | e−130 |
| | Homo sapiens (Human), 308 aa. | 85 . . . 308 | 222/224 (98%) | |
| CRHU6 | carbonate dehydratase (EC 4.2.1.1) | 25 . . . 248 | 218/224 (97%) | e−129 |
| | VI precursor - human, 308 aa. | 85 . . . 308 | 222/224 (98%) | |
| A29993 | carbonate dehydratase (EC 4.2.1.1) | 25 . . . 245 | 164/224 (73%) | 1e−94 |
| | VI - sheep, 307 aa. | 68 . . . 291 | 193/224 (85%) | |
| E966553 | SYNTHETIC OVINE CARBONIC | 25 . . . 245 | 164/224 (73%) | 1e−94 |
| | ANHYDRASE VI PROTEIN - | 68 . . . 291 | 193/224 (85%) | |
| | vectors, 307 aa. | | | |

PFam analysis predicts that the NOV43a protein contains the domains shown in the Table 43F.

TABLE 43F
Domain Analysis of NOV43a

| | | Identities/ | |
|---|---|---|---|
| | | Similarities for | |
| Pfam | NOV43a | the Matched | Expect |
| Domain | Match Region | Region | Value |
| Carb_anhydrase | 25 . . . 218 | 86/210 (41%) | 1.6e−118 |
| | | 191/210 (91%) | |

### Example 44

The NOV44 clone was analyzed, and the nucleotide and encoded polypeptide sequences are shown in Table 44A.

TABLE 44A
NOV44 Sequence Analysis

| | SEQ ID NO:357 | 1704 bp | |
|---|---|---|---|
| NOV44a, | GGTTTCATGGCAGCCTCAAAGAAGGCAGTTTTGGGGCCATTGGTGGGGGCGGTGGACC | | |
| CG160131-01 | | | |
| DNA Sequence | AGGGCACCAGTTCGACGCGCTTTTTGGTTTTCAATTCAAAAACAGCTGAACTACTTAG | | |
| | TCATCATCAAGTAGAAATAAAACAAGAGTTCCCAAGAGAAGGATGGGTGGAACAGGAC | | |
| | CCTAAGGAAATTCTACATTCTGTCTATGAGTGTATAGAGAAAACATGTGAGAAACTTG | | |
| | GACAGCTCAATATTGATATTTCCAACATAAAAGCTATTGGTGTCAGCAACCAGAGGGA | | |
| | AACCACTGTAGTCTGGGACAAGATAACTGGAGAGCCTCTCTACAATGCTGTGGCTGCT | | |
| | CCAGTTTCTCCTGGCCCTTCAGTTCCAGTTGCCGTTGTTCCCTCTGGCTCTTCAGTTC | | |
| | CAGCTCCTGGTACTTCCTCAGTGTGGCTTGATCTAAGAACCCAGTCTACCGTTGAGAG | | |
| | TCTTAGTAAAAGAATTCCAGGAAATAATAACTTTGTCAAGTCCAAGACAGGCCTTCCA | | |
| | CTTAGCACTTACTTCAGTGCAGTGAAACTTCGTTGGCTCCTTGACAATGTGAGAAAAG | | |
| | TTCAAAAGGCCGTTGAAGAAAAACGAGCTCTTTTTGGGACTATTGATTCATGGCTTAT | | |
| | TTGGAGTTTGACAGGAGGAGTCAATGGAGGTGTCCACTGTACAGATGTAACAAATGCA | | |
| | AGTAGGACTATGCTTTTCAACATTCATTCTTTGGAATGGGATAAACAACTCTGCGAAT | | |
| | TTTTTGGAATTCCAATGGAAATTCTTCCAAATGTCCGGAGTTCTTCTGAGATCTATGG | | |
| | CCTAATGAAAGCTGGGGCCTTGGAAGGTGTGCCAATATCTGGGTGTTTAGGGGACCAG | | |
| | TCTGCTGCATTGGTGGGACAAATGTGCTTCCAGATTGGACAAGCCAAAAATACGTATG | | |
| | GAACAGGATGTTTCTTACTATGTAATACAGGCCATAAGTGTGTATTTTCTGATCATGG | | |
| | CCTTCTCACCACAGTGGCTTACAAACTTGGCAGAGACAAACCAGTATATTATGCTTTG | | |
| | GAAGGTTCTGTAGCTATAGCTGGTGCTGTTATTCGCTGGCTAAGAGACAATCTTGGAA | | |
| | TTATAAAGACCTCAGAAGAAATTGAAAAACTTGCTAAAGAAGTAGGTACTTCTTATGG | | |
| | CTGCTACTTCGTCCCAGCATTTTCGGGGTTATATGCACCTTATTGGGAGCCCAGCGCA | | |
| | AGAGGGATAATCTGTGGACTCACTCAGTTCACCAATAAATGCCATATTGCTTTTGCTG | | |
| | CATTAGAAGCTGTTTGTTTCCAAACTCGAGAGATTTTGGATGCCATGAATCGAGACTG | | |
| | TGGAATTCCACTCAGTCATTTGCAGGTAGATGGAGGAATGACCAGCAACAAAATTCTT | | |
| | ATGCAGCTACAAGCAGACATTCTGTATATACCAGTAGTGAAGCCCTCAATGCCCGAAA | | |
| | CCACTGCACTGGGTGCGGCTATGGCGGCAGGGGCTGCAGAAGGAGTCGGCGTATGGAG | | |
| | TCTCGAACCCGAGGATTTGTCTGCCGTCACGATGGAGCGGTTTGAACCTCAGATTAAT | | |
| | GCGGAGGAAAGTGAAATTCGTTATTCTACATGGAAGAAAGCTGTGATGAAGTCAATGG | | |
| | GTTGGGTTACAACTCAATCTCCAGAAAGTGGTATTCCATAAAACCTACCAACTCATGG | | |
| | ATTCCCAAGATGTGAGCTTTTT | | |
| | ORF Start: ATG at 7 | | ORF Stop: TAA at 1663 |
| | SEQ ID NO:358 | 552 aa | MW at 59929.2 kD |
| NOV44a, | MAASKKAVLGPLVGAVDQGTSSTRFLVFNSKTAELLSNHQVEIKQEFPREGWVEQDPK | | |
| CG160131-01 | | | |
| Protein Sequence | EILHSVYECIEKTCEKLGQLNIDISNIKAIGVSNQRETTVVWDKITGEPLYNAVAAPV | | |
| | SPGPSVPVAVVPSGSSVPAPGTSSVWLDLRTQSTVESLSKRIPGNNNFVKSKTGLPLS | | |
| | TYFSAVKLRWLLDNVRKVQKAVEEKRALFGTIDSWLIWSLTGGVNGGVHCTDVTNASR | | |
| | TMLFNIHSLEWDKQLCEFFGIPMEILPNVRSSSEIYGLMKAGALEGVPISGCLGDQSA | | |
| | ALVGQMCFQIGQAKNTYGTGCFLLCNTGHKCVFSDHGLLTTVAYKLGRDKPVYYALEG | | |
| | SVAIAGAVIRWLRDNLGIIKTSEEIEKIAKEVGTSYGCYFVPAFSGLYAPYWEPSARG | | |
| | IICGLTQFTNKCHIAFAALEAVCFQTREILDAMNRDCGIPLSHLQVDGGMTSNKILMQ | | |
| | LQADILYIPVVKPSMPETTALGAAMAAGAAEGVGVWSLEPEDLSAVTMERFEPQINAE | | |
| | ESEIRYSTWKKAVMKSMGWVTTQSPESGIP | | |
| | SEQ ID NO:359 | 1609 bp | |
| NOV44b, | CACCGGATCCATGGCAGCCTCAAAGAAGGCAGTTTTGGGGCCATTGGTGGGGGCGGTG | | |
| CG160131-04 | | | |
| DNA Sequence | GACCAGGGCACCAGTTCGACGCGCTTTTTGGTTTTCAATTCAAAAACAGCTGAACTAC | | |
| | TTAGTCATCATCAAGTAGAAATAAAACAAGAGTTCCCAAGAGAAGGATGGGTGGAACA | | |
| | GGACCCTAAGGAAATTCTACATTCTGTCTATGAGTGTATAGAGAAAACATGTGAGAAA | | |
| | CTTGGACAGCTCAATATTGATATTTCCAACATAAAAGCTATTGGTGTCAGCAACCAGA | | |
| | GGGAAACCACTGTAGTCTGGGACAAGATAACTGGAGAGCCTCTCTACAATGCTGTGGT | | |
| | GTGGCTTGATCTAAGAACCCAGTCTACCGTTGAGAGTCTTAGTAAAAGAATTCCAGGA | | |
| | AATAATAACTTTGTCAAGTCCAAGACAGGCCTTCCACTTAGCACTTACTTCAGTGCAG | | |
| | TGAAACTTCGTTGGCTCCTTGACAATGTGAGAAAAGTTCAAAAGGCCGTTGAAGAAAA | | |
| | ACGAGCTCTTTTTGGGACTATTGATTCATGGCTTATTTGGAGTTTGACAGGAGGAGTC | | |
| | AATGGAGGTGTCCACTGTACAGATGTAACAAATGCAAGTAGGACTATGCTTTTCAACA | | |
| | TTCATTCTTTGGAATGGGATAAACAACTCTGCGAATTTTTTGGAATTCCAATGGAAAT | | |
| | TCTTCCAAATGTCCGGAGTTCTTCTGAGATCTATGGCCTAATGAAAATCTCTCATAGC | | |
| | GTGAAAGCTGGGGCCTTGGAAGGTGTGCCAATATCTGGGTGTTTAGGGGACCAGTCTG | | |
| | CTGCATTGGTGGGACAAATGTGCTTCCAGATTGGACAAGCCAAAAATACGTATGGAAC | | |
| | AGGATGTTTCTTACTATGTAATACAGGCCATAAGTGTGTATTTTCTGATCATGGCCTT | | |
| | CTCACCACAGTGGCTTACAAAAACTTGGCAGAGACAAACCAGTATATTATGCTTTGGG | | |
| | GTTCTGTAGCTATAGCTGGTGCTGTTATTCGCTGGCTAAGAGACAATCTTGGAATTAT | | |
| | AAAGACCTCAGAAGAAATTGAAAAACTTGCTAAAGAAGTAGGTACTTCTTATGGCTGC | | |
| | TACTTCGTCCCAGCATTTTCGGGGTTATATGCACCTTATTGGGAGCCCAGCGCAAGAG | | |
| | GGATAATCTGTGGACTCACTCAGTTCACCAATAAATGCCATATTGCTTTTGCTGCATT | | |
| | AGAAGCTGTTTGTTTCCAAACTCGAGAGATTTTGGATGCCATGAATCGAGACTGTGGA | | |
| | ATTCCACTCAGTCATTTGCAGGTAGATGGAGGAATGACCAGCAACAAAATTCTTATGC | | |
| | AGCTACAAGCAGACATTCTGTATATACCAGTAGTGAAGCCCTCAATGCCCGAAACCAC | | |
| | TGCACTGGGTGCGGCTATGGCGGCAGGGGCTGCAGAAGGAGTCGGCGTATGGAGTCTC | | |
| | GAACCCGAGGATTTGTCTGCCGTCACGATGGAGCGGTTTGAACCTCAGATTAATGCGG | | |
| | AGGAAAGTGAAATTCGTTATTCTACATGGAAGAAAGCTGTGATGAAGTCAATGGGTTG | | |
| | GGTTACAACTCAATCTCCAGAAAGTGGTATTCCAGTCGACGGC | | |
| | ORF Start: at 2 | | ORF Stop: end of |
| | | | sequence |
| | SEQ ID NO:360 | 536 aa | MW at 58656.8 kD |
| NOV44b, | TGSMAASKKAVLGPLVGAVDQGTSSTRFLVFNSKTAELLSHHQVEIKQEFPREGWVEQ | | |
| CG160131-04 | | | |
| Protein Sequence | DPKEILHSVYECIEKTCEKLGQLNIDISNIKAIGVSNQRETTVVWDKITGEPLYNAVV | | |
| | WLDLRTQSTVESLSKRIPGNNNFVKSKTGLPLSTYFSAVKLRWLLDNVRKVQKAVEEK | | |
| | RALFGTIDSWLIWSLTGGVNGGVHCTDVTNASRTMLFNIHSLEWDKQLCEFFGIPMEI | | |
| | LPNVRSSSEIYGLMKISHSVKAGALEGVPISGCLGDQSAALVGQMCFQIGQAKNTYGT | | |
| | GCFLLCNTGHKCVFSDHGLLTTVAYKLGRDKPVYYALEGSVAIAGAVIRWLRDNLGII | | |
| | KTSEEIEKLAKEVGTSYGCYFVPAFSGLYAPYWEPSARGIICGLTQFTNKCNTAFAAL | | |
| | EAVCFQTREILDAMNRDCGIPLSHLQVDGGMTSNKILMQLQADILYTPVVKPSMPETT | | |
| | ALGAAMAAGAAEGVGVWSLEPEDLSAVTMERFEPQINAEESEIRYSTWKKAVMKSMGW | | |
| | VTTQSPESGIPVDG | | |
| | SEQ ID NO:361 | 1581 bp | |
| NOV44c, | GGTTTCATGGCAGCCTCAAAGAAGGCAGTTTTGGGGCCATTGGTGGGGGCGGTGGACC | | |
| CG160131-02 | | | |
| DNA Sequence | AGGGCACCAGTTCGACGCGCTTTTTGGTTTTCAATTCAAAAACAGCTGAACTACTTAG | | |
| | TCATCATCAAGTAGAAATAAAACAAGAGTTCCCAAGAGAAGGATGGGTGGAACAGGAC | | |
| | CCTAAGGAAATTCTACATTCTGTCTATGAGTGTATAGAGAAAACATGTGAGAAACTTG | | |
| | GACAGCTCAATATTGATATTTCCAACATAAAAGCTATTGGTGTCAGCAACCAGAGGGA | | |
| | AACCACTGTAGTCTGGGACAAGATAACTGGAGAGCCTCTCTACAATGCTGTGGTGTGG | | |
| | CTTGATCTAAGAACCCAGTCTACCGTTGAGAGTCTTAGTAAAAGAATTCCAGGAAATA | | |
| | ATAACTTTGTCAAGTCCAAGACAGGCCTTCCACTTAGCACTTACTTCAGTGCAGTGAA | | |
| | ACTTCGTTGGCTCCTTGACAATGTGAGAAAAGTTCAAAAGGCCGTTGAAGAAAAACGA | | |
| | GCTCTTTTTGGGACTATTGATTCATGGCTTATTTGGAGTTTGACAGGAGGAGTCAATG | | |
| | GAGGTGTCCACTGTACAGATGTAACAAATGCAAGTAGGACTATGCTTTTCAACATTCA | | |
| | TTCTTTGGAATGGGATAAACAACTCTGCGAATTTTTTGGAATTCCAATGGAAATTCTT | | |
| | CCAAATGTCCGGAGTTCTTCTGAGATCTATGGCCTAATGAAAGCTGGGGCCTTGGAAG | | |
| | GTGTGCCAATATCTGGGTGTTTAGGGGACCAGTCTGCTGCATTGGTGGGACAAATGTG | | |
| | CTTCCAGATTGGACAAGCCAAAAATACGTATGGAACAGGATGTTTCTTACTATGTAAT | | |
| | ACAGGCCATAAGTGTGTATTTTCTGATCATGGCCTTCTCACCACAGTGGCTTACAAAC | | |
| | TTGGCAGAGACAAACCAGTATATTATGCTTTGGAAGGTTCTGTAGCTATAGCTGGTGC | | |
| | TGTTATTCGCTGGCTAAGAGACAATCTTGGAATTATAAAGACCTCAGAAGAAATTGAA | | |
| | AAACTTGCTAAAGAAGTAGGTACTTCTTATGGCTGCTACTTCGTCCCAGCATTTTCGG | | |
| | GGTTATATGCACCTTATTGGGAGCCCAGCGCAAGAGGGATAATCTGTGGACTCACTCA | | |
| | GTTCACCAATAAATGCCATATTGCTTTTGCTGCATTAGAAGCTGTTTGTTTCCAAACT | | |
| | CGAGAGATTTTGGATGCCATGAATCGAGACTGTGGAATTCCACTCAGTCATTTGCAGG | | |
| | TAGATGGAGGAATQACCAGCAACAAAATTCTTATGCAGCTACAAGCAGACATTCTGTA | | |
| | TATACCAGTAGTGAAGCCCTCAATGCCCGAAACCACTGCACTGGGTGCGGCTATGGCG | | |
| | GCAGGGGCTGCAGAAGGAGTCGGCGTATGGAGTCTCGAACCCGAGGATTTGTCTGCCG | | |
| | TCACGATGGAGCGGTTTGAACCTCAGATTAATGCGGACGAAAGTGAAATTCGTTATTC | | |
| | TACATGGAAGAAAGCTGTGATGAAGTCAATGGGTTGGGTTACAACTCAATCTCCAGAA | | |
| | AGTGGTATTCCATAA | | |
| | ORF Start: ATG at 7 | | ORF Stop: TAA at 1579 |
| | SEQ ID NO:362 | 524 aa | MW at 57488.5 kD |
| NOV44c, | MAASKKAVLGPLVGAVDQGTSSTRFLVFNSKTAELLSHHQVEIKQEFPREGWVEQDPK | | |
| CG160131-02 | | | |
| Protein Sequence | EILHSVYECIEKTCEKLGQLNIDISNIKAIGVSNQRETTVVWDKITGEPLYNAVVWLD | | |
| | LRTQSTVESLSKRIPGNNNFVKSKTGLPLSTYFSAVKLRWLLDNVRKVQKAVEEKRAL | | |
| | FGTIDSWLIWSLTGGVNGGVHCTDVTNASRTMLFNIHSLEWDKQLCEFFGIPMEILPN | | |
| | VRSSSEIYGLMKAGALEGVPISGCLGDQSAALVGQMCFQIGQAKNTYGTGCFLLCNTG | | |
| | HKCVFSDHGLLTTVAYKLGRDKPVYYALEGSVAIAGAVIRWLRDNLGIIKTSEEIEKL | | |
| | AKEVGTSYGCYFVPAFSGLYAPYWEPSARGIICGLTQFTNKCHIAFAALEAVCFQTRE | | |
| | ILDANNRDCGIPLSHLQVDGGMTSNKILMQLQADILYIPVVKPSMPETTALGAAMAAG | | |
| | AAEGVGVWSLEPEDLSAVTMERFEPQINAEESEIRYSTWKKAVMKSMGWVTTQSPESG | | |
| | IP | | |
| | SEQ ID NO:363 | 1625 bp | |
| NOV44d, | TCGCCCTTTTGACTGTATCGCCGGAATTCATGGCAGCCTCAAAGAAGGCAGTTTTGGG | | |
| CG160131-03 | | | |
| DNA Sequence | GCCATTGGTGGGGGCGGTGGACCAGGCCACCAGTTCGACGCGCTTTTTGGTTTTCAAT | | |
| | TCAAAAACAGCTGAACTACTTAGTCATCATCAAGTAGAAATAAAACAAGAGTTCCCAA | | |
| | GAGAAGGATGGGTGGAACAGGACCCTAAGGAAATTCTACATTCTGTCTATGAGTGTAT | | |
| | AGAGAAAACATGTGAGAAACTTGGACAGCTCAATATTGATATTTCCAACATAAAAGCT | | |
| | ATTGGTGTCAGCAACCAGAGGGAAACCACTGTAGTCTGGGACAAGATAACTGGAGAGC | | |
| | CTCTCTACAATGCTGTGGTGTGGCTTGATCTAAGAACCCAGTCTACCGTTGAGAGTCT | | |
| | TAGTAAAAGAATTCCAGGAAATAATAACTTTGTCAAGTCCAAGACAGGCCTTCCACTT | | |
| | AGCACTTACTTCAGTGCAGTGAAACTTCGTTGGCTCCTTGACAATGTGAGAAAAGTTC | | |
| | AAAAGGCCGTTGAAGAAAAACGAGCTCTTTTTGGGACTATTGACTCATGGCTTATTTG | | |
| | GAGTTTGACAGGAGGAGTCAATGGAGGTGTCCACTGTACAGATGTAACAAATGCAAGT | | |
| | AGGACTATGCTTTTCAACATTCATTCTTTGGAATGGGATAAACAACTCTGCGAATTTT | | |
| | TTGGAATTCCAATGGAAATTCTTCCAAATGTCCGGAGTTCTTCTGAGATCTATGGCCT | | |
| | AATGAAAGCTGGGGCCTTGGAAGGTGTGCCAATATCTGGGTGTTTAGGGGACCAGTCT | | |
| | GCTGCATTGGTGGGACAAATGTGCTTCCAGATTGGACAAGCCAAAAATACGTATGGAA | | |
| | CAGGATGTTTCTTACTATGTAATACAGGCCATAAGTGCGTATTTTCTGATCATGGCCT | | |
| | TCTCACCACAGTGGCTTACAAACTTGGCAGAGACAAACCAGTATATTATGCTTTGGAA | | |
| | GGTTCTGTAGCTATAGCTGGTGCTGTTATTCGCTGGCTAAGAGACAATCTTGGAATTA | | |
| | TAAAGACCTCAGAAGAAATTGAAAAACTTGCTAAAGAAGTAGGTACTTCTTATGGCTG | | |
| | CTACTTCGTCCCAGCATTTTCGGGGTTATATGCACCTTATTGGGAGCCCAGCGCAAGA | | |
| | GGGATAATCTGTGGACTCACTCAGTTCACCAATAAATGCCATATTGCTTTTGCTGCAT | | |
| | TAGAAGCTGTTTGTTTCCAAACTCGAGAGATTTTGGATGCCATGAATCGAGACTGTGG | | |
| | AATTCCACTCAGTCATTTGCAGGTAGATGGAGGAATGACCAGCAACAAAATTCTTATG | | |
| | CAGCTACAAGCAGACATTCTGTATATACCAGTAGTGAAGCCCTCAATGCCCGAAACCA | | |
| | CTGCACTGGGTGCGGCTATGGCGGCAGGGGCTGCAGAAGGAGTCGGCGTATGGAGTCT | | |
| | CGAACCCGAGGATCTGTCTGCCGTCACGATGGAGCGGTTTGAACCTCAGATTAATGCG | | |
| | GAGGAAAGTGAAATTCGTTATTCTACATGGAAGAAAGCTGTGATGAAGTCAATGGGTT | | |
| | GGGTTACAACTCAATCTCCAGAAAGTGGTATTCCATGACTGCAGCCAACCTAATTCCG | | |
| | ORF Start: ATG at 30 | | ORF Stop: TGA at 1602 |
| | SEQ ID NO:364 | 524 aa | MW at 57502.5 kD |

| | |
|---|---|
| NOV44d, | MAASKKAVLGPLVGAVDQATSSTRFLVFNSKTAELLSHHQVEIKQEFPREGWVEQDPK |
| CG160131-03 | |
| Protein Sequence | EILHSVYECIEKTCEKLGQLNIDISNIKAIGVSNQRETTVVWDKITGEPLYNAVVWLD |
| | LRTQSTVESLSKRIPGNNNFVKSKTGLPLSTYFSAVKLRWLLDNVRKVQKAVEEKRAL |
| | FGTIDSWLIWSLTGGVNGGVHCTDVTNASRTMLFNIHSLEWDKQLCEFFGIPMEILPN |
| | VRSSSEIYGLMKAGALEGVPISGCLGDQSAALVGQMCFQIGQAKNTYGTGCFLLCNTG |
| | HKCVFSDHGLLTTVAYKLGRDKPVYYALEGSVAIAGAVIRWLRDNLGIIKTSEEIEKL |
| | AKEVGTSYGCYFVPAFSGLYAPYWEPSARGIICGLTQFTNKCHIAFAALEAVCFQTRE |
| | ILDAMNRDCGIPLSHLQVDGGMTSNKILMQLQADILYIPVVKPSMPETTALGAAMAAG |
| | AAEGVGVWSLEPEDLSAVTMERFEPQINAEESEIRYSTWKKAVMKSMGWVTTQSPESG |
| | IP |

Sequence comparison of the above protein sequences yields the following sequence relationships shown in Table 44B.

TABLE 44B
Comparison of NOV44a against NOV44b through NOV44d.

| | | Identities/ |
|---|---|---|
| | | Similarities for |
| Protein | NOV44a Residues/ | the Matched |
| Sequence | Match Residues | Region |
| NOV44b | 1 . . . 552 | 524/558 (93%) |
| | 4 . . . 533 | 524/558 (93%) |
| NOV44c | 1 . . . 552 | 524/552 (94%) |
| | 1 . . . 524 | 524/552 (94%) |
| NOV44d | 1 . . . 552 | 523/552 (94%) |
| | 1 . . . 524 | 523/552 (94%) |

Further analysis of the NOV44a protein yielded the following properties shown in Table 44C.

TABLE 44C
Protein Sequence Properties NOV44a

| | |
|---|---|
| PSort | 0.4500 probability located in cytoplasm; 0.3731 probability |
| analysis: | located in microbody (peroxisome); 0.1000 probability |
| | located in mitochondrial matrix space; 0.1000 probability |
| | located in lysosome (lumen) |
| SignalP | No Known Signal Sequence Predicted |
| analysis: | |

A search of the NOV44a protein against the Geneseq database, a proprietary database that contains sequences published in patents and patent publication, yielded several homologous proteins shown in Table 44D.

TABLE 44D
Geneseq Results for NOV44a

| | | | Identities/ | |
|---|---|---|---|---|
| | | | Similarities for | |
| Geneseq | Protein/Organism/Length | NOV44a Residues/ | the Matched | Expect |
| Identifier | [Patent #, Date] | Match Residues | Region | Value |
| ABB66928 | Drosophila melanogaster | 10 . . . 548 | 277/542 (51%) | e−155 |
| | polypeptide SEQ ID NO 27576 - | 17 . . . 529 | 362/542 (66%) | |
| | Drosophila melanogaster, 538 aa. | | | |
| | [WO200171042-A2, 27 SEP. 2001] | | | |
| AAU60271 | Propionibacterium acnes | 15 . . . 542 | 266/530 (50%) | e−144 |
| | immunogenic protein #21167 - | 28 . . . 520 | 348/530 (65%) | |
| | Propionibacterium acnes, 526 aa. | | | |
| | [WO200181581-A2, 01 NOV. 2001] | | | |
| ABB57950 | Drosophila melanogaster | 12 . . . 545 | 251/538 (46%) | e−143 |
| | polypeptide SEQ ID NO 642 - | 32 . . . 537 | 356/538 (65%) | |
| | Drosophila melanogaster, 576 aa. | | | |
| | [WO200171042-A2, 27 SEP. 2001] | | | |
| ABB57948 | Drosophila melanogaster | 12 . . . 545 | 251/538 (46%) | e−143 |
| | polypeptide SEQ ID NO 636 - | 34 . . . 539 | 356/538 (65%) | |
| | Drosophila melanogaster, 578 aa. | | | |
| | [WO200171042-A2, 27 SEP. 2001] | | | |
| ABB57846 | Drosophila melanogaster | 12 . . . 545 | 251/538 (46%) | e−143 |
| | polypeptide SEQ ID NO 330 - | 32 . . . 537 | 356/538 (65%) | |
| | Drosophila melanogaster, 576 aa. | | | |
| | [WO200171042-A2, 27 SEP. 2001] | | | |

In a BLAST search of public sequence datbases, the NOV44a protein was found to have homology to the proteins shown in the BLASTP data in Table 44E.

TABLE 44E
Public BLASTP Results for NOV44a

| | | | Identities/ | |
|---|---|---|---|---|
| Protein | | | Similarities for | |
| Accession | | NOV44a Residues/ | the Matched | Expect |
| Number | Protein/Organism/Length | Match Residues | Portion | Value |
| P32189 | Glycerol kinase (EC 2.7.1.30) | 1 . . . 552 | 524/552 (94%) | 0.0 |
| | (ATP: glycerol 3-phosphotransferase) | 1 . . . 524 | 524/552 (94%) | |
| | (Glycerokinase) (GK) - Homo sapiens | | | |
| | (Human), 524 aa. | | | |
| Q14409 | Glycerol kinase, testis specific 1 (EC | 1 . . . 552 | 516/552 (93%) | 0.0 |
| | 2.7.1.30) (ATP: glycerol 3- | 1 . . . 524 | 518/552 (93%) | |
| | phosphotransferase) (Glycerokinase) | | | |
| | (GK) - Homo sapiens (Human), 553 | | | |
| | aa. | | | |
| Q64516 | Glycerol kinase (EC 2.7.1.30) | 1 . . . 552 | 510/552 (92%) | 0.0 |
| | (ATP: glycerol 3-phosphotransferase) | 1 . . . 524 | 521/552 (93%) | |
| | (Glycerokinase) (GK) - Mus musculus | | | |
| | (Mouse), 524 aa. | | | |
| Q63060 | Glycerol kinase (EC 2.7.1.30) | 1 . . . 552 | 510/552 (92%) | 0.0 |
| | (ATP: glycerol 3-phosphotransferase) | 1 . . . 524 | 519/552 (93%) | |
| | (Glycerokinase) (GK) (ATP- | | | |
| | stimulated glucocorticoid-receptor | | | |
| | translocation promoter) (ASTP) - | | | |
| | Rattus norvegicus (Rat), 524 aa. | | | |
| Q14410 | Glycerol kinase, testis specific 2 (EC | 1 . . . 552 | 461/552 (83%) | 0.0 |
| | 2.7.1.30) (ATP: glycerol 3- | 1 . . . 524 | 495/552 (89%) | |
| | phosphotransferase) (Glycerokinase) | | | |
| | (GK) - Homo sapiens (Human), 553 | | | |
| | aa. | | | |

PFam analysis predicts that the NOV44a protein contains the domains shown in the Table 44F.

TABLE 44F
Domain Analysis of NOV44a

| | | Identities/ | |
|---|---|---|---|
| | | Similarities | |
| | | for the Matched | Expect |
| Pfam Domain | NOV44a Match Region | Region | Value |
| FGGY | 12 . . . 294 | 99/293 (34%) | 2.9e−126 |
| | | 266/293 (91%) | |
| FGGY_C | 297 . . . 525 | 101/235 (43%) | 5.4e−110 |
| | | 222/235 (94%) | |

### Example 45

The NOV45 clone was analyzed, and the nucleotide and encoded polypeptide sequences are shown in Table 45A.

TABLE 45A
NOV45 Sequence Analysis

| | SEQ ID NO:365 | 1719 bp | |
|---|---|---|---|
| NOV45a, | GGCCGGACAGTCCGCCGAGGTGCTCGGTGGAGTCATGGCAGTGCCCTTTGTGGAAGAC | | |
| CG166282-01 | | | |
| DNA Sequence | TGGGACTTGGTGCAAACCCTGGGAGAAGGTGCCTATGGAGAAGTTCAACTTGCTGTGA | | |
| | ATAGAGTAACTGAAGAAGCAGTCGCAGTGAAGATTGTAGATATGAAGCGTGCCGTAGA | | |
| | CTGTCCAGAAAATATTAAGAAAGAGATCTGTATCAATAAAATGCTAAATCATGAAAAT | | |
| | GTAGTAAAATTCTATGGTCACAGGAGAGAAGGCAATATCCAATATTTATTTCTGGAGT | | |
| | ACTGTAGTGGAGGAGAGCTTTTTGACAGAATAGAGCCAGACATAGGCATGCCTGAACC | | |
| | AGATGCTCAGAGATTCTTCCATCAACTCATGGCAGGGGTGGTTTATCTGCATGGTATT | | |
| | GGAATAACTCACAGGGATATTAAACCAGAAAATCTTCTGTTGGATGAAAGGGATAACC | | |
| | TCAAAATCTCAGACTTTGGCTTGGCAACAGTATTTCGGTATAATAATCGTGAGCGTTT | | |
| | GTTGAACAAGATGTGTGGTACTTTACCATATGTTGCTCCAGAACTTCTGAAGAGAAGA | | |
| | GAATTTCATGCAGAACCAGTTGATGTTTGGTCCTGTGGAATAGTACTTACTGCAATGC | | |
| | TCGCTGGAGAATTGCCATGGGACCAACCCAGTGACAGCTGTCAGGAGTATTCTGACTG | | |
| | GAAAGAAAAAAAAACATACCTCAACCCTTGGAAAAAAATCGATTCTGCTCCTCTAGCT | | |
| | CTGCTGCATAAAATCTTAGTTGAGAATCCATCAGCAAGAATTACCATTCCAGACATCA | | |
| | AAAAAGATAGATGGTACAACAAACCCCTCAAGAAAGGGGCAAAAAGGCCCCGAGTCAC | | |
| | TTCAGGTGGTGTGTCAGAGTCTCCCAGTGGATTTTCTAAGCACATTCAATCCAATTTG | | |
| | GACTTCTCTCCAGTAAACAGTGCTTCTAGTGAAGAAAATGTGAAGTACTCCAGTTCTC | | |
| | AGCCAGAACCCCGCACAGGTCTTTCCTTATGGGATACCAGCCCCTCATACATTGATAA | | |
| | ATTGGTACAAGGGATCAGCTTTTCCCAGCCCACATGTCCTGATCATATGCTTTTGAAT | | |
| | AGTCAGTTACTTGGCACCCCAGGATCCTCACAGAACCCCTGGCAGCGGTTGGTCAAAA | | |
| | GAATGACACGATTTTTTACCAAATTGGATGCAGACAAATCTTATCAATGCCTGAAAGA | | |
| | GACTTGTGAGAAGTTGGGCTATCAATGGAAGAAAAGTTGTATGAATCAGGGTGATGGA | | |
| | TTGGAGTTCAAGAGACACTTCCTGAAGATTAAAGGGAAGCTGATTGATATTGTGAGCA | | |
| | GCCAGAAGGTTTGGCTTCCTGCCACATGATCGGACCATCGGCTCTGGGGAATCCTGGT | | |
| | GAATATAGTGCTGCTATGTTGACATTATTCTTCCTAGAGAAGATTATCCTGTCCTGCA | | |
| | AACTGCAAATAGTAGTTCCTGAAGTGTTCACTTCCCTGTTTATCCAAACATCTTCCAA | | |
| | TTTATTTTGTTTGTTCGGCATACAAATAATACCTATATCTTAATTGTAAGCAAAACTT | | |
| | TGGGGAAAGGATGAATAGAATTCATTTGATTATTTCTTCATGTGTGTTTAGTATCTGA | | |
| | ATTTGAAACTCATCTGGTGGAAACCAAGTTTCAGGGGACATGAGTTTTCCAGCTTTTA | | |
| | TACACACGTATCTCATTTTTATCAAAACATTTTGTTT | | |
| | ORF Start: ATG at 35 | | ORF Stop: TGA at 1361 |
| | SEQ ID NO:366 | 422 aa | MW at 50400.3 kD |

| | |
|---|---|
| NOV45a, | MAVPFVEDWDLVQTLGEGAYGEVQLAVNRVTEEAVAVKIVDMKRAVDCPENIKKEICI |
| CG166282-01 | |
| Protein Sequence | NKMLNHENVVKFYGHRREGNIQYLFLEYCSGGELFDRIEPDIGMPEPDAQRFFHQLMA |
| | CVVYLHGIGITHRDIKPENLLLDERDNLKISDFGLATVFRYNNRERLLNKMCGTLPYV |
| | APELLKRREFHAEPVDVWSCGIVLTAMLAGELPWDQPSDSCQEYSDWKEKKTYLNPWK |
| | KIDSAPLALLHKILVENPSARITIPDIKKDRWYNKPLKKGAKRPRVTSGGVSESPSGF |
| | SKHIQSNLDFSPVNSASSEENVKYSSSQPEPRTGLSLWDTSPSYIDKLVQGISFSQPT |
| | CPDHMLLNSQLLGTPGSSQNPWQRLVKRMTRFFTKLDADKSYQCLKETCEKLGYQWKK |
| | SCMNQGDGLEFKRHFLKIKGKLIDIVSSQKVWLPAT |

Further analysis of the NOV45a protein yielded the following properties shown in Table 45B.

TABLE 45B
Protein Sequence Properties NOV45a

| | |
|---|---|
| PSort | 0.3000 probability located in nucleus; 0.1000 probability |
| analysis: | located in mitochondrial matrix space; 0.1000 probability |
| | located in lysosome (lumen); 0.0423 probability located in |
| | microbody (peroxisome) |
| SignalP | No Known Signal Sequence Predicted |
| analysis: | |

A search of the NOV45a protein against the Geneseq database, a proprietary database that contains sequences published in patents and patent publication, yielded several homologous proteins shown in Table 45C.

TABLE 45C
Geneseq Results for NOV45a

| | | | Identi- | |
|---|---|---|---|---|
| | | | ties/Simi- | |
| | | NOV45a | larities | |
| | | Residues/ | for the | Ex- |
| Geneseq | Protein/Organism/Length | Match | Matched | pect |
| Identifier | [Patent #, Date] | Residues | Region | Value |
| AAU10752 | Human checkpoint protein | 1 . . . 442 | 442/476 | 0.0 |
| | chk1 - Homo sapiens, | | (92%) | |
| | 476 aa. | 1 . . . 476 | 442/476 | |
| | [US6307015-B1, | | (92%) | |
| | OCT. 23, 2001] | | | |
| AAE00662 | Human cell cycle check- | 1 . . . 442 | 442/476 | 0.0 |
| | point protein, hchk1, | | (92%) | |
| | alternative version #1 - | 1 . . . 476 | 442/476 | |
| | Homo sapiens, 476 aa. | | (92%) | |
| | [US6218109-B1, | | | |
| | APR. 17, 2001] | | | |
| AAG68374 | Human Chk1 kinase protein | 1 . . . 442 | 442/476 | 0.0 |
| | sequence - Homo sapiens, | | (92%) | |
| | 476 aa. | 1 . . . 476 | 442/476 | |
| | [WO200121771-A2, | | (92%) | |
| | MAR. 29, 2001] | | | |
| AAE01155 | Human Chk1 protein - | 1 . . . 442 | 442/476 | 0.0 |
| | Homo sapiens, 476 aa. | | (92%) | |
| | [US6211164-B1, | 1 . . . 476 | 442/476 | |
| | APR. 03, 2001] | | (92%) | |
| AAY54452 | A human checkpoint kinase | 1 . . . 442 | 442/476 | 0.0 |
| | (hChk1) polypeptide - | | (92%) | |
| | Homo sapiens, 476 aa. | 1 . . . 476 | 442/476 | |
| | [WO200003005-A2, | | (92%) | |
| | JAN. 20, 2000] | | | |

In a BLAST search of public sequence datbases, the NOV45a protein was found to have homology to the proteins shown in the BLASTP data in Table 45D.

TABLE 45D
Public BLASTP Results for NOV45a

| | | | Identi- | |
|---|---|---|---|---|
| | | | ties/Simi- | |
| | | NOV45a | larities | |
| Protein | | Residues/ | for the | Ex- |
| Accession | | Match | Matched | pect |
| Number | Protein/Organism/Length | Residues | Portion | Value |
| O14757 | Serine/threonine-protein | 1 . . . 442 | 442/476 | 0.0 |
| | kinase Chk1 (EC 2.7.1.-) - | | (92%) | |
| | Homo sapiens (Human), | 1 . . . 476 | 442/476 | |
| | 476 aa. | | (92%) | |
| Q91ZN7 | Checkpoint kinase 1 (Cell | 1 . . . 442 | 420/476 | 0.0 |
| | cycle checkpoint protein | | (88%) | |
| | kinase) - Rattus norvegicus | 1 . . . 476 | 430/476 | |
| | (Rat), 476 aa. | | (90%) | |
| Q9D0N2 | Checkpoint kinase 1 homo- | 1 . . . 442 | 414/476 | 0.0 |
| | log (S. pombe) - Mus | | (86%) | |
| | musculus (Mouse), 476 aa. | 1 . . . 476 | 428/476 | |
| | | | (88%) | |
| O35280 | Serine/threonine-protein | 1 . . . 442 | 411/476 | 0.0 |
| | kinase Chk1 (EC 2.7.1.-) - | | (86%) | |
| | Mus musculus (Mouse), | 1 . . . 476 | 427/476 | |
| | 476 aa. | | (89%) | |
| AAN33019 | Checkpoint 1 protein - | 1 . . . 440 | 371/474 | 0.0 |
| | Gallus gallus (Chicken), | | (78%) | |
| | 476 aa. | 1 . . . 474 | 403/474 | |
| | | | (84%) | |

PFam analysis predicts that the NOV45a protein contains the domains shown in the Table 45E.

TABLE 45E
Domain Analysis of NOV45a

| | | Identities/ | |
|---|---|---|---|
| | | Similarities | |
| | | for the Matched | Expect |
| Pfam Domain | NOV45a Match Region | Region | Value |
| pkinase | 9 . . . 265 | 93/294 (32%) | 1.2e−75 |
| | | 201/294 (68%) | |

### Example 46

The NOV46 clone was analyzed, and the nucleotide and encoded polypeptide sequences are shown in Table 46A.

TABLE 46A
NOV46 Sequence Analysis

| | SEQ ID NO:367 | 2264 bp | |
|---|---|---|---|
| NOV46a, | TTGACTGTATCGCCGGAATTCATGGCAGCGCCAGGCGGCAGGTCGGAGCCGCCGCAGC | | |
| CG170739-01 | | | |
| DNA Sequence | TCCCCGAGTACAGCTGCAGCTACATGGTGTCGCGGCCGGTCTACAGCGAGCTCGCTTT | | |
| | CCAGCAACAGCACGAGCGGCGCCTGCAGGAGCGCAAGACGCTGCGGGAGAGCCTGGCC | | |
| | AAGTGCTGCAGTTGTTCAAGAAAGAGAGCCTTTGGTGTGCTAAAGACTCTAGTGCCCA | | |
| | TCTTGGAGTGGCTCCCCAAATACCGAGTCAAGGAATCGCTGCTTAGTGACGTCATTTC | | |
| | GGGAGTTAGTACTGGGCTAGTGGCCACGCTGCAAGGACCTTTTCCAGTGGTGAGTTTA | | |
| | ATGGTGGGATCTGTTGTTCTGAGCATGGCCCCCGACGAACACTTTCTCGTATCCAGCA | | |
| | GCAATGGAACTGTATTAAATACTACTATGATAGACACTGCAGCTAGAGATACAGCCAG | | |
| | AGTCCTGATTGCCAGTGCCCTGACTCTGCTGGTTGGAATTATACAGTTGATATTTGGT | | |
| | GGCTTGCAGATTGGATTCATAGTGAGGCACTTGGCAGATCCTTTGGTTGGTGGCTTCA | | |
| | CAACAGCTGCTGCCTTCCAAGTGCTGGTCTCACAGCTAAAGATTGTCCTCAATGTTTC | | |
| | AACCAAAAACTACAATGGAGTTCTCTCTATTATCTATACGCTGGTTGAGATTTTTCAA | | |
| | AATATTGGTGATACCAATCTTGCTGATTTCACTGCTGGATTGCTCACCATTGTCGTCT | | |
| | GTATGGCAGTTAAGGAATTAAATGATCGGTTTAGACACAAAATCCCAGTCCCTATTCC | | |
| | TATAGAAGTAATTGTGACGATAATTGCTACTGCCATTTCATATGGAGCCAACCTGGAA | | |
| | AAAAATTACAATGCTGGCATTGTTAAATCCATCCCAAGGGGGTTTTTGCCTCCTGAAC | | |
| | TTCCACCTGTGAGCTTGTTCTCGGAGATGCTGGCTGCATCATTTTCCATCGCTGTGGT | | |
| | GGCTTATGCTATTGCAGTGTCAGTAGGAAAAGTATATGCCACCAAGTATGATTACACC | | |
| | ATCGATGGGAACCAGGAATTCATTGCCTTTGGGATCAGCAACATCTTCTCAGGATTCT | | |
| | TCTCTTGTTTTGTGGCCACCACTGCTCTTTCCCGCACGGCCGTCCAGGAGAGCACTGG | | |
| | AGGAAAGACACAGGTTGCTGGCATCATCTCTCCTGCGATTGTGATGATCGCCATTCTT | | |
| | GCCCTGGGGAAGCTTCTGGAACCCTTGCAGAAGTCGGTCTTGGCAGCTGTTGTAATTG | | |
| | CCAACCTGAAAGGGATGTTTATGCAGCTGTGTGACATTCCTCGTCTGTGGAGACAGAA | | |
| | TAAGATTGATGCTGTTATCTGGGTGTTTACGTGTATAGTGTCCATCATTCTGGGGCTG | | |
| | GATCTCGGTTTACTAGCTGGCCTTATATTTGGACTGTTGACTGTGGTCCTGAGAGTTC | | |
| | AGTTTCCTTCTTGGAATGGCCTTGGAAGCATCCCTAGCACAGATATCTACAAAAGTAC | | |
| | CAAGAATTACAAAAACATTGAAGAACCTCAAGGAGTGAAGATTCTTAGATTTTCCAGT | | |
| | CCTATTTTCTATGGCAATGTCGATGGTTTTAAAAAATGTATCAAGTCCACAGTTGGAT | | |
| | TTGATGCCATTAGAGTATATAATAAGAGGCTGAAAGCGCTGAGGAAAATACAGAAACT | | |
| | AATAAAAAGTGGACAATTAAGAGCAACGAAGAATGGCATCATAAGTGATGCTGTTTCA | | |
| | ACAAATAATGCTTTTGAGCCCGATGAGGATATTGAAGATCTGGAGGAACTTGATATCC | | |
| | CAACCAAGGAAATAGAGATTCAAGTGGATTGGAACTCTGAGCTTCCAGTCAAAGTGAA | | |
| | CGTTCCCAAAGTGCCAATCCATAGCCTTGTGCTTGACTGTGGAGCTATATCTTTCCTG | | |
| | GACGTTGTTGGAGTGAGATCACTGCGGGTGATTGTCAAAGAATTCCAAAGAATTGATG | | |
| | TGAATGTGTATTTTGCATCACTTCAAGATTATGTGATAGAAAAGCTGGAGCAATGCGG | | |
| | GTTCTTTGACGACAACATTAGAAAGGACACATTCTTTTTGACGGTCCATGATGCTATA | | |
| | CTCTATCTACAGAACCAAGTGAAATCTCAAGAGGGTCAAGGTTCCATTTTAGAAACGA | | |
| | TCACTCTCATTCAGGATTGTAAAGATACCCTTGAATTAGTAGAAACAGAGCTGACGGA | | |
| | AGAAGAACTTGATGTCCAGGATGAGGCTATGCGTACACTTGCATCCTGACTGCAGCCA | | |
| | ORF Start: ATG at 22 | | ORF Stop: TGA at 2251 |
| | SEQ ID NO:368 | 743 aa | MW at 81685.2 kD |

| | |
|---|---|
| NOV46a, | MAAPGGRSEPPQLPEYSCSYMVSRPVYSELAFQQQHERRLQERKTLRESLAKCCSCSR |
| CG170739-01 | |
| Protein Sequence | KRAFGVLKTLVPILEWLPKYRVKEWLLSDVISGVSTGLVATLQGPFPVVSLMVGSVVL |
| | SMAPDEHFLVSSSNGTVLNTTMIDTAARDTARVLIASALTLLVGIIQLIFGGLQIGFI |
| | VRHLADPLVGGFTTAAAFQVLVSQLKIVLNVSTKNYNGVLSIIYTLVEIFQNIGDTNL |
| | ADFTAGLLTIVVCMAVKELNDRFRHKIPVPIPIEVIVTIIATAISYGANLEKNYNAGI |
| | VKSIPRGFLPPELPPVSLFSEMLAASPSIAVVAYAIAVSVGKVYATKYDYTIDGNQEF |
| | IAFGISNIFSGFFSCFVATTALSRTAVQESTGGKTQVAGIISAAIVMIAILALGKLLE |
| | PLQKSVLAAVVIANLKGMFMQLCDIPRLWRQNKIDAVIWVFTCIVSIILGLDLGLLAG |
| | LIFGLLTVVLRVQFPSWNGLGSIPSTDIYKSTKNYKNIEEPQGVKILRFSSPIFYGNV |
| | DGFKKCIKSTVGFDAIRVYNKRLKALRKIQKLIKSGQLRATKNGIISDAVSTNNAFEP |
| | DEDIEDLEELDIPTKEIEIQVDWNSELPVKVNVPKVPIHSLVLDCGAISFLDVVGVRS |
| | LRVIVKEFQRIDVNVYFASLQDYVIEKLEQCGFFDDNIRKDTFFLTVHDAILYLQNQV |
| | KSQEGQGSILETITLIQDCKDTLELVETELTEEELDVQDEAMRTLAS |

Further analysis of the NOV46a protein yielded the following properties shown in Table 46B.

TABLE 46B
Protein Sequence Properties NOV46a

| | |
|---|---|
| PSort | 0.8000 probability located in plasma membrane; 0.4000 prob- |
| analysis: | ability located in Golgi body; 0.3000 probability located in |
| | endoplasmic reticulum (membrane); 0.0300 probability lo- |
| | cated in mitochondrial inner membrane |
| SignalP | No Known Signal Sequence Predicted |
| analysis: | |

A search of the NOV46a protein against the Geneseq database, a proprietary database that contains sequences published in patents and patent publication, yielded several homologous proteins shown in Table 46C.

TABLE 46C
Geneseq Results for NOV46a

| | | NOV46a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| ABG61914 | Prostate cancer-associated protein | 1 . . . 743 | 741/780 (95%) | 0.0 |
| | #115 - Mammalia, 790 aa. | 1 . . . 780 | 743/780 (95%) | |
| | [WO200230268-A2, 18 APR. 2002] | | | |
| AAM51696 | Human pendrin SEQ ID NO 2 - | 1 . . . 743 | 741/780 (95%) | 0.0 |
| | Homo sapiens, 780 aa. | 1 . . . 780 | 743/780 (95%) | |
| | [JP2001228146-A, 24 AUG. 2001] | | | |
| AAM51695 | Mouse pendrin SEQ ID NO 1 - Mus | 1 . . . 743 | 648/780 (83%) | 0.0 |
| | sp, 780 aa. [JP2001228146-A, 24 | 1 . . . 780 | 701/780 (89%) | |
| | AUG. 2001] | | | |
| AAR60568 | Down-regulated in adenoma DRA | 20 . . . 692 | 322/716 (44%) | e−176 |
| | tumor suppressor - Homo sapiens, | 9 . . . 720 | 448/716 (61%) | |
| | 764 aa. [WO9420616-A, 15 SEP. | | | |
| | 1994] | | | |
| AAG67162 | Amino acid sequence of a human | 56 . . . 691 | 257/689 (37%) | e−132 |
| | 32613 transporter polypeptide - | 62 . . . 733 | 401/689 (57%) | |
| | Homo sapiens, 751 aa. | | | |
| | [WO200164875-A2, 07 SEP. 2001] | | | |

In a BLAST search of public sequence datbases, the NOV46a protein was found to have homology to the proteins shown in the BLASTP data in Table 46D.

TABLE 46D
Public BLASTP Results for NOV46a

| | | NOV46a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O43511 | Pendrin (Sodium-independent | 1 . . . 743 | 741/780 (95%) | 0.0 |
| | chloride/iodide transporter) - Homo | 1 . . . 780 | 743/780 (95%) | |
| | sapiens (Human), 780 aa. | | | |
| Q9R154 | Pendrin (Sodium-independent | 1 . . . 743 | 656/780 (84%) | 0.0 |
| | chloride/iodide transporter) - Rattus | 1 . . . 780 | 700/780 (89%) | |
| | norvegicus (Rat), 780 aa. | | | |
| Q9R155 | Pendrin (Sodium-independent | 1 . . . 743 | 648/780 (83%) | 0.0 |
| | chloride/iodide transporter) - Mus | 1 . . . 780 | 701/780 (89%) | |
| | musculus (Mouse), 780 aa. | | | |
| Q924C9 | Chloride anion exchanger (DRA | 20 . . . 692 | 330/715 (46%) | 0.0 |
| | protein) (Down-regulated in | 9 . . . 713 | 470/715 (65%) | |
| | adenoma) - Rattus norvegicus | | | |
| | (Rat), 757 aa. | | | |
| Q9WVC8 | Chloride anion exchanger (DRA | 20 . . . 692 | 328/715 (45%) | 0.0 |
| | protein) (Down-regulated in | 9 . . . 713 | 463/715 (63%) | |
| | adenoma) - Mus musculus (Mouse), | | | |
| | 757 aa. | | | |

PFam analysis predicts that the NOV46a protein contains the domains shown in the Table 46E.

TABLE 46E
Domain Analysis of NOV46a

| | | Identities/ | |
|---|---|---|---|
| | NOV46a | Similarities | |
| | Match | for the | Expect |
| Pfam Domain | Region | Matched Region | Value |
| 7tm_3 | 171 . . . 410 | 48/293 (16%) | 0.46 |
| | | 137/293 (47%) | |
| Xan_ur_permease | 85 . . . 465 | 67/468 (14%) | 0.56 |
| | | 234/468 (50%) | |
| Sulfate_transp | 166 . . . 476 | 110/328 (34%) | 1.8e−97 |
| | | 265/328 (81%) | |
| STAS | 499 . . . 688 | 32/192 (17%) | 1.6e−30 |
| | | 147/192 (77%) | |

### Example 47

The NOV47 clone was analyzed, and the nucleotide and encoded polypeptide sequences are shown in Table 47A.

TABLE 47A
NOV47 Sequence Analysis

| | SEQ ID NO:369 | 1337 bp | |
|---|---|---|---|
| NOV47a, | ATGAGATTTGGCATCTTTCTTTTGTGGTGGGGATGGGTTTTGGCCACTGAAAGCAGAA | | |
| CG171632-01 | | | |
| DNA Sequence | TGCGCTGGCCCGGAAGAGAAGTCCACGAGATGTCTAAGAAAGGCAGTAGGCCCCAAAG | | |
| | ACAAACACGAGAAGTACATGAAGATGCCCACAAGCAAGTCAGCCCAATTCTGAGACGA | | |
| | AGTCCTGCCATTCCTGTTGGTGTGGATGTGCAGGTGGAGAGTTTGCATAGCATCTCAG | | |
| | AGGTTGACATGGACTTTACGATGACCCTCTACCTGAGGCACTACTGGAAGGACGAGAG | | |
| | GCTGTCTTTTCCAAGCACCAACAACCTCAGCATGACGTTTGATGGCCGGCTGGTCAAG | | |
| | AAGATCTGGGTCCCTGACATGTTTTTCGTGCACTCCAAACGCTCCTTCATCCACGACA | | |
| | CCACCACAGACAACGTCATGTTGCGGGTCCAGCCTGATGGGAAAGTGCTCTATAGTCT | | |
| | CAGGGTTACAGTAACTGCAATGTGCAACATGGACTTCAGCCGATTTCCCTTGGACACA | | |
| | CAAACGTGCTCTCTTGAAATTGAAAGCTATGCCTATACAGAAGATGACCTCATGCTGT | | |
| | ACTGGAAAAAGGGCAATGACTCCTTAAAGACAGATGAACGGATCTCACTCTCCCAGTT | | |
| | CCTCATTCAGGAATTCCACACCACCACCAAACTGGCTTTCTACAGCAGCACAGGCTGG | | |
| | TACAACCGTCTCTACATTAATTTCACGTTGCGTCGCCACATCTTCTTCTTCTTGCCCC | | |
| | AAACTTATTTCCCCGCTACCCTGGTGGTCATGCTGTCCTGGGTGTCCTTCTGGATCGA | | |
| | CCGCAGAGCCGTGCCTGCCAGAGTCCCCTTAGGTATCACAACGCTCCTGACCATGTCC | | |
| | ACCATCATCACGGGCGTGAATGCCTCCATGCCGCGCGTCTCCTACATCAAGGCCGTGG | | |
| | ACATCTACCTCTGGGTCAGCTTTGTGTTCGTGTTCCTCTCGGTGCTGGAGTATGCGGC | | |
| | CGTCAACTACCTGACCACTGTGCAGGAGAGGAAGGAACAGAAGCTGCGGGAGAAGCTT | | |
| | CCCTGCACCAGCGGATTACCTCCGCCCAACACTGCGATGCTGGACGGCAACTACAGTC | | |
| | ATGGGGAGGTGAATGACCTGGACAACTACATGCCAGAGAATGGAGAGAAGCCCGACAG | | |
| | GATGATGGTGCAGCTGACCCTGGCCTCAGAGAGGAGCTCCCCACAGAGGAAAAGTCAG | | |
| | AGAAGCAGCTATGTGAGCATGAGAATCGACACCCACGCCATTGATAAATACTCCAGGA | | |
| | TCATCTTTCCAGCAGCATACATTTTATTCAATTTAATATACTGGTCTATTTTCTCCTA | | |
| | GAT | | |
| | ORF Start: ATG at 1 | | ORF Stop: TAG at 1333 |
| | SEQ ID NO:370 | 444 aa | MW at 51932.2 kD |
| NOV47a, | MRFGIFLLWWGWVLATESRMRWPGREVHEMSKKGSRPQRQRREVHEDAHKQVSPILRR | | |
| CG171632-01 | | | |
| Protein Sequence | SPAIPVGVDVQVESLDSISEVDMDFTMTLYLRHYWKDERLSFPSTNNLSMTFDGRLVK | | |
| | KIWVPDMFFVHSKRSFIHDTTTDNVMLRVQPDGKVLYSLRVTVTAMCNMDFSRFPLDT | | |
| | QTCSLEIESYAYTEDDLMLYWKKGNDSLKTDERISLSQFLIQEFHTTTKLAFYSSTGW | | |
| | YNRLYINFTLRRHIFFFLPQTYFPATLVVMLSWVSFWIDRRAVPARVPLGITTVLTMS | | |
| | TIITGVNASMPRVSYIKAVDIYLWVSFVFVFLSVLEYAAVNYLTTVQERKEQKLREKL | | |
| | PCTSGLPPPNTAMLDGNYSDGEVNDLDNYMPENGEKPDRMMVQLTLASERSSPQRKSQ | | |
| | RSSYVSMRIDTHAIDKYSRTIFPAAYILFNLIYWSIFS | | |
| | SEQ ID NO:371 | 1337 bp | |
| NOV47b, | ATGAGATTTGGCATCTTTCTTTTGTGGTGGGGATGGGTTTTGGCCACTGAAAGCAGAA | | |
| CG171632-01 | | | |
| DNA Sequence | TGCGCTGGCCCGGAAGAGAAGTCCACGAGATGTCTAAGAAAGGCAGTAGGCCCCAAAG | | |
| | ACAAAGACGAGAAGTACATGAAGATGCCCACAAGCAAGTCAGCCCAATTCTGAGACGA | | |
| | AGTCCTGCCATTCCTGTTGGTGTGGATGTGCAGGTAAAGAGTTTGGATAGCATCTCAG | | |
| | AGGTTGACATGGACTTTACGATGACCCTCTACCTGACCCACTACTGGAAGGACGAGAG | | |
| | GCTGTCTTTTCCAAGCACCAACAACCTCAGCATGACGTTTGATGGCCGGCTGGTCAAG | | |
| | AAGATCTGGGTCCCTGACATGTTTTTCGTGCACTCCAAACGCTCCTTCATCCACGACA | | |
| | CCACCACAGACAACGTCATGTTGCGGGTCCAGCCTGATGGGAAAGTGCTCTATAGTCT | | |
| | CAGGGTTACAGTAACTGCAATGTGCAACATGGACTTCAGCCGATTTCCCTTGGACACA | | |
| | CAAACGTGCTCTCTTGAAATTGAAAGCTATGCCTATACAGAAGATGACCTCATGCTGT | | |
| | ACTGGAAAAAGGGCAATGACTCCTTAAAGACAGATGAACGGATCTCACTCTCCCAGTT | | |
| | CCTCATTCAGGAATTCCACACCACCACCAAACTGGCTTTCTACAGCAGCACAGGCTGG | | |
| | TACAACCGTCTCTACATTAATTTCACGTTGCGTCGCCACATCTTCTTCTTCTTGCCCC | | |
| | AAACTTATTTCCCCGCTACCCTGGTGGTCATGCTGTCCTGGGTGTCCTTCTGGATCGA | | |
| | CCGCAGAGCCGTGCCTGCCAGAGTCCCCTTAGGTATCACAACGGTGCTGACCATGTCC | | |
| | ACCATCATCACGGGCGTGAATGCCTCCATGCCGCGCGTCTCCTACATCAAGGCCGTGG | | |
| | ACATCTACCTCTGGGTCAGCTTTGTGTTCGTGTTCCTCTCGGTGCTGGAGTATGCGGC | | |
| | CGTCAACTACCTGACCACTGTGCAGGAGAGGAAGGAACAGAAGCTGCGGGAGAAGCTT | | |
| | CCCTGCACCAGCGGATTACCTCCGCCCAACACTGCGATGCTGGACGGCAACTACAGTG | | |
| | ATGGGGAGGTGAATGACCTGGACAACTACATGCCAGAGAATGGACAGAAGCCCGACAG | | |
| | GATGATGGTGCAGCTGACCCTGGCCTCAGAGAGGAGCTCCCCACAGAGGAAAAGTCAG | | |
| | AGAAGCAGCTATGTGAGCATGAGAATCGACACCCACGCCATTGATAAATACTCCAGGA | | |
| | TCATCTTTCCAGCAGCATACATTTTATTCAATTTAATATACTGGTCTATTTTCTCCTA | | |
| | GAT | | |
| | ORF Start: ATG at 1 | | ORF Stop: TAG at 1333 |
| | SEQ ID NO:372 | 444 aa | MW at 51932.2 kD |

| | |
|---|---|
| NOV47b, | MRFGIFLLWWGWVLATESRMRWPGREVHEMSKKGSRPQRQRREVHEDAHKQVSPTLRR |
| CG171632-01 | |
| Protein Sequence | SPAIPVGVDVQVESLDSISEVDMDFTMTLYLRHYWKDERLSFPSTNNLSMTFDGRLVK |
| | KINVPDMFFVHSKRSFIHDTTTDNVMLRVQPDGKVLYSLRVTVTAMCNMDFSRFPLDT |
| | QTCSLEIESYAYTEDDLMLYWKKGNDSLKTDERISLSQFLIQEFHTTTKLAFYSSTGW |
| | YNRLYINFTLRRHIFFFLPQTYFPATLVVMLSWVSFWIDRRAVPARVPLCITTVLTMS |
| | TIITGVNASMPRVSYIKAVDIYLWVSFVFVFLSVLEYAAVNYLTTVQERKEQKLREKL |
| | PCTSGLPPPNTANLDGNYSDGEVNDLDNYMPENGEKPDRMMVQLTLASERSSPQRKSQ |
| | RSSYVSMRIDTHAIDKYSRIIFPAAYTLFNLIYWSIFS |

Sequence comparison of the above protein sequences yields the following sequence relationships shown in Table 47B.

TABLE 47B
Comparison of NOV47a against NOV47b.

| | NOV47a Residues/ | Identities/Similarities |
|---|---|---|
| Protein Sequence | Match Residues | for the Matched Region |
| NOV47b | 1 . . . 444 | 444/444 (100%) |
| | 1 . . . 444 | 444/444 (100%) |

Further analysis of the NOV47a protein yielded the following properties shown in Table 47C.

TABLE 47C
Protein Sequence Properties NOV47a

| | |
|---|---|
| PSort | 0.4600 probability located in plasma membrane; 0.1692 |
| analysis: | probability located inmicrobody (peroxisome); |
| | 0.1000 probability located in endoplasmic reticulum |
| | (membrane); 0.1000 probability located in |
| | endoplasmic reticulum (lumen) |
| SignalP | Cleavage site between residues 16 and 17 |
| analysis: | |

A search of the NOV47a protein against the Geneseq database, a proprietary database that contains sequences published in patents and patent publication, yielded several homologous proteins shown in Table 47D.

TABLE 47D
Geneseq Results for NOV47a

| | | NOV47a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAE21956 | Human transporter protein - Homo | 18 . . . 443 | 268/432 (62%) | e−149 |
| | sapiens, 467 aa. [US2002028773- | 36 . . . 466 | 320/432 (74%) | |
| | A1, 07 MAR. 2002] | | | |
| AAU04467 | Human gamma-amino butyric acid | 18 . . . 443 | 268/432 (62%) | e−149 |
| | (GABA) receptor protein #1 - Homo | 36 . . . 466 | 320/432 (74%) | |
| | sapiens, 467 aa. [WO200153489- | | | |
| | A1, 26 JUL. 2001] | | | |
| AAU04470 | Human gamma-amino butyric acid | 35 . . . 443 | 263/412 (63%) | e−149 |
| | (GABA) receptor protein #4 - Homo | 9 . . . 419 | 313/412 (75%) | |
| | sapiens, 420 aa. [WO200153489- | | | |
| | A1, 26 JUL. 2001] | | | |
| AAG68256 | Human POLY3 protein sequence | 18 . . . 443 | 266/433 (61%) | e−146 |
| | SEQ ID NO: 6 - Homo sapiens, 468 | 36 . . . 467 | 318/433 (73%) | |
| | aa. [WO200179294-A2, 25 OCT. | | | |
| | 2001] | | | |
| AAO14188 | Human transporter and ion channel | 18 . . . 443 | 264/432 (61%) | e−146 |
| | TRICH-5 - Homo sapiens, 467 aa. | 36 . . . 466 | 317/432 (73%) | |
| | [WO200204520-A2, 17 JAN. 2002] | | | |

In a BLAST search of public sequence datbases, the NOV47a protein was found to have homology to the proteins shown in the BLASTP data in Table 47E.

TABLE 47E
Public BLASTP Results for NOV47a

| | | NOV47a | Identities/ | |
|---|---|---|---|---|
| Protein | | Residues/ | Similarities | |
| Accession | | Match | for the | Expect |
| Number | Protein/Organism/Length | Residues | Matched Portion | Value |
| P24046 | Gamma-aminobutyric-acid receptor | 1 . . . 444 | 439/474 (92%) | 0.0 |
| | rho-1 subunit precursor (GABA(A) | 1 . . . 473 | 440/474 (92%) | |
| | receptor) - Homo sapiens (Human), | | | |
| | 473 aa. | | | |
| P50572 | Gamma-aminobutyric-acid receptor | 1 . . . 444 | 416/474 (87%) | 0.0 |
| | rho-1 subunit precursor (GABA(A) | 1 . . . 474 | 425/474 (88%) | |
| | receptor) - Rattus norvegicus (Rat), | | | |
| | 474 aa. | | | |
| P56475 | Gamma-aminobutyric-acid receptor | 1 . . . 444 | 413/474 (87%) | 0.0 |
| | rho-1 subunit precursor (GABA(A) | 1 . . . 474 | 423/474 (89%) | |
| | receptor) - Mus musculus (Mouse), | | | |
| | 474 aa. | | | |
| Q8UW04 | GABA receptor rho-1 subunit - | 23 . . . 443 | 325/427 (76%) | 0.0 |
| | Fugu rubripes (Japanese pufferfish) | 54 . . . 479 | 361/427 (84%) | |
| | (Takifugu rubripes), 480 aa. | | | |
| Q9YGQ4 | Gamma-aminobutyric-acid receptor | 60 . . . 444 | 317/389 (81%) | 0.0 |
| | rho-1A subunit - Morone americana | 89 . . . 476 | 345/389 (88%) | |
| | (White perch), 476 aa. | | | |

PFam analysis predicts that the NOV47a protein contains the domains shown in the Table 47F.

TABLE 47F
Domain Analysis of NOV47a

| | | Identities/ | |
|---|---|---|---|
| | | Similarities | |
| | NOV47a | for the | Expect |
| Pfam Domain | Match Region | Matched Region | Value |
| Neur_chan_LBD | 59 . . . 246 | 64/242 (26%) | 8.3e−71 |
| | | 168/242 (69%) | |
| Neur_chan_memb | 253 . . . 440 | 40/291 (14%) | 2.6e−52 |
| | | 154/291 (53%) | |

### Example 48

The NOV48 clone was analyzed, and the nucleotide and encoded polypeptide sequences are shown in Table 48A.

TABLE 48A
NOV48 Sequence Analysis

| | SEQ ID NO:373 | 1118 bp | |
|---|---|---|---|
| NOV48a, | GCCCTTAGATCAAGATGCGCTGTAACTGAGAAGCCCCCAAGGCGGAGGCTGAGAATCA | | |
| CG173066-01 | | | |
| DNA Sequence | GAGACATTTCAGCAGACATCTACAAATCCGAAAGACAAAACATGGTTCAAGCATCCGG | | |
| | GCACAGGCGGTCCACCCGTGGCTCCAAAATGGTCTCCTGGTCCGTGATAGCAAAGATC | | |
| | CAGGAAATACTGCAGAGGAAGATGGTGCGAGAGTTCCTGGCCGAGTTCATGAGCACAT | | |
| | ATGTCATGATGGTATTCGGCCTTGGTTCCGTGGCCCATATGGTTCTAAATAAAAAATA | | |
| | TGGGAGCTACCTTGGTGTCAACTTGGGTTTTGGCTTCGGAGTCACCATGGGAGTGCAC | | |
| | GTGGCAGGCCGCATCTCTGGAGCCCACATGAACGCAGCTGTGACCTTTGCTAACTGTG | | |
| | CGCTGGGCCGCGTGCCCTGGAGGAAGTTTCCGGTCTATGTGCTGGGGCAGTTCCTGGG | | |
| | CTCCTTCCTGGCGGCTGCCACCATCTACAGTCTCTTCTACACGGCCATTCTCCACTTT | | |
| | TCGGGTGGACAGCTGATGGTGACCGGTCCCGTCGCTACAGCTGGCATTTTTGCCACCT | | |
| | ACCTTCCTGATCACATGACATTGTGGCGGGGCTTCCTGAATGAGGCGTGGCTGACCGG | | |
| | GATGCTCCAGCTGTGCCTCTTCGCCATCACGGACCAGGAGAACAACCCAGCACTGCCA | | |
| | GGAACAGAGGCGCTGGTGATAGGCATCCTCGTGGTCATCATCGGGGTGTCCCTTGGCA | | |
| | TGAACACAGGATATGCCATCAACCCGTCCCGGGACCTGCCCCCCCGCATCTTCACCTT | | |
| | CATTGCTGGTTGGGGCAAACAGGTCTTCAGGTGGCATCATCTACCTGGTCTTCATTGG | | |
| | CTCCACCATCCCACGGGAGCCCCTGAAATTGGAGGATTCTGTGGCGTATGAAGACCAC | | |
| | GGGATAACCGTATTGCCCAAGATGGGATCTCATGAACCCACGATCTCTCCCCTCACCC | | |
| | CCGTCTCTGTGAGCCCTGCCAACAGATCTTCAGTCCACCCTGCCCCACCCTTACATGA | | |
| | ATCCATAGCCCTAGAGCACTTCTAAGCAGAGATTATTTGTGATCCCATCCATTCCCCA | | |
| | ATAAAGCAAGGCTTGT | | |
| | ORF Start: ATG at 100 | | ORF Stop: TGA at 919 |
| | SEQ ID NO:374 | 273 aa | MW at 29820.8 kD |

| | |
|---|---|
| NOV48a, | MVQASGHRRSTRGSKMVSWSVIAKIQEILQRKMVREFLAEFMSTYVMMVFGLGSVAHM |
| CG173066-01 | |
| Protein Sequence | VLNKKYGSYLGVNLGFGFGVTMGVHVAGRISGAHMNAAVTFANCALGRVPWRKFPVYV |
| | LGQFLGSFLAAATIYSLFYTAILHFSGGQLMVTGPVATAGIFATYLPDHMTLWRGFLN |
| | EAWLTGMLQLCLFAITDQENNPALPGTEALVIGILVVIIGVSLGMNTGYAINPSRDLP |
| | PRIFTFIAGWGKQVFRWHHLPGLHWLHHPTGAPEIGGFCGV |

Further analysis of the NOV48a protein yielded the following properties shown in Table 48B.

TABLE 48B
Protein Sequence Properties NOV48a

| | |
|---|---|
| PSort | 0.8586 probability located in mitochondrial |
| analysis: | inner membrane; 0.7000 probability located in |
| | plasma membrane; 0.6400 probability located in |
| | microbody (peroxisome); 0.3568 probability |
| | located in mitochondrial intermembrane space |
| SignalP | No Known Signal Sequence Predicted |
| analysis: | |

A search of the NOV48a protein against the Geneseq database, a proprietary database that contains sequences published in patents and patent publication, yielded several homologous proteins shown in Table 48C.

TABLE 48C
Geneseq Results for NOV48a

| | | NOV48a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAW87644 | A protein with water channel | 1 . . . 272 | 253/272 (93%) | e−143 |
| | activity - Homo sapiens, 342 aa. | 1 . . . 268 | 256/272 (94%) | |
| | [WO9843997-A1, 08 OCT. 1998] | | | |
| AAY70455 | Human membrane channel protein- | 5 . . . 272 | 249/269 (92%) | e−140 |
| | 5 (MECHP-5) - Homo sapiens, 341 | 3 . . . 267 | 252/269 (93%) | |
| | aa. [WO200012711-A2, 09 MAR. | | | |
| | 2000] | | | |
| AAE13275 | Human transporters and ion | 1 . . . 272 | 236/276 (85%) | e−130 |
| | channels (TRICH)-2 - Homo | 1 . . . 272 | 243/276 (87%) | |
| | sapiens, 346 aa. [WO200177174- | | | |
| | A2, 18 OCT. 2001] | | | |
| ABG27139 | Novel human diagnostic protein | 49 . . . 273 | 217/225 (96%) | e−130 |
| | #27130 - Homo sapiens, 225 aa. | 1 . . . 225 | 221/225 (97%) | |
| | [WO200175067-A2, 11 OCT. 2001] | | | |
| ABB57440 | Human secreted protein encoding | 29 . . . 273 | 116/246 (47%) | 3e−64 |
| | polypeptide SEQ ID NO 86 - Homo | 17 . . . 258 | 165/246 (66%) | |
| | sapiens, 292 aa. [WO200183510- | | | |
| | A1, 08 NOV. 2001] | | | |

In a BLAST search of public sequence datbases, the NOV48a protein was found to have homology to the proteins shown in the BLASTP data in Table 48D.

TABLE 48D
Public BLASTP Results for NOV48a

| | | NOV48a | Identities/ | |
|---|---|---|---|---|
| Protein | | Residues/ | Similarities | |
| Accession | | Match | for the | Expect |
| Number | Protein/Organism/Length | Residues | Matched Portion | Value |
| O14520 | Aquaporin 7 (Aquaporin-7 like) | 1 . . . 272 | 254/272 (93%) | e−143 |
| | (Aquaporin adipose) (AQPap) - | 1 . . . 268 | 257/272 (94%) | |
| | Homo sapiens (Human), 342 aa. | | | |
| BAC05693 | Aquaporin adipose - Homo | 1 . . . 272 | 253/272 (93%) | e−142 |
| | sapiens (Human), 342 aa. | 1 . . . 268 | 256/272 (94%) | |
| Q8WX69 | BA251O17.3 (similar to aquaporin | 1 . . . 272 | 237/276 (85%) | e−130 |
| | 7) - Homo sapiens (Human), 346 | 1 . . . 272 | 243/276 (87%) | |
| | aa. | | | |
| O54794 | Aquaporin 7 - Mus musculus | 16 . . . 272 | 193/257 (75%) | e−108 |
| | (Mouse), 303 aa. | 1 . . . 253 | 218/257 (84%) | |
| AAM81581 | Aquaporin 7 variant - Rattus | 20 . . . 272 | 184/253 (72%) | e−106 |
| | norvegicus (Rat), 269 aa. | 4 . . . 252 | 216/253 (84%) | |

PFam analysis predicts that the NOV48a protein contains the domains shown in the Table 48E.

TABLE 48E
Domain Analysis of NOV48a

| | | Identities/ | |
|---|---|---|---|
| | | Similarities | |
| | NOV48a | for the | Expect |
| Pfam Domain | Match Region | Matched Region | Value |
| MIP | 27 . . . 251 | 71/247 (29%) | 1.5e−56 |
| | | 168/247 (68%) | |

### Example 49

The NOV49 clone was analyzed, and the nucleotide and encoded polypeptide sequences are shown in Table 49A.

TABLE 49A
NOV49 Sequence Analysis

| | SEQ ID NO:375 | 1461 bp | |
|---|---|---|---|
| NOV49a, | GAATTGAAGTGAATGGAACAGAAGCCAAGCAAGGTGGAGTGTGGGTCAGACCCAGAGG | | |
| CG173085-01 | | | |
| DNA Sequence | AGAACAGTGCCAGGTCACCAGATGGAAACCGAAAAAGAAAGAACGGCCAATGTTCCCT | | |
| | GAAAACCAGCATGTCAGGGTATATCCCTAGTTACCTGGACAAAGACGAGCAGTGTGTC | | |
| | GTGTGTGGGGACAAGGCAACTGGTTATCACTACCGCTGTATCACTTGTGAGGGCTGCA | | |
| | AGGGCTTCTTTCGCCGCACAATCCAGAAGAACCTCCATCCCACCTATTCCTGCAAATA | | |
| | TGACAGCTGCTGTGTCATTGACAAGATCACCCGCAATCAGTGCCAGCTGTGCCGCTTC | | |
| | AAGAAGTGCATCGCCGTGGGCATGGCCATGGACTTGGTTCTAGATGACTCGAAGCGGG | | |
| | TGGCCAAGCGTAAGCTGATTGAGCAGAACCGGGAGCGGCGGCGGAAGGAGGAGATGAT | | |
| | CCGATCACTGCAGCAGCGACCAGAGCCCACTCCTGAAGAGTGGGATCTGATCCACATT | | |
| | GCCACAGAGGCCCATCGCAGCACCAATGCCCAGGGCAGCCATTGGAAACAGAGGCGGA | | |
| | AATTCCTGCCCGATGACATTGGCCAGTCACCCATTGTCTCCATGCCGGACGGAGACAA | | |
| | GGTGGACCTGGAAGCCTTCAGCGAGTTTACCAAGATCATCACCCCGGCCATCACCCGT | | |
| | GTGGTGGACTTTGCCAAAAAACTGCCCATGTTCTCCGAGCTGCCTTGCGAAGACCAGA | | |
| | TCATCCTCCTGAAGGGGTGCTGCATGGAGATCATGTCCCTGCGGGCGGCTGTCCGCTA | | |
| | CGACCCTGAGAGCGACACCCTGACGCTGAGTGGGGAGATGGCTGTCAAGCGGGAGCAG | | |
| | CTCAAGAATGGCGGCCTGGGCGTAGTCTCCGACGCCATCTTTGAACTGGGCAAGTCAC | | |
| | TCTCTGCCTTTAACCTGGATGACACGGAAGTGGCTCTGCTGCAGGCTGTGCTGCTAAT | | |
| | GTCAACAGACCGCTCGGGCCTGCTGTGTGTGGACAAGATCGAGAAGAGTCAGGAGGCG | | |
| | TACCTGCTGGCGTTCGAGCACGACGTCAACCACCGCAAACACAACATTCCGCACTTCT | | |
| | GGCCCAAGCTGCTGATGAAGGGTCCGCAGGTCCGGCAGCTTGAGCAGCAGCTTGGTGA | | |
| | AGCGGGAAGTCTCCAAGGGCCGGTTCTTCAGCACCAGAGCCCGAAGAGCCCGCAGCAG | | |
| | CGTCTCCTGGAGCTGCTCCACCGAAGCGGAATTCTCCATGCCCGAGCGGTCTGTGGGG | | |
| | AAGACGACAGCAGTGAGGCGGACTCCCCGAGCTCCTCTGAGGAGGAACCGGAGGTCTG | | |
| | CGGGGACCTCGCAGGCAATGCAGCCTCTCCCTGAACCCCCCCAGAAGGCCGATGGGGA | | |
| | AGGAGAAGGAGTGCCATACCTTCTCCCAGGCCTCTGCCCCAAGAGCAGGAGGTGCCTG | | |
| | AAAGCTGGGAG | | |
| | ORF Start: ATG at 13 | | ORF Stop: TGA at 1366 |
| | SEQ ID NO:376 | 451 aa | MW at 506l2.1 kD |
| NOV49a, | MEQKPSKVECGSDPEENSARSPDGNRKRKNGQCSLKTSMSGYIPSYLDKDEQCVVCGD | | |
| CG173085-01 | | | |
| Protein Sequence | KATGYHYRCITCEGCKGFFRRTIQKNLHPTYSCKYDSCCVIDKITRNQCQLCRFKKCI | | |
| | AVGMAMDLVLDDSKRVAKRKLIEQNRERRRKEEMIRSLQQRPEPTPEEWDLIHIATEA | | |
| | HRSTNAQGSHWKQRRKFLPDDIGQSPIVSMPDGDKVDLEAFSEFTKIITPAITRVVDF | | |
| | AKKLPMFSELPCEDQIILLKGCCMEIMSLRAAVRYDPESDTLTLSGEMAVKREQLKNG | | |
| | GLGVVSDAIFELGKSLSAFNLDDTEVALLQAVLLMSTDRSGLLCVDKIEKSQEAYLLA | | |
| | FEHDVNHRKHNIPHFWPKLLMKGPQVRQLEQQLGEAGSLQGPVLQHQSPKSPQQRLLE | | |
| | LLHRSGILHARAVCGEDDSSEADSPSSSEEEPEVCGDLAGNAASP | | |
| | SEQ ID NO:377 | 1375 bp | |
| NOV49b, | CACCGGATCCACCATGGAACAGAAGCCAAGCAAGGTGGAGTGTGGGTCAGACCCAGAG | | |
| 311531811 DNA | | | |
| Sequence | GAGAACACTGCCAGGTCACCAGATGGAAAGCGAAAAAGAAAGAACGGCCAATGTTCCC | | |
| | TGAAAACCAGCATGTCAGGGTATATCCCTAGTTACCTGGACAAAGACGAGCAGTGTGT | | |
| | CGTGTGTGGGGACAAGGCAACTGGTTATCACTACCGCTGTATCACTTGTGAGGGCTGC | | |
| | AAGGGCTTCTTTCGCCGCACAATCCAGAAGAACCTCCATCCCACCTATTCCTGCAAAT | | |
| | ATGACAGCTGCTGTGTCATTGACAAGATCACCCGCAATCAGTGCCAGCTGTGCCGCTT | | |
| | CAAGAAGTGCATCGCCGTGGGCATGGCCATGGACTTGGTTCTAGATGACTCGAAGCGG | | |
| | GTGGCCAAGCGTAAGCTGATTGAGCAGAACCGGGAGCGGCGGCGGAAGGAGGAGATGA | | |
| | TCCGATCACTCCAGCAGCGACCAGAGCCCACTCCTGAAGAGTGGGATCTGATCCACAT | | |
| | TGCCACAGAGGCCCATCGCAGCACCAATGCCCAGGGCAGCCATTGGAAACAGAGGCGG | | |
| | AAATTCCTGCCCGATGACATTGGCCAGTCACCCATTGTCTCCATGCCGGACGGAGACA | | |
| | AGGTGGACCTGGAAGCCTTCAGCGAGTTTACCAAGATCATCACCCCGGCCATCACCCG | | |
| | TGTGGTGGACTTTGCCAAAAAACTGCCCATGTTCTCCGAGCTGCCTTGCGAAGACCAG | | |
| | ATCATCCTCCTGAAGGGGTGCTGCATGGAGATCATGTCCCTGCGGGCGGCTGTCCGCT | | |
| | ACGACCCTGAGAGCGACACCCTGACGCTGAGTGGGGAGATGGCTGTCAAGCGGGAGCA | | |
| | GCTCAAGAATGGCGGCCTGGGCGTAGTCTCCGACCCCATCTTTGAACTGGGCAAGTCA | | |
| | CTCTCTGCCTTTAACCTGGATGACACGGAAGTGGCTCTGCTGCAGGCTGTGCTGCTAA | | |
| | TGTCAACAGACCGCTCGGGCCTGCTGTGTGTGGACAAGATCGAGAAGAGTCAGGAGGC | | |
| | GTACCTGCTGGCGTTCGAGCACTACGTCAACCACCGCAAACACAACATTCCGCACTTC | | |
| | TGGCCCAAGCTGCTGATGAAGGGTCCGCAGGTCCGGCAGCTTGAGCAGCAGCTTGGTG | | |
| | AAGCGGGAAGTCTCCAAGGGCCGGTTCTTCAGCACCAGAGCCCGAAGAGCCCGCAGCA | | |
| | GCGTCTCCTGGAGCTGCTCCACCGAGCGGAATTCTCCATGCCCGAGCGGTCTTTGGTG | | |
| | GAAGACGACAGCAGTGAGGCGGACTCCCCGAGCTCCTCTGAGGAGGAACCGGAGGTCT | | |
| | GCGAGGACCTGGCAGGCAATGCAGCCTCTCCCGTCGACGGC | | |
| | ORF Start: at 2 | | ORF Stop: end of |
| | | | sequence |
| | SEQ ID NO:378 | 458 aa | MW at 51408.0 kD |

| | |
|---|---|
| NOV49b, | TGSTMEQKPSKVECGSDPEENSARSPDGKRKRKNGQCSLKTSMSGYIPSYLDKDEQCV |
| 311531811 | |
| Protein Sequence | VCGDKATGYHYRCITCEGCKGFFRRTIQKNLHPTYSCKYDSCCVIDKITRNQCQLCRF |
| | KKCIAVGMAMDLVLDDSKRVAKRKLIEQNRERRRKEEMIRSLQQRPEPTPEEWDLIHI |
| | ATEAHRSTNAQGSHWKQRRKFLPDDIGQSPIVSMPDGDKVDLEAFSEFTKIITPAITR |
| | VVDFAKKLPMFSELPCEDQIILLKGCCMEIMSLRAAVRYDPESDTLTLSGEMAVKREQ |
| | LKNGGLGVVSDAIFELGKSLSAFNLDDTEVALLQAVLLMSTDRSGLLCVDKIEKSQEA |
| | YLLAFEHYVNHRKHNIPHFWPKLLMKGPQVRQLEQQLGEAGSLQGPVLQHQSPKSPQQ |
| | RLLELLHRSGILHARAVFGEDDSSEADSPSSSEEEPEVCEDLAGNAASPVDG |

Sequence comparison of the above protein sequences yields the following sequence relationships shown in Table 49B.

TABLE 49B
Comparison of NOV49a against NOV49b.

| | | Identities/ |
|---|---|---|
| | | Similarities |
| | NOV49a Residues/ | for the |
| Protein Sequence | Match Residues | Matched Region |
| NOV49b | 1 . . . 451 | 447/451 (99%) |
| | 5 . . . 455 | 447/451 (99%) |

Further analysis of the NOV49a protein yielded the following properties shown in Table 49C.

TABLE 49C
Protein Sequence Properties NOV49a

| | |
|---|---|
| PSort | 0.9700 probability located in nucleus; 0.1000 |
| analysis: | probability located in mitochondrial matrix space; |
| | 0.1000 probability located in lysosome (lumen); |
| | 0.0000 probability located in endoplasmic reticulum |
| | (membrane) |
| SignalP | No Known Signal Sequence Predicted |
| analysis: | |

A search of the NOV49a protein against the Geneseq database, a proprietary database that contains sequences published in patents and patent publication, yielded several homologous proteins shown in Table 49D.

TABLE 49D
Geneseq Results for NOV49a

| | | NOV49a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAP80926 | Sequence of the human thyroid | 1 . . . 451 | 448/490 (91%) | 0.0 |
| | receptor hERBA 8.7 - Homo | 1 . . . 490 | 448/490 (91%) | |
| | sapiens, 490 aa. [WO8803168-A, | | | |
| | 05 MAY 1988] | | | |
| AAR26899 | HerbA-T sequence - Homo sapiens, | 1 . . . 451 | 446/490 (91%) | 0.0 |
| | 490 aa. [US5144007-A, 01 SEP. | 1 . . . 490 | 447/490 (91%) | |
| | 1992] | | | |
| AAY21630 | Ligand binding domain of nuclear | 1 . . . 377 | 369/377 (97%) | 0.0 |
| | receptor hTRalpha - Homo sapiens, | 1 . . . 377 | 371/377 (97%) | |
| | 410 aa. [WO9926966-A2, 03 JUN. | | | |
| | 1999] | | | |
| AAR78318 | Human thyroid hormone receptor | 1 . . . 377 | 369/377 (97%) | 0.0 |
| | alpha-1 - Homo sapiens, 410 aa. | 1 . . . 377 | 371/377 (97%) | |
| | [US5438126-A, 01 AUG. 1995] | | | |
| AAY21629 | Ligand binding domain of nuclear | 1 . . . 377 | 364/377 (96%) | 0.0 |
| | receptor rTRalpha - Rattus sp, 410 | 1 . . . 377 | 369/377 (97%) | |
| | aa. [WO9926966-A2, 03 JUN. | | | |
| | 1999] | | | |

In a BLAST search of public sequence datbases, the NOV49a protein was found to have homology to the proteins shown in the BLASTP data in Table 49E.

TABLE 49E
Public BLASTP Results for NOV49a

| | | NOV49a | Identities/ | |
|---|---|---|---|---|
| Protein | | Residues/ | Similarities | |
| Accession | | Match | for the | Expect |
| Number | Protein/Organism/Length | Residues | Matched Portion | Value |
| AAH35137 | Similar to thyroid hormone | 1 . . . 451 | 448/451 (99%) | 0.0 |
| | receptor - Homo sapiens (Human), | 1 . . . 451 | 448/451 (99%) | |
| | 451 aa. | | | |
| P10827 | Thyroid hormone receptor alpha | 1 . . . 451 | 448/490 (91%) | 0.0 |
| | (C-erbA-alpha) (c-erbA-1) (EAR- | 1 . . . 490 | 448/490 (91%) | |
| | 7) (EAR7) - Homo sapiens | | | |
| | (Human), 490 aa. | | | |
| O97716 | Thyroid hormone receptor alpha | 1 . . . 445 | 434/484 (89%) | 0.0 |
| | (C-erbA-alpha) (c-erbA-1) - Sus | 1 . . . 484 | 439/484 (90%) | |
| | scrofa (Pig), 506 aa. | | | |
| I57696 | c-erbA-alpha-2-related protein - | 1 . . . 451 | 435/492 (88%) | 0.0 |
| | rat, 492 aa. | 1 . . . 492 | 441/492 (89%) | |
| S14418 | thyroid hormone receptor alpha-3 - | 1 . . . 413 | 407/413 (98%) | 0.0 |
| | mouse, 413 aa (fragment). | 1 . . . 413 | 410/413 (98%) | |

PFam analysis predicts that the NOV49a protein contains the domains shown in the Table 49F.

TABLE 49F
Domain Analysis of NOV49a

| | | Identities/ | |
|---|---|---|---|
| | | Similarities | |
| | NOV49a | for the | |
| Pfam Domain | Match Region | Matched Region | Expect Value |
| zf-C4 | 51 . . . 128 | 50/78 (64%) | 2e−52 |
| | | 71/78 (91%) | |
| hormone_rec | 223 . . . 408 | 58/212 (27%) | 7.2e−34 |
| | | 136/212 (64%) | |

### Example 50

The NOV50 clone was analyzed, and the nucleotide and encoded polypeptide sequences are shown in Table 50A.

TABLE 50A
NOV50 Sequence Analysis

| | SEQ ID NO:379 | 2174 bp | |
|---|---|---|---|
| NOV50a, | GCCCTTTATCGCCGGAATTCATGTGCAATACCAACATGTCTGTACCTACTGATGGTGC | | |
| CG173095-01 | | | |
| DNA Sequence | TGTAACCACCTCACAGATTCCAGCTTCGGAACAAGAGACCCTGGTTAGACCAAAGCCA | | |
| | TTGCTTTTGAAGTTATTAAAGTCTGTTGGTGCACAAAAAGACACTTATACTATGAAAG | | |
| | AGAGATGGAGTTTCACTATGTTGCCCAGGCTGGTCTGGAACTCCTGGGCTCAAGGGAT | | |
| | CTGCTTACCTCGGCCTCCTAAAGTGCTAGATTTACAGGTTCTTTTTTATCTTGGCCAG | | |
| | TATATTATGACTAAACGATTATATGATGAGAAGCAACAACATATTGTATATTGTTCAA | | |
| | ATGATCTTCTAGGAGATTTGTTTGGCGTGCCAAGCTTCTCTGTGAAAGAGCACAGGAA | | |
| | AATATATACCATGATCTACAGGAACTTGGTAGTAGTCAATCAGCAGGAATCATCGGAC | | |
| | TCAGGTACATCTGTGAGTGAGAACAGGTGTCACCTTGAAGGTGGGAGTGATCAAAAGG | | |
| | ACCTTGTACAAGAGCTTCAGGAAGAGAAACCTTCATCTTCACATTTGGTTTCTAGACC | | |
| | ATCTACCTCATCTAGAAAGAGAGCAATTAGTGAGACAGAAGAAAATTCAGATGAATTA | | |
| | TCTGGTGAACGACAAAGAAAACGCCACAAATCTGATAGTATTTCCCTTTCCTTTGATG | | |
| | AAAGCCTGGCTCTGTGTGTAATAAGGGAGATATGTTGTGAAAGAAGCAGTAGCAGTGA | | |
| | ATCTACAGGGACGCCATCGAATCCGGATCTTGATGCTGGTGTAAGTGAACATTCAGGT | | |
| | GATTGGTTGGATCAGGATTCAGTTTCAGATCAGTTTAGTGTAGAATTTGAAGTTGAAT | | |
| | CTCTCGACTCAGAAGATTATAGCCTTAGTGAAGAAGGACAAGAACTCTCAGATGAAGA | | |
| | TGATGAGGTATATCAAGTTACTGTGTATCAGGCAGGGGAGAGTGATACAGATTCATTT | | |
| | GAAGAAGATCCTGAAATTTCCTTAGCTGACTATTGGAAATGCACTTCATGCAATGAAA | | |
| | TGAATCCCCCCCTTCCATCACATTGCAACAGATGTTGGGCCCTTCGTGAGAATTGGCT | | |
| | TCCTGAAGATAAAGGGAAAGATAAAGGGGAAATCTCTGAGAAAGCCAAACTGGAAAAC | | |
| | TCAACACAAGCTGAAGACGGCTTTGATGTTCCTGATTGTAAAAAAACTATAGTGAATG | | |
| | ATTCCAGAGAGTCATGTGTTGAGGAAAATGATGATAAAATTACACAAGCTTCACAATC | | |
| | ACAAGAAAGTGAAGACTATTCTCAGCCATCAACTTCTACTAGCATTATTTATAGCAGC | | |
| | CAAGAAGATGTGAAAGAGTTTGAAAGGGAAGAAACCCAAGACAAAGAAGAGAGTGTGG | | |
| | AATCTAGTTTGCCCCTTAATGCCATTGAACCTTGTGTGATTTGTCAAGGTCGACCTAA | | |
| | AAATGGTTGCATTGTCCATGGCAAAACAGGACATCTTATGGCCTGCTTTACATGTGCA | | |
| | AAGAAGCTAAAGAAAAGGAATAAGCCCTGCCCAGTATGTAGACAACCAATTCAAATGA | | |
| | TTGTGCTAACTTATTTCCCCTAGTTGACCTGTCTATAAGAGAATTATATATTTCTAAC | | |
| | TATATAACCCTAGGAATTTAGACAACCTGAAATTTATTCACATATATCAAAGTGAGAA | | |
| | AATGCCTCAATTCACATAGATTTCTTCTCTTTAGTATAATTGACCTACTTTGGTAGTG | | |
| | GAATAGTGAATACTTACTATAATTTGACTTGAATATGTAGCTCATCCTTTACACCAAC | | |
| | TCCTAATTTTAAATAATTTCTACTCTGTCTTAAATGAGAAGTACTTGGTTTTTTTTTT | | |
| | CTTAAATATGTATATGACATTTAAATGTAACTTATTATTTTTTTTCAGACCGAGTCTT | | |
| | GCTCTGTTACCCAGGCTGGAGTGCAGTGGGTGATCTTGGCTCACTGCAAGCTCTGCCC | | |
| | TCCCCGGGTTCGCACCATTCTCCTGCCTCAGCCTCCCAATTAGCTTGGCCTACAGTCA | | |
| | TCTGCCACCACACCTGGCTAATTTTTTGTACTTTTAGTAGAGACAGGGTTTCACCGTG | | |
| | TTAGCCAGGATGGTCTCGATCTCCTGACCTCGTGATCCGCCCACCTCGGCCTCCCAAA | | |
| | GTGCTGGGATTACAGGCATGAGCCACCG | | |
| | ORF Start: ATG at 21 | | ORF Stop: TAG at 1587 |
| | SEQ ID NO:380 | 522 aa | MW at 58895.6 kD |
| NOV50a, | MCNTNMSVPTDGAVTTSQIPASEQETLVRPKPLLLKLLKSVGAQKDTYTMKERWSFTM | | |
| CG173095-01 | | | |
| Protein Sequence | LPRLVNNSWAQGICLPRPPKVLDLQVLFYLGQYIMTKRLYDEKQQHIVYCSNDLLGDL | | |
| | FGVPSFSVKEHRKIYTMIYRNLVVVNQQESSDSGTSVSENRCHLEGGSDQKDLVQELQ | | |
| | EEKPSSSHLVSRPSTSSRKRAISETEENSDELSGERQRKRHKSDSISLSFDESLALCV | | |
| | IREICCERSSSSESTGTPSNPDLDAGVSEHSGDWLDQDSVSDQFSVEFEVESLDSEDY | | |
| | SLSEEGQELSDEDDEVYQVTVYQAGESDTDSFEEDPEISLADYWKCTSCNEMNPPLPS | | |
| | HCNRCWQLRENWLPEDKGKDKGEISEKAKLENSTQAEEGFDVPDCKKTIVNDSRESCV | | |
| | EENDDKITQASQSQESEDYSQPSTSSSIIYSSQEDVKEFEREETQDKEESVESSLPLN | | |
| | AIEPCVICQGRPKNGCIVHGKTGHLMACFTCAKKLKKLKKPCPVCRQPIQMIVLTYFP | | |
| | SEQ ID NO:381 | 1607 bp | |
| NOV50b, | GCCCTTTATCGCCGGAATTCATGTGCAATACCAACATGTCTGTACCTACTGATGGTGC | | |
| CG173095-02 | | | |
| DNA Sequence | TGTAACCACCTCACAGATTCCAGCTTCGGAACAAGAGACCCTGGTTAGACCAAAGCCA | | |
| | TTGCTTTTGAAGTTATTAAAGTCTGTTGGTGCACAAAAAGACACTTATACTATGAAAG | | |
| | AGAGATGGAGTTTCACTATGTTGCCCAGGCTGGTCTGGAACTCCTGGGCTCAAGGGAT | | |
| | CTGCTTACCTCGGCCTCCTAAAGTGCTAGATTTACAGGTTCTTTTTTATCTTGGCCAG | | |
| | TATATTATGACTAAACGATTATATGATGAGAAGCAACAACATATTGTATATTGTTCAA | | |
| | ATGATCTTCTAGGAGATTTGTTTGGCGTGCCAAGCTTCTCTGTGAAAGAGCACAGGAA | | |
| | AATATATACCATGATCTACAGGAACTTGGTAGTAGTCAATCAGCAGGAATCATCGGAC | | |
| | TCAGGTACATCTGTGAGTGAGAACAGGTGTCACCTTGAAGGTGGGAGTGATCAAAAGG | | |
| | ACCTTGTACAAGAGCTTCAGGAAGAGAAACCTTCATCTTCACATTTGGTTTCTAGACC | | |
| | ATCTACCTCATCTAGAAGGAGAGCAATTAGTGAGACAGAAGAAAATTCAGATGAATTA | | |
| | TCTGGTGAACGACAAAGAAAACGCCACAAATCTGATAGTATTTCCCTTTCCTTTGATG | | |
| | AAAGCTTGGCTCTGTGTGTAATAAGGGAGATATGTTGTGAAAGAAGCGGTAGCAGTGA | | |
| | ATCTACAGGGACGCCATCGAATCCGGATCTTGATGCTGGTGTAAGTGAACATTCAGGT | | |
| | GATTGGTTGGATCAGGATTCAGTTTCAGATCAGTTTAGTGTAGAATTTGAAGTTGAAT | | |
| | CTCTCGACTCAGAAGATTATAGCCTTAGTGAAGAAGGACAAGAACTCTCAGATGAAGA | | |
| | TGATGAGGTATATCAAGTTACTGTGTATCAGGCAGGGGAGAGTGATACAGATTCATTT | | |
| | GAAGAAGATCCTGAAATTTCCTCAGCTGACTATTGGAAATGCACTTCATGCAATGAAA | | |
| | TGAATCCCCCCCTTCCATCACATTGCAACAGATGTTGGGCCCTTCGTGAGAATTGGCT | | |
| | TCCTGAAGATAAAGGGAAAGATAAAGGGGAAATCTCTGAGAAAGCCAAACTGGAAAAC | | |
| | TCAACACAAGCTGAAGAGGGCTTTGATGTTCCTGATTGTAAAAAAACTATAGTGAATG | | |
| | ATTCCAGAGAGTCATGTGTTGAGGAAAATGATGATAAAATTACACAAGCTTCACAATC | | |
| | ACAAGAAAGTGAAGACTATTCTCAGCCATCAACTTCTAGTAGCATTATTTATAGCAGC | | |
| | CAAGAAGATGTGAAAGAGTTTGAAAGGGAAGAAACCCAAGACAAAGAAGAGAGTGTGG | | |
| | AATCTAGTTTGCCCCTTAATGCCATTGAACCTTGTGTGATTTGCCAAGGTCGACCTAA | | |
| | AAATGGTTGCATTGTCCATGGCAAAACAGGACATCTTATGGCCTGCTTTACATGTGCA | | |
| | AAGAAGCTAAAGAAAAGGAATAAGCCCTGCCCTGTATGTAGACAACCAATTCAAATGA | | |
| | TTGTGCTAACTTATTTCTCCTGACTGCAGCCAAGCTAATTC | | |
| | ORF Start: ATG at 21 | | ORF Stop: TGA at 1587 |
| | SEQ ID NO:382 | 522 aa | MW at 58857.5 kD |

| | |
|---|---|
| NOV50b, | MCNTNMSVPTDGAVTTSQIPASEQETLVRPKPLLLKLLKSVGAQKDTYTMKERWSFTM |
| CG173095-02 | |
| Protein Sequence | LPRLVWNSWAQGICLPRPPKVLDLQVLFYLGQYIMTKRLYDEKQQHIVYCSNDLLGDL |
| | FGVPSFSVKEHRKIYTMIYRNLVVVNQQESSDSGTSVSENRCHLEGGSDQVQLVQELQ |
| | EEKPSSSHLVSRPSTSSRRRAISETEENSDELSGERQRKRHKSDSISLSFDESLALCV |
| | IREICCERSGSSESTGTPSNPDLDAGVSEHSGDWLDQDSVSDQFSVEFEVESLDSEDY |
| | SLSEEGQELSDEDDEVYQVTVYQAGESDTDSFEEDPEISSADYWKCTSCNEMNPPLPS |
| | HCNRCWALRENWLPEDKGKDKGEISEKAKLENSTQAEEGFDVPDCKKTIVNDSRESCV |
| | EENDDKITQASQSQESEDYSQPSTSSSIIYSSQEDVKEFEREETQDKEESVESSLPLN |
| | AIEPCVICQGRPKNGCIVHGKTGHLMACFTCAKKLKKRNKPCPVCRQPIQMIVLTYFS |

Sequence comparison of the above protein sequences yields the following sequence relationships shown in Table 50B.

TABLE 50B
Comparison of NOV50a against NOV50b.

| | | Identities/ |
|---|---|---|
| | | Similarities |
| | NOV50a Residues/ | for the |
| Protein Sequence | Match Residues | Matched Region |
| NOV50b | 1 . . . 521 | 518/521 (99%) |
| | 1 . . . 521 | 519/521 (99%) |

Further analysis of the NOV50a protein yielded the following properties shown in Table 50C.

TABLE 50C
Protein Sequence Properties NOV50a

| | |
|---|---|
| PSort | 0.6000 probability located in nucleus; 0.3000 |
| analysis: | probability located in microbody (peroxisome); |
| | 0.1000 probability located in mitochondrial matrix |
| | space; 0.1000 probability located in lysosome (lumen) |
| SignalP | No Known Signal Sequence Predicted |
| analysis: | |

A search of the NOV50a protein against the Geneseq database, a proprietary database that contains sequences published in patents and patent publication, yielded several homologous proteins shown in Table 50D.

TABLE 50D
Geneseq Results for NOV50a

| | | NOV50a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAO15376 | Human Dm2 (Hdm2) protein - | 1 . . . 522 | 490/522 (93%) | 0.0 |
| | Homo sapiens, 491 aa. | 1 . . . 491 | 491/522 (93%) | |
| | [US2002045192-A1, 18 APR. 2002] | | | |
| AAE22654 | Human Ring finger E3 ubiquitin | 1 . . . 522 | 490/522 (93%) | 0.0 |
| | ligase (Mdm2) protein - Homo | 1 . . . 491 | 491/522 (93%) | |
| | sapiens, 491 aa. [WO200197830- | | | |
| | A1, 27 DEC. 2001] | | | |
| AAB48284 | Human MDM2 protein - Homo | 1 . . . 522 | 490/522 (93%) | 0.0 |
| | sapiens, 491 aa. [WO200075184- | 1 . . . 491 | 491/522 (93%) | |
| | A1, 14 DEC. 2000] | | | |
| AAY96567 | MDM2 oncoprotein - Homo | 1 . . . 522 | 490/522 (93%) | 0.0 |
| | sapiens, 491 aa. [WO200031238- | 1 . . . 491 | 491/522 (93%) | |
| | A2, 02 JUN. 2000] | | | |
| AAW94304 | Human MDM2 - Homo sapiens, | 1 . . . 522 | 490/522 (93%) | 0.0 |
| | 491 aa. [US5858976-A, 12 JAN. | 1 . . . 491 | 491/522 (93%) | |
| | 1999] | | | |

In a BLAST search of public sequence datbases, the NOV50a protein was found to have homology to the proteins shown in the BLASTP data in Table 50E.

TABLE 50E
Public BLASTP Results for NOV50a

| | | NOV50a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q00987 | Ubiquitin-protein ligase E3 Mdm2 | 1 . . . 522 | 490/522 (93%) | 0.0 |
| | (EC 6.3.2.-) (p53-binding protein | 1 . . . 491 | 491/522 (93%) | |
| | Mdm2) (Oncoprotein Mdm2) | | | |
| | (Double minute 2 protein) (Hdm2) - | | | |
| | Homo sapiens (Human), 491 aa. | | | |
| P56951 | Ubiquitin-protein ligase E3 Mdm2 | 1 . . . 522 | 463/522 (88%) | 0.0 |
| | (EC 6.3.2.-) (p53-binding protein | 1 . . . 491 | 479/522 (91%) | |
| | Mdm2) (Oncoprotein Mdm2) | | | |
| | (Double minute 2 protean (Edm2) - | | | |
| | Equus caballus (Horse), 491 aa. | | | |
| Q9GMZ6 | MDM2 - Canis familiaris (Dog), 487 | 1 . . . 522 | 456/522 (87%) | 0.0 |
| | aa. | 1 . . . 487 | 466/522 (88%) | |
| P56950 | Ubiquitin-protein ligase E3 Mdm2 | 1 . . . 522 | 454/522 (86%) | 0.0 |
| | (EC 6.3.2.-) (p53-binding protein | 1 . . . 487 | 464/522 (87%) | |
| | Mdm2) (Oncoprotein Mdm2) | | | |
| | (Double minute 2 protein) (Cdm2) - | | | |
| | Canis familiaris (Dog), 487 aa. | | | |
| Q95KN5 | MDM2 - Canis familiaris (Dog), 487 | 1 . . . 522 | 453/522 (86%) | 0.0 |
| | aa. | 1 . . . 487 | 463/522 (87%) | |

PFam analysis predicts that the NOV50a protein contains the domains shown in the Table 50F.

TABLE 50F
Domain Analysis of NOV50a

| | | Identities/ | |
|---|---|---|---|
| | | Similarities | |
| | NOV50a | for the | |
| Pfam Domain | Match Region | Matched Region | Expect Value |
| MDM2 | 30 . . . 126 | 56/97 (58%) | 1e−39 |
| | | 82/97 (85%) | |
| zf-RanBP | 330 . . . 359 | 9/32 (28%) | 3.6e−08 |
| | | 26/32 (81%) | |
| zf-C3HC4 | 469 . . . 509 | 14/55 (25%) | 0.81 |
| | | 31/55 (56%) | |

### Example 51

The NOV51 clone was analyzed, and the nucleotide and encoded polypeptide sequences are shown in Table 51A.

TABLE 51A
NOV51 Sequence Analysis

| | SEQ ID NO:383 | 2066 bp | |
|---|---|---|---|
| NOV51a, | TATTTCAGAAAGCTTCAAGAACAAGCTGGAGAAGGGAAGAGTTATTCCTCCATATTCA | | |
| CG173173-01 | | | |
| DNA Sequence | CCTGCTTCAACTACTATTCTTATTGGGAATGGACAATGGAATGTTCTCTGGTTTTATC | | |
| | ATGATCAAAAACCTCCTTCTCTTTTGTATTTCCATGAACTTATCCAGTCACTTTGGCT | | |
| | TTTCACAGGTGCCAACCAGTTCAGTGAAAGATGAGACCAATGACAACATCACGATATT | | |
| | TACCAGGATCTTGGATGGGCTCTTGGATGGCTACGACAACAGACTTCGGCCCGGGCTG | | |
| | GGAGAGCGCATCACTCAGGTGAGGACCGACATCTACGTCACCAGCTTCGGCCCGGTGT | | |
| | CCGACACGGAAATGGAGTACACCATAGACGTGTTTTTCCGACAAGGCTGGAAAGATGA | | |
| | AAGGCTTCGGTTTAAGGGGCCCATGCAGCGCCTCCCTCTCAACACGTTCTTCCACAAC | | |
| | GGGAAGAAGTCCATCGCTCACAACATGACCACGCCCAACAAGCTGCTGCGGCTGGAGG | | |
| | ACCACGGCACCCTGCTCTACACCATGCGCTTGACCATCTCTGCAGAGTGCCCCATGCA | | |
| | GCTTGAGGACTTCCCGATGGATGCGCACGCTTGCCCTCTGAAATTTGGCAGCTATGCG | | |
| | TACCCTAATTCTGAAGTCGTTTACGTCTGGACCAACGGCTCCACCAAGTCGGTGGTGG | | |
| | TGGCGGAAGATGGCTCCAGACTGAACCAGTACCACCTGATGGGGCAGACGGTGGGCAC | | |
| | TGAGAACATCAGCACCAGCACAGGCGAATACACAATCATGACAGCTCACTTCCACCTG | | |
| | AAAAGGAAGATTGGCTACTTTGTCATCCAGACCTACCTTCCCTGCATAATGACCGTGA | | |
| | TCTTATCACAGGTGTCCTTTTGGCTGAACCGGGAATCAGTCCCAGCCAGGACAGTTTT | | |
| | TGGGGTCACCACGGTGCTGACCATGACGACCCTCAGCATCAGCGCCAGGAACTCTCTG | | |
| | CCCAAAGTGGCCTACGCCACCGCCATGGACTGGTTCATAGCTGTGTGCTATGCCTTCG | | |
| | TCTTCTCGGCGCTGATAGAGTTTGCCACGGTCAATTACTTTACCAAGAGAGGCTGGGC | | |
| | CTGGGATGGCAAAAAAGCCTTGGAAGCAGCCAAGATCAAGAAAAAGCGTGAAGTCATA | | |
| | CTAAATAAGTCAACAAACGCTTTTACAACTGGGAAGATGTCTCACCCCCCAAACATTC | | |
| | CGAAGGAACAGACCCCAGCAGGGACGTCGAATACAACCTCAGTCTCAGTAAAACCCTC | | |
| | TGAAGAGAAGACTTCTGAAAGCAAAAAGACTTACAACAGTATCAGCAAAATTGACAAA | | |
| | ATGTCCCGAATCGTATTCCCAGTCTTGTTCGGCACTTTCAACTTAGTTTACTGGGCAA | | |
| | CGTATTTGAATAGGGAGCCGGTGATAAAAGGAGCCGCCTCTCCAAAATAACCGGCCAC | | |
| | ACTCCCAAACTCCAAGACAGCCATACTTCCAGCGAAATGGTACCAAGGAGAGGTTTTG | | |
| | CTCACAGGGACTCTCCATATGTGAGCACTATCTTTCAGGAAATTTTTGCATGTTTAAT | | |
| | AATATGTACAAATAATATTGCCTTGATGTTTCTATATGTAACTTCAGATGTTTCCAAG | | |
| | ATGTCCCATTGATAATTCGAGCAAACAACTTTCTGGAAAAACAGGATACGATGACTGA | | |
| | CACTCAGATGCCCAGTATCATACGTTGATAGTTTACAAACAAGATACGTATATTTTTA | | |
| | ACTGCTTCAACTGTTACCTAACAATGTTTTTTATACTTCAAATGTCATTTCATACAAG | | |
| | TTTTCCCAGTGAATAAATATTTTAGGAAACTCTCCATGATTATTAGAAGACCAACTAT | | |
| | ATTGCGAGAAACAGAGATCATAAAGAGCACGTTTTCCATTATGAGGAAACTTGGACAT | | |
| | TTATGTACAAAATGAATTGCCTTTGATAATTCTTACTGTTCTGAAATTAGGAAAGTAC | | |
| | TTGCATGATCTTACACGAAGAAATAGAATAGGCAAACTTTTATGTAGGCAGATTAATA | | |
| | ACAGAAATACATCATATGTTAGATACACAAAATATT | | |
| | ORF Start: ATG at 87 | | ORF Stop: TAA at 1440 |
| | SEQ ID NO:384 | 451 aa | MW at 50844.0 kD |

| | |
|---|---|
| NOV51a, | MDNGMFSGFIMIKNLLLFCISMNLSSHFGFSQVPTSSVKDETNDNITIFTRILDGLLD |
| CG173173-01 | |
| Protein Sequence | GYDNRLRPGLGERITQVRTDIYVTSFGPVSDTEMEYTIDVFFRQGWKDERLRFKGPMQ |
| | RLPLNTFFHNCKKSIAHNMTTPNKLLRLEDDGTLLYTMRLTISAECPMQLEDFPMDAH |
| | ACPLKFGSYAYPNSEVVYVWTNGSTKSVVVAEDGSRLNQYHLMGQTVGTENISTSTGE |
| | YTIMTAMFHLKRKIGYFVIQTYLPCIMTVILSQVSFWLNRESVPARTVFGVTTVLTMT |
| | TLSISARNSLPKVAYATANDWFIAVCYAFVFSALIEFATVNYFTKRGWAWDGKKALEA |
| | AKIKKKREVILNKSTNAFTTGKMSHPPNIPKEQTPAGTSNTTSVSVKPSEEKTSESKK |
| | TYNSISKIDKMSRIVFPVLFGTFNLVYWATYLNREPVIKGAASPK |

Further analysis of the NOV51a protein yielded the following properties shown in Table 51B.

TABLE 51B
Protein Sequence Properties NOV51a

| | |
|---|---|
| PSort | 0.7073 probability located in microbody (peroxisome); |
| analysis: | 0.7000 probability located in plasma membrane; 0.4477 |
| | probability located in mitochondrial inner membrane; |
| | 0.2000 probability located in endoplasmic reticulum |
| | (membrane) |
| SignalP | Cleavage site between residues 32 and 33 |
| analysis: | |

A search of the NOV51a protein against the Geneseq database, a proprietary database that contains sequences published in patents and patent publication, yielded several homologous proteins shown in Table 51C.

TABLE 51C
Geneseq Results for NOV51a

| | | NOV51a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAR59864 | Human GABA receptor alpha5 | 1 . . . 451 | 449/462 (97%) | 0.0 |
| | subunit - Homo sapiens, 462 aa. | 1 . . . 462 | 450/462 (97%) | |
| | [WO9413799-A, 23 JUN. 1994] | | | |
| AAR31186 | GABA-A receptor alpha-5 subunit - | 1 . . . 451 | 449/462 (97%) | 0.0 |
| | Homo sapiens, 462 aa. | 1 . . . 462 | 450/462 (97%) | |
| | [WO9222652-A, 23 DEC. 1992] | | | |
| AAR59862 | Human GABA receptor alpha2 | 39 . . . 444 | 312/419 (74%) | 0.0 |
| | subunit - Homo sapiens, 451 aa. | 32 . . . 447 | 347/419 (82%) | |
| | [WO9413799-A, 23 JUN. 1994] | | | |
| AAR31184 | GABA-A receptor alpha-2 subunit - | 39 . . . 444 | 312/419 (74%) | 0.0 |
| | Homo sapiens, 451 aa. | 32 . . . 447 | 347/419 (82%) | |
| | [WO9222652-A, 23 DEC. 1992] | | | |
| ABG26224 | Novel human diagnostic protein | 29 . . . 446 | 310/441 (70%) | e−177 |
| | #26215 - Homo sapiens, 547 aa. | 102 . . . 542 | 345/441 (77%) | |
| | [WO200175067-A2, 11 OCT. | | | |
| | 2001] | | | |

In a BLAST search of public sequence datbases, the NOV51a protein was found to have homology to the proteins shown in the BLASTP data in Table 51D.

TABLE 51D
Public BLASTP Results for NOV51a

| | | | Identities/ | |
|---|---|---|---|---|
| Protein | | | Similarities for | |
| Accession | | NOV51a Residues/ | the Matched | Expect |
| Number | Protein/Organism/Length | Match Residues | Portion | Value |
| P31644 | Gamma-aminobutyric-acid receptor | 1 . . . 451 | 449/462 (97%) | 0.0 |
| | alpha-5 subunit precursor | 1 . . . 462 | 450/462 (97%) | |
| | (GABA(A) receptor) - Homo | | | |
| | sapiens (Human), 462 aa. | | | |
| B34130 | gamma-aminobutyric | 1 . . . 451 | 427/464 (92%) | 0.0 |
| | acid/benzodiazepine receptor alpha- | 1 . . . 464 | 437/464 (94%) | |
| | 5 chain precursor - rat, 464 aa. | | | |
| P19969 | Gamma-aminobutyric-acid receptor | 1 . . . 451 | 427/464 (92%) | 0.0 |
| | alpha-5 subunit precursor | 1 . . . 464 | 437/464 (94%) | |
| | (GABA(A) receptor) - Rattus | | | |
| | norvegicus (Rat), 464 aa. | | | |
| P26048 | Gamma-aminobutyric-acid receptor | 39 . . . 444 | 313/419 (74%) | 0.0 |
| | alpha-2 subunit precursor | 32 . . . 447 | 348/419 (82%) | |
| | (GABA(A) receptor) - Mus | | | |
| | musculus (Mouse), 451 aa. | | | |
| P23576 | Gamma-aminobutyric-acid receptor | 39 . . . 444 | 313/419 (74%) | e−180 |
| | alpha-2 subunit precursor | 32 . . . 447 | 347/419 (82%) | |
| | (GABA(A) receptor) - Rattus | | | |
| | norvegicus (Rat), 451 aa | | | |

PFam analysis predicts that the NOV51a protein contains the domains shown in the Table 51E.

TABLE 51E
Domain Analysis of NOV51a

| | | Identities/ | |
|---|---|---|---|
| | | Similarities for | |
| Pfam | NOV51a | the Matched | Expect |
| Domain | Match Region | Region | Value |
| Neur_chan_LBD | 49 . . . 246 | 68/267 (25%) | 9e−60 |
| | | 163/267 (61%) | |
| Neur_chan_memb | 253 . . . 434 | 39/291 (13%) | 1.6e−58 |
| | | 162/291 (56%) | |

### Example 52

The NOV52 clone was analyzed, and the nucleotide and encoded polypeptide sequences are shown in Table 52A.

TABLE 52A
NOV52 Sequence Analysis

| | SEQ ID NO:385 | 2266 bp | |
|---|---|---|---|
| NOV52a, | CTCGGGCCTGGGGCTCTGCCTGAACAACCGGCCCCCCAGACAGGACTTTGTGTACCCG | | |
| CG51213-01 | | | |
| DNA Sequence | ACAGTGGCACCGGGCCAAGCCTACGATGCAGATGAGCAATGCCGCTTTCAGCATGGAG | | |
| | TCAAATCGCGTCAGTTGGTGCTACAAACGGGTCTGTGTCCCCTTTGGGTCGCGCCCAG | | |
| | AGGGTGTGGACGGAGCCTGGGGGCCGTGGACTCCATGGGGCGACTGCAGCCGCACCTG | | |
| | TGGCGGCGGCGTGTCCTCTTCTAGCCGTCACTGCGACAGCCCCAGGCCAACCATCGGG | | |
| | GGCAAGTACTGTCTGGGTGAGAGAAGGCGGCACCGCTCCTGCAACACGGATGACTGTC | | |
| | CCCCTGGCTCCCAGGACTTCAGAGAAGTGCAGTGTTCTGAATTTGACAGCATCCCTTT | | |
| | CCGTGGGAAATTCTACAAGTGGAAAACGTACCGGGGAAGGTGAGTGTGGGACTCCAAA | | |
| | GGCTGTGGGGCCGTGAAGGCCAGCCGTGGGAGTGTCCAGCAGCAGGTGGATGAATGCA | | |
| | GCATCCCGGGGTCTGCCATGAGCCCTGTCCCCACCCGGGGAGACAGAGTACCTGGGAT | | |
| | ACGGTACCATGGGGGTTCAACGTGACGCTGGGAGCCCCCACTCCCTCTGCCCAAGCTG | | |
| | CCCTTCCTCTTGGGTCTGGGGTCTGTCCCTCTTGGCCTCACTCCCCCAGGGAGCAAGC | | |
| | AAAGAGTTCCGGGGTGGCCTGGCCCGTGGTGTGACGGGGCCGTGCCCCCCAGGGGGCG | | |
| | TGAAGGCCTGCTCGCTCACGTGCCTAGCGGAAGGCTTCAACTTCTACACGGAGAGGGC | | |
| | GGCAGCCGTGGTGGACGGGACACCCTGCCGTCCAGACACGGTGGACATTTGCGTCAGT | | |
| | GGCGAATGCAAGCACGTGGGCTGCGACCGAGTCCTGGGCTCCGACCTGCGGGAGGACA | | |
| | AGTGCCGAGTGTGTGGCGGTGACGGCAGTGCCTGCGAGACCATCGAGGGCGTCTTCAG | | |
| | CCCAGCCTCACCTGGGGCCGGGTACGAGGATGTCGTCTGGATTCCCAAAGGCTCCGTC | | |
| | CACATCTTCATCCAGGATCTGAACCTCTCTCTCAGTCACTTGGCCCTGAAGGGAGACC | | |
| | AGGAGTCCCTGCTGCTGGAGGGGCTGCCTGGGACCCCCCAGCCCCACCGTCTGCCTCT | | |
| | AGCTGGGACCACCTTTCAACTGCGACAGGGGCCAGACCAGGTCCAGAGCCTCGAAGCC | | |
| | CTGGGACCGATTAATGCATCTCTCATCGTCATGGTGCTGGCCCGGACCGAGCTGCCTG | | |
| | CCCTCCGCTACCGCTTCAATGCCCCCATCGCCCGTGACTCGCTGCCCCCCTACTCCTG | | |
| | GCACTATGCGCCCTGGACCAAGTGCTCGGCCCAGTGTGCAGGCGGTAGCCAGGTGCAG | | |
| | GCGGTGGAGTGCCGCAACCAGCTGGACAGCTCCGCGGTCGCCCCCCACTACTGCAGTG | | |
| | CCCACAGCAAGCTGCCCAAAAGGCAGCGCGCCTGCAACACGGAGCCTTGCCCTCCAGA | | |
| | CTGGGTTGTAGGGAACTGGTCGCTCTGCAGCCGCAGCTGCGATGCAGGCGTGCGCAGC | | |
| | CGCTCGGTCGTGTGCCAGCGCCGCGTCTCTGCCGCGGAGGAGAAGGCGCTGGACGACA | | |
| | GCGCATGCCCGCAGCCGCGCCCACCTGTACTGGAGGCCTGCCACGGCCCCACTTGCCC | | |
| | TCCGGAGTGGGCGGCCCTCGACTGGTCTGAGTGCACCCCCAGCTGCGGGCCGGGCCTC | | |
| | CGCCACCGCGTGGTCCTTTGCAAGAGCGCAGACCACCGCGCCACGCTGCCCCCGGCGC | | |
| | ACTGCTCACCCGCCGCCAAGCCACCGGCCACCATGCGCTGCAACTTGCGCCGCTGCCC | | |
| | CCCGGCCCGCTGGGTGGCTGGCGAGTGGGGTGAGTGCTCTGCACAGTGCGGCGTCGGG | | |
| | CAGCGGCAGCGCTCGGTGCGCTGCACCAGCCACACGGGCCAGGCGTCGCACGAGTGCA | | |
| | CGGAGGCCCTGCGGCCGCCCACCACGCAGCAGTGTGAGGCCAAGTGCGACAGCCCAAC | | |
| | CCCCGGGGACGGCCCTGAAGAGTGCAAGGATGTGAACAAGGTCGCCTACTGCCCCCTG | | |
| | GTGCTCAAATTTCAGTTCTGCAGCCGAGCCTACTTCCGCCAGATGTGCTGCAAAACCT | | |
| | GCCAGGGCCACTAGGGGGCGCGCGGCACCCGGAGCCACAGCTGGCGGGGTCTCCGCCG | | |
| | CCAGCCCTGCAGCTGGGCCGGCCAGAGGGGGCCCCGGGAAGGCGGGAACTGGGAGGGA | | |
| | AGGG | | |
| | ORF Start: ATG at 589 | | ORF Stop: TAG at 2158 |
| | SEQ ID NO:386 | 523 aa | MW at 56126.2 kD |
| NOV52a, | MGVQRDAGSPHSLCPSCPSSWVWGLSLLASLPQGASKEFRGGLARGVTGPCPPGGVKA | | |
| CG51213-01 | | | |
| Protein Sequence | CSLTCLAEGFNFYTERAAAVVDGTPCRPDTVDICVSGECKHVGCDRVLGSDLREDKCR | | |
| | VCGGDGSACETIEGVFSPASPGAGYEDVVWIPKGSVHIFIQDLNLSLSHLALKGDQES | | |
| | LLLEGLPGTPQPHRLPLAGTTFQLRQGPDQVQSLEALGPTNASLIVMVLARTELPALR | | |
| | YRFNAPIARDSLPPYSWHYAPWTKCSAQCAGGSQVQAVECRNQLDSSAVAPHYCSAHS | | |
| | KLPKRQRACNTEPCPPDWVVGNWSLCSRSCDAGVRSRSVVCQRRVSAAEEKALDDSAC | | |
| | PQPRPPVLEACHGPTCPPEWAALDWSECTPSCGPGLRHRVVLCKSADHRATLPPAHCS | | |
| | PAAKPPATMRCNLRRCPPARWVAGEWGECSAQCGVGQRQRSVRCTSHTGQASHECTEA | | |
| | LRPPTTQQCEAKCDSPTPGDGPEECKDVNKVAYCPLVLKFQFCSRAYFRQMCCKTCQG | | |
| | H | | |
| | SEQ ID NO:387 | | 1866 bp |
| NOV52b, | TCCATAAATGGACCTTATTGGGAGAGTATAAGTCACAGGCCATGCCCCGCAAGGGGAT | | |
| CG51213-07 | | | |
| DNA Sequence | GCACGAAGACCCACCGCGAGCCAGGAAGGGAGCACCGGGCTCTCTGCTCTGGGACCGG | | |
| | CAGTGAGCCGGACATCTGGGTCCTCCCAAGCCGGGCGGGCTGCCCCAGGGAGGAAGGG | | |
| | AGGGGGGCGAGCCTGAGCGGGCACCTCGGCCCGCAGGAGGTCTGCAGCGAGCTGTGGT | | |
| | GTCTGAGCAAGAGCAACCGGTGCATCACCAACAGCATCCCGGCCGCCGAGGGCACGCT | | |
| | GTGCCAGACGCACACCATCGACAAGGGGTGGTGCTACAAACGGGTCTGTGTCCCCTTT | | |
| | GGGTCGCGCCCAGAGGGTGTGGACGGAGCCTGGGGGCCGTGGACTCCATGGGGCGACT | | |
| | GCAGCCGGACCTGTGGCGGCGGCGTGTCCTCTTCTAGCCGTCACTGCGACAGCCCCAG | | |
| | GCCAACCATCGGGGGCAAGTACTGTCTGGGTGAGAGAAGGCGGCACCGCTCCTGCAAC | | |
| | ACGGATGACTGTCCCCCTGGCTCCCAGGACTTCAGAGAAGTGCAGTGTTCTGAATTTG | | |
| | ACAGCATCCCTTTCCGTGGGAAATTCTACAAGTGGAAAACGTACCGGGGAGGGGGCGT | | |
| | GAAGGCCTGCTCGCTCACGTGCCTAGCGGAAGGCTTCAACTTCTACACGGAGAGGGCG | | |
| | GCAGCCGTGGTGGACGGGACACCCTGCCGTCCAGACACGGTGGACATTTGCGTCAGTG | | |
| | GCGAATGCAAGCACGTGGGCTGCGACCGAGTCCTGGGCTCCGACCTGCGGGAGGACAA | | |
| | GTGCCGAGTGTGTGGCGGTGACGGCAGTGCCTGCGAGACCATCGAGGGCGTCTTCAGC | | |
| | CCAGCCTCACCTGGGGCCGGGTACGAGGATGTCGTCTGGATTCCCAAAGGCTCCGTCC | | |
| | ACATCTTCATCCAGGATCTGAACCTCTCTCTCAGTCACTTGGCCCTGAAGGGAGACCA | | |
| | GGAGTCCCTGCTGCTGGAGGGGCTGCCCGGGACCCCCCAGCCCCACCGTCTGCCTCTA | | |
| | GCTGGGACCACCTTTCAACTGCGACAGGGGCCAGACCAGGTCCAGAGCCTCGAAGCCC | | |
| | TGGGACCGATTAATGCATCTCTCATCGTCATGGTGCTGGCCCGGACCGAGCTGCCTGC | | |
| | CCTCCGCTACCGCTTCAATGCCCCCATCGCCCGTGACTCGCTGCCCCCCTACTCCTGG | | |
| | CACTATGCGCCCTGGACCAAGTGCTCGGCCCAGTGTGCAGGCGGTAGCCAGGTGCAGG | | |
| | CGGTGGAGTGCCGCAACCAGCTGGACAGCTCCGCGGTCGCCCCCCACTACTGCAGTGC | | |
| | CCACAGCAAGCTGCCCAAAAGGCAGCGCGCCTGCAACACGGAGCCTTGCCCTCCAGAC | | |
| | TGGGTTGTAGCGAACTGGTCGCTCTGCAGCCGCAGCTGCGATGCAGGCGTGCGCAGCC | | |
| | GCTCGGTCGTGTGCCAGCGCCGCGTCTCTGCCGCGGAGGAGAAGGCGCTGGACGACAG | | |
| | CGCATGCCCGCAGCCGCGCCCACCTGTACTGGAGGCCTGCCACGGCCCCACTTGCCCT | | |
| | CCGGAGTGGGCGGCCCTCGACTGGTCTGAGTGCACCCCCAGCTGCGGGCCGGGCCTCC | | |
| | GCCACCGCGTGGTCCTTTGCAAGAGCGCAGACCACCGCGCCACGCTGCCCCCGGCGCA | | |
| | CTGCTCACCCGCCGCCAAGCCACCGGCCACCATGCGCTGCAACTTGCGCCGCTGCCCC | | |
| | CCGGCCCGCTGGGTGGCTGGCGAGTGGGGTGAGTGCTCTGCACAGTGCGGCGTCGGGC | | |
| | AGCGGCAGCGCTCGGTGCGCTGCACCAGCCACACGGGCCAGGCGTCGCACGAGTGCAC | | |
| | GGAGGCCCTG | | |
| | ORF Start: at 1 | | ORF Stop: end of |
| | | | sequence |
| | SEQ ID NO:388 | 622 aa | MW at 67376.2 kD |
| NOV52b, | SINGAYWESISHRPCPARGCTKTHREPGREHRALCSGTGSEPDIWVLPSRAGCPREEG | | |
| CG51213-07 | | | |
| Protein Sequence | RGASLSGHLGPQEVCSELWCLSKSNRCITNSIPAAEGTLCQTHTIDKGWCYKRVCVPF | | |
| | GSRPEGVDGAWGPWTPWGDCSRTCGGGVSSSSRHCDSPRPTIGGKYCLGERRRHRSCN | | |
| | TDDCPPGSQDFREVQCSEFDSIPFRGKFYKWKTYRGGGVKACSLTCLAEGFNFYTERA | | |
| | AAVVDGTPCRPDTVDICVSGECKHVGCDRVLGSDLREDKCRVCGGDGSACETIEGVFS | | |
| | PASPGAGYEDVVWIPKGSVHIFIQDLNLSLSHLALKGDQESLLLEGLPGTPQPHRLPL | | |
| | AGTTFQLRQGPDQVQSLEALGPINASLIVMVLARTELPALRYRFNAPIARDSLPPYSW | | |
| | HYAPWTKCSAQCAGGSQVQAVECRNQLDSSAVAPHYCSAHSKLPKRQRACNTEPCPPD | | |
| | WVVGNWSLCSRSCDAGVRSRSVVCQRRVSAAEEKALDDSACPQPRPPVLEACHGPTCP | | |
| | PEWAALDWSECTPSCGPGLRHRVVLCKSADHRATLPPAHCSPAAKPPATMRCNLRRCP | | |
| | ARWVACEWGECSAQCGVGQRQRSVRCTSHTCQASHECTEAL | | |
| | SEQ ID NO:389 | 3199 bp | |
| NOV52c, | TAAAGGCTTCAGCCTGGTGCCTGGTCCAGAGATAGTGGTGGTCATTGTTACCCCATAA | | |
| CG51213-02 | | | |
| DNA Sequence | TGGCATTGGTGCAAGTCCTTTCTTATCTATCCTGTCACGTGCCTCATAGCCATTTATA | | |
| | TAGGCAAGACAGGCATTAGGCTGCCCATCTTGTAGATGAGTAAACTGAGGCCCAGAGA | | |
| | GGGGAAATATATTGCAAGTTGGTAGCAGAATTGAGGTCTCTGCACAACTCAAATATGC | | |
| | CACAGTGCCTCCTTGTGGAGAGGAGGACAAAAGCAGAGCTGAAATCATTATCTTGAAG | | |
| | AGGTGTCAGAAGTGGGATTGCGACAGGACTGATGTGATATTTTTAGATATGGCCAAGA | | |
| | GGACACAGTCTGAGTTTTTAGCTGAGAAATGTCCTCTATAAGGCAGAAGGCAGAGATT | | |
| | CTAGAGGACCTTTGAGGGAGAATGTATTTGAGAACAACTCTTCCAGCTTCTTACATAT | | |
| | GTACAGGTATCTCTCAGGGGCTGACCTAGGAAGGGTCCTTTCCTGTGGCCATTGATCG | | |
| | ATCCAGTCCCACATCTGGAAAGCTTACAAGAATTGGGTTCAAAGCGGGGATTACACTT | | |
| | GATAATTACAGAAGGACCACCTACTTCTTAGAGGAAAGACGCTCGGAGGTTGCTTAGG | | |
| | ATGTGGGCCAAGAGCGTCAGAGAGGACCACCTACTTTTTAGAGGAAAGACGCTGGGAG | | |
| | GTTGCTTAGGATGTGGGCCAAGAGGGTCAGAGATTTTGCTTCACCTGAACTCACTGGG | | |
| | GCTTCTCCAGGGATATTAACCTGGACTTTAAGAGTCACAGTGAGTCCCTGGGACTAGT | | |
| | TCAGCCCATCCAGGATTCAGACGGGAAGAAGGTGGGGCTGATTTTTCACCTGGAGAAA | | |
| | GAGAGGCATGTCCCACACAGACCTAACTCGGCATTGTCCCCTCCCAAACTCCCACCCC | | |
| | TCCACATAGCTTAAAAGTGTTGGGGGCTTCTCCAGTTTAGATGGGGGAACAAAGAGAA | | |
| | CCAACAGCTGGAAAAAACTAGAGATGAGGCCGTTGGCCTAGTCATCATCCAGGCCGAT | | |
| | TTCTCAGAACCACCACTTTCTCTTCGGCTACTTTGCCCATCCCATAAAAGAACCCCAA | | |
| | ATCCTTCCTGTTCATTCCTCACCAGTTCCCACGTTTCCTTCCAGAAACTCAGAAGGCA | | |
| | CCAGGAACTGAATTGCAAAGTTCGTTAGAGCACAGACTCTGAATTAAAGAGCTGGGTT | | |
| | AAACTCCAGGCTATTCCCTTAGTAGCTGTGTGACCTTACCTGTCTGAAGCTTGGTTTT | | |
| | CTCCCAGTAAGATGGGGTAGTACTGCCTAAAGAGGTATATGGCATGTATAAAGTGCTC | | |
| | CATAAATGGAGCTTATTGGGAGAGTATAAGTCACAGGCCATGCCCCGCAAGGGGATGC | | |
| | ACGAAGACCCACCGCGAGCCAGGAAGGGAGCACCGGGCTCTCTGCTCTGGGACCGGCA | | |
| | GTGAGCCGGACATCTGGGTCCTCCCAAGCCGGGCGGGCTGCCCCAGGGAGGAAGGGAG | | |
| | GGGGGCGAGCCTGAGCGGGCACCTCGGCCCGCAGGAGGTCTGCAGCGAGCTGTGGTGT | | |
| | CTGAGCAAGAGCAACCGGTGCATCACCAACAGCATCCCGGCCGCCGAGGGCACCCTGT | | |
| | GCCAGACGCACACCATCGACAAGGGGTGGTGCTACAAACGGGTCTGTGTCCCCTTTGG | | |
| | GTCGCGCCCAGAGGGTGTGGACGGAGCCTGGGGGCCGTGGACTCCATGGGGCGACTGC | | |
| | AGCCGGACCTGTGGCGGCGGCGTGTCCTCTTCTAGCCGTCACTGCGACAGCCCCAGGC | | |
| | CAACCATCGGGGGCAAGTACTGTCTGGGTGAGAGAAGGCGGCACCGCTCCTGCAACAC | | |
| | GGATGACTGTCCCCCTGGCTCCCAGGACTTCAGAGAAGTGCAGTGTTCTGAATTTGAC | | |
| | AGCATCCCTTTCCGTGGGAAATTCTACAAGTGGAAAACGTACCGGGGAGGGGGCGTGA | | |
| | AGGCCTGCTCGCTCACGTGCCTAGCGGAAGGCTTCAACTTCTACACGGAGAGGGCGGC | | |
| | AGCCGTGGTGGACGGGACACCCTGCCGTCCAGACACGGTCGACATTTGCGTCAGTGGC | | |
| | GAATGCAAGCACGTGGGCTGCGACCGAGTCCTGGGCTCCGACCTGCGGGAGGACAAGT | | |
| | GCCGAGTGTGTGGCGGTGACGGCAGTGCCTGCGAGACCATCGAGGGCGTCTTCAGCCC | | |
| | AGCCTCACCTGGGGCCGGGTACGAGGATGTCGTCTGGATTCCCAAAGGCTCCGTCCAC | | |
| | ATCTTCATCCAGGATCTCAACCTCTCTCTCAGTCACTTGGCCCTGAAGGGAGACCAGG | | |
| | AGTCCCTGCTGCTGGAGGGGCTGCCCGGGACCCCCCAGCCCCACCGTCTGCCTCTAGC | | |
| | TGGGACCACCTTTCAACTGCGACAGGGGCCAGACCAGGTCCAGAGCCTCGAAGCCCTG | | |
| | GGACCGATTAATGCATCTCTCATCGTCATGGTGCTGGCCCGGACCGAGCTGCCTGCCC | | |
| | TCCGCTACCGCTTCAATGCCCCCATCGCCCGTGACTCGCTGCCCCCCTACTCCTGGCA | | |
| | CTATGCGCCCTGGACCAAGTGCTCGGCCCAGTGTGCAGGCGGTAGCCAGGTGCAGGCG | | |
| | GTGGAGTGCCGCAACCAGCTGGACAGCTCCGCGGTCGCCCCCCACTACTGCAGTGCCC | | |
| | ACAGCAAGCTGCCCAAAAGGCAGCGCGCCTGCAACACGGAGCCTTGCCCTCCAGACTG | | |
| | GGTTGTAGGGAACTGGTCGCTCTGCAGCCGCAGCTGCGATGCAGGCGTGCGCAGCCGC | | |
| | TCGGTCGTGTGCCAGCGCCGCGTCTCTGCCGCGGAGGAGAAGGCGCTGGACCACAGCG | | |
| | CATGCCCGCAGCCGCGCCCACCTGTACTGGAGGCCTGCCACGGCCCCACTTGCCCTCC | | |
| | GGAGTGGGCGGCCCTCGACTGGTCTGAGTGCACCCCCAGCTGCGGGCCCCGCCTCCGC | | |
| | CACCGCGTGGTCCTTTGCAAGAGCGCAGACCACCGCGCCACGCTGCCCCCGGCGCACT | | |
| | GCTCACCCGCCGCCAAGCCACCGGCCACCATGCGCTGCAACTTGCGCCGCTGCCCCCC | | |
| | GGCCCGCTGGGTGGCTGGCGAGTGGGGTGAGTGCTCTGCACAGTGCGGCGTCGGGCAG | | |
| | CGGCAGCGCTCGGTGCGCTGCACCAGCCACACGGGCCAGGCGTCGCACGAGTGCACGG | | |
| | AGGCCCTGC | | |
| | ORF Start: at 1297 | | ORF Stop: at 3199 |
| | SEQ ID NO:390 | 634 aa | MW at 68853.1 kD |
| NOV52c, | YCLKRYMACIKCSINGAYWESISHRPCPARGCTKTHREPGREHRALCSGTGSEPDINV | | |
| CG51213-02 | | | |
| Protein Sequence | LPSRAGCPREEGRGASLSGHLGPQEVCSELWCLSKSNRCITNSIPAAEGTLCQTHTID | | |
| | KGWCYKRVCVPFGSRPEGVDGAWGPWTPWGDCSRTCGGGVSSSSRHCDSPRPTIGGKY | | |
| | CLGERRRHRSCNTDDCPPGSQDFREVQCSEFDSIPFRGKFYKWKTYRGGGVKACSLTC | | |
| | LAEGFNFYTERAAAVVDGTPCRPDTVDICVSGECKHVGCDRVLGSDLREDKCRVCGGD | | |
| | GSACETIEGVFSPASPGAGYEDVVWIPKGSVHIFIQDLNLSLSHLALKGDQESLLLEG | | |
| | LPGTPQPHRLPLAGTTFQLRQGPDQVQSLEALGPINASLIVMVLARTELPALRYRFNA | | |
| | PIARDSLPPYSWHYAPWTKCSAQCAGGSQVQAVECRNQLDSSAVAPHYCSAHSKLPKR | | |
| | QRACNTEPCPPDWVVGNWSLCSRSCDAGVRSRSVVCQRRVSAAEEKALDDSACPQPRP | | |
| | PVLEACHGPTCPPEWAALDWSECTPSCGPGLRHRVVLCKSADHRATLPPAHCSPAAKP | | |
| | PATMRCNLRRCPPARWVAGEWGECSAQCGVGQRQRSVRCTSHTGQASHECTEAL | | |
| | SEQ ID NO:391 | 3700 bp | |
| NOV52d, | CTGACATTCCACCCTTGACACCCCCCAACATCCTAACTTAGCTGGTAACTGCAGCACC | | |
| CG51213-03 | | | |
| DNA Sequence | CTCTAAGGAATTCCTAAAGAATTCTGAAGCTACTCCTCAACATCTGCTGTGACCCAGG | | |
| | TATCCTAACAATGATCATGGTGTCTGACATTTACTGAGCTCTCACTATGGGCTAAGCA | | |
| | TGTGCTGTGTGTCACCATCTAAACTCCTGACAATCCTGCTAGCCCCCACGTTACAGAG | | |
| | GAAGGGACTGAGCCATAGCATAGGGAGGATGACTTGTCCAAGGCCACAGTTTGAGACC | | |
| | ATGACAGAGCTGGGATTTAAATCCAGGTCTCTCATGACTCTCTAAATTTTACAAAGGG | | |
| | GCAGGGGAGGGGAGGAGCTGTCAAAATATCAAGCTTGGGCTGGCACTGGCTATATGTT | | |
| | GAATTGAGCCTTCCTTTTAGTTTTTGAAGGAACATCTTTCAGGCCATCTTGGCAAAGG | | |
| | GGGATTTATTTACTAAATGTGAACTGGTTAATATATGTAAAGGGTTCAGCCTGGTGCC | | |
| | TGGTCCAGAGATAGTGGTGGTCATTGTTACCCCATAATGGCATTGGTGCAAGTCCTTT | | |
| | CTTATCTATCCTGTCACGTGCCTCATAGCCATTTATATAGGCAAGACAGGCATTAGGC | | |
| | TGCCCATCTTGTAGATGAGTAAACTGAGGCCCAGAGAGGGGAAATATATTGCAAGTTG | | |
| | GTAGCAGAATTGAGGTCTCTGCACAACTCAAATATGCCACAGTGCCTCCTTGTGGAGA | | |
| | GGAGGACAAAAGCAGAGCTGAAATCATTATCTTGAAGAGGTGTCAGAAGTGGGATTGC | | |
| | GACAGGACTGATGTGATATTTTTAGATATGGCCAAGAGGACACAGTCTGAGTTTTTAG | | |
| | CTGAGAAATGTCCTCTATAAGGCAGAAGGCAGAGATTCTAGAGGACCTTTGAGGGAGA | | |
| | ATGTATTTGAGAACAACTCTTCCAGCTTCTTACATATGTACAGGTATCTCTCAGGGGC | | |
| | TGACCTAGGAAGGGTCCTTTCCTGTGGCCATTGATCGATCCAGTCCCACATCTGGAAA | | |
| | GCTTACAAGAATTGGGTTCAAAGCGGGGATTACACTTGATAATTACAGAAGGACCACC | | |
| | TACTTCTTAGAGGAAAGACGCTGGGAGGTTGCTTAGGATGTGGGCCAAGAGGGTCAGA | | |
| | GAGGACCACCTACTTTTTAGAGGAAAGACGCTGGGAGGTTGCTTAGGATGTGGGCCAA | | |
| | GAGGGTCAGAGATTTTGCTTCACCTGAACTCACTGGGGCTTCTCCAGGGATATTAACC | | |
| | TGGACTTTAAGAGTCAGAGTGAGTCCCTGGGACTAGTTCAGCCCATCCAGGATTCAGA | | |
| | CGGGAAGAAGGTGGGGCTGATTTTTCACCTGGAGAAAGAGAGGCATGTCCCACACAGA | | |
| | CCTAACTCGGCATTGTCCCCTCCCAAACTCCCACCCCTCCACATAGCTTAAAAGTGTT | | |
| | GGGCGCTTCTCCAGTTTAGATGGGGGAACAAAGAGAACCAACAGCTGGAAAAAACTAG | | |
| | AGATGAGGCCGTTGGCCTAGTCATCATCCAGGCCGATTTCTCAGAACCACCACTTTCT | | |
| | CTTCGGCTACTTTGCCCATCCCATAAAAGAACCCCAAATCCTTCCTGTTCATTCCTCA | | |
| | GCAGTTCCCACGTTTCCTTCCAGAAACTCAGAAGGCACCAGGAACTGAATTGCAAAGT | | |
| | TCGTTAGAGCACAGACTCTGAATTAAAGAGCTGGGTTAAACTCCAGGCTATTCCCTTA | | |
| | GTAGCTGTGTGACCTTACCTGTCTGAAGCTTGGTTTTCTCCCAGTAAGATGGGGTAGtT | | |
| | ACTGCCTAAAGAGGTATATGGCATGTATAAAGTGCTCCATAAATGGAGCTTATTGGGA | | |
| | GAGTATAAGTCACAGGCCATGCCCCGCAAGGGGATGCACGAAGACCCACCGCGAGCCA | | |
| | GGAAGGGAGCACGGGGCTCTCTGCTCTGGGACCGGCAGTGAGCCGGACATCTGGGTCC | | |
| | TCCCAAGCCGGGCGGGCTGCCCCAGGGAGGAAGGGAGGGGGGCGAGCCTGAGCGGGCA | | |
| | CCTCGGCCCGCAGGAGGTCTGCAGCGAGCTGTGGTGTCTGAGCAAGAGCAACCGGTGC | | |
| | ATCACCAACAGCATCCCGGCCGCCGAGGGCACGCTGTGCCAGACGCACACCATCGACA | | |
| | AGGGGTGGTGCTACAAACGGGTCTGTGTCCCCTTTGGGTCGCGCCCAGAGGGTGTGGA | | |
| | CGGAGCCTGGGGGCCGTGGACTCCATGGGGCGACTGCAGCCGGACCTGTGGCGGCGGC | | |
| | GTGTCCTCTTCTAGCCGTCACTGCGACAGCCCCAGGCCAACCATCGGGGGCAAGTACT | | |
| | GTCTGGGTGAGAGAAGGCGGCACCGCTCCTGCAACACGGATGACTGTCCCCCTCGCTC | | |
| | CCAGGACTTCAGAGAAGTGCAGTGTTCTGAATTTGACAGCATCCCTTTCCGTGGGAAA | | |
| | TTCTACAAGTGGAAAACGTACCGGGGAGGGGGCGTGAAGGCCTGCTCGCTCACGTGCC | | |
| | TAGCGGAAGGCTTCAACTTCTACACGGAGAGGGCGGCAGCCGTGGTGGACGGGACACC | | |
| | CTGCCGTCCAGACACGGTGGACATTTGCGTCAGTGGCGAATGCAAGCACGTGGGCTGC | | |
| | GACCGAGTCCTGGGCTCCGACCTGCGGGAGGACAAGTGCCGAGTGTGTGGCGGTGACG | | |
| | GCAGTGCCTGCGAGACCATCGAGGGCGTCTTCAGCCCAGCCTCACCTGGGGCCGGGTA | | |
| | CGAGGATGTCGTCTGGATTCCCAAAGGCTCCGTCCACATCTTCATCCAGGATCTGAAC | | |
| | CTCTCTCTCAGTCACTTGGCCCTGAAGGGAGACCAGGAGTCCCTGCTGCTGGAGGGGC | | |
| | TGCCCGGGACCCCCCAGCCCCACCGTCTGCCTCTAGCTGGGACCACCTTTCAACTGCG | | |
| | ACAGGGGCCAGACCAGGTCCAGAGCCTCGAAGCCCTGGGACCGATTAATGCATCTCTC | | |
| | ATCGTCATGGTGCTGGCCCGGACCGAGCTGCCTGCCCTCCGCTACCGCTTCAATGCCC | | |
| | CCATCGCCCGTGACTCGCTGCCCCCCTACTCCTGGCACTATGCGCCCTGGACCAAGTG | | |
| | CTCGGCCCAGTGTGCAGGCGGTAGCCAGGTGCAGGCGGTGGAGTGCCGCAACCAGCTG | | |
| | GACAGCTCCGCGGTCGCCCCCCACTACTGCAGTGCCCACAGCAAGCTGCCCAAAAGGC | | |
| | AGCGCGCCTGCAACACGGAGCCTTGCCCTCCAGACTGGGTTGTAGGGAACTGGTCGCT | | |
| | CTGCAGCCGCAGCTGCGATGCAGGCGTGCGCAGCCGCTCGGTCGTGTGCCAGCGCCGC | | |
| | GTCTCTGCCGCGGAGGAGAAGGCGCTGGACGACAGCGCATGCCCGCAGCCGCGCCCAC | | |
| | CTGTACTGGAGGCCTGCCACGGCCCCACTTGCCCTCCGGAGTGGGCGGCCCTCGACTG | | |
| | GTCTGAGTGCACCCCCAGCTGCGGGCCGGGCCTCCGCCACCGCGTGGTCCTTTGCAAG | | |
| | AGCGCAGACCACCGCGCCACGCTGCCCCCGGCGCACTGCTCACCCGCCGCCAAGCCAC | | |
| | CGGCCACCATGCGCTGCAACTTGCGCCGCTGCCCCCCGGCCCGCTGGGTGGCTGGCGA | | |
| | GTGGGGTGAGTGCTCTGCACAGTGCGGCGTCGGGCAGCGGCAGCGCTCGGTGCGCTGC | | |
| | ACCAGCCACACGGGCCAGGCGTCGCACGAGTGCACGGAGGCCCTGC | | |
| | ORF Start: at 1798 | | ORF Stop: at 3700 |
| | SEQ ID NO:392 | 634 aa | MW at 68754.0 kD |
| NOV52d, | YCLKRYMACIKCSINGAYWESISHRPCPARGCTKTHREPGREHGALCSGTGSEPDIWV | | |
| CG51213-03 | | | |
| Protein Sequence | LPSRAGCPREEGRGASLSGHLGPQEVCSELWCLSKSNRCITNSIPAAEGTLCQTHTID | | |
| | KGWCYKRVCVPFGSRPEGVDGAWGPWTPWGDCSRTCGGGVSSSSRHCDSPRPTIGGKY | | |
| | CLGERRRHRSCNTDDCPPGSQDFREVQCSEFDSIPFRGKFYKWKTYRGGGVKACSLTC | | |
| | LAEGFHFYTERAAAVVDGTPCRPDTVDICVSGECKIIVGCDRVLGSDLREDKCRVCGGD | | |
| | GSACETIEGVFSPASPGAGYEDVVWIPKGSVHIFIQDLNLSLSHLALKGDQESLLLEG | | |
| | LPGTPQPHRLPLAGTTFQLRQGPDQVQSLEALGPINASLIVMVLARTELPALRYRFNA | | |
| | PIARDSLPPYSWHYAPWTKCSAQCAGGSQVQAVECRNQLDSSAVAPHYCSAHSKLPKR | | |
| | QRACNTEPCPPDWVVGNWSLCSRSCDAGVRSRSVVCQRRVSAAEEKALDDSACPQPRP | | |
| | PVLEACHGPTCPPEWAALDWSECTPSCGPGLRHRVVLCKSADHRATLPPAHCSPAAKP | | |
| | PATMRCNLRRCPPARNVAGEWGECSAQCGVGQRQRSVRCTSHTGQASHECTEAL | | |
| | SEQ ID NO:393 | 2804 bp | |
| NOV52e, | TGGCCAGCCAGGCCTGAAGCGATCGGTCAGCCGAGAGCGCTACGTGGAGACCCTGGTG | | |
| CG51213-04 | | | |
| DNA Sequence | GTGGCTGACAAGATGATGGTGGCCTATCACGGGCGCCGGGATGTGGAGCAGTATGTCC | | |
| | TGGCCATCATGAACATTCAGGTTGCCAAACTTTTCCAGGACTCGAGTCTGGGAAGCAC | | |
| | CGTTAACATCCTCGTAACTCGCCTCATCCTGCTCACGCAGGACCAGCCCACTCTGGAG | | |
| | ATCACCCACCATGCCGGGAAGTCCCTGGACAGCTTCTGTAAGTGGCAGAAATCCATCG | | |
| | TGAACCACAGCGGCCATGGCAATGCCATTCCAGAGAACGGTGTGGCTAACCATGACAC | | |
| | AGCAGTGCTCATCACACGCTATGACATCTGCATCTACAAGAACAAACCCTGCGGCACA | | |
| | CTAGGCCTGGCCCCGGTGGGCGGAATGTGTGAGCGCGAGAGAAGCTGCAGCGTCAATG | | |
| | AGGACATTGGCCTGGCCACAGCGTTCACCATTGCCCACGAGATCGGGCACACATTCGG | | |
| | CATGAACCATGACGGCGTGGGAAACAGCTGTGGGGCCCGTGGTCAGGACCCAGCCAAG | | |
| | CTCATGGCTGCCCACATTACCATGAAGACCAACCCATTCGTGTGGTCATCCTGCAGCC | | |
| | GTGACTACATCACCAGCTTTCTAGACTCGGGCCTGGGGCTCTGCCTGAACAACCGGCC | | |
| | CCCCAGACAGGACTTTGTGTACCCGACAGTGGCACCGGGCCAAGCCTACGATGCAGAT | | |
| | GAGCAATGCCGCTTTCAGCATGGAGTCAAATCGCGTCAGTGTAAATACGGGGAGGTCT | | |
| | GCAGCGAGCTGTGGTGTCTGAGCAAGAGCAACCGGTGCATCACCAACAGCATCCCGGC | | |
| | CGCCGAGGGCACGCTGTGCCAGACGCACACCATCGACAAGGGGTGGTGCTACAAACGG | | |
| | GTCTGTGTCCCCTTTGGGTCGCGCCCAGAGGGTGTGGACGGAGCCTGGGGGCCGTGGA | | |
| | CTCCATGGGGCGACTGCAGCCGGACCTGTGGCGGCGGCGTGTCCTCTTCTAGCCGTCA | | |
| | CTGCGACAGCCCCAGGCCAACCATCGGGGGCAAGTACTGTCTGAGTGAGAGAAGGCGG | | |
| | CACCGCTCCTGCAACACGGATGACTGTCCCCCTGGCTCCCAGGACTTCAGAGAAGTGC | | |
| | AGTGTTCTGAATTTGACAGCATCCCTTTCCGTGGGAAATTCTACAAGTGGAAAACGTA | | |
| | CCGGGGAGGGGGCGTGAAGGCCTGCTCGCTCACGAGCCTAGCGGAAGGCTTCAACTTC | | |
| | TACACGGAGAGGGCGGCAGCCGTGGTGGACGGGACACCCTGCCGTCCAGACACGGTGG | | |
| | ACATTTGCGTCAGTGGCGAATGCAAGCACGTGGGCTGCGACCGAGTCCTGGGCTCCGA | | |
| | CCTGCGGGAGGACAAGTGCCGAGTGTGTGGCGGTGACGGCAGTGCCTGCGAGACCATC | | |
| | GAGGGCGTCTTCAGCCCAGCCTCACCTGGGGCCGGGTACGAGGATGTCGTCTGGATTC | | |
| | CCAAAGGCTCCGTCCACATCTTCATCCAGGATCTGAACCTCTCTCTCAGTCACTTGGC | | |
| | CCTGAAGGGAGACCAGGAGTCCCTGCTGCTGGAGGGGCTGCCTGGGACCCCCCAGCCC | | |
| | CACCGTCTGCCTCTAGCTGGGACCACCTTTCAACTGCGACAGGGGCCAGACCAGGTCC | | |
| | AGAGCCTCGAAGCCCTGGGACCGATTAATGCATCTCTCATCGTCATGGTGCTGGCCCG | | |
| | GACCGAGCTGCCTGCCCTCCGCTACCGCTTCAATGCCCCCATCGCCCGTGACTCGCTG | | |
| | CCCCCCTACTCCTGGCACTATGCGCCCTGGACCAAGTGCTCGGCCCAGTGTGCAGGCG | | |
| | GTAGCCAGGTGCAGGCGGTGGAGTGCCGCAACCAGCTGGACAGCTCCGCGGTCGCCCC | | |
| | CCACTACTGCAGTGCCCACAGCAAGCTGCCCAAAAGGCAGCGCGCCTGCAACACGGAG | | |
| | CCTTGCCCTCCAGACTGGGTTGTAGGGAACTGGTCGCTCTGCAGCCGCAGCTGCGATG | | |
| | CAGGCGTGCGCAGTCGCTCGGTCGTGTGCCAGCGCCGCGTCTCTGCCGCGGAGGAGAA | | |
| | GGCGCTGGACGACAGCGCATGCCCGCAGCCGCGCCCACCTGTACTGGAGGCCTGCCAC | | |
| | GGCCCCACTTGCCCTCCGGAGTGGGCGGCCCTCGACTGGTCTGAGTGCACCCCCAGCT | | |
| | GCGGGCCGGGCCTCCGCCACCGCGTGGTCCTTTGCAAGAGCGCAGACCACCGCGCCAC | | |
| | GCTGCCCCCGGCGCACTGCTCACCCGCCGCCAAGCCACCGGCCACCATGCGCTGCAAC | | |
| | TTGCGCCGCTGCCCCCCGGCCCGCTGGGTGGCTGGCGAGTGGGGTGAGTGCTCTGCAC | | |
| | AGTGCGGCGTCGGGCAGCGGCAGCGCTCGGTGCGCTGCACCAGCCACACGGGCCAGGC | | |
| | GTCGCACGAGTGCACGGAGGCCCTGCGGCCGCCCACCACGCAGCAGTGTGAGGCCAAG | | |
| | TGCGACAGCCCAACCCCCGGGGACGGCCCTGAAGAGTGCAAGGATGTGAACAAGGTCG | | |
| | CCTACTGCCCCCTGGTGCTCAAATTTCAGTTCTGCAGCCGAGCCTACTTCCGCCAGAT | | |
| | GTGCTGCAAAACCTGCCAGGGCCACTAGGGGGCGCGCGGCACCCGGAGCCACAGCTGG | | |
| | CGGGGTCTCCGCCGCCAGCCCTGCAGCGGGCCGGCCAAAGGGGGCCCCGGGGGGGCGG | | |
| | GAACTGGGAGGGAAGGGTGAGACGGAGCCGGAAGTTATTTATTGGGAACCCCTGCAGG | | |
| | GCCCTGGCTGGGGGGATGGA | | |
| | ORF Start: ATG at 71 | | ORF Stop: TAG at 2636 |
| | SEQ ID NO:394 | 855 aa | MW at 93285.7 kD |
| NOV52e, | MNVAYHGRRDVEQYVLAIMNIQVAKLFQDSSLGSTVNILVTRLILLTEDQPTLEITHH | | |
| CG51213-04 | | | |
| Protein Sequence | AGKSLDSFCKWQKSIVNHSGHGMAIPENGVANHDTAVLITRYDICIYKNKPCGTLGLA | | |
| | PVGGMCERERSCSVNEDIGLATAFTIAHEIGHTFGMNHDGVGNSCGARGQDPAKLMAA | | |
| | HITMKTNPFVWSSCSRDYITSFLDSGLGLCLNNRPPRQDFVYPTVAPGQAYDADEQCR | | |
| | FQHGVKSRQCKYGEVCSELWCLSKSNRCITNSIPAAEGTLCQTHTIDKGWCYKRVCVP | | |
| | FGSRPEGVDGAWGPWTPWGDCSRTCGGGVSSSSRHCDSPRPTIGGKYCLSERRRHRSC | | |
| | NTDDCPPGSQDFREVQCSEFDSTPFRGKFYKWKTYRGGGVKACSLTSLAEGFNFYTER | | |
| | AAAVVDGTPCRPDTVDICVSGECKHVGCDRVLGSDLREDKCRVCGGDGSACETIEGVF | | |
| | SPASPGAGYEDVVWIPKGSVHIFIQDLNLSLSHLALKGDQESLLLEGLPGTPQPHRLP | | |
| | LAGTTFQLRQGPDQVQSLEALGPINASLIVMVLARTELPALRYRFNAPIARDSLPPYS | | |
| | WHYAPWTKCSAQCAGGSQVQAVECRNQLDSSAVAPHYCSAHSKLPKRQRACNTEPCPP | | |
| | DWVVGNWSLCSRSCDAGVRSRSVVCQRRVSAAEEKALDDSACPQPRPPVLEACHGPTC | | |
| | PPEWAALDWSECTPSCGPGLRHRVVLCKSADHRATLPPAHCSPAAKPPATMRCNLRRC | | |
| | PPARWVAGEWGECSAQCGVCQRQRSVRCTSHTGQASHECTEALRPPTTQQCEAKCDSP | | |
| | TPGDGPEECKDVNKVAYCPLVLKFQFCSRAYFRQMCCKTCQGH | | |
| | SEQ ID NO:395 | 3400 bp | |
| NOV52f, | CGGTCTCAAGATGAGTTCCTGTCCAGTCTGGAGAGCTATGAGATCGCCTTCCCCACCC | | |
| CG51213-05 | | | |
| DNA Sequence | GCGTGGACCACAACGGGGCACTGCTGGCCTTCTCGCCACCTCCTCCCCGGAGGCAGCG | | |
| | CCCCGGCACGGGGGCCACAGCCGAGTCCCGCCTCTTCTACAAAGTAGCCTCGCCCAGC | | |
| | ACCCACTTCCTGCTGAACCTGACCCGCAGCTCCCGTCTACTGGCAGGGCACGTCTCCG | | |
| | TGGAGTACTGGACACGGGAGGGCCTGGCCTGGCAGAGGGCGGCCCGGCCCCACTGCCT | | |
| | CTACGCTGGTCACCTGCAGGGCCAGGCCAGCAGCTCCCATGTGGCCATCAGCACCTGT | | |
| | GGAGGCCTGCACGGCCTGATCGTGGCAGACGAGGAAGAGTACCTGATTGAGCCCCTGC | | |
| | ACGGTGGGCCCAAGGGTTCTCGGAGCCCGGAGGAAAGTGGACCACATGTGGTGTACAA | | |
| | GCGTTCCTCTCTGCGTCACCCCCACCTGGACACAGCCTGTGGAGTGAGAGATGAGAAA | | |
| | CCGTGGAAAGGGCGGCCATGGTGGCTGCGGACCTTGAAGCCACCGCCTGCCAGACCCC | | |
| | TGGGGAATGAAACAGAGCGTGGCCAGCCAGGCCTGAAGCGATCGGTCAGCCGAGAGCG | | |
| | CTACGTGGAGACCCTGGTGGTGGCTGACAAGATGATGGTGGCCTATCACGGGCGCCGG | | |
| | GATGTGGAGCAGTATGTCCTGGCCATCATGAAACATTGTTGCCAACTTTTCCAGGACT | | |
| | CGAGTCTGGGAAGCACCGTTAACATCCTCGTAACTCGCCTCATCCTGCTCACGGAGGA | | |
| | CCAGCCCACTCTGGAGATCACCCACCATGCCGGGAAGTCCCTAGACAGCTTCTGTAAG | | |
| | TGGCAGAAATCCATCGTGAACCACAGCGGCCATGGCAATGCCATTCCAGAGAACGGTG | | |
| | TGGCTAACCATGACACAGCAGTGCTCATCACACGCTATGACATCTGCATCTACAAGAA | | |
| | CAAACCCTGCGGCACACTAGGCCTGGCCCCGGTGGGCGGAATGTGTGAGCGCGAGAGA | | |
| | AGCTGCAGCGTCAATGAGGACATTGGCCTGCCACAAGCGTTCACCATTGCCCACGAGA | | |
| | TCGGGCACACATTCGGCATGAACCATGACGGCGTGGGAAACAGCTGTGGGGCCCGTGG | | |
| | TCAGGACCCAGCCAAGCTCATGGCTGCCCACATTACCATGAAGACCAACCCATTCGTG | | |
| | TGGTCATCCTGCAACCGTGACTACATCACCAGCTTTCTAGACTCGGGCCTGGGGCTCT | | |
| | GCCTGAACAACCGGCCCCCCAGACAGGACTTTGTGTACCCGACAGTGGCACCGGGCCA | | |
| | AGCCTACGATGCAGATGAGCAATGCCGCTTTCAGCATGGAGTCAAATCGCGTCAGTGT | | |
| | AAATACGGGGAGGTCTGCAGCGAGCTGTGGTGTCTGAGCAAGAGCAACCGGTGCATCA | | |
| | CCAACAGCATCCCGGCCGCCGAGGGCACGCTGTGCCAGACGCACACCATCGACAAGGG | | |
| | GTGGTGCTACAAACGGGTCTGTGTCCCCTTTGGGTCGCGCCCAGAGGGTGTGGACGGA | | |
| | GCCTGGGGGCCGTGGACTCCATGGGGCGACTGCAGCCGGACCTGTGGCGGCGGCGTGT | | |
| | CCTCTTCTAGTCGTCACTGCGACAGCCCCAGGCCAACCATCGGGGGCAAGTACTGTCT | | |
| | CGGTGAGAGAAGGCGGCACCGCTCCTGCAACACGGATGACTGTCCCCCTGGCTCCCAG | | |
| | GACTTCAGAGAAGTGCAGTGTTCTGAATTTGACAGCATCCCTTTCCGTGGGAAATTCT | | |
| | ACAAGTGGAAAACGTACCGGGGAGGGGGCGTGAAGGCCTGCTCGCTCACGAGCCTAGC | | |
| | GGAAGGCTTCAACTTCTACACGGAGAGGGCGGCAGCCGTGGTGGACGGGACACCCTGC | | |
| | CGTCCAGACACGGTGGACATTTGCGTCAGTGGCGAATGCAAGCACGTGGGCTGCGACC | | |
| | GAGTCCTGGGCTCCGACCTGCGGGAGGACAAGTGCCGAGTGTGTGGCGGTGACGGCAG | | |
| | TGCCTGCGAGACCATCGAGGGCGTCTTCAGCCCAGCCTCACCTGGGGCCGGGTACGAG | | |
| | GATGTCGTCTGGATTCCCAAAGGCTCCGTCCACATCTTCATCCAGGATCTGAACCTCT | | |
| | CTCTCAGTCACTTGGCCCTGAAGGGAGACCAGGAGTCCCTGCTGCTGGAGGGGCTGCC | | |
| | TGGGACCCCCCACCCCCACCGTCTGCCTCTAGCTGGGACCACCTTTCAACTGCGACAG | | |
| | GGGCCAGACCAGGTCCAGAGCCTCGAAGCCCTGGCACCGATTAATGCATCTCTCATCG | | |
| | TCATGGTGCTGGCCCGGACCGAGCTGCCTGCCCTCCGCTACCGCTTCAATGCCCCCAT | | |
| | CGCCCGTGACTCGCTGCCCCCCTACTCCTGGCACTATGCGCCCTGGACCAAGTGCTCG | | |
| | GCCCAGTGTGCAGGCGGTAGCCAGGTGCAGGCGGTGGAGTGCCGCAACCAGCTGGACA | | |
| | GCTCCGCGGTCGCCCCCCACTACTGCAGTGCCCACAGCAAGCTGCCCAAAAGGCAGCG | | |
| | CGCCTGCAACACGGAGCCTTGCCCTCCAGACTGGGTTGTAGGGAACTGGTCGCTCTGC | | |
| | AGCCGCAGCTGCGATGCAGGCGTGCGCAGTCGCTCGGTCGTGTGCCAGCGCCGCGTCT | | |
| | CTGCCGCGGAGGAGAAGGCGCTGGACGACAGCGCATGCCCGCAGCCGCGCCCACCTGT | | |
| | ACTGGAGGCCTGCCACGGCCCCACTTGCCCTCCGGAGTGGGCGGCCCTCGACTGGTCT | | |
| | GAGTGCACCCCCAGCTGCGGGCCGGGCCTCCGCCACCGCGTGGTCCTTTGCAAGAGCG | | |
| | CAGACCACCGCGCCACGCTGCCCCCGGCGCACTGCTCACCCGCCGCCAAGCCACCGGC | | |
| | CACCATGCGCTGCAACTTGCGCCGCTGCCCCCCGGCCCGCTGGGTGGCTGGCGAGTGG | | |
| | GGTGAGTGCTCTGCACAGTGCGGCGTCGGGCAGCGGCAGCGCTCGGTGCGCTGCACCA | | |
| | GCCACACGGGCCAGGCGTCGCACGAGTGCACGGAGGCCCTGCGGCCGCCCACCACGCA | | |
| | GCAGTGTGAGGCCAAGTGCGACAGCCCAACCCCCGGGGACGGCCCTGAAGAGTGCAAG | | |
| | GATGTGAACAAGGTCGCCTACTGCCCCCTGGTGCTCAAATTTCAGTTCTGCAGCCGAG | | |
| | CCTACTTCCGCCAGATGTGCTGCAAAACCTGCCAGGGCCACTAGGGGGCGCGCGGCAC | | |
| | CCGGAGCCACAGCTGGCGGGGTCTCCGCCGCCAGCCCTGCAGCGGGCCGGCCAAAGGG | | |
| | GGCCCCGGGGGGGCGGGAACTGGGAGGGAAGGGTGAGACGGAGCCGGAAGTTATTTAT | | |
| | TGGGAACCCCTGCAGGGCCCTGGCTGGGGGGATGGA | | |
| | ORF Start: at 1 | | ORF Stop: TAG at 3232 |
| | SEQ ID NO:396 | 1077 aa | MW at 118071.4 kD |
| NOV52f, | RSQDEFLSSLESYEIAFPTRVDHNGALLAFSPPPPRRQRRGTGATAESRLFYKVASPS | | |
| CG51213-05 | | | |
| Protein Sequence | THFLLNLTRSSRLLAGHVSVEYWTREGLAWQRAARPHCLYAGHLQGQASSSHVATSTC | | |
| | GGLHGLIVADEEEYLIEPLHGGPKGSRSPEESGPHVVYKRSSLRHPHLDTACGVRDEK | | |
| | PWKGRPWWLRTLKPPPARPLGNETERGQPGLKRSVSRERYVETLVVADKMMVAYHGRR | | |
| | DVEQYVLAIMNIVAKLFQDSSLGSTVNILVTRLILLTEDQPTLEITHHAGKSLDSFCK | | |
| | WQKSIVNHSGHGNAIPENGVANHDTAVLITRYDICIYKNKPCGTLGLAPVGGMCERER | | |
| | SCSVNEDIGLPQAFTIAHEIGHTFGMNHDGVGNSCGARGQDPAKLMAAHITMKTNPFV | | |
| | WSSCNRDYITSFLDSGLGLCLNNRPPRQDFVYPTVAPGQAYDADEQCRFQHGVKSRQC | | |
| | KYGEVCSELWCLSKSNRCITNSIPAAEGTLCQTHTIDKGWCYKRVCVPFGSRPEGVDG | | |
| | AWGPWTPWGDCSRTCGGGVSSSSRHCDSPRPTIGGKYCLGERRRHRSCNTDDCPPGSQ | | |
| | DFREVQCSEFDSIPFRGKFYKWKTYRGGGVKACSLTSLAEGFNFYTERAAAVVVGTPC | | |
| | RPDTVDICVSGECKHVGCDRVLGSDLREDKCRVCGGDGSACETIEGVFSPASPGAGYE | | |
| | DVVWIPKGSVHIFIQDLNLSLSHLALKGDQESLLLEGLPGTPQPHRLPLAGTTFQLRQ | | |
| | GPDQVQSLEALGPINASLIVMVLARTELPALRYRFNAPIARDSLPPYSWHYAPWTKCS | | |
| | AQCAGGSQVQAVECRNQLDSSAVAPHYCSAHSKLPKRQRACNTEPCPPDWVVGNWSLC | | |
| | SRSCDAGVRSRSVVCQRRVSAAEEKALDDSACPQPRPPVLEACHGPTCPPEWAALDWS | | |
| | ECTPSCGPGLRHRVVLCKSADHRATLPPAHCSPAAKPPATMRCNLRRCPPARWVAGEW | | |
| | GECSAQCGVGQRQRSVRCTSHTGQASHECTEALRPPTTQQCEAKCDSPTPGDGPEECK | | |
| | DVNKVAYCPLVLKFQFCSRAYFRQMCCKTCQGH | | |
| | SEQ ID NO:397 | | 978 bp |
| NOV52g, | TCCATAAATGGAGCTTATTGGGAGAGTATAAGTCACAGGCCATGCCCCGCAAGGGGAT | | |
| CG51213-06 | | | |
| DNA Sequence | GCACGAAGACCCACCGCGAGCCAGGAAGGGAGCACCGGGCTCTCTGCTCTGGGACCGG | | |
| | CAGTGAGCCGGACATCTGGGTCCTCCCAAGCCGGGCGGGCTGCCCCAGGGAGGAAGGG | | |
| | AGGGGGGCGAGCCTGAGCGGGCACCTCGGCCCGCAGGAGGTCTGCAGCGAGCTGTGGT | | |
| | GTCTGAGCAAGAGCAACCGGTGCATCACCAACAGCATCCCGGCCGCCGAGGGCACGCT | | |
| | GTGCCAGACGCACACCATCGACAAGGGGTGGTGCTACAAACGGGTCTGTGTCCCCTTT | | |
| | GGGTCGCGCCCAGAGGGTGTGGACGGAGCCTGGGGGCCGTGGACTCCATGGGGCGACT | | |
| | GCAGCCGGACCTGTGGCGGCGGCGTGTCCTCTTCTAGCCGTCACTGCGACAGCCCCAG | | |
| | GCCAACCATCGGGGGCAAGTACTGTCTGGGTGAGAGAAGGCGGCACCGCTCCTGCAAC | | |
| | ACGGATGACTGTCCCCCTGGCTCCCAGGACTTCAGAGAAGTGCAGTGTTCTGAATTTG | | |
| | ACAGCATCCCTTTCCGTGGGAAATTCTACAAGTGGAAAACGTACCGGAAAGGGGGCGT | | |
| | GAAGGCCTGCTCGCTCACGTGCCTAGCGCAAGGCTTCAACTTCTACACGGAGAGGGCG | | |
| | GCAGCCGTGGTGGACGGGACACCCTGCCGTCCAGACACGGTGGACATTTGCGTCAGTG | | |
| | GCGAATGCAAGCACGTGGGCTGCGACCGAGTCCTGGGCTCCGACCTGCGGGAGGACAA | | |
| | GTGCCGAGTGTGTGGCGGTGACGGCAGTGCCTGCGAGACCATCGAGGGCGTCTTCAGC | | |
| | CCAGCCTCACCTGGGGCCGGGTACGAGGATGTCGTCTGGATTCCCAAAGGCTCCGTCC | | |
| | ACATCTTCATCCAGGATCTGAACCTCTCTCTCAGTCACTTGGCCCTGAAG | | |
| | ORF Start: at 1 | | ORF Stop: end of |
| | | | sequence |
| | SEQ ID NO:398 | 1326 aa | MW at 35330.2 kD |

| | |
|---|---|
| NOV52g, | SINGAYWESISHRPCPARGCTKTHREPGREHRALCSGTGSEPDIWVLPSRAGCPREEG |
| CG51213-06 | |
| Protein Sequence | RGASLSGHLGPQEVCSELWCLSKSNRCITNSIPAAEGTLCQTHTIDKGWCYKRVCVPF |
| | GSRPEGVDGAWGPWTPWGDCSRTCGGGVSSSSRHCDSPRPTIGGKYCLGERRRHRSCN |
| | TDDCPPGSQDFREVQCSEFDSIPFRGKFYKWKTYRGGGVKACSLTCLAEGFNFYTERA |
| | AAVVDGTPCRPDTVDICVSGECKNVGCDRVLGSDLREDKCRVCGGDGSACETIEGVFS |
| | PASPGAGYEDVVWIPKGSVHIFIQDLNLSLSHLALK |

Sequence comparison of the above protein sequences yields the following sequence relationships shown in Table 52B.

TABLE 52B
Comparison of NOV52a against NOV52b through NOV52g.

| | | Identities/ |
|---|---|---|
| | | Similarities for |
| Protein | NOV52a Residues/ | the Matched |
| Sequence | Match Residues | Region |
| NOV52b | 54 . . . 465 | 412/412 (100%) |
| | 211 . . . 622 | 412/412 (100%) |
| NOV52c | 54 . . . 465 | 412/412 (100%) |
| | 223 . . . 634 | 412/412 (100%) |
| NOV52d | 54 . . . 465 | 412/412 (100%) |
| | 223 . . . 634 | 412/412 (100%) |
| NOV52e | 54 . . . 523 | 469/470 (99%) |
| | 386 . . . 855 | 469/470 (99%) |
| NOV52f | 54 . . . 523 | 469/470 (99%) |
| | 608 . . . 1077 | 469/470 (99%) |
| NOV52g | 54 . . . 169 | 116/116 (100%) |
| | 211 . . . 326 | 116/116 (100%) |

Further analysis of the NOV52a protein yielded the following properties shown in Table 52C.

TABLE 52C
Protein Sequence Properties NOV52a

| | |
|---|---|
| PSort | 0.6400 probability located in plasma membrane; 0.5231 |
| analysis: | probability located in outside; 0.1900 probability |
| | located in lysosome (lumen); 0.1000 probability |
| | located in endoplasmic reticulum (membrane) |
| SignalP | Cleavage site between residues 37 and 38 |
| analysis: | |

A search of the NOV52a protein against the Geneseq database, a proprietary database that contains sequences published in patents and patent publication, yielded several homologous proteins shown in Table 52D.

TABLE 52D
Geneseq Results for NOV52a

| | | | Identities/ | |
|---|---|---|---|---|
| | | | Similarities for | |
| Geneseq | Protein/Organism/Length | NOV52a Residues/ | the Matched | Expect |
| Identifier | [Patent #, Date] | Match Residues | Region | Value |
| AAU01292 | Human Thrombospondin repeat | 1 . . . 523 | 523/523 (100%) | 0.0 |
| | domain protein 2, TSR2 - Homo | 1 . . . 523 | 523/523 (100%) | |
| | sapiens, 523 aa. [WO200123561- | | | |
| | A2, 05 APR. 2001] | | | |
| AAU97888 | Human aggrecanase protein #2 - | 54 . . . 523 | 470/470 (100%) | 0.0 |
| | Homo sapiens, 1104 aa. | 634 . . . 1103 | 470/470 (100%) | |
| | [WO200234895-A2, 02 MAY | | | |
| | 2002] | | | |
| AAU72890 | Human metalloprotease partial | 54 . . . 523 | 470/470 (100%) | 0.0 |
| | protein sequence #2 - Homo | 634 . . . 1103 | 470/470 (100%) | |
| | sapiens, 1103 aa. [WO200183782- | | | |
| | A2, 08 NOV. 2001] | | | |
| AAB74945 | Human ADAM type metal protease | 54 . . . 523 | 470/470 (100%) | 0.0 |
| | MDTS2 protein SEQ ID NO: 10 - | 634 . . . 1103 | 470/470 (100%) | |
| | Homo sapiens, 1103 aa. | | | |
| | [JP2001008687-A, 16 JAN. 2001] | | | |
| AAB72300 | Human ADAMTS-10 alternative | 54 . . . 523 | 469/470 (99%) | 0.0 |
| | amino acid sequence - Homo | 603 . . . 1072 | 469/470 (99%) | |
| | sapiens, 1072 aa. [WO200111074- | | | |
| | A2, 15 FEB. 2001] | | | |

In a BLAST search of public sequence datbases, the NOV52a protein was found to have homology to the proteins shown in the BLASTP data in Table 52E.

TABLE 52E
Public BLASTP Results for NOV52a

| | | | Identities/ | |
|---|---|---|---|---|
| Protein | | | Similarities for | |
| Accession | | NOV52a Residues/ | the Matched | Expect |
| Number | Protein/Organism/Length | Match Residues | Portion | Value |
| CAC37778 | Sequence 3 from Patent | 1 . . . 523 | 523/523 (100%) | 0.0 |
| | WO0123561 - Homo sapiens | 1 . . . 523 | 523/523 (100%) | |
| | (Human), 523 aa. | | | |
| Q9H324 | ADAMTS-10 precursor (EC 3.4.24.-) | 54 . . . 523 | 469/470 (99%) | 0.0 |
| | (A disintegrin and | 608 . . . 1077 | 469/470 (99%) | |
| | metalloproteinase with | | | |
| | thrombospondin motifs 10) | | | |
| | (ADAM-TS 10) (ADAM-TS10) - | | | |
| | Homo sapiens (Human), 1077 aa | | | |
| | (fragment). | | | |
| P58459 | ADAMTS-10 (EC 3.4.24.-) (A | 75 . . . 522 | 416/449 (92%) | 0.0 |
| | disintegrin and metalloproteinase | 1 . . . 449 | 424/449 (93%) | |
| | with thrombospondin motifs 10) | | | |
| | (ADAM-TS 10) (ADAM-TS10) - | | | |
| | Mus musculus (Mouse), 450 aa | | | |
| | (fragment). | | | |
| CAC37777 | Sequence 1 from Patent | 54 . . . 465 | 412/412 (100%) | 0.0 |
| | WO0123561 - Homo sapiens | 223 . . . 634 | 412/412 (100%) | |
| | (Human), 634 aa (fragment). | | | |
| CAD20434 | Sequence 8 from Patent | 54 . . . 464 | 411/411 (100%) | 0.0 |
| | WO0188156 - Homo sapiens | 634 . . . 1044 | 411/411 (100%) | |
| | (Human), 1044 aa (fragment). | | | |

PFam analysis predicts that the NOV52a protein contains the domains shown in the Table 52F.

TABLE 52F
Domain Analysis of NOV52a

| | | Identities/ | |
|---|---|---|---|
| | | Similarities | |
| Pfam | NOV52a | for the Matched | Expect |
| Domain | Match Region | Region | Value |
| tsp_1 | 249 . . . 304 | 11/60 (18%) | 0.043 |
| | | 38/60 (63%) | |
| tsp_1 | 308 . . . 364 | 14/64 (22%) | 0.1 |
| | | 38/64 (59%) | |
| tsp_1 | 366 . . . 422 | 16/58 (28%) | 0.4 |
| | | 34/58 (59%) | |
| tsp_1 | 427 . . . 477 | 17/56 (30%) | 0.073 |
| | | 32/56 (57%) | |

### Example 53

The NOV53 clone was analyzed, and the nucleotide and encoded polypeptide sequences are shown in Table 53A.

TABLE 53A
NOV53 Sequence Analysis

| | SEQ ID NO:399 | 2245 bp | |
|---|---|---|---|
| NOV53a, | AGAACAGCTTGAAGACCGTTCATTTTTAAGTGACAAGAGACTCACCTCCAAGAAGCAA | | |
| CG56155-01 | | | |
| DNA Sequence | TTGTGTTTTCAGAATGATTTTATTCAAGCAAGCAACTTATTTCATTTCCTTGTTTGCT | | |
| | ACAGTTTCCTGTGGATGTCTGACTCAACTCTATGAAAACGCCTTCTTCAGAGGTGGGG | | |
| | ATGTAGCTTCCATGTACACCCCAAATGCCCAATACTGCCAOATGAGGTGCACATTCCA | | |
| | CCCAAGGTGTTTGCTATTCAGTTTTCTTCCAGCAAGTTCAATCAATGACATGGAGAAA | | |
| | AGGTTTGGTTGCTTCTTGAAAGATAGTGTTACAGGAACCCTGCCAAAAGTACATCGAA | | |
| | CAGGTGCAGTTTCTGGACATTCCTTGAAGCAATGTGGTCATCAAATAAGTGCTTGCCA | | |
| | TCGAGACATTTATAAAGGAGTTGATATGAGAGGAGTCAATTTTAATGTGTCTAAGGTT | | |
| | AGCAGTGTTGAAGAATGCCAAAAAAGGTGCACCAATAACATTCGCTGCCAGTTTTTTT | | |
| | CATATGCCACGCAAACATTTCACAAGGCAGAGTACCGGAACAATTGCCTATTAAAGTA | | |
| | CAGTCCCGGAGGAACACCTACCGCTATAAAGGTGCTGAGTAACGTGGAATCTGGATTC | | |
| | TCACTGAAGCCCTGTGCCCTTTCAGAAATTGGTTGCCACATGAACATCTTCCAGCATC | | |
| | TTGCGTTCTCAGATGTGGATGTTGCCAGGGTTCTCACTCCAGATGCTTTTGTGTGTCG | | |
| | GACCATCTGCACCTATCACCCCAACTGCCTCTTCTTTACATTCTATACAAATGTATGG | | |
| | AAAATCGAGTCACAAAGAAATGTTTGTCTTCTTAAAACATCTGAAAGTGGCACACCAA | | |
| | GTTCCTCTACTCCTCAAGAAAACACCATATCTGGATATAGCCTTTTAACCTGCAAAAG | | |
| | AACTTTACCTGAACCCTGCCATTCTAAAATTTACCCGGGAGTTGACTTTGGAGGAGAA | | |
| | GAATTGAATGTGACTTTTGTTAAAGGAGTGAATGTTTGCCAAGAGACTTGCACAAAGA | | |
| | TGATTCGCTGTCAGTTTTTCACTTATTCTTTACTCCCAGAAGACTGTAAGGAAGAGAA | | |
| | GTGTAAGTGTTTCTTAAGATTATCTATGGATGGTTCTCCAACTAGGATTGCGTATGGG | | |
| | ACACAAGGGAGCTCTGGTTACTCTTTGAGATTGTGTAACACTGGGGACAACTCTGTCT | | |
| | GCACAACAAAAACAAGCACACGCATTGTTGGAGGAACAAACTCTTCTTGGGGAGAGTG | | |
| | GCCCTGGCAGGTGAGCCTGCAGGTGAAGCTGACAGCTCAGAGGCACCTGTGTGGAGGG | | |
| | TCACTCATAGGACACCAGTGGGTCCTCACTGCTGCCCACTGCTTTGATGGGCTTCCCC | | |
| | TGCAGGATGTTTGGCGCATCTATAGTGGCATTTTAAATCTGTCAGACATTACAAAAGA | | |
| | TACACCTTTCTCACAAATAAAAGAGATTATTATTCACCAAAACTATAAAGTCTCAGAA | | |
| | GGGAATCATGATATCGCCTTGATAAAACTCCAGGCTCCTTTGAATTACACTGAATTCC | | |
| | AAAAACCAATATGCCTACCTTCCAAAGGTGACACAAGCACAATTTATACCAACTGTTG | | |
| | GGTAACCGGATGGGGCTTCTCGAAGGAGAAAGGTGAAATCCAAAATATTCTACAAAAG | | |
| | GTAAATATTCCTTTGGTAACAAATGAAGAATGCCAGAAAAGATATCAAGATTATAAAA | | |
| | TAACCCAACGGATGGTCTGTGCTGGCTATAAAGAAGGGGGAAAAGATGCTTGTAAGGG | | |
| | AGATTCAGGTCGTCCCTTAGTTTGCAAACACAACGGAATGTGGCGTTTGGTGGGCATC | | |
| | ACAAGCTGGGGTGAAGGCTGTGCCCGCAGGGAGCAACCTGGTGTCTACACCAAAGTCG | | |
| | CTGAGTACATGGACTGGATTTTAGAGAAAACACAGAGCAGTGATGGAAAAGCTCAGAT | | |
| | GCAGTCACCAGCATGAGAAGCAGTCCAGAGTCTAGGCAATTTTTACAACCTGAGTTCA | | |
| | AGTCAAATTCTGAGCCTGGGGGGTCCTCATCTGCAAAGCATGGAGAGTGGCATCTTCT | | |
| | TTGCATCCTAAGGACGAAAGACACAGTGCACTCAGAGCTGCTGAGGACAATGTCTGCT | | |
| | GAAGCCCCCTTTCAGCACGCCGTAACCAGGGGCTGACAATGCGAGGTCGCAACTGAGA | | |
| | TCTCCATGACTGTGTGTTGTGAAATAAAATGGTGAAAGATC | | |
| | ORF Start ATG at 72 | | ORF Stop: TGA at 1986 |
| | SEQ ID NO:400 | 638 aa | MW at 71369.0 kD |
| NOV53a, | MILFKQATYFISLFATVSCGCLTQLYENAFFRGGDVASMYTPNAQYCQMRCTFHPRCL | | |
| CG56155-01 | | | |
| Protein Sequence | LFSFLPASSINDMEKRFGCFLKDSVTGTLPKVHRTGAVSGHSLKQCGHQISACHRDIY | | |
| | KGVDMRGVNFNVSKVSSVEECQKRCTNNIRCQFFSYATQTFHKAEYRNNCLLKYSPGG | | |
| | TPTAIKVLSNVESGFSLKPCALSEIGCHMNIFQHLAFSDVDVARVLTPDAFVCRTICT | | |
| | YHPNCLFFTFYTNVWKIESQRNVCLLKTSESGTPSSSTPQENTISGYSLLTCKRTLPE | | |
| | PCHSKIYPGVDFGGEELNVTFVKGVNVCQETCTKMIRCQFFTYSLLPEDCKEEKCKCF | | |
| | LRLSMDGSPTRIAYGTQGSSGYSLRLCNTGDNSVCTTKTSTRIVGGTNSSWGEWPWQV | | |
| | SLQVKLTAQRHLCGGSLIGHQWVLTAAHCFDGLPLQDVWRIYSGILNLSDITKDTPFS | | |
| | QIKEIIIHQNYKVSEGNHDIALIKLQAPLNYTEFQKPICLPSKGDTSTIYTNCWVTGW | | |
| | CFSKEKGEIQNTLQKVNIPLVTNEECQKRYQDYKITQRMVCAGYKEGGKDACKGDSGG | | |
| | PLVCKHNGMWRLVGITSWGEGCARREQPGVYTKVAEYMDWILEKTQSSDGKAQMQSPA | | |
| | SEQ ID NO:401 | 2038 bp | |
| NOV53b, | GTTTTCAGAATGATTTTATTCAAGCAAGCAACTTATTTCATTTCCTTGTTTGCTACAG | | |
| CG56155-02 | | | |
| DNA Sequence | TTTCCTGTGGATGTCTGACTCAACTCTATGAAAACGCCTTCTTCAGAGGTGGGGATGT | | |
| | AGCTTCCATGTACACCCCAAATGCCCAATACTGCCAGATGAGGTGCACATTCCACCCA | | |
| | AGGTGTTTGCTATTCAGTTTTCTTCCAGCAAGTTCAATCAATGACATGGAGAAAAGGT | | |
| | TTGGTTGCTTCTTGAAAGATAGTGTTACAGGAACCCTGCCAAAAGTACATCGAACAGG | | |
| | TGCAGTTTCTGGACATTCCTTGAAGCAATGTGCTCATCAAATAAGTGCTTGCCATCGA | | |
| | GACATTTATAAAGCAGTTGATATGAGAGGAGTCAATTTTAATGTGTCTAAGGTTAGCA | | |
| | GTGTTGAAGAATGCCAAAAAAGGTGCACCAATAACATTCGCTGCCAGTTTTTTTCATA | | |
| | TGCCACGCAAACATTTCACAAGGCAGAGTACCGGAACAATTGCCTATTAAAGTACAGT | | |
| | CCCGGAGGAACACCTACCGCTATAAAGGTGCTGAGTAACGTGGAATCTGGATTCTCAC | | |
| | TGAAGCCCTGTGCCCTTTCAGAAATTGGTTGCCACATGAACATCTTCCAGCATCTTGC | | |
| | GTTCTCAGATGTGGATGTTGCCAGGTTTCTCACTCCAGATGCTTTTGTGTGTCGGACC | | |
| | ATCTGCACCTATCACCCCAACTGCCTCTTCTTTACATTCTATACAAATGTATGGAAAA | | |
| | TCGAGTCACAAAGAAATGTTTGTCTTCTTAAAACATCTGAAAGTGGCACACCAAGTTC | | |
| | CTCTACTCCTCAAGAAAACACCATATCTGGATATAGCCTTTTAACCTGCAAAAGAACT | | |
| | TTACCTGAACCCTGCCATTCTAAAATTTACCCGGGAGTTGACTTTGGAGGAGAAGAAT | | |
| | TGAATGTGACTTTTGTTAAAGGAGTGAATGTTTGCCAAGAGACTTGCACAAAGATGAT | | |
| | TCGCTGTCAGTTTTTCACTTATTCTTTACTCCCAGAAGACTGTAAGGAAGAGAAGTGT | | |
| | AAGTGTTTCTTAAGATTATCTATGGATGGTTCTCCAACTAGGATTGCGTATGGGACAC | | |
| | AAGGGAGCTCTGGTTACTCTTTGAGATTGTGTAACACTGGGGACAACGCTGTCTGCAC | | |
| | AACAAAAACAAGCACACGCATTGTTGGAGGAACAAACTCTTCTTGGGGAGAGTGGCCC | | |
| | TGGCAGGTGAGCCTGCAGGTGAAGCTGACAGCTCAGAGGCACCTGTGTGGAGGGTCAC | | |
| | TCATAGGACACCAGTGGGTCCTCACTGCTGCCCACTGCTTTGATGGGCTTCCCCTGCA | | |
| | GGATGTTTGGCGCATCTATAGTGGCATTTTAAATCTGTCAGACATTACAAAAGATACA | | |
| | CCTTTCTCACAAATAAAAGAGATTATTATTCACCAAAACTATAAAGTCTCAGAAGGGA | | |
| | ATCATGATATCGCCTTGATAAAACTCCAGGCTCCTTTGAATTACACTGAATTCCAAAA | | |
| | ACCAATATGCCTACCTTCCAAAGGTGACACAAGCACAATTTATACCAACTGTTGGGTA | | |
| | ACCGGATGGGGCTTCTCGAAGGAGAAAGGTGAAATCCAAAATATTCTACAAAAGGTAA | | |
| | ATATTCCTTTGGTAACAAATGAAGAATGCCAGAAAAGATATCAAGATTATAAAATAAC | | |
| | CCAACGGATGGTCTGTGCTGGCTATAAAGAAGGGGGAAAAGATGCTTGTAAGGGAGAT | | |
| | TCAGGTGGTCCCTTAGTTTGCAAACACAACGGAATGTGGCGTTTGGTGGGCATCACCA | | |
| | GCTGGGGTGAAGGCTGTGCCCGCAGGGAGCAACCTGGTGTCTACACCAAAGTCGCTGA | | |
| | GTACATGGACTGGATTTTAGAGAAAACACAGAGCAGTGATGGAAAAGCTCAGATGCAG | | |
| | TCACCAGCATGAGAAGCAGTCCAGAGTCTAGGCAATTTTTACAACCTGAGTTCAAGTC | | |
| | AAATTCTGAGCCTGGGGGGTCCTCATCTGCAAAGCATGAAGAGTGGCATCTTCTTTGC | | |
| | ATCCTAAG | | |
| | ORF Start: ATG at 10 | | ORF Stop: TGA at 1924 |
| | SEQ ID NO:402 | 638 aa | MW at 71401.1 kD |
| NOV53b, | MILFKQATYFISLFATVSCGCLTQLYENAFFRGGDVASMYTPNAQYCQMRCTFHPRCL | | |
| CG56155-02 | | | |
| Protein Sequence | LFSFLPASSINDMEKRFGCFLKDSVTGTLPKVHRTGAVSGHSLKQCGHQISACHRDIY | | |
| | KGVDMRGVNFNVSKVSSVEECQKRCTNNIRCQFFSYATQTFHKAEYRNNCLLKYSPGG | | |
| | TPTAIKVLSNVESGFSLKPCALSEIGCHMNIFQHLAFSDVDVARFLTPDAFVCRTICT | | |
| | YHPNCLFFTFYTNVWKIESQRNVCLLKTSESGTPSSSTPQENTISGYSLLTCKRTLPE | | |
| | PCHSKIYPGVDFGGEELNVTFVKGVNVCQETCTKMIRCQFFTYSLLPEDCKEEKCKCF | | |
| | LRLSMDGSPTRIAYGTQGSSGYSLRLCNTGDNAVCTTKTSTRIVGGTNSSWGEWPWQV | | |
| | SLQVKLTAQRHLCGGSLIGHQWVLTAAHCFDGLPLQDVWRIYSGILNLSDITKDTPFS | | |
| | QIKEIIIHQNYKVSEGNHDIALIKLQAPLNYTEFQKPICLPSKGDTSTIYTNCWVTGW | | |
| | GFSKEKGEIQNILQKVNIPLVTNEECQKRYQDYKITQRMVCAGYKEGGKDACKGDSGG | | |
| | PLVCKHNGMWRLVGITSWGEGCARREQPGVYTKVAEYMDWILEKTQSSDGKAQMQSPA | | |
| | SEQ ID NO:403 | 1869 bp | |
| NOV53c, | GGATCCGGATGTCTGACTCAACTCTATGAAAACGCCTTCTTCAGAGGTGGGGATGTAG | | |
| CG56155-03 | | | |
| DNA Sequence | CTTCCATGTACACCCCAAATGCCCAATACTGCCAGATGAGGTGCACATTCCACCCAAG | | |
| | GTGTTTGCTATTCAGTTTTCTTCCAGCAAGTTCAATCAATGACATGGAGAAAAGGTTT | | |
| | GGTTGCTTCTTGAAAGATAGTGTTACAGGAACCCTGCCAAAAGTACATCGAACAGGTG | | |
| | CAGTTTCTGGACATTCCTTGAAGCAATGTGGTCATCAAATAAGTGCTTGCCATCGAGA | | |
| | CATTTATAAAGGAGTTGATATGAGAGGAGTCAATTTTAATGTGTCTAAGGTTAGCAGT | | |
| | GTTGAAGAATGCCAAAAAAGGTGCACCAGTAACATTCGCTGCCAGTTTTTTTCATATG | | |
| | CCACGCAAACATTTCACAAGGCAGAGTACCGGAACAATTGCCTATTAAAGTACAGTCC | | |
| | CGGAGGAACACCTACCGCTATAAAGGTGCTGAGTAACGTGGAATCTGGATTCTCACTG | | |
| | AAGCCCTGTGCCCTTTCAGAAATTGGTTGCCACATGAACATCTTCCAGCATCTTGCGT | | |
| | TCTCACATGTGGATGTTGCCAGGTTTCTCACTCCAGATGCTTTTGTGTGTCGGACCAT | | |
| | CTGCACCTATCACCCCAACTGCCTCTTCTTTACATTCTATACAAATGTATGGAAAATC | | |
| | GAGTCACAAAGAAATGTTTGTCTTCTTAAAACATCTGAAAGTGGCACACCAAGTTCCT | | |
| | CTACTCCTCAAGAAAACACCATATCTGGATATAGCCTTTTAACCTGCAAAAGAACTTT | | |
| | ACCTGAACCCTGCCATTCTAAAATTTACCCGGGAGTTGACTTTGGAGGAGAAGAATTG | | |
| | AATGTGACTTTTGTTAAAGGAGTGAATGTTTGCCAAGAGACTTGCACAAAGATGATTC | | |
| | GCTGTCAGTTTTTCACTTATTCTTTACTCCCAGAAGACTGTAAGGAAGAGAAGTGTAA | | |
| | GTGTTTCTTAAGATTATCTATGGATGGTTCTCCAACTAGGATTGCGTATGGGACACAA | | |
| | GGGAGCTCTCGTTACTCTTTGAGATTGTGTAACACTGGGGACAACGCTGTCTGCACAA | | |
| | CAAAAACAAGCACACGCATTGTTGGAGGAACAAACTCTTCTTGGGGAGAGTGGCCCTG | | |
| | GCAGGTGAGCCTGCAGGTGAAGCTGACAGCTCAGAGGCACCTGTGTGGAGGGTCACTC | | |
| | ATAGGACACCAGTGGGTCCTCACTGCTGCCCACTGCTTTGATGGGCTTCCCCTGCAGG | | |
| | ATGTTTGGCGCATCTATAGTGGCATTTTAAATCTGTCAGACATTACAAAAGATACACC | | |
| | TTTCTCACAAATAAAAGAGATTATTATTCACCAAAACTATAAAGTCTCAGAAGGGAAT | | |
| | CATGATATCGCCTTGATAAAACTCCAGGCTCCTTTGAATTACACTGAATTCCAAAAAC | | |
| | CAATATGCCTACCTTCCAAAGGTGACACAAGCACAATTTATACCAACTGTTGGGTAAC | | |
| | CGGATGGGGCTTCTCGAAGGAGAAAGGTGAAATCCAAAATATTCTACAAAAGGTAAAT | | |
| | ATTCCTTTGGTAACAAATGAAGAATGCCAGAAAAGATATCAAGATTATAAAATAACCC | | |
| | AACGGATGGTCTGTGCTGGCTATAAAGAAGGGGGAAAAGATGCTTGTAAGGGAGATTC | | |
| | AGGTGGTCCCTTAGTTTGCAAACACAATGGAATGTGGCGTTTGGTGGGCATCACCAGC | | |
| | TGGGGTGAAGGCTGTGCCCGCAGGGAGCAACCTGGTGTCTACACCAAAGTCGCTGAGT | | |
| | ACATGGACTGGATTTTAGAGAAAACACAGAGCAGTGATGGAAAAGCTCAGATGCAGTC | | |
| | ACCAGCACTCGAG | | |
| | ORF Start: at 7 | | ORF Stop: at 1864 |
| | SEQ ID NO:404 | 619 aa | MW at 69208.4 kD |

| | |
|---|---|
| NOV53c, | GCLTQLYENAFFRGGDVASMYTPNAQYCQMRCTFHPRCLLFSFLPASSINDMEKRFGC |
| CG56155-03 | |
| Protein Sequence | FLKDSVTGTLPKVHRTGAVSGHSLKQCGHQISACHRDIYKGVDMRGVNFNVSKVSSVE |
| | ECQKRCTSNIRCQFFSYATQTFHKAEYRNNCLLKYSPGGTPTAIKVLSNVESGFSLKP |
| | CALSEIGCHMNIFQHLAFSDVDVARFLTPDAFVCRTICTYHPNCLFFTFYTNVWKIES |
| | QRNVCLLKTSESGTPSSSTPQENTISGYSLLTCKRTLPEPCHSKIYPGVDFGGEELNV |
| | TFVKGVNVCQETCTKMIRCQFFTYSLLPEDCKEEKCKCFLRLSMDGSPTRIAYGTQGS |
| | SGYSLRLCNTGDNAVCTTKTSTRIVGGTNSSWGEWPWQVSLQVKLTAQRHLCGGSLIG |
| | HQWVLTAAHCFDGLPLQDVWRIYSGILNLSDITKDTPFSQIKEIIIHQNYKVSEGNHD |
| | IALIKLQAPLNYTEFQKPICLPSKGDTSTIYTNCWVTGWGFSKEKGEIQNILQKVNIP |
| | LVTNEECQKRYQDYKITQRMVCAGYKEGGKDACKGDSGGPLVCKHNGMWRLVGITSWG |
| | EGCARREQPGVYTKVAEYMDWILEKTQSSDGKAQMQSPA |

Sequence comparison of the above protein sequences yields the following sequence relationships shown in Table 53B.

TABLE 53B
Comparison of NOV53a against NOV53b and NOV53c.

| | | Identities/ |
|---|---|---|
| | | Similarities for |
| Protein | NOV53a Residues/ | the Matched |
| Sequence | Match Residues | Region |
| NOV53b | 1 . . . 638 | 636/638 (99%) |
| | 1 . . . 638 | 637/638 (99%) |
| NOV53c | 20 . . . 638 | 616/619 (99%) |
| | 1 . . . 619 | 618/619 (99%) |

Further analysis of the NOV53a protein yielded the following properties shown in Table 53C.

TABLE 53C
Protein Sequence Properties NOV53a

| | |
|---|---|
| PSort | 0.3700 probability located in outside; 0.1900 |
| analysis: | probability located in lysosome (lumen); 0.1000 |
| | probability located in endoplasmic reticulum |
| | (membrane); 0.1000 probability located |
| | in endoplasmic reticulum (lumen) |
| SignalP | Cleavage site between residues 20 and 21 |
| analysis: | |

A search of the NOV53a protein against the Geneseq database, a proprietary database that contains sequences published in patents and patent publication, yielded several homologous proteins shown in Table 53D.

TABLE 53D
Geneseq Results for NOV53a

| | | | Identities/ | |
|---|---|---|---|---|
| | | | Similarities for | |
| Geneseq | Protein/Organism/Length | NOV53a Residues/ | the Matched | Expect |
| Identifier | [Patent #, Date] | Match Residues | Region | Value |
| AAU68928 | Human protease domian of | 427 . . . 584 | 158/158 (100%) | 1e−92 |
| | kallikrein I - Homo sapiens, 158 aa. | 1 . . . 158 | 158/158 (100%) | |
| | [US6294663-B1, 25 SEP. 2001] | | | |
| AAU82755 | Amino acid sequence of novel | 319 . . . 621 | 115/306 (37%) | 9e−57 |
| | human protease #54 - Homo | 513 . . . 797 | 172/306 (55%) | |
| | sapiens, 802 aa. [WO200200860- | | | |
| | A2, 03 JAN. 2002] | | | |
| AAB24052 | Human PRO618 protein sequence | 319 . . . 621 | 115/306 (37%) | 9e−57 |
| | SEQ ID NO: 24 - Homo sapiens, | 513 . . . 797 | 172/306 (55%) | |
| | 802 aa. [WO200053754-A1, 14 | | | |
| | SEP. 2000] | | | |
| AAB44266 | Human PRO618 (UNQ354) protein | 319 . . . 621 | 115/306 (37%) | 9e−57 |
| | sequence SEQ ID NO: 169 - Homo | 513 . . . 797 | 172/306 (55%) | |
| | sapiens, 802 aa. [WO200053756- | | | |
| | A2, 14 SEP. 2000] | | | |
| AAY41710 | Human PRO618 protein sequence - | 319 . . . 621 | 115/306 (37%) | 9e−57 |
| | Homo sapiens, 802 aa. | 513 . . . 797 | 172/306 (55%) | |
| | [WO9946281-A2, 16 SEP. 1999] | | | |

In a BLAST search of public sequence datbases, the NOV53a protein was found to have homology to the proteins shown in the BLASTP data in Table 53E.

TABLE 53E
Public BLASTP Results for NOV53a

| | | NOV53a | Identities/ | |
|---|---|---|---|---|
| Protein | | Residues/ | Similarities | |
| Accession | | Match | for the | Expect |
| Number | Protein/Organism/Length | Residues | Matched Portion | Value |
| P03952 | Plasma kallikrein precursor (EC | 1 . . . 638 | 638/638 (100%) | 0.0 |
| | 3.4.21.34) (Plasma prekallikrein) | 1 . . . 638 | 638/638 (100%) | |
| | (Kininogenin) (Fletcher factor) - | | | |
| | Homo sapiens (Human), 638 aa. | | | |
| O97506 | Kallikrein - Sus scrofa (Pig), 643 | 1 . . . 635 | 505/635 (79%) | 0.0 |
| | aa. | 9 . . . 643 | 569/635 (89%) | |
| Q8R0P5 | Kallikrein B, plasma 1 - Mus | 1 . . . 638 | 487/638 (76%) | 0.0 |
| | musculus (Mouse), 638 aa. | 1 . . . 638 | 555/638 (86%) | |
| P26262 | Plasma kallikrein precursor (EC | 1 . . . 638 | 486/638 (76%) | 0.0 |
| | 3.4.21.34) (Plasma prekallikrein) | 1 . . . 638 | 554/638 (86%) | |
| | (Kininogenin) (Fletcher factor) - | | | |
| | Mus musculus (Mouse), 638 aa. | | | |
| P14272 | Plasma kallikrein precursor (EC | 1 . . . 638 | 478/638 (74%) | 0.0 |
| | 3.4.21.34) (Plasma prekallikrein) | 1 . . . 638 | 550/638 (85%) | |
| | (Kininogenin) (Fletcher factor) - | | | |
| | Rattus norvegicus (Rat), 638 aa. | | | |

PFam analysis predicts that the NOV53a protein contains the domains shown in the Table 53F.

TABLE 53F
Domain Analysis of NOV53a

| | | Identities/ | |
|---|---|---|---|
| | | Similarities | |
| | NOV53a | for the | Expect |
| Pfam Domain | Match Region | Matched Region | Value |
| PAN | 21 . . . 104 | 19/112 (17%) | 6.8e−14 |
| | | 66/112 (59%) | |
| PAN | 111 . . . 194 | 24/111 (22%) | 5.4e−15 |
| | | 67/111 (60%) | |
| PAN | 201 . . . 284 | 21/111 (19%) | 1.3e−10 |
| | | 63/111 (57%) | |
| PAN | 292 . . . 375 | 23/111 (21%) | 2.3e−09 |
| | | 64/111 (58%) | |
| trypsin | 391 . . . 621 | 113/262 (43%) | 4.8e−100 |
| | | 196/262 (75%) | |

### Example 54

The NOV54 clone was analyzed, and the nucleotide and encoded polypeptide sequences are shown in Table 54A.

TABLE 54A
NOV54 Sequence Analysis

| | |
|---|---|
| | SEQ ID NO: 405 1010 bp |
| NOV54a, | CGTATTGCTCGGCCCGGGGAGTTTCGCCCCCTGCCCGGCTCCGCGGCGCGGAGGATGG |
| CG57191-01 | |
| DNA Sequence | CGTGGAAACGGCTGGGCGCGCTGGTGATGTTCCCTCTACAGATGATCTATCTGGTGGT |
| | GAAAGCAGCCGTCGGACTGGTGCTGCCCGCCAAGCTGCGGGACCTGTCGCGGGAGAAC |
| | GTCCTCATCACCGGCGGCGGGAGAGGCATCGGGCGTCAGCTCGCCCGCGAGTTCGCGG |
| | AGCGCGGCGCCAGAAAGATTGTTCTCTGGGGCCGGACTGAGAAATGCCTGAAGGAGAC |
| | GACGGAGGAGATCCGGCAGATGGGCACTGAGTGCCATTACTTCATCTGTGATGTGGGC |
| | AACCGGGAGGAGGTGTACCAGACGGCCAAGGCCGTCCGGGAGAAGGTGGGTGACATCA |
| | CCATCCTGGTGAACAATGCCGCCGTGGTCCATGGGAAGAGCCTAATGGACAGTGATGA |
| | TGATGCCCTCCTCAAGTCCCAACACATCAACACCCTGGGCCAGTTCTGGACCACCAAG |
| | GCCTTCCTGCCGCGTATGCTGGAGCTGCAGAATGGCCACATCGTGTGCCTCAACTCCG |
| | TGCTGGCACTGTCTGCCATCCCCGGTGCCATCGACTACTGCACATCCAAAGCGTCAGC |
| | CTTCGCCTTCATGGAGAGCCTGACCCTGGGGCTGCTGGACTGTCCGGGAGTCAGCGCC |
| | ACCACAGTGCTGCCCTTCCACACCAGCACCGAGATGTTCCAGGGCATGAGAGTCAGGT |
| | TTCCCAACCTCTTTCCCCCACTGAAGCCGGAGACGGTGGCCCGGAGGACAGTGGAAGC |
| | TGTGCAGCTCAACCAGGCCCTCCTCCTCCTCCCATGGACAATGCATGCCCTCGTTATC |
| | TTGAAAAGCATACTTCCACAGGCTGCACTCGAGGAGATCCACAAATTCTCAGGAACCT |
| | ACACCTGCATGAACACTTTCAAAGGGCGGACATGAAGACAGGATGAAGACATGCTTGA |
| | GGAGCCACGGAGTTTGGGGGCCAC |
| | ORF Start: ATG at 55 ORF Stop: TAG at 961 |
| | SEQ ID NO: 406 302 aa MW at 33520.0kD |
| NOV54a, | MAWKRLGALVMFPLQMIYLVVKAAVGLVLPAKLRDLSRENVLITGGGRGIGRQLAREF |
| CG57191-01 | |
| Protein Sequence | AERGARKIVLWGRTEKCLKETTEEIRQMGTECHYFICDVGNREEVYQTAKAVREKVGD |
| | ITILVNNAAVVHGKSLMDSDDDALLKSQHINTLGQFWTTKAFLPRMLELQNGHIVCLN |
| | SVLALSAIPGAIDYCTSKASAFAFMESLTLGLLDCPGVSATTVLPFHTSTEMFQGMRV |
| | RFPNLFPPLKPETVARRTVEAVQLNQALLLLPWTMHALVILKSILPQAALEEIHKFSG |
| | TYTCMNTFKGRT |
| | SEQ ID NO: 407 1330 bp |
| NOV54b, | GGAGTTTCGCCCCCTGCCCGGCTCCGCGGCGCGGAGGATGGTGTGGAAACGGCTGGGC |
| CG57191-03 | |
| DNA Sequence | GCGCTGGTGATGTTCCCTCTACAGATGATCTATCTGGTGGTGAAAGCAGCCGTCGGAC |
| | TGGTGCTGCCCGCCAAGCTGCGGGACCTGTCGCGGGAGAACGTCCCCATCACCGGCGG |
| | CGGGAGAGGCATCGGGCGTCAGCTCGCCCGCGAGTTCGCGGAGCGCGGCGCCAGAAAG |
| | ATTGTTCTCTGGGGCCGGACTGAGAAATGCCATTACTTCATCTGTGATGTGGGCAACC |
| | GGGAGGAGGTGTACCAGACGGCCAAGGCCGTCCGGGAGAAGGTGGGTGGCATCACCAT |
| | CCTGGTGAGCAATGCCGCCGTGGTCCATGGGAAGAGCCTAATGGACAGTGATGATGAT |
| | GCCTTCCTCAAGTCCCAACACATCAACACCCTGGGCCAGTTCTGGACCACCAAGGCCT |
| | TCCTGCCGCGTATGCTGGAGCTGCAGAATGGCCACATCGTGTGCCTCAACTCCGTGCT |
| | GGCACTGTCTGCCATCCCCGGTGCCATCGACTACTGCACATCCAAAGCGTCAGCCTTC |
| | CAGTGCTGCCCTTCCACACCAGCACCGAGATGTTCCAGGGCATGAGAGTCAGGTTTCC |
| | CAACCTCTTTCCCCCACTGAAGCCGGAGACGGTGGCCCGGAGGACAGTGGAAGCTGTG |
| | CAGCTCAACCAGGCCCTCCTCCTCCTCCCATGGACAATGCATGCCCTCGTTATCTTGA |
| | AAAGCATACTTCCACAGGCTGCACTCGAGGAGATCCACAAATTCTCAGGAACCTACAC |
| | CTGCATGAACACTTTCAAAGGGCGGACATAGAGACAGGATGAAGACATGCTTGAGGAG |
| | CCACGGAGTTTGGGGGCCACAGCACCTGGGCACACACCCGAGCACCTGTCCATTGGCA |
| | TGCTTCTGCTGGGTGAGCAGGACAGCTCCTGTCCCCAGCGAAGAATCCGGCTGCCCCT |
| | GGGCCAGTCCCAGGACCTTTGCACAGGACTGATGGGTATAACTGACCCCCACAGGGAG |
| | GCAGGAAAACAGCCAGAAGCCACCTTGACACTTTTGAACATTTCCAGTTCTGTAGAGT |
| | TTATTGTCAATTGCTTCTCAAGTCTAACCAGCCTCAGCAGTGTGCATAGACCATTTCC |
| | AGGAGGGTCTGTCCCCAGATGCTCTGCCTCCCGTTCCAAAACCCACTCATCCTCAGCT |
| | TGCACAAACTGGTTGAACGGCAGGAATGAAAAATAAAGAGAGATGGCTTTTGTG |
| | ORF Start: ATG at 38 ORF Stop: TAG at 899 |
| | SEQ ID NO: 408 287 aa MW at 31731.0kD |
| NOV54b, | MVWKRLGALVMFPLQMIYLVVKAAVGLVLPAKLRDLSRENVPITGGGRGIGRQLAREF |
| CG57191-03 | |
| Protein Sequence | AERGARKIVLWGRTEKCHYFICDVGNREEVYQTAKAVREKVGGITILVSNAAVVHGKS |
| | LMDSDDDAFLKSQHINTLGQFWTTKAFLPRMLELQNGHIVCLNSVLALSAIPGAIDYC |
| | TSKASAFAFMESLTLGLLDCPGVSATTVLPFHTSTEMFQGMRVRFPNLFPPLKPETVA |
| | RRTVEAVQLNQALLLLPWTMHALVILKSILPQAALEEIHKFSGTYTCMNTFKGRT |
| | SEQ ID NO: 409 992 bp |
| NOV54c, | GGAGTTTCGCCCCCTGCCCGGCTCCGCGGCGCGGAGGATGGTGTGGAAACGGCTGGGC |
| CG57191-02 | |
| DNA Sequence | GCGCTGGTGATGTTCCCTCTACAGATGATCTATCTGGTGGTGAAAGCAGCCGTCGGAC |
| | TGGTGCTGCCCGCCAAGCTGCGGGACCTGTCGCGGGAGAACGTCCTCATCACCGGCGG |
| | CGGGAGAGGCATCGGGCGTCAGCTCGCCCGCGAGTTCGCGGAGCGCGGCGCCAGAAAG |
| | ATTGTTCTCTGGGGCCGGACTGAGAAATGCCTGAAGGAGACGACAGAGGGGATCCGGC |
| | AGATGGGCACTGAGTGCCACTACTTCATCTGTGATGTGGGCAACCGGGAGGAGGTGTA |
| | CCAGACGGCCAAGGCCGTCCGGGAGAAGGTGGGTGACATCACCATCCTGGTGAACAAT |
| | GCCGCCGTGGTCCATGGGAAGAGCCTAATGGACAGTGATGATGATGCCCTCCTCAAGT |
| | CCCAACACATCAACACCCTGGGCCAGTTCTGGACCACCAAGGCCTTCCTGCCGCGTAT |
| | GCTGGAGCTGCAGAATGGCCACATCGTGTGCCTCAACTCCGTGCTGGCACTGTCTGCC |
| | ATCCCCGGTGCCATCGACTACTGCACATCCAAAGCGTCAGCCTTCGCCTTCATGGAGA |
| | GCCTGACCCTGGGGCTGCTGGACTGTCCGGGAGTCAGCGCCACCACAGTGCTGCCCTT |
| | CCACACCAGCACCGAGATGTTCCAGGGCATGAGAGTCAGGTTTCCCAACCTCTTTCCC |
| | CCCTCCTCCTCCTCCCATGGACAATGCATGCCCTCGTTATCTTGAAAAGCATACTTCC |
| | ACAGGCTGCACTCGAGGAGATCCACAAATTCTCAGGAACCTACACCTGCATGAACACT |
| | TTCAAAGGGCGGACATAGAGACAGGATGAAGACATGCTTGAGGAGCCACGGAGTTTGG |
| | GGGCCA |
| | ORF Start: ATG at 38 ORF Stop: TAG at 944 |
| | SEQ ID NO: 410 302 aa MW at 33476.0kD |
| NOV54c, | MVWKRLGALVMFPLQMIYLVVKAAVGLVLPAKLRDLSRENVLITGGGRGIGRQLAREF |
| CG57191-02 | |
| Protein Sequence | AERGARKIVLWGRTEKCLKETTEGIRQMGTECHYFICDVGNREEVYQTAKAVREKVGD |
| | ITILVNNAAVVHGKSLMDSDDDALLKSQHINTLGQFWTTKAFLPRMLELQNGHIVCLN |
| | SVLALSAIPGAIDYCTSKASAFAFMESLTLGLLDCPGVSATTVLPFHTSTEMFQGMRV |
| | RFPNLFPPLKPETVARRTVEAVQLNQALLLLPWTMHALVILKSILPQAALEEIHKFSG |
| | TYTCMNTFKGRT |

Sequence comparison of the above protein sequences yields the following sequence relationships shown in Table 54B.

TABLE 54B
Comparison of NOV54a against NOV54b and NOV54c.

| | | Identities/ |
|---|---|---|
| | | Similarities |
| Protein | NOV54a Residues/ | for the |
| Sequence | Match Residues | Matched Region |
| NOV54b | 1 . . . 302 | 282/302 (93%) |
| | 1 . . . 287 | 283/302 (93%) |
| NOV54c | 1 . . . 302 | 300/302 (99%) |
| | 1 . . . 302 | 300/302 (99%) |

Further analysis of the NOV54a protein yielded the following properties shown in Table 54C.

TABLE 54C
Protein Sequence Properties NOV54a

| | |
|---|---|
| PSort | 0.6850 probability located in endoplasmic |
| analysis: | reticulum (membrane); 0.6400 probability |
| | located in plasma membrane; 0.4600 |
| | probability located in Golgi body; 0.1000 |
| | probability located in endoplasmic reticulum |
| | (lumen) |
| SignalP | Cleavage site between residues 24 and 25 |
| analysis: | |

A search of the NOV54a protein against the Geneseq database, a proprietary database that contains sequences published in patents and patent publication, yielded several homologous proteins shown in Table 54D.

TABLE 54D
Geneseq Results for NOV54a

| | | NOV54a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAY92510 | Human OXRE-7 - Homo sapiens, | 1 . . . 302 | 301/302 (99%) | e−173 |
| | 302 aa. [WO200020604-A2, 13 | 1 . . . 302 | 301/302 (99%) | |
| | APR. 2000] | | | |
| AAW89191 | Bone morphogenetic protein | 1 . . . 195 | 177/196 (90%) | 2e−97 |
| | upregulated gene (29A) product - | 1 . . . 196 | 184/196 (93%) | |
| | Mus sp, 202 aa. [EP890639-A2, 13 | | | |
| | JAN. 1999] | | | |
| AAO05702 | Human polypeptide SEQ ID NO | 144 . . . 281 | 137/138 (99%) | 3e−74 |
| | 19594 - Homo sapiens, 138 aa. | 1 . . . 138 | 137/138 (99%) | |
| | [WO200164835-A2, 07 SEP. 2001] | | | |
| AAY97999 | Human SCAD family molecule | 9 . . . 298 | 105/293 (35%) | 2e−47 |
| | HSFM-1, SEQ ID NO: 1 - Homo | 11 . . . 302 | 167/293 (56%) | |
| | sapiens, 309 aa. [US6057140-A, | | | |
| | 02 MAY 2000] | | | |
| ABB72322 | Rat protein isolated from skin cells | 6 . . . 301 | 99/299 (33%) | 3e−46 |
| | SEQ ID NO: 646 - Rattus sp. 298 aa. | 5 . . . 298 | 170/299 (56%) | |
| | [WO200190357-A1, 29 NOV. | | | |
| | 2001] | | | |

In a BLAST search of public sequence datbases, the NOV54a protein was found to have homology to the proteins shown in the BLASTP data in Table 54E.

TABLE 54E
Public BLASTP Results for NOV54a

| | | NOV54a | Identities/ | |
|---|---|---|---|---|
| Protein | | Residues/ | Similarities | |
| Accession | | Match | for the | Expect |
| Number | Protein/Organism/Length | Residues | Matched Portion | Value |
| O75911 | Retinal short-chain | 1 . . . 302 | 302/302 (100%) | e−173 |
| | dehydrogenase/reductase | 1 . . . 302 | 302/302 (100%) | |
| | RETSDR1 (EC 1.-.-.-) - Homo | | | |
| | sapiens (Human), 302 aa. | | | |
| Q9BUC8 | Short-chain | 1 . . . 302 | 301/302 (99%) | e−173 |
| | dehydrogenase/reductase 1 - Homo | 1 . . . 302 | 301/302 (99%) | |
| | sapiens (Human), 302 aa. | | | |
| O77769 | Retinal short-chain | 1 . . . 302 | 297/302 (98%) | e−171 |
| | dehydrogenase/reductase | 1 . . . 302 | 300/302 (98%) | |
| | RETSDR1 (EC 1.-.-.-) - Bos taurus | | | |
| | (Bovine), 302 aa. | | | |
| Q91WR0 | Retinal short-chain | 1 . . . 302 | 286/302 (94%) | e−165 |
| | dehydrogenase/reductase 1 - Mus | 1 . . . 302 | 294/302 (96%) | |
| | musculus (Mouse), 302 aa. | | | |
| Q91XC3 | Similar to retinal short-chain | 1 . . . 302 | 285/302 (94%) | e−165 |
| | dehydrogenase/reductase 1 - Mus | 1 . . . 302 | 293/302 (96%) | |
| | musculus (Mouse), 302 aa. | | | |

PFam analysis predicts that the NOV54a protein contains the domains shown in the Table 54F.

TABLE 54F
Domain Analysis of NOV54a

| | | Identities/ | |
|---|---|---|---|
| | | Similarities | |
| | NOV54a | for the | Expect |
| Pfam Domain | Match Region | Matched Region | Value |
| adh_short | 37 . . . 292 | 67/284 (24%) | 1.1e−25 |
| | | 171/284 (60%) | |

### Example 55

The NOV55 clone was analyzed, and the nucleotide and encoded polypeptide sequences are shown in Table 55A.

TABLE 55A
NOV55 Sequence Analysis

| | |
|---|---|
| | SEQ ID NO: 411 1192 bp |
| NOV55a, | CGGCGACTGACCGTGGTCGTGGGCGGACGGCGGCTTGCAGCGTGGAGGAGCTGGGGTC |
| CG59595-01 | |
| DNA Sequence | GCTGTGGGTCGCGAAGCAGAGCCCGGGACGTGCGCGCTTGGTGCACGATCCTGAAGGG |
| | GAGCTCCGAGGGGCCCGGGTCGCCAGGGCTGCTGCGGCCATTCCCGGAGCCCGGCGCG |
| | GGGCCCGCGAGATACTGGTTTAGGCCGTCCCAGGGCTCCGGGCGCACCCGGTGGCCGC |
| | TGCTGCAGCGGAGGGAGCGCGGCGGCGCGGGGGCTCGGAGACAGCGTTTCTCCCGGAA |
| | GTCTTCCTCGGGCAGCAGGTGGGAAGTGGGAGCCGGAGCGGCAGCTGGCAGCGTTCTC |
| | TCCGCAGGTCGGCACCATGCGCCCTGCAGCCCTGCGCGGGGCCCTGCTGGGCTGCCTC |
| | TGCCTGGCGTTGCTTTGCCTGGGCGGTGCGGACAAGCGCCTGCGTGACAACCATGAGT |
| | GGAAAAAACTAATTATGGTTCAGCACTGGCCTGAGACAGTATGCGAGAAAATTCAAAA |
| | CGACTGTAGAGACCCTCCGGATTACTGGACAATACATGGACTATGGCCCGATAAAAGT |
| | GAAGGATGTAATAGATCGTGGCCCTTCAATTTAGAAGAGATTAAGGATCTTTTGCCAG |
| | AAATGAGGGCATACTGGCCTGACGTAATTCACTCGTTTCCCAATCGCAGCCGCTTCTG |
| | GAAGCATGAGTGGGAAAAGCATGGGACCTGCGCCGCCCAGGTGGATGCGCTCAACTCC |
| | CAGAAGAAGTACTTTGGCAGAAGCCTGGAACTCTACAGGGAGCTGGACCTCAACAGTG |
| | TGCTTCTAAAATTGGGGATAAAACCATCCATCAATTACTACCAAGTTGCAGATTTTAA |
| | AGATGCCCTTGCCAGAGTATATGGAGTGATACCCAAAATCCAGTGCCTTCCACCAAGC |
| | CAGGATGAGGAAGTACAGACAATTGGTCAGATAGAACTGTGCCTCACTAAGCAAGACC |
| | AGCAGCTGCAAAACTGCACCGAGCCGGGGGAGCAGCCGTCCCCCAAGCAGGAAGTCTG |
| | GCTGGCAAATGGGGCCGCCGAGAGCCGGGGTCTGAGAGTCTGTGAAGATGGCCCAGTC |
| | TTCTATCCCCCACCTAAAAAGACCAAGCATTGATGCCCAACTTTTGGAAATATTCTGT |
| | TTTAAAAAGCAAGAGAAATTCACAAACTGCAG |
| | ORF Start: ATG at 365 ORF Stop: TGA at 1133 |
| | SEQ ID NO: 412 1256 aa MW at 29480.5kD |
| NOV55a, | MRPAALRGALLGCLCLALLCLGGADKRLRDNHEWKKLIMVQHWPETVCEKIQNDCRDP |
| CG59595-01 | |
| Protein Sequence | PDYWTIHGLWPDKSEGCNRSWPFNLEEIKDLLPEMRAYWPDVINSFPNRSRFWKHEWE |
| | KHGTCAAQVDALNSQKKYFGRSLELYRELDLNSVLLKLGIKPSINYYQVADFKDALAR |
| | VYGVIPKIQCLPPSQDEEVQTIGQIELCLTKQDQQLQNCTEPGEQPSPKQEVWLANGA |
| | AESRGLRVCEDGPVFYPPPKKTKH |
| | SEQ ID NO: 413 708 bp |
| NOV55b, | GGATCCGACAAGCGCCTGCGTGACAACCATGAGTGGAAAAAACTAATTATGGTTCAGC |
| 169728691 | |
| DNA Sequence | ACTGGCCTGAGACAGTATGCGAGAAAATTCAAAACGACTGTAGAGACCCTCCGGATTA |
| | CTGGACAATACATGGACTATGGCCCGATAAAAGTGAAGGATGTAATAGATCGTGGCCC |
| | TTCAATTTAGAAGAGATTAAGGATCTTTTGCCAGAAATGAGGGCATACTGGCCTGACG |
| | TAATTCACTCGTTTCCCAATCGCAGCCGCTTCTGGAAGCATGAGTGGGAAAAGCATGG |
| | GACCTGCGCCGCCCAGGTGGATGCGCTCAACTCCCAGAAGAAGTACTTTGGCAGAAGC |
| | CTGGAACTCTACAGGGAGCTGGACCTCAACAGTGTGCTTCTAAAATTGGGGATAAAAC |
| | CATCCATCAATTACTACCAAGTTGCAGATTTTAAAGATGCCCTTGCCAGAGTATATGG |
| | AGTGATACCCAAAATCCAGTGCCTTCCACCAAGCCAGGATGAGGAAGTACAGACAATT |
| | GGTCAGATAGAACTGTGCCTCACTAAGCAAGACCAGCAGCTGCAAAACTGCACCGAGC |
| | CGGGGGAGCAGCCGTCCCCCAAGCAGGAAGTCTGGCTGGCAAATGGGGCCGCCGAGAG |
| | CCGGGGTCTGAGAGTCTGTGAAGATGCCCCAGTCTTCTATCCCCCACCTAAAAAGACC |
| | AAGCATCTCGAG |
| | ORF Start: at 1 ORF Stop: end of sequence |
| | SEQ ID NO: 414 236 aa MW at 27528.0kD |
| NOV55b, | GSDKRLRDNHEWKKLIMVQHWPETVCEKIQNDCRDPPDYWTIHGLWPDKSEGCNRSWP |
| 169728691 | |
| Protein Sequence | FNLEEIKDLLPEMRAYWPDVIHSFPNRSRFWKHEWEKHGTCAAQVDALNSQKKYFGRS |
| | LELYRELDLNSVLLKLGIKPSINYYQVADFKDALARVYGVIPKIQCLPPSQDEEVQTI |
| | GQIELCLTKQDQQLQNCTEPGEQPSPKQEVWLANGAAESRGLRVCEDGPVFYPPPKKT |
| | KHLE |
| | SEQ ID NO: 415 709 bp |
| NOV55c, | GGATCCGACAAGCGCCTGCGTGACAACCATGAGTGGAAAAGACTAATTATGGTTCAGC |
| 169728707 | |
| DNA Sequence | ACTGGCCTGAGACAGTATGCGAGAAAATTCAAAACGACTGTAGAGACCCTCCGGATTA |
| | CTGGACAATACATGGACTATGGCCCGATAAAAGTGAAGGATGTAATAGATCGTGGCCC |
| | TTCAATTTAGAAGAGATTAAGGGTCTTTTGCCAGAAATGAGGGCATACTGGCCTGACG |
| | TAATTCACTCGTTTCCCAATCGCAGCCGCTTCTGGAAGCATGAGTGGGAAAAGCATGG |
| | GACCGGCGCCGCCCAGGTGGATGCGCTCAACTCCCAGAAGAAGTACTTTGGCAGAAGC |
| | CTGGAACTCTACAGGGGGCTGGACCTCAACAGTGTGCTTCTAAAATTGGGGATAAAAC |
| | CATCCATCAATTACTACCAAGTTGCAGATTTTAAAGATGCCCTTGCCAGAGTATATGG |
| | AGTGATACCCAAAATCCAGTGCCTTCCACCAAGCCAGGATGAGGAAGTACAGACAATT |
| | GGTCAGATAGAACTGTGCCTCACTAAGCAAGACCAGCAGCTGCAAAACTGCACCGAGC |
| | CGGGGGAGCAGCCGTCCCCCAAGCAGGAAGTCTGGCTGGCAAATGGGCCCGCCGAGAG |
| | CCGGGGTCTGAGAGTCTGTGAAGATGGCCCAGTCTTCTATCCCCCACCTAAAAAGACC |
| | AAGCATCTCGAGA |
| | ORF Start: at 1 ORF Stop: end of sequence |
| | SEQ ID NO: 416 237 aa MW at 27379.8kD |
| NOV55c, | GSDKRLRDNHEWKRLIMVQHWPETVCEKIQNDCRDPPDYWTIHGLWPDKSEGCNRSWP |
| 169728707 | |
| Protein Sequence | FNLEEIKGLLPEMRAYWPDVIHSFPNRSRFWKHEWEKHGTGAAQVDALNSQKKYFGRS |
| | LELYRGLDLNSVLLKLGIKPSINYYQVADFKDALARVYGVIPKIQCLPPSQDEEVQTI |
| | GQIELCLTKQDQQLQNCTEPGEQPSPKQEVWLANGAAESRGLRVCEDGPVFYPPPKKT |
| | KHLEX |
| | SEQ ID NO: 417 708 bp |
| NOV55d, | GGATCCGACAAGCGCCTGCGTGACAACCATGAGTGGAAAAAACTAATTATGGTTCAGC |
| 169728746 | |
| DNA Sequence | ACTGGCCTGAGACAGTATGCGAGAAAATTCAAGACGACTGTAGAGACCCTCCGGATTA |
| | CTGGACAATACATGGACTATGGCCCGATAAAAGTGAAGGATGTAATAGATCGTGGCCC |
| | TTCAATTTAGAAGAGATTAAGGATCTTTTGCCAGAAATGAGGGCATACTGGCCTGACG |
| | TAATTCACTCGTTTCCCAATCGCAGCCGCTTCTGGAAGCATGAGTGGGAAAAGCATGG |
| | GACCTGCGCCGCCCAGGTGGATGCGCTCAACTCCCACAAGAAGTACTTTGGCAGAAGC |
| | CTGGAACTCTACAGGGAGCTGGACCTCAACAGTGTGCTTCTAAAATTGGGGATAAAAC |
| | CATCCATCAATTACTACCAAGTTGCGGATTTTAAAGATGCCCTTGCCAGAGTATATGG |
| | AGTGATACCCAAAATCCAGTGCCTTCCACCAAGCCGGGATGAGGAAGTACAGACAATT |
| | GGTCAGATAGAACTGTGCCTCACTAAGCAAGACCAGCAGCTGCAAAACTGCACCGAGC |
| | CGGGGGAGCAGCCGTCCCCCAAGCAGGAAGTCTGGCTGGCAAATGGGGCCGCCGAGAG |
| | CCGGGGTCTGAGAGTCTGTGAAGATGGCCCAGTCTTCTATCCCCCACCTAAAAAGACC |
| | AAGCATCTCGAG |
| | ORF Start: at 1 ORF Stop: end of sequence |
| | SEQ ID NO: 418 236 aa MW at 27557.0kD |
| NOV55d, | GSDKRLRDNHEWKKLIMVQHWPETVCEKIQDDCRDPPDYWTIHGLWPDKSEGCNRSWP |
| 169728746 | |
| Protein Sequence | FNLEEIKDLLPEMRAYWPDVIHSFPNRSRFWKHEWEKHGTCAAQVDALNSQKKYFGRS |
| | LELYRELDLNSVLLKLGIKPSINYYQVADFKDALARVYGVIPKIQCLPPSRDEEVQTI |
| | GQIELCLTKQDQQLQNCTEPGEQPSPKQEVWLANGAAESRGLRVCEDGPVFYPPPKKT |
| | SEQ ID NO: 419 708 bp |
| NOV55e, | GGATCCGACAAGCGCCTGCGTGACAACCATGAGTGGAAAAAACTAATTATGGTTCAGC |
| CG59595-02 | |
| DNA Sequence | ACTGGCCTGACACAGTATGCGAGAAAATTCAAAACGACTGTAGAGACCCTCCGGATTA |
| | CTGGACAATACATGGACTATGGCCCGATAAAAGTGAAGGATGTAATAGATCGTGGCCC |
| | TTCAATTTAGAAGAGATTAAGGATCTTTTGCCAGAAATGAGGGCATACTGGCCTGACG |
| | TAATTCACTCGTTTCCCAATCGCAGCCGCTTCTGGAAGCATGAGTGGGAAAAGCATGG |
| | GACCTGCGCCGCCCAGGTGGATGCGCTCAACTCCCAGAAGAAGTACTTTGGCAGAAGC |
| | CTGGAACTCTACAGGGAGCTGGACCTCAACAGTGTGCTTCTAAAATTGGGGATAAAAC |
| | CATCCATCAATTACTACCAAGTTGCAGATTTTAAAGATGCCCTTGCCAGAGTATATGG |
| | AGTGATACCCAAAATCCAGTGCCTTCCACCAAGCCAGGATGAGGAAGTACAGACAATT |
| | GGTCAGATAGAACTGTGCCTCACTAAGCAAGACCAGCAGCTGCAAAACTGCACCGAGC |
| | CGGGGGAGCAGCCGTCCCCCAAGCAGGAAGTCTGGCTGGCAAATGGGGCCGCCGAGAG |
| | CCGGGGTCTGAGAGTCTGTGAAGATGGCCCAGTCTTCTATCCCCCACCTAAAAAGACC |
| | AAGCATCTCGAG |
| | ORF Start: at 7 ORF Stop: at 703 |
| | SEQ ID NO: 420 232 aa MW at 27141.6kD |
| NOV55e, | DKRLRDNHEWKKLIMVQHWPETVCEKIQNDCRDPPDYWTIHGLWPDKSEGCNRSWPFN |
| CG59595-02 | |
| Protein Sequence | LEEIKDLLPEMRAYWPDVIHSFPNRSRFWKHEWEKHGTCAAQVDALNSQKKYFGRSLE |
| | LYRELDLNSVLLKLGIKPSINYYQVADFKDALARVYGVIPKIQCLPPSQDEEVQTIGQ |
| | IELCLTKQDQQLQNCTEPGEQPSPKQEVWLANGAAESRGLRVCEDGPVFYPPPKKTKH |
| | SEQ ID NO: 421 923 bp |
| NOV55f, | GAGACAGCGTTTCTCCCGGAAGTCTTCCTCGGGCAGCAGGTGGGAAGTGGGAGCCGGA |
| CG59595-03 | |
| DNA Sequence | GCGGCAGCTGGCAGCGTTCTCTCCGCAGGTCGGCACCATGCGCCCTGCAGCCCTGCGC |
| | GGGGCCCTGCTGGGCTGCCTCTGCCTGGCGTTGCTTTGCCTGGGCGGTGCGGACAAGC |
| | GCCTGCGTGACAACCATCAGTGGAAAAAACTAATTATGGTTCAGCACTGGCCTGAGAC |
| | AGTATGCGAGAAAATTCAAAACGACTGTAGAGACCCTCCGGATTACTGGACAATACAT |
| | GGACTATGGCCCGATAAAAGTGAAGGATGTAATAGATCGTGGCCCTTCAATTTAGAAG |
| | AGATTAAGGATCTTTTGCCAGAAATOAGGGCATACTGGCCTGACGTAATTCACTCGTT |
| | TCCCAATCGCAGCCGCTTCTGGAAGCATGAGTGGGAAAAGCATGGGACCTGCGCCGCC |
| | CAGGTGGATGCGCTCAACTCCCAGAAGAAGTACTTTGGCAGAAGCCTGGAACTCTACA |
| | GGGAGCTGGACCTCAACAGTGTGCTTCTAAAATTGGGGATAAAACCATCCATCAATTA |
| | CTACCAAGTTGCAGATTTTAAAGATGCCCTCGCCAGAGTATATGGAGTGATACCCAAA |
| | ATCCAGTGCCTTCCACCAAGCCAGGATGAGGAAGTACAGACAATTGGTCAGATAGAAC |
| | TGTGCCTCACTAAGCAAGACCAGCAGCTGCAAAACTGCACCGAGCCGGGGGAGCAGCC |
| | GTCCCCCAAGCAGGAAGTCTGGCTGGCAAATGGGGCCGCCGAGAGCCGGGGTCTGAGA |
| | GTCTGTGAAGATGGCCCAGTCTTCTATCCCCCACCTAAAAAGACCAAGCATTGATGCC |
| | CAAGTTTTGGAAATATTCTGTTTTAAAAAGCAAGAGAAATTCACAAACTGCAG |
| | ORF Start: ATG at 96 ORF Stop: TGA at 864 |
| | SEQ ID NO: 422 256 aa MW at 29480.5kD |
| NOV55f, | MRPAALRGALLGCLCLALLCLGGADKRLRDNHEWKKLIMVQHWPETVCEKIQNDCRDP |
| CG59595-03 | |
| Protein Sequence | PDYWTIHGLWPDKSEGCNRSWPFNLEEIKDLLPEMRAYWPDVIHSFPNRSRFWKHEWE |
| | KHGTCAAQVDALNSQKKYFGRSLELYRELDLNSVLLKLCIKPSINYYQVADFKDALAR |
| | VYGVIPKIQCLPPSQDEEVQTIGQIELCLTKQDQQLQNCTEPGEQPSPKQEVWLANGA |
| | AESRGLRVCEDGPVFYPPPKKTKH |
| | SEQ ID NO: 423 709 bp |
| NOV55g, | GGATCCGACAAGCGCCTGCGTGACAACCATGAGTGGAAAAGACTAATTATGGTTCAGC |
| CG59595-04 | |
| DNA Sequence | ACTGGCCTGAGACAGTATGCGAGAAAATTCAAAACGACTGTAGAGACCCTCCGGATTA |
| | CTGGACAATACATGGACTATGGCCCGATAAAAGTGAAGGATGTAATAGATCGTGGCCC |
| | TTCAATTTAGAAGAGATTAAGGGTCTTTTGCCAGAAATGAGGGCATACTGGCCTGACG |
| | TAATTCACTCGTTTCCCAATCGCAGCCGCTTCTGGAAGCATGAGTGGGAAAAGCATGG |
| | CACCGGCGCCGCCCAGGTGGATCCGCTCAACTCCCAGAAGAAGTACTTTGGCAGAAGC |
| | CTGGAACTCTACAGGGGGCTGGACCTCAACAGTGTGCTTCTAAAATTGGGGATAAAAC |
| | CATCCATCAATTACTACCAAGTTGCAGATTTTAAAGATGCCCTTGCCAGAGTATATGG |
| | AGTGATACCCAAAATCCAGTGCCTTCCACCAAGCCAGGATGAGGAAGTACAGACAATT |
| | GGTCAGATAGAACTGTGCCTCACTAAGCAAGACCAGCAGCTGCAAAACTGCACCGAGC |
| | CGGGGGAGCAGCCGTCCCCCAAGCAGGAAGTCTGGCTGGCAAATGGGGCCGCCGAGAG |
| | CCGGGGTCTGAGAGTCTGTGAAGATGGCCCAGTCTTCTATCCCCCACCTAAAAAGACC |
| | AAGCATCTCGAGA |
| | ORF Start: at 7 ORF Stop: at 703 |
| | SEQ ID NO: 424 232 aa MW at 26993.4kD |
| NOV55g, | DKRLRDNHEWKRLIMVQHWPETVCEKIQNDCRDPPDYWTIHGLWPDKSEGCNRSWPFN |
| CG59595-04 | |
| Protein Sequence | LEEIKGLLPEMRAYWPDVIHSFPNRSRFWKHEWEKHGTGAAQVDALNSQKKYFGRSLE |
| | LYRGLDLNSVLLKLGIKPSINYYQVADFKDALARVYGVIPKIQCLPPSQDEEVQTIGQ |
| | IELCLTKQDQQLQNCTEPGEQPSPKQEVWLANGAAESRGLRVCEDGPVFYPPPKKTKH |
| | SEQ ID NO: 425 708 bp |
| NOV55h, | GGATCCGACAAGCGCCTGCGTGACAACCATGAGTGGAAAAAACTAATTATGGTTCAGC |
| CG59595-05 | |
| DNA Sequence | ACTGGCCTGAGACAGTATGCGAGAAAATTCAAGACGACTGTAGAGACCCTCCGGATTA |
| | CTGGACAATACATGGACTATGGCCCGATAAAAGTGAAGGATGTAATAGATCGTGGCCC |
| | TTCAATTTAGAAGAGATTAAGGATCTTTTGCCACAAATGAGGGCATACTGGCCTGACG |
| | TAATTCACTCGTTTCCCAATCGCAGCCGCTTCTGGAAGCATGAGTGGGAAAAGCATGG |
| | GACCTGCGCCGCCCAGGTGGATGCGCTCAACTCCCAGAAGAAGTACTTTGGCAGAAGC |
| | CTGGAACTCTACAGGGAGCTGGACCTCAACAGTGTGCTTCTAAAATTGGGGATAAAAC |
| | CATCCATCAATTACTACCAAGTTCCGGATTTTAAAGATGCCCTTGCCAGAGTATATGG |
| | AGTGATACCCAAAATCCAGTGCCTTCCACCAAGCCGGGATGAGGAAGTACAGACAATT |
| | GGTCAGATAGAACTGTGCCTCACTAAGCAAGACCAGCAGCTGCAAAACTGCACCGAGC |
| | CGGGGGAGCAGCCGTCCCCCAAGCAGGAAGTCTGGCTGGCAAATGGGGCCGCCGAGAG |
| | CCGGGGTCTGAGAGTCTGTGAAGATGCCCCAGTCTTCTATCCCCCACCTAAAAAGACC |
| | AAGCATCTCGAG |
| | ORF Start: at 7 ORF Stop: at 703 |
| | SEQ ID NO: 426 232 aa MW at 27170.6kD |
| NOV55h, | DKRLRDNHEWKKLIMVQHWPETVCEKIQDDCRDPPDYWTIHGLWPDKSEGCNRSWPFN |
| CG59595-05 | |
| Protein Sequence | LEEIKDLLPEMRAYWPDVIHSFPNRSRFWKHEWEKHGTCAAQVDALNSQKKYFGRSLE |
| | LYRELDLNSVLLKLGIKPSINYYQVADFKDALARVYGVIPKIQCLPPSRDEEVQTIGQ |
| | IELCLTKQDQQLQ&CTEPGEQPSPKQEVWLANGAAESRGLRVCEDGPVFYPPPKKTKH |

Sequence comparison of the above protein sequences yields the following sequence relationships shown in Table 55B.

TABLE 55B
Comparison of NOV55a against NOV55b through NOV55h.

| | | Identities/ |
|---|---|---|
| | | Similarities |
| | NOV55a Residues/ | for the |
| Protein Sequence | Match Residues | Matched Region |
| NOV55b | 23 . . . 256 | 233/234 (99%) |
| | 1 . . . 234 | 234/234 (99%) |
| NOV55c | 23 . . . 256 | 229/234 (97%) |
| | 1 . . . 234 | 231/234 (97%) |
| NOV55d | 23 . . . 256 | 231/234 (98%) |
| | 1 . . . 234 | 234/234 (99%) |
| NOV55e | 25 . . . 256 | 232/232 (100%) |
| | 1 . . . 232 | 232/232 (100%) |
| NOV55f | 1 . . . 256 | 256/256 (100%) |
| | 1 . . . 256 | 256/256 (100%) |
| NOV55g | 25 . . . 256 | 228/232 (98%) |
| | 1 . . . 232 | 229/232 (98%) |
| NOV55h | 25 . . . 256 | 230/232 (99%) |
| | 1 . . . 232 | 232/232 (99%) |

Further analysis of the NOV55a protein yielded the following properties shown in Table 55C.

TABLE 55C
Protein Sequence Properties NOV55a

| | |
|---|---|
| PSort | 0.8200 probability located in outside; 0.1900 probability |
| analysis: | located in lysosome (lumen); 0.1000 probability located |
| | in endoplasmic reticulum (membrane); 0.1000 probability |
| | located in endoplasmic reticulum (lumen) |
| SignalP | Cleavage site between residues 25 and 26 |
| analysis: | |

A search of the NOV55a protein against the Geneseq database, a proprietary database that contains sequences published in patents and patent publication, yielded several homologous proteins shown in Table 55D.

TABLE 55D
Geneseq Results for NOV55a

| | | NOV55a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY21852 | Human signal peptide-contianing | 1 . . . 256 | 256/256 (100%) | e−158 |
| | protein (SIGP) (clone ID 2652271) - | 1 . . . 256 | 256/256 (100%) | |
| | Homo sapiens, 256 aa. | | | |
| | [WO9933981-A2, 08 JUL. 1999] | | | |
| AAW75103 | Human secreted protein encoded | 1 . . . 256 | 256/256 (100%) | e−158 |
| | by gene 47 clone HMCBP63 - | 1 . . . 256 | 256/256 (100%) | |
| | Homo sapiens, 256 aa. | | | |
| | [WO9839446-A2, 11 SEP. 1998] | | | |
| AAY48563 | Human breast tumour-associated | 1 . . . 256 | 255/256 (99%) | e−157 |
| | protein 24 - Homo sapiens, 284 aa. | 29 . . . 284 | 255/256 (99%) | |
| | [DE19813839-A1, 23 SEP. 1999] | | | |
| ABG12714 | Novel human diagnostic protein | 1 . . . 256 | 247/258 (95%) | e−150 |
| | #12705 - Homo sapiens, 342 aa. | 85 . . . 342 | 251/258 (96%) | |
| | [WO200175067-A2, 11 OCT. | | | |
| | 2001] | | | |
| ABG12711 | Novel human diagnostic protein | 49 . . . 256 | 184/208 (88%) | e−109 |
| | #12702 - Homo sapiens, 193 aa. | 1 . . . 193 | 187/208 (89%) | |
| | [WO200175067-A2, 11 OCT. | | | |
| | 2001] | | | |

In a BLAST search of public sequence datbases, the NOV55a protein was found to have homology to the proteins shown in the BLASTP data in Table 55E.

TABLE 55E
Public BLASTP Results for NOV55a

| | | NOV55a | Identities/ | |
|---|---|---|---|---|
| Protein | | Residues/ | Similarities | |
| Accession | | Match | for the | Expect |
| Number | Protein/Organism/Length | Residues | Matched Portion | Value |
| O00584 | Ribonuclease 6 precursor (EC | 1 . . . 256 | 256/256 (100%) | e−158 |
| | 3.1.27.-) - Homo sapiens | 1 . . . 256 | 256/256 (100%) | |
| | (Human), 256 aa. | | | |
| S78046 | ribonuclease 6 (EC 3.1.27.-) | 1 . . . 181 | 180/181 (99%) | e−109 |
| | precursor - human, 189 aa. | 1 . . . 181 | 180/181 (99%) | |
| Q9CQ01 | Ribonuclease 6 precursor (EC | 1 . . . 256 | 176/261 (67%) | e−105 |
| | 3.1.27.-) - Mus musculus | 1 . . . 259 | 207/261 (78%) | |
| | (Mouse), 259 aa. | | | |
| JE0172 | ribonuclease T2 (EC 3.1.27.1) - | 32 . . . 253 | 149/223 (66%) | 5e−88 |
| | pig, 200 aa. | 1 . . . 200 | 172/223 (76%) | |
| JE0173 | ribonuclease T2 (EC 3.1.27.1) - | 33 . . . 250 | 126/219 (57%) | 2e−72 |
| | bovine, 198 aa. | 2 . . . 196 | 155/219 (70%) | |

PFam analysis predicts that the NOV55a protein contains the domain shown in the Table 55F.

TABLE 55F
Domain Analysis of NOV55a

| | | Identities/ | |
|---|---|---|---|
| | NOV55a | Similarities | |
| | Match | for the | |
| Pfam Domain | Region | Matched Region | Expect Value |
| ribonuclease_T2 | 39 . . . 219 | 63/212 (30%) | 9.1e−64 |
| | | 149/212 (70%) | |

### EXAMPLE 56

The NOV56 clone was analyzed, and the nucleotide and encoded polypeptide sequences are shown in Table 56A.

TABLE 56A
NOV56 Sequence Analysis

| | |
|---|---|
| | SEQ ID NO: 427 2684 bp |
| NOV56a, | ATCAGAATTTTGAGTTCTAGTATTTACTCTCTCGATTCCTTGTTAATTTAAATGGTAC |
| CG92142-01 | |
| DNA Sequence | CTATTTTTTATAGCACATGATTTGGGAATTACACTTTGTGACATGGATGAATCTGCAC |
| | TGACCCTTGGTACAATAGATGTTTCTTATCTGCCACATTCATCAGAATACAGTGTTGG |
| | TCGATGTAAGCACACAAGTGAGGAATGGGTAGAGTGTGGCTTTAGACCCACCATCTTC |
| | AGATCTGCAACTTTAAAATGGAAAGAAAGCCTAATGAGTCGGAAAAGGCCATTTGTTG |
| | GAAGATGTTGTTACTCCTGCACTCCCCAGAGCTGGGACAAATTTTTCAACCCCAGTAT |
| | CCCGTCTTTGGGTTTGCGGAATGTTATTTATATCAATGAAACTCACACAAGACACCGC |
| | GGATCGCTTGCAAGACGCCTTTCTTACGTTCTTTTTATTCAAGAGCGAGATGTGCATA |
| | AGGGCATGTTTGCCACCAATGTGACTGAAAATGTGCTGAACAGCAGTAGAGTACAAGA |
| | GGCAATTGCAGAAGTGGCTGCTGAATTAAACCCTGATGGTTCTGCCCAGCAGCAATCA |
| | AAAGCCGTTAACAAAGTGAAAAAGAAAGCTAAAAGGATTCTTCAAGAAATGGTTGCCA |
| | CTGTCTCACCGCCAATGATCAGACTGACTGGGTGGGTGCTGCTAAAACTGTTCAACAG |
| | CTTCTTTTGGAACATTCAAATTCACAAAGGTCAACTTGAGATGGTTAAAGCTGCAACT |
| | GAGACGAATTTGCCGCTTCTGTTTCTACCAGTTCATAGATCCCATATTGACTATCTGC |
| | TGCTCACTTTCATTCTCTTCTGCCATAACATCAAAGCACCATACATTGCTTCAGGCAA |
| | TAATCTCAACATCCCAATCTTCAGTACCTTGATCCATAAGCTTGGGGGCTTCTTCATA |
| | CGACGAAGGCTCGATGAAACACCAGATGGACGGAAAGATGTTCTCTATAGAGCTTTGC |
| | TCCATGGGCATATAGTTGAATTACTTCGACAGCAGCAATTCTTGGAGATCTTCCTGGA |
| | AGGCACACGTTCTAGGAGTGGAAAAACCTCTTGTGCTCGGGCAGGACTTTTGTCAGTT |
| | GTGGTAGATACTCTGTCTACCAATGTCATCCCAGACATCTTGATAATACCTGTTGGAA |
| | TCTCCTATGATCGCATTATCGAAGGTCACTACAATGGTGAACAACTGGGCAAACCTAA |
| | GAAGAATGAGAGCCTGTGGAGTGTAGCAAGAGGTGTTATTAGAATGTTACGAAAAAAC |
| | TATGGTTGTGTCCGAGTGGATTTTGCACAGCCATTTTCCTTAAAGGAATATTTAGAAA |
| | GCCAAAGTCAGAAACCGGTGTCTGCTCTACTTTCCCTCGAGCAAGCGTTGTTACCAGC |
| | TATACTTCCTTCAAGACCCAGTGATGCTGCTGATGAAGGTAGAGACACGTCCATTAAT |
| | GAGTCCAGAAATGCAACAGATGAATCCCTACGAAGGAGGTTGATTGCAAATCTGGCTG |
| | AGCATATTCTATTCACTGCTAGCAAGTCCTGTGCCATTATGTCCACACACATTGTGGC |
| | TTGCCTGCTCCTCTACAGACACAGGCAGGGAATTGATCTCTCCACATTGGTCGAAGAC |
| | TTCTTTGTGATGAAAGAGGAAGTCCTGGCTCGTGATTTTGACCTGGGGTTCTCAGGAA |
| | ATTCAGAAGATGTAGTAATGCATGCCATACAGCTGCTGGGAAATTGTGTCACAATCAC |
| | CCACACTAGCAGGAACGATGAGTTTTTTATCACCCCCAGCACAACTGTCCCATCAGTC |
| | TTCGAACTCAACTTCTACAGCAATGGGGTACTTCATGTCTTTATCATGGAGGCCATCA |
| | TAGCTTGCAGCCTTTATGCAGTTCTGAACAAGAGGGGACTGGGGGGTCCCACTAGCAC |
| | CCCACCTAACCTGATCAGCCAGGAGCAGCTGGTGCGGAAGGCGGCCAGCCTGTGCTAC |
| | CTTCTCTCCAATGAAGGCACCATCTCACTGCCTTGCCAGACATTTTACCAAGTCTGCC |
| | ATGAAACAGTAGGAAAGTTTATCCAGTATGGCATTCTTACAGTGGCAGAGCACGATGA |
| | CCAGGAAGATATCAGTCCTAGTCTTGCTGAGCAGCAGTGGGACAAGAAGCTTCCTGAA |
| | CCTTTGTCTTGGAGAAGTGATGAAGAAGATGAAGACAGTGACTTTGGGGAGGAACAGC |
| | GAGATTGCTACCTGAAGGTGAGCCAATCCAAGGAGCACCAGCAGTTTATCACCTTCTT |
| | ACAGAGACTCCTTGGGCCTTTGCTGGAGGCCTACAGCTCTGCTGCCATCTTTGTTCAC |
| | AACTTCAGTGGTCCTGTTCCAGAACCTGAGTATCTGCAAAAGTTGCACAAATACCTAA |
| | TAACCAGAACAGAAAGAAATGTTGCAGTATATGCTGAGAGTGCCACATATTGTCTTGT |
| | GAAGAATGCTGTGAAAATGTTTAAGGATATTGGGGTTTTCAAGGAGACCAAACAAAAG |
| | AGAGTGTCTGTTTTAGAACTGAGCAGCACTTTTCTACCTCAATGCAACCGACAAAAAC |
| | TTCTAGAATATATTCTGAGTTTTGTGGTGCTGTAGGTAACGTGTGGCACTGCTCGCAA |
| | ATGAAGGTCATGAGATGAGTTCCTTGTAGGTACCAGCTTCTGGCTCAAGAGTTGAAGG |
| | TGCCGTCGCAGGGTCA |
| | ORF Start: ATG at 101 ORF Stop: TAG at 2585 |
| | SEQ ID NO: 428 828 aa MW at 93835.7kD |
| NOV56a, | MDESALTLGTIDVSYLPHSSEYSVGRCKHTSEEWVECGFRPTIFRSATLKWKESLMSR |
| CG92142-01 | |
| Protein Sequence | KRPFVGRCCYSCTPQSWDKFFNPSIPSLGLRNVIYINETHTRHRGWLARRLSYVLFIQ |
| | ERDVHKGMFATNVTENVLNSSRVQEAIAEVAAELNPDGSAQQQSKAVNKVKKKAKRIL |
| | QEMVATVSPAMIRLTGWVLLKLFNSFFWNIQIHKGQLEMVKAATETNLPLLFLPVHRS |
| | HIDYLLLTFILFCHNIKAPYIASGNNLNIPIFSTLIHKLGGFFIRRRLDETPDGRKDV |
| | LYRALLHGHIVELLRQQQFLEIFLEGTRSRSGKTSCARAGLLSVVVDTLSTNVIPDIL |
| | IIPVGISYDRIIEGHYNGEQLGKPKKNESLWSVARGVIRMLRKNYGCVRVDFAQPFSL |
| | KEYLESQSQKPVSALLSLEQALLPAILPSRPSDAADEGRDTSINESRNATDESLRRRL |
| | IANLAEHILFTASKSCAIMSTHIVACLLLYRHRQGIDLSTLVEDFFVMKEEVLARDFD |
| | LGFSCNSEDVVMHAIQLLGNCVTITHTSRNDEFFITPSTTVPSVFELNFYSNGVLHVF |
| | IMEAIIACSLYAVLNKRGLGGPTSTPPNLISQEQLVRKAASLCYLLSNEGTISLPCQT |
| | FYQVCHETVGKFIQYGILTVAEHDDQEDISPSLAEQQWDKKLPEPLSWRSDEEDEDSD |
| | FGEEQRDCYLKVSQSKEHQQFITFLQRLLGPLLEAYSSAAIFVHNFSGPVPEPEYLQK |
| | LHKYLITRTERNVAVYAESATYCLVKNAVKMFKDIGVFKETKQKRVSVLELSSTFLPQ |
| | CNRQKLLEYILSFVVL |
| | SEQ ID NO: 429 2527 bp |
| NOV56b, | GCACATGATTTGGGAATTACACTTTGTGACATGGATGAATCTGCACTGACCCTTGGTA |
| CG92142-02 | |
| DNA Sequence | CAATAGATGTTTCTTATCTGCCACATTCATCAGAATACAGTGTTGGTCGATGTAAGCA |
| | CACAAGTGAGGAATGGGGTGAGTGTGGCTTTAGACCCACCGTCTTCAGATCTGCAACT |
| | TTAAAATGGAAAGAAAGCCTAATGAGTCGGAAAAGGCCATTTGTTGGAAGATGTTGTT |
| | ACTCCTGCACTCCCCAGAGCTGGGACAAATTTTTCAACCCCAGTATCCCGTCTTTGGG |
| | TTTGCGGAATGTTATTTATATCAATGAAACTCACACAAGACACCGCGGATGGCTTGCA |
| | AGACGCCTTTCTTACGTTCTTTTTATTCAAGAGCGAGATGTGCATAAGGGCATGTTTG |
| | CCACCAATGTGACTGGAAATGTGCTGAACAGCAGTAGAGTACAAGAGGCAATTGCAGA |
| | AGTGGCTGCTGAATTAAACCCTGATGGTTCTGCCCAGCAGCAATCAAAAGCCGTTAAC |
| | AAAGTGAAAAAGAAAGCTAAAAGGATTCTTCAAGAAATGGTTGCCACTGTCTCACCGG |
| | CAATGATCAGACTGACTGGGTGGGTGCTGCTAAAACTGTTCAACAGCTTCTTTTCGAA |
| | CATTCAAATTCACAAAGGTCAACTTGAGATGGTTAAAGCTGCAACTGAGACGAATTTG |
| | CCGCTTCTGTTTCTACCAGTTCATAGATCCCATATTGACTATCTGCTGCTCACTTTCA |
| | TTCTCTTCTGCCATAACATCAAAGCACCATACATTGCTTCAGGCAATAATCTCAACAT |
| | CCCAATCTTCAGTACCTTGATCCATAAGCTTGGGGGCTTCTTCATACGACGAAGGCTC |
| | GATGAAACACCAGATGGACGGAAAGATGTTCTCTATAGAGCTTTGCTCCATGGGCATA |
| | TAGTTGAATTACTTCGACAGCAGCAATTCTTCGAGATCTTCCTGGAAGGCACACGTTC |
| | TAGGAGTGGAAAAACCTCTTGTGCTCGGGCAGGACTTTTGTCAGTTGTGGTAGATACT |
| | CTGTCTACCAATGTCATCCCAGACATCTTGATAATACCTGTTGGAATCTCCTATGATC |
| | GCATTATCGAAGGTCACTACAATGGTGAACAACTGGGCAAACCTAAGAAGAATGAGAG |
| | CCTGTGGAGTGTAGCAAGAGGTGTTATTAGAATGTTACGAAAAAACTATGGTTGTGTC |
| | CGAGTGGATTTTGCACAGCCATTTTCCTTAAAGGAATATTTAGAAAGCCAAAGTCAGA |
| | AACCGGTGTCTGCTCTACTTTCCCTGGAGCAAGCGTTGTTACCAGCTATACTTCCTTC |
| | AAGACCCAGTGATGCTGCTGATGAAGGTAGAGACACGTCCATTAATGAGTCCAGAAAT |
| | GCAACAGATGAATCCCTACGAAGGAGGTTGATTGCAAATCTGGCTGAGCATATTCTAT |
| | TCACTGCTAGCAAGTCCTGTGCCATTATGTCCACACACATTGTGGCTTGCCTGCTCCT |
| | CTACAGACACAGGCAGGGAATTGATCTCTCCACATTGGTCGAAGACTTCTTTGTGATG |
| | AAAGAGGAAGTCCTGGCTCGTGATTTTGACCTGGGGTTCTCAGGAAATTCAGAAGATG |
| | TAGTAATGCATGCCATACAGCTGCTGGGAAATTGTGTCACAATCACCCACACTAGCAG |
| | GAATGATGAGTTTTTTATCACCCCCAGCACAACTGTCCCATCAGTCTTCGAACTCAAC |
| | TTCTACAGCAATGGGGTACTTCATGTCTTTATCATGGAGGCCATCATAGCTTGCAGCC |
| | TTTATGCAGTTCTGAACAAGAGGGGACTGGGGGGTCCCACTAGCACCCCACCTAACCT |
| | GATCAGCCAGGAGCAGCTGGTGCGGAAGGCGGCCAGCCTGTGCTACCTTCTCTCCAAT |
| | GAAGGCACCATCTCACTGCCTTGCCAGACATTTTACCAAGTCTGCCATGAAACAGTAG |
| | GAAAGTTTATCCAGTATGGCATTCTTACAGTGGCAGAGCACGATGACCAGGAAGATAT |
| | CAGTCCTAGTCTTGCTGAGCAGCAGTGGGACAAGAAGCTTCCTGAACCTTTGTCTTGG |
| | AGAAGTGATGAAGAAGATGAAGACAGTGACTTTGGGGAGGAACAGCGAGATTGCTACC |
| | TGAAGGTGAGCCAATCCAACGAGCACCAGCAGTTTATCACCTTCTTACAGAGACTCCT |
| | TGGGCCTTTGCTGGAGGCCTACAGCTCTGCTGCCATCTTTGTTCACAACTTCAGTGGT |
| | CCTGTTCCAGAACCTGAGTATCTGCAAAAGTTGCACAAATACCTAATAACCAGAACAG |
| | AAAGAAATGTTGCAGTATATGCTGAGAGTGCCACATATTGTCTTGTGAAGAATGCTGT |
| | GAAAATGTTTAAGGATATTGGGGTTTTCAAGGAGACCAAACAAAAGAGAGTGTCTGTT |
| | TTAGAACTGAGCAGCACTTTTCTACCTCAATGCAACCGACAAAGACTTCTAGAATATA |
| | TTCTGAGTTTTGTGGTGCTGTAAGTAACGTCTG |
| | ORF Start: ATG at 31 ORF Stop: TAA at 2515 |
| | SEQ ID NO: 430 828 aa Mw at 93735.6kD |
| NOV56b, | MDESALTLGTTDVSYLPHSSEYSVCRCKHTSEEWGECGFRPTVFRSATLKWKESLMSR |
| CG92142-02 | |
| Protein Sequence | KRPFVGRCCYSCTPQSWDKFFNPSIPSLGLRNVIYINETHTRHRGWLARRLSYVLFIQ |
| | ERDVHKGMFATNVTGNVLNSSRVQEAIAEVAAELNPDGSAQQQSKAVNKVKKKAKRIL |
| | QEMVATVSPAMIRLTGWVLLKLFNSFFWNIQIHKGQLEMVKAATETNLPLLFLPVHRS |
| | HIDYLLLTFILFCHNIKAPYIASGNNLNIPIFSTLIHKLGGFFIRRRLDETPDGRKDV |
| | LYRALLHGHIVELLRQQQFLEIFLEGTRSRSGKTSCARAGLLSVVVDTLSTNVIPDIL |
| | IIPVGISYDRIIEGHYNGEQLGKPKKNESLWSVARGVIRMLRKNYGCVRVDFAQPFSL |
| | KEYLESQSQKPVSALLSLEQALLPAILPSRPSDAADEGRDTSINESRNATDESLRRRL |
| | IANLAEHILFTASKSCAIMSTHIVACLLLYRHRQGIDLSTLVEDFFVMKEEVLARDFD |
| | LGFSGNSEDVVMHAIQLLGNCVTITHTSRNDEFFITPSTTVPSVFELNFYSNGVLHVF |
| | IMEAIIACSLYAVLNKRGLGGPTSTPPNLISQEQLVRKAASLCYLLSNEGTISLPCQT |
| | FYQVCHETVGKFIQYGILTVAEHDDQEDISPSLAEQQWDKKLPEPLSWRSDEEDEDSD |
| | FGEEQRDCYLKVSQSKEHQQFITFLQRLLGPLLEAYSSAAIFVHNFSGPVPEPEYLQK |
| | LHKYLITRTERNVAVYAESATYCLVKNAVKMFKDIGVFKETKQKRVSVLELSSTFLPQ |
| | CNRQRLLEYILSFVVL |

Sequence comparison of the above protein sequences yields the following sequence relationships shown in Table 56B.

TABLE 56B
Comparison of NOV56a against NOV56b.

| | | Identities/ |
|---|---|---|
| | NOV56a Residues/ | Similarities |
| | Match | for the |
| Protein Sequence | Residues | Matched Region |
| NOV56b | 1 . . . 828 | 824/828 (99%) |
| | 1 . . . 828 | 826/828 (99%) |

Further analysis of the NOV56a protein yielded the following properties shown in Table 56C.

TABLE 56C
Protein Sequence Properties NOV56a

| | |
|---|---|
| PSort | 0.8500 probability located in endoplasmic reticulum |
| analysis: | (membrane); 0.4400 probability located in plasma |
| | membrane; 0.3000 probability located in nucleus; |
| | 0.1000 probability located in mitochondrial inner |
| | membrane |
| SignalP | No Known Signal Sequence Predicted |
| analysis: | |

A search of the NOV56a protein against the Geneseq database, a proprietary database that contains sequences published in patents and patent publication, yielded several homologous proteins shown in Table 56D.

TABLE 56D
Geneseq Results for NOV56a

| | | NOV56a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| ABG66665 | Human glycerol-3-phosphate | 1 . . . 828 | 827/828 (99%) | 0.0 |
| | acyltransferase hGPAT - Homo | 1 . . . 828 | 827/828 (99%) | |
| | sapiens, 828 aa. [WO200240666- | | | |
| | A2, 23 MAY 2002] | | | |
| AAE22144 | Human TRNFR-6 protein - Homo | 1 . . . 828 | 827/828 (99%) | 0.0 |
| | sapiens, 828 aa. [WO200226950- | 1 . . . 828 | 827/828 (99%) | |
| | A2, 04 APR. 2002] | | | |
| AAU78393 | Human acyltransferase, ACTR-1 - | 1 . . . 828 | 826/828 (99%) | 0.0 |
| | Homo sapiens, 828 aa. | 1 . . . 828 | 827/828 (99%) | |
| | [WO200216592-A2, 28 FEB. 2002] | | | |
| AAE22145 | Human TRNFR-7 protein - Homo | 56 . . . 826 | 262/790 (33%) | e−102 |
| | sapiens, 801 aa. [WO200226950- | 40 . . . 799 | 403/790 (50%) | |
| | A2, 04 APR. 2002] | | | |
| ABB61594 | Drosophila melanogaster | 163 . . . 809 | 196/654 (29%) | 4e−82 |
| | polypeptide SEQ ID NO 11574 - | 194 . . . 820 | 353/654 (53%) | |
| | Drosophila melanogaster, 850 aa. | | | |
| | [WO200171042-A2, 27 SEP. 2001] | | | |

In a BLAST search of public sequence datbases, the NOV56a protein was found to have homology to the proteins shown in the BLASTP data in Table 56E.

TABLE 56E
Public BLASTP Results for NOV56a

| | | NOV56a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9HCL2 | Glycerol-3-phosphate | 1 . . . 828 | 828/828 (100%) | 0.0 |
| | acyltransferase, mitochondrial | 1 . . . 828 | 828/828 (100%) | |
| | precursor (EC 2.3.1.15) (GPAT) - | | | |
| | Homo sapiens (Human), 828 aa. | | | |
| AAH30783 | KIAA1560 protein - Homo sapiens | 1 . . . 828 | 825/828 (99%) | 0.0 |
| | (Human), 828 aa. | 1 . . . 828 | 825/828 (99%) | |
| Q8VCT2 | Glycerol-3-phosphate | 1 . . . 828 | 769/828 (92%) | 0.0 |
| | acyltransferase, mitochondrial - | 1 . . . 827 | 799/828 (95%) | |
| | Mus musculus (Mouse), 827 aa. | | | |
| Q61586 | Glycerol-3-phosphate | 1 . . . 828 | 767/828 (92%) | 0.0 |
| | acyltransferase, mitochondrial | 1 . . . 827 | 799/828 (95%) | |
| | precursor (EC 2.3.1.15) (GPAT) | | | |
| | (P90) - Mus musculus (Mouse), | | | |
| | 827 aa. | | | |
| P97564 | Glycerol-3-phosphate | 1 . . . 828 | 760/828 (91%) | 0.0 |
| | acyltransferase, mitochondrial | 1 . . . 828 | 794/828 (95%) | |
| | precursor (EC 2.3.1.15) (GPAT) - | | | |
| | Rattus norvegicus (Rat), 828 aa. | | | |

PFam analysis predicts that the NOV56a protein contains the domains shown in the Table 56F.

TABLE 56F
Domain Analysis of NOV56a

| | | Identities/ | |
|---|---|---|---|
| | NOV56a | Similarities | |
| | Match | for the | |
| Pfam Domain | Region | Matched Region | Expect Value |
| Acyltransferase | 215 . . . 412 | 47/207 (23%) | 6.4e−34 |
| | | 151/207 (73%) | |

### EXAMPLE 57

The NOV57 clone was analyzed, and the nucleotide and encoded polypeptide sequences 5 are shown in Table 57A.

TABLE 57A
NOV57 Sequence Analysis

| | |
|---|---|
| | SEQ ID NO: 431 1538 bp |
| NOV57a, | CACCGAGCCTCACGGGAGCTGATGGCTGCAAAGAAGACCCACACCTCACAAATTGAAG |
| CG95765-01 | |
| DNA Sequence | TGATCCCTTGCAAAATCTGTGGGGACAAGTCGTCTGGGATCCACTACGGGGTTATCAC |
| | CTGTGAGGGGTGCAAGGGCTTCTTCCGGCCTACTCCTGCACCCGTCAGCAGAACTGCC |
| | CCATCGACCGCACCAGCCGAAACCGATGCCAGCACTGCCGCCTGCAGAAATGCCTGGC |
| | GCTGGGGATGTCCCGAGATGCTGTCAAGTTCGGCCGCATGTCCAAGAAGCAGAGGGAC |
| | AGCCTGCATGCAGAAGTGCAGAAACAGCTGCAGCAGCGGCAACAGCAGCAACAGGAAC |
| | CAGTGGTCAAGACCCCTCCAGCAGGGGCCCAAGGAGCAGATACCCTCACCTACACCTT |
| | GGGGCTCCCAGACGGGCAGCTGCCCCTGGGCTCCTCGCCTGACCTGCCTGAGGCTTCT |
| | GCCTGTCCCCCTGGCCTCCTGAAAGCCTCAGGCTCTGGGCCCTCATATTCCAACAACT |
| | TGGCCAAGGCAGGGCTCAATGGGGCCTCATGCCACCTTGAATACAGCCCTGAGCGGGG |
| | CAAGGCTGAGGGCAGAGAGAGCTTCTATAGCACAGGCAGCCAGCTGACCCCTGACCGA |
| | TGTGGACTTCGTTTTGAGGAACACAGGCATCCTGGGCTTGGGGAACTGGGACAGGGCC |
| | CAGACAGCTACGGCAGCCCCAGTTTCCGCAGCACACCGGAGGCACCCTATGCCTCCCT |
| | GACAGAGATAGAGCACCTGGTGCAGAGCGTCTGCAAGTCCTACAGGGAGACATGCCAG |
| | CTGCGGCTGGAGGACCTGCTGCGGCAGCGCTCCAACATCTTCTCCCGGGAGGAAGTGA |
| | CTGGCTACCAGAGGAAGTCCATGTGGGAGATGTGGGAACGGTGTGCCCACCACCTCAC |
| | CGAGGCCATTCAGTACGTGGTGGAGTTCGCCAAGAGGCTCTCAGGCTTTATGGAGCTC |
| | TGCCAGAATGACCAGATTGTGCTTCTCAAAGCAGGAGCAATGGAAGTGGTGCTGGTTA |
| | GGATGTGCCGGGCCTACAATGCCAACAACCACACAGTCTTTTTTGAAGGCAAATACGG |
| | TGGTGTGGAGCTGTTTCGAGCCTTGGGCTGCAGCGAGCTCATCAGCTCCATATTTGAC |
| | TTTTCCCACTTCCTCAGCGCCCTGTGTTTTTCCGAGGATGAGATTGCCCTCTACACGG |
| | CCCTTGTTCTCATCAATGCCAACCGTCCTGGGCTCCAAGAGAAGAGGAGAGTGGAACA |
| | TCTGCAATACAATTTGGAACTGGCTTTCCATCATCATCTCTGCAAGACTCATCGACAA |
| | AGCATCCTGGCAAAGCTGCCACCCAAAGGAAAACTCCGGAGCCTGTGTAGCCAGCATG |
| | TGGAAAGGCTGCAGATCTTCCAGCACCTCCACCCCATCGTGGTCCAAGCCGCTTTCCC |
| | TCCACTCTACAAGGAGCTCTTCAGCACTGAAACCGAGTCACCTGTGGGGCTGTCCAAG |
| | TGACCTGGAAGAGGGACTCCTTGCCTCTCC |
| | ORF Start: ATG at 240 ORF Stop: TGA at 1509 |
| | SEQ ID NO: 432 423 aa MW at 47418.4kD |
| NOV57a, | MSRDAVKFGRMSKKQRDSLHAEVQKQLQQRQQQQQEPVVKTPPAGAQGADTLTYTLGL |
| CG95765-01 | |
| Protein Sequence | PDGQLPLGSSPDLPEASACPPGLLKASGSGPSYSNNLAKAGLNGASCHLEYSPERGKA |
| | EGRESFYSTGSQLTPDRCGLRFEEHRHPGLGELGQGPDSYGSPSFRSTPEAPYASLTE |
| | IEHLVQSVCKSYRETCQLRLEDLLRQRSNIFSREEVTGYQRKSMWEMWERCAHHLTEA |
| | IQYVVEFAKRLSGFMELCQNDQTVLLKAGAMEVVLVRMCRAYNANNHTVFFEGKYGGV |
| | ELFRALGCSELISSIFDFSHFLSALCFSEDEIALYTALVLINANRPGLQEKRRVEHLQ |
| | YNLELAFHHHLCKTHRQSILAKLPPKGKLRSLCSQHVERLQIFQHLNPIVVQAAFPPL |
| | YKELFSTETESPVGLSK |
| | SEQ ID NO: 433 1819 bp |
| NOV57b, | CCCCTGGGCCCTGCTCCCTGCCCTCCTGGGCAGCCAGGGCAGCCAGGACGGCACCAAG |
| CG95765-02 | |
| DNA Sequence | GGAGCTGCCCCATGGACAGGGCCCCACAGAGACAGCACCGAGCCTCACGGGAGCTGCT |
| | GGCTGCAAAGAAGACCCACACCTCACAAATTGAAGTGATCCCTTGCAAAATCTGTGGG |
| | GACAAGTCGTCTGGGATCCACTACGGGGTTATCACCTGTGAGGGGTGCAAGGGCTTCT |
| | TCCGCCGGAGCCAGCGCTGTAACGCGGCCTACTCCTGCACCCGTCAGCAGAACTGCCC |
| | CATCGACCGCACCAGCCGAAACCGATGCCAGCACTGCCGCCTGCAGAAATGCCTGGCG |
| | CTGGGGATGTCCCGAGATGCTGTCAAGTTCGGCCGCATGTCCAAGAAGCAGAGGGACA |
| | GCCTGCATGCAGAAGTGCAGAAACAGCTGCAGCAGCGGCAACAGCAGCAACAGGAACC |
| | AGTGGTCAAGACCCCTCCAGCAGGGGCCCAAGGAGCAGATACCCTCACCTACACCTTG |
| | GGGCTCCCAGACGGGCAGCTCCCCCTGGGCTCCTCGCCTCACCTGCCTGAGGCTTCTG |
| | CCTGTCCCCCTGGCCTCCTGAAAGCCTCAGCCTCTGGGCCCTCATATTCCAACAACTT |
| | GGCCAAGGCAGGGCTCAATGGGGCCTCATGCCACCTTGAATACAGCCCTGAGCGGGGC |
| | AAGGCTGAGGGCAGAGAGAGCTTCTATAGCACAGGCAGCCAGCTGACCCCTGACCGAT |
| | GTGGACTTCGTTTTGAGGAACACAGGCATCCTGGGCTTGGGGAACTGGGACAGGGCCC |
| | AGACAGCTACGGCAGCCCCAGTTTCCGCAGCACACCGGAGGCACCCTATGCCTCCCTG |
| | ACAGAGATAGAGCACCTGGTGCAGAGCGTCTGCAAGTCCTACAGGGAGACATGCCAGC |
| | TGCGGCTGGAGGACCTGCTGCGGCAGCGCTCCAACATCTTCTCCCGGGAGGAAGTGAC |
| | TGGCTACCAGAGGAAGTCCATGTGGGAGATGTGGGAACGGTGTGCCCACCACCTCACC |
| | GAGGCCATTCAGTACGTGGTGGAGTTCGCCAAGAGGCTCTCACGCTTTATGGAGCTCT |
| | GCCAGAATGACCAGATTGTGCTTCTCAAAGCAGGAGCAATGGAAGTGGTGCTGGTTAG |
| | GATGTGCCGGGCCTACAATGCTGACAACCGCACGGTCTTTTTTGAAGGCAAATACGGT |
| | GGCATGGAGCTGTTCCGAGCCTTGGGCTGCAGCGAGCTCATCAGCTCCATCTTTGACT |
| | TCTCCCACTCCCTAAGTGCCTTGCACTTTTCCGAGGATGAGATTGCCCTCTACACAGC |
| | CCTTGTTCTCATCAATGCCCATCGGCCAGGGCTCCAAGAGAAAAGGAAACTAGAACAG |
| | CTGCAGTACAATCTGGAGCTGGCCTTTCATCATCATCTCTGCAAGACTCATCGCCAAA |
| | GCATCCTGGCAAACCTGCCACCCAAGGGGAAGCTTCGGAGCCTGTGTAGCCAGCATGT |
| | GGAAAGGCTGCAGATCTTCCAGCACCTCCACCCCATCGTGGTCCAAGCCGCTTTCCCT |
| | CCACTCTACAAGGAGCTCTTCAGCACTGAAACCGAGTCACCTGTGGGCTGTCCAAGTG |
| | ACCTGGAAGAGGGACTCCTTGCCTCTCCCTATGGCCTGCTGGCCACCTCCCTGGACCC |
| | CGTTCCACCCTCACCCTTTTCCTTTCCCATGAACCCTGGAGGGTGGTCCCCACCAGCT |
| | CTTTGGAAGTGAGCAGATGCTGCGGCTGGCTTTCTGTCAGCAGGCCGGCCTGGCAGTG |
| | GGACAATCGCCAGAGGGTGGG |
| | ORF Start: ATG at 70 ORF Stop: TGA at 1750 |
| | SEQ ID NO: 434 560 aa MW at 62588.6kD |
| NOV57b, | MDRAPQRQHRASRELLAAKKTHTSQIEVIPCKICGDKSSGIHYGVITCEGCKGFFRRS |
| CG95765-02 | |
| Protein Sequence | QRCNAAYSCTRQQNCPIDRTSRNRCQHCRLQKCLALGMSRDAVKFGRMSKKQRDSLHA |
| | EVQKQLQQRQQQQQEPVVKTPPAGAQGADTLTYTLGLPDGQLPLGSSPDLPEASACPP |
| | GLLKASGSGPSYSNNLAKAGLNGASCHLEYSPERGKAEGRESFYSTGSQLTPDRCGLR |
| | FEEHRHPGLGELGQGPDSYGSPSFRSTPEAPYASLTEIEHLVQSVCKSYRETCQLRLE |
| | DLLRQRSNIFSREEVTGYQRKSMWEMWERCAHLLTEAIQYVVEFAKRLSGFMELCQND |
| | QIVLLKAGANEVVLVRMCRAYNADNRTVFFEGKYGGMELFRALGCSELISSIFDFSHS |
| | LSALHFSEDEIALYTALVLINAHRPGLQEKRKVEQLQYNLELAFHHHLCKTHRQSILA |
| | KLPPKGKLRSLCSQHVERLQIFQHLHPIVVQAAFPPLYKELFSTETESPVGCPSDLEE |
| | GLLASPYGLLATSLDPVPPSPFSFPMNPGGWSPPALWK |

Sequence comparison of the above protein sequences yields the following sequence relationships shown in Table 57B.

TABLE 57B
Comparison of NOV57a against NOV57b.

| | NOV57a | Identities/ |
|---|---|---|
| | Residues/ | Similarities |
| | Match | for the |
| Protein Sequence | Residues | Matched Region |
| NOV57b | 1 . . . 420 | 412/420 (98%) |
| | 96 . . . 515 | 416/420 (98%) |

Further analysis of the NOV57a protein yielded the following properties shown in Table 57C.

TABLE 57C
Protein Sequence Properties NOV57a

| | |
|---|---|
| PSort | 0.3600 probability located in mitochondrial matrix |
| analysis: | space; 0.3000 probability located in microbody |
| | (peroxisome); 0.1000 probability located in lysosome |
| | (lumen); 0.0000 probability located in endoplasmic |
| | reticulum (membrane) |
| SignalP | No Known Signal Sequence Predicted |
| analysis: | |

A search of the NOV57a protein against the Geneseq database, a proprietary database that contains sequences published in patents and patent publication, yielded several homologous proteins shown in Table 57D.

TABLE 57D
Geneseq Results for NOV57a

| | | NOV57a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB03062 | Human retinoid-like orphan | 1 . . . 423 | 415/423 (98%) | 0.0 |
| | receptor-gamma 60 kD isoform - | 96 . . . 518 | 419/423 (98%) | |
| | Homo sapiens, 518 aa. | | | |
| | [WO200024757-A1, 04 MAY | | | |
| | 2000] | | | |
| AAB03066 | Human ROR-gamma 60 kD isoform | 1 . . . 423 | 414/423 (97%) | 0.0 |
| | polymorphic variant #1, L516I - | 96 . . . 518 | 419/423 (98%) | |
| | Homo sapiens, 518 aa. | | | |
| | [WO200024757-A1, 04 MAY | | | |
| | 2000] | | | |
| AAB03069 | Human ROR-gamma 60 kD isoform | 1 . . . 423 | 414/423 (97%) | 0.0 |
| | polymorphic variant #3, K518R - | 96 . . . 518 | 419/423 (98%) | |
| | Homo sapiens, 518 aa. | | | |
| | [WO200024757-A1, 04 MAY | | | |
| | 2000] | | | |
| AAB03068 | Human ROR-gamma 60 kD isoform | 1 . . . 423 | 414/423 (97%) | 0.0 |
| | polymorphic variant #2 - Homo | 96 . . . 518 | 419/423 (98%) | |
| | sapiens, 518 aa. [WO200024757- | | | |
| | A1, 04 MAY 2000] | | | |
| AAB03067 | Human ROR-gamma 60 kD isoform | 1 . . . 423 | 414/423 (97%) | 0.0 |
| | polymorphic variant #1, L516V - | 96 . . . 518 | 419/423 (98%) | |
| | Homo sapiens, 518 aa. | | | |
| | [WO200024757-A1, 04 MAY | | | |
| | 2000] | | | |

In a BLAST search of public sequence datbases, the NOV57a protein was found to have homology to the proteins shown in the BLASTP data in Table 57E.

TABLE 57E
Public BLASTP Results for NOV57a

| | | | Identities/ | |
|---|---|---|---|---|
| Protein | | | Similarities for | |
| Accession | | NOV57a Residues/ | the Matched | Expect |
| Number | Protein/Organism/Length | Match Residues | Portion | Value |
| CAD38900 | Hypothetical protein - Homo | 1 . . . 423 | 415/423 (98%) | 0.0 |
| | sapiens (Human), 497 aa. | 75 . . . 497 | 419/423 (98%) | |
| AAH31554 | Hypothetical protein - Homo | 1 . . . 423 | 415/423 (98%) | 0.0 |
| | sapiens (Human), 518 aa. | 96 . . . 518 | 419/423 (98%) | |
| P51449 | Nuclear receptor ROR-gamma | 1 . . . 420 | 412/420 (98%) | 0.0 |
| | (Nuclear receptor RZR-gamma) - | 96 . . . 515 | 416/420 (98%) | |
| | Homo sapiens (Human), 560 aa. | | | |
| Q91YT5 | RAR-related orphan receptor | 1 . . . 423 | 378/423 (89%) | 0.0 |
| | gamma - Mus musculus (Mouse), | 96 . . . 516 | 395/423 (93%) | |
| | 516 aa. | | | |
| Q9R177 | RORgamma t - Mus musculus | 1 . . . 423 | 378/423 (89%) | 0.0 |
| | (Mouse), 495 aa. | 75 . . . 495 | 395/423 (93%) | |

PFam analysis predicts that the NOV57a protein contains the domains shown in the Table 57F.

TABLE 57F
Domain Analysis of NOV57a

| | | Identities/ | |
|---|---|---|---|
| | | Similarities for | |
| Pfam | NOV57a | the Matched | Expect |
| Domain | Match Region | Region | Value |
| hormone_rec | 230 . . . 411 | 56/210 (27%) | 1.1e−34 |
| | | 138/210 (66%) | |

### EXAMPLE 58

The NOV58 clone was analyzed, and the nucleotide and encoded polypeptide sequences are shown in Table 58A.

TABLE 58A
NOV58 Sequence Analysis

| | |
|---|---|
| | SEQ ID NO: 435 1712 bp |
| NOV58a, | AAGGTCAATGATAGCATCTGCCTAGAGTCAAACCTCCGTGCTTCTCAGACAGTGCCTT |
| CG97178-01 | |
| DNA Sequence | TTCACCATGAGTGGGTGCCCATTTTTAGGAAACAACTTTGGATATACTTTTAAAAAAC |
| | TCCCCGTAGAAGGCAGCGAAGAAGACAAATCACAAACTGGTGTGAATAGAGCCAGCAA |
| | AGGAGGTCTTATCTATGGCAACTACCTGCATTTGGAAAAAGTTTTGAATGCACAAGAA |
| | CTGCAAAGTGAAACAAAAGGAAATAAAATCCATGATGAACATCTTTTTATCATAACTC |
| | ATCAAGCTTATGAACTCTGGTTTAAGCAAATCCTCTGGGAGTTGGATTCTGTTCGAGA |
| | GATCTTTCAGAATGGCCATGTCAGAGATGAAAGGAACATGCTTAAGGTTGTTTCTCGG |
| | ATGCACCGAGTGTCAGTGATCCTGAAACTGCTGGTGCAGCAGTTTTCCATTCTGGAGA |
| | CGATGACAGCCTTGGACTTCAATGACTTCAGAGAGTACTTATCTCCAGCATCAGGCTT |
| | CCAGAGTTTGCAATTCCGACTATTAGAAAACAAGATAGGTGTTCTTCAGAACATGAGA |
| | GTCCCTTATAACAGAAGACATTATCGTGATAACTTCAAAGGAGAAGAAAATGAACTGC |
| | TACTTAAATCTGAGCAGGAAAAGACACTTCTGGAATTAGTGGAGGCATGGCTGGAAAG |
| | AACTCCAGGTTTAGAGCCACATGGATTTAACTTCTGGGGAAAGCTTGAAAAAAATATC |
| | ACCAGAGGCCTGGAAGAGGAATTCATAAGGATTCAGGCTAAAGAAGAGTCTGAAGAAA |
| | AAGAGGAACAGGTGGCTGAATTTCAGAAGCAAAAAGAGGTGCTACTGTCCTTATTTGA |
| | TGAGAAACGTCATGAACATCTCCTTAGTAAAGGTGAAAGACGGCTGTCATACAGAGCA |
| | CTTCAGGGAGCATTGATGATATATTTTTACAGGGAAGAGCCTAGGTTCCAGGTGCCTT |
| | TTCAGTTGCTGACTTCTCTTATGGACATAGATTCACTGATGACCAAATGGAGATATAA |
| | CCATGTGTGCATGGTGCACAGAATGCTGGGCAGCAAAGCTGGCACCGGTGGTTCCTCA |
| | GGCTATCACTACCTGCGATCAACTGTGAGTGATAGGTACAAGGTATTTGTAGATTTAT |
| | TTAATCTTTCAACATACCTGATTCCCCGACACTGGATACCGAAGATGAACCCAACCAT |
| | TCACAAATTTCTATATACAGCAGAATACTGTGATAGCTCCTACTTCAGCAGTGATGAA |
| | TCAGATTAAAATCGTCTGCAAAATCTATGAAGAATACTGGTTTCACAGCCTATTTTTT |
| | ATTTTCTATGGATTTTCATAAATACAGTTTGAATATATGTATGCATATATTGTTCAGC |
| | ACCACGATGCTCTGATTTAATTCTAGAAACAATTTGATTACCTCTTGTTTGTGACAAG |
| | ACTAAGCATTAAGATGAGAAAGAATACATTTAAATAGTAACATTGTACATAGGGTGTT |
| | TTCCTATTAAAAATCAGTTTCCCCTGAGACTTAATGTAACCACTTAATGTAATCACTA |
| | TCTCATTGTTTCATCTTTATAAACTTGTAAACTTCATCTATTTCAAATATTTTATGCA |
| | GTACATTATATTATTCTGTACAAAGGCTTTCAAACAAAATTTTTAAAATAATAAAGTA |
| | TTAATCTTTCAAAAAAAAAAAAAAAAAAA |
| | ORF Start: ATG at 65 ORF Stop: TAA at 1283 |
| | SEQ ID NO: 436 406 aa MW at 47871.1kD |
| NOV58a, | MSGCPFLGNNFGYTFKKLPVEGSEEDKSQTGVNRASKGGLIYGNYLHLEKVLNAQELQ |
| CG97178-O1 | |
| Protein Sequence | SETKGNKIHDEHLFTITHQAYELWFKQILWELDSVREIFQNGHVRDERNMLKVVSRMH |
| | RVSVILKLLVQQFSILETMTALDFNDFREYLSPASGFQSLQFRLLENKIGVLQNMRVP |
| | YNRRHYRDNFKGEENELLLKSEQEKTLLELVEAWLERTPGLEPHGFNFWGKLEKNITR |
| | GLEEEFIRIQAKEESEEKEEQVAEFQKQKEVLLSLFDEKRHEHLLSKGERRLSYRALQ |
| | GALMIYFYREEPRFQVPFQLLTSLMDIDSLMTKWRYNHVCMVHRMLGSKAGTGGSSGY |
| | HYLRSTVSDRYKVFVDLFNLSTYLIPRHWIPKMNPTIHKFLYTAEYCDSSYFSSDESD |
| | SEQ ID NO: 437 1298 bp |
| NOV58b, | CTGCTTCTCAGACAGTGCCTTTTCACCATGAGTGGGTGCCCATTTTTAGGAAACAACT |
| CG97178-02 | |
| DNA Sequence | TTGGATATACTTTTAAAAAACTCCCCGTAGAAGGCAGCGAAGAAGACAAATCACAAAC |
| | TGGTGTGAATAGAGCCAGCAAAGGAGGTCTTATCTATGGGAACTACCTGCATTTGGAA |
| | AAAGTTTTGAATGCACAAGAACTGCAAAGTGAAACAAAAGGAAATAAAATCCATGATG |
| | AACATCTTTTTATCATAACTCATCAAGCTTATGAACTCTGGTTTAAGCAAATCCTCTG |
| | GGAGTTGGATTCTGTTCGAGAGATCTTTCAGAATGGCCATGTCAGAGATGAAAGGAAC |
| | ATGCTTAAGGTTGTTTCTCGGATGCACCGAGTGTCAGTGATCCTGAAACTGCTGGTGC |
| | AGCAGTTTTCCATTCTGGAGACGATGACAGCCTTGGACTTCAATGACTTCAGAGAGTA |
| | CTTATCTCCAGCATCAGGCTTCCAGAGTTTGCAATTCCGACTATTAGAAAACAAGATA |
| | GGTGTTCTTCAGAACATGAGAGTCCCTTATAACAGAAGACATTATCGTGATAACTTCA |
| | AAGGAGAAGAAAATGAACTGCTACTTAAATCTGAGCAGGAAAAGACACTTCTGGAATT |
| | AGTGGAGGCATGGCTGGAAAGAACTCCAGGTTTAGAGTCACATGGATTTAACTTCTGG |
| | GGAAAGCTTGAAAAAAATATCACCAGAGGCCTGGAAGAGGAATTCATAAGGATTCAGG |
| | CTAAAGAAGAGTCTGAAGAAAAAGAGGAACAGGTGGCTGAATTTCAGAAGCAAAAAGA |
| | GGTGCTACTGTCCTTATTTGATGACAAACGTCATGAACATCTCCTTAGTAAAGGTGAA |
| | AGACGGCTGTCATACAGAGCACTTCAGGGAGCATTGATGATATATTTTTACAGGGAAG |
| | AGCCTAGGTTCCAGGTGCCTTTTCAGTTGCTGACTTCTCTTATGGACATAGATTCACT |
| | GATGACCAAATGGAGATATAACCATGTGTGCATGGTGCACAGAATGCTGGGCAGCAAA |
| | GCTGGCACCGGTGGTTCCTCAGGCTATCACTACCTGCGATCAACTGTGAGTGATAGGT |
| | ACAAGGTATTTGTAGATTTATTTAATCTTTCAACATACCTGATTCCCCGACACTGGAT |
| | ACCGAAGATGAACCCAACCATTCACAAATTTCTATATACAGCAGAATACTGTGATAGC |
| | TCCTACTTCAGCAGTGATGAATCAGATTAAAATCGTCTGCAAAATCTATGAAGAATAC |
| | TGGTTTCACAGCCTATTTAAGG |
| | ORF Start: ATG at 28 ORF Stop: TAA at 1246 |
| | SEQ ID NO: 438 406 aa MW at 47861.1kD |
| NOV58b, | MSGCPFLGNNFGYTFKKLPVEGSEEDKSQTGVNPASKGGLIYGNYLHLEKVLNAQELQ |
| CG97178-02 | |
| Protein Sequence | SETKGNKIHDEHLFITTHQAYELWFKQILWELDSVREIFQNGHVRDERNMLKVVSRMN |
| | RVSVILKLLVQQFSILETMTALDFNDFREYLSPASGFQSLQFRLLENKIGVLQNMRVP |
| | YNRRHYRDNFKGEENELLLKSEQEKTLLELVEAWLERTPGLESHGFNFWGKLEKNITR |
| | GLEEEFIRIQAKEESEEKEEQVAEFQKQKEVLLSLFDEKRHEHLLSKGERRLSYRALQ |
| | GALMIYFYREEPRFQVPFQLLTSLMDIDSLMTKWRYNHVCMVHRMLGSKAGTGGSSGY |
| | HYLRSTVSDRYKVFVDLFNLSTYLIPRHWIPKMNPTIHKFLYTAEYCDSSYFSSDESD |
| | SEQ ID NO: 439 1240 bp |
| NOV58c, | GCCGGATCCACCATGAGTGGGTGCCCATTTTTAGGAAACAACTTTGGATATACTTTTA |
| 275481043 | |
| DNA Sequence | AAAAACTCCCCGTAGAAGGCAGCGAAGAAGACAAATCACAAACTGGTGTGAATAGAGC |
| | CAGCAAAGGAGGTCTTATCTATGGGAACTACCTGCATTTGGAAAAAGTTTTGAATGCA |
| | CAAGAACTGCAAAGTGAAACAAAAGGAAATAAAATCCATGATGAACATCTTTTTATCA |
| | TAACTCATCAAGCTTATGAACTCTGGTTTAAGCAAATCCTCTGGGAGTTGGATTCTGT |
| | TCGAGAGATCTTTCAGAATGGCCATGTCAGAGATGAAAGGAACATGCTTAAGGTTGTT |
| | TCTCGGATGCACCGAGTGTCAGTGATCCTGAAACTGCTGGTGCAGCAGTTTTCCATTC |
| | TGGAGACGATGACAGCCTTGGACTTCAATGACTTCAGAGAGTACTTATCTCCAGCATC |
| | AGGCTTCCAGAGTTTGCAATTCCGACTATTAGAAAACAAGATAGGTGTTCTTCAGAAC |
| | ATGAGAGTCCCTTATAACAGAAGACATTATCGTGATAACTTCAAAGGAGAAGAAAATG |
| | AACTGCTACTTAAATCTGAGCAGGAAAAGACACTTCTGGAATTAGTGGAGGCATGGCT |
| | GGAAAGAACTCCAGGTTTAGAGTCACATGGATTTAACTTCTGGGGAAAGCTTGAAAAA |
| | AATATCACCAGAGGCCTGGAAGAGGAATTCATAAGGATTCAGGCTAAAGAAGAGTCTG |
| | AAGAAAAAGAGGAACACGTGGCTGAATTTCAGAAGCAAAAAGAGGTGCTACTGTCCTT |
| | ATTTGATGAGAAACGTCATGAACATCTCCTTAGTAAAGGTGAAAGACGGCTGTCATAC |
| | AGAGCACTTCAGGGAGCATTGATGATATATTTTTACAGGGAAGAGCCTAGGTTCCAGG |
| | TGCCTTTTCAGTTGCTGACTTCTCTTATGGACATAGATTCACTGATGACCAAATGGAG |
| | ATATAACCATGTGTCCATGGTGCACAGAATGCTGGGCAGCAAAGCTGGCACCGGTGGT |
| | TCCTCAGGCTATCACTACCTGCCATCAACTGTGAGTGATAGGTACAAGGTATTTGTAG |
| | ATTTATTTAATCTTTCAACATACCTGATTCCCCGACACTGGATACTGAAGATGAACCC |
| | AACCATTCACAAATTTCTATATACAGCAGAATACTGTGATAGCTCCTACTTCAGCAGT |
| | GATGAATCAGATGTCGACGCTG |
| | ORF Staff: at 1 ORF Stop: end of sequence |
| | SEQ ID NO: 440 414 aa MW at 48464.7kD |
| NOV58c, | AGSTMSGCPFLGNNFGYTFKKLPVEGSEEDKSQTGVNRASKGGLIYGNYLHLEKVLNA |
| 275481043 | |
| Protein Sequence | QELQSETKGNKTHDEHLFIITHQAYELWFKQILWELDSVREIFQNGHVRDERNNLKVV |
| | SRMHRVSVILKLLVQQFSILETMTALDFNDFREYLSPASGFQSLQFRLLENKIGVLQN |
| | MRVPYNRRHYRDNFKGEENELLLKSEQEKTLLELVEAWLERTPGLESHGFNFWGKLEK |
| | NITRGLEEEFIRIQAKEESEEKEEQVAEFQKQKEVLLSLFDEKRHEHLLSKGERRLSY |
| | RALQCALMIYFYREEPRFQVPFQLLTSLMDIDSLMTKWRYNHVCMVHRMLGSKAGTGG |
| | SSGYHYLRSTVSDRYKVFVDLFNLSTYLIPRHWILKMNPTIHKFLYTAEYCDSSYFSS |
| | DESDVDGX |

Sequence comparison of the above protein sequences yields the following sequence relationships shown in Table 58B.

TABLE 58B
Comparison of NOV58a against NOV58b and NOV58c.

| | | Identities/ |
|---|---|---|
| | | Similarities for |
| Protein | NOV58a Residues/ | the Matched |
| Sequence | Match Residues | Region |
| NOV58b | 1 . . . 406 | 405/406 (99%) |
| | 1 . . . 406 | 405/406 (99%) |
| NOV58c | 1 . . . 406 | 404/406 (99%) |
| | 5 . . . 410 | 404/406 (99%) |

Further analysis of the NOV58a protein yielded the following properties shown in Table 58C.

TABLE 58C
Protein Sequence Properties NOV58a

| | |
|---|---|
| PSort | 0.5095 probability located in microbody (peroxisome); |
| analysis: | 0.4500 probability located in cytoplasm; 0.1000 |
| | probability located in mitochondrial matrix space; |
| | 0.1000 probability located in lysosome (lumen) |
| SignalP | No Known Signal Sequence Predicted |
| analysis: | |

A search of the NOV58a protein against the Geneseq database, a proprietary database that contains sequences published in patents and patent publication, yielded several homologous proteins shown in Table 58D.

TABLE 58D
Geneseq Results for NOV58a

| | | | Identities/ | |
|---|---|---|---|---|
| | | | Similarities for | |
| Geneseq | Protein/Organism/Length | NOV58a Residues/ | the Matched | Expect |
| Identifier | [Patent #, Date] | Match Residues | Region | Value |
| AAR21549 | Human Tryptophan Oxygenase | 1 . . . 406 | 403/406 (99%) | 0.0 |
| | TDO2 - Homo sapiens, 406 aa. | 1 . . . 406 | 404/406 (99%) | |
| | [WO9202637-A, 20 FEB. 1992] | | | |
| AAR21547 | Human Tryptophan-2,3-dioxygenase | 1 . . . 396 | 365/396 (92%) | 0.0 |
| | deduced from clone HTO3 - Homo | 1 . . . 394 | 369/396 (93%) | |
| | sapiens, 436 aa. [WO9202637-A, | | | |
| | 20 FEB. 1992] | | | |
| AAR21546 | Human Tryptophan-2,3-dioxygenase | 1 . . . 228 | 225/228 (98%) | e−130 |
| | deduced from clone HTO3 - Homo | 1 . . . 228 | 226/228 (98%) | |
| | sapiens, 238 aa. [WO9202637-A, | | | |
| | 20 FEB. 1992] | | | |
| ABB58903 | Drosophila melanogaster | 19 . . . 389 | 213/373 (57%) | e−115 |
| | polypeptide SEQ ID NO 3501 - | 4 . . . 374 | 273/373 (73%) | |
| | Drosophila melanogaster, 379 aa. | | | |
| | [WO200171042-A2, 27 SEP. 2001] | | | |
| AAU11269 | Human N-acetyltransferase 1 | 132 . . . 223 | 32/96 (33%) | 0.44 |
| | (NAT1) variant polypeptide - Homo | 194 . . . 288 | 44/96 (45%) | |
| | sapiens, 290 aa. [WO200179551- | | | |
| | A1, 25 OCT. 2001] | | | |

In a BLAST search of public sequence datbases, the NOV58a protein was found to have homology to the proteins shown in the BLASTP data in Table 58E.

TABLE 58E
Public BLASTP Results for NOV58a

| | | | Identities/ | |
|---|---|---|---|---|
| Protein | | | Similarities for | |
| Accession | | NOV58a Residues/ | the Matched | Expect |
| Number | Protein/Organism/Length | Match Residues | Portion | Value |
| P48775 | Tryptophan 2,3-dioxygenase (EC | 1 . . . 406 | 406/406 (100%) | 0.0 |
| | 1.13.11.11) (Tryptophan pyrrolase) | 1 . . . 406 | 406/406 (100%) | |
| | (Tryptophanase) (Tryptophan | | | |
| | oxygenase) (Tryptamin 2,3- | | | |
| | dioxygenase) (TRPO) - Homo | | | |
| | sapiens (Human), 406 aa. | | | |
| Q8VCW3 | Tryptophan 2,3-dioxygenase - Mus | 1 . . . 406 | 360/406 (88%) | 0.0 |
| | musculus (Mouse), 406 aa. | 1 . . . 406 | 388/406 (94%) | |
| P48776 | Tryptophan 2,3-dioxygenase (EC | 1 . . . 406 | 359/406 (88%) | 0.0 |
| | 1.13.11.11) (Tryptophan pyrrolase) | 1 . . . 406 | 388/406 (95%) | |
| | (Tryptophanase) (Tryptophan | | | |
| | oxygenase) (Tryptamin 2,3- | | | |
| | dioxygenase) (TRPO) - Mus | | | |
| | musculus (Mouse), 406 aa. | | | |
| P21643 | Tryptophan 2,3-dioxygenase (EC | 1 . . . 406 | 360/406 (88%) | 0.0 |
| | 1.13.11.11) (Tryptophan pyrrolase) | 1 . . . 406 | 389/406 (95%) | |
| | (Tryptophanase) (Tryptophan | | | |
| | oxygenase) (Tryptamin 2,3- | | | |
| | dioxygenase) (TRPO) - Rattus | | | |
| | norvegicus (Rat), 406 aa. | | | |
| O17440 | VERMILION - Drosophila | 19 . . . 389 | 214/374 (57%) | e−115 |
| | ananassae (Fruit fly), 380 aa. | 4 . . . 375 | 275/374 (73%) | |

PFam analysis predicts that the NOV58a protein contains the domains shown in the Table 58F.

TABLE 58F
Domain Analysis of NOV58a

| | | | |
|---|---|---|---|
| | | Identities/ | |
| | | Similarities for | |
| Pfam | NOV58a | the Matched | Expect |
| Domain | Match Region | Region | Value |

No Significant Matches Found

### EXAMPLE 59

The NOV59 clone was analyzed, and the nucleotide and encoded polypeptide sequences are shown in Table 59A.

TABLE 59A
NOV59 Sequence Analysis

| | |
|---|---|
| | SEQ ID NO: 441 1060 bp |
| NOV59a, | CGCGGGCCGACTGGTGTTTATCCGTCACTCGCCCAGGTTCCTTGGGTCATGGTGCCAG |
| CG98102-01 | |
| DNA Sequence | CCTGACTGAGAAGAGGACGCTCCCGGGAGACGAATGAGGAACCACCTCCTCCTACTGT |
| | TCAAGTACAGGGGCCTGGTCCGCAAAGGGAAGAAAAGCAAAAGACGAAAATGGCTAAA |
| | TTCGTGATCCGCCCAGCCACTGCCGCCGACTGCAGTGACATACTGCGGCTGATCAAGG |
| | AGCTGGCTAAATATGAATACATGGAAGAACAAGTAATCTTAACTGAAAAAGATCTGCT |
| | AGAAGATGGTTTTGGAGACCACCCCTTTTACCACTGCCTGGTTGCAGAAGTGCCGAAA |
| | GAGCACTGGACTCCGGAAGGACACAGCATTGTTGGTTTTGCCATGTACTATTTTACCT |
| | ATGACCCGTGGATTGGCAAGTTATTGTATCTTGAGGACTTCTTCGTGATGAGTGATTA |
| | TAGAGGCTTTGGCATAGGATCAGAAATTCTGAAGAATCTAAGCCAGGTTGCAATGAGG |
| | TGTCGCTGCAGCAGCATGCACTTCTTGGTAGCAGAATGGAATGAACCATCCATCAACT |
| | TCTATAAAAGAAGAGGTGCTTCTGATCTGTCCAGTGAAGAGGGTTGGAGACTGTTCAA |
| | GATCGACAAGGAGTACTTGCTAAAAATGGCAACAGAGGAGTGAGGAGTGCTGCTGTAG |
| | ATGACAACCTCCATTCTATTTTAGAATAAATTCCCAACTTCTCTTGCTTTCTATGCTG |
| | TTTGTAGTGAAATAATAGAATGAGCACCCATTCCAAAGCTTTATTACCAGTGGCGTTG |
| | TTGCATGTTTGAAATGAGGTCTGTTTAAAGTGGCAATCTCAGATGCAGTTTGGAGAGT |
| | CAGATCTTTCTCCTTGAATATCTTTCGATAAACAACAAGGTGGTGTGATCTTAATATA |
| | TTTGAAAAAAACTTCATTCTCGTGAGTCATTTAAATGTGTACAATGTACACACTGGTA |
| | CTTAGAGTTTCTGTTTGATTCTTTTTTAATAAACTACTCTTTGATTTAAAAAAAAAAA |
| | AAAAAAAAAAAAAAAA |
| | ORF Start: ATG at 166 ORF Stop: TGA at 679 |
| | SEQ ID NO: 442 171 aa MW at 20023.8kD |
| NOV59a, | MAKFVIRPATAADCSDILRLIKELAKYEYMEEQVILTEKDLLEDGFGEHPFYHCLVAE |
| CG98102-01 | |
| Protein Sequence | VPKEHWTPEGHSIVGFAMYYFTYDPWIGKLLYLEDFFVMSDYRGFGIGSEILKNLSQV |
| | AMRCRCSSMHFLVAEWNEPSINFYKRROASDLSSEEGWRLFKIDKBYLLKMATEE |
| | SEQ ID NO: 443 1052 bp |
| NOV59b, | CGGCCGCGTCGACCGCGGGCTGACTGGTTTATCCGTCACTCGCCGAGGTTCCTTGG |
| CG98102-03 | |
| DNA Sequence | GTCATGGTGCCAGCCTGACTGAGAAGAGGACGCTCCCGGGAGACGAATGAGGAACCAC |
| | CTCCTCCTACTGTTCAAGTACAGGGGCCTCGTCCGCAAAGGGAAGAAAAGCAAAAGAC |
| | GAAAATGGCTAAATTCGTGATCCGCCCAGCCACTGCCGCCGACTGCAGTGACATACTG |
| | CGGCTGATCAGGAGCTGGCTAAATATGAATACATGGAGAACAAGTAATCTTAACTG |
| | AAAAAGATCTGCTAGAAGATGGTTTTGGAGAGCACCCCTTTTACCACTGCCTGGTTGC |
| | AGAAGTGCCGAAAGAGCACTGGACTCCGGAAGGACACAGCATTGTTGGTTTTGCCATG |
| | TACTATTTTACCTATGACCCGTGGATTGGCAAGTTATTGTATCTTGAGGACTTTTTCG |
| | TGATGAGTGATTATAGAGGCTTTGGCATAGGATCAGAAATTCTGAAGAATCTAAGCCA |
| | GGTTGCAATGAGGTGTCGCTGCAGCAGCATGCACTTCTTGGTAGCAGAATGGAATGAA |
| | CCATCCATCAACTTCTATAAAAGAAGAGGTGCTTCTGATCTGTCCAGTGAAGAGGGTT |
| | GGAGACTGTTCAAGATCGACAAGGAGTACTTGCTAAAAATGGCAACAGAGGAGTGAGG |
| | AGTGCTGCTGTAGATGACAACCTCCATTCTATTTTAGAATAAATTCCCAACTTCTCTT |
| | GCTTTCTATGCTGTTTGTAGTGAAATAATAGAATGAGCACCCATTCCAAAGCTTTATT |
| | ACCAGTGGCGTTGTTGCATGTTTGAAATGAGGTCTGTTTAAAGTGGCAATCTCAGATG |
| | CAGTTTGGAGAGTCAGATCTTTCTCCTTGAATATCTTTCGATAAACAACAAGGTGGTG |
| | TGATCTTAATATATTTGAAAAAAACTTCATTCTCGTGAGTCATTTAAATGTGTACAAT |
| | GTACACACTGGTACTTAGAGTTTCTGTTTGATTCTTTTTTAATAAACTACTCTTTGAT |
| | TTAAAAAA |
| | ORF Start: ATG at 179 ORF Stop: IGA at 692 |
| | SEQ ID NO: 444 171 aa MW at 20023.8kD |
| NOV59b, | MAKFVIRPATAADCSDILRLTKELAKYEYMEEQVILTEKDLLEDGFGEHPFYHCLVAE |
| CG98102-03 | |
| Protein Sequence | VPKEHWTPEGHSIVGFAMYYFTYDPWIGKLLYLEDFFVMSDYRGFGIGSEILKNLSQV |
| | AMRCRCSSMHFLVAEWNEPSINFYKRRGASDLSSEEGWRLFKIDKEYLLKMATEE |
| | SEQ ID NO: 445 665 bp |
| NOV59c, | ACCTCCTCCTACTGTTCAACTACAGGGGCCTGGTCCGCAAAGGGAAGAAAAGCAAAAG |
| CG98102-02 | |
| DNA Sequence | ACGAAAATGGCTAAATTCGTGATCCGCCCAGCCACTGCCGCCGACTGCAGTGACATAC |
| | TGCGGCTGATCAAGGAGCTGGCTAAATATGAATACATGGAAGAACAAGTAATCTTAAC |
| | TGAAAAAGATCTGCTAGAAGATGGTTTTGGAGAGCACCCCTTTTACCACTGCCTGGTT |
| | GCAGAAGTGCCGAAAGAGCACTGGACTCCGGAAGGACACAGCATTGTTGGTTTTGCCA |
| | TGTACTATTTTACCTATGACCCGTGGATTGGCAAGTTATTGTATCTTGAGGACTTCTT |
| | CGTGATGAGTGATTATAGAGGCTTTGGCATAGCATCAGAAATTCTGAAGAATCTAAGC |
| | CAGGTTGCAATGAGGTGTCGCTGCAGCAGCATGCACTTCTTGGTAGCAGAATGGAATG |
| | AACCATCCATCAACTTCTATAAAAGAAGAGGTGCTTCTGATCTGTCCAGTGAAGAGGG |
| | TTGGAGACTGTTCAAGATCGACAAGGAGTACTTGCTAAAAATGGCAACAGAGGAGTGA |
| | GGAGTGCTGCTGTAGATGACAACCTCCATTCTATTTTAGAATAAATTCCCAACTTCTC |
| | TTGCTTTCTATGCTGTTTGTAGTGAAA |
| | ORF Start: ATG at 65 ORF Stop: TGA at 578 |
| | SEQ ID NO: 446 171 aa MW at 20023.8kD |
| NOV59c, | MAKFVIRPATAADCSDILRLIKELAKYEYMEEQVILTEKDLLEDGFGEHPFYHCLVAE |
| CG98102-02 | |
| Protein Sequence | VPKEHWTPEGHSIVGFAMYYFTYDPWIGKLLYLEDFFVMSDYRGFGIGSEILKNLSQV |
| | AMRCRCSSMHFLVAEWNEPSINFYKRRGASDLSSEEGWRLFKIDKEYLLKMATEE |
| | SEQ ID NO: 447 596 bp |
| NOV59d, | ACCTCCTCCTACTGTTCAAGTACAGGGGCCTGGTCCGCAAAGGGAAGAAAAGCAAAAG |
| CG98102-04 | |
| DNA Sequence | ACGAAAATGGCTAAATATGAATACATGGAAGAACAAGTAATCTTAACTGAAAAAGATC |
| | TGCTAGAAGATGGTTTTGGAGAGCACCCCTTTTACCACTGCCTGGTTGCAGAAGTGCC |
| | GAAAGAGCACTGGACTCCGGAAGGACACAGCATTCTTGGTTTTGCCATGTACTATTTT |
| | ACCTATGACCCGTGGATTGGCAAGTTATTGTATCTTGACGACTTCTTCGTGATGAGTG |
| | ATTATAGAGGCTTTGGCATAGGATCAGAAATTCTGAAGAATCTAAGCCAGGTTGCAAT |
| | GAGGTGTCGCTGCAGCAGCATGCACTTCTTGGTAGCAGAATGGAATGAACCATCCATC |
| | AACTTCTATAAAGAAGAGGTGCTTCTGATCTGTCCAGTGAAGAGGGTTGGAGACTGT |
| | TCAAGATCGACAAGGAGTACTTGCTAAAAATGGCAACAGAGGAGTGAGGAGTGCTGCT |
| | GTAGATGACAACCTCCATTCTATTTTAGAATAAATTCCCAACTTCTCTTGCTTTCTAT |
| | GCTGTTTGTAGTGAAA |
| | ORF Start: ATG at 65 ORF Stop: TGA at 509 |
| | SEQ ID NO: 448 148 aa MW at 17497.8kD |
| NOV59d, | MAKYEYMEEQVILTEKDLLEDGFGEHPFYHCLVAEVPKEHWTPEGHSIVGFAMYYFTY |
| CG98102-04 | |
| Protein Sequence | DPWIGKLLYLEDFFVMSDYRGFGIGSEILKNLSQVAMRCRCSSMHFLVAEWNEPSINF |
| | YKRRGASDLSSEEGWRLFKIDKEYLLKMATEE |
| | SEQ ID NO: 449 1157 bp |
| NOV59e, | CTGGTGTTTATCCGTCACTCGCCGAGGTTCCTTGGGTCATGGTGCCAGCCTGACTGAG |
| CG98102-05 | |
| DNA Sequence | AAGAGGACGCTCCCGGGAGACGAATGAGGAACCACCTCCTCCTACTGTTCAAGTACAG |
| | GGGCCTGGTCCGCAAAGGGAAGAAAAGCAAAAGACGAAAATCGCTAAATTCGTGATCC |
| | GCCCAGCCACTGCCGCCGACTGCAGTGACATACTGCGGCTGATCAAGGAGCTGGCTAA |
| | ATATGAATACATGGAAGAACAAGTAATCTTAACTGAAAAAGATCTGCTAGAAGATGGT |
| | TTTGGAGAGCACCCCTTTTACCACTGCCTGGTTGCAGAAGTGCCGAAAGAGCACTGGA |
| | CTCCGGAAGGTTACAGTCTCTAGCTTCGCCATGTACATGGCCCTTCCGTGTACATGGA |
| | TGGGCGGGGAGGTAACTAAAGATCCTTTACACAATAAAAGTAGATGATCATGATAAAT |
| | GAGGACACACCATTGTTGGTTTTGCCATGTACTATTTTACCTATGACCCGTGGATTGG |
| | CAAGTTATTGTATCTTGAGGACTTCTTCGTGATGAGTGATTATAGAGGCTTTGGCATA |
| | GGATCAGAAATTCTGAAGAATCTAAGCCAGGTTGCAATGAGGTGTCGCTGCAGCAGCA |
| | TGCACTTCTTGGTAGCAGAATGGAATGAACCATCCATCAACTTCTATAAAAGAAGAGG |
| | TCCTTCTGATCTGTCCAGTGAAGAGGGTTGGAGACTGTTCAAGATCGACAAGGAGTAC |
| | TTGCTAAAAATGGCAACAGAGGAGTGAGGAGTGCTGCTGTAGATGACAACCTCCATTC |
| | TATTTTAGAATAATTCCCAACTTCTCTTGCTTTCTATGCTGTTTGTAGTGAAATAAT |
| | AGAATGACCACCCATTCCAAAGCTTTATTACCAGTGGCGTTGTTGCATGTTTGAAATG |
| | AGGTCTGTTTAAAGTGGCAATCTCAGATGCAGTTTGGAGAGTCAGATCTTTCTCCTTG |
| | AATATCTTTCGATAAACAACAAGGTGGTGTGATCTTAATATATTTGAAAAAAACTTCA |
| | TTCTCGTGAGTCATTTAAATGTGTACAATGTACACACTGGTACTTAGAGTTTCTGTTT |
| | GATTCTTTTTTAATAAACTACTCTTTGATTTAAAAAAAAAAAAAAAAAAAAAAAA |
| | ORF Start: ATG at 491 ORF Stop: TGA at 779 |
| | SEQ ID NO: 450 96 aa MW at 11464.0kD |
| NOV59e, | MYYFTYDPWIGKLLYLEDFFVMSDYRGFGIGSEILKNLSQVAMRCRCSSMHFLVAEWN |
| CG98102-05 | |
| Protein Sequence | EPSINFYKRRGASDLSSEEGWRLFKIDKEYLLKIVIATEE |
| | SEQ ID NO: 451 1107 bp |
| NOV59f, | TGGAATTCGGCCATACTGGGCGGTAGCGCAGCTCTTAGTCGCGGGCCGACTGGTGTTT |
| CG98102-06 | |
| DNA Sequence | ATCCGTCACTCGCCGAGGTTCCTTGGGTCATGGTGCCAGCCTGACTGAGAAGAGGACG |
| | CTCCCGGGAGACGAATGAGTGAACCACCTCCTCCTACTGTTCAAGTACAGGGGCCTGG |
| | TCCGCAAAGGGAAGAAAAGCAAAAGACGAAAATGGCTAAATTCGTGATCCGCCCAGCC |
| | ACTGCCGCCGACTGCAGTGACATACTGCGGCTGATCAAGGAGCTGGCTAAATATGAAT |
| | ACATGGAAGAACAAGTAATCTTAACTGAAAAAGATCTGCTAGAAGATGGTTTTGGAGA |
| | GCACCCCTTTTACCACTGCCTGGTTGCAGAAGTGCCGAAAGAGCACTGGACTCCGGAA |
| | GGTAACCCCTCGCCCTTGTCCAGGGTAAGCCATGTAGTAGTTTACCTATACCCGTGTT |
| | ATGTAAGCAAGTTATCGTGTCTTGAGGACTTCTTCGTGATGAGTGATTACTCGAGGCT |
| | TTGGCATAGGATCAGAAATTCTGAAGAATCTAAGCCAGGTTGCAATGAGGTGTCGCTG |
| | CCAGCAGCATGCACTTCTTGGGTAGCAGAATGGAATGAACCATCCATCAACTTCTATA |
| | AAAGAAGAGGTGCTTCTGATCTGTCCAGTGAAGAGGGTTGGAGATGTTCAGATCGCAA |
| | GGAGTACTGCTAAAAATGGCAACAGGGAGTACCAGACTGTGCTGATAGATGACAACCT |
| | CCATTCTATTTTAGAATAAATTCCCAACTTCTCTTGCTTTCTATGCTGTTTGTAGTGA |
| | AATAATAGAATGAGCACCCATTCCAAAGCTTTATTACCAGTGGCGTTGTTGCATGTTT |
| | GAAATGAGGTCTGTTTAAAGTGGCAATCTCAGATGCAGTTTGGAGAGTCAGATCTTTC |
| | TCCTTGAATATCTTTCGATAAACAACAAGGTGGTGTGATCTTAATATATTTGAAAAAA |
| | ACTTCATTCTCGTGAGTCATTTAAATGTGTACAATGTACACACTGGTACTTAGAGTTT |
| | CTGTTTGATTCTTTTTTAATAAACTACTCTTTGATTTAAAAAAAAAAAAAAAAAAAAA |
| | AAAAA |
| | ORF Start: ATG at 131 ORF Stop: TAA at 707 |
| | SEQ ID NO: 452 192 aa MW at 22209.9kD |
| NOV59f, | MSEPPPPTVQVQGPGPQREEKQKTKMAKFVIRPATAADCSDILRLIKELAKYEMEEQ |
| CG98102-06 | |
| Protein Sequence | VILTEKDLLEDGFGEHPFYHCLVAEVPKEHWTPEGNPSPLSRVSHVVVYLYPCYVSKL |
| | WCLEDFFVMSDYSRLWHRIRNSEESKPGCNEVSLPAACTSWVAEWNEPSINFYKRRGA |
| | SDLSSEEGWRCSDRKEYC |

Sequence comparison of the above protein sequences yields the following sequence relationships shown in Table 59B.

TABLE 59B
Comparison of NOV59a against NOV59b through NOV59f.

| | | Identities/ |
|---|---|---|
| | | Similarities for |
| Protein | NOV59a Residues/ | the Matched |
| Sequence | Match Residues | Region |
| NOV59b | 1 . . . 171 | 171/171 (100%) |
| | 1 . . . 171 | 171/171 (100%) |
| NOV59c | 1 . . . 171 | 171/171 (100%) |
| | 1 . . . 171 | 171/171 (100%) |
| NOV59d | 24 . . . 171 | 147/148 (99%) |
| | 1 . . . 148 | 148/148 (99%) |
| NOV59e | 76 . . . 171 | 96/96 (100%) |
| | 1 . . . 96 | 96/96 (100%) |
| NOV59f | 1 . . . 155 | 115/163 (70%) |
| | 26 . . . 184 | 124/163 (75%) |

Further analysis of the NOV59a protein yielded the following properties shown in Table 59C.

TABLE 59C
Protein Sequence Properties NOV59a

| | |
|---|---|
| PSort | 0.6400 probability located in microbody (peroxisome); |
| analysis: | 0.6153 probability located in mitochondrial matrix |
| | space; 0.3177 probability located in mitochondrial |
| | inner membrane; 0.3177 probability located in |
| | mitochondrial intermembrane space |
| SignalP | No Known Signal Sequence Predicted |
| analysis: | |

A search of the NOV59a protein against the Geneseq database, a proprietary database that contains sequences published in patents and patent publication, yielded several homologous proteins shown in Table 59D.

TABLE 59D
Geneseq Results for NOV59a

| | | | Identities/ | |
|---|---|---|---|---|
| | | | Similarities for | |
| Geneseq | Protein/Organism/Length | NOV59a Residues/ | the Matched | Expect |
| Identifier | [Patent #, Date] | Match Residues | Region | Value |
| ABB57094 | Mouse ischaemic condition related | 1 . . . 171 | 165/171 (96%) | 1e−96 |
| | protein sequence SEQ ID NO: 207 - | 1 . . . 171 | 168/171 (97%) | |
| | Mus musculus, 171 aa. | | | |
| | [WO200188188-A2, 22 NOV. | | | |
| | 2001] | | | |
| AAU30048 | Novel human secreted | 1 . . . 158 | 146/161 (90%) | 9e−81 |
| | protein #539 - Homo sapiens, 218 aa. | 35 . . . 195 | 151/161 (93%) | |
| | [WO200179449-A2, 25 OCT. 2001] | | | |
| AAB82049 | Human spermidine/spermine acetyl | 1 . . . 155 | 115/163 (70%) | 4e−56 |
| | transferase protein isomer - Homo | 26 . . . 184 | 124/163 (75%) | |
| | sapiens, 192 aa. [CN1278003-A, 27 | | | |
| | DEC. 2000] | | | |
| AAB44145 | Human cancer associated protein | 42 . . . 127 | 85/86 (98%) | 3e−48 |
| | sequence SEQ ID NO: 1590 - Homo | 1 . . . 86 | 85/86 (98%) | |
| | sapiens, 92 aa. [WO200055350-A1, | | | |
| | 21 SEP. 2000] | | | |
| AAW58394 | Human spermidine/spermine N1- | 1 . . . 168 | 78/168 (46%) | 9e−41 |
| | acetyltransferase - Homo sapiens, | 1 . . . 168 | 109/168 (64%) | |
| | 170 aa. [WO9818938-A1, 07 MAY | | | |
| | 1998] | | | |

In a BLAST search of public sequence datbases, the NOV59a protein was found to have homology to the proteins shown in the BLASTP data in Table 59E.

TABLE 59E
Public BLASTP Results for NOV59a

| | | | Identities/ | |
|---|---|---|---|---|
| | | NOV59a | Similarities | |
| Protein | | Residues/ | for the | |
| Accession | | Match | Matched | Expect |
| Number | Protein/Organism/Length | Residues | Portion | Value |
| P21673 | Diamine acetyltransferase (EC | 1 . . . 171 | 171/171 (100%) | 3e−99 |
| | 2.3.1.57) (Spermidine/spermine | 1 . . . 171 | 171/171 (100%) | |
| | N(1)- acetyltransferase) (SSAT) | | | |
| | (Putrescine acetyltransferase) - | | | |
| | Homo sapiens (Human), 171 aa. | | | |
| JH0783 | diamine N-acetyltransferase (EC | 1 . . . 171 | 170/171 (99%) | 1e−98 |
| | 2.3.1.57) - human, 171 aa. | 1 . . . 171 | 171/171 (99%) | |
| P49431 | Spermidine/spermine N(1)- | 1 . . . 171 | 166/171 (97%) | 7e−97 |
| | acetyltransferase (EC 2.3.1.57) | 1 . . . 171 | 169/171 (98%) | |
| | (Diamine acetyltransferase) (SSAT) | | | |
| | (Putrescine acetyltransferase) - Mus | | | |
| | saxicola (Spiny mouse), 171 aa. | | | |
| Q28999 | Diamine acetyltransferase (EC | 1 . . . 171 | 168/171 (98%) | 1e−96 |
| | 2.3.1.57) (Spermidine/spermine | 1 . . . 171 | 169/171 (98%) | |
| | N(1)- acetyltransferase) (SSAT) | | | |
| | (Putrescine acetyltransferase) - Sus | | | |
| | scrofa (Pig), 171 aa. | | | |
| Q9JHW6 | Spermidine/spermine N1- | 1 . . . 171 | 164/171 (95%) | 2e−96 |
| | acetyltransferase - Cricetulus | 1 . . . 171 | 169/171 (97%) | |
| | griseus (Chinese hamster), 171 aa. | | | |

PFam analysis predicts that the NOV59a protein contains the domains shown in the Table 59F.

TABLE 59F
Domain Analysis of NOV59a

| | | Identities/ | |
|---|---|---|---|
| | NOV59a | Similarities | |
| | Match | for the | |
| Pfam Domain | Region | Matched Region | Expect Value |
| Acetyltransf | 63 . . . 146 | 23/85 (27%) | 1.6e−16 |
| | | 59/85 (69%) | |

### Example B

Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, CA) were then transferred from E.coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune/autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°−60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_Screening Panel_v1.4, v1.5, v1.6 and 1.7

The plates for Panels 1.4, 1.5, 1.6 and 1.7 include 2 control wells (genomic DNA control and chemistry control) and 94 wells containing cDNA from various samples. The samples in Panels 1.4, 1.5, 1.6 and 1.7 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4, 1.5, and 1.6 are widely available through the American Type Culture Collection (ATCC), a repository for cultured cell lines, and were cultured using the conditions recommended by the ATCC. The normal tissues found on Panels 1.4, 1.5, 1.6, 1.7 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D, 2.2, 2.3 and 2.4

The plates for Panels 2D, 2.2, 2.3 and 2.4 generally include two control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI) or from Ardais or Clinomics. The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI/ CHTN/Ardais/Clinomics). Unmatched RNA samples from tissues without malignancy (normal tissues) were also obtained from Ardais or Clinomics. This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen. General oncology screening panel_v_{—}2.4 is an updated version of Panel 2D.

HASS Panel v 1.0

The HASS panel v 1.0 plates are comprised of 93 cDNA samples and two controls. Specifically, 81 of these samples are derived from cultured human cancer cell lines that had been subjected to serum starvation, acidosis and anoxia for different time periods as well as controls for these treatments, 3 samples of human primary cells, 9 samples of malignant brain cancer (4 medulloblastomas and 5 glioblastomas) and 2 controls. The human cancer cell lines are obtained from ATCC (American Type Culture Collection) and fall into the following tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

ARDAIS Panel v 1.0

The plates for ARDAIS panel v 1.0 generally include 2 control wells and 22 test samples composed of RNA isolated from human tissue procured by surgeons working in close cooperation with Ardais Corporation. The tissues are derived from human lung malignancies (lung adenocarcinoma or lung squamous cell carcinoma) and in cases where indicated many malignant samples have “matched margins” obtained from noncancerous lung tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). Unmatched malignant and non-malignant RNA samples from lungs were also obtained from Ardais. Additional information from Ardais provides a gross histopathological assessment of tumor differentiation grade and stage. Moreover, most samples include the original surgical pathology report that provides information regarding the clinical state of the patient.

Panel 3D, 3.1 and 3.2

The plates of Panel 3D, 3.1, and 3.2 are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D, 3.1, 3.2, 1, 1.1., 1.2, 1.3D, 1.4, 1.5, and 1.6 are of the most common cell lines used in the scientific literature.

AI.05 Chondrosarcoma

The AI.05 chondrosarcoma plates are comprised of SW1353 cells that had been subjected to serum starvation and treatment with cytokines that are known to induce MMP (1, 3 and 13) synthesis (eg. IL1beta). These treatments include: IL-1beta (10 ng/ml), IL-1beta+TNF-alpha (50 ng/ml), IL-1beta+Oncostatin (50 ng/ml) and PMA (100 ng/ml). The SW1353 cells were obtained from the ATCC (American Type Culture Collection) and were all cultured under standard recommended conditions. The SW1353 cells were plated at 3×10⁵ cells/ml (in DMEM medium-10 % FBS) in 6-well plates. The treatment was done in triplicate, for 6 and 18 h. The supernatants were collected for analysis of MMP 1, 3 and 13 production and for RNA extraction. RNA was prepared from these samples using the standard procedures.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1 % serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, UT), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 μg/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μm non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for Al_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

−M=Male

−F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; patient not demented but showing sever AD-like pathology

SupTemporal Ctx=Superior Temporal Cortex

Inf Temporal Ctx=Inferior Temporal Cortex

A. CG101683-01: COT.

Expression of gene CG101683-01 was assessed using the primer-probe sets Ag3116, Ag3551 and Ag4828, described in Tables AA, AB and AC. Results of the RTQ-PCR runs are shown in Tables AD, AE, AF, AG, AH, AI and AJ.

TABLE AA
Probe Name Ag3116

| | | | Start | SEQ ID |
|---|---|---|---|---|
| Primers | Sequences | Length | Position | No |
| Forward | 5′-catgttctcaagggacttgatt-3′ | 22 | 1072 | 453 |
| Probe | TET-5′-cactcaaagaagtgatccatcatga-3′-TAMRA | 26 | 1099 | 454 |
| Reverse | 5′-ttttgtggacatgaaaacaatg-3′ | 22 | 1140 | 455 |

TABLE AB
Probe Name Ag3551

| | | | Start | SEQ ID |
|---|---|---|---|---|
| Primers | Sequences | Length | Position | No |
| Forward | 5-′-catgttctcaagggacttgatt-3′ | 22 | 1072 | 456 |
| Probe | TET-5′-cactcaaagaaagtgatccatcatga-3′-TAMRA | 26 | 1099 | 457 |
| Reverse | 5′-ttttgtggacatgaaaacaatg-3′ | 22 | 1140 | 458 |

TABLE AC
Probe Name Ag4828

| | | | Start | SEQ ID |
|---|---|---|---|---|
| Primers | Sequences | Length | Position | No |
| Forward | 5′-gaggaatctgagatgctcaaga-3′ | 22 | 1663 | 459 |
| Probe | TET-5′-caacgctctctacatcgacctcgg-3′-TAMRA | 26 | 1687 | 460 |
| Reverse | 5′-tccccgaacaagattgaagt-3′ | 20 | 1727 | 461 |

TABLE AD
CNS_neurodegeneration_v1.0

| | Rel. Exp. (%) |
|---|---|
| | Ag3551, Run |
| Tissue Name | 209990366 |
| AD 1 Hippo | 20.0 |
| AD 2 Hippo | 44.1 |
| AD 3 Hippo | 7.1 |
| AD 4 Hippo | 5.6 |
| AD 5 hippo | 100.0 |
| AD 6 Hippo | 57.0 |
| Control 2 Hippo | 24.7 |
| Control 4 Hippo | 51.4 |
| Control (Path) 3 Hippo | 48.6 |
| AD 1 Temporal Ctx | 21.3 |
| AD 2 Temporal Ctx | 39.5 |
| AD 3 Temporal Ctx | 6.1 |
| AD 4 Temporal Ctx | 16.8 |
| AD 5 Inf Temporal Ctx | 100.0 |
| AD 5 Sup Temporal Ctx | 91.4 |
| AD 6 Inf Temporal Ctx | 58.2 |
| AD 6 Sup Temporal Ctx | 65.5 |
| Control 1 Temporal Ctx | 20.3 |
| Control 2 Temporal Ctx | 21.2 |
| Control 3 Temporal Ctx | 10.8 |
| Control 4 Temporal Ctx | 6.9 |
| Control (Path) 1 Temporal Ctx | 42.0 |
| Control (Path) 2 Temporal Ctx | 26.4 |
| Control (Path) 3 Temporal Ctx | 14.6 |
| Control (Path) 4 Temporal Ctx | 18.8 |
| AD 1 Occipital Ctx | 13.5 |
| AD 2 Occipital Ctx (Missing) | 0.0 |
| AD 3 Occipital Ctx | 4.0 |
| AD 4 Occipital Ctx | 15.8 |
| AD 5 Occipital Ctx | 34.6 |
| AD 6 Occipital Ctx | 46.0 |
| Control 1 Occipital Ctx | 21.0 |
| Control 2 Occipital Ctx | 41.5 |
| Control 3 Occipital Ctx | 16.3 |
| Control 4 Occipital Ctx | 13.0 |
| Control (Path) 1 Occipital Ctx | 95.3 |
| Control (Path) 2 Occipital Ctx | 10.2 |
| Control (Path) 3 Occipital Ctx | 21.5 |
| Control (Path) 4 Occipital Ctx | 24.0 |
| Control 1 Parietal Ctx | 17.2 |
| Control 2 Parietal Ctx | 57.4 |
| Control 3 Parietal Ctx | 16.5 |
| Control (Path) 1 Parietal Ctx | 28.3 |
| Control (Path) 2 Parietal Ctx | 15.8 |
| Control (Path) 3 Parietal Ctx | 19.6 |
| Control (Path) 4 Parietal Ctx | 61.1 |

TABLE AE
General screening_panel_v1.4

| | Rel. | Rel. | Rel. |
|---|---|---|---|
| | Exp. (%) | Exp. (%) | Exp. (%) |
| | Ag3116, | Ag3551, | Ag4828, |
| Tissue | Run | Run | Run |
| Name | 219923407 | 218328114 | 217081802 |
| Adipose | 100.0 | 58.2 | 53.6 |
| Melanoma* Hs688(A).T | 18.8 | 9.0 | 15.5 |
| Melanoma* Hs688(B).T | 21.3 | 10.7 | 17.4 |
| Melanoma* M14 | 1.0 | 0.9 | 3.5 |
| Melanoma* LOXIMVI | 2.9 | 1.5 | 3.2 |
| Melanoma* SK-MEL-5 | 0.8 | 0.8 | 0.9 |
| Squamous cell | 1.0 | 2.2 | 7.0 |
| carcinoma SCC-4 | | | |
| Testis Pool | 3.5 | 3.3 | 4.7 |
| Prostate ca.* (bone | 6.4 | 1.8 | 6.3 |
| met) PC-3 | | | |
| Prostate Pool | 2.1 | 2.0 | 3.9 |
| Placenta | 30.8 | 25.9 | 39.0 |
| Uterus Pool | 7.7 | 4.7 | 9.0 |
| Ovarian ca. OVCAR-3 | 4.4 | 6.1 | 15.7 |
| Ovarian ca. SK-OV-3 | 9.7 | 18.2 | 46.3 |
| Ovarian ca. OVCAR-4 | 3.7 | 5.4 | 7.1 |
| Ovarian ca. OVCAR-5 | 19.2 | 19.9 | 30.6 |
| Ovarian ca. IGROV-1 | 7.0 | 9.1 | 14.1 |
| Ovarian ca. OVCAR-8 | 1.8 | 1.9 | 2.7 |
| Ovary | 2.7 | 2.5 | 4.5 |
| Breast ca. MCF-7 | 64.6 | 81.8 | 100.0 |
| Breast ca. MDA-MB-231 | 3.1 | 2.1 | 9.2 |
| Breast ca. BT549 | 24.5 | 36.3 | 73.2 |
| Breast ca. T47D | 37.4 | 60.3 | 66.0 |
| Breast ca. MDA-N | 0.3 | 0.5 | 0.9 |
| Breast Pool | 33.2 | 9.8 | 24.1 |
| Trachea | 14.5 | 15.5 | 18.0 |
| Lung | 4.2 | 3.4 | 6.7 |
| Fetal Lung | 83.5 | 100.0 | 68.3 |
| Lung ca.NCI-N417 | 0.0 | 0.0 | 0.2 |
| Lung ca. LX-1 | 8.0 | 6.0 | 11.8 |
| Lung ca. NCI-H146 | 0.0 | 0.0 | 0.0 |
| Lung ca. SHP-77 | 0.0 | 0.0 | 0.1 |
| Lung ca. A549 | 35.4 | 0.0 | 36.6 |
| Lung ca. NCI-H526 | 0.0 | 0.0 | 0.0 |
| Lung ca. NCI-H23 | 10.9 | 13.0 | 13.4 |
| Lung ca. NCI-H460 | 7.4 | 5.8 | 17.6 |
| Lung ca. HOP-62 | 11.4 | 4.3 | 13.2 |
| Lung ca. NCI-H522 | 1.6 | 1.5 | 2.1 |
| Liver | 0.6 | 0.2 | 1.0 |
| Fetal Liver | 5.0 | 4.0 | 2.8 |
| Liver ca. HepG2 | 4.5 | 5.4 | 8.1 |
| Kidney Pool | 26.6 | 21.0 | 31.4 |
| Fetal Kidney | 9.0 | 10.7 | 7.7 |
| Renal ca. 786-0 | 6.0 | 7.9 | 10.9 |
| Renal ca. A498 | 1.2 | 2.3 | 5.2 |
| Renal ca.ACHN | 1.9 | 0.8 | 2.5 |
| Renal ca. UO-31 | 11.1 | 10.7 | 14.9 |
| Renal ca. TK-10 | 6.4 | 8.2 | 10.6 |
| Bladder | 32.5 | 24.1 | 31.9 |
| Gastric ca. | 26.8 | 23.5 | 36.3 |
| (liver met.) NCI-N87 | | | |
| Gastric ca. KATO III | 8.7 | 8.0 | 12.2 |
| Colon ca. SW-948 | 2.6 | 2.6 | 5.4 |
| Colon ca. SW480 | 13.5 | 12.3 | 25.0 |
| Colon ca.* (SW480 met) | 1.6 | 1.4 | 2.5 |
| SW620 | | | |
| Colon ca. HT29 | 7.2 | 5.7 | 14.3 |
| Colon ca. HCT-116 | 2.1 | 1.7 | 2.1 |
| Colon ca. CaCo-2 | 13.5 | 15.7 | 15.9 |
| Colon cancer tissue | 34.9 | 42.3 | 39.8 |
| Colon ca. SW1116 | 0.1 | 0.3 | 3.4 |
| Colon ca. Colo-205 | 2.7 | 2.6 | 8.8 |
| Colon ca. SW-48 | 3.3 | 4.7 | 5.4 |
| Colon Pool | 16.6 | 9.8 | 16.2 |
| Small Intestine | 7.3 | 5.5 | 9.3 |
| Pool | | | |
| Stomach Pool | 6.6 | 8.0 | 17.3 |
| Bone Marrow Pool | 5.2 | 3.3 | 7.0 |
| Fetal Heart | 4.5 | 4.6 | 2.9 |
| Heart Pool | 9.2 | 6.8 | 7.9 |
| Lymph Node Pool | 10.4 | 9.9 | 15.2 |
| Fetal Skeletal | 2.4 | 2.9 | 1.7 |
| Muscle | | | |
| Skeletal Muscle Pool | 7.7 | 8.5 | 9.8 |
| Spleen Pool | 16.0 | 22.8 | 45.7 |
| Thymus Pool | 7.5 | 6.9 | 15.9 |
| CNS cancer | 2.1 | 2.4 | 7.6 |
| (glio/astro) U87-MG | | | |
| CNS cancer | 5.4 | 2.7 | 7.9 |
| (glio/astro) U-118-MG | | | |
| CNS cancer | 0.7 | 1.2 | 2.6 |
| (neuro; met) SK-N-AS | | | |
| CNS cancer | 1.4 | 1.8 | 2.3 |
| (astro) SF-539 | | | |
| CNS cancer | 4.7 | 5.9 | 14.1 |
| (astro) SNB-75 | | | |
| CNS cancer | 6.2 | 10.7 | 11.1 |
| (glio) SNB-19 | | | |
| CNS cancer | 16.0 | 18.8 | 31.9 |
| (glio) SF-295 | | | |
| Brain (Amygdala) Pool | 1.6 | 0.7 | 2.7 |
| Brain (cerebellum) | 1.1 | 0.3 | 1.4 |
| Brain (fetal) | 6.0 | 4.1 | 4.9 |
| Brain (Hippocampus) | 3.6 | 1.5 | 3.7 |
| Pool | | | |
| Cerebral Cortex Pool | 2.1 | 2.0 | 3.5 |
| Brain (Substantia | 2.4 | 2.0 | 2.7 |
| nigra) Pool | | | |
| Brain (Thalamus) | 2.6 | 2.2 | 4.5 |
| Pool | | | |
| Brain (whole) | 2.7 | 2.5 | 4.5 |
| Spinal Cord Pool | 2.1 | 3.2 | 3.8 |
| Adrenal Gland | 11.7 | 3.8 | 9.5 |
| Pituitary gland | 0.7 | 0.7 | 1.4 |
| Pool | | | |
| Salivary Gland | 1.9 | 1.5 | 2.5 |
| Thyroid (female) | 3.3 | 3.6 | 7.7 |
| Pancreatic ca. | 14.9 | 21.9 | 34.4 |
| CAPAN2 | | | |
| Pancreas Pool | 15.0 | 17.8 | 19.6 |

TABLE AF
Panel 1.3D

| | Rel. Exp. (%) |
|---|---|
| | Ag3116, Run |
| Tissue Name | 167617379 |
| Liver adenocarcinoma | 24.8 |
| Pancreas | 3.4 |
| Pancreatic ca. CAPAN 2 | 12.1 |
| Adrenal gland | 2.6 |
| Thyroid | 1.3 |
| Salivary gland | 0.0 |
| Pituitary gland | 2.1 |
| Brain (fetal) | 3.1 |
| Brain (whole) | 3.1 |
| Brain (amygdala) | 1.0 |
| Brain (cerebellum) | 1.0 |
| Brain (hippocampus) | 3.0 |
| Brain (substantia nigra) | 3.7 |
| Brain (thalamus) | 1.2 |
| Cerebral Cortex | 2.5 |
| Spinal cord | 3.0 |
| glio/astro U87-MG | 1.5 |
| glio/astro U-118-MG | 2.8 |
| astrocytoma SW1783 | 2.0 |
| neuro*; met SK-N-AS | 1.5 |
| astrocytoma SF-539 | 2.4 |
| astrocytoma SNB-75 | 14.5 |
| glioma SNB-19 | 0.0 |
| glioma U251 | 0.7 |
| glioma SF-295 | 6.9 |
| Heart (fetal) | 5.8 |
| Heart | 3.2 |
| Skeletal muscle (fetal) | 4.6 |
| Skeletal muscle | 2.1 |
| Bone marrow | 4.0 |
| Thymus | 3.4 |
| Spleen | 10.6 |
| Lymph node | 10.3 |
| Colorectal | 6.4 |
| Stomach | 1.8 |
| Small intestine | 3.0 |
| Colon ca. SW480 | 6.0 |
| Colon ca.* SW620 (SW480 met) | 6.1 |
| Colon ca. HT29 | 6.6 |
| Colon ca. HCT-116 | 0.0 |
| Colon ca. CaCo-2 | 11.3 |
| Colon ca. tissue (ODO3866) | 13.1 |
| Colon ca. HCC-2998 | 17.6 |
| Gastric ca.* (liver met) NCI-N87 | 11.0 |
| Bladder | 10.2 |
| Trachea | 3.9 |
| Kidney | 5.0 |
| Kidney (fetal) | 34.2 |
| Renal ca. 786-0 | 3.7 |
| Renal ca. A498 | 3.3 |
| Renal ca. RXF 393 | 17.1 |
| Renal ca. ACHN | 1.7 |
| Renal ca. UO-31 | 0.8 |
| Renal ca. TK-10 | 4.4 |
| Liver | 2.4 |
| Liver (fetal) | 4.5 |
| Liver ca. (hepatoblast) HepG2 | 4.4 |
| Lung | 25.0 |
| Lung (fetal) | 29.7 |
| Lung ca. (small cell) LX-1 | 5.5 |
| Lung ca. (small cell) NCI-H69 | 0.0 |
| Lung ca. (s. cell var.) SHP-77 | 0.0 |
| Lung ca. (large cell) NCI-H460 | 2.3 |
| Lung ca. (non-sm. cell) A549 | 14.3 |
| Lung ca. (non-s. cell) NCI-H23 | 5.0 |
| Lung ca. (non-s. cell) HOP-62 | 5.7 |
| Lung ca. (non-s. cl) NCI-H522 | 1.2 |
| Lung ca. (squam.) SW900 | 24.1 |
| Lung ca. (squam.) NCI-H596 | 0.0 |
| Mammary gland | 7.7 |
| Breast ca.* (pl. ef) MCF-7 | 57.8 |
| Breast ca.* (pl. ef) MDA-MB-231 | 0.8 |
| Breast ca.* (pl. ef) T47D | 3.5 |
| Breast ca. BT-549 | 4.8 |
| Breast ca. MDA-N | 0.0 |
| Ovary | 6.1 |
| Ovarian ca. OVCAR-3 | 3.0 |
| Ovarian ca. OVCAR-4 | 26.1 |
| Ovarian ca. OVCAR-5 | 44.8 |
| Ovarian ca. OVCAR-8 | 1.4 |
| Ovarian ca. IGROV-1 | 6.4 |
| Ovarian ca.* (ascites) SK-OV-3 | 33.2 |
| Uterus | 4.4 |
| Placenta | 6.8 |
| Prostate | 0.0 |
| Prostate ca.* (bone met) PC-3 | 2.1 |
| Testis | 0.0 |
| Melanoma Hs688(A).T | 1.0 |
| Melanoma* (met) Hs688(B).T | 3.5 |
| Melanoma UACC-62 | 0.0 |
| Melanoma M14 | 1.1 |
| Melanoma LOX IMVI | 1.2 |
| Melanoma* (met) SK-MEL-5 | 0.0 |
| Adipose | 100.0 |

TABLE AG
Panel 2D

| | Rel. Exp. (%) |
|---|---|
| | Ag3116, Run |
| Tissue Name | 169556216 |
| Normal Colon | 58.2 |
| CC Well to Mod Diff (ODO3866) | 22.7 |
| CC Margin (ODO3866) | 14.4 |
| CC Gr.2 rectosigmoid (ODO3868) | 7.5 |
| CC Margin (ODO3868) | 3.4 |
| CC Mod Diff (ODO3920) | 7.0 |
| CC Margin (ODO3920) | 6.9 |
| CC Gr.2 ascend colon (ODO3921) | 27.7 |
| CC Margin (ODO3921) | 8.4 |
| CC from Partial Hepatectomy (ODO4309) Mets | 34.9 |
| Liver Margin (ODO4309) | 8.5 |
| Colon mets to lung (OD04451-01) | 12.2 |
| Lung Margin (OD04451-02) | 21.8 |
| Normal Prostate 6546-1 | 2.9 |
| Prostate Cancer (OD04410) | 7.4 |
| Prostate Margin (OD04410) | 8.2 |
| Prostate Cancer (OD04720-01) | 6.6 |
| Prostate Margin (OD04720-02) | 21.8 |
| Normal Lung 061010 | 42.6 |
| Lung Met to Muscle (ODO4286) | 15.0 |
| Muscle Margin (ODO4286) | 9.5 |
| Lung Malignant Cancer (OD03126) | 17.4 |
| Lung Margin (OD03126) | 59.5 |
| Lung Cancer (OD04404) | 53.6 |
| Lung Margin (OD04404) | 45.1 |
| Lung Cancer (OD04565) | 10.4 |
| Lung Margin (OD04565) | 10.8 |
| Lung Cancer (OD04237-01) | 39.8 |
| Lung Margin (OD04237-02) | 65.5 |
| Ocular Mel Met to Liver (ODO4310) | 1.6 |
| Liver Margin (ODO4310) | 9.9 |
| Melanoma Mets to Lung (OD04321) | 2.0 |
| Lung Margin (OD04321) | 50.7 |
| Normal Kidney | 13.0 |
| Kidney Ca, Nuclear grade 2 (OD04338) | 16.4 |
| Kidney Margin (OD04338) | 18.4 |
| Kidney Ca Nuclear grade 1/2 (OD04339) | 10.3 |
| Kidney Margin (OD04339) | 6.5 |
| Kidney Ca, Clear cell type (OD04340) | 28.7 |
| Kidney Margin (OD04340) | 22.7 |
| Kidney Ca, Nuclear grade 3 (OD04348) | 4.5 |
| Kidney Margin (OD04348) | 6.7 |
| Kidney Cancer (OD04622-01) | 12.2 |
| Kidney Margin (OD04622-03) | 1.8 |
| Kidney Cancer (OD04450-01) | 4.0 |
| Kidney Margin (OD04450-03) | 7.1 |
| Kidney Cancer 8120607 | 3.3 |
| Kidney Margin 8120608 | 2.0 |
| Kidney Cancer 8120613 | 3.5 |
| Kidney Margin 8120614 | 2.9 |
| Kidney Cancer 9010320 | 42.0 |
| Kidney Margin 9010321 | 7.7 |
| Normal Uterus | 7.0 |
| Uterus Cancer 064011 | 18.8 |
| Normal Thyroid | 5.8 |
| Thyroid Cancer 064010 | 6.9 |
| Thyroid Cancer A302152 | 3.0 |
| Thyroid Margin A302153 | 12.1 |
| Normal Breast | 28.9 |
| Breast Cancer (OD04566) | 6.3 |
| Breast Cancer (OD04590-01) | 44.4 |
| Breast Cancer Mets (OD04590-03) | 43.5 |
| Breast Cancer Metastasis (OD04655-05) | 6.9 |
| Breast Cancer 064006 | 12.0 |
| Breast Cancer 1024 | 12.9 |
| Breast Cancer 9100266 | 6.9 |
| Breast Margin 9100265 | 6.9 |
| Breast Cancer A209073 | 7.2 |
| Breast Margin A209073 | 4.3 |
| Normal Liver | 2.3 |
| Liver Cancer 064003 | 2.1 |
| Liver Cancer 1025 | 5.8 |
| Liver Cancer 1026 | 4.2 |
| Liver Cancer 6004-T | 6.1 |
| Liver Tissue 6004-N | 6.4 |
| Liver Cancer 6005-T | 7.4 |
| Liver Tissue 6005-N | 3.9 |
| Normal Bladder | 37.1 |
| Bladder Cancer 1023 | 6.5 |
| Bladder Cancer A302173 | 14.8 |
| Bladder Cancer (OD04718-01) | 27.9 |
| Bladder Normal Adjacent (OD04718-03) | 100.0 |
| Normal Ovary | 6.3 |
| Ovarian Cancer 064008 | 31.9 |
| Ovarian Cancer (OD04768-07) | 21.9 |
| Ovary Margin (OD04768-08) | 32.5 |
| Normal Stomach | 18.8 |
| Gastric Cancer 9060358 | 14.6 |
| Stomach Margin 9060359 | 16.2 |
| Gastric Cancer 9060395 | 33.2 |
| Stomach Margin 9060394 | 24.8 |
| Gastric Cancer 9060397 | 26.8 |
| Stomach Margin 9060396 | 7.4 |
| Gastric Cancer 064005 | 27.4 |

TABLE AH
Panel 4D

| | Rel. Exp. | Rel. Exp. |
|---|---|---|
| | (%) Ag3116, | (%) Ag3551, |
| | Run | Run |
| Tissue Name | 164526105 | 166453851 |
| Secondary Th1 act | 15.6 | 38.4 |
| Secondary Th2 act | 23.0 | 56.3 |
| Secondary Tr1 act | 23.2 | 78.5 |
| Secondary Th1 rest | 2.9 | 22.8 |
| Secondary Th2 rest | 2.5 | 4.5 |
| Secondary Tr1 rest | 2.0 | 7.0 |
| Primary Th1 act | 13.5 | 18.3 |
| Primary Th2 act | 6.6 | 15.5 |
| Primary Tr1 act | 17.7 | 33.2 |
| Primary Th1 rest | 9.2 | 32.1 |
| Primary Th2 rest | 1.2 | 2.9 |
| Primary Tr1 rest | 1.7 | 3.8 |
| CD45RA CD4 lymphocyte act | 4.9 | 6.7 |
| CD45RO CD4 lymphocyte act | 11.1 | 44.8 |
| CD8 lymphocyte act | 5.3 | 12.2 |
| Secondary CD8 lymphocyte rest | 4.9 | 16.0 |
| Secondary CD8 lymphocyte act | 7.6 | 25.5 |
| CD4 lymphocyte none | 0.8 | 1.1 |
| 2ry Th1/Th2/Tr1_anti-CD95 CH11 | 3.0 | 11.0 |
| LAK cells rest | 6.8 | 5.3 |
| LAK cells IL-2 | 6.4 | 23.2 |
| LAK cells IL-2 + IL-12 | 22.4 | 73.7 |
| LAK cells IL-2 + IFN gamma | 17.4 | 44.1 |
| LAK cells IL-2 + IL-18 | 12.2 | 25.0 |
| LAK cells PMA/ionomycin | 12.3 | 20.7 |
| NK Cells IL-2 rest | 12.9 | 23.0 |
| Two Way MLR 3 day | 12.5 | 24.0 |
| Two Way MLR 5 day | 6.0 | 17.1 |
| Two Way MLR 7 day | 3.0 | 6.3 |
| PBMC rest | 4.0 | 5.4 |
| PBMC PWM | 100.0 | 49.3 |
| PBMC PHA-L | 11.8 | 5.6 |
| Ramos (B cell) none | 0.8 | 2.0 |
| Ramos (B cell) ionomycin | 16.7 | 6.5 |
| B lymphocytes PWM | 53.2 | 25.3 |
| B lymphocytes CD40L and IL-4 | 61.1 | 81.8 |
| EOL-1 dbcAMP | 0.7 | 0.4 |
| EOL-1 dbcAMP PMA/ionomycin | 2.2 | 3.0 |
| Dendritic cells none | 4.8 | 8.7 |
| Dendritic cells LPS | 12.3 | 25.2 |
| Dendritic cells anti-CD40 | 3.2 | 6.8 |
| Monocytes rest | 5.0 | 7.3 |
| Monocytes LPS | 43.8 | 100.0 |
| Macrophages rest | 8.2 | 11.7 |
| Macrophages LPS | 26.8 | 57.4 |
| HUVEC none | 0.2 | 0.5 |
| HUVEC starved | 0.6 | 1.5 |
| HUVEC IL-1beta | 0.8 | 8.2 |
| HUVEC IFN gamma | 1.4 | 1.2 |
| HUVEC TNF alpha + IFN gamma | 3.0 | 3.1 |
| HUVEC TNF alpha + IL4 | 2.5 | 2.6 |
| HUVEC IL-11 | 0.5 | 0.5 |
| Lung Microvascular EC none | 0.0 | 0.1 |
| Lung Microvascular EC | 4.2 | 2.8 |
| TNFalpha + IL-1beta | | |
| Microvascular Dermal EC none | 0.1 | 0.1 |
| Microsvasular Dermal EC | 5.7 | 7.3 |
| TNFalpha + IL-1beta | | |
| Bronchial epithelium | 2.4 | 1.5 |
| TNFalpha + IL1beta | | |
| Small airway epithelium none | 0.6 | 1.1 |
| Small airway epithelium | 5.5 | 5.0 |
| TNFalpha + IL-1beta | | |
| Coronery artery SMC rest | 1.0 | 0.8 |
| Coronery artery SMC | 0.7 | 0.6 |
| TNFalpha + IL-1beta | | |
| Astrocytes rest | 0.5 | 1.0 |
| Astrocytes TNFalpha + IL-1beta | 14.9 | 61.1 |
| KU-812 (Basophil) rest | 0.2 | 0.2 |
| KU-812 (Basophil) PMA/ionomycin | 1.0 | 1.5 |
| CCD1106 (Keratinocytes) none | 0.4 | 0.5 |
| CCD1106 (Keratinocytes) | 0.8 | 12.4 |
| TNFalpha + IL-1beta | | |
| Liver cirrhosis | 1.1 | 5.3 |
| Lupus kidney | 1.1 | 4.8 |
| NCI-H292 none | 8.4 | 9.7 |
| NCI-H292 IL-4 | 17.6 | 18.4 |
| NCI-H292 IL-9 | 6.5 | 5.3 |
| NCI-H292 IL-13 | 9.2 | 12.0 |
| NCI-H292 IFN gamma | 4.3 | 3.5 |
| HPAEC none | 0.5 | 0.5 |
| HPAEC TNF alpha + IL-1 beta | 8.2 | 11.0 |
| Lung fibroblast none | 0.2 | 1.0 |
| Lung fibroblast | 1.7 | 9.8 |
| TNF alpha + IL-1 beta | | |
| Lung fibroblast IL-4 | 3.3 | 3.2 |
| Lung fibroblast IL-9 | 0.9 | 0.5 |
| Lung fibroblast IL-13 | 1.4 | 1.8 |
| Lung fibroblast IFN gamma | 3.4 | 4.0 |
| Dermal fibroblast CCD1070 rest | 1.9 | 1.1 |
| Dermal fibroblast CCD1070 TNF alpha | 11.9 | 13.7 |
| Dermal fibroblast CCD1070 IL-1 beta | 6.1 | 6.3 |
| Dermal fibroblast IFN gamma | 0.6 | 0.9 |
| Dermal fibroblast IL-4 | 4.2 | 6.7 |
| IBD Colitis 2 | 1.1 | 4.1 |
| IBD Crohn's | 1.8 | 6.0 |
| Colon | 2.6 | 15.7 |
| Lung | 8.2 | 7.5 |
| Thymus | 2.3 | 3.5 |
| Kidney | 4.2 | 3.8 |

TABLE AI
Panel 5D

| | Rel. Exp. | Rel. Exp. |
|---|---|---|
| | (%) Ag3116, | (%) Ag4828, |
| | Run | Run |
| Tissue Name | 170863008 | 219436967 |
| 97457_Patient-02go_adipose | 33.4 | 33.9 |
| 97476_Patient-07sk_skeletal muscle | 31.2 | 33.4 |
| 97477_Patient-07ut_uterus | 7.7 | 59.5 |
| 97478_Patient-07pl_placenta | 62.0 | 39.8 |
| 97481_Patient-08sk_skeletal muscle | 20.0 | 25.9 |
| 97482_Patient-08ut_uterus | 33.4 | 19.8 |
| 97483_Patient-08pl_placenta | 58.6 | 41.5 |
| 97486_Patient-09sk_skeletal muscle | 3.7 | 6.5 |
| 97487_Patient-09ut_uterus | 13.6 | 8.1 |
| 97488_Patient-09pl_placenta | 41.2 | 38.4 |
| 97492_Patient-10ut_uterus | 31.9 | 30.6 |
| 97493_Patient-10pl_placenta | 74.7 | 72.7 |
| 97495_Patient-11go_adipose | 67.4 | 100.0 |
| 97496_Patient-11sk_skeletal muscle | 9.0 | 5.8 |
| 97497_Patient-11ut_uterus | 35.4 | 20.6 |
| 97498_Patient-11pl_placenta | 52.1 | 50.0 |
| 97500_Patient-12go_adipose | 100.0 | 82.4 |
| 97501_Patient-12sk_skeletal muscle | 14.2 | 19.2 |
| 97502_Patient-12ut_uterus | 51.8 | 23.7 |
| 97503_Patient-12pl_placenta | 39.5 | 57.0 |
| 94721_Donor 2 U - | 2.1 | 1.6 |
| A Mesenchymal Stem Cells | | |
| 94722_Donor 2 U - | 0.0 | 3.0 |
| B Mesenchymal Stem Cells | | |
| 94723_Donor 2 U - | 1.8 | 2.1 |
| C Mesenchymal Stem Cells | | |
| 94709_Donor 2 AM - A_adipose | 5.1 | 10.8 |
| 94710_Donor 2 AM - B_adipose | 3.2 | 9.3 |
| 94711_Donor 2 AM - C_adipose | 0.0 | 3.0 |
| 94712_Donor 2 AD - A_adipose | 12.9 | 13.7 |
| 94713_Donor 2 AD - B_adipose | 12.9 | 10.0 |
| 94714_Donor 2 AD - C_adipose | 8.8 | 6.7 |
| 94742_Donor 3 U - | 1.6 | 4.7 |
| A_Mesenchymal Stem Cells | | |
| 94743_Donor 3 U - | 4.8 | 2.8 |
| B_Mesenchymal Stem Cells | | |
| 94730_Donor 3 AM - A_adipose | 6.8 | 6.3 |
| 94731_Donor 3 AM - B_adipose | 5.3 | 2.4 |
| 94732_Donor 3 AM - C_adipose | 1.9 | 2.2 |
| 94733_Donor 3 AD - A_adipose | 2.5 | 10.2 |
| 94734_Donor 3 AD - B_adipose | 2.9 | 5.5 |
| 94735_Donor 3 AD - C_adipose | 6.7 | 4.7 |
| 77138_Liver_HepG2untreated | 13.0 | 14.4 |
| 73556_Heart_Cardiac stromal | 9.1 | 1.9 |
| cells (primary) | | |
| 81735_Small Intestine | 20.0 | 17.2 |
| 72409_Kidney_Proximal | 0.0 | 0.9 |
| Convoluted Tubule | | |
| 82685_Small | 13.5 | 19.1 |
| intestine_Duodenum | | |
| 90650_Adrenal_Adrenocortical | 7.3 | 8.8 |
| adenoma | | |
| 72410_Kidney_HRCE | 9.9 | 7.6 |
| 72411_Kidney_HRE | 5.9 | 13.5 |
| 73139_Uterus_Uterine smooth | 2.5 | 2.0 |
| muscle cells | | |

TABLE AJ
general oncology screening panel_v_2.4

| | Rel. Exp. |
|---|---|
| | (%) Ag3551, |
| | Run |
| Tissue Name | 259737946 |
| Colon cancer 1 | 26.6 |
| Colon NAT 1 | 9.4 |
| Colon cancer 2 | 32.3 |
| Colon NAT 2 | 7.1 |
| Colon cancer 3 | 69.3 |
| Colon NAT 3 | 41.5 |
| Colon malignant cancer 4 | 96.6 |
| Colon NAT 4 | 5.6 |
| Lung cancer 1 | 34.6 |
| Lung NAT 1 | 5.4 |
| Lung cancer 2 | 100.0 |
| Lung NAT 2 | 15.0 |
| Squamous cell carcinoma 3 | 37.6 |
| Lung NAT 3 | 2.8 |
| Metastatic melanoma 1 | 43.8 |
| Melanoma 2 | 5.0 |
| Melanoma 3 | 2.4 |
| Metastatic melanoma 4 | 69.3 |
| Metastatic melanoma 5 | 93.3 |
| Bladder cancer 1 | 2.2 |
| Bladder NAT 1 | 0.0 |
| Bladder cancer 2 | 5.0 |
| Bladder NAT 2 | 0.0 |
| Bladder NAT 3 | 1.5 |
| Bladder NAT 4 | 5.8 |
| Prostate adenocarcinoma 1 | 29.9 |
| Prostate adenocarcinoma 2 | 1.5 |
| Prostate adenocarcinoma 3 | 2.9 |
| Prostate adenocarcinoma 4 | 69.3 |
| Prostate NAT 5 | 1.3 |
| Prostate adenocarcinoma 6 | 2.1 |
| Prostate adenocarcinoma 7 | 5.5 |
| Prostate adenocarcinoma 8 | 1.5 |
| Prostate adenocarcinoma 9 | 19.1 |
| Prostate NAT 10 | 0.0 |
| Kidney cancer 1 | 38.2 |
| Kidney NAT 1 | 13.9 |
| Kidney cancer 2 | 66.9 |
| Kidney NAT 2 | 19.3 |
| Kidney cancer 3 | 27.2 |
| Kidney NAT 3 | 12.1 |
| Kidney cancer 4 | 20.4 |
| Kidney NAT 4 | 6.3 |

CNS_neurodegeneration_v1.0 Summary: Ag3551 This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_{—v}1.4 Summary: Ag3116/Ag3551/Ag4828 Results of three experiments with two different probes and primer sets are in excellent agreement. Highest expression of this gene is detected in adipose, fetal lung, and breast cancer MCF-7 cell lines (CTs=27-30). Interestingly, this gene is expressed at much higher levels in fetal (CTs=27-30) when compared to adult lung (CT=31-35). This observation suggests that expression of this gene can be used to distinguish fetal from adult lung. In addition, the relative overexpression of this gene in fetal lung suggests that the protein product may enhance lung growth or development in the fetus and thus may also act in a regenerative capacity in the adult. Therefore, therapeutic modulation of the protein encoded by this gene could be useful in treatment of lung related diseases.

In addition significant expression of this gene is found in a number of cancer (pancreatic, CNS, colon, lung, breast, ovary, prostate, melanoma) cell lines. Therefore, therapeutic modulation of the activity of this gene or its protein product, through the use of small molecule drugs, might be beneficial in the treatment of these cancers.

Among tissues with metabolic or endocrine function, this gene is expressed at high to moderate levels in pancreas, adipose, adrenal gland, thyroid, skeletal muscle, heart, fetal liver and the gastrointestinal tract. Therefore, therapeutic modulation of the activity of this gene may prove useful in the treatment of endocrine/metabolically related diseases, such as obesity and diabetes.

This gene encodes a protein that is homologous to mitogen-activated protein kinase kinase kinase 8 (MAP3K8)(COT proto-oncogene serine/threonine-protein kinase) (C-COT) (Cancer osaka thyroid oncogene). COT is able to enhance the TNF alpha production and to activate NF-kB. Both events are connected with insulin resistance and type II diabetes (1, 2, 3). Inhibition of COT kinase would prevent overproduction of TNF alpha and activation of NF-kB, thus improving insulin resistance and diabetes.

In addition, this gene is expressed at high levels in all regions of the central nervous system examined, including amygdala, hippocampus, substantia nigra, thalamus, cerebellum, cerebral cortex, and spinal cord. Recently, MKK6, a related protein, has been shown to associated with Alzheimer's disease (4). Therefore, based on the homology of this protein to MKK6 and the presence of this gene in the brain, we predict that this putative MAP3K8 may play a role in central nervous system disorders such as Alzheimer's disease, Parkinson's disease, epilepsy, multiple sclerosis, schizophrenia and depression.

Ag3551 Results from one experiment (run 213391203) are not included. The amp plot indicates that there were experimental difficulties with this run. (Data not shown).

References:

1. Ballester A, Velasco A, Tobena R, Alemany S. Cot kinase activates tumor necrosis factor-alpha gene expression in a cyclosporin A-resistant manner. J. Biol. Chem. 1998. 273, 14099-106. PMID: 9603908.

2. Bierhaus A, Schiekofer S, Schwaninger M, Andrassy M, Humpert P M, Chen J, Hong M, Luther T, Henle T, Kloting I, Morcos M, Hofmann M, Tritschler H, Weigle B, Kasper M, Smith M, Perry G, Schmidt A M, Stem D M, Haring H U, Schleicher E, Nawroth P P. Diabetes-associated sustained activation of the transcription factor nuclear factor-kappaB. Diabetes, 2001 50, 2792-808. PMID: 11723063.

3. Belich M P, Salmeron A, Johnston L H, Ley S C. TPL-2 kinase regulates the proteolysis of the NF-kappaB-inhibitory protein NF-kappaB1 p105. Nature. 1999 397, 363-8.PMID: 9950430.

4. Zhu X, Rottkamp C A, Hartzler A, Sun Z, Takeda A, Boux H, Shimohama S, Perry G, Smith M A. (2001) Activation of MKK6, an upstream activator of p38, in Alzheimer's disease. J Neurochem 79(2):311-8

Panel 1.3D Summary: Ag3116 Highest expression of this gene is detected in adipose (32.7). Low to moderate expression of this gene is also seen in number of ovarian cancer cell lines, liver adenocarcinoma and breast cancer MCF-7 cell line. Therefore, therapeutic modulation of the activity of this gene or its protein product, through the use of small molecule drugs, might be beneficial in the treatment of these cancers.

In addition, low expression of this gene is also seen in fetal kidney and lung. Interestingly, this gene is expressed at much higher levels in fetal (CT=34.3) when compared to adult kidney (CT=37). This observation suggests that expression of this gene can be used to distinguish fetal from adult kidney. In addition, the relative overexpression of this gene in fetal lung suggests that the protein product may enhance lung growth or development in the fetus and thus may also act in a regenerative capacity in the adult. Therefore, therapeutic modulation of the protein encoded by this gene could be useful in treatment of lung related diseases.

Panel 2D Summary: Ag3116 Highest expression of this gene is detected in normal bladder (OD04718-03) sample (CT=31.4). Low to moderate expression of this gene is seen in large number of normal and cancer samples. Please see Panel 1.4 for a discussion of the potential utility of this gene.

Panel 4D Summary: Ag3116/ Ag3551 Results from two experiments with same primer and probe set are in excellent agreement. Highest expression of this gene is detected in PWM treated PBMC and LPS treated monocytes (CTs=28-29). Interestingly, expression of this gene is stimulated in activated primary Th2 and Tr1, activated secondary Th1, Th2, Tr1, PWM treated PBMC, LPS treated monocytes, TNFalpha+IL-1 beta treated astrocytes and keratinocytes. Thus, expression of this gene can be used to distinguish between these activated or treated cells from the corresponding untreated or resting cells.

In addition low expression of this gene is seen in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5D Summary: Ag3116/Ag4828 Results from two experiments with different primer and probe set are in excellent agreement. Highest expression of this gene is detected in adipose tissue (CTs=29-33). Low to moderate expression of this gene is seen in wide range of samples used in this panel including adipose, skeletal muscle, uterus, and placenta. This wide spread expression of this gene in tissues with metabolic or endocrine function, suggests that this gene plays a role in endocrine/metabolically related diseases, such as obesity and diabetes.

This gene codes for mitogen-activated protein kinase kinase kinase 8 (MAP3K8). Recently, activation of MAP kinase, ERK, a related protein, by modified LDL in vascular smooth muscle cells has been implicated in the development of atherosclerosis in diabetes (Ref. 1). Therefore, MAP3K8 may also play a role in the development of this disease and therapeutic modulation of the activity of this gene or its protein product, through the use of small molecule drugs, might be beneficial in the treatment of artherosclerosis and diabetes.

References.

1. Velarde V, Jenkins A J, Christopher J, Lyons T J, Jaffa A A. (2001) Activation of MAPK by modified low-density lipoproteins in vascular smooth muscle cells. J Appl Physiol 91(3):1412-20. PMID: 11509543.

General oncology screening panel_v_{—}2.4 Summary: Ag3551 Highest expression of this gene is detected in lung cancer (CT=32.3). Moderate to low expression of this gene is detected in metastatic melanoma, prostate, lung and kidney cancers. Interestingly, expression of this gene is higher in cancer as compared to normal tissues. Therefore, expression of this gene may be used as diagnostic marker to detect the presence of these cancers and therapeutic modulation of this gene through the use of antibodies or small molecule may be useful in the treatment of metastatic melanoma, prostate, lung and kidney cancers.

B. CG101996-02: Phosphorylase Kinase Gamma Full Length.

Expression of gene CG101996-02 was assessed using the primer-probe sets Ag3882 and Ag5945, described in Tables BA and BB. Results of the RTQ-PCR runs are shown in Tables BC, BD, BE, BF and BG.

TABLE BA
Probe Name Ag3882

| | | | Start | SEQ ID |
|---|---|---|---|---|
| Primers | Sequences | Length | Position | No |
| Forward | 5′-ctgatgctgaggatgatcatg-3′ | 21 | 828 | 462 |
| Probe | TET-5′-aactaccagttggctcgcccgagt-3′-TAMRA | 25 | 855 | 463 |
| Reverse | 5′-cttcacggtgtccgagtaatc-3′ | 21 | 885 | 464 |

TABLE BB
Probe Name Ag5945

| | | | Start | SEQ ID |
|---|---|---|---|---|
| Primers | Sequences | Length | Position | No |
| Forward | 5′-attcttgtcaagctccttcaaga-3′ | 23 | 45 | 465 |
| Probe | TET-5′-caagcacttaaccagccacccagagt-3′-TAMRA | 26 | 73 | 466 |
| Reverse | 5′-gtcatgctcagatcttcagtga-3′ | 22 | 103 | 467 |

TABLE BC
AI_comprehensive panel_v1.0

| | Rel. Exp. (%) |
|---|---|
| | Ag5945, Run |
| Tissue Name | 248201924 |
| 110967 COPD-F | 0.8 |
| 110980 COPD-F | 3.8 |
| 110968 COPD-M | 1.0 |
| 110977 COPD-M | 6.4 |
| 110989 Emphysema-F | 0.4 |
| 110992 Emphysema-F | 1.9 |
| 110993 Emphysema-F | 1.2 |
| 110994 Emphysema-F | 0.0 |
| 110995 Emphysema-F | 2.7 |
| 110996 Emphysema-F | 0.0 |
| 110997 Asthma-M | 0.0 |
| 111001 Asthma-F | 1.5 |
| 111002 Asthma-F | 1.1 |
| 111003 Atopic Asthma-F | 0.4 |
| 111004 Atopic Asthma-F | 0.4 |
| 111005 Atopic Asthma-F | 0.0 |
| 111006 Atopic Asthma-F | 0.3 |
| 111417 Allergy-M | 0.2 |
| 112347 Allergy-M | 0.3 |
| 112349 Normal Lung-F | 0.6 |
| 112357 Normal Lung-F | 1.7 |
| 112354 Normal Lung-M | 2.5 |
| 112374 Crohns-F | 0.9 |
| 112389 Match Control Crohns-F | 1.2 |
| 112375 Crohns-F | 2.8 |
| 112732 Match Control Crohns-F | 1.9 |
| 112725 Crohns-M | 0.0 |
| 112387 Match Control Crohns-M | 0.4 |
| 112378 Crohns-M | 0.1 |
| 112390 Match Control Crohns-M | 3.2 |
| 112726 Crohns-M | 0.6 |
| 112731 Match Control Crohns-M | 1.2 |
| 112380 Ulcer Col-F | 0.0 |
| 112734 Match Control Ulcer Col-F | 1.9 |
| 112384 Ulcer Col-F | 0.9 |
| 112737 Match Control Ulcer Col-F | 0.4 |
| 112386 Ulcer Col-F | 0.0 |
| 112738 Match Control Ulcer Col-F | 2.6 |
| 112381 Ulcer Col-M | 0.0 |
| 112735 Match Control Ulcer Col-M | 1.4 |
| 112382 Ulcer Col-M | 0.8 |
| 112394 Match Control Ulcer Col-M | 0.3 |
| 112383 Ulcer Col-M | 0.0 |
| 112736 Match Control Ulcer Col-M | 0.4 |
| 112423 Psoriasis-F | 0.4 |
| 112427 Match Control Psoriasis-F | 4.7 |
| 112418 Psoriasis-M | 8.1 |
| 112723 Match Control Psoriasis-M | 0.0 |
| 112419 Psoriasis-M | 1.4 |
| 112424 Match Control Psoriasis-M | 0.0 |
| 112420 Psoriasis-M | 3.4 |
| 112425 Match Control Psoriasis-M | 5.1 |
| 104689 (MF) OA Bone-Backus | 55.5 |
| 104690 (MF) Adj “Normal” Bone-Backus | 72.7 |
| 104691 (MF) OA Synovium-Backus | 41.5 |
| 104692 (BA) OA Cartilage-Backus | 30.8 |
| 104694 (BA) OA Bone-Backus | 20.3 |
| 104695 (BA) Adj “Normal” Bone-Backus | 69.3 |
| 104696 (BA) OA Synovium-Backus | 14.3 |
| 104700 (SS) OA Bone-Backus | 24.1 |
| 104701 (SS) Adj “Normal” Bone-Backus | 51.4 |
| 104702 (SS) OA Synovium-Backus | 64.2 |
| 117093 OA Cartilage Rep7 | 0.2 |
| 112672 OA Bone5 | 5.9 |
| 112673 OA Synovium5 | 3.9 |
| 112674 OA Synovial Fluid cells5 | 0.2 |
| 117100 OA Cartilage Rep14 | 0.1 |
| 112756 OA Bone9 | 0.0 |
| 112757 OA Synovium9 | 100.0 |
| 112758 OA Synovial Fluid Cells9 | 0.7 |
| 117125 RA Cartilage Rep2 | 0.7 |
| 113492 Bone2 RA | 3.2 |
| 113493 Synovium2 RA | 1.8 |
| 113494 Syn Fluid Cells RA | 1.5 |
| 113499 Cartilage4 RA | 2.8 |
| 113500 Bone4 RA | 1.1 |
| 113501 Synovium4 RA | 0.9 |
| 113502 Syn Fluid Cells4 RA | 0.6 |
| 113495 Cartilage3 RA | 2.5 |
| 113496 Bone3 RA | 2.1 |
| 113497 Synovium3 RA | 1.6 |
| 113498 Syn Fluid Cells3 RA | 2.1 |
| 117106 Normal Cartilage Rep20 | 0.0 |
| 113663 Bone3 Normal | 0.5 |
| 113664 Synovium3 Normal | 0.0 |
| 113665 Syn Fluid Cells3 Normal | 0.0 |
| 117107 Normal Cartilage Rep22 | 0.8 |
| 113667 Bone4 Normal | 0.1 |
| 113668 Synovium4 Normal | 1.5 |
| 113669 Syn Fluid Cells4 Normal | 0.8 |

TABLE BD
General_screening_panel_v1.4

| | Rel. Exp. | Rel. Exp. | Rel. Exp. |
|---|---|---|---|
| | (%) Ag3882, | (%) Ag3882, | (%) Ag3882, |
| | Run | Run | Run |
| Tissue Name | 217334262 | 222181244 | 222185729 |
| Adipose | 2.1 | 3.9 | 2.5 |
| Melanoma* Hs688(A).T | 1.1 | 1.7 | 0.9 |
| Melanoma* Hs688(B).T | 0.6 | 0.9 | 1.1 |
| Melanoma* M14 | 1.4 | 0.8 | 1.7 |
| Melanoma* LOXIMVI | 0.8 | 0.9 | 0.9 |
| Melanoma* SK-MEL-5 | 4.9 | 4.1 | 3.8 |
| Squamous cell | 1.9 | 1.5 | 1.5 |
| carcinoma SCC-4 | | | |
| Testis Pool | 0.7 | 0.7 | 0.9 |
| Prostate ca.* | 3.5 | 3.7 | 3.4 |
| (bone met) PC-3 | | | |
| Prostate Pool | 1.2 | 1.1 | 1.1 |
| Placenta | 0.6 | 0.4 | 0.8 |
| Uterus Pool | 0.1 | 0.4 | 0.3 |
| Ovarian ca. OVCAR-3 | 2.4 | 1.6 | 1.9 |
| Ovarian ca. SK-OV-3 | 1.4 | 1.3 | 2.6 |
| Ovarian ca. OVCAR-4 | 1.5 | 1.0 | 1.0 |
| Ovarian ca. OVCAR-5 | 10.0 | 6.6 | 7.9 |
| Ovarian ca. IGROV-1 | 5.0 | 4.0 | 3.5 |
| Ovarian ca. OVCAR-8 | 3.5 | 3.4 | 3.4 |
| Ovary | 1.2 | 0.6 | 1.4 |
| Breast ca. MCF-7 | 2.9 | 2.8 | 1.8 |
| Breast ca. MDA-MB-231 | 3.8 | 5.0 | 6.0 |
| Breast ca. BT 549 | 7.5 | 6.8 | 7.1 |
| Breast ca. T47D | 14.3 | 19.8 | 21.3 |
| Breast ca. MDA-N | 1.1 | 1.2 | 0.8 |
| Breast Pool | 1.6 | 2.1 | 1.6 |
| Trachea | 1.5 | 2.0 | 1.7 |
| Lung | 0.4 | 0.4 | 0.8 |
| Fetal Lung | 3.1 | 3.2 | 4.1 |
| Lung ca. NCI-N417 | 0.8 | 0.6 | 1.3 |
| Lung ca. LX-1 | 5.3 | 3.4 | 3.8 |
| Lung ca. NCI-H146 | 0.8 | 0.7 | 0.9 |
| Lung ca. SHP-77 | 12.4 | 15.2 | 13.4 |
| Lung ca. A549 | 2.9 | 3.4 | 2.5 |
| Lung ca. NCI-H526 | 1.1 | 1.1 | 0.9 |
| Lung ca. NCI-H23 | 10.2 | 9.6 | 10.4 |
| Lung ca. NCI-H460 | 2.1 | 1.6 | 0.9 |
| Lung ca. HOP-62 | 2.6 | 3.0 | 3.1 |
| Lung ca. NCI-H522 | 5.0 | 4.8 | 5.1 |
| Liver | 0.0 | 0.0 | 0.1 |
| Fetal Liver | 0.8 | 0.9 | 1.2 |
| Liver ca. HepG2 | 1.5 | 0.7 | 1.2 |
| Kidney Pool | 5.8 | 6.3 | 5.7 |
| Fetal Kidney | 1.5 | 2.1 | 1.6 |
| Renal ca. 786-0 | 1.8 | 1.8 | 1.9 |
| Renal ca. A498 | 1.2 | 0.9 | 1.0 |
| Renal ca. ACHN | 4.8 | 4.1 | 4.1 |
| Renal ca. UO-31 | 1.7 | 2.8 | 2.4 |
| Renal ca. TK-10 | 2.8 | 2.4 | 3.8 |
| Bladder | 1.2 | 2.6 | 1.7 |
| Gastric ca. | 3.8 | 3.8 | 5.1 |
| (liver met.) NCI-N87 | | | |
| Gastric ca. KATO III | 3.3 | 3.4 | 3.0 |
| Colon ca. SW-948 | 0.6 | 0.8 | 0.4 |
| Colon ca. SW480 | 3.9 | 5.1 | 4.9 |
| Colon ca.* (SW480 met) | 4.0 | 4.2 | 3.9 |
| SW620 | | | |
| Colon ca. HT29 | 1.4 | 0.8 | 1.3 |
| Colon ca. HCT-116 | 4.2 | 5.0 | 4.9 |
| Colon ca. CaCo-2 | 2.3 | 1.9 | 1.0 |
| Colon cancer tissue | 2.0 | 2.9 | 2.6 |
| Colon ca. SW1116 | 1.5 | 1.7 | 1.2 |
| Colon ca. Colo-205 | 1.7 | 0.8 | 1.5 |
| Colon ca. SW-48 | 0.8 | 0.9 | 0.5 |
| Colon Pool | 1.7 | 1.8 | 1.7 |
| Small Intestine Pool | 4.3 | 3.3 | 4.1 |
| Stomach Pool | 1.3 | 1.7 | 1.1 |
| Bone Marrow Pool | 0.8 | 0.7 | 0.7 |
| Fetal Heart | 1.8 | 1.4 | 1.4 |
| Heart Pool | 4.7 | 5.0 | 5.2 |
| Lymph Node Pool | 3.4 | 3.0 | 1.8 |
| Fetal Skeletal Muscle | 30.4 | 35.4 | 28.3 |
| Skeletal Muscle Pool | 100.0 | 100.0 | 100.0 |
| Spleen Pool | 1.1 | 1.6 | 0.8 |
| Thymus Pool | 2.3 | 3.2 | 3.5 |
| CNS cancer (glio/astro) | 3.4 | 4.7 | 4.8 |
| U87-MG | | | |
| CNS cancer | 3.7 | 3.7 | 5.3 |
| (glio/astro) U-118-MG | | | |
| CNS cancer | 3.3 | 2.4 | 2.8 |
| (neuro; met) SK-N-AS | | | |
| CNS cancer | 4.0 | 4.7 | 4.8 |
| (astro) SF-539 | | | |
| CNS cancer | 15.8 | 14.5 | 17.4 |
| (astro) SNB-75 | | | |
| CNS cancer | 3.2 | 3.5 | 3.6 |
| (glio) SNB-19 | | | |
| CNS cancer | 7.9 | 10.4 | 8.3 |
| (glio) SF-295 | | | |
| Brain (Amygdala) | 4.3 | 4.7 | 4.2 |
| Pool | | | |
| Brain (cerebellum) | 17.7 | 20.6 | 16.3 |
| Brain (fetal) | 3.9 | 3.8 | 4.0 |
| Brain (Hippocampus) | 6.1 | 5.6 | 5.9 |
| Pool | | | |
| Cerebral Cortex Pool | 5.2 | 4.8 | 4.8 |
| Brain (Substantia | 6.1 | 6.6 | 6.3 |
| nigra) Pool | | | |
| Brain (Thalamus) Pool | 6.6 | 0.0 | 6.0 |
| Brain (whole) | 5.3 | 4.5 | 3.0 |
| Spinal Cord Pool | 13.7 | 13.3 | 15.9 |
| Adrenal Gland | 4.3 | 3.6 | 3.8 |
| Pituitary gland Pool | 1.0 | 0.7 | 0.7 |
| Salivary Gland | 0.8 | 0.6 | 0.2 |
| Thyroid (female) | 0.8 | 0.4 | 0.6 |
| Pancreatic ca. CAPAN2 | 3.8 | 4.4 | 5.2 |
| Pancreas Pool | 2.8 | 3.5 | 2.0 |

TABLE BF
General_screening_panel_v1.5

| | Rel. Exp. (%) |
|---|---|
| | Ag5945, Run |
| Tissue Name | 247774858 |
| Adipose | 1.6 |
| Melanoma* Hs688(A).T | 0.3 |
| Melanoma* Hs688(B).T | 0.0 |
| Melanoma* M14 | 0.0 |
| Melanoma* LOXIMVI | 0.0 |
| Melanoma* SK-MEL-5 | 0.3 |
| Squamous cell carcinoma SCC-4 | 0.0 |
| Testis Pool | 0.1 |
| Prostate ca.* (bone met) PC-3 | 0.0 |
| Prostate Pool | 0.4 |
| Placenta | 0.0 |
| Uterus Pool | 0.1 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.5 |
| Ovarian ca. OVCAR-8 | 0.3 |
| Ovary | 0.0 |
| Breast ca. MCF-7 | 0.0 |
| Breast ca. MDA-MB-231 | 0.0 |
| Breast ca. BT 549 | 0.6 |
| Breast ca. T47D | 0.0 |
| Breast ca. MDA-N | 0.1 |
| Breast Pool | 0.2 |
| Trachea | 0.2 |
| Lung | 0.0 |
| Fetal Lung | 0.4 |
| Lung ca. NCI-N417 | 0.0 |
| Lung ca. LX-1 | 0.0 |
| Lung ca. NCI-H146 | 0.0 |
| Lung ca. SHP-77 | 0.5 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.8 |
| Fetal Kidney | 0.0 |
| Renal ca. 786-0 | 0.0 |
| Renal ca. A498 | 0.1 |
| Renal ca. ACHN | 0.0 |
| Renal ca. UO-31 | 0.0 |
| Renal ca. TK-10 | 0.0 |
| Bladder | 0.2 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.3 |
| Colon ca. SW1116 | 0.0 |
| Colon ca. Colo-205 | 0.0 |
| Colon ca. SW-48 | 0.0 |
| Colon Pool | 0.2 |
| Small Intestine Pool | 0.6 |
| Stomach Pool | 0.2 |
| Bone Marrow Pool | 0.1 |
| Fetal Heart | 0.4 |
| Heart Pool | 2.8 |
| Lymph Node Pool | 0.2 |
| Fetal Skeletal Muscle | 16.0 |
| Skeletal Muscle Pool | 100.0 |
| Spleen Pool | 0.1 |
| Thymus Pool | 0.1 |
| CNS cancer (glio/astro) U87-MG | 0.0 |
| CNS cancer (glio/astro) U-118-MG | 0.6 |
| CNS cancer (neuro; met) SK-N-AS | 0.1 |
| CNS cancer (astro) SF-539 | 0.1 |
| CNS cancer (astro) SNB-75 | 2.1 |
| CNS cancer (glio) SNB-19 | 0.7 |
| CNS cancer (glio) SF-295 | 0.0 |
| Brain (Amygdala) Pool | 2.3 |
| Brain (cerebellum) | 8.1 |
| Brain (fetal) | 0.7 |
| Brain (Hippocampus) Pool | 3.5 |
| Cerebral Cortex Pool | 2.0 |
| Brain (Substantia nigra) Pool | 2.5 |
| Brain (Thalamus) Pool | 3.0 |
| Brain (whole) | 2.0 |
| Spinal Cord Pool | 7.0 |
| Adrenal Gland | 1.0 |
| Pituitary gland Pool | 0.3 |
| Salivary Gland | 0.3 |
| Thyroid (female) | 0.0 |
| Pancreatic ca. CAPAN2 | 0.0 |
| Pancreas Pool | 0.3 |

TABLE BF
Panel 4.1D

| | Rel. Exp. (%) |
|---|---|
| | Ag5945, Run |
| Tissue Name | 248173662 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 1.3 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 2.6 |
| KU-812 (Basophil) PMA/ionomycin | 3.1 |
| CCD1106 (Keratinocytes) none | 0.0 |
| CCD1106 (Keratinocytes) TNFalpha + IL-1beta | 0.0 |
| Liver cirrhosis | 3.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 5.4 |
| Lung fibroblast TNF alpha + IL-1 beta | 0.0 |
| Lung fibroblast IL-4 | 3.0 |
| Lung fibroblast IL-9 | 2.2 |
| Lung fibroblast IL-13 | 0.0 |
| Lung fibroblast IFN gamma | 12.3 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 32.3 |
| Dermal fibroblast IL-4 | 15.8 |
| Dermal Fibroblasts rest | 100.0 |
| Neutrophils TNFa + LPS | 0.0 |
| Neutrophils rest | 0.0 |
| Colon | 0.0 |
| Lung | 6.0 |
| Thymus | 2.2 |
| Kidney | 2.5 |

TABLE BG
Panel 5D

| | Rel. Exp. (%) |
|---|---|
| | Ag3882, Run |
| Tissue Name | 170221179 |
| 97457_Patient-02go_adipose | 1.4 |
| 97476_Patient-07sk_skeletal muscle | 7.4 |
| 97477_Patient-07ut_uterus | 0.7 |
| 97478_Patient-07pl_placenta | 0.8 |
| 97481_Patient-08sk_skeletal muscle | 5.0 |
| 97482_Patient-08ut_uterus | 0.0 |
| 97483_Patient-08pl_placenta | 0.2 |
| 97486_Patient-09sk_skeletal muscle | 13.7 |
| 97487_Patient-09ut_uterus | 0.1 |
| 97488_Patient-09pl_placenta | 0.8 |
| 97492_Patient-10ut_uterus | 0.0 |
| 97493_Patient-10pl_placenta | 1.4 |
| 97495_Patient-11go_adipose | 1.1 |
| 97496_Patient-11sk_skeletal muscle | 47.3 |
| 97497_Patient-11ut_uterus | 0.3 |
| 97498_Patient-11pl_placenta | 0.6 |
| 97500_Patient-12go_adipose | 1.7 |
| 97501_Patient-12sk_skeletal muscle | 100.0 |
| 97502_Patient-12ut_uterus | 0.6 |
| 97503_Patient-12pl_placenta | 0.1 |
| 94721_Donor 2 U - A_Mesenchymal Stem Cells | 0.8 |
| 94722_Donor 2 U - B_Mesenchymal Stem Cells | 0.5 |
| 94723_Donor 2 U - C_Mesenchymal Stem Cells | 0.5 |
| 94709_Donor 2 AM - A_adipose | 0.2 |
| 94710_Donor 2 AM - B_adipose | 0.8 |
| 94711_Donor 2 AM - C_adipose | 0.5 |
| 94712_Donor 2 AD - A_adipose | 4.4 |
| 94713_Donor 2 AD - B_adipose | 7.5 |
| 94714_Donor 2 AD - C_adipose | 6.2 |
| 94742_Donor 3 U - A_Mesenchymal Stem Cells | 0.9 |
| 94743_Donor 3 U - B_Mesenchymal Stem Cells | 0.0 |
| 94730_Donor 3 AM - A_adipose | 0.3 |
| 94731_Donor 3 AM - B_adipose | 0.6 |
| 94732_Donor 3 AM - C_adipose | 0.9 |
| 94733_Donor 3 AD - A_adipose | 4.1 |
| 94734_Donor 3 AD - B_adipose | 0.2 |
| 94735_Donor 3 AD - C_adipose | 3.2 |
| 77138_Liver_HepG2untreated | 1.5 |
| 73556_Heart_Cardiac stromal cells (primary) | 0.0 |
| 81735_Small Intestine | 5.4 |
| 72409_Kidney_Proximal Convoluted Tubule | 0.0 |
| 82685_Small intestine_Duodenum | 0.6 |
| 90650_Adrenal_Adrenocortical adenoma | 0.2 |
| 72410_Kidney_HRCE | 0.5 |
| 72411_Kidney_HRE | 0.0 |
| 73139_Uterus_Uterine smooth muscle cells | 1.0 |

AI_comprehensive panel_v1.0 Summary: Ag5945 Highest expression is seen in OA synovium (CT=29). In addition, moderate levels of expression are also seen in a cluster of samples from OA bone, synovium, and cartilage. Thus, expression of this gene could be used to differentiate between OA derived samples and other samples on this panel and as a marker of OA. Furthermore, therapeutic modulation of the expression or function of this gene may be useful in the treatment of OA.

General_screening_panel_v1.4 Summary: Ag3882 Three experiments with the same probe and primer produce results that are in excellent agreement. Highest expression of this gene is seen in skeletal muscle (CTs=26-27). This gene is also expressed at moderate to low levels in pituitary, adipose, adrenal gland, pancreas, thyroid, fetal liver and adult and fetal skeletal muscle and heart. This widespread expression among these tissues suggests that this gene product may play a role in normal neuroendocrine and metabolic function and that disregulated expression of this gene may contribute to neuroendocrine disorders or metabolic diseases, such as obesity and diabetes.

This gene is widely expressed in this panel, with moderate expression seen in brain, colon, gastric, lung, breast, ovarian, and melanoma cancer cell lines. This expression profile suggests a role for this gene product in cell survival and proliferation. Modulation of this gene product may be useful in the treatment of cancer.

This gene is also expressed at moderate to low levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

General_screening_panel_v1.5 Summary: Ag3882 Highest expression of this gene is seen in skeletal muscle (CT=24). Overall, expression of this gene is in agreement with Panel 1.4. Please see that panel for discussion of utility of this gene.

Panel 4.1D Summary: Ag5945 Expression is limited to dermal fibroblasts, with highest expression in resting dermal fibroblasts (CT=32.3). Thus, expression of this gene could be used to differentiate between resting and activated dermal fibroblasts. This expression also suggests that this gene may be involved in inflammatory conditions of the skin.

Panel 5D Summary: Ag5945 Moderate levels of expression are seen in skeletal muscle, while this gene is not expressed in the liver derived samples on adult liver or liver cell line samples on Panels 1.4 and 1.5 and this panel.

C. CG102822-03: Glutamine Synthase.

Expression of gene CG102822-03 was assessed using the primer-probe sets Ag4225 and Ag5106, described in Tables CA and CB. Results of the RTQ-PCR runs are shown in Tables CC, CD, CE and CF.

TABLE CA
Probe Name Ag4225

| | | | Start | SEQ ID |
|---|---|---|---|---|
| Primers | Sequences | Length | Position | No |
| Forward | 5′-cagaacaccttccaccatga-3′ | 20 | 104 | 468 |
| Probe | TET-5′-ccacctcagcaagttcccacttaaat-3′-TAMRA | 26 | 124 | 469 |
| Reverse | 5′-tgaggcagggacatgtacac-3′ | 20 | 165 | 470 |

TABLE CB
Probe Name Ag5106

| | | | Start | SEQ ID |
|---|---|---|---|---|
| Primers | Sequences | Length | Position | No |
| Forward | 5′-aggaatcagcatgggagatc-3′ | 20 | 749 | 471 |
| Probe | TET-5′-ttgcatcgtgtgtgtgaagactttgg-3′-TAMRA | 26 | 792 | 472 |
| Reverse | 5′-ggcttaggatcaaaggttgc-3′ | 20 | 825 | 473 |

TABLE CC
CNS_neurodegeneration_v1.0

| | Rel. Exp. (%) | Rel. Exp. (%) |
|---|---|---|
| Tissue | Ag4225, Run | Ag5106, Run |
| Name | 249266000 | 249286585 |
| AD 1 Hippo | 10.3 | 9.6 |
| AD 2 Hippo | 17.4 | 17.9 |
| AD 3 Hippo | 4.0 | 3.6 |
| AD 4 Hippo | 4.6 | 4.8 |
| AD 5 Hippo | 67.8 | 58.2 |
| AD 6 Hippo | 100.0 | 100.0 |
| Control 2 | 18.0 | 19.9 |
| Hippo | | |
| Control 4 | 8.0 | 5.7 |
| Hippo | | |
| Control | 6.8 | 20.4 |
| (Path) 3 | | |
| Hippo | | |
| AD 1 | 10.9 | 12.2 |
| Temporal | | |
| Ctx | | |
| AD 2 | 27.5 | 28.7 |
| Temporal | | |
| Ctx | | |
| AD 3 | 6.3 | 6.2 |
| Temporal | | |
| Ctx | | |
| AD 4 | 19.6 | 24.5 |
| Temporal | | |
| Ctx | | |
| AD 5 Inf | 66.4 | 69.3 |
| Temporal | | |
| Ctx | | |
| AD 5 Sup | 36.3 | 33.7 |
| Temporal | | |
| Ctx | | |
| AD 6 Inf | 94.0 | 84.7 |
| Temporal | | |
| Ctx | | |
| AD 6 Sup | 87.7 | 84.7 |
| Temporal | | |
| Ctx | | |
| Control 1 | 9.1 | 11.1 |
| Temporal | | |
| Ctx | | |
| Control 2 | 30.4 | 28.5 |
| Temporal | | |
| Ctx | | |
| Control 3 | 15.1 | 21.5 |
| Temporal | | |
| Ctx | | |
| Control 3 | 11.3 | 9.9 |
| Temporal | | |
| Ctx | | |
| Control | 37.9 | 34.6 |
| (Path) 1 | | |
| Temporal | | |
| Ctx | | |
| Control | 29.7 | 28.9 |
| (Path) 2 | | |
| Temporal | | |
| Ctx | | |
| Control | 12.5 | 12.0 |
| (Path) 3 | | |
| Temporal | | |
| Ctx | | |
| Control | 22.8 | 22.2 |
| (Path) 4 | | |
| Temporal | | |
| Ctx | | |
| AD 1 | 11.0 | 14.2 |
| Occipital | | |
| Ctx | | |
| AD 2 | 0.0 | 0.0 |
| Occipital | | |
| Ctx | | |
| (Missing) | | |
| AD 3 | 9.0 | 7.4 |
| Occipital | | |
| Ctx | | |
| AD 4 | 19.9 | 22.4 |
| Occipital | | |
| Ctx | | |
| AD 5 | 22.7 | 23.7 |
| Occipital | | |
| Ctx | | |
| AD 6 | 28.1 | 33.2 |
| Occipital | | |
| Ctx | | |
| Control 1 | 4.7 | 4.5 |
| Occipital | | |
| Ctx | | |
| Control 2 | 37.1 | 34.2 |
| Occipital | | |
| Ctx | | |
| Control 3 | 16.0 | 19.1 |
| Occipital | | |
| Ctx | | |
| Control 4 | 8.0 | 10.2 |
| Occipital | | |
| Ctx | | |
| Control | 42.3 | 36.1 |
| (Path) 1 | | |
| Occipital | | |
| Ctx | | |
| Control | 8.1 | 6.6 |
| (Path) 2 | | |
| Occipital | | |
| Ctx | | |
| Control | 6.9 | 5.8 |
| (Path) 3 | | |
| Occipital | | |
| Ctx | | |
| Control | 10.2 | 7.4 |
| (Path) 4 | | |
| Occipital | | |
| Ctx | | |
| Control 1 | 9.3 | 10.4 |
| Parietal Ctx | | |
| Control 2 | 54.3 | 39.8 |
| Parietal Ctx | | |
| Control 3 | 10.9 | 18.9 |
| Parietal Ctx | | |
| Control | 48.6 | 41.2 |
| (Path) 1 | | |
| Parietal Ctx | | |
| Control | 21.6 | 21.6 |
| (Path) 2 | | |
| Parietal Ctx | | |
| Control | 10.5 | 9.3 |
| (Path) 3 | | |
| Parietal Ctx | | |
| Control | 26.2 | 23.7 |
| (Path) 4 | | |
| Parietal Ctx | | |

TABLE CD
General_screening_panel_v1.5

| | Rel. Exp. (%) |
|---|---|
| | Ag5106, Run |
| Tissue Name | 228727271 |
| Adipose | 26.6 |
| Melanoma* Hs688(A).T | 6.4 |
| Melanoma* Hs688(B).T | 5.8 |
| Melanoma* M14 | 7.5 |
| Melanoma* LOXIMVI | 0.2 |
| Melanoma* SK-MEL-5 | 6.9 |
| Squamous cell carcinoma SCC-4 | 8.8 |
| Testis Pool | 15.6 |
| Prostate ca.* (bone met) PC-3 | 8.8 |
| Prostate Pool | 7.1 |
| Placenta | 22.5 |
| Uterus Pool | 9.4 |
| Ovarian ca. OVCAR-3 | 11.3 |
| Ovarian ca. SK-OV-3 | 2.9 |
| Ovarian ca. OVCAR-4 | 7.6 |
| Ovarian ca. OVCAR-5 | 27.2 |
| Ovarian ca. IGROV-1 | 6.7 |
| Ovarian ca. OVCAR-8 | 3.1 |
| Ovary | 13.8 |
| Breast ca. MCF-7 | 4.4 |
| Breast ca. MDA-MB-231 | 8.0 |
| Breast ca. BT 549 | 6.3 |
| Breast ca. T47D | 7.7 |
| Breast ca. MDA-N | 3.3 |
| Breast Pool | 10.9 |
| Trachea | 38.2 |
| Lung | 5.1 |
| Fetal Lung | 27.2 |
| Lung ca. NCI-N417 | 6.9 |
| Lung ca. LX-1 | 3.0 |
| Lung ca. NCI-HI46 | 5.1 |
| Lung ca. SHP-77 | 5.8 |
| Lung ca. A549 | 3.3 |
| Lung ca. NCI-H526 | 18.9 |
| Lung ca. NCI-H23 | 1.1 |
| Lung ca. NCI-H460 | 3.5 |
| Lung ca. HOP-62 | 4.1 |
| Lung ca. NCI-H522 | 1.0 |
| Liver | 7.2 |
| Fetal Liver | 31.0 |
| Liver ca. HepG2 | 23.7 |
| Kidney Pool | 16.6 |
| Fetal Kidney | 4.9 |
| Renal ca. 786-0 | 0.0 |
| Renal ca. A498 | 0.0 |
| Renal ca. ACHN | 4.2 |
| Renal ca. UO-31 | 3.5 |
| Renal ca. TK-10 | 12.1 |
| Bladder | 27.0 |
| Gastric ca. (liver met.) NCI-N87 | 17.2 |
| Gastric ca. KATO III | 2.4 |
| Colon ca. SW-948 | 3.5 |
| Colon ca. SW480 | 11.3 |
| Colon ca.* (SW480 met) SW620 | 8.8 |
| Colon ca. HT29 | 8.1 |
| Colon ca. HCT-116 | 11.6 |
| Colon ca. CaCo-2 | 28.7 |
| Colon cancer tissue | 13.2 |
| Colon ca. SW1116 | 0.9 |
| Colon ca. Colo-205 | 0.3 |
| Colon ca. SW-48 | 3.0 |
| Colon Pool | 12.6 |
| Small Intestine Pool | 9.5 |
| Stomach Pool | 13.8 |
| Bone Marrow Pool | 5.3 |
| Fetal Heart | 11.0 |
| Heart Pool | 7.0 |
| Lymph Node Pool | 11.7 |
| Fetal Skeletal Muscle | 11.0 |
| Skeletal Muscle Pool | 61.1 |
| Spleen Pool | 10.8 |
| Thymus Pool | 8.7 |
| CNS cancer (glio/astro) U87-MG | 3.6 |
| CNS cancer (glio/astro) U-118-MG | 0.4 |
| CNS cancer (neuro; met) SK-N-AS | 7.1 |
| CNS cancer (astro) SF-539 | 14.4 |
| CNS cancer (astro) SNB-75 | 13.0 |
| CNS cancer (glio) SNB-19 | 6.8 |
| CNS cancer (glio) SF-295 | 5.1 |
| Brain (Amygdala) Pool | 26.8 |
| Brain (cerebellum) | 100.0 |
| Brain (fetal) | 13.2 |
| Brain (Hippocampus) Pool | 36.6 |
| Cerebral Cortex Pool | 64.2 |
| Brain (Substantia nigra) Pool | 45.7 |
| Brain (Thalamus) Pool | 55.9 |
| Brain (whole) | 55.9 |
| Spinal Cord Pool | 32.8 |
| Adrenal Gland | 11.3 |
| Pituitary gland Pool | 2.6 |
| Salivary Gland | 5.5 |
| Thyroid (female) | 12.2 |
| Pancreatic ca. CAPAN2 | 5.1 |
| Pancreas Pool | 12.8 |

TABLE CE
Panel 5 Islet

| | Rel. Exp. (%) | Rel. Exp. (%) | Rel. Exp. (%) |
|---|---|---|---|
| | Ag4225, Run | Ag4225, Run | Ag5106, Run |
| Tissue Name | 248989150 | 249252911 | 312852504 |
| 97457_Patient- | 36.3 | 48.6 | 42.0 |
| 02go_adipose | | | |
| 97476_Patient- | 16.7 | 17.4 | 0.0 |
| 07sk_skeletal | | | |
| muscle | | | |
| 97477_Patient- | 12.0 | 15.9 | 10.6 |
| 07ut_uterus | | | |
| 97478_Patient- | 15.4 | 27.4 | 23.3 |
| 07pl_placenta | | | |
| 99167_Bayer | 37.4 | 29.9 | 20.0 |
| Patient 1 | | | |
| 97482_Patient- | 9.0 | 12.7 | 7.3 |
| 08ut_uterus | | | |
| 97483_Patient- | 12.0 | 17.6 | 14.7 |
| 08pl_placenta | | | |
| 97486_Patient- | 7.6 | 9.3 | 9.4 |
| 09sk_skeletal | | | |
| muscle | | | |
| 97487_Patient- | 19.5 | 21.0 | 11.2 |
| 09ut_uterus | | | |
| 97488_Patient- | 9.6 | 22.2 | 13.8 |
| 09pl_placenta | | | |
| 97492_Patient- | 15.8 | 20.6 | 13.3 |
| 10ut_uterus | | | |
| 97493_Patient- | 43.2 | 52.5 | 38.4 |
| 10pl_placenta | | | |
| 97495_Patient- | 33.4 | 33.9 | 18.8 |
| 11go_adipose | | | |
| 97496_Patient- | 35.6 | 52.1 | 27.7 |
| 11sk_skeletal | | | |
| muscle | | | |
| 97497_Patient- | 18.9 | 22.8 | 19.9 |
| 11ut_uterus | | | |
| 97498_Patient- | 17.1 | 19.1 | 9.0 |
| 11pl_placenta | | | |
| 97500_Patient- | 100.0 | 100.0 | 73.2 |
| 12go_adipose | | | |
| 97501_Patient- | 63.7 | 74.2 | 59.5 |
| 12sk_skeletal | | | |
| muscle | | | |
| 97502_Patient- | 16.6 | 17.6 | 17.1 |
| 12ut_uterus | | | |
| 97503_Patient- | 25.2 | 35.6 | 35.8 |
| 12pl_placenta | | | |
| 94721_Donor 2 | 4.5 | 7.5 | 10.3 |
| U - | | | |
| A_Mesenchymal | | | |
| Stem Cells | | | |
| 94722_Donor 2 | 4.2 | 5.6 | 5.2 |
| U - | | | |
| B_Mesenchymal | | | |
| Stem Cells | | | |
| 94723_Donor 2 | 5.6 | 1.1 | 8.5 |
| U - | | | |
| C_Mesenchymal | | | |
| Stem Cells | | | |
| 94709_Donor 2 | 15.6 | 27.9 | 15.4 |
| AM - | | | |
| A_adipose | | | |
| 94710_Donor 2 | 10.6 | 18.9 | 15.3 |
| AM - | | | |
| B_adipose | | | |
| 94711_Donor 2 | 7.4 | 14.5 | 12.5 |
| AM - | | | |
| C_adipose | | | |
| 94712_Donor 2 | 17.1 | 22.1 | 34.9 |
| AD - | | | |
| A_adipose | | | |
| 94713_Donor 2 | 15.9 | 27.9 | 45.4 |
| AD - B_adipose | | | |
| 94714_Donor 2 | 16.0 | 25.5 | 29.5 |
| AD - C_adipose | | | |
| 94742_Donor 3 | 1.8 | 3.8 | 2.3 |
| U - | | | |
| A_Mesenchymal | | | |
| Stem Cells | | | |
| 94743_Donor 3 | 4.3 | 4.6 | 2.5 |
| U - | | | |
| B_Mesenchymal | | | |
| Stem Cells | | | |
| 94730_Donor 3 | 15.0 | 20.2 | 28.5 |
| AM - | | | |
| A_adipose | | | |
| 94731_Donor 3 | 9.9 | 13.7 | 46.0 |
| AM - | | | |
| B_adipose | | | |
| 94732_Donor 3 | 8.8 | 17.1 | 31.9 |
| AM - | | | |
| C_adipose | | | |
| 94733_Donor 3 | 6.7 | 6.7 | 14.1 |
| AD - | | | |
| A_adipose | | | |
| 94734_Donor 3 | 2.2 | 4.7 | 11.4 |
| AD - B_adipose | | | |
| 94735_Donor 3 | 4.4 | 4.6 | 3.7 |
| AD - C_adipose | | | |
| 77138_Liver_{—} | 70.2 | 98.6 | 100.0 |
| HepG2untreated | | | |
| 73556_Heart_{—} | 3.6 | 4.4 | 3.1 |
| Cardiac stromal | | | |
| cells (primary) | | | |
| 81735_Small | 21.6 | 19.9 | 16.4 |
| Intestine | | | |
| 72409_Kidney_{—} | 2.0 | 2.2 | 7.7 |
| Proximal | | | |
| Convoluted | | | |
| Tubule | | | |
| 82685_Small | 6.6 | 10.8 | 7.4 |
| intestine_Duo- | | | |
| denum | | | |
| 90650_Adrenal_{—} | 6.6 | 8.1 | 5.1 |
| Adrenocortical | | | |
| adenoma | | | |
| 72410_Kidney_{—} | 13.1 | 10.4 | 7.6 |
| HRCE | | | |
| 72411_Kidney_{—} | 7.5 | 9.1 | 5.2 |
| HRE | | | |
| 73139_Uterus_{—} | 2.7 | 4.5 | 8.2 |
| Uterine smooth | | | |
| muscle cells | | | |

TABLE CF
Panel 5D

| | Rel. Exp. (%) |
|---|---|
| | Ag4225, Run |
| Tissue Name | 181457566 |
| 97457_Patient-02go_adipose | 52.1 |
| 97476_Patient-07sk_skeletal muscle | 16.4 |
| 97477_Patient-07ut_uterus | 13.8 |
| 97478_Patient-07pl_placenta | 24.5 |
| 97481_Patient-08sk_skeletal muscle | 13.3 |
| 97482_Patient-08ut_uterus | 12.0 |
| 97483_Patient-08pl_placenta | 17.3 |
| 97486_Patient-09sk_skeletal muscle | 9.2 |
| 97487_Patient-09ut_uterus | 21.6 |
| 97488_Patient-09pl_placenta | 21.3 |
| 97492_Patient-10ut_uterus | 16.6 |
| 97493_Patient-10pl_placenta | 52.5 |
| 97495_Patient-11go_adipose | 39.5 |
| 97496_Patient-11sk_skeletal muscle | 51.4 |
| 97497_Patient-11ut_uterus | 24.8 |
| 97498_Patient-11pl_placenta | 23.2 |
| 97500_Patient-12go_adipose | 92.7 |
| 97501_Patient-12sk_skeletal muscle | 72.7 |
| 97502_Patient-12ut_uterus | 26.2 |
| 97503_Patient-12pl_placenta | 27.0 |
| 94721_Donor 2 U - A_Mesenchymal Stem Cells | 5.4 |
| 94722_Donor 2 U - B_Mesenchymal Stem Cells | 5.6 |
| 94723_Donor 2 U - C_Mesenchymal Stem Cells | 6.4 |
| 94709_Donor 2 AM - A_adipose | 24.3 |
| 94710_Donor 2 AM - B_adipose | 15.8 |
| 94711_Donor 2 AM - C_adipose | 11.7 |
| 94712_Donor 2 AD - A_adipose | 22.1 |
| 94713_Donor 2 AD - B_adipose | 25.2 |
| 94714_Donor 2 AD - C_adipose | 23.5 |
| 94742_Donor 3 U - A_Mesenchymal Stem Cells | 4.1 |
| 94743_Donor 3 U - B_Mesenchymal Stem Cells | 5.5 |
| 94730_Donor 3 AM - A_adipose | 26.1 |
| 94731_Donor 3 AM - B_adipose | 12.9 |
| 94732_Donor 3 AM - C_adipose | 13.0 |
| 94733_Donor 3 AD - A_adipose | 8.4 |
| 94734_Donor 3 AD - B_adipose | 4.9 |
| 94735_Donor 3 AD - C_adipose | 5.4 |
| 77138_Liver_HepG2untreated | 100.0 |
| 73556_Heart_Cardiac stromal cells (primary) | 3.5 |
| 81735_Small Intestine | 19.5 |
| 72409_Kidney_Proximal Convoluted Tubule | 2.3 |
| 82685_Small intestine_Duodenum | 10.0 |
| 90650_Adrenal_Adrenocortical adenoma | 6.4 |
| 72410_Kidney_HRCE | 10.3 |
| 72411_Kidney_HRE | 8.0 |
| 73139_Uterus_Uterine smooth muscle cells | 3.7 |

CNS_neurodegeneration_v1.0 Summary: Ag4225/Ag5106 Two experiments with two different probe and primer sets produce results that are in excellent agreement, with highest expression in the hippocampus of an Alzheimer's patient (CTs=23-24). This panel does not show differential expression of this gene in Alzheimer's disease. However, this profile confirms the expression of this gene at moderate levels in the brain. Please see Panel 1.5 for discussion of utility of this gene in the central nervous system.

General_screening_panel_v1.4 Summary: Ag4225 Results from one experiment with this gene are not included. The amp plot indicates that there were experimental difficulties with this run.

General_screening_panel_v1.5 Summary: Ag5106 Expression of this gene appears to have a brain-preferential distribution among normal tissues, with highest expression seen in the cerebellum (CT=22). This gene is also expressed at high levels throughout the CNS, including the hippocampus, thalamus, substantia nigra, amygdala, cerebellum and cerebral cortex. Therefore, therapeutic modulation of the expression or function of this gene may be useful in the treatment of neurological disorders, such as Alzheimer's disease, Parkinson's disease, schizophrenia, multiple sclerosis, stroke and epilepsy.

Among tissues with metabolic function, this gene is expressed at high levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

Panel 5 Islet Summary: Ag4225/Ag5106 Multiple experiments with two different probe and primer sets produce results that are in excellent agreement, with highest expression in a liver cell line and adipose from a diabetic patient (CTs=26.5). In addition, high to moderate levels of expression are seen in metabolic tissues, including placenta, adipose and skeletal muscle, in agreement with Panel 1.5. This gene encodes glutamine synthase (GS) and also appears to be slightly up-regulated in diabetic skeletal muscle (patient 12). Up-regulation of glutamine synthase, which is critical for glutamine production, has been reported in obesity and diabetes, as well as in some myopathies. Muscle catabolism leads to the release of glutamine and contributes to gluconeogenesis in the liver. Inhibition of GS may decrease glutamine production, inhibit gluconeogenesis and necessitate fatty acid oxidation for energy generation. Therefore, an antagonist of glutamine synthase may be beneficial in treatment of obesity and diabetes.

Panel 5D Summary: Ag4225 Highest expression is in a liver cell line (CT=26.6). Expression is in agreement with Panel 5I. Please see that panel for further discussion of expression and utility of this gene in obesity and diabetes.

D. CG103241-02: UDPGAL:GLCNAC B1,4 Galactosyltransferase.

Expression of gene CG103241-02 was assessed using the primer-probe set Ag7620, described in Table DA.

TABLE DA
Probe Name Ag7620

| | | | Start | SEQ ID |
|---|---|---|---|---|
| Primers | Sequences | Length | Position | No |
| Forward | 5′-ctgagtaaggctcagtttctgaga-3′ | 24 | 830 | 474 |
| Probe | TET-5′-tcaatggcttccccaatgagtactgg-3′-TAMRA | 26 | 855 | 475 |
| Reverse | 5′-aatcttggtaaaccggttgaag-3′ | 22 | 907 | 476 |

CNS_neurodegeneration_v1.0 Summary: Ag7620 Expression of this gene is low/undetectable in all samples on this panel (CTs>35). (Data not shown.

Panel 4.1D Summary: Ag7620 Expression of this gene is low/undetectable in all samples on this panel (CTs>35). (Data not shown.

E. CG106249-02: Kinesin.

Expression of gene CG106249-02 was assessed using the primer-probe set Ag7282, described in Table EA. Results of the RTQ-PCR runs are shown in Tables EB and EC.

TABLE EA
Probe Name Ag7282

| | | | Start | SEO ID |
|---|---|---|---|---|
| Primers | Sequences | Length | Position | No |
| Forward | 5′-atcccaaagaaggcccttat-3′ | 20 | 550 | 477 |
| Probe | TET-5′-cgtcaccataattctgtactaaatgtttgg-3′-TAMRA | 30 | 583 | 478 |
| Reverse | 5′-cccgcatccataagttcttc-3′ | 20 | 615 | 479 |

TABLE EB
CNS_neurodegeneration_v1.0

| | Rel. Exp. (%) |
|---|---|
| | Ag7282, Run |
| Tissue Name | 296560376 |
| AD 1 Hippo | 12.5 |
| AD 2 Hippo | 25.3 |
| AD 3 Hippo | 13.7 |
| AD 4 Hippo | 11.7 |
| AD 5 Hippo | 100.0 |
| AD 6 Hippo | 59.5 |
| Control 2 Hippo | 38.7 |
| Control 4 Hippo | 19.1 |
| Control (Path) 3 Hippo | 12.9 |
| AD 1 Temporal Ctx | 42.0 |
| AD 2 Temporal Ctx | 12.7 |
| AD 3 Temporal Ctx | 10.2 |
| AD 4 Temporal Ctx | 35.6 |
| AD 5 Inf Temporal Ctx | 94.0 |
| AD 5 Sup Temporal Ctx | 57.8 |
| AD 6 Inf Temporal Ctx | 33.2 |
| AD 6 Sup Temporal Ctx | 48.6 |
| Control 1 Temporal Ctx | 10.7 |
| Control 2 Temporal Ctx | 15.1 |
| Control 3 Temporal Ctx | 32.1 |
| Control 3 Temporal Ctx | 6.4 |
| Control (Path) 1 Temporal Ctx | 45.7 |
| Control (Path) 2 Temporal Ctx | 51.1 |
| Control (Path) 3 Temporal Ctx | 15.5 |
| Control (Path) 4 Temporal Ctx | 28.3 |
| AD 1 Occipital Ctx | 27.4 |
| AD 2 Occipital Ctx (Missing) | 0.0 |
| AD 3 Occipital Ctx | 8.5 |
| AD 4 Occipital Ctx | 11.0 |
| AD 5 Occipital Ctx | 33.2 |
| AD 6 Occipital Ctx | 15.7 |
| Control 1 Occipital Ctx | 7.7 |
| Control 2 Occipital Ctx | 48.0 |
| Control 3 Occipital Ctx | 38.7 |
| Control 4 Occipital Ctx | 10.5 |
| Control (Path) 1 Occipital Ctx | 57.8 |
| Control (Path) 2 Occipital Ctx | 13.1 |
| Control (Path) 3 Occipital Ctx | 7.0 |
| Control (Path) 4 Occipital Ctx | 19.1 |
| Control 1 Parietal Ctx | 12.7 |
| Control 2 Parietal Ctx | 53.6 |
| Control 3 Parietal Ctx | 21.0 |
| Control (Path) 1 Parietal Ctx | 61.1 |
| Control (Path) 2 Parietal Ctx | 28.7 |
| Control (Path) 3 Parietal Ctx | 9.7 |
| Control (Path) 4 Parietal Ctx | 31.9 |

TABLE EC
Panel 4.1D

| | Rel. Exp. (%) |
|---|---|
| | Ag7282, Run |
| Tissue Name | 296559398 |
| Secondary Th1 act | 33.2 |
| Secondary Th2 act | 35.8 |
| Secondary Tr1 act | 8.8 |
| Secondary Th1 rest | 2.5 |
| Secondary Th2 rest | 3.4 |
| Secondary Tr1 rest | 3.0 |
| Primary Th1 act | 0.0 |
| Primary Th2 act | 7.5 |
| Primary Tr1 act | 10.6 |
| Primary Th1 rest | 2.0 |
| Primary Th2 rest | 0.0 |
| Primary Tr1 rest | 0.0 |
| CD45RA CD4 lymphocyte act | 12.8 |
| CD45RO CD4 lymphocyte act | 46.0 |
| CD8 lymphocyte act | 12.2 |
| Secondary CD8 lymphocyte rest | 5.3 |
| Secondary CD8 lymphocyte act | 0.0 |
| CD4 lymphocyte none | 6.0 |
| 2ry Th1/Th2/Tr1_anti-CD95 CH11 | 5.0 |
| LAK cells rest | 9.5 |
| LAK cells IL-2 | 6.6 |
| LAK cells IL-2 + IL-12 | 0.0 |
| LAK cells IL-2 + IFN gamma | 6.8 |
| LAK cells IL-2 + IL-18 | 4.5 |
| LAK cells PMA/ionomycin | 3.7 |
| NK Cells IL-2 rest | 22.8 |
| Two Way MLR 3 day | 8.2 |
| Two Way MLR 5 day | 3.3 |
| Two Way MLR 7 day | 0.0 |
| PBMC rest | 2.4 |
| PBMC PWM | 2.4 |
| PBMC PHA-L | 8.1 |
| Ramos (B cell) none | 10.1 |
| Ramos (B cell) ionomycin | 13.0 |
| B lymphocytes PWM | 7.4 |
| B lymphocytes CD40L and IL-4 | 18.2 |
| EOL-1 dbcAMP | 16.4 |
| EOL-1 dbcAMP PMA/ionomycin | 4.7 |
| Dendritic cells none | 7.3 |
| Dendritic cells LPS | 3.0 |
| Dendritic cells anti-CD40 | 8.2 |
| Monocytes rest | 3.8 |
| Monocytes LPS | 11.6 |
| Macrophages rest | 12.5 |
| Macrophages LPS | 6.0 |
| HUVEC none | 6.3 |
| HUVEC starved | 18.3 |
| HUVEC IL-1beta | 12.6 |
| HUVEC IFN gamma | 20.3 |
| HUVEC TNF alpha + IFN gamma | 3.1 |
| HUVEC TNF alpha + IL4 | 0.0 |
| HUVEC IL-11 | 14.6 |
| Lung Microvascular EC none | 22.1 |
| Lung Microvascular EC TNFalpha + IL-1beta | 6.5 |
| Microvascular Dermal EC none | 3.3 |
| Microsvasular Dermal EC TNFalpha + IL-1beta | 0.0 |
| Bronchial epithelium TNFalpha + IL1beta | 18.7 |
| Small airway epithelium none | 24.8 |
| Small airway epithelium TNFalpha + IL-1beta | 49.0 |
| Coronery artery SMC rest | 9.8 |
| Coronery artery SMC TNFalpha + IL-1beta | 9.6 |
| Astrocytes rest | 0.0 |
| Astrocytes TNFalpha + IL-1beta | 3.5 |
| KU-812 (Basophil) rest | 38.7 |
| KU-812 (Basophil) PMA/ionomycin | 48.6 |
| CCD1106 (Keratinocytes) none | 39.8 |
| CCD1106 (Keratinocytes) TNFalpha + IL-1beta | 9.0 |
| Liver cirrhosis | 12.5 |
| NCI-H292 none | 12.5 |
| NCI-H292 IL-4 | 13.9 |
| NCI-H292 IL-9 | 26.6 |
| NCI-H292 IL-13 | 16.7 |
| NCI-H292 IFN gamma | 2.1 |
| HPAEC none | 5.1 |
| HPAEC TNF alpha + IL-1 beta | 13.8 |
| Lung fibroblast none | 26.8 |
| Lung fibroblast TNF alpha + IL-1 beta | 17.0 |
| Lung fibroblast IL-4 | 11.1 |
| Lung fibroblast IL-9 | 8.7 |
| Lung fibroblast IL-13 | 7.7 |
| Lung fibroblast IFN gamma | 20.6 |
| Dermal fibroblast CCD1070 rest | 6.9 |
| Dermal fibroblast CCD1070 TNF alpha | 6.3 |
| Dermal fibroblast CCD1070 IL-1 beta | 0.0 |
| Dermal fibroblast IFN gamma | 10.2 |
| Dermal fibroblast IL-4 | 26.2 |
| Dermal Fibroblasts rest | 24.5 |
| Neutrophils TNFa + LPS | 0.0 |
| Neutrophils rest | 4.6 |
| Colon | 4.8 |
| Lung | 2.5 |
| Thymus | 12.5 |
| Kidney | 100.0 |

CNS_neurodegeneration_v1.0 Summary: Ag7282 This panel confirms the expression of this gene at very low levels in the brains of an independent group of individuals. No differential expression of this gene was detected between Alzheimer's diseased postmortem brains and those of non-demented controls in this experiment. However, this panel confirms the expression of this gene at very low levels in the brains of an independent group of individuals. Therefore, therapeutic modulation of this gene product may be useful in the treatment of central nervous system disorders such as Parkinson's disease, epilepsy, multiple sclerosis, schizophrenia and depression.

Panel 4.1D Summary: Ag7282 Low levels of expression of this gene is seen mainly in kidney (CT=34.3). Therefore, expression of this gene may be used to distinguish kidney from other samples used in this panel. In addition, therapeutic targeting of the expression or function of this gene may modulate kidney function and be important in the treatment of inflammatory or autoimmune diseases that affect the kidney, including lupus and glomerulonephritis.

F. CG119418-01: Farnesyl-Diphosphate Farnesyltransferase 1.

Expression of gene CG119418-01 was assessed using the primer-probe set Ag4508, described in Table FA. Results of the RTQ-PCR runs are shown in Tables FB and FC.

TABLE FA
Probe Name Ag4508

| | | | Start | SEQ ID |
|---|---|---|---|---|
| Primers | Sequences | Length | Position | No |
| Forward | 5′-gaagaccccttagttggtgaag-3′ | 22 | 586 | 480 |
| Probe | TET-5′-caactctatgggcctgtttctgcaga-3′-TAMRA | 26 | 621 | 481 |
| Reverse | 5′-ccagatagtcacggatgatgtt-3′ | 22 | 652 | 482 |

TABLE FB
General_screening_panel_v1.4

| | Rel. Exp. (%) |
|---|---|
| | Ag4508, Run |
| Tissue Name | 213805830 |
| Adipose | 4.5 |
| Melanoma* Hs688(A).T | 9.2 |
| Melanoma* Hs688(B).T | 11.9 |
| Melanoma* M14 | 30.1 |
| Melanoma* LOXIMVI | 14.8 |
| Melanoma* SK-MEL-5 | 25.5 |
| Squamous cell carcinoma SCC-4 | 17.4 |
| Testis Pool | 10.2 |
| Prostate ca.* (bone met) PC-3 | 5.3 |
| Prostate Pool | 5.2 |
| Placenta | 5.0 |
| Uterus Pool | 2.7 |
| Ovarian ca. OVCAR-3 | 17.7 |
| Ovarian ca. SK-OV-3 | 25.9 |
| Ovarian ca. OVCAR-4 | 12.4 |
| Ovarian ca. OVCAR-5 | 22.2 |
| Ovarian ca. IGROV-1 | 19.1 |
| Ovarian ca. OVCAR-8 | 4.6 |
| Ovary | 8.0 |
| Breast ca. MCF-7 | 15.8 |
| Breast ca. MDA-MB-231 | 14.0 |
| Breast ca. BT 549 | 100.0 |
| Breast ca. T47D | 48.3 |
| Breast ca. MDA-N | 18.0 |
| Breast Pool | 5.1 |
| Trachea | 9.2 |
| Lung | 1.9 |
| Fetal Lung | 10.2 |
| Lung ca. NCI-N417 | 9.2 |
| Lung ca. LX-1 | 27.5 |
| Lung ca. NCI-H146 | 15.2 |
| Lung ca. SHP-77 | 35.4 |
| Lung ca. A549 | 20.7 |
| Lung ca. NCI-H526 | 8.4 |
| Lung ca. NCI-H23 | 8.8 |
| Lung ca. NCI-H460 | 6.0 |
| Lung ca. HOP-62 | 13.1 |
| Lung ca. NCI-H522 | 8.0 |
| Liver | 1.8 |
| Fetal Liver | 33.7 |
| Liver ca. HepG2 | 36.3 |
| Kidney Pool | 8.7 |
| Fetal Kidney | 4.6 |
| Renal ca. 786-0 | 14.6 |
| Renal ca. A498 | 2.0 |
| Renal ca. ACHN | 27.4 |
| Renal ca. UO-31 | 18.6 |
| Renal ca. TK-10 | 23.2 |
| Bladder | 8.8 |
| Gastric ca. (liver met.) NCI-N87 | 28.5 |
| Gastric ca. KATO III | 75.3 |
| Colon ca. SW-948 | 16.0 |
| Colon ca. SW480 | 18.3 |
| Colon ca.* (SW480 met) SW620 | 18.0 |
| Colon ca. HT29 | 17.2 |
| Colon ca. HCT-116 | 32.1 |
| Colon ca. CaCo-2 | 33.7 |
| Colon cancer tissue | 8.7 |
| Colon ca. SW1116 | 3.8 |
| Colon ca. Colo-205 | 13.2 |
| Colon ca. SW-48 | 11.9 |
| Colon Pool | 5.3 |
| Small Intestine Pool | 6.0 |
| Stomach Pool | 3.3 |
| Bone Marrow Pool | 2.7 |
| Fetal Heart | 2.7 |
| Heart Pool | 3.3 |
| Lymph Node Pool | 6.3 |
| Fetal Skeletal Muscle | 2.8 |
| Skeletal Muscle Pool | 6.9 |
| Spleen Pool | 3.0 |
| Thymus Pool | 4.0 |
| CNS cancer (glio/astro) U87-MG | 18.4 |
| CNS cancer (glio/astro) U-118-MG | 9.4 |
| CNS cancer (neuro; met) SK-N-AS | 18.3 |
| CNS cancer (astro) SF-539 | 55.5 |
| CNS cancer (astro) SNB-75 | 20.4 |
| CNS cancer (glio) SNB-19 | 16.5 |
| CNS cancer (glio) SF-295 | 15.9 |
| Brain (Amygdala) Pool | 7.3 |
| Brain (cerebellum) | 10.1 |
| Brain (fetal) | 22.1 |
| Brain (Hippocampus) Pool | 8.1 |
| Cerebral Cortex Pool | 8.9 |
| Brain (Substantia nigra) Pool | 7.5 |
| Brain (Thalamus) Pool | 11.3 |
| Brain (whole) | 12.9 |
| Spinal Cord Pool | 11.3 |
| Adrenal Gland | 15.5 |
| Pituitary gland Pool | 2.1 |
| Salivary Gland | 7.6 |
| Thyroid (female) | 3.9 |
| Pancreatic ca. CAPAN2 | 36.9 |
| Pancreas Pool | 5.4 |

TABLE FC
Panel 5 Islet

| | Rel. Exp. (%) |
|---|---|
| | Ag4508, Run |
| Tissue Name | 200923967 |
| 97457_Patient-02go_adipose | 7.7 |
| 97476_Patient-07sk_skeletal muscle | 7.4 |
| 97477_Patient-07ut_uterus | 4.5 |
| 97478_Patient-07pl_placenta | 12.4 |
| 99167_Bayer Patient 1 | 30.8 |
| 97482_Patient-08ut_uterus | 3.4 |
| 97483_Patient-08pl_placenta | 13.3 |
| 97486_Patient-09sk_skeletal muscle | 5.5 |
| 97487_Patient-09ut_uterus | 7.7 |
| 97488_Patient-09pl_placenta | 7.0 |
| 97492_Patient-10ut_uterus | 8.0 |
| 97493_Patient-10pl_placenta | 23.8 |
| 97495_Patient-11go_adipose | 7.1 |
| 97496_Patient-11sk_skeletal muscle | 16.5 |
| 97497_Patient-11ut_uterus | 9.6 |
| 97498_Patient-11pl_placenta | 7.5 |
| 97500_Patient-12go_adipose | 13.0 |
| 97501_Patient-12sk_skeletal muscle | 47.3 |
| 97502_Patient-12ut_uterus | 8.8 |
| 97503_Patient-12pl_placenta | 13.0 |
| 94721_Donor 2 U - A_Mesenchymal Stem Cells | 17.6 |
| 94722_Donor 2 U - B_Mesenchymal Stem Cells | 8.8 |
| 94723_Donor 2 U - C_Mesenchymal Stem Cells | 11.4 |
| 94709_Donor 2 AM - A_adipose | 9.8 |
| 94710_Donor 2 AM - B_adipose | 7.7 |
| 94711_Donor 2 AM - C_adipose | 5.5 |
| 94712_Donor 2 AD - A_adipose | 14.6 |
| 94713_Donor 2 AD - B_adipose | 18.8 |
| 94714_Donor 2 AD - C_adipose | 16.5 |
| 94742_Donor 3 U - A_Mesenchymal Stem Cells | 5.7 |
| 94743_Donor 3 U - B_Mesenchymal Stem Cells | 9.0 |
| 94730_Donor 3 AM - A_adipose | 10.1 |
| 94731_Donor 3 AM - B_adipose | 5.7 |
| 94732_Donor 3 AM - C_adipose | 7.1 |
| 94733_Donor 3 AD - A_adipose | 20.3 |
| 94734_Donor 3 AD - B_adipose | 6.7 |
| 94735_Donor 3 AD - C_adipose | 16.2 |
| 77138_Liver_HepG2untreated | 100.0 |
| 73556_Heart_Cardiac stromal cells (primary) | 11.5 |
| 81735_Small Intestine | 21.6 |
| 72409_Kidney_Proximal Convoluted Tubule | 20.9 |
| 82685_Small intestine_Duodenum | 7.0 |
| 90650_Adrenal_Adrenocortical adenoma | 5.4 |
| 72410_Kidney_HRCE | 58.6 |
| 72411_Kidney_HRE | 50.0 |
| 73139_Uterus_Uterine smooth muscle cells | 20.0 |

General_screening panel_v.1.4 Summary: Ag4508 Highest expression of this gene is detected in a breast cancer BT 549 cell line (CT=23.6). High expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at high levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Interestingly, this gene is expressed at much higher levels in fetal (CT=25) when compared to adult liver (CT=29). This observation suggests that expression of this gene can be used to distinguish fetal from adult liver. In addition, the relative overexpression of this gene in fetal tissue suggests that the protein product may enhance liver growth or development in the fetus and thus may also act in a regenerative capacity in the adult. Therefore, therapeutic modulation of the protein encoded by this gene could be useful in treatment of liver related diseases.

Panel 5 Islet Summary: Ag4508 Highest expression of this gene is detected in liver cancer HepG2 cell line (CT=25.3). This gene shows a wide spread expression in this panel, which correlates with the expression in panel 1.4. High expression of this gene is detected in islet cells, adipose, skeletal muscle, uterus, placenta, heart smooth muscle, small intestine and kidney. This gene codes for Farnesyl-diphosphate farnesyltransferase. Farnesyl-diphosphate farnesyltransferase is involoved in the cholesterol biosynthetic pathway. The operation of this pathway appears to be important for glucose homeostasis and insulin secretion in pancreatic beta cells (Flamez D, Berger V, Kruhoffer M, Orntoft T, Pipeleers D, Schuit F C., 2002, Critical role for cataplerosis via citrate in glucose-regulated insulin release. Diabetes. 2002 July;51(7):2018-24. PMID: 12086928). Therefore, therapeutic modulation of this gene product may enhance insulin secretion in Type 2 diabetes.

G. CG120359-01: Acetyl-CoA Synthetase.

Expression of gene CG120359-01 was assessed using the primer-probe set Ag4830, described in Table GA. Results of the RTQ-PCR runs are shown in Tables GB and GC.

TABLE GA
Probe Name Ag4830

| | | | Start | SEQ ID |
|---|---|---|---|---|
| Primers | Sequences | Length | Position | No |
| Forward | 5′-gtggagcattgtggacaaatac-3′ | 22 | 1182 | 483 |
| Probe | TET-5′-tgaccaagttctacacagcacccaca-3′-TAMRA | 26 | 1208 | 484 |
| Reverse | 5′-gctcatctccaaacttcatgag-3′ | 22 | 1246 | 485 |

TABLE GB
General_screening_panel_v1.4

| | Rel. Exp. (%) |
|---|---|
| | Ag4830, Run |
| Tissue Name | 213856337 |
| Adipose | 16.2 |
| Melanoma* Hs688(A).T | 13.1 |
| Melanoma* Hs688(B).T | 12.6 |
| Melanoma* M14 | 47.6 |
| Melanoma* LOXIMVI | 7.4 |
| Melanoma* SK-MEL-5 | 21.6 |
| Squamous cell carcinoma SCC-4 | 17.3 |
| Testis Pool | 9.2 |
| Prostate ca.* (bone met) PC-3 | 59.9 |
| Prostate Pool | 6.6 |
| Placenta | 16.6 |
| Uterus Pool | 5.0 |
| Ovarian ca. OVCAR-3 | 22.2 |
| Ovarian ca. SK-OV-3 | 13.8 |
| Ovarian ca. OVCAR-4 | 22.4 |
| Ovarian ca. OVCAR-5 | 45.4 |
| Ovarian ca. IGROV-1 | 56.6 |
| Ovarian ca. OVCAR-8 | 9.7 |
| Ovary | 8.5 |
| Breast ca. MCF-7 | 9.7 |
| Breast ca. MDA-MB-231 | 32.8 |
| Breast ca. BT 549 | 28.3 |
| Breast ca. T47D | 88.3 |
| Breast ca. MDA-N | 34.4 |
| Breast Pool | 9.3 |
| Trachea | 12.2 |
| Lung | 4.0 |
| Fetal Lung | 27.5 |
| Lung ca. NCI-N417 | 1.6 |
| Lung ca. LX-1 | 26.2 |
| Lung ca. NCI-H146 | 1.6 |
| Lung ca. SHP-77 | 6.8 |
| Lung ca. A549 | 13.7 |
| Lung ca. NCI-H526 | 2.1 |
| Lung ca. NCI-H23 | 19.6 |
| Lung ca. NCI-H460 | 13.3 |
| Lung ca. HOP-62 | 19.2 |
| Lung ca. NCI-H522 | 11.7 |
| Liver | 5.8 |
| Fetal Liver | 65.5 |
| Liver ca. HepG2 | 55.5 |
| Kidney Pool | 15.4 |
| Fetal Kidney | 5.7 |
| Renal ca. 786-0 | 13.6 |
| Renal ca. A498 | 8.4 |
| Renal ca. ACHN | 100.0 |
| Renal ca. UO-31 | 18.6 |
| Renal ca. TK-10 | 39.8 |
| Bladder | 20.9 |
| Gastric ca. (liver met.) NCI-N87 | 36.6 |
| Gastric ca. KATO III | 37.6 |
| Colon ca. SW-948 | 12.8 |
| Colon ca. SW480 | 88.9 |
| Colon ca.* (SW480 met) SW620 | 27.2 |
| Colon ca. HT29 | 9.9 |
| Colon ca. HCT-116 | 24.7 |
| Colon ca. CaCo-2 | 62.9 |
| Colon cancer tissue | 32.8 |
| Colon ca. SW1116 | 6.0 |
| Colon ca. Colo-205 | 7.7 |
| Colon ca. SW-48 | 48.6 |
| Colon Pool | 10.9 |
| Small Intestine Pool | 12.6 |
| Stomach Pool | 7.2 |
| Bone Marrow Pool | 4.8 |
| Fetal Heart | 11.8 |
| Heart Pool | 13.1 |
| Lymph Node Pool | 12.0 |
| Fetal Skeletal Muscle | 20.3 |
| Skeletal Muscle Pool | 44.4 |
| Spleen Pool | 5.8 |
| Thymus Pool | 10.3 |
| CNS cancer (glio/astro) U87-MG | 49.3 |
| CNS cancer (glio/astro) U-118-MG | 24.3 |
| CNS cancer (neuro; met) SK-N-AS | 24.0 |
| CNS cancer (astro) SF-539 | 14.5 |
| CNS cancer (astro)SNB-75 | 33.9 |
| CNS cancer (glio) SNB-19 | 51.4 |
| CNS cancer (glio) SF-295 | 30.8 |
| Brain (Amygdala) Pool | 9.5 |
| Brain (cerebellum) | 21.3 |
| Brain (fetal) | 11.0 |
| Brain (Hippocampus) Pool | 7.3 |
| Cerebral Cortex Pool | 10.3 |
| Brain (Substantia nigra) Pool | 12.9 |
| Brain (Thalamus) Pool | 10.8 |
| Brain (whole) | 10.6 |
| Spinal Cord Pool | 8.8 |
| Adrenal Gland | 62.4 |
| Pituitary gland Pool | 1.6 |
| Salivary Gland | 13.4 |
| Thyroid (female) | 5.8 |
| Pancreatic ca. CAPAN2 | 56.6 |
| Pancreas Pool | 11.6 |

TABLE GC
Panel 5 Islet

| | Rel. Exp. (%) |
|---|---|
| | Ag4830, Run |
| Tissue Name | 223846062 |
| 97457_Patient-02go_adipose | 27.9 |
| 97476_Patient-07sk skeletal muscle | 19.2 |
| 97477_Patient-07ut_uterus | 5.2 |
| 97478_Patient-07pl_placenta | 15.7 |
| 99167_Bayer Patient 1 | 43.8 |
| 97482_Patient-08ut_uterus | 1.1 |
| 97483_Patient-08pl_placenta | 12.5 |
| 97486_Patient-09sk skeletal muscle | 11.5 |
| 97487_Patient-09ut_uterus | 6.2 |
| 97488_Patient-09pl_placenta | 3.3 |
| 97492_Patient-10ut_uterus | 1.8 |
| 97493_Patient-10pl_placenta | 14.0 |
| 97495_Patient-11go_adipose | 14.4 |
| 97496_Patient-11sk_skeletal muscle | 5.9 |
| 97497_Patient-11ut_uterus | 1.8 |
| 97498_Patient-11pl_placenta | 6.0 |
| 97500_Patient-12go adipose | 21.9 |
| 97501_Patient-12sk_skeletal muscle | 100.0 |
| 97502_Patient-12ut_uterus | 3.3 |
| 97503_Patient-12pl_placenta | 3.2 |
| 94721_Donor 2 U - A_Mesenchymal Stem Cells | 2.5 |
| 94722_Donor 2 U - B_Mesenchymal Stem Cells | 2.4 |
| 94723_Donor 2 U - C_Mesenchymal Stem Cells | 3.4 |
| 94709_Donor 2 AM - A_adipose | 10.1 |
| 94710_Donor 2 AM - B_adipose | 11.4 |
| 94711_Donor 2 AM - C_adipose | 0.6 |
| 94712_Donor 2 AD - A_adipose | 5.3 |
| 94713_Donor 2 AD - B_adipose | 10.3 |
| 94714_Donor 2 AD - C_adipose | 10.4 |
| 94742_Donor 3 U - A_Mesenchymal Stem Cells | 1.4 |
| 94743_Donor 3 U - B_Mesenchymal Stem Cells | 13.9 |
| 94730_Donor 3 AM - A_adipose | 17.1 |
| 94731_Donor 3 AM - B_adipose | 11.7 |
| 94732_Donor 3 AM - C_adipose | 10.7 |
| 94733_Donor 3 AD - A_adipose | 85.9 |
| 94734_Donor 3 AD - B_adipose | 19.2 |
| 94735_Donor 3 AD - C_adipose | 36.1 |
| 77138_Liver_HepG2untreated | 97.3 |
| 73556_Heart_Cardiac stromal cells (primary) | 9.3 |
| 81735_Small Intestine | 78.5 |
| 72409_Kidney_Proximal Convoluted Tubule | 20.4 |
| 82685_Small intestine_Duodenum | 41.2 |
| 90650_Adrenal_Adrenocortical adenoma | 17.4 |
| 72410_Kidney_HRCE | 52.5 |
| 72411_Kidney_HRE | 25.7 |
| 73139_Uterus_Uterine smooth muscle cells | 14.4 |

General_screening_panel_v1.4 Summary: Ag4830 Highest expression of this gene is seen in a renal cancer cell line (CT=26.2). This gene is widely expressed in this panel, with high to moderate expression seen in brain, colon, gastric, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at high to moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes. This gene encodes acetyl coA synthase. Inhibiting the production of acetyl CoA from one pathway may increase the utilization (energy generation) of acetyl CoA produced from other pathways. Decreased acetyl CoA will be available for lipid synthesis. Therefore, an inhibitor of ACS may facilitate weight loss and prevent weight gain, and be useful in the treatment of obesity.

In addition, this gene is expressed at much higher levels in fetal liver tissue (CT=27) when compared to expression in the adult counterpart (CT=30). Thus, expression of this gene may be used to differentiate between the fetal and adult source of this tissue. This gene is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 5 Islet Summary: Ag4830 Highest expression of this gene is seen in diabetic skeletal muscle (CT=29) (patient 12). This gene is also expressed in other metabolic tissues, including adipose and placenta. Please see Panel 1.4 for discussion of utility of this gene in metabolic disease.

H. CG124907-01: Ornithine Decarboxylase.

Expression of gene CG124907-01 was assessed using the primer-probe set Ag4751, described in Table HA. Results of the RTQ-PCR runs are shown in Tables HB and HC.

TABLE HA
Probe Name Ag4751

| | | | Start | SEQ ID |
|---|---|---|---|---|
| Primers | Sequences | Length | Position | No |
| Forward | 5′-ctggatctgaggatgtgaaact-3′ | 22 | 894 | 486 |
| Probe | TET-5′-cgtaatcaaacccagcgttggacaaat-3′-TAMRA | 26 | 937 | 487 |
| Reverse | 5′-actccagagtctgacggaaagt-3′ | 22 | 963 | 488 |

TABLE HB
General_screening_panel_v1.4

| | Rel. Exp. (%) |
|---|---|
| | Ag4751, Run |
| Tissue Name | 219997032 |
| Adipose | 5.2 |
| Melanoma* Hs688(A).T | 6.7 |
| Melanoma* Hs688(B).T | 8.8 |
| Melanoma* M14 | 5.4 |
| Melanoma* LOXIMVI | 22.1 |
| Melanoma* SK-MEL-5 | 32.5 |
| Squamous cell carcinoma SCC-4 | 10.1 |
| Testis Pool | 6.9 |
| Prostate ca.* (bone met) PC-3 | 100.0 |
| Prostate Pool | 2.8 |
| Placenta | 0.3 |
| Uterus Pool | 1.8 |
| Ovarian ca. OVCAR-3 | 24.7 |
| Ovarian ca. SK-OV-3 | 10.0 |
| Ovarian ca. OVCAR-4 | 7.3 |
| Ovarian ca. OVCAR-5 | 9.2 |
| Ovarian ca. IGROV-1 | 18.8 |
| Ovarian ca. OVCAR-8 | 6.5 |
| Ovary | 1.5 |
| Breast ca. MCF-7 | 10.7 |
| Breast ca. MDA-MB-231 | 17.3 |
| Breast ca. BT 549 | 13.4 |
| Breast ca. T47D | 17.9 |
| Breast ca. MDA-N | 2.5 |
| Breast Pool | 4.1 |
| Trachea | 2.7 |
| Lung | 1.0 |
| Fetal Lung | 6.0 |
| Lung ca. NCI-N417 | 14.7 |
| Lung ca. LX-1 | 22.5 |
| Lung ca. NCI-H146 | 14.3 |
| Lung ca. SHP-77 | 54.0 |
| Lung ca. A549 | 13.3 |
| Lung ca. NCI-H526 | 27.9 |
| Lung ca. NCI-H23 | 29.1 |
| Lung ca. NCI-H460 | 29.1 |
| Lung ca. HOP-62 | 4.9 |
| Lung ca. NCI-H522 | 31.2 |
| Liver | 0.6 |
| Fetal Liver | 8.8 |
| Liver ca. HepG2 | 17.3 |
| Kidney Pool | 4.4 |
| Fetal Kidney | 16.6 |
| Renal ca. 786-0 | 5.8 |
| Renal ca. A498 | 1.7 |
| Renal ca. ACHN | 5.9 |
| Renal ca. UO-31 | 10.2 |
| Renal ca. TK-10 | 17.7 |
| Bladder | 8.8 |
| Gastric ca. (liver met.) NCI-N87 | 18.7 |
| Gastric ca. KATO III | 85.3 |
| Colon ca. SW-948 | 11.7 |
| Colon ca. SW480 | 49.7 |
| Colon ca * (SW480 met) SW620 | 37.4 |
| Colon ca. HT29 | 17.8 |
| Colon ca. HCT-116 | 68.3 |
| Colon ca. CaCo-2 | 27.2 |
| Colon cancer tissue | 10.3 |
| Colon ca. SW1116 | 4.7 |
| Colon ca. Colo-205 | 6.4 |
| Colon ca. SW-48 | 6.6 |
| Colon Pool | 3.7 |
| Small Intestine Pool | 2.2 |
| Stomach Pool | 2.2 |
| Bone Marrow Pool | 1.4 |
| Fetal Heart | 2.0 |
| Heart Pool | 2.1 |
| Lymph Node Pool | 2.8 |
| Fetal Skeletal Muscle | 1.8 |
| Skeletal Muscle Pool | 6.3 |
| Spleen Pool | 1.4 |
| Thymus Pool | 2.7 |
| CNS cancer (glio/astro) U87-MG | 24.0 |
| CNS cancer (glio/astro) U-118-MG | 66.4 |
| CNS cancer (neuro; met) SK-N-AS | 6.0 |
| CNS cancer (astro) SF-539 | 7.9 |
| CNS cancer (astro) SNB-75 | 8.5 |
| CNS cancer (glio) SNB-19 | 15.9 |
| CNS cancer (glio) SF-295 | 21.5 |
| Brain (Amygdala) Pool | 1.4 |
| Brain (cerebellum) | 2.3 |
| Brain (fetal) | 9.5 |
| Brain (Hippocampus) Pool | 1.8 |
| Cerebral Cortex Pool | 1.9 |
| Brain (Substantia nigra) Pool | 1.4 |
| Brain (Thalamus) Pool | 1.8 |
| Brain (whole) | 2.6 |
| Spinal Cord Pool | 1.8 |
| Adrenal Gland | 1.9 |
| Pituitary gland Pool | 1.0 |
| Salivary Gland | 1.0 |
| Thyroid (female) | 7.0 |
| Pancreatic ca. CAPAN2 | 4.2 |
| Pancreas Pool | 4.2 |

TABLE HC
Panel 5D

| | Rel. Exp. (%) |
|---|---|
| | Ag4751, Run |
| Tissue Name | 204263059 |
| 97457_Patient-02go_adipose | 9.2 |
| 97476_Patient-07sk_skeletal muscle | 7.3 |
| 97477_Patient-07ut_uterus | 11.3 |
| 97478_Patient-07pl_placenta | 1.5 |
| 97481_Patient-08sk_skeletal muscle | 8.1 |
| 97482_Patient-08ut_uterus | 10.9 |
| 97483_Patient-08pl_placenta | 0.2 |
| 97486_Patient-09sk skeletal muscle | 3.2 |
| 97487_Patient-09ut_uterus | 9.9 |
| 97488_Patient-09pl_placenta | 3.0 |
| 97492_Patient-10ut_uterus | 12.4 |
| 97493_Patient-10pl_placenta | 3.9 |
| 97495_Patient-11go_adipose | 4.0 |
| 97496_Patient-11sk_skeletal muscle | 8.0 |
| 97497_Patient-11ut_uterus | 25.2 |
| 97498_Patient-11pl_placenta | 1.2 |
| 97500_Patient-12go_adipose | 12.6 |
| 97501_Patient-12sk_skeletal muscle | 30.6 |
| 97502_Patient-12ut_uterus | 21.8 |
| 97503_Patient-12pl_placenta | 1.5 |
| 94721_Donor 2 U - A_Mesenchymal Stem Cells | 29.9 |
| 94722_Donor 2 U - B_Mesenchymal Stem Cells | 21.3 |
| 94723_Donor 2 U - C_Mesenchymal Stem Cells | 23.8 |
| 94709_Donor 2 AM - A_adipose | 29.9 |
| 94710_Donor 2 AM - B_adipose | 22.1 |
| 94711_Donor 2 AM - C_adipose | 17.3 |
| 94712_Donor 2 AD - A_adipose | 30.8 |
| 94713_Donor 2 AD - B_adipose | 41.2 |
| 94714_Donor 2 AD - C_adipose | 39.2 |
| 94742_Donor 3 U - A_Mesenchymal Stem Cells | 9.0 |
| 94743_Donor 3 U - B_Mesenchymal Stem Cells | 28.1 |
| 94730_Donor 3 AM - A_adipose | 32.1 |
| 94731_Donor 3 AM - B_adipose | 17.6 |
| 94732_Donor 3 AM - C_adipose | 17.0 |
| 94733_Donor 3 AD - A_adipose | 45.4 |
| 94734_Donor 3 AD - B_adipose | 23.8 |
| 94735_Donor 3 AD - C_adipose | 38.4 |
| 77138_Liver_HepG2untreated | 100.0 |
| 73556_Heart_Cardiac stromal cells (primary) | 11.7 |
| 81735_Small Intestine | 10.0 |
| 72409_Kidney_Proximal Convoluted Tubule | 11.8 |
| 82685_Small intestine_Duodenum | 6.5 |
| 90650_Adrenal_Adrenocortical_adenoma | 1.5 |
| 72410_Kidney_HRCE | 42.6 |
| 72411_Kidney_HRE | 41.5 |
| 73139_Uterus_Uterine smooth muscle cells | 19.2 |

General_screening_panelᵥ1.4 Summary: Ag4751 Highest expression of this gene is detected in prostate cancer PC3 cell line (CT=23.5). High expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

This gene codes for ornithine Decarboxylase 1 (ODC). ODC is one of the key enzymes in polyamine biosynthesis. Preventing the accumulation of polyamines and their antilipolytic effects by inhibition of ODC at an earlier stage of obesity may inhibit progression of the obesity. In multiple GeneCalling studies at Curagen, enzyme spermidine/spermine acetyl transferase is found to be dysregulated in various disease models. This enzyme is one of the rate-limiting enzymes in the production of polyamines, spermidine and spermine. Previously, it was shown that oxidation of polyamines leads to generation of hydrogen peroxide, which has been shown to have antilipolytic effects on adipose and may be involved in the progression of obesity.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Interestingly, this gene is expressed at much higher levels in fetal (CT=27) when compared to adult liver (CT=31). This observation suggests that expression of this gene can be used to distinguish fetal from adult liver. In addition, the relative overexpression of this gene in fetal tissue suggests that the protein product may enhance liver growth or development in the fetus and thus may also act in a regenerative capacity in the adult. Therefore, therapeutic modulation of the protein encoded by this gene could be useful in treatment of liver related diseases.

References:

1: Taylor J L, Turo K A, McCann P P, Grossberg S E. Inhibition of the differentiation of 3T3-L1 cells by interferon-beta and difluoromethyl ornithine. J. Biol. Regul. Homeost. Agents 1988 January-March;2(1):19-24. PMID: 3140600.

2: Brown A P, Morrissey R L, Crowell J A, Levine B S. Difluoromethylornithine in combination with tamoxifen in female rats: 13-week oral toxicity study. Cancer Chemother Pharmacol 1999;44(6):475-83. PMID: 10550568. 3: Olefsky J M. Comparison of the effects of insulin and insulin-like agents on different aspects of adipocyte metabolism. Horm. Metab. Res. 1979 March;11(3):209-13. PMID: 447201.

4: Richelsen B, Pedersen S B, Hougaard D M. Characterization of antilipolytic action of polyamines in isolated rat adipocytes. Biochem. J. 1989 July 15;261(2):661-5. PMID: 2476118.

5: Livingston J N, Gurny P A, Lockwood D H. Insulin-like effects of polyamines in fat cells. Mediation by H2O2 formation. J. Biol. Chem. 1977 January 25;252(2):560-2. PMID:833144.

Panel 5D Summary: Ag4751 Highest expression of this gene is detected in liver cancer HepG2 cell line (CT=29.5). This gene shows a wide spread expression in this panel, which correlates with the expression in panel 1.4. Moderate expression of this gene is detected in adipose, skeletal muscle, uterus, placenta, heart smooth muscle, small intestine and kidney. Therefore, therapeutic modulation of this gene may be useful in the treatment of obesity and diabetes including type II diabetes.

I. CG128347-02: Kinesin-Like.

Expression of gene CG128347-02 was assessed using the primer-probe set Ag5691, described in Table IA. Results of the RTQ-PCR runs are shown in Table IB.

TABLE 1A
Probe Name Ag5691

| | | | Start | SEQ ID |
|---|---|---|---|---|
| Primers | Sequences | Length | Position | No |
| Forward | 5′-gaattagacctctgctttgcaa-3′ | 22 | 164 | 489 |
| Probe | TET-5′-cacacaaacttgatgattatgaagagcttc-3′-TAMRA | 30 | 187 | 490 |
| Reverse | 5′-gctggctgtttggaataactct-3′ | 22 | 217 | 491 |

TABLE IB
Panel 4.1D

| | Rel. Exp. (%) |
|---|---|
| | Ag5691, Run |
| Tissue Name | 246504797 |
| Secondary Th1 act | 9.8 |
| Secondary Th2 act | 23.0 |
| Secondary Tr1 act | 5.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 11.9 |
| Primary Tr1 act | 10.2 |
| Primary Th1 rest | 0.0 |
| Primary Th2 rest | 2.3 |
| Primary Tr1 rest | 0.0 |
| CD45RA CD4 lymphocyte act | 8.4 |
| CD45RO CD4 lymphocyte act | 13.8 |
| CD8 lymphocyte act | 0.0 |
| Secondary CD8 lymphocyte rest | 9.2 |
| Secondary CD8 lymphocyte act | 0.6 |
| CD4 lymphocyte none | 0.9 |
| 2ry Th1/Th2/Tr1_anti-CD95 CH11 | 0.0 |
| LAK cells rest | 5.9 |
| LAK cells IL-2 | 3.3 |
| LAK cells IL-2 + IL-12 | 1.4 |
| LAK cells IL-2 + IFN gamma | 2.5 |
| LAK cells IL-2 + IL-18 | 1.5 |
| LAK cells PMA/ionomycin | 3.4 |
| NK Cells IL-2 rest | 1.5 |
| Two Way MLR 3 day | 4.8 |
| Two Way MLR 5 day | 0.0 |
| Two Way MLR 7 day | 1.6 |
| PBMC rest | 0.3 |
| PBMC PWM | 0.8 |
| PBMC PHA-L | 2.2 |
| Ramos (B cell) none | 2.2 |
| Ramos (B cell) ionomycin | 18.6 |
| B lymphocytes PWM | 10.5 |
| B lymphocytes CD40L and IL-4 | 15.1 |
| EOL-1 dbcAMP | 2.8 |
| EOL-1 dbcAMP PMA/ionomycin | 0.0 |
| Dendritic cells none | 3.2 |
| Dendritic cells LPS | 1.1 |
| Dendritic cells anti-CD40 | 0.0 |
| Monocytes rest | 0.5 |
| Monocytes LPS | 18.6 |
| Macrophages rest | 3.3 |
| Macrophages LPS | 0.0 |
| HUVEC none | 5.2 |
| HUVEC starved | 2.4 |
| HUVEC IL-1beta | 8.2 |
| HUVEC IFN gamma | 9.7 |
| HUVEC TNF alpha + IFN gamma | 0.0 |
| HUVEC TNF alpha + IL4 | 2.8 |
| HUVEC IL-11 | 6.4 |
| Lung Microvascular EC none | 20.7 |
| Lung Microvascular EC TNFalpha + IL-1beta | 3.0 |
| Microvascular Dermal EC none | 1.7 |
| Microsvascular Dermal EC TNFalpha + IL-1beta | 3.4 |
| Bronchial epithelium TNFalpha + IL1beta | 11.0 |
| Small airway epithelium none | 6.1 |
| Small airway epithelium TNFalpha + IL-1beta | 9.6 |
| Coronery artery SMC rest | 3.6 |
| Coronery artery SMC TNFalpha + IL-1beta | 7.9 |
| Astrocytes rest | 1.1 |
| Astrocytes TNFalpha + IL-1beta | 0.0 |
| KU-812 (Basophil) rest | 13.7 |
| KU-812 (Basophil) PMA/ionomycin | 11.5 |
| CCD1106 (Keratinocytes) none | 25.0 |
| CCD1106 (Keratinocytes) TNFalpha + IL-1beta | 12.6 |
| Liver cirrhosis | 9.6 |
| NCI-H292 none | 15.5 |
| NCI-H292 IL-4 | 17.8 |
| NCI-H292 IL-9 | 39.0 |
| NCI-H292 IL-13 | 28.3 |
| NCI-H292 IFN gamma | 2.8 |
| HPAEC none | 3.8 |
| HPAEC TNF alpha + IL-1 beta | 18.7 |
| Lung fibroblast none | 7.6 |
| Lung fibroblast TNF alpha + IL-1 beta | 9.0 |
| Lung fibroblast IL-4 | 12.5 |
| Lung fibroblast IL-9 | 6.8 |
| Lung fibroblast IL-13 | 1.6 |
| Lung fibroblast IFN gamma | 5.9 |
| Dermal fibroblast CCD1070 rest | 10.1 |
| Dermal fibroblast CCD1070 TNF alpha | 0.0 |
| Dermal fibroblast CCD1070 IL-1 beta | 5.4 |
| Dermal fibroblast IFN gamma | 3.3 |
| Dermal fibroblast IL-4 | 14.2 |
| Dermal Fibroblasts rest | 6.6 |
| Neutrophils TNFa + LPS | 6.7 |
| Neutrophils rest | 100.0 |
| Colon | 1.1 |
| Lung | 0.4 |
| Thymus | 10.0 |
| Kidney | 28.3 |

CNS_neurodegeneration_v1.0 Summary: Ag5691 Results from one experiment with this gene are not included. The amp plot indicates that there were experimental difficulties with this run (Data not shown).

General_screening panel_v1.5 Summary: Ag5691 Results from one experiment with this gene are not included. The amp plot indicates that there were experimental difficulties with this run (Data not shown).

Panel 4.1D Summary: AG5691 Highest expression of this gene is seen in resting neutrophils (CT=31.3). This expression is reduced to background level (CT=35.2) in neutrophils activated by TNF-alpha+LPS. This expression profile suggests that the protein encoded by this gene is produced by resting neutrophils but not by activated neutrophils. Therefore, the gene product may reduce activation of these inflammatory cells and modulation of its expression or activity may reduce or eliminate the symptoms in patients with Crohn's disease, ulcerative colitis, multiple sclerosis, chronic obstructive pulmonary disease, asthma, emphysema, rheumatoid arthritis, lupus erythematosus, or psoriasis. In addition, antagonists of this gene product may be effective in increasing the immune response in patients with AIDS or other immunodeficiencies.

J. CG135823-01 and CG135823-02: TAT.

Expression of gene CG135823-01 and CG135823-02 was assessed using the primer-probe sets Ag3173 and Ag4906, described in Tables JA and JB. Results of the RTQ-PCR runs are shown in Tables JC and JD. Please note that probe-primer set Ag4906 is specific for CG135823-01 variant.

TABLE JA
Probe Name Ag3173

| | | | Start | SEQ ID |
|---|---|---|---|---|
| Primers | Sequences | Length | Position | No |
| Forward | 5′-ctctggctgagtctatgggaat-3′ | 22 | 617 | 492 |
| Probe | TET-5′-tgaggtcaaactctacaatttgttgcca-3′-TAMRA | 28 | 639 | 493 |
| Reverse | 5′-tcaggtcaatttcccaagattt-3′ | 22 | 670 | 494 |

TABLE JB
Probe Name Ag4906

| | | | Start | SEQ ID |
|---|---|---|---|---|
| Primers | Sequences | Length | Position | No |
| Forward | 5′-ctcaggatgagggaaaagaaaa-3′ | 22 | 1796 | 495 |
| Probe | TET-5′-ccccaaccatttcctcagactcta-3′-TAMRA | 24 | 1837 | 496 |
| Reverse | 5′-tggagagagcgtgttctttct-3′ | 21 | 1861 | 497 |

TABLE JC
General_screening_panel_v1.5

| | Rel. Exp. (%) |
|---|---|
| | Ag4906, Run |
| Tissue Name | 228783186 |
| Adipose | 0.1 |
| Melanoma* Hs688 (A).T | 0.1 |
| Melanoma* Hs688 (B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.5 |
| Prostate ca.* (bone met) PC-3 | 0.0 |
| Prostate Pool | 0.0 |
| Placenta | 0.0 |
| Uterus Pool | 0.1 |
| Ovarian ca. OVCAR-3 | 0.1 |
| Ovarian ca. SK-OV-3 | 0.0 |
| Ovarian ca. OVCAR-4 | 0.0 |
| Ovarian ca. OVCAR-5 | 0.3 |
| Ovarian ca. IGROV-1 | 0.0 |
| Ovarian ca. OVCAR-8 | 0.0 |
| Ovary | 0.1 |
| Breast ca. MCF-7 | 0.1 |
| Breast ca. MDA-MB-231 | 0.0 |
| Breast ca. BT 549 | 0.0 |
| Breast ca. T47D | 0.1 |
| Breast ca. MDA-N | 0.0 |
| Breast Pool | 0.0 |
| Trachea | 0.1 |
| Lung | 0.0 |
| Fetal Lung | 0.1 |
| Lung ca. NCI-N417 | 0.0 |
| Lung ca. LX-1 | 0.1 |
| Lung ca. NCI-H146 | 0.0 |
| Lung ca. SHP-77 | 0.1 |
| Lung ca. A549 | 0.5 |
| Lung ca. NCI-H526 | 0.0 |
| Lung ca. NCI-H23 | 0.1 |
| Lung ca. NCI-H460 | 0.9 |
| Lung ca. HOP-62 | 0.0 |
| Lung ca. NCI-H522 | 0.1 |
| Liver | 100.0 |
| Fetal Liver | 8.2 |
| Liver ca. HepG2 | 7.6 |
| Kidney Pool | 0.0 |
| Fetal Kidney | 0.1 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 3.4 |
| Bladder | 0.3 |
| Gastric ca. (liver met.) NCI-N87 | 0.1 |
| Gastric ca. KATO III | 0.0 |
| Colon ca. SW-948 | 0.0 |
| Colon ca. SW480 | 0.1 |
| Colon ca.* (SW480 met) SW620 | 0.0 |
| Colon ca. HT29 | 0.0 |
| Colon ca. HCT-116 | 0.1 |
| Colon ca. CaCo-2 | 0.1 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.1 |
| Small Intestine Pool | 0.0 |
| Stomach Pool | 0.2 |
| Bone Marrow Pool | 0.0 |
| Fetal Heart | 0.0 |
| Heart Pool | 0.0 |
| Lymph Node Pool | 0.1 |
| Fetal Skeletal Muscle | 0.0 |
| Skeletal Muscle Pool | 0.0 |
| Spleen Pool | 0.0 |
| Thymus Pool | 0.1 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro)SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 0.0 |
| Brain (Hippocampus) Pool | 0.1 |
| Cerebral Cortex Pool | 0.0 |
| Brain (Substantia nigra) Pool | 0.0 |
| Brain (Thalamus) Pool | 0.0 |
| Brain (whole) | 1.0 |
| Spinal Cord Pool | 0.0 |
| Adrenal Gland | 0.3 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 0.1 |

TABLE JD
Panel 5 Islet

| | Rel. Exp. (%) |
|---|---|
| | Ag4906, Run |
| Tissue Name | 223846056 |
| 97457_Patient-02go_adipose | 0.0 |
| 97476_Patient-07sk_skeletal muscle | 0.0 |
| 97477_Patient-07ut_uterus | 0.0 |
| 97478_Patient-07pl_placenta | 0.0 |
| 99167_Bayer Patient 1 | 0.0 |
| 97482_Patient-08ut_uterus | 0.0 |
| 97483_Patient-08pl_placenta | 0.0 |
| 97486_Patient-09sk_skeletal muscle | 0.0 |
| 97487_Patient-09ut_uterus | 0.0 |
| 97488_Patient-09pl_placenta | 0.0 |
| 97492_Patient-10ut_uterus | 0.6 |
| 97493_Patient-10pl_placenta | 0.0 |
| 97495_Patient-11go_adipose | 0.0 |
| 97496_Patient-11sk_skeletal muscle | 0.0 |
| 97497_Patient-11ut_uterus | 0.0 |
| 97498_Patient-11pl_placenta | 0.0 |
| 97500_Patient-12go_adipose | 0.0 |
| 97501_Patient-12sk_skeletal muscle | 0.0 |
| 97502_Patient-12ut_uterus | 0.6 |
| 97503_Patient-12pl_placenta | 0.0 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.2 |
| 94710_Donor 2 AM - B_adipose | 0.0 |
| 94711_Donor 2 AM - C_adipose | 0.0 |
| 94712_Donor 2 AD - A_adipose | 0.4 |
| 94713_Donor 2 AD - B_adipose | 0.6 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.6 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 100.0 |
| 73556_Heart_Cardiac stromal cells (primary) | 0.0 |
| 81735_Small Intestine | 1.0 |
| 72409_Kidney_Proximal Convoluted Tubule | 0.0 |
| 82685_Small intestine_Duodenum | 0.7 |
| 90650_Adrenal_Adrenocortical adenoma | 3.1 |
| 72410_Kidney_HRCE | 0.0 |
| 72411_Kidney_HRE | 0.0 |
| 73139_Uterus_Uterine smooth muscle cells | 0.0 |

General_screening_panel_v1.5 Summary: Ag4906 This gene seems to be almost exclusively expressed in liver (CT=24.6). A lower level of expression has been detected in fetal liver (CT=28) and brain. Thus, expression of this gene could be used to differentiate between liver and fetal liver tissues. In addition, the relative overexpression of this gene in fetal liver suggests that the protein product may enhance liver growth or development in the fetus and thus may also act in a regenerative capacity in the adult. Therefore, therapeutic modulation of the protein encoded by this gene could be useful in treatment of liver and metabolic related diseases, including obesity and diabetes.

Panel 5 Islet Summary: Ag4906 This gene is expressed in hepatocyte-derived HepG2 cell line (CT=29.8), which is in accordance with the liver expression seen in panel 1.5.

K. CG140122-01: Polyamine Oxidase.

Expression of gene CG140122-01 was assessed using the primer-probe sets Ag4986 and Ag5105, described in Tables KA and KB. Results of the RTQ-PCR runs are shown in Tables KC and KD.

TABLE KA
Probe Name Ag4986

| | | | Start | SEQ ID |
|---|---|---|---|---|
| Primers | Sequences | Length | Position | No |
| Forward | 5′-gtgcagagtgtgaaacttgga-3′ | 21 | 259 | 498 |
| Probe | TET-5′-catggctcccatgggaaccctat-3′-TAMRA | 23 | 313 | 499 |
| Reverse | 5′-cgttggcttctgctagatgata-3′ | 22 | 337 | 500 |

TABLE KB
Probe Name Ag5105

| | | | Start | SEQ ID |
|---|---|---|---|---|
| Primers | Sequences | Length | Position | No |
| Forward | 5′-gaccgtgtcgctaggt-3′ | 16 | 1059 | 501 |
| Probe | TET-5′-cagtacaccagtttcttccggcca-3′-TAMRA | 24 | 1087 | 502 |
| Reverse | 5′-accttctctgttgggcag-3′ | 17 | 1114 | 503 |

TABLE KC
CNS_neurodegeneration_v1.0

| | Rel. Exp. (%) |
|---|---|
| | Ag5105, Run |
| Tissue Name | 249286379 |
| AD 1 Hippo | 27.5 |
| AD 2 Hippo | 50.7 |
| AD 3 Hippo | 18.9 |
| AD 4 Hippo | 17.1 |
| AD 5 hippo | 63.7 |
| AD 6 Hippo | 100.0 |
| Control 2 Hippo | 35.4 |
| Control 4 Hippo | 24.3 |
| Control (Path) 3 Hippo | 10.6 |
| AD 1 Temporal Ctx | 36.3 |
| AD 2 Temporal Ctx | 21.2 |
| AD 3 Temporal Ctx | 20.2 |
| AD 4 Temporal Ctx | 20.9 |
| AD 5 Inf Temporal Ctx | 50.0 |
| AD 5 Sup Temporal Ctx | 64.6 |
| AD 6 Inf Temporal Ctx | 58.6 |
| AD 6 Sup Temporal Ctx | 39.5 |
| Control 1 Temporal Ctx | 14.9 |
| Control 2 Temporal Ctx | 32.3 |
| Control 3 Temporal Ctx | 19.3 |
| Control 4 Temporal Ctx | 21.8 |
| Control (Path) 1 Temporal Ctx | 21.0 |
| Control (Path) 2 Temporal Ctx | 19.8 |
| Control (Path) 3 Temporal Ctx | 12.2 |
| Control (Path) 4 Temporal Ctx | 20.6 |
| AD 1 Occipital Ctx | 23.7 |
| AD 2 Occipital Ctx (Missing) | 0.0 |
| AD 3 Occipital Ctx | 18.8 |
| AD 4 Occipital Ctx | 18.8 |
| AD 5 Occipital Ctx | 13.8 |
| AD 6 Occipital Ctx | 28.3 |
| Control 1 Occipital Ctx | 12.0 |
| Control 2 Occipital Ctx | 39.0 |
| Control 3 Occipital Ctx | 23.0 |
| Control 4 Occipital Ctx | 18.6 |
| Control (Path) 1 Occipital Ctx | 39.2 |
| Control (Path) 2 Occipital Ctx | 8.6 |
| Control (Path) 3 Occipital Ctx | 10.3 |
| Control (Path) 4 Occipital Ctx | 9.8 |
| Control 1 Parietal Ctx | 17.2 |
| Control 2 Parietal Ctx | 69.3 |
| Control 3 Parietal Ctx | 17.9 |
| Control (Path) 1 Parietal Ctx | 42.0 |
| Control (Path) 2 Parietal Ctx | 20.0 |
| Control (Path) 3 Parietal Ctx | 11.0 |
| Control (Path) 4 Parietal Ctx | 11.2 |

TABLE KD
General_screening_panel_v1.5

| | Rel. Exp. (%) | Rel. Exp. (%) |
|---|---|---|
| | Ag5105, Run | Ag5105, Run |
| Tissue Name | 228969349 | 229514472 |
| Adipose | 1.9 | 1.4 |
| Melanoma* | 2.8 | 2.6 |
| Hs688(A).T | | |
| Melanoma* | 2.7 | 2.4 |
| Hs688(B).T | | |
| Melanoma* | 2.2 | 2.1 |
| M14 | | |
| Melanoma* | 9.9 | 10.7 |
| LOXIMVI | | |
| Melanoma* | 5.9 | 5.8 |
| SK-MEL-5 | | |
| Squamous | 4.0 | 2.8 |
| cell | | |
| carcinoma | | |
| SCC-4 | | |
| Testis Pool | 2.0 | 1.8 |
| Prostate ca.* | 33.9 | 42.9 |
| (bone met) | | |
| PC-3 | | |
| Prostate Pool | 1.8 | 1.8 |
| Placenta | 0.5 | 0.5 |
| Uterus Pool | 1.3 | 1.6 |
| Ovarian ca. | 1.8 | 2.1 |
| OVCAR-3 | | |
| Ovarian ca. | 7.2 | 9.9 |
| SK-OV-3 | | |
| Ovarian ca. | 1.2 | 2.2 |
| OVCAR-4 | | |
| Ovarian ca. | 17.0 | 21.3 |
| OVCAR-5 | | |
| Ovarian ca. | 13.2 | 16.7 |
| IGROV-1 | | |
| Ovarian ca. | 7.1 | 5.9 |
| OVCAR-8 | | |
| Ovary | 1.0 | 1.4 |
| Breast ca. | 1.5 | 1.6 |
| MCF-7 | | |
| Breast ca. | 5.1 | 5.4 |
| MDA-MB- | | |
| 231 | | |
| Breast ca. BT | 14.5 | 13.3 |
| 549 | | |
| Breast ca. | 0.1 | 0.0 |
| T47D | | |
| Breast ca. | 2.1 | 2.7 |
| MDA-N | | |
| Breast Pool | 2.6 | 2.1 |
| Trachea | 2.6 | 2.3 |
| Lung | 0.5 | 0.5 |
| Fetal Lung | 2.2 | 2.9 |
| Lung ca. | 0.1 | 0.1 |
| NCI-N417 | | |
| Lung ca. LX-1 | 18.2 | 20.0 |
| Lung ca. | 0.0 | 0.0 |
| NCI-H146 | | |
| Lung ca. | 0.7 | 0.6 |
| SHP-77 | | |
| Lung ca. | 33.4 | 36.9 |
| A549 | | |
| Lung ca. | 2.7 | 3.0 |
| NCI-H526 | | |
| Lung ca. | 3.1 | 3.2 |
| NCI-H23 | | |
| Lung ca. | 100.0 | 100.0 |
| NCI-H460 | | |
| Lung ca. | 6.0 | 6.0 |
| HOP-62 | | |
| Lung ca. | 3.8 | 4.9 |
| NCI-H522 | | |
| Liver | 0.2 | 0.2 |
| Fetal Liver | 3.3 | 3.7 |
| Liver ca. | 7.2 | 7.0 |
| HepG2 | | |
| Kidney Pool | 2.5 | 2.8 |
| Fetal Kidney | 2.0 | 2.0 |
| Renal ca. | 13.4 | 13.7 |
| 786-0 | | |
| Renal ca. | 2.3 | 2.2 |
| A498 | | |
| Renal ca. | 4.0 | 5.1 |
| ACHN | | |
| Renal ca. | 5.7 | 6.2 |
| UO-31 | | |
| Renal ca. TK-10 | 26.8 | 29.7 |
| Bladder | 2.9 | 3.6 |
| Gastric ca. (liver | 13.0 | 12.8 |
| met.) NCI-N87 | | |
| Gastric ca. | 14.4 | 17.2 |
| KATO III | | |
| Colon ca. SW- | 4.2 | 3.7 |
| 948 | | |
| Colon ca. | 11.3 | 10.3 |
| SW480 | | |
| Colon ca.* | 22.7 | 24.1 |
| (SW480 met) | | |
| SW620 | | |
| Colon ca. HT29 | 5.6 | 5.8 |
| Colon ca. HCT- | 9.5 | 11.9 |
| 116 | | |
| Colon ca. CaCo-2 | 15.5 | 18.3 |
| Colon cancer | 8.8 | 11.8 |
| tissue | | |
| Colon ca. | 1.9 | 1.0 |
| SW1116 | | |
| Colon ca. Colo- | 7.2 | 8.5 |
| 205 | | |
| Colon ca. SW-48 | 6.3 | 5.5 |
| Colon Pool | 1.7 | 1.7 |
| Small Intestine | 2.5 | 2.7 |
| Pool | | |
| Stomach Pool | 2.0 | 2.2 |
| Bone Marrow | 1.6 | 1.6 |
| Pool | | |
| Fetal Heart | 0.9 | 0.7 |
| Heart Pool | 0.3 | 0.8 |
| Lymph Node | 3.2 | 2.6 |
| Pool | | |
| Fetal Skeletal | 0.6 | 0.4 |
| Muscle | | |
| Skeletal Muscle | 0.6 | 1.1 |
| Pool | | |
| Spleen Pool | 0.9 | 1.1 |
| Thymus Pool | 2.0 | 2.3 |
| CNS cancer | 8.2 | 9.7 |
| (glio/astro) U87- | | |
| MG | | |
| CNS cancer | 12.2 | 13.6 |
| (glio/astro) U- | | |
| 118-MG | | |
| CNS cancer | 1.7 | 1.7 |
| (neuro; met) SK- | | |
| N-AS | | |
| CNS cancer | 1.5 | 1.8 |
| (astro) SF-539 | | |
| CNS cancer | 8.3 | 18.4 |
| (astro) SNB-75 | | |
| CNS cancer | 17.8 | 19.6 |
| (glio) SNB-19 | | |
| CNS cancer | 15.0 | 15.9 |
| (glio) SF-295 | | |
| Brain | 5.1 | 5.4 |
| (Amygdala) Pool | | |
| Brain | 7.5 | 10.2 |
| (cerebellum) | | |
| Brain (fetal) | 4.2 | 5.6 |
| Brain | 8.3 | 6.8 |
| (Hippocampus) | | |
| Pool | | |
| Cerebral Cortex | 6.5 | 5.3 |
| Pool | | |
| Brain (Substantia | 8.5 | 7.0 |
| nigra) Pool | | |
| Brain | 7.4 | 8.4 |
| (Thalamus) Pool | | |
| Brain (whole) | 6.3 | 6.3 |
| Spinal Cord Pool | 11.4 | 12.6 |
| Adrenal Gland | 0.9 | 1.0 |
| Pituitary gland | 0.3 | 0.2 |
| Pool | | |
| Salivary Gland | 1.6 | 1.7 |
| Thyroid (female) | 0.7 | 1.1 |
| Pancreatic ca. | 13.0 | 14.7 |
| CAPAN2 | | |
| Pancreas Pool | 2.9 | 3.8 |

CNS_neurodegeneration_v1.0 Summary: Ag5105 This panel confirms the expression of this gene at low levels in the brain in an independent group of individuals. This gene is found to be upregulated in the temporal cortex of Alzheimer's disease patients. Therefore, therapeutic modulation of the expression or function of this gene may decrease neuronal death and be of use in the treatment of this disease.

General_screening_panel_v1.4 Summary: Ag4986 Expression of this gene is low/undetectable in all samples on this panel (CTs>35). (Data not shown.)

General_screening_panel_v1.5 Summary: Ag5105 Two experiments with the same probe and primer set produce results that are in excellent agreement. Highest expression of this gene is seen in a breast cancer cell line (CTs=24-26). This gene is widely expressed in this panel, with high to moderate expression seen in brain, colon, gastric, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

This gene is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 5 Islet Summary: Ag4986 Expression of this gene is low/undetectable in all samples on this panel (CTs>35). (Data not shown.)

Panel 5D Summary: Ag5105 Results from one experiment with this gene are not included. The amp plot indicates that there were experimental difficulties with this run.

L. CG140316-01: Malic Enzyme Isoform1 (MB_X77244).

Expression of gene CG140316-01 was assessed using the primer-probe set Ag4998, described in Table LA. Results of the RTQ-PCR runs are shown in Tables LB and LC.

TABLE LA
Probe Name Ag4998

| | | | Start | SEQ ID |
|---|---|---|---|---|
| Primers | Sequences | Length | Position | No |
| Forward | 5′-agtttgcccatgaacatgaa-3′ | 20 | 1058 | 504 |
| Probe | TET-5′-gccattgttcaagaaataaaaccaactgc-3′-TAMRA | 29 | 1096 | 505 |
| Reverse | 5′-ttgcagcaactcctatgagg-3′ | 20 | 1125 | 506 |

TABLE LB
General_screening_panel_v1.4

| | Rel. Exp. (%) |
|---|---|
| | Ag4998, Run |
| Tissue Name | 219998185 |
| Adipose | 12.8 |
| Melanoma* Hs688(A).T | 15.8 |
| Melanoma* Hs688(B).T | 28.7 |
| Melanoma* M14 | 8.7 |
| Melanoma* LOXIMVI | 9.9 |
| Melanoma* SK-MEL-5 | 22.2 |
| Squamous cell carcinoma SCC-4 | 20.7 |
| Testis Pool | 7.2 |
| Prostate ca.* (bone met) PC-3 | 100.0 |
| Prostate Pool | 2.8 |
| Placenta | 0.2 |
| Uterus Pool | 0.9 |
| Ovarian ca. OVCAR-3 | 7.4 |
| Ovarian ca. SK-OV-3 | 37.6 |
| Ovarian ca. OVCAR-4 | 10.7 |
| Ovarian ca. OVCAR-5 | 6.9 |
| Ovarian ca. IGROV-1 | 4.0 |
| Ovarian ca. OVCAR-8 | 6.0 |
| Ovary | 6.4 |
| Breast ca. MCF-7 | 12.6 |
| Breast ca. MDA-MB-231 | 16.2 |
| Breast ca. BT 549 | 19.8 |
| Breast ca. T47D | 11.7 |
| Breast ca. MDA-N | 0.0 |
| Breast Pool | 3.1 |
| Trachea | 5.6 |
| Lung | 1.3 |
| Fetal Lung | 5.4 |
| Lung ca. NCI-N417 | 0.8 |
| Lung ca. LX-1 | 8.3 |
| Lung ca. NCI-H146 | 1.8 |
| Lung ca. SHP-77 | 30.8 |
| Lung ca. A549 | 67.4 |
| Lung ca. NCI-H526 | 1.7 |
| Lung ca. NCI-H23 | 6.2 |
| Lung ca. NCI-H460 | 55.9 |
| Lung ca. HOP-62 | 15.2 |
| Lung ca. NCI-H522 | 0.0 |
| Liver | 0.4 |
| Fetal Liver | 3.4 |
| Liver ca. HepG2 | 0.0 |
| Kidney Pool | 3.1 |
| Fetal Kidney | 0.8 |
| Renal ca. 786-0 | 14.7 |
| Renal ca. A498 | 14.2 |
| Renal ca. ACHN | 20.3 |
| Renal ca. UO-31 | 16.5 |
| Renal ca. TK- 10 | 7.6 |
| Bladder | 3.9 |
| Gastric ca. (liver met.)NCI-N87 | 11.7 |
| Gastric ca. KATO III | 36.3 |
| Colon ca. SW-948 | 12.5 |
| Colon ca. SW480 | 26.1 |
| Colon ca.* (SW480 met) SW620 | 12.2 |
| Colon ca. HT29 | 21.3 |
| Colon ca. HCT-116 | 59.0 |
| Colon ca. CaCo-2 | 56.3 |
| Colon cancer tissue | 7.9 |
| Colon ca. SW1116 | 4.9 |
| Colon ca. Colo-205 | 8.1 |
| Colon ca. SW-48 | 4.5 |
| Colon Pool | 4.2 |
| Small Intestine Pool | 1.0 |
| Stomach Pool | 1.9 |
| Bone Marrow Pool | 2.3 |
| Fetal Heart | 2.3 |
| Heart Pool | 2.0 |
| Lymph Node Pool | 3.0 |
| Fetal Skeletal Muscle | 0.0 |
| Skeletal Muscle Pool | 8.8 |
| Spleen Pool | 3.0 |
| Thymus Pool | 1.5 |
| CNS cancer (glio/astro) U87-MG | 0.0 |
| CNS cancer (glio/astro) U-118-MG | 10.7 |
| CNS cancer (neuro; met) SK-N-AS | 15.9 |
| CNS cancer (astro) SF-539 | 18.3 |
| CNS cancer (astro) SNB-75 | 0.1 |
| CNS cancer (glio) SNB-19 | 5.6 |
| CNS cancer (glio) SF-295 | 0.0 |
| Brain (Amygdala) Pool | 6.8 |
| Brain (cerebellum) | 4.6 |
| Brain (fetal) | 2.8 |
| Brain (Hippocampus) Pool | 6.3 |
| Cerebral Cortex Pool | 9.3 |
| Brain (Substantia nigra) Pool | 5.7 |
| Brain (Thalamus) Pool | 11.9 |
| Brain (whole) | 7.9 |
| Spinal Cord Pool | 7.4 |
| Adrenal Gland | 26.4 |
| Pituitary gland Pool | 3.6 |
| Salivary Gland | 0.6 |
| Thyroid (female) | 1.0 |
| Pancreatic ca. CAPAN2 | 9.3 |
| Pancreas Pool | 2.7 |

TABLE LC
Panel 5D

| | Rel. Exp. (%) |
|---|---|
| | Ag4998, Run |
| Tissue Name | 220259861 |
| 97457_Patient-02go_adipose | 8.5 |
| 97476_Patient-07sk_skeletal muscle | 5.2 |
| 97477_Patient-07ut_uterus | 14.0 |
| 97478_Patient-07pl_placenta | 2.4 |
| 97481_Patient-08sk_skeletal muscle | 7.1 |
| 97482_Patient-08ut_uterus | 9.7 |
| 97483_Patient-08pl_placenta | 1.4 |
| 97486_Patient-09sk_skeletal muscle | 6.9 |
| 97487_Patient-09ut_uterus | 16.0 |
| 97488_Patient-09pl_placenta | 1.2 |
| 97492_Patient-10ut_uterus | 9.0 |
| 97493_Patient-10pl_placenta | 3.5 |
| 97495_Patient-11go_adipose | 5.9 |
| 97496_Patient-11sk_skeletal muscle | 16.2 |
| 97497_Patient-11ut_uterus | 23.0 |
| 97498_Patient-11pl_placenta | 0.0 |
| 97500_Patient-12go_adipose | 28.9 |
| 97501_Patient-12sk_skeletal muscle | 33.9 |
| 97502_Patient-12ut_uterus | 15.4 |
| 97503_Patient-12pl_placenta | 0.3 |
| 94721_Donor 2 U - A_Mesenchymal Stem Cells | 10.2 |
| 94722_Donor 2 U - B_Mesenchymal Stem Cells | 36.1 |
| 94723_Donor 2 U - C_Mesenchymal Stem Cells | 9.0 |
| 94709_Donor 2 AM - A_adipose | 26.4 |
| 94710_Donor 2 AM - B_adipose | 11.7 |
| 94711_Donor 2 AM - C_adipose | 9.0 |
| 94712_Donor 2 AD - A_adipose | 77.4 |
| 94713_Donor 2 AD - B_adipose | 94.6 |
| 94714_Donor 2 AD - C_adipose | 100.0 |
| 94742_Donor 3 U - A_Mesenchymal Stem Cells | 6.7 |
| 94743_Donor 3 U - B_Mesenchymal Stem Cells | 12.4 |
| 94730_Donor 3 AM - A_adipose | 20.2 |
| 94731_Donor 3 AM - B_adipose | 16.6 |
| 94732_Donor 3 AM - C_adipose | 16.5 |
| 94733_Donor 3 AD - A_adipose | 92.7 |
| 94734_Donor 3 AD - B_adipose | 55.1 |
| 94735_Donor 3 AD - C_adipose | 57.8 |
| 77138_Liver_HepG2untreated | 8.7 |
| 73556_Heart_Cardiac stromal cells (primary) | 9.0 |
| 81735_Small Intestine | 5.0 |
| 72409_Kidney_Proximal Convoluted Tubule | 12.3 |
| 82685_Small intestine_Duodenum | 18.8 |
| 90650_Adrenal_Adrenocortical adenoma | 9.5 |
| 72410_Kidney_HRCE | 33.9 |
| 72411_Kidney_HRE | 25.3 |
| 73139_Uterus_Uterine smooth muscle cells | 19.2 |

General_screening_panel_v1.4 Summary: Ag4998 Cytosolic malic enzyme is ubiquitously expressed including endocrine/metabolically-relevant tissues such as, adipose, GI, liver, and skeletal muscle. These results indicate that this enzyme is critical for normal physiology. Furthermore, disregulated expression of this gene may contribute to neuroendocrine disorders or metabolic diseases, such as obesity and diabetes.

Highest expression of this gene is seen in a prostate cancer cell line (CT=25.4). This gene is widely expressed in this panel, with high to moderate expression seen in brain, colon, gastric, lung, breast, ovarian, and melanoma cancer cell lines. This expression profile suggests a role for this gene product in cell survival and proliferation. Modulation of this gene product may be useful in the treatment of cancer.

This gene is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 5D Summary: Ag4998 Cytosolic malic enzyme has low to moderate expression in fully differentiated adipose, and adipose found in diabetic gestational diabetics.

M. CG142427-01: ATP Citrate Lyase.

Expression of gene CG142427-01 and CG142404-01 were assessed using the primer-probe set Ag6008, described in Table MA. Results of the RTQ-PCR runs are shown in Tables MB and MC.

TABLE MA
Probe Name Ag6008

| | | | Start | SEQ ID |
|---|---|---|---|---|
| Primers | Sequences | Length | Position | No |
| Forward | 5′-agattacgtcaggcagcactt-3′ | 21 | 3113 | 507 |
| Probe | TET-5′-cactcctctgctcgattatgcactgg-3′-TAMRA | 26 | 3140 | 508 |
| Reverse | 5′-gcttcttcgaggtggtaatctt-3′ | 22 | 3174 | 509 |

TABLE MB
General_screening_panel_v1 .5

| | Rel. Exp. (%) |
|---|---|
| | Ae6008. Run |
| Tissue Name | 228763479 |
| Adipose | 6.2 |
| Melanoma* Hs688(A).T | 37.6 |
| Melanoma* Hs688(B).T | 59.0 |
| Melanoma* M14 | 55.9 |
| Melanoma* LOXIMVI | 59.0 |
| Melanoma* SK-MEL-5 | 41.8 |
| Squamous cell carcinoma SCC-4 | 24.1 |
| Testis Pool | 6.0 |
| Prostate ca.* (bone met) PC-3 | 32.8 |
| Prostate Pool | 13.0 |
| Placenta | 6.1 |
| Uterus Pool | 6.6 |
| Ovarian ca. OVCAR-3 | 12.9 |
| Ovarian ca. SK-OV-3 | 47.3 |
| Ovarian ca. OVCAR-4 | 17.2 |
| Ovarian ca. OVCAR-5 | 35.1 |
| Ovarian ca. IGROV-1 | 22.2 |
| Ovarian ca. OVCAR-8 | 8.2 |
| Ovary | 8.0 |
| Breast ca. MCF-7 | 23.7 |
| Breast ca. MDA-MB-231 | 46.7 |
| Breast ca. BT 549 | 60.7 |
| Breast ca. T47D | 29.1 |
| Breast ca. MDA-N | 12.9 |
| Breast Pool | 8.0 |
| Trachea | 9.3 |
| Lung | 1.4 |
| Fetal Lung | 16.3 |
| Lung ca. NCI-N417 | 30.1 |
| Lung ca. LX-1 | 28.1 |
| Lung ca. NCI-H146 | 23.5 |
| Lung ca. SHP-77 | 46.7 |
| Lung ca. A549 | 100.0 |
| Lung ca. NCI-H526 | 10.0 |
| Lung ca. NCI-H23 | 23.5 |
| Lung ca. NCI-H460 | 25.5 |
| Lung ca. HOP-62 | 29.5 |
| Lung ca. NCI-H522 | 57.4 |
| Liver | 0.8 |
| Fetal Liver | 22.4 |
| Liver ca. HepG2 | 23.0 |
| Kidney Pool | 7.5 |
| Fetal Kidney | 5.4 |
| Renal ca. 786-0 | 36.3 |
| Renal ca. A498 | 33.0 |
| Renal ca. ACHN | 80.7 |
| Renal ca. UO-31 | 31.9 |
| Renal ca. TK-10 | 64.2 |
| Bladder | 12.4 |
| Gastric ca. (liver met.) NCI-N87 | 65.1 |
| Gastric ca. KATO III | 59.5 |
| Colon ca. SW-948 | 14.5 |
| Colon ca. SW480 | 62.4 |
| Colon ca.* (SW480 met) SW620 | 32.3 |
| Colon ca. HT29 | 27.4 |
| Colon ca. HCT-116 | 45.7 |
| Colon ca. CaCo-2 | 66.0 |
| Colon cancer tissue | 8.3 |
| Colon ca. SW1116 | 4.0 |
| Colon ca. Colo-205 | 11.1 |
| Colon ca. SW-48 | 14.9 |
| Colon Pool | 13.3 |
| Small Intestine Pool | 5.6 |
| Stomach Pool | 4.0 |
| Bone Marrow Pool | 3.8 |
| Fetal Heart | 3.5 |
| Heart Pool | 2.5 |
| Lymph Node Pool | 8.4 |
| Fetal Skeletal Muscle | 3.7 |
| Skeletal Muscle Pool | 3.4 |
| Spleen Pool | 5.3 |
| Thymus Pool | 6.8 |
| CNS cancer (glio/astro) U87-MG | 60.7 |
| CNS cancer (glio/astro) U-118-MG | 59.0 |
| CNS cancer (neuro; met) SK-N-AS | 60.7 |
| CNS cancer (astro) SF-539 | 24.8 |
| CNS cancer (astro) SNB-75 | 32.5 |
| CNS cancer (glio) SNB-19 | 25.2 |
| CNS cancer (glio) SF-295 | 76.8 |
| Brain (Amygdala) Pool | 4.8 |
| Brain (cerebellum) | 28.3 |
| Brain (fetal) | 16.5 |
| Brain (Hippocampus) Pool | 8.6 |
| Cerebral Cortex Pool | 10.5 |
| Brain (Substantia nigra) Pool | 6.3 |
| Brain (Thalamus) Pool | 10.7 |
| Brain (whole) | 12.2 |
| Spinal Cord Pool | 7.4 |
| Adrenal Gland | 13.2 |
| Pituitary gland Pool | 1.9 |
| Salivary Gland | 4.0 |
| Thyroid (female) | 2.7 |
| Pancreatic ca. CAPAN2 | 36.3 |
| Pancreas Pool | 11.2 |

TABLE MC
Panel 5 Islet

| | Rel. Exp. (%) |
|---|---|
| | Ag6008, Run |
| Tissue Name | 245239907 |
| 97457_Patient-02go_adipose | 12.6 |
| 97476_Patient-07sk_skeletal muscle | 9.5 |
| 97477_Patient-07ut_uterus | 8.4 |
| 97478_Patient-07pl_placenta | 16.4 |
| 99167_Bayer Patient 1 | 70.7 |
| 97482_Patient-08ut_uterus | 7.9 |
| 97483_Patient-08pl_placenta | 15.6 |
| 97486_Patient-09sk_skeletal muscle | 0.6 |
| 97487_Patient-09ut_uterus | 3.6 |
| 97488_Patient-09pl_placenta | 9.6 |
| 97492_Patient-10ut_uterus | 9.9 |
| 97493_Patient-10pl_placenta | 18.3 |
| 97495_Patient-11go_adipose | 5.5 |
| 97496_Patient-11sk_skeletal muscle | 0.4 |
| 97497_Patient-11ut_uterus | 3.5 |
| 97498_Patient-11pl_placenta | 11.0 |
| 97500_Patient-12go_adipose | 7.4 |
| 97501_Patient-12sk_skeletal muscle | 6.9 |
| 97502_Patient-12ut_uterus | 9.3 |
| 97503_Patient-12pl_placenta | 6.1 |
| 94721_Donor 2 U - A_Mesenchymal Stem Cells | 6.7 |
| 94722_Donor 2 U - B_Mesenchymal Stem Cells | 13.6 |
| 94723_Donor 2 U - C_Mesenchymal Stem Cells | 8.9 |
| 94709_Donor 2 AM - A_adipose | 26.8 |
| 94710_Donor 2 AM - B_adipose | 26.4 |
| 94711_Donor 2 AM - C_adipose | 8.4 |
| 94712_Donor 2 AD - A_adipose | 37.6 |
| 94713_Donor 2 AD - B_adipose | 31.0 |
| 94714_Donor 2 AD - C_adipose | 59.0 |
| 94742_Donor 3 U - A_Mesenchymal Stem Cells | 11.0 |
| 94743_Donor 3 U - B_Mesenchymal Stem Cells | 34.2 |
| 94730_Donor 3 AM - A_adipose | 60.3 |
| 94731_Donor 3 AM - B_adipose | 27.4 |
| 94732_Donor 3 AM - C_adipose | 42.3 |
| 94733_Donor 3 AD - A_adipose | 100.0 |
| 94734_Donor 3 AD - B_adipose | 44.1 |
| 94735_Donor 3 AD - C_adipose | 84.1 |
| 77138_Liver_HepG2untreated | 0.0 |
| 73556_Heart_Cardiac stromal cells (primary) | 14.8 |
| 81735_Small Intestine | 9.5 |
| 72409_Kidney_Proximal Convoluted Tubule | 24.5 |
| 82685_Small intestine_Duodenum | 7.1 |
| 90650_Adrenal_Adrenocortical adenoma | 2.4 |
| 72410_Kidney_HRCE | 65.5 |
| 72411_Kidney_HRE | 46.0 |
| 73139_Uterus_Uterine smooth muscle cells | 30.4 |

General_screening_panel_v1.5 Summary: Ag6008 Highest expression of this gene is detected in a lung cancer A549 cell line (CT=22.4). High expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers.

Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at high levels in pancreas, adipose, adrenal gland, thyroid, pituitary gland, skeletal muscle, heart, liver and the gastrointestinal tract. Therefore, therapeutic modulation of the activity of this gene through the use of small molecule drug may prove useful in the treatment of endocrine/metabolically related diseases, such as obesity and diabetes.

Interestingly, this gene is expressed at much higher levels in fetal (CTs=24-25), when compared to adult liver and lung (CTs=28-29). This observation suggests that expression of this gene can be used to distinguish fetal from adult lung and liver. In addition, the relative overexpression of this gene in fetal tissue suggests that the protein product may enhance lung and liver growth or development in the fetus and thus may also act in a regenerative capacity in the adult. Therefore, therapeutic modulation of the protein encoded by this gene could be useful in treatment of lung and liver related diseases.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Panel 5 Islet Summary: Ag6008 Highest expression of this gene is detected in differentiated adipose (CT=27.7). This gene shows widespread expression in this panel. Moderate to high expression of this gene is detected in the tissues with metabolic/endocrine functions including islet cells, adipose, skeletal muscle, and gastrointestinal tracts.

This gene codes for ATP-citrate lyase. It is a major source of acetyl CoA that is the building block of lipid biosynthesis and provides substrate for the production of cholesterol. Reduced flux of acetyl CoA through the cholesterol biosynthetic pathway will prevent excess production of LXR alpha ligands. LXR alpha is a nuclear hormone receptor that is abundantly expressed in tissues associated with lipid metabolism. Activation of LXR alpha leads to the up-regulation of fatty acid synthesis. Thus, ATP-citrate lyase may be a target for the treatment and/or prevention of obesity because its inhibition will decrease the availability of acetyl CoA for the synthesis of LXR alpha ligands, fatty acids, and triglycerides.

References:

1. Chawla A, Repa J J, Evans R M, Mangelsdorf D J. Nuclear receptors and lipid physiology: opening the X-files. Science. Nov. 30, 2001;294(5548):1866-70. Review. PMID: 11729302.

2. Moon Y A, Lee J J, Park S W, Ahn Y H, Kim K S. The roles of sterol regulatory element-binding proteins in the transactivation of the rat ATP citrate-lyase promoter. J Biol Chem. Sep. 29, 2000;275(39):30280-6. PMID: 10801800.

3. Sato R. Okamoto A, Inoue J. Miyamoto W. Sakai Y. Emoto N. Shimano H. Maeda M. Transcriptional regulation of the ATP citrate-lyase gene by sterol regulatory element-binding proteins. J Biol Chem. Apr. 28, 2000;275(17):12497-502. PMID: 10777536.

N. CG142631-01: Serine Dehydratase.

Expression of gene CG14263 1-01 was assessed using the primer-probe set Ag6006, described in Table NA. Results of the RTQ-PCR runs are shown in Tables NB, NC, ND and NE.

TABLE NA
Probe Name Ag6006

| | | | Start | SEQ ID |
|---|---|---|---|---|
| Primers | Sequences | Length | Position | No |
| Forward | 5′-aagttcgtggatgatgagaaga-3′ | 22 | 858 | 510 |
| Probe | TET-5′-ctggccgctgtctatagccacgt-3′-TAMRA | 23 | 909 | 511 |
| Reverse | 5′-tccagttggagcttctggat-3′ | 20 | 933 | 512 |

TABLE NB
General_screening_panel_v1.5

| | Rel. Exp. (%) | Rel. Exp. (%) |
|---|---|---|
| | Ag6006, Run | Ag6006, Run |
| Tissue Name | 228738305 | 228763464 |
| Adipose | 2.8 | 3.1 |
| Melanoma* | 0.0 | 0.0 |
| Hs688(A).T | | |
| Melanoma* | 0.0 | 0.0 |
| Hs688(B).T | | |
| Melanoma* | 0.0 | 0.0 |
| M14 | | |
| Melanoma* | 0.0 | 0.0 |
| LOXIMVI | | |
| Melanoma* | 0.0 | 0.0 |
| SK-MEL-5 | | |
| Squamous | 0.0 | 0.0 |
| cell | | |
| carcinoma | | |
| SCC-4 | | |
| Testis Pool | 0.1 | 0.1 |
| Prostate ca.* | 0.0 | 0.0 |
| (bone met) | | |
| PC-3 | | |
| Prostate Pool | 0.2 | 0.1 |
| Placenta | 0.5 | 0.2 |
| Uterus Pool | 0.1 | 0.2 |
| Ovarian ca. | 0.7 | 0.3 |
| OVCAR-3 | | |
| Ovarian ca. | 0.0 | 0.0 |
| SK-OV-3 | | |
| Ovarian ca. | 0.0 | 0.0 |
| OVCAR-4 | | |
| Ovarian ca. | 0.1 | 0.3 |
| OVCAR-5 | | |
| Ovarian ca. | 0.0 | 0.0 |
| IGROV-1 | | |
| Ovarian ca. | 0.1 | 0.0 |
| OVCAR-8 | | |
| Ovary | 0.6 | 0.6 |
| Breast ca. | 0.0 | 0.0 |
| MCF-7 | | |
| Breast ca. | 0.0 | 0.0 |
| MDA-MB- | | |
| 231 | | |
| Breast ca. BT | 0.0 | 0.1 |
| 549 | | |
| Breast ca. | 0.0 | 0.0 |
| T47D | | |
| Breast ca. | 0.0 | 0.0 |
| MDA-N | | |
| Breast Pool | 0.3 | 0.0 |
| Trachea | 1.2 | 1.5 |
| Lung | 0.0 | 0.0 |
| Fetal Lung | 0.9 | 1.8 |
| Lung ca. | 0.0 | 0.0 |
| NCI-N417 | | |
| Lung ca. LX-1 | 0.0 | 0.0 |
| Lung ca. | 0.0 | 0.0 |
| NCI-H146 | | |
| Lung ca. | 0.1 | 0.0 |
| SHP-77 | | |
| Lung ca. | 1.7 | 1.4 |
| A549 | | |
| Lung ca. | 0.0 | 0.0 |
| NCI-H526 | | |
| Lung ca. | 0.0 | 0.0 |
| NCI-H23 | | |
| Lung ca. | 0.0 | 0.0 |
| NCI-H460 | | |
| Lung ca. | 0.0 | 0.0 |
| HOP-62 | | |
| Lung ca. | 0.0 | 0.1 |
| NCI-H522 | | |
| Liver | 100.0 | 100.0 |
| Fetal Liver | 0.9 | 0.8 |
| Liver ca. | 0.0 | 0.0 |
| HepG2 | | |
| Kidney Pool | 0.1 | 0.1 |
| Fetal Kidney | 0.0 | 0.0 |
| Renal ca. | 0.2 | 0.1 |
| 786-0 | | |
| Renal ca. | 0.0 | 0.1 |
| A498 | | |
| Renal ca. | 0.0 | 0.0 |
| ACHN | | |
| Renal ca. | 0.0 | 0.0 |
| UO-31 | | |
| Renal ca. TK-10 | 12.9 | 12.4 |
| Bladder | 5.4 | 7.6 |
| Gastric ca. (liver | 1.1 | 0.9 |
| met.) NCI-N87 | | |
| Gastric ca. | 0.0 | 0.0 |
| KATO III | | |
| Colon ca. SW- | 0.0 | 0.0 |
| 948 | | |
| Colon ca. | 0.0 | 0.0 |
| SW480 | | |
| Colon ca.* | 0.0 | 0.0 |
| (SW480 met) | | |
| SW620 | | |
| Colon ca. HT29 | 0.0 | 0.0 |
| Colon ca. HCT- | 0.0 | 0.0 |
| 116 | | |
| Colon ca. CaCo-2 | 0.1 | 0.0 |
| Colon cancer | 22.5 | 27.4 |
| tissue | | |
| Colon ca. | 0.0 | 0.0 |
| SW1116 | | |
| Colon ca. Colo- | 0.0 | 0.0 |
| 205 | | |
| Colon ca. SW-48 | 0.0 | 0.0 |
| Colon Pool | 0.1 | 0.3 |
| Small Intestine | 0.0 | 0.1 |
| Pool | | |
| Stomach Pool | 1.5 | 1.2 |
| Bone Marrow | 0.1 | 0.1 |
| Pool | | |
| Fetal Heart | 0.0 | 0.0 |
| Heart Pool | 0.0 | 0.3 |
| Lymph Node | 0.0 | 0.0 |
| Pool | | |
| Fetal Skeletal | 0.0 | 0.0 |
| Muscle | | |
| Skeletal Muscle | 0.0 | 0.0 |
| Pool | | |
| Spleen Pool | 1.2 | 0.6 |
| Thymus Pool | 0.2 | 0.0 |
| CNS cancer | 0.0 | 0.0 |
| (glio/astro) U87- | | |
| MG | | |
| CNS cancer | 0.1 | 0.0 |
| (glio/astro) U- | | |
| 118-MG | | |
| CNS cancer | 0.0 | 0.0 |
| (neuro; met) SK- | | |
| N-AS | | |
| CNS cancer | 0.2 | 0.0 |
| (astro) SF-539 | | |
| CNS cancer | 0.1 | 0.0 |
| (astro) SNB-75 | | |
| CNS cancer | 0.0 | 0.0 |
| (glio) SNB-19 | | |
| CNS cancer | 0.0 | 0.2 |
| (glio) SF-295 | | |
| Brain | 3.8 | 2.9 |
| (Amygdala) Pool | | |
| Brain | 7.9 | 10.2 |
| (cerebellum) | | |
| Brain (fetal) | 0.5 | 0.6 |
| Brain | 3.7 | 5.9 |
| (Hippocampus) | | |
| Pool | | |
| Cerebral Cortex | 2.2 | 2.4 |
| Pool | | |
| Brain (Substantia | 3.1 | 3.3 |
| nigra) Pool | | |
| Brain | 3.4 | 3.5 |
| (Thalamus) Pool | | |
| Brain (whole) | 4.8 | 3.2 |
| Spinal Cord Pool | 2.0 | 1.8 |
| Adrenal Gland | 13.2 | 12.7 |
| Pituitary gland | 0.0 | 0.0 |
| Pool | | |
| Salivary Gland | 0.2 | 0.2 |
| Thyroid (female) | 0.4 | 0.7 |
| Pancreatic ca. | 0.0 | 0.0 |
| CAPAN2 | | |
| Pancreas Pool | 0.3 | 0.3 |

TABLE NC
Oncology_cell_line_screening_panel_v3.1

| | Rel. Exp. (%) | Rel. Exp. (%) |
|---|---|---|
| | Ag6006, Run | Ag6006, Run |
| Tissue Name | 225138976 | 230277129 |
| Daoy | 0.0 | 0.0 |
| Medulloblastoma/Cerebellum | | |
| TE671 | 0.0 | 0.0 |
| Medulloblastom/Cerebellum | | |
| D283 Med | 0.0 | 0.0 |
| Medulloblastoma/Cerebellum | | |
| PFSK-1 Primitive | 13.3 | 3.1 |
| Neuroectodermal/Cerebellum | | |
| XF-498_CNS | 0.0 | 0.0 |
| SNB-78_CNS/glioma | 0.0 | 0.0 |
| SF-268_CNS/glioblastoma | 0.0 | 0.0 |
| T98G_Glioblastoma | 0.0 | 0.0 |
| SK-N-SH_Neuroblastoma | 0.0 | 0.0 |
| (metastasis) | | |
| SF-295_CNS/glioblastoma | 0.0 | 0.0 |
| Cerebellum | 66.9 | 97.9 |
| Cerebellum | 100.0 | 100.0 |
| NCI-H292_Mucoepidermoid | 0.0 | 0.0 |
| lung ca. | | |
| DMS-114_Small cell lung | 0.0 | 0.0 |
| cancer | | |
| DMS-79_Small cell lung | 0.0 | 0.0 |
| cancer/neuroendocrine | | |
| NCI-H146_Small cell lung | 0.0 | 0.0 |
| cancer/neuroendocrine | | |
| NCI-H526_Small cell lung | 0.0 | 0.0 |
| cancer/neuroendocrine | | |
| NCI-N417_Small cell lung | 0.0 | 0.0 |
| cancer/neuroendocrine | | |
| NCI-H82_Small cell lung | 3.7 | 0.0 |
| cancer/neuroendocrine | | |
| NCI-H157_Squamous cell | 0.0 | 0.0 |
| lung cancer (metastasis) | | |
| NCI-H1155_Large cell lung | 0.0 | 0.0 |
| cancer/neuroendocrine | | |
| NCI-H1299_Large cell lung | 0.0 | 0.0 |
| cancer/neuroendocrine | | |
| NCI-H727_Lung carcinoid | 0.0 | 0.0 |
| NCI-UMC-11_Lung | 0.0 | 0.0 |
| carcinoid | | |
| LX-1_Small cell lung cancer | 0.0 | 0.0 |
| Colo-205_Colon cancer | 0.0 | 0.0 |
| KM12_Colon cancer | 0.0 | 0.0 |
| KM20L2_Colon cancer | 0.0 | 0.0 |
| NCI-H716_Colon cancer | 0.0 | 0.0 |
| SW-48_Colon | 0.0 | 0.0 |
| adenocarcinoma | | |
| SW1116_Colon | 0.0 | 0.0 |
| adenocarcinoma | | |
| LS 174T_Colon | 0.0 | 0.0 |
| adenocarcinoma | | |
| SW-948_Colon | 0.0 | 0.0 |
| adenocarcinoma | | |
| SW-480_Colon | 0.0 | 0.0 |
| adenocarcinoma | | |
| NCI-SNU-5_Gastric ca. | 0.0 | 0.0 |
| KATO III_Stomach | 0.5 | 0.0 |
| NCI-SNU-16_Gastric ca. | 2.6 | 0.0 |
| NCI-SNU-1_Gastric ca. | 0.0 | 0.0 |
| RF-1_Gastric | 7.4 | 11.3 |
| adenocarcinoma | | |
| RF-48_Gastric | 17.1 | 7.8 |
| adenocarcinoma | | |
| MKN-45_Gastric ca. | 0.0 | 0.0 |
| NCI-N87_Gastric ca. | 0.0 | 0.0 |
| OVCAR-5_Ovarian ca. | 0.0 | 0.0 |
| RL95-2_Uterine carcinoma | 2.0 | 0.0 |
| HelaS3_Cervical | 0.0 | 0.0 |
| adenocarcinoma | | |
| Ca Ski_Cervical | 0.0 | 0.0 |
| epidermoid | | |
| carcinoma | | |
| (metastasis) | | |
| ES-2_Ovarian clear | 0.0 | 0.0 |
| cell carcinoma | | |
| Ramos/6 h stim_{—} | 0.0 | 0.0 |
| Stimulated with | | |
| PMA/ionomycin 6 h | | |
| Ramos/14 h stim_{—} | 0.0 | 0.0 |
| Stimulated with | | |
| PMA/ionomycin 14 h | | |
| MEG-01_Chronic | 2.2 | 6.9 |
| myelogenous | | |
| leukemia | | |
| (megokaryoblast) | | |
| Raji_Burkitt's | 0.0 | 0.0 |
| lymphoma | | |
| Daudi_Burkitt's | 0.0 | 0.0 |
| lymphoma | | |
| U266_B-cell | 0.0 | 3.8 |
| plasmacytoma/mye- | | |
| loma | | |
| CA46_Burkitt's | 0.0 | 0.0 |
| lymphoma | | |
| RL_non-Hodgkin's | 0.0 | 0.0 |
| B-cell lymphoma | | |
| JM1_pre-B-cell | 0.0 | 0.0 |
| lymphoma/leukemia | | |
| Jurkat_T cell | 0.0 | 0.0 |
| leukemia | | |
| TF- | 12.2 | 10.4 |
| 1_Erythroleukemia | | |
| HUT 78_T-cell | 0.0 | 0.0 |
| lymphoma | | |
| U937_Histiocytic | 43.5 | 42.3 |
| lymphoma | | |
| KU- | 2.3 | 0.0 |
| 812_Myelogenous | | |
| leukemia | | |
| 769-P_Clear cell | 0.0 | 0.0 |
| renal ca. | | |
| Caki-2_Clear cell | 0.0 | 0.0 |
| renal ca. | | |
| SW 839_Clear cell | 0.0 | 0.0 |
| renal ca. | | |
| G401_Wilms' tumor | 8.3 | 20.7 |
| Hs766T_Pancreatic | 2.0 | 0.0 |
| ca. (LN metastasis) | | |
| CAPAN- | 0.0 | 0.0 |
| 1_Pancreatic | | |
| adenocarcinoma | | |
| (liver metastasis) | | |
| SU86.86_Pancreatic | 0.0 | 0.0 |
| carcinoma (liver | | |
| metastasis) | | |
| BxPC-3_Pancreatic | 0.0 | 0.0 |
| adenocarcinoma | | |
| HPAC_Pancreatic | 0.0 | 0.0 |
| adenocarcinoma | | |
| MIA PaCa- | 0.0 | 0.0 |
| 2_Pancreatic ca. | | |
| CFPAC-1_Pancreatic | 0.6 | 0.0 |
| ductal | | |
| adenocarcinoma | | |
| PANC-1_Pancreatic | 0.0 | 0.0 |
| epithelioid ductal ca. | | |
| T24_Bladder ca. | 0.0 | 0.0 |
| (transitional cell) | | |
| 5637_Bladder ca. | 0.0 | 0.0 |
| HT-1197_Bladder ca. | 2.3 | 0.0 |
| UM-UC-3_Bladder | 0.0 | 0.0 |
| ca. (transitional cell) | | |
| A204_Rhabdomyo- | 0.0 | 0.0 |
| sarcoma | | |
| HT- | 0.0 | 2.0 |
| 1080_Fibrosarcoma | | |
| MG- | 0.0 | 8.0 |
| 63_Osteosarcoma | | |
| (bone) | | |
| SK-LMS- | 3.7 | 0.0 |
| 1_Leiomyosarcoma | | |
| (vulva) | | |
| SJRH30_Rhabdomyo- | 0.0 | 0.0 |
| sarcoma (met to bone | | |
| marrow) | | |
| A431_Epidermoid | 1.5 | 0.0 |
| ca. | | |
| WM266- | 1.6 | 3.8 |
| 4_Melanoma | | |
| DU 145_Prostate | 0.0 | 0.0 |
| MDA-MB- | 2.4 | 0.0 |
| 468_Breast | | |
| adenocarcinoma | | |
| SSC-4_Tongue | 0.0 | 0.0 |
| SSC-9_Tongue | 0.0 | 0.0 |
| SSC-15_Tongue | 0.0 | 0.0 |
| CAL 27_Squamous | 0.0 | 0.0 |
| cell ca. of tongue | | |

TABLE ND
Panel 4.1D

| | Rel. Exp. (%) |
|---|---|
| | Ag6006, Run |
| Tissue Name | 225787022 |
| Secondary Th1 act | 0.0 |
| Secondary Th2 act | 0.0 |
| Secondary Tr1 act | 0.0 |
| Secondary Th1 rest | 0.2 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 7.5 |
| LAK cells IL-2 | 0.0 |
| LAK cells IL-2 + IL-12 | 0.2 |
| LAK cells IL-2 + IFN gamma | 0.2 |
| LAK cells IL-2 + IL-18 | 0.0 |
| LAK cells PMA/ionomycin | 3.6 |
| NK Cells IL-2 rest | 0.0 |
| Two Way MLR 3 day | 1.3 |
| Two Way MLR 5 day | 1.3 |
| Two Way MLR 7 day | 1.1 |
| PBMC rest | 0.5 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 8.1 |
| Dendritic cells LPS | 10.4 |
| Dendritic cells anti-CD40 | 7.1 |
| Monocytes rest | 0.4 |
| Monocytes LPS | 16.0 |
| Macrophages rest | 87.7 |
| Macrophages LPS | 82.4 |
| HUVEC none | 0.0 |
| HUVEC starved | 0.0 |
| HUVEC IL-lbeta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.4 |
| Lung Microvascular EC none | 0.6 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.2 |
| KU-812 (Basophil) PMA/ionomycin | 0.2 |
| CCD1106 (Keratinocytes) none | 0.0 |
| CCD1106 (Keratinocytes) TNFalpha + IL-1beta | 0.0 |
| Liver cirrhosis | 100.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.2 |
| Lung fibroblast IL-4 | 0.3 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 0.1 |
| Lung | 1.2 |
| Thymus | 3.2 |
| Kidney | 2.5 |

TABLE NE
Panel 5 Islet

| | Rel. Exp. (%) | Rel. Exp. (%) | Rel. Exp. (%) |
|---|---|---|---|
| | Ag6006, Run | Ag6006, Run | Ag6006, Run |
| Tissue Name | 225051164 | 248989152 | 249139055 |
| 97457_Patient- | 6.5 | 0.0 | 20.0 |
| 02go_adipose | | | |
| 97476_Patient- | 20.7 | 0.0 | 15.6 |
| 07sk_skeletal | | | |
| muscle | | | |
| 97477_Patient- | 6.7 | 0.0 | 0.0 |
| 07ut_uterus | | | |
| 97478_Patient- | 11.8 | 0.0 | 5.0 |
| 07pl_placenta | | | |
| 99167_Bayer | 88.3 | 100.0 | 62.0 |
| Patient 1 | | | |
| 97482_Patient- | 8.5 | 6.7 | 0.0 |
| 08ut_uterus | | | |
| 97483_Patient- | 4.4 | 13.5 | 5.4 |
| 08pl_placenta | | | |
| 97486_Patient- | 0.0 | 0.0 | 0.0 |
| 09sk_skeletal | | | |
| muscle | | | |
| 97487_Patient- | 0.0 | 0.0 | 0.0 |
| 09ut_uterus | | | |
| 97488_Patient- | 4.9 | 0.0 | 0.0 |
| 09pl_placenta | | | |
| 97492_Patient- | 0.0 | 0.0 | 0.0 |
| 10ut_uterus | | | |
| 97493_Patient- | 4.6 | 0.0 | 5.1 |
| 10pl_placenta | | | |
| 97495_Patient- | 0.0 | 0.0 | 3.8 |
| 11go_adipose | | | |
| 97496_Patient- | 0.0 | 0.0 | 0.0 |
| 11sk_skeletal | | | |
| muscle | | | |
| 97497_Patient- | 0.0 | 0.0 | 0.0 |
| 11ut_uterus | | | |
| 97498_Patient- | 0.0 | 0.0 | 0.0 |
| 11pl_placenta | | | |
| 97500_Patient- | 0.0 | 6.0 | 4.9 |
| 12go_adipose | | | |
| 97501_Patient- | 4.0 | 0.0 | 9.2 |
| 12sk_skeletal | | | |
| muscle | | | |
| 97502_Patient- | 0.0 | 5.1 | 0.0 |
| 12ut_uterus | | | |
| 97503_Patient- | 14.9 | 7.3 | 7.7 |
| 12pl_placenta | | | |
| 94721_Donor 2 | 0.0 | 0.0 | 0.0 |
| U - | | | |
| A_Mesenchymal | | | |
| Stem Cells | | | |
| 94722_Donor 2 | 0.0 | 0.0 | 0.0 |
| U - | | | |
| B_Mesenchymal | | | |
| Stem Cells | | | |
| 94723_Donor 2 | 0.0 | 0.0 | 2.4 |
| U - | | | |
| C_Mesenchymal | | | |
| Stem Cells | | | |
| 94709_Donor 2 | 0.0 | 0.0 | 0.0 |
| AM - A_adipose | | | |
| 94710_Donor 2 | 0.0 | 0.0 | 0.0 |
| AM - B adipose | | | |
| 94711_Donor 2 | 0.0 | 0.0 | 0.0 |
| AM - C_adipose | | | |
| 94712_Donor 2 | 0.0 | 0.0 | 0.0 |
| AD - A_adipose | | | |
| 94713_Donor 2 | 0.0 | 0.0 | 0.0 |
| AD - B_adipose | | | |
| 94714_Donor 2 | 0.0 | 0.0 | 0.0 |
| AD - C_adipose | | | |
| 94742_Donor 3 | 0.0 | 0.0 | 0.0 |
| U - | | | |
| A_Mesenchymal | | | |
| Stem Cells | | | |
| 94743_Donor 3 | 0.0 | 0.0 | 0.0 |
| U - | | | |
| B_Mesenchymal | | | |
| Stem Cells | | | |
| 94730_Donor 3 | 0.0 | 0.0 | 0.0 |
| AM - A_adipose | | | |
| 94731_Donor 3 | 0.0 | 0.0 | 0.0 |
| AM - B_adipose | | | |
| 94732_Donor 3 | 0.0 | 0.0 | 0.0 |
| AM - C_adipose | | | |
| 94733_Donor 3 | 0.0 | 0.0 | 0.0 |
| AD - A_adipose | | | |
| 94734_Donor 3 | 0.0 | 0.0 | 0.0 |
| AD - B_adipose | | | |
| 94735_Donor 3 | 0.0 | 0.0 | 0.0 |
| AD - C_adipose | | | |
| 77138_Liver_{—} | 0.0 | 0.0 | 0.0 |
| HepG2untreated | | | |
| 73556_Heart_Car- | 0.0 | 0.0 | 0.0 |
| diac stromal cells | | | |
| (primary) | | | |
| 81735_Small | 8.5 | 6.3 | 5.1 |
| Intestine | | | |
| 72409_Kidney_{—} | 0.0 | 0.0 | 0.0 |
| Proximal | | | |
| Convoluted | | | |
| Tubule | | | |
| 82685_Small | 0.0 | 0.0 | 5.4 |
| intestine_Duode- | | | |
| num | | | |
| 90650_Adrenal_{—} | 100.0 | 49.3 | 100.0 |
| Adrenocortical | | | |
| adenoma | | | |
| 72410_Kidney_{—} | 0.0 | 0.0 | 0.0 |
| HRCE | | | |
| 72411_Kidney_{—} | 0.0 | 0.0 | 0.0 |
| HRE | | | |
| 73139_Uterus_{—} | 0.0 | 0.0 | 0.0 |
| Uterine smooth | | | |
| muscle cells | | | |

General_screening_panel_v1.5 Summary: Ag6006 Two experiments with same probe-primer sets are in excellent agreement with highest expression of this gene detected in liver (CTs=26). Interestingly, expression of this gene is higher in adult as compared to fetal liver (CTs=32-33). Therefore, expression of this gene may be useful in distinguishing between adult and fetal liver.

In addition, moderate to low expression of this gene is also detected in tissues with metabolic/endocrine functions including pancreas, adipose, adrenal gland, thyroid, and stomach. This gene codes for Serine dehydratase (SD). SD catalyzes the PLP-dependent alpha, beta-elimination of L-serine to pyruvate and ammonia. It is one of three enzymes that are regarded as metabolic exits of the serine-glycine pool. SD is critical for hepatic glucose production. Therefore, inhibition of SD would decrease gluconeogenesis, thus an antagonist of SD would be beneficial for treatment hyperglycemia and diabetes.

In addition moderate levels of expression of this gene is in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Oncology_cell_line_screening_panel_v3.1 Summary: Ag6006 Two experiments with same probe-primer sets are in excellent agreement, with highest expression of this gene detected in cerebellum (CTs=32-33.7). In addition, low levels of expression of this gene is also detected in histiocytic lymphoma. Therefore, therapeutic modulation of this gene may be useful in the treatment of ataxia, autism and histiocytic lymphoma.

Panel 4.1D Summary: Ag6006 Highest expression of this gene is detected in liver cirrhosis sample (CT=29). In addition, moderate to low expression of this gene resting macrophage, LPS activated monocytes and macrophages, dendritic cells, resting and PMA/ionomycin activated LAK cells and normal tissues represented by thymus and kidney. Therefore, therapeutic modulation of this gene may be useful in the treatment of liver cirrhosis, asthma, emphysema, inflammatory bowel disease, arthritis and psoriasis.

Results from another experiment with this gene (run 225245206) are not included. The amp plot indicates that there were experimental difficulties with this run.

Panel 5 Islet Summary: Ag6006 Three experiments with same probe and primer sets are in good agreement. Low expression of this gene is detected mainly in islet cells and adrenocortical adenoma cells (CTs=33-34.8). Therefore, therapeutic modulation of this gene of SD encoded by this gene through the use of small molecule drug may be useful in the treatment of adrenocortical adenoma and metabolic disorders especially type II diabetes.

O. CG151359-01: Lactate Dehydrogenase A Like.

Expression of gene CG151359-01 was assessed using the primer-probe set Ag5225, described in Table OA. Results of the RTQ-PCR runs are shown in Table OB.

TABLE OA
Probe Name Ag5225

| | | | Start | SEQ ID |
|---|---|---|---|---|
| Primers | Sequences | Length | Position | No |
| Forward | 5′-tgttattggaagcggctgta-3′ | 20 | 618 | 513 |
| Probe | TET-5′ctgttcgttttcaattcttcattgga-3′-TAMRA | 26 | 647 | 514 |
| Reverse | 5′-cagagtggataccaagcttttg-3′ | 22 | 673 | 515 |

TABLE OB
General_screemng_panel_v1.5

| | Rel. Exp. (%) |
|---|---|
| | Ag5225, Run |
| Tissue Name | 228763462 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 7.9 |
| Melanoma* SK-MEL-5 | 0.0 |
| Squamous cell carcinoma SCC-4 | 0.0 |
| Testis Pool | 100.0 |
| Prostate ca.* (bone met) PC-3 | 0.0 |
| Prostate Pool | 10.5 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 3.7 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 0.0 |
| Lung | 0.0 |
| Fetal Lung | 0.0 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 5.8 |
| Liver ca. HepG2 | 0.0 |
| Kidney Pool | 0.3 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.7 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.6 |
| Colon ca. HCT-116 | 0.0 |
| Colon ca. CaCo-2 | 49.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 75.3 |
| Small Intestine Pool | 1.8 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 25.2 |
| CNS cancer (neuro; met) SK-N-AS | 0.0 |
| CNS cancer (astro) SF-539 | 0.0 |
| CNS cancer (astro)SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 8.6 |
| Brain (Thalamus) Pool | 0.0 |
| Brain (whole) | 0.0 |
| Spinal Cord Pool | 0.0 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 0.0 |

CNS_neurodegeneration_v1.0 Summary: Ag5225 Expression of this gene is low/undetectable in all samples on this panel (CTs>35). (Data not shown.)

General_screening_panel_v1.5 Summary: Ag5225 Expression of this gene is limited to a few samples on this panel, with highest expression seen in testis (CT=31.8). Moderate to low levels of expression are also seen in normal colon, a colon cancer cell line, and a brain cancer cell line.

Panel 4.1D Summary: Ag5225 Expression of this gene is low/undetectable in all samples on this panel (CTs>35). (Data not shown.)

Panel 5 Islet Summary: Ag5225 Expression of this gene is low/undetectable in all samples on this panel (CTs>35). (Data not shown.)

P. CG152227-01: 3-Hydroxyisobutyryl-Coenzyme A Hydrolase.

Expression of gene CG152227-01 was assessed using the primer-probe set Ag6857, described in Table PA.

TABLE PA
Probe Name Ag6857

| | | | Start | SEQ ID |
|---|---|---|---|---|
| Primers | Sequences | Length | Position | No |
| Forward | 5′-ttggactctggtcttcaagtat-3′ | 22 | 186 | 516 |
| Probe | TET-5′-agacttgtctcgatcaatcttagactctgtatggtaa-3′-TAMRA | 37 | 211 | 517 |
| Reverse | 5′-cttcaaaagaaaatattgcatctg-3′ | 24 | 258 | 518 |

General_screening_panel_v1.6 Summary: Ag6857 Expression of this gene is low/undetectable in all samples on this panel (CTs>35). (Data not shown.)

Q. CG152547-01: Similar to Zinc Transporter 1.

Expression of gene CG152547-01 was assessed using the primer-probe set Ag7619, described in Table QA.

TABLE QA
Probe Name Ag7619

| | | | Start | SEQ ID |
|---|---|---|---|---|
| Primers | Sequences | Length | Position | No |
| Forward | 5′-tgctcatcttccatcaccaa-3′ | 20 | 392 | 519 |
| Probe | TET-5′-ccctaatctcaagtaatcagggacacaa-3′-TAMRA | 28 | 413 | 520 |
| Reverse | 5′-tggttttcctaggcagagga-3′ | 20 | 462 | 521 |

CNS_neurodegeneration_v1.0 Summary: Ag7619 Expression of this gene is low/undetectable in all samples on this panel (CTs>35). (Data not shown.)

Panel 4.1D Summary: Ag7619 Expression of this gene is low/undetectable in all samples on this panel (CTs>35). (Data not shown.)

R. CG152646-01: Amidase.

Expression of gene CG 152646-01 was assessed using the primer-probe set Ag6876, described in Table RA.

TABLE RA
Probe Name Ag6876

| | | | Start | SEQ ID |
|---|---|---|---|---|
| Primers | Sequences | Length | Position | No |
| Forward | 5′-cacatctgtgaccatattgtt-3′ | 21 | 573 | 522 |
| Probe | TET-5′-tttaactggtccaaatacaccatctgtg-3′-TAMRA | 28 | 613 | 523 |
| Reverse | 5′-tttgctatgggatctg-3′ | 16 | 645 | 524 |

General_screening_panel_v1.6 Summary: Ag6876 Expression of this gene is low/undetectable in all samples on this panel (CTs>35). (Data not shown.)

S. CG152959-01: Prenyl Protein-Specific Endoprotease 2.

Expression of gene CG152959-01 was assessed using the primer-probe set Ag7172, described in Table SA. Results of the RTQ-PCR runs are shown in Table SB. Please note that CG152959-01 represents a full-length physical clone.

TABLE SA
Probe Name Ag7172

| | | | Start | SEQ ID |
|---|---|---|---|---|
| Primers | Sequences | Length | Position | No |
| Forward | 5′-cctggaggacgtgctgt-3′ | 17 | 191 | 525 |
| Probe | TET-5′-ccaacctgtcagagtggctgagtccc-3′-TAMRA | 26 | 223 | 526 |
| Reverse | 5′-gcgcttgcggaagg-3′ | 14 | 273 | 527 |

TABLE SB
General_screening_panel_v1.7

| | Rel. Exp. (%) |
|---|---|
| | Ag7172, Run |
| Tissue Name | 318039790 |
| Adipose | 10.6 |
| HUVEC | 35.8 |
| Melanoma* Hs688(A).T | 0.3 |
| Melanoma* Hs688(B).T | 66.9 |
| Melanoma (met) SK-MEL-5 | 4.4 |
| Testis | 13.5 |
| Prostate ca. (bone met) PC-3 | 0.5 |
| Prostate ca. DU145 | 19.3 |
| Prostate pool | 7.7 |
| Uterus pool | 2.5 |
| Ovarian ca. OVCAR-3 | 14.1 |
| Ovarian ca. (ascites) SK-OV-3 | 0.8 |
| Ovarian ca. OVCAR-4 | 51.4 |
| Ovarian ca. OVCAR-5 | 29.1 |
| Ovarian ca. IGROV-1 | 100.0 |
| Ovarian ca. OVCAR-8 | 24.0 |
| Ovary | 3.2 |
| Breast ca. MCF-7 | 17.7 |
| Breast ca. MDA-MB-231 | 43.8 |
| Breast ca. BT-549 | 14.1 |
| Breast ca. T47D | 15.5 |
| Breast pool | 7.5 |
| Trachea | 15.8 |
| Lung | 1.2 |
| Fetal Lung | 9.0 |
| Lung ca. NCI-N417 | 10.0 |
| Lung ca. LX-1 | 4.4 |
| Lung ca. NCI-H146 | 15.5 |
| Lung ca. SHP-77 | 38.2 |
| Lung ca. NCI-H23 | 26.2 |
| Lung ca. NCI-H460 | 8.5 |
| Lung ca. HOP-62 | 9.6 |
| Lung ca. NCI-H522 | 56.3 |
| Lung ca. DMS-114 | 8.8 |
| Liver | 0.0 |
| Fetal Liver | 1.0 |
| Kidney pool | 32.3 |
| Fetal Kidney | 3.7 |
| Renal ca. 786-0 | 40.1 |
| Renal ca. A498 | 12.7 |
| Renal ca. ACHN | 15.0 |
| Renal ca. UO-31 | 22.8 |
| Renal ca. TK-10 | 46.0 |
| Bladder | 1.6 |
| Gastric ca. (liver met.) NCI-N87 | 0.0 |
| Stomach | 0.0 |
| Colon ca. SW-948 | 6.0 |
| Colon ca. SW480 | 0.4 |
| Colon ca. (SW480 met) SW620 | 6.8 |
| Colon ca. HT29 | 30.4 |
| Colon ca. HCT-116 | 22.2 |
| Colon cancer tissue | 1.0 |
| Colon ca. SW1116 | 6.1 |
| Colon ca. Colo-205 | 11.0 |
| Colon ca. SW-48 | 9.4 |
| Colon | 15.9 |
| Small Intestine | 1.5 |
| Fetal Heart | 0.7 |
| Heart | 1.2 |
| Lymph Node pool | 3.1 |
| Lymph Node pool | 26.1 |
| Fetal Skeletal Muscle | 1.7 |
| Skeletal Muscle pool | 0.3 |
| Skeletal Muscle | 0.2 |
| Spleen | 4.4 |
| Thymus | 14.7 |
| CNS cancer (glio/astro) SF-268 | 6.4 |
| CNS cancer (glio/astro) T98G | 3.3 |
| CNS cancer (neuro; met) SK-N-AS | 0.2 |
| CNS cancer (astro) SF-539 | 8.9 |
| CNS cancer (astro) SNB-75 | 10.1 |
| CNS cancer (glio) SNB-19 | 16.5 |
| CNS cancer (glio) SF-295 | 4.9 |
| Brain (Amygdala) | 6.6 |
| Brain (Cerebellum) | 12.8 |
| Brain (Fetal) | 25.5 |
| Brain (Hippocampus) | 4.7 |
| Cerebral Cortex pool | 1.8 |
| Brain (Substantia nigra) | 4.0 |
| Brain (Thalamus) | 4.3 |
| Brain (Whole) | 21.6 |
| Spinal Cord | 0.8 |
| Adrenal Gland | 2.2 |
| Pituitary Gland | 11.9 |
| Salivary Gland | 8.0 |
| Thyroid | 8.4 |
| Pancreatic ca. PANC-1 | 10.5 |
| Pancreas pool | 1.5 |

General_screening_panel_v1.7 Summary: Ag7172 Highest expression of this gene is detected in ovarian cancer IGROV-1 cell line (CT=28.3). Moderate levels of expression of this gene is also seen in cluster of cancer cell lines derived from pancreatic, gastric, colon, lung, liver, renal, breast, ovarian, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, melanoma and brain cancers.

Among tissues with metabolic or endocrine function, this gene is expressed at moderate to low levels in pancreas, adipose, adrenal gland, thyroid, pituitary gland, fetal skeletal muscle, heart, fetal liver and the gastrointestinal tract. Therefore, therapeutic modulation of the activity of this gene may prove useful in the treatment of endocrine/metabolically related diseases, such as obesity and diabetes.

In addition, this gene is expressed at low levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

T. CG153033-01: NA-Dependent Inorganic Phosphate Cotransporter.

Expression of gene CG153033-01 was assessed using the primer-probe set Ag5798, described in Table TA. Results of the RTQ-PCR runs are shown in Tables TB and TC.

TABLE TA
Probe Name Ag5798

| | | | Start | SEQ ID |
|---|---|---|---|---|
| Primers | Sequences | Length | Position | No |
| Forward | 5′-aatcttggagttgccattgtg-3′ | 21 | 223 | 528 |
| Probe | TET-5′-ccatcaacatatacggtgctattgttgacc-3′-TAMRA | 30 | 249 | 529 |
| Reverse | 5′-tcccagttaaactgtgctgtct-3′ | 22 | 284 | 530 |

TABLE TB
CNS_neurodegeneration_v1.0

| | Rel. Exp. (%) |
|---|---|
| | Ag5798, Run |
| Tissue Name | 247179626 |
| AD 1 Hippo | 8.0 |
| AD 2 Hippo | 14.4 |
| AD 3 Hippo | 3.7 |
| AD 4 Hippo | 7.3 |
| AD 5 hippo | 24.1 |
| AD 6 Hippo | 24.8 |
| Control 2 Hippo | 42.6 |
| Control 4 Hippo | 3.3 |
| Control (Path) 3 Hippo | 0.0 |
| AD 1 Temporal Ctx | 9.3 |
| AD 2 Temporal Ctx | 94.6 |
| AD 3 Temporal Ctx | 3.6 |
| AD 4 Temporal Ctx | 13.6 |
| AD 5 Inf Temporal Ctx | 100.0 |
| AD 5 Sup Temporal Ctx | 71.7 |
| AD 6 Inf Temporal Ctx | 57.8 |
| AD 6 Sup Temporal Ctx | 22.8 |
| Control 1 Temporal Ctx | 0.0 |
| Control 2 Temporal Ctx | 38.7 |
| Control 3 Temporal Ctx | 12.6 |
| Control 4 Temporal Ctx | 10.0 |
| Control (Path) 1 Temporal Ctx | 70.2 |
| Control (Path) 2 Temporal Ctx | 8.2 |
| Control (Path) 3 Temporal Ctx | 0.0 |
| Control (Path) 4 Temporal Ctx | 39.0 |
| AD 1 Occipital Ctx | 0.0 |
| AD 2 Occipital Ctx (Missing) | 0.0 |
| AD 3 Occipital Ctx | 0.0 |
| AD 4 Occipital Ctx | 24.7 |
| AD 5 Occipital Ctx | 9.3 |
| AD 6 Occipital Ctx | 40.6 |
| Control 1 Occipital Ctx | 3.0 |
| Control 2 Occipital Ctx | 21.3 |
| Control 3 Occipital Ctx | 3.5 |
| Control 4 Occipital Ctx | 0.0 |
| Control (Path) 1 Occipital Ctx | 54.0 |
| Control (Path) 2 Occipital Ctx | 0.0 |
| Control (Path) 3 Occipital Ctx | 0.0 |
| Control (Path) 4 Occipital Ctx | 3.4 |
| Control 1 Parietal Ctx | 0.0 |
| Control 2 Parietal Ctx | 59.9 |
| Control 3 Parietal Ctx | 0.0 |
| Control (Path) 1 Parietal Ctx | 46.7 |
| Control (Path) 2 Parietal Ctx | 16.0 |
| Control (Path) 3 Parietal Ctx | 7.8 |
| Control (Path) 4 Parietal Ctx | 17.6 |

TABLE TC
General_screening_panel_v1.5

| | Rel. Exp. (%) |
|---|---|
| | Ag5798, Run |
| Tissue Name | 245274436 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 1.9 |
| Melanoma* SK-MEL-5 | 0.0 |
| Squamous cell carcinoma SCC-4 | 0.9 |
| Testis Pool | 5.9 |
| Prostate ca.* (bone met) PC-3 | 0.4 |
| Prostate Pool | 0.0 |
| Placenta | 3.9 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 1.0 |
| Ovarian ca. OVCAR-8 | 0.0 |
| Ovary | 3.1 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 1.6 |
| Trachea | 0.0 |
| Lung | 0.0 |
| Fetal Lung | 27.9 |
| Lung ca. NCI-N417 | 0.0 |
| Lung ca. LX-1 | 1.2 |
| Lung ca. NCI-H146 | 0.0 |
| Lung ca. SHP-77 | 0.0 |
| Lung ca. A549 | 0.8 |
| Lung ca. NCI-H526 | 90.8 |
| Lung ca. NCI-H23 | 0.6 |
| Lung ca. NCI-H460 | 0.0 |
| Lung ca. HOP-62 | 0.0 |
| Lung ca. NCI-H522 | 0.0 |
| Liver | 1.6 |
| Fetal Liver | 51.1 |
| Liver ca. HepG2 | 0.0 |
| Kidney Pool | 0.0 |
| Fetal Kidney | 5.1 |
| Renal ca. 786-0 | 0.0 |
| Renal ca. A498 | 0.0 |
| Renal ca. ACHN | 1.1 |
| Renal ca. UO-31 | 0.0 |
| Renal ca. TK-10 | 0.0 |
| Bladder | 1.5 |
| Gastric ca. (liver met.) NCI-N87 | 1.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.9 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 2.1 |
| Stomach Pool | 1.3 |
| Bone Marrow Pool | 1.6 |
| Fetal Heart | 5.9 |
| Heart Pool | 0.0 |
| Lymph Node Pool | 0.0 |
| Fetal Skeletal Muscle | 0.0 |
| Skeletal Muscle Pool | 1.1 |
| Spleen Pool | 0.9 |
| Thymus Pool | 7.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 14.7 |
| Brain (cerebellum) | 3.4 |
| Brain (fetal) | 20.2 |
| Brain (Hippocampus) Pool | 45.4 |
| Cerebral Cortex Pool | 19.6 |
| Brain (Substantia nigra) Pool | 29.7 |
| Brain (Thalamus) Pool | 100.0 |
| Brain (whole) | 10.4 |
| Spinal Cord Pool | 6.1 |
| Adrenal Gland | 0.0 |
| Pituitary gland Pool | 5.0 |
| Salivary Gland | 0.0 |
| Thyroid (female) | 0.0 |
| Pancreatic ca. CAPAN2 | 0.0 |
| Pancreas Pool | 5.7 |

CNS_neurodegeneration_v1.0 Summary: Ag5798 This panel does not show differential expression of this gene in Alzheimer's disease. However, this profile confirms the expression of this gene at moderate levels in the brain. Please see Panel 1.5 for discussion of utility of this gene in the central nervous system.

General_screening_panel_v1.5 Summary: Ag5798 Highest expression of this gene is seen in the thalamus (CT=31.3). This gene is also expressed at low to significant levels in the amygdala, hippocampus, cerebral cortex, substantia nigra, and whole and fetal brain samples. Therefore, therapeutic modulation of the expression or function of this gene may be useful in the treatment of neurological disorders, such as Alzheimer's disease, Parkinson's disease, schizophrenia, multiple sclerosis, stroke and epilepsy.

In addition, this gene is expressed at much higher levels in fetal liver tissue (CT=32.5) when compared to expression in the adult counterpart (CT=37). Thus, expression of this gene may be used to differentiate between the fetal and adult source of this tissue.

Moderate expression is also seen in a single lung cancer cell line (CT=31). Thus, expression of this gene could be used as a marker to detect the presence of lung cancer. Furthermore, therapeutic modulation of the expression or function of this gene may be effective in the treatment of lung cancer.

Panel 4.1D Summary: Ag5798 Expression of this gene is low/undetectable in all samples on this panel (CTs>35). (Data not shown.)

Panel 5 Islet Summary: Ag5798 Expression of this gene is low/undetectable in all samples on this panel (CTs>35). (Data not shown.)

U. CG153818-01: Kinesin 19A.

Expression of gene CG153818-01 was assessed using the primer-probe set Ag5692, described in Table UA. Results of the RTQ-PCR runs are shown in Tables UB, UC and UD.

TABLE UA
Probe Name Ag5692

| | | | Start | SEQ ID |
|---|---|---|---|---|
| Primers | Sequences | Length | Position | No |
| Forward | 5′-cgacaagggtagcaacaagtac-3′ | 22 | 1149 | 531 |
| Probe | TET-5′-atcaactatcgcgacagcaagctcac-3′-TAMRA | 26 | 1171 | 532 |
| Reverse | 5′-gtttcctcccagagagtcctt-3′ | 21 | 1207 | 533 |

TABLE UB
CNS_neurodegeneration_v1.0

| | Rel. Exp. (%) | Rel. Exp. (%) |
|---|---|---|
| Tissue | Ag5692, Run | Ag5692, Run |
| Name | 247018768 | 264979292 |
| AD 1 Hippo | 5.1 | 5.3 |
| AD 2 Hippo | 23.3 | 26.6 |
| AD 3 Hippo | 4.1 | 5.2 |
| AD 4 Hippo | 19.1 | 22.8 |
| AD 5 Hippo | 28.9 | 39.8 |
| AD 6 Hippo | 74.7 | 88.3 |
| Control 2 Hippo | 19.8 | 27.0 |
| Control 4 Hippo | 10.7 | 11.6 |
| Control (Path) 3 Hippo | 6.9 | 7.9 |
| AD 1 Temporal Ctx | 10.4 | 17.2 |
| AD 2 Temporal Ctx | 18.0 | 17.6 |
| AD 3 Temporal Ctx | 2.7 | 8.5 |
| AD 4 Temporal Ctx | 29.1 | 33.4 |
| AD 5 Inf Temporal Ctx | 100.0 | 100.0 |
| AD 5 Sup Temporal Ctx | 66.4 | 67.8 |
| AD 6 Inf Temporal Ctx | 94.6 | 93.3 |
| AD 6 Sup Temporal Ctx | 59.0 | 72.2 |
| Control 1 Temporal Ctx | 2.2 | 2.6 |
| Control 2 Temporal Ctx | 17.9 | 21.8 |
| Control 3 Temporal Ctx | 4.9 | 6.3 |
| Control 3 Temporal Ctx | 8.9 | 9.0 |
| Control (Path) 1 Temporal Ctx | 8.0 | 11.1 |
| Control (Path) 2 Temporal Ctx | 7.3 | 6.5 |
| Control (Path) 3 Temporal Ctx | 5.6 | 6.9 |
| Control (Path) 4 Temporal Ctx | 5.8 | 4.9 |
| AD 1 Occipital Ctx | 2.9 | 6.2 |
| AD 2 Occipital Ctx (Missing) | 0.0 | 0.0 |
| AD 3 Occipital Ctx | 5.4 | 5.9 |
| AD 4 Occipital Ctx | 33.9 | 30.4 |
| AD 5 Occipital Ctx | 9.5 | 14.2 |
| AD 6 Occipital Ctx | 13.3 | 14.9 |
| Control 1 Occipital Ctx | 2.4 | 2.8 |
| Control 2 Occipital Ctx | 27.2 | 21.5 |
| Control 3 Occipital Ctx | 8.2 | 8.2 |
| Control 4 Occipital Ctx | 9.7 | 12.9 |
| Control (Path) 1 Occipital Ctx | 17.0 | 0.0 |
| Control (Path) 2 Occipital Ctx | 3.7 | 5.8 |
| Control (Path) 3 Occipital Ctx | 5.8 | 5.8 |
| Control (Path) 4 Occipital Ctx | 3.6 | 5.0 |
| Control 1 Parietal Ctx | 3.8 | 5.2 |
| Control 2 Parietal Ctx | 68.8 | 90.8 |
| Control 3 Parietal Ctx | 6.0 | 9.7 |
| Control (Path) 1 Parietal Ctx | 10.2 | 8.2 |
| Control (Path) 2 Parietal Ctx | 7.5 | 6.8 |
| Control (Path) 3 Parietal Ctx | 3.8 | 5.4 |
| Control (Path) 4 Parietal Ctx | 6.8 | 6.4 |

TABLE UC
General_screening_panel_v1.5

| | Rel. Exp. (%) |
|---|---|
| | Ag5692, Run |
| Tissue Name | 245274428 |
| Adipose | 2.6 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.3 |
| Squamous cell carcinoma SCC-4 | 0.0 |
| Testis Pool | 7.1 |
| Prostate ca.* (bone met) PC-3 | 0.0 |
| Prostate Pool | 0.6 |
| Placenta | 0.0 |
| Uterus Pool | 1.8 |
| Ovarian ca. OVCAR-3 | 1.3 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.7 |
| Ovarian ca. OVCAR-8 | 0.0 |
| Ovary | 2.3 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.9 |
| Trachea | 51.1 |
| Lung | 0.6 |
| Fetal Lung | 52.9 |
| Lung ca. NCI-N417 | 0.0 |
| Lung ca. LX-1 | 15.2 |
| Lung ca. NCI-H146 | 0.0 |
| Lung ca. SHP-77 | 100.0 |
| Lung ca. A549 | 0.0 |
| Lung ca. NCI-H526 | 0.4 |
| Lung ca. NCI-H23 | 2.9 |
| Lung ca. NCI-H460 | 0.0 |
| Lung ca. HOP-62 | 0.0 |
| Lung ca. NCI-H522 | 0.0 |
| Liver | 2.6 |
| Fetal Liver | 2.5 |
| Liver ca. HepG2 | 0.0 |
| Kidney Pool | 1.9 |
| Fetal Kidney | 1.6 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 14.0 |
| Gastric ca. (liver met.) NCI-N87 | 0.0 |
| Gastric ca. KATO III | 0.0 |
| Colon ca. SW-948 | 0.6 |
| Colon ca. SW480 | 0.4 |
| Colon ca.* (SW480 met) SW620 | 4.8 |
| Colon ca. HT29 | 2.0 |
| Colon ca. HCT-116 | 0.4 |
| Colon ca. CaCo-2 | 0.4 |
| Colon cancer tissue | 1.9 |
| Colon ca. SW1116 | 0.0 |
| Colon ca. Colo-205 | 0.0 |
| Colon ca. SW-48 | 0.7 |
| Colon Pool | 0.3 |
| Small Intestine Pool | 3.2 |
| Stomach Pool | 3.6 |
| Bone Marrow Pool | 2.0 |
| Fetal Heart | 0.3 |
| Heart Pool | 0.5 |
| Lymph Node Pool | 0.9 |
| Fetal Skeletal Muscle | 2.7 |
| Skeletal Muscle Pool | 0.7 |
| Spleen Pool | 54.7 |
| Thymus Pool | 9.9 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 1.0 |
| CNS cancer (glio) SF-295 | 0.6 |
| Brain (Amygdala) Pool | 27.2 |
| Brain (cerebellum) | 8.2 |
| Brain (fetal) | 3.1 |
| Brain (Hippocampus) Pool | 26.2 |
| Cerebral Cortex Pool | 15.9 |
| Brain (Substantia nigra) Pool | 15.4 |
| Brain (Thalamus) Pool | 35.1 |
| Brain (whole) | 11.3 |
| Spinal Cord Pool | 16.2 |
| Adrenal Gland | 1.2 |
| Pituitary gland Pool | 0.2 |
| Salivary Gland | 3.9 |
| Thyroid (female) | 15.8 |
| Pancreatic ca. CAPAN2 | 0.0 |
| Pancreas Pool | 4.4 |

TABLE UD
Panel 4.1D

| | Rel. Exp. (%) |
|---|---|
| | Ag5692, Run |
| Tissue Name | 246504798 |
| Secondary Th1 act | 0.0 |
| Secondary Th2 act | 1.4 |
| Secondary Tr1 act | 0.0 |
| Secondary Th1 rest | 0.8 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 1.4 |
| 2ry Th1/Th2/Tr1_anti-CD95 CH11 | 0.0 |
| LAK cells rest | 0.0 |
| LAK cells IL-2 | 3.2 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 29.7 |
| Two Way MLR 3 day | 0.0 |
| Two Way MLR 5 day | 0.0 |
| Two Way MLR 7 day | 0.0 |
| PBMC rest | 2.3 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 2.0 |
| HUVEC IFN gamma | 100.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 7.0 |
| KU-812 (Basophil) PMA/ionomycin | 11.0 |
| CCD1106 (Keratinocytes) none | 0.0 |
| CCD1106 (Keratinocytes) TNFalpha + IL-1beta | 2.3 |
| Liver cirrhosis | 5.1 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 1.2 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 1.3 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 0.0 |
| Lung | 3.6 |
| Thymus | 1.3 |
| Kidney | 0.0 |

CNS_neurodegeneration_v1.0 Summary: Ag5692 Two experiments with the same probe and primer set produce results that are in excellent agreement. This panel confirms the expression of this gene at moderate levels in the brain in an independent group of individuals. This gene is found to be upregulated in the temporal cortex of Alzheimer's disease patients. This gene encodes a putative kinesin, a microtubule-based motor protein involved in the transport of organelles. Axonal transport of APP in neurons is mediated by binding with kinesin. (Gunewardena S, Neuron Nov. 8, 2001;32(3):389-401). Kamal et al. suggest that impaired APP transport leads to enhanced axonal generation and deposition of Abeta, resulting in disruption of neurotrophic signaling and neurodegeneration (Nature Dec. 6, 2001;414(6864):643-8). Thus, therapeutic modulation of the expression or function of this gene may be useful in the treatment of neurodegenerative disorders, and specifically may decrease neuronal death and be of use in the treatment of Alzheimer's disease.

General_screening_panel_v1.5 Summary: Ag5692 Highest expression of this gene is seen in a lung cancer cell line (CT=29.4). Moderate levels of expression are also seen in fetal lung (CT=30) and interestingly, are much higher than expression of this gene in the adult counterpart (CT=32). Thus, expression of this gene could be used to differentiate between the adult and fetal source of this tissue. In addition, therapeutic modulation of the expression or function of this gene may be useful in the treatment of diseases that affect the lung, including lung cancer.

Moderate to low levels of expression are seen in all regions of the CNS examined. Please see CNS_neurodegeneration_v1.0 for discussion of utility of this gene in CNS disorders.

Low but significant levels of expression are also seen in pancreas, thyroid, fetal skeletal muscle, adipose and adult and fetal liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

Panel 4.1D Summary: Ag5692 Expression of this gene is limited to a few samples in this panel, with highest expression in IFN-gamma treated HUVEC cells (CT=31.2). Low but significant levels of expression are also seen in PMA/ionomycin treated basophils and resting NK cells. This expression profile suggests that expression of this gene could be a marker of activated HUVEC cells. In addition, modulation of the expression or function of this gene product may reduce or eliminate the symptoms in patients with autoimmune and inflammatory diseases that involve endothelial cells, such as lupus erythematosus, asthma, emphysema, Crohn's disease, ulcerative colitis, rheumatoid arthritis, osteoarthritis, and psoriasis.

V. CG154435-01: Dynein Beta Chain, Ciliary.

Expression of gene CG154435-01 was assessed using the primer-probe set Ag5694, described in Table VA. Results of the RTQ-PCR runs are shown in Tables VB, VC, VD, VE and VF.

TABLE VA
Probe Name Ag5694

| | | | Start | SEQ ID |
|---|---|---|---|---|
| Primers | Sequence | Length | Position | No |
| Forward | 5′-ccaccaagtggaaagatatcaa | 22 | 3932 | 534 |
| Probe | TET-5′-ccttggcaaaacttcttacaatctatgtcca-3′-TAMRA | 30 | 3965 | 535 |
| Reverse | 5′-ccttgtccaaagacctcatgt-3′ | 21 | 3995 | 536 |

TABLE VB
AI_comprehensive panel_v1.0

| | Rel. Exp. (%) |
|---|---|
| | Ag5694, Run |
| Tissue Name | 245243118 |
| 110967 COPD-F | 0.3 |
| 110980 COPD-F | 0.0 |
| 110968 COPD-M | 0.2 |
| 110977 COPD-M | 0.0 |
| 110989 Emphysema-F | 0.1 |
| 110992 Emphysema-F | 0.0 |
| 110993 Emphysema-F | 0.0 |
| 110994 Emphysema-F | 0.0 |
| 110995 Emphysema-F | 0.4 |
| 110996 Emphysema-F | 0.7 |
| 110997 Asthma-M | 0.3 |
| 111001 Asthma-F | 0.0 |
| 111002 Asthma-F | 0.0 |
| 111003 Atopic Asthma-F | 0.0 |
| 111004 Atopic Asthma-F | 0.1 |
| 111005 Atopic Asthma-F | 0.0 |
| 111006 Atopic Asthma-F | 0.0 |
| 111417 Allergy-M | 1.0 |
| 112347 Allergy-M | 0.0 |
| 112349 Normal Lung-F | 0.5 |
| 112357 Normal Lung-F | 0.0 |
| 112354 Normal Lung-M | 9.7 |
| 112374 Crohns-F | 0.0 |
| 112389 Match Control Crohns-F | 0.2 |
| 112375 Crohns-F | 0.5 |
| 112732 Match Control Crohns-F | 0.2 |
| 112725 Crohns-M | 0.0 |
| 112387 Match Control Crohns-M | 0.0 |
| 112378 Crohns-M | 3.6 |
| 112390 Match Control Crohns-M | 0.0 |
| 112726 Crohns-M | 0.0 |
| 112731 Match Control Crohns-M | 0.2 |
| 112380 Ulcer Col-F | 0.0 |
| 112734 Match Control Ulcer Col-F | 0.5 |
| 112384 Ulcer Col-F | 0.0 |
| 112737 Match Control Ulcer Col-F | 0.0 |
| 112386 Ulcer Col-F | 100.0 |
| 112738 Match Control Ulcer Col-F | 3.0 |
| 112381 Ulcer Col-M | 0.2 |
| 112735 Match Control Ulcer Col-M | 2.2 |
| 112382 Ulcer Col-M | 0.2 |
| 112394 Match Control Ulcer Col-M | 0.0 |
| 112383 Ulcer Col-M | 0.3 |
| 112736 Match Control Ulcer Col-M | 0.1 |
| 112423 Psoriasis-F | 0.4 |
| 112427 Match Control Psoriasis-F | 0.0 |
| 112418 Psoriasis-M | 6.8 |
| 112723 Match Control Psoriasis-M | 2.6 |
| 112419 Psoriasis-M | 2.7 |
| 112424 Match Control Psoriasis-M | 2.9 |
| 112420 Psoriasis-M | 0.6 |
| 112425 Match Control Psoriasis-M | 2.3 |
| 104689 (MF) OA Bone-Backus | 0.2 |
| 104690 (MF) Adj “Normal” Bone-Backus | 2.6 |
| 104691 (MF) OA Synovium-Backus | 0.7 |
| 104692 (BA) OA Cartilage-Backus | 2.0 |
| 104694 (BA) OA Bone-Backus | 0.3 |
| 104695 (BA) Adj “Normal” Bone-Backus | 0.4 |
| 104696 (BA) OA Synovium-Backus | 0.0 |
| 104700 (SS) OA Bone-Backus | 0.0 |
| 104701 (SS) Adj “Normal” Bone-Backus | 1.5 |
| 104702 (SS) OA Synovium-Backus | 2.6 |
| 117093 OA Cartilage Rep7 | 0.2 |
| 112672 OA Bone5 | 0.1 |
| 112673 OA Synovium5 | 2.7 |
| 112674 OA Synovial Fluid cells5 | 0.2 |
| 117100 OA Cartilage Rep14 | 3.1 |
| 112756 OA Bone9 | 1.6 |
| 112757 OA Synovium9 | 0.0 |
| 112758 OA Synovial Fluid Cells9 | 0.4 |
| 117125 RA Cartilage Rep2 | 1.5 |
| 113492 Bone2 RA | 0.0 |
| 113493 Synovium2 RA | 0.9 |
| 113494 Syn Fluid Cells RA | 0.9 |
| 113499 Cartilage4 RA | 51.4 |
| 113500 Bone4 RA | 82.4 |
| 113501 Synovium4 RA | 13.1 |
| 113502 Syn Fluid Cells4 RA | 0.0 |
| 113495 Cartilage3 RA | 14.3 |
| 113496 Bone3 RA | 3.1 |
| 113497 Synovium3 RA | 0.3 |
| 113498 Syn Fluid Cells3 RA | 0.6 |
| 117106 Normal Cartilage Rep20 | 42.3 |
| 113663 Bone3 Normal | 0.4 |
| 113664 Synovium3 Normal | 0.4 |
| 113665 Syn Fluid Cells3 Normal | 0.2 |
| 117107 Normal Cartilage Rep22 | 7.9 |
| 113667 Bone4 Normal | 0.0 |
| 113668 Synovium4 Normal | 0.0 |
| 113669 Syn Fluid Cells4 Normal | 0.0 |

TABLE VC
CNS_neurodegeneration_v1.0

| | Rel. Exp. (%) |
|---|---|
| | Ag5694, Run |
| Tissue Name | 247018769 |
| AD 1 Hippo | 0.0 |
| AD 2 Hippo | 11.4 |
| AD 3 Hippo | 0.0 |
| AD 4 Hippo | 4.5 |
| AD 5 hippo | 0.0 |
| AD 6 Hippo | 33.0 |
| Control 2 Hippo | 0.0 |
| Control 4 Hippo | 0.0 |
| Control (Path) 3 Hippo | 0.0 |
| AD 1 Temporal Ctx | 7.2 |
| AD 2 Temporal Ctx | 17.3 |
| AD 3 Temporal Ctx | 7.1 |
| AD 4 Temporal Ctx | 0.0 |
| AD 5 Inf Temporal Ctx | 7.4 |
| AD 5 Sup Temporal Ctx | 6.4 |
| AD 6 Inf Temporal Ctx | 19.6 |
| AD 6 Sup Temporal Ctx | 100.0 |
| Control 1 Temporal Ctx | 0.0 |
| Control 2 Temporal Ctx | 0.0 |
| Control 3 Temporal Ctx | 0.0 |
| Control 4 Temporal Ctx | 21.0 |
| Control (Path) 1 Temporal Ctx | 6.4 |
| Control (Path) 2 Temporal Ctx | 13.0 |
| Control (Path) 3 Temporal Ctx | 0.0 |
| Control (Path) 4 Temporal Ctx | 15.7 |
| AD 1 Occipital Ctx | 0.0 |
| AD 2 Occipital Ctx (Missing) | 0.0 |
| AD 3 Occipital Ctx | 4.3 |
| AD 4 Occipital Ctx | 7.1 |
| AD 5 Occipital Ctx | 0.0 |
| AD 6 Occipital Ctx | 25.5 |
| Control 1 Occipital Ctx | 0.0 |
| Control 2 Occipital Ctx | 30.6 |
| Control 3 Occipital Ctx | 6.4 |
| Control 4 Occipital Ctx | 5.1 |
| Control (Path) 1 Occipital Ctx | 6.4 |
| Control (Path) 2 Occipital Ctx | 0.0 |
| Control (Path) 3 Occipital Ctx | 0.0 |
| Control (Path) 4 Occipital Ctx | 13.1 |
| Control 1 Parietal Ctx | 0.0 |
| Control 2 Parietal Ctx | 5.0 |
| Control 3 Parietal Ctx | 5.7 |
| Control (Path) 1 Parietal Ctx | 7.7 |
| Control (Path) 2 Parietal Ctx | 13.6 |
| Control (Path) 3 Parietal Ctx | 4.1 |
| Control (Path) 4 Parietal Ctx | 2.1 |

TABLE VD
General_screening_panel_v1.5

| | Rel. Exp. (%) |
|---|---|
| | Ag5694, Run |
| Tissue Name | 249040574 |
| Adipose | 0.6 |
| Melanoma* Hs688(A).T | 0.0 |
| Melanoma* Hs688(B).T | 0.0 |
| Melanoma* M14 | 0.0 |
| Melanoma* LOXIMVI | 0.9 |
| Melanoma* SK-MEL-5 | 11.0 |
| Squamous cell carcinoma SCC-4 | 2.3 |
| Testis Pool | 100.0 |
| Prostate ca.* (bone met) PC-3 | 0.0 |
| Prostate Pool | 0.0 |
| Placenta | 4.3 |
| Uterus Pool | 0.4 |
| Ovarian ca. OVCAR-3 | 5.2 |
| Ovarian ca. SK-OV-3 | 3.3 |
| Ovarian ca. OVCAR-4 | 2.3 |
| Ovarian ca. OVCAR-5 | 1.4 |
| Ovarian ca. IGROV-1 | 1.2 |
| Ovarian ca. OVCAR-8 | 1.6 |
| Ovary | 0.0 |
| Breast ca. MCF-7 | 0.9 |
| Breast ca. MDA-MB-231 | 0.5 |
| Breast ca. BT 549 | 0.0 |
| Breast ca. T47D | 0.0 |
| Breast ca. MDA-N | 0.0 |
| Breast Pool | 0.8 |
| Trachea | 2.6 |
| Lung | 0.0 |
| Fetal Lung | 12.7 |
| Lung ca. NCI-N417 | 0.0 |
| Lung ca. LX-1 | 13.5 |
| Lung ca. NCI-H146 | 0.5 |
| Lung ca. SHP-77 | 8.6 |
| Lung ca. A549 | 1.2 |
| Lung ca. NCI-H526 | 0.0 |
| Lung ca. NCI-H23 | 41.8 |
| Lung ca. NCI-H460 | 0.6 |
| Lung ca. HOP-62 | 0.6 |
| Lung ca. NCI-H522 | 0.0 |
| Liver | 0.0 |
| Fetal Liver | 0.5 |
| Liver ca. HepG2 | 90.1 |
| Kidney Pool | 0.0 |
| Fetal Kidney | 0.9 |
| Renal ca. 786-0 | 0.6 |
| Renal ca. A498 | 1.0 |
| Renal ca. ACHN | 0.7 |
| Renal ca. UO-31 | 1.3 |
| Renal ca. TK-10 | 40.3 |
| Bladder | 1.4 |
| Gastric ca. (liver met.) NCI-N87 | 2.8 |
| Gastric ca. KATO III | 1.1 |
| Colon ca. SW-948 | 0.0 |
| Colon ca. SW480 | 1.6 |
| Colon ca.* (SW480 met) SW620 | 0.6 |
| Colon ca. HT29 | 0.0 |
| Colon ca. HCT-116 | 2.8 |
| Colon ca. CaCo-2 | 0.0 |
| Colon cancer tissue | 2.5 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.3 |
| Spleen Pool | 0.5 |
| Thymus Pool | 3.0 |
| CNS cancer (glio/astro) U87-MG | 0.9 |
| CNS cancer (glio/astro) U-118-MG | 0.3 |
| CNS cancer (neuro; met) SK-N-AS | 0.9 |
| CNS cancer (astro) SF-539 | 0.0 |
| CNS cancer (astro) SNB-75 | 0.3 |
| CNS cancer (glio) SNB-19 | 0.9 |
| CNS cancer (glio) SF-295 | 0.0 |
| Brain (Amygdala) Pool | 0.3 |
| Brain (cerebellum) | 0.5 |
| Brain (fetal) | 0.0 |
| Brain (Hippocampus) Pool | 0.2 |
| Cerebral Cortex Pool | 0.9 |
| Brain (Substantia nigra) Pool | 0.8 |
| Brain (Thalamus) Pool | 0.9 |
| Brain (whole) | 0.0 |
| Spinal Cord Pool | 0.3 |
| Adrenal Gland | 0.2 |
| Pituitary gland Pool | 0.0 |
| Salivary Gland | 0.2 |
| Thyroid (female) | 0.6 |
| Pancreatic ca. CAPAN2 | 6.7 |
| Pancreas Pool | 0.0 |

TABLE VE
Panel 4.1D

| | Rel. Exp. (%) |
|---|---|
| | Ag5694, Run |
| Tissue Name | 246504805 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.4 |
| CD8 lymphocyte act | 0.0 |
| Secondary CD8 lymphocyte rest | 0.9 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.4 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 5.8 |
| NK Cells IL-2 rest | 0.4 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.4 |
| EOL-1 dbcAMP | 0.0 |
| EOL-1 dbcAMP PMA/ionomycin | 0.9 |
| Dendritic cells none | 0.0 |
| Dendritic cells LPS | 0.0 |
| Dendritic cells anti-CD40 | 0.0 |
| Monocytes rest | 0.0 |
| Monocytes LPS | 100.0 |
| Macrophages rest | 0.0 |
| Macrophages LPS | 0.6 |
| HUVEC none | 0.0 |
| HUVEC starved | 0.0 |
| HUVEC IL-1beta | 0.5 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 1.1 |
| CCD1106 (Keratinocytes) TNFalpha + IL-1beta | 1.8 |
| Liver cirrhosis | 0.6 |
| NCI-H292 none | 2.1 |
| NCI-H292 IL-4 | 1.4 |
| NCI-H292 IL-9 | 0.0 |
| NCI-H292 IL-13 | 0.0 |
| NCI-H292 IFN gamma | 0.4 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 13.1 |
| Neutrophils rest | 0.4 |
| Colon | 0.0 |
| Lung | 0.0 |
| Thymus | 0.0 |
| Kidney | 2.4 |

TABLE VF
Panel 5 Islet

| | Rel. Exp. (%) |
|---|---|
| | Ag5694, Run |
| Tissue Name | 253330720 |
| 97457_Patient-02go_adipose | 0.0 |
| 97476_Patient-07sk_skeletal muscle | 0.0 |
| 97477_Patient-07ut_uterus | 0.0 |
| 97478_Patient-07pl_placenta | 0.0 |
| 99167_Bayer Patient 1 | 67.8 |
| 97482_Patient-08ut_uterus | 0.0 |
| 97483_Patient-08pl_placenta | 12.2 |
| 97486_Patient-09sk_skeletal muscle | 5.5 |
| 97487_Patient-09ut_uterus | 0.0 |
| 97488_Patient-09pl_placenta | 7.4 |
| 97492_Patient-10ut_uterus | 0.0 |
| 97493_Patient-10pl_placenta | 0.0 |
| 97495_Patient-11go_adipose | 0.0 |
| 97496_Patient-11sk_skeletal muscle | 0.0 |
| 97497 Patient-11ut_uterus | 0.0 |
| 97498_Patient-11pl_placenta | 16.7 |
| 97500_Patient-12go_adipose | 0.0 |
| 97501_Patient-12sk_skeletal muscle | 0.0 |
| 97502_Patient-12ut_uterus | 0.0 |
| 97503_Patient-12pl_placenta | 8.4 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 7.6 |
| 94735_Donor 3 AD - C_adipose | 0.0 |
| 77138_Liver_HepG2untreated | 100.0 |
| 73556_Heart_Cardiac stromal cells (primary) | 0.0 |
| 81735_Small Intestine | 0.0 |
| 72409_Kidney_Proximal Convoluted Tubule | 0.0 |
| 82685_Small intestine_Duodenum | 0.0 |
| 90650_Adrenal_Adrenocortical adenoma | 0.0 |
| 72410_Kidney_HRCE | 7.6 |
| 72411_Kidney_HRE | 0.0 |
| 73139_Uterus_Uterine smooth muscle cells | 0.0 |

AI_comprehensive panel_v1.0 Summary: Ag5694 Highest expression of this gene is detected in ulcerative colitis sample (CT=30.2). Interestingly, expression of this gene is higher in ulcerative colitis sample as compared to matching control sample (CT=35). Therefore, expression of this gene may be used to distinguish between these two samples and also as a marker to detect ulcerative colitis. In addition, moderate expression of this gene is seen in cartilage, bone and synovium from rheumatoid arthritis patient, low expression in normal lung, psoriasis, and normal cartilage Rep22. Therefore, therapeutic modulation of this gene may be useful in the treatment of rheumatoid arthritis, ulcerative colitis, and psoriasis.

CNS_neurodegeneration_v1.0 Summary: Ag5694 Low expression of this gene is detected in temporal cortex of an Alzheimer's patient. Therefore, therapeutic modulation of this gene may be useful in the treatment of Alzheimer's disease.

General_screening_panel_v1.5 Summary: Ag5694 Highest expression of this gene is detected in testis (CT=29.8). Therefore, expression of this gene may be used to differentiate testis from other samples in this panel. In addition, therapeutic modulation of this gene may be useful in the treatment of testis related diseases including fertility and hypogonadism. In addition, moderate to low levels of expression of this gene is detected in number of cancer cell lines derived from melanoma, pancreatic, renal, liver, lung, and ovarian cancers. Therefore, expression of this gene may be used as diagnostic marker to detect these cancers and also, therapeutic modulation of this gene through the use of antibodies or small molecule drug may be useful in the treatment of melanoma, pancreatic, renal, liver, lung, and ovarian cancers.

Panel 4.1D Summary: Ag5694 Moderate expression of this gene is detected mainly in LPS treated monocytes (CT=29.9). In addition, low levels of expression of this gene is also seen in TNF alpha and LPS treated neutrophils. Therefore, expression of this gene may be used to distinguish activated monocytes and neutrophils from other samples in this panel. The expression of this gene in LPS treated monocytes, cells that play a crucial role in linking innate immunity to adaptive immunity, suggests a role for this gene product in initiating inflammatory reactions. Therefore, modulation of the expression or activity of this gene through the application of monoclonal antibodies may reduce or prevent early stages of inflammation and reduce the severity of inflammatory diseases such as psoriasis, asthma, inflammatory bowel disease, rheumatoid arthritis, osteoarthritis and other lung inflammatory diseases. In addition, small molecule or antibody antagonists of this gene product may be effective in increasing the immune response in patients with AIDS or other immunodeficiencies.

Panel 5 Islet Summary: Ag5694 Low levels of expression of this gene is exclusively seen in liver cancer HepG2 cell line (CT=34.7). Please see panel 1.5 for further utility of this gene.

W. CG154465-01: Kinesin 18B.

Expression of gene CG 154465-01 was assessed using the primer-probe set Ag5695, described in Table WA. Results of the RTQ-PCR runs are shown in Tables WB and WC.

TABLE WA
Probe Name Ag5695

| | | | Start | SEQ ID |
|---|---|---|---|---|
| Primers | Sequence | Length | Position | No |
| Forward | 5′-tcaatgccacctttgatctct-3′ | 21 | 2279 | 537 |
| Probe | TET-5′-aaagcccagtttccatgaatgcattg-3′-TAMRA | 26 | 2316 | 538 |
| Reverse | 5′-cagctcctggggtattttgt-3′ | 20 | 2348 | 539 |

TABLE WB
General_screening_panel_v1.5

| | Rel. Exp. (%) |
|---|---|
| | Ag5695, Run |
| Tissue Name | 245274429 |
| Adipose | 0.1 |
| Melanoma* Hs688(A).T | 0.5 |
| Melanoma* Hs688(B).T | 1.2 |
| Melanoma* M14 | 43.2 |
| Melanoma* LOXIMVI | 45.7 |
| Melanoma* SK-MEL-5 | 17.3 |
| Squamous cell carcinoma SCC-4 | 14.6 |
| Testis Pool | 1.0 |
| Prostate ca.* (bone met) PC-3 | 2.2 |
| Prostate Pool | 0.3 |
| Placenta | 1.5 |
| Uterus Pool | 0.3 |
| Ovarian ca. OVCAR-3 | 39.5 |
| Ovarian ca. SK-OV-3 | 82.4 |
| Ovarian ca. OVCAR-4 | 23.7 |
| Ovarian ca. OVCAR-5 | 33.0 |
| Ovarian ca. IGROV-1 | 9.3 |
| Ovarian ca. OVCAR-8 | 10.5 |
| Ovary | 0.0 |
| Breast ca. MCF-7 | 20.9 |
| Breast ca. MDA-MB-231 | 69.7 |
| Breast ca. BT 549 | 50.0 |
| Breast ca. T47D | 24.1 |
| Breast ca. MDA-N | 24.3 |
| Breast Pool | 0.6 |
| Trachea | 0.6 |
| Lung | 0.1 |
| Fetal Lung | 7.2 |
| Lung ca. NCI-N417 | 13.9 |
| Lung ca. LX-1 | 25.3 |
| Lung ca. NCI-H146 | 14.5 |
| Lung ca. SHP-77 | 25.5 |
| Lung ca. A549 | 55.9 |
| Lung ca. NCI-H526 | 14.9 |
| Lung ca. NCI-H23 | 22.4 |
| Lung ca. NCI-H460 | 0.0 |
| Lung ca. HOP-62 | 5.4 |
| Lung ca. NCI-H522 | 34.6 |
| Liver | 0.0 |
| Fetal Liver | 33.2 |
| Liver ca. HepG2 | 12.8 |
| Kidney Pool | 0.1 |
| Fetal Kidney | 12.2 |
| Renal ca. 786-0 | 30.6 |
| Renal ca. A498 | 4.9 |
| Renal ca. ACHN | 12.9 |
| Renal ca. UO-31 | 17.3 |
| Renal ca. TK-10 | 24.0 |
| Bladder | 3.1 |
| Gastric ca. (liver met.) NCI-N87 | 5.4 |
| Gastric ca. KATO III | 97.9 |
| Colon ca. SW-948 | 24.8 |
| Colon ca. SW480 | 86.5 |
| Colon ca.* (SW480 met) SW620 | 37.6 |
| Colon ca. HT29 | 17.4 |
| Colon ca. HCT-116 | 100.0 |
| Colon ca. CaCo-2 | 31.4 |
| Colon cancer tissue | 7.0 |
| Colon ca. SW1116 | 16.8 |
| Colon ca. Colo-205 | 18.2 |
| Colon ca. SW-48 | 11.0 |
| Colon Pool | 0.6 |
| Small Intestine Pool | 0.2 |
| Stomach Pool | 0.2 |
| Bone Marrow Pool | 0.2 |
| Fetal Heart | 6.0 |
| Heart Pool | 0.3 |
| Lymph Node Pool | 0.6 |
| Fetal Skeletal Muscle | 3.0 |
| Skeletal Muscle Pool | 0.0 |
| Spleen Pool | 1.4 |
| Thymus Pool | 12.1 |
| CNS cancer (glio/astro) U87-MG | 19.1 |
| CNS cancer (glio/astro) U-118-MG | 97.9 |
| CNS cancer (neuro; met) SK-N-AS | 52.5 |
| CNS cancer (astro) SF-539 | 25.7 |
| CNS cancer (astro) SNB-75 | 66.0 |
| CNS cancer (glio) SNB-19 | 9.4 |
| CNS cancer (glio) SF-295 | 5.3 |
| Brain (Amygdala) Pool | 0.1 |
| Brain (cerebellum) | 0.1 |
| Brain (fetal) | 2.7 |
| Brain (Hippocampus) Pool | 0.2 |
| Cerebral Cortex Pool | 0.3 |
| Brain (Substantia nigra) Pool | 0.1 |
| Brain (Thalamus) Pool | 0.2 |
| Brain (whole) | 0.3 |
| Spinal Cord Pool | 0.1 |
| Adrenal Gland | 0.1 |
| Pituitary gland Pool | 0.1 |
| Salivary Gland | 0.3 |
| Thyroid (female) | 0.0 |
| Pancreatic ca. CAPAN2 | 41.8 |
| Pancreas Pool | 0.5 |

TABLE WC
Panel 4.1D

| | Rel. Exp. (%) |
|---|---|
| | Ag5695, Run |
| Tissue Name | 246504814 |
| Secondary Th1 act | 79.6 |
| Secondary Th2 act | 74.2 |
| Secondary Tr1 act | 18.9 |
| Secondary Th1 rest | 0.2 |
| Secondary Th2 rest | 0.3 |
| Secondary Tr1 rest | 0.0 |
| Primary Th1 act | 0.9 |
| Primary Th2 act | 38.4 |
| Primary Tr1 act | 30.8 |
| Primary Th1 rest | 2.0 |
| Primary Th2 rest | 4.2 |
| Primary Tr1 rest | 2.7 |
| CD45RA CD4 lymphocyte act | 52.5 |
| CD45RO CD4 lymphocyte act | 47.0 |
| CD8 lymphocyte act | 11.4 |
| Secondary CD8 lymphocyte rest | 24.1 |
| Secondary CD8 lymphocyte act | 4.4 |
| CD4 lymphocyte none | 0.0 |
| 2ry Th1/Th2/Tr1_anti-CD95 CH11 | 3.5 |
| LAK cells rest | 1.6 |
| LAK cells IL-2 | 8.7 |
| LAK cells IL-2 + IL-12 | 1.9 |
| LAK cells IL-2 + IFN gamma | 10.5 |
| LAK cells IL-2 + IL-18 | 6.3 |
| LAK cells PMA/ionomycin | 3.1 |
| NK Cells IL-2 rest | 81.2 |
| Two Way MLR 3 day | 1.9 |
| Two Way MLR 5 day | 2.9 |
| Two Way MLR 7 day | 9.2 |
| PBMC rest | 0.0 |
| PBMC PWM | 4.0 |
| PBMC PHA-L | 12.5 |
| Ramos (B cell) none | 8.1 |
| Ramos (B cell) ionomycin | 76.3 |
| B lymphocytes PWM | 52.9 |
| B lymphocytes CD40L and IL-4 | 49.7 |
| EOL-1 dbcAMP | 31.6 |
| EOL-1 dbcAMP PMA/ionomycin | 1.9 |
| Dendritic cells none | 0.6 |
| Dendritic cells LPS | 0.0 |
| Dendritic cells anti-CD40 | 0.0 |
| Monocytes rest | 0.0 |
| Monocytes LPS | 0.0 |
| Macrophages rest | 2.2 |
| Macrophages LPS | 0.2 |
| HUVEC none | 31.0 |
| HUVEC starved | 55.5 |
| HUVEC IL-1beta | 42.9 |
| HUVEC IFN gamma | 27.7 |
| HUVEC TNF alpha + IFN gamma | 5.7 |
| HUVEC TNF alpha + IL4 | 4.5 |
| HUVEC IL-11 | 23.2 |
| Lung Microvascular EC none | 24.8 |
| Lung Microvascular EC TNFalpha + IL-1beta | 1.9 |
| Microvascular Dermal EC none | 2.4 |
| Microsvasular Dermal EC TNFalpha + IL-1beta | 4.4 |
| Bronchial epithelium TNFalpha + IL1beta | 1.8 |
| Small airway epithelium none | 1.2 |
| Small airway epithelium TNFalpha + IL-1beta | 4.5 |
| Coronery artery SMC rest | 4.4 |
| Coronery artery SMC TNFalpha + IL-1beta | 3.2 |
| Astrocytes rest | 0.3 |
| Astrocytes TNFalpha + IL-1beta | 0.7 |
| KU-812 (Basophil) rest | 32.1 |
| KU-812 (Basophil) PMA/ionomycin | 42.3 |
| CCD1106 (Keratinocytes) none | 44.8 |
| CCD1106 (Keratinocytes) TNFalpha + IL-1beta | 9.1 |
| Liver cirrhosis | 2.7 |
| NCI-H292 none | 19.9 |
| NCI-H292 IL-4 | 42.9 |
| NCI-H292 IL-9 | 58.6 |
| NCI-H292 IL-13 | 52.5 |
| NCI-H292 IFN gamma | 20.3 |
| HPAEC none | 7.4 |
| HPAEC TNF alpha + IL-1 beta | 21.3 |
| Lung fibroblast none | 5.9 |
| Lung fibroblast TNF alpha + IL-1 beta | 8.9 |
| Lung fibroblast IL-4 | 0.8 |
| Lung fibroblast IL-9 | 5.8 |
| Lung fibroblast IL-13 | 0.4 |
| Lung fibroblast IFN gamma | 1.4 |
| Dermal fibroblast CCD1070 rest | 100.0 |
| Dermal fibroblast CCD1070 TNF alpha | 93.3 |
| Dermal fibroblast CCD1070 IL-1 beta | 40.3 |
| Dermal fibroblast IFN gamma | 27.9 |
| Dermal fibroblast IL-4 | 40.3 |
| Dermal Fibroblasts rest | 18.3 |
| Neutrophils TNFa + LPS | 0.0 |
| Neutrophils rest | 0.0 |
| Colon | 0.0 |
| Lung | 0.2 |
| Thymus | 8.5 |
| Kidney | 0.0 |

CNS_neurodegeneration_v1.0 Summary: Ag5695 Expression of this gene is low/undetectable (CTs>35) across all of the samples on this panel (data not shown).

General_screening_panel_v1.5 Summary: Ag5695 Highest expression of this gene is detected in a colon cancer HCT-116 cell line (CT=27). Moderate expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

In addition, significant expression of this gene is seen in fetal tissues, including fetal lung, liver, kidney, heart, and skeletal muscle. Expression of this gene is higher in fetal (CTs=28-32) compared to corresponding adult lung, liver, kidney, heart, and skeletal muscle tissues. Therefore, expression of this gene may be useful in distinguishing between fetal and adult lung, liver, kidney, heart, and skeletal muscle. In addition, expression in fetal tissue suggests a role for the protein encoded by this gene in growth and development of these tissues in the fetus and thus may also act in a regenerative capacity in the adult.

Panel 4.1D Summary: Ag5695 Highest expression of this gene is detected in dermal fibroblast (CT=29.2). Moderate to low levels of expression of this gene is detected in polarized T cells (primary and secondary Th1, Th2, and Tr1), activated CD45RA CD4 and CD45RO CD4 lymphocytes, LAK cells, resting IL-2 treated NK cells, activated PBMC cells, Ramos B cells, B lymphocytes, eosinophils, endothelial cells, basophils, NCI-H292 cells, lung and dermal fibroblasts and thymus. Interestingly, expression of this gene is upregulated in activated polarized T cells, stimulted PBMC cells, and activated Ramos B cells. Therefore, therapeutic modulation of this gene may be useful in the treatment of autoimmune and inflammatory disorders including psoriasis, allergy, asthma, inflammatory bowel disease, rheumatoid arthritis and osteoarthritis.

X. CG154492-01: High-Affiniti CGMP-Specific 3′,5′-Cyclic Phosphodiesterase 9A.

Expression of gene CG154492-01 was assessed using the primer-probe set Ag6818, described in Table XA. Results of the RTQ-PCR runs are shown in Table XB.

TABLE XA
Probe Name Ag6818

| | | | Start | SEQ ID |
|---|---|---|---|---|
| Primers | Sequence | Length | Position | No |
| Forward | 5′-gcagaaattatggattctttcaaag-3′ | 25 | 1345 | 540 |
| Probe | TET-5′-tcctcgttgctgtagtcaaaattctcca-3′-TAMRA | 28 | 1376 | 541 |
| Reverse | 5′-ggtcgctgagggtcatg-3′ | 17 | 1407 | 542 |

TABLE XB
General_screening_panel_v1.6

| | Rel. Exp. (%) |
|---|---|
| | Ag6818, Run |
| Tissue Name | 278391557 |
| Adipose | 18.4 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 10.5 |
| Squamous cell carcinoma SCC-4 | 0.0 |
| Testis Pool | 8.1 |
| Prostate ca.* (bone met) PC-3 | 0.0 |
| Prostate Pool | 24.3 |
| Placenta | 3.7 |
| Uterus Pool | 0.0 |
| Ovarian ca. OVCAR-3 | 53.6 |
| Ovarian ca. SK-OV-3 | 31.6 |
| Ovarian ca. OVCAR-4 | 9.4 |
| Ovarian ca. OVCAR-5 | 24.7 |
| Ovarian ca. IGROV-1 | 14.1 |
| Ovarian ca. OVCAR-8 | 4.3 |
| Ovary | 6.1 |
| Breast ca. MCF-7 | 3.0 |
| Breast ca. MDA-MB-231 | 0.0 |
| Breast ca. BT 549 | 0.0 |
| Breast ca. T47D | 0.0 |
| Breast ca. MDA-N | 5.6 |
| Breast Pool | 2.2 |
| Trachea | 2.8 |
| Lung | 0.0 |
| Fetal Lung | 33.2 |
| Lung ca. NCI-N417 | 0.0 |
| Lung ca. LX-1 | 3.1 |
| Lung ca. NCI-H146 | 3.5 |
| Lung ca. SHP-77 | 0.0 |
| Lung ca. A549 | 0.0 |
| Lung ca. NCI-H526 | 0.0 |
| Lung ca. NCI-H23 | 25.0 |
| Lung ca. NCI-H460 | 7.3 |
| Lung ca. HOP-62 | 0.0 |
| Lung ca. NCI-H522 | 65.1 |
| Liver | 0.0 |
| Fetal Liver | 4.4 |
| Liver ca. HepG2 | 31.9 |
| Kidney Pool | 27.2 |
| Fetal Kidney | 10.3 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 15.4 |
| Bladder | 0.0 |
| Gastric ca. (liver met.) NCI-N87 | 15.4 |
| Gastric ca. KATO III | 0.0 |
| Colon ca. SW-948 | 0.0 |
| Colon ca. SW480 | 19.6 |
| Colon ca.* (SW480 met) SW620 | 3.2 |
| Colon ca. HT29 | 3.6 |
| Colon ca. HCT-116 | 29.7 |
| Colon ca. CaCo-2 | 7.9 |
| Colon cancer tissue | 2.5 |
| Colon ca. SW1116 | 0.0 |
| Colon ca. Colo-205 | 0.0 |
| Colon ca. SW-48 | 0.0 |
| Colon Pool | 3.0 |
| Small Intestine Pool | 5.9 |
| Stomach Pool | 6.0 |
| Bone Marrow Pool | 0.0 |
| Fetal Heart | 9.8 |
| Heart Pool | 3.1 |
| Lymph Node Pool | 3.5 |
| Fetal Skeletal Muscle | 3.3 |
| Skeletal Muscle Pool | 0.0 |
| Spleen Pool | 2.8 |
| Thymus Pool | 6.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 24.0 |
| CNS cancer (glio) SF-295 | 0.0 |
| Brain (Amygdala) Pool | 3.7 |
| Brain (cerebellum) | 50.7 |
| Brain (fetal) | 100.0 |
| Brain (Hippocampus) Pool | 2.2 |
| Cerebral Cortex Pool | 7.6 |
| Brain (Substantia nigra) Pool | 11.8 |
| Brain (Thalamus) Pool | 15.3 |
| Brain (whole) | 20.2 |
| Spinal Cord Pool | 10.1 |
| Adrenal Gland | 16.6 |
| Pituitary gland Pool | 0.0 |
| Salivary Gland | 4.3 |
| Thyroid (female) | 0.0 |
| Pancreatic ca. CAPAN2 | 30.1 |
| Pancreas Pool | 14.0 |

CNS_neurodegeneration_v1.0 Summary: Ag6818 Expression of this gene is low/undetectable in all samples on this panel (CTs>35). (Data not shown.)

General_screening_panel_v1.6 Summary: Ag6818 Expression of this gene is limited to the fetal brain (CT=34.5). Thus, expression of this gene could be used to differentiate between fetal and adult brain tissue and as a marker of fetal neural tissue.

Panel 4.1D Summary: Ag6818 Expression of this gene is low/undetectable in all samples on this panel (CTs>35). (Data not shown.)

Panel 5 Islet Summary: Ag6818 Expression of this gene is low/undetectable in all samples on this panel (CTs>35). (Data not shown.)

Y. CG154509-01: Cytoplasmic Dynein Heavy Chain.

Expression of gene CG154509-01 was assessed using the primer-probe set Ag5696, described in Table YA. Results of the RTQ-PCR runs are shown in Tables YB, YC and YD.

TABLE YA
Probe Name Ag5696

| | | | Start | SEQ ID |
|---|---|---|---|---|
| Primers | Sequence | Length | Position | No |
| Forward | 5′-ccagattgaagtgatgaaagga-3′ | 22 | 3156 | 543 |
| Probe | TET-5′-cacgtcttcagatctattatcaagaactgg-3′-TAMRA | 30 | 3188 | 544 |
| Reverse | 5′-gtcccaacgagctttaaatttt-3′ | 22 | 3219 | 545 |

TABLE YB
AI_comprehensive panel_v1.0

| | Rel. Exp. (%) |
|---|---|
| | Ag5696, Run |
| Tissue Name | 245243119 |
| 110967 COPD-F | 20.3 |
| 110980 COPD-F | 5.1 |
| 110968 COPD-M | 21.3 |
| 110977 COPD-M | 24.7 |
| 110989 Emphysema-F | 8.3 |
| 110992 Emphysema-F | 16.5 |
| 110993 Emphysema-F | 18.2 |
| 110994 Emphysema-F | 8.6 |
| 110995 Emphysema-F | 15.2 |
| 110996 Emphysema-F | 8.5 |
| 110997 Asthma-M | 18.2 |
| 111001 Asthma-F | 4.5 |
| 111002 Asthma-F | 54.0 |
| 111003 Atopic Asthma-F | 20.6 |
| 111004 Atopic Asthma-F | 0.0 |
| 111005 Atopic Asthma-F | 17.2 |
| 111006 Atopic Asthma-F | 76.8 |
| 111417 Allergy-M | 85.3 |
| 112347 Allergy-M | 0.0 |
| 112349 Normal Lung-F | 5.1 |
| 112357 Normal Lung-F | 13.4 |
| 112354 Normal Lung-M | 89.5 |
| 112374 Crohns-F | 52.1 |
| 112389 Match Control Crohns-F | 47.6 |
| 112375 Crohns-F | 6.2 |
| 112732 Match Control Crohns-F | 17.7 |
| 112725 Crohns-M | 42.3 |
| 112387 Match Control Crohns-M | 18.6 |
| 112378 Crohns-M | 0.3 |
| 112390 Match Control Crohns-M | 19.2 |
| 112726 Crohns-M | 0.6 |
| 112731 Match Control Crohns-M | 4.7 |
| 112380 Ulcer Col-F | 48.3 |
| 112734 Match Control Ulcer Col-F | 9.1 |
| 112384 Ulcer Col-F | 13.2 |
| 112737 Match Control Ulcer Col-F | 23.5 |
| 112386 Ulcer Col-F | 24.1 |
| 112738 Match Control Ulcer Col-F | 26.4 |
| 112381 Ulcer Col-M | 5.6 |
| 112735 Match Control Ulcer Col-M | 14.5 |
| 112382 Ulcer Col-M | 37.1 |
| 112394 Match Control Ulcer Col-M | 7.1 |
| 112383 Ulcer Col-M | 21.9 |
| 112736 Match Control Ulcer Col-M | 44.1 |
| 112423 Psoriasis-F | 34.2 |
| 112427 Match Control Psoriasis-F | 21.0 |
| 112418 Psoriasis-M | 22.5 |
| 112723 Match Control Psoriasis-M | 61.1 |
| 112419 Psoriasis-M | 2.8 |
| 112424 Match Control Psoriasis-M | 24.7 |
| 112420 Psoriasis-M | 12.3 |
| 112425 Match Control Psoriasis-M | 25.9 |
| 104689 (MF) OA Bone-Backus | 29.5 |
| 104690 (MF) Adj “Normal” Bone-Backus | 0.6 |
| 104691 (MF) OA Synovium-Backus | 94.6 |
| 104692 (BA) OA Cartilage-Backus | 21.0 |
| 104694 (BA) OA Bone-Backus | 15.1 |
| 104695 (BA) Adj “Normal” Bone-Backus | 31.6 |
| 104696 (BA) OA Synovium-Backus | 11.4 |
| 104700 (SS) OA Bone-Backus | 10.5 |
| 104701 (SS) Adj “Normal” Bone-Backus | 100.0 |
| 104702 (SS) OA Synovium-Backus | 10.8 |
| 117093 OA Cartilage Rep7 | 9.2 |
| 112672 OA Bone5 | 4.9 |
| 112673 OA Synovium5 | 2.4 |
| 112674 OA Synovial Fluid cells5 | 12.4 |
| 117100 OA Cartilage Rep14 | 72.7 |
| 112756 OA Bone9 | 5.7 |
| 112757 OA Synovium9 | 0.9 |
| 112758 OA Synovial Fluid Cells9 | 21.5 |
| 117125 RA Cartilage Rep2 | 5.5 |
| 113492 Bone2 RA | 0.0 |
| 113493 Synovium2 RA | 10.1 |
| 113494 Syn Fluid Cells RA | 8.9 |
| 113499 Cartilage4 RA | 18.8 |
| 113500 Bone4 RA | 0.5 |
| 113501 Synovium4 RA | 5.0 |
| 113502 Syn Fluid Cells4 RA | 4.8 |
| 113495 Cartilage3 RA | 33.4 |
| 113496 Bone3 RA | 18.9 |
| 113497 Synovium3 RA | 3.9 |
| 113498 Syn Fluid Cells3 RA | 0.0 |
| 117106 Normal Cartilage Rep20 | 41.2 |
| 113663 Bone3 Normal | 31.6 |
| 113664 Synovium3 Normal | 18.3 |
| 113665 Syn Fluid Cells3 Normal | 80.1 |
| 117107 Normal Cartilage Rep22 | 13.3 |
| 113667 Bone4 Normal | 23.8 |
| 113668 Synovium4 Normal | 22.1 |
| 113669 Syn Fluid Cells4 Normal | 20.3 |

TABLE YC
CNS_neurodegeneration_v1.0

| | Rel. Exp. (%) | Rel. Exp. (%) |
|---|---|---|
| | Ag5696, Run | Ag5696, Run |
| Tissue Name | 247018771 | 312325348 |
| AD 1 Hippo | 9.7 | 45.4 |
| AD 2 Hippo | 33.0 | 93.3 |
| AD 3 Hippo | 17.1 | 43.2 |
| AD 4 Hippo | 24.5 | 42.0 |
| AD 5 hippo | 100.0 | 33.7 |
| AD 6 Hippo | 45.4 | 100.0 |
| Control 2 | 34.4 | 62.9 |
| Hippo | | |
| Control 4 | 27.5 | 26.2 |
| Hippo | | |
| Control (Path) | 24.8 | 25.9 |
| 3 Hippo | | |
| AD 1 Temporal | 42.9 | 28.1 |
| Ctx | | |
| AD 2 Temporal | 47.6 | 55.9 |
| Ctx | | |
| AD 3 Temporal | 23.5 | 48.3 |
| Ctx | | |
| AD 4 Temporal | 48.6 | 76.3 |
| Ctx | | |
| AD 5 Inf | 78.5 | 87.1 |
| Temporal Ctx | | |
| AD 5 | 50.0 | 45.7 |
| SupTemporal | | |
| Ctx | | |
| AD 6 Inf | 50.3 | 47.6 |
| Temporal Ctx | | |
| AD 6 Sup | 86.5 | 13.9 |
| Temporal Ctx | | |
| Control 1 | 21.6 | 21.2 |
| Temporal Ctx | | |
| Control 2 | 29.3 | 48.3 |
| Temporal Ctx | | |
| Control 3 | 30.6 | 51.4 |
| Temporal Ctx | | |
| Control 4 | 17.4 | 33.2 |
| Temporal Ctx | | |
| Control (Path) | 70.7 | 21.2 |
| 1 Temporal Ctx | | |
| Control (Path) | 44.8 | 32.1 |
| 2 Temporal Ctx | | |
| Control | 16.2 | 56.6 |
| (Path) 3 | | |
| Temporal | | |
| Ctx | | |
| Control | 76.8 | 27.7 |
| (Path) 4 | | |
| Temporal | | |
| Ctx | | |
| AD 1 | 48.6 | 49.3 |
| Occipital | | |
| Ctx | | |
| AD 2 | 0.0 | 78.5 |
| Occipital | | |
| Ctx | | |
| (Missing) | | |
| AD 3 | 20.9 | 33.9 |
| Occipital | | |
| Ctx | | |
| AD 4 | 48.3 | 50.3 |
| Occipital | | |
| Ctx | | |
| AD 5 | 32.1 | 25.0 |
| Occipital | | |
| Ctx | | |
| AD 6 | 46.7 | 43.2 |
| Occipital | | |
| Ctx | | |
| Control 1 | 14.3 | 45.4 |
| Occipital | | |
| Ctx | | |
| Control 2 | 43.8 | 37.1 |
| Occipital | | |
| Ctx | | |
| Control 3 | 57.8 | 31.6 |
| Occipital | | |
| Ctx | | |
| Control 4 | 20.3 | 39.5 |
| Occipital | | |
| Ctx | | |
| Control | 99.3 | 22.2 |
| (Path) 1 | | |
| Occipital | | |
| Ctx | | |
| Control | 31.6 | 51.8 |
| (Path) 2 | | |
| Occipital | | |
| Ctx | | |
| Control | 5.1 | 60.3 |
| (Path) 3 | | |
| Occipital | | |
| Ctx | | |
| Control | 69.7 | 20.9 |
| (Path) 4 | | |
| Occipital | | |
| Ctx | | |
| Control 1 | 23.3 | 29.9 |
| Parietal Ctx | | |
| Control 2 | 56.3 | 37.4 |
| Parietal Ctx | | |
| Control 3 | 16.8 | 45.7 |
| Parietal Ctx | | |
| Control | 82.4 | 37.4 |
| (Path) 1 | | |
| Parietal Ctx | | |
| Control | 49.3 | 58.6 |
| (Path) 2 | | |
| Parietal Ctx | | |
| Control | 14.4 | 0.3 |
| (Path) 3 | | |
| Parietal Ctx | | |
| Control | 71.7 | 7.6 |
| (Path) 4 | | |
| Parietal Ctx | | |

TABLE YD
Panel 4.1D

| | Rel. Exp. (%) |
|---|---|
| | Ag5696, Run |
| Tissue Name | 246509228 |
| Secondary Th1 act | 0.9 |
| Secondary Th2 act | 0.2 |
| Secondary Tr1 act | 0.5 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.3 |
| Primary Tr1 act | 0.0 |
| Primary Th1 rest | 0.0 |
| Primary Th2 rest | 0.3 |
| Primary Tr1 rest | 0.0 |
| CD45RA CD4 lymphocyte act | 25.9 |
| CD45RO CD4 lymphocyte act | 5.6 |
| CD8 lymphocyte act | 0.6 |
| Secondary CD8 lymphocyte rest | 3.7 |
| Secondary CD8 lymphocyte act | 0.3 |
| CD4 lymphocyte none | 0.4 |
| 2ry Th1/Th2/Tr1_anti-CD95 CH11 | 0.6 |
| LAK cells rest | 3.0 |
| LAK cells IL-2 | 2.6 |
| LAK cells IL-2 + IL-12 | 0.8 |
| LAK cells IL-2 + IFN gamma | 2.0 |
| LAK cells IL-2 + IL-18 | 1.1 |
| LAK cells PMA/ionomycin | 1.8 |
| NK Cells IL-2 rest | 11.3 |
| Two Way MLR 3 day | 0.7 |
| Two Way MLR 5 day | 0.0 |
| Two Way MLR 7 day | 0.4 |
| PBMC rest | 0.5 |
| PBMC PWM | 0.2 |
| PBMC PHA-L | 1.4 |
| Ramos (B cell) none | 0.0 |
| Ramos (B cell) ionomycin | 0.4 |
| B lymphocytes PWM | 0.8 |
| B lymphocytes CD40L and IL-4 | 0.3 |
| EOL-1 dbcAMP | 3.7 |
| EOL-1 dbcAMP PMA/ionomycin | 0.3 |
| Dendritic cells none | 1.3 |
| Dendritic cells LPS | 0.0 |
| Dendritic cells anti-CD40 | 0.0 |
| Monocytes rest | 0.5 |
| Monocytes LPS | 1.5 |
| Macrophages rest | 0.2 |
| Macrophages LPS | 0.4 |
| HUVEC none | 5.7 |
| HUVEC starved | 4.5 |
| HUVEC IL-1beta | 7.6 |
| HUVEC IFN gamma | 12.8 |
| HUVEC TNF alpha + IFN gamma | 0.9 |
| HUVEC TNF alpha + IL4 | 0.8 |
| HUVEC IL-11 | 8.1 |
| Lung Microvascular EC none | 17.1 |
| Lung Microvascular EC TNFalpha + IL-1beta | 6.8 |
| Microvascular Dermal EC none | 1.0 |
| Microsvasular Dermal EC TNFalpha + IL-1beta | 3.4 |
| Bronchial epithelium TNFalpha + IL1beta | 4.5 |
| Small airway epithelium none | 5.7 |
| Small airway epithelium TNFalpha + IL-1beta | 10.4 |
| Coronery artery SMC rest | 21.5 |
| Coronery artery SMC TNFalpha + IL-1beta | 20.7 |
| Astrocytes rest | 3.8 |
| Astrocytes TNFalpha + IL-1beta | 2.0 |
| KU-812 (Basophil) rest | 7.8 |
| KU-812 (Basophil) PMA/ionomycin | 8.3 |
| CCD1106 (Keratinocytes) none | 37.4 |
| CCD1106 (Keratinocytes) TNFalpha + IL-1beta | 11.0 |
| Liver cirrhosis | 14.8 |
| NCI-H292 none | 44.1 |
| NCI-H292 IL-4 | 37.6 |
| NCI-H292 IL-9 | 100.0 |
| NCI-H292 IL-13 | 44.8 |
| NCI-H292 IFN gamma | 17.2 |
| HPAEC none | 4.2 |
| HPAEC TNF alpha + IL-1 beta | 33.0 |
| Lung fibroblast none | 79.6 |
| Lung fibroblast TNF alpha + IL-1 beta | 48.3 |
| Lung fibroblast IL-4 | 12.7 |
| Lung fibroblast IL-9 | 37.1 |
| Lung fibroblast IL-13 | 6.3 |
| Lung fibroblast IFN gamma | 37.6 |
| Dermal fibroblast CCD1070 rest | 58.2 |
| Dermal fibroblast CCD1070 TNF alpha | 46.0 |
| Dermal fibroblast CCD1070 IL-1 beta | 39.2 |
| Dermal fibroblast IFN gamma | 28.1 |
| Dermal fibroblast IL-4 | 88.3 |
| Dermal Fibroblasts rest | 35.1 |
| Neutrophils TNFa + LPS | 0.0 |
| Neutrophils rest | 0.3 |
| Colon | 0.6 |
| Lung | 1.2 |
| Thymus | 2.0 |
| Kidney | 59.0 |

AI_comprehensive panel_v1.0 Summary: Ag5696 Highest expression of this gene is seen in a normal bone sample adjacent to OA bone (CT=28). Overall, this gene is widely expressed on this panel, with moderate levels of expression in a wide range of tissues and samples related to autoimmune disease. Thus, modulation of the expression or function of this gene may be useful in the treatment of autoimmune diseases, including RA, OA, allergy, emphysema and asthma.

CNS_neurodegeneration_v1.0 Summary: Ag5696 Two experiments with the same probe and primer set produce results that are in very good agreement. This panel does not show differential expression of this gene in Alzheimer's disease. However, this panel does show that this gene is expressed at high to moderate levels in the hippocampus and cerebral cortex. Thus, therapeutic modulation of the expression or function of this gene may be useful in the treatment of neurological disorders, such as Alzheimer's disease, Parkinson's disease, schizophrenia, multiple sclerosis, stroke and epilepsy.

Panel 4.1D Summary: Ag5696 Highest expression of this gene is seen in IL-9 treated NCI-H292 goblet cells. Moderate levels of expression are seen in clusters of samples derived from lung and dermal fibroblasts. Low but significant levels of expression are seen in endothelial cells from the lung and skin, as well as small airway and bronchial epithelium. The prominent expression in cells and cell lines derived from the lung and skin suggest that this gene product may be involved in inflammatory conditions of the lung and skin, including psoriasis, asthma, emphysema, allergy, and chronic obstructive pulmonary disease.

Z. CG155595-01: Kinesin 7.

Expression of gene CG155595-01 was assessed using the primer-probe set Ag5284, described in Table ZA. Results of the RTQ-PCR runs are shown in Tables ZB, ZC, ZD and ZE.

TABLE ZA
Probe Name Ag5284

| | | | Start | SEQ ID |
|---|---|---|---|---|
| Primers | Sequence | Length | Position | No |
| Forward | 5′-gatcagaggacctcgaggaa-3′ | 20 | 3979 | 546 |
| Probe | TET-5′-ccacatgcacaaggattattccatacca-3′-TAMRA | 28 | 3999 | 547 |
| Reverse | 5′-agaagctgcctgtctccttaat-3′ | 22 | 4043 | 548 |

TABLE ZB
AI_comprehensive panel_v1.0

| | Rel. Exp. (%) |
|---|---|
| | Ag5284, Run |
| Tissue Name | 234222219 |
| 110967 COPD-F | 8.5 |
| 110980 COPD-F | 15.5 |
| 110968 COPD-M | 17.8 |
| 110977 COPD-M | 77.4 |
| 110989 Emphysema-F | 38.4 |
| 110992 Emphysema-F | 3.3 |
| 110993 Emphysema-F | 16.8 |
| 110994 Emphysema-F | 8.8 |
| 110995 Emphysema-F | 26.8 |
| 110996 Emphysema-F | 5.3 |
| 110997 Asthma-M | 10.0 |
| 111001 Asthma-F | 5.7 |
| 111002 Asthma-F | 18.9 |
| 111003 Atopic Asthma-F | 18.8 |
| 111004 Atopic Asthma-F | 22.1 |
| 111005 Atopic Asthma-F | 13.7 |
| 111006 Atopic Asthma-F | 2.8 |
| 111417 Allergy-M | 2.0 |
| 112347 Allergy-M | 6.3 |
| 112349 Normal Lung-F | 10.4 |
| 112357 Normal Lung-F | 87.7 |
| 112354 Normal Lung-M | 49.7 |
| 112374 Crohns-F | 21.0 |
| 112389 Match Control Crohns-F | 15.6 |
| 112375 Crohns-F | 10.1 |
| 112732 Match Control Crohns-F | 3.0 |
| 112725 Crohns-M | 9.6 |
| 112387 Match Control Crohns-M | 3.1 |
| 112378 Crohns-M | 15.2 |
| 112390 Match Control Crohns-M | 73.2 |
| 112726 Crohns-M | 12.8 |
| 112731 Match Control Crohns-M | 32.1 |
| 112380 Ulcer Col-F | 23.3 |
| 112734 Match Control Ulcer Col-F | 21.3 |
| 112384 Ulcer Col-F | 33.9 |
| 112737 Match Control Ulcer Col-F | 9.0 |
| 112386 Ulcer Col-F | 2.3 |
| 112738 Match Control Ulcer Col-F | 6.5 |
| 112381 Ulcer Col-M | 6.1 |
| 112735 Match Control Ulcer Col-M | 34.2 |
| 112382 Ulcer Col-M | 23.8 |
| 112394 Match Control Ulcer Col-M | 3.4 |
| 112383 Ulcer Col-M | 14.0 |
| 112736 Match Control Ulcer Col-M | 8.9 |
| 112423 Psoriasis-F | 45.4 |
| 112427 Match Control Psoriasis-F | 100.0 |
| 112418 Psoriasis-M | 43.2 |
| 112723 Match Control Psoriasis-M | 14.6 |
| 112419 Psoriasis-M | 36.3 |
| 112424 Match Control Psoriasis-M | 23.2 |
| 112420 Psoriasis-M | 37.6 |
| 112425 Match Control Psoriasis-M | 66.9 |
| 104689 (MF) OA Bone-Backus | 23.8 |
| 104690 (MF) Adj “Normal” Bone-Backus | 19.2 |
| 104691 (MF) OA Synovium-Backus | 21.5 |
| 104692 (BA) OA Cartilage-Backus | 14.4 |
| 104694 (BA) OA Bone-Backus | 20.6 |
| 104695 (BA) Adj “Normal” Bone-Backus | 10.3 |
| 104696 (BA) OA Synovium-Backus | 9.5 |
| 104700 (SS) OA Bone-Backus | 11.4 |
| 104701 (SS) Adj “Normal” Bone-Backus | 6.0 |
| 104702 (SS) OA Synovium-Backus | 14.8 |
| 117093 OA Cartilage Rep7 | 9.6 |
| 112672 OA Bone5 | 49.0 |
| 112673 OA Synovium5 | 20.3 |
| 112674 OA Synovial Fluid cells5 | 13.6 |
| 117100 OA Cartilage Rep14 | 2.0 |
| 112756 OA Bone9 | 29.7 |
| 112757 OA Synovium9 | 5.4 |
| 112758 OA Synovial Fluid Cells9 | 17.0 |
| 117125 RA Cartilage Rep2 | 8.7 |
| 113492 Bone2 RA | 4.7 |
| 113493 Synovium2 RA | 0.0 |
| 113494 Syn Fluid Cells RA | 5.9 |
| 113499 Cartilage4 RA | 4.0 |
| 113500 Bone4 RA | 16.8 |
| 113501 Synovium4 RA | 2.5 |
| 113502 Syn Fluid Cells4 RA | 7.1 |
| 113495 Cartilage3 RA | 4.0 |
| 113496 Bone3 RA | 8.4 |
| 113497 Synovium3 RA | 0.0 |
| 113498 Syn Fluid Cells3 RA | 5.2 |
| 117106 Normal Cartilage Rep20 | 5.1 |
| 113663 Bone3 Normal | 9.2 |
| 113664 Synovium3 Normal | 3.8 |
| 113665 Syn Fluid Cells3 Normal | 14.7 |
| 117107 Normal Cartilage Rep22 | 0.0 |
| 113667 Bone4 Normal | 17.9 |
| 113668 Synovium4 Normal | 25.2 |
| 113669 Syn Fluid Cells4 Normal | 24.7 |

TABLE ZC
CNS_neurodegeneration_v1.0

| | Rel. Exp. (%) |
|---|---|
| | Ag5284, Run |
| Tissue Name | 233610763 |
| AD 1 Hippo | 17.6 |
| AD 2 Hippo | 0.0 |
| AD 3 Hippo | 6.7 |
| AD 4 Hippo | 0.0 |
| AD 5 hippo | 47.0 |
| AD 6 Hippo | 19.6 |
| Control 2 Hippo | 7.0 |
| Control 4 Hippo | 15.7 |
| Control (Path) 3 Hippo | 6.7 |
| AD 1 Temporal Ctx | 0.0 |
| AD 2 Temporal Ctx | 26.6 |
| AD 3 Temporal Ctx | 4.8 |
| AD 4 Temporal Ctx | 19.1 |
| AD 5 Inf Temporal Ctx | 100.0 |
| AD 5 Sup Temporal Ctx | 35.8 |
| AD 6 Inf Temporal Ctx | 15.7 |
| AD 6 Sup Temporal Ctx | 20.2 |
| Control 1 Temporal Ctx | 18.3 |
| Control 2 Temporal Ctx | 12.7 |
| Control 3 Temporal Ctx | 0.0 |
| Control 4 Temporal Ctx | 15.1 |
| Control (Path) 1 Temporal Ctx | 38.4 |
| Control (Path) 2 Temporal Ctx | 38.7 |
| Control (Path) 3 Temporal Ctx | 0.0 |
| Control (Path) 4 Temporal Ctx | 29.5 |
| AD 1 Occipital Ctx | 0.0 |
| AD 2 Occipital Ctx (Missing) | 0.0 |
| AD 3 Occipital Ctx | 6.9 |
| AD 4 Occipital Ctx | 8.8 |
| AD 5 Occipital Ctx | 6.3 |
| AD 6 Occipital Ctx | 12.2 |
| Control 1 Occipital Ctx | 5.9 |
| Control 2 Occipital Ctx | 35.1 |
| Control 3 Occipital Ctx | 42.0 |
| Control 4 Occipital Ctx | 0.0 |
| Control (Path) 1 Occipital Ctx | 10.3 |
| Control (Path) 2 Occipital Ctx | 7.2 |
| Control (Path) 3 Occipital Ctx | 0.0 |
| Control (Path) 4 Occipital Ctx | 15.6 |
| Control 1 Parietal Ctx | 4.2 |
| Control 2 Parietal Ctx | 18.8 |
| Control 3 Parietal Ctx | 10.5 |
| Control (Path) 1 Parietal Ctx | 17.3 |
| Control (Path) 2 Parietal Ctx | 8.2 |
| Control (Path) 3 Parietal Ctx | 0.0 |
| Control (Path) 4 Parietal Ctx | 34.9 |

TABLE ZD
General_screening_panel_v1.5

| | Rel. Exp. (%) |
|---|---|
| | Ag5284, Run |
| Tissue Name | 230564176 |
| Adipose | 2.5 |
| Melanoma* Hs688(A).T | 17.4 |
| Melanoma* Hs688(B).T | 28.1 |
| Melanoma* M14 | 32.8 |
| Melanoma* LOXIMVI | 23.3 |
| Melanoma* SK-MEL-5 | 18.0 |
| Squamous cell carcinoma SCC-4 | 12.7 |
| Testis Pool | 1.6 |
| Prostate ca.* (bone met) PC-3 | 9.5 |
| Prostate Pool | 1.5 |
| Placenta | 0.5 |
| Uterus Pool | 2.2 |
| Ovarian ca. OVCAR-3 | 18.6 |
| Ovarian ca. SK-OV-3 | 48.6 |
| Ovarian ca. OVCAR-4 | 11.3 |
| Ovarian ca. OVCAR-5 | 51.4 |
| Ovarian ca. IGROV-1 | 8.4 |
| Ovarian ca. OVCAR-8 | 15.8 |
| Ovary | 4.2 |
| Breast ca. MCF-7 | 19.3 |
| Breast ca. MDA-MB-231 | 37.9 |
| Breast ca. BT 549 | 16.6 |
| Breast ca. T47D | 9.7 |
| Breast ca. MDA-N | 24.7 |
| Breast Pool | 7.1 |
| Trachea | 1.4 |
| Lung | 21.2 |
| Fetal Lung | 15.1 |
| Lung ca. NCI-N417 | 6.0 |
| Lung ca. LX-1 | 20.3 |
| Lung ca. NCI-H146 | 2.8 |
| Lung ca. SHP-77 | 44.1 |
| Lung ca. A549 | 46.7 |
| Lung ca. NCI-H526 | 5.0 |
| Lung ca. NCI-H23 | 88.9 |
| Lung ca. NCI-H460 | 11.4 |
| Lung ca. HOP-62 | 13.4 |
| Lung ca. NCI-H522 | 30.4 |
| Liver | 0.0 |
| Fetal Liver | 24.0 |
| Liver ca. HepG2 | 12.0 |
| Kidney Pool | 24.1 |
| Fetal Kidney | 45.7 |
| Renal ca. 786-0 | 18.3 |
| Renal ca. A498 | 6.2 |
| Renal ca. ACHN | 5.7 |
| Renal ca. UO-31 | 7.5 |
| Renal ca. TK-10 | 23.7 |
| Bladder | 6.1 |
| Gastric ca. (liver met.) NCI-N87 | 60.3 |
| Gastric ca. KATO III | 36.9 |
| Colon ca. SW-948 | 6.3 |
| Colon ca. SW480 | 41.2 |
| Colon ca.* (SW480 met) SW620 | 22.7 |
| Colon ca. HT29 | 10.4 |
| Colon ca. HCT-116 | 100.0 |
| Colon ca. CaCo-2 | 54.0 |
| Colon cancer tissue | 8.3 |
| Colon ca. SW1116 | 7.3 |
| Colon ca. Colo-205 | 5.3 |
| Colon ca. SW-48 | 5.7 |
| Colon Pool | 3.6 |
| Small Intestine Pool | 15.8 |
| Stomach Pool | 3.7 |
| Bone Marrow Pool | 4.2 |
| Fetal Heart | 5.4 |
| Heart Pool | 1.5 |
| Lymph Node Pool | 12.2 |
| Fetal Skeletal Muscle | 5.1 |
| Skeletal Muscle Pool | 0.4 |
| Spleen Pool | 2.6 |
| Thymus Pool | 13.8 |
| CNS cancer (glio/astro) U87-MG | 36.3 |
| CNS cancer (glio/astro) U-118-MG | 80.7 |
| CNS cancer (neuro; met) SK-N-AS | 46.3 |
| CNS cancer (astro) SF-539 | 12.0 |
| CNS cancer (astro) SNB-75 | 37.1 |
| CNS cancer (glio) SNB-19 | 5.1 |
| CNS cancer (glio) SF-295 | 58.2 |
| Brain (Amygdala) Pool | 0.3 |
| Brain (cerebellum) | 0.3 |
| Brain (fetal) | 10.4 |
| Brain (Hippocampus) Pool | 0.6 |
| Cerebral Cortex Pool | 1.3 |
| Brain (Substantia nigra) Pool | 0.6 |
| Brain (Thalamus) Pool | 2.3 |
| Brain (whole) | 1.5 |
| Spinal Cord Pool | 1.9 |
| Adrenal Gland | 0.3 |
| Pituitary gland Pool | 0.7 |
| Salivary Gland | 0.5 |
| Thyroid (female) | 1.4 |
| Pancreatic ca. CAPAN2 | 31.0 |
| Pancreas Pool | 4.9 |

TABLE ZE
Panel 4.1D

| | Rel. Exp. (%) |
|---|---|
| | Ag5284, Run |
| Tissue Name | 230510205 |
| Secondary Th1 act | 37.9 |
| Secondary Th2 act | 40.6 |
| Secondary Tr1 act | 12.2 |
| Secondary Th1 rest | 0.0 |
| Secondary Th2 rest | 2.1 |
| Secondary Tr1 rest | 7.7 |
| Primary Th1 act | 5.4 |
| Primary Th2 act | 12.7 |
| Primary Tr1 act | 13.1 |
| Primary Th1 rest | 0.0 |
| Primary Th2 rest | 6.5 |
| Primary Tr1 rest | 6.0 |
| CD45RA CD4 lymphocyte act | 40.3 |
| CD45RO CD4 lymphocyte act | 31.9 |
| CD8 lymphocyte act | 19.5 |
| Secondary CD8 lymphocyte rest | 12.2 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 1.2 |
| LAK cells IL-2 | 13.0 |
| LAK cells IL-2 + IL-12 | 2.2 |
| LAK cells IL-2 + IFN gamma | 9.3 |
| LAK cells IL-2 + IL-18 | 2.2 |
| LAK cells PMA/ionomycin | 1.9 |
| NK Cells IL-2 rest | 47.6 |
| Two Way MLR 3 day | 3.4 |
| Two Way MLR 5 day | 2.5 |
| Two Way MLR 7 day | 9.4 |
| PBMC rest | 0.0 |
| PBMC PWM | 3.3 |
| PBMC PHA-L | 19.8 |
| Ramos (B cell) none | 11.9 |
| Ramos (B cell) ionomycin | 17.8 |
| B lymphocytes PWM | 13.7 |
| B lymphocytes CD40L and IL-4 | 18.3 |
| EOL-1 dbcAMP | 24.0 |
| EOL-1 dbcAMP PMA/ionomycin | 21.6 |
| Dendritic cells none | 1.6 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 3.1 |
| HUVEC starved | 22.7 |
| HUVEC IL-1beta | 14.6 |
| HUVEC IFN gamma | 18.8 |
| HUVEC TNF alpha + IFN gamma | 6.6 |
| HUVEC TNF alpha + IL4 | 5.3 |
| HUVEC IL-11 | 3.2 |
| Lung Microvascular EC none | 17.0 |
| Lung Microvascular EC TNFalpha + IL-1beta | 1.7 |
| Microvascular Dermal EC none | 8.7 |
| Microsvasular Dermal EC TNFalpha + IL-1beta | 1.3 |
| Bronchial epithelium TNFalpha + IL1beta | 0.0 |
| Small airway epithelium none | 0.0 |
| Small airway epithelium TNFalpha + IL-1beta | 8.7 |
| Coronery artery SMC rest | 0.0 |
| Coronery artery SMC TNFalpha + IL-1beta | 4.8 |
| Astrocytes rest | 4.1 |
| Astrocytes TNFalpha + IL-1beta | 3.7 |
| KU-812 (Basophil) rest | 33.9 |
| KU-812 (Basophil) PMA/ionomycin | 37.4 |
| CCD1106 (Keratinocytes) none | 31.9 |
| CCD1106 (Keratinocytes) TNFalpha + IL-1beta | 5.0 |
| Liver cirrhosis | 3.9 |
| NCI-H292 none | 36.6 |
| NCI-H292 IL-4 | 46.0 |
| NCI-H292 IL-9 | 73.2 |
| NCI-H292 IL-13 | 72.7 |
| NCI-H292 IFN gamma | 28.1 |
| HPAEC none | 2.8 |
| HPAEC TNF alpha + IL-1 beta | 11.1 |
| Lung fibroblast none | 9.2 |
| Lung fibroblast TNF alpha + IL-1 beta | 7.0 |
| Lung fibroblast IL-4 | 3.4 |
| Lung fibroblast IL-9 | 11.8 |
| Lung fibroblast IL-13 | 1.3 |
| Lung fibroblast IFN gamma | 5.5 |
| Dermal fibroblast CCD1070 rest | 20.9 |
| Dermal fibroblast CCD1070 TNF alpha | 100.0 |
| Dermal fibroblast CCD1070 IL-1 beta | 24.1 |
| Dermal fibroblast IFN gamma | 12.3 |
| Dermal fibroblast IL-4 | 38.7 |
| Dermal Fibroblasts rest | 7.2 |
| Neutrophils TNFa + LPS | 0.0 |
| Neutrophils rest | 0.0 |
| Colon | 0.0 |
| Lung | 0.0 |
| Thymus | 4.0 |
| Kidney | 0.0 |

AI_comprehensive panel_v1.0 Summary: Ag5284 Highest expression of this gene is seen in a normal tissue sample adjacent to psoriatic tissue (CT=33).

CNS_neurodegeneration_v1.0 Summary: Ag5284 Expression is limited to a single inferior temporal cortex sample from an Alzheimer's patient (CT=34.9).

General_screening_panel_v1.5 Summary: Ag5284 Highest expression is seen in a colon cancer cell line (CT=31). Prominent levels of expression are also seen in cell lines derived from brain, lung, colon, gastric, pancreatic, breast, ovarian, and melanoma cancers. Thus, expression of this gene could be used as a marker to detect the presence of these cancers. Furthermore, therapeutic modulation of the expression or function of this gene may be effective in the treatment of brain, lung, colon, gastric, pancreatic, breast, ovarian, and melanoma cancers.

Panel 4.1D Summary: Ag5284 Highest expression of this gene is seen in TNF-a treated dermal fibroblasts (CT=33). Low but significant levels of expression are also seen in clusters of samples derived from basophils, NCI-H292 cells, resting NK cells, and secondary activated T cells.

AA. CG157477-01: Myosin I.

Expression of gene CG157477-01 was assessed using the primer-probe set Ag5289, described in Table AAA. Results of the RTQ-PCR runs are shown in Tables AAB, AAC and AAD.

TABLE AAA
Probe Name Ag5289

| | | | Start | SEQ ID |
|---|---|---|---|---|
| Primers | Sequence | Length | Position | No |
| Forward | 5′-cgcatctatacgttcattgga-3′ | 21 | 151 | 549 |
| Probe | TET-5′-tcgtcgtttctgtgaacccttacaag-3′-TAMRA | 26 | 176 | 550 |
| Reverse | 5′-tgctcaattgtgtctcttccat-3′ | 22 | 215 | 551 |

TABLE AAB
CNS_neurodegeneration_v1.0

| | Rel. Exp. (%) |
|---|---|
| | Ag5289, Run |
| Tissue Name | 233610765 |
| AD 1 Hippo | 14.0 |
| AD 2 Hippo | 29.9 |
| AD 3 Hippo | 12.9 |
| AD 4 Hippo | 12.5 |
| AD 5 Hippo | 49.0 |
| AD 6 Hippo | 42.9 |
| Control 2 Hippo | 37.1 |
| Control 4 Hippo | 24.1 |
| Control (Path) 3 Hippo | 10.7 |
| AD 1 Temporal Ctx | 36.3 |
| AD 2 Temporal Ctx | 37.9 |
| AD 3 Temporal Ctx | 10.4 |
| AD 4 Temporal Ctx | 29.7 |
| AD 5 Inf Temporal Ctx | 83.5 |
| AD 5 Sup Temporal Ctx | 36.1 |
| AD 6 Inf Temporal Ctx | 61.1 |
| AD 6 Sup Temporal Ctx | 47.0 |
| Control 1 Temporal Ctx | 7.7 |
| Control 2 Temporal Ctx | 38.7 |
| Control 3 Temporal Ctx | 18.8 |
| Control 3 Temporal Ctx | 9.2 |
| Control (Path) 1 Temporal Ctx | 53.6 |
| Control (Path) 2 Temporal Ctx | 32.5 |
| Control (Path) 3 Temporal Ctx | 3.9 |
| Control (Path) 4 Temporal Ctx | 28.1 |
| AD 1 Occipital Ctx | 24.8 |
| AD 2 Occipital Ctx (Missing) | 0.0 |
| AD 3 Occipital Ctx | 11.5 |
| AD 4 Occipital Ctx | 25.2 |
| AD 5 Occipital Ctx | 44.1 |
| AD 6 Occipital Ctx | 22.5 |
| Control 1 Occipital Ctx | 8.1 |
| Control 2 Occipital Ctx | 49.7 |
| Control 3 Occipital Ctx | 19.9 |
| Control 4 Occipital Ctx | 15.8 |
| Control (Path) 1 Occipital Ctx | 100.0 |
| Control (Path) 2 Occipital Ctx | 25.5 |
| Control (Path) 3 Occipital Ctx | 4.2 |
| Control (Path) 4 Occipital Ctx | 20.3 |
| Control 1 Parietal Ctx | 17.3 |
| Control 2 Parietal Ctx | 39.0 |
| Control 3 Parietal Ctx | 21.5 |
| Control (Path) 1 Parietal Ctx | 50.0 |
| Control (Path) 2 Parietal Ctx | 39.5 |
| Control (Path) 3 Parietal Ctx | 4.1 |
| Control (Path) 4 Parietal Ctx | 38.2 |

TABLE AAC
General_screening_panel_v1.5

| | Rel. Exp. (%) |
|---|---|
| | Ag5289, Run |
| Tissue Name | 233238980 |
| Adipose | 7.2 |
| Melanoma* Hs688(A).T | 65.1 |
| Melanoma* Hs688(B).T | 16.2 |
| Melanoma* M14 | 23.3 |
| Melanoma* LOXIMVI | 8.1 |
| Melanoma* SK-MEL-5 | 11.2 |
| Squamous cell carcinoma SCC-4 | 3.1 |
| Testis Pool | 4.0 |
| Prostate ca.* (bone met) PC-3 | 28.7 |
| Prostate Pool | 7.4 |
| Placenta | 5.9 |
| Uterus Pool | 9.7 |
| Ovarian ca. OVCAR-3 | 2.1 |
| Ovarian ca. SK-OV-3 | 17.3 |
| Ovarian ca. OVCAR-4 | 6.0 |
| Ovarian ca. OVCAR-5 | 34.9 |
| Ovarian ca. IGROV-1 | 1.5 |
| Ovarian ca. OVCAR-8 | 1.6 |
| Ovary | 5.6 |
| Breast ca. MCF-7 | 11.6 |
| Breast ca. MDA-MB-231 | 0.5 |
| Breast ca. BT 549 | 0.1 |
| Breast ca. T47D | 17.6 |
| Breast ca. MDA-N | 4.4 |
| Breast Pool | 8.5 |
| Trachea | 17.6 |
| Lung | 3.1 |
| Fetal Lung | 15.4 |
| Lung ca. NCI-N417 | 1.8 |
| Lung ca. LX-1 | 34.2 |
| Lung ca. NCI-H146 | 8.2 |
| Lung ca. SHP-77 | 5.6 |
| Lung ca. A549 | 2.6 |
| Lung ca. NCI-H526 | 2.0 |
| Lung ca. NCI-H23 | 1.7 |
| Lung ca. NCI-H460 | 0.7 |
| Lung ca. HOP-62 | 1.6 |
| Lung ca. NCI-H522 | 0.6 |
| Liver | 0.9 |
| Fetal Liver | 10.4 |
| Liver ca. HepG2 | 13.3 |
| Kidney Pool | 15.0 |
| Fetal Kidney | 4.9 |
| Renal ca. 786-0 | 1.5 |
| Renal ca. A498 | 2.2 |
| Renal ca. ACHN | 28.1 |
| Renal ca. UO-31 | 7.0 |
| Renal ca. TK-10 | 14.0 |
| Bladder | 19.6 |
| Gastric ca. (liver met.) NCI-N87 | 21.3 |
| Gastric ca. KATO III | 50.3 |
| Colon ca. SW-948 | 1.5 |
| Colon ca. SW480 | 100.0 |
| Colon ca.* (SW480 met) SW620 | 12.9 |
| Colon ca. HT29 | 9.5 |
| Colon ca. HCT-116 | 11.8 |
| Colon ca. CaCo-2 | 66.9 |
| Colon cancer tissue | 19.5 |
| Colon ca. SW1116 | 3.4 |
| Colon ca. Colo-205 | 3.2 |
| Colon ca. SW-48 | 11.6 |
| Colon Pool | 9.0 |
| Small Intestine Pool | 6.3 |
| Stomach Pool | 3.7 |
| Bone Marrow Pool | 5.3 |
| Fetal Heart | 1.2 |
| Heart Pool | 3.6 |
| Lymph Node Pool | 10.4 |
| Fetal Skeletal Muscle | 0.7 |
| Skeletal Muscle Pool | 2.4 |
| Spleen Pool | 5.7 |
| Thymus Pool | 5.8 |
| CNS cancer (glio/astro) U87-MG | 5.6 |
| CNS cancer (glio/astro) U-118-MG | 1.5 |
| CNS cancer (neuro; met) SK-N-AS | 0.2 |
| CNS cancer (astro) SF-539 | 0.2 |
| CNS cancer (astro) SNB-75 | 0.1 |
| CNS cancer (glio) SNB-19 | 1.2 |
| CNS cancer (glio) SF-295 | 0.6 |
| Brain (Amygdala) Pool | 6.3 |
| Brain (cerebellum) | 11.0 |
| Brain (fetal) | 4.5 |
| Brain (Hippocampus) Pool | 6.2 |
| Cerebral Cortex Pool | 7.3 |
| Brain (Substantia nigra) Pool | 4.7 |
| Brain (Thalamus) Pool | 7.7 |
| Brain (whole) | 6.4 |
| Spinal Cord Pool | 12.2 |
| Adrenal Gland | 15.0 |
| Pituitary gland Pool | 1.8 |
| Salivary Gland | 5.4 |
| Thyroid (female) | 7.0 |
| Pancreatic ca. CAPAN2 | 27.0 |
| Pancreas Pool | 8.7 |

TABLE AAD
Panel 4.1D

| | Rel. Exp. (%) | Rel. Exp. (%) |
|---|---|---|
| | Ag5289, Run | Ag5289, Run |
| Tissue Name | 233229299 | 233232664 |
| Secondary Th1 act | 0.9 | 0.9 |
| Secondary Th2 act | 1.3 | 1.9 |
| Secondary Tr1 act | 0.1 | 0.7 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.5 | 0.9 |
| Primary Tr1 act | 0.3 | 0.6 |
| Primary Th1 rest | 0.0 | 0.0 |
| Primary Th2 rest | 0.0 | 0.1 |
| Primary Tr1 rest | 0.0 | 0.0 |
| CD45RA CD4 | 5.1 | 4.9 |
| lymphocyte act | | |
| CD45RO CD4 | 2.4 | 4.2 |
| lymphocyte act | | |
| CD8 lymphocyte | 0.3 | 0.5 |
| act | | |
| Secondary CD8 | 1.8 | 2.5 |
| lymphocyte rest | | |
| Secondary CD8 | 0.0 | 0.1 |
| lymphocyte act | | |
| CD4 lymphocyte | 0.0 | 0.0 |
| none | | |
| 2ry | 0.0 | 0.0 |
| Th1/Th2/Tr1_anti- | | |
| CD95 CH11 | | |
| LAK cells rest | 0.7 | 0.9 |
| LAK cells IL-2 | 0.6 | 0.9 |
| LAK cells IL- | 0.1 | 0.2 |
| 2 + IL-12 | | |
| LAK cells IL- | 0.5 | 0.9 |
| 2 + IFN gamma | | |
| LAK cells IL-2 + | 0.3 | 0.3 |
| IL-18 | | |
| LAK cells | 2.5 | 4.3 |
| PMA/ionomycin | | |
| NK Cells IL-2 rest | 4.3 | 4.3 |
| Two Way MLR 3 | 0.5 | 0.5 |
| day | | |
| Two Way MLR 5 | 0.1 | 0.0 |
| day | | |
| Two Way MLR 7 | 0.2 | 0.4 |
| day | | |
| PBMC rest | 0.1 | 0.2 |
| PBMC PWM | 0.2 | 0.4 |
| PBMC PHA-L | 1.0 | 1.0 |
| Ramos (B cell) | 1.3 | 2.6 |
| none | | |
| Ramos (B cell) | 26.1 | 29.3 |
| ionomycin | | |
| B lymphocytes | 1.3 | 2.4 |
| PWM | | |
| B lymphocytes | 5.4 | 8.8 |
| CD40L and IL-4 | | |
| EOL-1 dbcAMP | 0.0 | 0.0 |
| EOL-1 dbcAMP | 0.0 | 0.0 |
| PMA/ionomycin | | |
| Dendritic cells | 0.5 | 1.1 |
| none | | |
| Dendritic cells LPS | 0.0 | 0.0 |
| Dendritic cells | 0.1 | 0.3 |
| anti-CD40 | | |
| Monocytes rest | 0.0 | 0.0 |
| Monocytes LPS | 0.3 | 0.7 |
| Macrophages rest | 0.5 | 0.4 |
| Macrophages LPS | 0.5 | 0.9 |
| HUVEC none | 8.5 | 10.5 |
| HUVEC starved | 17.7 | 26.2 |
| HUVEC IL-1beta | 12.2 | 24.0 |
| HUVEC IFN | 12.8 | 16.6 |
| gamma | | |
| HUVEC TNF | 1.3 | 2.0 |
| alpha + IFN | | |
| gamma | | |
| HUVEC TNF | 3.2 | 3.8 |
| alpha + IL4 | | |
| HUVEC IL-11 | 7.4 | 12.7 |
| Lung | 41.2 | 65.5 |
| Microvascular EC | | |
| none | | |
| Lung | 9.9 | 13.5 |
| Microvascular EC | | |
| TNFalpha + IL- | | |
| 1beta | | |
| Microvascular | 0.8 | 1.2 |
| Dermal EC none | | |
| Microsvasular | 3.1 | 4.1 |
| Dermal EC | | |
| TNFalpha + IL- | | |
| 1beta | | |
| Bronchial | 10.6 | 27.4 |
| epithelium | | |
| TNFalpha + | | |
| IL1beta | | |
| Small airway | 7.3 | 13.1 |
| epithelium none | | |
| Small airway | 15.5 | 27.4 |
| epithelium | | |
| TNFalpha + IL- | | |
| 1beta | | |
| Coronery artery | 1.3 | 2.1 |
| SMC rest | | |
| Coronery artery | 1.8 | 2.0 |
| SMC TNFalpha + | | |
| IL-1beta | | |
| Astrocytes rest | 0.1 | 0.1 |
| Astrocytes | 0.1 | 0.2 |
| TNFalpha + IL- | | |
| 1beta | | |
| KU-812 | 6.4 | 11.8 |
| (Basophil) rest | | |
| KU-812 | 20.2 | 35.8 |
| (Basophil) | | |
| PMA/ionomycin | | |
| CCD1106 | 100.0 | 13.4 |
| (Keratinocytes) | | |
| none | | |
| CCD1106 | 8.2 | 14.0 |
| (Keratinocytes) | | |
| TNFalpha + IL- | | |
| 1beta | | |
| Liver cirrhosis | 3.4 | 5.3 |
| NCI-H292 none | 6.7 | 15.3 |
| NCI-H292 IL-4 | 8.8 | 13.1 |
| NCI-H292 IL-9 | 13.7 | 32.1 |
| NCI-H292 IL-13 | 12.4 | 15.6 |
| NCI-H292 IFN | 3.9 | 7.6 |
| gamma | | |
| HPAEC none | 3.4 | 4.6 |
| HPAEC TNF | 11.3 | 16.2 |
| alpha + IL-1 beta | | |
| Lung fibroblast | 1.2 | 1.7 |
| none | | |
| Lung fibroblast | 0.1 | 0.5 |
| TNF alpha + IL-1 | | |
| beta | | |
| Lung fibroblast | 1.9 | 4.2 |
| IL-4 | | |
| Lung fibroblast | 1.7 | 2.1 |
| IL-9 | | |
| Lung fibroblast | 0.2 | 0.5 |
| IL-13 | | |
| Lung fibroblast | 1.9 | 1.9 |
| IFN gamma | | |
| Dermal fibroblast | 3.9 | 6.7 |
| CCD1070 rest | | |
| Dermal fibroblast | 4.4 | 7.5 |
| CCD1070 TNF | | |
| alpha | | |
| Dermal fibroblast | 3.9 | 7.4 |
| CCD1070 IL-1 | | |
| beta | | |
| Dermal fibroblast | 12.2 | 21.9 |
| IFN gamma | | |
| Dermal fibroblast | 72.2 | 100.0 |
| IL-4 | | |
| Dermal | 10.7 | 18.8 |
| Fibroblasts rest | | |
| Neutrophils | 0.1 | 0.2 |
| TNFa + LPS | | |
| Neutrophils rest | 0.1 | 0.0 |
| Colon | 1.6 | 4.0 |
| Lung | 1.0 | 2.2 |
| Thymus | 0.6 | 0.5 |
| Kidney | 4.7 | 6.3 |

CNS_neurodegeneration_v1.0 Summary: Ag5289 This panel does not show differential expression of this gene in Alzheimer's disease. However, this profile confirms the expression of this gene at moderate levels in the brain. Please see Panel 1.5 for discussion of utility of this gene in the central nervous system.

General_screening_panel_v1.5 Summary: Ag5289 Highest expression of this gene is seen in a colon cancer cell line (CT=23.5). This gene is widely expressed in this panel, with high levels of expression seen in brain, colon, gastric, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at high to moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

In addition, this gene is expressed at much higher levels in fetal liver tissue (CT=26.7) when compared to expression in the adult counterpart (CT=30.3). Thus, expression of this gene may be used to differentiate between the fetal and adult source of this tissue.

This gene is also expressed at high levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary: Ag5289 Highest expression is seen in IL-4 treated dermal fibroblasts (CT=26.5). Moderate levels of expression are also seen in clusters of samples derived from lung and dermal fibroblasts, endothelial cells from lung, skin, umbilical vein, and pulmonary artery, small airway and bronchial epithelial cells, and NCI-H292 muco-epidermoid cells. The preponderance of expression in cells derived from the lung and skin suggests that this gene product may be involved in inflammatory processes that involve these organs. Therefore, therapeutic modulation of the expression or function of this gene product may be useful in the treatment of psoriasis, asthma, allergy, and emphysema. A second run with the same probe and primer set, run 233229299, is not included because the amp plot indicates there were experimental difficulties with this run.

AB. CG157486-01: Ephrin Receptor A2.

Expression of gene CG157486-01 was assessed using the primer-probe set Ag2620, described in Table ABA. Results of the RTQ-PCR runs are shown in Tables ABB, ABC, ABD, ABE and ABF.

TABLE ABA
Probe Name Ag2620

| | | | Start | SEQ ID |
|---|---|---|---|---|
| Primers | Sequence | Length | Position | No |
| Forward | 5′-gaagtggtactgctggactttg-3′ | 22 | 195 | 552 |
| Probe | TET-5′-ctcacacacccgtatggcaaagggt-3′-TAMRA | 25 | 243 | 553 |
| Reverse | 5′-cattcatgatgttctgcatcag-3′ | 22 | 273 | 554 |

TABLE ABB
General_screening_panel_v1.5

| | Rel. Exp. (%) |
|---|---|
| | Ag2620, Run |
| Tissue Name | 229827540 |
| Adipose | 2.4 |
| Melanoma* Hs688(A).T | 6.1 |
| Melanoma* Hs688(B).T | 7.7 |
| Melanoma* M14 | 0.7 |
| Melanoma* LOXIMVI | 12.7 |
| Melanoma* SK-MEL-5 | 1.8 |
| Squamous cell carcinoma SCC-4 | 11.0 |
| Testis Pool | 0.4 |
| Prostate ca.* (bone met) PC-3 | 100.0 |
| Prostate Pool | 0.7 |
| Placenta | 2.4 |
| Uterus Pool | 1.8 |
| Ovarian ca. OVCAR-3 | 25.5 |
| Ovarian ca. SK-OV-3 | 64.6 |
| Ovarian ca. OVCAR-4 | 17.0 |
| Ovarian ca. OVCAR-5 | 37.4 |
| Ovarian ca. IGROV-1 | 41.8 |
| Ovarian ca. OVCAR-8 | 18.6 |
| Ovary | 1.2 |
| Breast ca. MCF-7 | 2.5 |
| Breast ca. MDA-MB-231 | 57.4 |
| Breast ca. BT 549 | 22.8 |
| Breast ca. T47D | 0.2 |
| Breast ca. MDA-N | 0.9 |
| Breast Pool | 1.5 |
| Trachea | 4.2 |
| Lung | 0.0 |
| Fetal Lung | 7.3 |
| Lung ca. NCI-N417 | 0.7 |
| Lung ca. LX-1 | 40.3 |
| Lung ca. NCI-H146 | 0.1 |
| Lung ca. SHP-77 | 0.3 |
| Lung ca. A549 | 9.6 |
| Lung ca. NCI-H526 | 0.4 |
| Lung ca. NCI-H23 | 4.8 |
| Lung ca. NCI-H460 | 5.2 |
| Lung ca. HOP-62 | 22.4 |
| Lung ca. NCI-H522 | 12.0 |
| Liver | 0.4 |
| Fetal Liver | 1.1 |
| Liver ca. HepG2 | 19.2 |
| Kidney Pool | 5.1 |
| Fetal Kidney | 1.2 |
| Renal ca. 786-0 | 18.0 |
| Renal ca. A498 | 3.5 |
| Renal ca. ACHN | 12.3 |
| Renal ca. UO-31 | 22.5 |
| Renal ca. TK-10 | 29.1 |
| Bladder | 3.7 |
| Gastric ca. (liver met.) NCI-N87 | 69.7 |
| Gastric ca. KATO III | 69.3 |
| Colon ca. SW-948 | 23.8 |
| Colon ca. SW480 | 36.9 |
| Colon ca.* (SW480 met) SW620 | 22.5 |
| Colon ca. HT29 | 7.9 |
| Colon ca. HCT-116 | 30.8 |
| Colon ca. CaCo-2 | 6.1 |
| Colon cancer tissue | 13.8 |
| Colon ca. SW1116 | 4.2 |
| Colon ca. Colo-205 | 1.7 |
| Colon ca. SW-48 | 5.3 |
| Colon Pool | 2.6 |
| Small Intestine Pool | 1.4 |
| Stomach Pool | 1.9 |
| Bone Marrow Pool | 0.4 |
| Fetal Heart | 0.7 |
| Heart Pool | 1.1 |
| Lymph Node Pool | 1.2 |
| Fetal Skeletal Muscle | 0.3 |
| Skeletal Muscle Pool | 1.1 |
| Spleen Pool | 2.1 |
| Thymus Pool | 0.9 |
| CNS cancer (glio/astro) U87-MG | 1.0 |
| CNS cancer (glio/astro) U-118-MG | 19.5 |
| CNS cancer (neuro; met) SK-N-AS | 7.2 |
| CNS cancer (astro) SF-539 | 12.3 |
| CNS cancer (astro) SNB-75 | 23.2 |
| CNS cancer (glio) SNB-19 | 41.8 |
| CNS cancer (glio) SF-295 | 42.9 |
| Brain (Amygdala) Pool | 0.1 |
| Brain (cerebellum) | 0.3 |
| Brain (fetal) | 0.5 |
| Brain (Hippocampus) Pool | 0.2 |
| Cerebral Cortex Pool | 0.1 |
| Brain (Substantia nigra) Pool | 0.4 |
| Brain (Thalamus) Pool | 0.3 |
| Brain (whole) | 0.2 |
| Spinal Cord Pool | 0.4 |
| Adrenal Gland | 1.4 |
| Pituitary gland Pool | 0.1 |
| Salivary Gland | 7.1 |
| Thyroid (female) | 2.7 |
| Pancreatic ca. CAPAN2 | 59.5 |
| Pancreas Pool | 2.1 |

TABLE ABC
Oncology_cell_line_screening_panel_v3.1

| | Rel. Exp. (%) |
|---|---|
| | Ag2620, Run |
| Tissue Name | 230277126 |
| Daoy Medulloblastoma/Cerebellum | 1.5 |
| TE671 Medulloblastom/Cerebellum | 3.1 |
| D283 Med Medulloblastoma/Cerebellum | 24.5 |
| PFSK-1 Primitive | 19.3 |
| Neuroectodermal/Cerebellum | |
| XF-498_CNS | 23.5 |
| SNB-78_CNS/glioma | 5.5 |
| SF-268_CNS/glioblastoma | 29.3 |
| T98G_Glioblastoma | 13.6 |
| SK-N-SH_Neuroblastoma (metastasis) | 6.5 |
| SF-295_CNS/glioblastoma | 17.3 |
| Cerebellum | 0.1 |
| Cerebellum | 0.0 |
| NCI-H292_Mucoepidermoid lung ca. | 83.5 |
| DMS-114_Small cell lung cancer | 3.3 |
| DMS-79_Small cell lung | 0.9 |
| cancer/neuroendocrine | |
| NCI-H146_Small cell lung | 0.4 |
| cancer/neuroendocrine | |
| NCI-H526_Small cell lung | 1.0 |
| cancer/neuroendocrine | |
| NCI-N417_Small cell lung | 0.6 |
| cancer/neuroendocrine | |
| NCI-H82_Small cell lung | 0.7 |
| cancer/neuroendocrine | |
| NCI-H157_Squamous cell lung cancer | 14.0 |
| (metastasis) | |
| NCI-H1155_Large cell lung | 0.1 |
| cancer/neuroendocrine | |
| NCI-H1299_Large cell lung | 21.9 |
| cancer/neuroendocrine | |
| NCI-H727_Lung carcinoid | 14.5 |
| NCI-UMC-11_Lung carcinoid | 0.0 |
| LX-1_Small cell lung cancer | 20.3 |
| Colo-205_Colon cancer | 1.9 |
| KM12_Colon cancer | 16.3 |
| KM20L2_Colon cancer | 9.5 |
| NCI-H716_Colon cancer | 15.1 |
| SW-48_Colon adenocarcinoma | 5.2 |
| SW1116_Colon adenocarcinoma | 5.0 |
| LS 174T_Colon adenocarcinoma | 25.2 |
| SW-948_Colon adenocarcinoma | 1.4 |
| SW-480_Colon adenocarcinoma | 3.3 |
| NCI-SNU-5_Gastric ca. | 14.7 |
| KATO III_Stomach | 20.7 |
| NCI-SNU-16_Gastric ca. | 8.8 |
| NCI-SNU-1_Gastric ca. | 6.1 |
| RF-1_Gastric adenocarcinoma | 0.1 |
| RF-48_Gastric adenocarcinoma | 0.1 |
| MKN-45_Gastric ca. | 27.5 |
| NCI-N87_Gastric ca. | 20.0 |
| OVCAR-5_Ovarian ca. | 16.2 |
| RL95-2_Uterine carcinoma | 4.2 |
| HelaS3_Cervical adenocarcinoma | 9.0 |
| Ca Ski Cervical epidermoid carcinoma | 58.2 |
| (metastasis) | |
| ES-2_Ovarian clear cell carcinoma | 15.8 |
| Ramos/6 h stim_Stimulated with | 0.0 |
| PMA/ionomycin 6 h | |
| Ramos/14 h stim_Stimulated with | 0.0 |
| PMA/ionomycin 14 h | |
| MEG-01_Chronic myelogenous leukemia | 0.2 |
| (megokaryoblast) | |
| Raji_Burkitt's lymphoma | 0.1 |
| Daudi_Burkitt's lymphoma | 0.0 |
| U266_B-cell plasmacytoma/myeloma | 0.0 |
| CA46_Burkitt's lymphoma | 0.0 |
| RL_non-Hodgkin's B-cell lymphoma | 0.0 |
| JM1_pre-B-cell lymphoma/leukemia | 0.0 |
| Jurkat_T cell leukemia | 0.0 |
| TF-1_Erythroleukemia | 0.1 |
| HUT 78_T-cell lymphoma | 0.7 |
| U937_Histiocytic lymphoma | 0.0 |
| KU-812_Myelogenous leukemia | 0.0 |
| 769-P_Clear cell renal ca. | 9.3 |
| Caki-2_Clear cell renal ca. | 9.9 |
| SW 839_Clear cell renal ca. | 31.2 |
| G401_Wilms' tumor | 4.6 |
| Hs766T_Pancreatic ca. (LN metastasis) | 100.0 |
| CAPAN-1_Pancreatic adenocarcinoma | 50.0 |
| (liver metastasis) | |
| SU86.86_Pancreatic carcinoma | 64.2 |
| (liver metastasis) | |
| BxPC-3_Pancreatic adenocarcinoma | 35.1 |
| HPAC_Pancreatic adenocarcinoma | 58.6 |
| MIA PaCa-2_Pancreatic ca. | 18.3 |
| CFPAC-1_Pancreatic ductal | 73.7 |
| adenocarcinoma | |
| PANC-1_Pancreatic epithelioid | 70.2 |
| ductal ca. | |
| T24_Bladder ca. (transitional cell) | 16.5 |
| 5637_Bladder ca. | 35.8 |
| HT-1197_Bladder ca. | 35.1 |
| UM-UC-3_Bladder ca. (transitional cell) | 9.3 |
| A204_Rhabdomyosarcoma | 6.7 |
| HT-1080_Fibrosarcoma | 18.0 |
| MG-63_Osteosarcoma (bone) | 11.3 |
| SK-LMS-1_Leiomyosarcoma (vulva) | 12.9 |
| SJRH30_Rhabdomyosarcoma (met to bone | 12.2 |
| marrow) | |
| A431_Epidermoid ca. | 36.6 |
| WM266-4_Melanoma | 0.3 |
| DU 145_Prostate | 12.3 |
| MDA-MB-468_Breast adenocarcinoma | 2.7 |
| SSC-4_Tongue | 7.5 |
| SSC-9_Tongue | 12.2 |
| SSC-15_Tongue | 9.3 |
| CAL 27_Squamous cell ca. of tongue | 17.0 |

TABLE ABD
Panel 1.3D

| | Rel. Exp. (%) |
|---|---|
| | Ag2620, Run |
| Tissue Name | 167660097 |
| Liver adenocarcinoma | 52.9 |
| Pancreas | 2.6 |
| Pancreatic ca. CAPAN2 | 33.0 |
| Adrenal gland | 0.9 |
| Thyroid | 0.6 |
| Salivary gland | 8.8 |
| Pituitary gland | 0.5 |
| Brain (fetal) | 1.7 |
| Brain (whole) | 0.3 |
| Brain (amygdala) | 0.7 |
| Brain (cerebellum) | 0.0 |
| Brain (hippocampus) | 1.0 |
| Brain (Substantia nigra) | 0.9 |
| Brain (thalamus) | 0.6 |
| Cerebral Cortex | 0.4 |
| Spinal cord | 1.5 |
| glio/astro U87-MG | 1.3 |
| glio/astro U-118-MG | 14.1 |
| astrocytoma SW1783 | 25.5 |
| neuro*; met SK-N-AS | 3.7 |
| astrocytoma SF-539 | 9.0 |
| astrocytoma SNB-75 | 21.3 |
| glioma SNB-19 | 21.0 |
| glioma U251 | 35.1 |
| glioma SF-295 | 31.6 |
| Heart (fetal) | 16.6 |
| Heart | 1.2 |
| Skeletal muscle (fetal) | 2.7 |
| Skeletal muscle | 0.7 |
| Bone marrow | 0.3 |
| Thymus | 1.0 |
| Spleen | 1.5 |
| Lymph node | 4.2 |
| Colorectal | 4.4 |
| Stomach | 1.0 |
| Small intestine | 1.6 |
| Colon ca. SW480 | 27.2 |
| Colon ca.* SW620(SW480 met) | 39.8 |
| Colon ca. HT29 | 9.5 |
| Colon ca. HCT-116 | 14.0 |
| Colon ca. CaCo-2 | 7.1 |
| Colon ca. tissue(ODO3866) | 13.3 |
| Colon ca. HCC-2998 | 49.7 |
| Gastric ca.* (liver met) NCI-N87 | 48.3 |
| Bladder | 1.9 |
| Trachea | 4.3 |
| Kidney | 3.3 |
| Kidney (fetal) | 26.6 |
| Renal ca. 786-0 | 21.0 |
| Renal ca. A498 | 30.6 |
| Renal ca. RXF 393 | 29.3 |
| Renal ca. ACHN | 25.0 |
| Renal ca. UO-31 | 17.2 |
| Renal ca. TK-10 | 20.7 |
| Liver | 0.7 |
| Liver (fetal) | 3.5 |
| Liver ca. (hepatoblast) HepG2 | 17.4 |
| Lung | 3.3 |
| Lung (fetal) | 3.0 |
| Lung ca. (small cell) LX-1 | 21.6 |
| Lung ca. (small cell) NCI-H69 | 0.0 |
| Lung ca. (s. cell var.) SHP-77 | 0.9 |
| Lung ca. (large cell)NCI-H460 | 1.8 |
| Lung ca. (non-sm. cell) A549 | 8.8 |
| Lung ca. (non-s. cell) NCI-H23 | 3.9 |
| Lung ca. (non-s. cell) HOP-62 | 28.3 |
| Lung ca. (non-s. cl) NCI-H522 | 16.7 |
| Lung ca. (squam.) SW 900 | 15.5 |
| Lung ca. (squam.) NCI-H596 | 0.2 |
| Mammary gland | 5.1 |
| Breast ca.* (pl. ef) MCF-7 | 1.5 |
| Breast ca.* (pl. ef) MDA-MB-231 | 41.8 |
| Breast ca.* (pl. ef) T47D | 0.5 |
| Breast ca. BT-549 | 28.7 |
| Breast ca. MDA-N | 1.1 |
| Ovary | 2.3 |
| Ovarian ca. OVCAR-3 | 33.0 |
| Ovarian ca. OVCAR-4 | 18.9 |
| Ovarian ca. OVCAR-5 | 92.0 |
| Ovarian ca. OVCAR-8 | 3.4 |
| Ovarian ca. IGROV-1 | 5.0 |
| Ovarian ca.* (ascites) SK-OV-3 | 100.0 |
| Uterus | 2.1 |
| Placenta | 2.4 |
| Prostate | 1.2 |
| Prostate ca.* (bone met)PC-3 | 64.6 |
| Testis | 0.4 |
| Melanoma Hs688(A).T | 4.1 |
| Melanoma* (met) Hs688(B).T | 3.9 |
| Melanoma UACC-62 | 6.3 |
| Melanoma M14 | 0.0 |
| Melanoma LOXIMVI | 14.0 |
| Melanoma* (met) SK-MEL-5 | 0.9 |
| Adipose | 7.0 |

TABLE ABE
Panel 2.2

| | Rel. Exp. (%) |
|---|---|
| | Ag2620, Run |
| Tissue Name | 175135887 |
| Normal Colon | 6.9 |
| Colon cancer (OD06064) | 34.9 |
| Colon Margin (OD06064) | 3.7 |
| Colon cancer (OD06159) | 18.9 |
| Colon Margin (OD06159) | 1.9 |
| Colon cancer (OD06297-04) | 9.3 |
| Colon Margin (OD06297-05) | 14.5 |
| CC Gr.2 ascend colon (ODO3921) | 38.2 |
| CC Margin (ODO3921) | 8.8 |
| Colon cancer metastasis (OD06104) | 1.7 |
| Lung Margin (OD06104) | 3.0 |
| Colon mets to lung (OD04451-01) | 28.9 |
| Lung Margin (OD04451-02) | 6.3 |
| Normal Prostate | 3.0 |
| Prostate Cancer (OD04410) | 1.4 |
| Prostate Margin (OD04410) | 1.6 |
| Normal Ovary | 12.1 |
| Ovarian cancer (OD06283-03) | 2.7 |
| Ovarian Margin (OD06283-07) | 5.5 |
| Ovarian Cancer 064008 | 16.3 |
| Ovarian cancer (OD06145) | 10.4 |
| Ovarian Margin (OD06145) | 8.4 |
| Ovarian cancer (OD06455-03) | 22.7 |
| Ovarian Margin (OD06455-07) | 2.8 |
| Normal Lung | 7.0 |
| Invasive poor diff. lung adeno | 1.6 |
| (ODO4945-01 | |
| Lung Margin (ODO4945-03) | 25.3 |
| Lung Malignant Cancer (OD03126) | 3.3 |
| Lung Margin (OD03126) | 16.2 |
| Lung Cancer (OD05014A) | 22.4 |
| Lung Margin (OD05014B) | 15.5 |
| Lung cancer (OD06081) | 5.6 |
| Lung Margin (OD06081) | 2.9 |
| Lung Cancer (OD04237-01) | 13.3 |
| Lung Margin (OD04237-02) | 37.1 |
| Ocular Melanoma Metastasis | 11.3 |
| Ocular Melanoma Margin (Liver) | 35.8 |
| Melanoma Metastasis | 7.3 |
| Melanoma Margin (Lung) | 7.5 |
| Normal Kidney | 4.0 |
| Kidney Ca, Nuclear grade 2 (OD04338) | 39.2 |
| Kidney Margin (OD04338) | 6.4 |
| Kidney Ca Nuclear grade 1/2 | 39.0 |
| (OD04339) | |
| Kidney Margin (OD04339) | 3.8 |
| Kidney Ca, Clear cell type (OD04340) | 51.1 |
| Kidney Margin (OD04340) | 16.8 |
| Kidney Ca, Nuclear grade 3 (OD04348) | 4.9 |
| Kidney Margin (OD04348) | 100.0 |
| Kidney malignant cancer (OD06204B) | 11.6 |
| Kidney normal adjacent tissue | 3.4 |
| (OD06204E) | |
| Kidney Cancer (OD04450-01) | 87.7 |
| Kidney Margin (OD04450-03) | 5.1 |
| Kidney Cancer 8120613 | 0.0 |
| Kidney Margin 8120614 | 5.4 |
| Kidney Cancer 9010320 | 17.6 |
| Kidney Margin 9010321 | 8.2 |
| Kidney Cancer 8120607 | 42.3 |
| Kidney Margin 8120608 | 18.7 |
| Normal Uterus | 11.0 |
| Uterine Cancer 064011 | 11.5 |
| Normal Thyroid | 2.0 |
| Thyroid Cancer 064010 | 46.3 |
| Thyroid Cancer A302152 | 20.2 |
| Thyroid Margin A302153 | 9.9 |
| Normal Breast | 12.9 |
| Breast Cancer (OD04566) | 1.2 |
| Breast Cancer 1024 | 5.8 |
| Breast Cancer (OD04590-01) | 0.2 |
| Breast Cancer Mets (OD04590-03) | 2.4 |
| Breast Cancer Metastasis | 16.3 |
| (OD04655-05) | |
| Breast Cancer 064006 | 1.6 |
| Breast Cancer 9100266 | 5.2 |
| Breast Margin 9100265 | 2.5 |
| Breast Cancer A209073 | 4.5 |
| Breast Margin A2090734 | 14.3 |
| Breast cancer (OD06083) | 3.9 |
| Breast cancer node metastasis | 2.2 |
| (OD06083) | |
| Normal Liver | 7.9 |
| Liver Cancer 1026 | 19.3 |
| Liver Cancer 1025 | 18.2 |
| Liver Cancer 6004-T | 12.9 |
| Liver Tissue 6004-N | 3.7 |
| Liver Cancer 6005-T | 11.3 |
| Liver Tissue 6005-N | 28.1 |
| Liver Cancer 064003 | 12.4 |
| Normal Bladder | 18.0 |
| Bladder Cancer 1023 | 11.7 |
| Bladder Cancer A302173 | 5.6 |
| Normal Stomach | 39.5 |
| Gastric Cancer 9060397 | 24.5 |
| Stomach Margin 9060396 | 28.3 |
| Gastric Cancer 9060395 | 10.0 |
| Stomach Margin 9060394 | 29.9 |
| Gastric Cancer 064005 | 25.2 |

TABLE ABF
general oncology screening panel_v_2.4

| | Rel. Exp. (%) |
|---|---|
| | Ag2620, Run |
| Tissue Name | 259737766 |
| Colon cancer 1 | 67.8 |
| Colon cancer NAT 1 | 17.2 |
| Colon cancer 2 | 48.6 |
| Colon cancer NAT 2 | 5.7 |
| Colon cancer 3 | 49.0 |
| Colon cancer NAT 3 | 27.5 |
| Colon malignant cancer 4 | 95.3 |
| Colon normal adjacent tissue 4 | 5.8 |
| Lung cancer 1 | 14.7 |
| Lung NAT 1 | 0.7 |
| Lung cancer 2 | 100.0 |
| Lung NAT 2 | 3.1 |
| Squamous cell carcinoma 3 | 18.7 |
| Lung NAT 3 | 1.8 |
| metastatic melanoma 1 | 5.6 |
| Melanoma 2 | 11.8 |
| Melanoma 3 | 5.8 |
| metastatic melanoma 4 | 12.2 |
| metastatic melanoma 5 | 17.1 |
| Bladder cancer 1 | 0.6 |
| Bladder cancer NAT 1 | 0.0 |
| Bladder cancer 2 | 10.0 |
| Bladder cancer NAT 2 | 0.0 |
| Bladder cancer NAT 3 | 1.8 |
| Bladder cancer NAT 4 | 2.8 |
| Prostate adenocarcinoma 1 | 4.6 |
| Prostate adenocarcinoma 2 | 3.4 |
| Prostate adenocarcinoma 3 | 5.4 |
| Prostate adenocarcinoma 4 | 93.3 |
| Prostate cancer NAT 5 | 4.1 |
| Prostate adenocarcinoma 6 | 0.8 |
| Prostate adenocarcinoma 7 | 3.0 |
| Prostate adenocarcinoma 8 | 1.0 |
| Prostate adenocarcinoma 9 | 5.4 |
| Prostate cancer NAT 10 | 1.8 |
| Kidney cancer 1 | 13.6 |
| Kidney NAT 1 | 8.0 |
| Kidney cancer 2 | 24.5 |
| Kidney NAT 2 | 13.9 |
| Kidney cancer 3 | 38.7 |
| Kidney NAT 3 | 8.1 |
| Kidney cancer 4 | 26.6 |
| Kidney NAT 4 | 15.0 |

General_screening_panel_v1.5 Summary: Ag2620 Highest expression of this gene is seen in a prostate cancer cell line (CT=25.9). In addition, high to moderate levels of expression are seen in all the clusters of cancer cell line samples on this panel, including brain, colon, gastric, pancreatic, renal, lung, breast, ovarian, and melanoma cancer cell lines. This expression profile suggests a role for this gene product in cell survival and proliferation. Modulation of this gene product may be useful in the treatment of cancer.

This gene encodes an ephrin receptor A2-like protein (EphA2) which is activated by phosphorylation both in the tumor itself and the endothelial cells associated with the tumor. This activation is especially prominent in tumor types that are highly vascularized like colon, kidney and ovarian cancers. It appears that without the proper ligand, this overexpression and activation leads to cell transformation and the promotion of tumor-related angiogenesis which affect the overall balance between survival/apoptotic stimuli. Modications in the signaling emanating from this receptor will impact that balance resulting either in increased survival (stimulation of angiogenesis) or increased apoptosis (inhibition of tumorogenesis both directly against tumor cells and indirectly against endothelial cells. Therefore, therapeutic targeting of this gene product with a human monoclonal antibody will affect the overall balance between survival/apoptotic stimuli in cell expressing it, preferably endothelial, tumor and neuronal cells and will therefore affect the outcome of diseases where these stimuli are involved in the pathogenesis, tumors, preferably colon, kidney and ovarian cancer, pathogenic angiogenesis, preferably wound healing, neurodegenaritive diseases.

Among tissues with metabolic function, this gene is expressed at moderate to low levels in adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

This gene is also expressed at low but significant levels in the CNS, including the hippocampus, thalamus, substantia nigra, amygdala, and cerebellum. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Oncology_cell_line_screening_panel_v3.1 Summary: Ag2620 Highest expression is seen in a pancreatic cancer cell line (CT=27.8). Moderate levels of expression are also seen in many of the cell lines on this panel. Please see Panel 1.5 for discussion of utility of this gene in the treatment of cancer.

Panel 1.3D Summary: Ag2620 Highest expression of this gene is seen in an ovarian cancer cell line (CT=29.3). In addition, moderate to low levels of expression are seen in many of the clusters of cancer cell line samples on this panel, including brain, colon, gastric, pancreatic, renal, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at low levels in adipose, pancreas, and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

In addition, this gene is expressed at much higher levels in fetal heart tissue (CT=32) when compared to expression in the adult counterpart (CT=35). Thus, expression of this gene may be used to differentiate between the fetal and adult source of this tissue.

Panel 2.2 Summary: Ag2620 Highest expression is seen in a sample of normal kidney (CT=3 1). In addition, this gene appears to be more highly expressed in kidney cancer than in the corresponding normal adjacent tissue. Thus, expression of this gene could be used as a marker of this cancer. Furthemore, therapeutic modulation of the expression or function of this gene product may be useful in the treatment of kidney cancer.

general oncology screening panel_v_{—}2.4 Summary: Ag2620 Highest expression is seen in a sample of lung cancer (CT=29.5). In addition, this gene appears to be more highly expressed in colon and kidney cancers than in the corresponding normal adjacent tissue. Thus, expression of this gene could be used as a marker of these cancers. Furthemore, therapeutic modulation of the expression or function of this gene product may be useful in the treatment of colon and kidney cancer.

AC. CG157505-01: Kinesin 16A.

Expression of gene CG157505-01 was assessed using the primer-probe set Ag5721, described in Table ACA. Results of the RTQ-PCR runs are shown in Tables ACB, ACC and ACD.

TABLE ACA
Probe Name Ag5721

| | | | Start | SEQ ID |
|---|---|---|---|---|
| Primers | Sequence | Length | Position | No |
| Forward | 5′-ctgaaggagccaatatcaacaa-3′ | 22 | 809 | 555 |
| Probe | TET-5′-tcccttgtgactctaggaattgtcatctcc-3′-TAMRA | 30 | 832 | 556 |
| Reverse | 5′-gctgaaaacttgggagttctg-3′ | 21 | 871 | 557 |

TABLE ACB
CNS_neurodegeneration_v1.0

| | Rel. Exp. (%) |
|---|---|
| | Ag5721, Run |
| Tissue Name | 247018773 |
| AD 1 Hippo | 18.0 |
| AD 2 Hippo | 16.8 |
| AD 3 Hippo | 10.1 |
| AD 4 Hippo | 7.0 |
| AD 5 hippo | 87.7 |
| AD 6 Hippo | 27.0 |
| Control 2 Hippo | 21.0 |
| Control 4 Hippo | 11.7 |
| Control (Path) 3 Hippo | 5.8 |
| AD 1 Temporal Ctx | 40.9 |
| AD 2 Temporal Ctx | 25.5 |
| AD 3 Temporal Ctx | 5.7 |
| AD 4 Temporal Ctx | 24.3 |
| AD 5 Inf Temporal Ctx | 100.0 |
| AD 5 Sup Temporal Ctx | 52.5 |
| AD 6 Inf Temporal Ctx | 72.7 |
| AD 6 Sup Temporal Ctx | 44.4 |
| Control 1 Temporal Ctx | 9.0 |
| Control 2 Temporal Ctx | 17.6 |
| Control 3 Temporal Ctx | 16.8 |
| Control 4 Temporal Ctx | 11.7 |
| Control (Path) 1 Temporal Ctx | 36.1 |
| Control (Path) 2 Temporal Ctx | 27.0 |
| Control (Path) 3 Temporal Ctx | 4.9 |
| Control (Path) 4 Temporal Ctx | 20.4 |
| AD 1 Occipital Ctx | 24.8 |
| AD 2 Occipital Ctx (Missing) | 0.0 |
| AD 3 Occipital Ctx | 9.6 |
| AD 4 Occipital Ctx | 21.9 |
| AD 5 Occipital Ctx | 25.5 |
| AD 6 Occipital Ctx | 24.8 |
| Control 1 Occipital Ctx | 5.1 |
| Control 2 Occipital Ctx | 43.2 |
| Control 3 Occipital Ctx | 26.1 |
| Control 4 Occipital Ctx | 10.3 |
| Control (Path) 1 Occipital Ctx | 72.2 |
| Control (Path) 2 Occipital Ctx | 13.9 |
| Control (Path) 3 Occipital Ctx | 3.5 |
| Control (Path) 4 Occipital Ctx | 23.7 |
| Control 1 Parietal Ctx | 8.1 |
| Control 2 Parietal Ctx | 65.5 |
| Control 3 Parietal Ctx | 18.0 |
| Control (Path) 1 Parietal Ctx | 34.9 |
| Control (Path) 2 Parietal Ctx | 26.8 |
| Control (Path) 3 Parietal Ctx | 2.1 |
| Control (Path) 4 Parietal Ctx | 39.2 |

TABLE ACC
General_screening_panel_v1.5

| | Rel. Exp. (%) |
|---|---|
| | Ag5721, Run |
| Tissue Name | 245454345 |
| Adipose | 11.0 |
| Melanoma* Hs688(A).T | 5.4 |
| Melanoma* Hs688(B).T | 2.0 |
| Melanoma* M14 | 13.2 |
| Melanoma* LOXIMVI | 7.6 |
| Melanoma* SK-MEL-5 | 4.6 |
| Squamous cell carcinoma SCC-4 | 1.0 |
| Testis Pool | 28.3 |
| Prostate ca.* (bone met) PC-3 | 6.4 |
| Prostate Pool | 10.6 |
| Placenta | 9.7 |
| Uterus Pool | 48.0 |
| Ovarian ca. OVCAR-3 | 3.6 |
| Ovarian ca. SK-OV-3 | 19.1 |
| Ovarian ca. OVCAR-4 | 1.4 |
| Ovarian ca. OVCAR-5 | 6.1 |
| Ovarian ca. IGROV-1 | 5.4 |
| Ovarian ca. OVCAR-8 | 7.1 |
| Ovary | 29.5 |
| Breast ca. MCF-7 | 1.0 |
| Breast ca. MDA-MB-231 | 15.2 |
| Breast ca. BT 549 | 28.9 |
| Breast ca. T47D | 0.3 |
| Breast ca. MDA-N | 3.2 |
| Breast Pool | 38.2 |
| Trachea | 21.9 |
| Lung | 8.4 |
| Fetal Lung | 100.0 |
| Lung ca. NCI-N417 | 2.9 |
| Lung ca. LX-1 | 5.2 |
| Lung ca. NCI-H146 | 5.5 |
| Lung ca. SHP-77 | 8.8 |
| Lung ca. A549 | 7.2 |
| Lung ca. NCI-H526 | 1.1 |
| Lung ca. NCI-H23 | 15.0 |
| Lung ca. NCI-H460 | 4.0 |
| Lung ca. HOP-62 | 12.2 |
| Lung ca. NCI-H522 | 20.9 |
| Liver | 0.3 |
| Fetal Liver | 3.3 |
| Liver ca. HepG2 | 13.0 |
| Kidney Pool | 71.2 |
| Fetal Kidney | 19.8 |
| Renal ca. 786-0 | 11.1 |
| Renal ca. A498 | 3.1 |
| Renal ca. ACHN | 13.7 |
| Renal ca. UO-31 | 5.6 |
| Renal ca. TK-10 | 18.9 |
| Bladder | 6.0 |
| Gastric ca. (liver met.) NCI-N87 | 1.6 |
| Gastric ca. KATO III | 0.5 |
| Colon ca. SW-948 | 0.5 |
| Colon ca. SW480 | 8.3 |
| Colon ca.* (SW480 met) SW620 | 6.5 |
| Colon ca. HT29 | 0.1 |
| Colon ca. HCT-116 | 16.3 |
| Colon ca. CaCo-2 | 1.2 |
| Colon cancer tissue | 5.5 |
| Colon ca. SW1116 | 1.7 |
| Colon ca. Colo-205 | 0.0 |
| Colon ca. SW-48 | 0.0 |
| Colon Pool | 43.5 |
| Small Intestine Pool | 32.5 |
| Stomach Pool | 19.2 |
| Bone Marrow Pool | 16.6 |
| Fetal Heart | 38.4 |
| Heart Pool | 15.7 |
| Lymph Node Pool | 35.4 |
| Fetal Skeletal Muscle | 24.0 |
| Skeletal Muscle Pool | 13.7 |
| Spleen Pool | 16.4 |
| Thymus Pool | 31.6 |
| CNS cancer (glio/astro) U87-MG | 17.7 |
| CNS cancer (glio/astro) U-118-MG | 16.6 |
| CNS cancer (neuro; met) SK-N-AS | 18.9 |
| CNS cancer (astro) SF-539 | 15.9 |
| CNS cancer (astro) SNB-75 | 24.8 |
| CNS cancer (glio) SNB-19 | 6.3 |
| CNS cancer (glio) SF-295 | 19.6 |
| Brain (Amygdala) Pool | 11.0 |
| Brain (cerebellum) | 31.2 |
| Brain (fetal) | 28.1 |
| Brain (Hippocampus) Pool | 6.6 |
| Cerebral Cortex Pool | 10.5 |
| Brain (Substantia nigra) Pool | 10.3 |
| Brain (Thalamus) Pool | 15.5 |
| Brain (whole) | 7.7 |
| Spinal Cord Pool | 13.5 |
| Adrenal Gland | 6.2 |
| Pituitary gland Pool | 1.2 |
| Salivary Gland | 2.3 |
| Thyroid (female) | 2.0 |
| Pancreatic ca. CAPAN2 | 0.2 |
| Pancreas Pool | 26.1 |

TABLE ACD
Panel 4.1D

| | Rel. Exp. (%) |
|---|---|
| | Ag5721, Run |
| Tissue Name | 246509239 |
| Secondary Th1 act | 36.3 |
| Secondary Th2 act | 22.8 |
| Secondary Tr1 act | 5.3 |
| Secondary Th1 rest | 2.6 |
| Secondary Th2 rest | 0.0 |
| Secondary Tr1 rest | 2.1 |
| Primary Th1 act | 0.0 |
| Primary Th2 act | 17.7 |
| Primary Tr1 act | 11.9 |
| Primary Th1 rest | 0.4 |
| Primary Th2 rest | 5.1 |
| Primary Tr1 rest | 1.1 |
| CD45RA CD4 lymphocyte act | 17.2 |
| CD45RO CD4 lymphocyte act | 23.8 |
| CD8 lymphocyte act | 2.5 |
| Secondary CD8 lymphocyte rest | 14.9 |
| Secondary CD8 lymphocyte act | 1.5 |
| CD4 lymphocyte none | 0.7 |
| 2ry Th1/Th2/Tr1_anti-CD95 CH11 | 5.8 |
| LAK cells rest | 3.2 |
| LAK cells IL-2 | 2.7 |
| LAK cells IL-2 + IL-12 | 0.0 |
| LAK cells IL-2 + IFN gamma | 4.9 |
| LAK cells IL-2 + IL-18 | 1.3 |
| LAK cells PMA/ionomycin | 3.5 |
| NK Cells IL-2 rest | 94.6 |
| Two Way MLR 3 day | 4.5 |
| Two Way MLR 5 day | 1.5 |
| Two Way MLR 7 day | 2.3 |
| PBMC rest | 1.5 |
| PBMC PWM | 1.8 |
| PBMC PHA-L | 3.6 |
| Ramos (B cell) none | 4.7 |
| Ramos (B cell) ionomycin | 26.4 |
| B lymphocytes PWM | 4.9 |
| B lymphocytes CD40L and IL-4 | 13.7 |
| EOL-1 dbcAMP | 14.7 |
| EOL-1 dbcAMP PMA/ionomycin | 0.6 |
| Dendritic cells none | 8.7 |
| Dendritic cells LPS | 0.7 |
| Dendritic cells anti-CD40 | 0.6 |
| Monocytes rest | 0.0 |
| Monocytes LPS | 2.0 |
| Macrophages rest | 1.5 |
| Macrophages LPS | 0.0 |
| HUVEC none | 9.3 |
| HUVEC starved | 13.3 |
| HUVEC IL-1beta | 13.1 |
| HUVEC IFN gamma | 26.2 |
| HUVEC TNF alpha + IFN gamma | 0.5 |
| HUVEC TNF alpha + IL4 | 2.7 |
| HUVEC IL-11 | 14.9 |
| Lung Microvascular EC none | 40.3 |
| Lung Microvascular EC TNFalpha + | 14.6 |
| IL-1beta | |
| Microvascular Dermal EC none | 4.9 |
| Microsvasular Dermal EC TNFalpha + | 4.8 |
| IL-1beta | |
| Bronchial epithelium TNFalpha + | 2.3 |
| IL1beta | |
| Small airway epithelium none | 4.8 |
| Small airway epithelium TNFalpha + | 4.2 |
| IL-1beta | |
| Coronery artery SMC rest | 3.0 |
| Coronery artery SMC TNFalpha + | 4.6 |
| IL-1beta | |
| Astrocytes rest | 0.0 |
| Astrocytes TNFalpha + IL-1beta | 0.8 |
| KU-812 (Basophil) rest | 0.8 |
| KU-812 (Basophil) PMA/ionomycin | 3.6 |
| CCD1106 (Keratinocytes) none | 12.3 |
| CCD1106 (Keratinocytes) TNFalpha + | 11.3 |
| IL-1beta | |
| Liver cirrhosis | 6.2 |
| NCI-H292 none | 1.4 |
| NCI-H292 IL-4 | 5.8 |
| NCI-H292 IL-9 | 4.8 |
| NCI-H292 IL-13 | 2.1 |
| NCI-H292 IFN gamma | 0.9 |
| HPAEC none | 8.9 |
| HPAEC TNF alpha + IL-1 beta | 20.9 |
| Lung fibroblast none | 14.6 |
| Lung fibroblast TNF alpha + | 10.2 |
| IL-1 beta | |
| Lung fibroblast IL-4 | 1.5 |
| Lung fibroblast IL-9 | 3.4 |
| Lung fibroblast IL-13 | 2.3 |
| Lung fibroblast IFN gamma | 6.0 |
| Dermal fibroblast CCD1070 rest | 18.7 |
| Dermal fibroblast CCD1070 TNF alpha | 100.0 |
| Dermal fibroblast CCD1070 IL-1 beta | 8.4 |
| Dermal fibroblast IFN gamma | 19.3 |
| Dermal fibroblast IL-4 | 43.5 |
| Dermal Fibroblasts rest | 22.7 |
| Neutrophils TNFa + LPS | 0.8 |
| Neutrophils rest | 1.3 |
| Colon | 5.1 |
| Lung | 2.6 |
| Thymus | 12.1 |
| Kidney | 11.0 |

CNS_neurodegeneration_v1.0 Summary: Ag5721 This panel confirms the expression of this gene at moderate levels in the brain in an independent group of individuals. This gene is found to be upregulated in the temporal cortex of Alzheimer's disease patients. This gene encodes a putative kinesin, a microtubule-based motor protein involved in the transport of organelles. Axonal transport of APP in neurons is mediated by binding with kinesin. (Gunewardena S, Neuron Nov. 8, 2001;32(3):389-401). Kamal et al. suggest that impaired APP transport leads to enhanced axonal generation and deposition of Abeta, resulting in disruption of neurotrophic signaling and neurodegeneration (Nature Dec. 6, 2001;414(6864):643-8). Thus, therapeutic modulation of the expression or function of this gene may be useful in the treatment of neurodegenerative disorders, and specifically may decrease neuronal death and be of use in the treatment of Alzheimer's disease.

General_screening_panel_v1.5 Summary: Ag5721 Highest expression of this gene is seen in the fetal lung (CT=27.5). In addition, this gene is expressed at much higher levels in fetal lung tissue when compared to expression in the adult counterpart (CT=3 1). Thus, expression of this gene may be used to differentiate between the fetal and adult source of this tissue. In addition, therapeutic modulation of the expression or function of this gene may be useful in the treatment of diseases that affect the lung, including lung cancer.

Moderate to low levels of expression are seen in all regions of the CNS examined. Please see CNS_neurodegeneration_v1.0 for discussion of utility of this gene in CNS disorders.

Moderate to low levels of expression are also seen in pancreas, thyroid, fetal skeletal muscle, adipose and adult and fetal liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

Low but significant levels of expression are seen in many of the cancer cell lines on this panel. Interestingly, expression appears to be overexpressed in the normal tissue samples when compared to expression in the cell lines. Thus, modulation of the expression or function of this gene may be useful in the treatment of cancer.

Panel 4.1D Summary: Ag5721 Highest expression of this gene is seen in TNF-alpha treated dermal fibroblasts (CT=30.2). Moderate levels of expresison are also seen in resting NK cells. Low but significant levels of expression are seen in activated T cells, endothelial cells and lung and dermal fibroblasts. Thus, expression of this gene could be used as a marker of activated dermal fibroblasts and modulation of the gene product may be useful in the treatment of psoriasis.

AD. CG157629-01: Serine/Threonine Protein Phosphatase with EF-Hands-1.

Expression of gene CG157629-01 was assessed using the primer-probe set Ag5447, described in Table ADA. Please note that CG157629-01 represents a full-length physical clone.

TABLE ADA
Probe Name Ag5447

| | | | Start | SEQ ID |
|---|---|---|---|---|
| Primers | Sequence | Length | Position | No |
| Forward | 5′-ctggctcccaacgga-3′ | 15 | 906 | 558 |
| Probe | TET-5′-tggatctcctactgaacacttaacagagcatg-3′-TAMRA | 32 | 1002 | 559 |
| Reverse | 5′-acagaatatcaataatctgttcccat-3′ | 26 | 1035 | 560 |

AI_comprehensive panel_v1.0 Summary: Ag5447 Expression of this gene is low/undetectable in all samples on this panel (CTs>35). (Data not shown.)

General_screening_panel_v1.5 Summary: Ag5447 Expression of this gene is low/undetectable in all samples on this panel (CTs>35). (Data not shown.)

Panel 4.1D Summary: Ag5447 Expression of this gene is low/undetectable in all samples on this panel (CTs>35). (Data not shown.)

AE. CG157704-01: Kinesin 24.

Expression of gene CG157704-01 was assessed using the primer-probe set Ag5734, described in Table AEA. Results of the RTQ-PCR runs are shown in Tables AEB, AEC and AED.

TABLE AEA
Probe Name Ag5734

| | | | Start | SEQ ID |
|---|---|---|---|---|
| Primers | Sequence | Length | Position | No |
| Forward | 5′-gaggtacgtcgtggagaaatta-3′ | 22 | 718 | 561 |
| Probe | TET-5′-tcatgcacaagtagagtttctttgtcttc-3′-TAMRA | 29 | 754 | 562 |
| Reverse | 5′-tgaggtcaactgcttctttctt-3′ | 22 | 784 | 563 |

TABLE AEB
CNS_neurodegeneration_v1.0

| | Rel. Exp. (%) |
|---|---|
| | Ag5734, Run |
| Tissue Name | 247018774 |
| AD 1 Hippo | 15.3 |
| AD 2 Hippo | 15.9 |
| AD 3 Hippo | 9.0 |
| AD 4 Hippo | 8.7 |
| AD 5 Hippo | 68.8 |
| AD 6 Hippo | 57.4 |
| Control 2 Hippo | 29.1 |
| Control 4 Hippo | 24.3 |
| Control (Path) 3 Hippo | 20.4 |
| AD 1 Temporal Ctx | 17.8 |
| AD 2 Temporal Ctx | 36.9 |
| AD 3 Temporal Ctx | 13.9 |
| AD 4 Temporal Ctx | 24.5 |
| AD 5 Inf Temporal Ctx | 74.7 |
| AD 5 Sup Temporal Ctx | 41.8 |
| AD 6 Inf Temporal Ctx | 42.3 |
| AD 6 Sup Temporal Ctx | 66.4 |
| Control 1 Temporal Ctx | 20.2 |
| Control 2 Temporal Ctx | 33.4 |
| Control 3 Temporal Ctx | 15.7 |
| Control 3 Temporal Ctx | 3.0 |
| Control (Path) 1 Temporal Ctx | 50.0 |
| Control (Path) 2 Temporal Ctx | 39.0 |
| Control (Path) 3 Temporal Ctx | 2.6 |
| Control (Path) 4 Temporal Ctx | 64.6 |
| AD 1 Occipital Ctx | 20.2 |
| AD 2 Occipital Ctx (Missing) | 0.0 |
| AD 3 Occipital Ctx | 7.7 |
| AD 4 Occipital Ctx | 24.5 |
| AD 5 Occipital Ctx | 33.0 |
| AD 6 Occipital Ctx | 18.4 |
| Control 1 Occipital Ctx | 16.4 |
| Control 2 Occipital Ctx | 43.8 |
| Control 3 Occipital Ctx | 20.6 |
| Control 4 Occipital Ctx | 25.2 |
| Control (Path) 1 Occipital Ctx | 100.0 |
| Control (Path) 2 Occipital Ctx | 16.4 |
| Control (Path) 3 Occipital Ctx | 0.0 |
| Control (Path) 4 Occipital Ctx | 22.1 |
| Control 1 Parietal Ctx | 18.3 |
| Control 2 Parietal Ctx | 23.3 |
| Control 3 Parietal Ctx | 11.7 |
| Control (Path) 1 Parietal Ctx | 43.5 |
| Control (Path) 2 Parietal Ctx | 20.3 |
| Control (Path) 3 Parietal Ctx | 14.0 |
| Control (Path) 4 Parietal Ctx | 29.1 |

TABLE AEC
General_screening_panel_v1.5

| | Rel. Exp. (%) |
|---|---|
| | Ag5734, Run |
| Tissue Name | 245385008 |
| Adipose | 0.3 |
| Melanoma* Hs688(A).T | 2.7 |
| Melanoma* Hs688(B).T | 1.4 |
| Melanoma* M14 | 29.7 |
| Melanoma* LOXIMVI | 36.3 |
| Melanoma* SK-MEL-5 | 19.3 |
| Squamous cell carcinoma SCC-4 | 13.2 |
| Testis Pool | 3.3 |
| Prostate ca.* (bone met) PC-3 | 7.5 |
| Prostate Pool | 1.1 |
| Placenta | 3.8 |
| Uterus Pool | 1.3 |
| Ovarian ca. OVCAR-3 | 40.1 |
| Ovarian ca. SK-OV-3 | 1.3 |
| Ovarian ca. OVCAR-4 | 9.4 |
| Ovarian ca. OVCAR-5 | 31.2 |
| Ovarian ca. IGROV-1 | 10.9 |
| Ovarian ca. OVCAR-8 | 9.0 |
| Ovary | 3.8 |
| Breast ca. MCF-7 | 13.7 |
| Breast ca. MDA-MB-231 | 77.9 |
| Breast ca. BT 549 | 89.5 |
| Breast ca. T47D | 15.8 |
| Breast ca. MDA-N | 17.8 |
| Breast Pool | 2.9 |
| Trachea | 10.1 |
| Lung | 1.1. |
| Fetal Lung | 23.2 |
| Lung ca. NCI-N417 | 4.9 |
| Lung ca. LX-1 | 46.7 |
| Lung ca. NCI-H146 | 27.0 |
| Lung ca. SHP-77 | 31.4 |
| Lung ca. A549 | 44.1 |
| Lung ca. NCI-H526 | 10.0 |
| Lung ca. NCI-H23 | 1.7 |
| Lung ca. NCI-H460 | 0.1 |
| Lung ca. HOP-62 | 3.5 |
| Lung ca. NCI-H522 | 17.3 |
| Liver | 0.1 |
| Fetal Liver | 28.5 |
| Liver ca. HepG2 | 1.3 |
| Kidney Pool | 6.0 |
| Fetal Kidney | 19.2 |
| Renal ca. 786-0 | 23.3 |
| Renal ca. A498 | 9.3 |
| Renal ca. ACHN | 7.5 |
| Renal ca. UO-31 | 10.2 |
| Renal ca. TK-10 | 22.2 |
| Bladder | 10.2 |
| Gastric ca. (liver met.) NCI-N87 | 50.0 |
| Gastric ca. KATO III | 100.0 |
| Colon ca. SW-948 | 6.1 |
| Colon ca. SW480 | 68.3 |
| Colon ca.* (SW480 met) SW620 | 44.4 |
| Colon ca. HT29 | 23.8 |
| Colon ca. HCT-116 | 42.0 |
| Colon ca. CaCo-2 | 19.5 |
| Colon cancer tissue | 10.0 |
| Colon ca. SW1116 | 7.4 |
| Colon ca. Colo-205 | 9.4 |
| Colon ca. SW-48 | 11.7 |
| Colon Pool | 0.0 |
| Small Intestine Pool | 5.0 |
| Stomach Pool | 1.9 |
| Bone Marrow Pool | 1.3 |
| Fetal Heart | 6.8 |
| Heart Pool | 2.0 |
| Lymph Node Pool | 3.3 |
| Fetal Skeletal Muscle | 0.0 |
| Skeletal Muscle Pool | 2.1 |
| Spleen Pool | 1.4 |
| Thymus Pool | 16.3 |
| CNS cancer (glio/astro) U87-MG | 47.6 |
| CNS cancer (glio/astro) U-118-MG | 81.2 |
| CNS cancer (neuro; met) SK-N-AS | 26.4 |
| CNS cancer (astro) SF-539 | 26.1 |
| CNS cancer (astro) SNB-75 | 75.8 |
| CNS cancer (glio) SNB-19 | 8.4 |
| CNS cancer (glio) SF-295 | 20.9 |
| Brain (Amygdala) Pool | 1.4 |
| Brain (cerebellum) | 5.7 |
| Brain (fetal) | 11.0 |
| Brain (Hippocampus) Pool | 2.8 |
| Cerebral Cortex Pool | 4.8 |
| Brain (Substantia nigra) Pool | 2.9 |
| Brain (Thalamus) Pool | 4.6 |
| Brain (whole) | 4.8 |
| Spinal Cord Pool | 4.0 |
| Adrenal Gland | 3.2 |
| Pituitary gland Pool | 2.4 |
| Salivary Gland | 1.1 |
| Thyroid (female) | 3.5 |
| Pancreatic ca. CAPAN2 | 23.0 |
| Pancreas Pool | 1.9 |

TABLE AED
Panel 4.1D

| | Rel. Exp. (%) |
|---|---|
| | Ag5734, Run |
| Tissue Name | 246509244 |
| Secondary Th1 act | 65.5 |
| Secondary Th2 act | 98.6 |
| Secondary Tr1 act | 20.9 |
| Secondary Th1 rest | 0.0 |
| Secondary Th2 rest | 0.0 |
| Secondary Tr1 rest | 0.0 |
| Primary Th1 act | 0.4 |
| Primary Th2 act | 13.8 |
| Primary Tr1 act | 9.5 |
| Primary Th1 rest | 0.0 |
| Primary Th2 rest | 0.0 |
| Primary Tr1 rest | 0.0 |
| CD45RA CD4 lymphocyte act | 30.4 |
| CD45RO CD4 lymphocyte act | 43.2 |
| CD8 lymphocyte act | 3.9 |
| Secondary CD8 lymphocyte rest | 17.0 |
| Secondary CD8 lymphocyte act | 3.3 |
| CD4 lymphocyte none | 0.0 |
| 2ry Th1/Th2/Tr1_anti-CD95 CH11 | 1.7 |
| LAK cells rest | 8.1 |
| LAK cells IL-2 | 7.1 |
| LAK cells IL-2 + IL-12 | 2.7 |
| LAK cells IL-2 + IFN gamma | 4.0 |
| LAK cells IL-2 + IL-18 | 2.3 |
| LAK cells PMA/ionomycin | 15.8 |
| NK Cells IL-2 rest | 77.4 |
| Two Way MLR 3 day | 4.5 |
| Two Way MLR 5 day | 1.6 |
| Two Way MLR 7 day | 6.9 |
| PBMC rest | 0.0 |
| PBMC PWM | 3.8 |
| PBMC PHA-L | 8.8 |
| Ramos (B cell) none | 4.9 |
| Ramos (B cell) ionomycin | 35.4 |
| B lymphocytes PWM | 24.0 |
| B lymphocytes CD40L and IL-4 | 45.7 |
| EOL-1 dbcAMP | 60.7 |
| EOL-1 dbcAMP PMA/ionomycin | 3.2 |
| Dendritic cells none | 6.3 |
| Dendritic cells LPS | 0.7 |
| Dendritic cells anti-CD40 | 1.6 |
| Monocytes rest | 1.6 |
| Monocytes LPS | 3.7 |
| Macrophages rest | 3.8 |
| Macrophages LPS | 0.8 |
| HUVEC none | 10.1 |
| HUVEC starved | 36.9 |
| HUVEC IL-1 beta | 19.5 |
| HUVEC IFN gamma | 21.5 |
| HUVEC TNF alpha + IFN gamma | 2.1 |
| HUVEC TNF alpha + IL4 | 1.8 |
| HUVEC IL-11 | 9.0 |
| Lung Microvascular EC none | 12.2 |
| Lung Microvascular EC TNFalpha + IL-1beta | 2.7 |
| Microvascular Dermal EC none | 0.4 |
| Microsvasular Dermal EC TNFalpha + IL-1beta | 4.0 |
| Bronchial epithelium TNFalpha + IL1beta | 3.7 |
| Small airway epithelium none | 1.3 |
| Small airway epithelium TNFalpha + IL-1beta | 4.5 |
| Coronery artery SMC rest | 3.5 |
| Coronery artery SMC TNFalpha + IL-1beta | 2.9 |
| Astrocytes rest | 3.4 |
| Astrocytes TNFalpha + IL-1beta | 0.9 |
| KU-812 (Basophil) rest | 29.9 |
| KU-812 (Basophil) PMA/ionomycin | 40.9 |
| CCD1106 (Keratinocytes) none | 47.0 |
| CCD1106 (Keratinocytes) TNFalpha + IL-1beta | 20.7 |
| Liver cirrhosis | 2.0 |
| NCI-H292 none | 26.6 |
| NCI-H292 IL-4 | 30.6 |
| NCI-H292 IL-9 | 63.7 |
| NCI-H292 IL-13 | 29.3 |
| NCI-H292 IFN gamma | 16.0 |
| HPAEC none | 3.5 |
| HPAEC TNF alpha + IL-1 beta | 12.1 |
| Lung fibroblast none | 3.9 |
| Lung fibroblast TNF alpha + IL-1 beta | 5.4 |
| Lung fibroblast IL-4 | 1.0 |
| Lung fibroblast IL-9 | 6.2 |
| Lung fibroblast IL-13 | 0.0 |
| Lung fibroblast IFN gamma | 5.4 |
| Dermal fibroblast CCD1070 rest | 46.7 |
| Dermal fibroblast CCD1070 TNF alpha | 100.0 |
| Dermal fibroblast CCD1070 IL-1 beta | 22.5 |
| Dermal fibroblast IFN gamma | 16.6 |
| Dermal fibroblast IL-4 | 19.9 |
| Dermal Fibroblasts rest | 3.7 |
| Neutrophils TNFa + LPS | 1.6 |
| Neutrophils rest | 2.6 |
| Colon | 0.7 |
| Lung | 0.6 |
| Thymus | 12.3 |
| Kidney | 6.8 |

CNS_neurodegeneration_v1.0 Summary: Ag5734 This panel does not show differential expression of this gene in Alzheimer's disease. However, this profile confirms the expression of this gene at moderate levels in the brain. Please see Panel 1.5 for discussion of utility of this gene in the central nervous system.

General_screening_panel_v1.5 Summary: Ag5734 Highest expression of this gene is seen in a gastric cancer cell line (CT=29). This gene is widely expressed in this panel, with moderate expression seen in brain, colon, gastric, lung, breast, pancreatic, renal, ovarian, and melanoma cancer cell lines. This expression profile with prominent cell line expression suggests a role for this gene product in cell survival and proliferation. Modulation of this gene product may be useful in the treatment of cancer.

Among tissues with metabolic function, this gene is expressed at low but significant levels in pituitary, skeletal muscle, adrenal gland, pancreas, thyroid, fetal liver, and adult and fetal liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

This gene is also expressed at low but significant levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary: Ag5734 Highest expression is seen in TNF-a treated dermal fibroblasts. Low but significant expression is seen in activated T cells, resting NK cells, eosinophils, activated B cells, HUVECs, basophils and NCI-H292 goblet cells. This expression suggests that this gene product may be involved in autoinflammatory processes. Thus, expression of this gene could be used as a marker of activated dermal fibroblasts. Modulation of the expression or function of this gene may be useful in the treatment of RA, OA, lupus, asthma, allergy, emphysema, and psoriasis.

AF. CG158218-01: Kinesin 6.

Expression of gene CG158218-01 was assessed using the primer-probe set Ag5797, described in Table AFA. Results of the RTQ-PCR runs are shown in Tables AFB and AFC.

TABLE AFA
Probe Name Ag5797

| | | | Start | SEQ ID |
|---|---|---|---|---|
| Primers | Sequences | Length | Position | No |
| Forward | 5′-agttacaaaaggacagcagcaa-3′ | 22 | 621 | 564 |
| Probe | TET-5′-ccacattcattgtagatttccaaatagga-3′-TAMRA | 29 | 662 | 565 |
| Reverse | 5′-ttcatgtcttggatccaaaaga-3′ | 22 | 697 | 566 |

TABLE AFB
CNS_neurodegeneration_v1.0

| | Rel. Exp. (%) |
|---|---|
| | Ag5797, Run |
| Tissue Name | 247179625 |
| AD 1 Hippo | 15.9 |
| AD 2 Hippo | 32.1 |
| AD 3 Hippo | 6.8 |
| AD 4 Hippo | 9.5 |
| AD 5 Hippo | 27.4 |
| AD 6 Hippo | 33.9 |
| Control 2 Hippo | 31.0 |
| Control 4 Hippo | 25.2 |
| Control (Path) 3 Hippo | 7.9 |
| AD 1 Temporal Ctx | 80.7 |
| AD 2 Temporal Ctx | 33.2 |
| AD 3 Temporal Ctx | 9.3 |
| AD 4 Temporal Ctx | 24.0 |
| AD 5 Inf Temporal Ctx | 100.0 |
| AD 5 Sup Temporal Ctx | 51.1 |
| AD 6 Inf Temporal Ctx | 35.4 |
| AD 6 Sup Temporal Ctx | 29.1 |
| Control 1 Temporal Ctx | 7.0 |
| Control 2 Temporal Ctx | 22.5 |
| Control 3 Temporal Ctx | 20.6 |
| Control 3 Temporal Ctx | 5.6 |
| Control (Path) 1 Temporal Ctx | 48.0 |
| Control (Path) 2 Temporal Ctx | 29.5 |
| Control (Path) 3 Temporal Ctx | 4.8 |
| Control (Path) 4 Temporal Ctx | 22.5 |
| AD 1 Occipital Ctx | 12.8 |
| AD 2 Occipital Ctx (Missing) | 0.0 |
| AD 3 Occipital Ctx | 7.3 |
| AD 4 Occipital Ctx | 16.8 |
| AD 5 Occipital Ctx | 26.2 |
| AD 6 Occipital Ctx | 10.7 |
| Control 1 Occipital Ctx | 3.1 |
| Control 2 Occipital Ctx | 29.5 |
| Control 3 Occipital Ctx | 15.9 |
| Control 4 Occipital Ctx | 13.6 |
| Control (Path) 1 Occipital Ctx | 85.9 |
| Control (Path) 2 Occipital Ctx | 11.0 |
| Control (Path) 3 Occipital Ctx | 3.5 |
| Control (Path) 4 Occipital Ctx | 12.7 |
| Control 1 Parietal Ctx | 15.3 |
| Control 2 Parietal Ctx | 51.4 |
| Control 3 Parietal Ctx | 8.2 |
| Control (Path) 1 Parietal Ctx | 65.1 |
| Control (Path) 2 Parietal Ctx | 25.3 |
| Control (Path) 3 Parietal Ctx | 2.4 |
| Control (Path) 4 Parietal Ctx | 30.4 |

TABLE AFC
(general_screening_panel v1.5

| | Rel. Exp. (%) |
|---|---|
| | Ag5797, Run |
| Tissue Name | 245382863 |
| Adipose | 0.3 |
| Melanoma* Hs688(A).T | 0.1 |
| Melanoma* Hs688(B).T | 0.0 |
| Melanoma* M14 | 0.7 |
| Melanoma* LOXIMVI | 0.0 |
| Melanoma* SK-MEL-5 | 0.6 |
| Squamous cell carcinoma SCC-4 | 0.0 |
| Testis Pool | 9.9 |
| Prostate ca.* (bone met) PC-3 | 0.0 |
| Prostate Pool | 0.6 |
| Placenta | 0.1 |
| Uterus Pool | 0.2 |
| Ovarian ca. OVCAR-3 | 1.5 |
| Ovarian ca. SK-OV-3 | 2.0 |
| Ovarian ca. OVCAR-4 | 0.0 |
| Ovarian ca. OVCAR-5 | 1.2 |
| Ovarian ca. IGROV-1 | 0.1 |
| Ovarian ca. OVCAR-8 | 0.0 |
| Ovary | 1.4 |
| Breast ca. MCF-7 | 0.3 |
| Breast ca. MDA-MB-231 | 0.0 |
| Breast ca. BT 549 | 0.0 |
| Breast ca. T47D | 0.5 |
| Breast ca. MDA-N | 0.2 |
| Breast Pool | 1.3 |
| Trachea | 4.2 |
| Lung | 0.1 |
| Fetal Lung | 11.7 |
| Lung ca. NCI-N417 | 1.4 |
| Lung ca. LX-1 | 7.4 |
| Lung ca. NCI-H146 | 0.0 |
| Lung ca. SHP-77 | 1.7 |
| Lung ca. A549 | 0.0 |
| Lung ca. NCI-H526 | 0.2 |
| Lung ca. NCI-H23 | 0.3 |
| Lung ca. NCI-H460 | 0.0 |
| Lung ca. HOP-62 | 0.2 |
| Lung ca. NCI-H522 | 0.1 |
| Liver | 0.0 |
| Fetal Liver | 100.0 |
| Liver ca. HepG2 | 0.0 |
| Kidney Pool | 0.7 |
| Fetal Kidney | 4.7 |
| Renal ca. 786-0 | 0.1 |
| Renal ca. A498 | 0.1 |
| Renal ca. ACHN | 0.1 |
| Renal ca. UO-31 | 0.4 |
| Renal ca. TK-10 | 0.1 |
| Bladder | 0.6 |
| Gastric ca. (liver met.) NCI-N87 | 0.0 |
| Gastric ca. KATO III | 0.0 |
| Colon ca. SW-948 | 0.0 |
| Colon ca. SW480 | 4.2 |
| Colon ca.* (SW480 met) SW620 | 10.8 |
| Colon ca. HT29 | 0.0 |
| Colon ca. HCT-116 | 0.0 |
| Colon ca. CaCo-2 | 0.2 |
| Colon cancer tissue | 0.0 |
| Colon ca. SW1116 | 0.0 |
| Colon ca. Colo-205 | 0.0 |
| Colon ca. SW-48 | 0.1 |
| Colon Pool | 0.4 |
| Small Intestine Pool | 1.2 |
| Stomach Pool | 0.6 |
| Bone Marrow Pool | 0.2 |
| Fetal Heart | 0.0 |
| Heart Pool | 0.3 |
| Lymph Node Pool | 1.0 |
| Fetal Skeletal Muscle | 0.2 |
| Skeletal Muscle Pool | 0.1 |
| Spleen Pool | 0.1 |
| Thymus Pool | 1.4 |
| CNS cancer (glio/astro) U87-MG | 2.3 |
| CNS cancer (glio/astro) U-118-MG | 0.0 |
| CNS cancer (neuro; met) SK-N-AS | 0.4 |
| CNS cancer (astro) SF-539 | 0.0 |
| CNS cancer (astro) SNB-75 | 0.7 |
| CNS cancer (glio) SNB-19 | 0.4 |
| CNS cancer (glio) SF-295 | 0.7 |
| Brain (Amygdala) Pool | 7.1 |
| Brain (cerebellum) | 2.7 |
| Brain (fetal) | 2.1 |
| Brain (Hippocampus) Pool | 3.7 |
| Cerebral Cortex Pool | 6.3 |
| Brain (Substantia nigra) Pool | 9.7 |
| Brain (Thalamus) Pool | 4.0 |
| Brain (whole) | 2.8 |
| Spinal Cord Pool | 11.4 |
| Adrenal Gland | 0.3 |
| Pituitary gland Pool | 1.7 |
| Salivary Gland | 0.0 |
| Thyroid (female) | 0.7 |
| Pancreatic ca. CAPAN2 | 0.3 |
| Pancreas Pool | 0.8 |

CNS₁₃ neurodegeneration₁₃ v1.0 Summary: Ag5797 This panel does not show differential expression of this gene in Alzheimer's disease. However, this profile confirms the expression of this gene at moderate levels in the brain. Please see Panel 1.5 for discussion of utility of this gene in the central nervous system.

General₁₃ screening_panel_v1.5 Summary: Ag5797 Highest expression of this gene is seen in the fetal liver. Interestingly, this gene is expressed at much higher levels in fetal (CT=29) when compared to adult liver tissue (CT=40). This observation suggests that expression of this gene can be used to distinguish fetal from adult liver. In addition, the relative overexpression of this gene in fetal liver suggests that the protein product may enhance liver growth or development in the fetus and thus may also act in a regenerative capacity in the adult. Therefore, therapeutic modulation of the protein encoded by this gene could be useful in treatment of liver related diseases.

This gene is also expressed at low levels in the CNS, including the hippocampus, thalamus, substantia nigra, amygdala, cerebellum and cerebral cortex. Therefore, therapeutic modulation of the expression or function of this gene may be useful in the treatment of neurological disorders, such as Alzheimer's disease, Parkinson's disease, schizophrenia, multiple sclerosis, stroke and epilepsy.

Panel 4.1D Summary: Ag5797 Expression of this gene is low/undetectable in all samples on this panel (CTs>35). (Data not shown.)

AG. CG158583-01 and CG158583-04: Synaptic Vesicle Amine Transporter.

Expression of gene CG158583-01 and CG158583-04 was assessed using the primer-probe set Ag7590, described in Table AGA. Results of the RTQ-PCR runs are shown in Table AGB. Please note that CG158583-04 represents a full-length physical clone.

TABLE AGA
Probe Name Ag7590

| | | | Start | SEQ ID |
|---|---|---|---|---|
| Primers | Sequences | Length | Position | No |
| Forward | 5′-aactcctgacctcaggtgatc-3′ | 21 | 167 | 567 |
| Probe | TET-5′-tcctggaattacagtccccatcatcc-3′-TAMRA | 26 | 210 | 568 |
| Reverse | 5′-ctcatgcttaatgctgtacagataact-3′ | 27 | 238 | 569 |

TABLE AGB
Panel 5 Islet

| | Rel. Exp. (%) |
|---|---|
| | Ag7590, Run |
| Tissue Name | 310258790 |
| 97457_Patient-02go_adipose | 0.0 |
| 97476_Patient-07sk_skeletal muscle | 0.0 |
| 97477_Patient-07ut_uterus | 0.0 |
| 97478_Patient-07pl_placenta | 0.0 |
| 99167_Bayer Patient 1 | 100.0 |
| 97482_Patient-08ut_uterus | 12.2 |
| 97483_Patient-08pl_placenta | 0.0 |
| 97486_Patient-09sk_skeletal muscle | 10.2 |
| 97487_Patient-09ut_uterus | 0.0 |
| 97488_Patient-09pl_placenta | 0.0 |
| 97492_Patient-10ut_uterus | 21.6 |
| 97493_Patient-10pl_placenta | 0.0 |
| 97495_Patient-11go_adipose | 0.0 |
| 97496_Patient-11sk_skeletal muscle | 27.2 |
| 97497_Patient-11ut_uterus | 0.0 |
| 97498_Patient-11pl_placenta | 0.0 |
| 97500_Patient-12go_adipose | 32.3 |
| 97501_Patient-12sk_skeletal muscle | 0.0 |
| 97502_Patient-12ut_uterus | 13.3 |
| 97503_Patient-12pl_placenta | 0.0 |
| 94721_Donor2U-A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 0.0 |
| 73556_Heart_Cardiac stromal cells (primary) | 0.0 |
| 81735_Small Intestine | 26.6 |
| 72409_Kidney_Proximal Convoluted Tubule | 0.0 |
| 82685_Small intestine_Duodenum | 14.8 |
| 90650_Adrenal Adrenocortical adenoma | 0.0 |
| 72410_Kidney_HRCE | 0.0 |
| 72411_Kidney_HRE | 0.0 |
| 73139_Uterus_Uterine smooth muscle cells | 0.0 |

Panel 5 Islet Summary: Ag7590 Expression of this gene is restricted to a sample of pancreatic islet cells (CT=34.5). Thus, expression of this gene could be used to differentiate between this sample and other samples on this panel and as a marker of islet cells. Furthermore, therapeutic modulation of the expression or function of this gene may be useful in the treatment of diabetes.

AH. CG159084-01: Glutamate Decarboxylase like.

Expression of gene CG159084-01 was assessed using the primer-probe sets Ag5799 and Ag5799, described in Tables AHA and AHB.

TABLE AHA
Probe Name Ag5799

| | | | Start | SEQ ID |
|---|---|---|---|---|
| Primers | Sequences | Length | Position | No |
| Forward | 5′-agagatcaagaactccgaaagg-3′ | 22 | 1399 | 570 |
| Probe | TET-5′-tgccttccatcatcatctgtgcttta-3′-TAMRA | 26 | 1434 | 571 |
| Reverse | 5′-ggctggtagcttatcatgattg-3′ | 22 | 1460 | 572 |

TABLE AHB
Probe Name Ag5799

| | | | Start | SEQ ID |
|---|---|---|---|---|
| Primers | Sequences | Length | Position | No |
| Forward | 5′-agagatcaagaactccgaaagg-3′ | 22 | 1399 | 573 |
| Probe | TET-5′-tgccttccatcatcatctgtgcttta-3′-TAMRA | 26 | 1434 | 574 |
| Reverse | 5′-ggctggtagcttatcatgattg-3′ | 22 | 1460 | 575 |

CNS_neurodegeneration_v1.0 Summary: Ag5799 Expression of this gene is low/undetectable in all samples on this panel (CTs>35). (Data not shown.)

General_screening_panelᵥ1.5 Summary: Ag5799 Expression of this gene is low/undetectable in all samples on this panel (CTs>35). (Data not shown.)

General_screening_panelᵥ1.6 Summary: Ag5799 Expression of this gene is low/undetectable in all samples on this panel (CTs>35). (Data not shown.)

Panel 4.1D Summary: Ag5799 Expression of this gene is low/undetectable in all samples on this panel (CTs>35). (Data not shown.)

Panel 5 Islet Summary: Ag5799 Expression of this gene is low/undetectable in all samples on this panel (CTs>35). (Data not shown.)

Panel CNS_{—}1.1 Summary: Ag5799 Expression of this gene is low/undetectable in all samples on this panel (CTs>35). (Data not shown.)

AI. CG159130-01: Hyperpolarization-Activated Cyclic Nucleotide-Gated Channel 1.

Expression of gene CG159130-01 was assessed using the primer-probe set Ag7494, described in Table AIA. Results of the RTQ-PCR runs are shown in Table AIB.

TABLE AIA
Probe Name Ag7494

| | | | Start | SEQ ID |
|---|---|---|---|---|
| Primers | Sequences | Length | Position | No |
| Forward | 5′-ttcatacgcactcttcaaagcta-3′ | 23 | 1095 | 576 |
| Probe | TET-5′-cccagtcagcatgtctgacctctgga-3′-TAMRA | 26 | 1155 | 577 |
| Reverse | 5′-cgacgatcatgctcagcat-3′ | 19 | 1186 | 578 |

TABLE AIB
CNS_neurodegeneration_v1.0

| | Rel. Exp. (%) |
|---|---|
| | Ag7494, Run |
| Tissue Name | 308752180 |
| AD 1 Hippo | 2.1 |
| AD 2 Hippo | 7.9 |
| AD 3 Hippo | 2.2 |
| AD 4 Hippo | 2.0 |
| AD 5 hippo | 100.0 |
| AD 6 Hippo | 17.6 |
| Control 2 Hippo | 21.6 |
| Control 4 Hippo | 1.1 |
| Control (Path) 3 Hippo | 0.6 |
| AD 1 Temporal Ctx | 3.0 |
| AD 2 Temporal Ctx | 9.1 |
| AD 3 Temporal Ctx | 1.0 |
| AD 4 Temporal Ctx | 5.1 |
| AD 5 Inf Temporal Ctx | 69.3 |
| AD 5 Sup Temporal Ctx | 15.0 |
| AD 6 Inf Temporal Ctx | 14.6 |
| AD 6 Sup Temporal Ctx | 19.8 |
| Control 1 Temporal Ctx | 0.6 |
| Control 2 Temporal Ctx | 34.9 |
| Control 3 Temporal Ctx | 6.2 |
| Control 4 Temporal Ctx | 1.8 |
| Control (Path) 1 Temporal Ctx | 43.5 |
| Control (Path) 2 Temporal Ctx | 19.6 |
| Control (Path) 3 Temporal Ctx | 0.8 |
| Control (Path) 4 Temporal Ctx | 13.7 |
| AD 1 Occipital Ctx | 6.8 |
| AD 2 Occipital Ctx (Missing) | 0.0 |
| AD 3 Occipital Ctx | 1.6 |
| AD 4 Occipital Ctx | 8.8 |
| AD 5 Occipital Ctx | 12.2 |
| AD 6 Occipital Ctx | 57.4 |
| Control 1 Occipital Ctx | 0.5 |
| Control 2 Occipital Ctx | 70.2 |
| Control 3 Occipital Ctx | 7.4 |
| Control 4 Occipital Ctx | 1.1 |
| Control (Path) 1 Occipital Ctx | 62.9 |
| Control (Path) 2 Occipital Ctx | 3.8 |
| Control (Path) 3 Occipital Ctx | 0.6 |
| Control (Path) 4 Occipital Ctx | 7.2 |
| Control 1 Parietal Ctx | 0.9 |
| Control 2 Parietal Ctx | 16.4 |
| Control 3 Parietal Ctx | 11.5 |
| Control (Path) 1 Parietal Ctx | 66.0 |
| Control (Path) 2 Parietal Ctx | 11.7 |
| Control (Path) 3 Parietal Ctx | 0.9 |
| Control (Path) 4 Parietal Ctx | 31.9 |

CNS_neurodegeneration_v1.0 Summary: Ag7494 This panel does not show differential expression of this gene in Alzheimer's disease. However, this profile confirms the expression of this gene at high to moderate levels in the brain. Therefore, therapeutic modulation of the expression or function of this gene may be useful in the treatment of neurological disorders, such as Alzheimer's disease, Parkinson's disease, schizophrenia, multiple sclerosis, stroke and epilepsy.

AJ. CG159178-01: Carbonic Anhydrase VI Precursor.

Expression of gene CG159178-01 was assessed using the primer-probe set Ag4880, described in Table AJA. Results of the RTQ-PCR runs are shown in Tables AJB, AJC and AJD.

TABLE AJA
Probe Name Ag4880

| | | | Start | SEQ ID |
|---|---|---|---|---|
| Primers | Sequences | Length | Position | No |
| Forward | 5′-ttcgttgaggtgaagaattacc-3′ | 22 | 319 | 579 |
| Probe | TET-5′-cagcaacttcatttctcatctggcca-3′-TAMRA | 26 | 357 | 580 |
| Reverse | 5′-gttctttgtcctgggtacttga-3′ | 22 | 386 | 581 |

TABLE AJB
General_screening_panel_v1.5

| | Rel. Exp. (%) |
|---|---|
| | Ag4880, Run |
| Tissue Name | 228806989 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 0.1 |
| Lung | 0.0 |
| Fetal Lung | 0.0 |
| Lung ca. NCI-N417 | 0.0 |
| Lung ca. LX-1 | 1.4 |
| Lung ca. NCI-H146 | 0.0 |
| Lung ca. SHP-77 | 0.3 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro;met) SK-N-AS | 0.0 |
| CNS cancer (astro) SF-539 | 0.0 |
| CNS cancer (astro)SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 0.0 |
| Adrenal Gland | 0.0 |
| Pituitary gland Pool | 0.0 |
| Salivary Gland | 100.0 |
| Thyroid (female) | 0.0 |
| Pancreatic ca. CAPAN2 | 0.0 |
| Pancreas Pool | 0.0 |

TABLE AJC
Panel 4.1D

| | Rel. Exp. (%) |
|---|---|
| | Ag4880, Run |
| Tissue Name | 223350178 |
| Secondary Th1 act | 100.0 |
| Secondary Th2 act | 0.0 |
| Secondary Tr1 act | 7.2 |
| Secondary Th1 rest | 11.3 |
| Secondary Th2 rest | 7.0 |
| Secondary Tr1 rest | 8.8 |
| Primary Th1 act | 5.4 |
| Primary Th2 act | 0.0 |
| Primary Tr1 act | 43.2 |
| Primary Th1 rest | 29.5 |
| Primary Th2 rest | 6.7 |
| Primary Tr1 rest | 10.4 |
| CD45RA CD4 lymphocyte act | 19.2 |
| CD45RO CD4 lymphocyte act | 22.5 |
| CD8 lymphocyte act | 31.6 |
| Secondary CD8 lymphocyte rest | 5.4 |
| Secondary CD8 lymphocyte act | 10.6 |
| CD4 lymphocyte none | 10.6 |
| 2ry Th1/Th2/Tr1_anti-CD95 CH11 | 10.5 |
| LAK cells rest | 4.7 |
| LAK cells IL-2 | 19.1 |
| LAK cells IL-2 + IL-12 | 56.3 |
| LAK cells IL-2 + IFN gamma | 28.3 |
| LAK cells IL-2 + IL-18 | 33.4 |
| LAK cells PMA/ionomycin | 0.0 |
| NK Cells IL-2 rest | 40.9 |
| Two Way MLR 3 day | 13.9 |
| Two Way MLR 5 day | 3.4 |
| Two Way MLR 7 day | 25.7 |
| PBMC rest | 4.9 |
| PBMC PWM | 21.3 |
| PBMC PHA-L | 17.6 |
| Ramos (B cell) none | 4.7 |
| Ramos (B cell) ionomycin | 10.6 |
| B lymphocytes PWM | 5.4 |
| B lymphocytes CD40L and IL-4 | 6.7 |
| EOL-1 dbcAMP | 31.4 |
| EOL-1 dbcAMP PMA/ionomycin | 3.5 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.7 |
| Macrophages rest | 0.0 |
| Macrophages LPS | 0.0 |
| HUVEC none | 11.3 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 0.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 3.9 |
| Dermal fibroblast CCD1070 TNF alpha | 53.2 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 0.0 |
| Lung | 5.3 |
| Thymus | 19.6 |
| Kidney | 3.2 |

TABLE AJD
Panel 5 Islet

| | Rel. Exp. (%) |
|---|---|
| | Ag4880, Run |
| Tissue Name | 296908323 |
| 97457_Patient-02go_adipose | 0.0 |
| 97476_Patient-07sk_skeletal muscle | 0.0 |
| 97477_Patient-07ut_uterus | 0.0 |
| 97478_Patient-07pl_placenta | 0.0 |
| 99167_Bayer Patient 1 | 0.0 |
| 97482_Patient-08ut_uterus | 0.0 |
| 97483_Patient-08pl_placenta | 0.0 |
| 97486_Patient-09sk_skeletal muscle | 0.0 |
| 97487_Patient-09ut_uterus | 0.0 |
| 97488_Patient-09pl_placenta | 0.0 |
| 97492_Patient-10ut_uterus | 0.0 |
| 97493_Patient-10pl_placenta | 0.0 |
| 97495_Patient-11go_adipose | 0.0 |
| 97496_Patient-11sk_skeletal muscle | 0.0 |
| 97497_Patient-11ut_uterus | 0.0 |
| 97498_Patient-11pl_placenta | 0.0 |
| 97500_Patient-12go_adipose | 0.0 |
| 97501_Patient-12sk_skeletal muscle | 0.0 |
| 97502_Patient-12ut_uterus | 0.0 |
| 97503_Patient-12pl_placenta | 0.0 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 0.0 |
| 73556_Heart_Cardiac stromal cells (primary) | 0.0 |
| 81735_Small Intestine | 100.0 |
| 72409_Kidney_Proximal Convoluted Tubule | 0.0 |
| 82685_Small intestine_Duodenum | 0.0 |
| 90650_Adrenal_Adrenocortical adenoma | 0.0 |
| 72410_Kidney_HRCE | 0.0 |
| 72411_Kidney_HRE | 0.0 |
| 73139_Uterus_Uterine smooth muscle cells | 0.0 |

General_screening_panel_v1.5 Summary: Ag4880 Expression of this gene is highest in salivary gland (CT=20.3). Thus expression of this gene could be used to differentiate between this sample and other samples on this panel and as a marker of this tissue.

Panel 4.1D Summary: Ag4880 Highest expression of this gene is seen a sample derived from chronically activated Th1 cells (CT=32.2). Low but significant expression is seen in primary activated Th1 and Th2 cells, LAK cells, NK cells, eosinophils, TNF-a activated dermal fibroblasts and thymus. This expression profile suggests that this gene product may be involved in autoimmune disease.

Panel 5 Islet Summary: Ag4880 Expression of this gene is limited to the small intestine (CT=23.7). Thus expression of this gene could be used to differentiate between this sample and other samples on this panel and as a marker of this tissue.

AK. CG160131-01: Glycerol Kinase.

Expression of gene CG160131-01 was assessed using the primer-probe set Ag5581, described in Table AKA. Results of the RTQ-PCR runs are shown in Tables AKB, AKC, AKD, AKE, AKF, AKG and AKH.

TABLE AKA
Probe Name Ag5581

| | | | Start | SEQ ID |
|---|---|---|---|---|
| Primers | Sequences | Length | Position | No |
| Forward | 5′-accactgtagtctgggacaaga-3′ | 22 | 292 | 582 |
| Probe | TET-5′-tctacaatgctgtggctgctccagtt-3′-TAMRA | 26 | 329 | 583 |
| Reverse | 5′-acggcaactggaactgaag-3′ | 19 | 365 | 584 |

TABLE AKB
AI_comprehensive panel_v1.0

| | Rel. Exp. (%) | Rel. Exp. (%) |
|---|---|---|
| | Ag5581, Run | Ag5581, Run |
| Tissue Name | 244333633 | 244899563 |
| 110967 COPD-F | 0.0 | 0.0 |
| 110980 COPD-F | 0.0 | 0.0 |
| 110968 COPD-M | 3.9 | 0.0 |
| 110977 COPD-M | 0.0 | 9.0 |
| 110989 | 0.0 | 7.4 |
| Emphysema-F | | |
| 110992 | 0.0 | 0.0 |
| Emphysema-F | | |
| 110993 | 4.2 | 0.0 |
| Emphysema-F | | |
| 110994 | 0.0 | 0.0 |
| Emphysema-F | | |
| 110995 | 14.0 | 3.6 |
| Emphysema-F | | |
| 110996 | 0.0 | 0.0 |
| Emphysema-F | | |
| 110997 | 3.9 | 13.3 |
| Asthma-M | | |
| 111001 | 0.0 | 0.0 |
| Asthma-F | | |
| 111002 | 0.0 | 6.1 |
| Asthma-F | | |
| 111003 Atopic | 4.3 | 0.0 |
| Asthma-F | | |
| 111004 Atopic | 0.0 | 0.0 |
| Asthma-F | | |
| 111005 Atopic | 0.0 | 8.0 |
| Asthma-F | | |
| 111006 Atopic | 0.0 | 0.0 |
| Asthma-F | | |
| 111417 | 0.0 | 0.0 |
| Allergy-M | | |
| 112347 | 0.0 | 0.0 |
| Allergy-M | | |
| 112349 Normal | 0.0 | 0.0 |
| Lung-F | | |
| 112357 Normal | 0.0 | 0.0 |
| Lung-F | | |
| 112354 Normal | 0.0 | 0.0 |
| Lung-M | | |
| 112374 | 14.9 | 16.2 |
| Crohns-F | | |
| 112389 Match | 0.0 | 0.0 |
| Control | | |
| Crohns-F | | |
| 112375 | 0.0 | 4.5 |
| Crohns-F | | |
| 112732 Match | 0.0 | 6.2 |
| Control | | |
| Crohns-F | | |
| 112725 | 0.0 | 0.0 |
| Crohns-M | | |
| 112387 Match | 0.0 | 7.6 |
| Control | | |
| Crohns-M | | |
| 112378 | 0.0 | 0.0 |
| Crohns-M | | |
| 112390 Match | 5.5 | 7.1 |
| Control | | |
| Crohns-M | | |
| 112726 | 1.8 | 3.8 |
| Crohns-M | | |
| 112731 Match | 1.3 | 7.7 |
| Control | | |
| Crohns-M | | |
| 112380 Ulcer | 3.9 | 8.3 |
| Col-F | | |
| 112734 Match | 100.0 | 100.0 |
| Control Ulcer | | |
| Col-F | | |
| 112384 Ulcer | 3.7 | 0.0 |
| Col-F | | |
| 112737 Match | 0.0 | 0.0 |
| Control Ulcer | | |
| Col-F | | |
| 112386 Ulcer | 4.2 | 0.0 |
| Col-F | | |
| 112738 Match | 15.5 | 66.0 |
| Control Ulcer | | |
| Col-F | | |
| 112381 Ulcer | 0.0 | 0.0 |
| Col-M | | |
| 112735 Match | 17.9 | 9.3 |
| Control Ulcer | | |
| Col-M | | |
| 112382 Ulcer | 3.2 | 0.0 |
| Col-M | | |
| 112394 Match | 0.0 | 0.0 |
| Control Ulcer | | |
| Col-M | | |
| 112383 Ulcer | 1.3 | 0.0 |
| Col-M | | |
| 112736 Match | 0.0 | 11.9 |
| Control Ulcer | | |
| Col-M | | |
| 112423 | 10.6 | 22.1 |
| Psoriasis-F | | |
| 112427 Match | 0.0 | 6.7 |
| Control | | |
| Psoriasis-F | | |
| 112418 | 0.0 | 0.0 |
| Psoriasis-M | | |
| 112723 Match | 0.0 | 0.0 |
| Control | | |
| Psoriasis-M | | |
| 112419 | 0.0 | 0.0 |
| Psoriasis-M | | |
| 112424 Match | 3.4 | 4.1 |
| Control | | |
| Psoriasis-M | | |
| 112420 | 12.0 | 8.2 |
| Psoriasis-M | | |
| 112425 Match | 0.0 | 0.0 |
| Control | | |
| Psoriasis-M | | |
| 104689 (MF) | 13.9 | 13.5 |
| OA Bone- | | |
| Backus | | |
| 104690 (MF) | 0.0 | 15.8 |
| Adj “Normal” | | |
| Bone-Backus | | |
| 104691 (MF) | 4.5 | 0.0 |
| OA | | |
| Synovium- | | |
| Backus | | |
| 104692 (BA) | 0.0 | 0.0 |
| OA Cartilage- | | |
| Backus | | |
| 104694 (BA) | 18.7 | 21.0 |
| OA Bone- | | |
| Backus | | |
| 104695 (BA) | 0.0 | 8.4 |
| Adj “Normal” | | |
| Bone-Backus | | |
| 104696 (BA) | 23.7 | 15.5 |
| OA | | |
| Synovium- | | |
| Backus | | |
| 104700 (SS) | 3.7 | 8.6 |
| OA Bone- | | |
| Backus | | |
| 104701 (SS) | 5.6 | 27.5 |
| Adj “Normal” | | |
| Bone-Backus | | |
| 104702 (SS) | 7.3 | 0.0 |
| OA | | |
| Synovium- | | |
| Backus | | |
| 117093 OA | 0.0 | 0.0 |
| Cartilage | | |
| Rep7 | | |
| 112672 OA | 7.6 | 3.8 |
| Bone5 | | |
| 112673 OA | 7.6 | 7.7 |
| Synovium5 | | |
| 112674 OA | 2.3 | 9.7 |
| Synovial Fluid | | |
| cells5 | | |
| 117100 OA | 0.0 | 0.0 |
| Cartilage | | |
| Rep14 | | |
| 112756 OA | 7.7 | 0.0 |
| Bone9 | | |
| 112757 OA | 10.6 | 9.7 |
| Synovium9 | | |
| 112758 OA | 0.0 | 0.0 |
| Synovial Fluid | | |
| Cells9 | | |
| 117125 RA | 0.0 | 0.0 |
| Cartilage | | |
| Rep2 | | |
| 113492 Bone2 | 66.0 | 40.9 |
| RA | | |
| 113493 | 7.5 | 7.5 |
| Synovium2 | | |
| RA | | |
| 113494 Syn | 23.3 | 46.0 |
| Fluid Cells | | |
| RA | | |
| 113499 | 13.6 | 33.4 |
| Cartilage4 RA | | |
| 113500 Bone4 | 68.8 | 37.1 |
| RA | | |
| 113501 | 29.9 | 54.3 |
| Synovium4 | | |
| RA | | |
| 113502 Syn | 3.8 | 28.3 |
| Fluid Cells4 | | |
| RA | | |
| 113495 | 37.9 | 68.3 |
| Cartilage3 RA | | |
| 113496 Bone3 | 23.3 | 30.4 |
| RA | | |
| 113497 | 27.7 | 0.0 |
| Synovium3 | | |
| RA | | |
| 113498 Syn | 52.9 | 82.9 |
| Fluid Cells3 | | |
| RA | | |
| 117106 | 0.0 | 0.0 |
| Normal | | |
| Cartilage | | |
| Rep20 | | |
| 113663 Bone3 | 8.1 | 0.0 |
| Normal | | |
| 113664 | 0.0 | 0.0 |
| Synovium3 | | |
| Normal | | |
| 113665 Syn | 0.0 | 0.0 |
| Fluid Cells3 | | |
| Normal | | |
| 117107 | 0.0 | 0.0 |
| Normal | | |
| Cartilage | | |
| Rep22 | | |
| 113667 Bone4 | 4.6 | 0.0 |
| Normal | | |
| 113668 | 0.0 | 8.9 |
| Synovium4 | | |
| Normal | | |
| 113669 Syn | 0.0 | 0.0 |
| Fluid Cells4 | | |
| Normal | | |

TABLE AKC
General_screening_panel_v1.5

| | Rel. Exp. (%) |
|---|---|
| | Ag5581, Run |
| Tissue Name | 244896891 |
| Adipose | 1.9 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 2.0 |
| Squamous cell carcinoma SCC-4 | 0.0 |
| Testis Pool | 0.0 |
| Prostate ca.* (bone met) PC-3 | 0.5 |
| Prostate Pool | 0.6 |
| Placenta | 0.7 |
| Uterus Pool | 0.0 |
| Ovarian ca. OVCAR-3 | 0.4 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 2.0 |
| Ovarian ca. OVCAR-8 | 3.4 |
| Ovary | 0.0 |
| Breast ca. MCF-7 | 0.5 |
| Breast ca. MDA-MB-231 | 0.7 |
| Breast ca. BT 549 | 0.2 |
| Breast ca. T47D | 0.0 |
| Breast ca. MDA-N | 2.4 |
| Breast Pool | 0.0 |
| Trachea | 3.3 |
| Lung | 0.0 |
| Fetal Lung | 5.9 |
| Lung ca. NCI-N417 | 0.0 |
| Lung ca. LX-1 | 0.0 |
| Lung ca. NCI-H146 | 1.2 |
| Lung ca. SHP-77 | 0.0 |
| Lung ca. A549 | 0.0 |
| Lung ca. NCI-H526 | 0.0 |
| Lung ca. NCI-H23 | 0.6 |
| Lung ca. NCI-H460 | 0.0 |
| Lung ca. HOP-62 | 0.0 |
| Lung ca. NCI-H522 | 0.0 |
| Liver | 8.1 |
| Fetal Liver | 100.0 |
| Liver ca. HepG2 | 42.6 |
| Kidney Pool | 1.1 |
| Fetal Kidney | 2.2 |
| Renal ca. 786-0 | 0.5 |
| Renal ca. A498 | 0.0 |
| Renal ca. ACHN | 1.6 |
| Renal ca. UO-31 | 0.0 |
| Renal ca. TK-10 | 22.2 |
| Bladder | 22.1 |
| Gastric ca. (liver met.) NCI-N87 | 0.0 |
| Gastric ca. KATO III | 1.6 |
| Colon ca. SW-948 | 1.3 |
| Colon ca. SW480 | 1.2 |
| Colon ca.* (SW480 met) SW620 | 0.0 |
| Colon ca. HT29 | 0.0 |
| Colon ca. HCT-116 | 0.0 |
| Colon ca. CaCo-2 | 6.0 |
| Colon cancer tissue | 27.2 |
| Colon ca. SW1116 | 0.0 |
| Colon ca. Colo-205 | 0.7 |
| Colon ca. SW-48 | 0.6 |
| Colon Pool | 0.0 |
| Small Intestine Pool | 1.5 |
| Stomach Pool | 0.5 |
| Bone Marrow Pool | 0.6 |
| Fetal Heart | 0.8 |
| Heart Pool | 0.0 |
| Lymph Node Pool | 0.6 |
| Fetal Skeletal Muscle | 0.0 |
| Skeletal Muscle Pool | 15.9 |
| Spleen Pool | 0.6 |
| Thymus Pool | 0.6 |
| CNS cancer (glio/astro) U87-MG | 2.6 |
| CNS cancer (glio/astro) U-118-MG | 4.0 |
| CNS cancer (neuro; met) SK-N-AS | 0.0 |
| CNS cancer (astro) SF-539 | 0.0 |
| CNS cancer (astro) SNB-75 | 2.4 |
| CNS cancer (glio) SNB-19 | 4.6 |
| CNS cancer (glio) SF-295 | 1.0 |
| Brain (Amygdala) Pool | 1.7 |
| Brain (cerebellum) | 3.7 |
| Brain (fetal) | 0.0 |
| Brain (Hippocampus) Pool | 7.6 |
| Cerebral Cortex Pool | 1.3 |
| Brain (Substantia nigra) Pool | 3.9 |
| Brain (Thalamus) Pool | 1.5 |
| Brain (whole) | 4.2 |
| Spinal Cord Pool | 15.1 |
| Adrenal Gland | 1.0 |
| Pituitary gland Pool | 0.0 |
| Salivary Gland | 0.7 |
| Thyroid (female) | 1.0 |
| Pancreatic ca. CAPAN2 | 0.0 |
| Pancreas Pool | 1.5 |

TABLE AKD
General_screening_panel_v1.6

| | Rel. Exp. (%) |
|---|---|
| | Ag5581, Run |
| Tissue Name | 278988931 |
| Adipose | 6.1 |
| Melanoma* Hs688(A).T | 0.0 |
| Melanoma* Hs688(B).T | 0.0 |
| Melanoma* M14 | 3.8 |
| Melanoma* LOXIMVI | 0.9 |
| Melanoma* SK-MEL-5 | 3.8 |
| Squamous cell carcinoma SCC-4 | 0.0 |
| Testis Pool | 0.0 |
| Prostate ca.* (bone met) PC-3 | 2.7 |
| Prostate Pool | 2.5 |
| Placenta | 0.0 |
| Uterus Pool | 0.0 |
| Ovarian ca. OVCAR-3 | 0.7 |
| Ovarian ca. SK-OV-3 | 0.7 |
| Ovarian ca. OVCAR-4 | 0.0 |
| Ovarian ca. OVCAR-5 | 0.0 |
| Ovarian ca. IGROV-1 | 1.6 |
| Ovarian ca. OVCAR-8 | 3.4 |
| Ovary | 0.9 |
| Breast ca. MCF-7 | 0.0 |
| Breast ca. MDA-MB-231 | 0.0 |
| Breast ca. BT 549 | 0.0 |
| Breast ca. T47D | 0.8 |
| Breast ca. MDA-N | 0.8 |
| Breast Pool | 0.6 |
| Trachea | 5.1 |
| Lung | 0.0 |
| Fetal Lung | 7.0 |
| Lung ca. NCI-N417 | 0.0 |
| Lung ca. LX-1 | 0.0 |
| Lung ca. NCI-H146 | 0.0 |
| Lung ca. SHP-77 | 0.9 |
| Lung ca. A549 | 1.0 |
| Lung ca. NCI-H526 | 0.0 |
| Lung ca. NCI-H23 | 0.0 |
| Lung ca. NCI-H460 | 0.8 |
| Lung ca. HOP-62 | 0.7 |
| Lung ca. NCI-H522 | 0.0 |
| Liver | 3.9 |
| Fetal Liver | 100.0 |
| Liver ca. HepG2 | 29.9 |
| Kidney Pool | 0.5 |
| Fetal Kidney | 5.9 |
| Renal ca. 786-0 | 0.0 |
| Renal ca. A498 | 0.0 |
| Renal ca. ACHN | 0.0 |
| Renal ca. UO-31 | 2.9 |
| Renal ca. TK-10 | 14.8 |
| Bladder | 27.9 |
| Gastric ca. (liver met.) NCI-N87 | 1.7 |
| Gastric ca. KATO III | 1.2 |
| Colon ca. SW-948 | 0.0 |
| Colon ca. SW480 | 1.2 |
| Colon ca.* (SW480 met) SW620 | 0.9 |
| Colon ca. HT29 | 0.0 |
| Colon ca. HCT-116 | 0.0 |
| Colon ca. CaCo-2 | 5.7 |
| Colon cancer tissue | 20.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 1.6 |
| Stomach Pool | 3.7 |
| Bone Marrow Pool | 0.0 |
| Fetal Heart | 2.7 |
| Heart Pool | 1.4 |
| Lymph Node Pool | 0.3 |
| Fetal Skeletal Muscle | 1.0 |
| Skeletal Muscle Pool | 2.8 |
| Spleen Pool | 3.8 |
| Thymus Pool | 1.6 |
| CNS cancer (glio/astro) U87-MG | 1.9 |
| CNS cancer (glio/astro) U-118-MG | 3.2 |
| CNS cancer (neuro; met) SK-N-AS | 0.0 |
| CNS cancer (astro) SF-539 | 0.0 |
| CNS cancer (astro) SNB-75 | 0.8 |
| CNS cancer (glio) SNB-19 | 2.6 |
| CNS cancer (glio) SF-295 | 2.8 |
| Brain (Amygdala) Pool | 5.2 |
| Brain (cerebellum) | 0.0 |
| Brain (fetal) | 1.9 |
| Brain (Hippocampus) Pool | 14.1 |
| Cerebral Cortex Pool | 6.5 |
| Brain (Substantia nigra) Pool | 6.6 |
| Brain (Thalamus) Pool | 12.0 |
| Brain (whole) | 5.4 |
| Spinal Cord Pool | 12.1 |
| Adrenal Gland | 4.2 |
| Pituitary gland Pool | 0.8 |
| Salivary Gland | 0.8 |
| Thyroid (female) | 0.9 |
| Pancreatic ca. CAPAN2 | 0.0 |
| Pancreas Pool | 0.0 |

TABLE AKE
Panel 4.1D

| | Rel. Exp. (%) |
|---|---|
| | Ag5581, Run |
| Tissue Name | 244337065 |
| Secondary Th1 act | 0.1 |
| Secondary Th2 act | 0.2 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.2 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.1 |
| CD45RO CD4 lymphocyte act | 0.3 |
| CD8 lymphocyte act | 0.0 |
| Secondary CD8 lymphocyte rest | 0.1 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 2.3 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.1 |
| LAK cells PMA/ionomycin | 21.2 |
| NK Cells IL-2 rest | 0.0 |
| Two Way MLR 3 day | 1.9 |
| Two Way MLR 5 day | 0.1 |
| Two Way MLR 7 day | 0.1 |
| PBMC rest | 0.0 |
| PBMC PWM | 0.1 |
| PBMC PHA-L | 0.5 |
| Ramos (B cell) none | 0.0 |
| Ramos (B cell) ionomycin | 0.0 |
| B lymphocytes PWM | 0.1 |
| B lymphocytes CD40L and IL-4 | 0.1 |
| EOL-1 dbcAMP | 0.0 |
| EOL-1 dbcAMP PMA/ionomycin | 0.0 |
| Dendritic cells none | 2.3 |
| Dendritic cells LPS | 1.5 |
| Dendritic cells anti-CD40 | 0.3 |
| Monocytes rest | 0.0 |
| Monocytes LPS | 100.0 |
| Macrophages rest | 1.0 |
| Macrophages LPS | 1.5 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.1 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.3 |
| CCD1106 (Keratinocytes) none | 0.0 |
| CCD1106 (Keratinocytes) TNFalpha + IL-1beta | 0.0 |
| Liver cirrhosis | 0.3 |
| NCI-H292 none | 0.0 |
| NCI-H292 IL-4 | 0.2 |
| NCI-H292 IL-9 | 0.1 |
| NCI-H292 IL-13 | 0.1 |
| NCI-H292 IFN gamma | 0.0 |
| HPAEC none | 0.0 |
| HPAEC TNF alpha + IL-1 beta | 0.3 |
| Lung fibroblast none | 0.1 |
| Lung fibroblast TNF alpha + IL-1 beta | 0.5 |
| Lung fibroblast IL-4 | 0.1 |
| Lung fibroblast IL-9 | 0.1 |
| Lung fibroblast IL-13 | 0.0 |
| Lung fibroblast IFN gamma | 0.9 |
| Dermal fibroblast CCD1070 rest | 0.2 |
| Dermal fibroblast CCD1070 TNF alpha | 0.1 |
| Dermal fibroblast CCD1070 IL-1 beta | 0.1 |
| Dermal fibroblast IFN gamma | 0.1 |
| Dermal fibroblast IL-4 | 0.1 |
| Dermal Fibroblasts rest | 0.1 |
| Neutrophils TNFa + LPS | 21.2 |
| Neutrophils rest | 2.1 |
| Colon | 0.1 |
| Lung | 0.0 |
| Thymus | 0.0 |
| Kidney | 1.5 |

TABLE AKF
Panel 5 Islet

| | Rel. | Rel. |
|---|---|---|
| | Exp. (%) | Exp. (%) |
| | Ag5581, | Ag5581, |
| | Run | Run |
| Tissue Name | 244908254 | 279370998 |
| 97457_Patient-02go_adipose | 0.0 | 3.1 |
| 97476_Patient-07sk_skeletal | 4.0 | 0.0 |
| muscle | | |
| 97477_Patient-07ut_uterus | 0.0 | 0.0 |
| 97478_Patient-07pl_placenta | 5.1 | 3.3 |
| 99167_Bayer Patient 1 | 3.3 | 0.0 |
| 97482_Patient-08ut_uterus | 4.3 | 0.0 |
| 97483_Patient-08pl_placenta | 0.0 | 7.0 |
| 97486_Patient-09sk_skeletal | 0.0 | 3.1 |
| muscle | | |
| 97487_Patient-09ut_uterus | 0.0 | 0.0 |
| 97488_Patient-09pl_placenta | 0.0 | 0.0 |
| 97492_Patient-10ut_uterus | 0.0 | 0.0 |
| 97493_Patient-10pl_placenta | 0.0 | 3.7 |
| 97495_Patient-11go_adipose | 0.0 | 2.3 |
| 97496_Patient-11sk_skeletal | 18.3 | 1.7 |
| muscle | | |
| 97497_Patient-11ut_uterus | 0.0 | 2.1 |
| 97498_Patient-11pl_placenta | 0.0 | 0.0 |
| 97500_Patient-12go_adipose | 0.0 | 0.0 |
| 97501_Patient-12sk_skeletal | 6.3 | 0.0 |
| muscle | | |
| 97502_Patient-12ut_uterus | 0.0 | 0.0 |
| 97503_Patient-12pl_placenta | 0.0 | 6.6 |
| 94721_Donor 2 U - | 0.0 | 0.0 |
| A_Mesenchymal Stem Cells | | |
| 94722 Donor 2 U - | 0.0 | 0.0 |
| B_Mesenchymal Stem Cells | | |
| 94723_Donor 2 U - | 0.0 | 7.2 |
| C_Mesenchymal Stem Cells | | |
| 94709_Donor 2 AM - A_adipose | 0.0 | 0.0 |
| 94710_Donor 2 AM - B_adipose | 0.0 | 2.1 |
| 94711_Donor 2 AM - C_adipose | 0.0 | 0.0 |
| 94712_Donor 2 AD - A_adipose | 0.0 | 0.0 |
| 94713_Donor 2 AD - B_adipose | 0.0 | 0.0 |
| 94714_Donor 2 AD - | 0.0 | 0.0 |
| C_adipose | | |
| 94742_Donor 3 U - | 0.0 | 0.0 |
| A_Mesenchymal Stem Cells | | |
| 94743_Donor 3 U - | 0.0 | 0.0 |
| B_Mesenchymal Stem Cells | | |
| 94730_Donor 3 AM - A_adipose | 0.0 | 0.0 |
| 94731_Donor 3 AM - B_adipose | 0.0 | 0.0 |
| 94732_Donor 3 AM - C_adipose | 0.0 | 0.0 |
| 94733_Donor 3 AD - A_adipose | 0.0 | 0.0 |
| 94734_Donor 3 AD - B_adipose | 0.0 | 0.0 |
| 94735_Donor 3 AD - C_adipose | 0.0 | 2.9 |
| 77138_Liver_HepG2untreated | 100.0 | 100.0 |
| 73556_Heart_Cardiac stromal | 0.0 | 0.0 |
| cells (primary) | | |
| 81735_Small Intestine | 35.4 | 29.7 |
| 72409_Kidney_Proximal | 0.0 | 0.0 |
| Convoluted Tubule | | |
| 82685_Small | 12.8 | 44.4 |
| intestine_Duodenum | | |
| 90650_Adrenal_Adrenocortical | 0.0 | 0.0 |
| adenoma | | |
| 72410_Kidney_HRCE | 5.5 | 3.7 |
| 72411_Kidney_HRE | 0.0 | 0.0 |
| 73139_Uterus_Uterine smooth | 0.0 | 0.0 |
| muscle cells | | |

TABLE AKG
Panel 5D

| | Rel. Exp. (%) |
|---|---|
| | Ag5581, Run |
| Tissue Name | 244988601 |
| 97457_Patient-02go_adipose | 7.0 |
| 97476_Patient-07sk_skeletal muscle | 0.0 |
| 97477_Patient-07ut_uterus | 0.0 |
| 97478_Patient-07pl_placenta | 3.4 |
| 97481_Patient-08sk_skeletal muscle | 4.2 |
| 97482_Patient-08ut_uterus | 3.0 |
| 97483_Patient-08pl_placenta | 0.0 |
| 97486_Patient-09sk_skeletal muscle | 0.0 |
| 97487_Patient-09ut_uterus | 0.0 |
| 97488_Patient-09pl_placenta | 0.0 |
| 97492_Patient-10ut_uterus | 9.0 |
| 97493_Patient-10pl_placenta | 8.8 |
| 97495_Patient-11go_adipose | 4.9 |
| 97496_Patient-11sk_skeletal muscle | 0.0 |
| 97497_Patient-11ut_uterus | 0.0 |
| 97498_Patient-11pl_placenta | 4.4 |
| 97500_Patient-12go_adipose | 0.0 |
| 97501_Patient-12sk_skeletal muscle | 4.9 |
| 97502_Patient-12ut_uterus | 4.0 |
| 97503_Patient-12pl_placenta | 9.3 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 100.0 |
| 73556_Heart_Cardiac stromal cells (primary) | 0.0 |
| 81735_Small Intestine | 25.0 |
| 72409_Kidney_Proximal Convoluted Tubule | 0.0 |
| 82685_Small intestine_Duodenum | 40.3 |
| 90650_Adrenal_Adrenocortical adenoma | 0.0 |
| 72410_Kidney_HRCE | 3.3 |
| 72411_Kidney_HRE | 0.0 |
| 73139_Uterus_Uterine smooth muscle cells | 0.0 |

TABLE AKH
general oncology screening panel_v_2.4

| | Rel. Exp. (%) |
|---|---|
| | Ag5581, Run |
| Tissue Name | 260268963 |
| Colon cancer 1 | 17.7 |
| Colon cancer NAT 1 | 0.0 |
| Colon cancer 2 | 15.4 |
| Colon cancer NAT 2 | 8.2 |
| Colon cancer 3 | 13.2 |
| Colon cancer NAT 3 | 6.1 |
| Colon malignant cancer 4 | 44.1 |
| Colon normal adjacent tissue 4 | 2.2 |
| Lung cancer 1 | 25.0 |
| Lung NAT 1 | 3.3 |
| Lung cancer 2 | 32.8 |
| Lung NAT 2 | 6.7 |
| Squamous cell carcinoma 3 | 25.0 |
| Lung NAT 3 | 3.2 |
| metastatic melanoma 1 | 1.5 |
| Melanoma 2 | 1.2 |
| Melanoma 3 | 0.0 |
| metastatic melanoma 4 | 2.6 |
| metastatic melanoma 5 | 14.2 |
| Bladder cancer 1 | 6.2 |
| Bladder cancer NAT 1 | 0.0 |
| Bladder cancer 2 | 0.0 |
| Bladder cancer NAT 2 | 0.0 |
| Bladder cancer NAT 3 | 0.0 |
| Bladder cancer NAT 4 | 0.0 |
| Prostate adenocarcinoma 1 | 2.7 |
| Prostate adenocarcinoma 2 | 0.0 |
| Prostate adenocarcinoma 3 | 0.0 |
| Prostate adenocarcinoma 4 | 2.2 |
| Prostate cancer NAT 5 | 0.0 |
| Prostate adenocarcinoma 6 | 0.0 |
| Prostate adenocarcinoma 7 | 3.3 |
| Prostate adenocarcinoma 8 | 0.0 |
| Prostate adenocarcinoma 9 | 0.0 |
| Prostate cancer NAT 10 | 0.0 |
| Kidney cancer 1 | 32.5 |
| Kidney NAT 1 | 2.9 |
| Kidney cancer 2 | 12.4 |
| Kidney NAT 2 | 10.7 |
| Kidney cancer 3 | 15.9 |
| Kidney NAT 3 | 16.4 |
| Kidney cancer 4 | 12.9 |
| Kidney NAT 4 | 100.0 |

AI_comprehensive panel_v1.0 Summary: Ag5581 Two experiments with the same probe and primer set show detectable expression of this gene limited to a sample of normal tissue adjacent to ulcerative colitis (CTs=33.5-34.5) and a sample derived from RA synovial fluid.

General_screening_panel_v1.5 Summary: Ag5581 Highest expression is seen in fetal liver (CT=30.6). In addition, this gene is expressed at much higher levels in fetal liver tissue when compared to expression in the adult counterpart (CT=34). Thus, expression of this gene may be used to differentiate between the fetal and adult source of this tissue.

General_screening_panel_v1.6 Summary: Ag5581 Highest expression is seen in fetal liver (CT=30.3). Overall, expression is in agreement with Panel 1.5. Please see that panel for further discussion of expression and utility of this gene.

Panel 4.1D Summary: Ag5581 Highest expression is seen in LPS treated monocytes (CT=27.4). Moderate levels of expression are seen in TFN-a/LPS treated neutropils and PMA/ionomycin treated LAKs. Low but significant levels of expression are seen in macrophages. Upon activation with pathogens such as LPS, monocytes contribute to the innate and specific immunity by migrating to the site of tissue injury and releasing inflammatory cytokines. This release contributes to the inflammation process. Therefore expression of this gene could be used as a marker of activated monocytes. Furthermore, modulation of the expression of the protein encoded by this transcript may prevent the recruitment of monocytes and the initiation of the inflammatory process, and reduce the symptoms of patients suffering from autoimmune and inflammatory diseases such as asthma, allergies, inflammatory bowel disease, lupus erythematosus, or rheumatoid arthritis.

Panel 5 Islet Summary: Ag5581 Two experiments with the same probe and primer set show detectable expression of this gene limited to a liver cancer cell line sample (CTs=33.5-34.5). This expression is in agreement with expression seen in Panels 1.5 and 1.6.

Panel 5D Summary: Ag5581 Expression of this gene limited to a liver cancer cell line sample (CT=34). This expression is in agreement with expression seen in Panels 1.5 and 1.6.

General oncology screening panel_v_{—}2.4 Summary: Ag5581 Highest expression is seen in a kidney sample (CT=32). In addition, this gene is more highly expressed in lung and colon cancer than in the corresponding normal adjacent tissue. Thus, expression of this gene could be used as a marker of these cancers. Furthemore, therapeutic modulation of the expression or function of this gene product may be useful in the treatment of lung and colon cancer.

AL. CG160131-04: FL_{—}1_{—}552 Glycerol Kinase.

Expression of gene CG160131-04 was assessed using the primer-probe set Ag7439, described in Table ALA. Results of the RTQ-PCR runs are shown in Tables ALB and ALC. Please note that CG160131-04 represents a full-length physical clone.

TABLE ALA
Probe Name Ag7439

| | | | Start | SEQ ID |
|---|---|---|---|---|
| Primers | Sequences | Length | Position | No |
| Forward | 5′-agcacatttgtcccaccaat-3′ | 20 | 774 | 585 |
| Probe | TET-5′-cacccagatattggcacaccttccaa-3′-TAMRA | 26 | 815 | 586 |
| Reverse | 5′-atgaaaatctctcatagcgtgaa-3′ | 23 | 851 | 587 |

TABLE ALB
AI_comprehensive panel_v1.0

| | Rel. Exp. (%) |
|---|---|
| | Ag7439, Run |
| Tissue Name | 311756513 |
| 110967 COPD-F | 19.1 |
| 110980 COPD-F | 18.3 |
| 110968 COPD-M | 16.5 |
| 110977 COPD-M | 68.8 |
| 110989 Emphysema-F | 54.3 |
| 110992 Emphysema-F | 8.2 |
| 110993 Emphysema-F | 35.8 |
| 110994 Emphysema-F | 13.5 |
| 110995 Emphysema-F | 29.3 |
| 110996 Emphysema-F | 2.1 |
| 110997 Asthma-M | 2.5 |
| 111001 Asthma-F | 32.8 |
| 111002 Asthma-F | 26.2 |
| 111003 Atopic Asthma-F | 30.6 |
| 111004 Atopic Asthma-F | 18.6 |
| 111005 Atopic Asthma-F | 17.6 |
| 111006 Atopic Asthma-F | 4.1 |
| 111417 Allergy-M | 12.2 |
| 112347 Allergy-M | 0.0 |
| 112349 Normal Lung-F | 0.0 |
| 112357 Normal Lung-F | 52.1 |
| 112354 Normal Lung-M | 27.7 |
| 112374 Crohns-F | 27.2 |
| 112389 Match Control Crohns-F | 8.6 |
| 112375 Crohns-F | 20.7 |
| 112732 Match Control Crohns-F | 4.7 |
| 112725 Crohns-M | 5.4 |
| 112387 Match Control Crohns-M | 12.6 |
| 112378 Crohns-M | 0.0 |
| 112390 Match Control Crohns-M | 56.3 |
| 112726 Crohns-M | 21.2 |
| 112731 Match Control Crohns-M | 20.0 |
| 112380 Ulcer Col-F | 31.9 |
| 112734 Match Control Ulcer Col-F | 15.3 |
| 112384 Ulcer Col-F | 43.2 |
| 112737 Match Control Ulcer Col-F | 5.5 |
| 112386 Ulcer Col-F | 15.2 |
| 112738 Match Control Ulcer Col-F | 5.6 |
| 112381 Ulcer Col-M | 0.1 |
| 112735 Match Control Ulcer Col-M | 3.0 |
| 112382 Ulcer Col-M | 18.4 |
| 112394 Match Control Ulcer Col-M | 8.9 |
| 112383 Ulcer Col-M | 24.7 |
| 112736 Match Control Ulcer Col-M | 6.3 |
| 112423 Psoriasis-F | 21.8 |
| 112427 Match Control Psoriasis-F | 100.0 |
| 112418 Psoriasis-M | 23.5 |
| 112723 Match Control Psoriasis-M | 21.2 |
| 112419 Psoriasis-M | 43.8 |
| 112424 Match Control Psoriasis-M | 23.2 |
| 112420 Psoriasis-M | 79.6 |
| 112425 Match Control Psoriasis-M | 82.9 |
| 104689 (MF) OA Bone-Backus | 20.0 |
| 104690 (MF) Adj “Normal” Bone-Backus | 24.0 |
| 104691 (MF) OA Synovium-Backus | 71.7 |
| 104692 (BA) OA Cartilage-Backus | 0.0 |
| 104694 (BA) OA Bone-Backus | 27.2 |
| 104695 (BA) Adj “Normal” Bone-Backus | 24.3 |
| 104696 (BA) OA Synovium-Backus | 57.4 |
| 104700 (SS) OA Bone-Backus | 16.2 |
| 104701 (SS) Adj “Normal” Bone-Backus | 18.2 |
| 104702 (SS) OA Synovium-Backus | 39.8 |
| 117093 OA Cartilage Rep7 | 31.4 |
| 112672 OA Bone5 | 77.4 |
| 112673 OA Synovium5 | 35.8 |
| 112674 OA Synovial Fluid cells5 | 47.0 |
| 117100 OA Cartilage Rep14 | 8.4 |
| 112756 OA Bone9 | 69.3 |
| 112757 OA Synovium9 | 20.6 |
| 112758 OA Synovial Fluid Cells9 | 9.2 |
| 117125 RA Cartilage Rep2 | 13.4 |
| 113492 Bone2 RA | 18.7 |
| 113493 Synovium2 RA | 4.2 |
| 113494 Syn Fluid Cells RA | 6.8 |
| 113499 Cartilage4 RA | 7.7 |
| 113500 Bone4 RA | 11.0 |
| 113501 Synovium4 RA | 9.2 |
| 113502 Syn Fluid Cells4 RA | 4.5 |
| 113495 Cartilage3 RA | 6.3 |
| 113496 Bone3 RA | 7.2 |
| 113497 Synovium3 RA | 4.6 |
| 113498 Syn Fluid Cells3 RA | 10.3 |
| 117106 Normal Cartilage Rep20 | 2.8 |
| 113663 Bone3 Normal | 0.0 |
| 113664 Synovium3 Normal | 0.0 |
| 113665 Syn Fluid Cells3 Normal | 0.0 |
| 117107 Normal Cartilage Rep22 | 5.8 |
| 113667 Bone4 Normal | 32.5 |
| 113668 Synovium4 Normal | 21.8 |
| 113669 Syn Fluid Cells4 Normal | 43.2 |

TABLE ALC
Panel 4.1D

| | Rel. Exp. (%) |
|---|---|
| | Ag7439, Run |
| Tissue Name | 305901963 |
| Secondary Th1 act | 1.9 |
| Secondary Th2 act | 1.7 |
| Secondary Tr1 act | 1.0 |
| Secondary Th1 rest | 0.0 |
| Secondary Th2 rest | 0.3 |
| Secondary Tr1 rest | 0.0 |
| Primary Th1 act | 0.1 |
| Primary Th2 act | 1.2 |
| Primary Tr1 act | 2.0 |
| Primary Th1 rest | 0.0 |
| Primary Th2 rest | 0.0 |
| Primary Tr1 rest | 0.0 |
| CD45RA CD4 lymphocyte act | 5.2 |
| CD45RO CD4 lymphocyte act | 1.1 |
| CD8 lymphocyte act | 0.0 |
| Secondary CD8 lymphocyte rest | 4.0 |
| Secondary CD8 lymphocyte act | 0.0 |
| CD4 lymphocyte none | 0.0 |
| 2ry Th1/Th2/Tr1_anti-CD95 CH11 | 0.5 |
| LAK cells rest | 0.3 |
| LAK cells IL-2 | 0.2 |
| LAK cells IL-2 + IL-12 | 0.0 |
| LAK cells IL-2 + IFN gamma | 0.2 |
| LAK cells IL-2 + IL-18 | 0.2 |
| LAK cells PMA/ionomycin | 14.6 |
| NK Cells IL-2 rest | 0.9 |
| Two Way MLR 3 day | 3.1 |
| Two Way MLR 5 day | 0.4 |
| Two Way MLR 7 day | 0.1 |
| PBMC rest | 0.1 |
| PBMC PWM | 0.9 |
| PBMC PHA-L | 0.5 |
| Ramos (B cell) none | 0.1 |
| Ramos (B cell) ionomycin | 0.1 |
| B lymphocytes PWM | 0.4 |
| B lymphocytes CD40L and IL-4 | 0.2 |
| EOL-1 dbcAMP | 1.9 |
| EOL-1 dbcAMP PMA/ionomycin | 0.1 |
| Dendritic cells none | 1.7 |
| Dendritic cells LPS | 1.2 |
| Dendritic cells anti-CD40 | 0.3 |
| Monocytes rest | 0.1 |
| Monocytes LPS | 31.2 |
| Macrophages rest | 0.4 |
| Macrophages LPS | 0.8 |
| HUVEC none | 0.6 |
| HUVEC starved | 2.8 |
| HUVEC IL-1beta | 2.2 |
| HUVEC IFN gamma | 1.8 |
| HUVEC TNF alpha + IFN gamma | 0.6 |
| HUVEC TNF alpha + IL4 | 1.7 |
| HUVEC IL-11 | 0.6 |
| Lung Microvascular EC none | 3.4 |
| Lung Microvascular EC TNFalpha + IL-1beta | 3.2 |
| Microvascular Dermal EC none | 0.1 |
| Microsvasular Dermal EC TNFalpha + IL-1beta | 1.3 |
| Bronchial epithelium TNFalpha + IL1beta | 0.4 |
| Small airway epithelium none | 0.3 |
| Small airway epithelium TNFalpha + IL-1beta | 0.0 |
| Coronery artery SMC rest | 2.2 |
| Coronery artery SMC TNFalpha + IL-1beta | 3.6 |
| Astrocytes rest | 0.0 |
| Astrocytes TNFalpha + IL-1beta | 0.8 |
| KU-812 (Basophil) rest | 0.3 |
| KU-812 (Basophil) PMA/ionomycin | 0.1 |
| CCD1106 (Keratinocytes) none | 0.0 |
| CCD1106 (Keratinocytes) TNFalpha + IL-1beta | 0.0 |
| Liver cirrhosis | 0.0 |
| NCI-H292 none | 0.0 |
| NCI-H292 IL-4 | 0.2 |
| NCI-H292 IL-9 | 0.0 |
| NCI-H292 IL-13 | 1.0 |
| NCI-H292 IFN gamma | 0.0 |
| HPAEC none | 0.8 |
| HPAEC TNF alpha + IL-1 beta | 8.8 |
| Lung fibroblast none | 24.5 |
| Lung fibroblast TNF alpha + IL-1 beta | 43.2 |
| Lung fibroblast IL-4 | 14.5 |
| Lung fibroblast IL-9 | 21.8 |
| Lung fibroblast IL-13 | 12.9 |
| Lung fibroblast IFN gamma | 100.0 |
| Dermal fibroblast CCD1070 rest | 6.1 |
| Dermal fibroblast CCD1070 TNF alpha | 11.6 |
| Dermal fibroblast CCD1070 IL-1 beta | 11.0 |
| Dermal fibroblast IFN gamma | 7.2 |
| Dermal fibroblast IL-4 | 3.8 |
| Dermal Fibroblasts rest | 2.6 |
| Neutrophils TNFa + LPS | 2.6 |
| Neutrophils rest | 1.6 |
| Colon | 0.0 |
| Lung | 0.3 |
| Thymus | 0.2 |
| Kidney | 1.1 |

AI_comprehensive panel_v1.0 Summary: Ag7439 Highest expression is seen in normal tissue adjacent to psoriasis (CT=29.8). In addition, moderate to low levels of expression are seen in many samples on this panel. Thus, this gene product may be involved in autoimmune disease.

CNS_neurodegeneration_v1.0 Summary: Ag7439 Results from one experiment with this gene are not included. The amp plot indicates that there were experimental difficulties with this run.

Panel 4.1D Summary: Ag7439 Highest expression is seen in a sample of IFN gama lung derived fibroblasts (CT=29). Low but significant levels of expression are also seen in clusters of samples derived from lung and dermal fibroblasts. Thus, this gene product may be involved in inflammatory processes of the lung and skin, including psoriasis, asthma, emphysema, and allergy.

Panel 5 Islet Summary: Ag7439 Expression of this gene is low/undetectable in all samples on this panel (CTs>35). (Data not shown.)

AM. CG166282-01: CHK1-Variant.

Expression of gene CG166282-01 was assessed using the primer-probe set Ag5448, described in Table AMA. Results of the RTQ-PCR runs are shown in Tables AMB, AMC and AMD.

TABLE AMA
Probe Name Ag5448

| | | | Start | SEQ ID |
|---|---|---|---|---|
| Primers | Sequences | Length | Position | No |
| Forward | 5′-tgtatgaatcagggtgatggat-3′ | 22 | 1256 | 588 |
| Probe | TET-5′-tcttcaggaagtgtctcttgaactcca-3′-TAMRA | 27 | 1278 | 589 |
| Reverse | 5′-ctggctgctcacaatatcaatc-3′ | 22 | 1318 | 590 |

TABLE AMB
General_screening_panel_v1.5

| | Rel. | Rel. |
|---|---|---|
| | Exp. (%) | Exp. (%) |
| | Ag5448, | Ag5448, |
| | Run | Run |
| Tissue Name | 237375423 | 247291071 |
| Adipose | 0.2 | 0.0 |
| Melanoma* Hs688(A).T | 6.7 | 3.1 |
| Melanoma* Hs688(B).T | 5.0 | 4.3 |
| Melanoma* M14 | 25.5 | 18.8 |
| Melanoma* LOXIMVI | 28.7 | 22.5 |
| Melanoma* SK-MEL-5 | 17.3 | 12.2 |
| Squamous cell carcinoma SCC-4 | 5.6 | 5.6 |
| Testis Pool | 0.5 | 2.2 |
| Prostate ca. * (bone met) PC-3 | 11.7 | 9.3 |
| Prostate Pool | 0.0 | 0.0 |
| Placenta | 0.0 | 1.4 |
| Uterus Pool | 0.3 | 0.0 |
| Ovarian ca. OVCAR-3 | 10.2 | 6.5 |
| Ovarian ca. SK-OV-3 | 32.3 | 35.8 |
| Ovarian ca. OVCAR-4 | 22.8 | 16.5 |
| Ovarian ca. OVCAR-5 | 12.6 | 5.5 |
| Ovarian ca. IGROV-1 | 8.7 | 9.5 |
| Ovarian ca. OVCAR-8 | 10.4 | 9.0 |
| Ovary | 0.2 | 0.0 |
| Breast ca. MCF-7 | 4.2 | 5.8 |
| Breast ca. MDA-MB-231 | 56.3 | 45.7 |
| Breast ca. BT 549 | 27.9 | 16.7 |
| Breast ca. T47D | 17.6 | 15.0 |
| Breast ca. MDA-N | 12.9 | 14.8 |
| Breast Pool | 0.1 | 0.0 |
| Trachea | 0.8 | 0.0 |
| Lung | 0.0 | 0.0 |
| Fetal Lung | 2.2 | 0.0 |
| Lung ca. NCI-N417 | 8.1 | 7.2 |
| Lung ca. LX-1 | 20.0 | 8.8 |
| Lung ca. NCI-H146 | 9.7 | 9.3 |
| Lung ca. SHP-77 | 22.5 | 16.0 |
| Lung ca. A549 | 18.7 | 10.5 |
| Lung ca. NCI-H526 | 30.1 | 26.4 |
| Lung ca. NCI-H23 | 16.5 | 12.3 |
| Lung ca. NCI-H460 | 4.5 | 1.2 |
| Lung ca. HOP-62 | 2.6 | 2.2 |
| Lung ca. NCI-H522 | 32.8 | 31.2 |
| Liver | 0.0 | 0.0 |
| Fetal Liver | 4.5 | 6.9 |
| Liver ca. HepG2 | 5.8 | 4.1 |
| Kidney Pool | 0.4 | 0.0 |
| Fetal Kidney | 6.2 | 7.4 |
| Renal ca. 786-0 | 12.5 | 12.5 |
| Renal ca. A498 | 2.0 | 3.5 |
| Renal ca. ACHN | 6.6 | 3.2 |
| Renal ca. UO-31 | 23.3 | 21.3 |
| Renal ca. TK-10 | 8.2 | 5.0 |
| Bladder | 3.2 | 0.0 |
| Gastric ca. (liver met.) NCI-N87 | 8.0 | 8.0 |
| Gastric ca. KATO III | 100.0 | 100.0 |
| Colon ca. SW-948 | 7.7 | 7.9 |
| Colon ca. SW480 | 62.0 | 46.0 |
| Colon ca. * (SW480 met) SW620 | 32.8 | 31.9 |
| Colon ca. HT29 | 18.6 | 5.1 |
| Colon ca. HCT-116 | 33.9 | 39.5 |
| Colon ca. CaCo-2 | 27.0 | 19.3 |
| Colon cancer tissue | 5.5 | 4.4 |
| Colon ca. SW1116 | 4.1 | 5.3 |
| Colon ca. Colo-205 | 8.8 | 8.4 |
| Colon ca. SW-48 | 13.6 | 8.0 |
| Colon Pool | 0.4 | 0.0 |
| Small Intestine Pool | 1.0 | 0.0 |
| Stomach Pool | 0.6 | 0.0 |
| Bone Marrow Pool | 0.0 | 0.0 |
| Fetal Heart | 2.4 | 2.6 |
| Heart Pool | 0.6 | 0.0 |
| Lymph Node Pool | 0.0 | 0.0 |
| Fetal Skeletal Muscle | 0.3 | 0.0 |
| Skeletal Muscle Pool | 0.0 | 0.0 |
| Spleen Pool | 0.2 | 1.9 |
| Thymus Pool | 2.1 | 2.4 |
| CNS cancer (glio/astro) U87-MG | 14.2 | 9.2 |
| CNS cancer (glio/astro) U-118-MG | 44.1 | 47.3 |
| CNS cancer (neuro; met) SK-N-AS | 8.3 | 12.2 |
| CNS cancer (astro) SF-539 | 6.4 | 8.5 |
| CNS cancer (astro) SNB-75 | 29.9 | 30.6 |
| CNS cancer (glio) SNB-19 | 4.4 | 5.4 |
| CNS cancer (glio) SF-295 | 8.7 | 4.2 |
| Brain (Amygdala) Pool | 0.0 | 0.0 |
| Brain (cerebellum) | 0.5 | 0.0 |
| Brain (fetal) | 2.7 | 1.2 |
| Brain (Hippocampus) Pool | 0.6 | 0.0 |
| Cerebral Cortex Pool | 0.0 | 0.0 |
| Brain (Substantia nigra) Pool | 0.0 | 0.0 |
| Brain (Thalamus) Pool | 0.4 | 0.0 |
| Brain (whole) | 0.0 | 0.0 |
| Spinal Cord Pool | 0.1 | 0.0 |
| Adrenal Gland | 0.0 | 0.0 |
| Pituitary gland Pool | 0.2 | 0.0 |
| Salivary Gland | 0.4 | 0.0 |
| Thyroid (female) | 0.2 | 0.0 |
| Pancreatic ca. CAPAN2 | 40.1 | 48.0 |
| Pancreas Pool | 1.4 | 0.0 |

TABLE AMC
Panel 4.1D

| | Rel. Exp. (%) |
|---|---|
| | Ag5448, Run |
| Tissue Name | 237371903 |
| Secondary Th1 act | 88.9 |
| Secondary Th2 act | 100.0 |
| Secondary Tr1 act | 16.6 |
| Secondary Th1 rest | 0.0 |
| Secondary Th2 rest | 0.0 |
| Secondary Tr1 rest | 0.0 |
| Primary Th1 act | 0.0 |
| Primary Th2 act | 47.3 |
| Primary Tr1 act | 50.7 |
| Primary Th1 rest | 1.5 |
| Primary Th2 rest | 7.9 |
| Primary Tr1 rest | 0.0 |
| CD45RA CD4 lymphocyte act | 41.5 |
| CD45RO CD4 lymphocyte act | 77.9 |
| CD8 lymphocyte act | 11.0 |
| Secondary CD8 lymphocyte rest | 99.3 |
| Secondary CD8 lymphocyte act | 13.3 |
| CD4 lymphocyte none | 0.0 |
| 2ry Th1/Th2/Tr1_anti-CD95 CH11 | 0.0 |
| LAK cells rest | 1.6 |
| LAK cells IL-2 | 15.4 |
| LAK cells IL-2 + IL-12 | 2.0 |
| LAK cells IL-2 + IFN gamma | 17.6 |
| LAK cells IL-2 + IL-18 | 5.6 |
| LAK cells PMA/ionomycin | 13.3 |
| NK Cells IL-2 rest | 35.1 |
| Two Way MLR 3 day | 1.2 |
| Two Way MLR 5 day | 6.3 |
| Two Way MLR 7 day | 2.2 |
| PBMC rest | 0.0 |
| PBMC PWM | 14.6 |
| PBMC PHA-L | 7.3 |
| Ramos (B cell) none | 4.0 |
| Ramos (B cell) ionomycin | 41.5 |
| B lymphocytes PWM | 45.4 |
| B lymphocytes CD40L and IL-4 | 27.0 |
| EOL-1 dbcAMP | 74.2 |
| EOL-1 dbcAMP PMA/ionomycin | 2.3 |
| Dendritic cells none | 0.0 |
| Dendritic cells LPS | 0.0 |
| Dendritic cells anti-CD40 | 0.0 |
| Monocytes rest | 0.0 |
| Monocytes LPS | 11.3 |
| Macrophages rest | 0.0 |
| Macrophages LPS | 0.0 |
| HUVEC none | 26.4 |
| HUVEC starved | 24.0 |
| HUVEC IL-1beta | 44.1 |
| HUVEC IFN gamma | 17.1 |
| HUVEC TNF alpha + IFN gamma | 2.2 |
| HUVEC TNF alpha + IL4 | 1.6 |
| HUVEC IL-11 | 15.5 |
| Lung Microvascular EC none | 11.9 |
| Lung Microvascular EC TNFalpha + IL-1beta | 7.8 |
| Microvascular Dermal EC none | 3.8 |
| Microsvasular Dermal EC TNFalpha + IL-1beta | 3.7 |
| Bronchial epithelium TNFalpha + ILlbeta | 3.1 |
| Small airway epithelium none | 12.7 |
| Small airway epithelium TNFalpha + IL-1beta | 19.6 |
| Coronery artery SMC rest | 7.5 |
| Coronery artery SMC TNFalpha + IL-1beta | 2.2 |
| Astrocytes rest | 1.4 |
| Astrocytes TNFalpha + IL-1beta | 0.0 |
| KU-812 (Basophil) rest | 34.6 |
| KU-812 (Basophil) PMA/ionomycin | 45.1 |
| CCD1106 (Keratinocytes) none | 24.7 |
| CCD1106 (Keratinocytes) TNFalpha + IL-1beta | 10.5 |
| Liver cirrhosis | 0.0 |
| NCI-H292 none | 13.7 |
| NCI-H292 IL-4 | 38.7 |
| NCI-H292 IL-9 | 23.7 |
| NCI-H292 IL-13 | 41.2 |
| NCI-H292 IFN gamma | 22.8 |
| HPAEC none | 4.4 |
| HPAEC TNF alpha + IL-1 beta | 11.2 |
| Lung fibroblast none | 5.2 |
| Lung fibroblast TNF alpha + IL-1 beta | 7.1 |
| Lung fibroblast IL-4 | 1.6 |
| Lung fibroblast IL-9 | 0.0 |
| Lung fibroblast IL-13 | 0.0 |
| Lung fibroblast IFN gamma | 3.5 |
| Dermal fibroblast CCD1070 rest | 33.9 |
| Dermal fibroblast CCD1070 TNF alpha | 59.0 |
| Dermal fibroblast CCD1070 IL-1 beta | 23.8 |
| Dermal fibroblast IFN gamma | 22.4 |
| Dermal fibroblast IL-4 | 31.0 |
| Dermal Fibroblasts rest | 16.7 |
| Neutrophils TNFa + LPS | 0.0 |
| Neutrophils rest | 0.0 |
| Colon | 0.0 |
| Lung | 0.0 |
| Thymus | 0.0 |
| Kidney | 0.0 |

TABLE AMD
general oncology screening panel_v_2.4

| | Rel. Exp. (%) |
|---|---|
| | Ag5448, Run |
| Tissue Name | 260285334 |
| Colon cancer 1 | 15.9 |
| Colon NAT 1 | 3.4 |
| Colon cancer 2 | 26.8 |
| Colon NAT 2 | 15.6 |
| Colon cancer 3 | 51.8 |
| Colon NAT 3 | 3.6 |
| Colon malignant cancer 4 | 100.0 |
| Colon NAT 4 | 4.0 |
| Lung cancer 1 | 8.3 |
| Lung NAT 1 | 0.0 |
| Lung cancer 2 | 33.9 |
| Lung NAT 2 | 0.0 |
| Squamous cell carcinoma 3 | 15.5 |
| Lung NAT 3 | 0.0 |
| Metastatic melanoma 1 | 0.0 |
| Melanoma 2 | 0.0 |
| Melanoma 3 | 0.0 |
| Metastatic melanoma 4 | 5.1 |
| Metastatic melanoma 5 | 3.8 |
| Bladder cancer 1 | 0.0 |
| Bladder NAT 1 | 0.0 |
| Bladder cancer 2 | 4.4 |
| Bladder NAT 2 | 0.0 |
| Bladder NAT 3 | 0.0 |
| Bladder NAT 4 | 0.0 |
| Prostate adenocarcinoma 1 | 0.0 |
| Prostate adenocarcinoma 2 | 0.0 |
| Prostate adenocarcinoma 3 | 0.0 |
| Prostate adenocarcinoma 4 | 3.2 |
| Prostate NAT 5 | 0.0 |
| Prostate adenocarcinoma 6 | 0.0 |
| Prostate adenocarcinoma 7 | 0.0 |
| Prostate adenocarcinoma 8 | 0.0 |
| Prostate adenocarcinoma 9 | 0.0 |
| Prostate NAT 10 | 0.0 |
| Kidney cancer 1 | 0.0 |
| Kidney NAT 1 | 0.0 |
| Kidney cancer 2 | 15.9 |
| Kidney NAT 2 | 0.0 |
| Kidney cancer 3 | 5.2 |
| Kidney NAT 3 | 0.0 |
| Kidney cancer 4 | 0.0 |
| Kidney NAT 4 | 0.0 |

AI_comprehensive panel_v1.0 Summary: Ag5448 The amp plot indicates that there were experimental difficulties with this run; therefore, no conclusions can be drawn from this data. (Data not shown).

General_screening_panel_v1.5 Summary: Ag5448 Two experiments with same probe-primer sets are in excellent agreement, with highest expression of this gene detected in gastric cancer KATO III cell line (CTs=30-33). Moderate to low levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Oncology_cell_line_screening_panel_v3.2 Summary: Ag5448 The amp plot indicates that there were experimental difficulties with this run; therefore, no conclusions can be drawn from this data. (Data not shown).

Panel 4.1D Summary: Ag5448 Highest expression of this gene is detected in activated secondary Th2 cells (CT=33). Low expression of this gene is detected in activated polarized T cells, resting IL-2 treated NK cells, activated Ramos B cells and B lymphocytes, eosinophils, activated HUVEC cells and NCI-H292 cells, basophils and TNF alpha stimulated dermal fibroblasts. Therefore, therapeutic modulation of this gene product may ameliorate symptoms/conditions associated with autoimmune and inflammatory disorders including psoriasis, allergy, asthma, inflammatory bowel disease, rheumatoid arthritis and osteoarthritis.

General oncology screening panel_v_{—}2.4 Summary: Ag5448 Highest expression of this gene malignant colon cancer (CT=34.4). Higher expression of this gene is associated with the colon cancer as compared to adjacent control tissue. Therefore, expression of this gene may be used as diagnostic marker to detect colon cancer and also, therapeutic modulation of this gene or its protein product may be useful in the treatement of colon cancer.

AN. CG170739-01: Pendrin.

Expression of gene CG170739-01 was assessed using the primer-probe set Ag6134, described in Table ANA.

TABLE ANA
Probe Name Ag6134

| | | | Start | SEQ ID |
|---|---|---|---|---|
| Primers | Sequences | Length | Position | No |
| Forward | 5′-cgctgcaaggaccttttc-3′ | 18 | 1931 | 591 |
| Probe | TET-5′tgctcagaacaacagatcccaccatt-3′-TAMRA | 26 | 1892 | 592 |
| Reverse | 5′-tgctggatacgagaaagtgttc-3′ | 22 | 1859 | 593 |

AI_comprehensive panel_v1.0 Summary: Ag6134 Expression of this gene is low/undetectable (CTs>35) across all of the samples on this panel (data not shown). The amp plot indicates that there is a high probability of a probe failure.

General_screening_panel_v1.5 Summary: Ag6134 Expression of this gene is low/undetectable (CTs>35) across all of the samples on this panel (data not shown). The amp plot indicates that there is a high probability of a probe failure.

Panel 4.1D Summary: Ag6134 Expression of this gene is low/undetectable (CTs>35) across all of the samples on this panel (data not shown). The amp plot indicates that there is a high probability of a probe failure.

AO. CG51213-07: CG51213-(13-364).

Expression of gene CG51213-07 was assessed using the primer-probe sets Ag1425, Ag813, Ag871 and Ag924, described in Tables AOA, AOB, AOC and AOD. Results of the RTQ-PCR runs are shown in Tables AOE, AOF, AOG, AOH, AOI, AOJ and AOK.

TABLE AOA
Probe Name Ag1425

| | | | Start | SEQ ID |
|---|---|---|---|---|
| Primers | Sequences | Length | Position | No |
| Forward | 5′-ggacttcagagaagtgcagtgt-3′ | 22 | 549 | 594 |
| Probe | TET-5′-ctgaatttgacagcatccctttccgt-3′-TAMRA | 26 | 572 | 595 |
| Reverse | 5′-cggtacgttttccacttgtaga-3′ | 22 | 605 | 596 |

TABLE AOB
Probe Name Ag813

| | | | Start | SEQ ID |
|---|---|---|---|---|
| Primers | Sequences | Length | Position | No |
| Forward | 5′-tgtagaatttcccacggaaag-3′ | 21 | 590 | 597 |
| Probe | TET-5′-cactgcacttctctgaagtcctggga-3′-TAMRA | 26 | 544 | 598 |
| Reverse | 5′-ctgcaacacggatgactgt-3′ | 19 | 516 | 599 |

TABLE AOC
Probe Name Ag871

| | | | Start | SEQ ID |
|---|---|---|---|---|
| Primers | Sequences | Length | Position | No |
| Forward | 5′-tctagctgggaccacctttc-3′ | 20 | 1041 | 600 |
| Probe | TET-5′-cagaccaggtccagagcctcgaag-3′-TAMRA | 24 | 1076 | 601 |
| Reverse | 5′-acgatgagagatgcattaatcg-3′ | 22 | 1109 | 602 |

TABLE AOD
Probe Name Ag924

| | | | Start | SEQ ID |
|---|---|---|---|---|
| Primers | Sequences | Length | Position | No |
| Forward | 5′-ggacttcagagaagtgcagtgt-3′ | 22 | 549 | 603 |
| Probe | TET-5′-ctgaatttgacagcatccctttccgt-3′-TAMRA | 26 | 572 | 604 |
| Reverse | 5′-cggtacgttttccacttgtaga-3′ | 22 | 605 | 605 |

TABLE AOE
AI_comprehensive panel_v1.0

| | Rel. | Rel. |
|---|---|---|
| | Exp. (%) | Exp. (%) |
| | Ag813, | Ag813, |
| | Run | Run |
| Tissue Name | 234222162 | 246953625 |
| 110967 COPD-F | 5.4 | 8.8 |
| 110980 COPD-F | 5.9 | 9.2 |
| 110968 COPD-M | 12.9 | 11.9 |
| 110977 COPD-M | 18.8 | 25.7 |
| 110989 Emphysema-F | 19.3 | 26.4 |
| 110992 Emphysema-F | 13.5 | 30.8 |
| 110993 Emphysema-F | 10.5 | 13.2 |
| 110994 Emphysema-F | 10.4 | 7.3 |
| 110995 Emphysema-F | 25.5 | 25.9 |
| 110996 Emphysema-F | 3.7 | 6.5 |
| 110997 Asthma-M | 2.5 | 2.4 |
| 111001 Asthma-F | 16.3 | 21.0 |
| 111002 Asthma-F | 24.0 | 22.1 |
| 111003 Atopic Asthma-F | 14.9 | 35.4 |
| 111004 Atopic Asthma-F | 31.6 | 47.0 |
| 111005 Atopic Asthma-F | 18.4 | 20.2 |
| 111006 Atopic Asthma-F | 2.6 | 5.6 |
| 111417 Allergy-M | 13.4 | 8.5 |
| 112347 Allergy-M | 0.0 | 0.0 |
| 112349 Normal Lung-F | 0.0 | 0.0 |
| 112357 Normal Lung-F | 15.5 | 16.4 |
| 112354 Normal Lung-M | 3.7 | 1.5 |
| 112374 Crohns-F | 16.6 | 21.6 |
| 112389 Match Control Crohns-F | 10.3 | 6.3 |
| 112375 Crohns-F | 0.0 | 32.8 |
| 112732 Match Control Crohns-F | 10.4 | 9.7 |
| 112725 Crohns-M | 2.2 | 0.8 |
| 112387 Match Control Crohns-M | 8.4 | 10.5 |
| 112378 Crohns-M | 0.0 | 0.0 |
| 112390 Match Control Crohns-M | 38.7 | 38.2 |
| 112726 Crohns-M | 27.4 | 22.8 |
| 112731 Match Control Crohns-M | 7.6 | 13.6 |
| 112380 Ulcer Col-F | 15.9 | 20.4 |
| 112734 Match Control Ulcer Col-F | 13.5 | 26.4 |
| 112384 Ulcer Col-F | 21.6 | 18.8 |
| 112737 Match Control Ulcer Col-F | 5.6 | 5.8 |
| 112386 Ulcer Col-F | 0.7 | 1.1 |
| 112738 Match Control Ulcer Col-F | 3.0 | 4.3 |
| 112381 Ulcer Col-M | 0.0 | 0.1 |
| 112735 Match Control Ulcer Col-M | 2.1 | 0.8 |
| 112382 Ulcer Col-M | 8.7 | 8.5 |
| 112394 Match Control Ulcer Col-M | 1.5 | 4.7 |
| 112383 Ulcer Col-M | 45.7 | 54.7 |
| 112736 Match Control Ulcer Col-M | 6.1 | 6.4 |
| 112423 Psoriasis-F | 7.7 | 5.2 |
| 112427 Match Control Psoriasis-F | 0.0 | 30.6 |
| 112418 Psoriasis-M | 8.6 | 8.7 |
| 112723 Match Control Psoriasis-M | 11.0 | 8.8 |
| 112419 Psoriasis-M | 10.7 | 8.1 |
| 112424 Match Control Psoriasis-M | 7.4 | 4.1 |
| 112420 Psoriasis-M | 37.4 | 36.3 |
| 112425 Match Control Psoriasis-M | 11.7 | 6.2 |
| 104689 (MF) OA Bone-Backus | 100.0 | 100.0 |
| 104690 (MF) Adj “Normal” | 62.0 | 65.5 |
| Bone-Backus | | |
| 104691 (MF) OA Synovium-Backus | 73.7 | 74.7 |
| 104692 (BA) OA Cartilage-Backus | 15.8 | 15.0 |
| 104694 (BA) OA Bone-Backus | 69.3 | 79.0 |
| 104695 (BA) Adj “Normal” | 68.3 | 44.1 |
| Bone-Backus | | |
| 104696 (BA) OA Synovium-Backus | 29.5 | 27.9 |
| 104700 (SS) OA Bone-Backus | 55.1 | 43.2 |
| 104701 (SS) Adj “Normal” | 72.2 | 95.3 |
| Bone-Backus | | |
| 104702 (SS) OA Synovium-Backus | 36.3 | 37.9 |
| 117093 OA Cartilage Rep7 | 4.9 | 11.3 |
| 112672 OA Bone5 | 25.3 | 25.0 |
| 112673 OA Synovium5 | 8.4 | 12.6 |
| 112674 OA Synovial Fluid | 18.8 | 16.2 |
| cells5 | | |
| 117100 OA Cartilage Rep14 | 8.0 | 10.5 |
| 112756 OA Bone9 | 3.6 | 11.2 |
| 112757 OA Synovium9 | 6.0 | 5.4 |
| 112758 OA Synovial Fluid | 9.9 | 9.4 |
| Cells9 | | |
| 117125 RA Cartilage Rep2 | 5.3 | 9.3 |
| 113492 Bone2 RA | 4.0 | 4.1 |
| 113493 Synovium2 RA | 1.0 | 1.7 |
| 113494 Syn Fluid Cells RA | 2.6 | 5.6 |
| 113499 Cartilage4 RA | 4.7 | 5.2 |
| 113500 Bone4 RA | 4.0 | 4.6 |
| 113501 Synovium4 RA | 3.6 | 3.1 |
| 113502 Syn Fluid Cells4 RA | 2.3 | 1.9 |
| 113495 Cartilage3 RA | 3.3 | 5.4 |
| 113496 Bone3 RA | 4.6 | 6.4 |
| 113497 Synovium3 RA | 3.1 | 1.6 |
| 113498 Syn Fluid Cells3 RA | 6.9 | 6.0 |
| 117106 Normal Cartilage Rep20 | 13.7 | 13.0 |
| 113663 Bone3 Normal | 0.0 | 0.0 |
| 113664 Synovium3 Normal | 0.0 | 0.0 |
| 113665 Syn Fluid Cells3 | 0.0 | 0.1 |
| Normal | | |
| 117107 Normal Cartilage Rep22 | 2.3 | 0.3 |
| 113667 Bone4 Normal | 8.6 | 4.7 |
| 113668 Synovium4 Normal | 3.0 | 6.4 |
| 113669 Syn Fluid Cells4 Normal | 12.7 | 11.0 |

TABLE AOF
CNS_neurodegeneration_v1.0

| | Rel. Exp. (%) |
|---|---|
| | Ag813, Run |
| Tissue Name | 209990454 |
| AD 1 Hippo | 43.5 |
| AD 2 Hippo | 50.0 |
| AD 3 Hippo | 35.4 |
| AD 4 Hippo | 27.7 |
| AD 5 hippo | 100.0 |
| AD 6 Hippo | 42.9 |
| Control 2 Hippo | 29.3 |
| Control 4 Hippo | 39.2 |
| Control (Path) 3 Hippo | 27.4 |
| AD 1 Temporal Ctx | 79.6 |
| AD 2 Temporal Ctx | 55.5 |
| AD 3 Temporal Ctx | 40.1 |
| AD 4 Temporal Ctx | 52.1 |
| AD 5 Inf Temporal Ctx | 84.7 |
| AD 5 Sup Temporal Ctx | 79.0 |
| AD 6 Inf Temporal Ctx | 51.8 |
| AD 6 Sup Temporal Ctx | 93.3 |
| Control 1 Temporal Ctx | 22.5 |
| Control 2 Temporal Ctx | 54.0 |
| Control 3 Temporal Ctx | 50.0 |
| Control 4 Temporal Ctx | 41.5 |
| Control (Path) 1 Temporal Ctx | 97.9 |
| Control (Path) 2 Temporal Ctx | 69.3 |
| Control (Path) 3 Temporal Ctx | 32.1 |
| Control (Path) 4 Temporal Ctx | 57.0 |
| AD 1 Occipital Ctx | 60.7 |
| AD 2 Occipital Ctx (Missing) | 0.0 |
| AD 3 Occipital Ctx | 33.4 |
| AD 4 Occipital Ctx | 48.6 |
| AD 5 Occipital Ctx | 57.8 |
| AD 6 Occipital Ctx | 43.5 |
| Control 1 Occipital Ctx | 14.4 |
| Control 2 Occipital Ctx | 73.2 |
| Control 3 Occipital Ctx | 85.3 |
| Control 4 Occipital Ctx | 28.9 |
| Control (Path) 1 Occipital Ctx | 69.7 |
| Control (Path) 2 Occipital Ctx | 49.3 |
| Control (Path) 3 Occipital Ctx | 23.3 |
| Control (Path) 4 Occipital Ctx | 57.0 |
| Control 1 Parietal Ctx | 22.2 |
| Control 2 Parietal Ctx | 84.1 |
| Control 3 Parietal Ctx | 27.9 |
| Control (Path) 1 Parietal Ctx | 68.8 |
| Control (Path) 2 Parietal Ctx | 54.3 |
| Control (Path) 3 Parietal Ctx | 26.4 |
| Control (Path) 4 Parietal Ctx | 78.5 |

TABLE AOG
General_screening_panel_v1.5

| | Rel. Exp. (%) |
|---|---|
| | Ag813, Run |
| Tissue Name | 247945092 |
| Adipose | 15.2 |
| Melanoma* Hs688(A).T | 26.2 |
| Melanoma* Hs688(B).T | 42.6 |
| Melanoma* M14 | 0.0 |
| Melanoma* LOXIMVI | 4.7 |
| Melanoma* SK-MEL-5 | 0.0 |
| Squamous cell carcinoma SCC-4 | 0.0 |
| Testis Pool | 9.6 |
| Prostate ca.* (bone met) PC-3 | 0.0 |
| Prostate Pool | 4.6 |
| Placenta | 36.6 |
| Uterus Pool | 5.4 |
| Ovarian ca. OVCAR-3 | 0.0 |
| Ovarian ca. SK-OV-3 | 1.9 |
| Ovarian ca. OVCAR-4 | 1.2 |
| Ovarian ca. OVCAR-5 | 14.3 |
| Ovarian ca. IGROV-1 | 9.7 |
| Ovarian ca. OVCAR-8 | 24.1 |
| Ovary | 20.6 |
| Breast ca. MCF-7 | 0.0 |
| Breast ca. MDA-MB-231 | 0.0 |
| Breast ca. BT 549 | 15.7 |
| Breast ca. T47D | 1.0 |
| Breast ca. MDA-N | 0.0 |
| Breast Pool | 30.1 |
| Trachea | 6.0 |
| Lung | 4.9 |
| Fetal Lung | 59.5 |
| Lung ca. NCI-N417 | 0.0 |
| Lung ca. LX-1 | 0.0 |
| Lung ca. NCI-H146 | 0.0 |
| Lung ca. SHP-77 | 1.5 |
| Lung ca. A549 | 75.3 |
| Lung ca. NCI-H526 | 0.0 |
| Lung ca NCI-H23 | 30.6 |
| Lung ca. NCI-H460 | 0.3 |
| Lung ca. HOP-62 | 19.9 |
| Lung ca. NCI-H522 | 17.7 |
| Liver | 0.4 |
| Fetal Liver | 6.3 |
| Liver ca. HepG2 | 0.0 |
| Kidney Pool | 36.6 |
| Fetal Kidney | 36.6 |
| Renal ca. 786-0 | 0.0 |
| Renal ca. A498 | 55.5 |
| Renal ca. ACHN | 0.0 |
| Renal ca. UO-31 | 7.0 |
| Renal ca. TK-10 | 50.3 |
| Bladder | 88.3 |
| Gastric ca. (liver met.) NCI-N87 | 0.0 |
| Gastric ca. KATO III | 0.0 |
| Colon ca. SW-948 | 0.0 |
| Colon ca. SW480 | 0.3 |
| Colon ca.* (SW480 met) SW620 | 0.0 |
| Colon ca. HT29 | 0.0 |
| Colon ca. HCT-116 | 3.2 |
| Colon ca. CaCo-2 | 0.4 |
| Colon cancer tissue | 30.1 |
| Colon ca. SW1116 | 0.0 |
| Colon ca. Colo-205 | 0.0 |
| Colon ca. SW-48 | 0.0 |
| Colon Pool | 29.7 |
| Small Intestine Pool | 9.5 |
| Stomach Pool | 16.3 |
| Bone Marrow Pool | 7.9 |
| Fetal Heart | 11.8 |
| Heart Pool | 10.9 |
| Lymph Node Pool | 31.4 |
| Fetal Skeletal Muscle | 15.7 |
| Skeletal Muscle Pool | 4.1 |
| Spleen Pool | 12.3 |
| Thymus Pool | 37.1 |
| CNS cancer (glio/astro) U87-MG | 0.0 |
| CNS cancer (glio/astro) U-118-MG | 0.7 |
| CNS cancer (neuro; met) SK-N-AS | 0.6 |
| CNS cancer (astro) SF-539 | 15.0 |
| CNS cancer (astro) SNB-75 | 100.0 |
| CNS cancer (glio) SNB-19 | 10.7 |
| CNS cancer (glio) SF-295 | 14.8 |
| Brain (Amygdala) Pool | 13.7 |
| Brain (cerebellum) | 8.5 |
| Brain (fetal) | 95.9 |
| Brain (Hippocampus) Pool | 12.9 |
| Cerebral Cortex Pool | 20.0 |
| Brain (Substantia nigra) Pool | 10.5 |
| Brain (Thalamus) Pool | 22.2 |
| Brain (whole) | 12.0 |
| Spinal Cord Pool | 21.0 |
| Adrenal Gland | 19.2 |
| Pituitary gland Pool | 1.6 |
| Salivary Gland | 0.4 |
| Thyroid (female) | 4.0 |
| Pancreatic ca. CAPAN2 | 1.1 |
| Pancreas Pool | 45.7 |

TABLE AOH
Panel 1.2

| | Rel. | Rel. |
|---|---|---|
| | Exp. (%) | Exp. (%) |
| | Ag813, | Ag813, |
| | Run | Run |
| Tissue Name | 118348494 | 126741639 |
| Endothelial cells | 0.0 | 0.5 |
| Heart (Fetal) | 0.0 | 8.5 |
| Pancreas | 9.9 | 27.7 |
| Pancreatic ca. CAPAN 2 | 0.0 | 0.1 |
| Adrenal Gland | 15.3 | 79.0 |
| Thyroid | 0.2 | 13.8 |
| Salivary gland | 1.8 | 15.9 |
| Pituitary gland | 9.0 | 16.7 |
| Brain (fetal) | 100.0 | 100.0 |
| Brain (whole) | 15.2 | 33.7 |
| Brain (amygdala) | 11.4 | 22.7 |
| Brain (cerebellum) | 0.3 | 8.1 |
| Brain (hippocampus) | 23.2 | 49.7 |
| Brain (thalamus) | 3.1 | 10.4 |
| Cerebral Cortex | 14.9 | 59.9 |
| Spinal cord | 6.2 | 29.7 |
| glio/astro U87-MG | 0.0 | 0.0 |
| glio/astro U-118-MG | 0.0 | 0.1 |
| astrocytoma SW1783 | 0.0 | 0.0 |
| neuro*; met SK-N-AS | 0.0 | 0.3 |
| astrocytoma SF-539 | 0.3 | 7.7 |
| astrocytoma SNB-75 | 0.1 | 4.2 |
| glioma SNB-19 | 0.0 | 8.8 |
| glioma U251 | 0.0 | 7.6 |
| glioma SF-295 | 0.0 | 1.8 |
| Heart | 7.6 | 36.9 |
| Skeletal Muscle | 0.7 | 22.5 |
| Bone marrow | 0.5 | 3.8 |
| Thymus | 6.8 | 17.2 |
| Spleen | 1.1 | 9.8 |
| Lymph node | 9.1 | 32.3 |
| Colorectal Tissue | 0.0 | 1.4 |
| Stomach | 1.3 | 27.2 |
| Small intestine | 7.4 | 28.9 |
| Colon ca. SW480 | 0.0 | 0.0 |
| Colon ca. * SW620 (SW480 met) | 0.0 | 0.0 |
| Colon ca. HT29 | 0.0 | 0.0 |
| Colon ca. HCT-116 | 0.0 | 1.1 |
| Colon ca. CaCo-2 | 0.0 | 0.3 |
| Colon ca. Tissue (ODO3866) | 0.1 | 4.6 |
| Colon ca. HCC-2998 | 0.0 | 0.0 |
| Gastric ca. * (liver met) NCI-N87 | 0.0 | 0.0 |
| Bladder | 15.8 | 92.0 |
| Trachea | 0.7 | 9.9 |
| Kidney | 1.7 | 16.7 |
| Kidney (fetal) | 11.7 | 67.4 |
| Renal ca. 786-0 | 0.0 | 0.0 |
| Renal ca. A498 | 0.0 | 7.5 |
| Renal ca. RXF 393 | 0.0 | 4.7 |
| Renal ca. ACHN | 0.0 | 0.0 |
| Renal ca. UO-31 | 0.0 | 2.3 |
| Renal ca. TK-10 | 5.8 | 18.9 |
| Liver | 5.0 | 27.9 |
| Liver (fetal) | 1.5 | 12.3 |
| Liver ca. (hepatoblast) HepG2 | 0.0 | 0.0 |
| Lung | 2.3 | 22.2 |
| Lung (fetal) | 9.9 | 46.3 |
| Lung ca. (small cell) LX-1 | 0.0 | 0.0 |
| Lung ca. (small cell) | 0.0 | 0.0 |
| NCI-H69 | | |
| Lung ca. (s. cell var.) | 0.0 | 0.5 |
| SHP-77 | | |
| Lung ca. (large cell) NCI-H460 | 0.0 | 1.1 |
| Lung ca. (non-sm. cell) A549 | 29.9 | 55.5 |
| Lung ca. (non-s. cell) NCI-H23 | 1.9 | 3.7 |
| Lung ca. (non-s. cell) HOP-62 | 4.1 | 24.0 |
| Lung ca. (non-s. cl) NCI-H522 | 4.4 | 19.9 |
| Lung ca. (squam.) SW 900 | 0.7 | 8.3 |
| Lung ca. (squam.) NCI-H596 | 0.0 | 0.0 |
| Mammary gland | 3.4 | 31.2 |
| Breast ca. * (pl. ef) MCF-7 | 0.0 | 0.0 |
| Breast ca. * (pl. ef) MDA-MB-231 | 0.0 | 0.0 |
| Breast ca. * (pl. ef) T47D | 0.2 | 6.0 |
| Breast ca. BT-549 | 0.0 | 3.5 |
| Breast ca. MDA-N | 0.0 | 0.0 |
| Ovary | 2.7 | 15.7 |
| Ovarian ca. OVCAR-3 | 0.0 | 0.2 |
| Ovarian ca. OVCAR-4 | 0.0 | 1.0 |
| Ovarian ca. OVCAR-5 | 0.2 | 4.0 |
| Ovarian ca. OVCAR-8 | 2.6 | 28.7 |
| Ovarian ca. IGROV-1 | 0.0 | 2.1 |
| Ovarian ca. (ascites) SK-OV-3 | 0.0 | 0.3 |
| Uterus | 8.1 | 36.1 |
| Placenta | 3.5 | 14.8 |
| Prostate | 0.1 | 16.8 |
| Prostate ca. * (bone met) PC-3 | 0.0 | 0.2 |
| Testis | 2.7 | 7.2 |
| Melanoma Hs688(A).T | 0.9 | 5.4 |
| Melanoma* (met) Hs688(B).T | 0.8 | 8.1 |
| Melanoma UACC-62 | 0.0 | 0.5 |
| Melanoma M14 | 0.0 | 0.0 |
| Melanoma LOX IMVI | 0.0 | 0.5 |
| Melanoma* (met) SK-MEL-5 | 0.0 | 0.0 |

TABLE AOI
Panel 4.1D

| | Rel. Exp. (%) |
|---|---|
| | Ag813, Run |
| Tissue Name | 237369996 |
| Secondary Th1 act | 4.8 |
| Secondary Th2 act | 11.7 |
| Secondary Tr1 act | 5.3 |
| Secondary Th1 rest | 4.9 |
| Secondary Th2 rest | 2.1 |
| Secondary Tr1 rest | 0.0 |
| Primary Th1 act | 0.0 |
| Primary Th2 act | 61.1 |
| Primary Tr1 act | 25.9 |
| Primary Th1 rest | 14.7 |
| Primary Th2 rest | 15.8 |
| Primary Tr1 rest | 4.1 |
| CD45RA CD4 lymphocyte act | 5.4 |
| CD45RO CD4 lymphocyte act | 22.2 |
| CD8 lymphocyte act | 0.9 |
| Secondary CD8 lymphocyte rest | 6.0 |
| Secondary CD8 lymphocyte act | 0.0 |
| CD4 lymphocyte none | 6.0 |
| 2ry Th1/Th2/Tr1_anti-CD95 CH11 | 7.1 |
| LAK cells rest | 11.3 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 10.7 |
| NK Cells IL-2 rest | 100.0 |
| Two Way MLR 3 day | 13.9 |
| Two Way MLR 5 day | 0.0 |
| Two Way MLR 7 day | 0.0 |
| PBMC rest | 2.9 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 2.4 |
| B lymphocytes CD40L and IL-4 | 2.9 |
| EOL-1 dbcAMP | 80.1 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 14.2 |
| HUVEC IL-1beta | 11.6 |
| HUVEC IFN gamma | 6.9 |
| HUVEC TNF alpha + IFN gamma | 2.7 |
| HUVEC TNF alpha + IL4 | 1.3 |
| HUVEC IL-11 | 20.0 |
| Lung Microvascular EC none | 95.9 |
| Lung Microvascular EC TNFalpha + IL-1beta | 39.0 |
| Microvascular Dermal EC none | 2.8 |
| Microsvasular Dermal EC TNFalpha + IL-1beta | 6.7 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 33.4 |
| Coronery artery SMC TNFalpha + IL-1beta | 31.4 |
| Astrocytes rest | 20.4 |
| Astrocytes TNFalpha + IL-1beta | 6.9 |
| KU-812 (Basophil) rest | 0.0 |
| KU-812 (Basophil) PMA/ionomycin | 11.0 |
| CCD1106 (Keratinocytes) none | 0.0 |
| CCD1106 (Keratinocytes) TNFalpha + IL-1beta | 0.0 |
| Liver cirrhosis | 32.5 |
| NCI-H292 none | 0.0 |
| NCI-H292 IL-4 | 0.0 |
| NCI-H292 IL-9 | 0.0 |
| NCI-H292 IL-13 | 5.4 |
| NCI-H292 IFN gamma | 0.0 |
| HPAEC none | 5.3 |
| HPAEC TNF alpha + IL-1 beta | 4.9 |
| Lung fibroblast none | 10.7 |
| Lung fibroblast TNF alpha + IL-1 beta | 9.4 |
| Lung fibroblast IL-4 | 7.8 |
| Lung fibroblast IL-9 | 6.8 |
| Lung fibroblast IL-13 | 0.0 |
| Lung fibroblast IFN gamma | 16.5 |
| Dermal fibroblast CCD1070 rest | 12.2 |
| Dermal fibroblast CCD1070 TNF alpha | 25.5 |
| Dermal fibroblast CCD1070 IL-1 beta | 20.3 |
| Dermal fibroblast IFN gamma | 9.9 |
| Dermal fibroblast IL-4 | 57.8 |
| Dermal Fibroblasts rest | 13.0 |
| Neutrophils TNFa + LPS | 0.0 |
| Neutrophils rest | 2.5 |
| Colon | 0.0 |
| Lung | 0.0 |
| Thymus | 4.8 |
| Kidney | 21.9 |

TABLE AOJ
Panel 5 Islet

| | Rel. Exp. (%) |
|---|---|
| | Ag813, Run |
| Tissue Name | 254387841 |
| 97457_Patient-02go_adipose | 45.7 |
| 97476_Patient-07sk_skeletal muscle | 11.7 |
| 97477_Patient-07ut_uterus | 33.2 |
| 97478_Patient-07pl_placenta | 11.7 |
| 99167_Bayer Patient 1 | 14.4 |
| 97482_Patient-08ut_uterus | 45.7 |
| 97483_Patient-08pl_placenta | 7.0 |
| 97486_Patient-09sk_skeletal muscle | 0.0 |
| 97487_Patient-09ut_uterus | 16.3 |
| 97488_Patient-09pl_placenta | 13.8 |
| 97492_Patient-10ut_uterus | 24.3 |
| 97493_Patient-10pl_placenta | 5.1 |
| 97495_Patient-11go_adipose | 9.7 |
| 97496_Patient-11sk_skeletal muscle | 15.0 |
| 97497_Patient-11ut_uterus | 43.2 |
| 97498_Patient-11pl_placenta | 7.9 |
| 97500_Patient-12go_adipose | 36.3 |
| 97501_Patient-12sk_skeletal muscle | 33.2 |
| 97502_Patient-12ut_uterus | 55.1 |
| 97503_Patient-12pl_placenta | 0.0 |
| 94721_Donor 2 U - A_Mesenchymal Stem Cells | 66.0 |
| 94722_Donor 2 U - B_Mesenchymal Stem Cells | 32.1 |
| 94723_Donor 2 U - C_Mesenchymal Stem Cells | 62.0 |
| 94709_Donor 2 AM - A_adipose | 49.3 |
| 94710_Donor 2 AM - B_adipose | 15.8 |
| 94711_Donor 2 AM - C_adipose | 8.4 |
| 94712_Donor 2 AD - A_adipose | 52.9 |
| 94713_Donor 2 AD - B_adipose | 36.3 |
| 94714_Donor 2 AD - C_adipose | 35.6 |
| 94742_Donor 3 U - A_Mesenchymal Stem Cells | 27.4 |
| 94743_Donor 3 U - B_Mesenchymal Stem Cells | 33.9 |
| 94730_Donor 3 AM - A_adipose | 17.2 |
| 94731_Donor 3 AM - B_adipose | 21.2 |
| 94732_Donor 3 AM - C_adipose | 4.9 |
| 94733_Donor 3 AD - A_adipose | 100.0 |
| 94734_Donor 3 AD - B_adipose | 40.3 |
| 94735_Donor 3 AD - C_adipose | 69.7 |
| 77138_Liver_HepG2untreated | 0.0 |
| 73556_Heart_Cardiac stromal cells (primary) | 7.9 |
| 81735_Small Intestine | 54.3 |
| 72409_Kidney_Proximal Convoluted Tubule | 0.0 |
| 82685_Small intestine_Duodenum | 0.0 |
| 90650_Adrenal_Adrenocortical adenoma | 26.2 |
| 72410_Kidney_HRCE | 0.0 |
| 72411_Kidney_HRE | 0.0 |
| 73139_Uterus_Uterine smooth muscle cells | 6.3 |

TABLE AOK
Panel CNS_1

| | Rel. Exp. (%) |
|---|---|
| | Ag813, Run |
| Tissue Name | 171629144 |
| BA4 Control | 3.0 |
| BA4 Control2 | 22.7 |
| BA4 Alzheimer's2 | 6.6 |
| BA4 Parkinson's | 36.6 |
| BA4 Parkinson's2 | 49.3 |
| BA4 Huntington's | 8.4 |
| BA4 Huntington's2 | 12.0 |
| BA4 PSP | 5.9 |
| BA4 PSP2 | 6.7 |
| BA4 Depression | 11.0 |
| BA4 Depression2 | 19.6 |
| BA7 Control | 19.9 |
| BA7 Control2 | 18.9 |
| BA7 Alzheimer's2 | 12.5 |
| BA7 Parkinson's | 33.9 |
| BA7 Parkinson's2 | 31.4 |
| BA7 Huntington's | 37.4 |
| BA7 Huntington's2 | 100.0 |
| BA7 PSP | 21.9 |
| BA7 PSP2 | 3.8 |
| BA7 Depression | 6.2 |
| BA9 Control | 22.2 |
| BA9 Control2 | 23.5 |
| BA9 Alzheimer's | 0.0 |
| BA9 Alzheimer's2 | 19.8 |
| BA9 Parkinson's | 42.6 |
| BA9 Parkinson's2 | 21.6 |
| BA9 Huntington's | 17.7 |
| BA9 Huntington's2 | 52.5 |
| BA9 PSP | 6.8 |
| BA9 PSP2 | 2.7 |
| BA9 Depression | 6.7 |
| BA9 Depression2 | 10.4 |
| BA17 Control | 43.5 |
| BA17 Control2 | 24.1 |
| BA17 Alzheimer's2 | 21.2 |
| BA17 Parkinson's | 33.4 |
| BA17 Parkinson's2 | 39.0 |
| BA17 Huntington's | 24.0 |
| BA17 Huntington's2 | 37.9 |
| BA17 Depression | 31.9 |
| BA17 Depression2 | 45.7 |
| BA17 PSP | 3.9 |
| BA17 PSP2 | 4.8 |
| Sub Nigra Control | 14.8 |
| Sub Nigra Control2 | 16.5 |
| Sub Nigra Alzheimer's2 | 6.1 |
| Sub Nigra Parkinson's2 | 23.8 |
| Sub Nigra Huntington's | 14.6 |
| Sub Nigra Huntington's2 | 32.8 |
| Sub Nigra PSP2 | 0.0 |
| Sub Nigra Depression | 2.5 |
| Sub Nigra Depression2 | 7.6 |
| Glob Palladus Control | 2.5 |
| Glob Palladus Control2 | 0.7 |
| Glob Palladus Alzheimer's | 4.6 |
| Glob Palladus Alzheimer's2 | 3.2 |
| Glob Palladus Parkinson's | 41.8 |
| Glob Palladus Parkinson's2 | 11.4 |
| Glob Palladus PSP | 5.0 |
| Glob Palladus PSP2 | 0.0 |
| Glob Palladus Depression | 1.7 |
| Temp Pole Control | 2.9 |
| Temp Pole Control2 | 4.8 |
| Temp Pole Alzheimer's | 2.5 |
| Temp Pole Alzheimer's2 | 12.4 |
| Temp Pole Parkinson's | 28.9 |
| Temp Pole Parkinson's2 | 13.1 |
| Temp Pole Huntington's | 28.1 |
| Temp Pole PSP | 6.9 |
| Temp Pole PSP2 | 1.9 |
| Temp Pole Depression2 | 18.8 |
| Cing Gyr Control | 39.0 |
| Cing Gyr Control2 | 16.4 |
| Cing Gyr Alzheimer's | 4.8 |
| Cing Gyr Alzheimer's2 | 7.2 |
| Cing Gyr Parkinson's | 22.4 |
| Cing Gyr Parkinson's2 | 9.2 |
| Cing Gyr Huntington's | 24.3 |
| Cing Gyr Huntington's2 | 33.9 |
| Cing Gyr PSP | 5.2 |
| Cing Gyr PSP2 | 0.0 |
| Cing Gyr Depression | 9.5 |
| Cing Gyr Depression2 | 0.0 |

AI_comprehensive panel_v1.0 Summary: Ag813 Two experiments with same probe-primer sets are in excellent agreement. Highest expression of this gene is detected in orthoarthritis bone (CTs=29-30.6). In addition significant expression of this gene is detected in samples derived from orthoarthritis bone, cartilage, synovium and synovial fluid samples, from normal lung, COPD lung, emphysema, atopic asthma, asthma, allergy, Crohn's disease (normal matched control and diseased), ulcerative colitis(normal matched control and diseased), and psoriasis (normal matched control and diseased). Interestingly, expression of this gene in normal and rheumatoid arthritis bone, synovium and synovial fluid is very low or undectectable. Therefore, therapeutic modulation of this gene product may ameliorate symptoms/conditions associated with autoimmune and inflammatory disorders including psoriasis, allergy, asthma, inflammatory bowel disease, and osteoarthritis.

CNS_neurodegeneration_v1.0 Summary: Ag813 This panel confirms the expression of this gene at low levels in the brains of an independent group of individuals. However, no differential expression of this gene was detected between Alzheimer's diseased postmortem brains and those of non-demented controls in this experiment. Please see Panel 1.5 for a discussion of the potential utility of this gene in treatment of central nervous system disorders.

General_screening_panel_v1.5 Summary: Ag813 Highest expression of this gene is detected in fetal brain and brain cancer SNB-75 cell line (CTs=31). In addition, moderate expression of this gene is seen all regions of the central nervous system examined, including amygdala, hippocampus, substantia nigra, thalamus, cerebellum, cerebral cortex, and spinal cord. This gene codes for a variant of ADAMTS- 10, a member of Matrix metalloproteinases (MMPs). MMPs are a gene family of neutral proteases that are important in normal development, wound healing, and a wide variety of pathological processes, including the spread of metastatic cancer cells, arthritic destruction of joints, atherosclerosis, and neuroinflammation. In the central nervous system (CNS), MMPs have been shown to degrade components of the basal lamina, leading to disruption of the blood-brain barrier (BBB), and to contribute to the neuroinflammatory response in many neurological diseases (Rosenberg Ga., 2002, Glia 39(3):279-91, PMID: 12203394). Therefore, therapeutic modulation of this gene product may be useful in the treatment of neurological disorders such as Alzheimer's disease, Parkinson's disease, epilepsy, multiple sclerosis, schizophrenia, depression, allergic encephalomyelitis (EAE), allergic neuritis (EAN), and cerebral ischemia.

Moderate to low expression of this gene is also detected in tissues with metabolic/endocrine function including pancreas, adipose, adrenal gland, skeletal muscle, heart, fetal liver and the gastrointestinal tract. Therefore, therapeutic modulation of the activity of this gene may prove useful in the treatment of endocrine/metabolically related diseases, such as obesity and diabetes.

In addition, this gene is expressed at moderate to low levels in number of cancer cell lines derived from melanoma, ovarian, breast, lung, renal, colon and brain cancers. Therefore, therapeutic modulation of this gene through the use of protein therapeutics, antibodies or small molecule drug may be useful in the treatment of these cancer.

Using Curagen PathCalling technology, the ADAMTS-10 protein encoded by this gene was shown to interact with amphiregulin (AREG). AREG is shown to inhibit growth of certain human tumor cells and stimulates proliferation of human fibroblasts and other normal and tumor cells (Shoyab et al., 1988, Proc. Nat. Acad. Sci. 85: 6528-6532. PubMed ID: 3413110). Recently, AREG has been implicated in the regulation of neural stem cell proliferation and neurogenesis in the adult brain.

Panel 1.2 Summary: Ag813 Highest expression of this gene is detected in fetal brain (CT=27.5). In addition, moderate expression of this gene is all regions of the central nervous system examined, including amygdala, hippocampus, substantia nigra, thalamus, cerebellum, cerebral cortex, and spinal cord. Moderate to low expression of this gene is also detected in tissues with metabolic/endocrine function and number of cancer cell lines derived from melanoma, ovarian, lung, renal, colon and brain cancers. Please see panel 1.5 for further discussion on the utility of this gene.

Panel 4.1D Summary: Ag813 Highest expression of this gene is detected in IL-2 treated resting NK cells (CT=32.8). Moderate to low levels of expression of this gene is also detected in activated primary polarized T cells, eosinophils, lung microvascular endothelial cells, coronery artery SMC, liver cirrhosis and activated dermal fibroblasts. Therefore, therapeutic modulation of this gene or the protein encoded by this gene may be useful in the treatment of autoimmune and inflammatory diseases including asthma, allergies, inflammatory bowel disease, lupus erythematosus, psoriasis, rheumatoid arthritis, and osteoarthritis.

Results from one experiment (Run 247683477) with this gene are not included. The amp plot indicates that there were experimental difficulties with this run.

Panel 5 Islet Summary: Ag813 Highest expression of this gene is detected in differentiated adipose (CT=33.5). Low expression of this gene is seen mainly in adipose and small intestine. Therefore, therapeutic modulation of this gene or its protein product may be useful in the treatment of obesity and diabetes, including Type II diabetes.

Panel CNS_{—}1 Summary: Ag813 This panel confirms the expression of this gene at low levels in the brains of an independent group of individuals. Please see Panel 1.5 for a discussion of the potential utility of this gene in treatment of central nervous system disorders.

AP. CG56155-02: Plasma Kallikrein Precursor.

Expression of gene CG56155-02 was assessed using the primer-probe set Ag1688, described in Table APA. Results of the RTQ-PCR runs are shown in Tables APB, APC, APD, APE, APF, APG and APH.

TABLE APA
Probe Name Ag1688

| | | | Start | SEQ ID |
|---|---|---|---|---|
| Primers | Sequences | Length | Position | No |
| Forward | 5′-tcagaagggaatcatgatatcg-3′ | 22 | 577 | 606 |
| Probe | TET-5′-ccttgataaaactccaggctcctttga-3′-TAMRA | 27 | 550 | 607 |
| Reverse | 5′-tttggaaggtaggcatattgg-3′ | 21 | 509 | 608 |

TABLE APB
AI_comprehensive panel_v1.0

| | Rel. Exp. (%) |
|---|---|
| | Ag1688, Run |
| Tissue Name | 248429492 |
| 110967 COPD-F | 53.6 |
| 110980 COPD-F | 14.2 |
| 110968 COPD-M | 48.3 |
| 110977 COPD-M | 53.6 |
| 110989 Emphysema-F | 61.6 |
| 110992 Emphysema-F | 21.6 |
| 110993 Emphysema-F | 23.8 |
| 110994 Emphysema-F | 20.7 |
| 110995 Emphysema-F | 55.1 |
| 110996 Emphysema-F | 17.8 |
| 110997 Asthma-M | 25.2 |
| 111001 Asthma-F | 23.0 |
| 111002 Asthma-F | 22.1 |
| 111003 Atopic Asthma-F | 15.5 |
| 111004 Atopic Asthma-F | 19.9 |
| 111005 Atopic Asthma-F | 23.8 |
| 111006 Atopic Asthma-F | 6.0 |
| 111417 Allergy-M | 4.6 |
| 112347 Allergy-M | 0.0 |
| 112349 Normal Lung-F | 0.0 |
| 112357 Normal Lung-F | 38.7 |
| 112354 Normal Lung-M | 24.0 |
| 112374 Crohns-F | 10.4 |
| 112389 Match Control Crohns-F | 7.4 |
| 112375 Crohns-F | 4.6 |
| 112732 Match Control Crohns-F | 25.0 |
| 112725 Crohns-M | 11.3 |
| 112387 Match Control Crohns-M | 1.0 |
| 112378 Crohns-M | 0.0 |
| 112390 Match Control Crohns-M | 44.1 |
| 112726 Crohns-M | 19.5 |
| 112731 Match Control Crohns-M | 58.2 |
| 112380 Ulcer Col-F | 3.2 |
| 112734 Match Control Ulcer Col-F | 56.6 |
| 112384 Ulcer Col-F | 10.1 |
| 112737 Match Control Ulcer Col-F | 21.6 |
| 112386 Ulcer Col-F | 0.0 |
| 112738 Match Control Ulcer Col-F | 9.3 |
| 112381 Ulcer Col-M | 0.0 |
| 112735 Match Control Ulcer Col-M | 41.8 |
| 112382 Ulcer Col-M | 3.8 |
| 112394 Match Control Ulcer Col-M | 5.2 |
| 112383 Ulcer Col-M | 31.6 |
| 112736 Match Control Ulcer Col-M | 12.9 |
| 112423 Psoriasis-F | 9.2 |
| 112427 Match Control Psoriasis-F | 77.4 |
| 112418 Psoriasis-M | 12.7 |
| 112723 Match Control Psoriasis-M | 0.0 |
| 112419 Psoriasis-M | 100.0 |
| 112424 Match Control Psoriasis-M | 35.6 |
| 112420 Psoriasis-M | 87.7 |
| 112425 Match Control Psoriasis-M | 29.1 |
| 104689 (MF) OA Bone-Backus | 50.0 |
| 104690 (MF) Adj “Normal” Bone-Backus | 34.9 |
| 104691 (MF) OA Synovium-Backus | 25.5 |
| 104692 (BA) OA Cartilage-Backus | 37.6 |
| 104694 (BA) OA Bone-Backus | 8.4 |
| 104695 (BA) Adj “Normal” Bone-Backus | 34.4 |
| 104696 (BA) OA Synovium-Backus | 6.9 |
| 104700 (SS) OA Bone-Backus | 22.8 |
| 104701 (SS) Adj “Normal” Bone-Backus | 42.3 |
| 104702 (SS) OA Synovium-Backus | 29.5 |
| 117093 OA Cartilage Rep7 | 10.6 |
| 112672 OA Bone5 | 94.0 |
| 112673 OA Synovium5 | 43.2 |
| 112674 OA Synovial Fluid cells5 | 58.6 |
| 117100 OA Cartilage Rep14 | 0.0 |
| 112756 OA Bone9 | 2.6 |
| 112757 OA Synovium9 | 8.0 |
| 112758 OA Synovial Fluid Cells9 | 22.1 |
| 117125 RA Cartilage Rep2 | 22.1 |
| 113492 Bone2 RA | 10.0 |
| 113493 Synovium2 RA | 11.0 |
| 113494 Syn Fluid Cells RA | 31.6 |
| 113499 Cartilage4 RA | 47.6 |
| 113500 Bone4 RA | 37.9 |
| 113501 Synovium4 RA | 55.5 |
| 113502 Syn Fluid Cells4 RA | 10.0 |
| 113495 Cartilage3 RA | 20.7 |
| 113496 Bone3 RA | 16.2 |
| 113497 Synovium3 RA | 11.5 |
| 113498 Syn Fluid Cells3 RA | 25.3 |
| 117106 Normal Cartilage Rep20 | 0.0 |
| 113663 Bone3 Normal | 0.9 |
| 113664 Synovium3 Normal | 0.0 |
| 113665 Syn Fluid Cells3 Normal | 1.1 |
| 117107 Normal Cartilage Rep22 | 2.7 |
| 113667 Bone4 Normal | 8.1 |
| 113668 Synovium4 Normal | 5.8 |
| 113669 Syn Fluid Cells4 Normal | 5.3 |

TABLE APC
CNS_neurodegeneration_v1.0

| | Rel. Exp. (%) |
|---|---|
| | Ag1688, Run |
| Tissue Name | 269217573 |
| AD 1 Hippo | 24.5 |
| AD 2 Hippo | 34.4 |
| AD 3 Hippo | 17.9 |
| AD 4 Hippo | 18.0 |
| AD 5 hippo | 94.6 |
| AD 6 Hippo | 34.9 |
| Control 2 Hippo | 35.4 |
| Control 4 Hippo | 50.7 |
| Control (Path) 3 Hippo | 9.3 |
| AD 1 Temporal Ctx | 31.9 |
| AD 2 Temporal Ctx | 31.4 |
| AD 3 Temporal Ctx | 20.4 |
| AD 4 Temporal Ctx | 29.5 |
| AD 5 Inf Temporal Ctx | 100.0 |
| AD 5 SupTemporal Ctx | 92.0 |
| AD 6 Inf Temporal Ctx | 43.8 |
| AD 6 Sup Temporal Ctx | 69.7 |
| Control 1 Temporal Ctx | 16.5 |
| Control 2 Temporal Ctx | 34.9 |
| Control 3 Temporal Ctx | 32.3 |
| Control 4 Temporal Ctx | 35.4 |
| Control (Path) 1 Temporal Ctx | 46.0 |
| Control (Path) 2 Temporal Ctx | 45.7 |
| Control (Path) 3 Temporal Ctx | 9.7 |
| Control (Path) 4 Temporal Ctx | 41.8 |
| AD 1 Occipital Ctx | 42.3 |
| AD 2 Occipital Ctx (Missing) | 0.0 |
| AD 3 Occipital Ctx | 7.1 |
| AD 4 Occipital Ctx | 26.4 |
| AD 5 Occipital Ctx | 9.9 |
| AD 6 Occipital Ctx | 27.2 |
| Control 1 Occipital Ctx | 6.3 |
| Control 2 Occipital Ctx | 49.7 |
| Control 3 Occipital Ctx | 39.2 |
| Control 4 Occipital Ctx | 26.6 |
| Control (Path) 1 Occipital Ctx | 47.3 |
| Control (Path) 2 Occipital Ctx | 21.3 |
| Control (Path) 3 Occipital Ctx | 3.5 |
| Control (Path) 4 Occipital Ctx | 17.8 |
| Control 1 Parietal Ctx | 19.5 |
| Control 2 Parietal Ctx | 85.3 |
| Control 3 Parietal Ctx | 15.5 |
| Control (Path) 1 Parietal Ctx | 44.4 |
| Control (Path) 2 Parietal Ctx | 52.9 |
| Control (Path) 3 Parietal Ctx | 9.7 |
| Control (Path) 4 Parietal Ctx | 52.1 |

TABLE APD
Panel 1.3D

| | Rel. Exp. (%) |
|---|---|
| | Ag1688, Run |
| Tissue Name | 147249266 |
| Liver adenocarcinoma | 0.0 |
| Pancreas | 6.7 |
| Pancreatic ca. CAPAN2 | 0.2 |
| Adrenal gland | 1.8 |
| Thyroid | 3.8 |
| Salivary gland | 1.5 |
| Pituitary gland | 6.1 |
| Brain (fetal) | 0.5 |
| Brain (whole) | 3.6 |
| Brain (amygdala) | 3.3 |
| Brain (cerebellum) | 0.4 |
| Brain (hippocampus) | 6.2 |
| Brain (substantia nigra) | 1.0 |
| Brain (thalamus) | 2.1 |
| Cerebral Cortex | 6.3 |
| Spinal cord | 3.1 |
| glio/astro U87-MG | 0.0 |
| glio/astro U-118-MG | 0.0 |
| astrocytoma SW1783 | 0.0 |
| neuro*; met SK-N-AS | 0.2 |
| astrocytoma SF-539 | 0.0 |
| astrocytoma SNB-75 | 0.1 |
| glioma SNB-19 | 0.2 |
| glioma U251 | 1.2 |
| glioma SF-295 | 0.0 |
| Heart (fetal) | 0.2 |
| Heart | 1.6 |
| Skeletal muscle (fetal) | 0.7 |
| Skeletal muscle | 1.2 |
| Bone marrow | 0.5 |
| Thymus | 3.2 |
| Spleen | 1.0 |
| Lymph node | 2.9 |
| Colorectal | 0.8 |
| Stomach | 3.3 |
| Small intestine | 6.2 |
| Colon ca. SW480 | 0.0 |
| Colon ca.* SW620(SW480 met) | 0.0 |
| Colon ca. HT29 | 0.0 |
| Colon ca. HCT-116 | 0.0 |
| Colon ca. CaCo-2 | 0.2 |
| Colon ca. tissue(ODO3866) | 0.0 |
| Colon ca. HCC-2998 | 0.2 |
| Gastric ca.* (liver met) NCI-N87 | 4.4 |
| Bladder | 3.1 |
| Trachea | 3.0 |
| Kidney | 6.8 |
| Kidney (fetal) | 9.2 |
| Renal ca. 786-0 | 0.0 |
| Renal ca. A498 | 1.7 |
| Renal ca. RXF 393 | 0.0 |
| Renal ca. ACHN | 0.0 |
| Renal ca. UO-31 | 0.0 |
| Renal ca. TK-10 | 0.0 |
| Liver | 100.0 |
| Liver (fetal) | 99.3 |
| Liver ca. (hepatoblast) HepG2 | 0.0 |
| Lung | 1.3 |
| Lung (fetal) | 1.8 |
| Lung ca. (small cell) LX-1 | 0.0 |
| Lung ca. (small cell) NCI-H69 | 0.0 |
| Lung ca. (s. cell var.) SHP-77 | 0.8 |
| Lung ca. (large cell)NCI-H460 | 0.0 |
| Lung ca. (non-sm. cell) A549 | 0.2 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.2 |
| Lung ca. (squam.) NCI-H596 | 0.0 |
| Mammary gland | 2.9 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.2 |
| Ovarian ca. OVCAR-4 | 0.0 |
| Ovarian ca. OVCAR-5 | 0.3 |
| Ovarian ca. OVCAR-8 | 0.0 |
| Ovarian ca. IGROV-1 | 0.0 |
| Ovarian ca.* (ascites) SK-OV-3 | 1.0 |
| Uterus | 1.4 |
| Placenta | 0.4 |
| Prostate | 1.0 |
| Prostate ca.* (bone met)PC-3 | 0.0 |
| Testis | 6.1 |
| Melanoma Hs688(A).T | 0.4 |
| Melanoma* (met) Hs688(B).T | 0.9 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 0.5 |

TABLE APE
Panel 2D

| | Rel. Exp. (%) |
|---|---|
| | Ag1688, Run |
| Tissue Name | 162646059 |
| Normal Colon | 1.7 |
| CC Well to Mod Diff (ODO3866) | 0.0 |
| CC Margin (ODO3866) | 0.2 |
| CC Gr.2 rectosigmoid (ODO3868) | 0.2 |
| CC Margin (ODO3868) | 0.1 |
| CC Mod Diff (ODO3920) | 0.1 |
| CC Margin (ODO3920) | 0.9 |
| CC Gr.2 ascend colon (ODO3921) | 0.1 |
| CC Margin (ODO3921) | 0.1 |
| CC from Partial Hepatectomy (ODO4309) Mets | 4.7 |
| Liver Margin (ODO4309) | 100.0 |
| Colon mets to lung (OD04451-01) | 0.1 |
| Lung Margin (OD04451-02) | 0.1 |
| Normal Prostate 6546-1 | 2.1 |
| Prostate Cancer (OD04410) | 0.6 |
| Prostate Margin (OD04410) | 0.5 |
| Prostate Cancer (OD04720-01) | 1.1 |
| Prostate Margin (OD04720-02) | 1.6 |
| Normal Lung 061010 | 2.0 |
| Lung Met to Muscle (ODO4286) | 0.0 |
| Muscle Margin (ODO4286) | 0.2 |
| Lung Malignant Cancer (OD03126) | 0.1 |
| Lung Margin (OD03126) | 0.5 |
| Lung Cancer (OD04404) | 0.1 |
| Lung Margin (OD04404) | 0.2 |
| Lung Cancer (OD04565) | 0.0 |
| Lung Margin (OD04565) | 0.1 |
| Lung Cancer (OD04237-01) | 0.1 |
| Lung Margin (OD04237-02) | 0.4 |
| Ocular Mel Met to Liver (ODO4310) | 0.1 |
| Liver Margin (ODO4310) | 77.4 |
| Melanoma Mets to Lung (OD04321) | 0.0 |
| Lung Margin (OD04321) | 0.1 |
| Normal Kidney | 12.9 |
| Kidney Ca, Nuclear grade 2 (OD04338) | 3.8 |
| Kidney Margin (OD04338) | 1.6 |
| Kidney Ca Nuclear grade 1/2 (OD04339) | 2.8 |
| Kidney Margin (OD04339) | 9.3 |
| Kidney Ca, Clear cell type (OD04340) | 1.4 |
| Kidney Margin (OD04340) | 4.1 |
| Kidney Ca, Nuclear grade 3 (OD04348) | 0.1 |
| Kidney Margin (OD04348) | 3.8 |
| Kidney Cancer (OD04622-01) | 0.2 |
| Kidney Margin (OD04622-03) | 0.7 |
| Kidney Cancer (OD04450-01) | 0.2 |
| Kidney Margin (OD04450-03) | 2.6 |
| Kidney Cancer 8120607 | 0.0 |
| Kidney Margin 8120608 | 0.7 |
| Kidney Cancer 8120613 | 0.0 |
| Kidney Margin 8120614 | 0.5 |
| Kidney Cancer 9010320 | 0.2 |
| Kidney Margin 9010321 | 1.0 |
| Normal Uterus | 0.2 |
| Uterus Cancer 064011 | 0.8 |
| Normal Thyroid | 0.9 |
| Thyroid Cancer 064010 | 0.2 |
| Thyroid Cancer A302152 | 0.5 |
| Thyroid Margin A302153 | 1.0 |
| Normal Breast | 0.3 |
| Breast Cancer (OD04566) | 0.1 |
| Breast Cancer (OD04590-01) | 0.1 |
| Breast Cancer Mets (OD04590-03) | 0.4 |
| Breast Cancer Metastasis (OD04655-05) | 0.9 |
| Breast Cancer 064006 | 0.6 |
| Breast Cancer 1024 | 1.2 |
| Breast Cancer 9100266 | 0.1 |
| Breast Margin 9100265 | 0.1 |
| Breast Cancer A209073 | 0.3 |
| Breast Margin A209073 | 0.3 |
| Normal Liver | 69.7 |
| Liver Cancer 064003 | 13.7 |
| Liver Cancer 1025 | 18.0 |
| Liver Cancer 1026 | 1.2 |
| Liver Cancer 6004-T | 22.2 |
| Liver Tissue 6004-N | 1.0 |
| Liver Cancer 6005-T | 1.9 |
| Liver Tissue 6005-N | 4.2 |
| Normal Bladder | 2.7 |
| Bladder Cancer 1023 | 0.0 |
| Bladder Cancer A302173 | 0.2 |
| Bladder Cancer (OD04718-01) | 0.1 |
| Bladder Normal Adjacent (OD04718-03) | 0.5 |
| Normal Ovary | 0.0 |
| Ovarian Cancer 064008 | 0.1 |
| Ovarian Cancer (OD04768-07) | 0.2 |
| Ovary Margin (OD04768-08) | 0.1 |
| Normal Stomach | 0.3 |
| Gastric Cancer 9060358 | 0.1 |
| Stomach Margin 9060359 | 0.0 |
| Gastric Cancer 9060395 | 0.2 |
| Stomach Margin 9060394 | 0.3 |
| Gastric Cancer 9060397 | 0.3 |
| Stomach Margin 9060396 | 0.0 |
| Gastric Cancer 064005 | 1.1 |

TABLE APF
Panel 4.1D

| | Rel. Exp. (%) |
|---|---|
| | Ag1688, Run |
| Tissue Name | 248389308 |
| Secondary Th1 act | 1.6 |
| Secondary Th2 act | 1.7 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 1.3 |
| Primary Th2 rest | 1.3 |
| Primary Tr1 rest | 1.6 |
| CD45RA CD4 lymphocyte act | 3.5 |
| CD45RO CD4 lymphocyte act | 4.2 |
| CD8 lymphocyte act | 3.2 |
| Secondary CD8 lymphocyte rest | 1.8 |
| Secondary CD8 lymphocyte act | 0.0 |
| CD4 lymphocyte none | 3.8 |
| 2ry Th1/Th2/Tr1_anti-CD95 CH11 | 0.0 |
| LAK cells rest | 0.0 |
| LAK cells IL-2 | 6.2 |
| LAK cells IL-2 + IL-12 | 0.0 |
| LAK cells IL-2 + IFN gamma | 1.7 |
| LAK cells IL-2 + IL-18 | 3.4 |
| LAK cells PMA/ionomycin | 0.0 |
| NK Cells IL-2 rest | 22.1 |
| Two Way MLR 3 day | 3.3 |
| Two Way MLR 5 day | 1.9 |
| Two Way MLR 7 day | 1.7 |
| PBMC rest | 1.5 |
| PBMC PWM | 5.1 |
| PBMC PHA-L | 0.7 |
| Ramos (B cell) none | 0.0 |
| Ramos (B cell) ionomycin | 0.0 |
| B lymphocytes PWM | 2.8 |
| B lymphocytes CD40L and IL-4 | 21.5 |
| EOL-1 dbcAMP | 0.0 |
| EOL-1 dbcAMP PMA/ionomycin | 0.0 |
| Dendritic cells none | 2.0 |
| Dendritic cells LPS | 0.0 |
| Dendritic cells anti-CD40 | 4.9 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 2.4 |
| KU-812 (Basophil) rest | 1.8 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 100.0 |
| NCI-H292 none | 0.0 |
| NCI-H292 IL-4 | 1.5 |
| NCI-H292 IL-9 | 1.9 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 2.6 |
| Lung fibroblast TNF alpha + IL-1 beta | 10.4 |
| Lung fibroblast IL-4 | 1.8 |
| Lung fibroblast IL-9 | 12.3 |
| Lung fibroblast IL-13 | 0.0 |
| Lung fibroblast IFN gamma | 3.1 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 6.8 |
| Dermal fibroblast IL-4 | 5.8 |
| Dermal Fibroblasts rest | 0.0 |
| Neutrophils TNFa + LPS | 0.0 |
| Neutrophils rest | 0.0 |
| Colon | 0.0 |
| Lung | 1.2 |
| Thymus | 0.0 |
| Kidney | 82.9 |

TABLE APG
Panel 5 Islet

| | Rel. Exp. (%) |
|---|---|
| | Ag1688, Run |
| Tissue Name | 226587524 |
| 97457_Patient-02go_adipose | 41.2 |
| 97476_Patient-07sk_skeletal muscle | 9.9 |
| 97477_Patient-07ut_uterus | 8.1 |
| 97478_Patient-07pl_placenta | 0.0 |
| 99167_Bayer Patient 1 | 84.7 |
| 97482_Patient-08ut_uterus | 2.4 |
| 97483_Patient-08pl_placenta | 0.0 |
| 97486_Patient-09sk_skeletal muscle | 8.0 |
| 97487_Patient-09ut_uterus | 9.6 |
| 97488_Patient-09pl_placenta | 0.0 |
| 97492_Patient-10ut_uterus | 0.0 |
| 97493_Patient-10pl_placenta | 0.0 |
| 97495_Patient-11go_adipose | 0.0 |
| 97496_Patient-11sk_skeletal muscle | 52.9 |
| 97497_Patient-11ut_uterus | 35.8 |
| 97498_Patient-11pl_placenta | 10.5 |
| 97500_Patient-12go_adipose | 0.0 |
| 97501_Patient-12sk_skeletal muscle | 35.4 |
| 97502_Patient-12ut_uterus | 20.7 |
| 97503_Patient-12pl_placenta | 0.0 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 11.4 |
| 94713_Donor 2 AD - B_adipose | 0.0 |
| 94714_Donor 2 AD - C_adipose | 29.1 |
| 94742_Donor 3 U - A_Mesenchymal Stem Cells | 19.2 |
| 94743_Donor 3 U - B_Mesenchymal Stem Cells | 0.0 |
| 94730_Donor 3 AM - A_adipose | 15.0 |
| 94731_Donor 3 AM - B_adipose | 37.9 |
| 94732_Donor 3 AM - C_adipose | 0.0 |
| 94733_Donor 3 AD - A_adipose | 39.2 |
| 94734_Donor 3 AD - B_adipose | 11.4 |
| 94735_Donor 3 AD - C_adipose | 34.4 |
| 77138_Liver_HepG2untreated | 8.4 |
| 73556_Heart_Cardiac stromal cells (primary) | 0.0 |
| 81735_Small Intestine | 100.0 |
| 72409_Kidney_Proximal Convoluted Tubule | 9.9 |
| 82685_Small intestine_Duodenum | 70.2 |
| 90650_Adrenal_Adrenocortical adenoma | 25.5 |
| 72410_Kidney_HRCE | 10.4 |
| 72411_Kidney_HRE | 7.2 |
| 73139_Uterus_Uterine smooth muscle cells | 0.0 |

TABLE APH
general oncology screening panel_v_2.4

| | Rel. Exp. (%) |
|---|---|
| | Ag1688, Run |
| Tissue Name | 260552690 |
| Colon cancer 1 | 1.8 |
| Colon cancer NAT 1 | 1.0 |
| Colon cancer 2 | 0.4 |
| Colon cancer NAT 2 | 1.2 |
| Colon cancer 3 | 0.8 |
| Colon cancer NAT 3 | 2.5 |
| Colon malignant cancer 4 | 2.1 |
| Colon normal adjacent tissue 4 | 0.2 |
| Lung cancer 1 | 0.2 |
| Lung NAT 1 | 0.2 |
| Lung cancer 2 | 1.0 |
| Lung NAT 2 | 0.8 |
| Squamous cell carcinoma 3 | 0.5 |
| Lung NAT 3 | 0.0 |
| metastatic melanoma 1 | 1.1 |
| Melanoma 2 | 0.1 |
| Melanoma 3 | 0.0 |
| metastatic melanoma 4 | 2.0 |
| metastatic melanoma 5 | 3.0 |
| Bladder cancer 1 | 0.6 |
| Bladder cancer NAT 1 | 0.0 |
| Bladder cancer 2 | 0.3 |
| Bladder cancer NAT 2 | 0.1 |
| Bladder cancer NAT 3 | 0.0 |
| Bladder cancer NAT 4 | 1.1 |
| Prostate adenocarcinoma 1 | 3.7 |
| Prostate adenocarcinoma 2 | 0.2 |
| Prostate adenocarcinoma 3 | 1.2 |
| Prostate adenocarcinoma 4 | 3.5 |
| Prostate cancer NAT 5 | 0.6 |
| Prostate adenocarcinoma 6 | 0.2 |
| Prostate adenocarcinoma 7 | 0.0 |
| Prostate adenocarcinoma 8 | 0.0 |
| Prostate adenocarcinoma 9 | 0.0 |
| Prostate cancer NAT 10 | 0.1 |
| Kidney cancer 1 | 7.7 |
| Kidney NAT 1 | 5.7 |
| Kidney cancer 2 | 40.1 |
| Kidney NAT 2 | 23.8 |
| Kidney cancer 3 | 100.0 |
| Kidney NAT 3 | 5.6 |
| Kidney cancer 4 | 2.0 |
| Kidney NAT 4 | 4.2 |

is also seen in samples derived from orthoarthitis/ rheumatoid arthritis bone, cartilage, synovium and synovial fluid samples, from normal lung, COPD lung, emphysema, atopic asthma, asthma, Crohn's disease (normal matched control and diseased), ulcerative colitis(normal matched control and diseased), and psoriasis (normal matched control and diseased). Therefore, therapeutic modulation of this gene product may ameliorate symptoms/conditions associated with autoimmune and inflammatory disorders including psoriasis, asthma, inflammatory bowel disease, rheumatoid arthritis and osteoarthritis.

CNS_neurodegeneration_v1.0 Summary: Ag1688 This panel confirms the expression of this gene at low levels in the brains of an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel 1.3D Summary: Ag1688 Expression of this gene, a plasma kallikrein, is significantly higher in liver (CTs=28) than in any other sample on this panel. Thus, expression of this gene could be used as a marker of liver tissue. In addition, low levels of expression of this gene is also detected in tissues with metabolic/endocrine functions including pancreas, adrenal gland, thyroid, pituitary gland, skeletal muscle, heart, and the gastrointestinal tract. Plasma prekallikrein is a glycoprotein that participates in the surface-dependent activation of blood coagulation, fibrinolysis, kinin generation and inflammation. It is synthesized in the liver and secreted into the blood as a single polypeptide chain. It is converted to plasma kallikrein by factor XlIa. Recently, plasma kallikrein has been implicated in adipose differentiation by remodeling of the fibronectin-rich ECM of preadipocytes. Plg−/− mice show a reduction of fat deposit (Ref. 1, 2). At Curagen, it was found that plasma kallikrein significantly down-regulated in the liver of mice with ‘lean’ phenotype. Thus, based on Curagen GeneCalling data it is hypothesized that plasma kallikrein might cause disruption of adipose differentiation thus leading to obesity if over expressed and to a leaner phenotype if expression is below normal. Therefore, an antagonist to this gene product in the form of small molecule or antibody may be beneficial in the treatment of obesity.

Moderate to low levels of expression of this gene is also seen levels in some of the regions of central nervous system examined, including amygdala, hippocampus, substantia nigra, thalamus, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

References:

1. Hoover-Plow J, Yuen L. Plasminogen binding is increased with adipocyte differentiation. Biochem.Biophys.Res.Commun. (2001) 284, 389-394. PMID: 11394891.

Selvarajan S, Lund L R, Takeuchi T, Craik C S, Werb Z. A plasma kallikrein-dependent plasminogen cascade required for adipocyte differentiation. Nature Cell Biol. (2001) 3, 267-275. PMID: 11231576

Panel 2D Summary: Ag1688 The expression of the CG56155-01 gene appears to be highest in a sample derived from a sample of normal liver tissue adjacent to a metastatic colon cancer CT=26.2). In addition, there is substantial expression in other samples of normal liver, and to a much lesser degree, malignant liver tissue. This liver specific expression is consistent with the expression seen in Panel 1.3D. Thus, the expression of this gene could be used to distinguish liver derived tissue from the toher samples in the panel, and more specifically the expression of this gene could be used to distinguish normal liver from malignant liver tissue. Moreover, therapeutic modulation of this gene, through the use of small molecule drugs, protein therapeutics or antibodies might be of benefit in the treatment of liver cancer.

Panel 4.1D Summary: Ag1688 Highest expression of this gene is detected in liver cirrhosis (CT=31.8). In addition, moderate to low levels of expression of this gene in IL-2 treated NK cells, CD40L and IL-4 treated B lymphocytes and normal kidney. Therefore, therapeutic modulation of the protein encoded for by this gene may be useful in the treatment of inflammatory or autoimmune diseases which effect the liver and kidney including liver cirrhosis and fibrosis, lupus erythematosus and glomerulonephritis.

Panel 5 Islet Summary: Ag1688 Expression of the CG56155-01 gene is limited to pancreatic islets and small intestines. Please see Panel 1.3 for discussion of utility of this gene in metabolic disease.

General oncology screening panel_v_{—}2.4 Summary: Ag1688 Highest expression of this gene is detected in kidney cancer (CT=28.4). Higher expression of this gene is associated with cancer compared to normal kidney. Therefore, expression of this gene may be used as diagnostic marker for kidney cancer and therapeutic modulation of this gene or protein encoded by this gene may through the use of antibodies or small molecule drug may be useful in the treatment of kidney cancer.

AQ. CG59595-01: Ribonuclease 6 Precursor.

Expression of gene CG59595-01 was assessed using the primer-probe set Ag3488, described in Table AQA. Results of the RTQ-PCR runs are shown in Tables AQB, AQC, AQD, AQE, AQF and AQG.

TABLE AQA
Probe Name Ag3488

| | | | Start | SEQ ID |
|---|---|---|---|---|
| Primers | Sequences | Length | Position | No |
| Forward | 5′-aactgtgcctcactaagcaaga-3′ | 22 | 963 | 609 |
| Probe | TET-5′-agcagctgcaaaactgcaccgag-3′-TAMRA | 23 | 987 | 610 |
| Reverse | 5′-catttgccagccagacttc-3′ | 19 | 1037 | 611 |

TABLE AQB
CNS_neurodegeneration_v1.0

| | Rel. Exp. (%) |
|---|---|
| | Ag3488, Run |
| Tissue Name | 206533698 |
| AD 1 Hippo | 54.0 |
| AD 2 Hippo | 72.7 |
| AD 3 Hippo | 34.2 |
| AD 4 Hippo | 34.4 |
| AD 5 hippo | 74.7 |
| AD 6 Hippo | 70.2 |
| Control 2 Hippo | 63.3 |
| Control 4 Hippo | 47.6 |
| Control (Path) 3 Hippo | 11.3 |
| AD 1 Temporal Ctx | 43.5 |
| AD 2 Temporal Ctx | 42.0 |
| AD 3 Temporal Ctx | 25.9 |
| AD 4 Temporal Ctx | 37.6 |
| AD 5 Inf Temporal Ctx | 93.3 |
| AD 5 Sup Temporal Ctx | 100.0 |
| AD 6 Inf Temporal Ctx | 74.7 |
| AD 6 Sup Temporal Ctx | 56.3 |
| Control 1 Temporal Ctx | 15.6 |
| Control 2 Temporal Ctx | 57.8 |
| Control 3 Temporal Ctx | 29.3 |
| Control 4 Temporal Ctx | 24.8 |
| Control (Path) 1 Temporal Ctx | 62.0 |
| Control (Path) 2 Temporal Ctx | 29.5 |
| Control (Path) 3 Temporal Ctx | 8.8 |
| Control (Path) 4 Temporal Ctx | 42.6 |
| AD 1 Occipital Ctx | 36.3 |
| AD 2 Occipital Ctx (Missing) | 0.0 |
| AD 3 Occipital Ctx | 20.7 |
| AD 4 Occipital Ctx | 31.4 |
| AD 5 Occipital Ctx | 22.1 |
| AD 6 Occipital Ctx | 42.6 |
| Control 1 Occipital Ctx | 7.1 |
| Control 2 Occipital Ctx | 47.3 |
| Control 3 Occipital Ctx | 21.6 |
| Control 4 Occipital Ctx | 18.3 |
| Control (Path) 1 Occipital Ctx | 63.7 |
| Control (Path) 2 Occipital Ctx | 15.2 |
| Control (Path) 3 Occipital Ctx | 5.2 |
| Control (Path) 4 Occipital Ctx | 27.4 |
| Control 1 Parietal Ctx | 12.5 |
| Control 2 Parietal Ctx | 59.9 |
| Control 3 Parietal Ctx | 25.2 |
| Control (Path) 1 Parietal Ctx | 57.0 |
| Control (Path) 2 Parietal Ctx | 30.4 |
| Control (Path) 3 Parietal Ctx | 3.8 |
| Control (Path) 4 Parietal Ctx | 51.8 |

TABLE AQC
General_screening_panel_v1.4

| | Rel. Exp. (%) |
|---|---|
| | Ag3488, Run |
| Tissue Name | 213390581 |
| Adipose | 4.1 |
| Melanoma* Hs688(A).T | 2.6 |
| Melanoma* Hs688(B).T | 1.6 |
| Melanoma* M14 | 2.1 |
| Melanoma* LOXIMVI | 0.1 |
| Melanoma* SK-MEL-5 | 2.1 |
| Squamous cell carcinoma SCC-4 | 2.1 |
| Testis Pool | 3.3 |
| Prostate ca.* (bone met) PC-3 | 3.1 |
| Prostate Pool | 5.3 |
| Placenta | 2.1 |
| Uterus Pool | 1.7 |
| Ovarian ca. OVCAR-3 | 4.9 |
| Ovarian ca. SK-OV-3 | 27.5 |
| Ovarian ca. OVCAR-4 | 10.7 |
| Ovarian ca. OVCAR-5 | 7.0 |
| Ovarian ca. IGROV-1 | 57.0 |
| Ovarian ca. OVCAR-8 | 1.4 |
| Ovary | 3.2 |
| Breast ca. MCF-7 | 15.3 |
| Breast ca. MDA-MB-231 | 11.8 |
| Breast ca. BT 549 | 5.4 |
| Breast ca. T47D | 13.0 |
| Breast ca. MDA-N | 1.5 |
| Breast Pool | 7.1 |
| Trachea | 7.3 |
| Lung | 2.8 |
| Fetal Lung | 6.8 |
| Lung ca. NCI-N417 | 0.4 |
| Lung ca. LX-1 | 7.3 |
| Lung ca. NCI-H146 | 1.5 |
| Lung ca. SHP-77 | 6.7 |
| Lung ca. A549 | 2.4 |
| Lung ca. NCI-H526 | 1.5 |
| Lung ca. NCI-H23 | 3.6 |
| Lung ca. NCI-H460 | 3.1 |
| Lung ca. HOP-62 | 2.9 |
| Lung ca. NCI-H522 | 3.0 |
| Liver | 0.8 |
| Fetal Liver | 5.9 |
| Liver ca. HepG2 | 37.6 |
| Kidney Pool | 8.9 |
| Fetal Kidney | 5.5 |
| Renal ca. 786-0 | 100.0 |
| Renal ca. A498 | 17.9 |
| Renal ca. ACHN | 1.8 |
| Renal ca. UO-31 | 6.7 |
| Renal ca. TK-10 | 22.8 |
| Bladder | 14.8 |
| Gastric ca. (liver met.) NCI-N87 | 5.8 |
| Gastric ca. KATO III | 22.2 |
| Colon ca. SW-948 | 6.0 |
| Colon ca. SW480 | 6.4 |
| Colon ca.* (SW480 met) SW620 | 3.3 |
| Colon ca. HT29 | 17.1 |
| Colon ca. HCT-116 | 6.3 |
| Colon ca. CaCo-2 | 10.6 |
| Colon cancer tissue | 16.2 |
| Colon ca. SW1116 | 6.8 |
| Colon ca. Colo-205 | 1.0 |
| Colon ca. SW-48 | 6.7 |
| Colon Pool | 5.5 |
| Small Intestine Pool | 6.4 |
| Stomach Pool | 3.6 |
| Bone Marrow Pool | 2.5 |
| Fetal Heart | 1.5 |
| Heart Pool | 1.8 |
| Lymph Node Pool | 6.2 |
| Fetal Skeletal Muscle | 0.9 |
| Skeletal Muscle Pool | 0.9 |
| Spleen Pool | 10.6 |
| Thymus Pool | 12.2 |
| CNS cancer (glio/astro) U87-MG | 4.8 |
| CNS cancer (glio/astro) U-118-MG | 2.2 |
| CNS cancer (neuro; met) SK-N-AS | 1.7 |
| CNS cancer (astro) SF-539 | 0.3 |
| CNS cancer (astro) SNB-75 | 1.8 |
| CNS cancer (glio) SNB-19 | 47.3 |
| CNS cancer (glio) SF-295 | 7.9 |
| Brain (Amygdala) Pool | 3.9 |
| Brain (cerebellum) | 2.6 |
| Brain (fetal) | 2.5 |
| Brain (Hippocampus) Pool | 2.1 |
| Cerebral Cortex Pool | 2.5 |
| Brain (Substantia nigra) Pool | 3.4 |
| Brain (Thalamus) Pool | 2.8 |
| Brain (whole) | 1.3 |
| Spinal Cord Pool | 6.5 |
| Adrenal Gland | 3.1 |
| Pituitary gland Pool | 1.8 |
| Salivary Gland | 9.5 |
| Thyroid (female) | 7.0 |
| Pancreatic ca. CAPAN2 | 2.6 |
| Pancreas Pool | 13.3 |

TABLE AQD
Panel 2.2

| | Rel. Exp.(%) |
|---|---|
| | Ag3488, Run |
| Tissue Name | 174285071 |
| Normal Colon | 12.2 |
| Colon cancer (OD06064) | 8.0 |
| Colon Margin (OD06064) | 6.6 |
| Colon cancer (OD06159) | 5.3 |
| Colon Margin (OD06159) | 6.7 |
| Colon cancer (OD06297-04) | 4.9 |
| Colon Margin (OD06297-05) | 8.5 |
| CC Gr.2 ascend colon (ODO3921) | 10.4 |
| CC Margin (ODO3921) | 9.0 |
| Colon cancer metastasis (OD06104) | 11.0 |
| Lung Margin (OD06104) | 8.9 |
| Colon mets to lung (OD04451-01) | 19.6 |
| Lung Margin (OD04451-02) | 9.0 |
| Normal Prostate | 11.5 |
| Prostate Cancer (OD04410) | 4.9 |
| Prostate Margin (OD04410) | 4.7 |
| Normal Ovary | 7.3 |
| Ovarian cancer (OD06283-03) | 8.7 |
| Ovarian Margin (OD06283-07) | 4.6 |
| Ovarian Cancer 064008 | 13.7 |
| Ovarian cancer (OD06145) | 12.2 |
| Ovarian Margin (OD06145) | 18.9 |
| Ovarian cancer (OD06455-03) | 80.7 |
| Ovarian Margin (OD06455-07) | 2.4 |
| Normal Lung | 7.9 |
| Invasive poor diff. lung adeno (ODO4945-01 | 14.5 |
| Lung Margin (ODO4945-03) | 8.8 |
| Lung Malignant Cancer (OD03126) | 26.6 |
| Lung Margin (OD03126) | 4.8 |
| Lung Cancer (OD05014A) | 7.9 |
| Lung Margin (OD05014B) | 23.3 |
| Lung cancer (OD06081) | 2.8 |
| Lung Margin (OD06081) | 4.0 |
| Lung Cancer (OD04237-01) | 6.0 |
| Lung Margin (OD04237-02) | 19.6 |
| Ocular Melanoma Metastasis | 4.6 |
| Ocular Melanoma Margin (Liver) | 10.0 |
| Melanoma Metastasis | 6.9 |
| Melanoma Margin (Lung) | 10.2 |
| Normal Kidney | 2.9 |
| Kidney Ca, Nuclear grade 2 (OD04338) | 12.2 |
| Kidney Margin (OD04338) | 9.0 |
| Kidney Ca Nuclear grade 1/2 (OD04339) | 22.7 |
| Kidney Margin (OD04339) | 3.3 |
| Kidney Ca, Clear cell type (OD04340) | 17.8 |
| Kidney Margin (OD04340) | 8.0 |
| Kidney Ca, Nuclear grade 3 (OD04348) | 5.8 |
| Kidney Margin (OD04348) | 21.5 |
| Kidney malignant cancer (OD06204B) | 11.8 |
| Kidney normal adjacent tissue (OD06204E) | 4.9 |
| Kidney Cancer (OD04450-01) | 100.0 |
| Kidney Margin (OD04450-03) | 4.8 |
| Kidney Cancer 8120613 | 0.9 |
| Kidney Margin 8120614 | 3.1 |
| Kidney Cancer 9010320 | 23.7 |
| Kidney Margin 9010321 | 2.4 |
| Kidney Cancer 8120607 | 12.1 |
| Kidney Margin 8120608 | 3.0 |
| Normal Uterus | 11.3 |
| Uterine Cancer 064011 | 16.4 |
| Normal Thyroid | 5.1 |
| Thyroid Cancer 064010 | 4.9 |
| Thyroid Cancer A302152 | 8.7 |
| Thyroid Margin A302153 | 6.5 |
| Normal Breast | 9.9 |
| Breast Cancer (OD04566) | 5.7 |
| Breast Cancer 1024 | 10.8 |
| Breast Cancer (OD04590-01) | 39.8 |
| Breast Cancer Mets (OD04590-03) | 8.8 |
| Breast Cancer Metastasis (OD04655-05) | 9.2 |
| Breast Cancer 064006 | 10.0 |
| Breast Cancer 9100266 | 7.8 |
| Breast Margin 9100265 | 5.0 |
| Breast Cancer A209073 | 6.0 |
| Breast Margin A2090734 | 10.2 |
| Breast cancer (OD06083) | 18.6 |
| Breast cancer node metastasis (OD06083) | 16.6 |
| Normal Liver | 8.0 |
| Liver Cancer 1026 | 5.0 |
| Liver Cancer 1025 | 18.4 |
| Liver Cancer 6004-T | 12.8 |
| Liver Tissue 6004-N | 11.0 |
| Liver Cancer 6005-T | 9.7 |
| Liver Tissue 6005-N | 19.9 |
| Liver Cancer 064003 | 11.4 |
| Normal Bladder | 11.6 |
| Bladder Cancer 1023 | 6.1 |
| Bladder Cancer A302173 | 12.0 |
| Normal Stomach | 23.5 |
| Gastric Cancer 9060397 | 3.0 |
| Stomach Margin 9060396 | 12.7 |
| Gastric Cancer 9060395 | 8.0 |
| Stomach Margin 9060394 | 26.4 |
| Gastric Cancer 064005 | 6.3 |

TABLE AQE
Panel 3D

| | Rel. Exp. (%) |
|---|---|
| | Ag3488, Run |
| Tissue Name | 182098858 |
| Daoy- Medulloblastoma | 1.7 |
| TE671- Medulloblastoma | 10.2 |
| D283 Med- Medulloblastoma | 34.6 |
| PFSK-1- Primitive Neuroectodermal | 11.9 |
| XF-498- CNS | 3.5 |
| SNB-78- Glioma | 21.5 |
| SF-268- Glioblastoma | 11.9 |
| T98G- Glioblastoma | 5.3 |
| SK-N-SH- Neuroblastoma (metastasis) | 22.5 |
| SF-295- Glioblastoma | 10.4 |
| Cerebellum | 11.0 |
| Cerebellum | 9.3 |
| NCI-H292- Mucoepidennoid lung carcinoma | 57.8 |
| DMS-114- Small cell lung cancer | 0.6 |
| DMS-79- Small cell lung cancer | 70.2 |
| NCI-H146- Small cell lung cancer | 20.0 |
| NCI-H526- Small cell lung cancer | 35.6 |
| NCI-N417- Small cell lung cancer | 3.7 |
| NCI-H82- Small cell lung cancer | 6.6 |
| NCI-H157- Squamous cell lung cancer | 0.8 |
| (metastasis) | |
| NCI-H1155- Large cell lung cancer | 15.3 |
| NCI-H1299- Large cell lung cancer | 14.5 |
| NCI-H727- Lung carcinoid | 25.0 |
| NCI-UMC-11- Lung carcinoid | 31.2 |
| LX-1- Small cell lung cancer | 30.6 |
| Colo-205- Colon cancer | 15.1 |
| KM12- Colon cancer | 24.7 |
| KM20L2- Colon cancer | 33.0 |
| NCI-H716- Colon cancer | 24.1 |
| SW-48- Colon adenocarcinoma | 52.9 |
| SW1116- Colon adenocarcinoma | 50.0 |
| LS 174T- Colon adenocarcinoma | 78.5 |
| SW-948- Colon adenocarcinoma | 5.5 |
| SW-480- Colon adenocarcinoma | 25.9 |
| NCI-SNU-5- Gastric carcinoma | 15.2 |
| KATO III- Gastric carcinoma | 66.0 |
| NCI-SNU-16- Gastric carcinoma | 20.6 |
| NCI-SNU-1- Gastric carcinoma | 85.3 |
| RF-1- Gastric adenocarcinoma | 64.2 |
| RF-48- Gastric adenocarcinoma | 70.2 |
| MKN-45- Gastric carcinoma | 33.9 |
| NCI-N87- Gastric carcinoma | 28.5 |
| OVCAR-5- Ovarian carcinoma | 11.5 |
| RL95-2- Uterine carcinoma | 15.7 |
| HelaS3- Cervical adenocarcinoma | 10.5 |
| Ca Ski- Cervical epidermoid carcinoma | 18.6 |
| (metastasis) | |
| ES-2- Ovarian clear cell carcinoma | 10.2 |
| Ramos- Stimulated with PMA/ionomycin 6h | 7.3 |
| Ramos- Stimulated with PMA/ionomycin 14h | 27.7 |
| MEG-01 - Chronic myelogenous leukemia | 27.2 |
| (megokaryoblast) | |
| Raji- Burkitt's lymphoma | 16.0 |
| Daudi- Burkitt's lymphoma | 8.8 |
| U266- B-cell plasmacytoma | 17.3 |
| CA46- Burkitt's lymphoma | 6.4 |
| RL- non-Hodgkin's B-cell lymphoma | 2.9 |
| JM1- pre-B-cell lymphoma | 5.7 |
| Jurkat- T cell leukemia | 5.7 |
| TF-1 - Erythroleukemia | 62.0 |
| HUT 78- T-cell lymphoma | 29.7 |
| U937- Histiocytic lymphoma | 86.5 |
| KU-812- Myelogenous leukemia | 87.1 |
| 769-P- Clear cell renal carcinoma | 8.8 |
| Caki-2- Clear cell renal carcinoma | 26.2 |
| SW 839- Clear cell renal carcinoma | 70.7 |
| G401- Wilms' tumor | 10.2 |
| Hs766T- Pancreatic carcinoma (LN metastasis) | 33.9 |
| CAPAN-1- Pancreatic adenocarcinoma | 15.7 |
| (liver metastasis) | |
| SU86.86- Pancreatic carcinoma | 100.0 |
| (liver metastasis) | |
| BxPC-3- Pancreatic adenocarcinoma | 10.9 |
| HP AC- Pancreatic adenocarcinoma | 5.8 |
| MIA PaCa-2- Pancreatic carcinoma | 0.1 |
| CFPAC-1- Pancreatic ductal adenocarcinoma | 37.6 |
| PANC-1- Pancreatic epithelioid | 2.9 |
| ductal carcinoma | |
| T24- Bladder carcinma (transitional cell) | 12.4 |
| 5637- Bladder carcinoma | 9.0 |
| HT-1197- Bladder carcinoma | 46.0 |
| UM-UC-3- Bladder carcinma (transitional cell) | 5.5 |
| A204- Rhabdomyosarcoma | 8.8 |
| HT-1080- Fibrosarcoma | 10.4 |
| MG-63- Osteosarcoma | 6.7 |
| SK-LMS-1- Leiomyosarcoma (vulva) | 13.2 |
| SJRH30- Rhabdomyosarcoma (met to bone marrow) | 4.7 |
| A431- Epidermoid carcinoma | 12.1 |
| WM266-4- Melanoma | 6.2 |
| DU 145- Prostate carcinoma (brain metastasis) | 0.0 |
| MDA-MB-468- Breast adenocarcinoma | 6.7 |
| SCC-4- Squamous cell carcinoma of tongue | 0.9 |
| SCC-9- Squamous cell carcinoma of tongue | 10.5 |
| SCC-15- Squamous cell carcinoma of tongue | 0.6 |
| CAL 27- Squamous cell carcinoma of tongue | 27.4 |

TABLE AQF
Panel 4D

| | Rel. Exp. (%) |
|---|---|
| | Ag3488, Run |
| Tissue Name | 166441742 |
| Secondary Th1 act | 18.7 |
| Secondary Th2 act | 25.2 |
| Secondary Tr1 act | 29.5 |
| Secondary Th1 rest | 37.9 |
| Secondary Th2 rest | 21.3 |
| Secondary Tr1 rest | 29.3 |
| Primary Th1 act | 7.1 |
| Primary Th2 act | 20.4 |
| Primary Tr1 act | 25.9 |
| Primary Th1 rest | 95.9 |
| Primary Th2 rest | 55.1 |
| Primary Tr1 rest | 28.5 |
| CD45RA CD4 lymphocyte act | 8.8 |
| CD45RO CD4 lymphocyte act | 25.2 |
| CD8 lymphocyte act | 12.6 |
| Secondary CD8 lymphocyte rest | 31.2 |
| Secondary CD8 lymphocyte act | 7.6 |
| CD4 lymphocyte none | 50.3 |
| 2ry Th1/Th2/Tr1_anti-CD95 CH11 | 41.8 |
| LAK cells rest | 23.2 |
| LAK cells IL-2 | 33.9 |
| LAK cells IL-2 + IL-12 | 26.4 |
| LAK cells IL-2 + IFN gamma | 42.9 |
| LAK cells IL-2 + IL-18 | 24.0 |
| LAK cells PMA/ionomycin | 14.3 |
| NK Cells IL-2 rest | 14.2 |
| Two Way MLR 3 day | 39.8 |
| Two Way MLR 5 day | 18.7 |
| Two Way MLR 7 day | 16.6 |
| PBMC rest | 45.1 |
| PBMC PWM | 17.2 |
| PBMC PHA-L | 19.8 |
| Ramos (B cell) none | 23.8 |
| Ramos (B cell) ionomycin | 18.0 |
| B lymphocytes PWM | 21.8 |
| B lymphocytes CD40L and IL-4 | 43.2 |
| EOL-1 dbcAMP | 53.6 |
| EOL-1 dbcAMP PMA/ionomycin | 25.0 |
| Dendritic cells none | 72.2 |
| Dendritic cells LPS | 29.1 |
| Dendritic cells anti-CD40 | 80.7 |
| Monocytes rest | 100.0 |
| Monocytes LPS | 11.0 |
| Macrophages rest | 92.0 |
| Macrophages LPS | 26.8 |
| HUVEC none | 11.0 |
| HUVEC starved | 9.7 |
| HUVEC IL-1beta | 2.6 |
| HUVEC IFN gamma | 3.0 |
| HUVEC TNF alpha + IFN gamma | 3.2 |
| HUVEC TNF alpha + IL4 | 3.6 |
| HUVEC IL-11 | 3.5 |
| Lung Microvascular EC none | 9.2 |
| Lung Microvascular EC TNFalpha + IL-1beta | 7.5 |
| Microvascular Dermal EC none | 9.0 |
| Microsvasular Dermal EC TNFalpha + IL-1beta | 4.4 |
| Bronchial epithelium TNFalpha + IL1beta | 6.5 |
| Small airway epithelium none | 6.0 |
| Small airway epithelium TNFalpha + IL-1beta | 25.3 |
| Coronery artery SMC rest | 7.5 |
| Coronery artery SMC TNFalpha + IL-1beta | 4.0 |
| Astrocytes rest | 5.5 |
| Astrocytes TNFalpha + IL-1beta | 12.2 |
| KU-812 (Basophil) rest | 41.5 |
| KU-812 (Basophil) PMA/ionomycin | 91.4 |
| CCD1106 (Keratinocytes) none | 3.6 |
| CCD1106 (Keratinocytes) TNFalpha + IL-1beta | 7.1 |
| Liver cirrhosis | 25.3 |
| Lupus kidney | 21.6 |
| NCI-H292 none | 46.0 |
| NCI-H292 IL-4 | 43.8 |
| NCI-H292 IL-9 | 51.1 |
| NCI-H292 IL-1 3 | 26.2 |
| NCI-H292 IFN gamma | 23.5 |
| HPAEC none | 3.8 |
| HPAEC TNF alpha + IL-1 beta | 10.2 |
| Lung fibroblast none | 7.3 |
| Lung fibroblast TNF alpha + IL-1 beta | 11.7 |
| Lung fibroblast IL-4 | 6.8 |
| Lung fibroblast IL-9 | 4.6 |
| Lung fibroblast IL-13 | 4.8 |
| Lung fibroblast IFN gamma | 5.7 |
| Dermal fibroblast CCD1070 rest | 8.7 |
| Dermal fibroblast CCD1070 TNF alpha | 20.9 |
| Dermal fibroblast CCD1070 IL-1 beta | 3.3 |
| Dermal fibroblast IFN gamma | 3.2 |
| Dermal fibroblast IL-4 | 7.0 |
| IBD Colitis 2 | 17.6 |
| IBD Crohn's | 11.4 |
| Colon | 93.3 |
| Lung | 27.4 |
| Thymus | 17.6 |
| Kidney | 56.6 |

TABLE AQG
general oncology screening panel_v_2.4

| | Rel. Exp. (%) |
|---|---|
| | Ag3488, Run |
| Tissue Name | 259737914 |
| Colon cancer 1 | 6.9 |
| Colon cancer NAT 1 | 2.9 |
| Colon cancer 2 | 4.4 |
| Colon cancer NAT 2 | 2.6 |
| Colon cancer 3 | 27.4 |
| Colon cancer NAT 3 | 3.5 |
| Colon malignant cancer 4 | 12.6 |
| Colon normal adjacent tissue 4 | 1.1 |
| Lung cancer 1 | 2.7 |
| Lung NAT 1 | 0.5 |
| Lung cancer 2 | 11.8 |
| Lung NAT 2 | 0.6 |
| Squamous cell carcinoma 3 | 5.8 |
| Lung NAT 3 | 0.2 |
| metastatic melanoma 1 | 3.2 |
| Melanoma 2 | 0.8 |
| Melanoma 3 | 0.7 |
| metastatic melanoma 4 | 6.2 |
| metastatic melanoma 5 | 4.7 |
| Bladder cancer 1 | 0.7 |
| Bladder cancer NAT 1 | 0.0 |
| Bladder cancer 2 | 0.9 |
| Bladder cancer NAT 2 | 0.3 |
| Bladder cancer NAT 3 | 0.2 |
| Bladder cancer NAT 4 | 0.8 |
| Prostate adenocarcinoma 1 | 4.2 |
| Prostate adenocarcinoma 2 | 0.8 |
| Prostate adenocarcinoma 3 | 1.8 |
| Prostate adenocarcinoma 4 | 6.7 |
| Prostate cancer NAT 5 | 2.7 |
| Prostate adenocarcinoma 6 | 1.7 |
| Prostate adenocarcinoma 7 | 2.4 |
| Prostate adenocarcinoma 8 | 0.6 |
| Prostate adenocarcinoma 9 | 3.0 |
| Prostate cancer NAT 10 | 0.3 |
| Kidney cancer 1 | 11.3 |
| Kidney NAT 1 | 1.1 |
| Kidney cancer 2 | 55.1 |
| Kidney NAT 2 | 2.8 |
| Kidney cancer 3 | 100.0 |
| Kidney NAT 3 | 0.6 |
| Kidney cancer 4 | 31.6 |
| Kidney NAT 4 | 0.8 |

CNS_neurodegeneration_v1.0 Summary: Ag3488 This panel does not show differential expression of this gene in Alzheimer's disease. However, this profile confirms the expression of this gene at moderate levels in the brain. Please see Panel 1.4 for discussion of utility of this gene in the central nervous system.

General_screening_panel_v1.4 Summary: Ag3488 Highest expression of this gene is seen in a renal cancer cell line (CT=23.2). This gene is widely expressed in this panel, with high to moderate levels of expression seen in brain, colon, gastric, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at high to moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

This gene is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 2.2 Summary: Ag3488 Highest expression is seen in a kidney cancer (CT=28). In addition, this gene is more highly expressed in kidney cancer than in the corresponding normal adjacent tissue. Thus, expression of this gene could be used as a marker of this cancer. Furthemore, therapeutic modulation of the expression or function of this gene product may be useful in the treatment of kidney cancer.

Panel 3D Summary: Ag3488 Highest expression is seen in a pancreatic cancer cell line (CT=29.6). Moderate levels of expression are also seen in many cancer cell lines on this panel. Please see Panel 1.4 for discussion of utility of this gene in cancer.

Panel 4D Summary: Ag3488 Highest expression is seen in resting monocytes (CT=25.3). This gene is also expressed at moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

General oncology screening_panel_v_{—}2.4 Summary: Ag3488 Highest expression is seen in kidney cancer (CT=23.2). In addition, this gene is more highly expressed in colon and kidney cancer than in the corresponding normal adjacent tissue. Thus, expression of this gene could be used as a marker of these cancers. Furthemore, therapeutic modulation of the expression or function of this gene product may be useful in the treatment of colon and kidney cancer.

AR. CG92142-01: Glycerol-3-Phosphate Acyltransferase.

Expression of gene CG92142-01 was assessed using the primer-probe set Ag3774, described in Table ARA. Results of the RTQ-PCR runs are shown in Tables ARB, ARC, ARD, ARE and ARF.

TABLE ARA
Probe Name Ag3774

| | | | Start | SEQ ID |
|---|---|---|---|---|
| Primers | Sequences | Length | Position | No |
| Forward | 5′-ggtgctgctaaaactgttcaac-3′ | 22 | 673 | 612 |
| Probe | TET-5′-tggaacattcaaattcacaaaggtca-3′-TAMRA | 26 | 704 | 613 |
| Reverse | 5′-attcgtctcagttgcagcttt-3′ | 21 | 743 | 614 |

TABLE ARB
CNS_neurodegeneration_v1.0

| | Rel. Exp. (%) |
|---|---|
| | Ag3774, Run |
| Tissue Name | 206871268 |
| AD 1 Hippo | 29.1 |
| AD 2 Hippo | 73.7 |
| AD 3 Hippo | 10.0 |
| AD 4 Hippo | 14.6 |
| AD 5 Hippo | 92.0 |
| AD 6 Hippo | 45.1 |
| Control 2 Hippo | 44.1 |
| Control 4 Hippo | 20.3 |
| Control (Path) 3 Hippo | 19.9 |
| AD 1 Temporal Ctx | 20.6 |
| AD 2 Temporal Ctx | 75.3 |
| AD 3 Temporal Ctx | 13.4 |
| AD 4 Temporal Ctx | 45.1 |
| AD 5 Inf Temporal Ctx | 100.0 |
| AD 5 Sup Temporal Ctx | 78.5 |
| AD 6 Inf Temporal Ctx | 43.5 |
| AD 6 Sup Temporal Ctx | 50.7 |
| Control 1 Temporal Ctx | 25.5 |
| Control 2 Temporal Ctx | 46.7 |
| Control 3 Temporal Ctx | 57.0 |
| Control 3 Temporal Ctx | 25.2 |
| Control (Path) 1 Temporal Ctx | 66.4 |
| Control (Path) 2 Temporal Ctx | 52.1 |
| Control (Path) 3 Temporal Ctx | 29.3 |
| Control (Path) 4 Temporal Ctx | 50.3 |
| AD 1 Occipital Ctx | 22.4 |
| AD 2 Occipital Ctx (Missing) | 0.0 |
| AD 3 Occipital Ctx | 20.3 |
| AD 4 Occipital Ctx | 33.9 |
| AD 5 Occipital Ctx | 37.6 |
| AD 6 Occipital Ctx | 24.7 |
| Control 1 Occipital Ctx | 11.3 |
| Control 2 Occipital Ctx | 48.0 |
| Control 3 Occipital Ctx | 43.5 |
| Control 4 Occipital Ctx | 21.2 |
| Control (Path) 1 Occipital Ctx | 81.8 |
| Control (Path) 2 Occipital Ctx | 12.9 |
| Control (Path) 3 Occipital Ctx | 13.6 |
| Control (Path) 4 Occipital Ctx | 45.1 |
| Control 1 Parietal Ctx | 25.2 |
| Control 2 Parietal Ctx | 84.7 |
| Control 3 Parietal Ctx | 41.2 |
| Control (Path) 1 Parietal Ctx | 91.4 |
| Control (Path) 2 Parietal Ctx | 38.2 |
| Control (Path) 3 Parietal Ctx | 19.1 |
| Control (Path) 4 Parietal Ctx | 48.0 |

TABLE ARC
General_screening_panel_v1.4

| | Rel. Exp. (%) |
|---|---|
| | Ag3774, Run |
| Tissue Name | 213515543 |
| Adipose | 63.7 |
| Melanoma* Hs688(A).T | 16.0 |
| Melanoma* Hs688(B).T | 74.7 |
| Melanoma* M14 | 10.2 |
| Melanoma* LOXIMVI | 76.8 |
| Melanoma* SK-MEL-5 | 23.8 |
| Squamous cell carcinoma SCC-4 | 5.8 |
| Testis Pool | 12.8 |
| Prostate ca.* (bone met) PC-3 | 10.3 |
| Prostate Pool | 2.3 |
| Placenta | 1.3 |
| Uterus Pool | 1.6 |
| Ovarian ca. OVCAR-3 | 10.6 |
| Ovarian ca. SK-OV-3 | 15.6 |
| Ovarian ca. OVCAR-4 | 5.4 |
| Ovarian ca. OVCAR-5 | 6.3 |
| Ovarian ca. IGROV-1 | 5.5 |
| Ovarian ca. OVCAR-8 | 4.9 |
| Ovary | 4.0 |
| Breast ca. MCF-7 | 11.7 |
| Breast ca. MDA-MB-231 | 8.5 |
| Breast ca. BT 549 | 6.5 |
| Breast ca. T47D | 8.9 |
| Breast ca. MDA-N | 10.7 |
| Breast Pool | 5.0 |
| Trachea | 10.6 |
| Lung | 1.0 |
| Fetal Lung | 6.2 |
| Lung ca. NCI-N417 | 3.2 |
| Lung ca. LX-1 | 9.3 |
| Lung ca. NCI-H146 | 2.9 |
| Lung ca. SHP-77 | 16.2 |
| Lung ca. A549 | 7.6 |
| Lung ca. NCI-H526 | 1.9 |
| Lung ca. NCI-H23 | 12.7 |
| Lung ca. NCI-H460 | 7.7 |
| Lung ca. HOP-62 | 6.0 |
| Lung ca. NCI-H522 | 17.6 |
| Liver | 16.3 |
| Fetal Liver | 70.7 |
| Liver ca. HepG2 | 42.9 |
| Kidney Pool | 8.5 |
| Fetal Kidney | 6.6 |
| Renal ca. 786-0 | 10.3 |
| Renal ca. A498 | 2.5 |
| Renal ca. ACHN | 7.3 |
| Renal ca. UO-31 | 7.2 |
| Renal ca. TK-10 | 21.5 |
| Bladder | 6.3 |
| Gastric ca. (liver met.) NCI-N87 | 9.7 |
| Gastric ca. KATO III | 16.5 |
| Colon ca. SW-948 | 3.3 |
| Colon ca. SW480 | 12.9 |
| Colon ca.* (SW480 met) SW620 | 8.6 |
| Colon ca. HT29 | 4.1 |
| Colon ca. HCT-116 | 25.3 |
| Colon ca. CaCo-2 | 52.5 |
| Colon cancer tissue | 10.4 |
| Colon ca. SW1116 | 3.0 |
| Colon ca. Colo-205 | 2.9 |
| Colon ca. SW-48 | 2.5 |
| Colon Pool | 4.5 |
| Small Intestine Pool | 5.9 |
| Stomach Pool | 3.3 |
| Bone Marrow Pool | 2.8 |
| Fetal Heart | 3.1 |
| Heart Pool | 4.0 |
| Lymph Node Pool | 7.2 |
| Fetal Skeletal Muscle | 11.0 |
| Skeletal Muscle Pool | 10.9 |
| Spleen Pool | 5.3 |
| Thymus Pool | 7.6 |
| CNS cancer (glio/astro) U87-MG | 9.7 |
| CNS cancer (glio/astro) U-118-MG | 19.1 |
| CNS cancer (neuro; met) SK-N-AS | 22.1 |
| CNS cancer (astro) SF-539 | 5.9 |
| CNS cancer (astro) SNB-75 | 22.5 |
| CNS cancer (glio) SNB-19 | 5.0 |
| CNS cancer (glio) SF-295 | 100.0 |
| Brain (Amygdala) Pool | 2.9 |
| Brain (cerebellum) | 2.4 |
| Brain (fetal) | 17.9 |
| Brain (Hippocampus) Pool | 5.9 |
| Cerebral Cortex Pool | 7.5 |
| Brain (Substantia nigra) Pool | 5.8 |
| Brain (Thalamus) Pool | 8.1 |
| Brain (whole) | 8.4 |
| Spinal Cord Pool | 4.8 |
| Adrenal Gland | 65.5 |
| Pituitary gland Pool | 1.0 |
| Salivary Gland | 3.0 |
| Thyroid (female) | 3.8 |
| Pancreatic ca. CAPAN2 | 5.4 |
| Pancreas Pool | 5.7 |

TABLE ARD
Panel 2.2

| | Rel. Exp. (%) |
|---|---|
| | Ag3774, Run |
| Tissue Name | 174448446 |
| Normal Colon | 7.9 |
| Colon cancer (OD06064) | 4.9 |
| Colon Margin (OD06064) | 3.6 |
| Colon cancer (OD06159) | 0.2 |
| Colon Margin (OD06159) | 2.8 |
| Colon cancer (OD06297-04) | 0.6 |
| Colon Margin (OD06297-05) | 2.3 |
| CC Gr.2 ascend colon (ODO3921) | 0.5 |
| CC Margin (ODO3921) | 1.0 |
| Colon cancer metastasis (OD06104) | 1.6 |
| Lung Margin (OD06104) | 1.1 |
| Colon mets to lung (OD04451-01) | 2.2 |
| Lung Margin (OD04451-02) | 2.3 |
| Normal Prostate | 0.6 |
| Prostate Cancer (OD04410) | 1.2 |
| Prostate Margin (OD04410) | 1.2 |
| Normal Ovary | 1.0 |
| Ovarian cancer (OD06283-03) | 1.0 |
| Ovarian Margin (OD06283-07) | 10.1 |
| Ovarian Cancer 064008 | 3.3 |
| Ovarian cancer (OD06145) | 2.1 |
| Ovarian Margin (OD06145) | 2.4 |
| Ovarian cancer (OD06455-03) | 1.7 |
| Ovarian Margin (OD06455-07) | 1.3 |
| Normal Lung | 3.1 |
| Invasive poor diff. lung adeno (ODO4945-01 | 1.4 |
| Lung Margin (ODO4945-03) | 2.2 |
| Lung Malignant Cancer (OD03126) | 2.0 |
| Lung Margin (OD03126) | 0.7 |
| Lung Cancer (OD05014A) | 1.2 |
| Lung Margin (OD05014B) | 7.1 |
| Lung cancer (OD06081) | 0.1 |
| Lung Margin (OD06081) | 2.0 |
| Lung Cancer (OD04237-01) | 1.0 |
| Lung Margin (OD04237-02) | 2.6 |
| Ocular Melanoma Metastasis | 7.5 |
| Ocular Melanoma Margin (Liver) | 19.5 |
| Melanoma Metastasis | 2.0 |
| Melanoma Margin (Lung) | 3.6 |
| Normal Kidney | 1.6 |
| Kidney Ca, Nuclear grade 2 (OD04338) | 3.3 |
| Kidney Margin (OD04338) | 1.3 |
| Kidney Ca Nuclear grade 1/2 (OD04339) | 2.2 |
| Kidney Margin (OD04339) | 2.2 |
| Kidney Ca, Clear cell type (OD04340) | 0.7 |
| Kidney Margin (OD04340) | 4.0 |
| Kidney Ca, Nuclear grade 3 (OD04348) | 0.9 |
| Kidney Margin (OD04348) | 8.7 |
| Kidney malignant cancer (OD06204B) | 2.2 |
| Kidney normal adjacent tissue (OD06204E) | 0.4 |
| Kidney Cancer (OD04450-01) | 3.4 |
| Kidney Margin (OD04450-03) | 3.3 |
| Kidney Cancer 8120613 | 0.8 |
| Kidney Margin 8120614 | 1.0 |
| Kidney Cancer 9010320 | 1.6 |
| Kidney Margin 9010321 | 0.2 |
| Kidney Cancer 8120607 | 0.8 |
| Kidney Margin 8120608 | 0.3 |
| Normal Uterus | 5.0 |
| Uterine Cancer 064011 | 1.1 |
| Normal Thyroid | 0.3 |
| Thyroid Cancer 064010 | 0.6 |
| Thyroid Cancer A302152 | 2.2 |
| Thyroid Margin A302153 | 2.9 |
| Normal Breast | 61.6 |
| Breast Cancer (OD04566) | 2.7 |
| Breast Cancer 1024 | 4.8 |
| Breast Cancer (OD04590-01) | 4.8 |
| Breast Cancer Mets (OD04590-03) | 30.1 |
| Breast Cancer Metastasis (OD04655-05) | 6.0 |
| Breast Cancer 064006 | 2.0 |
| Breast Cancer 9100266 | 1.5 |
| Breast Margin 9100265 | 3.6 |
| Breast Cancer A209073 | 1.1 |
| Breast Margin A2090734 | 5.8 |
| Breast cancer (OD06083) | 4.2 |
| Breast cancer node metastasis (OD06083) | 12.6 |
| Normal Liver | 87.7 |
| Liver Cancer 1026 | 12.5 |
| Liver Cancer 1025 | 100.0 |
| Liver Cancer 6004-T | 63.7 |
| Liver Tissue 6004-N | 4.8 |
| Liver Cancer 6005-T | 28.5 |
| Liver Tissue 6005-N | 67.8 |
| Liver Cancer 064003 | 12.2 |
| Normal Bladder | 2.3 |
| Bladder Cancer 1023 | 0.3 |
| Bladder Cancer A302173 | 1.4 |
| Normal Stomach | 6.0 |
| Gastric Cancer 9060397 | 0.9 |
| Stomach Margin 9060396 | 1.7 |
| Gastric Cancer 9060395 | 1.9 |
| Stomach Margin 9060394 | 2.3 |
| Gastric Cancer 064005 | 1.9 |

TABLE ARE
Panel 4.1D

| | Rel. Exp. (%) |
|---|---|
| | Ag3774, Run |
| Tissue Name | 170130276 |
| Secondary Th1 act | 39.8 |
| Secondary Th2 act | 44.4 |
| Secondary Tr1 act | 33.7 |
| Secondary Th1 rest | 9.5 |
| Secondary Th2 rest | 11.4 |
| Secondary Tr1 rest | 12.2 |
| Primary Th1 act | 36.6 |
| Primary Th2 act | 39.8 |
| Primary Tr1 act | 28.9 |
| Primary Th1 rest | 24.8 |
| Primary Th2 rest | 11.7 |
| Primary Tr1 rest | 23.2 |
| CD45RA CD4 lymphocyte act | 45.1 |
| CD45RO CD4 lymphocyte act | 45.1 |
| CD8 lymphocyte act | 49.0 |
| Secondary CD8 lymphocyte rest | 31.2 |
| Secondary CD8 lymphocyte act | 22.1 |
| CD4 lymphocyte none | 11.0 |
| 2ry Th1/Th2/Tr1_anti-CD95 CH11 | 15.9 |
| LAK cells rest | 18.7 |
| LAK cells IL-2 | 31.4 |
| LAK cells IL-2 + IL-12 | 25.3 |
| LAK cells IL-2 + IFN gamma | 46.7 |
| LAK cells IL-2 + IL-18 | 32.8 |
| LAK cells PMA/ionomycin | 3.9 |
| NK Cells IL-2 rest | 30.8 |
| Two Way MLR 3 day | 23.3 |
| Two Way MLR 5 day | 37.6 |
| Two Way MLR 7 day | 17.8 |
| PBMC rest | 4.1 |
| PBMC PWM | 35.4 |
| PBMC PHA-L | 20.9 |
| Ramos (B cell) none | 76.8 |
| Ramos (B cell) ionomycin | 68.8 |
| B lymphocytes PWM | 41.2 |
| B lymphocytes CD40L and IL-4 | 28.9 |
| EOL-1 dbcAMP | 17.4 |
| EOL-1 dbcAMP PMA/ionomycin | 20.9 |
| Dendritic cells none | 21.0 |
| Dendritic cells LPS | 5.7 |
| Dendritic cells anti-CD40 | 22.5 |
| Monocytes rest | 7.9 |
| Monocytes LPS | 2.6 |
| Macrophages rest | 22.2 |
| Macrophages LPS | 4.5 |
| HUVEC none | 29.7 |
| HUVEC starved | 34.6 |
| HUVEC IL-1beta | 38.2 |
| HUVEC IFN gamma | 39.0 |
| HUVEC TNF alpha + IFN gamma | 19.1 |
| HUVEC TNF alpha + IL4 | 28.1 |
| HUVEC IL-11 | 25.2 |
| Lung Microvascular EC none | 32.3 |
| Lung Microvascular EC TNFalpha + IL-1beta | 36.3 |
| Microvascular Dermal EC none | 26.4 |
| Microsvasular Dermal EC TNFalpha + IL-1beta | 23.3 |
| Bronchial epithelium TNFalpha + IL1beta | 38.4 |
| Small airway epithelium none | 24.1 |
| Small airway epithelium TNFalpha + IL-1beta | 28.9 |
| Coronery artery SMC rest | 31.4 |
| Coronery artery SMC TNFalpha + IL-1beta | 24.5 |
| Astrocytes rest | 46.3 |
| Astrocytes TNFalpha + IL-1beta | 12.1 |
| KU-812 (Basophil) rest | 37.9 |
| KU-812 (Basophil) PMA/ionomycin | 49.3 |
| CCD1106 (Keratinocytes) none | 56.3 |
| CCD1106 (Keratinocytes) TNFalpha + IL-1beta | 34.6 |
| Liver cirrhosis | 38.4 |
| NCI-H292 none | 25.2 |
| NCI-H292 IL-4 | 36.3 |
| NCI-H292 IL-9 | 47.6 |
| NCI-H292 IL-13 | 37.1 |
| NCI-H292 IFN gamma | 49.3 |
| HPAEC none | 27.7 |
| HPAEC TNF alpha + IL-1 beta | 31.9 |
| Lung fibroblast none | 44.1 |
| Lung fibroblast TNF alpha + IL-1 beta | 17.0 |
| Lung fibroblast IL-4 | 34.9 |
| Lung fibroblast IL-9 | 62.4 |
| Lung fibroblast IL-13 | 42.0 |
| Lung fibroblast IFN gamma | 25.2 |
| Dermal fibroblast CCD1070 rest | 100.0 |
| Dermal fibroblast CCD1070 TNF alpha | 66.4 |
| Dermal fibroblast CCD1070 IL-1 beta | 38.2 |
| Dermal fibroblast IFN gamma | 17.0 |
| Dermal fibroblast IL-4 | 47.3 |
| Dermal Fibroblasts rest | 29.5 |
| Neutrophils TNFa + LPS | 0.0 |
| Neutrophils rest | 2.3 |
| Colon | 15.4 |
| Lung | 23.8 |
| Thymus | 68.3 |
| Kidney | 49.3 |

TABLE ARF
Panel 5D

| | Rel. Exp. (%) |
|---|---|
| | Ag3774, Run |
| Tissue Name | 223675472 |
| 97457_Patient-02go_adipose | 17.7 |
| 97476_Patient-07sk_skeletal muscle | 3.6 |
| 97477_Patient-07ut_uterus | 2.3 |
| 97478_Patient-07pl_placenta | 2.2 |
| 97481_Patient-08sk_skeletal muscle | 6.4 |
| 97482_Patient-08ut_uterus | 1.6 |
| 97483_Patient-08pl_placenta | 0.8 |
| 97486_Patient-09sk_skeletal muscle | 0.5 |
| 97487_Patient-09ut_uterus | 2.1 |
| 97488_Patient-09pl_placenta | 0.8 |
| 97492_Patient-10ut_uterus | 1.6 |
| 97493_Patient-10pl_placenta | 1.4 |
| 97495_Patient-11go_adipose | 10.4 |
| 97496_Patient-11sk_skeletal muscle | 2.8 |
| 97497_Patient-11ut_uterus | 2.1 |
| 97498_Patient-11pl_placenta | 1.8 |
| 97500_Patient-12go_adipose | 13.5 |
| 97501_Patient-12sk_skeletal muscle | 6.0 |
| 97502_Patient-12ut_uterus | 2.6 |
| 97503_Patient-12pl_placenta | 0.4 |
| 94721_Donor 2 U - A_Mesenchymal Stem Cells | 3.5 |
| 94722_Donor 2 U - B_Mesenchymal Stem Cells | 3.7 |
| 94723_Donor 2 U - C_Mesenchymal Stem Cells | 2.7 |
| 94709_Donor 2 AM - A_adipose | 19.6 |
| 94710_Donor 2 AM - B_adipose | 9.3 |
| 94711_Donor 2 AM - C_adipose | 7.5 |
| 94712_Donor 2 AD - A_adipose | 56.6 |
| 94713_Donor 2 AD - B_adipose | 72.2 |
| 94714_Donor 2 AD - C_adipose | 70.2 |
| 94742_Donor 3 U - A_Mesenchymal Stem Cells | 1.6 |
| 94743_Donor 3 U - B_Mesenchymal Stem Cells | 1.8 |
| 94730_Donor 3 AM - A_adipose | 13.1 |
| 94731_Donor 3 AM - B_adipose | 8.5 |
| 94732_Donor 3 AM - C_adipose | 8.7 |
| 94733_Donor 3 AD - A_adipose | 100.0 |
| 94734_Donor 3 AD - B_adipose | 62.9 |
| 94735_Donor 3 AD - C_adipose | 53.2 |
| 77138_Liver_HepG2untreated | 56.6 |
| 73556_Heart_Cardiac stromal cells (primary) | 0.5 |
| 81735_Small Intestine | 2.3 |
| 72409_Kidney_Proximal Convoluted Tubule | 1.0 |
| 82685_Small intestine_Duodenum | 1.6 |
| 90650_Adrenal_Adrenocortical adenoma | 4.6 |
| 72410_Kidney_HRCE | 3.3 |
| 72411_Kidney_HRE | 2.7 |
| 73139_Uterus_Uterine smooth muscle cells | 1.2 |

CNS_neurodegeneration_v1.0 Summary: Ag3774 This panel confirms the expression of the CG92142-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3774 Highest expression of the CG92142-01 gene is detected in CNS cancer (glio) SF-295 cell line (CT=26). High expression of this gene is also in number of cancer cell lines (pancreatic, CNS, colon, gastric, renal, lung, breast, ovarian, squamous cell carcinoma, prostate and melanoma). Therefore, therapeutic modulation of the activity of this gene or its protein product, through the use of small molecule drugs might be beneficial in the treatment of these cancers.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

The CG92142-01 gene codes for mitochondrial glycerol-3-phosphate acyltransferase (GPAT). GPAT is an adipocyte determination and differentiation factor 1 (ADD 1) and sterol regulatory element-binding protein-1 (SREBP- 1) regulated differentiation gene (Ref.1). It is up-regulated by insulin and high-carbohydrate diets (Ref.2). GPAT up-regulation increases triglyceride (TG) synthesis and fat deposition. Inhibition of GPAT activiy could lead to decreased TG synthesis and fat deposition. Troglitazone, a thiazolidinedione compound used to treat non-insulin-dependent diabetes mellitus (NIDDM), was shown to decreases GPAT activity and adipogenesis in ZDF rat islets (ref.3). Therefore, therapeutic modulation of the activity of this gene may prove useful in the treatment of diabetes.

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

References.

1. Ericsson J, Jackson S M, Kim J B, Spiegelman B M, Edwards P A. (1997) Identification of glycerol-3-phosphate acyltransferase as an adipocyte determination and differentiation factor 1- and sterol regulatory element-binding protein-responsive gene. J Biol Chem 272(11):7298-305. PMID: 9054427

2. Dircks L K, Sul H S. (1997) Mammalian mitochondrial glycerol-3-phosphate acyltransferase. Biochim Biophys Acta 1348(1-2):17-26 PMID: 9370312

3. Shimabukuro M, Zhou Y T, Lee Y, Unger R H. (1998) Troglitazone lowers islet fat and restores beta cell function of Zucker diabetic fatty rats. J Biol Chem 273(6):3547-50 PMID: 9452481.

Panel 2.2 Summary: Ag3774 Highest expression of the CG92142-01 gene is detected in liver cancer 1025 sample (CT=28.7). In addition, low to moderate expression of this gene is seen in number of cancer and normal samples used in this panel. Please see Panel 1.4 for a discussion of the potential utility of this gene.

Panel 4.1D Summary: Ag3774 Highest expression of the CG92142-01 gene is detected in resting dermal fibroblast CCD1070 (CT=31). This gene is expressed at low to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Interestingly, expression of this gene is stimulated in PWM treated PBMC cells (CT=32.5) as compared to resting PBMC (35.6). Therefore, expression of this gene can be used to distinguish between resting and stimulated PBMC cells.

Panel 5D Summary: Ag3774 Highest expression of the CG92142-01 gene is detected in 94733_Donor 3 AD-A_adipose sample(CT=27.6). In addition, high to moderated expression of this gene is also seen in number of adipose, small intestine, uterus, skeletal muscle, placenta and mesenchymal stem cell samples. Please see Panel 1.4 for a discussion of the potential utility of this gene.

AS. CG98102-03: Diamine AcetylTransferase.

Expression of gene CG98102-03 was assessed using the primer-probe sets Ag4695, Ag4700, Ag4705 and Ag5877, described in Tables ASA, ASB, ASC and ASD. Results of the RTQ-PCR runs are shown in Tables ASE, ASF and ASG.

TABLE ASA
Probe Name Ag4695

| | | | Start | SEQ ID |
|---|---|---|---|---|
| Primers | Sequences | Length | Position | No |
| Forward | 5′-gccagcctgactgagaaga-3′ | 19 | 968 | 615 |
| Probe | TET-5′-agacgaatgaggaaccacctcctcct-3′-TAMRA | 26 | 929 | 616 |
| Reverse | 5′-caacaatgctgtgtccttcc-3′ | 20 | 658 | 617 |

TABLE ASB
Probe Name Ag4700

| | | | Start | SEQ ID |
|---|---|---|---|---|
| Primers | Sequences | Length | Position | No |
| Forward | 5′-caatctcagatgcagtttgga-3′ | 21 | 174 | 618 |
| Probe | TET-5′-tcagatctttctccttgaatatctttcga-3′-TAMRA | 29 | 142 | 619 |
| Reverse | 5′-agatcacaccaccttgttgttt-3′ | 22 | 119 | 620 |

TABLE ASC
Probe Name Ag4705

| | | | Start | SEQ ID |
|---|---|---|---|---|
| Primers | Sequences | Length | Position | No |
| Forward | 5′-ggctaaatatgaatacatggaag-3′ | 23 | 781 | 621 |
| Probe | TET-5′-ttttggagagcaccccttttaccac-3′-TAMRA | 25 | 716 | 622 |
| Reverse | 5′-atgctgtgtccttccg-3′ | 16 | 663 | 623 |

TABLE ASD
Probe Name Ag5877

| | | | Start | SEQ ID |
|---|---|---|---|---|
| Primers | Sequences | Length | Position | No |
| Forward | 5′-aagaggtgcttctgatctgtcc-3′ | 22 | 428 | 624 |
| Probe | TET-5′tgaagagggttggagactgttcaagatcg-3′-TAMRA | 29 | 397 | 625 |
| Reverse | 5′-catctacagcagcactcctcac-3′ | 22 | 341 | 626 |

TABLE ASE
General_screening_panel_v1.4

| | Rel. | Rel. | Rel. |
|---|---|---|---|
| | Exp. (%) | Exp. (%) | Exp. (%) |
| | Ag4695, | Ag4700, | Ag4705, |
| Tissue | Run | Run | Run |
| Name | 219997539 | 222825527 | 213821747 |
| Adipose | 16.8 | 45.7 | 12.6 |
| Melanoma* | 2.8 | 1.2 | 2.8 |
| Hs688(A).T | | | |
| Melanoma* | 3.1 | 1.3 | 2.0 |
| Hs688(B).T | | | |
| Melanoma* M14 | 25.5 | 13.7 | 18.4 |
| Melanoma* | 1.0 | 0.6 | 1.8 |
| LOXIMVI | | | |
| Melanoma* | 11.9 | 19.5 | 14.2 |
| SK-MEL-5 | | | |
| Squamous cell | 3.1 | 2.3 | 0.8 |
| carcinoma SCC-4 | | | |
| Testis Pool | 5.6 | 3.1 | 4.5 |
| Prostate ca. * | 16.7 | 8.4 | 17.3 |
| (bone met) PC-3 | | | |
| Prostate Pool | 4.9 | 5.5 | 2.2 |
| Placenta | 20.0 | 6.9 | 0.1 |
| Uterus Pool | 1.0 | 11.6 | 0.3 |
| Ovarian ca. | 4.2 | 6.4 | 4.7 |
| OVCAR-3 | | | |
| Ovarian ca. | 7.5 | 8.5 | 9.3 |
| SK-OV-3 | | | |
| Ovarian ca. | 1.7 | 1.2 | 1.5 |
| OVCAR-4 | | | |
| Ovarian ca. | 8.0 | 27.9 | 9.2 |
| OVCAR-5 | | | |
| Ovarian ca. | 32.5 | 83.5 | 40.9 |
| IGROV-1 | | | |
| Ovarian ca. | 9.1 | 20.7 | 4.1 |
| OVCAR-8 | | | |
| Ovary | 5.1 | 5.6 | 4.9 |
| Breast ca. MCF-7 | 1.6 | 3.1 | 2.0 |
| Breast ca. | 2.6 | 10.6 | 2.9 |
| MDA-MB-231 | | | |
| Breast ca. BT 549 | 25.5 | 9.0 | 22.2 |
| Breast ca. T47D | 16.6 | 71.2 | 19.2 |
| Breast ca. MDA-N | 33.4 | 46.7 | 40.9 |
| Breast Pool | 10.4 | 19.3 | 7.5 |
| Trachea | 41.5 | 20.4 | 38.2 |
| Lung | 0.9 | 24.1 | 0.9 |
| Fetal Lung | 80.1 | 82.9 | 65.1 |
| Lung ca. NCI-N417 | 0.2 | 0.1 | 0.3 |
| Lung ca. LX-1 | 50.7 | 82.4 | 53.6 |
| Lung ca. NCI-H146 | 0.6 | 0.3 | 0.8 |
| Lung ca. SHP-77 | 0.8 | 1.8 | 1.2 |
| Lung ca. A549 | 27.2 | 28.1 | 23.7 |
| Lung ca. NCI-H526 | 0.8 | 1.1 | 1.1 |
| Lung ca. NCI-H23 | 43.2 | 100.0 | 66.9 |
| Lung ca. NCI-H460 | 0.6 | 8.5 | 1.0 |
| Lung ca. HOP-62 | 3.6 | 23.8 | 5.1 |
| Lung ca. NCI-H522 | 2.9 | 6.4 | 3.5 |
| Liver | 3.5 | 0.8 | 1.4 |
| Fetal Liver | 20.6 | 5.4 | 14.0 |
| Liver ca. HepG2 | 11.6 | 19.6 | 16.7 |
| Kidney Pool | 6.1 | 36.6 | 0.0 |
| Fetal Kidney | 5.4 | 5.6 | 0.2 |
| Renal ca. 786-0 | 13.3 | 9.0 | 8.1 |
| Renal ca. A498 | 4.9 | 2.4 | 5.8 |
| Renal ca. ACHN | 1.7 | 2.2 | 1.9 |
| Renal ca. UO-31 | 34.6 | 11.2 | 5.1 |
| Renal ca. TK-10 | 9.4 | 14.4 | 11.3 |
| Bladder | 100.0 | 67.4 | 100.0 |
| Gastric ca. (liver | 7.3 | 10.6 | 8.1 |
| met.) NCI-N87 | | | |
| Gastric ca. | 90.8 | 22.8 | 55.9 |
| KATO III | | | |
| Colon ca. SW-948 | 6.3 | 3.4 | 2.0 |
| Colon ca. SW480 | 26.4 | 20.9 | 28.7 |
| Colon ca. * | 35.4 | 50.0 | 38.2 |
| (SW480 met) SW620 | | | |
| Colon ca. HT29 | 3.0 | 4.4 | 3.8 |
| Colon ca. HCT-116 | 21.5 | 27.9 | 31.0 |
| Colon ca. CaCo-2 | 12.9 | 7.5 | 13.8 |
| Colon cancer | 36.3 | 54.0 | 45.4 |
| tissue | | | |
| Colon ca. SW1116 | 0.4 | 1.1 | 1.0 |
| Colon ca. | 13.1 | 4.0 | 5.6 |
| Colo-205 | | | |
| Colon ca. SW-48 | 6.7 | 2.3 | 3.9 |
| Colon Pool | 5.1 | 12.2 | 4.8 |
| Small Intestine | 1.5 | 12.4 | 1.9 |
| Pool | | | |
| Stomach Pool | 24.3 | 31.6 | 17.6 |
| Bone Marrow | 2.3 | 17.7 | 1.4 |
| Pool | | | |
| Fetal Heart | 1.8 | 2.1 | 2.2 |
| Heart Pool | 1.9 | 6.9 | 2.0 |
| Lymph Node Pool | 6.6 | 20.0 | 8.3 |
| Fetal Skeletal | 0.7 | 1.5 | 0.7 |
| Muscle | | | |
| Skeletal | 0.7 | 2.0 | 0.9 |
| Muscle Pool | | | |
| Spleen Pool | 5.2 | 25.3 | 8.7 |
| Thymus Pool | 8.7 | 37.4 | 11.1 |
| CNS cancer (glio/ | 14.9 | 17.7 | 12.6 |
| astro) U87-MG | | | |
| CNS cancer (glio/ | 16.7 | 12.1 | 18.0 |
| astro) U-118-MG | | | |
| CNS cancer | 0.4 | 1.2 | 1.0 |
| (neuro; met) | | | |
| SK-N-AS | | | |
| CNS cancer | 0.6 | 1.3 | 0.9 |
| (astro) SF-539 | | | |
| CNS cancer | 63.3 | 83.5 | 64.6 |
| (astro) SNB-75 | | | |
| CNS cancer | 27.5 | 54.7 | 37.9 |
| (glio) SNB-19 | | | |
| CNS cancer | 50.7 | 72.7 | 66.0 |
| (glio) SF-295 | | | |
| Brain (Amygdala) | 2.9 | 4.9 | 3.5 |
| Pool | | | |
| Brain | 1.1 | 1.0 | 1.2 |
| (cerebellum) | | | |
| Brain (fetal) | 6.0 | 4.2 | 6.0 |
| Brain | 7.8 | 6.7 | 5.7 |
| (Hippocampus) | | | |
| Pool | | | |
| Cerebral | 3.6 | 5.9 | 6.9 |
| Cortex Pool | | | |
| Brain | 5.1 | 6.0 | 7.9 |
| (Substantia | | | |
| nigra) Pool | | | |
| Brain (Thalamus) | 5.7 | 6.5 | 8.6 |
| Pool | | | |
| Brain (whole) | 5.4 | 2.7 | 11.2 |
| Spinal Cord Pool | 6.2 | 10.1 | 7.0 |
| Adrenal Gland | 12.8 | 5.1 | 14.7 |
| Pituitary gland | 2.4 | 2.0 | 4.0 |
| Pool | | | |
| Salivary Gland | 4.1 | 0.9 | 5.4 |
| Thyroid (female) | 23.8 | 10.4 | 5.6 |
| Pancreatic ca. | 8.0 | 10.3 | 9.7 |
| CAPAN2 | | | |
| Pancreas Pool | 11.8 | 21.6 | 17.0 |

TABLE ASF
General_screening_panel_v1.5

| | Rel. Exp. (%) |
|---|---|
| | Ag5877, Run |
| Tissue Name | 248204736 |
| Adipose | 41.2 |
| Melanoma* Hs688(A).T | 3.9 |
| Melanoma* Hs688(B).T | 5.5 |
| Melanoma* M14 | 40.3 |
| Melanoma* LOXIMVI | 1.8 |
| Melanoma* SK-MEL-5 | 20.6 |
| Squamous cell carcinoma SCC-4 | 7.1 |
| Testis Pool | 7.5 |
| Prostate ca.* (bone met) PC-3 | 16.4 |
| Prostate Pool | 17.0 |
| Placenta | 38.2 |
| Uterus Pool | 7.4 |
| Ovarian ca. OVCAR-3 | 6.0 |
| Ovarian ca. SK-OV-3 | 8.8 |
| Ovarian ca. OVCAR-4 | 3.2 |
| Ovarian ca. OVCAR-5 | 22.5 |
| Ovarian ca. IGROV-1 | 67.8 |
| Ovarian ca. OVCAR-8 | 22.1 |
| Ovary | 10.7 |
| Breast ca. MCF-7 | 3.3 |
| Breast ca. MDA-MB-231 | 9.0 |
| Breast ca. BT 549 | 18.3 |
| Breast ca. T47D | 14.2 |
| Breast ca. MDA-N | 33.0 |
| Breast Pool | 13.8 |
| Trachea | 38.2 |
| Lung | 4.1 |
| Fetal Lung | 95.9 |
| Lung ca. NCI-N417 | 0.3 |
| Lung ca. LX-1 | 84.1 |
| Lung ca. NCI-H146 | 0.5 |
| Lung ca. SHP-77 | 1.9 |
| Lung ca. A549 | 43.8 |
| Lung ca. NCI-H526 | 0.7 |
| Lung ca. NCI-H23 | 77.9 |
| Lung ca. NCI-H460 | 9.9 |
| Lung ca. HOP-62 | 5.8 |
| Lung ca. NCI-H522 | 8.6 |
| Liver | 3.3 |
| Fetal Liver | 17.0 |
| Liver ca. HepG2 | 21.3 |
| Kidney Pool | 15.3 |
| Fetal Kidney | 8.5 |
| Renal ca. 786-0 | 8.1 |
| Renal ca. A498 | 6.3 |
| Renal ca. ACHN | 2.6 |
| Renal ca. UO-31 | 32.1 |
| Renal ca. TK-10 | 15.7 |
| Bladder | 100.0 |
| Gastric ca. (liver met.) NCI-N87 | 17.1 |
| Gastric ca. KATO III | 58.2 |
| Colon ca. SW-948 | 6.6 |
| Colon ca. SW480 | 30.8 |
| Colon ca.* (SW480 met) SW620 | 62.4 |
| Colon ca. HT29 | 4.3 |
| Colon ca. HCT-116 | 34.9 |
| Colon ca. CaCo-2 | 12.4 |
| Colon cancer tissue | 59.0 |
| Colon ca. SW1116 | 1.5 |
| Colon ca. Colo-205 | 6.3 |
| Colon ca. SW-48 | 4.2 |
| Colon Pool | 8.4 |
| Small Intestine Pool | 2.4 |
| Stomach Pool | 22.1 |
| Bone Marrow Pool | 6.4 |
| Fetal Heart | 3.4 |
| Heart Pool | 4.5 |
| Lymph Node Pool | 12.7 |
| Fetal Skeletal Muscle | 1.5 |
| Skeletal Muscle Pool | 2.7 |
| Spleen Pool | 20.6 |
| Thymus Pool | 21.0 |
| CNS cancer (glio/astro) U87-MG | 20.9 |
| CNS cancer (glio/astro) U-118-MG | 15.5 |
| CNS cancer (neuro; met) SK-N-AS | 1.5 |
| CNS cancer (astro) SF-539 | 0.9 |
| CNS cancer (astro) SNB-75 | 74.2 |
| CNS cancer (glio) SNB-19 | 80.7 |
| CNS cancer (glio) SF-295 | 66.0 |
| Brain (Amygdala) Pool | 4.9 |
| Brain (cerebellum) | 3.4 |
| Brain (fetal) | 6.4 |
| Brain (Hippocampus) Pool | 8.3 |
| Cerebral Cortex Pool | 6.0 |
| Brain (Substantia nigra) Pool | 5.4 |
| Brain (Thalamus) Pool | 7.5 |
| Brain (whole) | 5.8 |
| Spinal Cord Pool | 9.2 |
| Adrenal Gland | 15.9 |
| Pituitary gland Pool | 5.6 |
| Salivary Gland | 4.3 |
| Thyroid (female) | 28.1 |
| Pancreatic ca. CAPAN2 | 13.7 |
| Pancreas Pool | 22.8 |

TABLE ASG
Panel 5D

| | Rel. | Rel. | Rel. | Rel. | Rel. |
|---|---|---|---|---|---|
| | Exp. (%) | Exp. (%) | Exp. (%) | Exp. (%) | Exp. (%) |
| | Ag4695, | Ag4695, | Ag4700, | Ag4700, | Ag4705, |
| | Run | Run | Run | Run | Run |
| Tissue Name | 200923963 | 204244772 | 200923964 | 204244775 | 204245092 |
| 97457_Patient-02go_adipose | 21.5 | 23.3 | 77.9 | 94.6 | 24.1 |
| 97476_Patient-07sk_skeletal | 3.5 | 4.5 | 52.1 | 47.3 | 4.9 |
| muscle | | | | | |
| 97477_Patient-07ut_uterus | 8.7 | 7.1 | 25.9 | 18.0 | 6.6 |
| 97478_Patient-07pl_placenta | 66.9 | 69.7 | 100.0 | 100.0 | 69.7 |
| 97481_Patient-08sk_skeletal | 1.0 | 1.1 | 66.4 | 72.2 | 3.0 |
| muscle | | | | | |
| 97482_Patient-08ut_uterus | 1.6 | 8.0 | 10.9 | 7.2 | 7.4 |
| 97483_Patient-08pl_placenta | 30.1 | 30.6 | 39.2 | 54.0 | 26.6 |
| 97486_Patient-09sk_skeletal | 0.8 | 0.5 | 9.7 | 10.2 | 0.5 |
| muscle | | | | | |
| 97487_Patient-09ut_uterus | 4.9 | 3.1 | 21.2 | 14.5 | 4.3 |
| 97488_Patient-09pl_placenta | 35.6 | 54.7 | 77.9 | 65.1 | 47.3 |
| 97492_Patient-10ut_uterus | 8.8 | 10.7 | 34.2 | 25.5 | 8.3 |
| 97493_Patient-10pl_placenta | 100.0 | 100.0 | 79.0 | 97.9 | 100.0 |
| 97495_Patient-11go_adipose | 7.2 | 7.0 | 40.9 | 36.3 | 6.9 |
| 97496_Patient-11sk_skeletal | 0.9 | 0.8 | 12.3 | 6.7 | 1.7 |
| muscle | | | | | |
| 97497_Patient-11ut_uterus | 10.8 | 10.2 | 17.1 | 27.0 | 23.7 |
| 97498_Patient-11pl_placenta | 61.1 | 76.8 | 80.7 | 58.2 | 50.3 |
| 97500_Patient-12go_adipose | 10.2 | 0.0 | 70.2 | 57.8 | 12.7 |
| 97501_Patient-12sk_skeletal | 1.8 | 1.7 | 17.9 | 21.6 | 2.8 |
| muscle | | | | | |
| 97502_Patient-12ut_uterus | 14.5 | 13.2 | 35.8 | 51.1 | 18.4 |
| 97503_Patient-12pl_placenta | 72.2 | 70.7 | 72.7 | 52.5 | 68.8 |
| 94721_Donor 2 U - | 3.0 | 2.7 | 4.1 | 3.6 | 9.5 |
| A_Mesenchymal Stem Cells | | | | | |
| 94722_Donor 2 U - | 2.1 | 2.9 | 3.6 | 3.3 | 3.3 |
| B_Mesenchymal Stem Cells | | | | | |
| 94723_Donor 2 U - | 2.0 | 0.1 | 4.0 | 2.7 | 2.3 |
| C_Mesenchymal Stem Cells | | | | | |
| 94709_Donor 2 AM - A_adipose | 9.0 | 10.4 | 6.8 | 8.8 | 8.8 |
| 94710_Donor 2 AM - B_adipose | 6.5 | 5.5 | 5.8 | 2.9 | 5.2 |
| 94711_Donor 2 AM - C_adipose | 4.2 | 2.9 | 4.3 | 6.0 | 3.4 |
| 94712_Donor 2 AD - A_adipose | 7.2 | 8.0 | 16.2 | 11.7 | 7.6 |
| 94713_Donor 2 AD - B_adipose | 9.6 | 12.2 | 13.7 | 11.8 | 12.2 |
| 94714_Donor 2 AD - C_adipose | 8.8 | 9.7 | 9.3 | 7.0 | 12.9 |
| 94742_Donor 3 U - | 1.0 | 0.7 | 2.2 | 1.2 | 1.1 |
| A_Mesenchymal Stem Cells | | | | | |
| 94743_Donor 3 U - | 1.5 | 1.3 | 2.9 | 4.0 | 1.9 |
| B_Mesenchymal Stem Cells | | | | | |
| 94730_Donor 3 AM - A_adipose | 14.0 | 12.8 | 22.7 | 15.6 | 9.8 |
| 94731_Donor 3 AM - B_adipose | 7.2 | 29.1 | 7.0 | 10.8 | 6.8 |
| 94732_Donor 3 AM - C_adipose | 5.7 | 9.2 | 9.5 | 11.9 | 9.0 |
| 94733_Donor 3 AD - A_adipose | 17.2 | 20.3 | 17.0 | 20.6 | 15.3 |
| 94734_Donor 3 AD - B_adipose | 9.7 | 6.9 | 11.7 | 6.7 | 7.1 |
| 94735_Donor 3 AD - C_adipose | 11.1 | 11.9 | 19.2 | 13.8 | 10.3 |
| 77138_Liver_HepG2untreated | 27.5 | 27.5 | 34.2 | 39.2 | 23.3 |
| 73556_Heart_Cardiac stromal | 3.5 | 3.0 | 10.0 | 8.0 | 7.2 |
| cells (primary) | | | | | |
| 81735_Small Intestine | 13.3 | 12.1 | 49.0 | 48.0 | 15.5 |
| 72409_Kidney_Proximal | 5.8 | 5.1 | 15.0 | 8.4 | 5.6 |
| Convoluted Tubule | | | | | |
| 82685_Small | 17.9 | 19.5 | 60.3 | 44.8 | 28.1 |
| intestine_Duodenum | | | | | |
| 90650_Adrenal_Adrenocortical | 2.7 | 0.0 | 25.3 | 24.3 | 4.9 |
| adenoma | | | | | |
| 72410_Kidney_HRCE | 30.1 | 33.4 | 39.0 | 38.7 | 25.0 |
| 72411_Kidney_HRE | 28.5 | 23.2 | 40.9 | 50.0 | 22.4 |
| 73139_Uterus_Uterine smooth | 2.0 | 1.1 | 4.5 | 3.9 | 1.4 |
| muscle cells | | | | | |

General_screening_panel_v1.4 Summary: Ag4695/Ag4700/Ag4705 Three experiments using three probe-primer sets gave results that are in good agreement. This gene is expressed at moderate to high levels in all of the tissues on this panel, with highest expression in bladder and a lung cancer cell line (CTs=24-28). Interestingly, expression of this gene is higher in fetal lung and lung cancer cell lines when compared to adult lung. Expression of this gene is also upregulated in colon cancer cell lines when compared to normal colon. Therefore, therapeutic modulation of the activity of this gene or its protein product, through the use of small molecule drugs, protein therapeutics or antibodies, might be beneficial in the treatment of lung and colon cancer.

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

General_screening_panel_v1.5 Summary: Ag5877 Expression of this gene is highest in bladder (CT=23.6). This gene is expressed at moderate to high levels in all of the tissues on this panel, consistent with what is observed in Panel 1.4. Interestingly, expression of this gene is higher in fetal lung (CT=23.7)and a subset of lung cancer cell lines (CTs=24) when compared to adult lung (CT=28.2). Expression of this gene is also upregulated in colon cancer cell lines (CTs=24) when compared to normal colon (CT=27.2). Therefore, therapeutic modulation of the activity of this gene or its protein product, through the use of small molecule drugs, protein therapeutics or antibodies, might be beneficial in the treatment of lung and colon cancer. Please see Panel 1.4 for additional discussion of the potential relevance of this gene in human disease.

Panel SD Summary: Ag4695/Ag4705 Three experiments using two probe-primer sets gave results that are in good agreement. This gene is expressed at moderate to high levels in the majority of metabolic tissues on this panel, with highest expression in a placenta sample from a diabetic patient (CTs=23-28). Ag4700 Two experiment with same probe-primer sets are in excellent agreement. This gene shows widespread expression with highest expression of this gene in placenta of non-diabetic patient (CTs=30-30.7).

Spermine has been demonstrated to enhance insulin receptor binding in a dose dependent manner [Pedersen et al., Mol Cell Endocrinol., 1989 April;62(2): 161-6]. Thus, it was proposed that polyamines may act as intracellular or intercellular (autocrine) regulators to modulate insulin binding. It has also been shown that the insulin-like effects elicited by polyamines in fat cells (e.g. enhancement of glucose transport and inhibition of cAMP-mediated lipolysis) are dependent on H202 production (Livingston et al., J. Biol. Chem., Jan. 25, 1977;252(2):560-2). Inhibiting polyamine catabolism through an inhibitor of this rate-limiting enzyme may abolish the insulin-like antilipolytic effects of polyamines. Therefore, therapeutic inhibition of the activity of this gene using small molecule drugs may be of benefit in the treatment of obesity.

### Example D

Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

Results:

NOV 3b SNP Data

Two polymorphic variants of NOV3b have been identified and are shown in Table 3S.

TABLE 3S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13381488 | 314 | C | T | 65 | Ser | Ser |
| 13381501 | 803 | G | T | 228 | Val | Val |

NOV 5b SNP Data

One polymorphic variant of NOV5b has been identified and are shown in Table 5S.

TABLE 5S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13381503 | 3017 | G | A | 999 | Lys | Lys |

NOV 8a SNP Data

Four polymorphic variants of NOV8a have been identified and are shown in Table 8S.

TABLE 8S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| c34c- | 981 | G | C | 324 | Leu | Leu |
| cip1.113 | | | | | | |
| 13381270 | 1033 | A | G | 342 | Met | Val |
| 13381350 | 1042 | A | G | 345 | Ile | Val |
| 13376329 | 1222 | T | C | 405 | Ser | Pro |

NOV 9a SNP Data

Four polymorphic variants of NOV9a have been identified and are shown in Table 9S.

TABLE 9S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13381343 | 276 | C | T | 92 | Phe | Phe |
| 13381344 | 1045 | G | T | 349 | Ala | Ser |
| 13381348 | 1416 | C | T | 472 | Gly | Gly |
| 13381345 | 1802 | G | C | 601 | Gly | Ala |

NOV 10a SNP Data

One polymorphic variant of NOV10a has been identified and are shown in Table 10S.

TABLE 10S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13379513 | 1447 | C | T | 423 | Pro | Pro |

NOV 12a SNP Data

Two polymorphic variants of NOV12a have been identified and are shown in Table 12S.

TABLE 12S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13379505 | 139 | C | T | 15 | Pro | Ser |
| 13379506 | 221 | C | T | 42 | Ser | Phe |

NOV 13a SNP Data

Thirteen polymorphic variants of NOV13a have been identified and are shown in Table 13S.

TABLE 13S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13376183 | 75 | A | G | 2 | Gln | Gln |
| 13376184 | 182 | C | T | 38 | Ala | Val |
| 13376185 | 184 | G | A | 39 | Ala | Thr |
| 13376186 | 223 | A | G | 52 | Thr | Ala |
| 13376187 | 256 | C | T | 63 | Arg | Cys |
| 13376188 | 328 | A | G | 87 | Asn | Asp |
| 13376189 | 347 | C | T | 93 | Ala | Val |
| 13376190 | 373 | A | G | 102 | Thr | Ala |
| 13376191 | 1257 | C | T | 396 | Thr | Thr |
| 13376192 | 1342 | A | G | 425 | Ser | Gly |
| 13376193 | 1549 | G | A | 494 | Val | Met |
| 13376194 | 1581 | G | A | 504 | Thr | Thr |
| 13381349 | 1607 | A | G | 513 | Gln | Arg |

NOV 14a SNP Data

One polymorphic variant of NOV14a has been identified and are shown in Table 14S.

TABLE 14S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13376195 | 402 | T | C | 134 | Ala | Ala |

NOV 19 SNP Data

One polymorphic variant of NOV19 has been identified and are shown in Table 19S.

TABLE 19S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13381369 | 1380 | G | C | 460 | Ala | Ala |

NOV 20c SNP Data

One polymorphic variant of NOV20c has been identified and are shown in Table 20S.

TABLE 20S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13381370 | 281 | C | T | 94 | Thr | Met |

NOV 48a SNP Data

One polymorphic variant of NOV48a has been identified and are shown in Table 48S.

TABLE 48S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13381473 | 532 | C | G | 145 | Gln | Glu |

NOV 50a SNP Data

Two polymorphic variants of NOV50a have been identified and are shown in Table 50S.

TABLE 50S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13381514 | 744 | A | G | 242 | Ser | Gly |
| 13381513 | 1009 | T | C | 330 | Leu | Ser |

NOV 53b SNP Data

Six polymorphic variants of NOV53b have been identified and are shown in Table 53S.

TABLE 53S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13374617 | 437 | A | G | 143 | Asn | Ser |
| 13375310 | 664 | T | G | 219 | Phe | Val |
| 13375309 | 1150 | G | T | 381 | Ala | Ser |
| 13375308 | 1210 | G | T | 401 | Glu | End |
| 13375307 | 1770 | C | T | 587 | Asn | Asn |
| 13374615 | 2011 | A | G | 0 | | |

NOV 45b SNP Data

Two polymorphic variants of NOV54b have been identified and are shown in Table 54S.

TABLE 54S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13381471 | 472 | G | A | 145 | Pro | Pro |
| 13381470 | 1082 | A | G | 0 | | |

NOV 55a SNP Data

One polymorphic variant of NOV55a has been identified and are shown in Table 55S.

TABLE 55S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13375795 | 1070 | C | T | 236 | Arg | Trp |

NOV 56a SNP Data

Six polymorphic variant of NOV56a has been identified and are shown in Table 56S.

TABLE 56S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13375586 | 430 | T | C | 110 | Ser | Ser |
| 13375585 | 492 | A | G | 131 | Glu | Gly |
| 13375583 | 1756 | C | T | 552 | Asn | Asn |
| 13375582 | 2143 | T | A | 681 | Pro | Pro |
| 13377559 | 2550 | A | G | 817 | Lys | Arg |
| 13377776 | 2555 | C | T | 819 | Leu | Leu |

NOV 57b SNP Data

Two polymorphic variants of NOV57b have been identified and are shown in Table 57S.

TABLE 57S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13376786 | 1433 | G | A | 455 | Cys | Tyr |
| 13376785 | 1435 | A | G | 456 | Lys | Glu |

NOV 58a SNP Data

Two polymorphic variant of NOV58a has been identified and are shown in Table 58S.

TABLE 58S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13381335 | 499 | G | A | 145 | Glu | Glu |
| 13381336 | 1045 | C | T | 327 | Asn | Asn |

NOV 59b SNP Data

Three polymorphic variant of NOV59b has been identified and are shown in Table 59S.

TABLE 59S

| | Nucleotides | | | Amino Acids | | |
|---|---|---|---|---|---|---|
| | Base | | | Base | | |
| Variant | Position | Wild- | | Position | Wild- | |
| No. | of SNP | type | Variant | of SNP | type | Variant |
| 13379479 | 21 | T | C | 0 | | |
| 13381483 | 183 | C | T | 2 | Ala | Val |
| 13381482 | 520 | C | T | 114 | Ser | Ser |

### Example E

Method of Use

The present invention is partially based on the identification of biological macromolecules differentially modulated in a pathologic state, disease, or an abnormal condition or state, and/or based on novel associations of proteins and polypeptides and the nucleic acids that encode them, as identified in a yeast 2-hybrid screen using a cDNA library or one-by-one matrix reactions. Among the pathologies or diseases of present interest include metabolic diseases including those related to endocrinologic disorders, cancers, various tumors and neoplasias, inflammatory disorders, central nervous system disorders, and similar abnormal conditions or states. Important metabolic disorders with which the biological macromolecules are associated include obesity and diabetes mellitus, especially obesity and Type II diabetes. It is believed that obesity predisposes a subject to Type II diabetes. In very significant embodiments of the present invention, the biological macromolecules implicated in these pathologies and conditions are proteins and polypeptides, and in such cases the present invention is related as well to the nucleic acids that encode them. Methods that may be employed to identify relevant biological macromolecules include any procedures that detect differential expression of nucleic acids encoding proteins and polypeptides associated with the disorder, as well as procedures that detect the respective proteins and polypeptides themselves. Significant methods that have been employed by the present inventors, include GeneCalling® technology and SeqCalling™ technology, disclosed respectively, in U.S. Pat. No. 5,871,697, and in U.S. Ser. No. 09/417,386, filed Oct. 13, 1999, each of which is incorporated herein by reference in its entirety. GeneCalling® is also described in Shimkets, et al., Nature Biotechnology 17:198-803 (1999).

The invention provides polypeptides and nucleotides encoded thereby that have been identified as having novel associations with a disease or pathology, or an abnormal state or condition, in a mammal. Included in the invention are nucleic acid sequences and their encoded polypeptides. The sequences are collectively referred to as “obesity and/or diabetes nucleic acids” or “obesity and/or diabetes polynucleotides” and the corresponding encoded polypeptide is referred to as an “obesity and/or diabetes polypeptide” or “obesity and/or diabetes protein”. For example, an obesity and/or diabetes nucleic acid according to the invention is a nucleic acid including an obesity and/or diabetes nucleic acid, and an obesity and/or diabetes polypeptide according to the invention is a polypeptide that includes the amino acid sequence of an obesity and/or diabetes polypeptide. Unless indicated otherwise, “obesity and/or diabetes” is meant to refer to any of the sequences having novel associations disclosed herein.

The present invention identifies a set of proteins and polypeptides, including naturally occurring polypeptides, precursor forms or proproteins, or mature forms of the polypeptides or proteins, which are implicated as targets for therapeutic agents in the treatment of various diseases, pathologies, abnormal states and conditions. A target may be employed in any of a variety of screening methodologies in order to identify candidate therapeutic agents which interact with the target and in so doing exert a desired or favorable effect. The candidate therapeutic agent is identified by screening a large collection of substances or compounds in an important embodiment of the invention. Such a collection may comprise a combinatorial library of substances or compounds in which, in at least one subset of substances or compounds, the individual members are related to each other by simple structural variations based on a particular canonical or basic chemical structure. The variations may include, by way of nonlimiting example, changes in length or identity of a basic framework of bonded atoms; changes in number, composition and disposition of ringed structures, bridge structures, alicyclic rings, and aromatic rings; and changes in pendent or substituents atoms or groups that are bonded at particular positions to the basic framework of bonded atoms or to the ringed structures, the bridge structures, the alicyclic structures, or the aromatic structures.

The present invention discloses novel associations of proteins and polypeptides and the nucleic acids that encode them, as identified in a yeast 2-hybrid screen using a cDNA library or one-by-one matrix reactions. The proteins and related proteins that are similar to them are encoded by a cDNA and/or by genomic DNA and were identified in some cases by CuraGen Corporation.

In the current invention, protein interactions may include the interaction of a protein fragment with full-length protein, a protein fragment with another protein fragment, or full-length proteins with each other. The protein interactions disclosed in the present invention may also represent significant discoveries of functional importance to specific diseases or pathological conditions in which novel proteins are found to be components of known pathways, known proteins are found to be components of novel pathways, or novel proteins are found to be components of novel pathways.

A polypeptide or protein described herein, and that serves as a target in the screening procedure, includes the product of a naturally occurring polypeptide or precursor form or proprotein. The naturally occurring polypeptide, precursor or proprotein includes, e.g., the full-length gene product, encoded by the corresponding gene. The naturally occurring polypeptide also includes the polypeptide, precursor or proprotein encoded by an open reading frame described herein. A “mature” form of a polypeptide or protein arises as a result of one or more naturally occurring processing steps as they may occur within the cell, including a host cell. The processing steps occur as the gene product arises, e.g., via cleavage of the amino-terminal methionine residue encoded by the initiation codon of an open reading frame, or the proteolytic cleavage of a signal peptide or leader sequence. Thus, a mature form arising from a precursor polypeptide or protein that has residues 1 to N, where residue 1 is the N-terminal methionine, would have residues 2 through N remaining. Alternatively, a mature form arising from a precursor polypeptide or protein having residues 1 to N, in which an amino-terminal signal sequence from residue 1 to residue M is cleaved, includes the residues from residue M+1 to residue N remaining. A “mature” form of a polypeptide or protein may also arise from non-proteolytic post-translational modification. Such non-proteolytic processes include, e.g., glycosylation, myristylation or phosphorylation. In general, a mature polypeptide or protein may result from the operation of only one of these processes, or the combination of any of them.

As used herein, “identical” residues correspond to those residues in a comparison between two sequences where the equivalent nucleotide base or amino acid residue in an alignment of two sequences is the same residue. Residues are alternatively described as “similar” or “positive” when the comparisons between two sequences in an alignment show that residues in an equivalent position in a comparison are either the same amino acid or a conserved amino acid as defined below.

As used herein, a “chemical composition” relates to a composition including at least one compound that is either synthesized or extracted from a natural source. A chemical compound may be the product of a defined synthetic procedure. Such a synthesized compound is understood herein to have defined properties in terms of molecular formula, molecular structure relating the association of bonded atoms to each other, physical properties such as electropherographic or spectroscopic characterizations, and the like. A compound extracted from a natural source is advantageously analyzed by chemical and physical methods in order to provide a representation of its defined properties, including its molecular formula, molecular structure relating the association of bonded atoms to each other, physical properties such as electropherographic or spectroscopic characterizations, and the like.

As used herein, a “candidate therapeutic agent” is a chemical compound that includes at least one substance shown to bind to a target biopolymer. In important embodiments of the invention, the target biopolymer is a protein or polypeptide, a nucleic acid, a polysaccharide or proteoglycan, or a lipid such as a complex lipid. The method of identifying compounds that bind to the target effectively eliminates compounds with little or no binding affinity, thereby increasing the potential that the identified chemical compound may have beneficial therapeutic applications. In cases where the “candidate therapeutic agent” is a mixture of more than one chemical compound, subsequent screening procedures may be carried out to identify the particular substance in the mixture that is the binding compound, and that is to be identified as a candidate therapeutic agent.

As used herein, a “pharmaceutical agent” is provided by screening a candidate therapeutic agent using models for a disease state or pathology in order to identify a candidate exerting a desired or beneficial therapeutic effect with relation to the disease or pathology. Such a candidate that successfully provides such an effect is termed a pharmaceutical agent herein. Nonlimiting examples of model systems that may be used in such screens include particular cell lines, cultured cells, tissue preparations, whole tissues, organ preparations, intact organs, and nonhuman mammals. Screens employing at least one system, and preferably more than one system, may be employed in order to identify a pharmaceutical agent. Any pharmaceutical agent so identified may be pursued in further investigation using human subjects.

The following sections describe the study design(s) and the techniques used to identify these proteins, and any variants thereof, and to demonstrate its suitability as diagnostic markers, targets for an antibody therapeutic and targets for a small molecule drugs for Obesity and Diabetes.

Methods

1. RTQ-PCR (Real Time Quantitative Polymerase Chain Reaction) Technology:

The quantitative expression of various clones was assessed using microtiter plates containing RNA samples from a variety of normal and pathology-derived cells, cell lines and tissues using real time quantitative PCR (RTQ PCR). RTQ PCR was performed on a Perkin-Elmer Biosystems ABI PRISMS® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources), Panel 4 (containing cells and cell lines from normal cells and cells related to inflammatory conditions) and Panel CNSD.01 (containing samples from normal and diseased brains).

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

In the results for Panel 1, the following abbreviations are used:

ca.=carcinoma,

*=established from metastasis,

met=metastasis,

s cell var=small cell variant,

non-s=non-sm=non-small,

squam=squamous,

p1. eff=pl effusion=pleural effusion,

glio=glioma,

astro=astrocytoma, and

neuro=neuroblastoma.

Panel 1.4

The plates for panel 1.4 include 2 control wells (genomic DNA control and chemistry control) and 94 wells containing cDNA from various samples. The samples in panel 1.4 are broken into 2 classes; samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in panel 1.4 are widely available through the American Type Culture Collection, a repository for cultured cell lines. The normal tissues found on panel 1.4 are comprised of pools of samples from 2 to 5 different adult individuals derived from all major organ systems. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissue were ascertained to be free of disease and were purchased from various commercial sources such as Clontech (Palo Alto, Calf.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μ/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and +ve selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

Panel 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adiocyte differentiation was induced in donor progenitor cells obtained from Osirus (a division of Clonetics/BioWhittaker) in triplicate except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

| | | | |
|---|---|---|---|
| Donor 2 and 3: | U | Mesenchymal | Undifferentiated |
| | | Stem Cells | |
| Donor 2 and 3: | AM | Adipose | Adipose Midway |
| | | | Differentiated |
| Donor 2 and 3: | AD | Adipose | Adipose Differentiated |

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures.

All samples were processed at CuraGen to produce single stranded cDNA. RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01: Central Nervous System (CNS) Panel

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”Within each of these brains, the following regions are represented: cingulate gyrus, temporal pole, globus palladus, substantia nigra, Brodmann Area 4 (primary motor strip), Brodmann Area 7 (parietal cortex), Brodmann Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS panel the following abbreviations are used:

PSP: Progressive supranuclear palsy

Sub Nigra: Substantia nigra

Glob Palladus: Globus pallidus

Temp Pole: Temporal pole

Cing Gyr: Cingulate gyrus

BA: Brodmann Area

Method of Identifying the Differentially Expressed Gene and Gene Product:

The GeneCalling™ method makes a comparison between experimental samples in the amount of each cDNA fragment generated by digestion with a unique pair of restriction endonucleases, after linker-adaptor ligation, PCR amplification and chromatographic separation. Computer analysis is employed to assign potential identity to the gene fragment. Three methods are routinely used in the identification of a gene fragment found to have altered expression in models of or patients with obesity and/or diabetes.

Direct Sequencing: The differentially expressed gene fragment is isolated, cloned into a plasmid and sequenced. Afterwards the sequence information is used to design an oligonucleotide corresponding to either or both termini of the gene fragment. This oligonucleotide, when used in a competitive PCR reaction, will ablate the chromatographic band from which the sequence is derived.

Competitive PCR: In competitive PCR, the chromatographic peaks corresponding to the gene fragment of the gene of interest are ablated when a gene-specific primer (designed from the sequenced band or available databases) competes with primers in the linker-adaptors during the PCR amplification.

PCR with Perfect or Mismatched 3′ Nucleotides (Trapping): This method utilizes a competitive PCR approach using a degenerate set of primers that extend one or two nucleotides into the gene-specific region of the fragment beyond the flanking restriction sites. As in the competitive PCR approach, primers that lead to the ablation of the chromatographic band add additional sequence information. In conjunction with the size of the gene fragment and the 12 nucleotides of sequence derived from the restriction sites, this additional sequence data can uniquely define the gene after database analysis.

Antibodies

The invention further encompasses antibodies and antibody fragments, such as Fab, (Fab)₂ or single chain FV constructs, that bind immunospecifically to any of the proteins of the invention. Also encompassed within the invention are peptides and polypeptides comprising sequences having high binding affinity for any of the proteins of the invention, including such peptides and polypeptides that are fused to any carrier particle (or biologically expressed on the surface of a carrier) such as a bacteriophage particle.

Methods of Use of the Compositions of the Invention

The protein similarity information, expression pattern, cellular localization, and map location for the protein and nucleic acid disclosed herein suggest that this protein may have important structural and/or physiological functions characteristic of the Ornithine Decarboxylase 1 family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: Obesity and/or Diabetes.

These materials are further useful in the generation of antibodies that bind immunospecifically to the substances of the invention for use in diagnostic and/or therapeutic methods.

A. NOV10a—Human Ornithine Decarboxylase 1—CG124907-01

Discovery Process

The following sections describe the study design(s) and the techniques used to identify the ornithine decarboxylase 1-gene, encoded protein and any variants, thereof, as being suitable as diagnostic markers, targets for an antibody therapeutic and targets for a small molecule drugs for Obesity and Diabetes.

Studies: MB04. Mouse Obesity Model (Genetic)

Study Statements:

A large number of mouse strains have been identified that differ in body mass and composition. The AKR and NZB strains are obese, the SWR, C57L and C57BL/6 strains are of average weight whereas the SM/J and Cast/Ei strains are lean. Understanding the gene expression differences in the major metabolic tissues from these strains will elucidate the pathophysiologic basis for obesity. These specific strains of rat were chosen for differential gene expression analysis because quantitative trait loci (QTL) for body weight and related traits had been reported in published genetic studies. Tissues included whole brain, skeletal muscle, visceral adipose, and liver.

MB.08. Human Mesenchymal Stem Cell Differentiation

Bone marrow-derived human mesenchymal stem cells have the capacity to differentiate into muscle, adipose, cartilage and bone. Culture conditions have been established that permit the differentiation in vitro along the pathway to adipose, cartilage and bone. Understanding the gene expression changes that accompany these distinct differentiation processes would be of considerable biologic value. Regulation of adipocyte differentiation would have importance in the treatment of obesity, diabetes and hypertension. Human mesenchymal stem cells from 3 donors were obtained and differentiated in vitro according to published methods. RNA from samples of the undifferentiated, mid-way differentiated and fully differentiated cells was isolated for analysis of differential gene expression.

BP24.2. Diet Induced Obesity

The predominant cause for obesity in clinical populations is excess caloric intake. This so-called diet-induced obesity (DIO) is mimicked in animal models by feeding high fat diets of greater than 40% fat content. The DIO study was established to identify the gene expression changes contributing to the development and progression of diet-induced obesity. In addition, the study design seeks to identify the factors that lead to the ability of certain individuals to resist the effects of a high fat diet and thereby prevent obesity. The sample groups for the study had body weights +1 S.D., +4 S.D. and +7 S.D. of the chow-fed controls (below). In addition, the biochemical profile of the +7 S.D. mice revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues should reveal genes and pathways that can be used as therapeutic targets for obesity.

Ornithine Decarboxylase 1:

In multiple genecalling studies the enzyme spermidine/spermine acetyl transferase has been found to be dysregulated in various disease models. This enzyme is one of the rate-limiting enzymes in the production of polyamines spermidine and spermine. Previously, it was shown that oxidation of polyamines leads to generation of hydrogen peroxide, which has been shown to have antilipolytic effect of adipose and may therefore be involved in the progression of obesity. Ornithine decarboxylase catalyzes the first step in polyamine production, which is the conversion of ornithine to putrescine. The polyamine pathway can be detrimental for the obesity phenotype, since hydrogen peroxide produced during oxidation of polyamines in known to have anti-lipolytic, insulin-like effect on adipocytes. Therefore, inhibiting the production of polyamines and generation of H2O2 by inhibiting this first enzyme in the polyamine pathway may be beneficial in the treatment for obesity.

The Ornithine Decarboxylase 1 (ODC) is one of the key enzymes in polyamine biosynthesis. Preventing the accumulation of polyamines and their antilipolytic effects by inhibition of ODC at an earlier stage of obesity may inhibit progression of the obesity.

The following is a summary of the findings from the discovery studies, supplementary investigations and assays that also incorporates knowledge in the scientific literature for use of ornithine decarboxylase 1 as a diagnostic and/or target for small molecule drugs and antibody therapeutics. Taken in total, the data indicates that an inhibitor/antagonist of the human ornithine decarboxylase 1 would be beneficial in the treatment of obesity and/or diabetes.

SPECIES #1 Mouse (NZB vs SM/J):

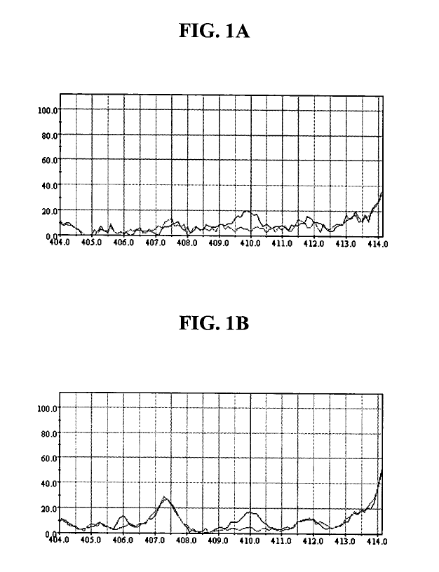

A gene fragment of the mouse spermine/spermidine N-acetyltransferase was initially found to be upregulated by 1.9 fold in the adipose of NZB mice relative to SM/J mice using CuraGen's GeneCalling™ method of differential gene expression. A differentially expressed mouse gene fragment migrating at approximately 411 nucleotides in length (FIG. 1a.—red vertical line) was definitively identified as a component of the mouse spermine/spermidine N-acetyltransferase cDNA in NZB and SM/J mouse strains. The method of competitive PCR was used for conformation of the gene assessment. The chromatographic peaks corresponding to the gene fragment of the mouse spermidine/spermine N-acetyltransferase are ablated when a gene-specific primer (see below) which competes with primers in the linker-adaptors during the PCR amplification. The peaks at 411 nt in length are ablated (green trace) in the sample from both the NZB and the SM/J mice. The altered expression in of these genes in the animal model support the role of Ornithine Decarboxylase 1 in the pathogenesis of obesity and/or diabetes.

SPECIES #1 Mouse (C57B1/6 Obese Euglycemic sd7 vs Obese sd1):

A gene fragment of the mouse spermine/spermidine N-acetyltransferase was initially found to be upregulated by 1.8 fold in the epididymal fat pad of the obese euglycemic sd7 mice relative to the obese sd1 mice using CuraGen's GeneCalling™ method of differential gene expression. A differentially expressed rat gene fragment migrating at approximately 178 nucleotides in length (FIG. 1a.—red vertical line) was definitively identified as a component of the mouse spermine/spermidine N-acetyltransferase cDNA in the Troglitazone treated and the untreated SHR control rats. The method of competitive PCR was used for conformation of the gene assessment. The chromatographic peaks corresponding to the gene fragment of the mouse spermidine/spermine N-acetyltransferase are ablated when a gene-specific primer (see below) which competes with primers in the linker-adaptors during the PCR amplification. The peaks at 178 nt in length are ablated (green trace) in the sample from both the C57B1/6 obese euglycemic sd7 and obese sd1 mice. The altered expression in of these genes in the animal model support the role of Ornithine Decarboxylase 1 in the pathogenesis of obesity and/or diabetes.

SPECIES #2 Human (Adipocyte Mid-Way vs Undifferentiated):

A gene fragment of the human spermine/spermidine N-acetyltransferase was initially found to be upregulated by 1.6 fold in the mid-way human adipocytes relative to the undifferentiated human adipocytes using CuraGen's GeneCalling™ method of differential gene expression. A differentially expressed human gene fragment migrating at approximately 194 nucleotides in length (FIG. 1a.—red vertical line) was definitively identified as a component of the human spermine/spermidine N-acetyltransferase cDNA in human mid-way differentiated and undifferentiated adipocytes. The method of competitive PCR was used for conformation of the gene assessment. The chromatographic peaks corresponding to the gene fragment of the human spermine/spermidine N-acetyltransferase are ablated when a gene-specific primer (see below) which competes with primers in the linker-adaptors during the PCR amplification. The peaks at 194 nt in length are ablated (green trace) in the sample from both the human mid-way differentiated and undifferentiated adipocytes. The altered expression of these genes in the human cellular model support the role of Ornithine Decarboxylase 1 in the pathogenesis of obesity and/or diabetes.

TABLE 1
Spermidine/spermine N-acetyltransferase Gene Sequence
identified in NZB vs SM/J mice

(Identified fragment from 206 to 616 in bold. band size: 411)

| | |
|---|---|
| 1 | GCTCCCGGGA AACGAATGAG GAACCACCTC CTCCTGCTGT TCAAGTACAC GGGCCTGGTG |
| 61 | CGCAAAGGGA AGAAAACCAA AAGACGAAAA TGGCTAAATT TAAGATCCGT CCAGCCACTG |
| 121 | CCTCTGACTG CAGTGACATC CTGCGACTGA TCAAGGAACT GGCTAAATAT GAATACATGG |
| 181 | AAGATCAAGT CATTTTAACT GAGAAAGATC TCCAAGAGGA TGGCTTTGGA GAACACCCCT |
| 241 | TCTACCACTG CCTGGTTGCA GAAGTGCCTA AAGAGCACTG GACCCCTGAA GGACATAGCA |
| 301 | TTGTTGGGTT CGCCATGTAC TATTTTACCT ATGACCCATG GATTGGCAAG TTGCTGTATC |
| 361 | TTGAAGACTT CTTCGTGATG AGTGATTACA GAGGCTTFGG TATAGGATCA GAAATTTTGA |
| 421 | AGAATCTAAG CCAGOTTGCC ATGAAGTGTC GCTGCAGCAG TATGCACTTC TTGGTAGCAG |
| 481 | AATGGAATGA ACCATCTATC AACTTCTACA AAAGAAGAGG TGCTTCGGAT CTGTCCAGTG |
| 541 | AAGAGGGATG GAGGCTCTTC AAGATTGACA AAGAGTACTT GCTAAAAATG GCAGCAGAGG |
| 601 | AGTGAGGCGT GCCCGTGTAG ACAATGACAA CCTCCATTGT GCTTTAGAAT AATTCTCAGC |
| 661 | TTCCCTTGCT TTCTATCTTG TGTGTAGTGA AATAATAGAC CGAGCACCCA TTCCAAAGCT |
| 721 | TTATTACCAG TGACGTTCTT GCATGTTTGA AATTCGCTCT CITTAAAGTG GCAGTCATGT |
| 781 | ATCTGGTTTG GAGCCAGAAT TCTTGAACAT CTTTTGATGA ACAACAACGT GGTATGATCT |
| 841 | TACTATATAA GAAAAACAAA ACTTCATTCT TGTGAGTCAT TTAAATGTGT ACAATGTACA |
| 901 | CACTGGTACT TAGAGTTTCT GTTTTCATTC TTTTTTTTTA AATAAACTCC CTCTTTGATT |
| 961 | T |

TABLE 2
Spermidine/spermine N-acetyltransferase Gene Sequence
identified in C57B1/6 obese euglycemic sd7 vs obese sd1

(Identified fragment from 716 to 893 in bold. band size: 178)

| | |
|---|---|
| 235 | ACCCCTTCTA CCACTGCCTC GTTCCACAAG TGCCTAAAGA GCACTGGACC CCTGAAGGAC |
| 295 | ATACCATTGT TGGGTTCGCC ATGTACTATT TTACCTATGA CCCATCGATT GGCAAGTTGC |
| 355 | TGTATCTTGA ACACTTCTTC CTGATGAGTG ATTACACACG CTTTCGTATA CGATCACAAA |
| 415 | TTTTGAAGAA TCTAAGCCAG GTTGCCATGA AGTGTCGCTG CACCACTATG CACTTCTTGG |
| 475 | TAGCAGAATG GAATGAACCA TCTATCAACT TCTACAAAAG AAGAGGTGCT TCGGATCTGT |
| 535 | CCAGTGAAGA GGGATGGAGG CTCTTCAAGA TTGACAAAGA GTACTTGGTA AAAATGGCAG |
| 595 | CAGACCACTC AGCCCTCCCC GTCTAGACAA TGACAACCTC CATTGTGCTT TAGAATAATT |
| 655 | CTCAGCTTCC CTTCCTTTCT ATCTTCTCTG TAGTCAAATA ATACACCGAG CACCCATTCC |
| 715 | AAAGCTTTAT TACCAGTGAC GTTGTTGCAT GTTTGAAATT CGGTCTGTTT AAAGTGGCAG |
| 775 | TCATGTATGT GGTTTGGAGG CAGAATTCTT GAACATCTTT TGATGAAGAA CAAGGTGGTA |
| 835 | TGATCTTACT ATATAAGAAA AACAAAACTT CATTCTTGTG AGTCATTTAA ATGTGTACAA |
| 895 | TGTACACACT GCTACTTACA GTTTCTGTTT TGATTCCTTT TTTTTAAATA AACTCGCTCT |
| 955 | TTGATTT |

TABLE 3
Spennidine/spermine N-acetyltransferase Gene Sequence
identified in human adipocyte mid-way versus undifferentiated

(Identified fragment from 162 to 355 in bold. band size: 149).

| | |
|---|---|
| 1 | CTGGTGTTTA TCCGTCACTC GCCGAGGTTC CTTCGGTCAT GGTGCCAGCC TGACTGAGAA |
| 61 | GAGGACGCTC CCGGGAGACG AATGAGGAAC CACCTCCTCC TACTGTTCAA CTACAGGGGC |
| 121 | CTGGTCCGCA AAGGGAAGAA AAGCAAAAGA CGAAAATGGC TAAATTCGTG ATGCGCGCAG |
| 181 | CCACTGCCGC CGACTGCAGT GACATACTOC GGCTGATCAA GGAGCTGGCT AAATATGAAT |
| 241 | ACATGGAAGA ACAAGTAATC TTAACTGAAA AAGATCTGCT AGAAGATGGT TTTGGAGAGC |
| 301 | ACCCCTTTTA CCACTGCCTG GTTGCAGAAG TGCCGAAAGA GCACTGGACT CCGGAAGCTT |
| 361 | ACAGTCTCTA GCTTCGCCAT GTACATGGCC CTTCCGTGTA CATGGATGGG CGGGGAGGTA |
| 421 | ACTAAAAGAT CCTTTACACA ATAAAGTAGA TGATCATGAT AAATGAGGAC ACAGCATTGT |
| 481 | TGGTTTTGCC ATGTACTATT TTACCTATGA CCCCTCCATT GCCAAGTTAT TGTATCTTGA |
| 541 | GGACTTCTTC GTGATGAGTG ATTATAGAGG CTTTGGCATA GGATCAGAAA TTCTCAAGAA |
| 601 | TCTAAGCCAG GTTGCAATGA GGTGTCGCTG CAGCAGCATG CACTTCTTGG TACCAGAATG |
| 661 | GAATGAACCA TCCATCAACT TCTATAAAAG AAGAGGTGCT TCTGATCTGT CCAGTCAAGA |
| 721 | GGGTTGGAGA CTGTTCAAGA TCGACAAGGA GTACTTGCTA AAAATGGCAA CAGAGGACTG |
| 781 | ACGAGTGCTG CTGTAGATGA CAACCTCCAT TCTATTTTAG AATAAATTCC CAACT |

TABLE 4
Human Ornithine Decarboxylase 1 gene
and protein sequence.

>CG124907-01 1958 nt
GCAGGCCAGCCCCATGGGGAAGCCCAGACGCCGGNGCCTCGGCGCTCTGA
GATTGTCACTGCTCTTCCAAGGGCACACGCAGAGGGATTTGGAATTCCTG
GAGAGTTCCCTTTGTGAGAAGCTCGAAATATTTCTTTCAATTCCATCTCT
TAGTTTTCCATAGGAACATCAAGAAATCATGAACAACTTTGGTAATGAAG
AGGTTGACTGCCACTTCCTCGATGAAGGTTTTACTCCCAAGCACATTCTG
GACCAGAAAATTAATGAAGTTTCTTCTTCTGATGATAAGGATGCCTTCTA
TGTGGCAGACCTGGGAGACATTCTAAAGAAACATCTGACCTGGTTAAAAG
CTCTCCCTCGTGTCACCCCCTPTTATGCAGTCAAATCTAATGATAGCAAA
GCCATCGTGAACACCCTTGCTGCTACCGGGACAGGATTTGACTCTGCTAG
CAAGACTGAAATACAGTTGGTGCAGAGTCTGGGGGTGCCTCCAGAGAGGA
TTATCTATGCAAATCCTTGTAAACAAGTATCTCAAATTAAGTATGCTGCT
AATAATCGAGTCCAGATCATGACTTTTGATAGTCAAGTTGAGTTCATCAA
AGTTGCCAGAGCACATCCCAAAGCAAAGTTGGTTTTCCGGATTGCCACTG
ATCATTCCAAAGCAGTCTGTCGTCTCACTGTGAAATTCGGTGCCACGCTC
ACAACCAGCAGGCTCCTTTTGGAACCGGCCAAAGAGCTAAATATCGATGT
TGTTGGTGTCAGCTTCCATGTAGGAAGCCGCTGTACCGATCCTGACACCT
TCGTGCAGGCAATCTCTGATGCCCGCTGTGTTTTTGACATGGGGCTCTAC
GTTGGTTTCAGCATGTATCTGCTTGATATTGGCGGTCGCTTTCCTGGATC
TGAGGATGTGAAACTTAAATTTGAAGAGATCACCGGCGTAATCAACCCAG
CGTTGGACAAATACTTTCCGTCAGACTCTGGAGTGAGAATCATAGCTGAG
CCCGGCAGATACTATGTTGCATCAGCTTTCACGCTTGCAGTTAATATCAT
TGCCAAGAAAATTGTATTAAAGGAACAGACGGGCTCTGATGACGAAGATG
AGTCGAGTGAGCACACCTTTATGTATTATGTGAATGATGGCGTCTATGGA
TCATTTAATTGCATACTCTATGACCACGCACATGTAAACCCCCTTCTGCA
AAAGAGACCTAAACCAGATGAGAAGTATTATTCATCCAGCATATGGGGAC
CAACATGTGATGGCCTCGATCCGATTGTTGAGCGCTGTGACGTGCCTGAA
ATGCATCTCGCTCATTCGATGCTCTTTGAAAACATCCCCCCTTACACTGT
TGCTGCTGCCTCTACGTTCAATGGCTTCCAGAGGCCGACGATCTACTATG
TGATGTCAGGGCCTGCGTGGCAACTCATGCAGCAATTCCAGAACCCCGAC
TTCCCACCCGAAGTAGAGGAACAGGATGCCAGCACCCTGCCTGTGTCTTG
TGCCTGGGAGAGTGGGATGAAACGCCACAGAGCAGCCTGTGCTTCGGCTA
GTATTAATGTGTAGATAGCACTCTGGTAGCTCTTAACTGCAAGTTTAGCT
TGAATTAAGGGATTTCGGGGGACCATGTAACTTAATTACTGCTAGTTTTG
AAATGTCTTTGTAAGAGTAGGGTCGCCATGATGCAGCCATATGGAAGACT
ACCATATGGGTCACACTTATCTGTGTTCCTATGGAAACTATTTGAATATT
TGTTTTATATGGATTTTTATTCACTCTTCAGACACCCTACTCAACAGTCC
CCCTCAGGTGCTGAACAAGCATTPGTAGCTTGTACAATGGCAGAATGGGC
CAAAAGCTTAGTGTTGTGACCTGTTTTTAAAATAAACTATCTTCAAATAA
ATAAAAAAAAAAAAGGGGGGCCGCCCTAGGGGTTCCCAAGTTTACGTACG
CTCCATCG

TABLE 5
Human Ornithine Decarboxylase 1 protein sequence>

ORF Start: 179 ORF Stop: 1562 Frame: 2
Human Ornithine Decarboxylase 1 Protein Sequence:
>CG124907-O1-prot 461 aa
MNNFGNEEFDCHFLDEGFTAKDILDQKINEVSSSDDKDAFYVADLGDILK
KHLRWLKALPRVTPFYAVKCNDSKAIVKTLAATGTGFDCASKTEIQLVQS
LGVPPERIIYANPCKQVSQIKYAANNGVQMMTFDSEVELMKVARAHPKAK
LVLRIATDDSKAVCRLSVKFGATLRTSRLLLERAKELNIDVVGVSFEVGS
GCTDPETEVQAISDARCVEDMGAEVGFSMYLLDIGGGFPGSEDVKLKFEE
TTGVINFALDKYFPSDSGVRIIAEPCRYYVASAFTLAVNIIAKKIVLKEQ
TGSDDEDESSEQTFMYYVNDGVYGSFNCILYDEAHVKPLLQKRPKPDEKY
YSSSIWGPTCDGLDRIVERCDLPEMHVGDWMLFENNGAYTVAAASTFNGF
QRPTIYYVMSGPAWQLMQQFQNPDFPPEVEEQDASTLPVSCAWESGMKRH
RAACASASINV

[image]

[image]

In addition to the human version of the Ornithine Decarboxylase 1 identified as being differentially expressed in the experimental study, other variants have been identified by direct sequencing of cDNAs derived from many different human tissues and from sequences in public databases. No splice-form variants have been identified at CuraGen whereas several amino acid-changing cSNPs were identified. These are found below. The preferred variant of all those identified, to be used for screening purposes, is CG124907-01.

TABLE 7
Variants of human Ornithine Decarboxylase 1 obtained
from direct cloning and/or public databases.

| DNA | | | | | |
|---|---|---|---|---|---|
| Position | Strand | Alleles | AA Position | AA Change | public SNP# |
| 1447 | Minus | C:T | 423 | Pro => Pro | |

[image]

FIGS. 1A and 1B show differential regulation of spermidine/spermine N-acetyltransferase in the expressed gene fragment in Discovery Study MB.04 of NZB vs SM/J mice. The abscissa on each graph is measured in length of nucleotides, and the ordinate is measured in signal response.

[image]

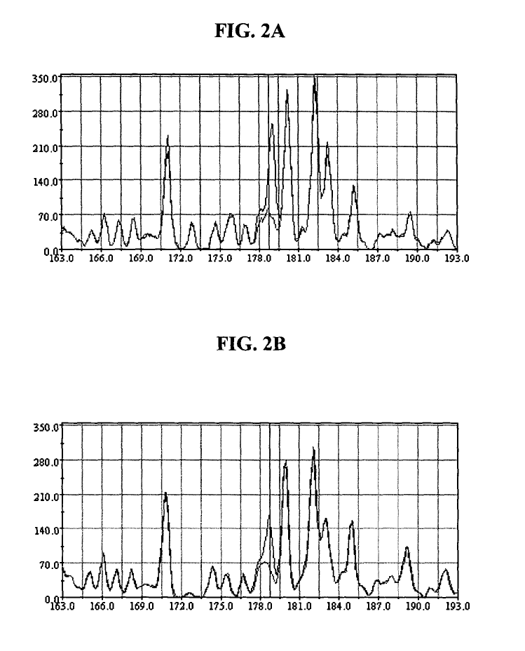

FIGS. 2A and 2B show differential regulation of spermidine/spermine N-acetyltransferase in the expressed gene fragment in Discovery Study MB.04 of NZB vs SM/J mice. The abscissa on each graph is measured in length of nucleotides, and the ordinate is measured in signal response.

[image]

[image]

[image]

Species #1 Mouse Strains NZB, SM/J, C56B1/6

Species # 2 Human

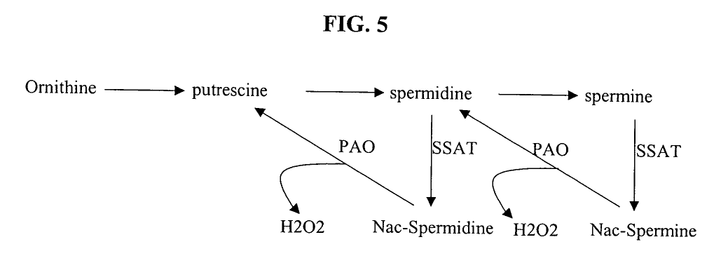

FIG. 5 summarize the biochemistry surrounding the human Ornithine Decarboxylase 1 and potential assays that may be used to screen for antibody therapeutics or small molecule drugs to treat obesity and/or diabetes. Cell lines expressing the Ornithine Decarboxylase 1 can be obtained from the RTQ-PCR results shown above. These and other Ornithine Decarboxylase 1 expressing cell lines could be used for screening purposes. In the schematic, the biochemistry of “PAO” is that it catalyses oxidation of the secondary amino group of spermine, spermidine and their acetyl derivatives; FAD is the cofactor implicated; and the schematic is shown in monomeric units.

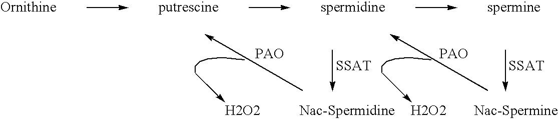

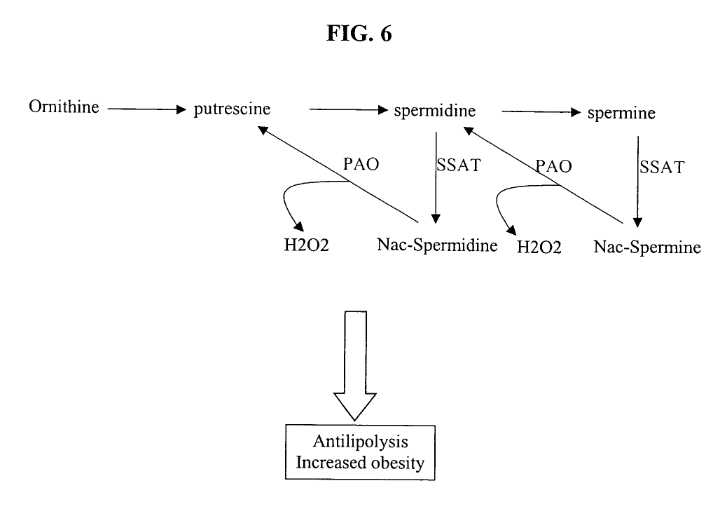

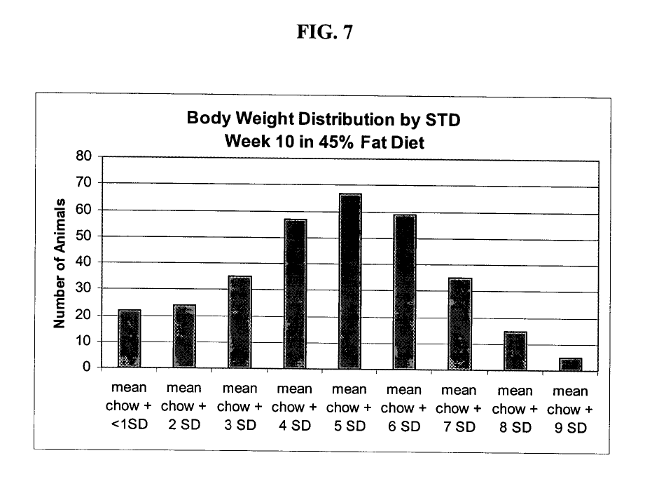

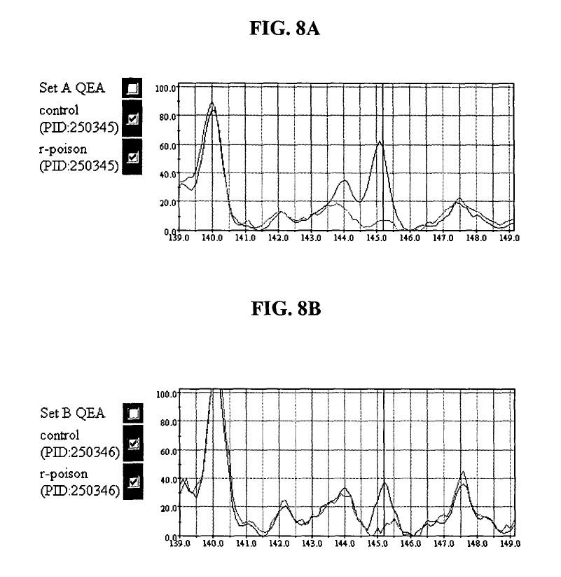

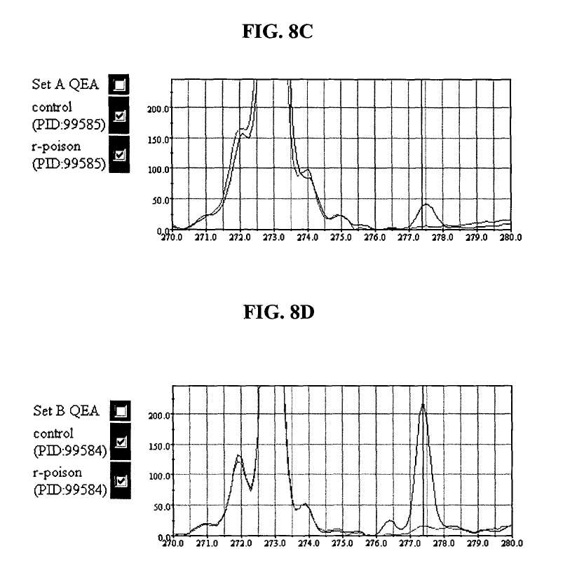

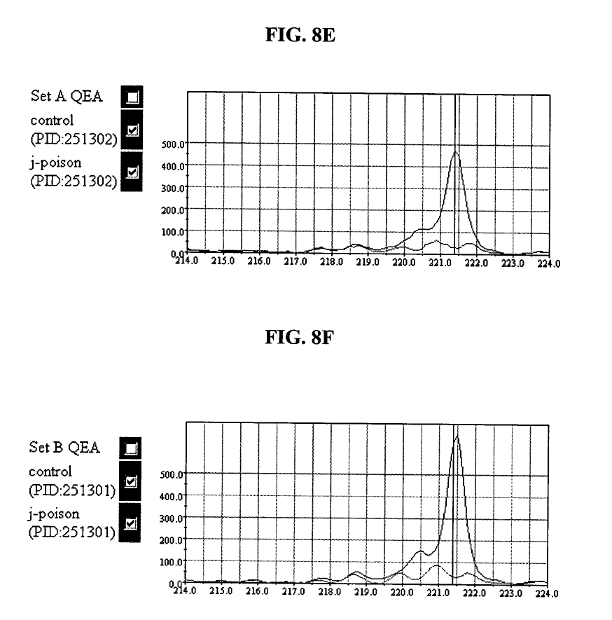

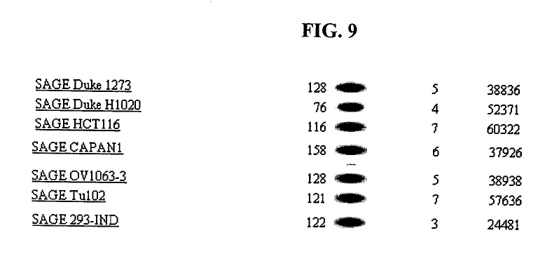

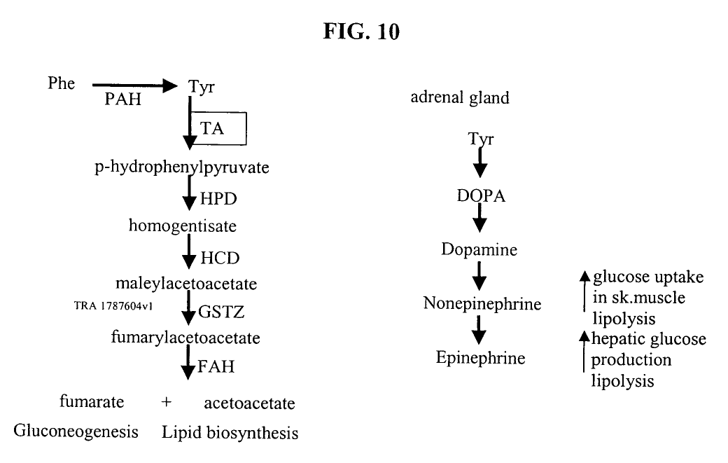

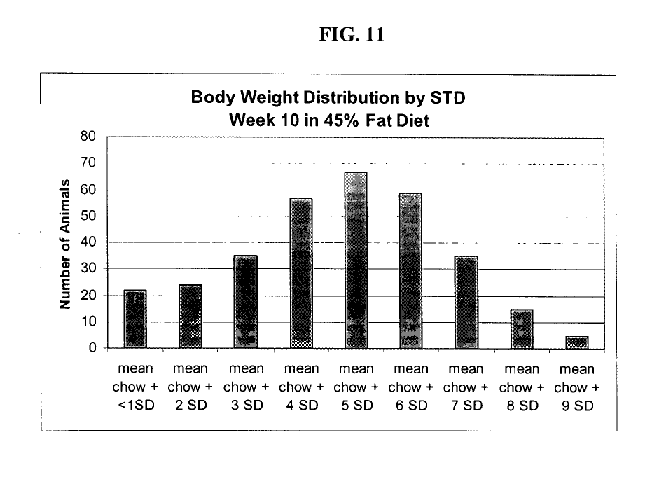

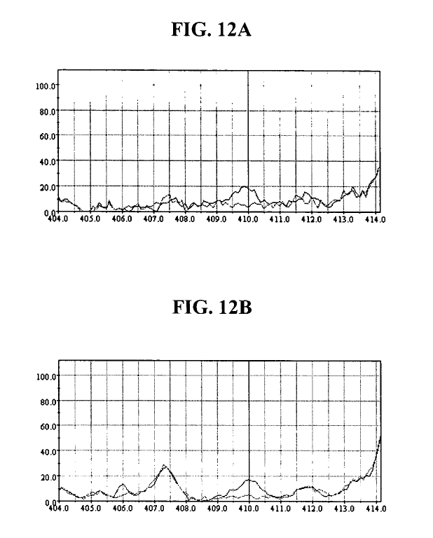

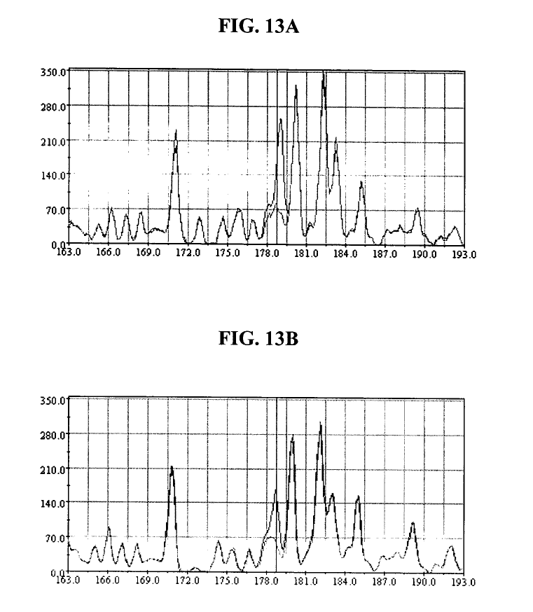

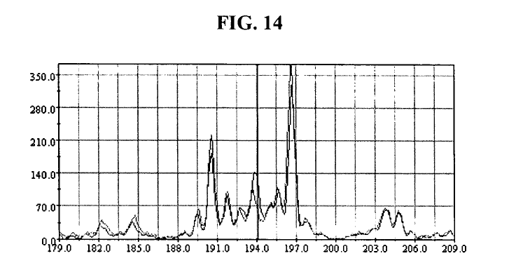

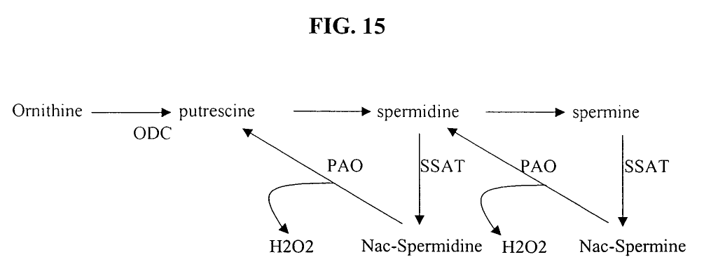

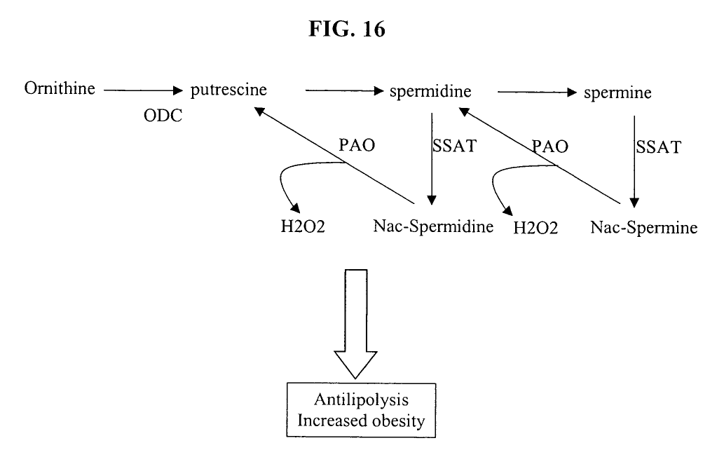

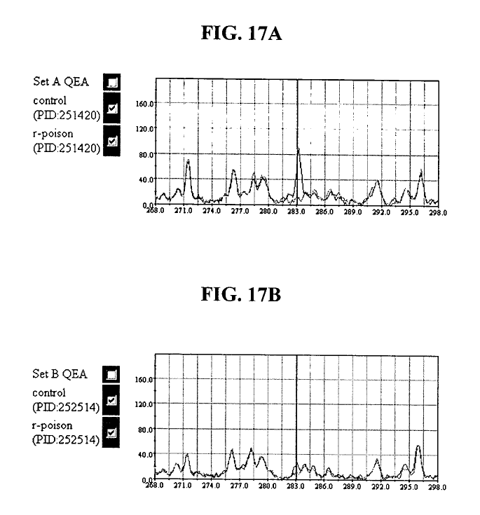

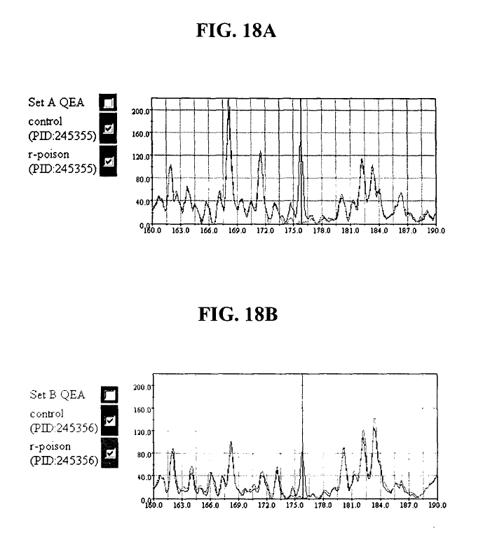

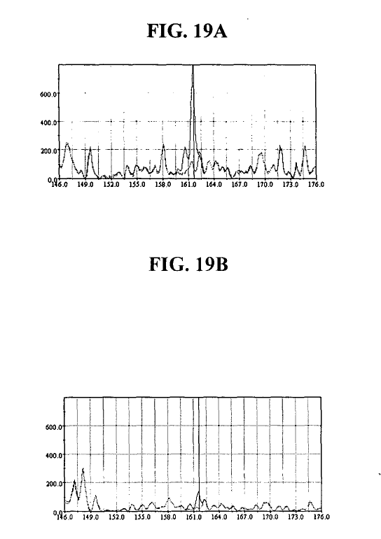

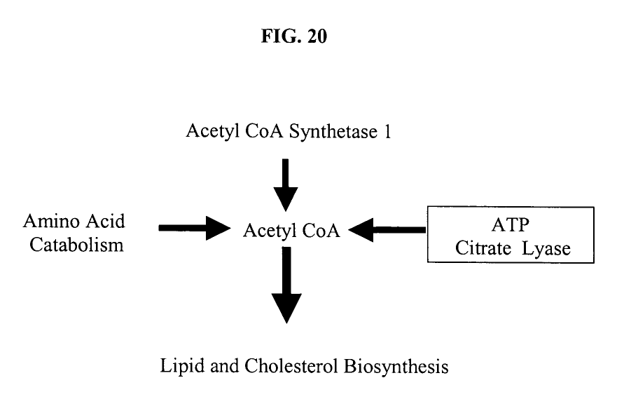

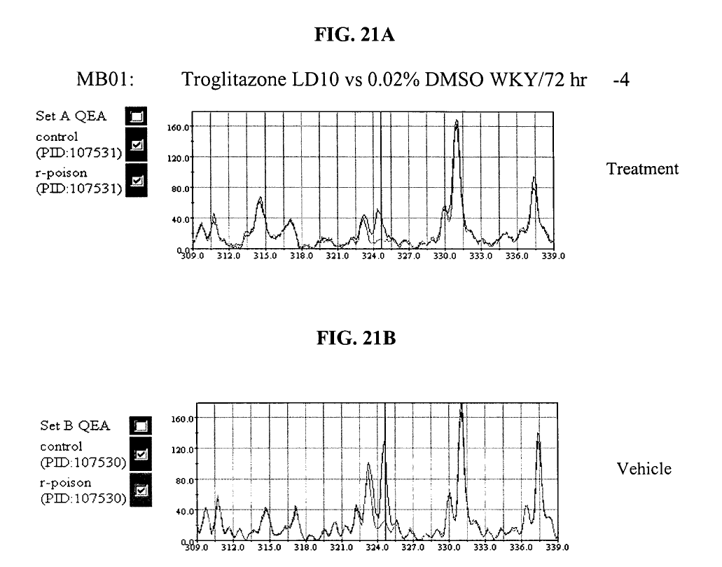

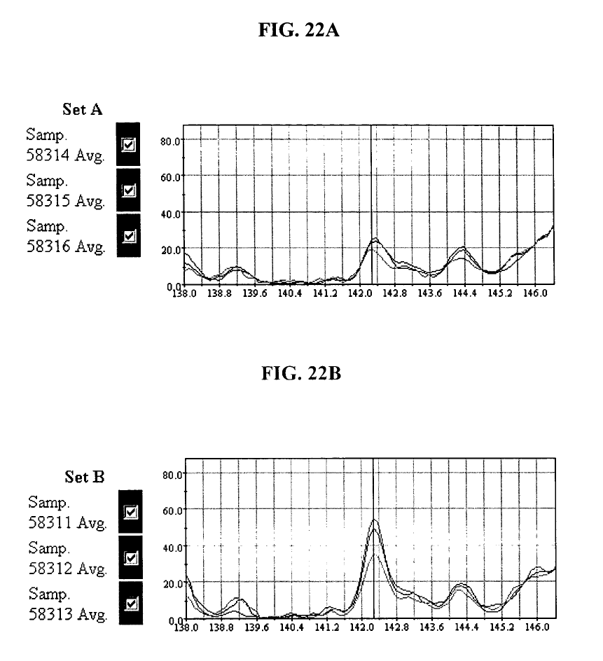

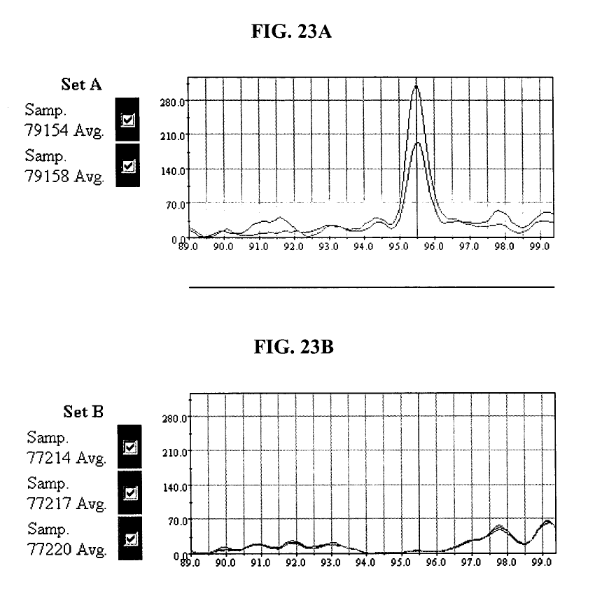

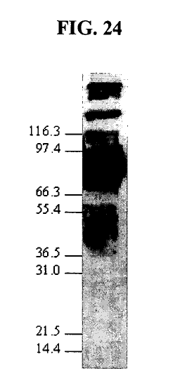

FIG. 6 suggests how alterations in expression of the human ornithine decarboxylase 1 and associated gene products function in the etiology and pathogenesis of obesity and/or diabetes. The scheme incorporates the unique findings of these discovery studies in conjunction with what has been reported in the literature. The outcome of inhibiting the action of the human ornithine decarboxylase 1 would be a way to increase lypolysis by inhibiting anti-lypolytic effects of hydrogen peroxide.

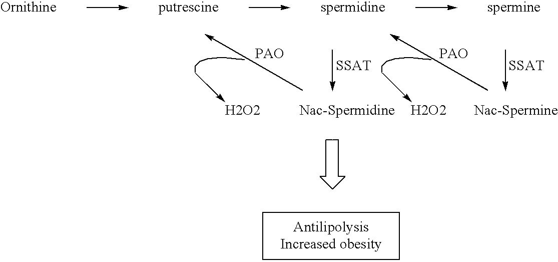

Ornithine decarboxylase catalyzes the first step in polyamine production, the conversion of ornithine to putrescine. Inhibiting the production of polyamines and H2O2 by inhibiting this first enzyme in the pathway will eliminate the lipolytic effects of H2O2 and therefore may be beneficial in the treatment for obesity.

The following is a summary of the findings from the discovery studies, supplementary investigations and assays that also incorporates knowledge in the scientific literature. Taken in total, the data indicates that an inhibitor/antagonist of the human Ornithine Decarboxylase 1 would be beneficial in the treatment of obesity and/or diabetes.

In multiple genecalling studies the enzyme spermidine/spermine acetyl transferase was found to be dysregulated in various disease models. This enzyme is one of the rate-limiting enzymes in the production of polyamines spermidine and spermine. Previously, it was shown that oxidation of polyamines leads to generation of hydrogen peroxide, which has been shown to have antilipolytic effect of adipose and may therefore be involved in the progression of obesity. Ornithine decarboxylase catalyzes the first step in polyamine production, which is the conversion of ornithine to putrescine. The polyamine pathway can be detrimental for the obesity phenotype, since hydrogen peroxide produced during oxidation of polyamines in known to have anti-lipolytic, insulin-like effect on adipocytes. Therefore, inhibiting the production of polyamines and generation of H2O2 by inhibiting this first enzyme in the polyamine pathway may be beneficial in the treatment for obesity.

B. NOV12A—Tyrosine Aminotransferase—CG135823-01

The present invention discloses novel associations of proteins and polypeptides and the nucleic acids that encode them with various diseases or pathologies. The proteins and related proteins that are similar to them, are encoded by a cDNA and/or by genomic DNA. The proteins, polypeptides and their cognate nucleic acids were identified by CuraGen Corporation in certain cases. The Tyrosine Aminotransferase-encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

TABLE 1
SPECIES #1, Rat Tyrosine Aminotransferase Gene Fragment
used for competitive PCR

(fragment from 845 to 989 in bold. band size: 145)

| | |
|---|---|
| 364 | CCTACAGACC CTGAAGTTAC CCAAGCCATG AAAGATCCMC TGGACTCGGG GAAGTACAAT |
| 424 | GGCTATGCCC CGTCCATCGG CTACCTATCC AGTCGGGAGG AGGTCGCTTC TTACTACCAC |
| 484 | TGTCATGAGG CTCCTCTCGA AGCTAAGGAT GTCATTCTGA CAAGCCGCTG CAGTCAGGCC |
| 544 | ATTGAGCTAT GTCTAGCTCT GTTGGCCAAT CCTGGACAAA ACATCCTCAT TCCAACGCCC |
| 604 | CGGTTTTCCC TCTATAGGAC TTTGGCTGAG TCTATGGGAA TTGAGGTCAA GCTCTACAAT |
| 664 | CTCCTCCCCC AGAAGTCTTG GGAAATTGAC CTAAAACAAC TGGAATCTCT GATCGATGAA |
| 724 | AAAACAGCGT GTCTTGTTGT CAACAACCCA TCCAATCCCT GTCGCTCCGT GTTCAGTAAG |
| 784 | CCACACCTTC AGAAGATTTT GGCAGTGGCT GAAAGGCAGT GTCTCCCCAT CTTAG6TGAC |
| 844 | GAGATCTATG GTGACATGGT GTTTTCAGAT TGCAAATACG AACCACTGGC CAACCTCAGC |
| 904 | ACCAATGTTC CCATCCTGTC CTGTGGTGGG CTCGCCAAGC CCTGGCTGGT CCTTGGCTGG |
| 964 | AGGTTGGGCT GGATCCTCAT TCATGATCGA AGAGACATTT TTGGCAATGA GATTCGAGAC |
| 1024 | GGGCTCCTGA AACTGAGTCA GCGGATCCTG GGACCATGCA CCATAGTCCA GGGTGCTCTG |
| 1084 | AAGAGCATCC TTCAGCGAAC CCCTCAGGAG TTCTATCACG ACACGTTAAG CTTCCTCAAG |
| 1144 | TCCAATGCGG ACCTCTGCTA TGGGGCACTG GCTGCCATCC CTGGACTCCA GCCGGTCCGC |
| 1204 | CCTTCTGGAG CCATGTACCT TATGGTGGGA ATTGAGATGG AGCATTTCCC GGAATTCGAG |
| 1264 | AACGACGTGG AGTTCACAGA GCCGTTGATT GCGGAGCACG CTGTCCACTG TCTCCCAGCA |
| 1324 | ACGTGCTTCG AGTACCCAAA TTTCTTCCGA GTGGTCATCA CACTCCCCCA GGTCATCATG |
| 1384 | CTGGAGGCTT GTAGCCGGAT CCAGGAGTTC TGTGAACAGC AGTACCACTG TGCTGAAGGC |
| 1444 | AGCCAGGAGG AGTGTGACAA ATAAGC |

(gene length is 2364, only region from 364 to 1469 shown)

TABLE 2
SPECIES #2, Rat Tyrosine Aminotransferase Gene Fragment
used for competitive PCR

(fragment from 1 to 277 in bold. band size: 277).

| | |
|---|---|
| 1 | TCATGATCCA AGAGACGTTT TTGGCAATGA GATTCGAGAC GGGCTGGTGA AACTGAGTCA |
| 61 | GCCGATCCTG GGACCATGCA CCATAGTCCA GGGTCCTCTG AAGAGCATCC TTCAGCGAAC |
| 121 | CCCTCACGAG TTCTATCACG ACACGTTAAG CTTCCTCAAG TCCAATGCGG ACCTCTGCTA |
| 181 | TCGGGCACTG GCTGCCATCC CTGGACTCCA GCCGGTCCGC CCTTCTGGAG CCATGTACCT |
| 241 | TATGGTGGGA ATTGAGATGG AGCATTTCCC GGAATTC |

(gene length is 277, only region from 1 to 277 shown)

TABLE 3
SPECIES #3, Mouse Tyrosine Aminotransferase Gene Fragment
used for competitive PCR

(fragment from 57 to 275 in bold. band size: 220)

| | |
|---|---|
| 1 | CCTTCAGAAG ATTTTGGCAG TGGCTGAAAG GCAATCCGTC CCCATCTTAG CCGATGAGAT |
| 61 | CTATGGTGAC ATGGTGTTET CAGATTGCAA ATATGAACCA ATGGCCACCC TCAGCACCAA |
| 121 | TGTCCCCATC CTGTCCTGTG GTGGGCTCGC CAAGCGCTGQ CTGGTTCCTG GCTGGAGGCT |
| 181 | GGGCTGGATC CTTATCCATG ATCGAAGAGA CATTTTTGGC AATGAGATTC GGGACGGGCT |
| 241 | GGTGAAGCTG AGTCAGCGGA TCCTGGGCCC GTGCACCATC GTCCAAGGTG CCCTGAAGAG |
| 301 | CATCCTTCAG CCCACCCCTC AGGAGTTCTA CCAGGACACT TTAACCTTCC TTAAGTCCAA |
| 361 | TGCGGACCTC TCCTATGGGG CGTTGTCTGC AATTCCTGGA CTCCAGCCAG TCCGCCCATC |
| 421 | TGGAGCCATG TACCTTATGG TGGGAATTGA GATGGAGCAC TTCCCAGAAT TTGAGAATGA |
| 481 | CGTGGAATTC ACAGAGCGGT TAATTGCGGC AGNNTCTGTC GNACTGCTCC AGCACGTGCT |
| 541 | TCGACTACCA ATTTCTTCCG CCTGTCATAC AGTCCCCGAG TGATGATCCT G |

(gene length is 592, only region from 1 to 592 shown)

TABLE 4
Human Tyrosine Aminotransferase gene
and protein sequence.

>CG135823-01 2754 nt
ATTGCCCCTGTAACCTGTCAAAGAAGAGCTAAGCGAGCTTTCGCGGTTGG
CTTCTTGGAGGCTGCTTTCTCCTTTACTTGCAAGGCTTCGCTAGTGATGG
ACCCATACATGATTCAGATGAGCAGCAAAGGCAACCTCCCCTCAATTCTG
GACGTGCATGTCAACCTTGGTGGGACAAGCTCTGTGCCGGGAAAAATGAA
AGGCAGAAAGGCCAGGTGGTCTGTGAGGCCCTCAGACATGCCCAACAAAA
GTTTCAACCCCATCCGAGCCATTGTGGACAACATGAAGGTGAAACCAAAT
CCAAACAAAACCATGATTTCCCTGTCCATTGGGGACCCTACTGTCTTTGG
AAACCTGCCTACAGACCCTGAAGTTACCCAGGCAATGAAAGATGCCCTGG
ACTCGCGCAAATATAATGGCTATGCCCCATCCATCGGCTTCCTATCCAGT
CGGGAGGAGATTCCTTCTTATTACCACTCTCCTGAGGCACCCCTAGAAGC
TAAGGACGTCATTCTGACAAGTCGCTGCAGCCAAGCTATTCACCTTTGTT
TAGCTGTGTTGGCCAACCCAGGGCAGAACATCCTGGTTCCAAGACCTGGT
TTCTCTCTCTACAAGACTCTGGCTGAGTCTATGGGAATTGAGGTCAAACT
CTACAATTTGTTGCCAGAGAAATCTTGGGAAATTGACCTGAAACAACTCG
AATATCTAATTGATGAAAAGACAGCTTGTCTCATTGTCAATAATCCATCA
AACCCCTGTGGGTCAGTGTTCAGCAAACGTCATCTTCAGAAGATTCTGGC
AGTGGCTGCACGGCAGTGTGTCCCCATCTTAGCTGATCACATCTATGGAG
ACATGGTGTTTTCGGATTGCAAATATGAACCACTGGCCACCCTCAGCACC
GATGTCCCCATCCTGTCCTGTGGAGGGCTGGCCAAGCGCTGGCTGGTTCC
TGGCTGGAGGTTGCCCTGGATCCTCATTCATGACCGAAGAGACATTTTTG
GCAATGAGATCCGAGATGGGCTGGTGAAGCTGAGTCAGCGCATTTTGGGA
CCCTGTACCATTGTCCAGGGAGCTCTGAAAAGCATCCTATGTCGCACCCC
CGGACAGTTTTACCACAACACTCTGAGCTTCCTCAAGTCCAATGCTGATC
TCTGTTATCCCGCGTTGGCTGCCATCCCTGGACTCCGGCCAGTCCGCCGT
TCTGCGGCTATGTACCTCATGGTTCGAATTCAGATGGAACATTTCCCACA
ATTTGAGAACGATGTGCAGTTCACCGAGCGGTTAGTTGGTCAGCAGTCTG
TCCACTGCCTCCCAGCAACGTGCTTTGAGTACCCGAATTTCATCCGAGTG
GTCATCACAGTCCCCGACGTGATGATGCTGGAGCCGTGCAGCCGGATCCA
GGAGTTCTGTGAGCAGCACTACCATTGTGCTGAAGGCAGCCAGGAGGAGT
GTGATAAATAGGCCTGCATCCATTCTCCTGAGGATGTCTCCCATCTAGGG
AAGGCTGGACTAGGCCTPGCGGCTCCTCAGGGACTCAGGTGGCCCTACTG
GGAGAGGGGCCTCAAATGCACCATGTCAAGGGTTCAAGATTGTTCCTGCT
TTTCCCCAAGTACAACCACACCCACACTCAGATCCTCCTCATTCACATCG
CAGATTACTCCCTTGCTCTCCGCTGCTAGAGTGACTCACTAATTCATTAA
TCTGCCTCCCTCTCGTAAGATTTCCTTCTTTTTTTTCTTGAAAGTACCAG
GTGAACAAAGTTTACCAGAAAGCAGTTGAGACAAGAAAATAAGAGCTCAG
GATGAGGGAAAACAAAAAGATTGACAGAATTTGTGCCCCCAACCATTTCC
TCAGACTCTAAGAAAGAACACGCTCTCTCCACGCAGGTCTCAAGCTCAAC
TCTCTTATTGCCTCACTTCAGGTATACCTCACTTTACACAATAGAATTAT
AACTGGAAAGAAGTTGGGGACACATCTATTTGGTGATTACATTTTAAACA
CATTAGGAAAAGTTGCTATTTGAACTTTTTATTGATTTTTGGGGGGAGTA
AAGAATTATTTTGGATGCAAATAAATATCCTTTAATTGATCGACTTGCCA
AATTTAGATTTGTGTGCATCAGGCTTTCTTTTTTTTCTTTTTTTAGAGAA
GTTCAATATAAGCTTTTCTTTTCTTTGTTTCTTTCTTTCTTTATTTTGAG
ATGGAGTCTTGCTCTGTCGCCCATGCTGGAGTGCAGTGGCGCGATCTCGG
CTCACTGCAACCTCCACCTCCTGGGTTCAAGCGATTCTCTTGCGTCAACG
TCCCAAGCAGTTGGGACTACAGGCGTGAGCCACCATGCCCGGCTAATRFT
TGTATTTTTAGTAGAGACAGGGTTTCACCATCTTACCCACGCTGGTCTCA
AACTCCTGACCTCAGGCAATCTGCCCGCCTGCGTCTCCTAAACTACTGGG
ATTACACCCGTCAGCCACCTCGCCCAGCGGCATCAGCCTTTCTTAAAGTG
ACAGCACCCCTGTACTACAGCAAGCAGCAATCAGAGACCTTCCAGAAATA
CTACTGTGTAAGGGCCAGAAATATCTTCACTTGTCATTGTTATATAATCA
TTATTACTTTTGCTCTAATGTTAATATTGATTTATTAATATATATTATCT
TTTCATACATTTTCTAAGAAACATTTATATTGATAAGATCTTTTATTTTG
CAAGGGCATAAATTATTGTTTTTCTTTTTTTTTTTTTTTTTTTAATAAAT
TTCACCAAGT

TABLE 5
Amino Acid sequence of Human Aminotransferase
Human Tyrosine Aminotransferase Protein Sequence:

ORF Start: 97 ORF Stop: 1459 Frame: 1
>CG135823-01-prot 454 aa
MDPYMIQMSSKGNLPSILDVHVNVGGRSSVPGKMKGRKARWSVRPSDMAK
KTFNPIRAIVDNMKVKPNPNKTMISLSIGDPTVFGNLPTDPEVTQANKDA
LDSGKYNGYAPSIGFLSSREEIASYYHCPEAPLEAKDVILTSCCSQAIDL
CLAVLANPGQNILVPRPGFSLYKTLAESMGIEVKLYNLLPEKSWEIDLKQ
LEYLIDEKTACLIVNNPSNPCGSVFSKRHLQKILAVAARQCVPILADEIY
CDMVFSDCKYEPLATLSTDVPILSCGCLAKRWLVPGWRLGWILIHDRRDI
FONEIRDGLVKLSQRILGPCTIVQGALKSILCRTPGEFYHNTLSFLKSNA
DLCYGALAAIPCLRPVRPSGANYLMVGIEMEHFPEFENDVEFIERLVAEQ
SVHCLPATCFEYPNFIRVVITVPEVNMLEACSRIQEFCEQHYHCAEGSQE
ECDK

[image]

Human Tyrosine Aminotransferase:

Locus: 16q22.1 (QTL for Intracellular Fat on 16q22)

Intracellular

Biochemistry and Cell Line Expression

Tyrosine Aminotransferase catalyses the following reaction:

L-Tyrosine+2-Oxoglutarate=4-hydroxyphenylpyruvate+L-glutamate,

using pyridoxal 5′-phosphate as a cofactor.

Tyrosine Aminotransferase activity was measured usually by fix-time assay (measurement of tyrosine absorbance by spectrophotometry). Liver extract, primary hepatocytes and different hepatocyte cell lines were reported to utilize as a source of TAT. Cell lines expressing the Tyrosine Aminotransferase can be obtained from the RTQ-PCR results shown above. These and other Tyrosine Aminotransferase expressing cell lines could be used for screening purposes.

In addition to the human version of the Tyrosine Aminotransferase identified as being differentially expressed in the experimental study, other variants have been identified by direct sequencing of cDNAs derived from many different human tissues and from sequences in public databases. No splice-form variants have been identified at CuraGen whereas several amino acid-changing cSNPs were identified in literature. Described below SNPs cause activity deficiency of TAT and were associated with disease called tyrosinemia, type II.

Natt E, Kida K, Odievre M, Di Rocco M, Scherer G.

Point mutations in the tyrosine aminotransferase gene in tyrosinemia type II.

Proc. Natl. Acad. Sci. USA Oct. 1, 1992;89(19):9297-301.

PMID: 1357662

TABLE 7
Variants of the human Tyrosine Aminotransferase obtained
from direct cloning and/or public databases.

| DNA | | | AA | | public |
|---|---|---|---|---|---|
| Position | Strand | Alleles | Position | AA Change | SNP # |
| 223 | | C:G | 74 | Ser [symbol] Stop | |
| 1086 | | G:T | 417 | Arg [symbol] Stop | |
| 1251 | | G:T | 362 | Gly [symbol] Val | |

There are several reasons to use tyrosine aminotransferase as a diagnostic and/or target for small molecule drugs and antibody therapeutics.:

1. Tyrosine Aminotransferase is a rate-limiting enzyme in phenylalanine/tyrosine catabolism, which may contribute to gluconeogenesis and lipid biosynthesis. The level of enzyme is induced by glucocorticoids, and the excess of glucocorticoids frequently results in obesity, insulin resistance and glucose intolerance.

2. Up-regulation of TAT in MB.05 study may contribute to insulin resistance in HTG rats, in MB.01—to hyperglycemia in SHR rats. Down-regulation of TAT in response to troglitazone treatment in MB.01 study suggests that TAT may be one of downstream targets for this antidiabetic drug.

3. On the other hand, down-regulation of TAT in BP24.02 study may represent the compensatory mechanism to decrease lipid biosynthesis in obese animals.

4. Taken in total, the data indicates that an inhibitor of the human Tyrosine Aminotransferase would be beneficial in the treatment of obesity.

[image]

Species #1 Rat Strains HTG, Lewis, Wistar

Species #2 Rat Strains SHR, SD

Species #3 Mouse Strains C57BL/6J

FIGS. 2A, 2B, 2C, 2D, 2E, and 2F. Differentially expressed gene fragments in rat (SPECIES #1); rat (SPECIES #2) and mouse (SPECIES #3) Tyrosine Aminotransferase. SPECIES #I. FIGS. 2A and 2B show differentially expressed gene fragments in Discovery Study MB.05 from the rat tyrosine aminotransferase (in the graphs, the abscissa is measured in lengths of nucleotides and the ordinate is measured as a signal response). A gene fragment of the rat Tyrosine Aminotransferase was initially found to be up-regulated by 1.7 fold in the muscle and liver tissues of HTG rat relative to normal control rat strain using CuraGen's GeneCalling™ method of differential gene expression. A differentially expressed rat gene fragment migrating, at approximately 145 nucleotides in length (FIG. 2A—red vertical line) was definitively identified as a component of the rat Tyrosine Aminotransferase cDNA. The method of competitive PCR was used for conformation of the gene assessment. The electropherogramatic peaks corresponding to the gene fragment of the rat Tyrosine Aminotransferase are ablated when a gene-specific primer (see below) competes with primers in the linker-adaptors during the PCR amplification. The peaks at 145 nt in length are ablated (green trace) in the sample from both the HTG and control rats.

SPECIES #2. FIGS. 2C and 2D show differentially expressed gene fragments in Discovery Study MB.01 from rat tyrosine aminotransferase (in the graphs, the abscissa is measured in lengths of nucleotides and the ordinate is measured as a signal response). The gene fragments corresponding to the rat TAT were found to be up-regulated in liver tissues of SHR rat relative to normal control rat strain, and to be down-regulated in the liver of SHR rat in response to troglitazone treatment. A differentially expressed rat gene fragment migrating, at approximately 277.4 nucleotides in length (FIG. 2C—red vertical line) was definitively identified as a component of the rat Tyrosine Aminotransferase cDNA by the method of competitive PCR. The electropherogramatic peaks corresponding to the gene fragment of the rat Tyrosine Aminotransferase are ablated when a gene-specific primer (see below) competes with primers in the linker-adaptors during the PCR amplification. The peaks at 277.4 nt in length are ablated (green trace) in the sample from both the SHR rat liver treated and untreated with troglitazone.

SPECIES #3 FIGS. 2E and 2F show differentially expressed gene fragments in Discovery Study BP24.02 from mouse tyrosine aminotransferase (in the graphs, the abscissa is measured in lengths of nucleotides and the ordinate is measured as a signal response). Additionally, gene fragments corresponding to the mouse TAT were found to be down-regulated in liver tissues of hyperglycemic fat mouse (hgsd7) relative to normal animal on low fat diet (chow) in a mouse model of dietary-induced obesity. A differentially expressed mouse gene fragment migrating, at approximately 220.3 nucleotides in length (FIG. 2A—red vertical line) was definitively identified as a component of the mouse Tyrosine Aminotransferase cDNA by the method of competitive PCR. The chromatographic peaks corresponding to the gene fragment of the mouse Tyrosine Aminotransferase are ablated when a gene-specific primer (see below) competes with primers in the linker-adaptors during the PCR amplification in the sample from both the hyperglycemic fat mouse relative and normal animals. The altered expression in of these genes in the animal model support the role of the Tyrosine Aminotransferase in the pathogenesis of obesity and/or diabetes.

[image]

[image]

[image]

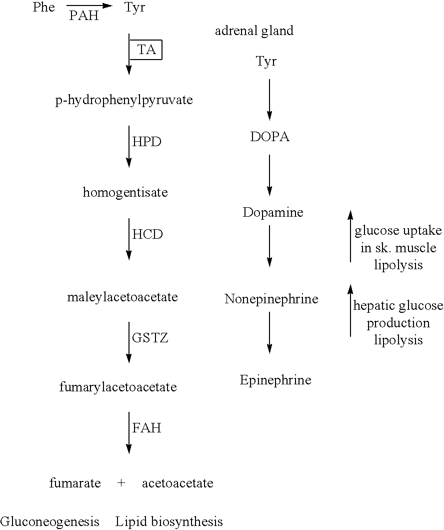

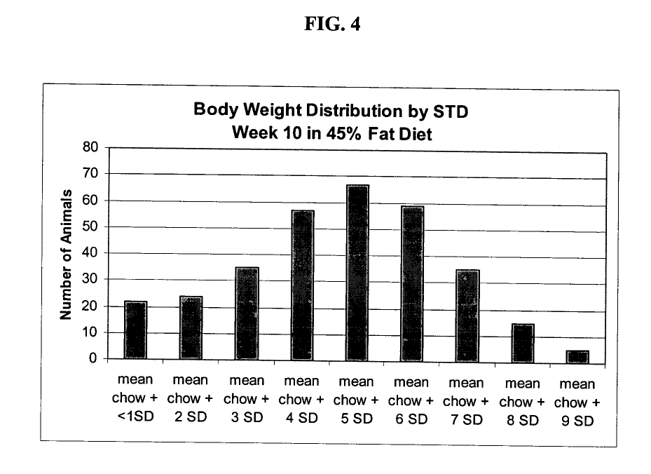

FIG. 4 shows pathways that are relevant to the etiology and pathogenesis of obesity and/or diabetes. This figure illustrates the catabolism of tyrosine and phenylalanine and suggests how alterations in expression of the human Tyrosine Aminotransferase and associated gene products function in the etiology and pathogenesis of obesity and/or diabetes. The scheme incorporates the unique findings of these discovery studies in conjunction with what has been reported in the literature. The outcome of inhibiting the action of the human Tyrosine Aminotransferase would inhibit the contribution of these catabolic pathways to gluconeogenesis and lipid biosynthesis and would be beneficial for the treatment of obesity and/or diabetes.

C. NOV13A—Human Polyamine Oxidase—CG140122-01

The present invention discloses novel associations of proteins and polypeptides and the nucleic acids that encode them with various diseases or pathologies. The proteins and related proteins that are similar to them, are encoded by a cDNA and/or by genomic DNA. The proteins, polypeptides and their cognate nucleic acids were identified by CuraGen Corporation in certain cases. The Polyamine Oxidase -encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

Discovery Process

The following sections describe the study design(s) and the techniques used to identify the Polyamine oxidase-encoded protein and any variants, thereof, as being suitable as diagnostic markers, targets for an antibody therapeutic and targets for a small molecule drugs for Obesity and Diabetes.

Studies: MB04. Mouse Obesity Model (Genetic)

Study Statements:

A large number of mouse strains have been identified that differ in body mass and composition. The AKR and NZB strains are obese, the SWR, C57L and C57BL/6 strains are of average weight whereas the SM/J and Cast/Ei strains are lean. Understanding the gene expression differences in the major metabolic tissues from these strains will elucidate the pathophysiologic basis for obesity. These specific strains of rat were chosen for differential gene expression analysis because quantitative trait loci (QTL) for body weight and related traits had been reported in published genetic studies. Tissues included whole brain, skeletal muscle, visceral adipose, and liver.

MB.08. Human Mesenchymal Stem Cell Differentiation

Bone marrow-derived human mesenchymal stem cells have the capacity to differentiate into muscle, adipose, cartilage and bone. Culture conditions have been established that permit the differentiation in vitro along the pathway to adipose, cartilage and bone. Understanding the gene expression changes that accompany these distinct differentiation processes would be of considerable biologic value. Regulation of adipocyte differentiation would have importance in the treatment of obesity, diabetes and hypertension. Human mesenchymal stem cells from 3 donors were obtained and differentiated in vitro according to published methods. RNA from samples of the undifferentiated, mid-way differentiated and fully differentiated cells was isolated for analysis of differential gene expression.

BP24.2. Diet Induced Obesity

The predominant cause for obesity in clinical populations is excess caloric intake. This so-called diet-induced obesity (DIO) is mimicked in animal models by feeding high fat diets of greater than 40% fat content. The DIO study was established to identify the gene expression changes contributing to the development and progression of diet-induced obesity. In addition, the study design seeks to identify the factors that lead to the ability of certain individuals to resist the effects of a high fat diet and thereby prevent obesity. The sample groups for the study had body weights +1 S.D., +4 S.D. and +7 S.D. of the chow-fed controls (below). In addition, the biochemical profile of the +7 S.D. mice revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues should reveal genes and pathways that can be used as therapeutic targets for obesity. The bar graph in FIG. 1 indicates results.

Polyamine Oxidase:

In multiple genecalling studies we have found the enzyme spermidine/spermine acetyl transferase to be dysregulated in various disease models (see below). This enzyme is one of the rate-limiting enzymes in the production of polyamines spermidine and spermine (see FIG. 6). FIG. 6 shows pathways where alterations in expression of the human polyamine oxidase and associated gene products function in the etiology and pathogenesis of obesity and/or diabetes. The scheme incorporates the unique findings of these discovery studies in conjunction with what has been reported in the literature. The outcome of inhibiting the action of the human polyamine oxidase would be a way to increase lypolysis by inhibiting anti-lypolytic effects of hydrogen peroxide. Previously, it was shown that oxidation of polyamines leads to generation of hydrogen peroxide, which has been shown to have antilipolytic effect of adipose and may therefore be involved in the progression of obesity. The enzyme catalyzing the reaction where hydrogen peroxide is produced, i.e. oxidation of secondary amino group of spermine, spermidine and their acetyl derivatives, is polyamine oxidase. Therefore, we nominate the enzyme polyamine oxidase as a valuable tool to inhibit the polyamine pathway and the production of hydrogen peroxide.

Rationale for Use as a Diagnostic and/or Target for Small Molecule Drugs and Antibody Therapeutics:

The following is a summary of the findings from the discovery studies, supplementary investigations and assays that also incorporates knowledge in the scientific literature. Taken in total, the data indicates that an inhibitor/antagonist of the human Polyamine oxidase would be beneficial in the treatment of obesity and/or diabetes (FIG. 5 shows biochemistry for human polyamine oxidase and assays that may be used to screen for antibody therapeutics or small molecule drugs to treat obesity and/or diabetes. Cell lines expressing the polyamine oxidase can be obtained from the RTQ-PCR results shown above. These and other polyamine oxidase-expressing cell lines could be used for screening purposes.

TABLE 1
Spermidine/spermine N-acetyltransferase Gene Sequence
identified in NZB vs SM/J mice

(Identified fragment from 206 to 616 in bold. band size: 411)

| | |
|---|---|
| 1 | GCTCCCGGGA AACGAATGAG GAACCACCTC CTCCTGCTGT TCAAGTACAG GGGCCTCGTG |
| 61 | CGCAAAGGGA AGAAAAGCAA AAGACGAAAA TGGCTAAATT TAAGATCCGT CCAGCCACTG |
| 121 | CCTCTGACTG CAGTGACATC CTGCGACTGA TCAAGGAACT GGCTAAATAT GAATACATGG |
| 181 | AAGATCAAGT CATTTTAACT GAGAAAGATC TCCAAGAGGA TGGCTTTGGA GAACACCCCT |
| 241 | TCTACCACTG CCTGGTTGCA GAAGTGCCTA AAGAGCACTG GACCCCTGAA GGACATAGCA |
| 301 | TTGTTGGGTT CGCCATGTAC TATTTTACCT ATGACCCATG GATTGGCAAG TTGCTGTATC |
| 361 | TTGAAGACTT CTTCGTGATG AGTGATTACA GAGGCTTTGG TATAGGATCA GAAATTTTGA |
| 421 | AGAATCTAAG CCAGGTTGCC ATGAAGTGTC GCTGCAGCAG TATGCACTIC TTGGTAGCAG |
| 481 | AATGGAATGA ACCATCTATC AACTTCTACA AAAGAAGAGG TGCTTCGGAT CTGTCCAGTG |
| 541 | AAGAGGGATG GAGGCTCTTC AAGATTGACA AAGAGTACTT GCTAAAAATG CCAGCAGAGG |
| 601 | AGTGAGGCGT GCCGGTGTAG ACAATGACAA CCTCCATTGT GCTTTAGAAT AATTCTCAGC |
| 661 | TTCCCTTGCT TTCTATCTTG TGTGTAGTGA AATAATAGAG CGAGCACCCA TTCCAAAGCT |
| 721 | TTATTACCAG TGACGTTGTT GCATGTTTGA AATTCGGTCT GTTTAAAGTG GCAGTCATGT |
| 781 | ATGTGGTTTG GAGGCAGAAT TCTTGAACAT CTTTTGATGA AGAACAACGT GGTATGATCT |
| 841 | TACTATATAA GAAAAACAAA ACTTCATTCT TGTGAGTCAT TTAAATGTGT ACAATCTACA |
| 901 | CACTGGTACT TAGAGTTTCT GTTTTGATTC TTTTTTTTTA AATAAACTCG CTCTTTGATT |
| 961 | T |

TABLE 2
Spermidine/spermine N-acetyltransferase Gene Sequence
identified in C57B1/6 obese euglycemic sd7 vs obese sd1

(Identified fragment from 716 to 893 in bold. band size: 178)

| | |
|---|---|
| 235 | ACCCCTTCTA CCACTGCCTG GTTGCAGAAG TGCCTAAAGA GCACTGGACC CCTCAAGCAC |
| 295 | ATAGCATTGT TGGGTTCGCC ATGTACTATT TTACCTATGA CCCATGGATT GGCAAGTTCC |
| 355 | TGTATCTTGA AGACTTCTTC GTGATGAGTG ATTACAGAGG CTTTGGTATA GGATCAGAAA |
| 415 | TTTTGAAGAA TCTAAGCCAC GTTGCCATGA AGTGTCCCTC CAGCAGTATG CACTTCTTGG |
| 475 | TAGCAGAATC CAATGAACCA TCTATCAACT TCTACAAAAC AAGAGGTGCT TCGGATCTGT |
| 535 | CCAGTGAAGA GGGATGGAGG CTCTTCAAGA TTGACAAAGA CTACTTGCTA AAAATGGCAG |
| 595 | CAGAGGAGTG AGGCQTGCCG GTGTAGACAA TGACAACCTC CATTGTGCTT TAGAATAATT |
| 655 | CTCAGCTTCC CTTCCTTTCT ATCTTGTGTG TAGTGAAATA ATAGAGCGAC CACCCATTCC |
| 715 | AAAGCTTTAT TACCAGTCAC GTTGTTGCAT GTTTGAAATT CGGTCTGTTT AAAGTGGCAG |
| 775 | TCATGTATGT GGTTTGGAGG CAGAATTCTT GAACATCTTT TGATGAAGAA CAAGGTGGTA |
| 835 | TGATCTTACT ATATAAGAAA AACAAAACTT CATTCTTGTG AGTCATTTAA ATGTGTACAA |
| 895 | TGTACACACT GGTACTTAGA GTTTCTGTTT TGATTCTTTT TTTTTAAATA AACTCCCTCT |
| 955 | TTGATTT |

TABLE 3
Spermidine/spermine N-acetyltransferase Gene Sequence
identified in human adipocyte mid-way vs undifferentiated

(Identified fragment from 162 to 355 in bold. band size: 149)

| | |
|---|---|
| 1 | CTGGTGTTTA TCCGTCACTC GCCGAGGTTC CTTGGGTCAT GCTGCCAGCC TGACTGAGAA |
| 61 | GAGGACGCTC CCGGGACACG AATGAGGAAC CACCTCCTCC TACTGTTCAA GTACAGCGGC |
| 121 | CTGGTCCGCA AAGGGAAGAA AAGCAAAAGA CGAAAATGGC TAAATTCGTG ATCCGCCCAG |
| 181 | CCACTGCCGC CGACTGCAGT GACATACTGC GGCTGATCAA GGAGCTGGCT AAATATGAAT |
| 241 | ACATGGAAGA ACAAGTAATC TTAACTGAAA AAGATCTGCT AGAAGATGGT TTTGGAGAGC |
| 301 | ACCCCTTTTA CCACTGCCTG GTTGCAGAAG TGCCGAAAGA GCACTGGACT CCGGAAGGTT |
| 361 | ACAGTCTCTA GCTTCGCCAT GTACATGGCC CTTCCGTGTA CATGGATGGG CGGGGAGGTA |
| 421 | ACTAAAAGAT CCTTTACACA ATAAAGTAGA TGATCATGAT AAATGAGGAC ACAGCATTGT |
| 481 | TGGTTTTGCC ATGTACTATT TTACCTATGA CCCGTGGATT GGCAAGTTAT TGTATCTTGA |
| 541 | GGACTTCTTC GTGATGAGTG ATTATAGAGG CTTTGGCATA GGATCAGAAA TTCTGAAGAA |
| 601 | TCTAAGCCAG GTTGCAATGA GGTGTCGCTG CAGCAGCATG CACTTCTTGG TAGCAGAATG |
| 661 | GAATGAACCA TCCATCAACT TCTATAAAAG AAGAGGTGCT TCTGATCTGT CCAGTGAAGA |
| 721 | GGGTTGGAGA CTGTTCAAGA TCGACAAGGA GTACTTGCTA AAAATGGCAA CAGAGGAGTG |
| 781 | AGGAGTGCTG CTGTAGATGA CAACCTCCAT TCTATTTTAG AATAAATTCC CAACT |

TABLE 4
Human Polyamine Oxidase (CG140122-01)
DNA and Protein Sequence

CGCCGCTCGCCGCAGACTTACTTCCCCGGCTCAGCACGGAAAGGTTCCTA
GAAGGTGAGCGCGGACGGTATGCAAAGTTGTGAATCCAGTGGTGACAGTG
CGGATGACCCTCTCAGTCGCGGCCTACGGAGAAGGGGACAGCCTCGTGTG
GTGGTGATCGGCGCCGGCTTGGCTGGCCTGGCTGCAGCCAAAGCACTTCT
TGAGCAGGGTTTCACGGATGTCACTGTGCTTGAGGCTTCCAGCCACATCG
GAGGCCGTGTGCAGAGTGTGAAACTTGGACACGCCACCTTTGAGCTGGGA
GCCACCTGGATCCATGGCTCCCATGGGAACCCTATCTATCATCTAGCAGA
AGCCAACGGCCTCCTGGAAGAGACAACCCATGGGGAACGCAGCGTGGGCC
GCATCAGCCTCTATTCCAAGAATGGCGTCGCCTGCTACCTTACCAACCAC
GGCCGCAGGATCCCCAACGACGTGGTTGAGGAATTCAGCGATTTATACAA
CCAGCTCTATAACTTGACCCAGGAGTTCTTCCGCCACGATAAACCAGTCA
ATGCTGAAAGTCAAAATAGCGTGGGGGTGTTCACCCGAGAGGAGGTGCGT
AACCGCATCAGGAATGACCCTGACGACCCAGAGGCTACCAAGCGCCTGAA
GCTCGCCATGATCCAGCAGTACCTCAACCTGGAGAGCTGTGAGAGCAGCT
CACACAGCATGGACGAGCTGTCCCTGAGCGCCTTCGGGGAGTGGACCGAG
ATCCCCGGCGCTCACCACATCATCCCCTCGGGCTTCATGCGGGTTGPGGA
GCTGCTGGCGGAGGGCATCCGTGCCCACGTCATCCAGCTAGGGAAACCTG
TCCGCTGCATTCACTGGGACCAGGCCTCAGCCCGCCCCAGAGGCCCTGAG
ATTGAGCCCCGGGGTGAGGGCGACCACAATCACGACACTGGGGAGGGTGG
CCAGGGTGGAGAGGAGCCCCGGGGGGGCAGGTGGGATGAGGATGAGCAGT
GGTCGGTGGTGGTGGAGTGCGAGGACCGTGAGCTGATCCCGGCGGACCAT
GTGATTGTGACCGTGTCGCTAGGTGTGCTAAAGAGGCAGTACACCAGTTT
CTTCCGGCCAGGCCTGCCCACAGAGAAGGTGGCTGCCATCCACCGCCTGG
CCATTGGCACCACCGACAAGATCTTTCTGGAATTCGAGGAGCCCTTCTGG
GGCCCTGAGTGCAACAGCCTACAGTTTGTGTGGCAGCACGAAGCGGAGAG
CCACACCCTCACCTACCCACCTGACCTCTGGTACCGCAAGATCTGCGGCT
TTGATGTCCTCTACCCGCCTGAGCGCTACGGCCATGTGCTGAGCGGCTGG
ATCTGCGGGGAGGAGGCCCTCGTCATGGAGAAGTGTGATGACGAGGCAGT
GGCCGAGATCTGCACGGAGATGCTGCGTCAGTTCACAGGGAACCCCAACA
TTCCAAAACCTCGGCGAATCTTGCGCTCGGCCTGGGGCAGCAACCCTTAC
TTCCGTGGCTCCTATTCATACACGCAGGTGGGCTCCAGCGGGGCGGATGT
GGAGAAGCTGGCCAAGCCCCTGCCCTACACGGAGAGCTCAAAGACAGCCC
CCATGCAGGTGCTGTTTTCCGGTCAGGCCACCCACCGCAACTACTATTCC
ACCACCCACGGTGCTCTCCTGTCCGGCCAGCGTGAGGCTGCCCGCCTCAT
TGAGATGTACCGAGACCTCTTCCACCAGGGGACCTGAGGGCTGTCCTCGC
TOCTGAGAAGAGCCACTAACTCGTGACCTCCACCCTGCCCCTTCCTCCCG
TGTGCTCCTGCCTTCCTGATCCTCTGTAGAAAGGATTTTTATCTTCTGTA
GAGCTAGCCGCCCTGACTGCCTTCAGACCTGGCCCTGTAGCTTT

TABLE 5
CG140122-01-prot 325 aa

MQSCESSGDSADDPLSRGLRRRGQPRVVVIGAGLAGLAAAKALLEQGFTD
VTVLEASSHIGGRVQSVKLGHATFELGATWIHGSHGNPIYHLAEANGLLE
ETTDGERSVGRISLYSKNGVACYLTNHGRRTPKDVVEEPSDLYNEVYNLT
QEFFRHDKPVNAESQNSVGVFTREEVRNRIRNDPDDPEATKRLKLAMIQQ
YLKVESCESSSHSMDEVSLSAFGEWTEIPGAHHIIPSGFMRVVELLAEGI
PAINTQLGKPVRCIHWDQASARPRGPEIEPRGEGDHNHDTGECGQGGEEP
RGGRWDEDEQWSVVVECEDRELIPADHVIVTVSLGVLKRQYTSFFRPGLP
TEKVAAIHRLGIGTTDKIFLEFEEPPWGPECNSLQFVWEDEAESHTLTYP
PELWYRKICGFDVLYPPERYCHVLSGWICGEEALVMEKCDDEAVAEICTE
MLRQFTGNPNIPKPRRILRSAWGSNPYFRGSYSYTQVGSSGADVEKLAKP
LPYTESSKTAPMQVLFSOEATHRKYYSTTHGALLSGQREAARLIEMYRDL
FOOGT

Table 6. Clustal W, Protein Domains, Cellular Location and Locus

The following is an alignment of the protein sequences of CG140122-01 and its alternative spliced variant CG140122-02, which are the equivalent of the public sequences AY033889 and BC000669.1, respectively. They are clustalled with the polyamine oxidase of Zea Mays, of which the structural analysis has revealed much of the domain structure of this amine oxidase. The region in bold represents the amine oxidase domain. The dotted region reprsents the signal peptide.

[image]

The variants of the human Polyamine oxidase obtained from direct cloning and/or public databases:

In addition to the human version of the Polyamine oxidase identified as being differentially expressed in the experimental study, no other variants have been identified by direct sequencing of cDNAs derived from many different human tissues and from sequences in public databases. The two alternative spliced variants (see clustalW above) are public sequences; no other splice variants have been identified at CuraGen. No SNPs have been found for polyamine oxidase. The preferred variant of all those identified, to be used for screening purposes, is CG140122-01.

[image]

Species #1 Mouse Strains NZB, SM/J, C56B1/6

Species # 2 Human

SPECIES #1 Mouse (NZB vs SM/J):

A gene fragment of the mouse spermine/spermidine N-acetyltransferase was initially found to be upregulated by 1.9 fold in the adipose of NZB mice relative to SM/J mice using CuraGen's GeneCalling™ method of differential gene expression. A differentially expressed mouse gene fragment migrating at approximately 411 nucleotides in length (FIG. 1a.—red vertical line) was definitively identified as a component of the mouse spermine/spermidine N-acetyltransferase cDNA in NZB and SM/J mouse strains. The method of competitive PCR was used for conformation of the gene assessment. The chromatographic peaks corresponding to the gene fragment of the mouse spermidine/spermine N-acetyltransferase are ablated when a gene-specific primer (see below) which competes with primers in the linker-adaptors during the PCR amplification. The peaks at 411 nt in length are ablated (green trace) in the sample from both the NZB and the SM/J mice. The altered expression in of these genes in the animal model support the role of Polyamine Oxidase in the pathogenesis of obesity and/or diabetes.

SPECIES #1 Mouse (C57B1/6 Obese Euglycemic sd7 vs Obese sd1):

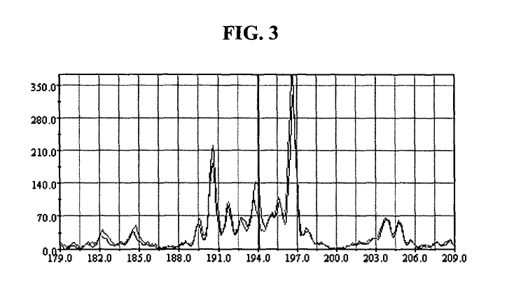

FIGS. 3A and 3B show that a differentially expressed gene fragment of the mouse spermine/spermidine N-acetyltransferase was initially found to be upregulated by 1.8 fold in the epididymal fat pad of the obese euglycemic sd7 mice relative to the obese sd1 mice using CuraGen's GeneCalling™ method of differential gene expression. A differentially expressed rat gene fragment migrating at approximately 178 nucleotides in length (FIGS. 3A and 3B—vertical line) was definitively identified as a component of the mouse spermine/spermidine N-acetyltransferase cDNA in the Troglitazone treated and the untreated SHR control rats (in the graphs, the abscissa is measured in lengths of nucleotides and the ordinate is measured as signal response). The method of competitive PCR was used for conformation of the gene assessment. The electropherogramatic peaks corresponding to the gene fragment of the mouse spermidine/spermine N-acetyltransferase are ablated when a gene-specific primer (see below) which competes with primers in the linker-adaptors during the PCR amplification. The peaks at 178 nt in length are ablated (green trace) in the sample from both the C57B1/6 obese euglycemic sd7 and obese sd1 mice. The altered expression in of these genes in the animal model support the role of Polyamine Oxidase in the pathogenesis of obesity and/or diabetes.

SPECIES #2 Human (Adipocyte Mid-Way vs Undifferentiated):

FIG. 4 shows a differentially expressed gene fragment in Discovery Study MB.08 identified in human adipocyte mid-way vs undifferentiated is from the human spermidine/spermine N-acetyltransferase A gene fragment of the human spermine/spermidine N-acetyltransferase was initially found to be upregulated by 1.6 fold in the mid-way human adipocytes relative to the undifferentiated human adipocytes using CuraGen's GeneCalling™ method of differential gene expression. A differentially expressed human gene fragment migrating at approximately 194 nucleotides in length (FIG. 3A—vertical line) was definitively identified as a component of the human spermine/spermidine N-acetyltransferase cDNA in human mid-way differentiated and undifferentiated adipocytes (in the graphs, the abscissa is measured in lengths of nucleotides and the ordinate is measured as signal response). The method of competitive PCR was used for conformation of the gene assessment. The chromatographic peaks corresponding to the gene fragment of the human spermine/spermidine N-acetyltransferase are ablated when a gene-specific primer (see below) which competes with primers in the linker-adaptors during the PCR amplification. The peaks at 194 nt in length are ablated (green trace) in the sample from both the human mid-way differentiated and undifferentiated adipocytes. The altered expression of these genes in the human cellular model support the role of Polyamine Oxidase in the pathogenesis of obesity and/or diabetes.

[image]

[image]

[image]

ODC=ornithine decarboxylase

PAO=polyamine oxidase

SSAT=spermidine/spermine N-acetyltransferase

Biochemistry of PAO:

Catalyses oxidation of secondary amino group of spermine, spermidine and their acetyl derivatives

Cofactor FAD

Monomeric

The following illustration suggests how alterations in expression of the human polyamine oxidase and associated gene products function in the etiology and pathogenesis of obesity and/or diabetes. The scheme incorporates the unique findings of these discovery studies in conjunction with what has been reported in the literature. The outcome of inhibiting the action of the human polyamine oxidase would be a way to increase lypolysis by inhibiting anti-lypolytic effects of hydrogen peroxide.

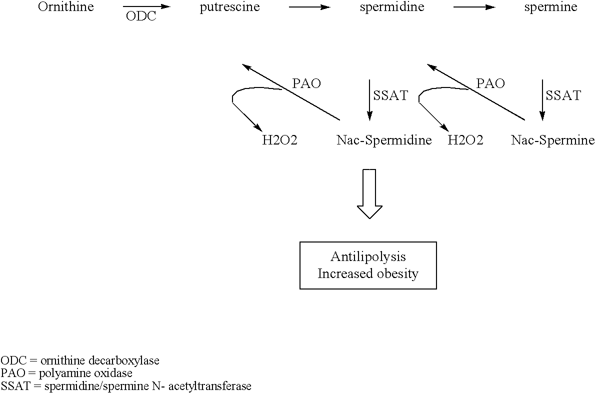

D. NOV 14a—Human Cytoplasmic Malic Enzyme—CG140316-01

The present invention discloses novel associations of proteins and polypeptides and the nucleic acids that encode them with various diseases or pathologies. The proteins and related proteins that are similar to them are encoded by a cDNA and/or by genomic DNA. The proteins, polypeptides and their cognate nucleic acids were identified by CuraGen Corporation in certain cases. The Cytoplasmic Malic Enzyme-encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

Discovery Process

The following sections describe the study design(s) and the techniques used to identify the Cytoplasmic Malic Enzyme—encoded protein and any variants, thereof, as being suitable as diagnostic markers, targets for an antibody therapeutic and targets for a small molecule drugs for Obesity and Diabetes.

Studies:

BP24.02 Dietary Induced Obesity in Mice

MB.04: Genetic Models of Obesity in Mice

Study Statements:

BP24.02: The predominant cause for obesity in clinical populations is excess caloric intake. This so-called diet-induced obesity (DIO) is mimicked in animal models by feeding high fat diets of greater than 40% fat content. The DIO study was established to identify the gene expression changes contributing to the development and progression of diet-induced obesity. In addition, the study design seeks to identify the factors that lead to the ability of certain individuals to resist the effects of a high fat diet and thereby prevent obesity. The sample groups for the study had body weights +1 S.D., +4 S.D. and +7 S.D. of the chow-fed controls (below). In addition, the biochemical profile of the +7 S.D. mice revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues should reveal genes and pathways that can be used as therapeutic targets for obesity.

MB.04: A large number of mouse strains have been identified that differ in body mass and composition. The AKR and NZB strains are obese, the SWR, C57L and C57BL/6 strains are of average weight whereas the SM/J and Cast/Ei strains are lean. Understanding the gene expression differences in the major metabolic tissues from these strains will elucidate the pathophysiological basis for obesity. These specific strains of rat were chosen for differential gene expression analysis because quantitative trait loci (QTL) for body weight and related traits had been reported in published genetic studies. Tissues included whole brain, skeletal muscle, visceral adipose, and liver.

Species #1 Mouse Strains C57BL/6

Species #2 Mouse Strains NZB, SMJ

Cytoplasmic Malic Enzyme:

This gene encodes a cytosolic, NADP-dependent enzyme that generates NADPH for fatty acid biosynthesis. The NADP-dependent malic enzyme (EC 1.1.1.40) has two forms: cytosolic and mitochondrial, that differ significantly in their activity and tissue distribution. The activity of the cytosolic enzyme, the reversible oxidative decarboxylation of malate, links the glycolytic and citric acid cycles. The reaction it catalyzes is:

Malate+NADP⁺⇄Pyruvate+CO₂+NADPH

Cytoplasmic malic enzyme is one of the anaplerotic reactions, replenishing intermediates of the citrate cycle that are utilized for biosynthesis. It also participates in the pyruvate-citrate shuttle, enabling the export of acetyl CoA from the mitochondrion to cytoplasm for fatty acid synthesis. The regulation of expression for this gene is complex. Increased expression can result from elevated levels of thyroid hormones or by higher proportions of carbohydrates in the diet.

The direct sequence of the nucleotide-long gene fragment and the gene-specific primers used for competitive PCR are indicated on the cDNA sequence of the Cytoplasmic Malic Enzyme and shown below in bold.

Competitive PCR Primer for the Mouse Cytoplasmic Malic Enzyme:

TABLE 1
Sequence Gene Sequence #1

(fragment from 1520 to 1801 in bold. band size: 282)

| | |
|---|---|
| 1039 | AAAGGACTAA TAGTTAAGGG TCGTGCATCT CTCACAGAAG AGAAAGAGGT GTTTGCCCAT |
| 1099 | GAACATGAAG AAATGAACAA TCTGGAACCC ATTGTTCAAA AGATAAAACC AACTGCCCTC |
| 1159 | ATAGGAGTTG CTGCAATTGC TGGTGCTTTC ACTCAACAAA TTCTCAAGOA TATGGCTGCC |
| 1219 | TTCAACGAGC GGCCCATCAT CTTTGCTTTC AGTAATCCGA CCAGCAAAGC GGAGTGCTCT |
| 1279 | GCAGACCACT GCTACAAGGT GACCAAGGGA CGTGCAATCT TTGCCAGCGG CAGTCCTTTT |
| 1339 | GATCCAGTCA CTCTCCCAGA TGGACGGACT CTGTTTCCTG GCCAAGGCAA CAATTCCTAC |
| 1399 | GTGTTCCCTG GAGTTGCTCT TGGGGTGGTG GCCTGCGGAC TGAGACACAT CGATGATAAG |
| 1459 | GTCTTCCTCA CCACTGCTGA GGTCATATCT CAGCAAGTGT CAGATAAACA CCTGCAAGAA |
| 1519 | GGCCGGCTCT ATCCTCCTTT GAATACCATT CGAGGCGTTT CGTTGAAAAT TGCAGTAAAG |
| 1579 | ATTGTGCAAG ATGCATACAA AGAAA&GATG GCCACTGTTT ATCCTGAACC CCAAAACAAA |
| 1639 | GAAGAATTTG TCTCCTCCCA GATGTACAGC ACTAATTATG ACCAGATCCT ACCTGATTGT |
| 1699 | TATCCGTGGC CTGCAGAAGT CCAGAAAATA CAGACCAAAG TCAACCAGTA ACGCAACAGC |
| 1759 | TAGGATTTTT AACTTTATTA GTAAAATCTT GAAGTTTTCA TGATCTTTAA CGGTCACAAT |
| 1819 | CTTTTATGAT GATTCATAGT GTGCTTACAA TAACOTCATT TTAGTTTAAT AACAAACTCA |
| 1879 | TGGGAGTCTA TTAGGATAAA TTAGGATAAA TTTCACACCA CACGGTTTTG TTTCACTTAC |
| 1939 | TGTCGATATT TATGTTTTCT CTTGTGATTA TTCTCTTTAT GAATTCTGTT TAAAAGCTAC |
| 1999 | TGTACCTCCT CCTGAGAAAG TCCTCACTGA TATGTAGGAA GCTAATGGAA GACCCACACT |
| 2059 | AGTAATAAAT TAATATAGCA TAACTTGATT ACATTTAATG CCTACAGTTC TTTCTTGACT |
| 2119 | ATTTTGCTAA AATCTCTTAA ACAGAAAAGA TAAACACAAA CTTGGGTATA GCTGAACTTT |
| 2179 | TACTAAACAG AAGCACTACT TTCTTGCCTA GAGAAAATCT TCTCAGGACT TTTATTCCAG |
| 2239 | GCCTCCGTTA GCTTTGTTCT CTTTCTACAC CTCACTCAAC ACC |

(gene length is 3105, only region from 1039 to 2281 shown)

TABLE 2
Sequence #2 Gene Sequence

(fragment from 245 to 420 in bold. band size: 176)

| | |
|---|---|
| 1 | CGCCGGGCGG CTTGGGCGGC CGCCGCCCGC CGGACTCCGC GTCCGCCCCG CCACCGGTCC |
| 61 | CAGCCATGGA GCCCCCACCC CCCCGCCGCC GACACACCCA CCAGCCCCGC TACCTCCTGA |
| 121 | CGCGGGACCC CCATCTCAAC AAGGGACTTG CTTTTAGTCT GGAAQACACA CAGCACTTGA |
| 181 | ACATTCATGG ATTGTTGCCG CCCTGCATCA TCAGCCAGGA GCTCCAGGTC CTTAGAATAA |
| 241 | TTAAGAATTT CGAACGACTG AACTCTGACT TCGACAGGTA TCTCCTGTTA ATGGACCTGC |
| 301 | AAGACAGAAA TCAGAAGCTC TTCTACAGCG TGCTCATGTC TGATGTTGAA AAGTTCATGC |
| 361 | CTATTCTTTA CACCCCCACC GTGGGCCTCG CATGCCAGCA GTACAGTTTG CCATTCCGGA |
| 421 | AGCCAAGAGG CCTCTTTATT AGTATCCATG ACAAAGGCCA CATTGCTTCA GTTCTTAATG |
| 481 | CATGGCCAGA GGATGTCGTC AACGCTATTG TGGTAACTGA TGGAGAGCGC ATCCTTGGC1 |
| 541 | TGGGAGACCT TGGCTGTAAT GGGATGGCCA TCCCTGTGCG TAAACTCGCC CTTTACACGG |
| 601 | CATGTGGAGG GGTGAACCCA CAACAGTCTC TACCCATCAC TTTGAATGTC GCAACAGAAA |
| 661 | ATGAGGAGTT ACTTAAGGAT CCACTGTACA TCGGGCTGCG GCACCGGCGA GTCAGAGGCC |
| 721 | CTGAGTATGA CGCCTTCCTG GATGAGTTCA TGGAGGCAGC GTCTTCCAAA TATGGCATGA |
| 781 | ATTGCCTTAT TCAGTTTGAA GATTTTGCCA ATCGGAATGC ATTTCGTCTC CTGAACAAGT |
| 841 | ATCGAAACAA GTATTGCACA TTTAACGATG ATATTCAAGG AACAGCGTCT GTTGCGGTTG |

(gene length is 3129, only region from 1 to 900 shown)

TABLE 3
Human Cytoplasmic Malic Enzyme Gene Sequence

>CG140316-01 2058 nt
ATGGAGCCCGAAGCCCCCCGTCGCCGCCACACCCATCAGCGCGGCTACCT
GCTGACACGGAACCCTCACCTCAACAAGCACTTGGCCTTTACCCTGGAAG
AGAGACAGCAATTGAACATTCATGGATTGTTGCCACCTTCCTTCAACAGT
CAGGAGATCCAGGTTCTTAGAGTAGTAAAAAATTTCGAGCATCTGAACTC
TGACTTTGACAGGTATCTTCTCTTAATGGATCTCCAAGATAGAAATCAAA
AACTCTTTTATAGAGTCCTGACATCTGACATTGAGAAATTCATCCCTATT
GTTTATACTCCCACTGTGGGTCTGGCTTGCCAACAATATAGTTTGGTGTT
TCCGAAGCCAAGAGGTCTCTTTATTACTATCCACCATCGAGGGCATATTG
CTTCACTTCTCAATGCATGGCCAGAAGATGTCATCAAGGCCATTGTGGTG
ACTGATGGAGAGCGTATTCTTGCCTTGGGAGACCTTGGCTGTAATGGAAT
GCCCATCCGTGTGGCTAAATTGGCTCTATATACACCTTGCGGAGGGATGA
ATCCTCAAGAATGTCTGCCTGTCATTCTGGATGTGGGAACCGAAAATCAG
GACTTACTTAAAGATCCACTCTACATTGGACTACGGCAGAGAAGAGTAAG
AGGTTCTGAATATGATGATTTTTTGGACGAATTCATGGAGGCAGTTTCTT
CCAAGTATGGCATGAATTGCCTTATTCAGTTTCAAGATTTTGCCAATGTG
AATGCATTTCGTCTCCTGAACAAGTATCGAAACCAGTATTGCACATTCAA
TGATGATATTCAAGGAACAGCATCTGTTGCACTTGCAGGTCTCCTTGCAG
CTCTTCGAATAACCAAGAACAAACTGTCTGATCAAACAATACTATTCCAA
GGAGCTGGAGAGGCTGCCCTAGGGATTGCACACCTGATTGTGATGGCCTT
GGAAAAAGAAGGTTTACCAAAAGAGAAAGCCATCAAAAAGATATGGCTGG
TTGATTCAAAAGGATTAATAGTTAAGGGACGTGCTTCCTTAACACAAGAC
AAAGACAAGTTTGCCCATGAACATGAAGAAATGAAGAACCTAGAAGCCAT
TCTTCAAGAAATAAAACCAACTCCCCTCATAGCAGTTGCTGCAATTGGTG
GTGCATTCTCAGAACAAATTCTCAAAGATATGGCTGCCTTCAATGAACCG
CCTATTATTTTTCCTTTGAGTAATCCAACTAGCAAAGCAGAATGTTCTGC
AGAGCAGTGCTACAAAATAACCAAGGGACGTGCAATTTTTGCCAGTGGCA
GTCCTTTTGATCCAGTCACTCTTCCAAATGGACAGACCCTATATCCTGGC
CAAGGCAACAATTCCTACGTGTTCCCTGGAGTTGCTCTTGGTGTTGTGGC
GTGTGGATTGAGGCAGATCACAGATAATATTTTCCTCACTACTGCTGAGG
TTATAGCTCAGCAAGTGTCAGATAAACACTTGGAAGAGGGTCGGCTTTAT
CCTCCTTTGAATACCATTAGAGATGTTTCTCTGAAAATTGCAGAAAAGAT
TGTGAAAGATGCATACCAAGAAAAGACAGCCACAGTTTATCCTGAACCGC
AAAACAAAGAAGCATTTCTCCGCTCCCAGATGTATAGTACTGATTATGAC
CAGATTCTACCTGATTGTTATTCTTGGCCTGAACAGGTGCAGAAAATACA
GACCAAAGTTGACCAGTAGGATAATAGCAAACATTTCTAACTCTATTAAT
GAGGTCTTTAAACCTTTCATAATTTTTAAAGGTTGGAATCTTTTATAATG
ATTCATAAGACACTTAGATTAAGATTTTACTTTAACAGTCTAAAAATTGA
TAGAAGAATATACGGAGAAACTCATCATTTTTATACAGGACACTAATGGG
AAGACCAAAATTACTAATAAATTTATGGTTTCTGTCTGAATTATTCTGCC
TACGTTCTCTTTAAAAGCTGTTGTACGTACTACCCAGAAACTCATCATTT
TTATACAGGACACTAATGGGAAGACCAAAATTACTAATAAATTCAAATAA
CCAACATT

TABLE 4
Amino acid sequence of Human Cytoplamic Malic
Enzyme Protein Sequence
Human Cytoplasmic Malic Enzyme Protein Sequence:

ORF Start: 1 ORF Stop: 1717 Frame: 1
>CG140316-01-prot 572 aa
MEPEAPRRRHTHQRGYLLTRNPHLNKDLAFTLEERQQLNIHGLLPPSFNS
QEIQVLRVVKNFEHLNSDFDRYLLLMDLQDRNEKLFYRVLTSDIEKFMPI
VYTPTVGLACQQYSLVFRKPRGLFITIHDRGHIASVLNAWPEDVIKAIVV
TDGERILGLGDLGCNGMGIPVGKLALYTACGGMNPQECLPVILDVGTENE
ELLKDPLYIGLRQRRVRGSEYDDFLDEFMEAVSSKYGMNCLIQFEDFANV
NAFRLLNKYRNQYCTFNDDIQGTASVAVAGLLAALRITKNKLSDQTILFQ
GAGEAALGIAHLIVMALEKEGLPKEKAIMCIWLVDSKGLIVKGRASLTQE
KEKFAHEHEEMKNLEAIVQEIKPTALIGVAAIGGAPSEQILKDMAAFNER
PIIPALSNPTSKAECSAEQCYKITRGRAIPASGSPFDPVTLPNGQTLYPG
QGNNSYVFPGVALGVVACGLRQITDNIFLTTAEVIAQQVSDKHLEEGRLY
PPLNTIRDVSLKIAEKIVKDAYQEKTATVYPEPQMKEAFVRSOMYSTDYD
QILPDCYSWPEEVQKIQTKVDQ

Table 5. Clustal W, Protein Domains, Cellular Location and Locus

The following is an alignment of the protein sequences of the human (CG140316-01), mouse (BC011081.1) and pig (X93016.1) versions of the Cytoplasmic Malic Enzyme. Also included are a variant of this enzyme cloned from liver (CG140316-02) and the mitochondrial NADP-dependent malic enzyme (X79440.1). The domain delineated by the bold line indicates the malic enzyme domain.

[image]

Human Cytoplasmic Malic Enzyme:

572aa

Locus: 6q12 (syntenic to mouse quantitative trait locus correlated with percentage of body fat. Ref: Mehrabian et al., J Clin Invest 1998; 101 (11): 2485-2496)

Intracellular

In addition to the human version of the Cytoplasmic Malic Enzyme identified as being differentially expressed in the experimental study, one other variant has been identified by direct sequencing of cDNAs derived from many different human tissues and from sequences in public databases (CG140316-02, FIG. 1C). No splice-form variants have been identified at CuraGen nor were any SNPs identified. The preferred variant of all those identified, to be used for screening purposes, is CG140316-01.

Biochemistry and Cell Line Expression:

The following illustrations summarizes the biochemistry surrounding the human Cytoplasmic Malic Enzyme and potential assays that may be used to screen for antibody therapeutics or small molecule drugs to treat obesity and/or diabetes. Generation of the reducing equivalents in form of NADPH may be coupled to enzymatic or fluorescent detection systems to provide a readout of the screening.

Malate+NADP⁺ [symbol] Pyruvate +CO₂+NADPH

Cell lines that express the Cytoplasmic Malic Enzyme include PC-3, CaCo-2 and A549, as seen in the RTQ-PCR results shown in Table 6. These and other Cytoplasmic Malic Enzyme expressing cell lines could be used for screening purposes.

Findings:

The following is a summary of the findings from the discovery studies, supplementary investigations and assays that also incorporates knowledge in the scientific literature. Taken in total, the data indicates that an inhibitor/antagonist of the human Cytoplasmic Malic Enzyme would be beneficial in the treatment of obesity and/or diabetes.

1. Cytoplasmic malic enzyme is upregulated in both liver and adipose of obese mice in different studies.

2. Upregulation of cytoplasmic malic enzyme promotes fatty acid synthesis and anaplerotic reactions replenishing TCA cycle.

3. Inhibiting cytoplasmic malic enzyme will decrease lipid synthesis and force utilization of stored fatty acids for energy generation.

4. An inhibitor of this enzyme would therefore be an effective therapeutic for obesity.

SPECIES #1 (ngsd7 vs. sd1 Liver):

FIGS. 1A and 1B show that a gene fragment of the mouse Cytoplasmic Malic Enzyme was initially found to be up-regulated by 4 fold in the liver tissues of obese mice fed a high fat diet relative to mice resistant to weight gain (on the same diet) using CuraGen's GeneCalling® method of differential gene expression. A differentially expressed mouse gene fragment migrating, at approximately 283 nucleotides in length (FIG. 1A.—vertical line) was definitively identified as a component of the mouse Cytoplasmic Malic Enzyme cDNA (in the graphs, the abscissa is measured in lengths of nucleotides and the ordinate is measured as signal response). The method of competitive PCR was used for conformation of the gene assessment. The electropherogramatic peaks corresponding to the gene fragment of the mouse Cytoplasmic Malic Enzyme are ablated when a gene-specific primer (see below) competes with primers in the linker-adaptors during the PCR amplification. The peaks at 283 nt in length are ablated (green trace) in the sample from both the obese and non-obese mice.

SPECIES #2 (NZB vs. SMJ Adipose):

FIGS. 2A and 2B show that a gene fragment of the mouse Cytoplasmic Malic Enzyme was also found to be up-regulated by 3.2 fold in the adipose of obese NZB mice relative to lean SMJ mice using CuraGen's GeneCalling® method of differential gene expression. A differentially expressed mouse gene fragment migrating, at approximately 175.9 nucleotides in length (FIG. 2A.—vertical line) was definitively identified as a component of the mouse Cytoplasmic Malic Enzyme cDNA (in the graphs, the abscissa is measured in lengths of nucleotides and the ordinate is measured as signal response). The method of competitive PCR was used for conformation of the gene assessment. The electropherogramatic peaks corresponding to the gene fragment of the mouse Cytoplasmic Malic Enzyme are ablated when a gene-specific primer (see below) competes with primers in the linker-adaptors during the PCR amplification. The peaks at 175.9 nt in length are ablated (green trace) in the sample from both the obese and non-obese mice.

[image]

[image]

E. NOV15a—Human ATP Citrate Lyase—CG142427-01, CG142427-02, CG142427-03 and CG142427-04

The present invention discloses novel associations of proteins and polypeptides and the nucleic acids that encode them with various diseases or pathologies. The proteins and related proteins that are similar to them are encoded by a cDNA and/or by genomic DNA. The proteins, polypeptides and their cognate nucleic acids were identified by CuraGen Corporation in certain cases. The ATP Citrate Lyase-encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

Discovery Process

The following sections describe the study design(s) and the techniques used to identify the ATP Citrate Lyase—encoded protein and any variants, thereof, as being suitable as diagnostic markers, targets for an antibody therapeutic and targets for a small molecule drugs for obesity and/or diabetes.

Studies:

MB.04: Lean vs. Obese Genetic Mouse Model

Study Statements:

MB.04: A large number of mouse strains have been identified that differ in body mass and composition. The AKR and NZB strains are obese, the SWR, C57L and C57BL/6 strains are of average weight whereas the SM/J and Cast/Ei strains are lean. Understanding the gene expression differences in the major metabolic tissues from these strains will elucidate the pathophysiologic basis for obesity. These specific strains of rat were chosen for differential gene expression analysis because quantitative trait loci (QTL) for body weight and related traits had been reported in published genetic studies. Tissues included whole brain, skeletal muscle, visceral adipose, and liver.

Species #1: Mouse Strains NZB vs SMJ, C57L, Cast, SWR

ATP Citrate Lyase:

ATP citrate-lyase is the primary enzyme responsible for the synthesis of cytosolic acetyl-CoA in many tissues. has a central role in de novo lipid synthesis. in nervous tissue it may be involved in the biosynthesis of acetylcholine. FIG. 1 shows a differentially expressed gene fragment from the mouse ATP Citrate Lyase.

Competitive PCR Primer for the Human ATP Citrate Lyase

Confirmatory Result—Human ATP Citrate Lyase (Discovery Study MB.04):

TABLE 1
Human ATP Citrate Lyase Gene Sequence
(Identified fragment from 1213 to 1277 in italic, band size: 65)

| | |
|---|---|
| 1 | CTGGGTTCTTTATCGATTTTACTCGATGGCCGATGCCCATGATCAGCTTCCCCTCCTTCTTCATCTTGTTGACGAACTCC |
| 81 | ATGGGAATGATGCCGCTGTCAAAGGCTTTACTGAACATCTTTGCTGCGGCATCCAAGGCACCCCCAAACCGGTCTCCAAT |
| 161 | GGTGAGCAGCCCTGAGGTGAGGCTGGAGACCACGTCCTTCCCAGCCCGAGCACAGATGATGGTATTATGGGCTCCAGAGA |
| 241 | CAGCTGGCCCGTGATCAGCTGTGACCATCAGACACATCTCAATGAACTGGCAGGAATACTTGGGCAACCTTCTCTGGAAC |
| 321 | CAGAGGAGGCCGAAGACACCACCGATCCCCATCTCCTCCTTGAAGACCTCGGTGATCGGCATCCCCGCATAAATGAGCTC |
| 401 | CTGCCCTCGCTCATCACAGATGCTGGTCATGAATGAGGCAGGTTTTCGGATCAAACCCAGCTCTCTGGCCCAAGAGTAGT |
| 481 | CCATGGGCACTGTTGGAGGTGGCACTTCCTCCCCAGGTACAATGGCTCCTTTGGCCACCAGATCTTCATACACAGACTGA |
| 561 | ATGATTTCTCCAAGCTCATCGAAGCTTCGGGGCACAAACACTCCTGCTTCCTTCAAGGCCTGGTTCTTGGCTACTGCAGT |
| 641 | TTCAGAAGTCTGGTTGGCACAAGCTCCAGCATGGCCAAACTCGACCTCGGAGCAGAACATGGTGGCACAGGTCCCGATAC |
| 721 | ACCAGCAGACCACTGGCTTGGTGAGGCGGCCCTCCTTGATGCCCCGGCAGATCTTATATTCCTCTCTGCCCCCTATCTCC |
| 801 | CCAAGAACTACGATCATCTTGACTCCTGGAGTGTCCTGGTAGCGCAGCACGTGATCCATGAATGTGGACCCAGGGTACCT |
| 881 | GTCCCCGCCGATGGCCACGCCCTCATAGACACCATCTGTGGTCCGGGAGATGATGTTATTGAGTTCATTAGACATGCCTC |
| 961 | CTGAACGTGAGACGTAGGCCACGCTGCCTGGGCGCTACAGTTTGGAGGCCAGGATGTTGTCCAGCA |

TABLE 2
Nucleotide and pTotein sequence of Human ATP CitTate Lyase
CG142427-01

GGCACGAGGCCCGCACAAAAGCCGGATCCCGGGAAGCTACCGGCTGCTGGGGTGCTCCGGATTTTGCGGG
GTTCGTCGGGCCTGTGGAAGAAGCGCCGCGCACCGACTTCGGCAGAGGTAGAGCAGGTCTCTCTCCAGCC
ATGTCGGCCAAGGCAATTTCAGAGCAGACGGGCAAAGAACTCCTTTACAAGTTCATCTGTACCACCTCAG
CCATCCAGAATCGCTTCAACTATGCTCGGGTCACTCCTGACACACACTCGGCCCGCTTGCTCCAGGACCA
CCCCTGGCTGCTCAGCCAGAACTTGGTAGTCAAGCCAGACCAGCTGATCAAACCTCGTCGAAAAGTTGGT
CTCGTTGGGGTCAACCTCACTCTGGATGGGGTCAAGTCCTGGCTGAAGCCACGGCTGGGACAGGAAGCCA
CAGTTCGCAACGCCACAGCCTTCCTCAAGAACTTTCTGATCGAGCCCTTCGTCCCCCACAGTCAGGCTGA
GGAGTTCTATGTCTGCATCTATGCCACCCGAGAAGGGGACTACGTCCTGTTCCACCACGAGGGGGGTGTG
GACGTGGGTGATGTGGACGCCAAGGCCCAGAAGCTGCTTGTTGGCGTGGATGAGAAACTGAATCCTGAGG
ACATCAAAAAACACCTGTTGGTCCACGCCCCTGAAGACAAGAAAGAAATTCTGGCCAGTTTTATCTCCGG
CCTCTTCAATTTCTACGAGGACTTGTACTTCACCTACCTCGAGATCAATCCCCTTGTAGTGACCAAAGAT
GGAGTCTATGTCCTTGACTTGGCGGCCAAGGTGGACGCCACTGCCGACTACATCTGCAAAGTGAAGTGGG
GTGACATCCAGTTCCCTCCCCCCTTCGGGCGGGAGGCATATCCAGAGGAAGCCTACATTGCAGACCTCGA
TGCCAAAAGTGGGGCAAGCCTGAAGCTGACCTTGCTGAACCCCAAAGGGAGGATCTGGACCATGCTGCCC
GGGGGTGGCGCCTCTGTCGTGTACAGCGATACCATCTGTGATCTAGGGGGTGTCAACGACCTGGCAAACT
ATGGGGAGTACTCAGGCGCCCCCAGCGAGCAGCAGACCTATGACTATGCCAAGACTATCCTCTCCCTCAT
GACCCGAGAGAAGCACCCAGATGGCAAGATCCTCATCATTGCACGCAGCATCGCAAACTTCACCAACGTG
GCTGCCACGTTCAAGGGCATCGTGAGAGCAATTCCAGATTACCAGGGCCCCCTGAAGGAGCACGAAGTCA
CAATCTTTGTCCGAAGAGGTGGCCCCAACTATCAGGAGGGCTTACGGGTGATGGGAGAAGTCGGGAAGAC
CACTGGGATCCCCATCCATGTCTTTCGCACAGACACTCACATGACGGCCATTCTGGGCATGGCCCTGGGC
CACCCGCCCATCCCCAACCAGCCACCCACAGCGCCCCACACTGCAAACTTCCTCCTCAACGCCAGCGGGA
GCACATCGACGCCAGCCCCCACCAGGACACCATCTTTTTCTGAGTCCAGGGCCGATGAGGTGGCGCCTGC
AAAGAAGGCCAAGCCTGCCATGCCACAAGATTCAGTCCCAAGTCCAAGATCCCTGCAAGGAAAGAGCACC
ACCCTCTTCAGCCGCCACACCAAGGCCATTGTGTGGGGCATGCAGACCCGGGCCGTGCAAGGCATGCTGG
ACTTTGACTATGTCTGCTCCCGAGACGAGCCCTCAGTGGCTCCCATCGTCTACCCTTTCACTGGGGACCA
CAAGCAGAAGTTTTACTGGGGGCACAAAGAGATCCTGATCCCTGTCTTCAAGAACATGGCTGATGCCATG
AGGAAGCATCCCGAGGTAGATGTGCTCATCAACTTTGCCTCTCTCCGCTGTGCCTATGACAGCACCATGG
AGACCATGAACTATGCCCAGATCCGGACCATCGCCATCATAGCTGAAGGCATCCCTGAGGCCCTCACGAG
AAAGCTGATCAAGAAGGCGGACCAGAACGGAGTGACCATCATCGGACCTGCCACTGTTGGAGGCATCAAG
CCTGCGTGCTTTAAGATTCGCAACACAGGTGGGATCCTGCACAACATCCTGGCCTCCAAACTGTACCGCC
CAGGCAGCGTGGCCTATGTCTCACGTTCCGGAGGCATGTCCAACGAGCTCAACAATATCATCTCTCGGAC
CACGGATGGCGTCTATGAGGGCGTGGCCATTGGTGGGGACAGGTACCCGGGCTCCACATTCATGGATCAT
GTGTTACGCTATCAGGACACTCCACCAGTCAAAATGATTGTGGTTCTTGGAGAGATTGGGGGCACTGAGG
AATATAACATTTGCCGGACCATCAAGGAGGGCCGCCTCACTAAGCCCATCGTCTGCTGGTGCATCGGGAC
GTGTGCCACCATGTTCTCCTCTGAGGTCCAGTTTGGCCATGCTGGAGCTTGTGCCAACCAGGCTTCTGAA
ACTGCAGTAGCCAAGAACCAGGCTTTGAAGGAAGCAGGAGTGTTTGTGCCCCCGAGCTTTGATCACCTTG
CAGAGATCATCCACTCTGTATACGAAGATCTCGTGGCCAATGGAGTCATTGTACCTGCCCAGGACGTGCC
GCCCCCAACCCTGCCCATGGACTACTCCTGGGCCACGGAGCTTCGTTTGATCCGCAAACCTCCCTCGTTC
ATGACCAGCATCTGCGATGAGCGAGGACAGGAGCTCATCTACGCGGGCATGCCCATCACTGAGGTCTTCA
AGGAAGAGATGGGCATTGGCGGGGTCCTCGGCCTCCTCTCGTTCCAGAAAAGGTTGCCTAAGTACTCTTG
CCAGTTCATTGAGATGTGTCTGATGGTGACAGCTGATCACCGCCCAGCCCTCTCTGGAGCCCACAACACC
ATCATTTGTGCGCGAGCTGGGAAAGACCTGGTCTCCAGCCTCACCTCGGGGCTGCTCACCATCGGGGATC
GGTTTGGGGGTGCCTTGGATGCAGCACCCAAGATGTTCAGTAAAGCGTTTGACAGTGGCATTATCCCCAT
GGAGTTTGTGAACAACATCAAGAAGGAAGGGAAGCTGATCATGGGCATTGGTCACCGAGTGAAGTCCATA
AACAACCCAGACATCCCAGTGCAGATCCTCAAAGATTACGTCAGGCAGCACTTCCCTGCCACTCCTCTGC
TCGATTATGCACTGGAAGTAGAGAAGATTACCACCTCGAAGAAGCCAAATCTTATCCTGAATGTAGATGG
TCTCATCGGAGTCGCATTTGTAGACATGCTTAGAAACTGTGGGTCCTTTACTCGCGAGGAAGCTGATGAA
TATATTGACATTGGAGCCCTCAATGGCATCTTTGTGCTGCGAAGGAGTATGGGGTTCATTGGACACTATC
TTGATCAGAAGAGGCTGAAGCAGGGGCTCTATCGTCATCCGTGGGATGATATTTCATATGTTCTTCCGGA
ACACATGAGCATGTAACAGAGCCAGGAACCCTACTGCACTAAACTGAAGACAAGATCTCTTCCCCCAAGA
AAAAGTGTACAGACAGCTGGCAGTGGAGCCTGCTTTATTTAGCAGGGGCCTGGAATGTAAACAGCCACTG
GGGTACACGCACCGAACACCAACATCCACAGGCTAACACCCCTTCAGTCCACACAAAGAAGCTTCATATT
TTTTTTATAAGCATAGAAATAAAAACCAAGCCAATATTTGTGACTTTGCTCTGCTACCTGCTGTATTTAT
TATATCGAAGCATCTAAGTACTGTCAGGATGGGGTCTTCCTCATTGTAGGGCCTTAGGATGTTGCTTTCT
TTTTCCATTAGTTAAACATTTTTTTCTCCTTTGGAGGAAGGGAATGAAACATTTATGGCCTCAAGATACT
ATACATTTAAAGCACCCCAATGTCTCTCTTTTTTTTTTTTTACTTCCCTTTCTTCTTCCTTATATAACAT
GAAGAACATTGTATTAATCTGATTTTTAAAGATCTTTTTGTATGTTACGTGTTAACGGCTTGTTTGCTAT
CCCACTGAAATGTTCTGTGTTGCAGACCAGAGTCTGTTTATGTCAGGGGGATGGGGCCATTGCATCCTTA
GCCATTGTCACAAAATATGTGGAGTAGTAACTTAATATGTAAAGTTGTAACATACATACATTTAAAATGG
AAATGCAGAAAGCTGTGAAATGTCTTGTGTCTTATGTTCTCTGTATTTATGCAGCTGATTTGTCTGTCTG
TAACTGAAGTGTGGGTCCAAGGACTCCTAACTACTTTGCATCTGTAATCCACAAAGATTCTGGGCAGCTG
CCACCTCAGTCTCTTCTCTGTATTATCATAGTCTGGTTTAAATAAACTATATAGTAACAAAAAAAAAAAA
AAAAAAAAAAAAAAAAAAAAAAAAAAAAAAAAAAAAAAAAAAAAAAAAAAAAAAAAAAAAAAAAAAAAAA
AAAAAAAAAAAAAAAAA

TABLE 3
Amino acid sequence of Human ATP Citrate Lyase
ORF StaTt: 141 GRF Stop: 3444 Frame: 3
Human ATP Citrate Lyase Protein Sequence:
CG142427-01-prot 1101 aa

MSAKAISEQTGKELLYKFICTTSAIQNRFKYARVTPDTDWARLLQDHPWLLSQNLVVKPD
QLIKRRGKLGLVGVNLTLDGVKSWLKPRLGQEATVGKATGFLKNFLIEPFVPHSQAEEFYV
CIYATREGDYVLFHHEGGVDVGDVDAKAQKLLVGVDEKLNPEDIKKHLLVHAPEDKKEIL
ASFISGLFNFYEDLYFTYLEINPLVVTKDGVYVLDLAAKVDATADYICKVKWGDIEFPPPFG
REAYPEEAYIADLDAKSGASLKLTLLNPKGRIWTMVAGGGASVVYSDTICDLGGVNELAN
YGEYSGAPSEQQTYDYAKTILSLMTREKHPDGKILIIGGSTANFTNVAATFKGIVRAIRDYQ
GPLKEHEVTIFVRRGGPNYQEGLRVMGEVGKTTGIPIHVFGTETHMTAIVGMALGHRPIPN
QPPTAAHTANFLLNASGSTSTPAPSRTASFSESRADEVAPAKKAKPAMPQDSVPSPRSLQG
KSTTLFSRHTKAIVWGMQTRAVQGMLDFDYVCSRDEPSVAAMVYPFTGDHKQKFYWGH
KEILIPVFKNMADAMRKHPEVDVLINFASLRSAYDSTMETMNYAQIRTIAIIAEGIPEALTRK
LIKKADQKGVTIIGPATVGGIKIPGCFKIGNTGGMLDNILASKLYRPGSVAYVSRSGGMSNEL
NNHSRTTDGVYEGVATGGDRYPGSTFMDHVLRYQDTPGVKMIVVLGEIGGTEEYKICRGIK
EGRLTKPIVCWCIGTCATMFSSEVQFGHAGACANQASETAVAKNQALKEAGVFVPRSFDE
LGEIIQSVYEDLVANGVIVPAQEVPPPTVPMDYSWARELGLIRKLPASFMTSICDERGQELIY
AGMPITEVFKEEMGIGGVLGLLWFQKRLPKYSCQFIEMCLMVTADHGPAVSGAHNTIICAR
AGKDLVSSLTSGLLTIGDRFGGALDAAAKMFSKAFDSGIIPMEFVNKMKKEGKLIMGIGHR
VKSINNPDMRVQILKDYVRQHFPATPLLDYALEVEKITTSKKPNLILNVDGLIGVAFVDML
RNCGSFTREEADEYIDLGALNGIFVLGRSMGFIGHYLDQKRLKQGLYRHPWDDISYVLPEH
MSM

[image]

Human ATP Citrate Lyase

1105 amino acids; 121 kd

Locus: 17q12-q21

Intracellular (Cytoplasmic)

In addition to the human version of the ATP Citrate Lyase identified as being differentially expressed in the experimental study, other variants have been identified by direct sequencing of cDNAs derived from many different human tissues and from sequences in public databases. No splice-form variants have been identified at CuraGen whereas several amino acid-changing cSNPs were identified. These are found below. The preferred variant of all those identified, to be used for screening purposes, is CG 142427-01.

TABLE 5
The variants of the human ATP Citrate Lyase obtained
from direct cloning and/or public databases

| DNA | | | AA | AA | public |
|---|---|---|---|---|---|
| Position | Strand | Alleles | Position | Change | SNP# |
| 363 | Plus | A:G | 75 | Asn=>Asp | rs1058875 |
| 665 | Plus | A:C | 175 | Glu=>Asp | rs2304497 |
| 2318 | Plus | G:A | 726 | Lys=>Lys | rs1802731 |
| 2377 | Plus | G:A | 746 | Gly=>Glu | rs1802730 |
| 2756 | Plus | C:T | 873 | Leu=>Leu | rs2277697 |
| 3308 | Plus | C:G | 1056 | Ala=>Ala | Rs1802732 |

Biochemistry and Cell Line Expression

The following summarizes the biochemistry surrounding the human ATP Citrate Lyase enzyme: ATP Citrate Lyase catalyzes the conversion of Citrate plus CoA in the presence of ATP into orthophosphate+Acetyl CoA+Oxaloacetate with a release of ADP. Acetyl CoA can then be used as a substrate for Fatty Acid synthesis.

Cell lines expressing the ATP Citrate Lyase enzyme can be obtained from the RTQ-PCR results shown above. These and other ATP Citrate Lyase enzyme expressing cell lines could be used for screening purposes.

Findings:

An inhibitor to ATP Citrate Lyase will force Acetyl CoA to be produced by alternative pathways, thus decreasing the available pool for fatty acid and triglyceride synthesis. The decreased pool of Acetyl CoA will cause a down-regulation of the Cholesterol biosynthetic pathway preventing excess production of LXRa ligands

Taken in total, the data indicates that an inhibitor of the human ATP Citrate Lyase enzyme would be beneficial in the treatment of obesity and/or diabetes.

Sequences: The sequence of Acc. No. CG142427-01 is an In silico prediction based on sequences available in CuraGen's proprietary sequence databases or in the public human sequence databases, and provided either the full length DNA sequence, or some portion thereof.

SPECIES #1 A gene fragment of the mouse ATP Citrate Lyase was initially found to be up-regulated by 2 fold in the adipose tissues of the NZB mouse relative to the SMJ mouse strain using CuraGen's GeneCalling™ method of differential gene expression. Similar results were found in adipose in NZB vs C57L, Cast and SWR mouse strains (All were up-regulated; 2.7×, 5×, and 2.4× respectively). A differentially expressed mouse gene fragment migrating, at approximately 161.7 nucleotides in length (FIG. 1A and 1B.—vertical line) was definitively identified as a component of the mouse ATP Citrate Lyase cDNA (in the graphs, the abscissa is measured in lengths of nucleotides and the ordinate is measured as signal response). The method of competitive PCR was used for conformation of the gene assessment. The chromatographic peaks corresponding to the gene fragment of the rat ATP Citrate Lyase are ablated when a gene-specific primer (see below) competes with primers in the linker-adaptors during the PCR amplification. The peaks at 161.7 nt in length are ablated in the sample from both the NZB and SMJ mice.

The direct sequence of the 65 nucleotide-long gene fragment and the gene-specific primers used for competitive PC are indicated on the complete cDNA sequence of the ATP Citrate Lyase and shown below in bold. The gene-specific primers at the 5′ and 3′ ends of the fragment are in bold.

[image]

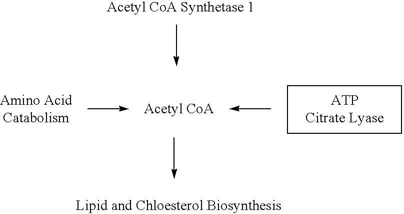

F. NOV16a—Human Serine Dehydratase—CG142631-01

Discovery Process

The following sections describe the study design(s) and the techniques used to identify the Serine Dehydratase—encoded protein and any variants, thereof, as being suitable as diagnostic markers, targets for an antibody therapeutic and targets for a small molecule drugs for obesity and/or diabetes.

Studies:

MB.01: Insulin Resistance in Rat

Study Statements:

MB.01: The spontaneously hypertensive rat (SHR) is a strain exhibiting features of the human Metabolic Syndrome X. The phenotypic features include obesity, hyperglycemia, hypertension, dyslipidemia and dysfibrinolysis. Tissues were removed from adult male rats and a control strain (Wistar-Kyoto) to identify the gene expression differences that underlie the pathologic state in the SHR and in animals treated with various anti-hyperglycemic agents such as troglitizone. Tissues included sub-cutaneous adipose, visceral adipose and liver.

Species #1 Rat Strains SHR

Serine Dehydratase:

Serine dehydratase catalyzes the PLP-dependent alpha, beta-elimination of L-serine to pyruvate and ammonia. It is one of three enzymes that are regarded as metabolic exits of the serine-glycine pool. Serine dehydratase is found predominantly in the liver.

TABLE 1
Competitive PCR Primer for the Human Serine Dehydratase
Confirmatory Result-Human Serine Dehydratase (Discovery Study MB.01):
(Identified fragment from 221 to 545 in italic, band size: 325)

| | |
|---|---|
| 1 | GCTTTATAAACATATATATATTAATTTTTATTTACAATGAAAAAGTGCATATTATAAACATGGATAAAGGAGGGTGGGCC |
| 81 | ACTGTCAGGGJGACGCCCACCCAGCCTACTCAGGGGTGCTGGTGACCCCTCAGGGTGGCCAGGGCAGCAGCAGATATCAC |
| 161 | TTGAGTAGCTCATTCAGGCCCAGCTGTCCCTTGAGTGCCTGCAGCTGTGCCAGGCTGATGTTGCTGCCACCACACACAAT |
| 241 | GACAACCAGCGAGGCCAGTGGGGTTTGCAGTCGGGCCTCAGCCTGCACCCTCCACACCACACCGCTGTACACTGCAGCCA |
| 321 | GGGCAGCGCCACACGCGGGCTCCACCACGATCTTCTCATCGTCTACGAACTTCTCGATACCAGTCACAGCCTCCTGGTCT |
| 401 | GAGATGACCTCAGAGAAAATGGGGTGTTCGTAAAACAGCTTCAGGGTCTGTGCCCCCACAGTGTTCACACCCAAGGCCTT |
| 481 | GGCAACACTGGTGATCTTGGCCAGGGTGACCAGCTTTCCTTCCTTGACGGCAGCGTGGAAGCTGTGGGCGCCGAAGGTCT |
| 561 | CCATGGCGATGATGCGCACATCCTCCCAGCCCACCTCCCCCAGCCCCTGGACCACTCCGCACAGCAGGCCTCCACCGCCC |
| 641 | ACAGACAGCACAATGGCCCCGGGCTTGGCGCTCAGTGTCTCCTTCAGCTCCTTCACAAGGGAAGTGTGGCCTTCCCAGAT |
| 721 | GAGAGGGTCATCGAAGGGGGAGATGTACACCCAACCTGGGTTGTTCTTTTCCAGAGCCTTCGCCAGTTGCATGGCCTCAT |
| 801 | CCAGCATCTCTCCCACCACTTCAACTGTGGCCCCTTCGTTCTTCAGCCGCTCAATGGTGAGGGCAGGTGTGGTGCTTGGC |
| 881 | ACAACAATAGTGGCTCGGAGGCCCAGCCTCCTGGCAGCATAGGCAGTCGCCATGCCCGCGGTGCCCGCTGAAGAGCACAC |
| 961 | GAAATGTTTACAGCCTTGTTTTGCCTTCATCTTGCAGAGATGCCCAATGCCTCGCATCTTGAAGGAGCCAGAGGGCTGAG |
| 1041 | AGCTGTCCATCTTAAGGAACAGACTAGTGCCGGCCACTTTGGACAATGCCATGGTGTCACGTAGTCGCGTCTTCACGTGC |
| 1121 | AGGGACTCCTGGGCAGCCATGGCATGTAGCTTTGAAGGTTGGATCCTCCTGTCTCAGTCTCCCAATTGCTGGGATCACAG |
| 1201 | GTATGCCCCGCCGCACCCGGCACAGGAGGAGCTGGACACAGCGAGCGAGAAGGGTAGATTTTGTCTGTGTCCTGGGAGAG |
| 1281 | TGGAAAGT |

TABLE 2
Nucleotide and protein sequence of Human Serine Dehydratase, CG
142631-01

CCTTCTCTTCGTGGGCTATCTACTCAGTTGATCCCTCCCTCGCTGGCTTGGCTCTGACTCCTG
CTCAGACCCATCACCTTTGCCGGGGAATGATGTCTGGAGAACCCCTGCACGTGAAGACCCCC
ATCCGTGACAGCATGGCCCTGTCCAAAATGGCCGGCACCAGCGTCTACCTCAAGATGGACAG
TGCCCAGCCCTCCGGCTCCTTCAAGATCCGGGGCATTGGGCACTTCTGCAAGAGGTGGGCCA
AGCAAGGCTGTGCACATTTTGTCTGCTCCTCGGCGGGCAACGCAGGCATGGCGGCTGCATAT
GCGGCCAGGCAACTCGGCGTCCCCGCCACCATCGTAGTGCCCGGCACCACACCTGCTCTCA
CCATTGAGCGCCTCAAGAATGAAGGTGCCACATGCAAGGTGGTGGGTGAGTTATTGGATGAA
GCCTTCGAGCTGGCCAAGGCCCTAGCGAAGAACAACCCGGGTTGGGTCTACATTCCCCCCTT
TGATGACCCCCTCATCTGGGAAGGCCACGCTTCCATCGTGAAAGAGCTGAAGGAGACACTGT
GGGAAAAGCCGGGGGCCATCGCGCTGTCAGTGGGCGGCGGGGGCCTGCTGTGTGGAGTGG
TCCAGGGGCTGCAGGAGTGTGGCTGGGGGGACGTGCCTGTCATCGCCATGGAGACTTTTGGT
GCCCACAGCTTCCACGCTGCCACCACCGCAGGCAAACTTGTCTCCCTGCCCAAGATCACCAG
TGTTGCCAAGGCCCTGGGCGTGAAGACTGTGGGGTCTCAGGCCCTGAAGCTGTTTCAGGAAC
ACCCCATTTTCTCTGAAGTTATCTCGGACCAGGAGGCTGTGGCCGCCATTGAGAAGTTCGTGG
ATGATGAGAAGATCCTGGTGGAGCCCGCCTGGGGCGCAGCCCTGGCCGCTGTCTATAGCCAC
GTGATCCAGAAGCTCCAACTGGAGGGGAATCTCCGAACCCCGCTGCCATCCCTCGTGGTCAT
CGTCTGCGGGGGCAGCAACATCAGCCTGGCCCAGCTGCGGGCGCTCAAGGAACAGCTGGGC
ATGACAAATAGGTTGCCCAAGTGAGGACGGACCCCTTACCGATCTGTGCTCTCCTAGCCCAAG
AGACCCCTGGAGGGGCTGGAGTTTATCCAGCGCCTCGTCGTATGTTTGGCTGAGCACCTGTG
GCCCTGGGTGCAGGTTAACTTCTTGTTATCAGGAGCCCACTATGCAGAGGCCAAAGGTCGGC
AGCCAGCGAGGCTATGAATTGGACCTTTTTGGTATCTGTGTGACTGCTCTGTGCCCATCCTTA
GCCAACTTGCTGGCGTGACAAGTGCCCACAAGTAACACACCAGGTACCCAGAGCAGGGTGGA
CAGGAGAGACCTGAATCACAGCAGTGAGG

TABLE 3
ORF Start: 90 ORF Stop: 1074 Frame: 3
Human Serine Dehydratase Protein Sequence:
CG142631-01-prot 328 aa

MMSGEPLHVKTPIRDSMALSKMAGTSVYLKMDSAQPSGSFKIRGIGHFCK
RWAKQGCAHFVCSSAGNAGMAAAYAARQLGVPATIWPGTTPALTIERLKN
EGATCKWGELLDEAFELAKALAKNNPGWVYIPPFDDPLIWEGHASIVKEL
KETLWEKPGAIALSVGGGGLLCGWQGLQECGWGDVPVIAMETFGAHSFHA
ATTAGKLVSLPKITSVAKALGVKTVGSQALKLFQEHPIFSEVISDQEAVA
AIEKFVDDEKILVEPAWGMLAAVYSHVIQKLQLEGNLRTPLPSLVVIVCG
GSNISLAQLRALKEQLGMTNRLPK

[image]

Human Serine Dehydratase

328 amino acids; 34 kd

Locus: 12

Intracellular

In addition to the human version of the Serine Dehydratase identified as being differentially expressed in the experimental study, other variants have been identified by direct sequencing of cDNAs derived from many different human tissues and from sequences in public databases. No splice-form variants have been identified at CuraGen whereas several amino acid-changing cSNPs were identified. These are found below. The preferred variant of all those identified, to be used for screening purposes, is CG142631-01.

TABLE 5
The variants of the human Serine Dehydratase obtained
from direct cloning and/or public databases

| DNA | | | AA | AA | public |
|---|---|---|---|---|---|
| Position | Strand | Alleles | Position | Change | SNP # |
| 777 | Plus | G:T | 230 | Ala => Ser | rs1050062 |

Biochemistry:

The following illustrations summarizes the biochemistry surrounding the human Serine Dehydratase enzyme. L-Serine is converted to Pyruvate by pyridoxal phosphate requiring Serine Dehydratase with the release of ammonia as a by product. Pyruvate is a primary substrate in the process of gluconeogenesis. Cell lines expressing the Serine Dehydratase enzyme can be obtained from the RTQ-PCR results shown above. These and other Serine Dehydratase enzyme expressing cell lines could be used for screening purposes.

Findings:

Serine Dehydratase (SDH) is critical for gluconeogenesis. In models of Diabetes SDH is up-regulated and in studies utilizing TZDs expression of SDH is down-regulated. An inhibitor of this enzyme would decrease glucose production. By improving daily blood glucose levels and maintaining HbA1c at or below 7.5 may prevent many diabetic complications.

Taken in total, the data indicates that an inhibitor of the human Serine Dehydratase enzyme would be beneficial in the treatment of obesity and/or diabetes.

Sequences

The sequence of Acc. No. CG142631-01 is an In silico prediction based on sequences available in CuraGen's proprietary sequence databases or in the public human sequence databases, and provided either the full length DNA sequence, or some portion thereof.

[image]

G. NOV53a—Human Plasma Kallikrein—CG56155-01

Discovery Process

The following sections describe the study design(s) and the techniques used to identify the Plasma Kallikrein—encoded protein and any variants, thereof, as being suitable as diagnostic markers, targets for an antibody therapeutic and targets for a small molecule drugs for Obesity and Diabetes.

MB.01: Metabolic Syndrome X in Rat

MB.04: Mouse Obesity

Study Statements:

MB.01 The spontaneously hypertensive rat (SHR) is a strain exhibiting features of the human Metabolic Syndrome X. The phenotypic features include obesity, hyperglycemia, hypertension, dyslipidemia and dysfibrinolysis. Tissues were removed from adult male rats and a control strain (Wistar-Kyoto) to identify the gene expression differences that underlie the pathologic state in the SHR and in animals treated with various anti-hyperglycemic agents such as troglitizone. Tissues included sub-cutaneous adipose, visceral adipose and liver.

MB.04 A large number of mouse strains have been identified that differ in body mass and composition. The AKR and NZB strains are obese, the SWR, C57L and C57BL/6 strains are of average weight whereas the SM/J and Cast/Ei strains are lean. Understanding the gene expression differences in the major metabolic tissues from these seatrains will elucidate the pathophysiologic basis for obesity. These specific strains of rat were chosen for differential gene expression analysis because quantitative trait loci (QTL) for body weight and related traits had been reported in published genetic studies. Tissues included whole brain, skeletal muscle, visceral adipose, and liver.

Species #1 Rat Strains SHR, WKY

Species #2 Mouse Strains C57BL, Cast/Ei

Plasma Kallikrein:

Plasma Kallikrein (PK) has been shown to activate specifically plasminogen during adipose differentiation. Plasminogen activation, followed by fibrinolysis, has been implicated in adipose differentiation by remodeling of the fibronectin-rich extracellular matrix of preadipocytes.

TABLE 1
SPECIES #1 Rat Plasma Kallikrein Gene Fragment used for competitive
PCR

| | |
|---|---|
| 1035 | TCCCCAAGAC TGCAAGCCAC AGGGCTCTAA ATGTTCCTTA AGGTTATCCA CGGATGGCTC |
| 1095 | TCCAACTAGG ATCACCTATC AGCCACACGG GAGCTCTGGT TATTCTCTGA GACTGTGTAA |
| 1155 | AGTTGTGGAG AGCTCTCACT CTACGACAAA AATAAATGCA CGTATTGTGG GAGGAACAAA |
| 1215 | CTCTTCTTTA GGAGAGTGGC CATGGCAGGT CAGCCTGCAA GTGAAGTTGG TTTCTCAGAA |
| 1275 | CCATATGTGT GGAGGGTCCA TCATTGGACG CCAATCCATA CTGACGGCTG CCCATTGCTT |
| 1335 | TGATGGGATT CCCTATCCAG ACGTGTGGCG TATATATGGC GGGATTCTTA ATCTGTCAGA |
| 1395 | GATTACAAAC AAAACGCCTT TCTCAAGTAT AAAGGAGCTT ATTATTCATC AGAAATACAA |
| 1455 | AATGTCAGAA GGCAGTTACG ATATTGCCTT AATAAAGCTT CAGACACCGT TGAATTATAC |
| 1515 | TGAATTCCAA AAACCAATAT GCCTGCCTTC CAAAGCTGAC ACAAATACAA TTTATACCAA |
| 1575 | CTGCTGGGTG ACTGGATGGG GCTACACAAA GGAACGAGGT GAGACCCAAA ATATTGTACA |
| 1635 | AAAGGCAACT ATTCCCTTGG TACCAAATGA AGAATGCCAG AAAAAATATA GAGATTATGT |
| 1695 | TATAACCAAG CAGATGATCT GTGCTGGCTA CAAAGAAGGT GCAATAGATG CTTGTAACGG |
| 1755 | AGATTCCGGT GGCCCCTTAG TTTGCAAACA TAGTGGAAGG TCGCAGTTGC TCGGTATCAC |
| 1815 | CAGCTGGGGT GAAGGCTCTG CCCGCAAGGA GCAACCAGGA GTCTACACCA AACTTGCTGA |
| 1875 | GTACATTGAC TCGATATTGG AGAACATACA GAGCAGCAAG GAAAGAGCTC TGGAGACATC |
| 1935 | TCCAGCATGA CCAGGCTGGC TACTGACGGG CAACAGCCCA GCTGGCACCA GCTTTACCAC |
| 1995 | CTGCCCTCAA GTCCTACTAG AGCTCCAGAG TTCTCTTCTG CAAAATGTCG ATAGTGGTGT |
| 2055 | CTACCTCGCA TCCTTACCAT AGGATTAAAA GTCCAAATGT AGACACAGTT GCTAAAGACA |
| 2115 | GCGCCATGCT CAAGCGTGCT TCCT |

(fragment from 1516 to 1658 in bold. band size: 143)
(gene length is 2444, only region from 1035 to 2138 shown)

TABLE 2
SPECIES #2. Mouse Plasma Kallikrein Gene Fragment used for competitive
PCR

| | |
|---|---|
| 2326 | GTAAGGGAGA TTCCGGTGGC CCCTTAGTCT GTAAACACAG TGGACGGTGG CAGTTGGTGG |
| 2386 | GTATCACCAG CTGGGGTGAA CGCTGCGGCC GCAAGGACCA ACCAGGAGTC TACACCAAAC |
| 2446 | TTTCTGAGTA CATGGACTGG ATATTGGAGA AGACACAGAG CAGTGATGTA AGAGCTCTGG |
| 2506 | AGACATCTTC AGCCTGAGGA GGCTGGGTAC CAACGAGGAA GAACCCAGCT GGCTTTACCA |
| 2566 | CCTGCCCTCA AGGCAAACTA GAGCTCCAGG ATTCTCCGCT GTAAAATGTT GATAATGGTG |
| 2626 | TCTACCTCAC ATCCGTATCA TTGGATTGAA AATTCAAGTG TACATATACT TGCTGAAGAC |
| 2686 | AGCGTTTTGC TCAAGTGTGT TTCCTGCCTT GAGTCACAGG AGCTCCAATG GGAGCATTAC |
| 2746 | AAAGATCACC AAGCTTGTTA GGAAAGAGAA TGATCAAAGG GTTTTATTAG GTAATGAAAT |
| 2806 | GTCTAGATGT GATGCAATTG AAAAAAAGAC CCCAGATTTT AGCACAGTCC TTGGGACCAT |
| 2866 | TTTCATGTAA CTGTTGACTT TGGACCTCAG CAGATCTCAG AGTTACCTGT CCACTTCTGA |
| 2926 | CATTTGTTTA TTAGAGCCTG ATGCTATTCT TTCAAGTGGA GCAAAAAAAA AAAAAAAAAA |
| 2986 | AAAAA |

(fragment from 2807 to 2902 in bold. band size: 96)
(gene length is 2990, only region from 2326 to 2990 shown)

TABLE 3
Human Plasma Kallikrein Gene and Protein Sequence.
>CG56155-01 2245 nt

AGAACAGCTTGAAGACCGTTCATTTTTAAGTCACAACAGACTCACCTCCAAGAAGCAATT
GTGTTTTCAGAATGATTTTATTCAAGCAAGCAACTTATTTCATTTCCTTGTTTGCTACAG
TTTCCTGTGGATGTCTGACTCAACTCTATGAAAACGCCTTCTTCAGAGGTGCGGATGTAG
CTTCCATGTACACCCCAAATGCCCAATACTGCCAGATGAGGTGCACATTCCACCCAAGGT
GTTTGCTATTCAGTTTTCTTCCAGCAAGTTCAATCAATGACATGGACAAAAGGTTTGGTT
GCTTCTTGAAAGATAGTGTTACAGGAACCCTGCCAAAAGTACATCGAACAGGTGCAGTTT
CTGGACATTCCTTGAAGCAATGTGGTCATCAAATAAGTGCTTGCCATCGAGACATTTATA
AAGGAGTTGATATGAGAGGAHTCAATTTTAATCTGTCTAAGGTTAGCAGTGTTGAAGAAT
CCCAAAAAAGGTGCACCAATAACATTCGCTGCCAGTTTTTTTCATATGCCACGCAAACAT
TTCACAAGGCAGAGTACCGGAACAATTCCCTATTAAAGTACAGTCCCGGAGGAACACCTA
CCGCTATAAACGTGCTGAGTAACGTGGAATCTGGATTCTCACTGAAGCCCTGTGCCCTTT
CAGAAATTGGTTGCCACATGAACATCTTCCACCATCTTCCGTTCTCAGATGTGGATGTTG
CCAGCGTTCTCACTCCAGATGCTTTTGTGTGTCGGACCATCTGCACCTATCACCCCAACT
GCCTCTTCTTTACATTCTATACAAATGTATGGAAAATCGAGTCACAAAGAAATGTTTGTC
TTCTTAAAACATCTGAAAGTGGCACACCAAGTTCCTCTACTCCTCAAGAAAACACCATAT
CTCGATATAGCCTTTTAACCTGCAAAAGAACTTTACCTGAACCCTGCCACTCTAAAATTT
ACCCTGGAGTTGACTTTGGAGGAGAACAATTGAATGTGACTTTTGTTAAACGAGTGAATC
TTTGCCAAGAGACTTGCACAAAGATGATTCGCTGTCAGTTTTTCACTTATTCTTTACTCC
CAGAAGACTGTAAGGAAGAGAAGTGTAAGTGTTTCTTAACATTATCTATGGATCGTTCTC
CAACTAGGATTGCGTATGGGACACAAGCGAGCTCTGGTTACTCTTTGACATTGTGTAACA
CTGGGGACAACTCTGTCTGCACAACAAAAACAACCACACGCATTGTTGGAGGAACAAACT
CTTCTVCCCGACAGTGGCCCTCGCAGGTGAGCCTGCAGGTGAAGCTGACAGCTCAGAGGC
ACCTGTGTGGAGGGTCACTCATAGGACACCAGTGGGTCCTCACTGCTGCCCACTGCTTTG
ATGGGCTTCCCCTGCAGGATGTTTGGCGCATCTATAGTGGCATTTTAAATCTGTCAGACA
TTACAAAAGATACACCTTTCTCACAAATAAAAGAGATTATTATTCACCAAAACTATAAAG
TCTCAGAAGGGAATCATGATATCGCCTTGATAAAACTCCAGGCTCCTTTCAATTACACTG
AATTCCAAAAACCAATATGCCTACCTTCCAAACGTGACACAAGCACAATTTATACCAACT
GTTGGGTAACCGGATGGGGCTTCTCGAACGAGAAAGGTGAAATCCAAAATATTCTACAAA
AGGTAAATATTCCTTTCGTAACAAATGAAGAATCCCACAAAAQATATCAACATTATAAAA
TAACCCAACGGATCGTCTGTGCTGCCTATAAAGAAGGGGGAAAAGATGCTTGTAAGGGAG
ATTCAGCTGGTCCCTTAGTTTGCAAACACAACGGAATGTGGCGTTTGGTGGGCATCACAA
GCTGGGGTGAAGGCTGTGCCCGCAGGGAGCAACCTGGTGTCTACACCAAAGTCGCTGAGT
ACATGGACTGGATTTTAGAGAAAACACAGAGCAGTGATGGAAAAGCTCAGATGCAGTCAC
CACCATGAGAAGCAGTCCAGAGTCTAGGCAATTTTTACAACCTGAGTTCAAGTCAAATTC
TGAGCCTGGGGGGTCCTCATCTGCAAAGCATGGAGAGTGGCATCTTCTTTGCATCCTAAG
GACGAAAGACACAGTGCACTCAGAGCTCGTGAGGACAATGTCTGCTGAAGCCCGCTTTCA
GCACCCCGTAACCAGGGGCTCACAATGCGAGGTCGCAACTGAGATCTCCATGACTGTGTG
TTGTGAAATAAAATGGTGAAAGATC

TABLE 4
Amino acid sequence for Human Plasma Kallikrein
ORF Start: 72 ORF Stop: 1986 Frame: 3
Human Plasma Kallikrein Protein Sequence:
>CG56155-01-prot 638 aa

MILFKQATYFISLFATVSCGCLTQLYENAFFRGGDVASMYTPMAQYCQMR
CTFHPRCLLFSELPASSINDMEKRFCCFLKDSVTGTLPKVHRTGAVSGHS
LKQCGHQISACURDIYKGVDMRCVNPNVSKVSSVEECQKRCTNNIRCQFF
SYATQTFHKAEYRNNCLLKYSPGGTPTAIKVLSNVESGFSLKPCALSEIG
CHMNIFQHLAFSDVDVARVLTPDAFVCRTICTYHPNCLFFTFYTNVWKIE
SQRNVCLLKTSESGTPSSSTPQENTISGYSLLTCKRTLPEPCHSKIYPGV
DFGGEELNVTFVKGVNVCQETCTKMIRCQFFTYSLLPEDCKEEKCKCFLR
LSMDGSPTRIAYGTQGSSGYSLRLCNTGDNSVCTTKTSTRIVGGTNSSWG
EWPWQVSLQVKLTAQRHLCGGSLICHQWVLTAAHCFDCLPLQDVWRIYSG
ILNLSDITKDTPFSQIKEIIIHQNYKVSEGNHDIALIKLQAPLNYTEFQK
PICLPSKGDTSTIYTNCWVTGWGFSKEKGEIQNILQKVNIPLVTNEECQK
RYQDYKITQRNVCAGYKEGCKDACKGDSGGPLVCKHNGMWRLVGITSWGE
GCARREQPGVYTKVAEYMDWILEKTQSSDGKAQMQSPA

[image]

[image]

Human Plasma Kallikrein

Locus: 4q35

Extracellular

In addition to the human version of the Plasma Kallikrein identified as being differentially expressed in the experimental study, other variants have been identified by direct sequencing of cDNAs derived from many different human tissues and from sequences in public databases. No splice-form variants have been identified at CuraGen whereas several amino acid-changing cSNPs were identified. These are found below. The preferred variant of all those identified, to be used for screening purposes, is CG56155-01.

TABLE 6
The variants of the human Plasma Kallikrein obtained
from direct cloning and/or public databases

| DNA | | | AA | AA | public |
|---|---|---|---|---|---|
| Position | Strand | Alleles | Position | Change | SNP # |
| 499 | Minus | A:G | 143 | Asn => Ser | |
| 726 | Minus | G:T | 219 | Val => Phe | |
| 726 | Minus | T:G | 219 | Val => Phe | |
| 1212 | Minus | T:G | 381 | Ser => Ala | |
| 1272 | Minus | T:G | 401 | Glu => | |
| 1832 | Minus | C:T | 587 | Asn => Asn | |
| 2073 | Minus | G:A | 0 | | |
| 2073 | Minus | A:G | 0 | | |

Expression Profiles:

Table 7. CG56155-01: Plasma kallikrein—isoform1, submitted to study DDAT on 01/09/01 by sspaderna; clone status=FIS; novelty=Public; ORF start=72, ORF stop=1986, frame=3; 2245 bp.

Expression of gene CG56155-01 was assessed using the primer-probe set Ag1688, described in Table 7. Results of the RTQ-PCR runs are shown in Tables 8 and 9.

TABLE 7
Probe Name Ag1688

| | | | Start | SEQ ID |
|---|---|---|---|---|
| Primers | Sequences | Length | Position | NO: |
| Forward | 5′-tcagaagggaatcatgatatcg-3′ | 22 | 1503 | 627 |
| Probe | TET-5′-ccttgataaaactccaggctcctttga-3′-TAMRA | 27 | 1525 | 628 |
| Reverse | 5′-tttggaaggtaggcatattgg-3′ | 21 | 1572 | 629 |

TABLE 8
Panel 1.3D

| | Rel. Exp. (%) |
|---|---|
| | Ag1688, Run |
| Tissue Name | 147249266 |
| Liver adenocarcinoma | 0.0 |
| Pancreas | 6.7 |
| Pancreatic ca. CAPAN 2 | 0.2 |
| Adrenal gland | 1.8 |
| Thyroid | 3.8 |
| Salivary gland | 1.5 |
| Pituitary gland | 6.1 |
| Brain (fetal) | 0.5 |
| Brain (whole) | 3.6 |
| Brain (amygdala) | 3.3 |
| Brain (cerebellum) | 0.4 |
| Brain (hippocampus) | 6.2 |
| Brain (substantia nigra) | 1.0 |
| Brain (thalamus) | 2.1 |
| Cerebral Cortex | 6.3 |
| Spinal cord | 3.1 |
| glio/astro U87-MG | 0.0 |
| glio/astro U-118-MG | 0.0 |
| astrocytoma SW1783 | 0.0 |
| neuro*; met SK-N-AS | 0.2 |
| astrocytoma SF-539 | 0.0 |
| astrocytoma SNB-75 | 0.1 |
| glioma SNB-19 | 0.2 |
| glioma U251 | 1.2 |
| glioma SF-295 | 0.0 |
| Heart (Fetal) | 0.2 |
| Heart | 1.6 |
| Skeletal muscle (Fetal) | 0.7 |
| Skeletal muscle | 1.2 |
| Bone marrow | 0.5 |
| Thymus | 3.2 |
| Spleen | 1.0 |
| Lymph node | 2.9 |
| Colorectal | 0.8 |
| Stomach | 3.3 |
| Small intestine | 6.2 |
| Colon ca. SW480 | 0.0 |
| Colon ca.* SW620 (SW480 met) | 0.0 |
| Colon ca. HT29 | 0.0 |
| Colon ca. HCT-116 | 0.0 |
| Colon ca. CaCo-2 | 0.2 |
| CC Well to Mod Diff (ODO3866) | 0.0 |
| Colon ca. HCC-2998 | 0.2 |
| Gastric ca. (liver met) NCI-N87 | 4.4 |
| Bladder | 3.1 |
| Trachea | 3.0 |
| Kidney | 6.8 |
| Kidney (fetal) | 9.2 |
| Renal ca. 786-0 | 0.0 |
| Renal ca. A498 | 1.7 |
| Renal ca. RXF 393 | 0.0 |
| Renal ca. ACHN | 0.0 |
| Renal ca. UO-31 | 0.0 |
| Renal ca. TK-10 | 0.0 |
| Liver | 100.0 |
| Liver (fetal) | 99.3 |
| Liver ca. (hepatoblast) HepG2 | 0.0 |
| Lung | 1.3 |
| Lung (fetal) | 1.8 |
| Lung ca. (small cell) LX-1 | 0.0 |
| Lung ca. (small cell) NCI-H69 | 0.0 |
| Lung ca. (s. cell var.) SHP-77 | 0.8 |
| Lung ca. (large cell) NCI-H460 | 0.0 |
| Lung ca. (non-sm. cell) A549 | 0.2 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW900 | 0.2 |
| Lung ca. (squam.) NCI-H596 | 0.0 |
| Mammary gland | 2.9 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.2 |
| Ovarian ca. OVCAR-4 | 0.0 |
| Ovarian ca. OVCAR-5 | 0.3 |
| Ovarian ca. OVCAR-8 | 0.0 |
| Ovarian ca. IGROV-1 | 0.0 |
| Ovarian ca. (ascites) SK-OV-3 | 1.0 |
| Uterus | 1.4 |
| Placenta | 0.4 |
| Prostate | 1.0 |
| Prostate ca.* (bone met) PC-3 | 0.0 |
| Testis | 6.1 |
| Melanoma Hs688(A).T | 0.4 |
| Melanoma* (met) Hs688(B).T | 0.9 |
| Melanoma UACC-62 | 0.0 |
| Melanoma M 14 | 0.0 |
| Melanoma LOX IMVI | 0.0 |
| Melanoma* (met) SK-MEL-5 | 0.0 |
| Adipose | 0.5 |

TABLE 9
Panel 5 Islet

| | Rel. Exp. (%) |
|---|---|
| | Ag1688, Run |
| Tissue Name | 226587524 |
| 97457_Patient-02go_adipose | 41.2 |
| 97476_Patient-07sk_skeletal muscle | 9.9 |
| 97477_Patient-07ut_uterus | 8.1 |
| 97478_Patient-07pl_placenta | 0.0 |
| 99167_Bayer Patient 1 | 84.7 |
| 97482_Patient-08ut_uterus | 2.4 |
| 97483_Patient-08pl_placenta | 0.0 |
| 97486 Patient-09sk_skeletal muscle | 8.0 |
| 97487_Patient-09ut_uterus | 9.6 |
| 97488_Patient-09pl_placenta | 0.0 |
| 97492_Patient-10ut_uterus | 0.0 |
| 97493_Patient-10pl_placenta | 0.0 |
| 97495_Patient-11go_adipose | 0.0 |
| 97496_Patient-11sk_skeletal muscle | 52.9 |
| 97497_Patient-11ut_uterus | 35.8 |
| 97498_Patient-11pl_placenta | 10.5 |
| 97500_Patient-12go_adipose | 0.0 |
| 97501_Patient-12sk_skeletal muscle | 35.4 |
| 97502_Patient-12ut_uterus | 20.7 |
| 97503_Patient-12pl_placenta | 0.0 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 11.4 |
| 94713_Donor 2 AD - B_adipose | 0.0 |
| 94714_Donor 2 AD - C_adipose | 29.1 |
| 94742_Donor 3 U - A_Mesenchymal Stem Cells | 19.2 |
| 94743_Donor 3 U - B_Mesenchymal Stem Cells | 0.0 |
| 94730_Donor 3 AM - A_adipose | 15.0 |
| 94731_Donor 3 AM - B_adipose | 37.9 |
| 94732_Donor 3 AM - C_adipose | 0.0 |
| 94733_Donor 3 AD - A_adipose | 39.2 |
| 94734_Donor 3 AD - B_adipose | 11.4 |
| 94735_Donor 3 AD - C_adipose | 34.4 |
| 77138_Liver_HepG2untreated | 8.4 |
| 73556_Heart_Cardiac stromal cells (primary) | 0.0 |
| 81735_Small Intestine | 100.0 |
| 72409_Kidney_Proximal Convoluted Tubule | 9.9 |
| 82685_Small intestine_Duodenum | 70.2 |
| 90650_Adrenal_Adrenocortical adenoma | 25.5 |
| 72410_Kidney_HRCE | 10.4 |
| 72411_Kidney_HRE | 7.2 |
| 73139_Uterus_Uterine smooth muscle cells | 0.0 |

Biochemistry and Cell Line Expression

Plasma Kallikrein is a protease which is implicated in the conversion of plasminogen to the plasmin. Plasma Kallikrein activity was measured usually by spectrophotometric assays using artificial fluorescent peptide substrates. Plasma Kallikrein is commercially available enzyme with known inhibitors. The procedure of purification of Plasma Kallikrein from serum by affinity chromatography was described in literature. Cell lines expressing the

Plasma Kallikrein can be obtained from the RTQ-PCR results shown above. These and other Plasma Kallikrein expressing cell lines could be used for screening purposes.

Rationale for Use as a Diagnostic and/or Target for Small Molecule Drugs and Antibody Therapeutics.

1. Plasminogen activation, followed by fibrinolysis, is implicated recently in adipose differentiation by remodeling of the fibronectin-rich ECM of the preadipocytes. Knock out of the plasminogen gene in mouse lead to the reduction of fat deposit.

2. Plasma Kallikrein activates plasminogen, thus promoting adipose differentiation.

3. Plasma Kallikrein is significantly down-regulated in the liver of mice with the lean phenotype, which may cause disruption of the adipose differentiation ion this strain.

4. Taken in total, the data indicates that an inhibitor/antagonist of the human Plasma Kallikrein would be beneficial in the treatment of obesity.

SPECIES #1 A gene fragment of the rat Plasma Kallikrein was initially found to be down-regulated by 2 fold in MB.01 study in the liver of SHR rat relative to normal control rat strain using CuraGen's GeneCallinG™ method of differential gene expression. Additionally, the expression of the enzyme was increased in the response to troglitazone treatment. A differentially expressed rat gene fragment migrating, at approximately 142.3 nucleotides in length (FIG. 1a.—vertical line) was definitively identified as a component of the rat Plasma Kallikrein cDNA (in the graphs, the abscissa is measured in lengths of nucleotides and the ordinate is measured as signal response). The method of competitive PCR was used for conformation of the gene assessment. The electropherogram peaks corresponding to the gene fragment of the rat Plasma Kallikrein are ablated when a gene-specific primer (see below) competes with primers in the linker-adaptors during the PCR amplification. The peaks at 142.3 nt in length are ablated in the sample from both the SHR and control rats.

SPECIES #2 The gene fragments corresponding to the mouse Plasma Kallikrein were found to be down-regulated by 52.1 fold in liver tissues of normal mice relative to the lean mice. A differentially expressed mouse gene fragment migrating, at approximately 96 nucleotides in length (FIG. 1a.—red vertical line) was definitively identified as a component of the mouse Plasma Kallikrein cDNA by the method of competitive PCR. The electropherogramatic peaks corresponding to the gene fragment of the mouse Plasma Kallikrein are ablated when a gene-specific primer (see below) competes with primers in the linker-adaptors during the PCR amplification. The peaks at 96 nt in length are ablated in the sample from both the normal and lean mice.

The sequence of the nucleotide-long gene fragment and the gene-specific primers used for competitive PCR are indicated on the cDNA sequence of the Plasma Kallikrein and shown below in bold. The gene-specific primers at the 5′ and 3′ ends of the fragment are in color.

[image]

[image]

### Example F

CG56155-03 Expression Data:

Construction of the mammalian expression vector pCEP4/Sec. The oligonucleotide primers, pSec-V5-His Forward (CTCGTCCTCGAGGGTAAGCCTATCCCT AAC) and the pSec-V5-His Reverse (CTCGTCGGGCCCCTGATCAGCGGGTTTAAAC), were designed to amplify a fragment from the pcDNA3.1-V5His (Invitrogen, Carlsbad, Calif.) expression vector. The PCR product was digested with XhoI and ApaI and ligated into the XhoI/ApaI digested pSecTag2 B vector (Invitrogen, Carlsbad Calif.). The correct structure of the resulting vector, pSecV5His, was verified by DNA sequence analysis. The vector pSecV5His was digested with PmeI and NheI, and the PmeI-NheI fragment was ligated into the BamHI/Klenow and NheI treated vector pCEP4 (Invitrogen, Carlsbad, Calif.). The resulting vector was named as pCEP4/Sec.

Expression of CG56155-03 in human embryonic kidney 293 cells. A 0.4 kb BamHI-XhoI fragment containing the CG56155-03 sequence was subcloned into BamHI-XhoI digested pCEP4/Sec to generate plasmid 1061. The resulting plasmid 1061 was transfected into 293 cells using the LipofectaminePlus reagent following the manufacturer's instructions (Gibco/BRL). The cell pellet and supernatant were harvested 72 h post transfection and examined for CG56155-03 expression by Western blot (reducing conditions) using an anti-V5 antibody. FIG. 1 shows that CG56155-03 is expressed as a 74 kDa protein secreted by 293 cells.

[image]

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequenced selected from the group consisting of SEQ ID NO:2n, wherein n is an integer between 1 and 226.

2. An isolated polypeptide comprising an amino acid sequence selected from the group consisting of SEQ ID NO:2n, wherein n is an integer between 1 and 226.

3. An isolated polypeptide comprising an amino acid sequence which is at least 95% identical to an amino acid sequence selected from the group consisting of SEQ ID NO:2n, wherein n is an integer between 1 and 226.

4. An isolated polypeptide, wherein the polypeptide comprises an amino acid sequence comprising one or more conservative substitutions in the amino acid sequence selected from the group consisting of SEQ ID NO:2n, wherein n is an integer between 1 and 226.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide,
thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 226 or a biologically active fragment thereof.

20. An isolated nucleic acid molecule comprising a nucleic acid sequence selected from the group consisting of SEQ ID NO:2n-1, wherein n is an integer between 1 and 226.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n-1, wherein n is an integer between 1 and 226.

23. An isolated nucleic acid molecule encoding the mature form of a polypeptide having an amino acid sequence selected from the group consisting of SEQ ID NO:2n, wherein n is an integer between 1 and 226.

24. An isolated nucleic acid molecule comprising a nucleic acid selected from the group consisting of 2n-1, wherein n is an integer between 1 and 226.

25. The nucleic acid molecule of claim 20, wherein said nucleic acid molecule hybridizes under stringent conditions to the nucleotide sequence selected from the group consisting of SEQ ID NO: 2n-1, wherein n is an integer between 1 and 226, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n-1, wherein n is an integer between 1 and 226.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n-1, wherein n is an integer between 1 and 226.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.
